(19) United States
(12) Patent Application Publication
(10) Pub. No.: US 2025/0248302 A1
(43) Pub. Date: Jul. 31, 2025

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Hiroaki TOYOSHIMA, Tokyo (JP); Yusuke TAKAHASHI, Tokyo (JP); Shota TANAKA, Tokyo (JP); Keitaro YAMADA, Tokyo (JP)

(73) Assignee: IDEMITSU KOSAN CO.,LTD., Tokyo (JP)

(21) Appl. No.: 18/707,232

(22) PCT Filed: Nov. 4, 2022
(86) PCT No.: PCT/JP2022/041112

§ 371 Date: May 3, 2024

(30) Foreign Application Priority Data
Nov. 5, 2021 (JP) 2021-181511
Nov. 5, 2021 (JP) 2021-181513
Jan. 28, 2022 (JP) 2022-012100
Jan. 28, 2022 (JP) 2022-012156

(51) Int. Cl. H10K 85/60; H10K 50/15; H10K 50/858; H10K 102/00

(52) U.S. Cl. CPC H10K 85/633; H10K 85/60; H10K 85/636; H10K 50/156; H10K 50/858; H10K 85/615; H10K 85/626; H10K 85/6572; H10K 85/6574; H10K 2102/351

(57) ABSTRACT

An organic EL device includes a first hole transporting zone including a first anode-side organic layer and a second anode-side organic layer, in which at least one of the first anode-side organic layer or the second anode-side organic layer contains an additive material, and when the first anode-side organic layer contains a fourth material as the additive material, Numerical Formula N1 and N2 are satisfied:

$${NM}_{1} > {NM}_{2} \qquad ( Numerical Formula N1 )$$

$${NM}_{14} > N {M}_{10} \qquad ( Numerical Formula N2 )$$

where NM₁ represents a refractive index of a constituent material in the first anode-side organic layer; NM₂ represents a refractive index of a constituent material in the second anode-side organic layer; NM₁₀ represents a refractive index of the constituent material in the first anode-side organic layer not containing the fourth material; and NM₁₄ represents a refractive index of the constituent material in the first anode-side organic layer containing the fourth material.

### TECHNICAL FIELD

The present invention relates to an organic electroluminescence device and an electronic device.

### BACKGROUND ART

An organic electroluminescence device (hereinafter, occasionally referred to as “organic EL device”.) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

The performance of the organic EL device is evaluable in terms of, for instance, luminance, emission wavelength, chromaticity, luminous efficiency, drive voltage, and lifetime.

In Patent Literatures 1 to 7, for instance, studies have been made to improve performance of an organic EL device. Patent Literatures 1 to 7 disclose an organic EL device including a hole transporting zone including a plurality of layers.

### CITATION LIST

### Patent Literature(s)

Patent Literature 1 International Publication No. WO 2021/065773
Patent Literature 2 JP 2021-061305 A
Patent Literature 3 International Publication No. WO 2011/110262
Patent Literature 4 International Publication No. WO 2014/044344
Patent Literature 5 International Publication No. WO 2020/225071
Patent Literature 6 International Publication No. WO 2016/064110
Patent Literature 7 Korean Publication Patent No. 2016-0054855

### SUMMARY OF THE INVENTION

### Problem(s) to be Solved by the Invention

An object of the invention is to provide an organic electroluminescence device with improved device performance, and to provide an electronic device including the organic electroluminescence device.

### Means for Solving the Problem(s)

According to an aspect of the invention, there is provided an organic electroluminescence device, including: a cathode; an anode; a first emitting region disposed between the cathode and the anode; and a first hole transporting zone disposed between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed between the anode and the first emitting region in this order from a side to the anode, the first anode-side organic layer does not contain a compound comprised in the second anode-side organic layer, the first anode-side organic layer contain a first organic material and a second organic material, a content of the second organic material in the first anode-side organic layer is less than 50 mass %, the second anode-side organic layer contains a third organic material, the third organic material is an amine compound having at least one substituted or unsubstituted amino group in a molecule, at least one of the first anode-side organic or the second anode-side organic layer contains an additive material, the additive material contained in the first anode-side organic layer is a fourth material, the additive material contained in the second anode-side organic layer is a fifth material, the fourth material and the fifth material are each independently an organic material or a metal atom-containing material, the first organic material, the second organic material, the third organic material, the fourth material, and the fifth material are mutually different, and the first anode-side organic layer and the second anode-side organic layer satisfy a numerical formula (Numerical Formula N1) and at least one of numerical formulae (Numerical Formula N2 and Numerical Formula N3) below.

$${NM}_{1} > {NM}_{2} \qquad ( Numerical Formula N1 )$$

$${NM}_{14} > N {M}_{10} \qquad ( Numerical Formula N2 )$$

$${NM}_{20} > N {M}_{25} \qquad ( Numerical Formula N3 )$$

In the formulae (Numerical Formula N1), (Numerical Formula N2), and (Numerical Formula N3):
NM₁ represents a refractive index of a constituent material contained in the first anode-side organic layer;
NM₂ represents a refractive index of a constituent material contained in the second anode-side organic layer;
NM₁₀ represents a refractive index of the constituent material contained in the first anode-side organic layer not containing the fourth material;
NM₁₄ represents a refractive index of the constituent material contained in the first anode-side organic layer containing the fourth material;
NM₂₀ represents a refractive index of the constituent material contained in the second anode-side organic layer not containing the fifth material; and
NM₂₅ represents a refractive index of the constituent material contained in the second anode-side organic layer containing the fifth material.

According to another aspect of the invention, there is provided an organic electroluminescence device, including: a cathode; an anode; a first emitting region disposed between the cathode and the anode; and a first hole transporting zone disposed between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed between the anode and the first emitting region in this order from a side to the anode, the first anode-side organic layer includes a first organic material and a second organic material, a content of the second organic material in the first anode-side organic layer is less than 50 mass %, the first anode-side organic layer includes an additive material, the additive material contained in the first anode-side organic layer is a fourth material, the first organic material, the second organic material, and the fourth material are mutually different, and a difference n₄−n₁ between a refractive index of the fourth material n₄ and a refractive index of the first organic material n₁ is 0.05 or more.

According to still another aspect of the invention, there is provided an organic electroluminescence device, including: a cathode; an anode; a first emitting region disposed between the cathode and the anode; and a first hole transporting zone disposed between the anode and the first emitting region, in which the first emitting region includes at least one emitting layer, the first hole transporting zone includes at least a first anode-side organic layer, a second anode-side organic layer, and a third anode-side organic layer, the first anode-side organic layer, the second anode-side organic layer, and the third anode-side organic layer are disposed between the anode and the first emitting region in this order from a side close to the anode, the second anode-side organic layer contains a second organic material, the second organic material is an amine compound having at least one substituted or unsubstituted amino group in a molecule, the third anode-side organic layer has a film thickness of 20 nm or more, the third anode-side organic layer contains a sixth organic material, the third organic material is an amine compound having at least one substituted or unsubstituted amino group in a molecule, at least one of the second anode-side organic or the third anode-side organic layer contains an additive material, the additive material contained in the second anode-side organic layer is a fourth material, the additive material contained in the third anode-side organic layer is a fifth material, the fourth material and the fifth material are each independently an organic material or a metal atom-containing material, the second organic material, the third organic material, the fourth material, and the fifth material are mutually different, and the second anode-side organic layer and the third anode-side organic layer satisfy a numerical formula (Numerical Formula NX1) and at least one of numerical formulae (Numerical Formula NX2 and Numerical Formula NX3) below.

$${NM}_{2} > {NM}_{3} \qquad ( Numerical Formula N X1 )$$

$${NM}_{24} > N {M}_{20} \qquad ( Numerical Formula N X2 )$$

$${NM}_{30} > N {M}_{35} \qquad ( Numerical Formula N X3 )$$

In the formulae (Numerical Formula NX1), (Numerical Formula NX2), and (Numerical Formula NX3):
NM₂ represents a refractive index of a constituent material contained in the second anode-side organic layer;
NM₃ represents a refractive index of a constituent material contained in the third anode-side organic layer;
NM₂₀ represents a refractive index of the constituent material contained in the second anode-side organic layer not containing the fourth material;
NM₂₄ represents a refractive index of the constituent material contained in the second anode-side organic layer containing the fourth material;
NM₃₀ represents a refractive index of the constituent material contained in the third anode-side organic layer not containing the fifth material; and
NM₃₅ represents a refractive index of the constituent material contained in the third anode-side organic layer containing the fifth material.

According to a further aspect of the invention, there is provided an organic electroluminescence device, including: a cathode; an anode; a first emitting region disposed between the cathode and the anode; and a first hole transporting zone disposed between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed between the anode and the first emitting region in this order from a side to the anode, the second anode-side organic layer contains a second organic material, the second organic material is an amine compound having at least one substituted or unsubstituted amino group in a molecule, the second anode-side organic layer contains an additive material, the additive material contained in the second anode-side organic layer is a fourth material, and a difference n₄−n₂ between a refractive index of the fourth material n₄ and a refractive index of the second organic material n₂ is 0.05 or more.

According to a still further aspect of the invention, there is provided an electronic device including the organic electroluminescence device according to the above aspect of the invention.

According to the aspects of the invention, there can be provided an organic electroluminescence device with improved performance and an electronic device including the organic electroluminescence device.

### BRIEF EXPLANATION OF DRAWINGS

FIG. 1 schematically depicts an exemplary arrangement of organic electroluminescence devices according to a first exemplary embodiment, a second exemplary embodiment, and a third exemplary embodiment.

FIG. 2 schematically depicts another exemplary arrangement of the organic electroluminescence devices according to the first exemplary embodiment, the second exemplary embodiment, and the third exemplary embodiment.

FIG. 3 schematically depicts still another exemplary arrangement of the organic electroluminescence devices according to the first exemplary embodiment, the second exemplary embodiment, and the third exemplary embodiment.

FIG. 4 schematically depicts a further exemplary arrangement of the organic electroluminescence devices according to the first exemplary embodiment, the second exemplary embodiment, and the third exemplary embodiment.

FIG. 5 schematically depicts a still further exemplary arrangement of the organic electroluminescence devices according to the first exemplary embodiment, the second exemplary embodiment, and the third exemplary embodiment.

FIG. 6 schematically depicts a still further exemplary arrangement of the organic electroluminescence devices according to the first exemplary embodiment, the second exemplary embodiment, and the third exemplary embodiment.

FIG. 7 schematically depicts an exemplary arrangement of an organic electroluminescence devices according to a fourth exemplary embodiment, a fifth exemplary embodiment, and a sixth exemplary embodiment.

FIG. 8 schematically depicts another exemplary arrangement of the organic electroluminescence devices according to the fourth exemplary embodiment, the fifth exemplary embodiment, and the sixth exemplary embodiment.

FIG. 9 schematically depicts still another exemplary arrangement of the organic electroluminescence devices according to the fourth exemplary embodiment, the fifth exemplary embodiment, and the sixth exemplary embodiment.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.” A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl 10 group, benzofluoranthenyl group, perylenyl group, and monovalent aryl group derived by removing one hydrogen atom from cyclic structures represented by formulae (TEMP-1) to (TEMP-15) below.

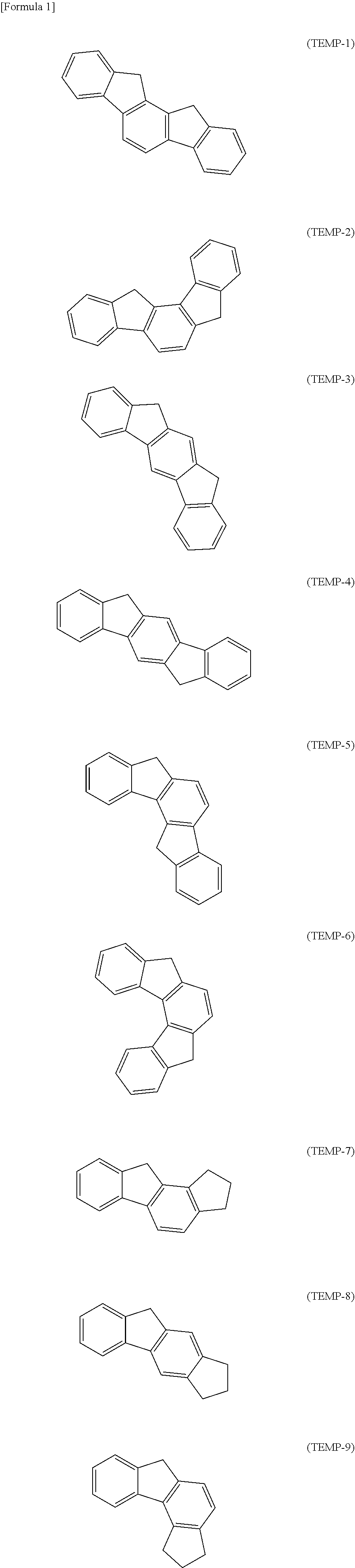

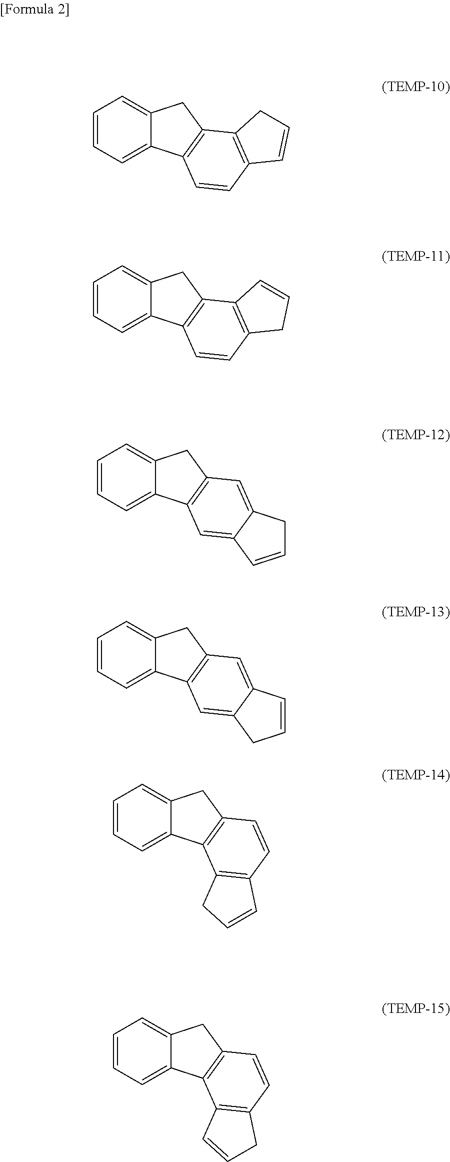

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl) fluorenyl group, 9,9-bis(4-isopropylphenyl) fluorenyl group, 9,9-bis(4-t-butylphenyl) fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

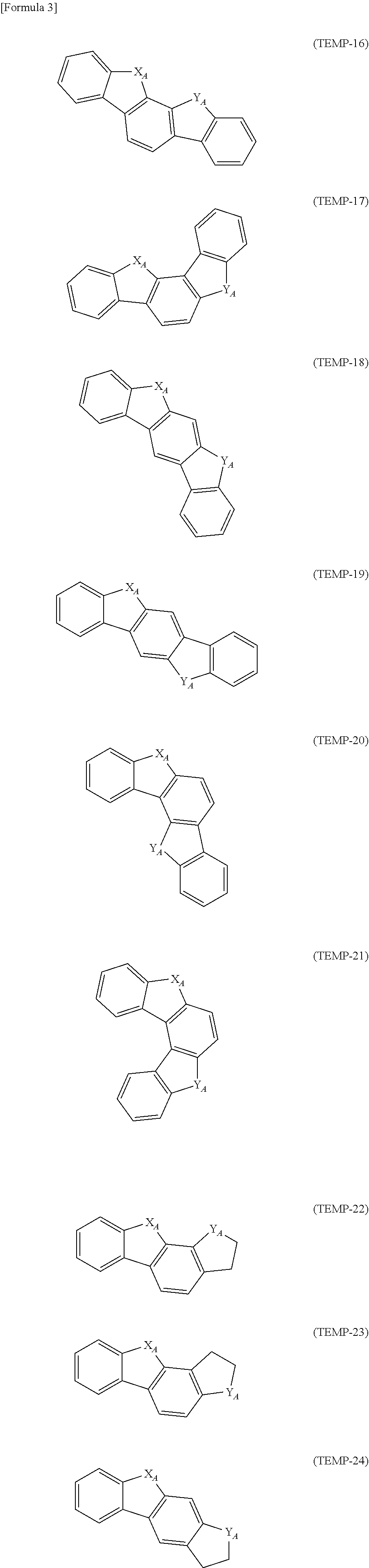

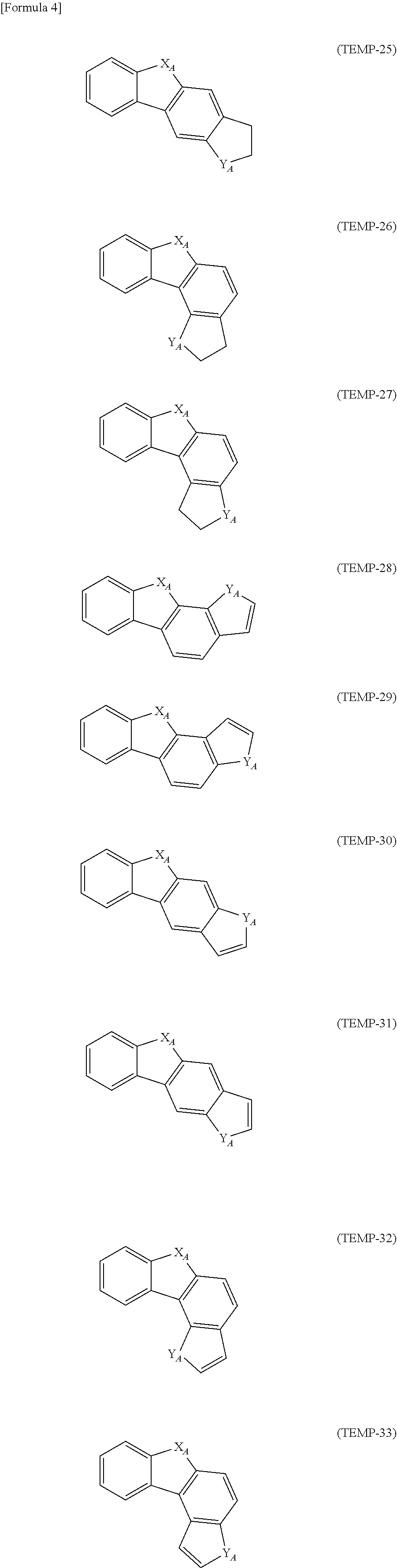

In the formulae (TEMP-16) to (TEMP-33), X_{A} and Y_{A} are each independently an oxygen atom, a sulfur atom, NH or CH₂. At least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

a (9-phenyl) carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

a phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, t-methyldibenzothiophenyl group, butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.
   Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6), where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1) (G1) (G1) are mutually the same or different;
a plurality of G2 in —Si(G1) (G2) (G2) are mutually the same or different;
a plurality of G1 in —Si(G1) (G1) (G2) are mutually the same or different;
a plurality of G2 in —Si(G2) (G2) (G2) are mutually the same or different;
a plurality of G3 in —Si(G3)(G3)(G3) are mutually the same or different; and
a plurality of G6 in —Si(G6) (G6) (G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —O(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6) (G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is occasionally referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl) carbazolyl group ((9-phenyl) carbazole-1-yl group, (9-phenyl) carbazole-2-yl group, (9-phenyl) carbazole-3-yl group, or (9-phenyl) carbazole-4-yl group), (9-biphenylyl) carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

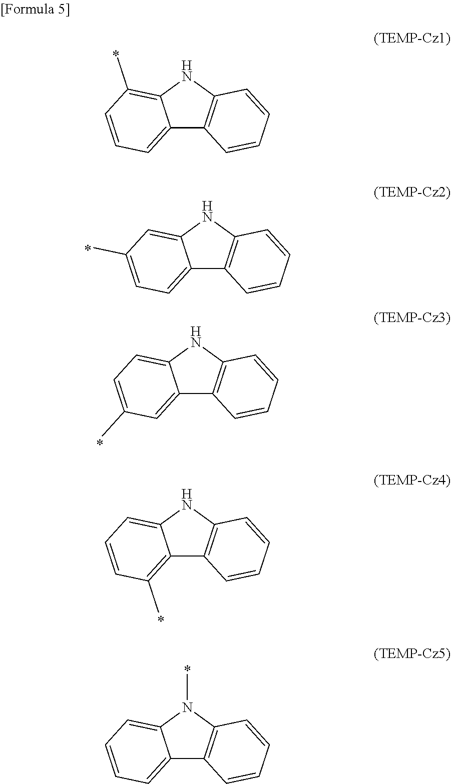

The (9-phenyl) carbazolyl group mentioned herein is, unless otherwise specified herein, specifically a group represented by one of formulae below.

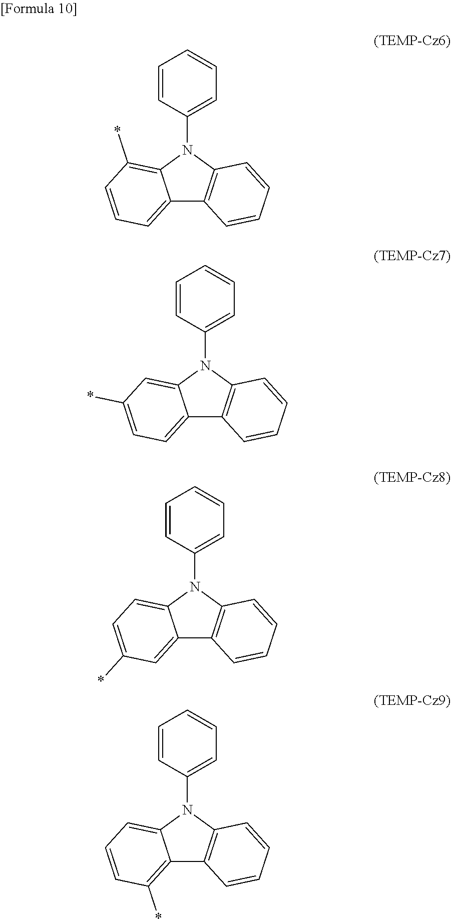

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

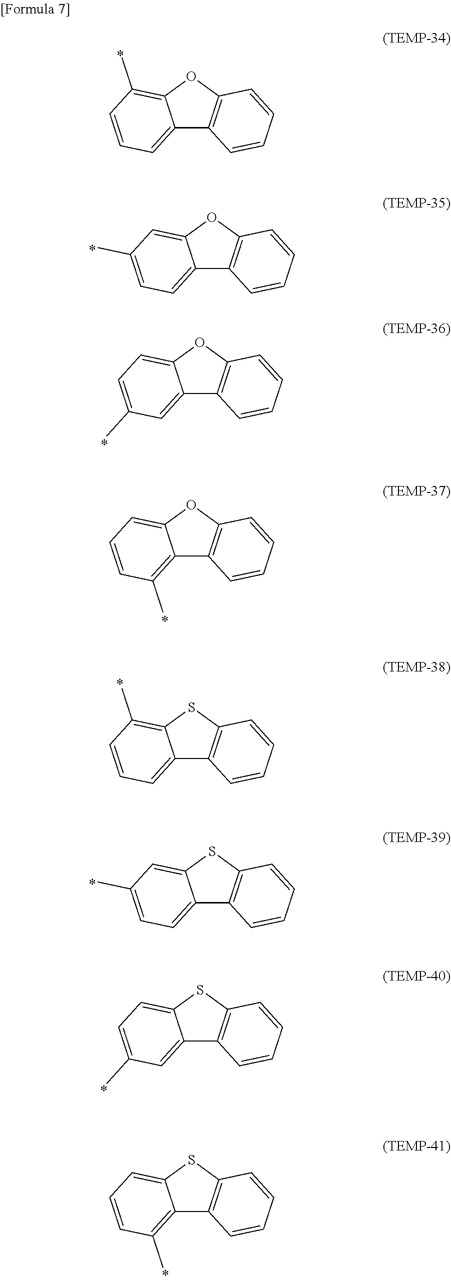

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocycle of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

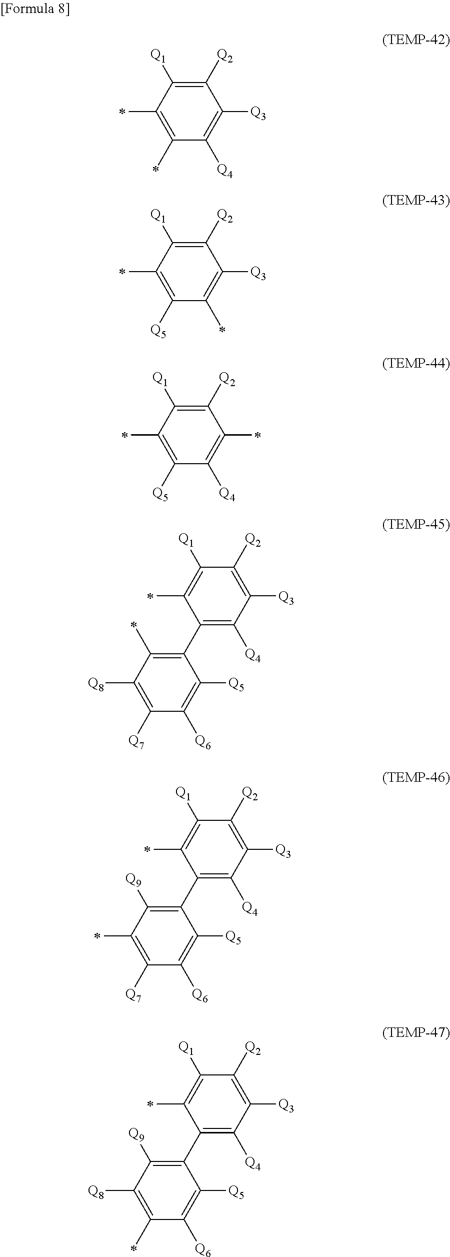

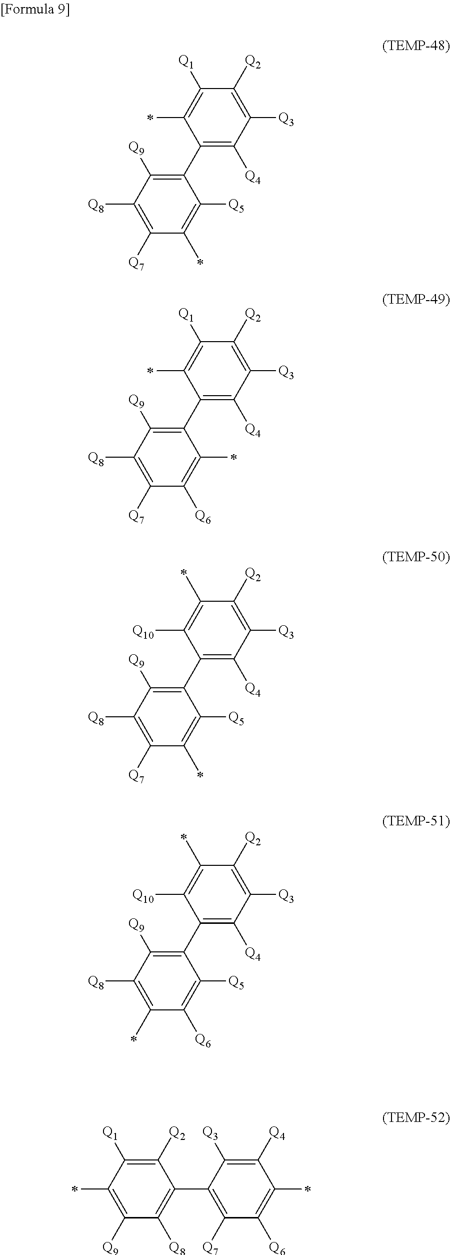

In the formulae (TEMP-42) to (TEMP-52), Q₁ to Q₁₀ are each independently a hydrogen atom or a substituent.

In the formulae (TEMP-42) to (TEMP-52), * represents a bonding position.

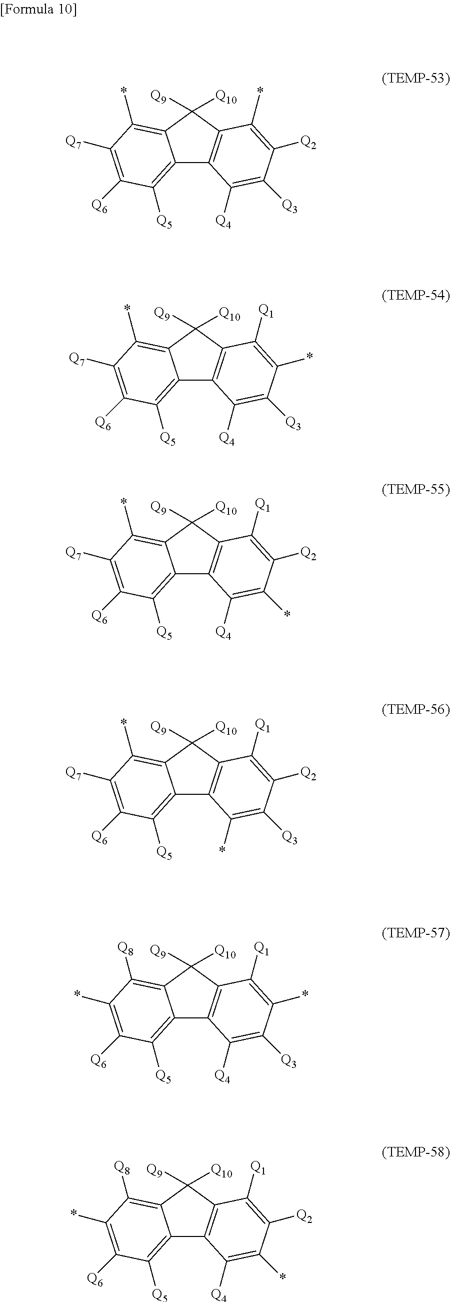

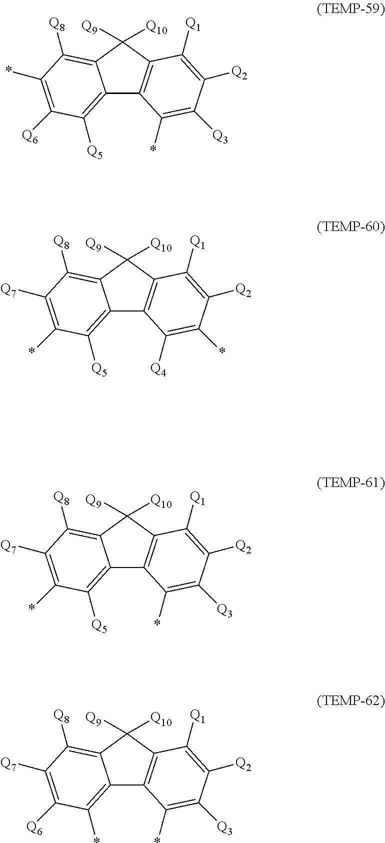

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

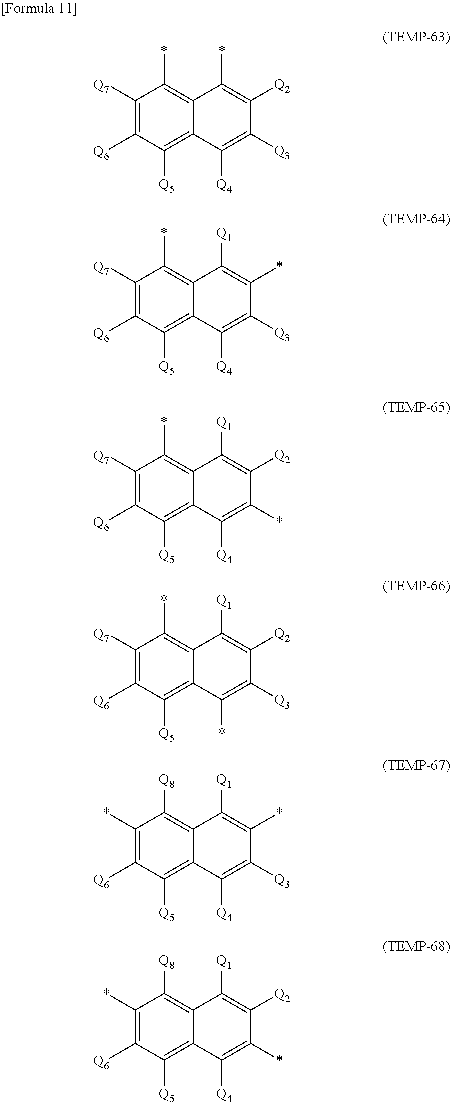

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

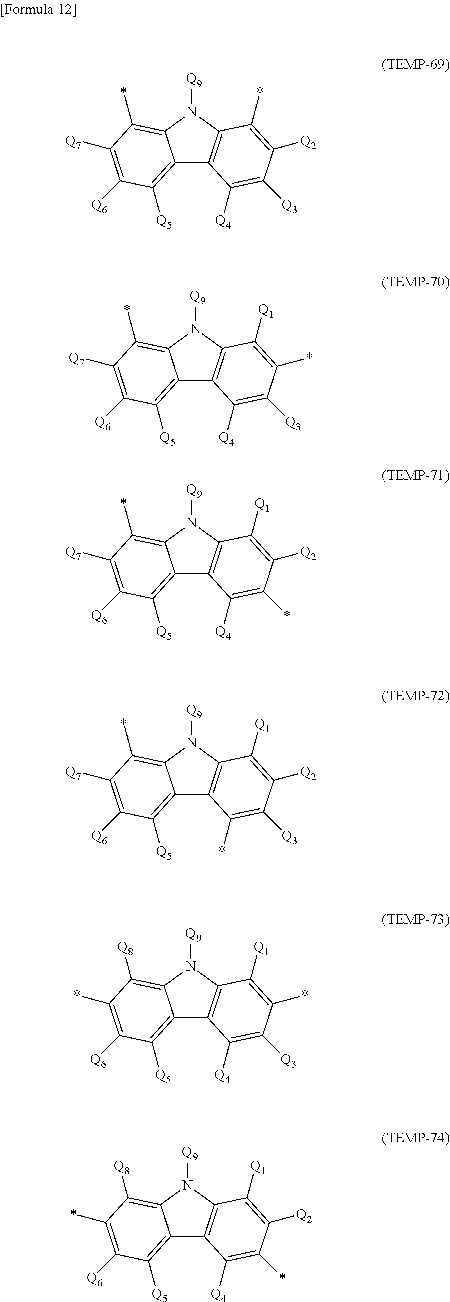

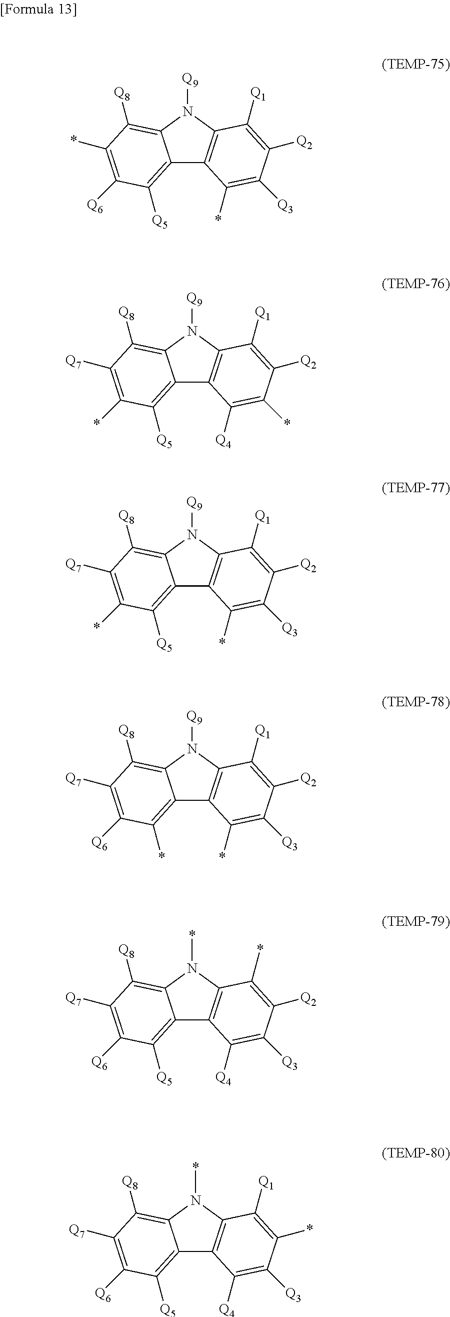

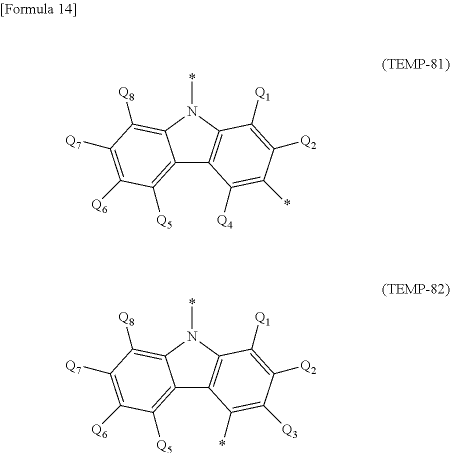

In the formulae (TEMP-69) to (TEMP-82), Q₁ to Q₉ are each independently a hydrogen atom or a substituent.

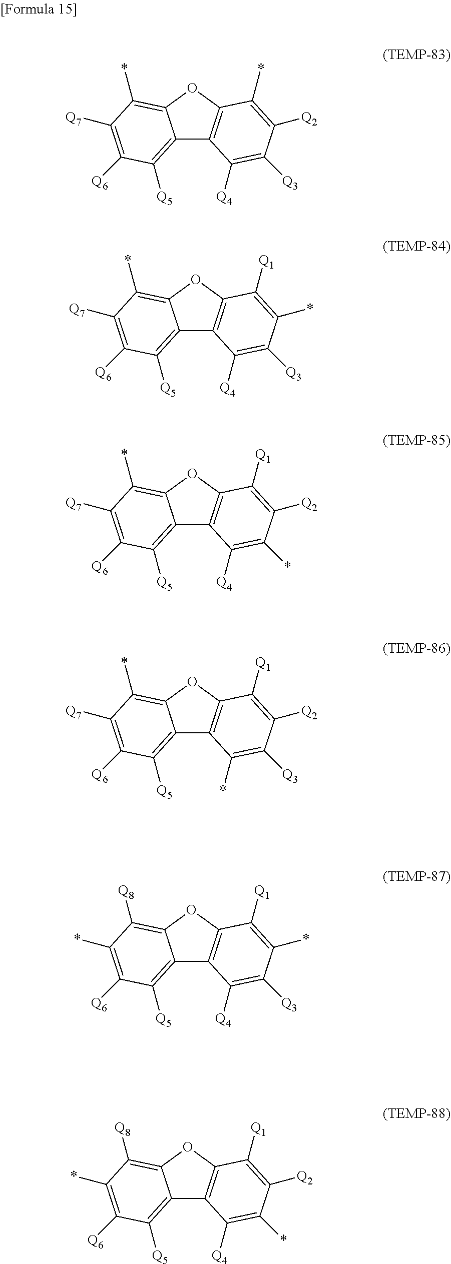

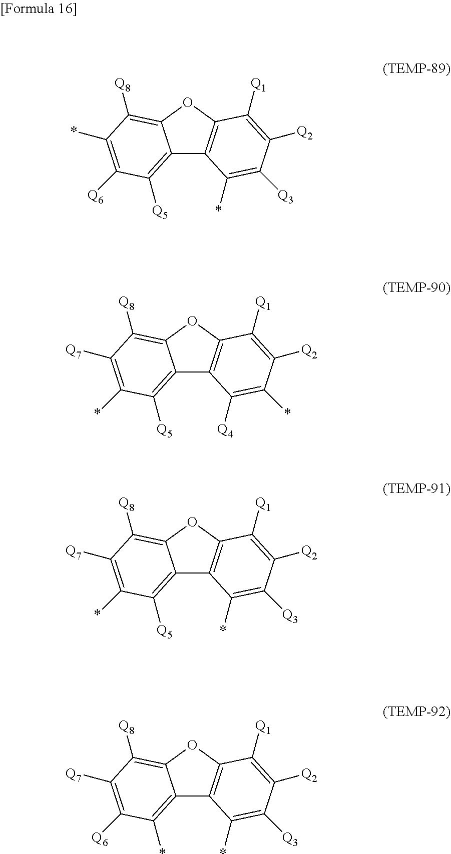

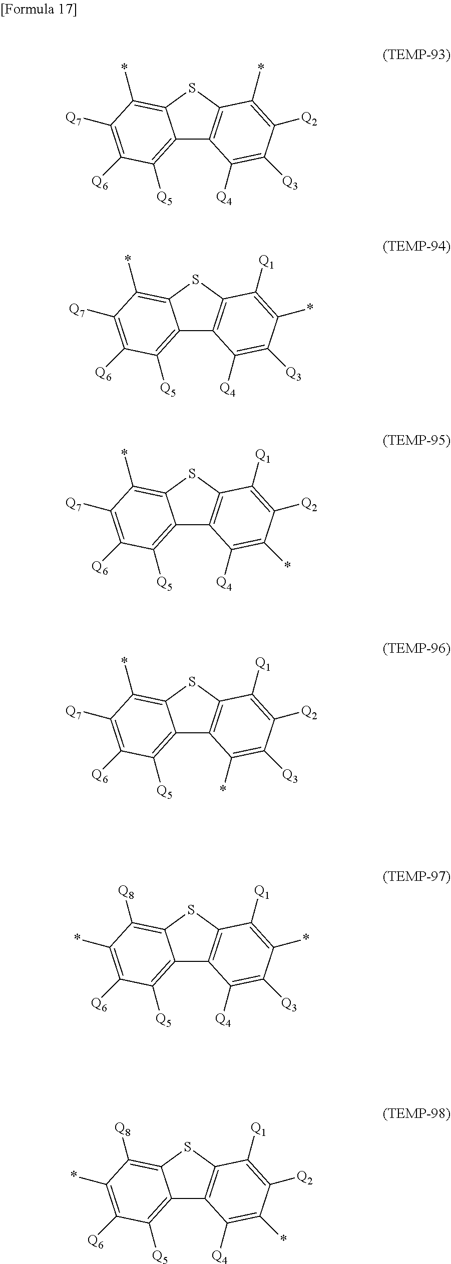

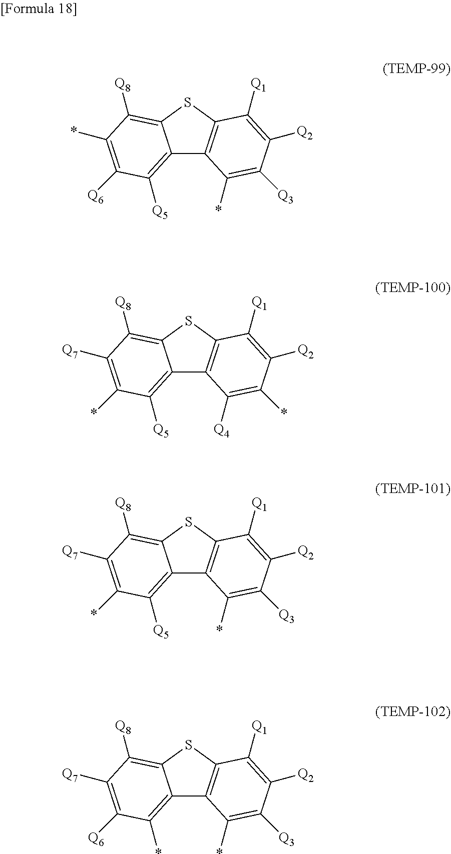

In the formulae (TEMP-83) to (TEMP-102), Q₁ to Qs are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

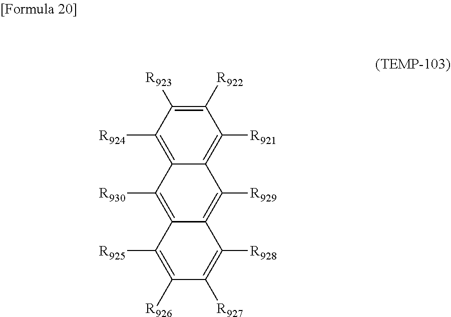

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring QB, the anthracene compound represented by the formula (TEMP-103) is represented by a formula (TEMP-104) below.

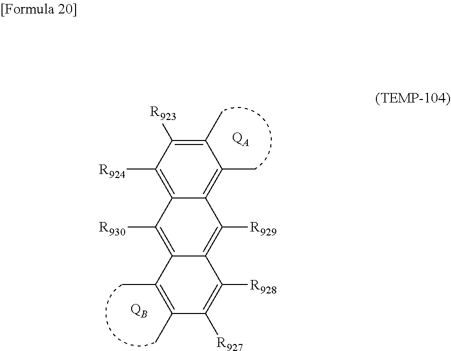

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

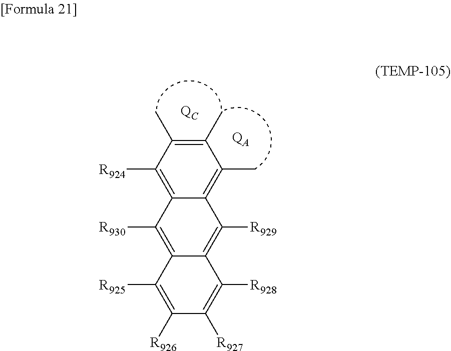

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring QB formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes any other optional element than the carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (hereinafter sometimes referred to as an “optional substituent”), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more Roos are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, and an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A≥B” means that the value A is equal to the value B, or the value A is larger than the value B.

Herein, a numerical formula represented by “A≤B” means that the value A is equal to the value B, or the value A is smaller than the value B.

### First Exemplary Embodiment

An organic electroluminescence device according to a first exemplary embodiment includes: a cathode; an anode; a first emitting region disposed between the cathode and the anode; and a first hole transporting zone disposed between the anode and the first emitting region, in which the first emitting region includes at least one emitting layer, the hole transporting zone includes at least a first anode-side organic layer and a second anode-side organic layer, the first anode-side organic layer and the second anode-side organic layer are disposed between the anode and the first emitting region in this order from a side to the anode, the first anode-side organic layer does not contain a compound contained in the second anode-side organic layer, the first anode-side organic layer contains a first organic material and a second organic material, a content of the second organic material in the first anode-side organic layer is less than 50 mass %, the second anode-side organic layer contains a third organic material, the third organic material is an amine compound having at least one substituted or unsubstituted amino group in a molecule, at least one of the first anode-side organic or the second anode-side organic layer contains an additive material, the additive material contained in the first anode-side organic layer is a fourth material, the additive material contained in the second anode-side organic layer is a fifth material, the fourth material and the fifth material are each independently an organic material or a metal atom-containing material, the first organic material, the second organic material, the third organic material, the fourth material, and the fifth material are mutually different, and the first anode-side organic layer and the second anode-side organic layer satisfy a numerical formula (Numerical Formula N1) and at least one of numerical formulae (Numerical Formula N2 and Numerical Formula N3) below.

$${NM}_{1} > {NM}_{2} \qquad ( Numerical Formula N1 )$$

$${NM}_{14} > N {M}_{10} \qquad ( Numerical Formula N2 )$$

$${NM}_{20} > N {M}_{25} \qquad ( Numerical Formula N3 )$$

In the formulae (Numerical Formula N1), (Numerical Formula N2), and (Numerical Formula N3):
NM₁ represents a refractive index of a constituent material contained in the first anode-side organic layer;
NM₂ represents a refractive index of a constituent material contained in the second anode-side organic layer;
NM₁₀ represents a refractive index of the constituent material contained in the first anode-side organic layer not containing the fourth material;
NM₁₄ represents a refractive index of the constituent material contained in the first anode-side organic layer containing the fourth material;
NM₂₀ represents a refractive index of the constituent material contained in the second anode-side organic layer not containing the fifth material; and
NM₂₅ represents a refractive index of the constituent material contained in the second anode-side organic layer containing the fifth material.

According to the first exemplary embodiment, the device performance of the organic EL device is improvable. In the organic EL device of the first exemplary embodiment, the refractive index NM₁ of the constituent material contained in the first anode-side organic layer is larger than the refractive index NM₂ of the constituent material contained in the second anode-side organic layer. Light extraction efficiency of the organic EL device is improved by the refractive index NM₁ being larger than the refractive index NM₂. Further, in the organic EL device according to the first exemplary embodiment, at least one of the first anode-side organic layer or the second anode-side organic layer contains an additive material satisfying at least one of the formulae (Numerical Formula N2 and Numerical Formula N3). Therefore, according to the first exemplary embodiment, a difference NM₁−NM₂ in refractive index between the first anode-side organic layer and the second anode-side organic layer is increasable to further improve the light extraction efficiency of the organic EL device. The organic EL device of the first exemplary embodiment improves luminous efficiency as the device performance, as compared with an organic EL device having a hole transporting zone containing no additive material. An exemplary arrangement of the organic EL device of the first exemplary embodiment prolongs a lifetime as compared with an organic EL device having a hole transporting zone containing no additive material.

When the first anode-side organic layer contains a single type of compound, the refractive index NM₁ of the constituent material contained in the first anode-side organic layer corresponds to a refractive index of the single type of compound. When the first anode-side organic layer contains a plurality of types of compounds, the refractive index NM₁ of the constituent materials contained in the first anode-side organic layer corresponds to a refractive index of a mixture containing the plurality types of compounds. The refractive index NM₂, the refractive index NM₃, and refractive indices of constituent materials of other layers are defined in the same manner as the refractive index NM₁. The refractive index can be measured by a measurement method described in Examples below. Herein, a value of the refractive index at 2.7 eV in the substrate parallel direction (Ordinary direction) measured by multi-incidence angle spectroscopic ellipsometry measurement is defined as a refractive index of the measurement target material. The refractive index at 2.7 eV corresponds to a refractive index at 460 nm.

In a “common arrangement (1) among the first exemplary embodiment, the second exemplary embodiment, and the third exemplary embodiment” and a “common arrangement (3) among all the exemplary embodiments” which are later described, the organic EL device of the first exemplary embodiment will be further described.

### Second Exemplary Embodiment

An organic electroluminescence device of a second exemplary embodiment includes: a cathode; an anode; a first emitting region disposed between the cathode and the anode; and a first hole transporting zone disposed between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed in this order from a side close to the anode between the anode and the first emitting region, the first anode-side organic layer contains a first organic material and a second organic material, a content of the second organic material in the first anode-side organic layer is less than 50 mass %, the first anode-side organic layer contains an additive material, the additive material contained in the first anode-side organic layer is a fourth material, the first organic material, the second organic material, and the fourth material are mutually different, and a difference n₄−n₁ between a refractive index of the fourth material n₄ and a refractive index of the first organic material n₁ is 0.05 or more.

In the organic EL device of the second exemplary embodiment, since the refractive index difference n₄−n₁ is 0.05 or more, the fourth material as the additive material has a higher refractive index than the first organic material. By containing the fourth material in the first anode-side organic layer, the refractive index difference between the first anode-side organic layer and the second anode-side organic layer is increasable to further improve the light extraction efficiency of the organic EL device. Similar to the organic EL device of the first exemplary embodiment, the organic EL device of the second exemplary embodiment improves the luminous efficiency as the device performance as compared with an organic EL device having a hole transporting zone containing no additive material. An exemplary arrangement of the organic EL device of the second exemplary embodiment prolongs a lifetime as compared with an organic EL device having a hole transporting zone containing no additive material.

In an exemplary arrangement of the organic EL device of the second exemplary embodiment, the first anode-side organic layer does not contain the compound contained in the second anode-side organic layer.

In an exemplary arrangement of the organic EL device of the second exemplary embodiment, the second anode-side organic layer contains a third organic material.

In an exemplary arrangement of the organic EL device of the second exemplary embodiment, the third organic material is an amine compound having at least one substituted or unsubstituted amine group in a molecule.

In an exemplary arrangement of the organic EL device of the second exemplary embodiment, the second anode-side organic layer contains an additive material. The additive material contained in the second anode-side organic layer is a fifth material.

In an exemplary arrangement of the organic EL device of the second exemplary embodiment, the fifth material is an organic material or a metal atom-containing material.

In an exemplary arrangement of the organic EL device of the second exemplary embodiment, the first organic material, the second organic material, the third organic material, the fourth material, and the fifth material are mutually different.

In an exemplary arrangement of the organic EL device of the second exemplary embodiment, the first anode-side organic layer and the second anode-side organic layer satisfy the numerical formula (Numerical Formula N1) of the first exemplary embodiment and at least one of the formulae (Numerical Formula N2 and Numerical Formula N3).

In the “common arrangement (1) among the first exemplary embodiment, the second exemplary embodiment, and the third exemplary embodiment” and the “common arrangement (3) among all the exemplary embodiments” which are later described, the organic EL device of the second exemplary embodiment will be further described.

### Third Exemplary Embodiment

An organic electroluminescence device according to a third exemplary embodiment includes a cathode, an anode, a first emitting region provided between the cathode and the anode, and a first hole transporting zone provided between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed in this order from a side close to the anode between the anode and the first emitting region, the second anode-side organic layer contains a third organic material and an additive material, the additive material contained in the second anode-side organic layer is a fifth material, the third organic material and the fifth material are mutually different, and a difference n₃−n₅ between a refractive index of the third organic material n₃ and a refractive index of the fifth material n₅ is 0.02 or more.

In the organic EL device of the third exemplary embodiment, since the difference n₃−n₅ is 0.02 or more, the fifth material as the additive material has a lower refractive index than the third organic material. By containing the fifth material in the second anode-side organic layer, the refractive index difference between the first anode-side organic layer and the second anode-side organic layer is increasable to further improve the light extraction efficiency of the organic EL device. Similar to the organic EL devices of the first exemplary embodiment and the second exemplary embodiment, the organic EL device of the third exemplary embodiment improves the luminous efficiency as the device performance as compared with an organic EL device having a hole transporting zone containing no additive material. An exemplary arrangement of the organic EL device of the third exemplary embodiment prolongs a lifetime as compared with an organic EL device having a hole transporting zone containing no additive material.

In an exemplary arrangement of the organic EL device according to the third exemplary embodiment, the difference n₃−n₅ between the refractive index of the third organic material n₃ and the refractive index of the fifth material n₅ is 0.03 or more.

In an exemplary arrangement of the organic EL device of the third exemplary embodiment, the first anode-side organic layer does not contain the compound contained in the second anode-side organic layer.

In an exemplary arrangement of the organic EL device of the third exemplary embodiment, the third organic material is an amine compound having at least one substituted or unsubstituted amine group in a molecule.

In an exemplary arrangement of the organic EL device of the third exemplary embodiment, the fifth material is an organic material or a metal atom-containing material.

In an exemplary arrangement of the organic EL device of the third exemplary embodiment, the first anode-side organic layer contains a first organic material and a second organic material.

In an exemplary arrangement of the organic EL device of the third exemplary embodiment, a content of the second organic material in the first anode-side organic layer is less than 50 mass %.

In an exemplary arrangement of the organic EL device of the third exemplary embodiment, the first anode-side organic layer contains an additive material. The additive material contained in the first anode-side organic layer is a fourth material.

In an exemplary arrangement of the organic EL device of the third exemplary embodiment, the fourth material is an organic material or a metal atom-containing material.

In an exemplary arrangement of the organic EL device of the third exemplary embodiment, the first organic material, the second organic material, the third organic material, the fourth material, and the fifth material are mutually different.

In an exemplary arrangement of the organic EL device according to the third exemplary embodiment, the first anode-side organic layer and the second anode-side organic layer satisfy the numerical formula (Numerical Formula N1) of the first exemplary embodiment and at least one of the formulae (Numerical Formula N2 and Numerical Formula N3).

In the “common arrangement (1) among the first exemplary embodiment, the second exemplary embodiment, and the third exemplary embodiment” and the “common arrangement (3) among all the exemplary embodiments” which are later described, the organic EL device of the third exemplary embodiment will be further described.

### Common Arrangement (1) Among First Exemplary Embodiment, Second Exemplary Embodiment, and Third Exemplary Embodiment

Regarding the common arrangement (1) among the first exemplary embodiment, the second exemplary embodiment, and the third exemplary embodiment, an arrangement applicable to each of the first exemplary embodiment, the second exemplary embodiment, and the third exemplary embodiment described herein will be described below.

### Hole Transporting Zone

Herein, a region disposed between the anode and the first emitting region and including a plurality of organic layers is referred to as a first hole transporting zone.

The first hole transporting zone of the organic EL device of the first, second, and third exemplary embodiments includes at least the first anode-side organic layer and the second anode-side organic layer. The first anode-side organic layer and the second anode-side organic layer are disposed between the anode and the first emitting region in this order from a side close the anode.

In an exemplary arrangement of the organic EL devices of the first, second, and third exemplary embodiments, the anode is in direct contact with the first anode-side organic layer.

In an exemplary arrangement of the organic EL devices of the first, second, and third exemplary embodiments, the first anode-side organic layer is in direct contact with the second anode-side organic layer.

In an exemplary arrangement of the organic EL devices of the first, second, and third exemplary embodiments, the second anode-side organic layer is in direct contact with the first emitting region.

In an exemplary arrangement of the organic EL devices of the first, second, and third exemplary embodiments, a difference NM₁−NM₂ between a refractive index NM₁ of a constituent material contained in the first anode-side organic layer and a refractive index NM₂ of a constituent material contained in the second anode-side organic layer satisfies a relationship of a numerical formula (Numerical Formula N4) below.

$${NM}_{1} - {NM}_{2} \geq 0 \text{.05} \qquad ( Numerical Formula N4 )$$

In an exemplary arrangement of the organic EL devices of the first, second, and third exemplary embodiments, the refractive index difference NM₁−NM₂ satisfies a relationship of a numerical formula (Numerical Formula N41, Numerical Formula N42, or Numerical Formula N43) below.

$${NM}_{1} - {NM}_{2} \geq 0.1 \qquad ( Numerical Formula N41 )$$

$${NM}_{1} - {NM}_{2} \geq 0.15 \qquad ( Numerical Formula N42 )$$

$${NM}_{1} - {NM}_{2} \geq 0 \text{.20} \qquad ( Numerical Formula N43 )$$

The first anode-side organic layer contains the first organic material and the second organic material. In an exemplary arrangement of the organic EL devices of the first, second, and third exemplary embodiments, the first organic material and the second organic material are mutually different compounds. A content of the second organic material in the first anode-side organic layer is less than 50 mass %. In an exemplary arrangement of the organic EL devices of the first, second, and third exemplary embodiments, the first anode-side organic layer further contains the fourth material as the additive material.

In an exemplary arrangement of the organic EL devices of the first, second, and third exemplary embodiments, the difference n₄−n₁ between a refractive index of the fourth material n₄ and the refractive index of the first organic material n₁ is 0.05 or more.

In an exemplary arrangement of the organic EL devices of the first, second, and third exemplary embodiments, the difference n₄−n₁ between the refractive index of the fourth material n₄ and the refractive index of the first organic material n₁ is 0.07 or more.

In an exemplary arrangement of the organic EL devices of the first, second, and third exemplary embodiments, the refractive index of the fourth material n₄ is 2.0 or more. By adding the fourth material having a higher refractive index in the first anode-side organic layer, the refractive index difference between the first anode-side organic layer and the second anode-side organic layer is more increased to further improve the light extraction efficiency of the organic EL device.

In an exemplary arrangement of the organic EL devices of the first, second, and third exemplary embodiments, the first anode-side organic layer does not contain the compound contained in the second anode-side organic layer. An arrangement satisfying the above condition is, for instance, as follows: when a compound CA, a compound CB, a compound AA and a compound AB are different compounds, the second anode-side organic layer contains a single type of compound (the compound AA) and the first anode-side organic layer contains two types of compounds (the compound CA and the compound CB). Further, the above condition is also satisfied when the second anode-side organic layer contains two types of compounds (the compound AA and the compound AB) and the first anode-side organic layer contains two types of compounds (the compound CA and the compound CB). On the other hand, the above condition is not satisfied, for instance, when the second anode-side organic layer contains a single type of compound (the compound AA) and the first anode-side organic layer contains two types of compounds (the compound CA and the compound AA), because the first anode-side organic layer and the second anode-side organic layer contain the identical compound (the compound AA).

In an exemplary arrangement of the organic EL devices of the first, second, and third exemplary embodiments, the first anode-side organic layer contains the first organic material, the second organic material, and the fourth material.

In an exemplary arrangement of the organic EL devices of the first, second, and third exemplary embodiments, the refractive index NM₁₄ is larger than the refractive index NM₁₀ as shown in the numerical formula (Numerical Formula N2). By containing the fourth material as the additive material in the first anode-side organic layer, the refractive index of the first anode-side organic layer is increased. As a result, the difference NM₁−NM₂ in refractive index between the first anode-side organic layer and the second anode-side organic layer is easily increased.

In an exemplary arrangement of the organic EL devices of the first, second, and third exemplary embodiments, a content of the fourth material in the first anode-side organic layer is 50 mass % or less.

In an exemplary arrangement of the organic EL devices of the first, second, and third exemplary embodiments, the content of the fourth material in the first anode-side organic layer is 40 mass % or less.

In an exemplary arrangement of the organic EL devices of the first, second, and third exemplary embodiments, the content of the fourth material in the first anode-side organic layer is 30 mass % or less.

The hole transportability can be ensured by setting the content of the fourth material in the first anode-side organic layer at 50 mass % or less and increasing the content of the first organic material.

In an exemplary arrangement of the organic EL devices of the first, second, and third exemplary embodiments, the content of the fourth material in the first anode-side organic layer is 1 mass % or more, 5 mass % or more, or 10 mass % or more.

In an exemplary arrangement of the organic EL devices of the first, second, and third exemplary embodiments, the second anode-side organic layer contains the third organic material and the fifth material.

In the organic EL devices of the first, second, and third exemplary embodiments, the refractive index NM₂₅ is smaller than the refractive index NM₂₀ as shown in the numerical formula (Numerical Formula N3). By containing the fifth material as the additive material in the second anode-side organic layer, the refractive index of the second anode-side organic layer is decreased. As a result, the difference NM₁−NM₂ in refractive index between the first anode-side organic layer and the second anode-side organic layer is easily increased.

In an exemplary arrangement of the organic EL devices of the first, second, and third exemplary embodiments, a content of the fifth material in the second anode-side organic layer is 50 mass % or less.

In an exemplary arrangement of the organic EL devices of the first, second, and third exemplary embodiments, the content of the fifth material in the second anode-side organic layer is 40 mass % or less.

In an exemplary arrangement of the organic EL devices of the first, second, and third exemplary embodiments, the content of the fifth material in the second anode-side organic layer is 30 mass % or less.

In an exemplary arrangement of the organic EL devices of the first, second, and third exemplary embodiments, the content of the fifth material in the second anode-side organic layer is 1 mass % or more, 5 mass % or more, or 10 mass % or more.

In an exemplary arrangement of the organic EL devices of the first, second, and third exemplary embodiments, the first anode-side organic layer contains the first organic material, the second organic material, and the fourth material and the second anode-side organic layer contains the third organic material and the fifth material. In this arrangement, since the first anode-side organic layer and the second anode-side organic layer satisfy both of the numerical formulae (Numerical Formula N2 and Numerical Formula N3), the difference NM₁−NM₂ in refractive index between the first anode-side organic layer and the second anode-side organic layer is more easily increased.

In an exemplary arrangement of the organic EL devices of the first, second, and third exemplary embodiments, a total film thickness of the first hole transporting zone is in a range from 25 nm to 150 nm.

In an exemplary arrangement of the organic EL devices of the first, second, and third exemplary embodiments, the total film thickness of the first hole transporting zone is in a range from 25 nm to 85 nm.

In an exemplary arrangement of the organic EL devices of the first, second, and third exemplary embodiments, the total film thickness of the first hole transporting zone is in a range from 90 nm to 130 nm.

In an exemplary arrangement of the organic EL devices of the first, second, and third exemplary embodiments, a film thickness of the first anode-side organic layer is 20 nm or less.

In an exemplary arrangement of the organic EL devices of the first, second, and third exemplary embodiments, the film thickness of the first anode-side organic layer is 3 nm or more.

In an exemplary arrangement of the organic EL devices of the first, second, and third exemplary embodiments, a film thickness of the second anode-side organic layer is 20 nm or more. The light extraction efficiency is easily improved when the film thickness of the second anode-side organic layer satisfying the relationship of the numerical formula (Numerical Formula N1) is 20 nm or more.

In an exemplary arrangement of the organic EL devices of the first, second, and third exemplary embodiments, the film thickness of the second anode-side organic layer is in a range from 20 nm to 70 nm.

In an exemplary arrangement of the organic EL devices of the first, second, and third exemplary embodiments, the first hole transporting zone includes the third anode-side organic layer between the first anode-side organic layer and the second anode-side organic layer. In this arrangement, the first anode-side organic layer, the third anode-side organic layer, and the second anode-side organic layer are disposed in this order from a side close to the anode.

In an exemplary arrangement of the organic EL devices of the first, second, and third exemplary embodiments, the first anode-side organic layer is in direct contact with third anode-side organic layer.

In an exemplary arrangement of the organic EL devices of the first, second, and third exemplary embodiments, the second anode-side organic layer is in direct contact with third anode-side organic layer.

In an exemplary arrangement of the organic EL devices of the first, second, and third exemplary embodiments, the third anode-side organic layer contains a sixth organic material.

In an exemplary arrangement of the organic EL devices of the first, second, and third exemplary embodiments, the sixth organic material, the first organic material, the second organic material, the third organic material, the fourth material, and the fifth material are mutually different.

In an exemplary arrangement of the organic EL devices of the first, second, and third exemplary embodiments, a film thickness of the third anode-side organic layer is 20 nm or more.

In an exemplary arrangement of the organic EL devices of the first, second, and third exemplary embodiments, the second anode-side organic layer and the third anode-side organic layer satisfy a numerical formula (Numerical Formula N5) below.

$$N {M}_{3} > N {M}_{2} \qquad ( Numerical Formula N5 )$$

In the numerical formula (Numerical Formula N5):
NM₂ represents a refractive index of a constituent material contained in the second anode-side organic layer; and
NM₃ represents a refractive index of a constituent material contained in the third anode-side organic layer.

In an exemplary arrangement of the organic EL devices of the first, second, and third exemplary embodiments, the first hole transporting zone includes a fourth anode-side organic layer between the second anode-side organic layer and the first emitting region. In an exemplary arrangement of the organic EL devices of the first, second, and third exemplary embodiments, the fourth anode-side organic layer contains a seventh organic material.

In an exemplary arrangement of the organic EL devices of the first, second, and third exemplary embodiments, the fourth anode-side organic layer is in direct contact with the second anode-side organic layer.

In an exemplary arrangement of the organic EL devices of the first, second, and third exemplary embodiments, the fourth anode-side organic layer is in direct contact with the first emitting region.

In an exemplary arrangement of the organic EL devices of the first, second, and third exemplary embodiments, the fourth anode-side organic layer is an electron blocking layer.

Preferably, the electron blocking layer permits transport of holes and blocks electrons from reaching a layer provided closer to the anode (e.g., the hole transporting layer) beyond the electron blocking layer.

In an exemplary arrangement of the organic EL devices of the first, second, and third exemplary embodiments, the fourth anode-side organic layer may block excitons generated in the emitting layer from transferring to the layers provided closer to the anode (e.g., the first anode-side organic layer, the second anode-side organic layer, and the third anode-side organic layer) beyond the fourth anode-side organic layer so as to avoid excited energy from leaking out from the emitting layer to neighboring layers.

In an exemplary arrangement of the organic EL devices of the first, second, and third exemplary embodiments, a film thickness of the fourth anode-side organic layer is 15 nm or less.

In an exemplary arrangement of the organic EL devices of the first, second, and third exemplary embodiments, the film thickness of the fourth anode-side organic layer is 10 nm or less.

In an exemplary arrangement of the organic EL devices of the first, second, and third exemplary embodiments, the film thickness of the first anode-side organic layer is 3 nm or more.

In an exemplary arrangement of the organic EL devices of the first, second, and third exemplary embodiments, the film thickness of the fourth anode-side organic layer is smaller than the film thickness of the second anode-side organic layer. The organic EL devices of the first, second, and third exemplary embodiments are considered to prolong the lifetime by including the fourth anode-side organic layer (preferably the electron blocking layer) having the smaller film thickness than the second anode-side organic layer.

In an exemplary arrangement of the organic EL devices of the first, second, and third exemplary embodiments, the first hole transporting zone includes a first mixture layer between the first anode-side organic layer and the second anode-side organic layer. In this arrangement, the first anode-side organic layer, the first mixture layer, and the second anode-side organic layer are disposed in this order from a side close to the anode.

In an exemplary arrangement of the organic EL devices of the first, second, and third exemplary embodiments, the first mixture layer contains the first organic material, the second organic material, the third organic material, and at least one of the fourth material or the fifth material as the additive material.

In an exemplary arrangement of the organic EL devices of the first, second, and third exemplary embodiments, a film thickness of the first mixture layer may be 10 nm or more. In an exemplary arrangement of the organic EL devices of the first, second, and third exemplary embodiments, the film thickness of the first mixture layer is 50 nm or less. In an exemplary arrangement of the organic EL devices of the first, second, and third exemplary embodiments, the film thickness of the first mixture layer is smaller than the film thickness of the first anode-side organic layer and smaller than the film thickness of the second anode-side organic layer.

In an exemplary arrangement of the organic EL devices of the first, second, and third exemplary embodiments, the first mixture layer may be formed between the first anode-side organic layer and the second anode-side organic layer when the first anode-side organic layer and the second anode-side organic layer are successively formed in the same deposition chamber.

In an exemplary arrangement of the organic EL devices of the first, second, and third exemplary embodiments, in a case where the first hole transporting zone includes the third anode-side organic layer between the first anode-side organic layer and the second anode-side organic layer, the first hole transporting zone includes a second mixture layer between the first anode-side organic layer and the third anode-side organic layer. In this arrangement, the first anode-side organic layer, the second mixture layer, the third anode-side organic layer, and the second anode-side organic layer are disposed in this order from a side close to the anode.

In an exemplary arrangement of the organic EL devices of the first, second, and third exemplary embodiments, the second mixture layer contains the first organic material, the second organic material, and the six organic material. In a case where the first anode-side organic layer contains the fourth material as the additive material, the second mixture layer also contains the fourth material.

In an exemplary arrangement of the organic EL devices of the first, second, and third exemplary embodiments, in a case where the first hole transporting zone includes the third anode-side organic layer between the first anode-side organic layer and the second anode-side organic layer, the first hole transporting zone includes a third mixture layer between the third anode-side organic layer and the second anode-side organic layer. In this arrangement, the first anode-side organic layer, the third anode-side organic layer, the third mixture layer, and the second anode-side organic layer are disposed in this order from a side close to the anode.

In an exemplary arrangement of the organic EL devices of the first, second, and third exemplary embodiments, the third mixture layer contains the third organic material and the six organic material. In a case where the second anode-side organic layer contains the fifth material as the additive material, the third mixture layer also contains the fifth material.

In an exemplary arrangement of the organic EL devices of the first, second, and third exemplary embodiments, the first anode-side organic layer, the second mixture layer, the third anode-side organic layer, the third mixture layer, and the second anode-side organic layer are disposed in this order from a side close to the anode.

In an exemplary arrangement of the organic EL devices of the first, second, and third exemplary embodiments, film thicknesses of the second mixture layer and the third mixture layer may be each independently 10 nm or more.

In an exemplary arrangement of the organic EL devices of the first, second, and third exemplary embodiments, the film thicknesses of the second mixture layer and the third mixture layer are each independently 50 nm or less.

In an exemplary arrangement of the organic EL devices of the first, second, and third exemplary embodiments, the film thickness of the second mixture layer is smaller than the film thickness of the first anode-side organic layer and smaller than the film thickness of the third anode-side organic layer.

In an exemplary arrangement of the organic EL devices of the first, second, and third exemplary embodiments, the film thickness of the third mixture layer is smaller than the film thickness of the second anode-side organic layer and smaller than the film thickness of the third anode-side organic layer.

In an exemplary arrangement of the organic EL devices of the first, second, and third exemplary embodiments, the second mixture layer may be formed between the first anode-side organic layer and the third anode-side organic layer when the first anode-side organic layer and the third anode-side organic layer are successively formed in the same deposition chamber.

In an exemplary arrangement of the organic EL devices of the first, second, and third exemplary embodiments, the third mixture layer may be formed between the second anode-side organic layer and the third anode-side organic layer when the second anode-side organic layer and the third anode-side organic layer are successively formed in the same deposition chamber.

### Hole Transporting Zone Material

The organic materials contained in the first hole transporting zone (i.e., the first organic material, the third organic material, the sixth organic material, and the seventh organic material) may be occasionally referred to as a hole transporting zone material.

In an exemplary arrangement of the organic EL devices of the first, second, and third exemplary embodiments, the first organic material, the third organic material, the sixth organic material, and the seventh organic material are each independently a monoamine compound having one substituted or unsubstituted amino group in a molecule, or a diamine compound having two substituted or unsubstituted amino groups in a molecule.

In an exemplary arrangement of the organic EL devices of the first, second, and third exemplary embodiments, the first organic material, the third organic material, the sixth organic material, and the seventh organic material are each independently at least one compound selected from the group consisting of a compound represented by a formula (C1) below and a compound represented by a formula (C3) below.

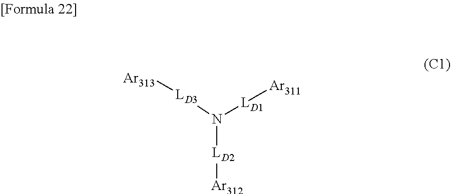

In the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C2} are present, the plurality of R_{C3} are mutually the same or different; and
L_{D1}, L_{D2}, and Los are each independently a single bond, a substituted or unsubstituted arylene group having 6 to 50 ring carbon atoms, or a substituted or unsubstituted divalent heterocyclic group having 5 to 50 ring atoms.

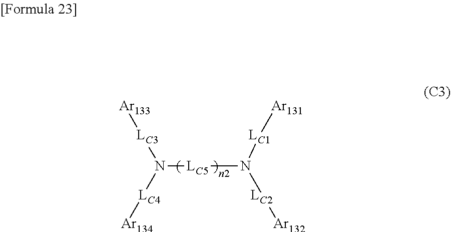

In the formula (C3):
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n₂ is 1, 2, 3, or 4;
when n₂ is 1, L_{C5} is a substituted or unsubstituted arylene group having 6 to 50 ring carbon atoms, or a substituted or unsubstituted divalent heterocyclic group having 5 to 50 ring atoms;
when n₂ is 2, 3, or 4, a plurality of L_{C5} are mutually the same or different;
when n₂ is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

In an exemplary arrangement of the organic EL devices of the first, second, and third exemplary embodiments, a substituent for the “substituted or unsubstituted” group in the compound represented by the formula (C1) and the compound represented by the formula (C3) is not a group represented by —N(R_{C6})(R_{C7}), in which R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of R_{C6} are mutually the same or different, and a plurality of R_{C7} are mutually the same or different.

In an exemplary arrangement of the organic EL devices of the first, second, and third exemplary embodiments, a first amino group represented by a formula (C3-1) below and a second amino group represented by a formula (C3-2) below are an identical group.

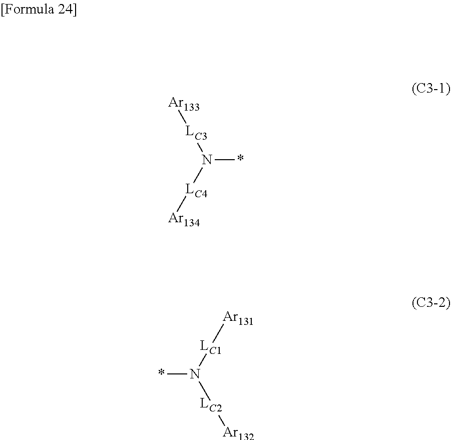

In the formulae (C3-1) and (C3-2), * each represent a bonding position to L_{C5}.

In an exemplary arrangement of the organic EL devices of the first, second, and third exemplary embodiments, the first amino group represented by the formula (C3-1) and the second amino group represented by the formula (C3-2) may be mutually different groups.

In an exemplary arrangement of the organic EL devices of the first, second, and third exemplary embodiments, the compound represented by the formula (C1) is a compound represented by a formula (C11) below.

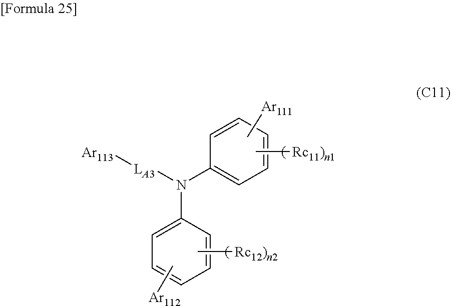

In the formula (C11):
Ar₁₁₁, Ar₁₁₂, Ar₁₁₃, and L_{A3} respectively represent the same as Ar₃₁₁, Ar₃₁₂, Ar₃₁₃, and L_{D3} in the formula (C1);
n₁ and n₂ are 4;
a plurality of R_{C11} are mutually the same or different;
at least one combination of adjacent two or more of a plurality of R_{C11} are mutually bonded to form a substituted or unsubstituted monocyclic ring, mutually bonded to form a substituted or unsubstituted fused ring, or not mutually bonded;
a plurality of R_{C12} are mutually the same or different;
at least one combination of adjacent two or more of a plurality of R_{C12} are mutually bonded to form a substituted or unsubstituted monocyclic ring, mutually bonded to form a substituted or unsubstituted fused ring, or not mutually bonded; and
R_{C11} and R_{C12} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms.

In the compound represented by the formula (C1), at least one of Ar₃₁₁, Ar₃₁₂, or Ar₃₁₃ is preferably a group selected from the group consisting of groups represented by formulae (21a), (21b), (21c), (21d), and (21e) below.

In the compound represented by the formula (C11), at least one of Ar₁₁₁, Ar₁₁₂, or Ar₁₁₃ is preferably a group selected from the group consisting of groups represented by the formulae (21a), (21b), (21c), (21d), and (21e) below.

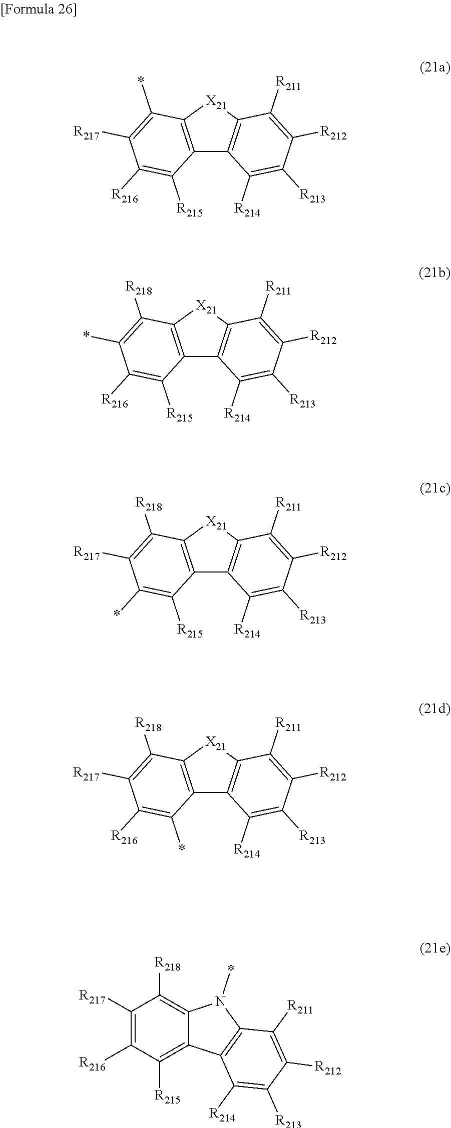

In the formulae (21a), (21b), (21c), (21d), and (21e):
X₂₁ is NR₂₁, CR₂₂R₂₃, an oxygen atom, or a sulfur atom;
when a plurality of X₂₁ are present, the plurality of X₂₁ are mutually the same or different;
when X₂₁ is CR₂₂R₂₃, a combination of R₂₂ and R₂₃ are mutually bonded to form a substituted or unsubstituted monocyclic ring, mutually bonded to form a substituted or unsubstituted fused ring, or not mutually bonded;
R₂₁, and R₂₂ and R₂₃ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded;
R₂₁₁ to R₂₁₈ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms; and
* in the formulae (21a), (21b), (21c), (21d), and (21e) are each independently a bonding position to L_{D1}, L_{D2}, L_{D3}, L_{A1}, L_{A2}, and L_{A3}.

Ar₁₁₁, Ar₁₁₂, and Ar₁₁₃ not being a group selected from the group consisting of groups represented by the formulae (21a), (21b), (21c), (21d), and (21e) are each independently preferably a substituted or unsubstituted aryl group having 6 to 30 ring carbon atoms, more preferably a substituted or unsubstituted phenyl group or a substituted or unsubstituted biphenyl group.

In the compound represented by the formula (C1), it is also preferable that two of Ar₃₁₁, Ar₃₁₂, and Ar₃₁₃ are each a group selected from the group consisting of groups represented by the formulae (21a), (21b), (21c), (21d), and (21e) and the remaining one of Ar₃₁₁, Ar₃₁₂, and Ar₃₁₃ is a substituted or unsubstituted aryl group having 6 to 30 ring carbon atoms.

In the compound represented by the formula (C11), it is also preferable that two of Ar₁₁₁, Ar₁₁₂, and Ar₁₁₃ are each a group selected from the group consisting of groups represented by the formulae (21a), (21b), (21c), (21d), and (21e) and the remaining one of Ar₁₁₁, Ar₁₁₂, and Ar₁₁₃ is a substituted or unsubstituted aryl group having 6 to 30 ring carbon atoms.

In the compound represented by the formula (C1), it is also preferable that one of Ar₃₁₁, Ar₃₁₂, and Ar₃₁₃ is a group selected from the group consisting of groups represented by the formulae (21a), (21b), (21c), (21d), and (21e) and the remaining two of Ar₃₁₁, Ar₃₁₂, and Ar₃₁₃ are each a substituted or unsubstituted aryl group having 6 to 30 ring carbon atoms.

In the compound represented by the formula (C11), it is also preferable that one of Ar₁₁₁, Ar₁₁₂, and Ar₁₁₃ is a group selected from the group consisting of groups represented by the formulae (21a), (21b), (21c), (21d), and (21e) and the remaining two of Ar₁₁₁, Ar₁₁₂, and Ar₁₁₃ are each a substituted or unsubstituted aryl group having 6 to 30 ring carbon atoms.

In an exemplary arrangement of the organic EL devices of the first, second, and third exemplary embodiments, the first anode-side organic layer contains at least one compound selected from the group consisting of a compound represented by a formula (cHT2-1), a compound represented by a formula (cHT2-2), and a compound represented by a formula (cHT2-3) below.

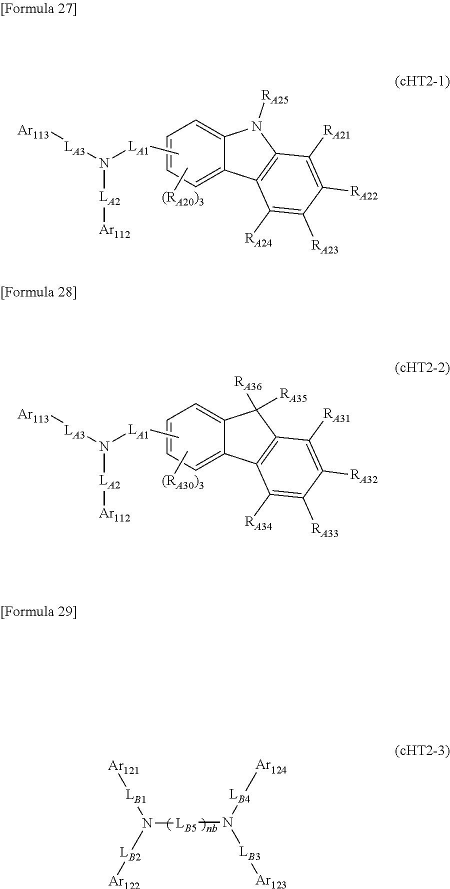

In the formulae (cHT2-1), (CHT2-2), and (cHT2-3):
Ar₁₁₂, Ar₁₁₃, Ar₁₂₁, Ar₁₂₂, Ar₁₂₃, and Ar₁₂₄ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{A1}, L_{A2}, L_{A3}, L_{B1}, L_{B2}, L_{B3}, and L_{B4} are each independently a single bond, a substituted or unsubstituted arylene group having 6 to 50 ring carbon atoms, or a substituted or unsubstituted divalent heterocyclic group having 5 to 50 ring atoms;
nb is 1, 2, 3 or 4;
when nb is 1, L_{B5} is a substituted or unsubstituted arylene group having 6 to 50 ring carbon atoms, or a substituted or unsubstituted divalent heterocyclic group having 5 to 50 ring atoms;
when nb is 2, 3, or 4, a plurality of L_{B5} are mutually the same or different;
when nb is 2, 3, or 4, a plurality of L_{B5} are mutually bonded to form a substituted or unsubstituted monocyclic ring, mutually bonded to form a substituted or unsubstituted fused ring, or not mutually bonded;
L_{B5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms; and
a combination of R_{A35} and R_{A36} are mutually bonded to form a substituted or unsubstituted monocyclic ring, mutually bonded to form a substituted or unsubstituted fused ring, or not mutually bonded;
R_{A25}, and R_{A35} and R_{A36} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R_{A20} to R_{A24} are mutually bonded to form a substituted or unsubstituted monocyclic ring, mutually bonded to form a substituted or unsubstituted fused ring, or not mutually bonded;
at least one combination of adjacent two or more of R_{A30} to R_{A34} are mutually bonded to form a substituted or unsubstituted monocyclic ring, mutually bonded to form a substituted or unsubstituted fused ring, or not mutually bonded;
R_{A20} to R_{A24} and R_{A30} to R_{A34} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
a plurality of R_{A20} are mutually the same or different;
a plurality of R_{A30} are mutually the same or different;
R₉₀₁ to R₉₀₄ in the compounds represented by the formulae (cHT2-1), (cHT2-2), and (cHT2-3) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of Roos are mutually the same or different; and
when a plurality of R₉₀₄ are present, the plurality of R₉₀₄ are mutually the same or different.

In an exemplary arrangement of the organic EL devices of the first, second, and third exemplary embodiments, a first amino group represented by a formula (c21) below and a second amino group represented by a formula (c22) below in the compound represented by the formula (cHT2-3) are an identical group or different groups.

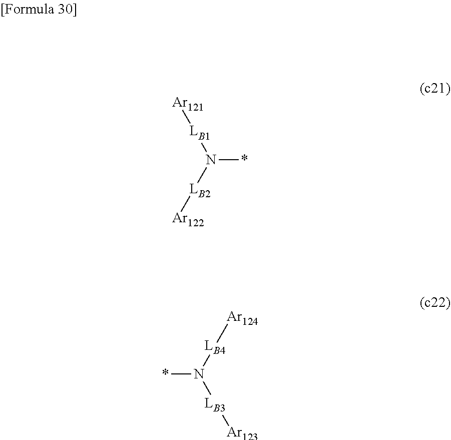

In the formulae (c21) and (c22), * each represent a bonding position to L_{B5}.

In an exemplary arrangement of the organic EL devices of the first, second, and third exemplary embodiments, a substituent for a “substituted or unsubstituted” group in the compounds represented by the formulae (cHT2-1), (CHT2-2), and (cHT2-3) is not a group represented by —N(R_{C6})(R_{C7}).

In an exemplary arrangement of the organic EL devices of the first, second, and third exemplary embodiments, R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of R_{C6} are mutually the same or different, and a plurality of R_{C7} are mutually the same or different.

In an exemplary arrangement of the organic EL devices of the first, second, and third exemplary embodiments, when a substituent for a “substituted or unsubstituted” group is not a group represented by —N(R_{C6})(R_{C7}), the compounds represented by the formulae (cHT2-1) and (cHT2-2) are each a monoamine compound.

In an exemplary arrangement of the organic EL devices of the first, second, and third exemplary embodiments, when a substituent for a “substituted or unsubstituted” group is not a group represented by —N(R_{C6})(R_{C7}), the compound represented by the formulae (cHT2-3) is a diamine compound.

In an exemplary arrangement of the organic EL devices of the first, second, and third exemplary embodiments, the compound represented by the formula (C1) contains at least one compound selected from the group consisting of a compound represented by a formula (cHT3-1), a compound represented by a formula (cHT3-2), a compound represented by a formula (cHT3-3), and a compound represented by a formula (cHT3-4) below.

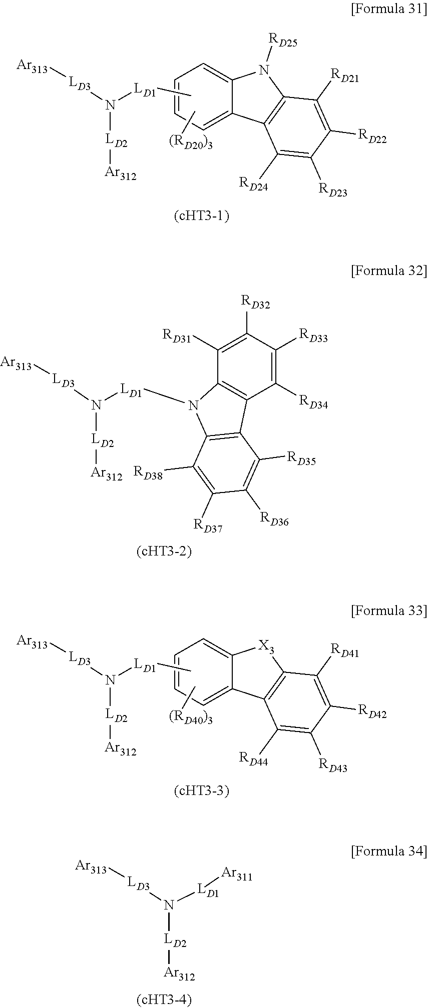

In the formulae (cHT3-1), (cHT3-2), (CHT3-3), and (cHT3-4):
Ar₃₁₁ is a group represented by one of a formula (1-a), a formula (1-b), a formula (1-c), and a formula (1-d) below;
Ar₃₁₂ and Ar₃₁₃ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{D1}, L_{D2}, and Los are each independently a single bond, a substituted or unsubstituted arylene group having 6 to 50 ring carbon atoms, or a substituted or unsubstituted divalent heterocyclic group having 5 to 50 ring atoms;
at least one combination of adjacent two or more of R_{D20} to R_{D24} are mutually bonded to form a substituted or unsubstituted monocyclic ring, mutually bonded to form a substituted or unsubstituted fused ring, or not mutually bonded;
at least one combination of adjacent two or more of R_{D31} to R_{D38} are mutually bonded to form a substituted or unsubstituted monocyclic ring, mutually bonded to form a substituted or unsubstituted fused ring, or not mutually bonded;
at least one combination of adjacent two or more of R_{D40} to R_{D44} are mutually bonded to form a substituted or unsubstituted monocyclic ring, mutually bonded to form a substituted or unsubstituted fused ring, or not mutually bonded;
X₃ is an oxygen atom, a sulfur atom, or C(R_{D45})(R_{D46});
a combination of R_{D45} and R_{D46} are mutually bonded to form a substituted or unsubstituted monocyclic ring, mutually bonded to form a substituted or unsubstituted fused ring, or not mutually bonded;
R_{D25}, and R_{D20} to R_{D24}, R_{D31} to R_{D38}, R_{D40} to R_{D44}, R_{D45} and R_{D46} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
a plurality of R_{D20} are mutually the same or different;
a plurality of R_{D40} are mutually the same or different;
R₉₀₁ to R₉₀₄ in the compounds represented by the formulae (cHT3-1), (cHT3-2), (CHT3-3), and (cHT3-4) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different; and
when a plurality of R₉₀₄ are present, the plurality of R₉₀₄ are mutually the same or different.

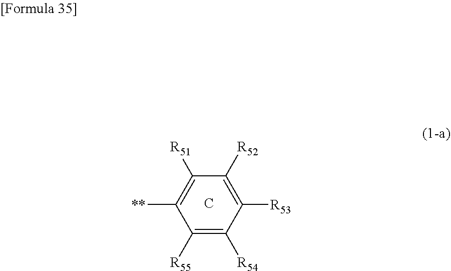

In the formula (1-a):
none of a combination(s) of adjacent two or more of R₅₁ to R₅₅ are bonded to each other;
R₅₁ to R₅₅ are each independently a hydrogen atom or a substituted or unsubstituted alkyl group having 1 to 6 carbon atoms; and
** represents a bonding position to L_{D1}.

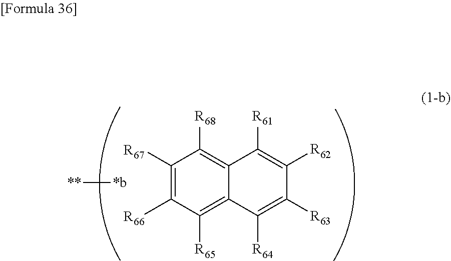

In the formula (1-b):
one of R₆₁ to Res is a single bond with *b;
none of a combination(s) adjacent two or more of R₆₁ to R₆₈ not being the single bond with * b are mutually bonded;
R₆₁ to R₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

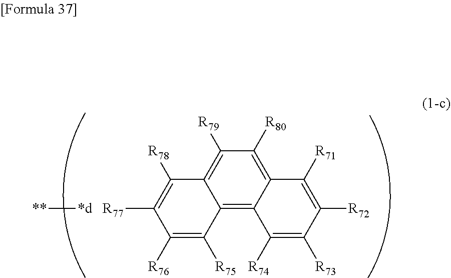

In the formula (1-c):
one of R₇₁ to Rao is a single bond with *d;
none of a combination(s) of adjacent two or more of R₇₁ to Rao not being the single bond with *d are bonded to each other;
R₇₁ to Rao not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

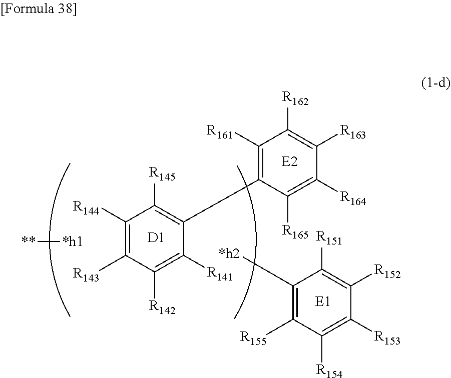

In the formula (1-d):
one of R₁₄₁ to R₁₄₅ is a single bond with *h1, and another one of R₁₄₁ to R₁₄₅ is a single bond with *h2;
none of a combination(s) of adjacent two or more of R₁₄₁ to R₁₄₅ not being the single bond with *h1 and not being the single bond with *h2 are bonded to each other;
at least one combination of adjacent two or more of R₁₅₁ to R₁₅₅ are mutually bonded to form a substituted or unsubstituted monocyclic ring, mutually bonded to form a substituted or unsubstituted fused ring, or not mutually bonded;
at least one combination of adjacent two or more of R₁₆₁ to R₁₆₅ are mutually bonded to form a substituted or unsubstituted monocyclic ring, mutually bonded to form a substituted or unsubstituted fused ring, or not mutually bonded;
R₁₄₁ to R₁₄₅ not being the single bond with *h1 and not being the single bond with *h2 as well as R₁₅₁ to R₁₅₅ and R₁₆₁ to R₁₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

In an exemplary arrangement of the organic EL devices of the first, second, and third exemplary embodiments, a substituent for the “substituted or unsubstituted” group in the compounds represented by the formulae (cHT3-1), (CHT3-2), (cHT3-3), and (cHT3-4) is not a group represented by —N(R_{C6})(R_{C7}), in which R_{C6} and R_{C7} are
each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of Rce are mutually the same or different, and a plurality of R_{C7} are mutually the same or different.

In an exemplary arrangement of the organic EL devices of the first, second, and third exemplary embodiments, when a substituent for a “substituted or unsubstituted” group is not a group represented by —N(R_{C6})(R_{C7}), the compounds represented by the formulae (cHT3-1), (cHT3-2), (cHT3-3), and (cHT3-4) are each a monoamine compound.

In an exemplary arrangement of the organic EL devices of the first, second, and third exemplary embodiments, the second anode-side organic layer contains at least one compound selected from the group consisting of a compound represented by the formula (cHT3-1), a compound represented by the formula (cHT3-2), a compound represented by the formula (cHT3-3), and a compound represented by the formula (cHT3-4).

### First Organic Material

In an exemplary arrangement of the organic EL devices of the first, second, and third exemplary embodiments, the first organic material is at least one compound selected from the group consisting of a compound represented by the formula (C1), a compound represented by the formula (cHT2-1), a compound represented by the formula (cHT2-2), and a compound represented by the formula (cHT2-3).

In an exemplary arrangement of the organic EL devices of the first, second, and third exemplary embodiments, the first organic material is a monoamine compound having one substituted or unsubstituted amino group in a molecule.

In an exemplary arrangement of the organic EL devices of the first, second, and third exemplary embodiments, the first organic material has at least one group selected from the group consisting of a group represented by a formula (2-a), a group represented by a formula (2-b), a group represented by a formula (2-c), a group represented by a formula (2-d), a group represented by a formula (2-e), and a group represented by a formula (2-f) below.

In an exemplary arrangement of the organic EL devices of the first, second, and third exemplary embodiments, the first organic material is at least one compound selected from the group consisting of a compound represented by the formula (cHT2-1), a compound represented by the formula (cHT2-2), and a compound represented by the formula (cHT2-3), and at least one of Ar₁₁₂, Ar₁₁₃, Ar₁₂₁, Ar₁₂₂, Ar₁₂₃, or Ar₁₂₄ in the formulae (cHT2-1), (cHT2-2), and (cHT2-3) has at least one group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

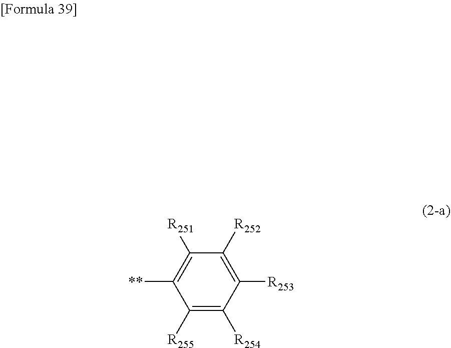

In the formula (2-a):
none of a combination(s) of adjacent two or more of R₂₅₁ to R₂₅₅ are bonded to each other;
R₂₅₁ to R₂₅₅ are each independently a hydrogen atom, or a substituted or unsubstituted alkyl group having 1 to 6 carbon atoms; and
** represents a bonding position.

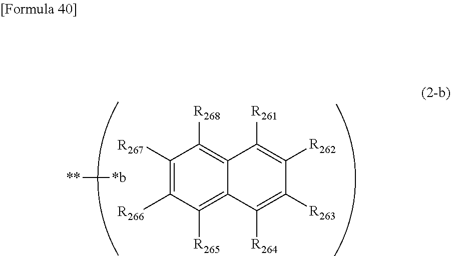

In the formula (2-b):
one of R₂₆₁ to R₂₆₈ is a single bond with *b;
none of a combination(s) of adjacent two or more of R₂₆₁ to R₂₆₈ not being the single bond with *b are bonded to each other;
R₂₆₁ to R₂₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position.

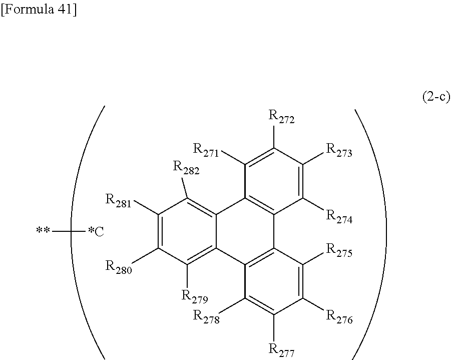

In the formula (2-c):
one of R₂₇₁ to R₂₈₂ is a single bond with *c;
none of a combination(s) of adjacent two or more of R₂₇₁ to R₂₈₂ not being the single bond with *c are bonded to each other;
R₂₇₁ to R₂₈₂ not being the single bond with *c are each independently a hydrogen atom, a substituted or unsubstituted alkyl group having 1 to 6 carbon atoms, or a substituted or unsubstituted aryl group having 6 to 12 ring carbon atoms; and
** represents a bonding position.

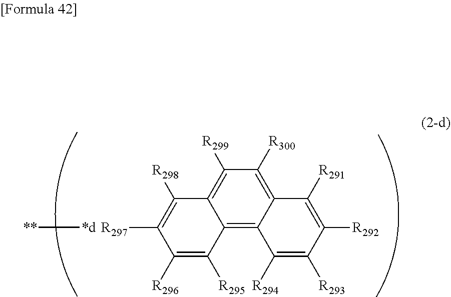

In the formula (2-d):
one of R₂₉₁ to R₃₀₀ is a single bond with *d;
none of a combination(s) of adjacent two or more of R₂₉₁ to R₃₀₀ not being the single bond with *d are bonded to each other;
R₂₉₁ to R₃₀₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position.

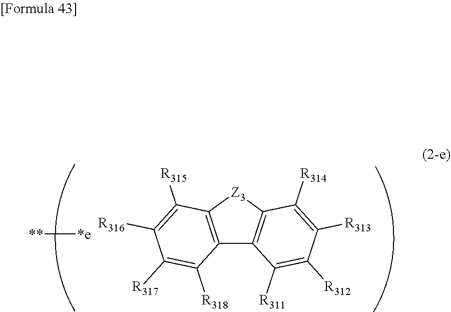

In the formula (2-e):
Z₃ is an oxygen atom, a sulfur atom, NR₃₁₉, or C(R₃₂₀)(R₃₂₁);
one of R₃₁₁ to R₃₂₁ is a single bond with *e, or one of carbon atoms of a substituted or unsubstituted benzene ring below formed by mutually bonding a combination of adjacent two or more of R₃₁₁ to R₃₁₈ is bonded to *e by a single bond;
a combination of adjacent two or more of R₃₁₁ to R₃₁₈ not being the single bond with *e are mutually bonded to form a substituted or unsubstituted benzene ring, or not mutually bonded;
R₃₁₁ to R₃₁₈ not being the single bond with *e and not forming the substituted or unsubstituted benzene ring are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

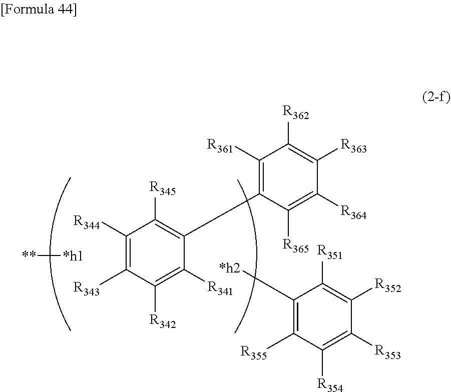

In the formula (2-f):
one of R₃₄₁ to R₃₄₅ is a single bond with *h1, and another one of R₃₄₁ to R₃₄₅ is a single bond with *h2;
none of a combination(s) of adjacent two or more of R₃₄₁ to R₃₄₅ not being the single bond with *h1 and not being the single bond with *h2 are bonded to each other;
at least one combination of adjacent two or more of R₃₅₁ to R₃₅₅ are mutually bonded to form a substituted or unsubstituted monocyclic ring, mutually bonded to form a substituted or unsubstituted fused ring, or not mutually bonded;
at least one combination of adjacent two or more of R₃₆₁ to R₃₆₅ are mutually bonded to form a substituted or unsubstituted monocyclic ring, mutually bonded to form a substituted or unsubstituted fused ring, or not mutually bonded;
R₃₄₁ to R₃₄₅ not being the single bond with *h1 and not being the single bond with *h2 as well as R₃₅₁ to R₃₅₅ and R₃₆₁ to R₃₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

In an exemplary arrangement of the organic EL devices of the first, second, and third exemplary embodiments, a group represented by the formula (2-a), a group represented by the formula (2-b), a group represented by the formula (2-c), a group represented by the formula (2-d), a group represented by the formula (2-e), and a group represented by the formula (2-f) are each independently bonded directly, through a phenylene group, or through a biphenylene group to a nitrogen atom of an amino group of the monoamine compound.

In an exemplary arrangement of the organic EL devices of the first, second, and third exemplary embodiments, the first organic material is a monoamine compound having one substituted or unsubstituted amino group in a molecule, and a group represented by the formula (2-a), a group represented by the formula (2-b), a group represented by the formula (2-c), a group represented by the formula (2-d), a group represented by the formula (2-e), and a group represented by the formula (2-f) are each independently bonded directly, through a phenylene group, or through a biphenylene group to a nitrogen atom of an amino group of the monoamine compound.

In an exemplary arrangement of the organic EL devices of the first, second, and third exemplary embodiments, the first organic material is a compound represented by the formula (cHT2-1) and at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL devices of the first, second, and third exemplary embodiments, the first organic material is a compound represented by the formula (cHT2-2) and at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL devices of the first, second, and third exemplary embodiments, the first organic material is a compound represented by the formula (cHT2-3) and at least one of Ar₁₂₁, Ar₁₂₂, Ar₁₂₃, or Ar₁₂₄ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In the organic EL devices of the first, second, and third exemplary embodiments, when Z₃ in the formula (2-e) is NR₃₁₉, R₃₁₂ or R₃₁₇ is preferably a single bond with *e.

In an exemplary arrangement of the organic EL devices of the first, second, and third exemplary embodiments, the group represented by the formula (2-e) is a group represented by a formula (2-e7) below.

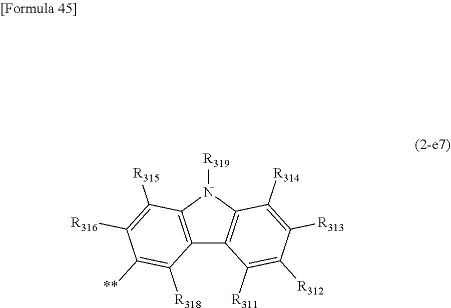

In the formula (2-e7), R₃₁₁ to R₃₁₆, R₃₁₈, and R₃₁₉ respectively represent the same as R₃₁₁ to R₃₁₆, R₃₁₈, and R₃₁₉ in the formula (2-e), and ** represents a bonding position.

In the organic EL devices of the first, second, and third exemplary embodiments, when Z₃ in the formula (2-e) is NR₃₁₉, R₃₁₅, R₃₁₆, or R₃₁₈ is also preferably a single bond with *e.

In an exemplary arrangement of the organic EL devices of the first, second, and third exemplary embodiments, the group represented by the formula (2-e) is a group represented by a formula (2-e4), (2-e5), or (2-e6) below.

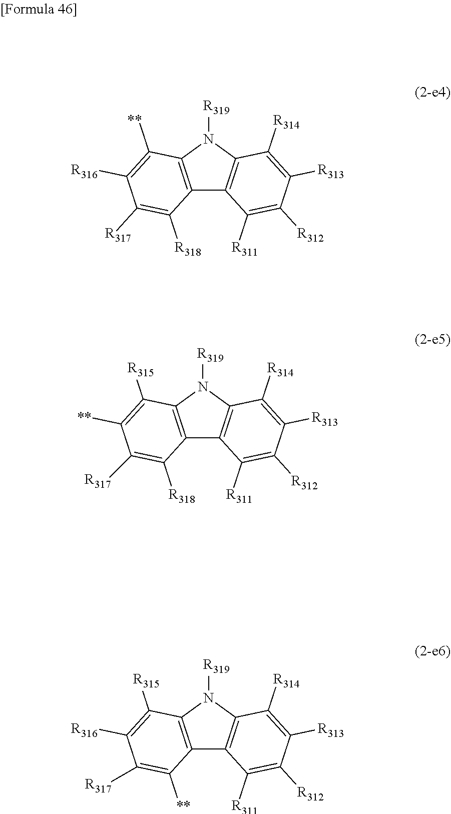

In the formulae (2-e4), (2-e5), and (2-e6), R₃₁₁ to R₃₁₉ respectively represent the same as R₃₁₁ to R₃₁₉ in the formula (2-e), and ** represents a bonding position.

In an exemplary arrangement of the organic EL devices of the first, second, and third exemplary embodiments, the group represented by the formula (2-e) is a group represented by a formula (2-e1), (2-e2), or (2-e3) below.

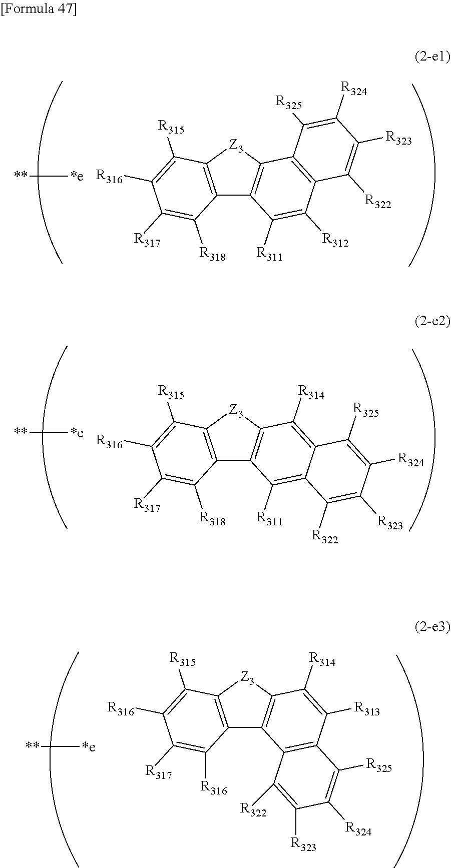

In the formulae (2-e1), (2-e2), and (2-e3):
Z₃ is an oxygen atom, a sulfur atom, NR₃₁₉, or C(R₃₂₀)(R₃₂₁);
one of R₃₁₁ to R₃₂₅ is a single bond with *e;
R₃₁₁ to R₃₁₈ and R₃₂₂ to R₃₂₅ not being the single bond with *e are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

In an exemplary arrangement of the organic EL devices of the first, second, and third exemplary embodiments, ** in the formulae (2-a), (2-b), (2-c), (2-d), (2-e), (2-f), (2-e1), (2-e2), and (2-e3) are each independently a bonding position to L_{A2}, L_{A3}, L_{B1}, L_{B2}, L_{B3}, or L_{B4} or a bonding position to a nitrogen atom of an amino group.

In an exemplary arrangement of the organic EL devices of the first, second, and third exemplary embodiments, the first organic material is a compound having no thiophene ring in a molecule.

In an exemplary arrangement of the organic EL devices of the first, second, and third exemplary embodiments, the refractive index of the first organic material n₁ is 1.8 or more. When the refractive index of the first organic material is increased, the refractive index of the first anode-side organic layer is increased and the refractive index difference between the first anode-side organic layer and the second anode-side organic layer is increased to improve the light extraction efficiency.

In an exemplary arrangement of the organic EL devices of the first, second, and third exemplary embodiments, an ionization potential of the first organic material is 5.7 eV or less.

Herein, the ionization potential of a compound is measured using a photoelectron spectrometer under atmosphere. Specifically, the ionization potential of a compound can be measured by the method described in Examples.

### Third Organic Material

In an exemplary arrangement of the organic EL devices of the first, second, and third exemplary embodiments, the third organic material is at least one compound selected from the group consisting of a compound represented by the formula (cHT3-1), a compound represented by the formula (cHT3-2), a compound represented by the formula (cHT3-3), and a compound represented by the formula (cHT3-4).

In an exemplary arrangement of the organic EL devices of the first, second, and third exemplary embodiments, the third organic material is a monoamine compound.

In an exemplary arrangement of the organic EL devices of the first, second, and third exemplary embodiments, the third organic material is a compound having no thiophene ring in a molecule.

In an exemplary arrangement of the organic EL devices of the first, second, and third exemplary embodiments, the refractive index of the third organic material is 1.85 or less.

In an exemplary arrangement of the organic EL devices of the first, second, and third exemplary embodiments, the refractive index of the third organic material is 1.80 or less.

### Sixth Organic Material

In an exemplary arrangement of the organic EL devices of the first, second, and third exemplary embodiments, the sixth organic material is an amine compound having at least one substituted or unsubstituted amino group in a molecule.

In an exemplary arrangement of the organic EL devices of the first, second, and third exemplary embodiments, the sixth organic material and the third organic material are mutually different compounds.

In an exemplary arrangement of the organic EL devices of the first, second, and third exemplary embodiments, the sixth organic material is a monoamine compound.

In an exemplary arrangement of the organic EL devices of the first, second, and third exemplary embodiments, the sixth organic material is at least one compound selected from the group consisting of a compound represented by the formula (cHT3-1), a compound represented by the formula (cHT3-2), a compound represented by the formula (cHT3-3), and a compound represented by the formula (cHT3-4).

In an exemplary arrangement of the organic EL devices of the first, second, and third exemplary embodiments, the sixth organic material is a compound having no thiophene ring in a molecule.

### Seventh Organic Material

In an exemplary arrangement of the organic EL devices of the first, second, and third exemplary embodiments, the seventh organic material and the third organic material are mutually different compounds.

In an exemplary arrangement of the organic EL devices of the first, second, and third exemplary embodiments, the seventh organic material, the first organic material, the second organic material, the third organic material, the fourth material, the fifth material, and the sixth organic material are mutually different compounds.

In an exemplary arrangement of the organic EL devices of the first, second, and third exemplary embodiments, the fourth anode-side organic layer contains a compound represented by the formula (C1) or a compound represented by the formula (C3).

In an exemplary arrangement of the organic EL devices of the first, second, and third exemplary embodiments, although the third anode-side organic layer and the fourth anode-side organic layer may each contain a compound represented by the formula (C1), the compound contained in the third anode-side organic layer and the compound contained in the fourth anode-side organic layer are mutually different in molecular structure. The compound represented by the formula (C1) is preferably a monoamine compound.

In an exemplary arrangement of the organic EL devices of the first, second, and third exemplary embodiments, in a case where the fourth anode-side organic layer is an electron blocking layer, a compound contained in the electron blocking layer (the seventh organic material) is, for instance, a known compound used for the electron blocking layer and is at least one compound selected from the group consisting of an aromatic amine compound and a carbazole derivative. The compound contained in the electron blocking layer may be a monoamine compound. The compound contained in the electron blocking layer may be a compound having a substituted or unsubstituted carbazolyl group and one substituted or unsubstituted amino group in a molecule.

In an exemplary arrangement of the organic EL devices of the first, second, and third exemplary embodiments, the seventh organic material is a compound having no thiophene ring in a molecule.

In an exemplary arrangement of the organic EL devices of the first, second, and third exemplary embodiments, the first anode-side organic layer, the second anode-side organic layer, the third anode-side organic layer, and the fourth anode-side organic layer contain one or more compounds different from each other.

In an exemplary arrangement of the organic EL devices of the first, second, and third exemplary embodiments, the first anode-side organic layer, the second anode-side organic layer, the third anode-side organic layer, and the fourth anode-side organic layer each independently contain a monoamine compound having only one substituted or unsubstituted amino group in a molecule.

In an exemplary arrangement of the organic EL devices of the first, second, and third exemplary embodiments, the first anode-side organic layer, the second anode-side organic layer, the third anode-side organic layer, and the fourth anode-side organic layer contain no diamine compound.

In an exemplary arrangement of the organic EL devices of the first, second, and third exemplary embodiments, at least one of the first anode-side organic layer, the second anode-side organic layer, the third anode-side organic layer, or the fourth anode-side organic layer may also contain a diamine compound. The compound represented by the formula (C3) is preferably a diamine compound.

In the organic EL devices of the first, second, and third exemplary embodiments, R₉₀₁, R₉₀₂, R₉₀₃, and R₉₀₄ in a compound contained in each layer of the first hole transporting zone are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

In the first, second, and third exemplary embodiment, the groups specified to be “substituted or unsubstituted” are each preferably an “unsubstituted” group.

### Method of Producing Hole Transporting Zone Material

The hole transporting zone material according to the first, second, and third exemplary embodiments can be produced by a known method or through a known alternative reaction using a known material(s) tailored for the target compound in accordance with the known method.

### Specific Examples of Hole Transporting Zone Material

Specific examples of the hole transporting zone material according to the first, second, and third exemplary embodiments include the compounds listed in, for instance, the “common arrangement (3) among all the exemplary embodiments” described later.

### Second Organic Material

In an exemplary arrangement of the organic EL devices of the first, second, and third exemplary embodiments, the second organic material is an acceptor material.

In an exemplary arrangement of the organic EL devices of the first, second, and third exemplary embodiments, the second organic material has at least one of a first cyclic structure represented by a formula (P11) below or a second cyclic structure represented by a formula (P12) below.

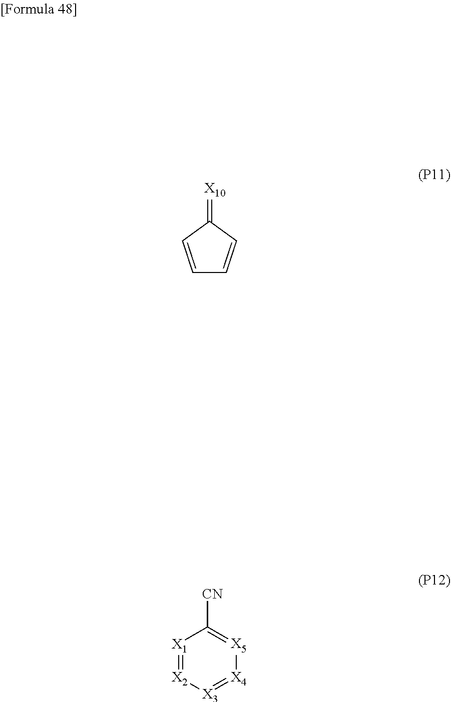

The first cyclic structure represented by the formula (P11) is fused, in a molecule of the second organic material, to at least one cyclic structure of a substituted or unsubstituted aromatic hydrocarbon ring having 6 to 50 ring carbon atoms or a substituted or unsubstituted heterocycle having 5 to 50 ring atoms, and a structure represented by =X₁₀ is represented by a formula (11a), (11b), (11c), (11d), (11e), (11f), (11g), (11h), (11i), (11j), (11k) or (11m) below.

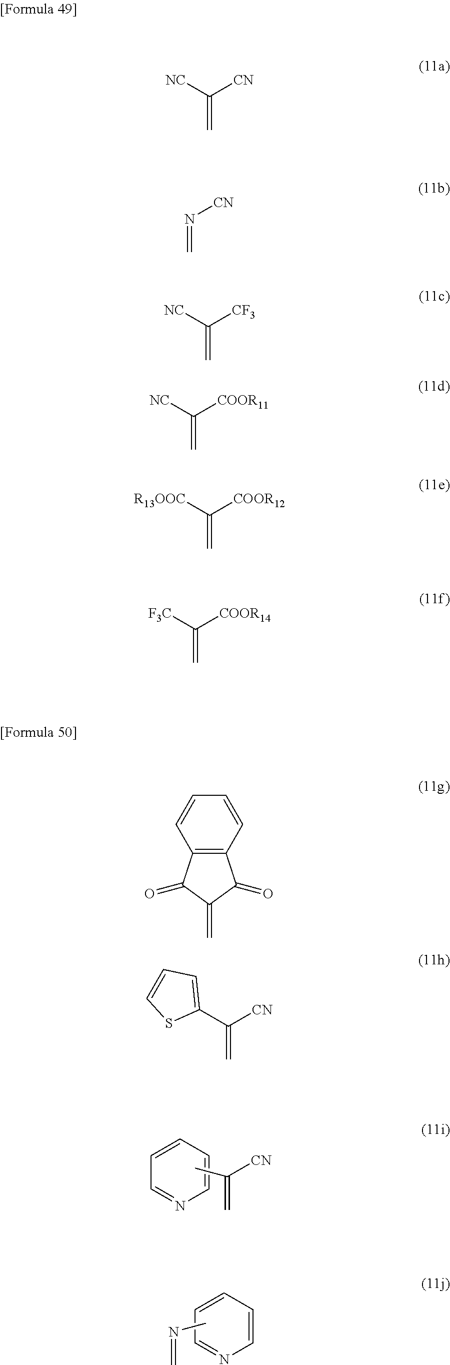

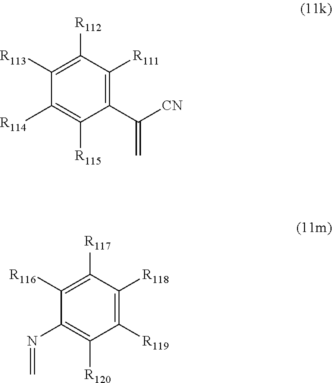

In the formula (11a), (11b), (11c), (11d), (11e), (11f), (11g), (11h), (11i), (11j), (11k) or (11m):
R₁₁ to R₁₄ and R₁₁₁ to R₁₂₀ are each independently a hydrogen atom, a halogen atom, a hydroxy group, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms.

In the formula (P12):
X₁ to X₅ are each independently a nitrogen atom, a carbon atom bonded to R₁₅, or a carbon atom bonded to another atom in a molecule of the second organic material;
at least one of X₁ to X₅ is a carbon atom bonded to another atom in the molecule of the second organic material;
R₁₅ is selected from the group consisting of a hydrogen atom, a halogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted alkenyl group having 2 to 50 carbon atoms, a substituted or unsubstituted aralkyl group having 7 to 50 carbon atoms, a carboxy group, a substituted or unsubstituted ester group, a substituted or unsubstituted carbamoyl group, a nitro group, and a substituted or unsubstituted siloxanyl group; and
when a plurality of R₁₅ are present, the plurality of R₁₅ are mutually the same or different.

In the second organic material, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL devices of the first, second, and third exemplary embodiments, the second organic material has at least one cyano group.

In an exemplary arrangement of the organic EL devices of the first, second, and third exemplary embodiments, the first anode-side organic layer contains the first organic material. The second organic material and the first organic material are mutually different.

In an exemplary arrangement of the organic EL devices of the first, second, and third exemplary embodiments, a content of the second organic material in the first anode-side organic layer is less than 50 mass %.

In an exemplary arrangement of the organic EL devices of the first, second, and third exemplary embodiments, the content of the second organic material in the first anode-side organic layer is 10 mass % or less or 5 mass % or less.

In an exemplary arrangement of the organic EL devices of the first, second, and third exemplary embodiments, the content of the second organic material in the first anode-side organic layer is in a range from 1 mass % to 3 mass %.

In an exemplary arrangement of the organic EL devices of the first, second, and third exemplary embodiments, in a case where the first anode-side organic layer contains the second organic material and the first organic material, a content of the first organic material in the first anode-side organic layer is preferably 40 mass % or more, more preferably 45 mass % or more, and still more preferably 50 mass % or more. The content of the first organic material in the first anode-side organic layer is preferably 99.5 mass % or less. The total of the content of the second organic material and the content of the first organic material in the first anode-side organic layer is 100 mass % or less.

An ester group herein is at least one group selected from the group consisting of an alkyl ester group and an aryl ester group.

An alkyl ester group herein is represented, for instance, by —C(═O) OR^{E}. R^{E} is exemplified by a substituted or unsubstituted alkyl group having 1 to 50 carbon atoms (preferably 1 to 10 carbon atoms).

An aryl ester group herein is represented, for instance, by —C(═O) OR^{Ar}. R^{Ar} is exemplified by a substituted or unsubstituted aryl group having 6 to 30 ring carbon atoms.

A siloxanyl group herein, which is a silicon compound group through an ether bond, is exemplified by a trimethylsiloxanyl group.

A carbamoyl group herein is represented by —CONH₂.

A substituted carbamoyl group herein is represented, for instance, by —

CONH—Ar^{C} or —CONH—R^{C}. Ar^{C} is, for instance, at least one group selected from the group consisting of a substituted or unsubstituted aryl group having 6 to 50 ring carbon atoms (preferably 6 to 10 ring carbon atoms) and a heterocyclic group having 5 to 50 ring atoms (preferably 5 to 14 ring atoms). Ar^{C} may be a group in which a substituted or unsubstituted aryl group having 6 to 50 ring carbon atoms is bonded to a substituted or unsubstituted heterocyclic group having 5 to 50 ring atoms.

R^{C} is exemplified by a substituted or unsubstituted alkyl group having 1 to 50 carbon atoms (preferably 1 to 6 carbon atoms).

In the second organic material, the groups specified to be “substituted or unsubstituted” are each preferably an “unsubstituted” group.

### Specific Examples of Second Organic Material

Specific examples of the second organic material include the compounds listed in, for instance, the “common arrangement (3) among all the exemplary embodiments” described later.

### Additive Material

In an exemplary arrangement of the organic EL devices of the first, second, and third exemplary embodiments, the fourth material and the fifth material as the additive material are each independently an organic material or a metal atom-containing material.

In an exemplary arrangement of the organic EL devices of the first, second, and third exemplary embodiments, the fourth material as the additive material is a material that causes the refractive index of the first anode-side organic layer to satisfy both of the numerical formulae (Numerical Formula N1 and Numerical Formula N2).

In an exemplary arrangement of the organic EL devices of the first, second, and third exemplary embodiments, the fifth material as the additive material is a material that causes the refractive index of the second anode-side organic layer to satisfy both of the numerical formulae (Numerical Formula N1 and Numerical Formula N3).

In an exemplary arrangement of the organic EL devices of the first, second, and third exemplary embodiments, the refractive index of the fifth material is 1.80 or less.

In an exemplary arrangement of the organic EL devices of the first, second, and third exemplary embodiments, the refractive index of the fifth material is 1.78 or less.

In an exemplary arrangement of the organic EL devices of the first, second, and third exemplary embodiments, the additive material is an organic material.

In an exemplary arrangement of the organic EL devices of the first, second, and third exemplary embodiments, the organic material as the additive material is an amine compound having at least one substituted or unsubstituted amino group in a molecule, or a non-amine compound not having a substituted or unsubstituted amino group in a molecule.

In an exemplary arrangement of the organic EL devices of the first, second, and third exemplary embodiments, the organic material as the additive material is an amine compound having a thiophene ring.

In an exemplary arrangement of the organic EL devices of the first, second, and third exemplary embodiments, in a case where an amine compound as the additive material is contained in the anode-side organic layers of the first hole transporting zone, a content of the amine compound in each of the anode-side organic layers is preferably 10 mass % or more, more preferably 20 mass % or more.

In an exemplary arrangement of the organic EL devices of the first, second, and third exemplary embodiments, in a case where the amine compound as the additive material is contained in the anode-side organic layers of the first hole transporting zone, the content of the amine compound in each of the anode-side organic layers is preferably 50 mass % or less.

In an exemplary arrangement of the organic EL devices of the first, second, and third exemplary embodiments, in a case where a non-amine compound as the additive material is contained in the anode-side organic layers of the first hole transporting zone, a content of the non-amine compound in each of the anode-side organic layers is preferably 5 mass % or more, more preferably 10 mass % or more, and still more preferably 20 mass % or more.

In an exemplary arrangement of the organic EL devices of the first, second, and third exemplary embodiments, in a case where the non-amine compound as the additive material is contained in the anode-side organic layers of the first hole transporting zone, the content of the non-amine compound in each of the anode-side organic layers is preferably 50 mass % or less.

Specific examples of the fourth material as the additive material in an exemplary arrangement of the organic EL devices of the first, second, and third exemplary embodiments include the compounds listed in, for instance, the “common arrangement (3) among all the exemplary embodiments” described later.

In an exemplary arrangement of the organic EL devices of the first, second, and third exemplary embodiments, the additive material is a metal atom-containing material.

In an exemplary arrangement of the organic EL devices of the first, second, and third exemplary embodiments, the metal atom-containing material is exemplified by a compound having a perovskite structure (perovskite compound). In an exemplary arrangement of the organic EL devices of the first, second, and third exemplary embodiments, a perovskite compound as the metal atom-containing material is represented by a formula ABX₃ in which A and B are each a cation and X is an anion. For instance, A is CH₃NH₃ (“CH₃NH₃” is sometimes referred to as “MA” hereinafter), CH(NH₂)₂, Cs, or a mixture thereof; B is Pb, Sn, or a mixture thereof; and X is Cl, Br, I, or a mixture thereof. The perovskite compound as the metal atom-containing material is exemplified by CH₃NH₃PbBr₃. A refractive index of CH₃NH₃PbBr₃ at 460 nm is 2.3.

In an exemplary arrangement of the organic EL devices of the first, second, and third exemplary embodiments, the metal atom-containing material is a metal oxide. In an exemplary arrangement of the organic EL devices of the first, second, and third exemplary embodiments, the metal oxide as the metal atom-containing material is exemplified by zinc oxide (ZnO).

In an exemplary arrangement of the organic EL devices of the first, second, and third exemplary embodiments, the fourth material as the additive material is a perovskite compound or zinc oxide (ZnO).

Specific examples of the fifth material as the additive material in an exemplary arrangement of the organic EL devices of the first, second, and third exemplary embodiments include the compounds listed in, for instance, the “common arrangement (3) among all the exemplary embodiments” described later.

The description on the common arrangement (1) among the first exemplary embodiment, the second exemplary embodiment, and the third exemplary embodiment has been thus made above.

### Fourth Exemplary Embodiment

An organic electroluminescence device according to a fourth exemplary embodiment includes a cathode, an anode, a first emitting region provided between the cathode and the anode, and a first hole transporting zone provided between the anode and the first emitting region, in which the first emitting region includes at least one emitting layer, the first hole transporting zone includes at least a first anode-side organic layer, a second anode-side organic layer, and a third anode-side organic layer, the first anode-side organic layer, the second anode-side organic layer, and the third anode-side organic layer are disposed in this order from a side close to the anode between the anode and the first emitting region, the second anode-side organic layer contains a second organic material, the second organic material is an amine compound having at least one substituted or unsubstituted amino group in a molecule, the third anode-side organic layer has a film thickness of 20 nm or more, the third anode-side organic layer contains a third organic material, the third organic material is an amine compound having at least one substituted or unsubstituted amino group in a molecule, at least one of the second anode-side organic or the third anode-side organic layer contains an additive material, the additive material contained in the second anode-side organic layer is a fourth material, the additive material contained in the third anode-side organic layer is a fifth material, the fourth material and the fifth material are each independently an organic material or a metal atom-containing material, the second organic material, the third organic material, the fourth material, and the fifth material are mutually different, and the second anode-side organic layer and the third anode-side organic layer satisfy a numerical formula (Numerical Formula NX1) and at least one of numerical formulae (Numerical Formula NX2 and Numerical Formula NX3) below.

$${NM}_{2} > {NM}_{3} \qquad ( Numerical Formula N X1 )$$

$${NM}_{24} > N {M}_{20} \qquad ( Numerical Formula N X2 )$$

$${NM}_{30} > N {M}_{35} \qquad ( Numerical Formula N X3 )$$

In the formulae (Numerical Formula NX1), (Numerical Formula NX2), and (Numerical Formula NX3):
NM₂ represents a refractive index of a constituent material contained in the second anode-side organic layer;
NM₃ represents a refractive index of a constituent material contained in the third anode-side organic layer;
NM₂₀ represents a refractive index of the constituent material contained in the second anode-side organic layer not containing the fourth material;
NM₂₄ represents a refractive index of the constituent material contained in the second anode-side organic layer containing the fourth material;
NM₃₀ represents a refractive index of the constituent material contained in the third anode-side organic layer not containing the fifth material; and
NM₃₅ represents a refractive index of the constituent material contained in the third anode-side organic layer containing the fifth material.

According to the fourth exemplary embodiment, the device performance of the organic EL device is improvable. In the organic EL device of the fourth exemplary embodiment, the refractive index NM₂ of the constituent material contained in the second anode-side organic layer is larger than the refractive index NM₃ of the constituent material contained in the third anode-side organic layer. Light extraction efficiency of the organic EL device is improved by the refractive index NM₂ being larger than the refractive index NM₃. Further, in the organic EL device according to the fourth exemplary embodiment, at least one of the second anode-side organic layer or the third anode-side organic layer contains an additive material satisfying at least one of the formulae (Numerical Formula NX2 and Numerical Formula NX3). Therefore, according to the fourth exemplary embodiment, a difference NM₂-NM₃ in refractive index between the second anode-side organic layer and the third anode-side organic layer is increasable to further improve the light extraction efficiency of the organic EL device. The organic EL device of the fourth exemplary embodiment improves luminous efficiency as the device performance as compared with an organic EL device having a hole transporting zone containing no additive material. An exemplary arrangement of the organic EL device of the fourth exemplary embodiment prolongs a lifetime as compared with an organic EL device having a hole transporting zone containing no additive material.

When the second anode-side organic layer contains a single type of compound, the refractive index NM₂ of the constituent material contained in the second anode-side organic layer corresponds to a refractive index of the single type of compound. When the second anode-side organic layer contains a plurality of types of compounds, the refractive index NM₂ of the constituent material contained in the second anode-side organic layer corresponds to a refractive index of a mixture containing the plurality types of compounds. The refractive index NM₁, the refractive index NM₃, and refractive indices of constituent materials of other layers are defined in the same manner as the refractive index NM₂. The refractive index can be measured by the measurement method described in Examples below. Herein, a value of the refractive index at 2.7 eV in the substrate parallel direction (Ordinary direction) measured by multi-incidence angle spectroscopic ellipsometry measurement is defined as a refractive index of the measurement target material. The refractive index at 2.7 eV corresponds to a refractive index at 460 nm.

In a “common arrangement (2) among the fourth exemplary embodiment, the fifth exemplary embodiment, and the sixth exemplary embodiment” and the “common arrangement (3) among all the exemplary embodiments” which are later described, the organic EL device of the fourth exemplary embodiment will be further described.

### Fifth Exemplary Embodiment

An organic electroluminescence device according to a fifth exemplary embodiment includes a cathode, an anode, a first emitting region provided between the cathode and the anode, and a first hole transporting zone provided between the anode and the first emitting region, in which the first emitting region includes at least one emitting layer, the first hole transporting zone includes at least a first anode-side organic layer and a second anode-side organic layer, the first anode-side organic layer and the second anode-side organic layer are disposed in this order from a side close to the anode between the anode and the first emitting region, the second anode-side organic layer contains a second organic material, the second organic material is an amine compound having at least one substituted or unsubstituted amino group in a molecule, the second anode-side organic layer contains an additive material, the additive material contained in the second anode-side organic layer is a fourth material, and a difference n₄−n₂ between a refractive index of the fourth material n₄ and a refractive index of the second organic material n₂ is 0.05 or more.

According to the fifth exemplary embodiment, the device performance of the organic EL device is improvable. In the organic EL device according to the fifth exemplary embodiment, since the refractive index difference n₄−n₂ is 0.05 or more, the fourth material as the additive material has a higher refractive index than the second organic material. By containing the fourth material in the second anode-side organic layer, the refractive index difference between the second anode-side organic layer and the third anode-side organic layer is increasable to further improve the light extraction efficiency of the organic EL device. Similar to the organic EL device of the fourth exemplary embodiment, the organic EL device of the fifth exemplary embodiment improves the luminous efficiency as the device performance as compared with an organic EL device having a hole transporting zone containing no additive material. An exemplary arrangement of the organic EL device of the fifth exemplary embodiment prolongs a lifetime as compared with an organic EL device having a hole transporting zone containing no additive material.

In an exemplary arrangement of the organic EL device of the fifth exemplary embodiment, the first hole transporting zone includes a third anode-side organic layer between the second anode-side organic layer and the first emitting region.

In an exemplary arrangement of the organic EL device of the fifth exemplary embodiment, the first anode-side organic layer, the second anode-side organic layer, and the third anode-side organic layer are disposed between the anode and the first emitting region in this order from a side close the anode.

In an exemplary arrangement of the organic EL device of the fifth exemplary embodiment, the third anode-side organic layer has a film thickness of 20 nm or more.

In an exemplary arrangement of the organic EL device of the fifth exemplary embodiment, the third anode-side organic layer contains a third organic material.

In an exemplary arrangement of the organic EL device of the fifth exemplary embodiment, the third organic material is an amine compound having at least one substituted or unsubstituted amine group in a molecule.

In an exemplary arrangement of the organic EL device of the fifth exemplary embodiment, the third anode-side organic layer contains an additive material. The additive material contained in the third anode-side organic layer is a fifth material.

In an exemplary arrangement of the organic EL device of the fifth exemplary embodiment, the fourth material and the fifth material are each independently an organic material or a metal atom-containing material.

In an exemplary arrangement of the organic EL device of the fifth exemplary embodiment, the second organic material, the third organic material, the fourth material, and the fifth material are mutually different.

In an exemplary arrangement of the organic EL device according to the fifth exemplary embodiment, the second anode-side organic layer and the third anode-side organic layer satisfy the numerical formula (Numerical Formula NX1) and at least one of the formulae (Numerical Formula NX2 and Numerical Formula NX3) of the fourth exemplary embodiment.

In the “common arrangement (2) among the fourth exemplary embodiment, the fifth exemplary embodiment, and the sixth exemplary embodiment” and the “common arrangement (3) among all the exemplary embodiments” which are later described, the organic EL device of the fifth exemplary embodiment will be further described.

### Sixth Exemplary Embodiment

An organic electroluminescence device according to a sixth exemplary embodiment includes a cathode, an anode, a first emitting region provided between the cathode and the anode, and a first hole transporting zone provided between the anode and the first emitting region, in which the first emitting region includes at least one emitting layer, the first hole transporting zone includes at least a first anode-side organic layer, a second anode-side organic layer, and a third anode-side organic layer, the first anode-side organic layer, the second anode-side organic layer, and the third anode-side organic layer are disposed in this order from a side close to the anode between the anode and the first emitting region, the third anode-side organic layer contains a third organic material and an additive material, the additive material contained in the third anode-side organic layer is a fifth material, and a difference n₃−n₅ between a refractive index of the third organic material n₃ and a refractive index of the fifth material n₅ is 0.02 or more.

According to the sixth exemplary embodiment, the device performance of the organic EL device is improvable. In the organic EL device of the sixth exemplary embodiment, since the refractive index difference n₃−n₅ is 0.02 or more, the fifth material as the additive material has a lower refractive index than the third organic material. By containing the fifth material in the third anode-side organic layer, the refractive index difference between the second anode-side organic layer and the third anode-side organic layer is increasable to further improve the light extraction efficiency of the organic EL device. Similar to the organic EL devices of the fourth exemplary embodiment and the fifth exemplary embodiment, the organic EL device of the sixth exemplary embodiment improves the luminous efficiency as the device performance as compared with an organic EL device having a hole transporting zone containing no additive material. An exemplary arrangement of the organic EL device of the sixth exemplary embodiment prolongs a lifetime as compared with an organic EL device having a hole transporting zone containing no additive material.

In an exemplary arrangement of the organic EL device according to the sixth exemplary embodiment, a difference n₃−n₅ between the refractive index of the third organic material n₃ and the refractive index of the fifth material n₅ is 0.03 or more.

In an exemplary arrangement of the organic EL device of the sixth exemplary embodiment, the third anode-side organic layer has a film thickness of 20 nm or more.

In an exemplary arrangement of the organic EL device of the sixth exemplary embodiment, the third organic material is an amine compound having at least one substituted or unsubstituted amine group in a molecule.

In an exemplary arrangement of the organic EL device of the sixth exemplary embodiment, the second anode-side organic layer contains a second organic material.

In an exemplary arrangement of the organic EL device of the sixth exemplary embodiment, the second organic material is an amine compound having at least one substituted or unsubstituted amine group in a molecule.

In an exemplary arrangement of the organic EL device of the sixth exemplary embodiment, the second anode-side organic layer contains an additive material. The additive material contained in the second anode-side organic layer is a fourth material.

In an exemplary arrangement of the organic EL device of the sixth exemplary embodiment, the fourth material and the fifth material are each independently an organic material or a metal atom-containing material.

In an exemplary arrangement of the organic EL device of the sixth exemplary embodiment, the second organic material, the third organic material, the fourth material, and the fifth material are mutually different.

In an exemplary arrangement of the organic EL device according to the sixth exemplary embodiment, the second anode-side organic layer and the third anode-side organic layer satisfy the numerical formula (Numerical Formula NX1) and at least one of the formulae (Numerical Formula NX2 and Numerical Formula NX3) of the fourth exemplary embodiment.

In the “common arrangement (2) among the fourth exemplary embodiment, the fifth exemplary embodiment, and the sixth exemplary embodiment” and the “common arrangement (3) among all the exemplary embodiments” which are later described, the organic EL device of the sixth exemplary embodiment will be further described.

### Common Arrangement (2) Among Fourth Exemplary Embodiment, Fifth Exemplary Embodiment, and Sixth Exemplary Embodiment

Regarding the common arrangement (2) among the fourth exemplary embodiment, the fifth exemplary embodiment, and the sixth exemplary embodiment, an arrangement applicable to each of the fourth exemplary embodiment, the fifth exemplary embodiment, and the sixth exemplary embodiment described herein will be described below.

### Hole Transporting Zone

Herein, a region disposed between the anode and the first emitting region and including a plurality of organic layers is referred to as a first hole transporting zone.

The first hole transporting zone of each of the organic EL devices of the fourth, fifth, and sixth exemplary embodiments includes at least the first-side organic layer and the second anode-side organic layer.

The first hole transporting zone of the organic EL device of the fourth exemplary embodiment includes at least the first-side organic layer, the second anode-side organic layer, and the third anode-side organic layer. The first anode-side organic layer, the second anode-side organic layer, and the third anode-side organic layer are disposed between the anode and the first emitting region in this order from a side close the anode.

In an exemplary arrangement of the organic EL devices of the fourth, fifth, and sixth exemplary embodiments, the anode is in direct contact with the first anode-side organic layer.

In an exemplary arrangement of the organic EL devices of the fourth, fifth, and sixth exemplary embodiments, the first-side organic layer is in direct contact with the second anode-side organic layer.

In an exemplary arrangement of the organic EL devices of the fourth, fifth, and sixth exemplary embodiments, the second anode-side organic layer is in direct contact with the third anode-side organic layer.

In an exemplary arrangement of the organic EL devices of the fourth, fifth, and sixth exemplary embodiments, the third anode-side organic layer is in direct contact with the first emitting region.

In an exemplary arrangement of the organic EL devices of the fourth, fifth, and sixth exemplary embodiments, a difference NM₂−NM₃ between a refractive index NM₂ of a constituent material contained in the second anode-side organic layer and a refractive index NM₃ of a constituent material contained in the third anode-side organic layer satisfies a relationship of a numerical formula (Numerical Formula NX4) below.

$${NM}_{2} - {NM}_{3} \geq 0 \text{.05} \qquad ( Numerical Formula N X4 )$$

In an exemplary arrangement of the organic EL devices of the fourth, fifth, and sixth exemplary embodiments, the refractive index difference NM₂−NM₃ satisfies a relationship of a numerical formula (Numerical Formula NX41, Numerical Formula NX42, or Numerical Formula NX43) below.

$${NM}_{2} - {NM}_{3} \geq 0.1 \qquad ( Numerical Formula N X41 )$$

$${NM}_{2} - {NM}_{3} \geq 0.15 \qquad ( Numerical Formula N X42 )$$

$${NM}_{2} - {NM}_{3} \geq 0.2 \qquad ( Numerical Formula N X43 )$$

In an exemplary arrangement of the organic EL devices of the fourth, fifth, and sixth exemplary embodiments, the second anode-side organic layer does not contain the compound contained in the third anode-side organic layer. An arrangement satisfying the above condition is, for instance, as follows: when a compound CA, a compound CB, a compound AA and a compound AB are mutually different compounds, the third anode-side organic layer contains a single type of compound (i.e., the compound AA) and the second anode-side organic layer contains two types of compounds (i.e., the compound CA and the compound CB). Further, the above condition is also satisfied when the third anode-side organic layer contains two types of compounds (i.e., the compound AA and the compound AB) and the second anode-side organic layer contains two types of compounds (i.e., the compound CA and the compound CB). On the other hand, the above condition is not satisfied, for instance, when the third anode-side organic layer contains a single type of compound (i.e., the compound AA) and the second anode-side organic layer contains two types of compounds (i.e., the compound CA and the compound AA), because the second anode-side organic layer and the third anode-side organic layer contain the identical compound (the compound AA).

In an exemplary arrangement of the organic EL devices of the fourth, fifth, and sixth exemplary embodiments, the first-side organic layer and the second anode-side organic layer contain at least one identical compound. An arrangement satisfying the above condition is, for instance, as follows: when the first anode-side organic layer contains two types of compounds (i.e., the compound AA and the compound AB) and the second anode-side organic layer contains two types of compounds (i.e., the compound CA and the compound AA), because the first anode-side organic layer and the second anode-side organic layer contain the identical compound (the compound AA).

In an exemplary arrangement of the organic EL devices of the fourth, fifth, and sixth exemplary embodiments, the second anode-side organic layer contains the second organic material and the fourth material.

In an exemplary arrangement of the organic EL devices of the fourth, fifth, and sixth exemplary embodiments, the refractive index NM₂₄ is larger than the refractive index NM₂₀ as shown in the numerical formula (Numerical Formula NX2). By containing the fourth material as the additive material in the second anode-side organic layer, the refractive index of the second anode-side organic layer is increased. As a result, the difference NM₂−NM₃ in refractive index between the second anode-side organic layer and the third anode-side organic layer is easily increased.

In an exemplary arrangement of the organic EL devices of the fourth, fifth, and sixth exemplary embodiments, the difference n₄−n₂ between the refractive index of the fourth material n₄ and the refractive index of the second organic material n₂ is 0.05 or more.

In an exemplary arrangement of the organic EL devices of the fourth, fifth, and sixth exemplary embodiments, the difference n₄−n₂ between the refractive index of the fourth material n₄ and the refractive index of the second organic material n₂ is 0.07 or more.

In an exemplary arrangement of the organic EL devices of the fourth, fifth, and sixth exemplary embodiments, the refractive index of the fourth material n₄ is 2.0 or more. By adding the fourth material having a higher refractive index to the second anode-side organic layer, the refractive index difference between the second anode-side organic layer and the third anode-side organic layer is more increased to further improve the light extraction efficiency of the organic EL device.

In an exemplary arrangement of the organic EL devices of the fourth, fifth, and sixth exemplary embodiments, a content of the fourth material in the second anode-side organic layer is 50 mass % or less.

In an exemplary arrangement of the organic EL devices of the fourth, fifth, and sixth exemplary embodiments, the content of the fourth material in the second anode-side organic layer is 40 mass % or less.

In an exemplary arrangement of the organic EL devices of the fourth, fifth, and sixth exemplary embodiments, the content of the fourth material in the second anode-side organic layer is 30 mass % or less.

The hole transportability can be ensured by setting the content of the fourth material in the second anode-side organic layer at 50 mass % or less and increasing the content of the second organic material.

In an exemplary arrangement of the organic EL devices of the fourth, fifth, and sixth exemplary embodiments, the content of the fourth material in the second anode-side organic layer is 1 mass % or more, 5 mass % or more, or 10 mass % or more.

In an exemplary arrangement of the organic EL devices of the fourth, fifth, and sixth exemplary embodiments, the third anode-side organic layer contains the third organic material and the fifth material.

In an exemplary arrangement of the organic EL devices of the fourth, fifth, and sixth exemplary embodiments, the refractive index NM₃₅ is smaller than the refractive index NM₃₀ as shown in the numerical formula (Numerical Formula NX3). By containing the fifth material as the additive material in the third anode-side organic layer, the refractive index of the third anode-side organic layer is decreased. As a result, the difference NM₂−NM₃ in refractive index between the second anode-side organic layer and the third anode-side organic layer is easily increased.

In an exemplary arrangement of the organic EL devices of the fourth, fifth, and sixth exemplary embodiments, a content of the fifth material in the third anode-side organic layer is 50 mass % or less.

In an exemplary arrangement of the organic EL devices of the fourth, fifth, and sixth exemplary embodiments, the content of the fifth material in the third anode-side organic layer is 40 mass % or less.

In an exemplary arrangement of the organic EL devices of the fourth, fifth, and sixth exemplary embodiments, a content of the fifth material in the third anode-side organic layer is 30 mass % or less.

In an exemplary arrangement of the organic EL devices of the fourth, fifth, and sixth exemplary embodiments, the content of the fifth material in the third anode-side organic layer is 1 mass % or more, 5 mass % or more, or 10 mass % or more.

In an exemplary arrangement of the organic EL devices of the fourth, fifth, and sixth exemplary embodiments, the second anode-side organic layer contains the second organic material and the fourth material, and the third anode-side organic layer contains the third organic material and the fifth material. In this arrangement, since the second anode-side organic layer and the third anode-side organic layer satisfy both of the numerical formulae (Numerical Formula NX2 and Numerical Formula NX3), the difference NM₂−NM₃ in refractive index between the second anode-side organic layer and the third anode-side organic layer is more easily increased.

In an exemplary arrangement of the organic EL devices of the fourth, fifth, and sixth exemplary embodiments, the first anode-side organic layer and the third anode-side organic layer satisfy a numerical formula (Numerical Formula NX5) below.

NM₁>NM₃ (Numerical Formula NX5)

In the numerical formula (Numerical Formula NX5), NM₁ is a refractive index of the constituent material contained in the first anode-side organic layer and NM₃ is a refractive index of the constituent material contained in the third anode-side organic layer.

In an exemplary arrangement of the organic EL devices of the fourth, fifth, and sixth exemplary embodiments, a total film thickness of the first hole transporting zone is in a range from 45 nm to 150 nm.

In an exemplary arrangement of the organic EL devices of the fourth, fifth, and sixth exemplary embodiments, the total film thickness of the first hole transporting zone is in a range from 25 nm to 85 nm.

In an exemplary arrangement of the organic EL devices of the fourth, fifth, and sixth exemplary embodiments, the total film thickness of the first hole transporting zone is in a range from 90 nm to 130 nm.

In an exemplary arrangement of the organic EL devices of the fourth, fifth, and sixth exemplary embodiments, a film thickness of the third anode-side organic layer is 20 nm or more. The light extraction efficiency is easily improved when the film thickness of the third anode-side organic layer satisfying the relationship of the numerical formula (Numerical Formula NX1) is 20 nm or more.

In an exemplary arrangement of the organic EL devices of the fourth, fifth, and sixth exemplary embodiments, the film thickness of the third anode-side organic layer is in a range from 20 nm to 70 nm.

In an exemplary arrangement of the organic EL devices of the fourth, fifth, and sixth exemplary embodiments, the film thickness of the second anode-side organic layer is 20 nm or more.

In an exemplary arrangement of the organic EL devices of the fourth, fifth, and sixth exemplary embodiments, the film thickness of the second anode-side organic layer is 70 nm or less.

In an exemplary arrangement of the organic EL devices of the fourth, fifth, and sixth exemplary embodiments, the film thickness of the first anode-side organic layer is 3 nm or more.

In an exemplary arrangement of the organic EL devices of the fourth, fifth, and sixth exemplary embodiments, the film thickness of the first anode-side organic layer is 20 nm or less.

In an exemplary arrangement of the organic EL devices of the fourth, fifth, and sixth exemplary embodiments, the first anode-side organic layer contains the acceptor material.

In an exemplary arrangement of the organic EL devices of the fourth, fifth, and sixth exemplary embodiments, the first anode-side organic layer may consist of a single type of material.

In an exemplary arrangement of the organic EL devices of the fourth, fifth, and sixth exemplary embodiments, the first anode-side organic layer may consist of a single type of acceptor material.

In an exemplary arrangement of the organic EL devices of the fourth, fifth, and sixth exemplary embodiments, the first anode-side organic layer is a hole injecting layer.

In an exemplary arrangement of the organic EL devices of the fourth, fifth, and sixth exemplary embodiments, the first anode-side organic layer contains the first organic material and the acceptor material. The first organic material and the acceptor material are mutually different compounds.

In an exemplary arrangement of the organic EL devices of the fourth, fifth, and sixth exemplary embodiments, a content of the acceptor material in the first anode-side organic layer is less than 50 mass %.

In an exemplary arrangement of the organic EL devices of the fourth, fifth, and sixth exemplary embodiments, the first anode-side organic layer contains the sixth material as the additive material. The sixth material is an organic material or a metal atom-containing material.

In an exemplary arrangement of the organic EL devices of the fourth, fifth, and sixth exemplary embodiments, in a case where the first anode-side organic layer contains the sixth material as the additive material, the first anode-side organic layer satisfies a numerical formula (Numerical Formula NX6) below.

NM₁₆>NM₁₀ (Numerical Formula NX6)

In the numerical formula (Numerical Formula NX6):
NM₁₀ represents a refractive index of a constituent material contained in the first anode-side organic layer not containing the sixth material; and
NM₁₆ represents a refractive index of the constituent material contained in the first anode-side organic layer containing the sixth material.

In an exemplary arrangement of the organic EL devices of the fourth, fifth, and sixth exemplary embodiments, the first hole transporting zone includes a fourth anode-side organic layer between the third anode-side organic layer and the first emitting region. In this arrangement, the first anode-side organic layer, the second anode-side organic layer, the third anode-side organic layer, and the fourth anode-side organic layer are disposed in this order from a side close to the anode. In an exemplary arrangement of the organic EL devices of the fourth, fifth, and sixth exemplary embodiments, the fourth anode-side organic layer contains the seventh organic material.

In an exemplary arrangement of the organic EL devices of the fourth, fifth, and sixth exemplary embodiments, the fourth-side organic layer is in direct contact with the third anode-side organic layer.

In an exemplary arrangement of the organic EL devices of the fourth, fifth, and sixth exemplary embodiments, the fourth anode-side organic layer is in direct contact with the first emitting region.

In an exemplary arrangement of the organic EL devices of the fourth, fifth, and sixth exemplary embodiments, the fourth anode-side organic layer is an electron blocking layer.

Preferably, the electron blocking layer permits transport of holes and blocks electrons from reaching a layer provided closer to the anode (e.g., the hole transporting layer) beyond the electron blocking layer.

In an exemplary arrangement of the organic EL devices of the fourth, fifth, and sixth exemplary embodiments, the fourth anode-side organic layer may block excitons generated in the emitting layer from transferring to the layers provided closer to the anode (e.g., the first anode-side organic layer, the second anode-side organic layer, and the third anode-side organic layer) beyond the fourth anode-side organic layer so as to avoid excited energy from leaking out from the emitting layer to neighboring layers.

In an exemplary arrangement of the organic EL devices of the fourth, fifth, and sixth exemplary embodiments, the film thickness of the fourth anode-side organic layer is 15 nm or less.

In an exemplary arrangement of the organic EL devices of the fourth, fifth, and sixth exemplary embodiments, the film thickness of the fourth anode-side organic layer is 10 nm or less.

In an exemplary arrangement of the organic EL devices of the fourth, fifth, and sixth exemplary embodiments, the film thickness of the fourth anode-side organic layer is 3 nm or more.

In an exemplary arrangement of the organic EL devices of the fourth, fifth, and sixth exemplary embodiments, the film thickness of the fourth anode-side organic layer is smaller than the film thickness of the third anode-side organic layer. The organic EL devices of the fourth, fifth, and sixth exemplary embodiments are considered to prolong the lifetime by including the fourth anode-side organic layer (preferably the electron blocking layer) having the smaller film thickness than the third anode-side organic layer.

In an exemplary arrangement of the organic EL devices of the fourth, fifth, and sixth exemplary embodiments, the first hole transporting zone includes a first mixture layer between the second anode-side organic layer and the third anode-side organic layer. In this arrangement, the first anode-side organic layer, the second anode-side organic layer, the first mixture layer, and the third anode-side organic layer are disposed in this order from a side close to the anode.

In an exemplary arrangement of the organic EL devices of the fourth, fifth, and sixth exemplary embodiments, the first mixture layer contains the second organic material, the third organic material, and at least one of the fourth material or the fifth material as the additive material.

In an exemplary arrangement of the organic EL devices of the fourth, fifth, and sixth exemplary embodiments, a film thickness of the first mixture layer may be 10 nm or more. In an exemplary arrangement of the organic EL devices of the fourth, fifth, and sixth exemplary embodiments, the film thickness of the first mixture layer is 50 nm or less. In an exemplary arrangement of the organic EL devices of the fourth, fifth, and sixth exemplary embodiments, the film thickness of the first mixture layer is smaller than the film thickness of the second anode-side organic layer and smaller than the film thickness of the third anode-side organic layer.

In an exemplary arrangement of the organic EL devices of the fourth, fifth, and sixth exemplary embodiments, the first mixture layer may be formed between the second anode-side organic layer and the third anode-side organic layer when the second anode-side organic layer and the third anode-side organic layer are successively formed in the same deposition chamber.

In an exemplary arrangement of the organic EL devices of the fourth, fifth, and sixth exemplary embodiments, the first hole transporting zone includes a second mixture layer between the first anode-side organic layer and the second anode-side organic layer. In this arrangement, the first anode-side organic layer, the second mixture layer, the second anode-side organic layer, and the third anode-side organic layer are disposed in this order from a side close to the anode.

In an exemplary arrangement of the organic EL devices of the fourth, fifth, and sixth exemplary embodiments, the second mixture layer contains the acceptor material, the second organic material, and the fourth material as the additive material.

In an exemplary arrangement of the organic EL devices of the fourth, fifth, and sixth exemplary embodiments, the second mixture layer contains the acceptor material, the first organic material, the second organic material, and the fourth material as the additive material.

In an exemplary arrangement of the organic EL devices of the fourth, fifth, and sixth exemplary embodiments, a film thickness of the second mixture layer may be 10 nm or more. In an exemplary arrangement of the organic EL devices of the fourth, fifth, and sixth exemplary embodiments, the film thickness of the second mixture layer is 50 nm or less. In an exemplary arrangement of the organic EL devices of the fourth, fifth, and sixth exemplary embodiments, the film thickness of the second mixture layer is smaller than the film thickness of the first anode-side organic layer and smaller than the film thickness of the second anode-side organic layer.

In an exemplary arrangement of the organic EL devices of the fourth, fifth, and sixth exemplary embodiments, the second mixture layer may be formed between the first anode-side organic layer and the second anode-side organic layer when first anode-side organic layer and the second anode-side organic layer are successively formed in the same deposition chamber.

In an exemplary arrangement of the organic EL devices of the fourth, fifth, and sixth exemplary embodiments, in a case where the first hole transporting zone includes the first mixture layer and the second mixture layer, the first anode-side organic layer, the second mixture layer, the second anode-side organic layer, the first mixture layer, and the third anode-side organic layer are disposed in this order from a side close to the anode.

### Hole Transporting Zone Material

The organic materials contained in the first hole transporting zone (i.e., the first organic material, the second organic material, the third organic material, and the seventh organic material) may be occasionally referred to as a hole transporting zone material.

The first organic material is one material forming the constituent material contained in the first anode-side organic layer. The first anode-side organic layer may consist of the first organic material or further contain another material.

The second organic material is one material forming the constituent material contained in the second anode-side organic layer. The second anode-side organic layer may consist of the second organic material or contain another material.

The third organic material is one material forming the constituent material contained in the third anode-side organic layer. The third anode-side organic layer may consist of the third organic material or further contain another material.

The seventh organic material is one material forming the constituent material contained in the fourth anode-side organic layer. The fourth anode-side organic layer may consist of the seventh organic material or contain another material.

In an exemplary arrangement of the organic EL devices of the fourth, fifth, and sixth exemplary embodiments, the first organic material, the second organic material, the third organic material, and the seventh organic material are each independently a monoamine compound having one substituted or unsubstituted amino group in a molecule, or a diamine compound having two substituted or unsubstituted amino groups in a molecule.

### First, Second, Third, and Seventh Organic Materials

In an exemplary arrangement of the organic EL devices of the fourth, fifth, and sixth exemplary embodiments, the first organic material, the second organic material, the third organic material, and the seventh organic material are each independently at least one compound selected from the group consisting of a compound represented by the formula (C1) below and a compound represented by the formula (C3) below.

In an exemplary arrangement of the organic EL devices of the fourth, fifth, and sixth exemplary embodiments, although the first anode-side organic layer, the second anode-side organic layer, the third anode-side organic layer and the fourth anode-side organic layer may each contain a compound represented by the formula (C1) below, the compound contained in the second anode-side organic layer and the compound contained in the third anode-side organic layer are mutually different in molecular structure, and the compound contained in the third anode-side organic layer and the compound contained in the fourth anode-side organic layer are mutually different in molecular structure.

In an exemplary arrangement of the organic EL devices of the fourth, fifth, and sixth exemplary embodiments, although the first anode-side organic layer, the second anode-side organic layer, the third anode-side organic layer and the fourth anode-side organic layer may each contain a compound represented by the formula (C3) below, the compound contained in the second anode-side organic layer and the compound contained in the third anode-side organic layer are mutually different in molecular structure, and the compound contained in the third anode-side organic layer and the compound contained in the fourth anode-side organic layer are mutually different in molecular structure.

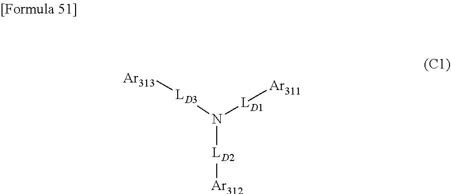

In the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms.

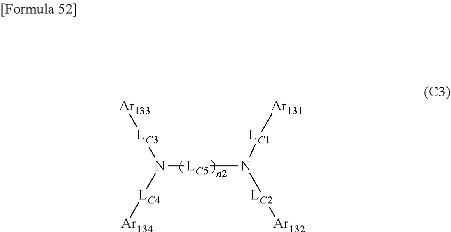

In the formula (C3):
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n₂ is 1, 2, 3, or 4;
when n₂ is 1, L_{C5} is a substituted or unsubstituted arylene group having 6 to 50 ring carbon atoms, or a substituted or unsubstituted divalent heterocyclic group having 5 to 50 ring atoms;
when n₂ is 2, 3, or 4, a plurality of L_{C5} are mutually the same or different;
when n₂ is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

In an exemplary arrangement of the organic EL devices of the fourth, fifth, and sixth exemplary embodiments, a substituent for the “substituted or unsubstituted” group in the compound represented by the formula (C1) and the compound represented by the formula (C3) is not a group represented by —N(R_{C6})(R_{C7}), in which R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of Rce are mutually the same or different, and a plurality of R_{C7} are mutually the same or different.

In an exemplary arrangement of the organic EL devices of the fourth, fifth, and sixth exemplary embodiments, a first amino group represented by the formula (C3-1) below and a second amino group represented by the formula (C3-2) below in the compound represented by the formula (C3) are an identical group.

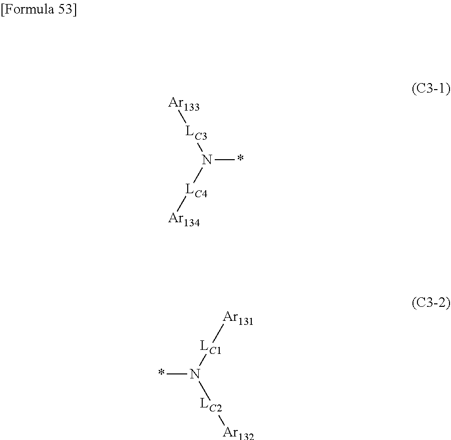

In the formulae (C3-1) and (C3-2), * each represent a bonding position to L_{C5}.

In an exemplary arrangement of the organic EL devices of the fourth, fifth, and sixth exemplary embodiments, the first amino group represented by the formula (C3-1) and the second amino group represented by the formula (C3-2) may be mutually different groups.

In an exemplary arrangement of the organic EL devices of the fourth, fifth, and sixth exemplary embodiments, the compound represented by the formula (C1) is a compound represented by a formula (C11) below.

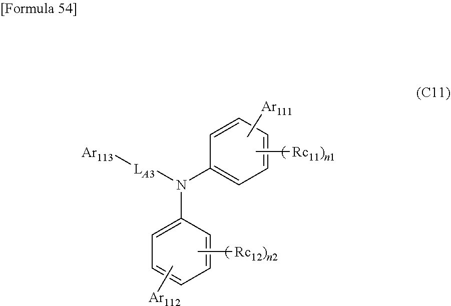

In the formula (C11):
Ar₁₁₁, Ar₁₁₂, Ar₁₁₃, and L_{A3} respectively represent the same as Ar₃₁₁, Ar₃₁₂, Ar₃₁₃, and L_{D3} in the formula (C1);
n₁ and n₂ are 4;
a plurality of R_{C11} are mutually the same or different;
at least one combination of adjacent two or more of a plurality of R_{C11} are mutually bonded to form a substituted or unsubstituted monocyclic ring, mutually bonded to form a substituted or unsubstituted fused ring, or not mutually bonded;
a plurality of R_{C12} are mutually the same or different;
at least one combination of adjacent two or more of a plurality of R_{C12} are mutually bonded to form a substituted or unsubstituted monocyclic ring, mutually bonded to form a substituted or unsubstituted fused ring, or not mutually bonded; and
R_{C11} and R_{C12} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms.

In the compound represented by the formula (C1), at least one of Ar₃₁₁, Ar₃₁₂, or Ar₃₁₃ is preferably a group selected from the group consisting of groups represented by the formulae (21a), (21b), (21c), (21d), and (21e) below.

In the compound represented by the formula (C11), at least one of Ar₁₁₁, Ar₁₁₂, or Ar₁₁₃ is preferably a group selected from the group consisting of groups represented by the formulae (21a), (21b), (21c), (21d), and (21e) below.

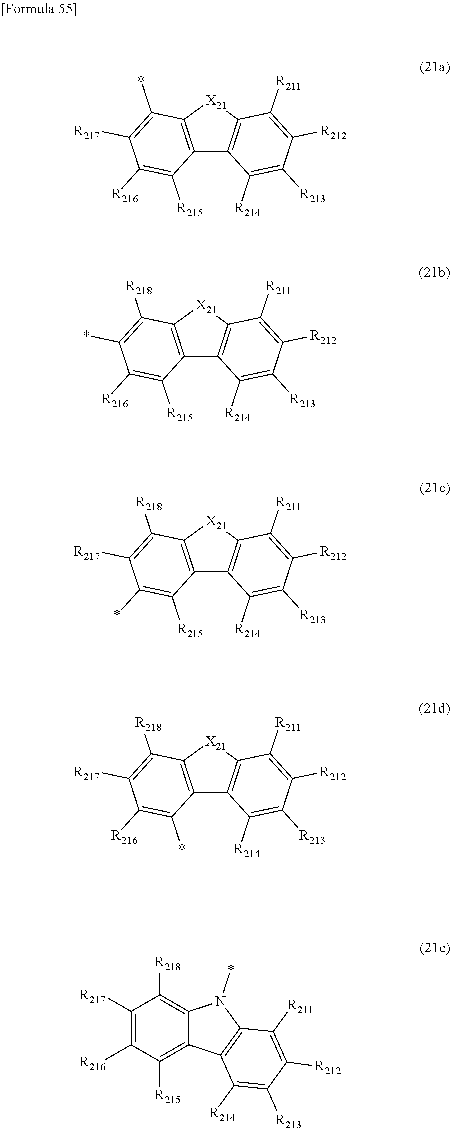

In the formulae (21a), (21b), (21c), (21d), and (21e):
X₂₁ is NR₂₁, CR₂₂R₂₃, an oxygen atom, or a sulfur atom;
when a plurality of X₂₁ are present, the plurality of X₂₁ are mutually the same or different;
when X₂₁ is CR₂₂R₂₃, a combination of R₂₂ and R₂₃ are mutually bonded to form a substituted or unsubstituted monocyclic ring, mutually bonded to form a substituted or unsubstituted fused ring, or not mutually bonded;
R₂₁, and R₂₂ and R₂₃ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded;
R₂₁₁ to R₂₁₈ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms; and
* in the formulae (21a), (21b), (21c), (21d), and (21e) are each independently a bonding position to L_{D1}, L_{D2}, L_{D3}, L_{A1}, L_{A2}, and L_{A3}.

Ar₁₁₁, Ar₁₁₂, and Ar₁₁₃ not being a group selected from the group consisting of groups represented by the formulae (21a), (21b), (21c), (21d), and (21e) are each independently preferably a substituted or unsubstituted aryl group having 6 to 30 ring carbon atoms, more preferably a substituted or unsubstituted phenyl group or a substituted or unsubstituted biphenyl group.

In the compound represented by the formula (C1), it is also preferable that two of Ar₃₁₁, Ar₃₁₂, and Ar₃₁₃ are each a group selected from the group consisting of groups represented by the formulae (21a), (21b), (21c), (21d), and (21e) and the remaining one of Ar₃₁₁, Ar₃₁₂, and Ar₃₁₃ is a substituted or unsubstituted aryl group having 6 to 30 ring carbon atoms.

In the compound represented by the formula (C11), it is also preferable that two of Ar₁₁₁, Ar₁₁₂, and Ar₁₁₃ are each a group selected from the group consisting of groups represented by the formulae (21a), (21b), (21c), (21d), and (21e) and the remaining one of Ar₁₁₁, Ar₁₁₂, and Ar₁₁₃ is a substituted or unsubstituted aryl group having 6 to 30 ring carbon atoms.

In the compound represented by the formula (C1), it is also preferable that one of Ar₃₁₁, Ar₃₁₂, and Ar₃₁₃ is a group selected from the group consisting of groups represented by the formulae (21a), (21b), (21c), (21d), and (21e) and the remaining two of Ar₃₁₁, Ar₃₁₂, and Ar₃₁₃ are each a substituted or unsubstituted aryl group having 6 to 30 ring carbon atoms.

In the compound represented by the formula (C11), it is also preferable that one of Ar₁₁₁, Ar₁₁₂, and Ar₁₁₃ is a group selected from the group consisting of groups represented by the formulae (21a), (21b), (21c), (21d), and (21e) and the remaining two of Ar₁₁₁, Ar₁₁₂, and Ar₁₁₃ are each a substituted or unsubstituted aryl group having 6 to 30 ring carbon atoms.

In an exemplary arrangement of the organic EL devices of the fourth, fifth, and sixth exemplary embodiments, the compound represented by the formula (C1) is a monoamine compound.

### Second Organic Material

In an exemplary arrangement of the organic EL devices of the fourth, fifth, and sixth exemplary embodiments, the second anode-side organic layer contains at least one compound selected from the group consisting of a compound represented by the formula (C1) and a compound represented by the formula (C3).

In an exemplary arrangement of the organic EL devices of the fourth, fifth, and sixth exemplary embodiments, the second organic material is a compound represented by the formula (C1) or a compound represented by the formula (C3).

In an exemplary arrangement of the organic EL devices of the fourth, fifth, and sixth exemplary embodiments, the second anode-side organic layer contains at least one compound selected from the group consisting of a compound represented by a formula (cHT2-1), a compound represented by a formula (cHT2-2), and a compound represented by a formula (cHT2-3) below.

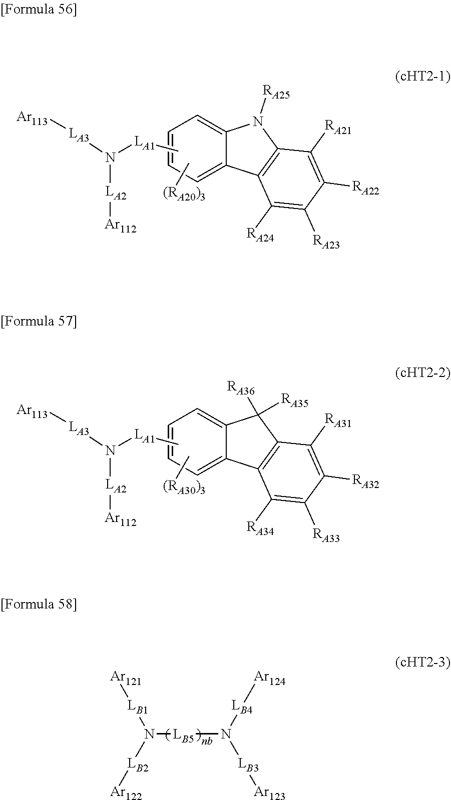

In the formulae (cHT2-1), (cHT2-2), and (cHT2-3):
Ar₁₁₂, Ar₁₁₃, Ar₁₂₁, Ar₁₂₂, Ar₁₂₃, and Ar₁₂₄ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{A1}, L_{A2}, L_{A3}, L_{B1}, L_{B2}, L_{B3}, and L_{B4} are each independently a single bond, a substituted or unsubstituted arylene group having 6 to 50 ring carbon atoms, or a substituted or unsubstituted divalent heterocyclic group having 5 to 50 ring atoms;
nb is 1, 2, 3 or 4;
when nb is 1, L_{B5} is a substituted or unsubstituted arylene group having 6 to 50 ring carbon atoms, or a substituted or unsubstituted divalent heterocyclic group having 5 to 50 ring atoms;
when nb is 2, 3, or 4, a plurality of L_{B5} are mutually the same or different;
when nb is 2, 3, or 4, a plurality of L_{B5} are mutually bonded to form a substituted or unsubstituted monocyclic ring, mutually bonded to form a substituted or unsubstituted fused ring, or not mutually bonded;
L_{B5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
a combination of R_{A35} and R_{A36} are mutually bonded to form a substituted or unsubstituted monocyclic ring, mutually bonded to form a substituted or unsubstituted fused ring, or not mutually bonded;
R_{A25}, and R_{A35} and R_{A36} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R_{A20} to R_{A24} are mutually bonded to form a substituted or unsubstituted monocyclic ring, mutually bonded to form a substituted or unsubstituted fused ring, or not mutually bonded;
at least one combination of adjacent two or more of R_{A30} to R_{A34} are mutually bonded to form a substituted or unsubstituted monocyclic ring, mutually bonded to form a substituted or unsubstituted fused ring, or not mutually bonded;
R_{A20} to R_{A24} and R_{A30} to R_{A34} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
a plurality of R_{A20} are mutually the same or different;
a plurality of R_{A30} are mutually the same or different;
R₉₀₁ to R₉₀₄ in the compounds represented by the formulae (cHT2-1), (cHT2-2), and (cHT2-3) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

In an exemplary arrangement of the organic EL devices of the fourth, fifth, and sixth exemplary embodiments, a first amino group represented by a formula (c21) below and a second amino group represented by a formula (c22) below in the compound represented by the formula (cHT2-3) are an identical group or different groups.

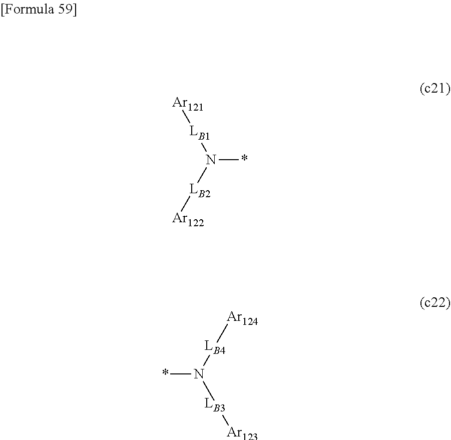

In the formulae (c21) and (c22), * each represent a bonding position to L_{B5}.

In an exemplary arrangement of the organic EL devices of the fourth, fifth, and sixth exemplary embodiments, a substituent for a “substituted or unsubstituted” group in the compounds represented by the formulae (cHT2-1), (cHT2-2), and (cHT2-3) is not a group represented by —N(R_{C6})(R_{C7}).

In an exemplary arrangement of the organic EL devices of the fourth, fifth, and sixth exemplary embodiments, R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of Rce are mutually the same or different, and a plurality of R_{C7} are mutually the same or different.

In an exemplary arrangement of the organic EL devices of the fourth, fifth, and sixth exemplary embodiments, when a substituent for a “substituted or unsubstituted” group is not a group represented by —N(R_{C6})(R_{C7}), the compounds represented by the formulae (cHT2-1) and (cHT2-2) are each a monoamine compound.

In an exemplary arrangement of the organic EL devices of the fourth, fifth, and sixth exemplary embodiments, when a substituent for a “substituted or unsubstituted” group is not a group represented by —N(R_{C6})(R_{C7}), the compound represented by the formulae (cHT2-3) is a diamine compound.

In an exemplary arrangement of the organic EL devices of the fourth, fifth, and sixth exemplary embodiments, the second organic material is a compound represented by the formula (cHT2-1), a compound represented by the formula (cHT2-2), or a compound represented by the formula (cHT2-3).

In an exemplary arrangement of the organic EL devices of the fourth, fifth, and sixth exemplary embodiments, the second organic material has at least one group selected from the group consisting of a group represented by the formula (2-a), a group represented by the formula (2-b), a group represented by the formula (2-c), a group represented by the formula (2-d), a group represented by the formula (2-e), and a group represented by the formula (2-f) below.

In an exemplary arrangement of the organic EL devices of the fourth, fifth, and sixth exemplary embodiments, the second organic material is at least one compound selected from the group consisting of a compound represented by the formula (cHT2-1), a compound represented by the formula (cHT2-2), and a compound represented by the formula (cHT2-3), and at least one of Ar₁₁₂, Ar₁₁₃, Ar₁₂₁, Ar₁₂₂, Ar₁₂₃, or Ar₁₂₄ in the formulae (cHT2-1), (cHT2-2), and (cHT2-3) has at least one group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

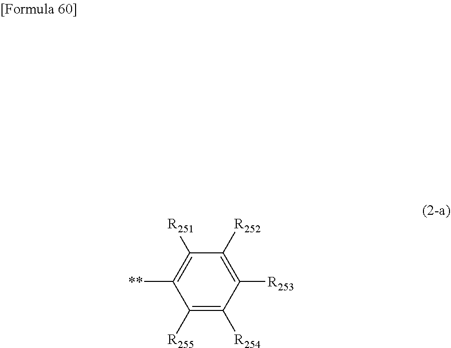

In the formula (2-a):
none of a combination(s) of adjacent two or more of R₂₅₁ to R₂₅₅ are bonded to each other;
R₂₅₁ to R₂₅₅ are each independently a hydrogen atom, or a substituted or unsubstituted alkyl group having 1 to 6 carbon atoms; and
** represents a bonding position.

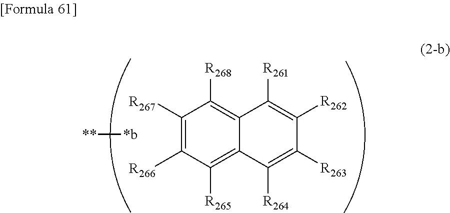

In the formula (2-b):
one of R₂₆₁ to R₂₆₈ is a single bond with *b;
none of a combination(s) of adjacent two or more of R₂₆₁ to R₂₆₈ not being the single bond with *b are bonded to each other;
R₂₆₁ to R₂₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position.

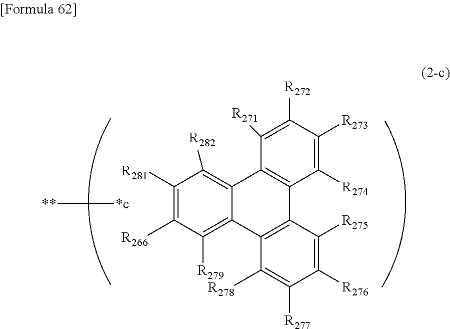

In the formula (2-c):
one of R₂₇₁ to R₂₈₂ is a single bond with *c;
none of a combination(s) of adjacent two or more of R₂₇₁ to R₂₈₂ not being the single bond with *c are bonded to each other;
R₂₇₁ to R₂₈₂ not being the single bond with *c are each independently a hydrogen atom, a substituted or unsubstituted alkyl group having 1 to 6 carbon atoms, or a substituted or unsubstituted aryl group having 6 to 12 ring carbon atoms; and
** represents a bonding position.

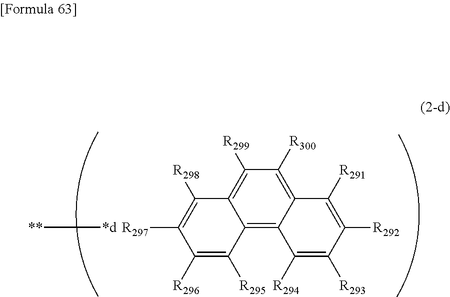

In the formula (2-d):
one of R₂₉₁ to R₃₀₀ is a single bond with *d;
none of a combination(s) of adjacent two or more of R₂₉₁ to R₃₀₀ not being the single bond with *d are bonded to each other;
R₂₉₁ to R₃₀₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position.

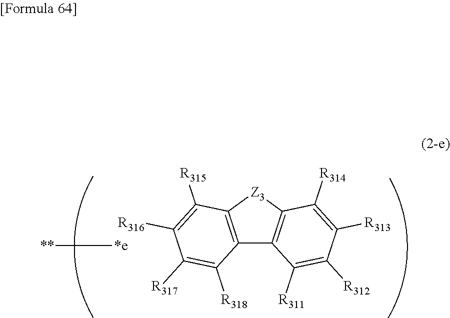

In the formula (2-e):
Z₃ is an oxygen atom, a sulfur atom, NR₃₁₉, or C(R₃₂₀)(R₃₂₁);
one of R₃₁₁ to R₃₂₁ is a single bond with *e, or one of carbon atoms of a substituted or unsubstituted benzene ring below formed by mutually bonding a combination of adjacent two or more of R₃₁₁ to R₃₁₈ is bonded to *e by a single bond;
a combination of adjacent two or more of R₃₁₁ to R₃₁₈ not being the single bond with *e are mutually bonded to form a substituted or unsubstituted benzene ring, or not mutually bonded;
R₃₁₁ to R₃₁₈ not being the single bond with *e and not forming the substituted or unsubstituted benzene ring are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

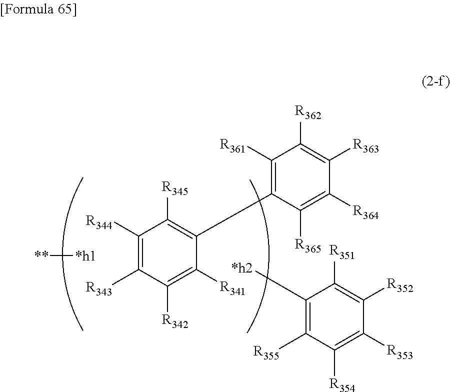

In the formula (2-f):
one of R₃₄₁ to R₃₄₅ is a single bond with *h1, and another one of R₃₄₁ to R₃₄₅ is a single bond with *h2;
none of a combination(s) of adjacent two or more of R₃₄₁ to R₃₄₅ not being the single bond with *h1 and not being the single bond with *h2 are bonded to each other;
at least one combination of adjacent two or more of R₃₅₁ to R₃₅₅ are mutually bonded to form a substituted or unsubstituted monocyclic ring, mutually bonded to form a substituted or unsubstituted fused ring, or not mutually bonded;
at least one combination of adjacent two or more of R₃₆₁ to R₃₆₅ are mutually bonded to form a substituted or unsubstituted monocyclic ring, mutually bonded to form a substituted or unsubstituted fused ring, or not mutually bonded;
R₃₄₁ to R₃₄₅ not being the single bond with *h1 and not being the single bond with *h2 as well as R₃₅₁ to R₃₅₅ and R₃₆₁ to R₃₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

In an exemplary arrangement of the organic EL devices of the fourth, fifth, and sixth exemplary embodiments, a group represented by the formula (2-a), a group represented by the formula (2-b), a group represented by the formula (2-c), a group represented by the formula (2-d), a group represented by the formula (2-e), and a group represented by the formula (2-f) are each independently bonded directly, through a phenylene group, or through a biphenylene group to a nitrogen atom of an amino group of the monoamine compound.

In an exemplary arrangement of the organic EL devices of the fourth, fifth, and sixth exemplary embodiments, the second organic material is a monoamine compound having one substituted or unsubstituted amino group in a molecule.

In an exemplary arrangement of the organic EL devices of the fourth, fifth, and sixth exemplary embodiments, the second organic material is a monoamine compound having one substituted or unsubstituted amino group in a molecule, and a group represented by the formula (2-a), a group represented by the formula (2-b), a group represented by the formula (2-c), a group represented by the formula (2-d), a group represented by the formula (2-e), and a group represented by the formula (2-f) are each independently bonded directly, through a phenylene group, or through a biphenylene group to a nitrogen atom of an amino group of the monoamine compound.

In an exemplary arrangement of the organic EL devices of the fourth, fifth, and sixth exemplary embodiments, the second organic material is a compound represented by the formula (cHT2-1) and at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL devices of the fourth, fifth, and sixth exemplary embodiments, the second organic material is a compound represented by the formula (cHT2-2) and at least one of Ar₁₁₂ or Ar₁₁₃ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL devices of the fourth, fifth, and sixth exemplary embodiments, the second organic material is a compound represented by the formula (cHT2-3) and at least one of Ar₁₂₁, Ar₁₂₂, Ar₁₂₃, or Ar₁₂₄ is a group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In the organic EL devices of the fourth, fifth, and sixth exemplary embodiments, when Z₃ in the formula (2-e) is NR₃₁₉, R₃₁₂ or R₃₁₇ is preferably a single bond with *e.

In an exemplary arrangement of the organic EL devices of the fourth, fifth, and sixth exemplary embodiments, the group represented by the formula (2-e) is a group represented by a formula (2-e7) below.

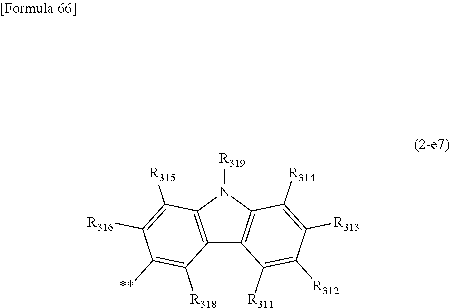

In the formula (2-e7), R₃₁₁ to R₃₁₆, R₃₁₈, and R₃₁₉ respectively represent the same as R₃₁₁ to R₃₁₆, R₃₁₈, and R₃₁₉ in the formula (2-e), and ** represents a bonding position.

In the organic EL devices of the fourth, fifth, and sixth exemplary embodiments, when Z₃ in the formula (2-e) is NR₃₁₉, R₃₁₅, R₃₁₆, or R₃₁₈ is also preferably a single bond with *e.

In an exemplary arrangement of the organic EL devices of the fourth, fifth, and sixth exemplary embodiments, the group represented by the formula (2-e) is a group represented by a formula (2-e4), (2-e5), or (2-e6) below.

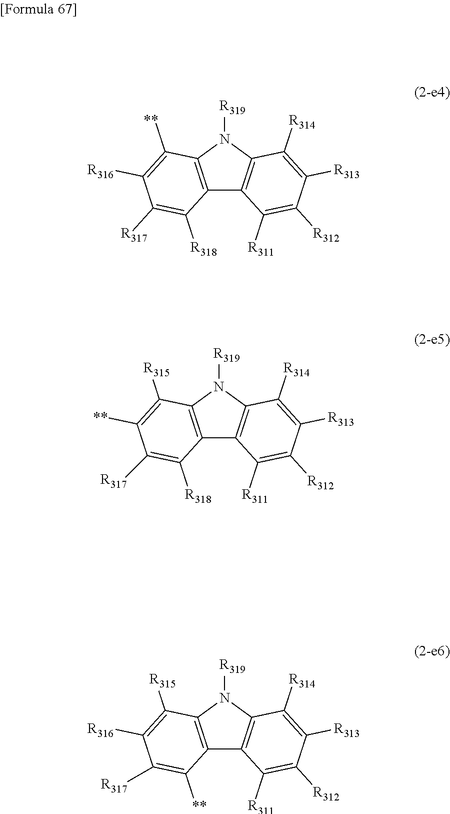

In the formulae (2-e4), (2-e5), and (2-66), R₃₁₁ to R₃₁₉ respectively represent 5 the same as R₃₁₁ to R₃₁₉ in the formula (2-e), and ** represents a bonding position.

In an exemplary arrangement of the organic EL devices of the fourth, fifth, and sixth exemplary embodiments, the group represented by the formula (2-e) is a group represented by a formula (2-e1), (2-e2), or (2-e3) below.

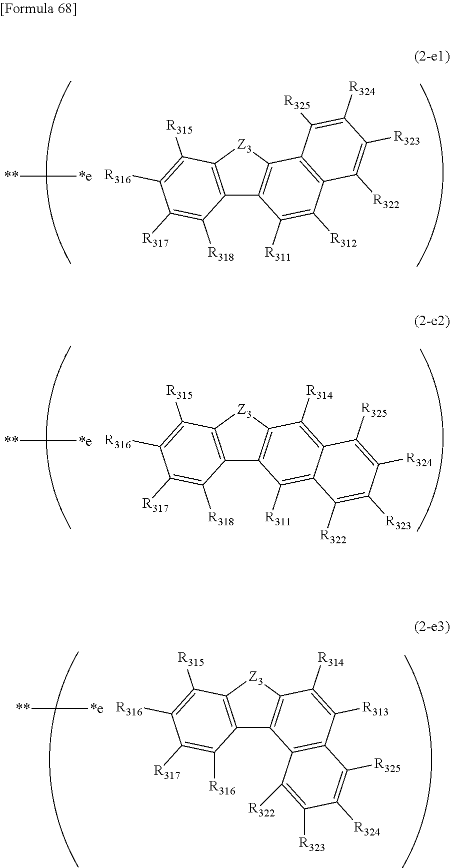

In the formulae (2-e1), (2-e2), and (2-e3):
Z₃ is an oxygen atom, a sulfur atom, NR₃₁₉, or C(R₃₂₀)(R₃₂₁);
one of R₃₁₁ to R₃₂₅ is a single bond with *e;
R₃₁₁ to R₃₁₈ and R₃₂₂ to R₃₂₅ not being the single bond with *e are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond with *e is a hydrogen atom, a substituted or unsubstituted alkyl group having 1 to 6 carbon atoms, or a substituted or unsubstituted aryl group having 6 to 12 ring carbon atoms;
a combination of R₃₂₀ and R₃₂₁ not being the single bond with *e are mutually bonded to form a substituted or unsubstituted monocyclic ring, mutually bonded to form a substituted or unsubstituted fused ring, or not mutually bonded;
R₃₂₀ and R₃₂₁ not being the single bond with *e, not forming the substituted or unsubstituted monocyclic ring, and not forming the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position.

In an exemplary arrangement of the organic EL devices of the fourth, fifth, and sixth exemplary embodiments, ** in the formulae (2-a), (2-b), (2-c), (2-d), (2-e), (2-f), (2-e1), (2-e2), and (2-e3) are each independently a bonding position to L_{A2}, L_{A3}, L_{B1}, L_{B2}, L_{B3}, or L_{B4} or a bonding position to a nitrogen atom of an amino group.

In an exemplary arrangement of the organic EL devices of the fourth, fifth, and sixth exemplary embodiments, the second organic material is a compound having no thiophene ring in a molecule.

In an exemplary arrangement of the organic EL devices of the fourth, fifth, and sixth exemplary embodiments, the refractive index of the second organic material n₂ is 1.8 or more. When the refractive index of the second organic material is increased, the refractive index of the second anode-side organic layer is increased and the refractive index difference between the second anode-side organic layer and the third anode-side organic layer is increased to improve the light extraction efficiency.

In an exemplary arrangement of the organic EL devices of the fourth, fifth, and sixth exemplary embodiments, an ionization potential of the second organic material is 5.7 eV or less.

Herein, the ionization potential of a compound is measured using a photoelectron spectrometer under atmosphere. Specifically, the ionization potential of a compound can be measured by the method described in Examples.

### Third Organic Material

In an exemplary arrangement of the organic EL devices of the fourth, fifth, and sixth exemplary embodiments, the third anode-side organic layer contains a compound represented by the formula (C1).

In an exemplary arrangement of the organic EL devices of the fourth, fifth, and sixth exemplary embodiments, the third organic layer is a compound represented by the formula (C1).

In an exemplary arrangement of the organic EL devices of the fourth, fifth, and sixth exemplary embodiments, the third anode-side organic layer contains at least one compound selected from the group consisting of a compound represented by the formula (cHT3-11), a compound represented by the formula (cHT3-2), a compound represented by the formula (cHT3-31), and a compound represented by the formula (cHT3-4) below.

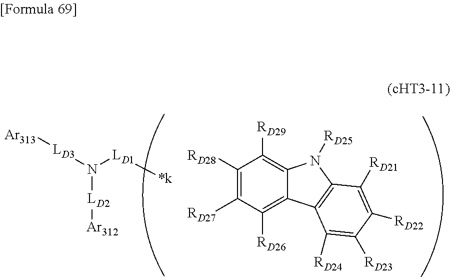

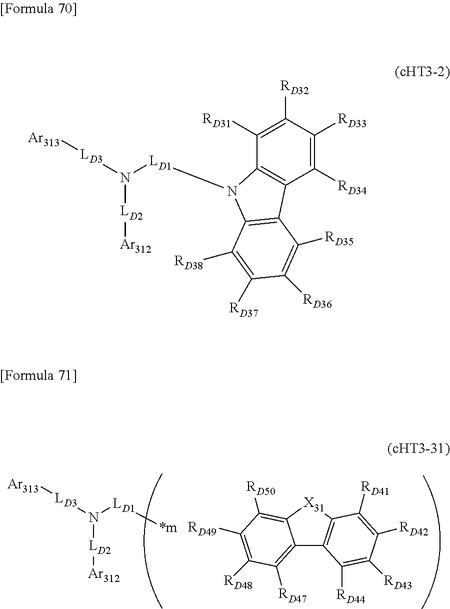

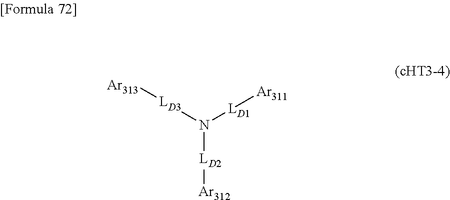

In the formulae (cHT3-11), (cHT3-2), (CHT3-31), and (cHT3-4):
Ar₃₁₁ is a group represented by one of a formula (1-a), a formula (1-b), a formula (1-c), and a formula (1-d) below;
Ar₃₁₂ and Ar₃₁₃ are each independently a substituted or unsubstituted aryl group having 6 to 50 ring carbon atoms, a substituted or unsubstituted heterocyclic group having 5 to 50 ring atoms, or a group represented by —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms;
one of R_{D26} to R_{D29} is a single bond with L_{D1} and *k represents a bonding position;
at least one combination of adjacent two or more of R_{D21} to R_{D24}, and R_{D26} to R_{D29} not being the single bond with L_{D1} are mutually bonded to form a substituted or unsubstituted monocyclic ring, mutually bonded to form a substituted or unsubstituted fused ring, or not mutually bonded;
at least one combination of adjacent two or more of R_{D31} to R_{D38} are mutually bonded to form a substituted or unsubstituted monocyclic ring, mutually bonded to form a substituted or unsubstituted fused ring, or not mutually bonded;
one of R_{D47} to R_{D50} is a single bond with L_{D1} and *m represents a bonding position;
at least one combination of adjacent two or more of R_{D41} to R_{D44}, and R_{D47} to R_{D50} not being the single bond with L_{D1} are mutually bonded to form a substituted or unsubstituted monocyclic ring, mutually bonded to form a substituted or unsubstituted fused ring, or not mutually bonded;
X₃₁ is an oxygen atom, a sulfur atom, or C(R_{D45}) (R_{D46});
a combination of R_{D45} and R_{D46} are mutually bonded to form a substituted or unsubstituted monocyclic ring, mutually bonded to form a substituted or unsubstituted fused ring, or not mutually bonded;
R_{D25}, and R_{D21} to R_{D24}, R_{D26} to R_{D29}, R_{D31} to R_{D38}, and R_{D41} to R_{D50} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
R₉₀₁ to R₉₀₄ in the compounds represented by the formulae (cHT3-11), (cHT3-2), (cHT3-31), and (cHT3-4) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

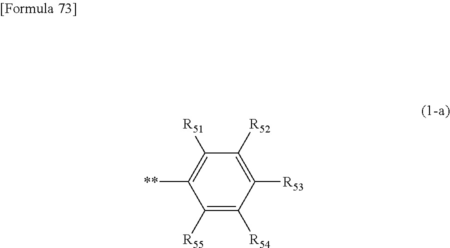

In the formula (1-a):
none of a combination(s) of adjacent two or more of R₅₁ to R₅₅ are bonded to each other;
R₅₁ to R₅₅ are each independently a hydrogen atom, or a substituted or unsubstituted alkyl group having 1 to 6 carbon atoms; and
** represents a bonding position to L_{D1}.

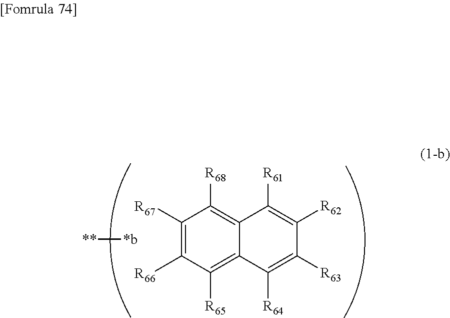

In the formula (1-b):
one of R₆₁ to Res is a single bond with *b;
none of a combination(s) of adjacent two or more of R₆₁ to R₆₈ not being the single bond with *b are bonded to each other;
R₆₁ to R₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

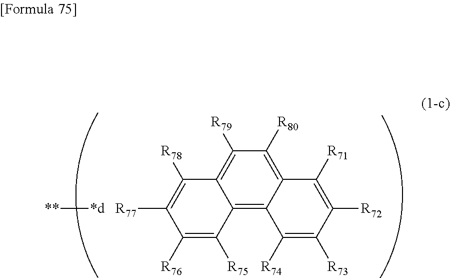

In the formula (1-c):
one of R₇₁ to Rao is a single bond with *d;
none of a combination(s) of adjacent two or more of R₇₁ to Rao not being the single bond with *d are bonded to each other;
R₇₁ to Rao not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

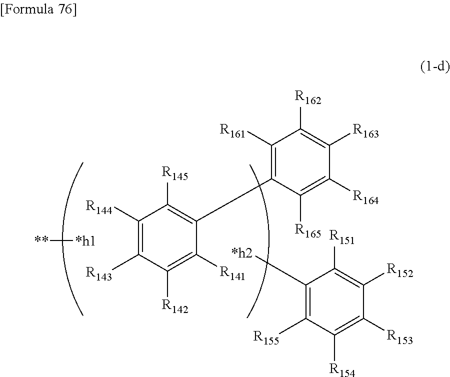

In the formula (1-d):
one of R₁₄₁ to R₁₄₅ is a single bond with *h1, and another one of R₁₄₁ to R₁₄₅ is a single bond with *h2;
none of a combination(s) of adjacent two or more of R₁₄₁ to R₁₄₅ not being the single bond with *h1 and not being the single bond with *h2 are bonded to each other;
at least one combination of adjacent two or more of R₁₅₁ to R₁₅₅ are mutually bonded to form a substituted or unsubstituted monocyclic ring, mutually bonded to form a substituted or unsubstituted fused ring, or not mutually bonded;
at least one combination of adjacent two or more of R₁₆₁ to R₁₆₅ are mutually bonded to form a substituted or unsubstituted monocyclic ring, mutually bonded to form a substituted or unsubstituted fused ring, or not mutually bonded;
R₁₄₁ to R₁₄₅ not being the single bond with *h1 and not being the single bond with *h2 as well as R₁₅₁ to R₁₅₅ and R₁₆₁ to R₁₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

In an exemplary arrangement of the organic EL devices of the fourth, fifth, and sixth exemplary embodiments, a substituent for the “substituted or unsubstituted” group in the compounds represented by the formulae (cHT3-11), (cHT3-2), (cHT3-31), and (cHT3-4) is not a group represented by —N(R_{C6})(R_{C7}), in which R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, a plurality of Rce are mutually the same or different, and a plurality of R_{C7} are mutually the same or different.

In an exemplary arrangement of the organic EL devices of the fourth, fifth, and sixth exemplary embodiments, when a substituent for a “substituted or unsubstituted” group is not a group represented by —N(R_{C6})(R_{C7}), the compounds represented by the formulae (cHT3-11), (cHT3-2), (CHT3-31), and (cHT3-4) are each a monoamine compound.

In an exemplary arrangement of the organic EL devices of the fourth, fifth, and sixth exemplary embodiments, the third organic material is a compound represented by the formula (cHT3-11), a compound represented by the formula (CHT3-2), a compound represented by the formula (cHT3-31), or a compound represented by the formula (cHT3-4).

In an exemplary arrangement of the organic EL devices of the fourth, fifth, and sixth exemplary embodiments, the third organic material is a monoamine compound having one substituted or unsubstituted amino group in a molecule.

In an exemplary arrangement of the organic EL devices of the fourth, fifth, and sixth exemplary embodiments, the third organic material is a compound having no thiophene ring in a molecule.

In an exemplary arrangement of the organic EL device of the fourth, fifth, and sixth exemplary embodiments, the second anode-side organic layer contains at least one compound selected from the group consisting of a compound represented by the formula (cHT2-1), a compound represented by the formula (cHT2-2), and a compound represented by the formula (cHT2-3), and the third anode-side organic layer contains at least one compound selected from the group consisting of a compound represented by the formula (cHT3-11), a compound represented by the formula (cHT3-2), a compound represented by the formula (cHT3-31), and a compound represented by the formula (cHT3-4).

In an exemplary arrangement of the organic EL devices of the fourth, fifth, and sixth exemplary embodiments, the refractive index of the third organic material is 1.85 or less.

In an exemplary arrangement of the organic EL devices of the fourth, fifth, and sixth exemplary embodiments, the refractive index of the third organic material is 1.80 or less.

### First Organic Material

In an exemplary arrangement of the organic EL devices of the fourth, fifth, and sixth exemplary embodiments, the first organic material contained in the first anode-side organic layer is at least one compound selected from the group consisting of a compound represented by the formula (C1), a compound represented by the formula (cHT2-1), a compound represented by the formula (cHT2-2), and a compound represented by the formula (cHT2-3).

In an exemplary arrangement of the organic EL devices of the fourth, fifth, and sixth exemplary embodiments, the first organic material contained in the first anode-side organic layer is a monoamine compound.

In an exemplary arrangement of the organic EL devices of the fourth, fifth, and sixth exemplary embodiments, the first organic material has at least one group selected from the group consisting of a group represented by the formula (2-a), a group represented by the formula (2-b), a group represented by the formula (2-c), a group represented by the formula (2-d), a group represented by the formula (2-e), and a group represented by the formula (2-f).

In an exemplary arrangement of the organic EL devices of the fourth, fifth, and sixth exemplary embodiments, the first organic material is a monoamine compound having one substituted or unsubstituted amino group in a molecule, and a group represented by the formula (2-a), a group represented by the formula (2-b), a group represented by the formula (2-c), a group represented by the formula (2-d), a group represented by the formula (2-e), and a group represented by the formula (2-f) are each independently bonded directly, through a phenylene group, or through a biphenylene group to a nitrogen atom of an amino group of the monoamine compound.

In an exemplary arrangement of the organic EL device of the fourth, fifth, and sixth exemplary embodiments, the first organic material is a compound selected from the group consisting of a compound represented by the formula (cHT2-1) and a compound represented by the formula (cHT2-2).

In an exemplary arrangement of the organic EL devices of the fourth, fifth, and sixth exemplary embodiments, the compound represented by the formula (cHT2-1) and the compound represented by the formula (cHT2-2), as the first organic material, are each a monoamine compound.

In an exemplary arrangement of the organic EL devices of the fourth, fifth, and sixth exemplary embodiments, the first organic material is a compound having no thiophene ring in a molecule.

### Seventh Organic Material

In an exemplary arrangement of the organic EL devices of the fourth, fifth, and sixth exemplary embodiments, the seventh organic material and the third organic material are mutually different compounds.

In an exemplary arrangement of the organic EL devices of the fourth, fifth, and sixth exemplary embodiments, the seventh organic material is a compound different from the first organic material, the second organic material, the third organic material, the fourth material, the fifth material, and the sixth material.

In an exemplary arrangement of the organic EL devices of the fourth, fifth, and sixth exemplary embodiments, the fourth anode-side organic layer contains a compound represented by the formula (C1) or a compound represented by the formula (C3).

In an exemplary arrangement of the organic EL devices of the fourth, fifth, and sixth exemplary embodiments, although the third anode-side organic layer and the fourth anode-side organic layer may each contain a compound represented by the formula (C1), the compound contained in the third anode-side organic layer and the compound contained in the fourth anode-side organic layer are mutually different in molecular structure.

In an exemplary arrangement of the organic EL devices of the fourth, fifth, and sixth exemplary embodiments, in a case where the fourth anode-side organic layer is an electron blocking layer, a compound contained in the electron blocking layer (the seventh organic material) is, for instance, a known compound used for the electron blocking layer and is at least one compound selected from the group consisting of an aromatic amine compound and a carbazole derivative. The compound contained in the electron blocking layer may be a monoamine compound. The compound contained in the electron blocking layer may be a compound having a substituted or unsubstituted carbazolyl group and one substituted or unsubstituted amino group in a molecule.

In an exemplary arrangement of the organic EL devices of the fourth, fifth, and sixth exemplary embodiments, the seventh organic material is a compound having no thiophene ring in a molecule.

In an exemplary arrangement of the organic EL devices of the fourth, fifth, and sixth exemplary embodiments, the first anode-side organic layer, the second anode-side organic layer, the third anode-side organic layer, and the fourth anode-side organic layer contain one or more compounds different from each other.

In an exemplary arrangement of the organic EL devices of the fourth, fifth, and sixth exemplary embodiments, the first anode-side organic layer, the second anode-side organic layer, the third anode-side organic layer, and the fourth anode-side organic layer each independently contain a monoamine compound having only one substituted or unsubstituted amino group in a molecule.

In an exemplary arrangement of the organic EL devices of the fourth, fifth, and sixth exemplary embodiments, the first anode-side organic layer, the second anode-side organic layer, the third anode-side organic layer, and the fourth anode-side organic layer contain no diamine compound.

In an exemplary arrangement of the organic EL devices of the fourth, fifth, and sixth exemplary embodiments, at least one of the first anode-side organic layer, the second anode-side organic layer, the third anode-side organic layer, or the fourth anode-side organic layer may also contain a diamine compound. The compound represented by the formula (C3) is preferably a diamine compound.

In the organic EL devices of the fourth, fifth, and sixth exemplary embodiments, R₉₀₁, R₉₀₂, R₉₀₃, and R₉₀₄ in a compound contained in each layer of the first hole transporting zone are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of R₉₀₃ are mutually the same or different; and
when a plurality of R₉₀₄ are present, the plurality of R₉₀₄ are mutually the same or different.

In the fourth, fifth, and sixth exemplary embodiments, the groups specified to be “substituted or unsubstituted” are each preferably an “unsubstituted” group.

In the organic EL devices according to the fourth, fifth, and sixth exemplary embodiments, the hole transporting zone material may be a compound having a substituted or unsubstituted 3-carbazolyl group in a molecule. Moreover, in the organic EL devices according to the fourth, fifth, and sixth exemplary embodiments, the hole transporting zone material may be a compound not having a substituted or unsubstituted 3-carbazolyl group in a molecule.

### Method of Producing Hole Transporting Zone Material

The hole transporting zone material according to the fourth, fifth, and sixth exemplary embodiments can be produced by a known method or through a known alternative reaction using a known material(s) tailored for the target compound in accordance with the known method.

### Specific Examples of Hole Transporting Zone Material

Specific examples of the hole transporting zone material according to the fourth, fifth, and sixth exemplary embodiments include the compounds listed in, for instance, the “common arrangement (3) among all the exemplary embodiments” described later.

### Acceptor Material

In an exemplary arrangement of the organic EL devices according to the fourth, fifth, and sixth exemplary embodiments, an acceptor material has at least one of a first cyclic structure represented by a formula (P11) below or a second cyclic structure represented by a formula (P12) below.

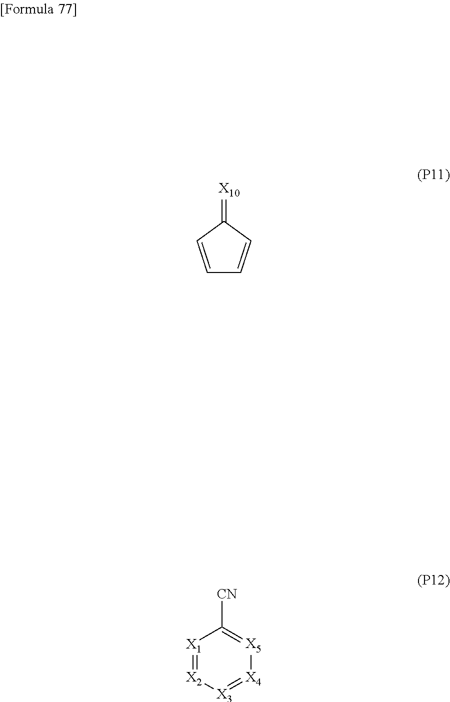

The first cyclic structure represented by the formula (P11) is fused, in a molecule of the acceptor material, to at least one cyclic structure of a substituted or unsubstituted aromatic hydrocarbon ring having 6 to 50 ring carbon atoms or a substituted or unsubstituted heterocycle having 5 to 50 ring atoms, and a structure represented by =X₁₀ is represented by a formula (11a), (11b), (11c), (11d), (11e), (11f), (11g), (11h), (11i), (11j), (11k) or (11m) below.

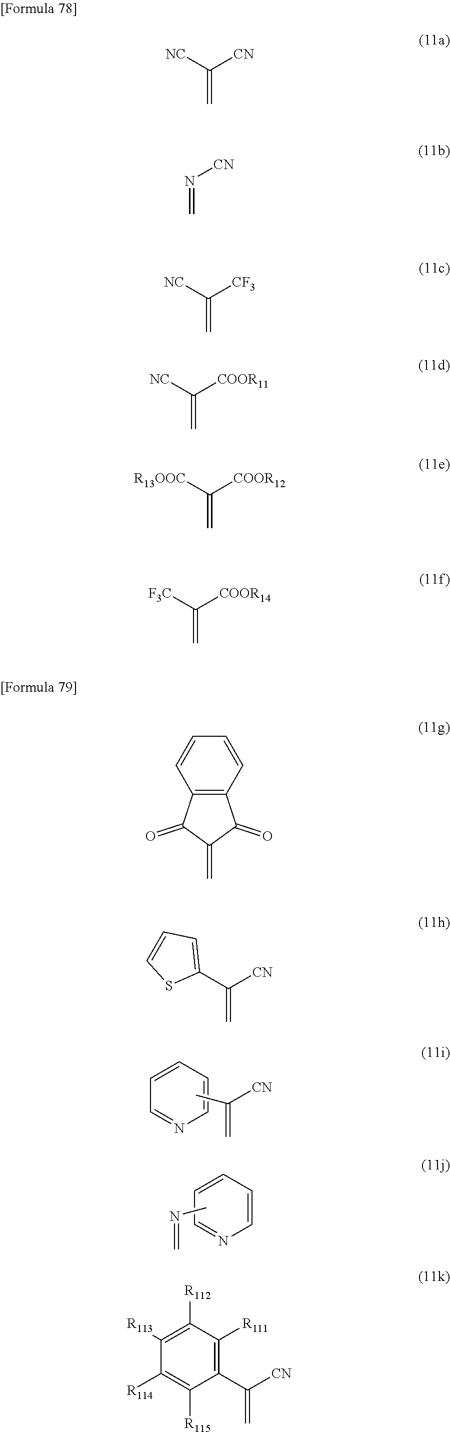

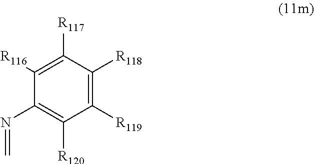

In the formula (11a), (11b), (11c), (11d), (11e), (11f), (11g), (11h), (11i), (11j), (11k) or (11m):
R₁₁ to R₁₄ and R₁₁₁ to R₁₂₀ are each independently a hydrogen atom, a halogen atom, a hydroxy group, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms.

In the formula (P12):
X₁ to X₅ are each independently a nitrogen atom, a carbon atom bonded to R₁₅, or a carbon atom bonded to another atom in a molecule of the acceptor material;
at least one of X₁ to X₅ is a carbon atom bonded to another atom in the molecule of the acceptor material;
R₁₅ is selected from the group consisting of a hydrogen atom, a halogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted alkenyl group having 2 to 50 carbon atoms, a substituted or unsubstituted aralkyl group having 7 to 50 carbon atoms, a carboxy group, a substituted or unsubstituted ester group, a substituted or unsubstituted carbamoyl group, a nitro group, and a substituted or unsubstituted siloxanyl group; and
when a plurality of R₁₅ are present, the plurality of R₁₅ are mutually the same or different.

In the acceptor material, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL devices of the fourth, fifth, and sixth exemplary embodiments, the acceptor material has at least one cyano group.

In an exemplary arrangement of the organic EL devices of the fourth, fifth, and sixth exemplary embodiments, the first anode-side organic layer contains the first organic material. The acceptor and the first organic material are mutually different.

In an exemplary arrangement of the organic EL devices of the fourth, fifth, and sixth exemplary embodiments, a content of the acceptor material in the first anode-side organic layer is less than 50 mass %.

In an exemplary arrangement of the organic EL devices of the fourth, fifth, and sixth exemplary embodiments, the content of the acceptor material in the first anode-side organic layer is 10 mass % or less, or 5 mass % or less.

In an exemplary arrangement of the organic EL devices of the fourth, fifth, and sixth exemplary embodiments, the content of the acceptor material in the first anode-side organic layer is in a range from 1 mass % to 3 mass %.

In an exemplary arrangement of the organic EL devices of the fourth, fifth, and sixth exemplary embodiments, the acceptor material and the first organic material are compounds different from the third organic material.

In an exemplary arrangement of the organic EL devices of the fourth, fifth, and sixth exemplary embodiments, the first organic material and the second organic material may be mutually different compounds or an identical compound.

In an exemplary arrangement of the organic EL devices of the fourth, fifth, and sixth exemplary embodiments, in a case where the first anode-side organic layer contains the acceptor material and the first organic material, a content of the first organic material in the first anode-side organic layer is preferably 40 mass % or more, more preferably 45 mass % or more, and still more preferably 50 mass % or more. The content of the first organic material in the first anode-side organic layer is preferably 99.5 mass % or less. The total of the content of the acceptor material and the content of the first organic material in the first anode-side organic layer is 100 mass % or less.

In the acceptor material, the groups specified to be “substituted or unsubstituted” are each preferably an “unsubstituted” group.

Specific examples of the acceptor material include the compounds listed in, for instance, the “common arrangement (3) among all the exemplary embodiments” described later.

### Additive Material

In an exemplary arrangement of the organic EL devices of the fourth, fifth, and sixth exemplary embodiments, the fourth material, the fifth material, and the sixth material as the additive material are each independently an organic material or a metal atom-containing material.

In an exemplary arrangement of the organic EL devices of the fourth, fifth, and sixth exemplary embodiments, the fourth material as the additive material is a material such that the refractive index of the second anode-side organic layer satisfies both of the numerical formulae (Numerical Formula NX1 and Numerical Formula NX2).

In an exemplary arrangement of the organic EL devices of the fourth, fifth, and sixth exemplary embodiments, the fifth material as the additive material is a material such that the refractive index of the third anode-side organic layer satisfies both of the numerical formulae (Numerical Formula NX1 and Numerical Formula NX3).

In an exemplary arrangement of the organic EL devices of the fourth, fifth, and sixth exemplary embodiments, the refractive index of the fifth material is 1.80 or less.

In an exemplary arrangement of the organic EL devices of the fourth, fifth, and sixth exemplary embodiments, the refractive index of the fifth material is 1.78 or less.

In an exemplary arrangement of the organic EL devices of the fourth, fifth, and sixth exemplary embodiments, the sixth material as the additive material is a material that causes the refractive index of the first anode-side organic layer to satisfy the numerical formula (Numerical Formula NX6).

In an exemplary arrangement of the organic EL devices of the fourth, fifth, and sixth exemplary embodiments, the additive material is an organic material.

In an exemplary arrangement of the organic EL devices of the fourth, fifth, and sixth exemplary embodiments, the organic material as the additive material is an amine compound having at least one substituted or unsubstituted amino group in a molecule, or a non-amine compound not having a substituted or unsubstituted amino group in a molecule.

In an exemplary arrangement of the organic EL devices of the fourth, fifth, and sixth exemplary embodiments, the organic material as the additive material is an amine compound having a thiophene ring.

In an exemplary arrangement of the organic EL devices of the fourth, fifth, and sixth exemplary embodiments, in a case where an amine compound as the additive material is contained in the anode-side organic layers of the first hole transporting zone, a content of the amine compound in each of the anode-side organic layers is preferably 10 mass % or more, more preferably 20 mass % or more.

In an exemplary arrangement of the organic EL devices of the fourth, fifth, and sixth exemplary embodiments, in a case where the amine compound as the additive material is contained in the anode-side organic layers of the first hole transporting zone, the content of the amine compound in each of the anode-side organic layers is preferably 50 mass % or less.

In an exemplary arrangement of the organic EL devices of the fourth, fifth, and sixth exemplary embodiments, in a case where a non-amine compound as the additive material is contained in the anode-side organic layers of the first hole transporting zone, a content of the non-amine compound in each of the anode-side organic layers is preferably 5 mass % or more, more preferably 10 mass % or more, and still more preferably 20 mass % or more.

In an exemplary arrangement of the organic EL devices of the fourth, fifth, and sixth exemplary embodiments, in a case where the non-amine compound as the additive material is contained in the anode-side organic layers of the first hole transporting zone, the content of the non-amine compound in each of the anode-side organic layers is preferably 50 mass % or less.

Specific examples of the fourth material and the six material as the additive material in an exemplary arrangement of the organic EL devices of the fourth, fifth, and sixth exemplary embodiments include the compounds listed in, for instance, the “common arrangement (3) among all the exemplary embodiments” described later.

In an exemplary arrangement of the organic EL devices of the fourth, fifth, and sixth exemplary embodiments, the additive material is a metal atom-containing material.

In an exemplary arrangement of the organic EL devices of the fourth, fifth, and sixth exemplary embodiments, the metal atom-containing material is exemplified by a compound having a perovskite structure (perovskite compound). In an exemplary arrangement of the organic EL devices of the fourth, fifth, and sixth exemplary embodiments, a perovskite compound as the metal atom-containing material is represented by a formula ABX₃ in which A and B are each a cation and X is an anion. For instance, A is CH₃NH₃ (“CH₃NH₃” is sometimes referred to as “MA” hereinafter), CH(NH₂)₂, Cs, or a mixture thereof; B is Pb, Sn, or a mixture thereof; and X is Cl, Br, I, or a mixture thereof. The perovskite compound as the metal atom-containing material is exemplified by CH₃NH₃PbBr₃. A refractive index of CH₃NH₃PbBr₃ at 460 nm is 2.3.

In an exemplary arrangement of the organic EL devices of the fourth, fifth, and sixth exemplary embodiments, the metal atom-containing material is a metal oxide. In an exemplary arrangement of the organic EL devices of the fourth, fifth, and sixth exemplary embodiments, the metal oxide as the metal atom-containing material is exemplified by zinc oxide (ZnO).

In an exemplary arrangement of the organic EL devices of the fourth, fifth, and sixth exemplary embodiments, the fourth material as the additive material is a perovskite compound or zinc oxide (ZnO).

In an exemplary arrangement of the organic EL devices of the fourth, fifth, and sixth exemplary embodiments, the sixth material as the additive material is a perovskite compound or zinc oxide (ZnO).

Specific examples of the fifth material as the additive material in an exemplary arrangement of the organic EL devices of the fourth, fifth, and sixth exemplary embodiments include the compounds listed in, for instance, the “common arrangement (3) among all the exemplary embodiments” described later.

### Common Arrangement (3) Among all Exemplary Embodiments

Regarding the common arrangement (3) among all the exemplary embodiments, an arrangement applicable to each of the exemplary embodiments (the first exemplary embodiment, the second exemplary embodiment, the third exemplary embodiment, the fourth exemplary embodiment, the fifth exemplary embodiment, and the sixth exemplary embodiment) described herein and modifications of each of the exemplary embodiments will be described below.

In the monoamine compound and the diamine compound, a nitrogen atom of an amino group is not a ring atom. When a nitrogen atom is a ring atom in a carbazole ring, an azine ring, and the like, the nitrogen atom is not a nitrogen atom as an amino group.

For instance, a compound HT-X below has two nitrogen atoms in a molecule: one nitrogen atom in the compound HT-X is a ring atom of a carbazole ring and the other nitrogen atom is not a ring atom but a nitrogen atom as an amino group. The compound HT-X is a compound having a structure in which a 9-phenyl-3-carbazolyl group is bonded to a nitrogen atom of an amino group via a linking group, that is, a monoamine compound.

A compound HT-Y below is also a compound having a structure in which a 9-carbazolyl group is bonded to a nitrogen atom of an amino group via a linking group, that is, a monoamine compound.

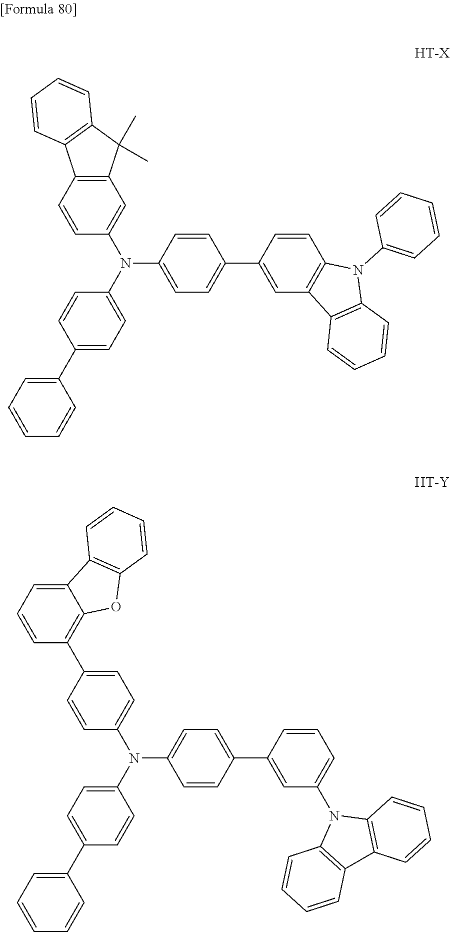 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

### Specific Examples of Hole Transporting Zone Material

Specific examples of the hole transporting zone material according to each of the exemplary embodiments include compounds below. However, the invention is by no means limited to the specific examples.

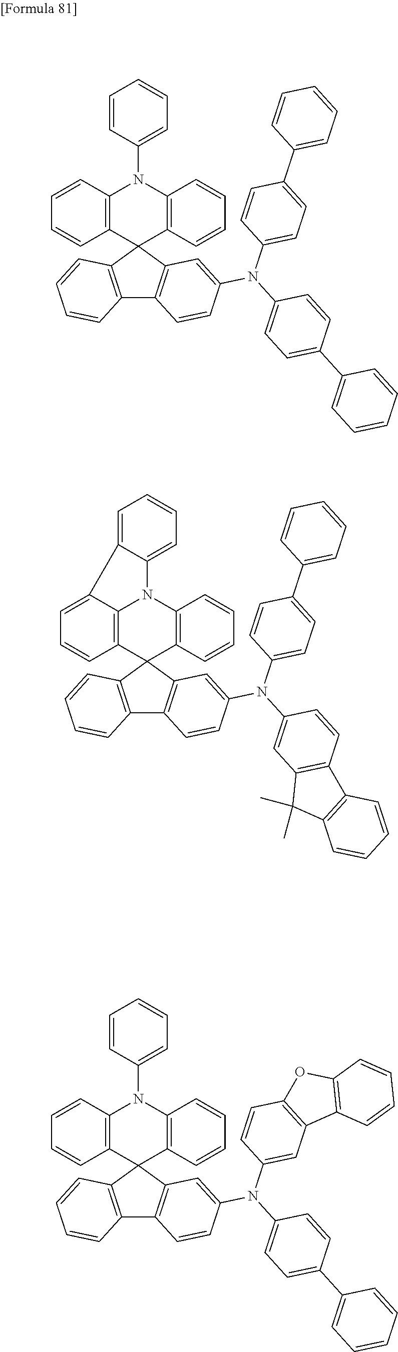 [SMILES: c1ccc(-c2ccc(N(c3ccc(-c4ccccc4)cc3)c3ccc4c(c3)C3(c5ccccc5-4)c4ccccc4N(c4ccccc4)c4ccccc43)cc2)cc1 ; CC1(C)c2ccccc2-c2ccc(N(c3ccc(-c4ccccc4)cc3)c3ccc4c(c3)C3(c5ccccc5-4)c4ccccc4-n4c5ccccc5c5cccc3c54)cc21 ; c1ccc(-c2ccc(N(c3ccc4c(c3)C3(c5ccccc5-4)c4ccccc4N(c4ccccc4)c4ccccc43)c3ccc4oc5ccccc5c4c3)cc2)cc1]

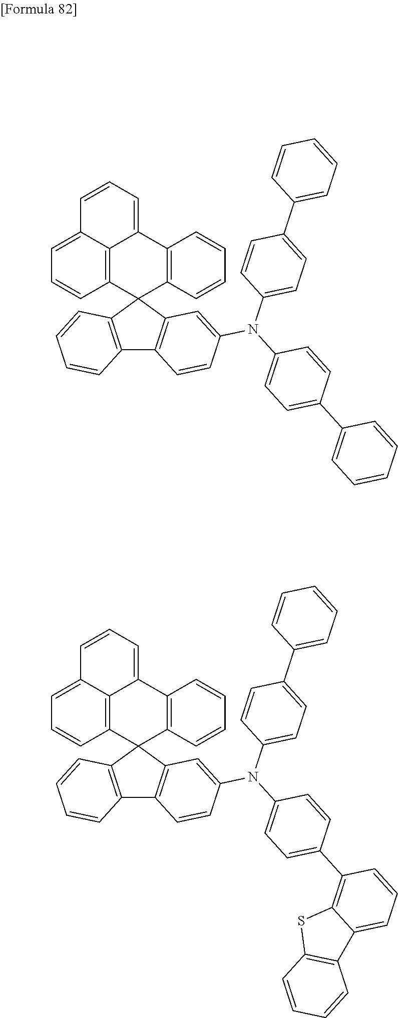 [SMILES: c1ccc(-c2ccc(N(c3ccc(-c4ccccc4)cc3)c3ccc4c(c3)C3(c5ccccc5-4)c4ccccc4-c4cccc5cccc3c45)cc2)cc1 ; c1ccc(-c2ccc(N(c3ccc(-c4cccc5c4sc4ccccc45)cc3)c3ccc4c(c3)C3(c5ccccc5-4)c4ccccc4-c4cccc5cccc3c45)cc2)cc1]

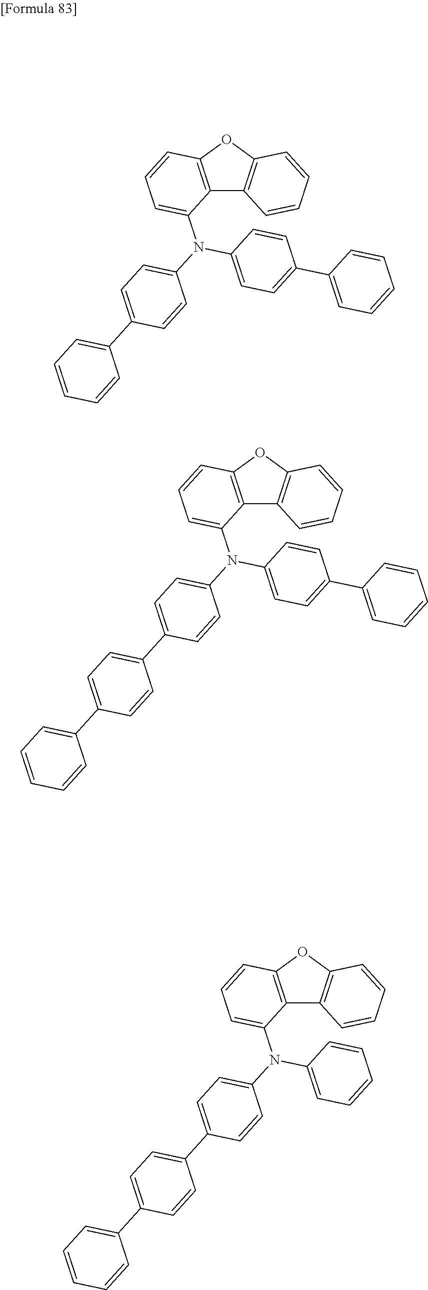 [SMILES: c1ccc(-c2ccc(N(c3ccc(-c4ccccc4)cc3)c3cccc4oc5ccccc5c34)cc2)cc1 ; c1ccc(-c2ccc(-c3ccc(N(c4ccc(-c5ccccc5)cc4)c4cccc5oc6ccccc6c45)cc3)cc2)cc1 ; c1ccc(-c2ccc(-c3ccc(N(c4ccccc4)c4cccc5oc6ccccc6c45)cc3)cc2)cc1]

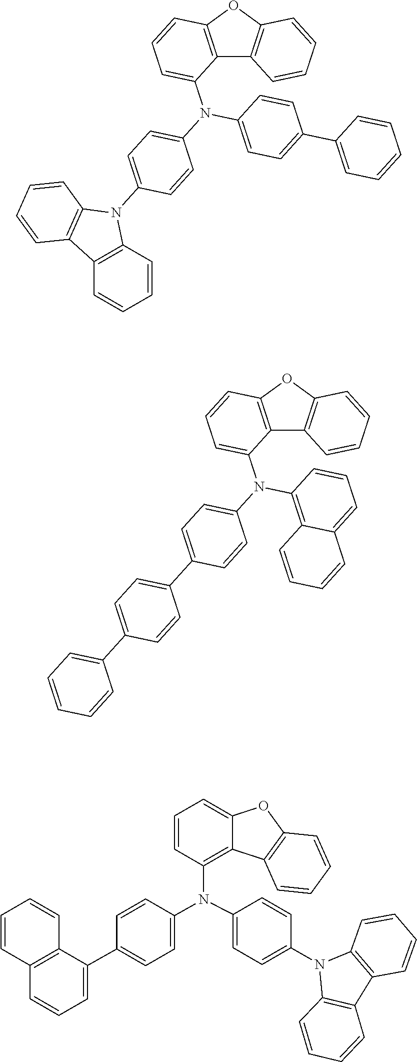 [SMILES: c1ccc(-c2ccc(N(c3ccc(-n4c5ccccc5c5ccccc54)cc3)c3cccc4oc5ccccc5c34)cc2)cc1 ; c1ccc(-c2ccc(-c3ccc(N(c4cccc5ccccc45)c4cccc5oc6ccccc6c45)cc3)cc2)cc1 ; c1ccc2c(-c3ccc(N(c4ccc(-n5c6ccccc6c6ccccc65)cc4)c4cccc5oc6ccccc6c45)cc3)cccc2c1]

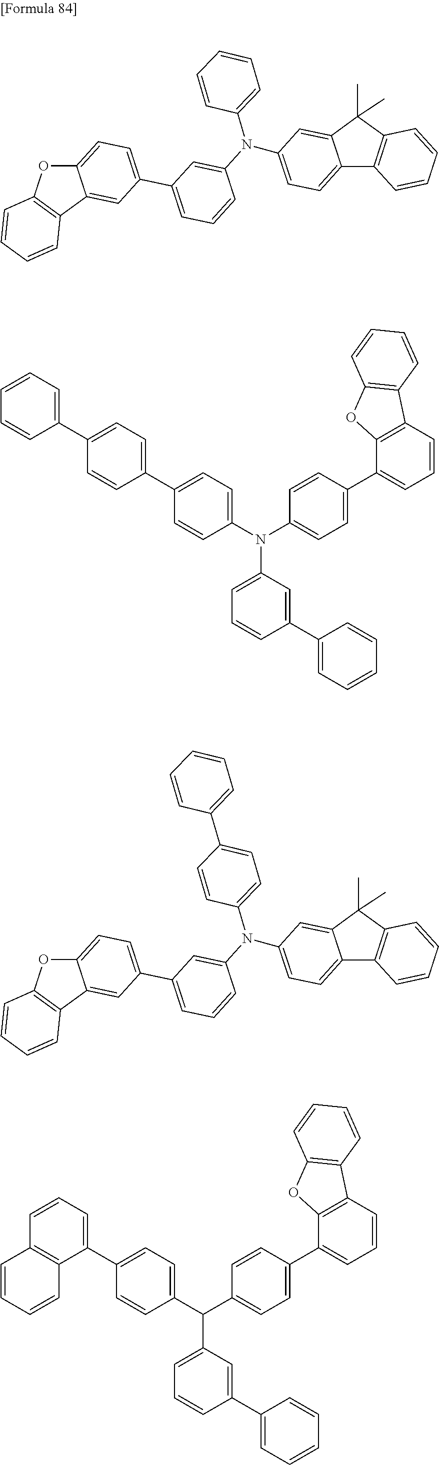

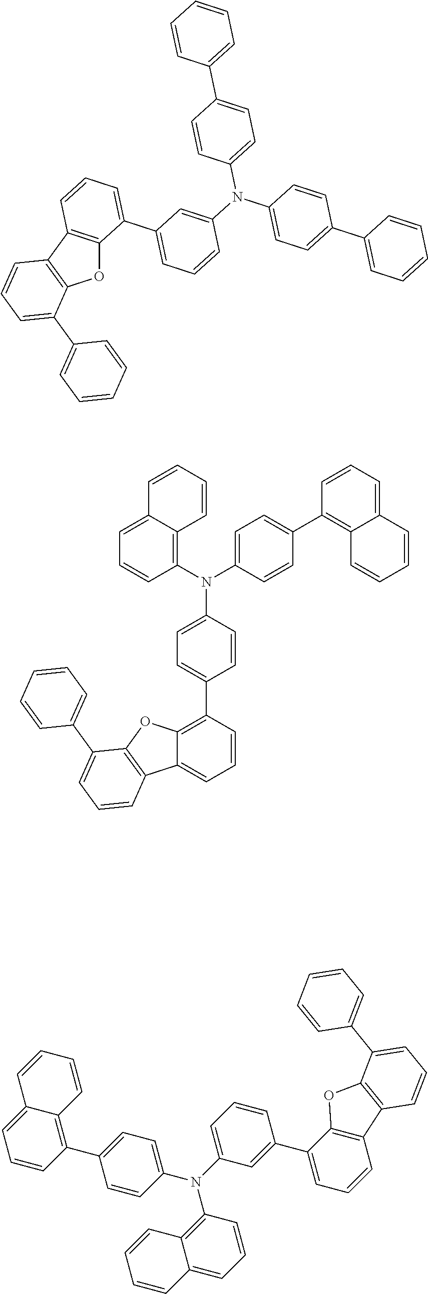 [SMILES: c1ccc(-c2ccc(N(c3ccc(-c4ccccc4)cc3)c3cccc(-c4cccc5c4oc4c(-c6ccccc6)cccc45)c3)cc2)cc1 ; c1ccc(-c2cccc3c2oc2c(-c4ccc(N(c5ccc(-c6cccc7ccccc67)cc5)c5cccc6ccccc56)cc4)cccc23)cc1 ; c1ccc(-c2cccc3c2oc2c(-c4cccc(N(c5ccc(-c6cccc7ccccc67)cc5)c5cccc6ccccc56)c4)cccc23)cc1]

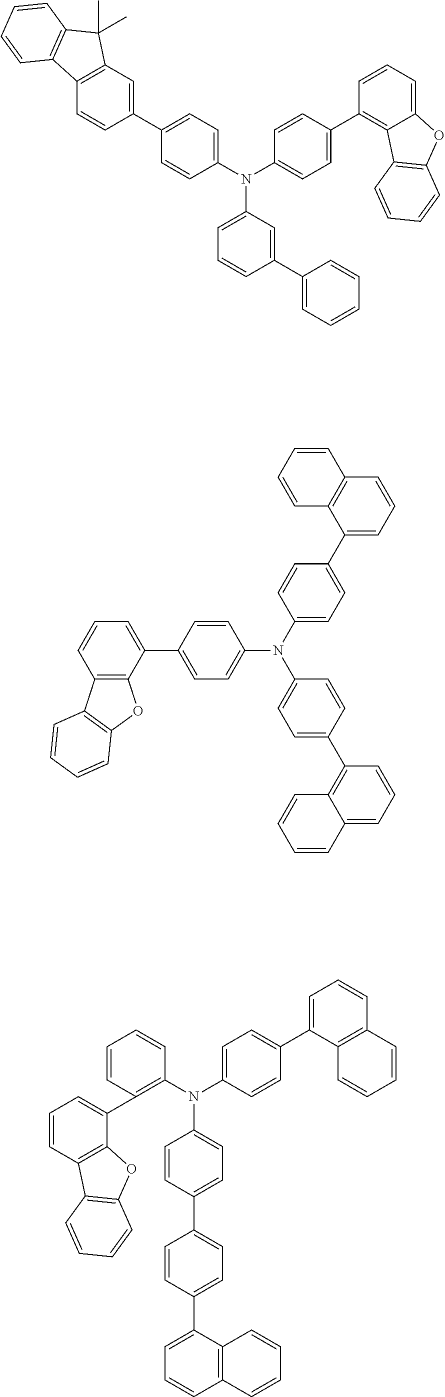 [SMILES: CC1(C)c2ccccc2-c2ccc(-c3ccc(N(c4ccc(-c5cccc6oc7ccccc7c56)cc4)c4cccc(-c5ccccc5)c4)cc3)cc21 ; c1ccc2c(-c3ccc(N(c4ccc(-c5cccc6ccccc56)cc4)c4ccc(-c5cccc6c5oc5ccccc56)cc4)cc3)cccc2c1 ; c1ccc(N(c2ccc(-c3ccc(-c4cccc5ccccc45)cc3)cc2)c2ccc(-c3cccc4ccccc34)cc2)c(-c2cccc3c2oc2ccccc23)c1]

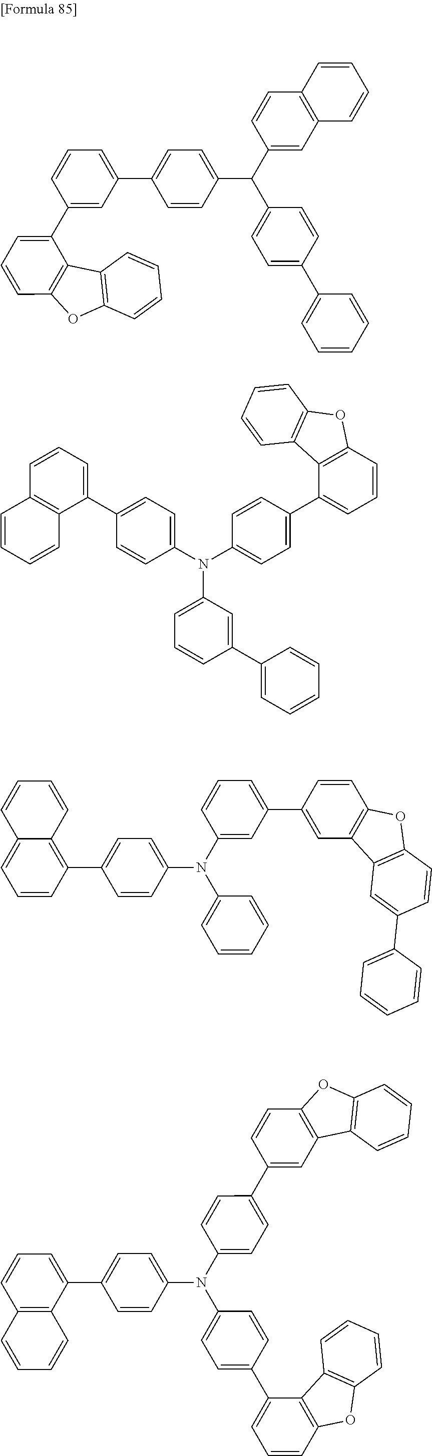

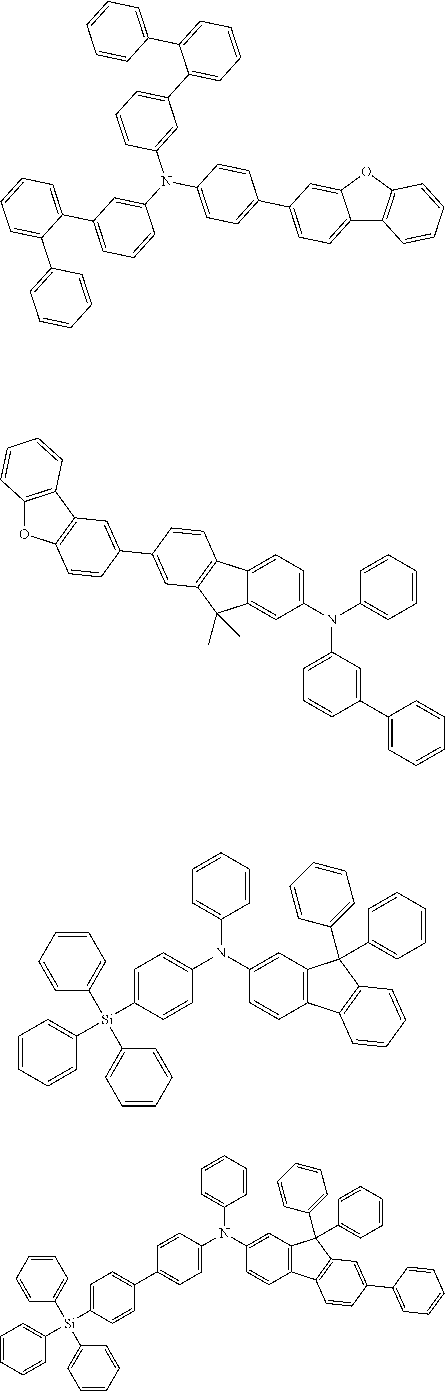 [SMILES: c1ccc(-c2ccccc2-c2cccc(N(c3ccc(-c4ccc5c(c4)oc4ccccc45)cc3)c3cccc(-c4ccccc4-c4ccccc4)c3)c2)cc1 ; CC1(C)c2cc(-c3ccc4oc5ccccc5c4c3)ccc2-c2ccc(N(c3ccccc3)c3cccc(-c4ccccc4)c3)cc21 ; c1ccc(N(c2ccc([Si](c3ccccc3)(c3ccccc3)c3ccccc3)cc2)c2ccc3c(c2)C(c2ccccc2)(c2ccccc2)c2ccccc2-3)cc1 ; c1ccc(-c2ccc3c(c2)C(c2ccccc2)(c2ccccc2)c2cc(N(c4ccccc4)c4ccc(-c5ccc([Si](c6ccccc6)(c6ccccc6)c6ccccc6)cc5)cc4)ccc2-3)cc1]

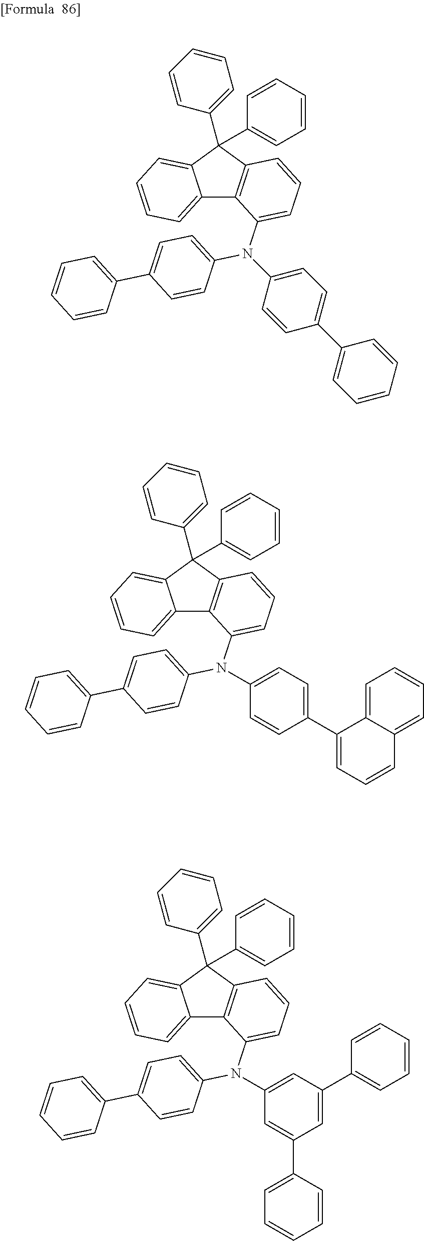 [SMILES: c1ccc(-c2ccc(N(c3ccc(-c4ccccc4)cc3)c3cccc4c3-c3ccccc3C4(c3ccccc3)c3ccccc3)cc2)cc1 ; c1ccc(-c2ccc(N(c3ccc(-c4cccc5ccccc45)cc3)c3cccc4c3-c3ccccc3C4(c3ccccc3)c3ccccc3)cc2)cc1 ; c1ccc(-c2ccc(N(c3cc(-c4ccccc4)cc(-c4ccccc4)c3)c3cccc4c3-c3ccccc3C4(c3ccccc3)c3ccccc3)cc2)cc1]

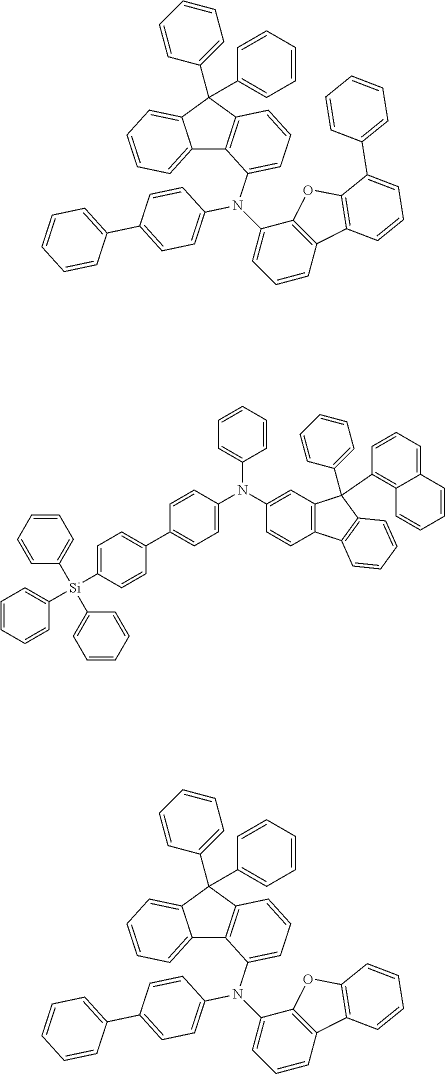 [SMILES: c1ccc(-c2ccc(N(c3cccc4c3-c3ccccc3C4(c3ccccc3)c3ccccc3)c3cccc4c3oc3c(-c5ccccc5)cccc34)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2cccc4ccccc24)c2ccccc2-3)cc1 ; c1ccc(-c2ccc(N(c3cccc4c3-c3ccccc3C4(c3ccccc3)c3ccccc3)c3cccc4c3oc3ccccc34)cc2)cc1]

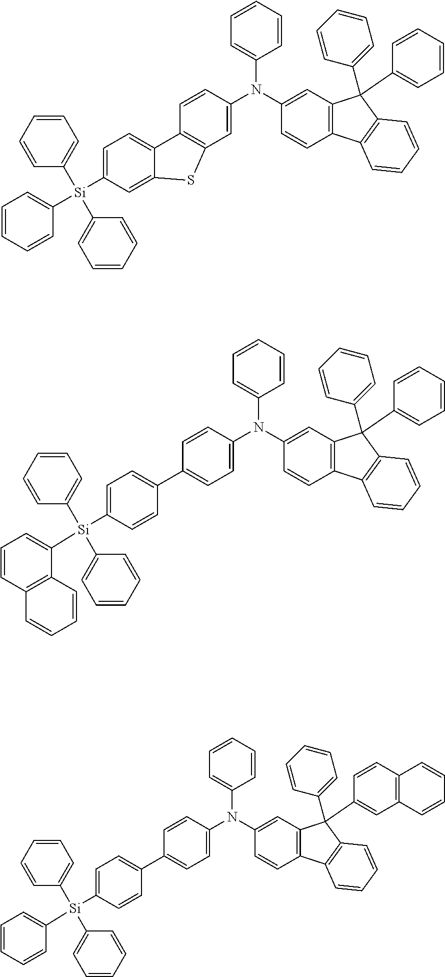 [SMILES: c1ccc(N(c2ccc3c(c2)C(c2ccccc2)(c2ccccc2)c2ccccc2-3)c2ccc3c(c2)sc2cc([Si](c4ccccc4)(c4ccccc4)c4ccccc4)ccc23)cc1 ; c1ccc(N(c2ccc(-c3ccc([Si](c4ccccc4)(c4ccccc4)c4cccc5ccccc45)cc3)cc2)c2ccc3c(c2)C(c2ccccc2)(c2ccccc2)c2ccccc2-3)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccc4ccccc4c2)c2ccccc2-3)cc1]

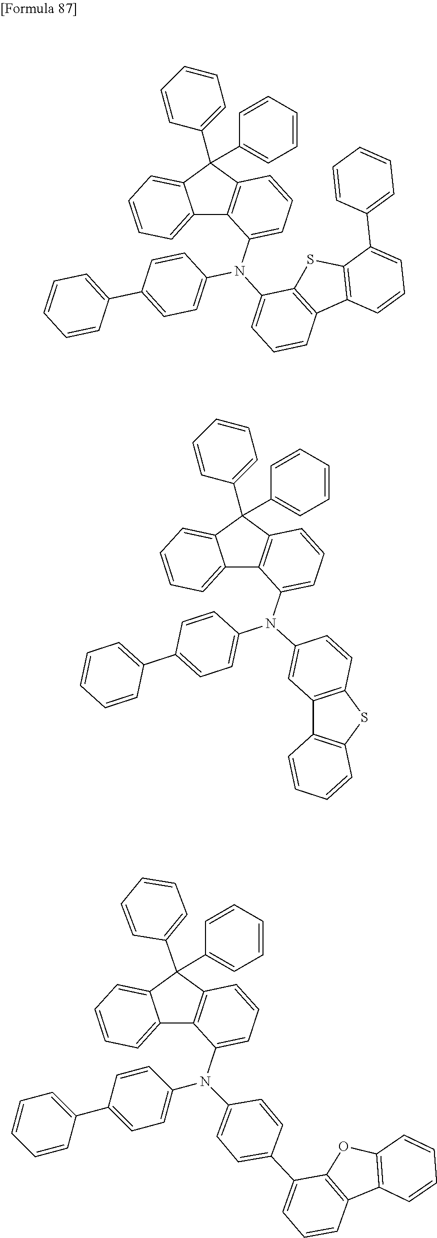 [SMILES: c1ccc(-c2ccc(N(c3cccc4c3-c3ccccc3C4(c3ccccc3)c3ccccc3)c3cccc4c3sc3c(-c5ccccc5)cccc34)cc2)cc1 ; c1ccc(-c2ccc(N(c3ccc4sc5ccccc5c4c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3cccc4c3-c3ccccc3C4(c3ccccc3)c3ccccc3)cc2)cc1]

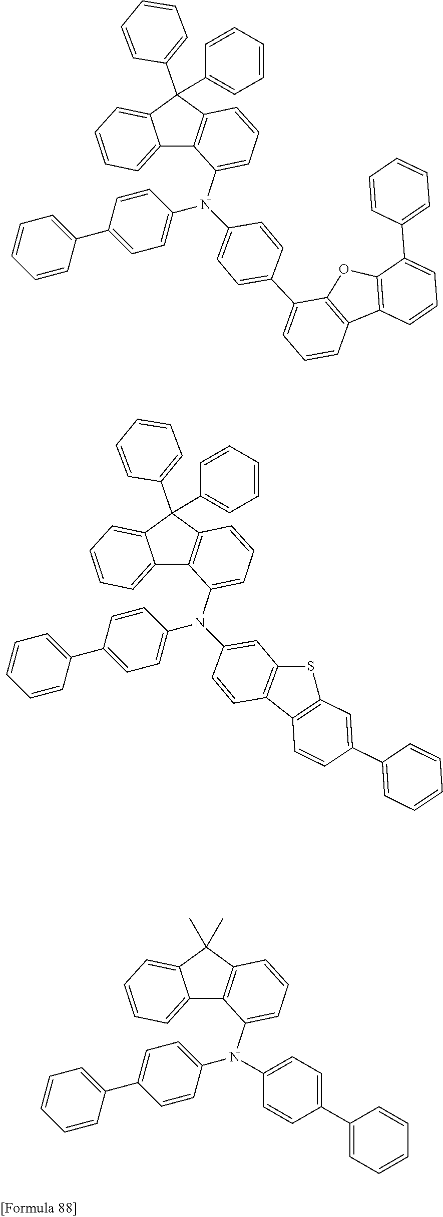 [SMILES: c1ccc(-c2ccc(N(c3ccc(-c4cccc5c4oc4c(-c6ccccc6)cccc45)cc3)c3cccc4c3-c3ccccc3C4(c3ccccc3)c3ccccc3)cc2)cc1 ; c1ccc(-c2ccc(N(c3ccc4c(c3)sc3cc(-c5ccccc5)ccc34)c3cccc4c3-c3ccccc3C4(c3ccccc3)c3ccccc3)cc2)cc1 ; CC1(C)c2ccccc2-c2c(N(c3ccc(-c4ccccc4)cc3)c3ccc(-c4ccccc4)cc3)cccc21]

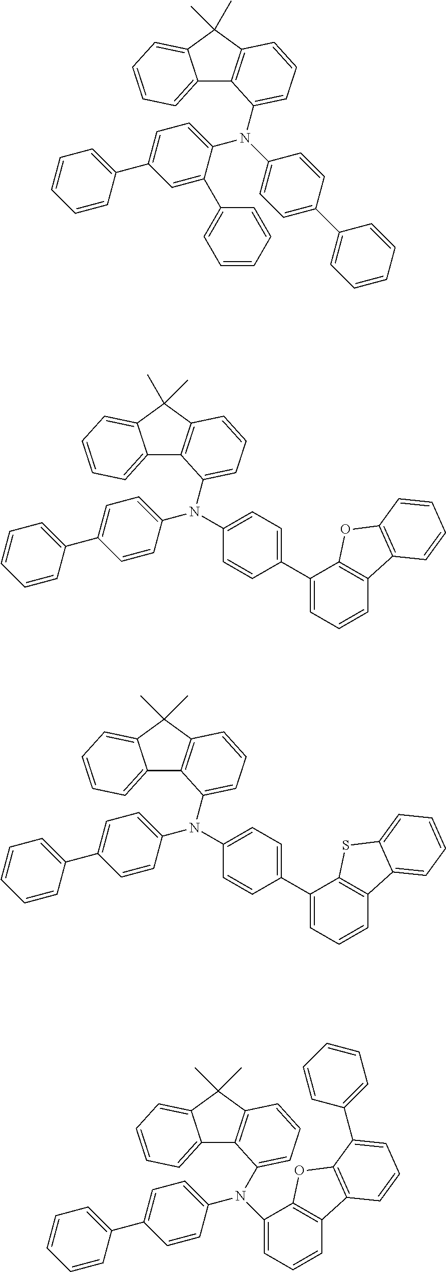 [SMILES: CC1(C)c2ccccc2-c2c(N(c3ccc(-c4ccccc4)cc3)c3ccc(-c4ccccc4)cc3-c3ccccc3)cccc21 ; CC1(C)c2ccccc2-c2c(N(c3ccc(-c4ccccc4)cc3)c3ccc(-c4cccc5c4oc4ccccc45)cc3)cccc21 ; CC1(C)c2ccccc2-c2c(N(c3ccc(-c4ccccc4)cc3)c3ccc(-c4cccc5c4sc4ccccc45)cc3)cccc21 ; CC1(C)c2ccccc2-c2c(N(c3ccc(-c4ccccc4)cc3)c3cccc4c3oc3c(-c5ccccc5)cccc34)cccc21]

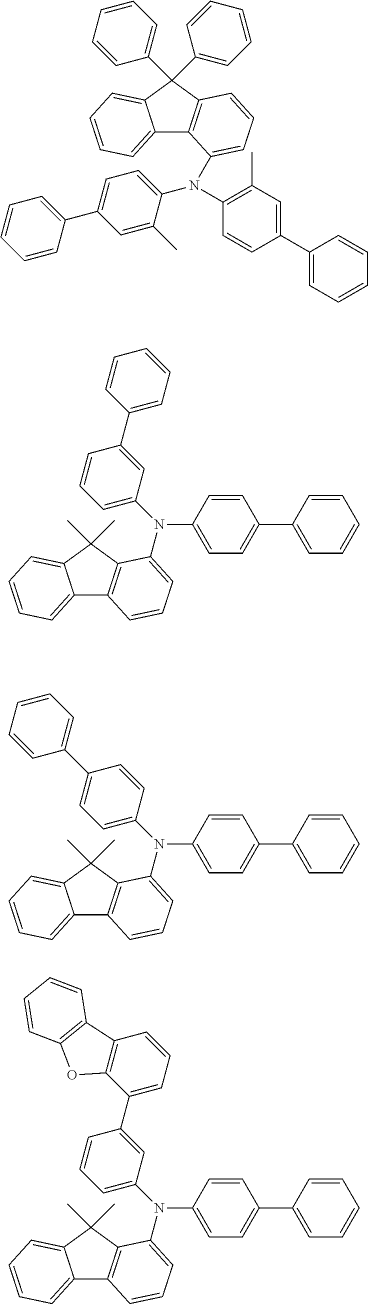

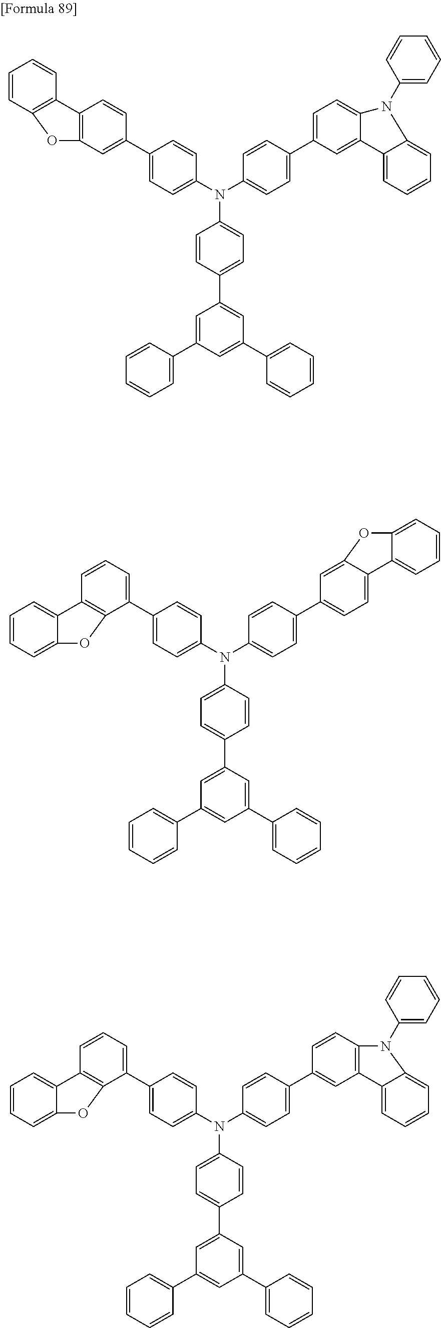 [SMILES: c1ccc(-c2cc(-c3ccccc3)cc(-c3ccc(N(c4ccc(-c5ccc6c(c5)oc5ccccc56)cc4)c4ccc(-c5ccc6c(c5)c5ccccc5n6-c5ccccc5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5cccc6c5oc5ccccc56)cc4)cc3)c2)cc1 ; c1ccc(-c2cc(-c3ccccc3)cc(-c3ccc(N(c4ccc(-c5ccc6c(c5)c5ccccc5n6-c5ccccc5)cc4)c4ccc(-c5cccc6c5oc5ccccc56)cc4)cc3)c2)cc1]

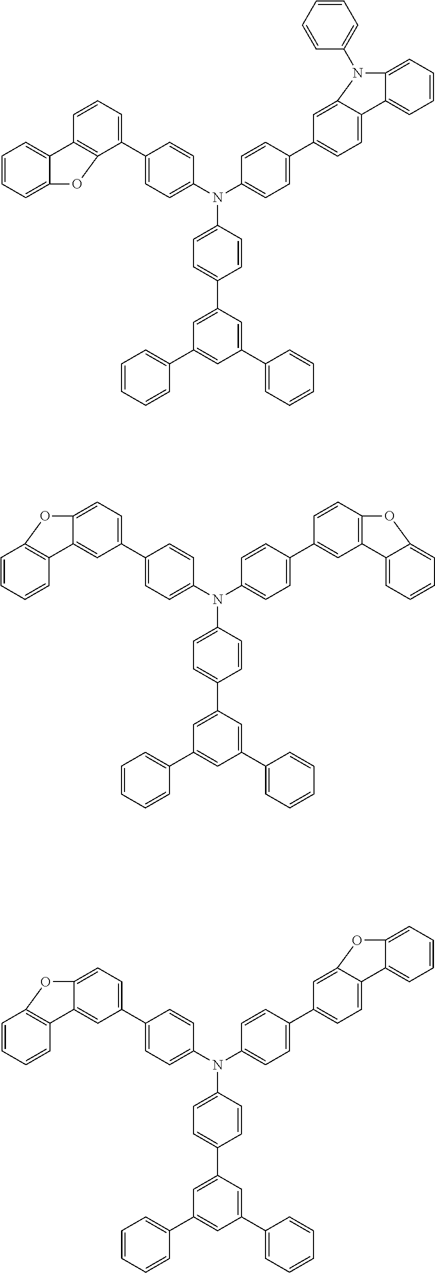 [SMILES: c1ccc(-c2cc(-c3ccccc3)cc(-c3ccc(N(c4ccc(-c5ccc6c7ccccc7n(-c7ccccc7)c6c5)cc4)c4ccc(-c5cccc6c5oc5ccccc56)cc4)cc3)c2)cc1 ; c1ccc(-c2cc(-c3ccccc3)cc(-c3ccc(N(c4ccc(-c5ccc6oc7ccccc7c6c5)cc4)c4ccc(-c5ccc6oc7ccccc7c6c5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5ccc6oc7ccccc7c6c5)cc4)cc3)c2)cc1]

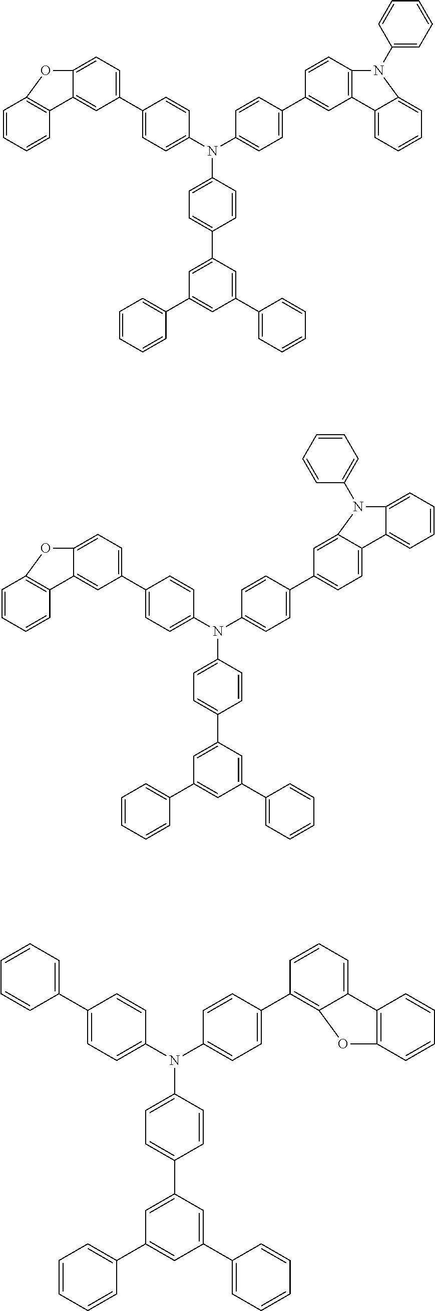 [SMILES: c1ccc(-c2cc(-c3ccccc3)cc(-c3ccc(N(c4ccc(-c5ccc6oc7ccccc7c6c5)cc4)c4ccc(-c5ccc6c(c5)c5ccccc5n6-c5ccccc5)cc4)cc3)c2)cc1 ; c1ccc(-c2cc(-c3ccccc3)cc(-c3ccc(N(c4ccc(-c5ccc6oc7ccccc7c6c5)cc4)c4ccc(-c5ccc6c7ccccc7n(-c7ccccc7)c6c5)cc4)cc3)c2)cc1 ; c1ccc(-c2ccc(N(c3ccc(-c4cc(-c5ccccc5)cc(-c5ccccc5)c4)cc3)c3ccc(-c4cccc5c4oc4ccccc45)cc3)cc2)cc1]

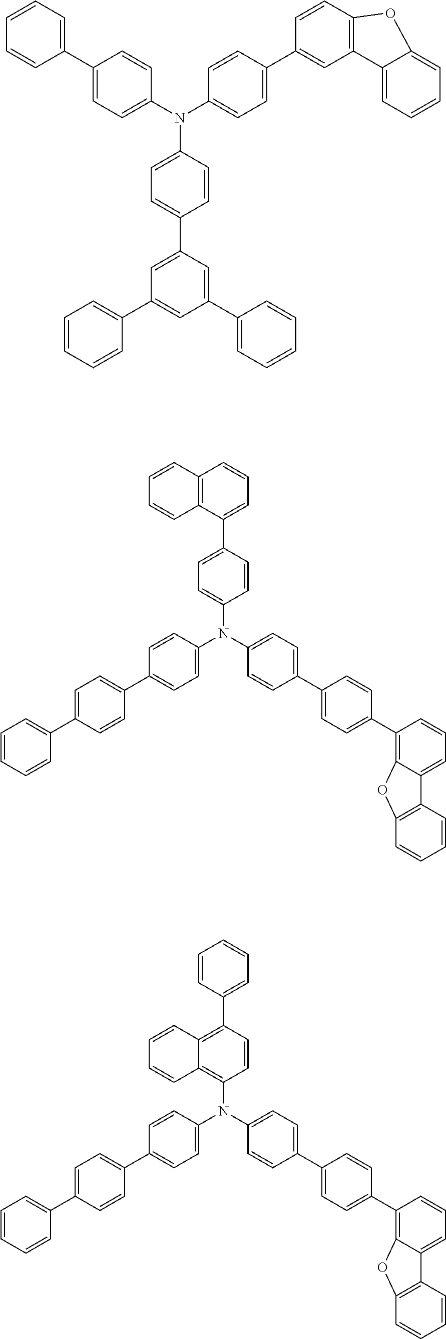 [SMILES: c1ccc(-c2ccc(N(c3ccc(-c4cc(-c5ccccc5)cc(-c5ccccc5)c4)cc3)c3ccc(-c4ccc5oc6ccccc6c5c4)cc3)cc2)cc1 ; c1ccc(-c2ccc(-c3ccc(N(c4ccc(-c5ccc(-c6cccc7c6oc6ccccc67)cc5)cc4)c4ccc(-c5cccc6ccccc56)cc4)cc3)cc2)cc1 ; c1ccc(-c2ccc(-c3ccc(N(c4ccc(-c5ccc(-c6cccc7c6oc6ccccc67)cc5)cc4)c4ccc(-c5ccccc5)c5ccccc45)cc3)cc2)cc1]

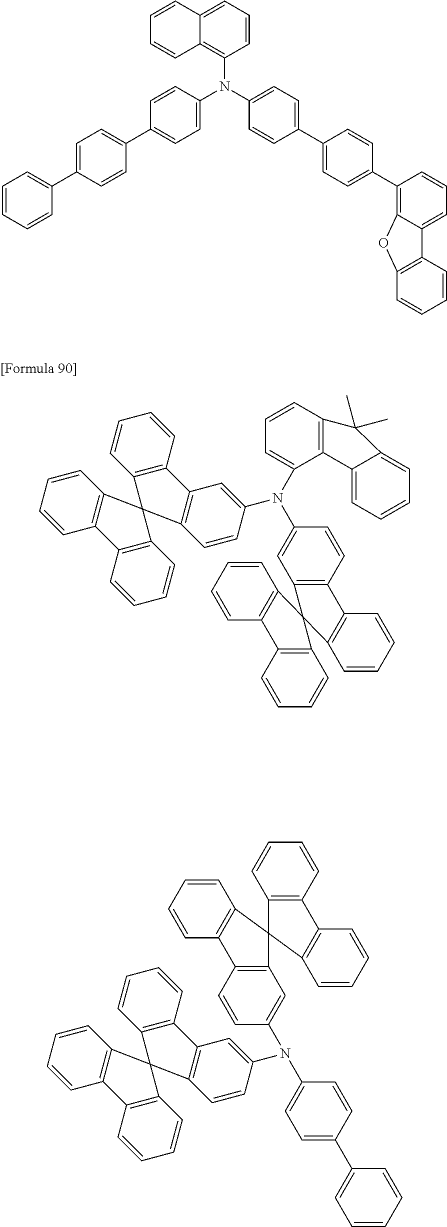 [SMILES: c1ccc(-c2ccc(-c3ccc(N(c4ccc(-c5ccc(-c6cccc7c6oc6ccccc67)cc5)cc4)c4cccc5ccccc45)cc3)cc2)cc1 ; CC1(C)c2ccccc2-c2c(N(c3ccc4c(c3)-c3ccccc3C43c4ccccc4-c4ccccc43)c3ccc4c(c3)C3(c5ccccc5-c5ccccc53)c3ccccc3-4)cccc21 ; c1ccc(-c2ccc(N(c3ccc4c(c3)-c3ccccc3C43c4ccccc4-c4ccccc43)c3ccc4c(c3)C3(c5ccccc5-c5ccccc53)c3ccccc3-4)cc2)cc1]

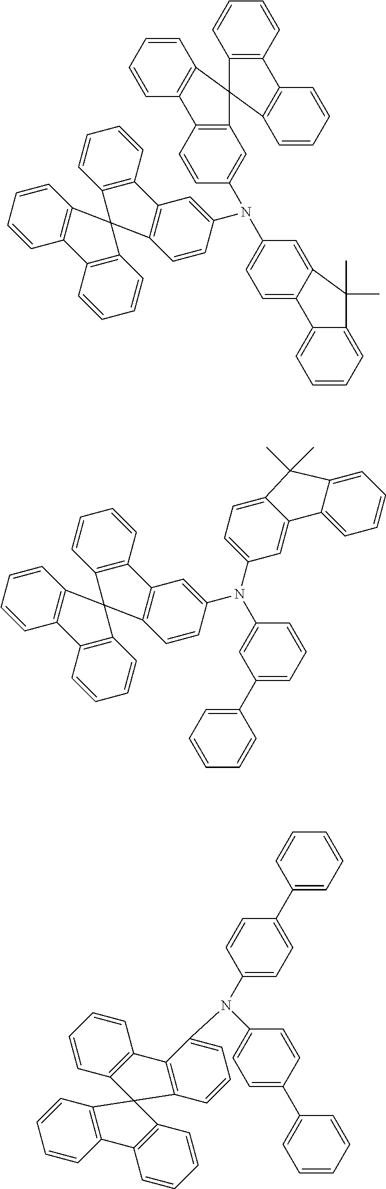 [SMILES: CC1(C)c2ccccc2-c2ccc(N(c3ccc4c(c3)-c3ccccc3C43c4ccccc4-c4ccccc43)c3ccc4c(c3)C3(c5ccccc5-c5ccccc53)c3ccccc3-4)cc21 ; CC1(C)c2ccccc2-c2cc(N(c3cccc(-c4ccccc4)c3)c3ccc4c(c3)-c3ccccc3C43c4ccccc4-c4ccccc43)ccc21 ; c1ccc(-c2ccc(N(c3ccc(-c4ccccc4)cc3)c3cccc4c3-c3ccccc3C43c4ccccc4-c4ccccc43)cc2)cc1]

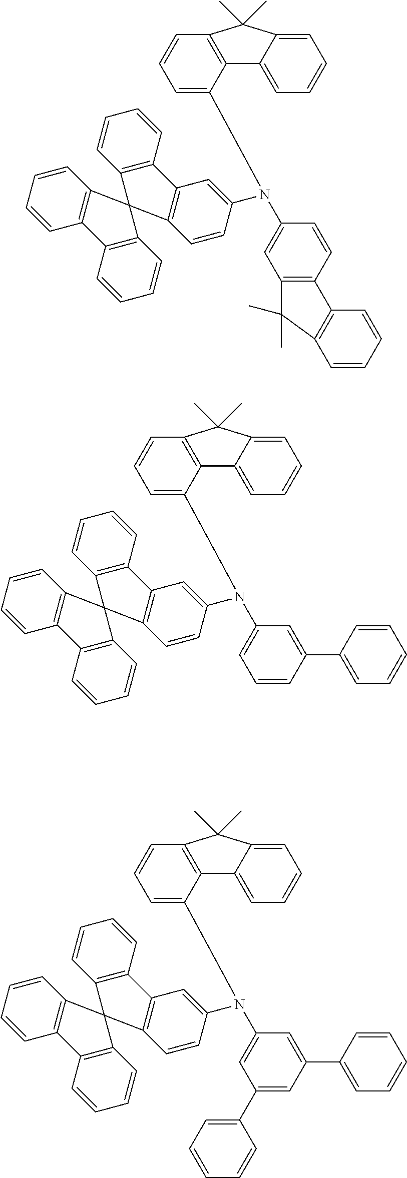 [SMILES: CC(C)(C)c1cc(N(c2ccc3c(c2)-c2ccccc2C32c3ccccc3-c3ccccc32)c2cccc3c2-c2ccccc2C3(C)C)ccc1-c1ccccc1 ; CC1(C)c2ccccc2-c2c(N(c3cccc(-c4ccccc4)c3)c3ccc4c(c3)-c3ccccc3C43c4ccccc4-c4ccccc43)cccc21 ; CC1(C)c2ccccc2-c2c(N(c3cc(-c4ccccc4)cc(-c4ccccc4)c3)c3ccc4c(c3)-c3ccccc3C43c4ccccc4-c4ccccc43)cccc21]

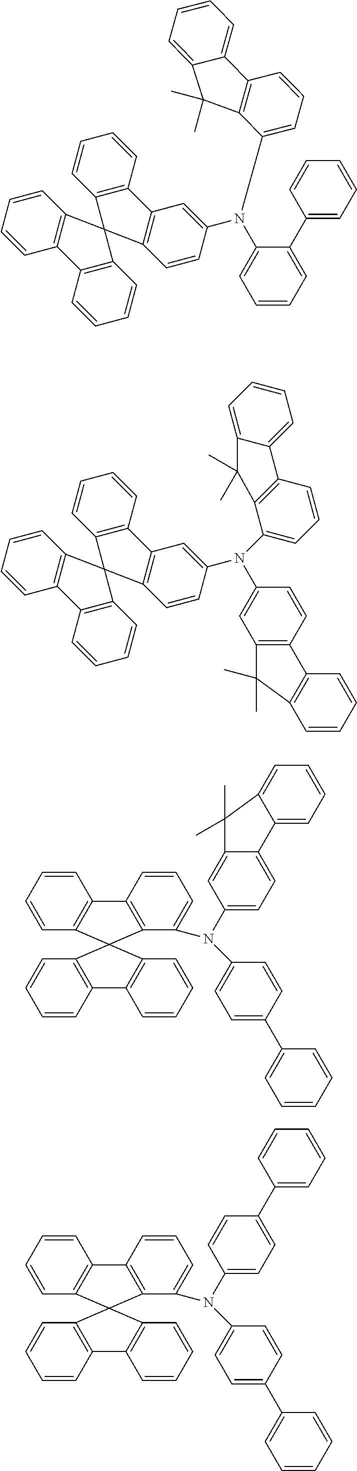

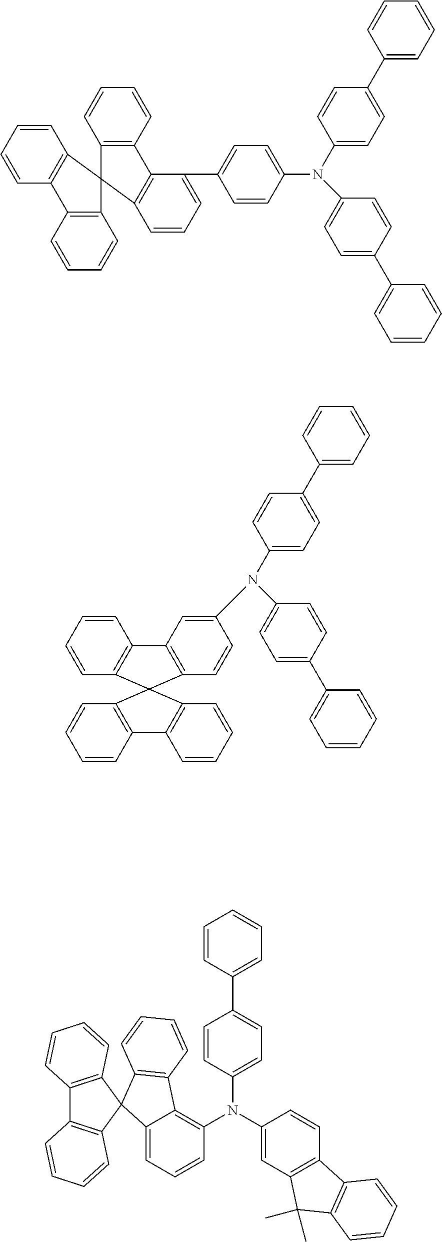 [SMILES: c1ccc(-c2ccc(N(c3ccc(-c4ccccc4)cc3)c3ccc(-c4cccc5c4-c4ccccc4C54c5ccccc5-c5ccccc54)cc3)cc2)cc1 ; c1ccc(-c2ccc(N(c3ccc(-c4ccccc4)cc3)c3ccc4c(c3)-c3ccccc3C43c4ccccc4-c4ccccc43)cc2)cc1 ; CC1(C)c2ccccc2-c2ccc(N(c3ccc(-c4ccccc4)cc3)c3cccc4c3-c3ccccc3C43c4ccccc4-c4ccccc43)cc21]

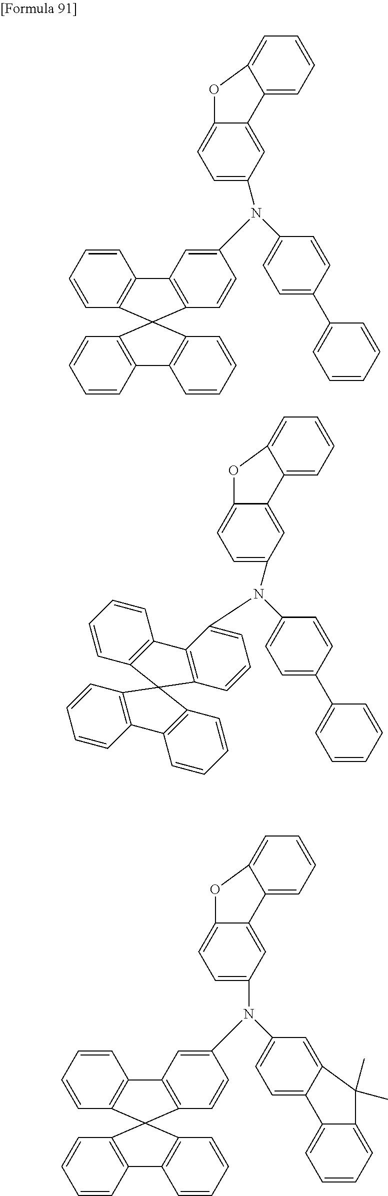

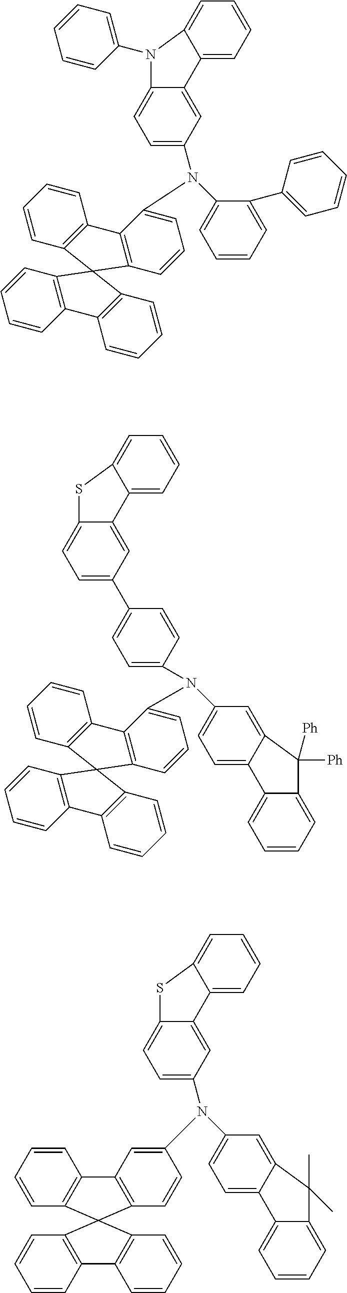 [SMILES: c1ccc(-c2ccccc2N(c2ccc3c(c2)c2ccccc2n3-c2ccccc2)c2cccc3c2-c2ccccc2C32c3ccccc3-c3ccccc32)cc1 ; c1ccc(C2(c3ccccc3)c3ccccc3-c3ccc(N(c4ccc(-c5ccc6sc7ccccc7c6c5)cc4)c4cccc5c4-c4ccccc4C54c5ccccc5-c5ccccc54)cc32)cc1 ; CC1(C)c2ccccc2-c2ccc(N(c3ccc4c(c3)-c3ccccc3C43c4ccccc4-c4ccccc43)c3ccc4sc5ccccc5c4c3)cc21]

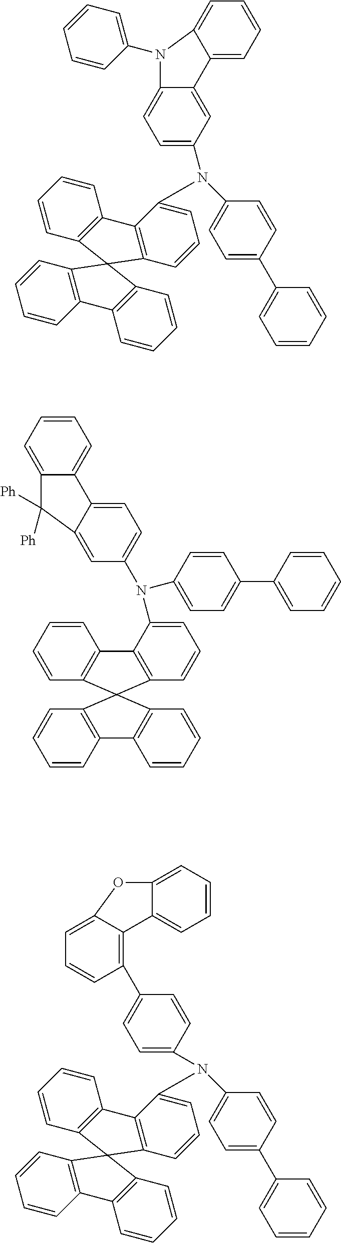 [SMILES: c1ccc(-c2ccc(N(c3ccc4c(c3)c3ccccc3n4-c3ccccc3)c3cccc4c3-c3ccccc3C43c4ccccc4-c4ccccc43)cc2)cc1 ; c1ccc(-c2ccc(N(c3ccc4c(c3)C(c3ccccc3)(c3ccccc3)c3ccccc3-4)c3cccc4c3-c3ccccc3C43c4ccccc4-c4ccccc43)cc2)cc1 ; c1ccc(-c2ccc(N(c3ccc(-c4cccc5oc6ccccc6c45)cc3)c3cccc4c3-c3ccccc3C43c4ccccc4-c4ccccc43)cc2)cc1]

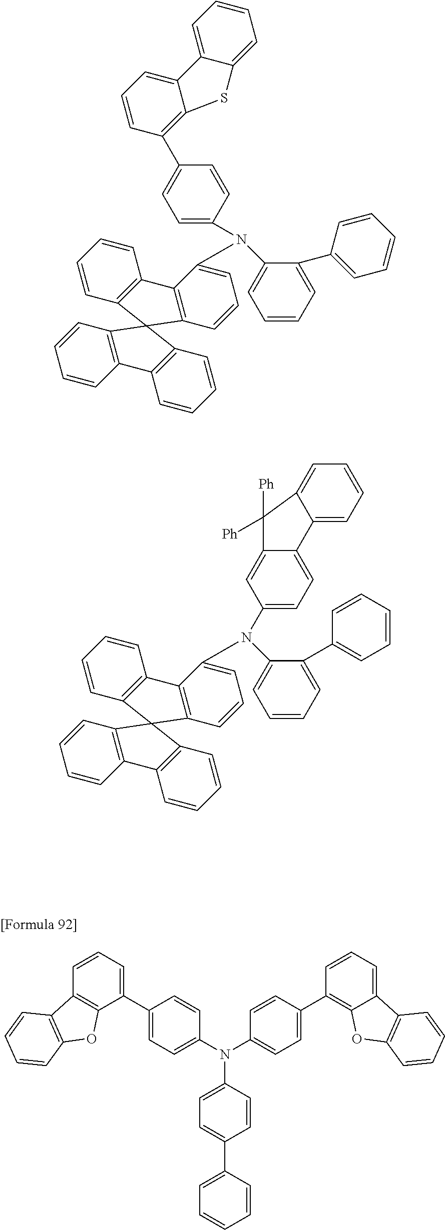 [SMILES: c1ccc(-c2ccccc2N(c2ccc(-c3cccc4c3sc3ccccc34)cc2)c2cccc3c2-c2ccccc2C32c3ccccc3-c3ccccc32)cc1 ; c1ccc(-c2ccccc2N(c2ccc3c(c2)C(c2ccccc2)(c2ccccc2)c2ccccc2-3)c2cccc3c2-c2ccccc2C32c3ccccc3-c3ccccc32)cc1 ; c1ccc(-c2ccc(N(c3ccc(-c4cccc5c4oc4ccccc45)cc3)c3ccc(-c4cccc5c4oc4ccccc45)cc3)cc2)cc1]

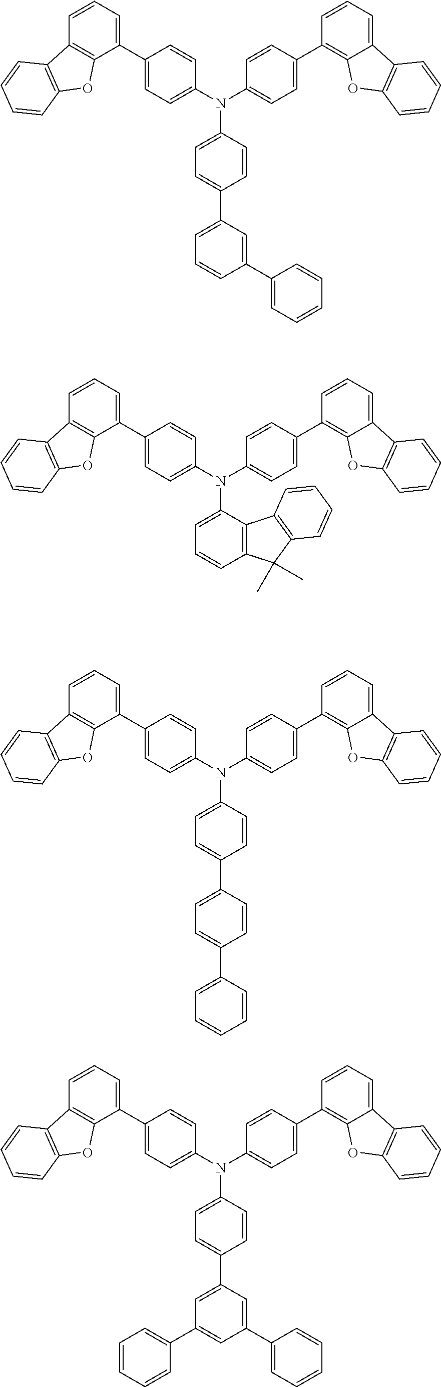 [SMILES: c1ccc(-c2cccc(-c3ccc(N(c4ccc(-c5cccc6c5oc5ccccc56)cc4)c4ccc(-c5cccc6c5oc5ccccc56)cc4)cc3)c2)cc1 ; CC1(C)c2ccccc2-c2c(N(c3ccc(-c4cccc5c4oc4ccccc45)cc3)c3ccc(-c4cccc5c4oc4ccccc45)cc3)cccc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

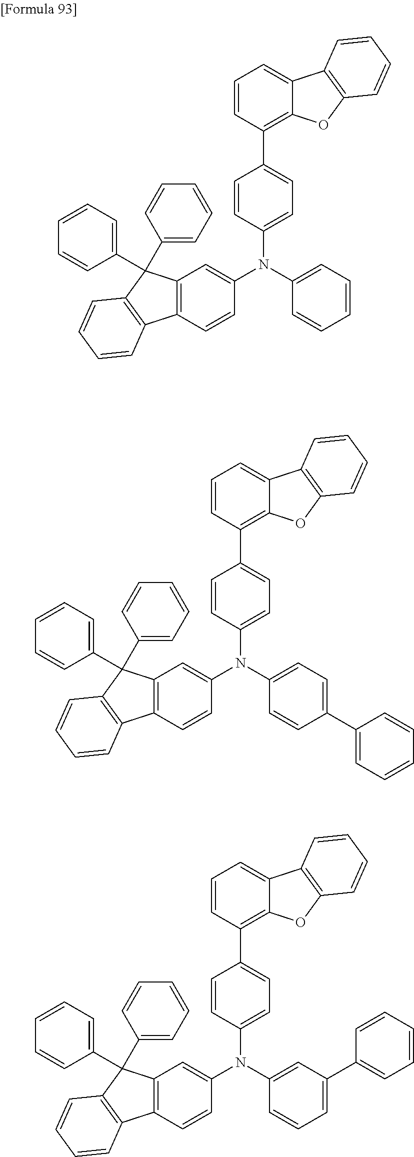 [SMILES: c1ccc(N(c2ccc(-c3cccc4c3oc3ccccc34)cc2)c2ccc3c(c2)C(c2ccccc2)(c2ccccc2)c2ccccc2-3)cc1 ; c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; c1ccc(-c2cccc(N(c3ccc(-c4cccc5c4oc4ccccc45)cc3)c3ccc4c(c3)C(c3ccccc3)(c3ccccc3)c3ccccc3-4)c2)cc1]

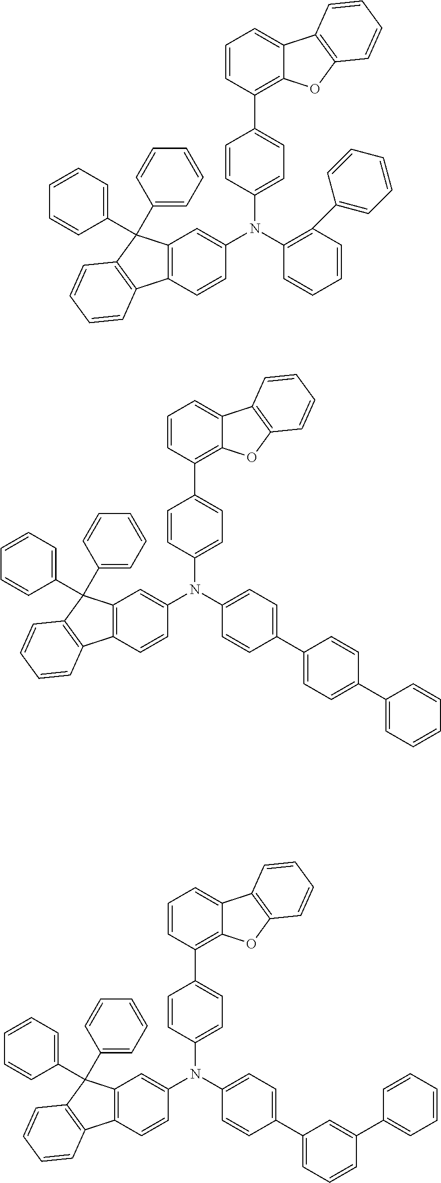 [SMILES: c1ccc(-c2ccccc2N(c2ccc(-c3cccc4c3oc3ccccc34)cc2)c2ccc3c(c2)C(c2ccccc2)(c2ccccc2)c2ccccc2-3)cc1 ; c1ccc(-c2ccc(-c3ccc(N(c4ccc(-c5cccc6c5oc5ccccc56)cc4)c4ccc5c(c4)C(c4ccccc4)(c4ccccc4)c4ccccc4-5)cc3)cc2)cc1 ; c1ccc(-c2cccc(-c3ccc(N(c4ccc(-c5cccc6c5oc5ccccc56)cc4)c4ccc5c(c4)C(c4ccccc4)(c4ccccc4)c4ccccc4-5)cc3)c2)cc1]

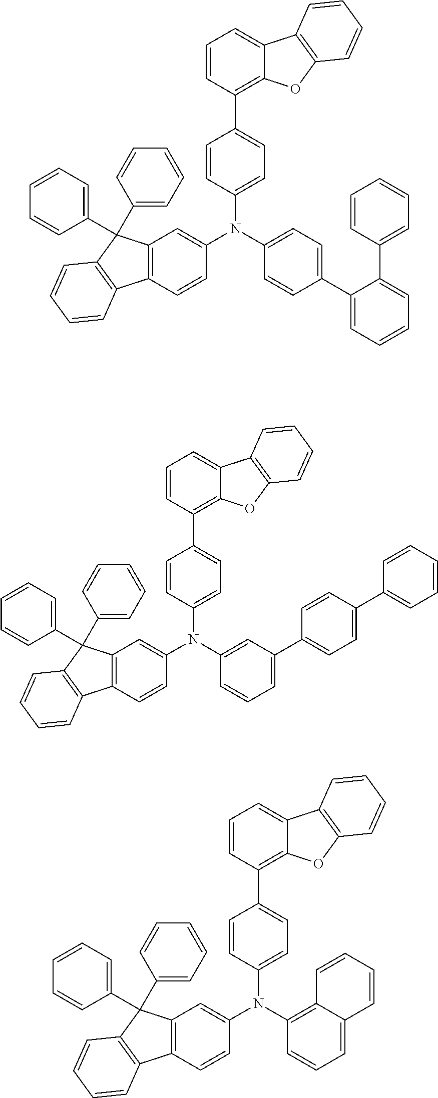 [SMILES: c1ccc(-c2ccccc2-c2ccc(N(c3ccc(-c4cccc5c4oc4ccccc45)cc3)c3ccc4c(c3)C(c3ccccc3)(c3ccccc3)c3ccccc3-4)cc2)cc1 ; c1ccc(-c2ccc(-c3cccc(N(c4ccc(-c5cccc6c5oc5ccccc56)cc4)c4ccc5c(c4)C(c4ccccc4)(c4ccccc4)c4ccccc4-5)c3)cc2)cc1 ; c1ccc(C2(c3ccccc3)c3ccccc3-c3ccc(N(c4ccc(-c5cccc6c5oc5ccccc56)cc4)c4cccc5ccccc45)cc32)cc1]

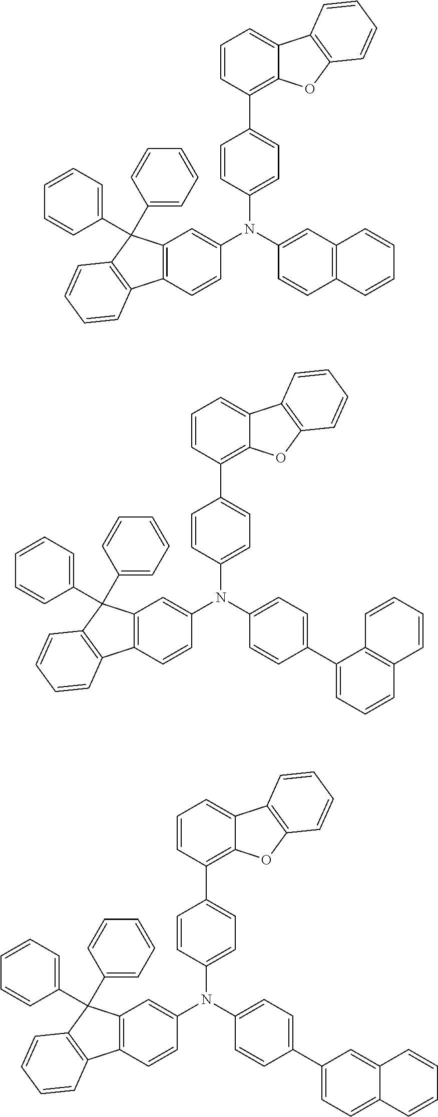 [SMILES: c1ccc(C2(c3ccccc3)c3ccccc3-c3ccc(N(c4ccc(-c5cccc6c5oc5ccccc56)cc4)c4ccc5ccccc5c4)cc32)cc1 ; c1ccc(C2(c3ccccc3)c3ccccc3-c3ccc(N(c4ccc(-c5cccc6ccccc56)cc4)c4ccc(-c5cccc6c5oc5ccccc56)cc4)cc32)cc1 ; c1ccc(C2(c3ccccc3)c3ccccc3-c3ccc(N(c4ccc(-c5ccc6ccccc6c5)cc4)c4ccc(-c5cccc6c5oc5ccccc56)cc4)cc32)cc1]

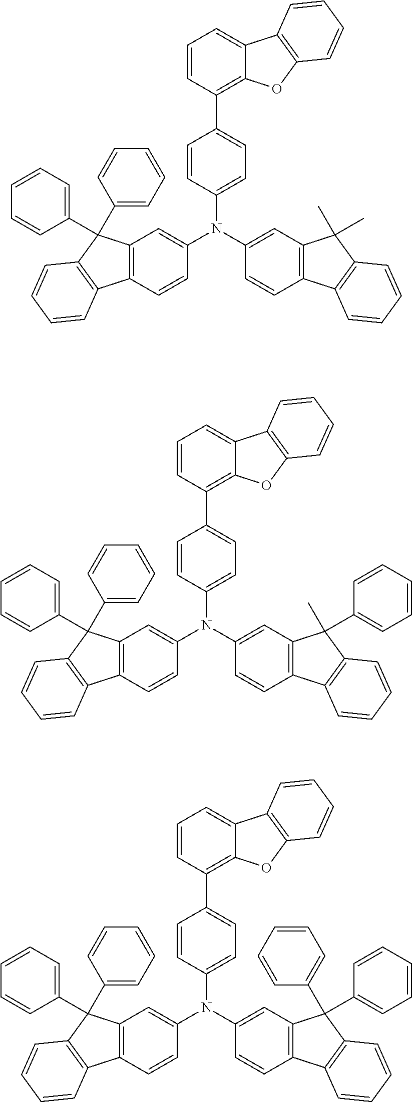 [SMILES: CC1(C)c2ccccc2-c2ccc(N(c3ccc(-c4cccc5c4oc4ccccc45)cc3)c3ccc4c(c3)C(c3ccccc3)(c3ccccc3)c3ccccc3-4)cc21 ; CC1(c2ccccc2)c2ccccc2-c2ccc(N(c3ccc(-c4cccc5c4oc4ccccc45)cc3)c3ccc4c(c3)C(c3ccccc3)(c3ccccc3)c3ccccc3-4)cc21 ; c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1]

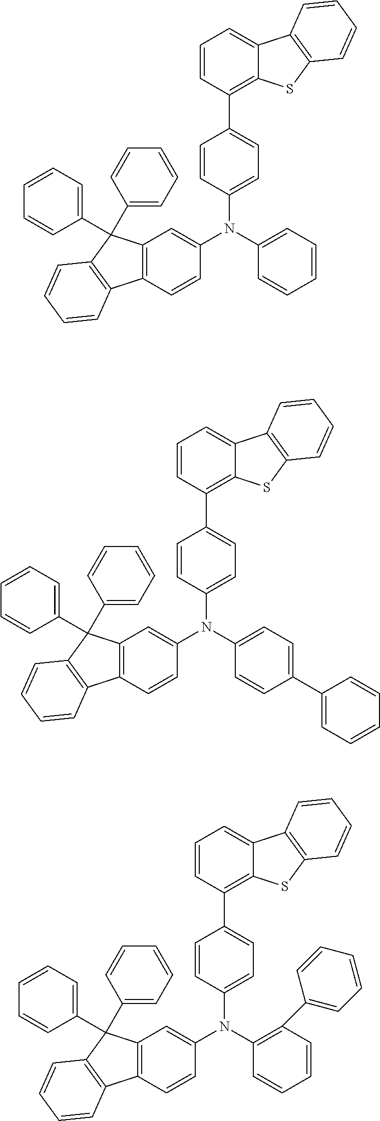 [SMILES: c1ccc(N(c2ccc(-c3cccc4c3sc3ccccc34)cc2)c2ccc3c(c2)C(c2ccccc2)(c2ccccc2)c2ccccc2-3)cc1 ; c1ccc(-c2ccc(N(c3ccc(-c4cccc5c4sc4ccccc45)cc3)c3ccc4c(c3)C(c3ccccc3)(c3ccccc3)c3ccccc3-4)cc2)cc1 ; c1ccc(-c2ccccc2N(c2ccc(-c3cccc4c3sc3ccccc34)cc2)c2ccc3c(c2)C(c2ccccc2)(c2ccccc2)c2ccccc2-3)cc1]

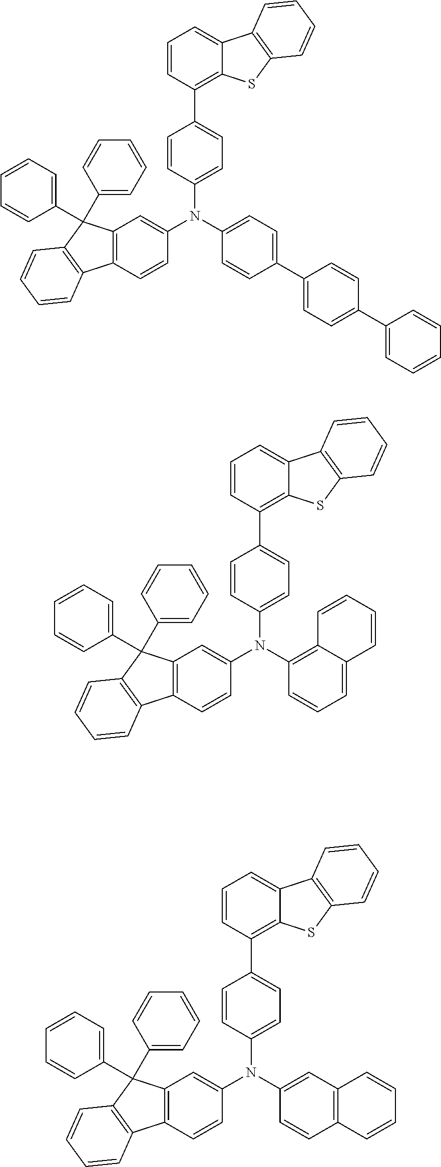 [SMILES: c1ccc(-c2ccc(-c3ccc(N(c4ccc(-c5cccc6c5sc5ccccc56)cc4)c4ccc5c(c4)C(c4ccccc4)(c4ccccc4)c4ccccc4-5)cc3)cc2)cc1 ; c1ccc(C2(c3ccccc3)c3ccccc3-c3ccc(N(c4ccc(-c5cccc6c5sc5ccccc56)cc4)c4cccc5ccccc45)cc32)cc1 ; c1ccc(C2(c3ccccc3)c3ccccc3-c3ccc(N(c4ccc(-c5cccc6c5sc5ccccc56)cc4)c4ccc5ccccc5c4)cc32)cc1]

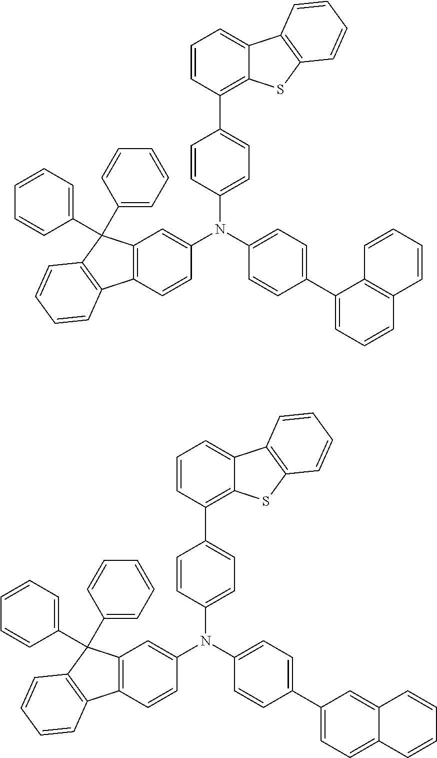 [SMILES: c1ccc(C2(c3ccccc3)c3ccccc3-c3ccc(N(c4ccc(-c5cccc6ccccc56)cc4)c4ccc(-c5cccc6c5sc5ccccc56)cc4)cc32)cc1 ; c1ccc(C2(c3ccccc3)c3ccccc3-c3ccc(N(c4ccc(-c5ccc6ccccc6c5)cc4)c4ccc(-c5cccc6c5sc5ccccc56)cc4)cc32)cc1]

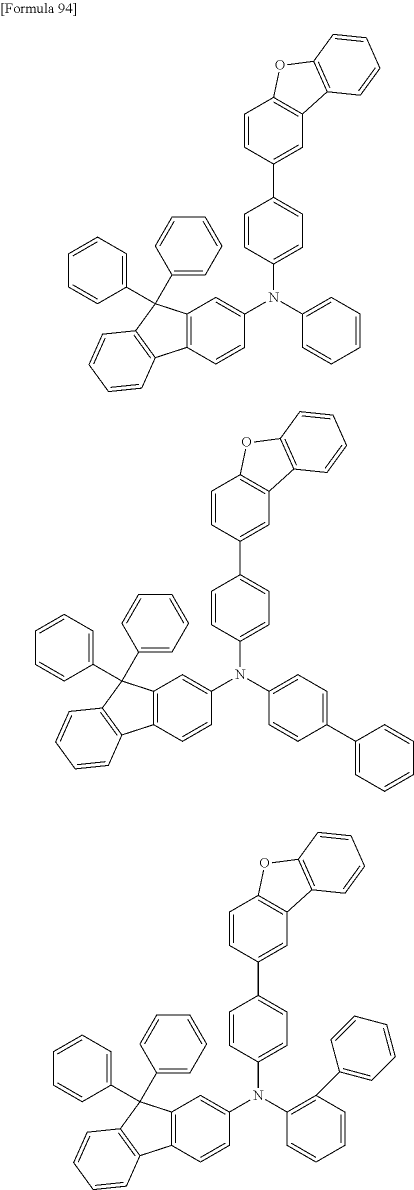 [SMILES: c1ccc(N(c2ccc(-c3ccc4oc5ccccc5c4c3)cc2)c2ccc3c(c2)C(c2ccccc2)(c2ccccc2)c2ccccc2-3)cc1 ; c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccccc2N(c2ccc(-c3ccc4oc5ccccc5c4c3)cc2)c2ccc3c(c2)C(c2ccccc2)(c2ccccc2)c2ccccc2-3)cc1]

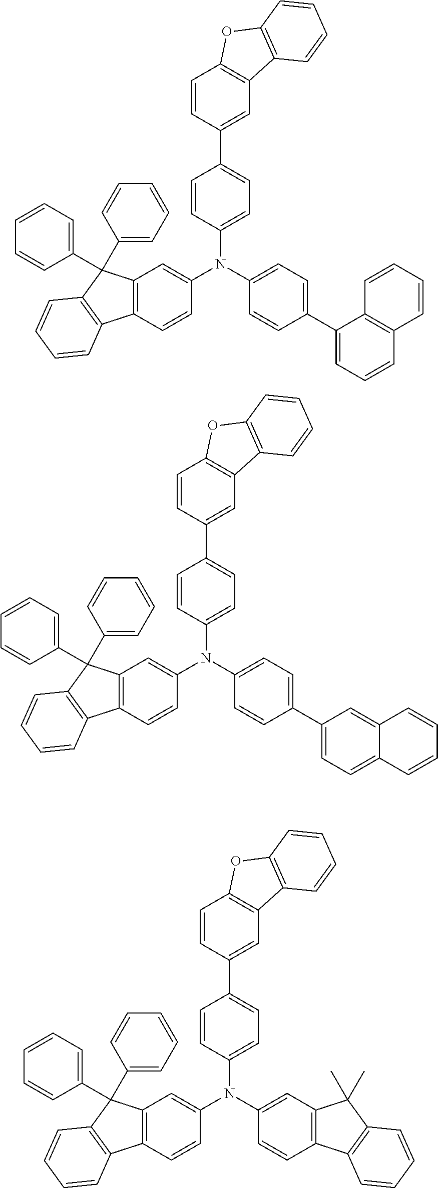 [SMILES: c1ccc(C2(c3ccccc3)c3ccccc3-c3ccc(N(c4ccc(-c5ccc6oc7ccccc7c6c5)cc4)c4ccc(-c5cccc6ccccc56)cc4)cc32)cc1 ; c1ccc(C2(c3ccccc3)c3ccccc3-c3ccc(N(c4ccc(-c5ccc6ccccc6c5)cc4)c4ccc(-c5ccc6oc7ccccc7c6c5)cc4)cc32)cc1 ; CC1(C)c2ccccc2-c2ccc(N(c3ccc(-c4ccc5oc6ccccc6c5c4)cc3)c3ccc4c(c3)C(c3ccccc3)(c3ccccc3)c3ccccc3-4)cc21]

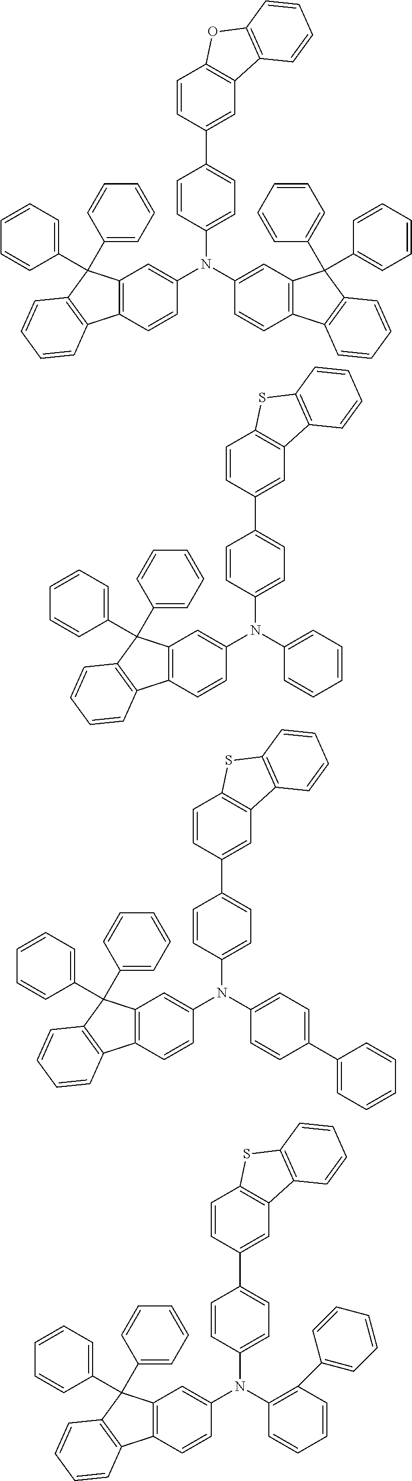

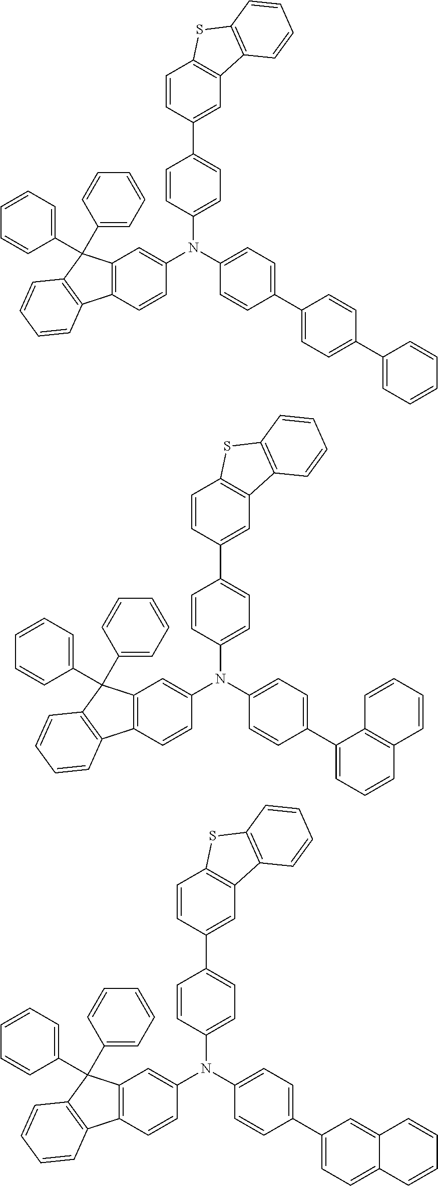 [SMILES: c1ccc(-c2ccc(-c3ccc(N(c4ccc(-c5ccc6sc7ccccc7c6c5)cc4)c4ccc5c(c4)C(c4ccccc4)(c4ccccc4)c4ccccc4-5)cc3)cc2)cc1 ; c1ccc(C2(c3ccccc3)c3ccccc3-c3ccc(N(c4ccc(-c5ccc6sc7ccccc7c6c5)cc4)c4ccc(-c5cccc6ccccc56)cc4)cc32)cc1 ; c1ccc(C2(c3ccccc3)c3ccccc3-c3ccc(N(c4ccc(-c5ccc6ccccc6c5)cc4)c4ccc(-c5ccc6sc7ccccc7c6c5)cc4)cc32)cc1]

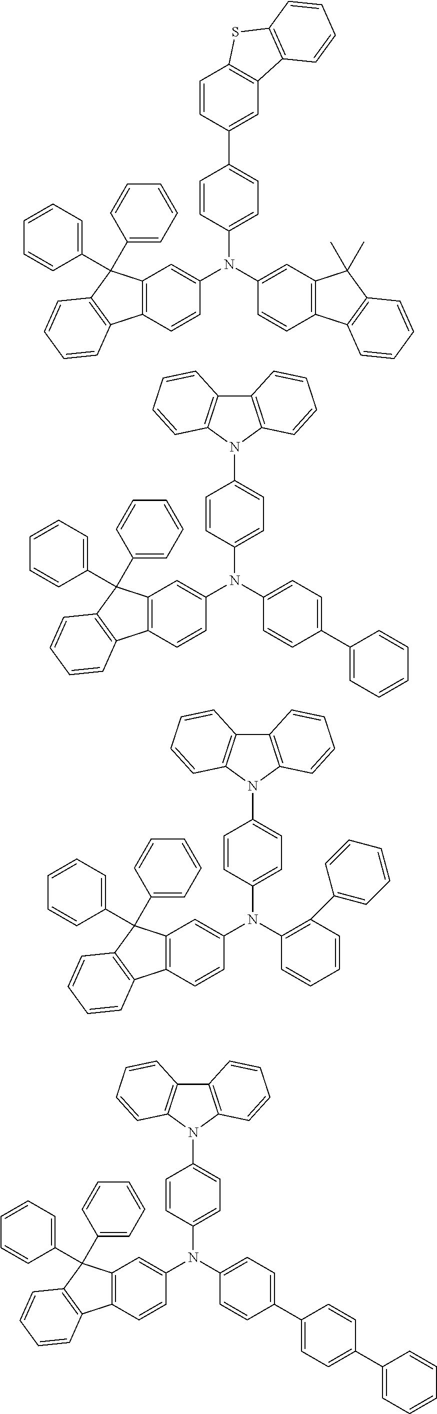

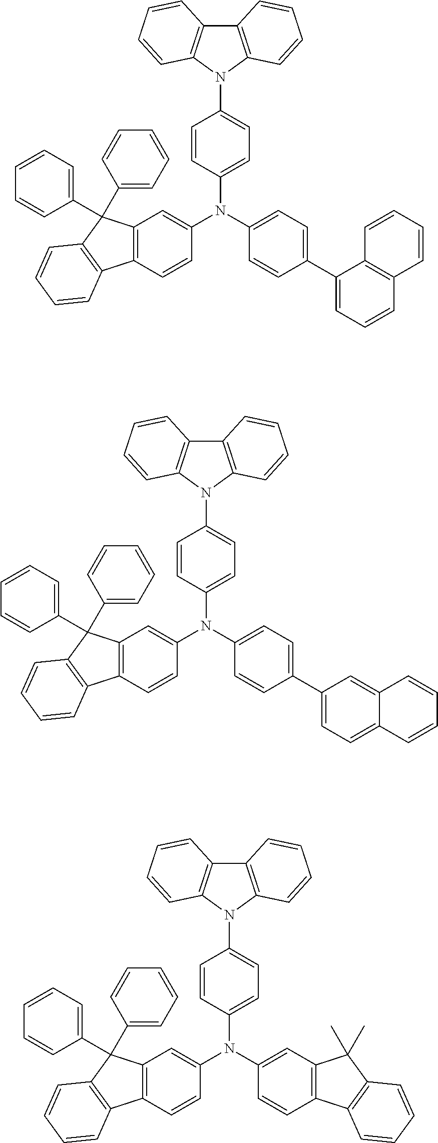 [SMILES: c1ccc(C2(c3ccccc3)c3ccccc3-c3ccc(N(c4ccc(-c5cccc6ccccc56)cc4)c4ccc(-n5c6ccccc6c6ccccc65)cc4)cc32)cc1 ; c1ccc(C2(c3ccccc3)c3ccccc3-c3ccc(N(c4ccc(-c5ccc6ccccc6c5)cc4)c4ccc(-n5c6ccccc6c6ccccc65)cc4)cc32)cc1 ; CC1(C)c2ccccc2-c2ccc(N(c3ccc(-n4c5ccccc5c5ccccc54)cc3)c3ccc4c(c3)C(c3ccccc3)(c3ccccc3)c3ccccc3-4)cc21]

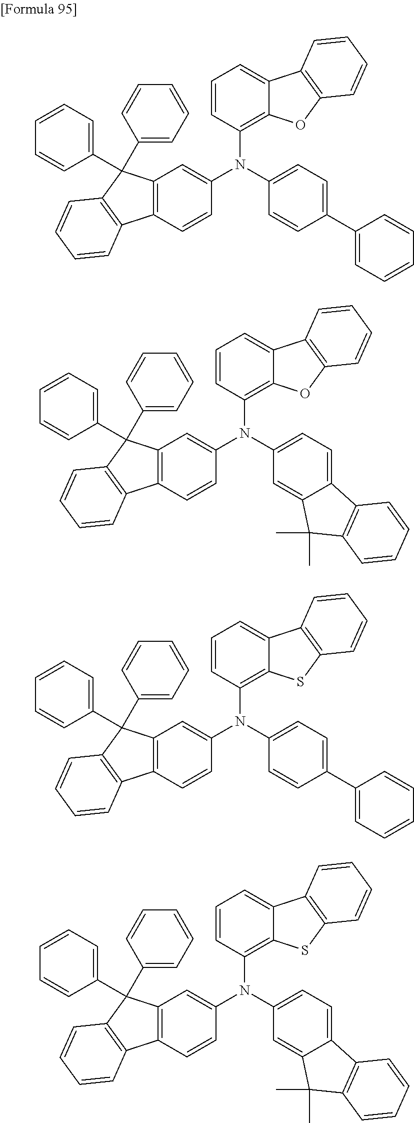

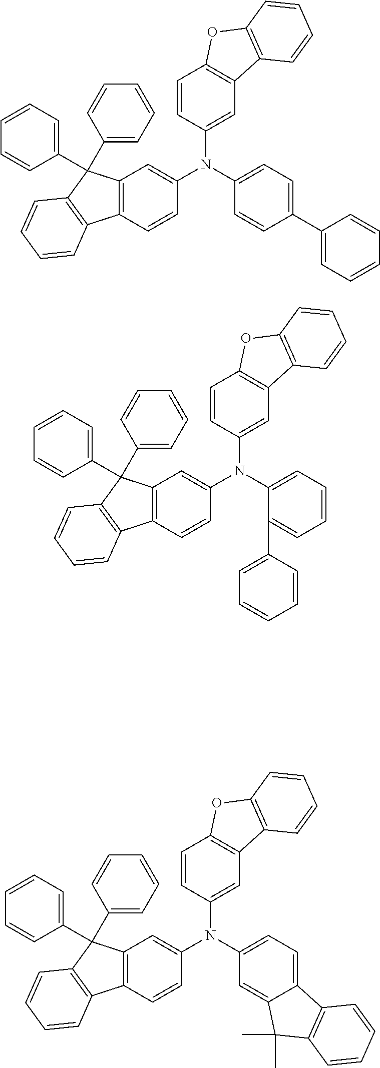

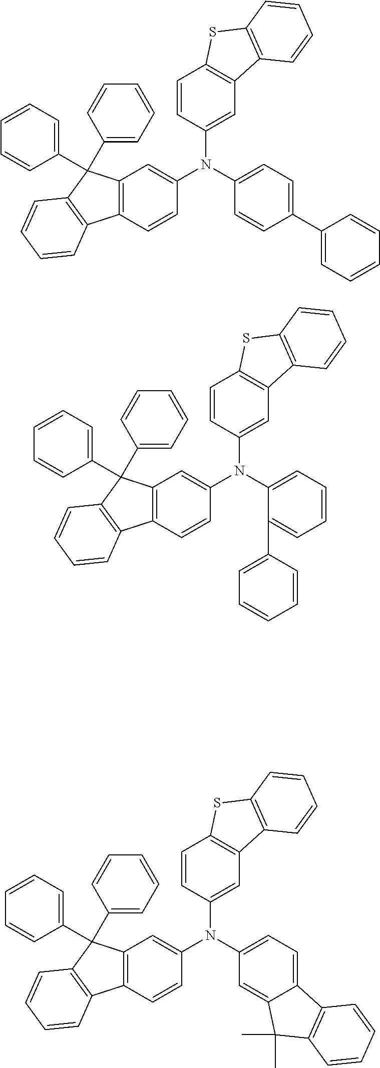

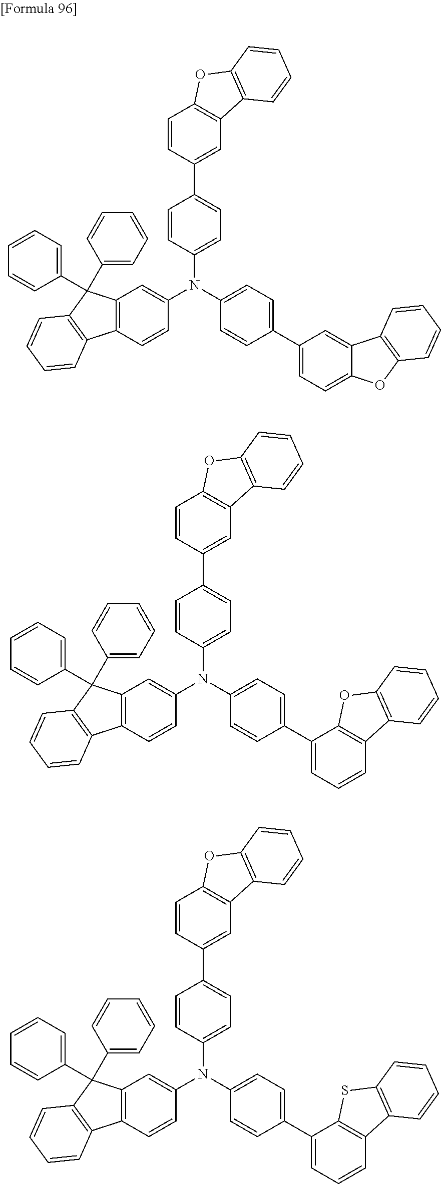 [SMILES: c1ccc(C2(c3ccccc3)c3ccccc3-c3ccc(N(c4ccc(-c5ccc6oc7ccccc7c6c5)cc4)c4ccc(-c5ccc6oc7ccccc7c6c5)cc4)cc32)cc1 ; c1ccc(C2(c3ccccc3)c3ccccc3-c3ccc(N(c4ccc(-c5ccc6oc7ccccc7c6c5)cc4)c4ccc(-c5cccc6c5oc5ccccc56)cc4)cc32)cc1 ; c1ccc(C2(c3ccccc3)c3ccccc3-c3ccc(N(c4ccc(-c5ccc6oc7ccccc7c6c5)cc4)c4ccc(-c5cccc6c5sc5ccccc56)cc4)cc32)cc1]

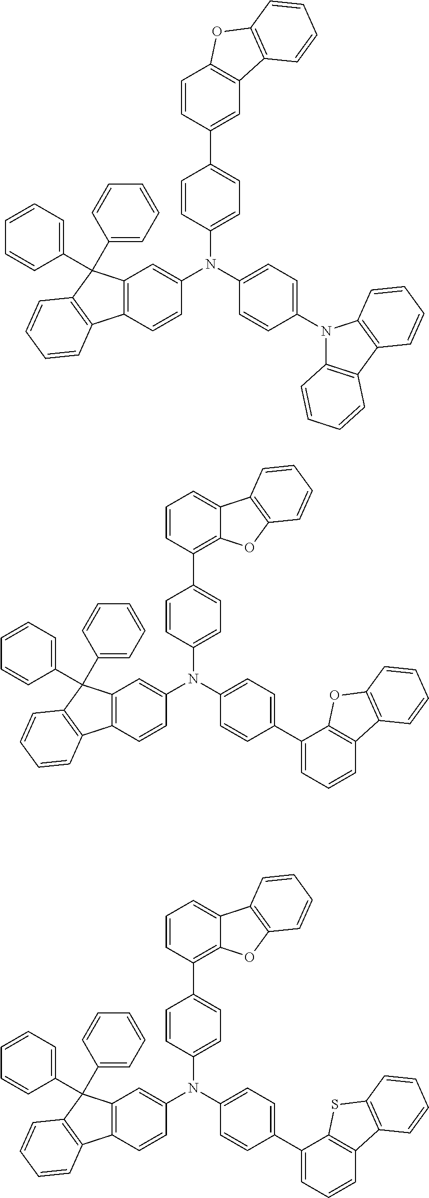 [SMILES: c1ccc(C2(c3ccccc3)c3ccccc3-c3ccc(N(c4ccc(-c5ccc6oc7ccccc7c6c5)cc4)c4ccc(-n5c6ccccc6c6ccccc65)cc4)cc32)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(C2(c3ccccc3)c3ccccc3-c3ccc(N(c4ccc(-c5cccc6c5oc5ccccc56)cc4)c4ccc(-c5cccc6c5sc5ccccc56)cc4)cc32)cc1]

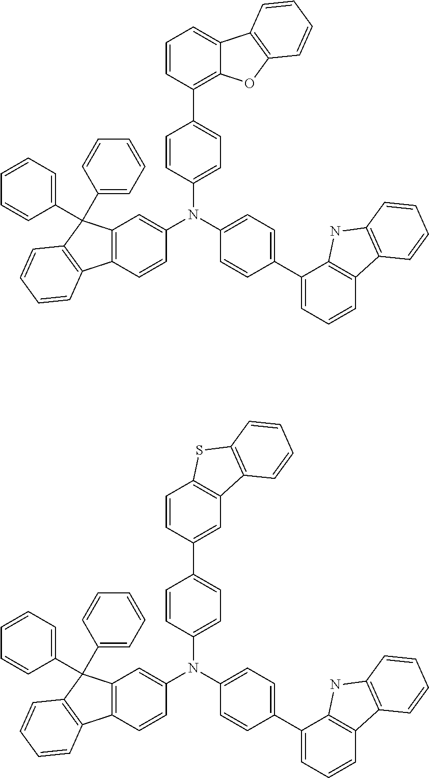 [SMILES: c1ccc(C2(c3ccccc3)c3ccccc3-c3ccc(N(c4ccc(-c5cccc6c5[nH]c5ccccc56)cc4)c4ccc(-c5cccc6c5oc5ccccc56)cc4)cc32)cc1 ; c1ccc(C2(c3ccccc3)c3ccccc3-c3ccc(N(c4ccc(-c5ccc6sc7ccccc7c6c5)cc4)c4ccc(-c5cccc6c5[nH]c5ccccc56)cc4)cc32)cc1]

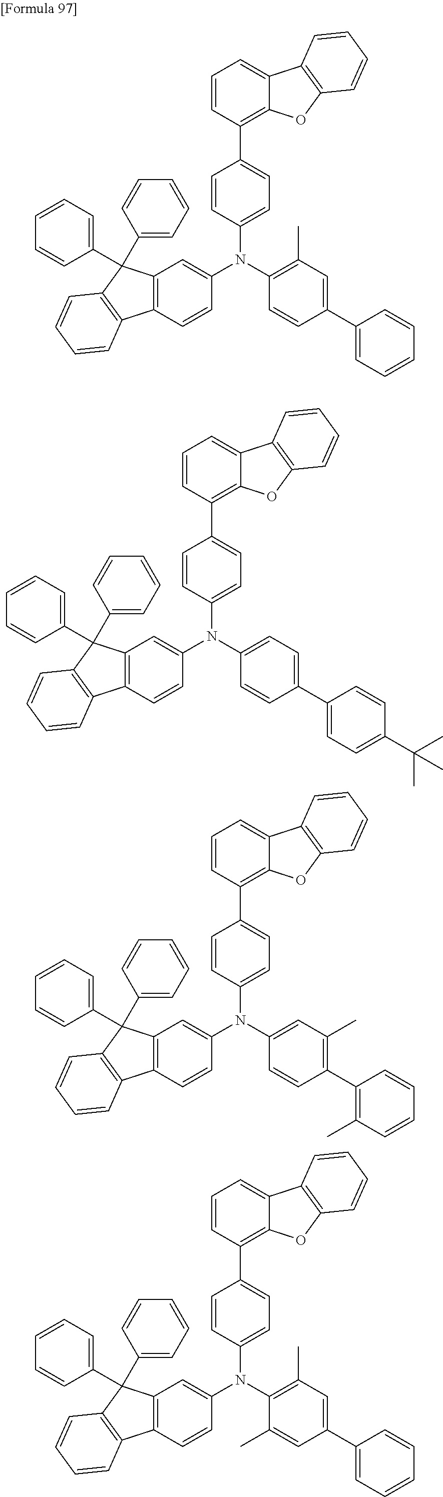

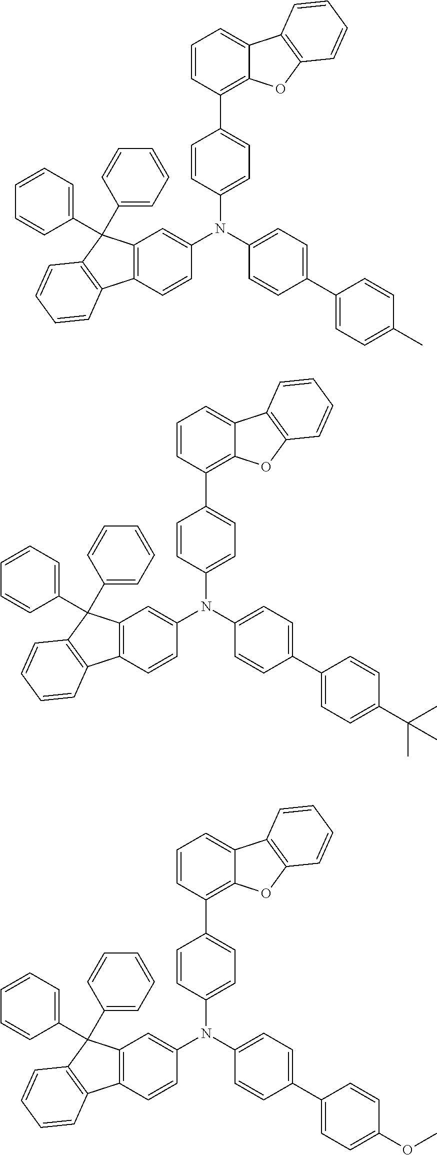 [SMILES: Cc1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; CC(C)(C)c1ccc(-c2ccc(N(c3ccc(-c4cccc5c4oc4ccccc45)cc3)c3ccc4c(c3)C(c3ccccc3)(c3ccccc3)c3ccccc3-4)cc2)cc1 ; COc1ccc(-c2ccc(N(c3ccc(-c4cccc5c4oc4ccccc45)cc3)c3ccc4c(c3)C(c3ccccc3)(c3ccccc3)c3ccccc3-4)cc2)cc1]

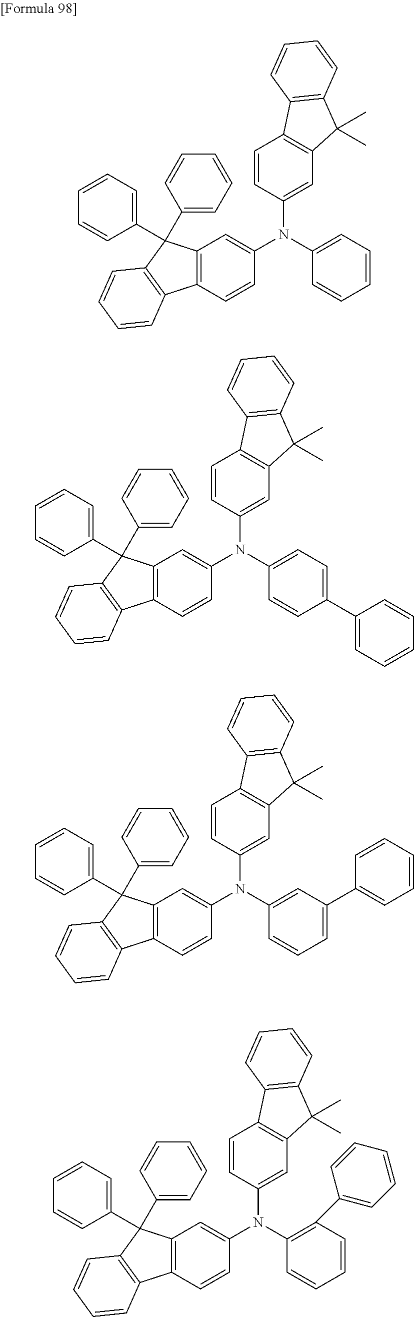 [SMILES: CC1(C)c2ccccc2-c2ccc(N(c3ccccc3)c3ccc4c(c3)C(c3ccccc3)(c3ccccc3)c3ccccc3-4)cc21 ; CC1(C)c2ccccc2-c2ccc(N(c3ccc(-c4ccccc4)cc3)c3ccc4c(c3)C(c3ccccc3)(c3ccccc3)c3ccccc3-4)cc21 ; CC1(C)c2ccccc2-c2ccc(N(c3cccc(-c4ccccc4)c3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccccc3-c3ccccc3)cc21]

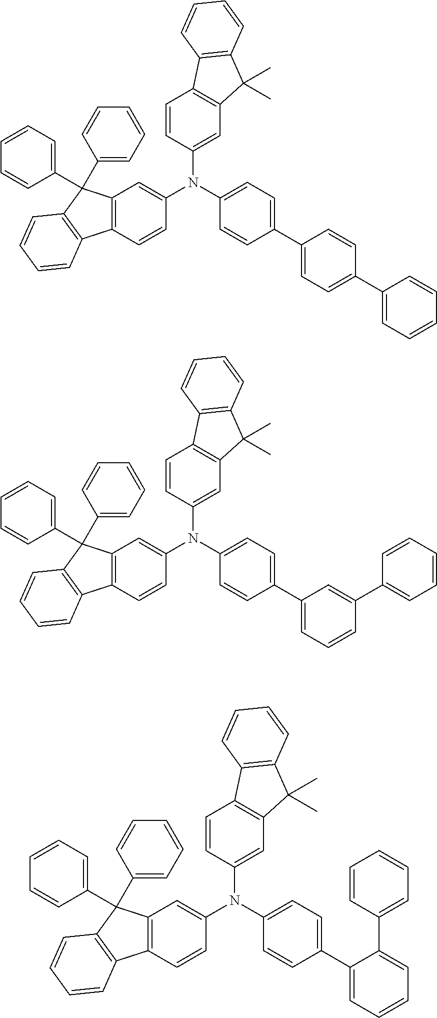 [SMILES: CC1(C)c2ccccc2-c2ccc(N(c3ccc(-c4ccc(-c5ccccc5)cc4)cc3)c3ccc4c(c3)C(c3ccccc3)(c3ccccc3)c3ccccc3-4)cc21 ; CC1(C)c2ccccc2-c2ccc(N(c3ccc(-c4cccc(-c5ccccc5)c4)cc3)c3ccc4c(c3)C(c3ccccc3)(c3ccccc3)c3ccccc3-4)cc21 ; CC1(C)c2ccccc2-c2ccc(N(c3ccc(-c4ccccc4-c4ccccc4)cc3)c3ccc4c(c3)C(c3ccccc3)(c3ccccc3)c3ccccc3-4)cc21]

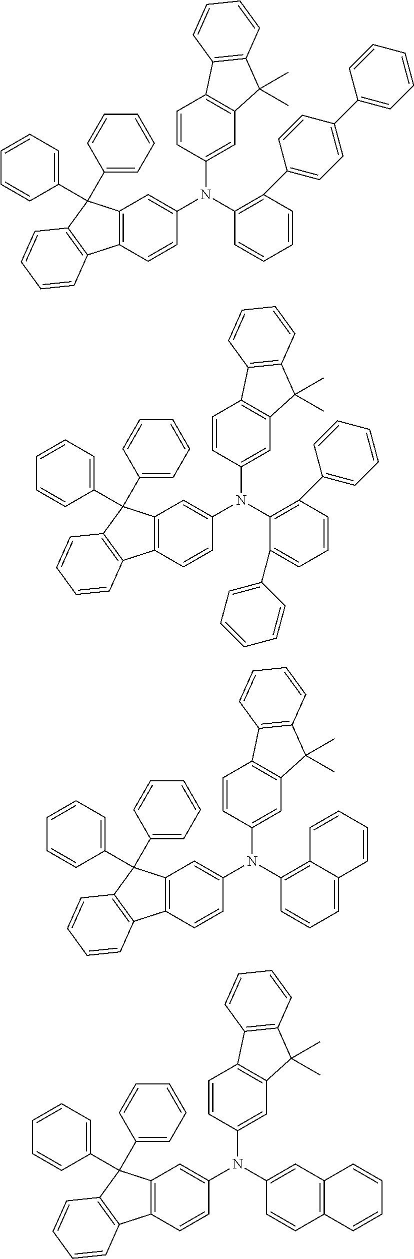

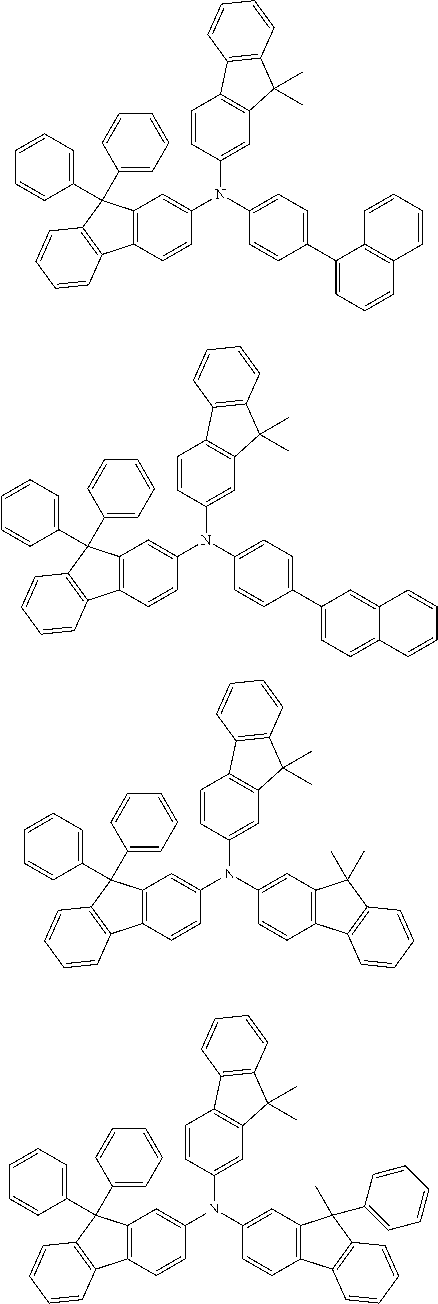 [SMILES: CC1(C)c2ccccc2-c2ccc(N(c3ccc(-c4cccc5ccccc45)cc3)c3ccc4c(c3)C(c3ccccc3)(c3ccccc3)c3ccccc3-4)cc21 ; CC1(C)c2ccccc2-c2ccc(N(c3ccc(-c4ccc5ccccc5c4)cc3)c3ccc4c(c3)C(c3ccccc3)(c3ccccc3)c3ccccc3-4)cc21 ; CC1(C)c2ccccc2-c2ccc(N(c3ccc4c(c3)C(C)(C)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C(C)(c3ccccc3)c3ccccc3-4)c3ccc4c(c3)C(c3ccccc3)(c3ccccc3)c3ccccc3-4)cc21]

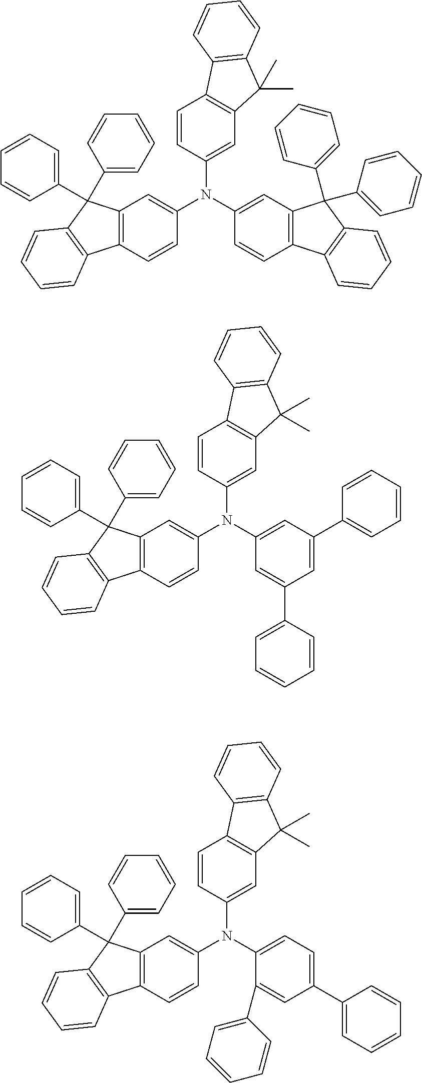 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3cc(-c4ccccc4)cc(-c4ccccc4)c3)c3ccc4c(c3)C(c3ccccc3)(c3ccccc3)c3ccccc3-4)cc21 ; CC1(C)c2ccccc2-c2ccc(N(c3ccc4c(c3)C(c3ccccc3)(c3ccccc3)c3ccccc3-4)c3ccc(-c4ccccc4)cc3-c3ccccc3)cc21]

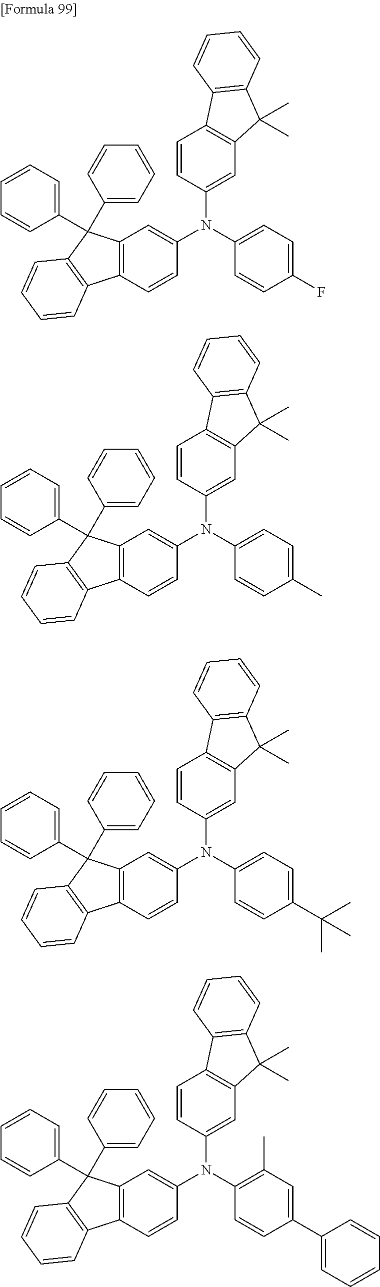 [SMILES: CC1(C)c2ccccc2-c2ccc(N(c3ccc(F)cc3)c3ccc4c(c3)C(c3ccccc3)(c3ccccc3)c3ccccc3-4)cc21 ; Cc1ccc(N(c2ccc3c(c2)C(C)(C)c2ccccc2-3)c2ccc3c(c2)C(c2ccccc2)(c2ccccc2)c2ccccc2-3)cc1 ; CC(C)(C)c1ccc(N(c2ccc3c(c2)C(C)(C)c2ccccc2-3)c2ccc3c(c2)C(c2ccccc2)(c2ccccc2)c2ccccc2-3)cc1 ; Cc1cc(-c2ccccc2)ccc1N(c1ccc2c(c1)C(C)(C)c1ccccc1-2)c1ccc2c(c1)C(c1ccccc1)(c1ccccc1)c1ccccc1-2]

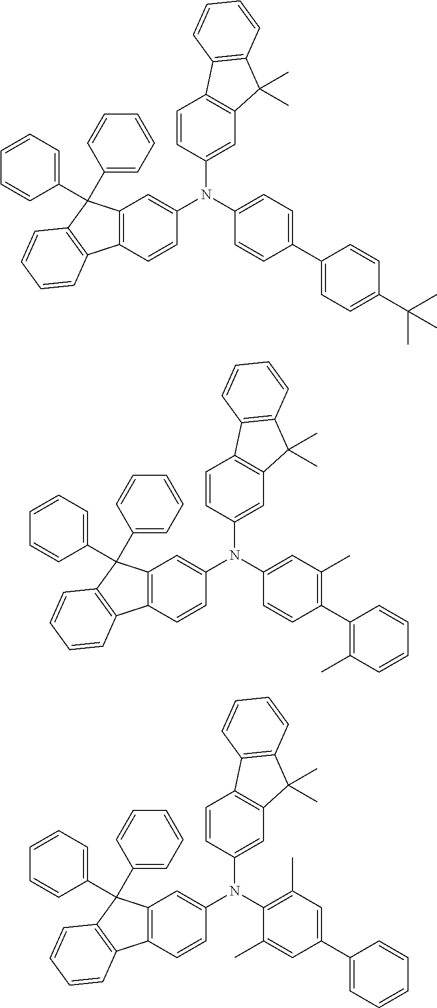 [SMILES: CC(C)(C)c1ccc(-c2ccc(N(c3ccc4c(c3)C(C)(C)c3ccccc3-4)c3ccc4c(c3)C(c3ccccc3)(c3ccccc3)c3ccccc3-4)cc2)cc1 ; Cc1ccccc1-c1ccc(N(c2ccc3c(c2)C(C)(C)c2ccccc2-3)c2ccc3c(c2)C(c2ccccc2)(c2ccccc2)c2ccccc2-3)cc1C ; Cc1cc(-c2ccccc2)cc(C)c1N(c1ccc2c(c1)C(C)(C)c1ccccc1-2)c1ccc2c(c1)C(c1ccccc1)(c1ccccc1)c1ccccc1-2]

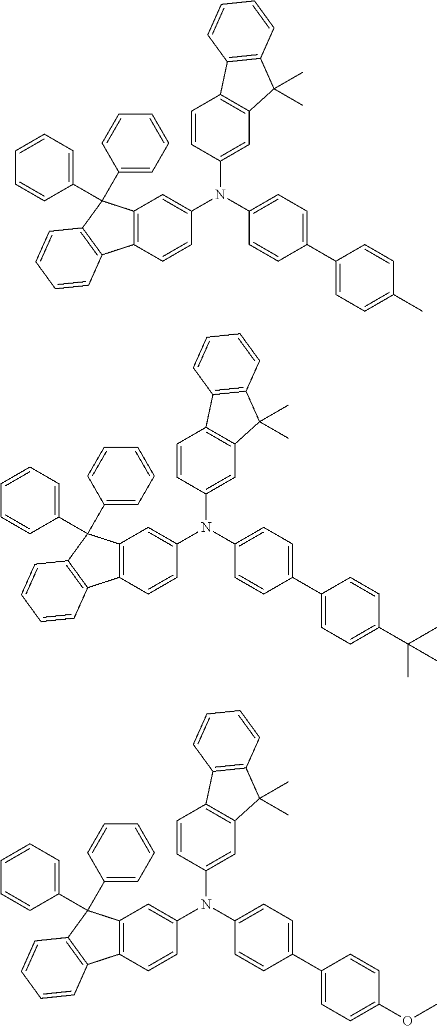 [SMILES: Cc1ccc(-c2ccc(N(c3ccc4c(c3)C(C)(C)c3ccccc3-4)c3ccc4c(c3)C(c3ccccc3)(c3ccccc3)c3ccccc3-4)cc2)cc1 ; CC(C)(C)c1ccc(-c2ccc(N(c3ccc4c(c3)C(C)(C)c3ccccc3-4)c3ccc4c(c3)C(c3ccccc3)(c3ccccc3)c3ccccc3-4)cc2)cc1 ; COc1ccc(-c2ccc(N(c3ccc4c(c3)C(C)(C)c3ccccc3-4)c3ccc4c(c3)C(c3ccccc3)(c3ccccc3)c3ccccc3-4)cc2)cc1]

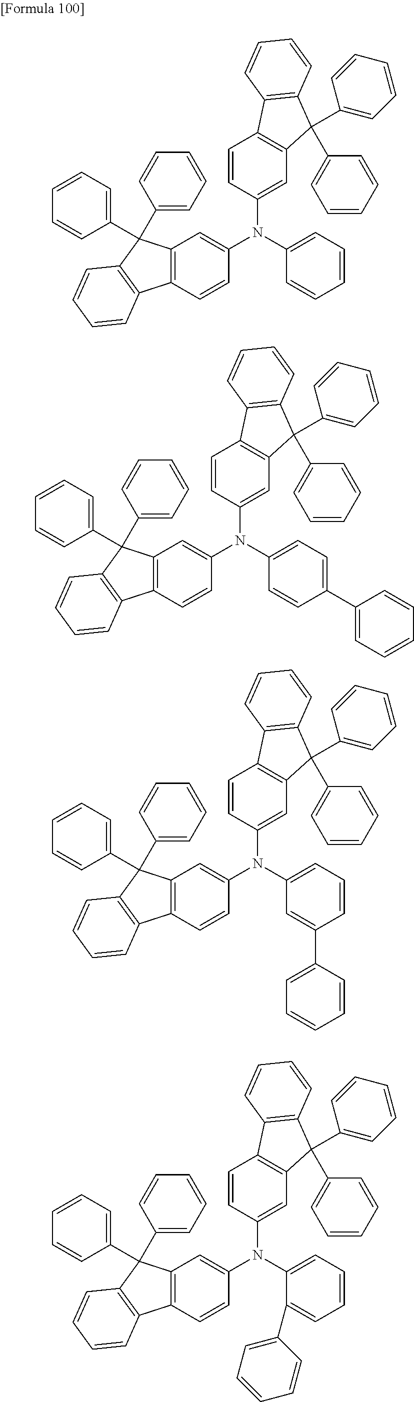

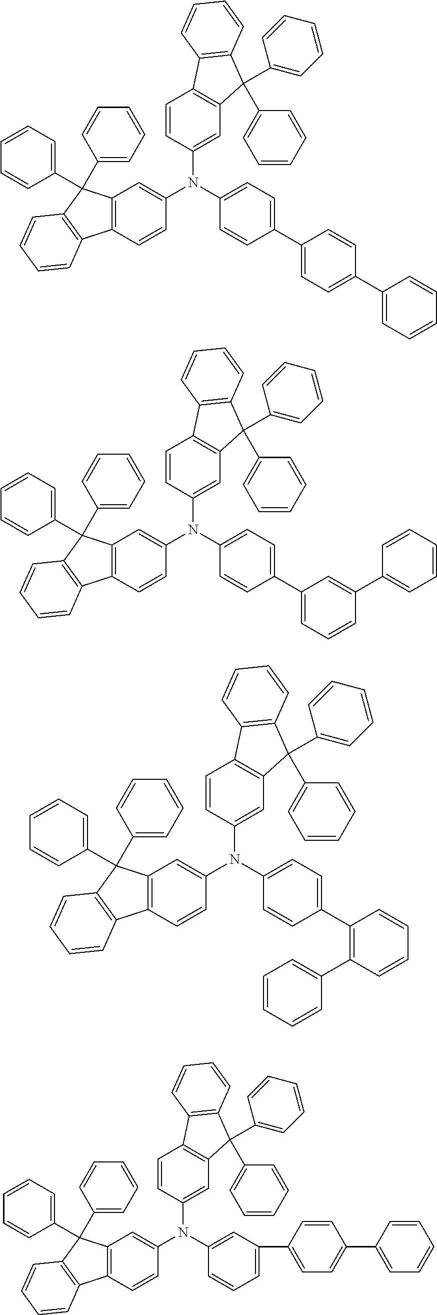

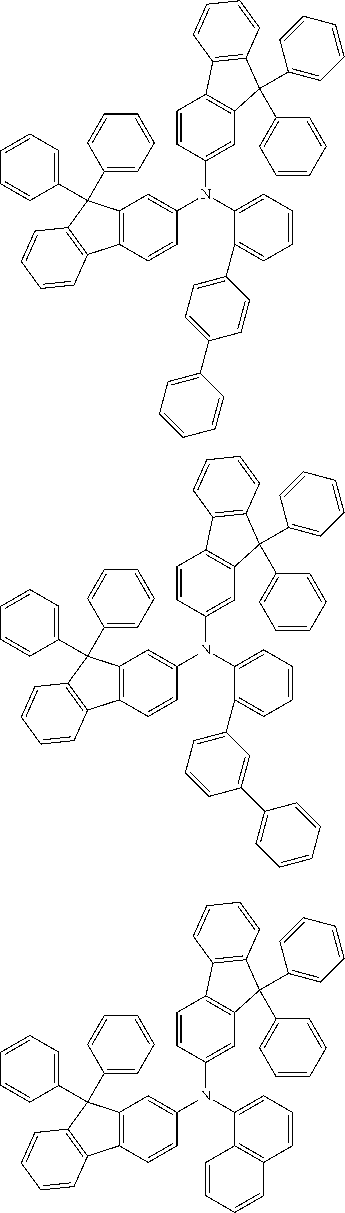

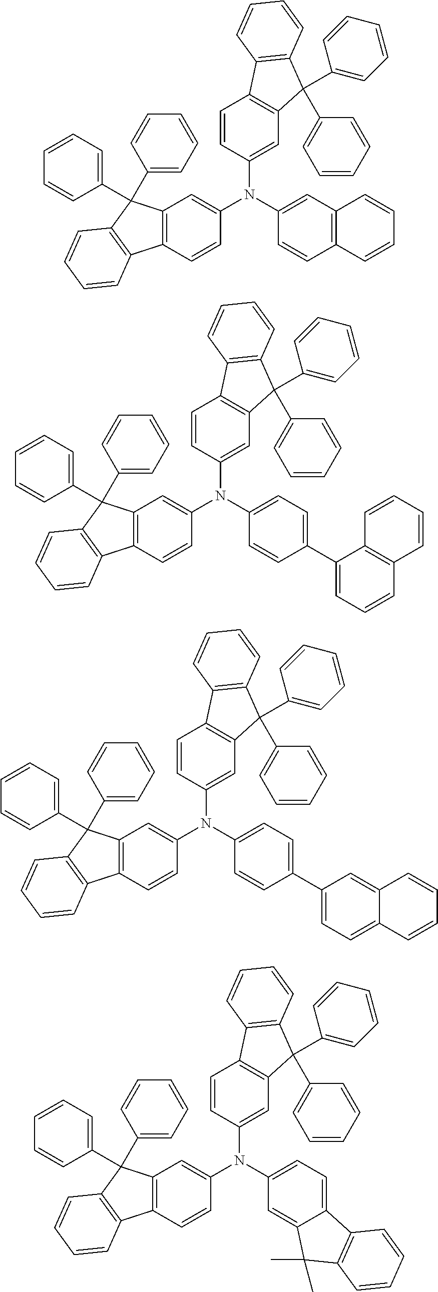

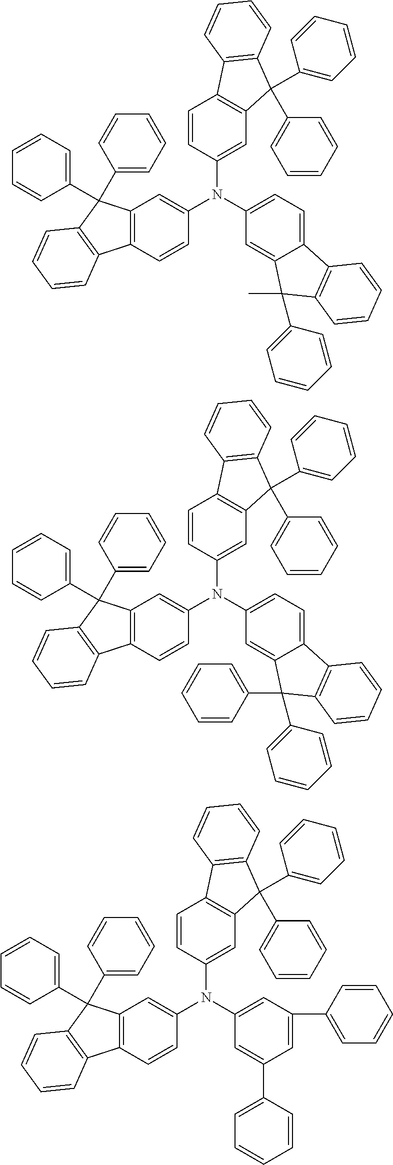

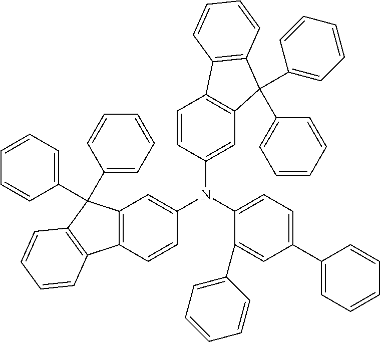 [SMILES: c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)c(-c3ccccc3)c2)cc1]

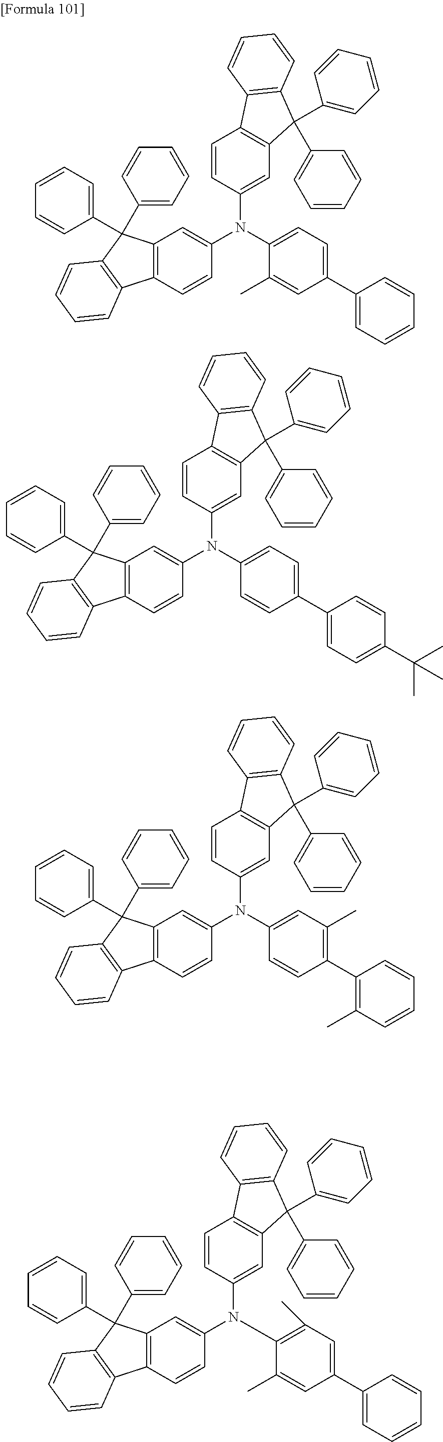 [SMILES: Cc1cc(-c2ccccc2)ccc1N(c1ccc2c(c1)C(c1ccccc1)(c1ccccc1)c1ccccc1-2)c1ccc2c(c1)C(c1ccccc1)(c1ccccc1)c1ccccc1-2 ; CC(C)(C)c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; Cc1ccccc1-c1ccc(N(c2ccc3c(c2)C(c2ccccc2)(c2ccccc2)c2ccccc2-3)c2ccc3c(c2)C(c2ccccc2)(c2ccccc2)c2ccccc2-3)cc1C ; Cc1cc(-c2ccccc2)cc(C)c1N(c1ccc2c(c1)C(c1ccccc1)(c1ccccc1)c1ccccc1-2)c1ccc2c(c1)C(c1ccccc1)(c1ccccc1)c1ccccc1-2]

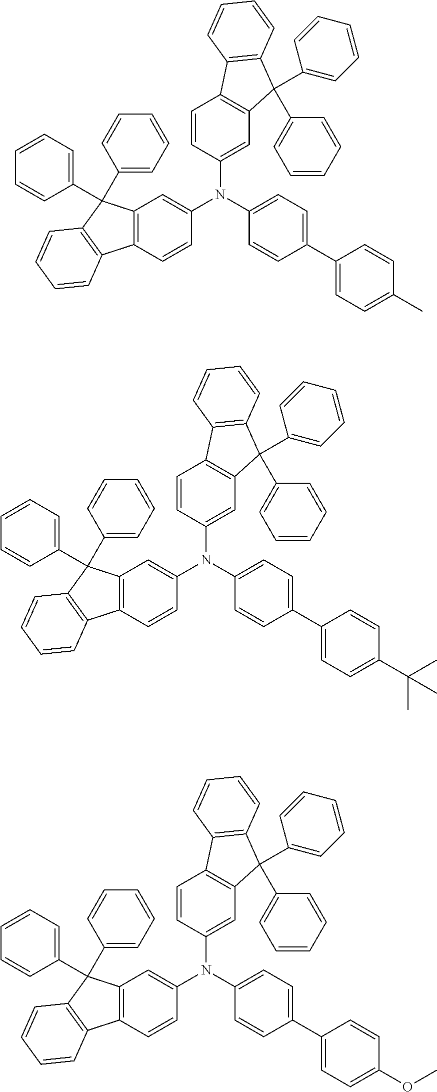 [SMILES: Cc1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; CC(C)(C)c1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1 ; COc1ccc(-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc2)cc1]

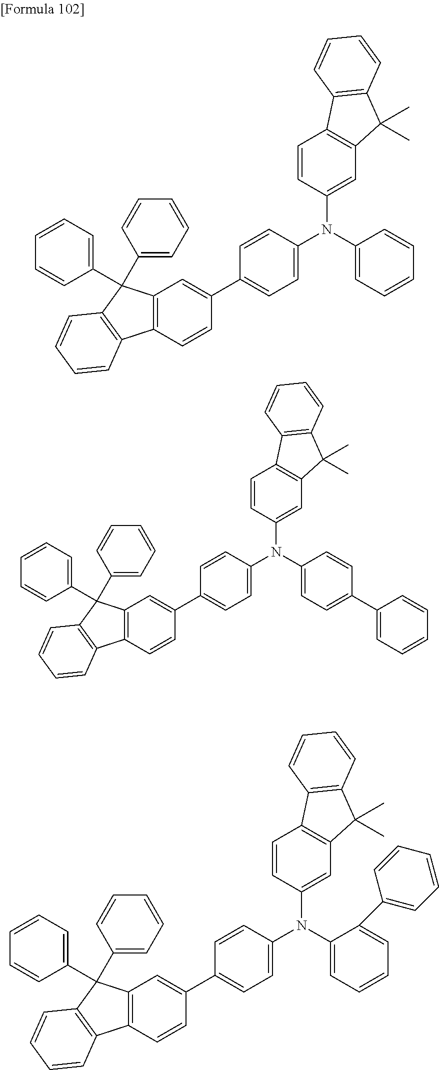 [SMILES: CC1(C)c2ccccc2-c2ccc(N(c3ccccc3)c3ccc(-c4ccc5c(c4)C(c4ccccc4)(c4ccccc4)c4ccccc4-5)cc3)cc21 ; CC1(C)c2ccccc2-c2ccc(N(c3ccc(-c4ccccc4)cc3)c3ccc(-c4ccc5c(c4)C(c4ccccc4)(c4ccccc4)c4ccccc4-5)cc3)cc21 ; CC1(C)c2ccccc2-c2ccc(N(c3ccc(-c4ccc5c(c4)C(c4ccccc4)(c4ccccc4)c4ccccc4-5)cc3)c3ccccc3-c3ccccc3)cc21]

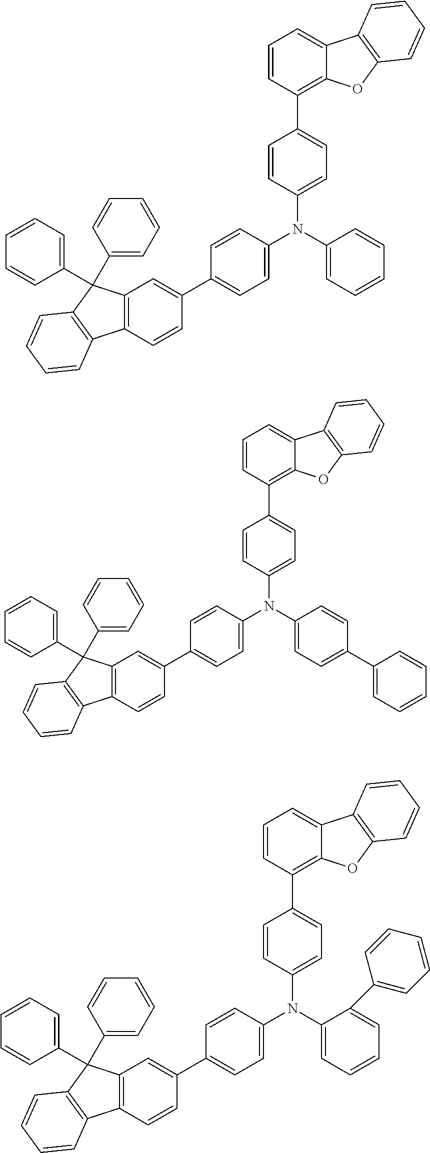 [SMILES: c1ccc(N(c2ccc(-c3ccc4c(c3)C(c3ccccc3)(c3ccccc3)c3ccccc3-4)cc2)c2ccc(-c3cccc4c3oc3ccccc34)cc2)cc1 ; c1ccc(-c2ccc(N(c3ccc(-c4ccc5c(c4)C(c4ccccc4)(c4ccccc4)c4ccccc4-5)cc3)c3ccc(-c4cccc5c4oc4ccccc45)cc3)cc2)cc1 ; c1ccc(-c2ccccc2N(c2ccc(-c3ccc4c(c3)C(c3ccccc3)(c3ccccc3)c3ccccc3-4)cc2)c2ccc(-c3cccc4c3oc3ccccc34)cc2)cc1]

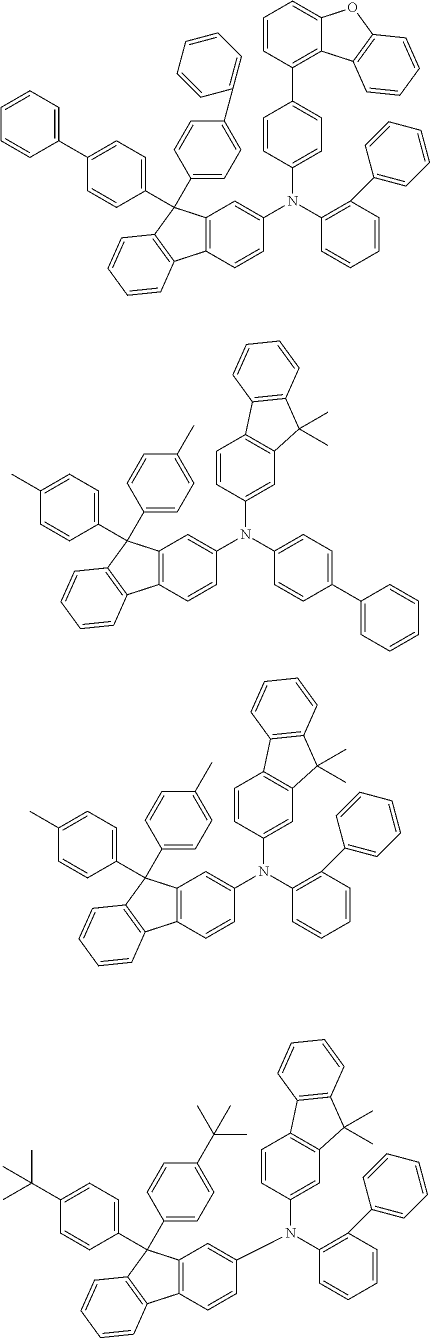 [SMILES: c1ccc(-c2ccc(C3(c4ccc(-c5ccccc5)cc4)c4ccccc4-c4ccc(N(c5ccc(-c6cccc7oc8ccccc8c67)cc5)c5ccccc5-c5ccccc5)cc43)cc2)cc1 ; Cc1ccc(C2(c3ccc(C)cc3)c3ccccc3-c3ccc(N(c4ccc(-c5ccccc5)cc4)c4ccc5c(c4)C(C)(C)c4ccccc4-5)cc32)cc1 ; Cc1ccc(C2(c3ccc(C)cc3)c3ccccc3-c3ccc(N(c4ccc5c(c4)C(C)(C)c4ccccc4-5)c4ccccc4-c4ccccc4)cc32)cc1 ; CC(C)(C)c1ccc(C2(c3ccc(C(C)(C)C)cc3)c3ccccc3-c3ccc(N(c4ccc5c(c4)C(C)(C)c4ccccc4-5)c4ccccc4-c4ccccc4)cc32)cc1]

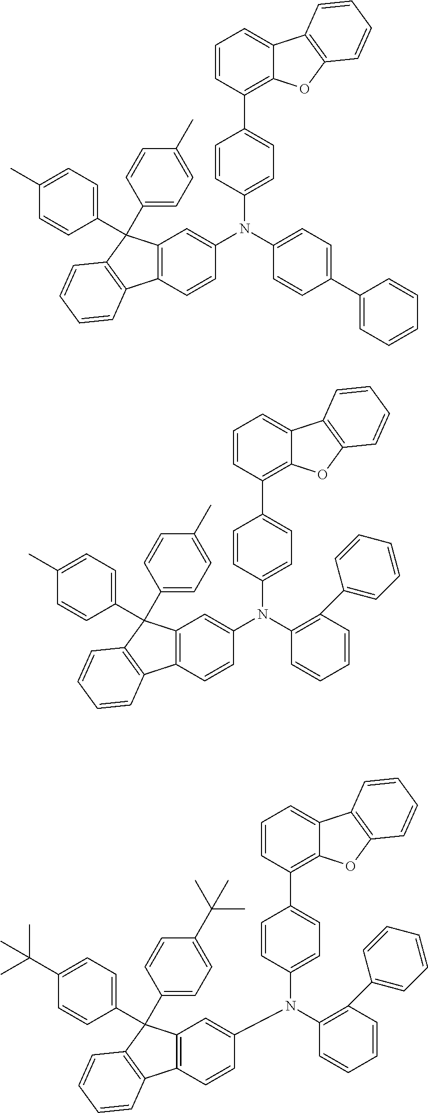

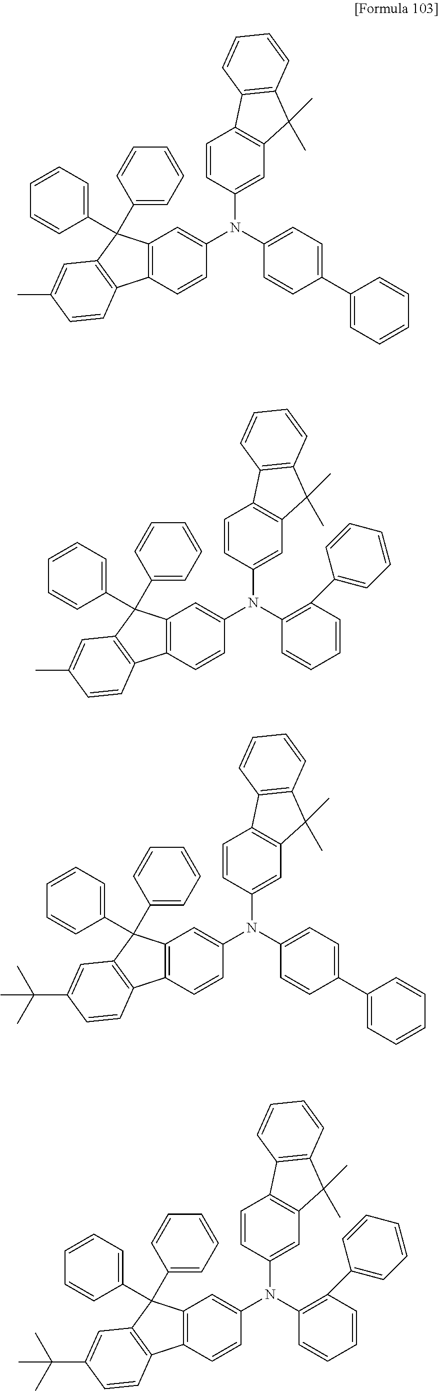 [SMILES: Cc1ccc2c(c1)C(c1ccccc1)(c1ccccc1)c1cc(N(c3ccc(-c4ccccc4)cc3)c3ccc4c(c3)C(C)(C)c3ccccc3-4)ccc1-2 ; Cc1ccc2c(c1)C(c1ccccc1)(c1ccccc1)c1cc(N(c3ccc4c(c3)C(C)(C)c3ccccc3-4)c3ccccc3-c3ccccc3)ccc1-2 ; CC(C)(C)c1ccc2c(c1)C(c1ccccc1)(c1ccccc1)c1cc(N(c3ccc(-c4ccccc4)cc3)c3ccc4c(c3)C(C)(C)c3ccccc3-4)ccc1-2 ; CC(C)(C)c1ccc2c(c1)C(c1ccccc1)(c1ccccc1)c1cc(N(c3ccc4c(c3)C(C)(C)c3ccccc3-4)c3ccccc3-c3ccccc3)ccc1-2]

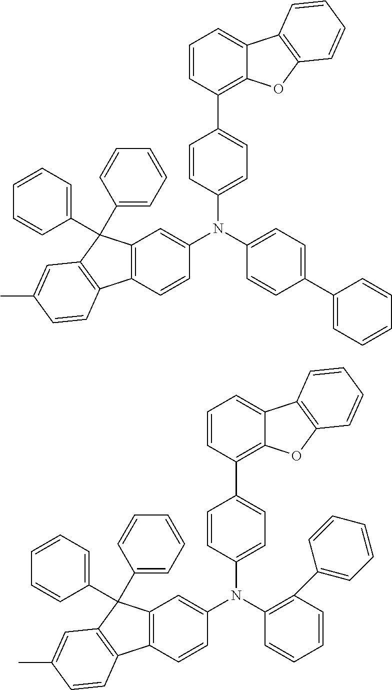

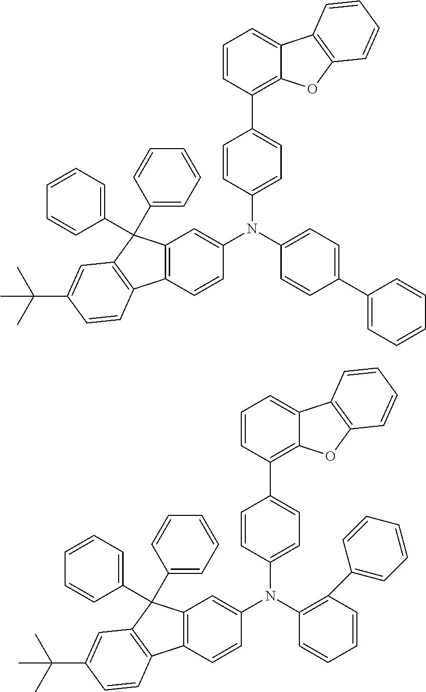

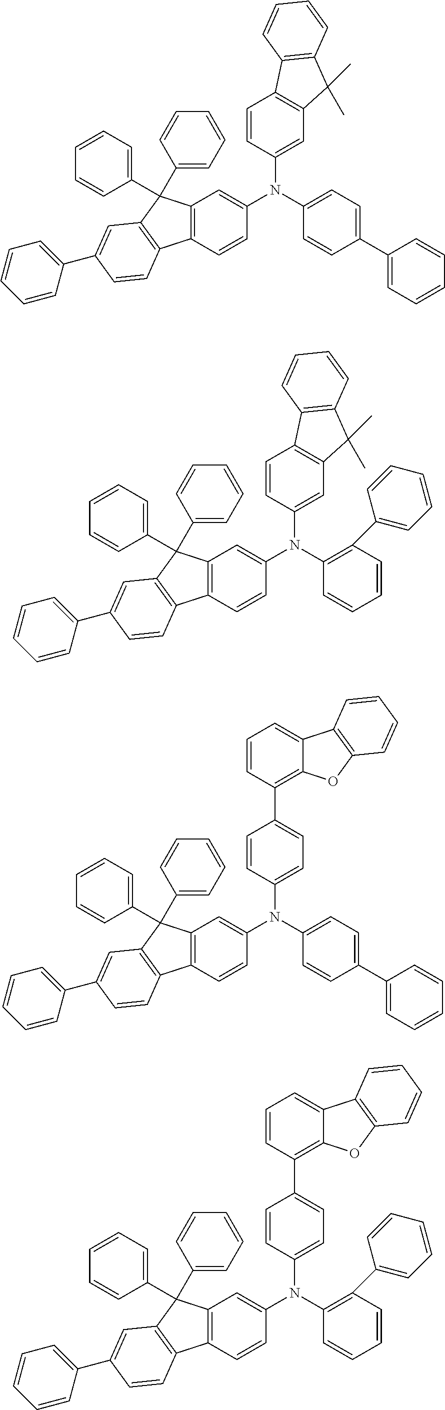

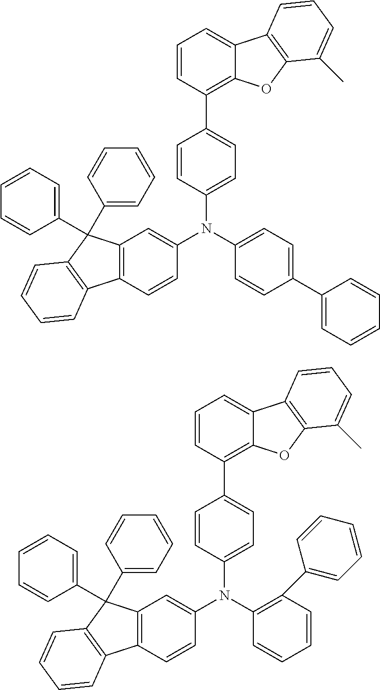

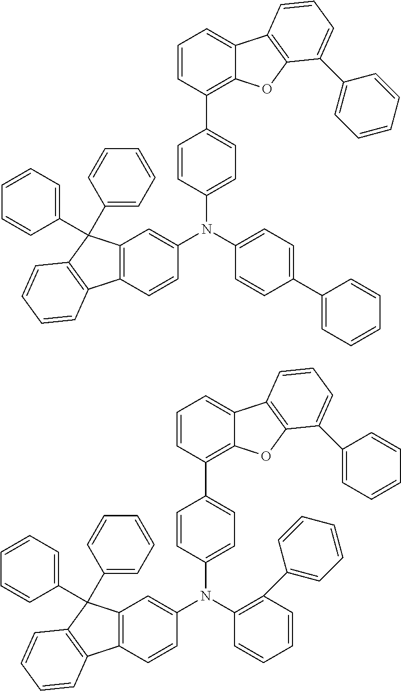

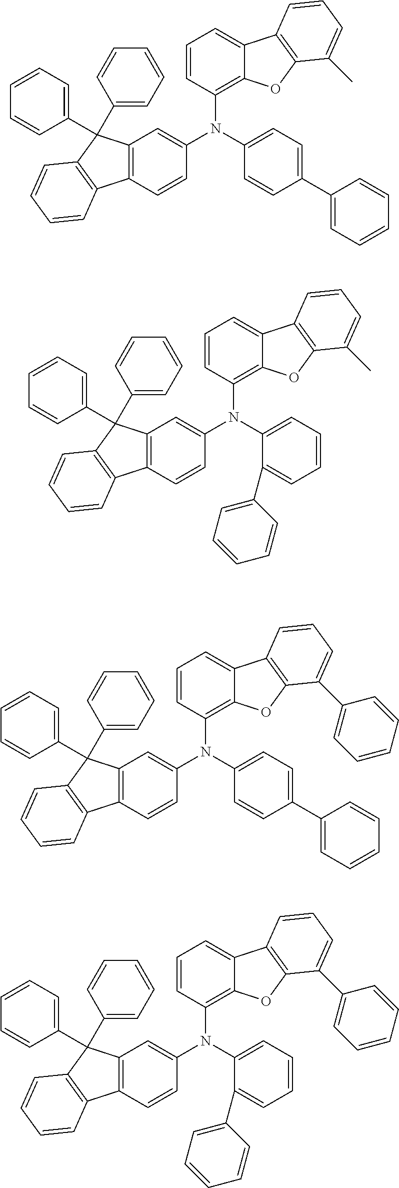 [SMILES: Cc1cccc2c1oc1c(N(c3ccc(-c4ccccc4)cc3)c3ccc4c(c3)C(c3ccccc3)(c3ccccc3)c3ccccc3-4)cccc12 ; Cc1cccc2c1oc1c(N(c3ccc4c(c3)C(c3ccccc3)(c3ccccc3)c3ccccc3-4)c3ccccc3-c3ccccc3)cccc12 ; c1ccc(-c2ccc(N(c3ccc4c(c3)C(c3ccccc3)(c3ccccc3)c3ccccc3-4)c3cccc4c3oc3c(-c5ccccc5)cccc34)cc2)cc1 ; c1ccc(-c2ccccc2N(c2ccc3c(c2)C(c2ccccc2)(c2ccccc2)c2ccccc2-3)c2cccc3c2oc2c(-c4ccccc4)cccc23)cc1]

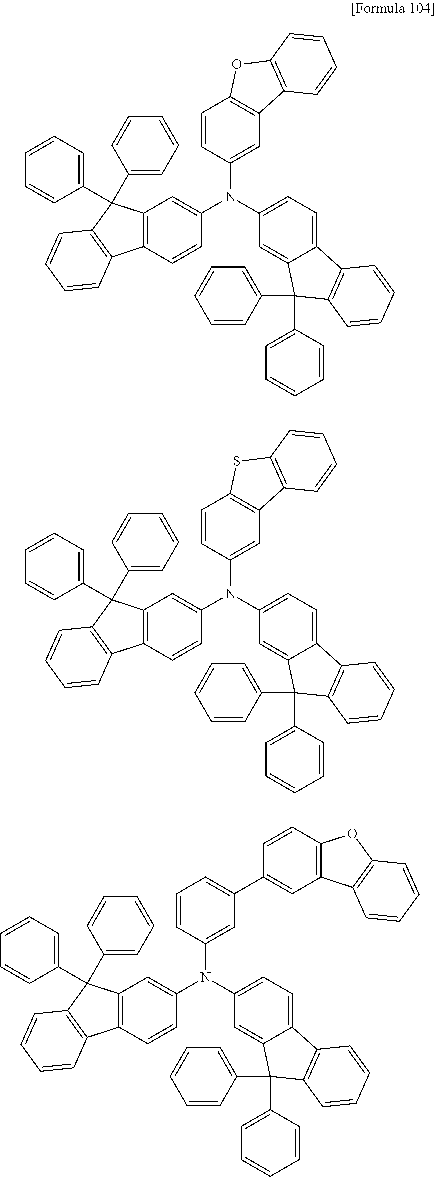

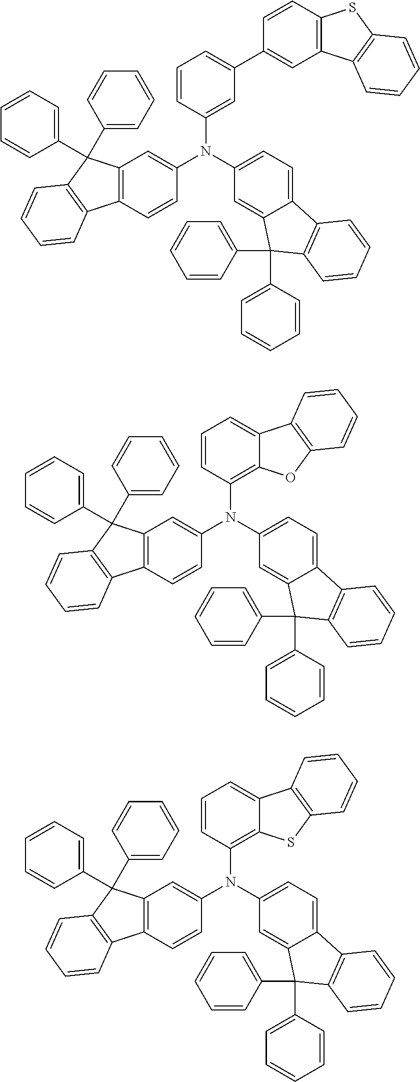

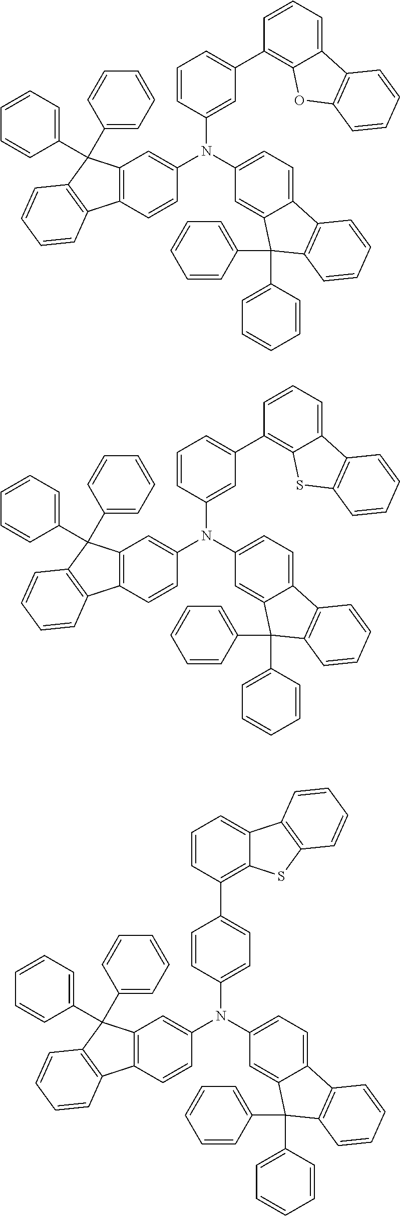

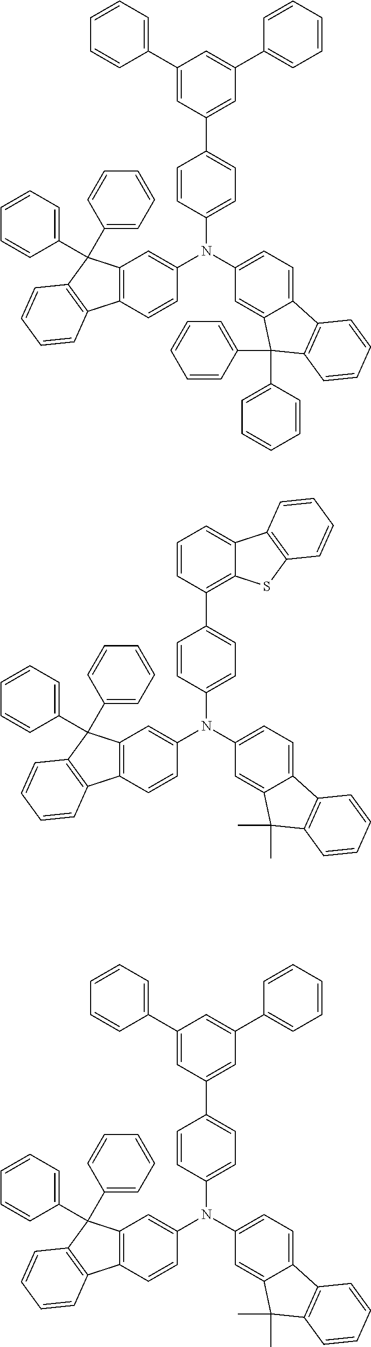 [SMILES: c1ccc(-c2cc(-c3ccccc3)cc(-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3)c2)cc1 ; CC1(C)c2ccccc2-c2ccc(N(c3ccc(-c4cccc5c4sc4ccccc45)cc3)c3ccc4c(c3)C(c3ccccc3)(c3ccccc3)c3ccccc3-4)cc21 ; CC1(C)c2ccccc2-c2ccc(N(c3ccc(-c4cc(-c5ccccc5)cc(-c5ccccc5)c4)cc3)c3ccc4c(c3)C(c3ccccc3)(c3ccccc3)c3ccccc3-4)cc21]

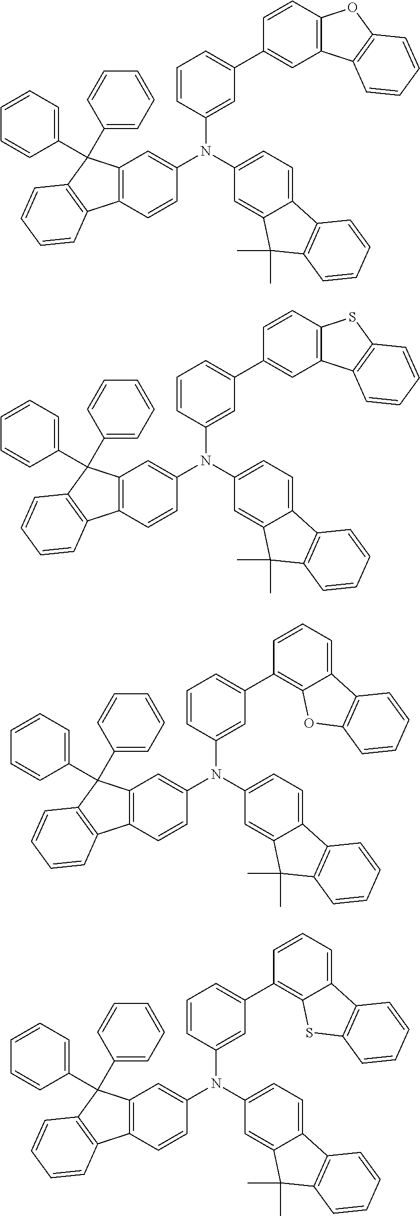

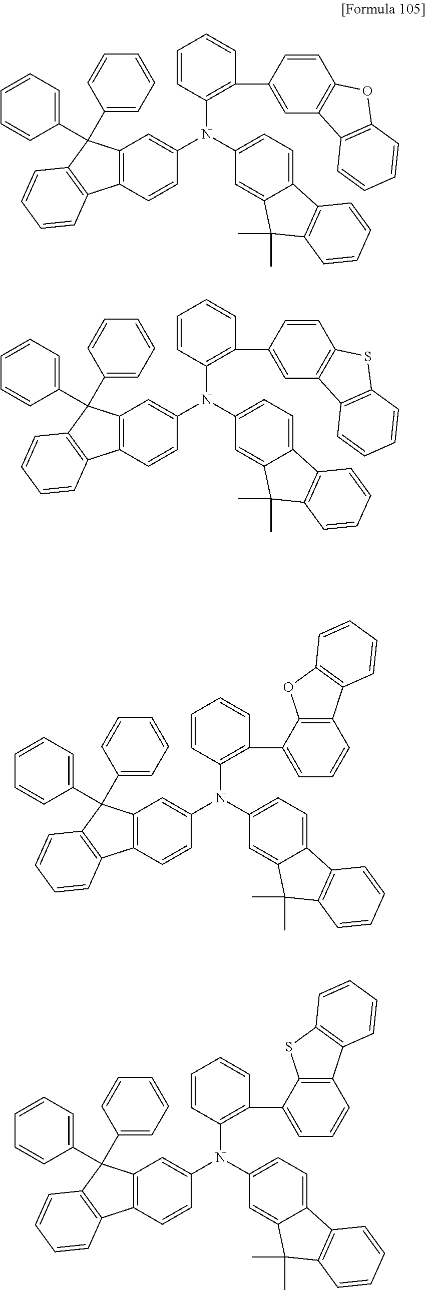 [SMILES: CC1(C)c2ccccc2-c2ccc(N(c3ccc4c(c3)C(c3ccccc3)(c3ccccc3)c3ccccc3-4)c3ccccc3-c3ccc4oc5ccccc5c4c3)cc21 ; CC1(C)c2ccccc2-c2ccc(N(c3ccc4c(c3)C(c3ccccc3)(c3ccccc3)c3ccccc3-4)c3ccccc3-c3ccc4sc5ccccc5c4c3)cc21 ; CC1(C)c2ccccc2-c2ccc(N(c3ccc4c(c3)C(c3ccccc3)(c3ccccc3)c3ccccc3-4)c3ccccc3-c3cccc4c3oc3ccccc34)cc21 ; CC1(C)c2ccccc2-c2ccc(N(c3ccc4c(c3)C(c3ccccc3)(c3ccccc3)c3ccccc3-4)c3ccccc3-c3cccc4c3sc3ccccc34)cc21]

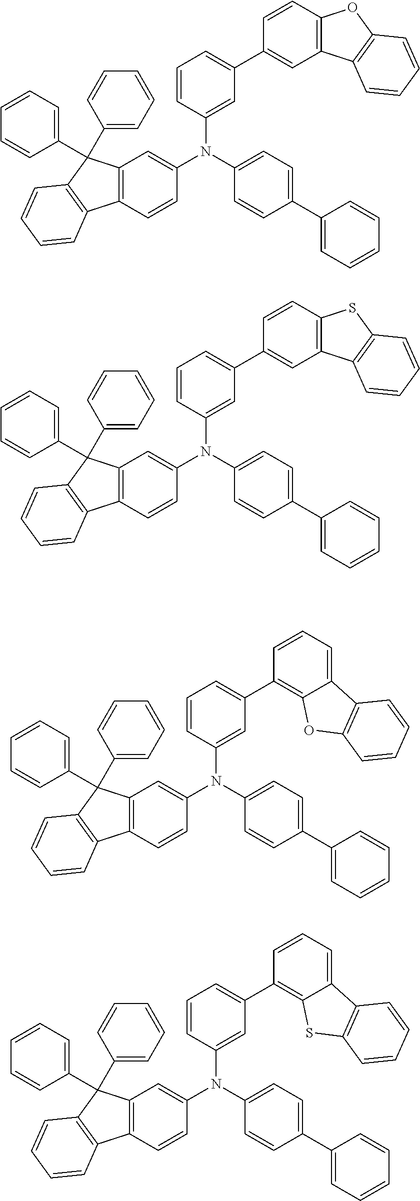

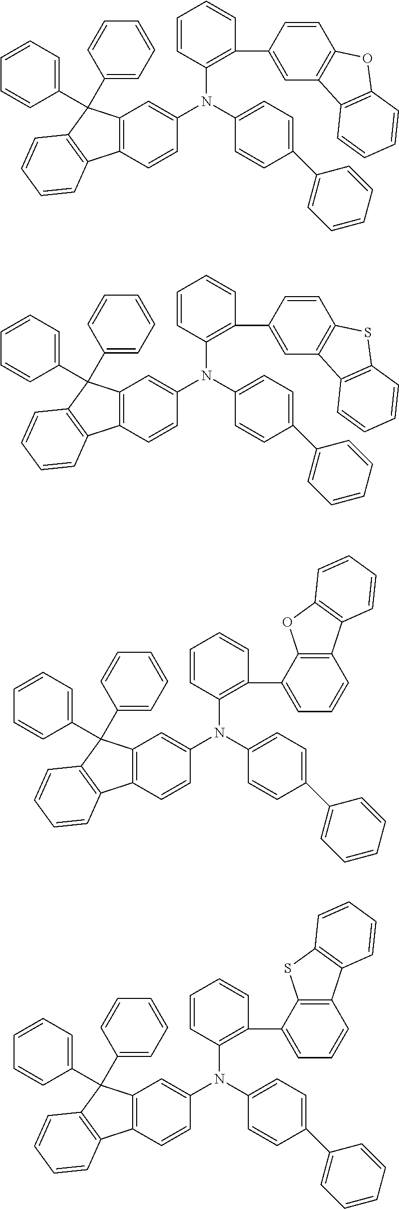

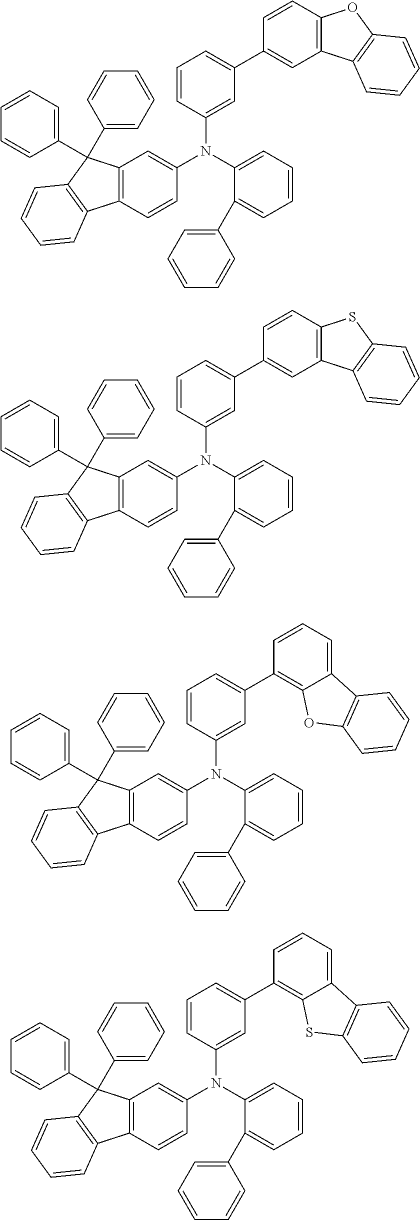 [SMILES: c1ccc(-c2ccccc2N(c2cccc(-c3ccc4oc5ccccc5c4c3)c2)c2ccc3c(c2)C(c2ccccc2)(c2ccccc2)c2ccccc2-3)cc1 ; c1ccc(-c2ccccc2N(c2cccc(-c3ccc4sc5ccccc5c4c3)c2)c2ccc3c(c2)C(c2ccccc2)(c2ccccc2)c2ccccc2-3)cc1 ; c1ccc(-c2ccccc2N(c2cccc(-c3cccc4c3oc3ccccc34)c2)c2ccc3c(c2)C(c2ccccc2)(c2ccccc2)c2ccccc2-3)cc1 ; c1ccc(-c2ccccc2N(c2cccc(-c3cccc4c3sc3ccccc34)c2)c2ccc3c(c2)C(c2ccccc2)(c2ccccc2)c2ccccc2-3)cc1]

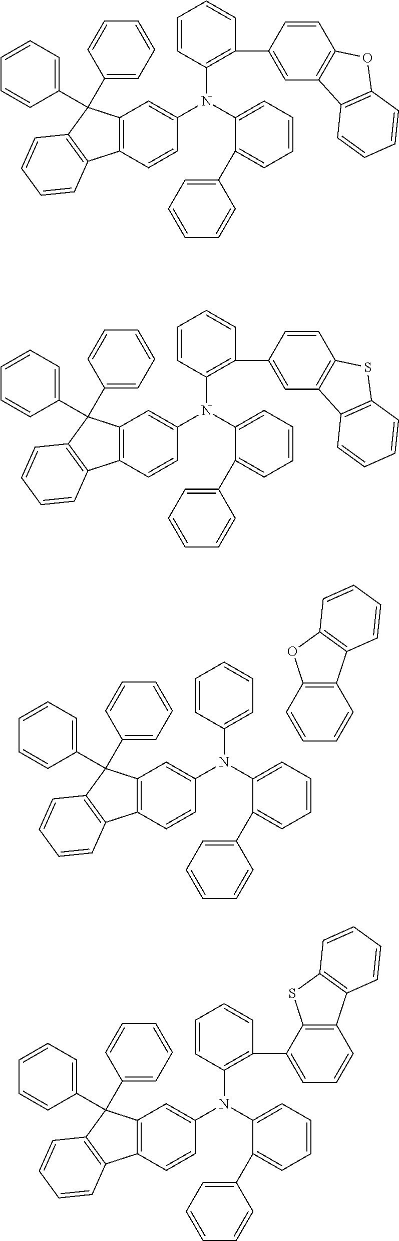

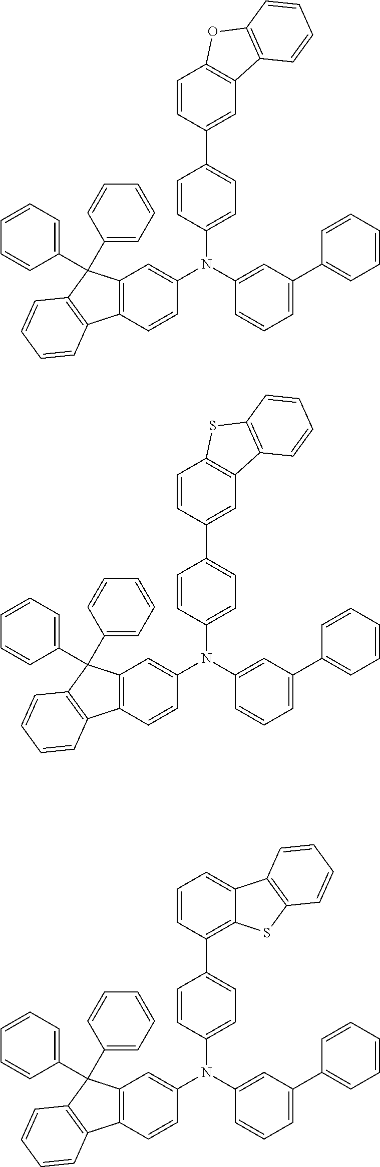 [SMILES: c1ccc(-c2cccc(N(c3ccc(-c4ccc5oc6ccccc6c5c4)cc3)c3ccc4c(c3)C(c3ccccc3)(c3ccccc3)c3ccccc3-4)c2)cc1 ; c1ccc(-c2cccc(N(c3ccc(-c4ccc5sc6ccccc6c5c4)cc3)c3ccc4c(c3)C(c3ccccc3)(c3ccccc3)c3ccccc3-4)c2)cc1 ; c1ccc(-c2cccc(N(c3ccc(-c4cccc5c4sc4ccccc45)cc3)c3ccc4c(c3)C(c3ccccc3)(c3ccccc3)c3ccccc3-4)c2)cc1]

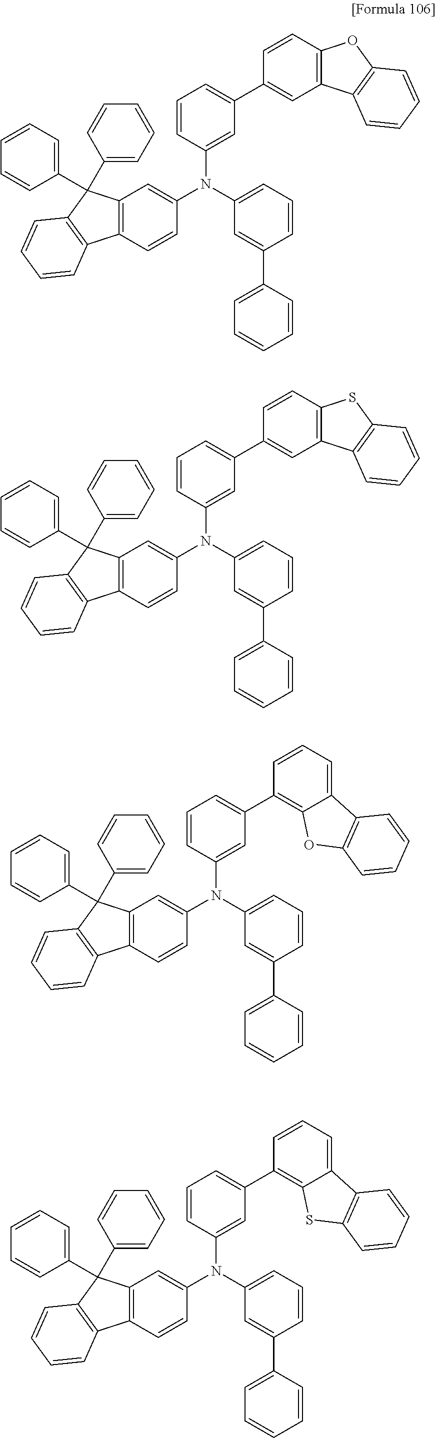 [SMILES: c1ccc(-c2cccc(N(c3cccc(-c4ccc5oc6ccccc6c5c4)c3)c3ccc4c(c3)C(c3ccccc3)(c3ccccc3)c3ccccc3-4)c2)cc1 ; c1ccc(-c2cccc(N(c3cccc(-c4ccc5sc6ccccc6c5c4)c3)c3ccc4c(c3)C(c3ccccc3)(c3ccccc3)c3ccccc3-4)c2)cc1 ; c1ccc(-c2cccc(N(c3cccc(-c4cccc5c4oc4ccccc45)c3)c3ccc4c(c3)C(c3ccccc3)(c3ccccc3)c3ccccc3-4)c2)cc1 ; c1ccc(-c2cccc(N(c3cccc(-c4cccc5c4sc4ccccc45)c3)c3ccc4c(c3)C(c3ccccc3)(c3ccccc3)c3ccccc3-4)c2)cc1]

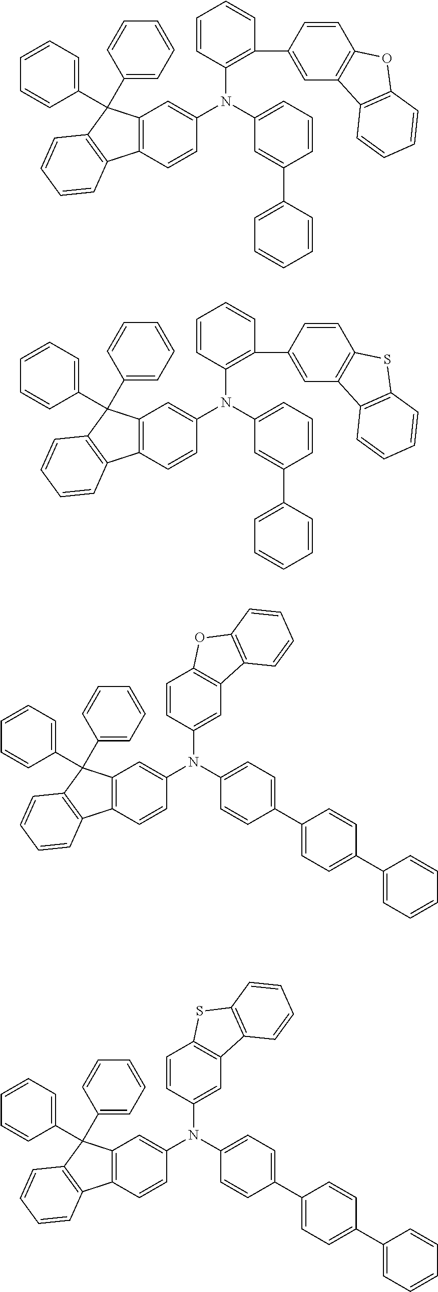

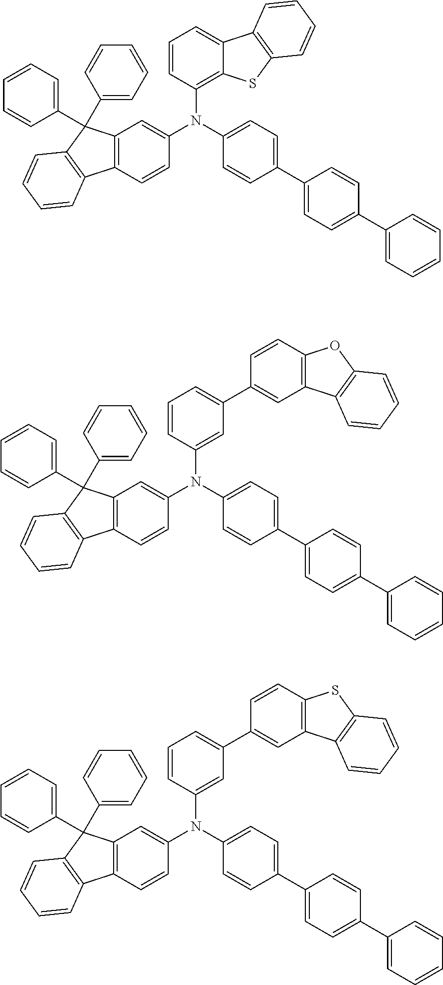 [SMILES: c1ccc(-c2ccc(-c3ccc(N(c4ccc5c(c4)C(c4ccccc4)(c4ccccc4)c4ccccc4-5)c4cccc5c4sc4ccccc45)cc3)cc2)cc1 ; c1ccc(-c2ccc(-c3ccc(N(c4cccc(-c5ccc6oc7ccccc7c6c5)c4)c4ccc5c(c4)C(c4ccccc4)(c4ccccc4)c4ccccc4-5)cc3)cc2)cc1 ; c1ccc(-c2ccc(-c3ccc(N(c4cccc(-c5ccc6sc7ccccc7c6c5)c4)c4ccc5c(c4)C(c4ccccc4)(c4ccccc4)c4ccccc4-5)cc3)cc2)cc1]

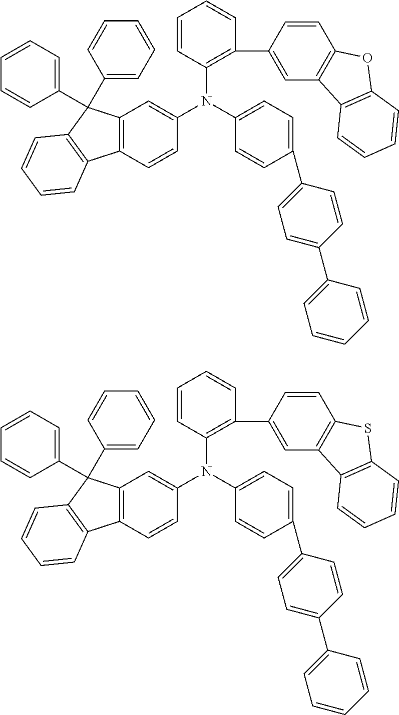 [SMILES: c1ccc(-c2ccc(-c3ccc(N(c4ccc5c(c4)C(c4ccccc4)(c4ccccc4)c4ccccc4-5)c4ccccc4-c4ccc5oc6ccccc6c5c4)cc3)cc2)cc1 ; c1ccc(-c2ccc(-c3ccc(N(c4ccc5c(c4)C(c4ccccc4)(c4ccccc4)c4ccccc4-5)c4ccccc4-c4ccc5sc6ccccc6c5c4)cc3)cc2)cc1]

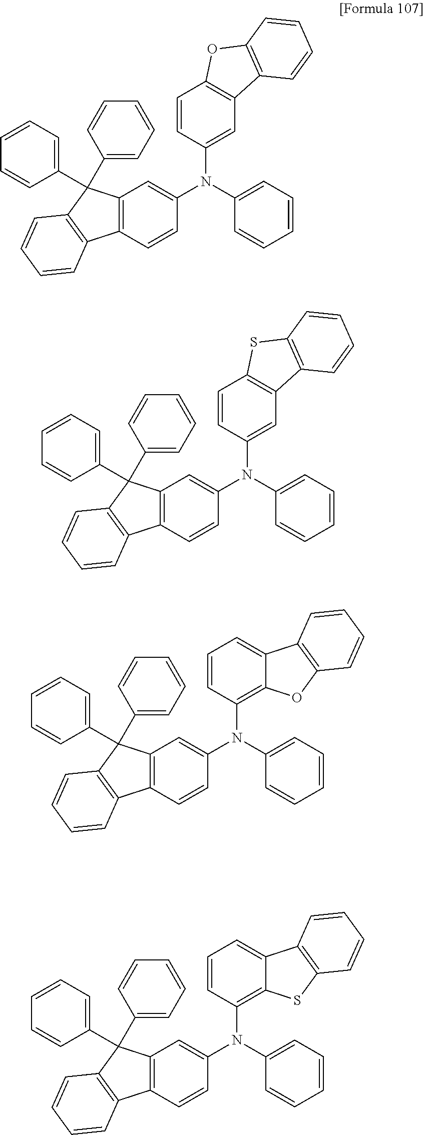 [SMILES: c1ccc(N(c2ccc3c(c2)C(c2ccccc2)(c2ccccc2)c2ccccc2-3)c2ccc3oc4ccccc4c3c2)cc1 ; c1ccc(N(c2ccc3c(c2)C(c2ccccc2)(c2ccccc2)c2ccccc2-3)c2ccc3sc4ccccc4c3c2)cc1 ; c1ccc(N(c2ccc3c(c2)C(c2ccccc2)(c2ccccc2)c2ccccc2-3)c2cccc3c2oc2ccccc23)cc1 ; c1ccc(N(c2ccc3c(c2)C(c2ccccc2)(c2ccccc2)c2ccccc2-3)c2cccc3c2sc2ccccc23)cc1]

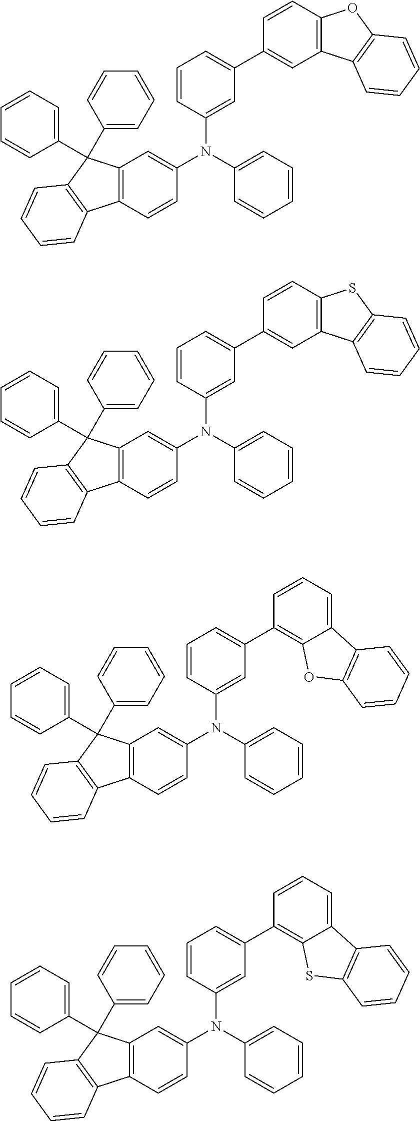 [SMILES: c1ccc(N(c2cccc(-c3ccc4oc5ccccc5c4c3)c2)c2ccc3c(c2)C(c2ccccc2)(c2ccccc2)c2ccccc2-3)cc1 ; c1ccc(N(c2cccc(-c3ccc4sc5ccccc5c4c3)c2)c2ccc3c(c2)C(c2ccccc2)(c2ccccc2)c2ccccc2-3)cc1 ; c1ccc(N(c2cccc(-c3cccc4c3oc3ccccc34)c2)c2ccc3c(c2)C(c2ccccc2)(c2ccccc2)c2ccccc2-3)cc1 ; c1ccc(N(c2cccc(-c3cccc4c3sc3ccccc34)c2)c2ccc3c(c2)C(c2ccccc2)(c2ccccc2)c2ccccc2-3)cc1]

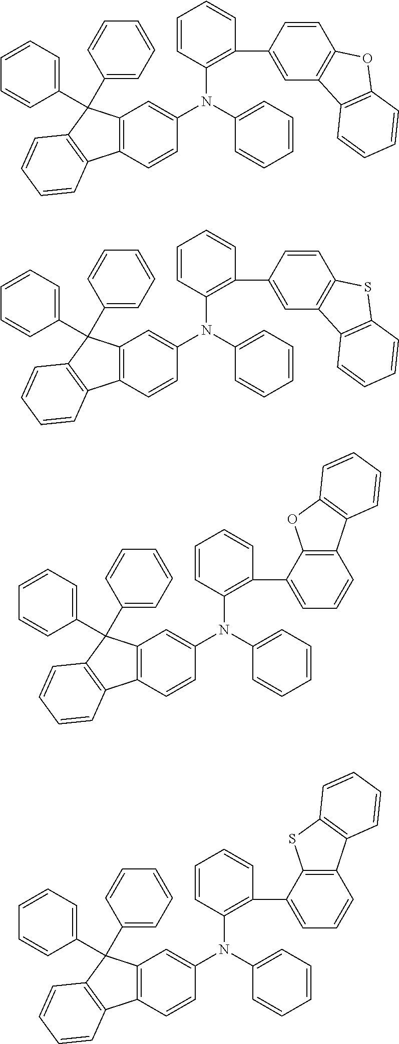 [SMILES: c1ccc(N(c2ccc3c(c2)C(c2ccccc2)(c2ccccc2)c2ccccc2-3)c2ccccc2-c2ccc3oc4ccccc4c3c2)cc1 ; c1ccc(N(c2ccc3c(c2)C(c2ccccc2)(c2ccccc2)c2ccccc2-3)c2ccccc2-c2ccc3sc4ccccc4c3c2)cc1 ; c1ccc(N(c2ccc3c(c2)C(c2ccccc2)(c2ccccc2)c2ccccc2-3)c2ccccc2-c2cccc3c2oc2ccccc23)cc1 ; c1ccc(N(c2ccc3c(c2)C(c2ccccc2)(c2ccccc2)c2ccccc2-3)c2ccccc2-c2cccc3c2sc2ccccc23)cc1]

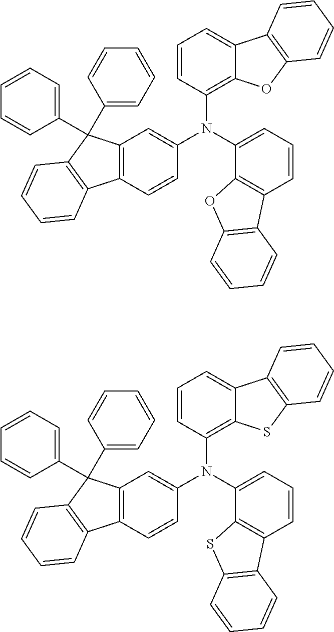 [SMILES: c1ccc(C2(c3ccccc3)c3ccccc3-c3ccc(N(c4cccc5c4oc4ccccc45)c4cccc5c4oc4ccccc45)cc32)cc1 ; c1ccc(C2(c3ccccc3)c3ccccc3-c3ccc(N(c4cccc5c4sc4ccccc45)c4cccc5c4sc4ccccc45)cc32)cc1]

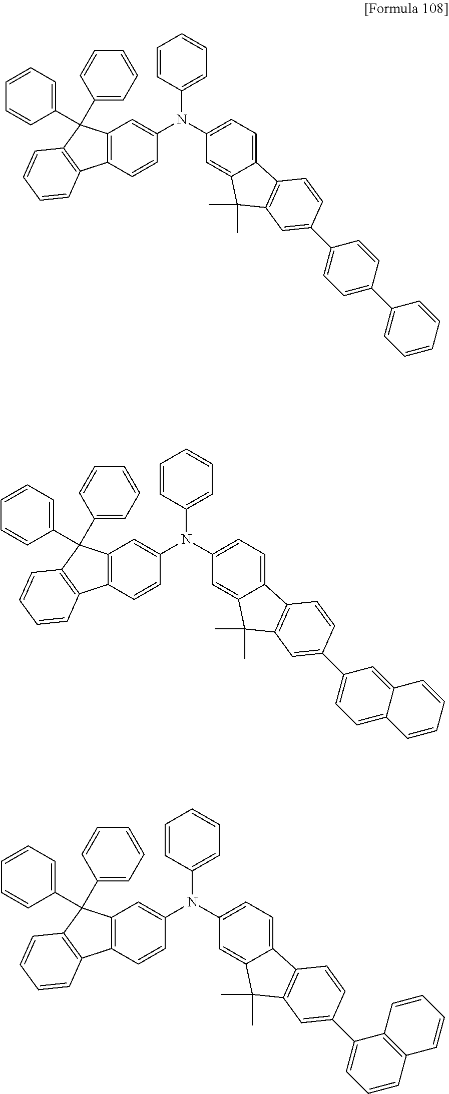 [SMILES: CC1(C)c2cc(-c3ccc(-c4ccccc4)cc3)ccc2-c2ccc(N(c3ccccc3)c3ccc4c(c3)C(c3ccccc3)(c3ccccc3)c3ccccc3-4)cc21 ; CC1(C)c2cc(-c3ccc4ccccc4c3)ccc2-c2ccc(N(c3ccccc3)c3ccc4c(c3)C(c3ccccc3)(c3ccccc3)c3ccccc3-4)cc21 ; CC1(C)c2cc(-c3cccc4ccccc34)ccc2-c2ccc(N(c3ccccc3)c3ccc4c(c3)C(c3ccccc3)(c3ccccc3)c3ccccc3-4)cc21]

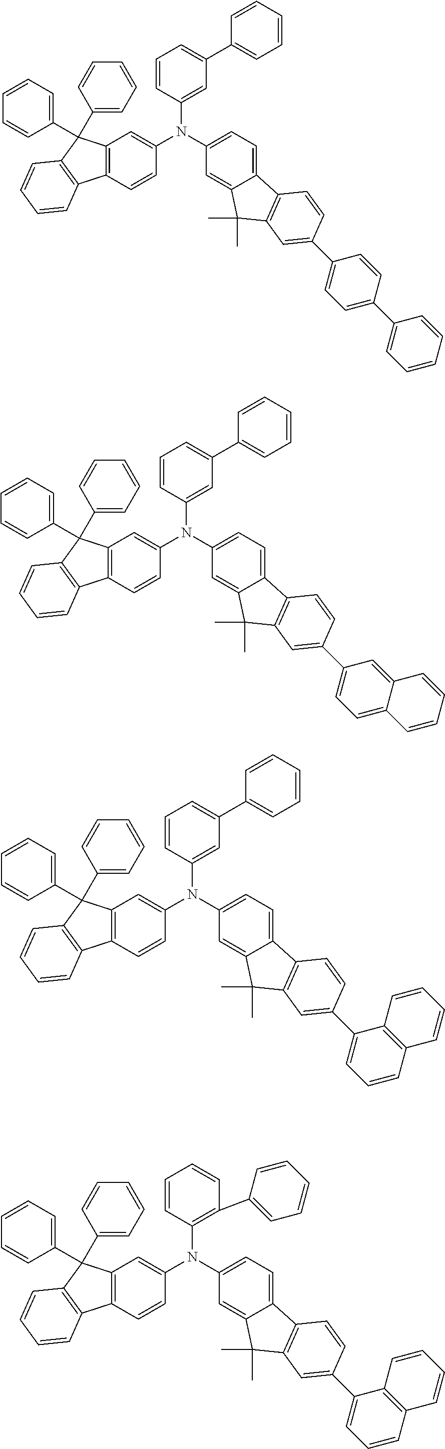 [SMILES: CC1(C)c2cc(-c3ccc(-c4ccccc4)cc3)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3ccc4ccccc4c3)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3cccc4ccccc34)ccc2-c2ccc(N(c3cccc(-c4ccccc4)c3)c3ccc4c(c3)C(c3ccccc3)(c3ccccc3)c3ccccc3-4)cc21 ; CC1(C)c2cc(-c3cccc4ccccc34)ccc2-c2ccc(N(c3ccc4c(c3)C(c3ccccc3)(c3ccccc3)c3ccccc3-4)c3ccccc3-c3ccccc3)cc21]

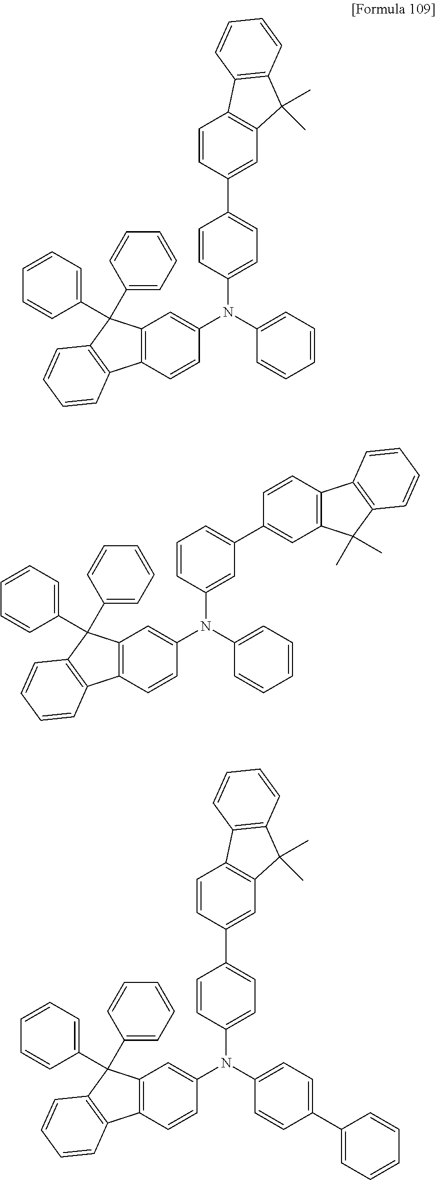 [SMILES: CC1(C)c2ccccc2-c2ccc(-c3ccc(N(c4ccccc4)c4ccc5c(c4)C(c4ccccc4)(c4ccccc4)c4ccccc4-5)cc3)cc21 ; CC1(C)c2ccccc2-c2ccc(-c3cccc(N(c4ccccc4)c4ccc5c(c4)C(c4ccccc4)(c4ccccc4)c4ccccc4-5)c3)cc21 ; CC1(C)c2ccccc2-c2ccc(-c3ccc(N(c4ccc(-c5ccccc5)cc4)c4ccc5c(c4)C(c4ccccc4)(c4ccccc4)c4ccccc4-5)cc3)cc21]

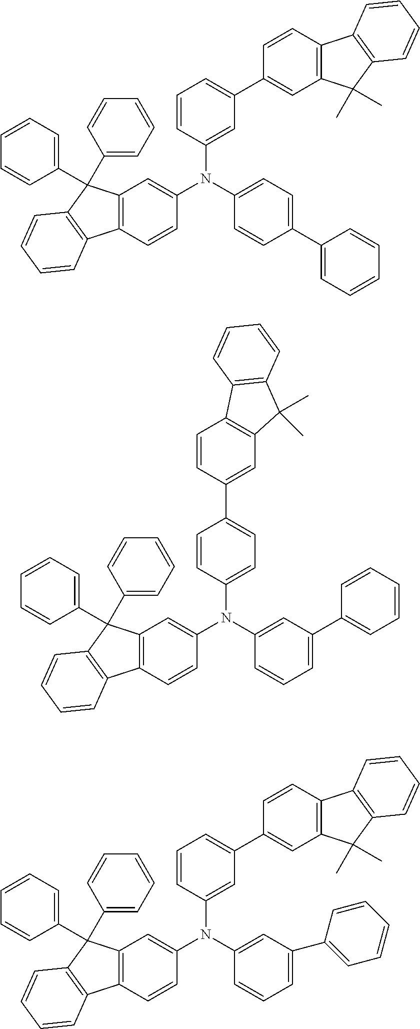 [SMILES: CC1(C)c2ccccc2-c2ccc(-c3cccc(N(c4ccc(-c5ccccc5)cc4)c4ccc5c(c4)C(c4ccccc4)(c4ccccc4)c4ccccc4-5)c3)cc21 ; CC1(C)c2ccccc2-c2ccc(-c3ccc(N(c4cccc(-c5ccccc5)c4)c4ccc5c(c4)C(c4ccccc4)(c4ccccc4)c4ccccc4-5)cc3)cc21 ; CC1(C)c2ccccc2-c2ccc(-c3cccc(N(c4cccc(-c5ccccc5)c4)c4ccc5c(c4)C(c4ccccc4)(c4ccccc4)c4ccccc4-5)c3)cc21]

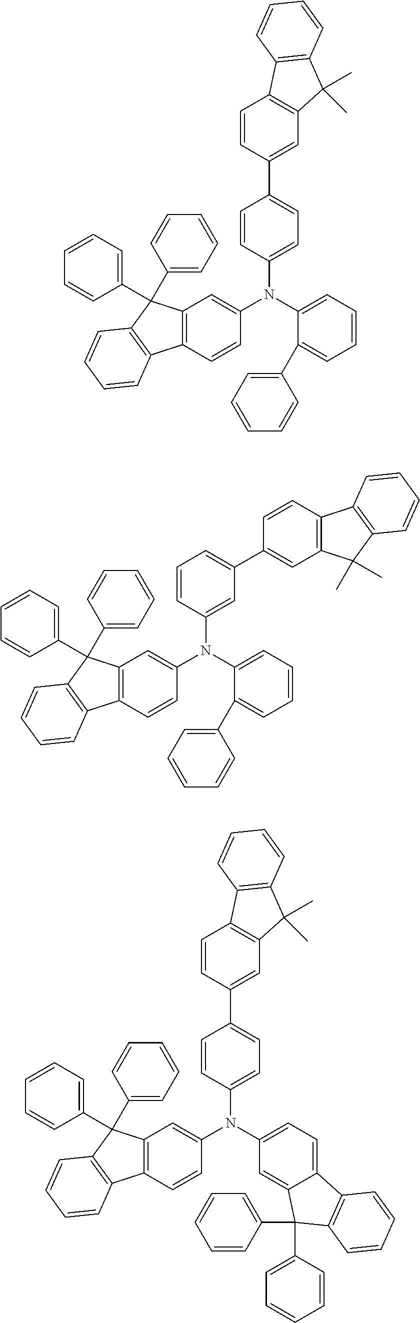 [SMILES: CC1(C)c2ccccc2-c2ccc(-c3ccc(N(c4ccc5c(c4)C(c4ccccc4)(c4ccccc4)c4ccccc4-5)c4ccccc4-c4ccccc4)cc3)cc21 ; CC1(C)c2ccccc2-c2ccc(-c3cccc(N(c4ccc5c(c4)C(c4ccccc4)(c4ccccc4)c4ccccc4-5)c4ccccc4-c4ccccc4)c3)cc21 ; CC1(C)c2ccccc2-c2ccc(-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3)cc21]

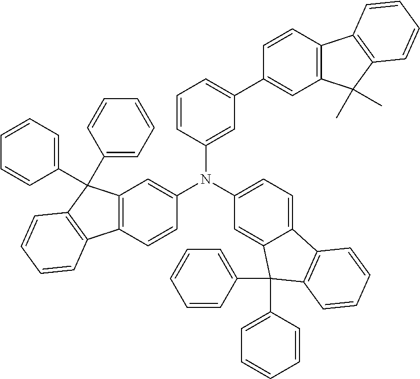 [SMILES: CC1(C)c2ccccc2-c2ccc(-c3cccc(N(c4ccc5c(c4)C(c4ccccc4)(c4ccccc4)c4ccccc4-5)c4ccc5c(c4)C(c4ccccc4)(c4ccccc4)c4ccccc4-5)c3)cc21]

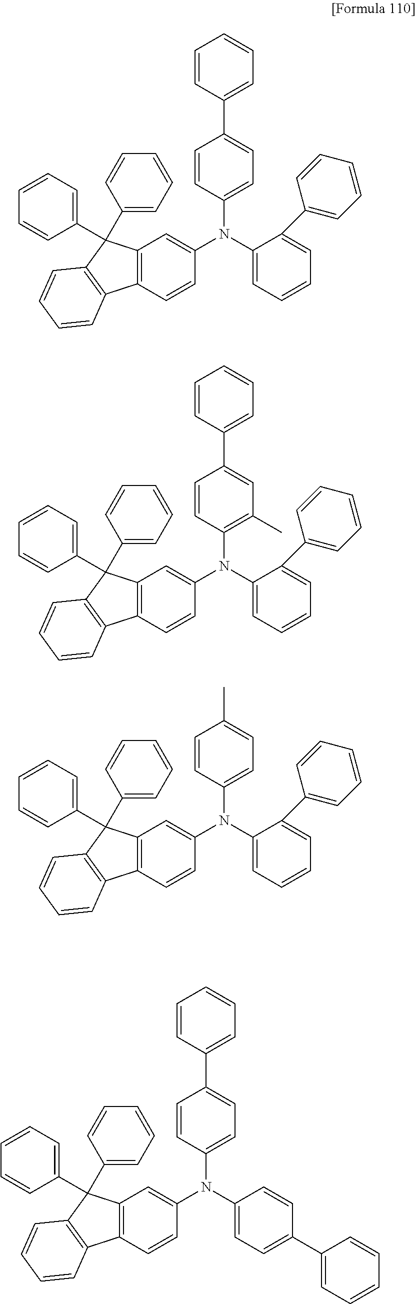 [SMILES: c1ccc(-c2ccc(N(c3ccc4c(c3)C(c3ccccc3)(c3ccccc3)c3ccccc3-4)c3ccccc3-c3ccccc3)cc2)cc1 ; Cc1cc(-c2ccccc2)ccc1N(c1ccc2c(c1)C(c1ccccc1)(c1ccccc1)c1ccccc1-2)c1ccccc1-c1ccccc1 ; Cc1ccc(N(c2ccc3c(c2)C(c2ccccc2)(c2ccccc2)c2ccccc2-3)c2ccccc2-c2ccccc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1]

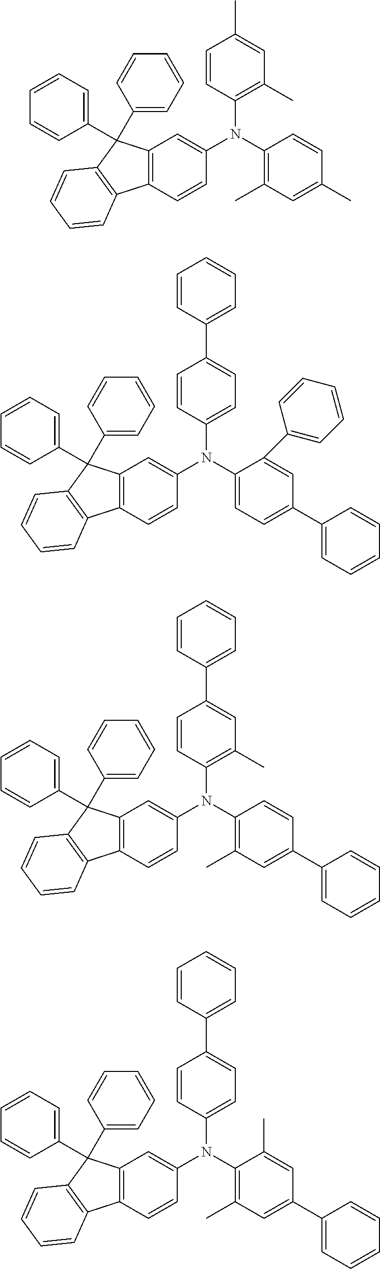 [SMILES: Cc1ccc(N(c2ccc3c(c2)C(c2ccccc2)(c2ccccc2)c2ccccc2-3)c2ccc(C)cc2C)c(C)c1 ; c1ccc(-c2ccc(N(c3ccc4c(c3)C(c3ccccc3)(c3ccccc3)c3ccccc3-4)c3ccc(-c4ccccc4)cc3-c3ccccc3)cc2)cc1 ; Cc1cc(-c2ccccc2)ccc1N(c1ccc2c(c1)C(c1ccccc1)(c1ccccc1)c1ccccc1-2)c1ccc(-c2ccccc2)cc1C ; Cc1cc(-c2ccccc2)cc(C)c1N(c1ccc(-c2ccccc2)cc1)c1ccc2c(c1)C(c1ccccc1)(c1ccccc1)c1ccccc1-2]

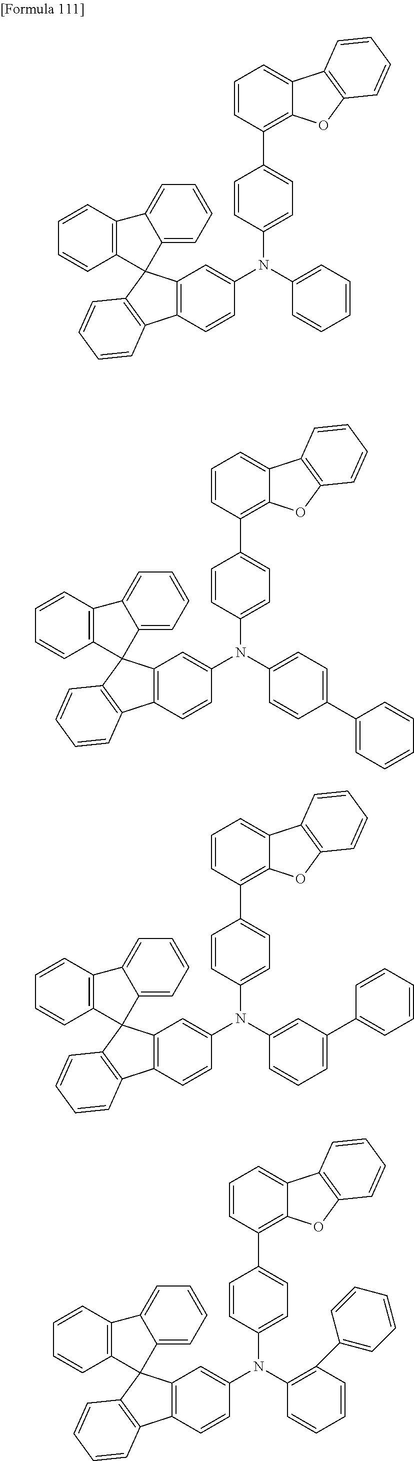

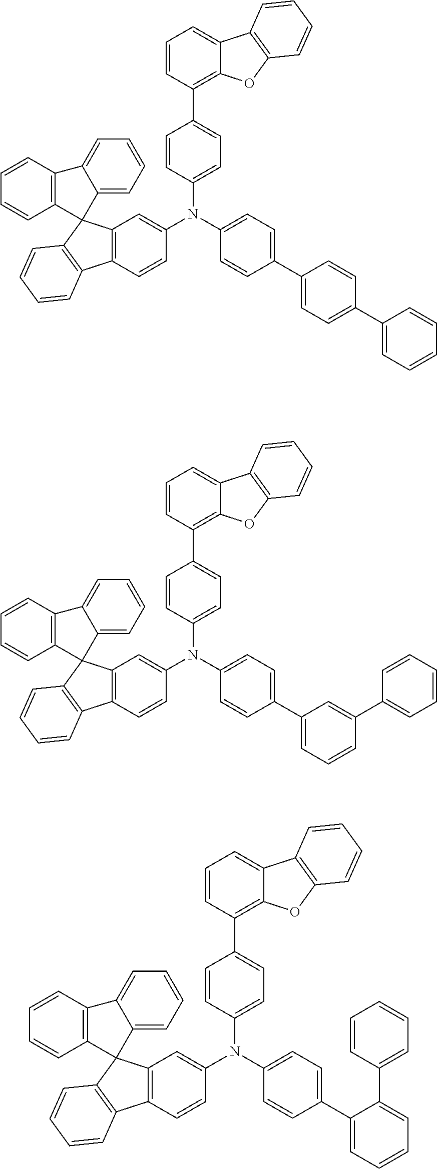 [SMILES: c1ccc(-c2ccc(-c3ccc(N(c4ccc(-c5cccc6c5oc5ccccc56)cc4)c4ccc5c(c4)C4(c6ccccc6-c6ccccc64)c4ccccc4-5)cc3)cc2)cc1 ; c1ccc(-c2cccc(-c3ccc(N(c4ccc(-c5cccc6c5oc5ccccc56)cc4)c4ccc5c(c4)C4(c6ccccc6-c6ccccc64)c4ccccc4-5)cc3)c2)cc1 ; c1ccc(-c2ccccc2-c2ccc(N(c3ccc(-c4cccc5c4oc4ccccc45)cc3)c3ccc4c(c3)C3(c5ccccc5-c5ccccc53)c3ccccc3-4)cc2)cc1]

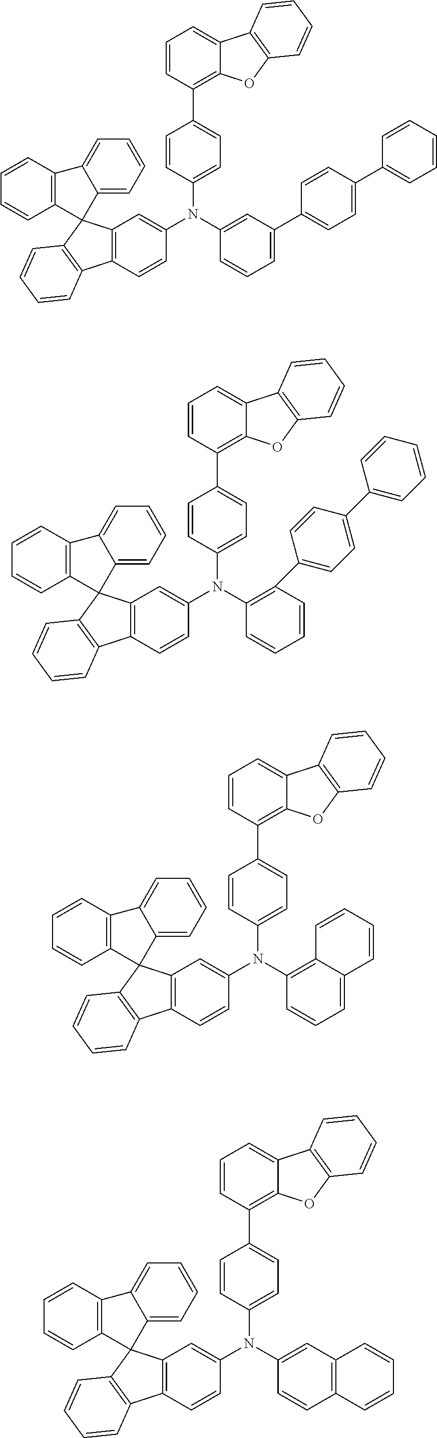 [SMILES: c1ccc(-c2ccc(-c3cccc(N(c4ccc(-c5cccc6c5oc5ccccc56)cc4)c4ccc5c(c4)C4(c6ccccc6-c6ccccc64)c4ccccc4-5)c3)cc2)cc1 ; c1ccc(-c2ccc(-c3ccccc3N(c3ccc(-c4cccc5c4oc4ccccc45)cc3)c3ccc4c(c3)C3(c5ccccc5-c5ccccc53)c3ccccc3-4)cc2)cc1 ; c1ccc2c(c1)-c1ccccc1C21c2ccccc2-c2ccc(N(c3ccc(-c4cccc5c4oc4ccccc45)cc3)c3cccc4ccccc34)cc21 ; c1ccc2c(c1)-c1ccccc1C21c2ccccc2-c2ccc(N(c3ccc(-c4cccc5c4oc4ccccc45)cc3)c3ccc4ccccc4c3)cc21]

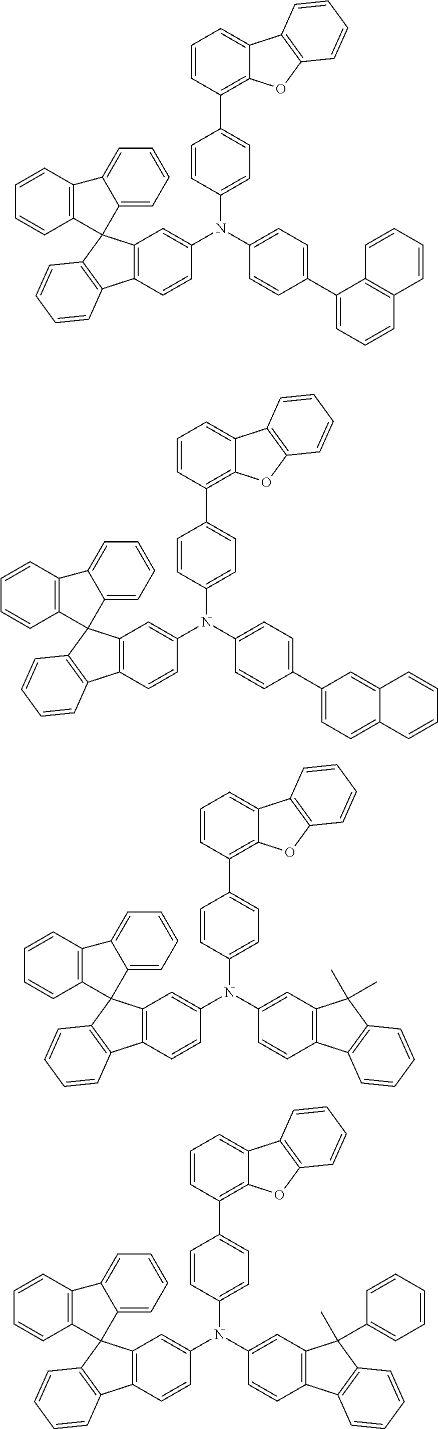

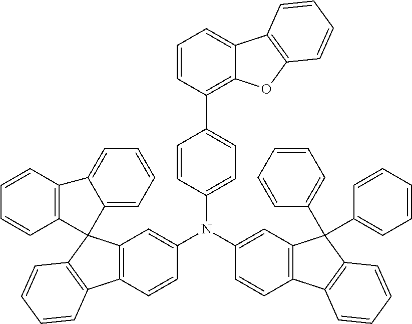 [SMILES: c1ccc(C2(c3ccccc3)c3ccccc3-c3ccc(N(c4ccc(-c5cccc6c5oc5ccccc56)cc4)c4ccc5c(c4)C4(c6ccccc6-c6ccccc64)c4ccccc4-5)cc32)cc1]

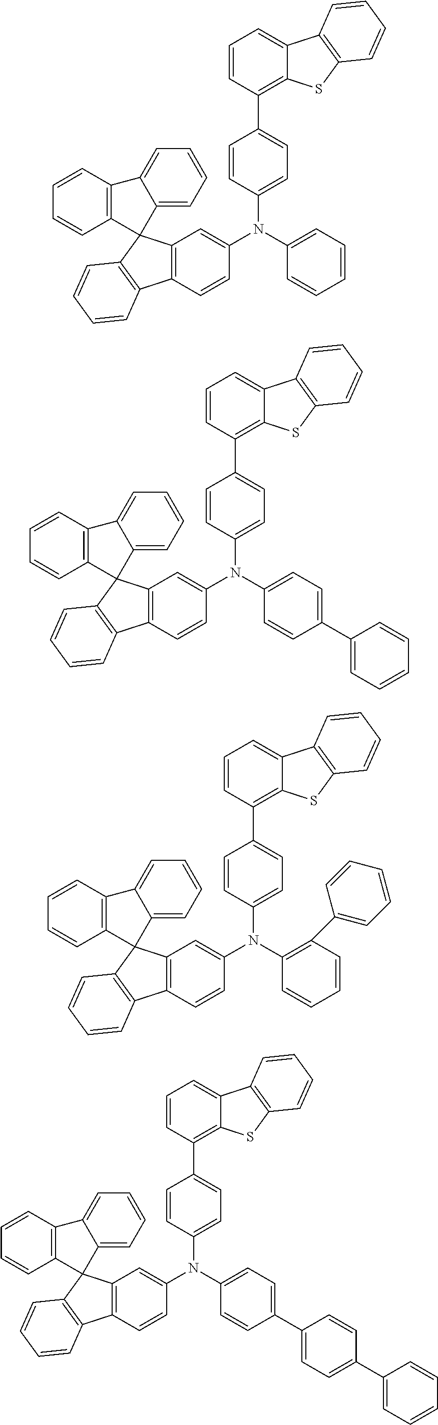

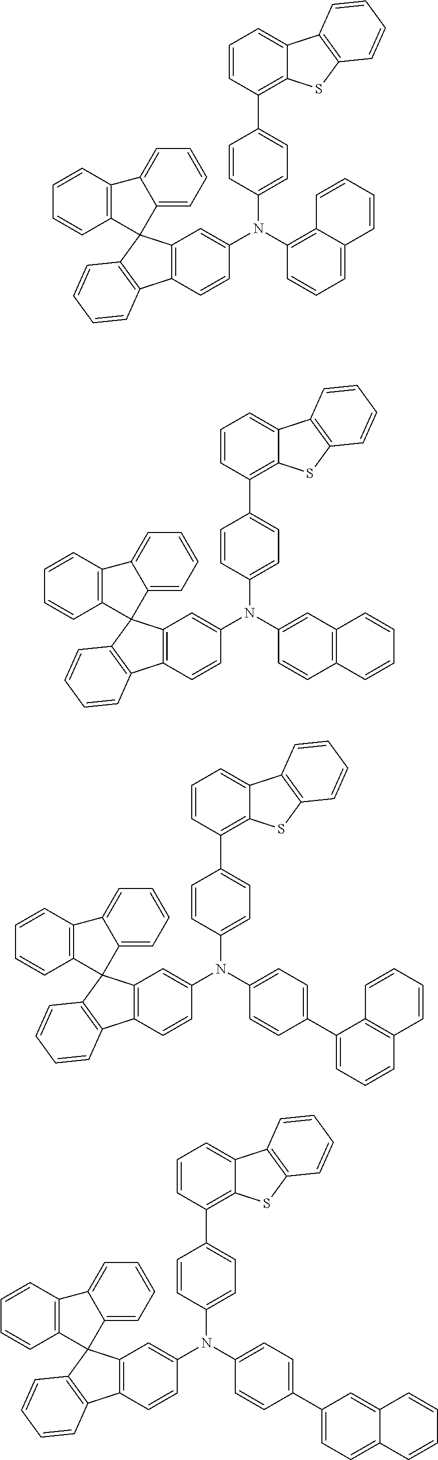 [SMILES: c1ccc2c(c1)-c1ccccc1C21c2ccccc2-c2ccc(N(c3ccc(-c4cccc5c4sc4ccccc45)cc3)c3cccc4ccccc34)cc21 ; c1ccc2c(c1)-c1ccccc1C21c2ccccc2-c2ccc(N(c3ccc(-c4cccc5c4sc4ccccc45)cc3)c3ccc4ccccc4c3)cc21 ; c1ccc2c(c1)-c1ccccc1C21c2ccccc2-c2ccc(N(c3ccc(-c4cccc5ccccc45)cc3)c3ccc(-c4cccc5c4sc4ccccc45)cc3)cc21 ; c1ccc2c(c1)-c1ccccc1C21c2ccccc2-c2ccc(N(c3ccc(-c4ccc5ccccc5c4)cc3)c3ccc(-c4cccc5c4sc4ccccc45)cc3)cc21]

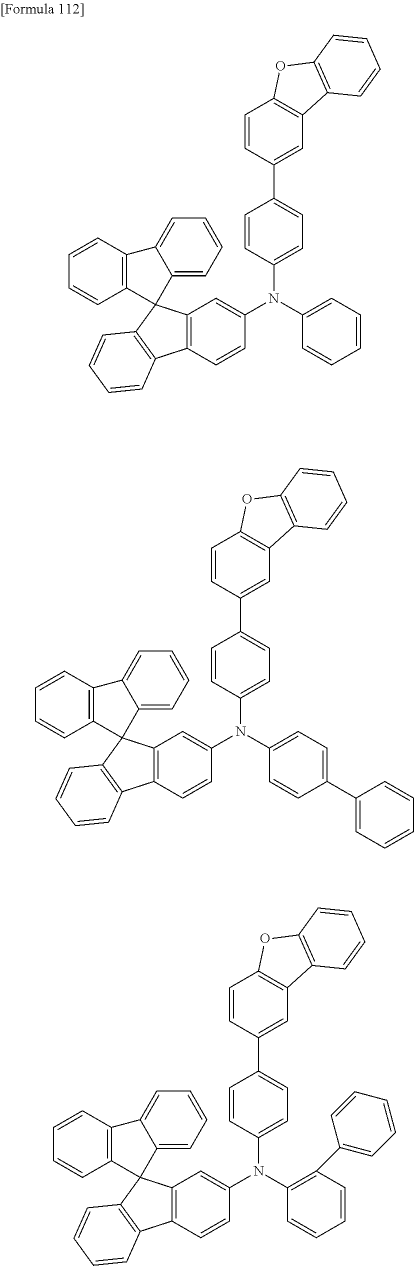 [SMILES: c1ccc(N(c2ccc(-c3ccc4oc5ccccc5c4c3)cc2)c2ccc3c(c2)C2(c4ccccc4-c4ccccc42)c2ccccc2-3)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccccc2N(c2ccc(-c3ccc4oc5ccccc5c4c3)cc2)c2ccc3c(c2)C2(c4ccccc4-c4ccccc42)c2ccccc2-3)cc1]

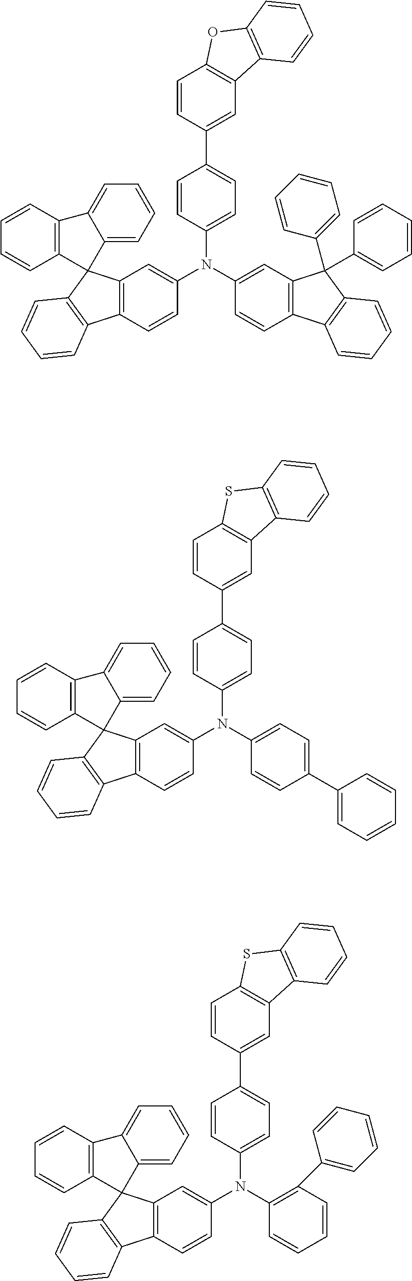 [SMILES: c1ccc(C2(c3ccccc3)c3ccccc3-c3ccc(N(c4ccc(-c5ccc6oc7ccccc7c6c5)cc4)c4ccc5c(c4)C4(c6ccccc6-c6ccccc64)c4ccccc4-5)cc32)cc1 ; c1ccc(-c2ccc(N(c3ccc(-c4ccc5sc6ccccc6c5c4)cc3)c3ccc4c(c3)C3(c5ccccc5-c5ccccc53)c3ccccc3-4)cc2)cc1 ; c1ccc(-c2ccccc2N(c2ccc(-c3ccc4sc5ccccc5c4c3)cc2)c2ccc3c(c2)C2(c4ccccc4-c4ccccc42)c2ccccc2-3)cc1]

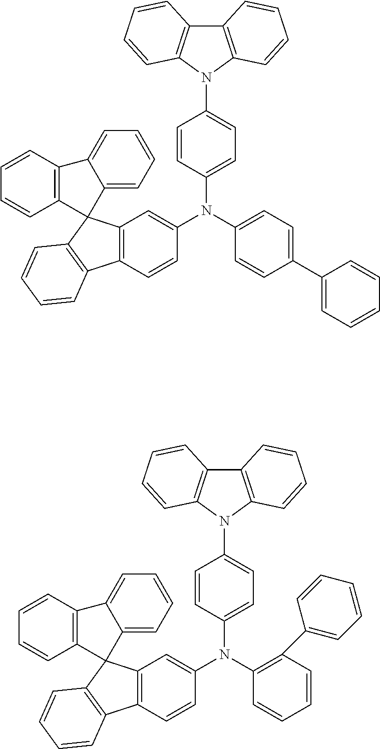 [SMILES: c1ccc(-c2ccc(N(c3ccc(-n4c5ccccc5c5ccccc54)cc3)c3ccc4c(c3)C3(c5ccccc5-c5ccccc53)c3ccccc3-4)cc2)cc1 ; c1ccc(-c2ccccc2N(c2ccc(-n3c4ccccc4c4ccccc43)cc2)c2ccc3c(c2)C2(c4ccccc4-c4ccccc42)c2ccccc2-3)cc1]

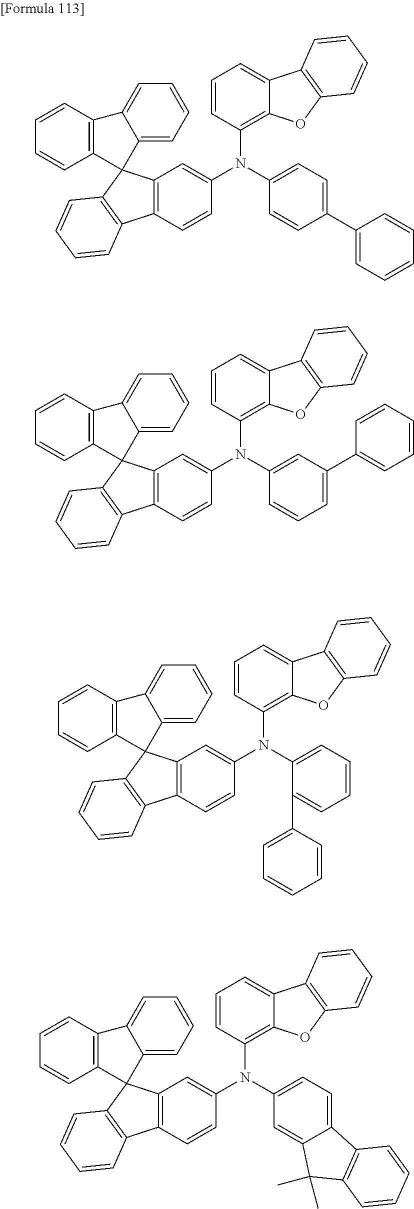 [SMILES: c1ccc(-c2ccc(N(c3ccc4c(c3)C3(c5ccccc5-c5ccccc53)c3ccccc3-4)c3cccc4c3oc3ccccc34)cc2)cc1 ; c1ccc(-c2cccc(N(c3ccc4c(c3)C3(c5ccccc5-c5ccccc53)c3ccccc3-4)c3cccc4c3oc3ccccc34)c2)cc1 ; c1ccc(-c2ccccc2N(c2ccc3c(c2)C2(c4ccccc4-c4ccccc42)c2ccccc2-3)c2cccc3c2oc2ccccc23)cc1 ; CC1(C)c2ccccc2-c2ccc(N(c3ccc4c(c3)C3(c5ccccc5-c5ccccc53)c3ccccc3-4)c3cccc4c3oc3ccccc34)cc21]

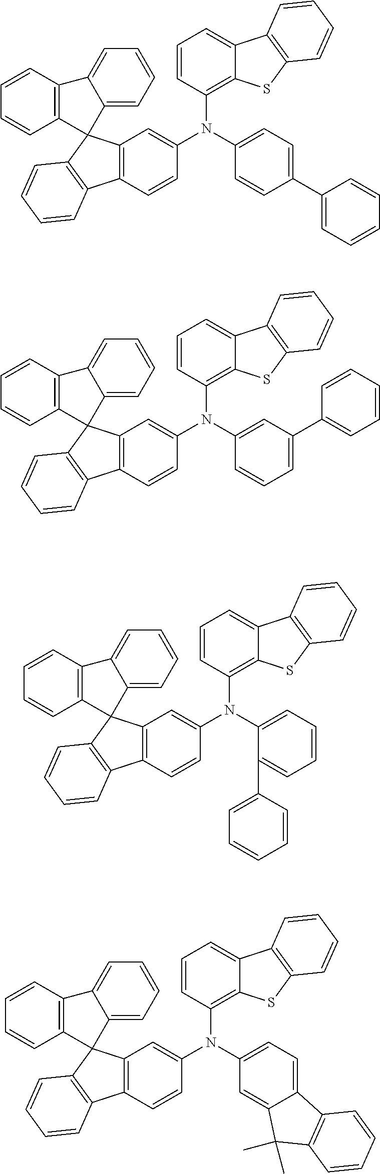 [SMILES: c1ccc(-c2ccc(N(c3ccc4c(c3)C3(c5ccccc5-c5ccccc53)c3ccccc3-4)c3cccc4c3sc3ccccc34)cc2)cc1 ; c1ccc(-c2cccc(N(c3ccc4c(c3)C3(c5ccccc5-c5ccccc53)c3ccccc3-4)c3cccc4c3sc3ccccc34)c2)cc1 ; c1ccc(-c2ccccc2N(c2ccc3c(c2)C2(c4ccccc4-c4ccccc42)c2ccccc2-3)c2cccc3c2sc2ccccc23)cc1 ; CC1(C)c2ccccc2-c2ccc(N(c3ccc4c(c3)C3(c5ccccc5-c5ccccc53)c3ccccc3-4)c3cccc4c3sc3ccccc34)cc21]

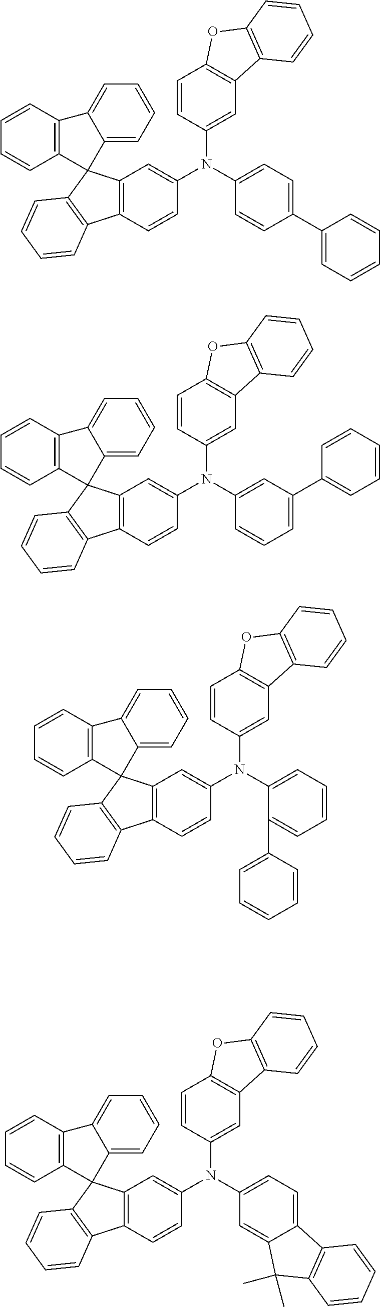 [SMILES: c1ccc(-c2ccc(N(c3ccc4c(c3)C3(c5ccccc5-c5ccccc53)c3ccccc3-4)c3ccc4oc5ccccc5c4c3)cc2)cc1 ; c1ccc(-c2cccc(N(c3ccc4c(c3)C3(c5ccccc5-c5ccccc53)c3ccccc3-4)c3ccc4oc5ccccc5c4c3)c2)cc1 ; c1ccc(-c2ccccc2N(c2ccc3c(c2)C2(c4ccccc4-c4ccccc42)c2ccccc2-3)c2ccc3oc4ccccc4c3c2)cc1 ; CC1(C)c2ccccc2-c2ccc(N(c3ccc4c(c3)C3(c5ccccc5-c5ccccc53)c3ccccc3-4)c3ccc4oc5ccccc5c4c3)cc21]

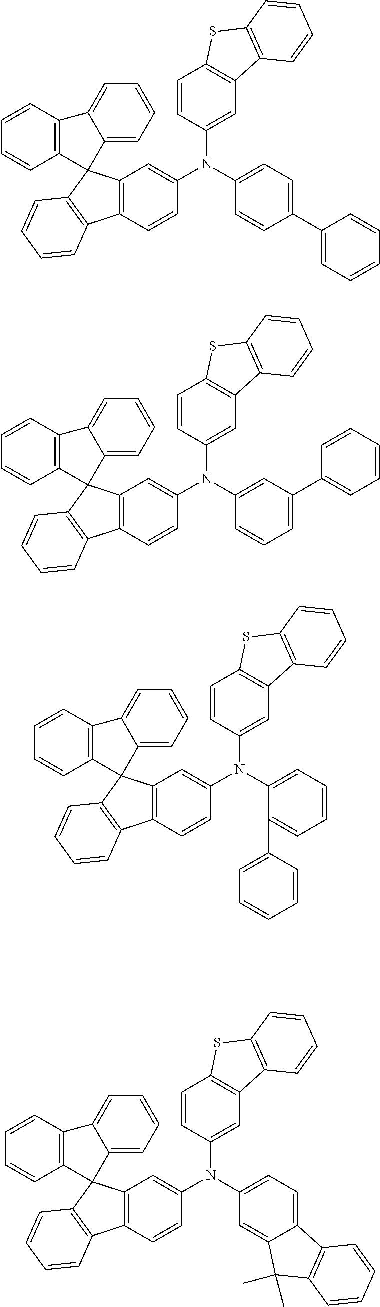 [SMILES: c1ccc(-c2ccc(N(c3ccc4c(c3)C3(c5ccccc5-c5ccccc53)c3ccccc3-4)c3ccc4sc5ccccc5c4c3)cc2)cc1 ; c1ccc(-c2cccc(N(c3ccc4c(c3)C3(c5ccccc5-c5ccccc53)c3ccccc3-4)c3ccc4sc5ccccc5c4c3)c2)cc1 ; c1ccc(-c2ccccc2N(c2ccc3c(c2)C2(c4ccccc4-c4ccccc42)c2ccccc2-3)c2ccc3sc4ccccc4c3c2)cc1 ; CC1(C)c2ccccc2-c2ccc(N(c3ccc4c(c3)C3(c5ccccc5-c5ccccc53)c3ccccc3-4)c3ccc4sc5ccccc5c4c3)cc21]

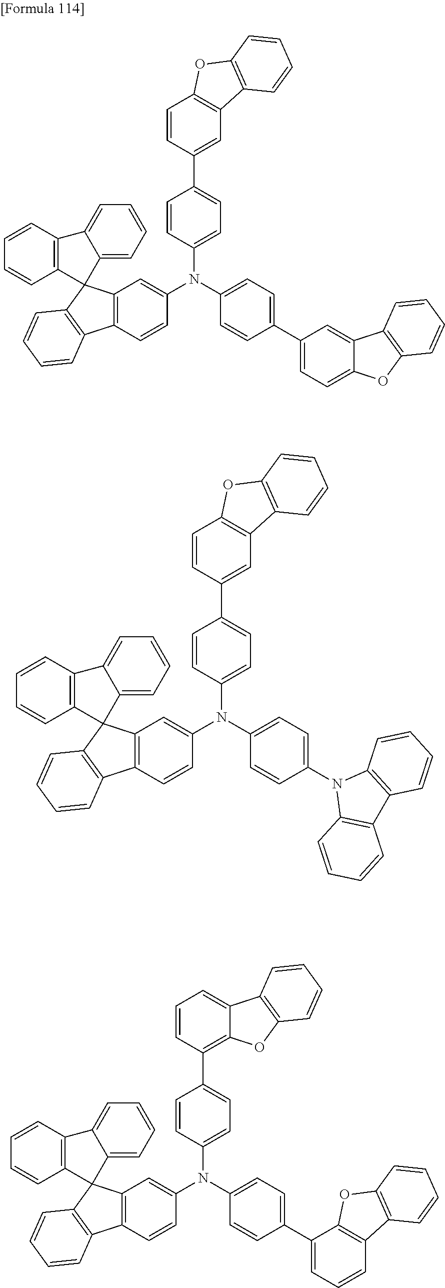 [SMILES: c1ccc2c(c1)-c1ccccc1C21c2ccccc2-c2ccc(N(c3ccc(-c4ccc5oc6ccccc6c5c4)cc3)c3ccc(-c4ccc5oc6ccccc6c5c4)cc3)cc21 ; c1ccc2c(c1)-c1ccccc1C21c2ccccc2-c2ccc(N(c3ccc(-c4ccc5oc6ccccc6c5c4)cc3)c3ccc(-n4c5ccccc5c5ccccc54)cc3)cc21 ; c1ccc2c(c1)-c1ccccc1C21c2ccccc2-c2ccc(N(c3ccc(-c4cccc5c4oc4ccccc45)cc3)c3ccc(-c4cccc5c4oc4ccccc45)cc3)cc21]

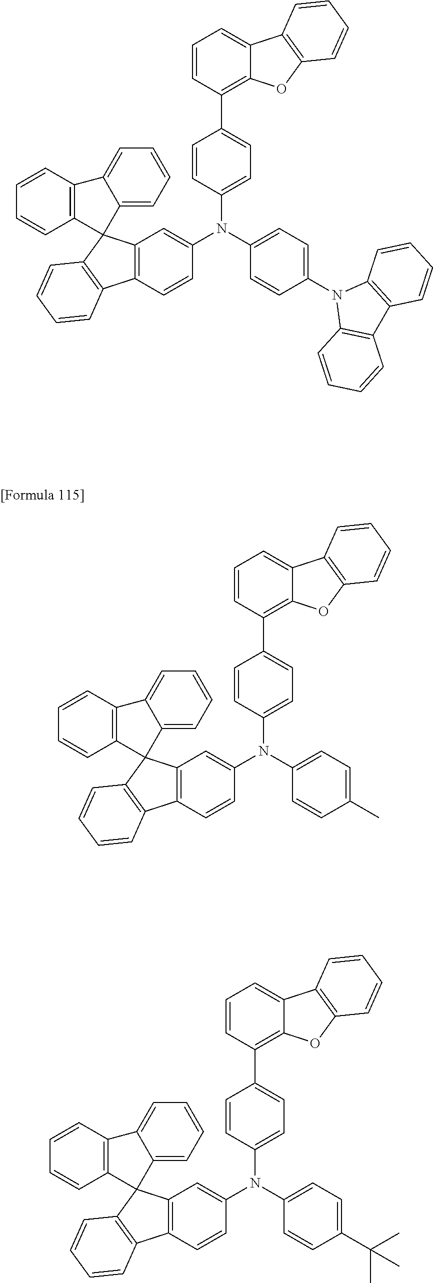 [SMILES: c1ccc2c(c1)-c1ccccc1C21c2ccccc2-c2ccc(N(c3ccc(-c4cccc5c4oc4ccccc45)cc3)c3ccc(-n4c5ccccc5c5ccccc54)cc3)cc21 ; Cc1ccc(N(c2ccc(-c3cccc4c3oc3ccccc34)cc2)c2ccc3c(c2)C2(c4ccccc4-c4ccccc42)c2ccccc2-3)cc1 ; CC(C)(C)c1ccc(N(c2ccc(-c3cccc4c3oc3ccccc34)cc2)c2ccc3c(c2)C2(c4ccccc4-c4ccccc42)c2ccccc2-3)cc1]

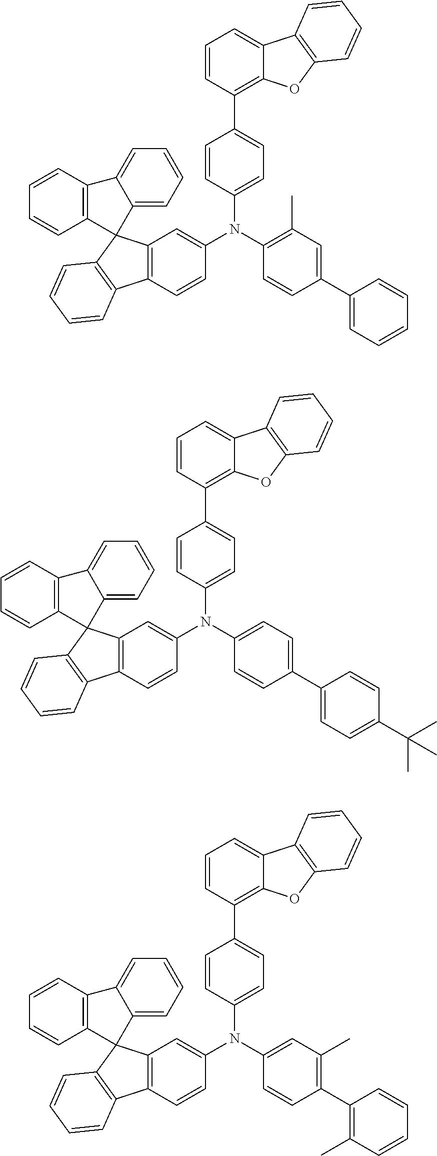 [SMILES: Cc1cc(-c2ccccc2)ccc1N(c1ccc(-c2cccc3c2oc2ccccc23)cc1)c1ccc2c(c1)C1(c3ccccc3-c3ccccc31)c1ccccc1-2 ; CC(C)(C)c1ccc(-c2ccc(N(c3ccc(-c4cccc5c4oc4ccccc45)cc3)c3ccc4c(c3)C3(c5ccccc5-c5ccccc53)c3ccccc3-4)cc2)cc1 ; Cc1ccccc1-c1ccc(N(c2ccc(-c3cccc4c3oc3ccccc34)cc2)c2ccc3c(c2)C2(c4ccccc4-c4ccccc42)c2ccccc2-3)cc1C]

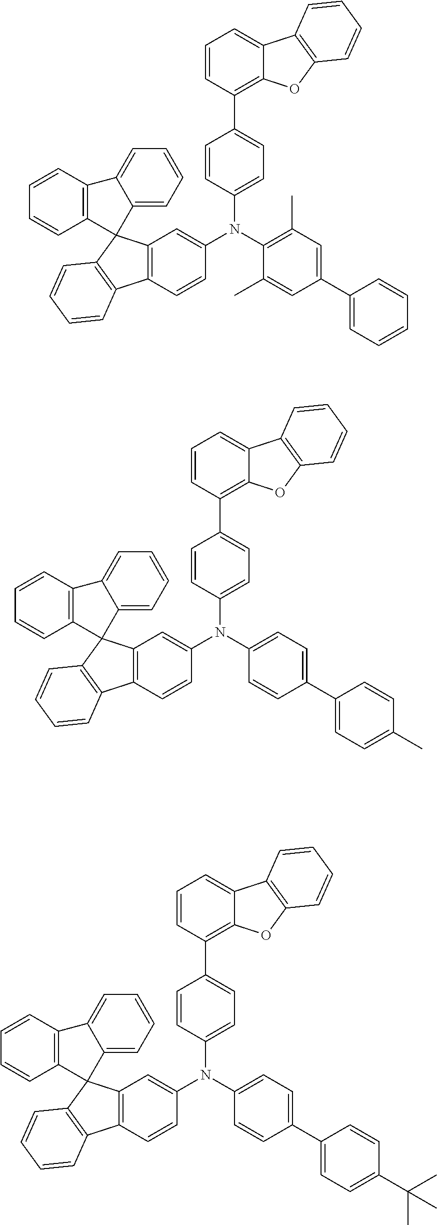 [SMILES: Cc1cc(-c2ccccc2)cc(C)c1N(c1ccc(-c2cccc3c2oc2ccccc23)cc1)c1ccc2c(c1)C1(c3ccccc3-c3ccccc31)c1ccccc1-2 ; Cc1ccc(-c2ccc(N(c3ccc(-c4cccc5c4oc4ccccc45)cc3)c3ccc4c(c3)C3(c5ccccc5-c5ccccc53)c3ccccc3-4)cc2)cc1 ; CC(C)(C)c1ccc(-c2ccc(N(c3ccc(-c4cccc5c4oc4ccccc45)cc3)c3ccc4c(c3)C3(c5ccccc5-c5ccccc53)c3ccccc3-4)cc2)cc1]

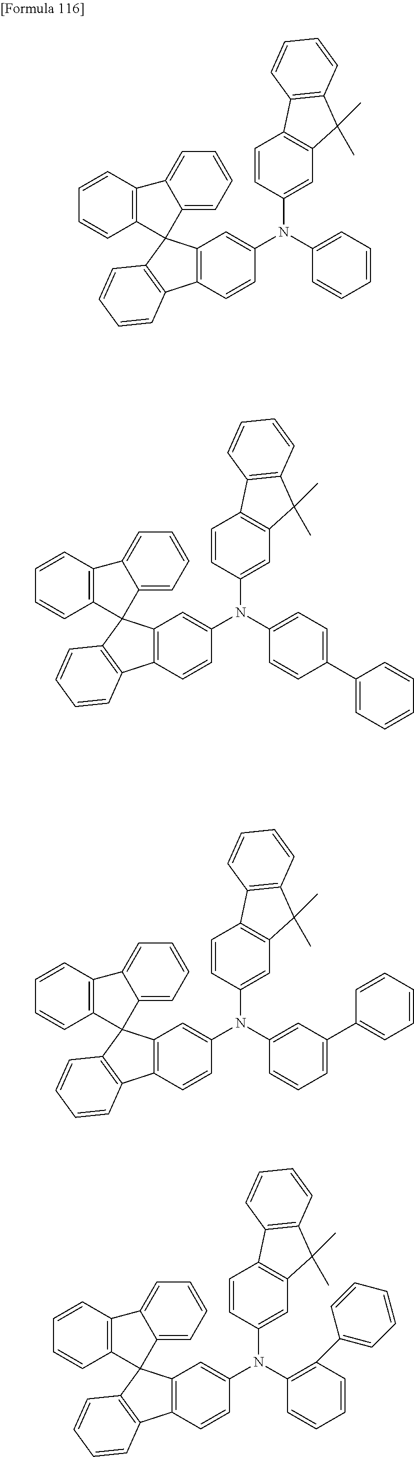 [SMILES: CC1(C)c2ccccc2-c2ccc(N(c3ccccc3)c3ccc4c(c3)C3(c5ccccc5-c5ccccc53)c3ccccc3-4)cc21 ; CC1(C)c2ccccc2-c2ccc(N(c3ccc(-c4ccccc4)cc3)c3ccc4c(c3)C3(c5ccccc5-c5ccccc53)c3ccccc3-4)cc21 ; CC1(C)c2ccccc2-c2ccc(N(c3cccc(-c4ccccc4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

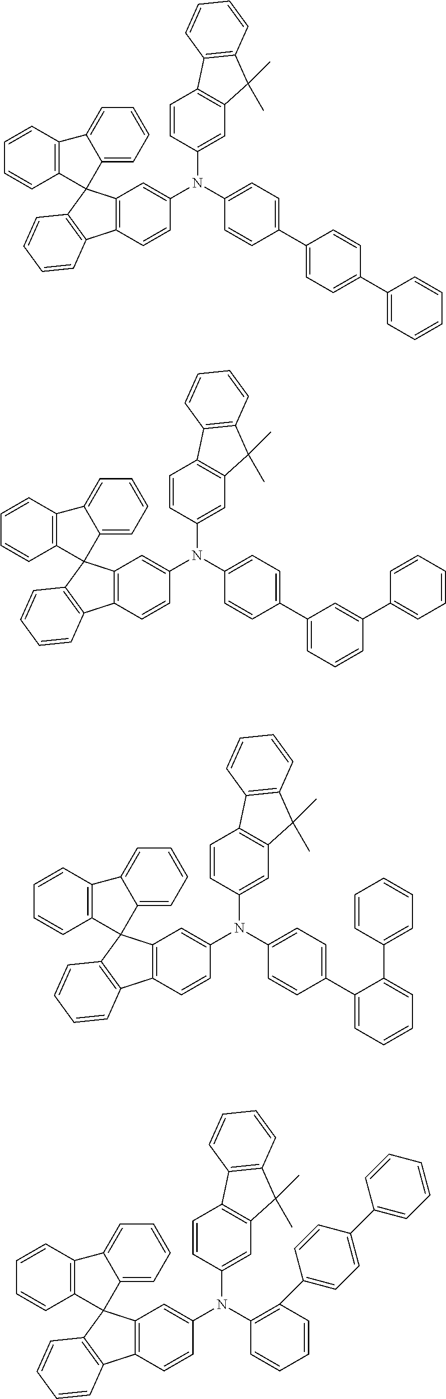 [SMILES: CC1(C)c2ccccc2-c2ccc(N(c3ccc(-c4ccc(-c5ccccc5)cc4)cc3)c3ccc4c(c3)C3(c5ccccc5-c5ccccc53)c3ccccc3-4)cc21 ; CC1(C)c2ccccc2-c2ccc(N(c3ccc(-c4cccc(-c5ccccc5)c4)cc3)c3ccc4c(c3)C3(c5ccccc5-c5ccccc53)c3ccccc3-4)cc21 ; CC1(C)c2ccccc2-c2ccc(N(c3ccc(-c4ccccc4-c4ccccc4)cc3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccc(-c4ccccc4)cc3)cc21]

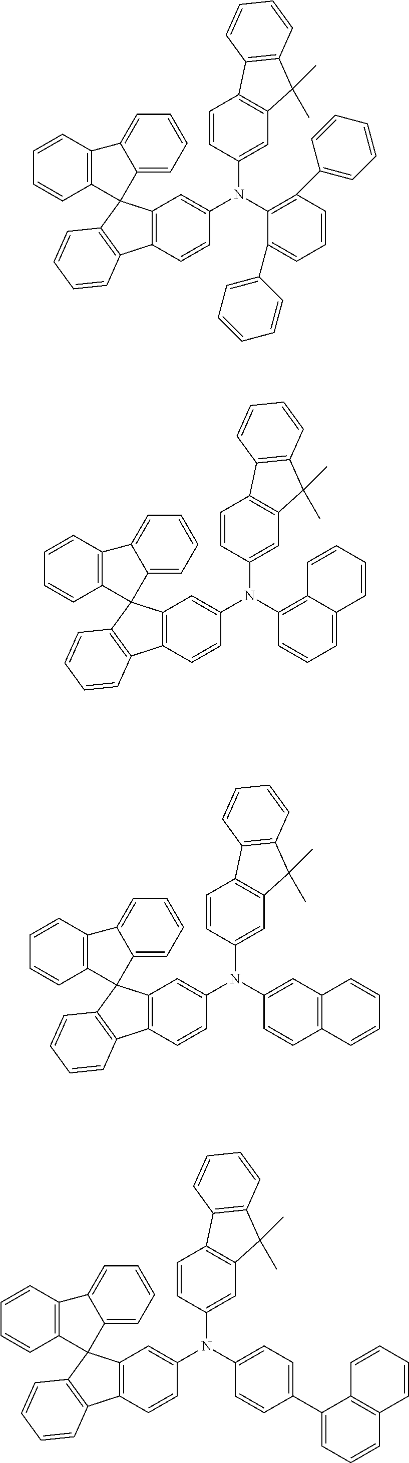 [SMILES: CC1(C)c2ccccc2-c2ccc(N(c3ccc4c(c3)C3(c5ccccc5-c5ccccc53)c3ccccc3-4)c3c(-c4ccccc4)cccc3-c3ccccc3)cc21 ; CC1(C)c2ccccc2-c2ccc(N(c3ccc4c(c3)C3(c5ccccc5-c5ccccc53)c3ccccc3-4)c3cccc4ccccc34)cc21 ; CC1(C)c2ccccc2-c2ccc(N(c3ccc4c(c3)C3(c5ccccc5-c5ccccc53)c3ccccc3-4)c3ccc4ccccc4c3)cc21 ; CC1(C)c2ccccc2-c2ccc(N(c3ccc(-c4cccc5ccccc45)cc3)c3ccc4c(c3)C3(c5ccccc5-c5ccccc53)c3ccccc3-4)cc21]

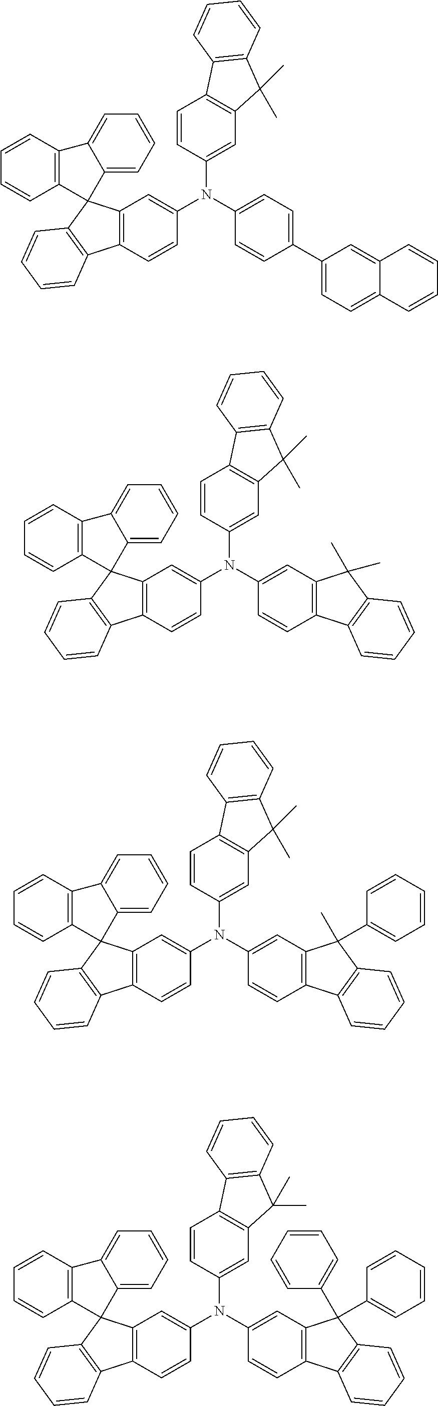 [SMILES: CC1(C)c2ccccc2-c2ccc(N(c3ccc(-c4ccc5ccccc5c4)cc3)c3ccc4c(c3)C3(c5ccccc5-c5ccccc53)c3ccccc3-4)cc21 ; CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(c3ccccc3)c3ccccc3-4)c3ccc4c(c3)C3(c5ccccc5-c5ccccc53)c3ccccc3-4)cc21 ; CC1(C)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21]

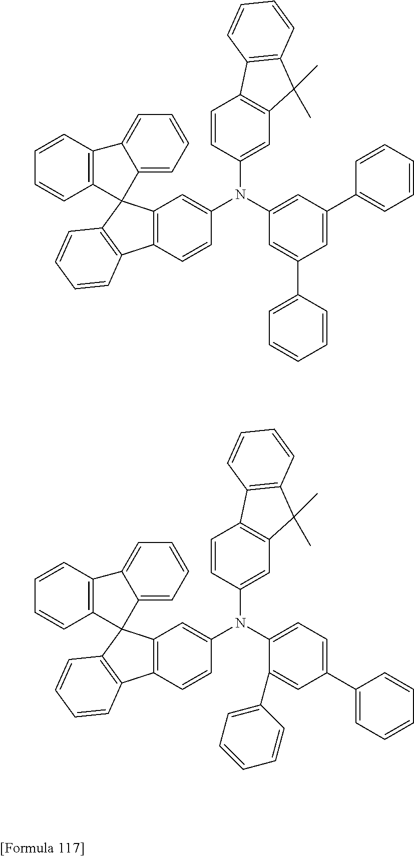 [SMILES: CC1(C)c2ccccc2-c2ccc(N(c3cc(-c4ccccc4)cc(-c4ccccc4)c3)c3ccc4c(c3)C3(c5ccccc5-c5ccccc53)c3ccccc3-4)cc21 ; CC1(C)c2ccccc2-c2ccc(N(c3ccc4c(c3)C3(c5ccccc5-c5ccccc53)c3ccccc3-4)c3ccc(-c4ccccc4)cc3-c3ccccc3)cc21]

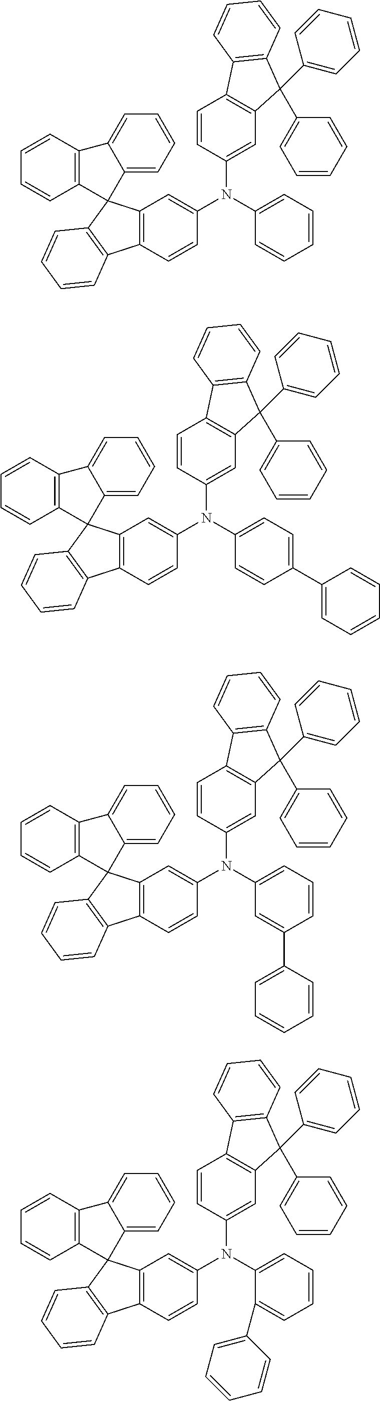

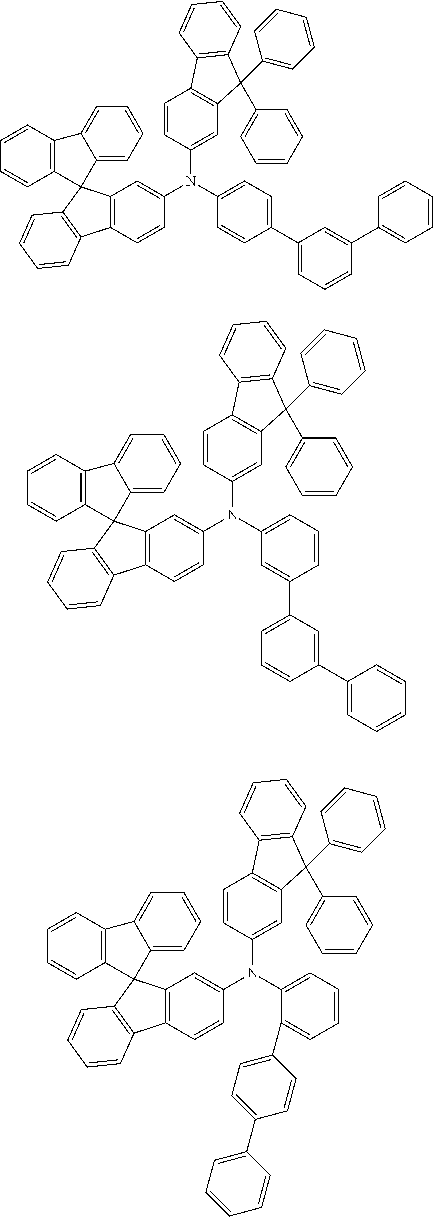

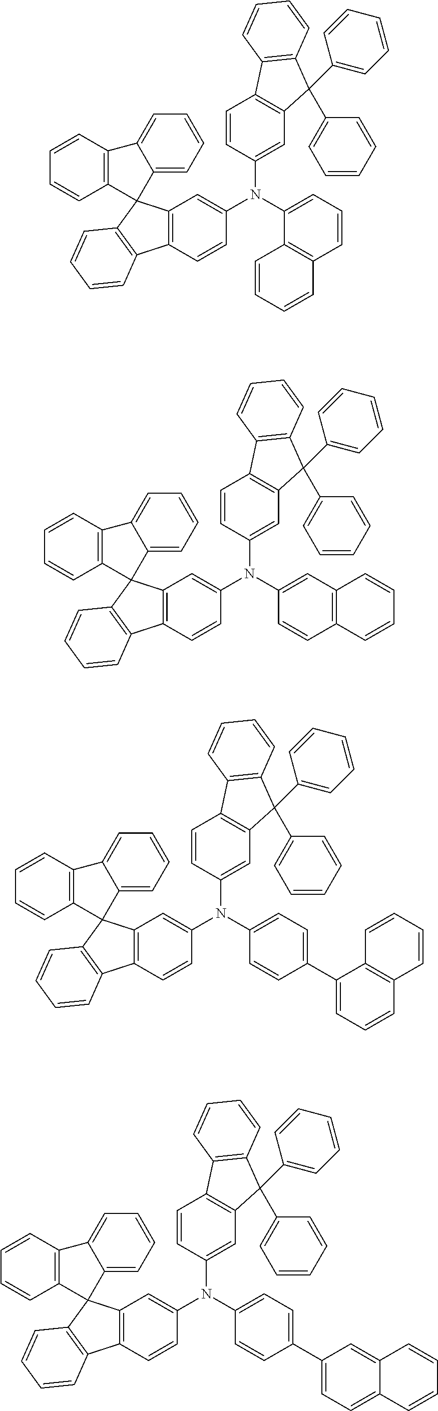 [SMILES: c1ccc(C2(c3ccccc3)c3ccccc3-c3ccc(N(c4ccc5c(c4)C4(c6ccccc6-c6ccccc64)c4ccccc4-5)c4cccc5ccccc45)cc32)cc1 ; c1ccc(C2(c3ccccc3)c3ccccc3-c3ccc(N(c4ccc5c(c4)C4(c6ccccc6-c6ccccc64)c4ccccc4-5)c4ccc5ccccc5c4)cc32)cc1 ; c1ccc(C2(c3ccccc3)c3ccccc3-c3ccc(N(c4ccc(-c5cccc6ccccc56)cc4)c4ccc5c(c4)C4(c6ccccc6-c6ccccc64)c4ccccc4-5)cc32)cc1 ; c1ccc(C2(c3ccccc3)c3ccccc3-c3ccc(N(c4ccc(-c5ccc6ccccc6c5)cc4)c4ccc5c(c4)C4(c6ccccc6-c6ccccc64)c4ccccc4-5)cc32)cc1]

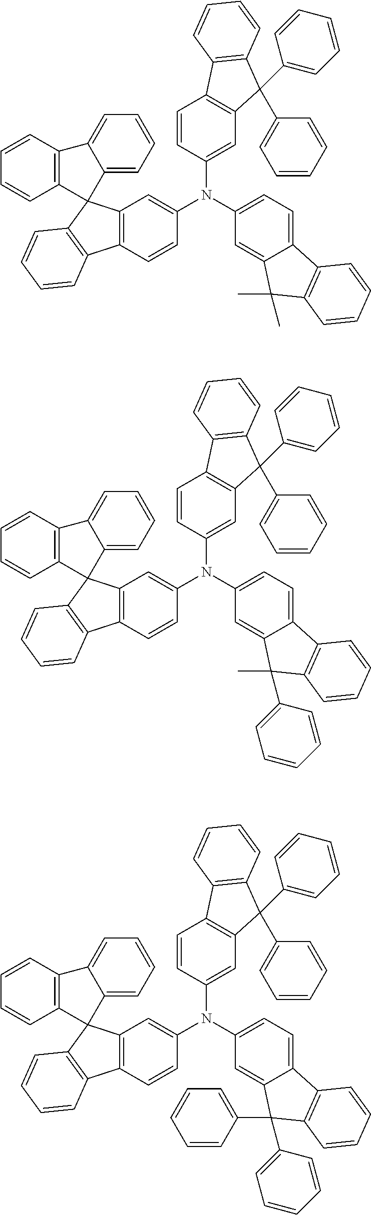 [SMILES: CC1(C)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21 ; CC1(c2ccccc2)c2ccccc2-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc21 ; c1ccc(C2(c3ccccc3)c3ccccc3-c3ccc(N(c4ccc5c(c4)C(c4ccccc4)(c4ccccc4)c4ccccc4-5)c4ccc5c(c4)C4(c6ccccc6-c6ccccc64)c4ccccc4-5)cc32)cc1]

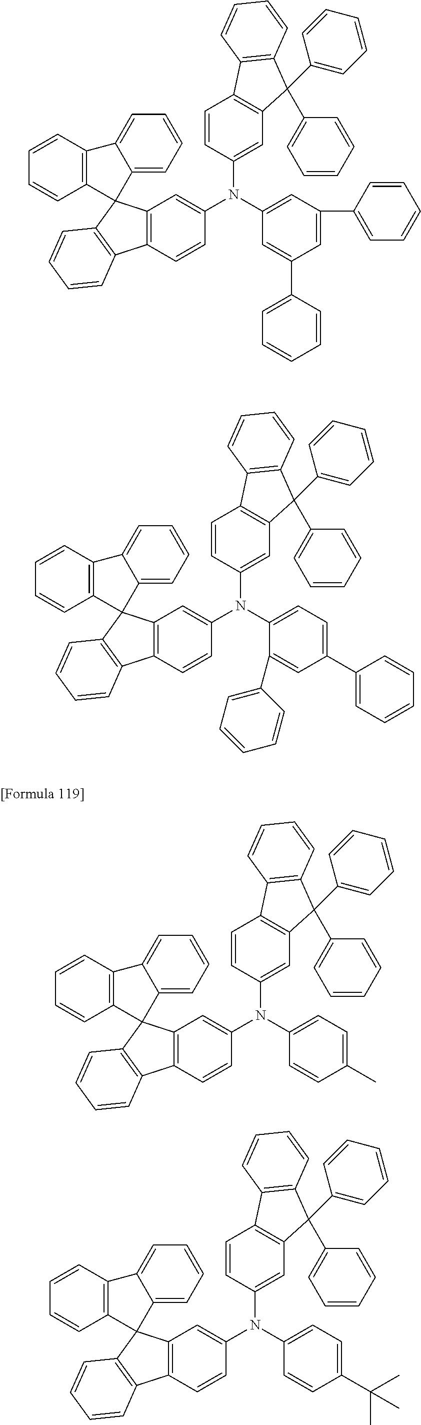 [SMILES: c1ccc(-c2cc(-c3ccccc3)cc(N(c3ccc4c(c3)C(c3ccccc3)(c3ccccc3)c3ccccc3-4)c3ccc4c(c3)C3(c5ccccc5-c5ccccc53)c3ccccc3-4)c2)cc1 ; c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)c(-c3ccccc3)c2)cc1 ; Cc1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1 ; CC(C)(C)c1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1]

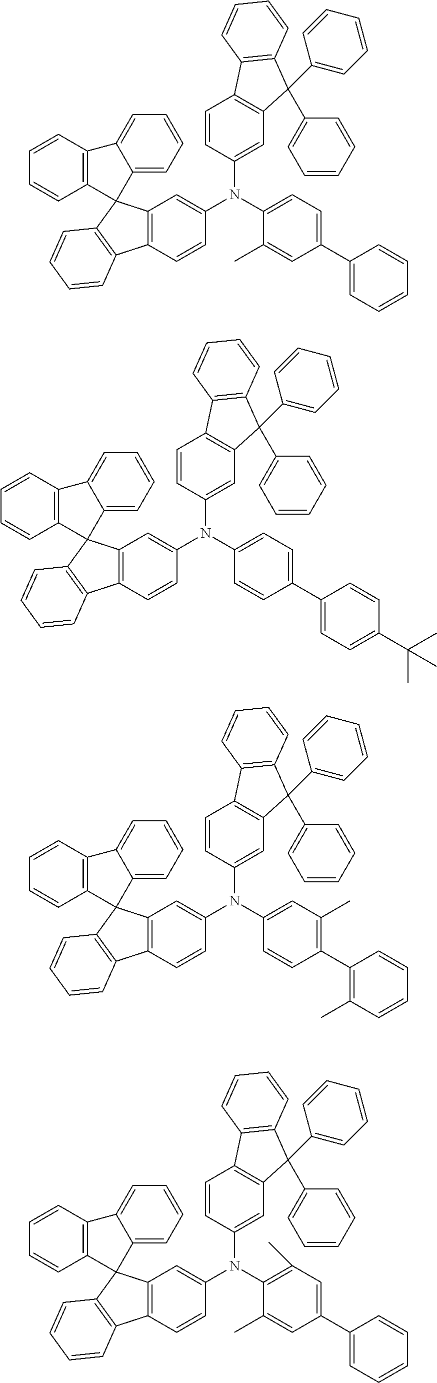 [SMILES: Cc1cc(-c2ccccc2)ccc1N(c1ccc2c(c1)C(c1ccccc1)(c1ccccc1)c1ccccc1-2)c1ccc2c(c1)C1(c3ccccc3-c3ccccc31)c1ccccc1-2 ; CC(C)(C)c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1 ; Cc1ccccc1-c1ccc(N(c2ccc3c(c2)C(c2ccccc2)(c2ccccc2)c2ccccc2-3)c2ccc3c(c2)C2(c4ccccc4-c4ccccc42)c2ccccc2-3)cc1C ; Cc1cc(-c2ccccc2)cc(C)c1N(c1ccc2c(c1)C(c1ccccc1)(c1ccccc1)c1ccccc1-2)c1ccc2c(c1)C1(c3ccccc3-c3ccccc31)c1ccccc1-2]

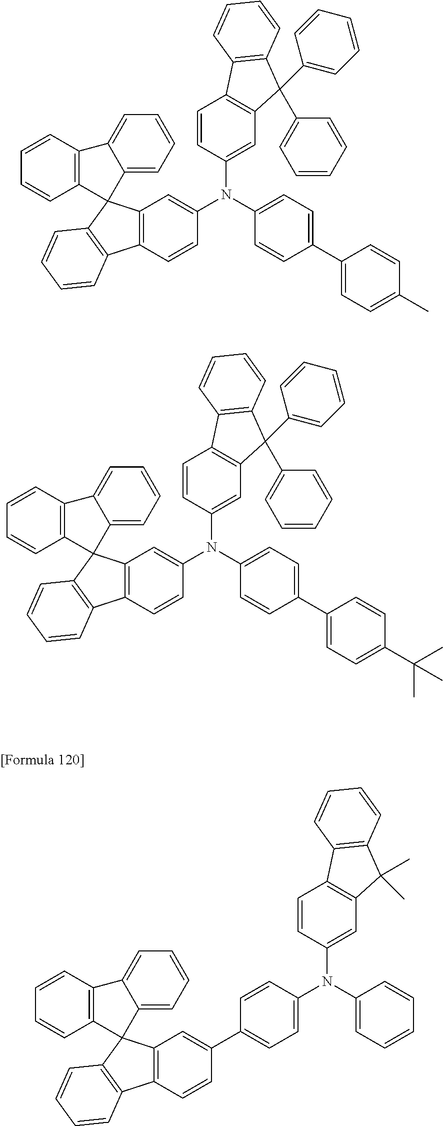 [SMILES: Cc1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1 ; CC(C)(C)c1ccc(-c2ccc(N(c3ccc4c(c3)C(c3ccccc3)(c3ccccc3)c3ccccc3-4)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c(c4)C4(c6ccccc6-c6ccccc64)c4ccccc4-5)cc3)cc21]

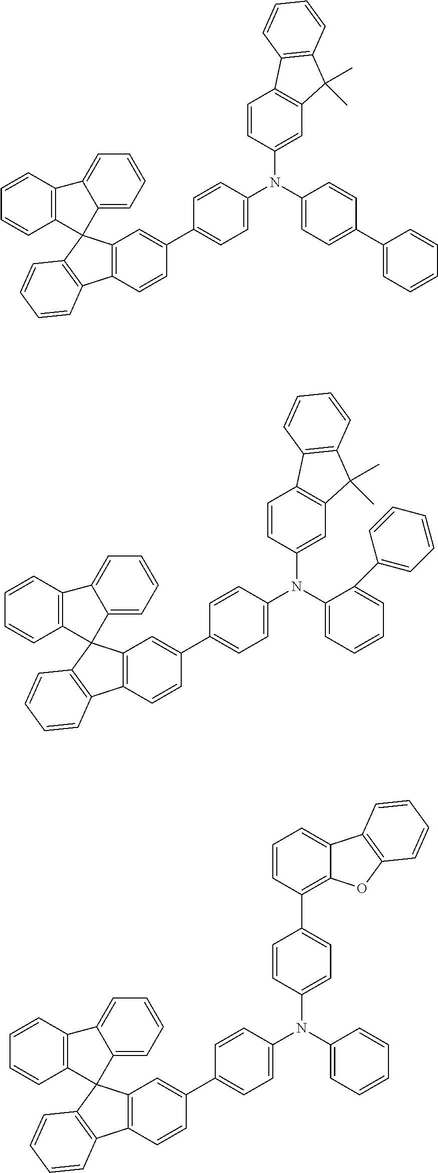 [SMILES: CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21 ; CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21 ; c1ccc(N(c2ccc(-c3ccc4c(c3)C3(c5ccccc5-c5ccccc53)c3ccccc3-4)cc2)c2ccc(-c3cccc4c3oc3ccccc34)cc2)cc1]

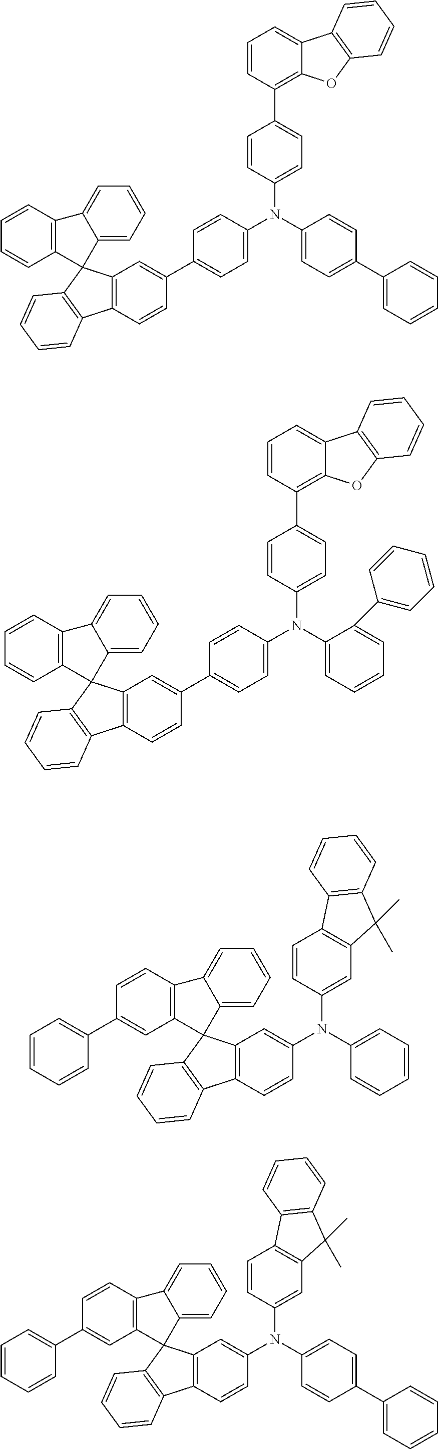 [SMILES: c1ccc(-c2ccc(N(c3ccc(-c4ccc5c(c4)C4(c6ccccc6-c6ccccc64)c4ccccc4-5)cc3)c3ccc(-c4cccc5c4oc4ccccc45)cc3)cc2)cc1 ; c1ccc(-c2ccccc2N(c2ccc(-c3ccc4c(c3)C3(c5ccccc5-c5ccccc53)c3ccccc3-4)cc2)c2ccc(-c3cccc4c3oc3ccccc34)cc2)cc1 ; CC1(C)c2ccccc2-c2ccc(N(c3ccccc3)c3ccc4c(c3)C3(c5ccccc5-c5ccc(-c6ccccc6)cc53)c3ccccc3-4)cc21 ; CC1(C)c2ccccc2-c2ccc(N(c3ccc(-c4ccccc4)cc3)c3ccc4c(c3)C3(c5ccccc5-c5ccc(-c6ccccc6)cc53)c3ccccc3-4)cc21]

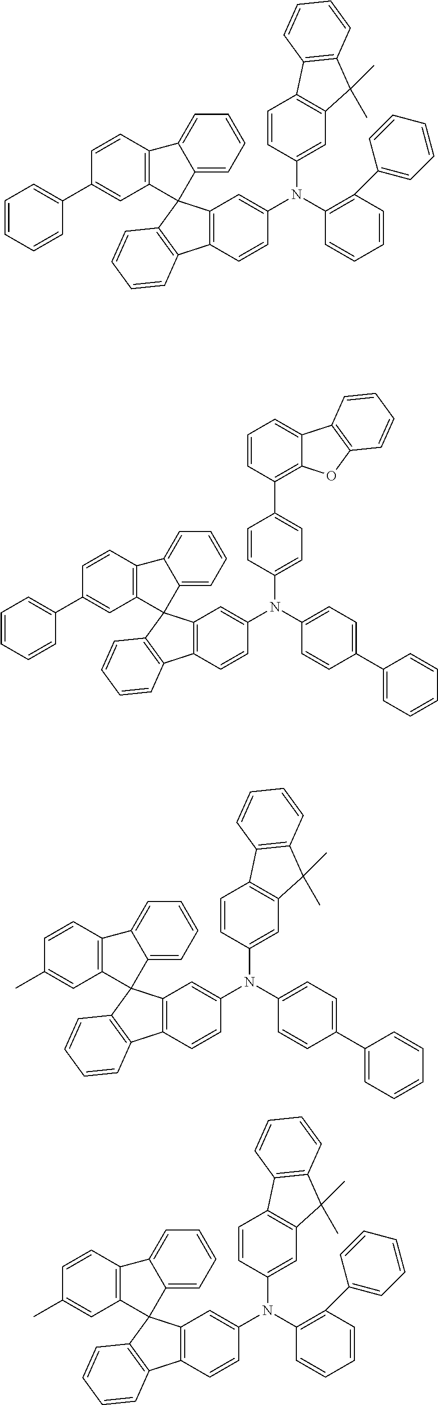 [SMILES: CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21 ; c1ccc(-c2ccc(N(c3ccc(-c4cccc5c4oc4ccccc45)cc3)c3ccc4c(c3)C3(c5ccccc5-c5ccc(-c6ccccc6)cc53)c3ccccc3-4)cc2)cc1 ; Cc1ccc2c(c1)C1(c3ccccc3-2)c2ccccc2-c2ccc(N(c3ccc(-c4ccccc4)cc3)c3ccc4c(c3)C(C)(C)c3ccccc3-4)cc21 ; Cc1ccc2c(c1)C1(c3ccccc3-2)c2ccccc2-c2ccc(N(c3ccc4c(c3)C(C)(C)c3ccccc3-4)c3ccccc3-c3ccccc3)cc21]

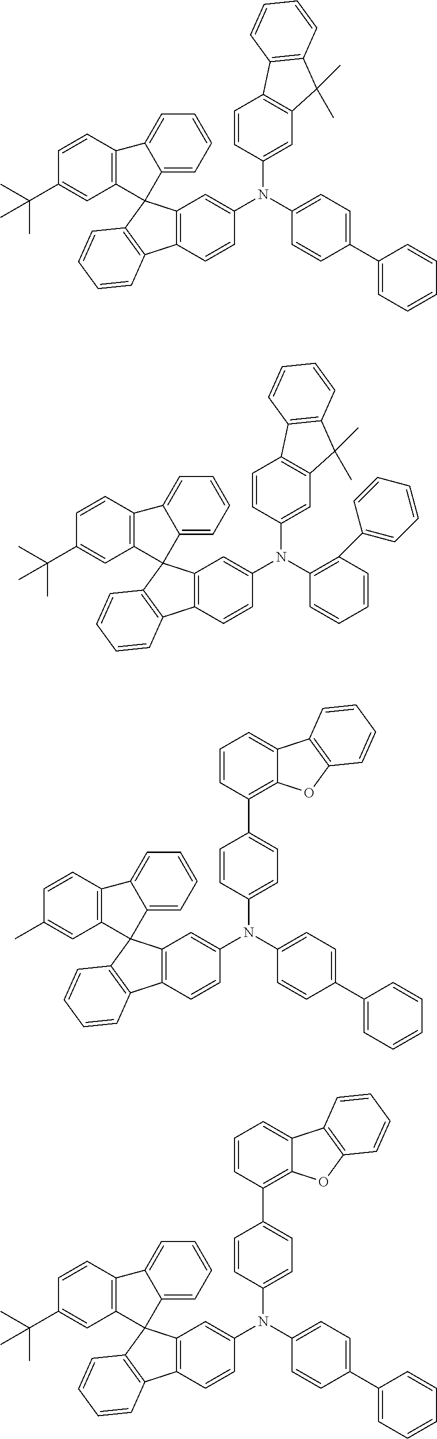 [SMILES: CC(C)(C)c1ccc2c(c1)C1(c3ccccc3-c3ccc(N(c4ccc(-c5ccccc5)cc4)c4ccc5c(c4)C(C)(C)c4ccccc4-5)cc31)c1ccccc1-2 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1ccccc1-2 ; Cc1ccc2c(c1)C1(c3ccccc3-2)c2ccccc2-c2ccc(N(c3ccc(-c4ccccc4)cc3)c3ccc(-c4cccc5c4oc4ccccc45)cc3)cc21 ; CC(C)(C)c1ccc2c(c1)C1(c3ccccc3-c3ccc(N(c4ccc(-c5ccccc5)cc4)c4ccc(-c5cccc6c5oc5ccccc56)cc4)cc31)c1ccccc1-2]

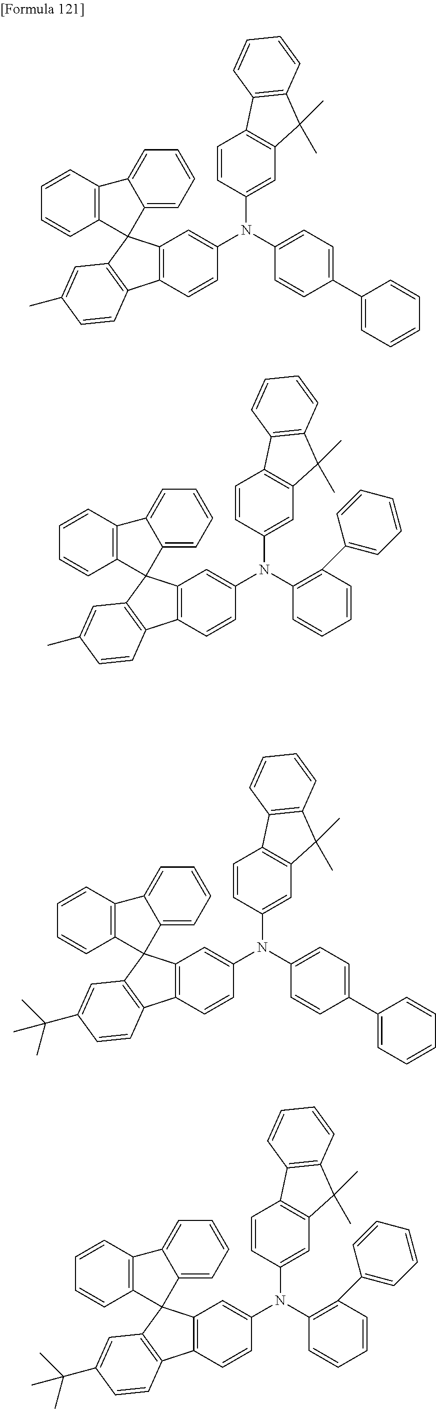 [SMILES: Cc1ccc2c(c1)C1(c3ccccc3-c3ccccc31)c1cc(N(c3ccc(-c4ccccc4)cc3)c3ccc4c(c3)C(C)(C)c3ccccc3-4)ccc1-2 ; Cc1ccc2c(c1)C1(c3ccccc3-c3ccccc31)c1cc(N(c3ccc4c(c3)C(C)(C)c3ccccc3-4)c3ccccc3-c3ccccc3)ccc1-2 ; CC(C)(C)c1ccc2c(c1)C1(c3ccccc3-c3ccccc31)c1cc(N(c3ccc(-c4ccccc4)cc3)c3ccc4c(c3)C(C)(C)c3ccccc3-4)ccc1-2 ; CC(C)(C)c1ccc2c(c1)C1(c3ccccc3-c3ccccc31)c1cc(N(c3ccc4c(c3)C(C)(C)c3ccccc3-4)c3ccccc3-c3ccccc3)ccc1-2]

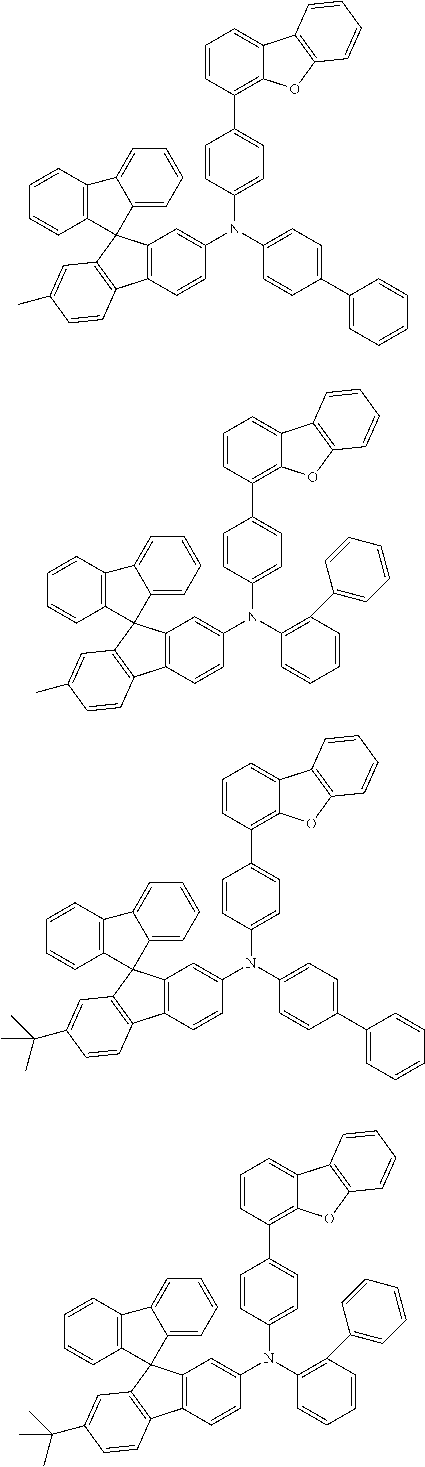 [SMILES: Cc1ccc2c(c1)C1(c3ccccc3-c3ccccc31)c1cc(N(c3ccc(-c4ccccc4)cc3)c3ccc(-c4cccc5c4oc4ccccc45)cc3)ccc1-2 ; Cc1ccc2c(c1)C1(c3ccccc3-c3ccccc31)c1cc(N(c3ccc(-c4cccc5c4oc4ccccc45)cc3)c3ccccc3-c3ccccc3)ccc1-2 ; CC(C)(C)c1ccc2c(c1)C1(c3ccccc3-c3ccccc31)c1cc(N(c3ccc(-c4ccccc4)cc3)c3ccc(-c4cccc5c4oc4ccccc45)cc3)ccc1-2 ; CC(C)(C)c1ccc2c(c1)C1(c3ccccc3-c3ccccc31)c1cc(N(c3ccc(-c4cccc5c4oc4ccccc45)cc3)c3ccccc3-c3ccccc3)ccc1-2]

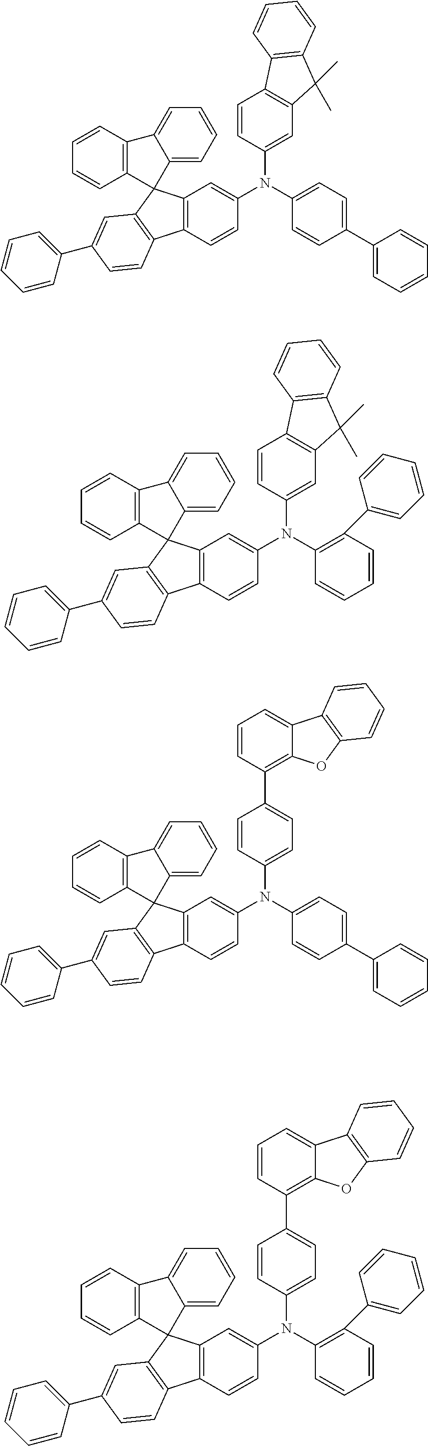 [SMILES: CC1(C)c2ccccc2-c2ccc(N(c3ccc(-c4ccccc4)cc3)c3ccc4c(c3)C3(c5ccccc5-c5ccccc53)c3cc(-c5ccccc5)ccc3-4)cc21 ; CC1(C)c2ccccc2-c2ccc(N(c3ccc4c(c3)C3(c5ccccc5-c5ccccc53)c3cc(-c5ccccc5)ccc3-4)c3ccccc3-c3ccccc3)cc21 ; c1ccc(-c2ccc(N(c3ccc(-c4cccc5c4oc4ccccc45)cc3)c3ccc4c(c3)C3(c5ccccc5-c5ccccc53)c3cc(-c5ccccc5)ccc3-4)cc2)cc1 ; c1ccc(-c2ccc3c(c2)C2(c4ccccc4-c4ccccc42)c2cc(N(c4ccc(-c5cccc6c5oc5ccccc56)cc4)c4ccccc4-c4ccccc4)ccc2-3)cc1]

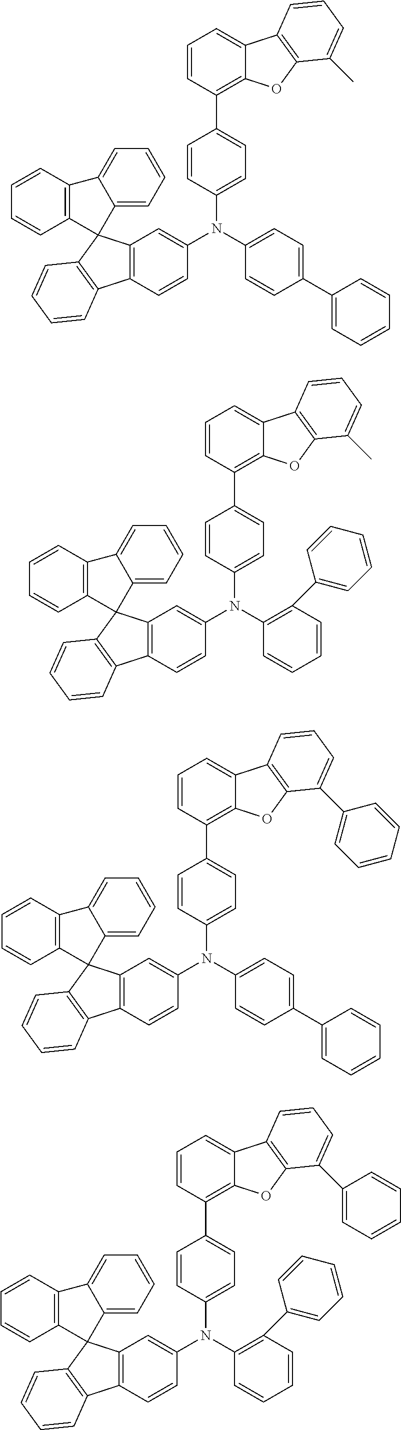

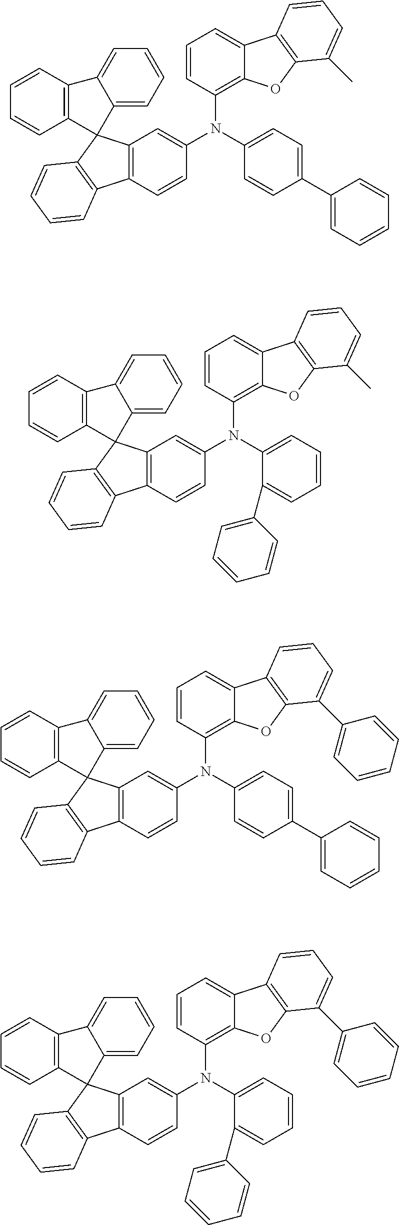 [SMILES: Cc1cccc2c1oc1c(N(c3ccc(-c4ccccc4)cc3)c3ccc4c(c3)C3(c5ccccc5-c5ccccc53)c3ccccc3-4)cccc12 ; Cc1cccc2c1oc1c(N(c3ccc4c(c3)C3(c5ccccc5-c5ccccc53)c3ccccc3-4)c3ccccc3-c3ccccc3)cccc12 ; c1ccc(-c2ccc(N(c3ccc4c(c3)C3(c5ccccc5-c5ccccc53)c3ccccc3-4)c3cccc4c3oc3c(-c5ccccc5)cccc34)cc2)cc1 ; c1ccc(-c2ccccc2N(c2ccc3c(c2)C2(c4ccccc4-c4ccccc42)c2ccccc2-3)c2cccc3c2oc2c(-c4ccccc4)cccc23)cc1]

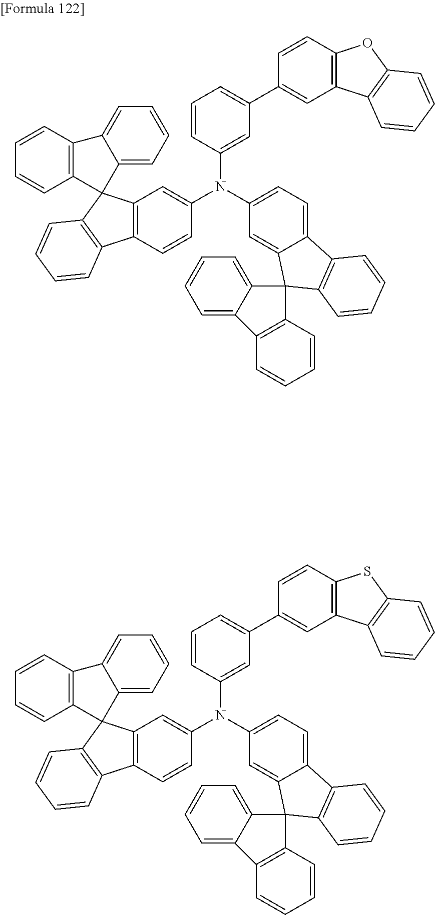 [SMILES: c1cc(-c2ccc3oc4ccccc4c3c2)cc(N(c2ccc3c(c2)C2(c4ccccc4-c4ccccc42)c2ccccc2-3)c2ccc3c(c2)C2(c4ccccc4-c4ccccc42)c2ccccc2-3)c1 ; c1cc(-c2ccc3sc4ccccc4c3c2)cc(N(c2ccc3c(c2)C2(c4ccccc4-c4ccccc42)c2ccccc2-3)c2ccc3c(c2)C2(c4ccccc4-c4ccccc42)c2ccccc2-3)c1]

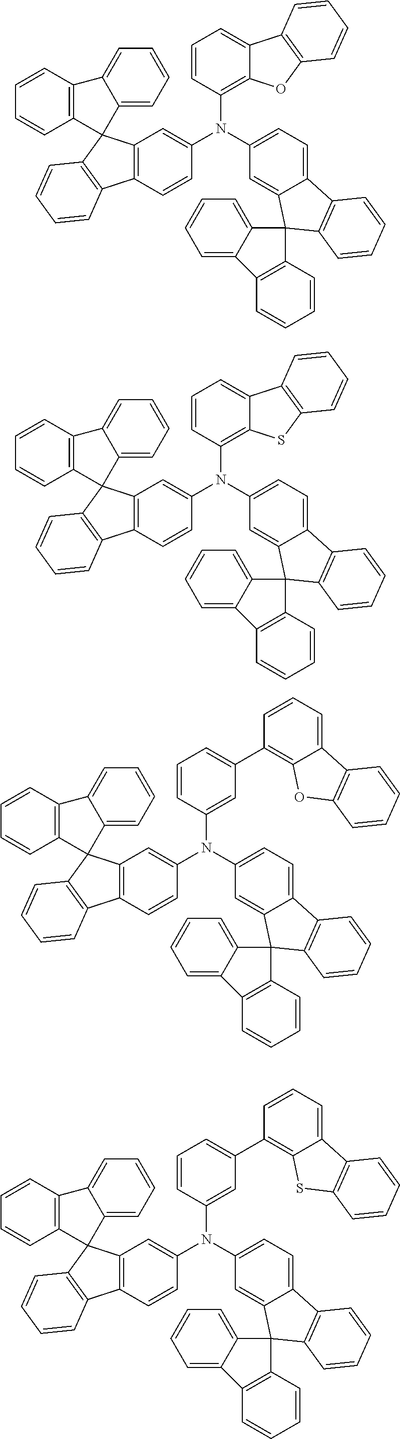

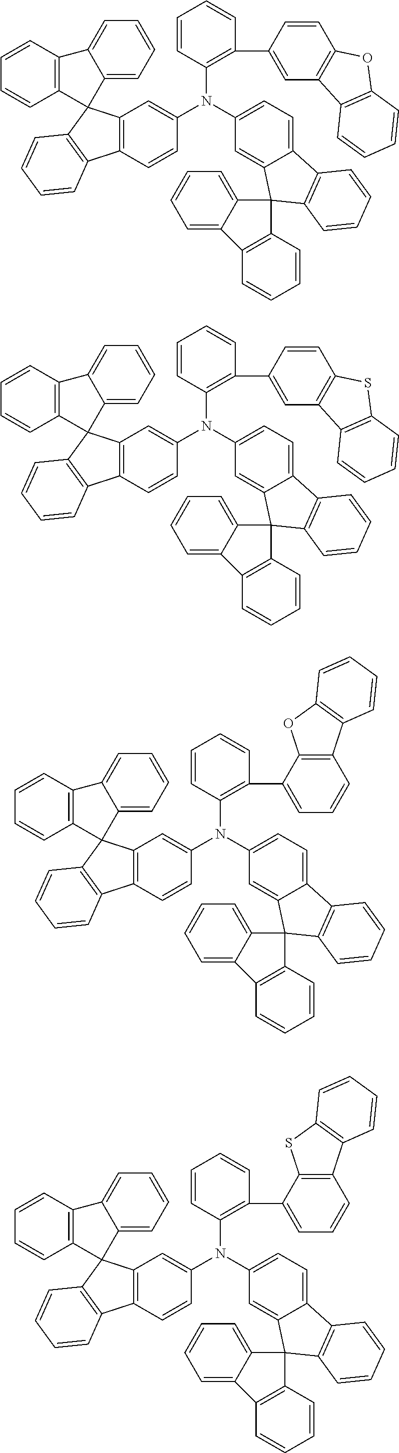 [SMILES: c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3oc4ccccc4c3c2)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2ccc3sc4ccccc4c3c2)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c2oc2ccccc23)c1 ; c1ccc(N(c2ccc3c(c2)C2(c4ccccc4-c4ccccc42)c2ccccc2-3)c2ccc3c(c2)C2(c4ccccc4-c4ccccc42)c2ccccc2-3)c(-c2cccc3c2sc2ccccc23)c1]

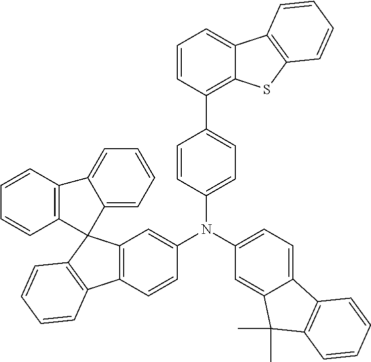 [SMILES: CC1(C)c2ccccc2-c2ccc(N(c3ccc(-c4cccc5c4sc4ccccc45)cc3)c3ccc4c(c3)C3(c5ccccc5-c5ccccc53)c3ccccc3-4)cc21]

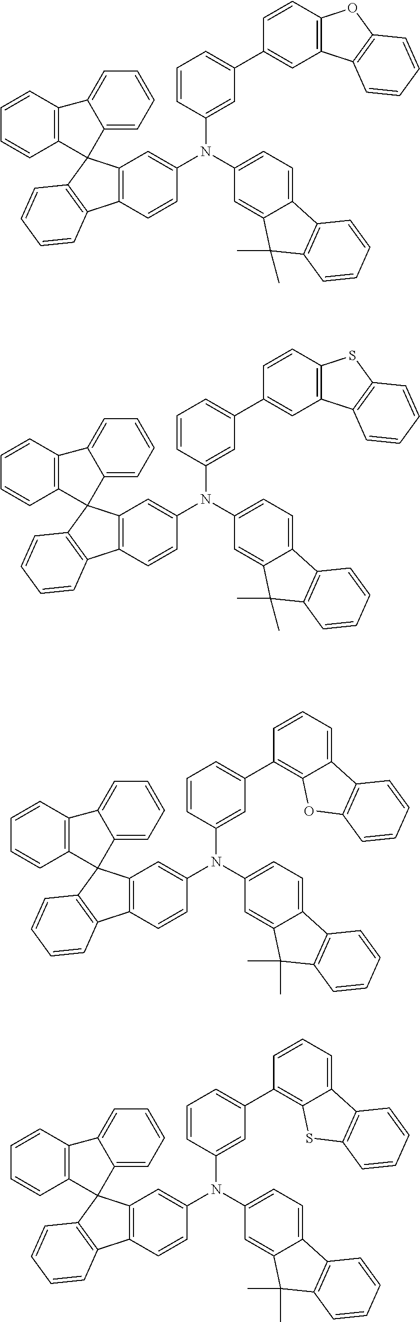 [SMILES: CC1(C)c2ccccc2-c2ccc(N(c3cccc(-c4ccc5oc6ccccc6c5c4)c3)c3ccc4c(c3)C3(c5ccccc5-c5ccccc53)c3ccccc3-4)cc21 ; CC1(C)c2ccccc2-c2ccc(N(c3cccc(-c4ccc5sc6ccccc6c5c4)c3)c3ccc4c(c3)C3(c5ccccc5-c5ccccc53)c3ccccc3-4)cc21 ; CC1(C)c2ccccc2-c2ccc(N(c3cccc(-c4cccc5c4oc4ccccc45)c3)c3ccc4c(c3)C3(c5ccccc5-c5ccccc53)c3ccccc3-4)cc21 ; CC1(C)c2ccccc2-c2ccc(N(c3cccc(-c4cccc5c4sc4ccccc45)c3)c3ccc4c(c3)C3(c5ccccc5-c5ccccc53)c3ccccc3-4)cc21]

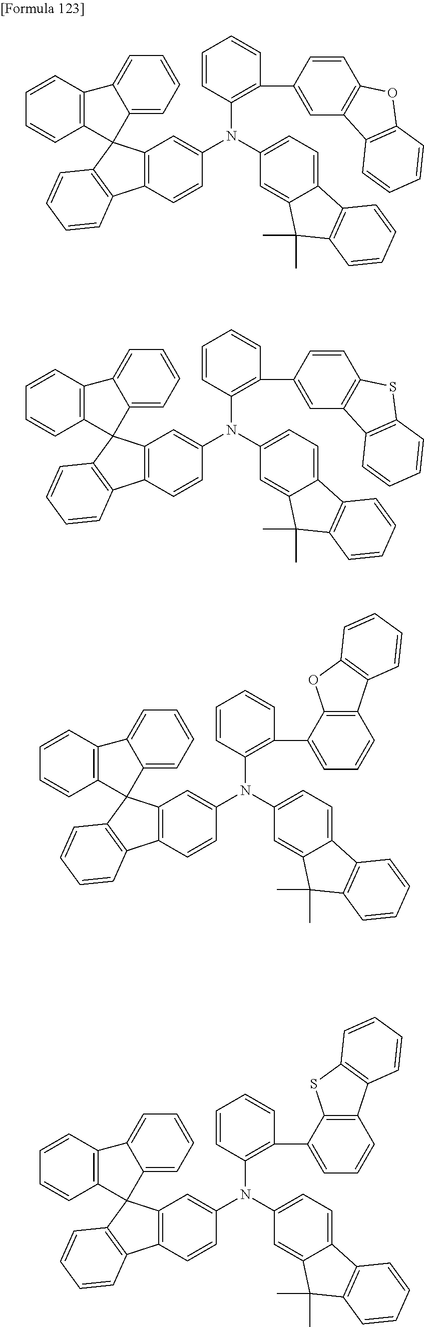 [SMILES: CC1(C)c2ccccc2-c2ccc(N(c3ccc4c(c3)C3(c5ccccc5-c5ccccc53)c3ccccc3-4)c3ccccc3-c3ccc4oc5ccccc5c4c3)cc21 ; CC1(C)c2ccccc2-c2ccc(N(c3ccc4c(c3)C3(c5ccccc5-c5ccccc53)c3ccccc3-4)c3ccccc3-c3ccc4sc5ccccc5c4c3)cc21 ; CC1(C)c2ccccc2-c2ccc(N(c3ccc4c(c3)C3(c5ccccc5-c5ccccc53)c3ccccc3-4)c3ccccc3-c3cccc4c3oc3ccccc34)cc21 ; CC1(C)c2ccccc2-c2ccc(N(c3ccc4c(c3)C3(c5ccccc5-c5ccccc53)c3ccccc3-4)c3ccccc3-c3cccc4c3sc3ccccc34)cc21]

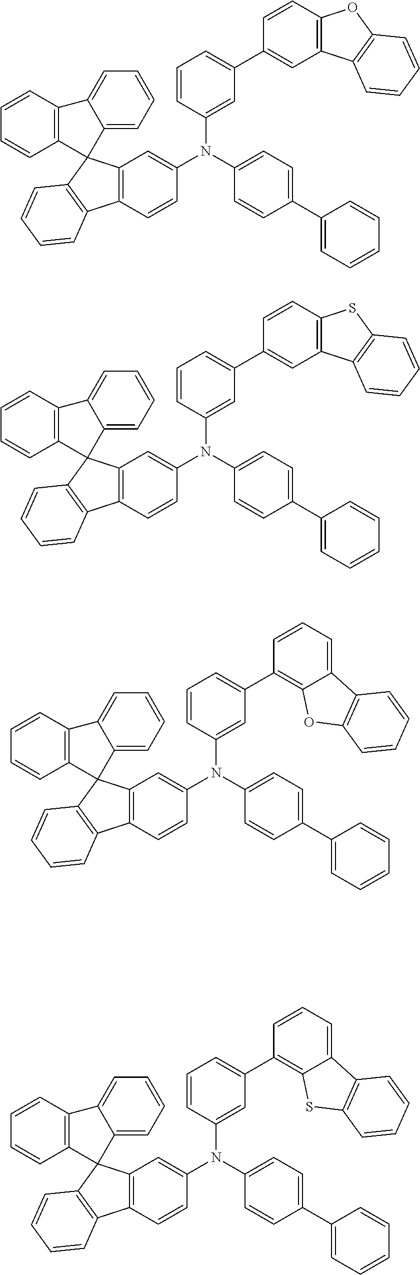

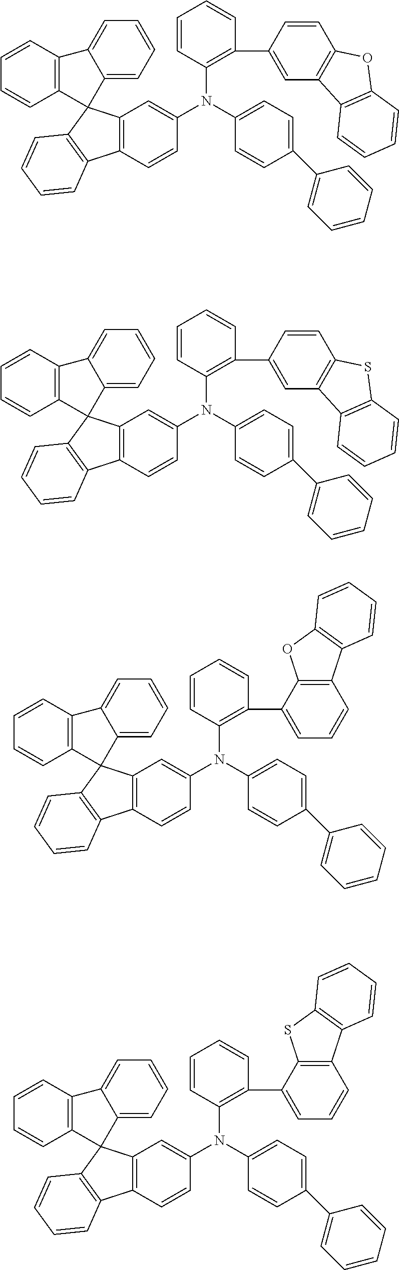

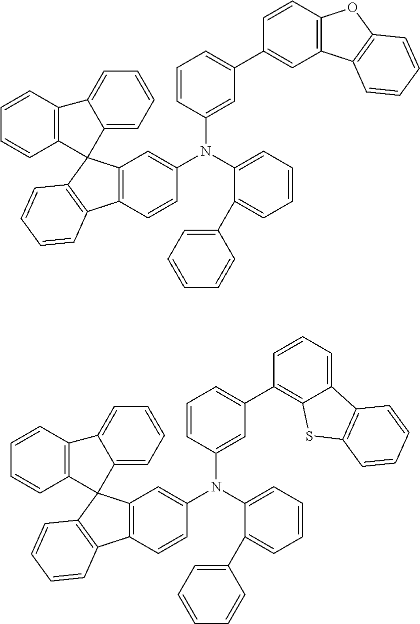 [SMILES: c1ccc(-c2ccccc2N(c2cccc(-c3ccc4oc5ccccc5c4c3)c2)c2ccc3c(c2)C2(c4ccccc4-c4ccccc42)c2ccccc2-3)cc1 ; c1ccc(-c2ccccc2N(c2cccc(-c3cccc4c3sc3ccccc34)c2)c2ccc3c(c2)C2(c4ccccc4-c4ccccc42)c2ccccc2-3)cc1]

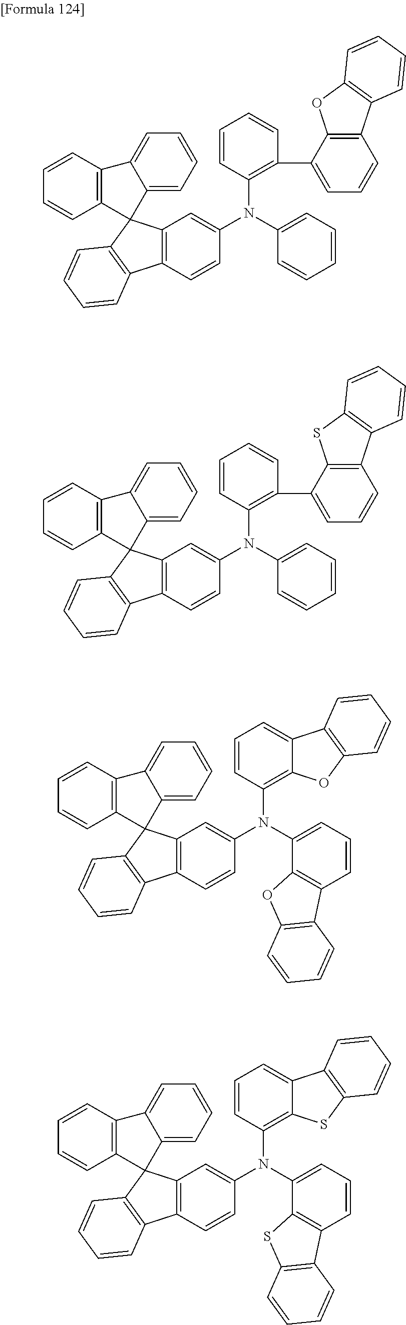 [SMILES: c1ccc(N(c2ccc3c(c2)C2(c4ccccc4-c4ccccc42)c2ccccc2-3)c2ccccc2-c2cccc3c2oc2ccccc23)cc1 ; c1ccc(N(c2ccc3c(c2)C2(c4ccccc4-c4ccccc42)c2ccccc2-3)c2ccccc2-c2cccc3c2sc2ccccc23)cc1 ; c1ccc2c(c1)-c1ccccc1C21c2ccccc2-c2ccc(N(c3cccc4c3oc3ccccc34)c3cccc4c3oc3ccccc34)cc21 ; c1ccc2c(c1)-c1ccccc1C21c2ccccc2-c2ccc(N(c3cccc4c3sc3ccccc34)c3cccc4c3sc3ccccc34)cc21]

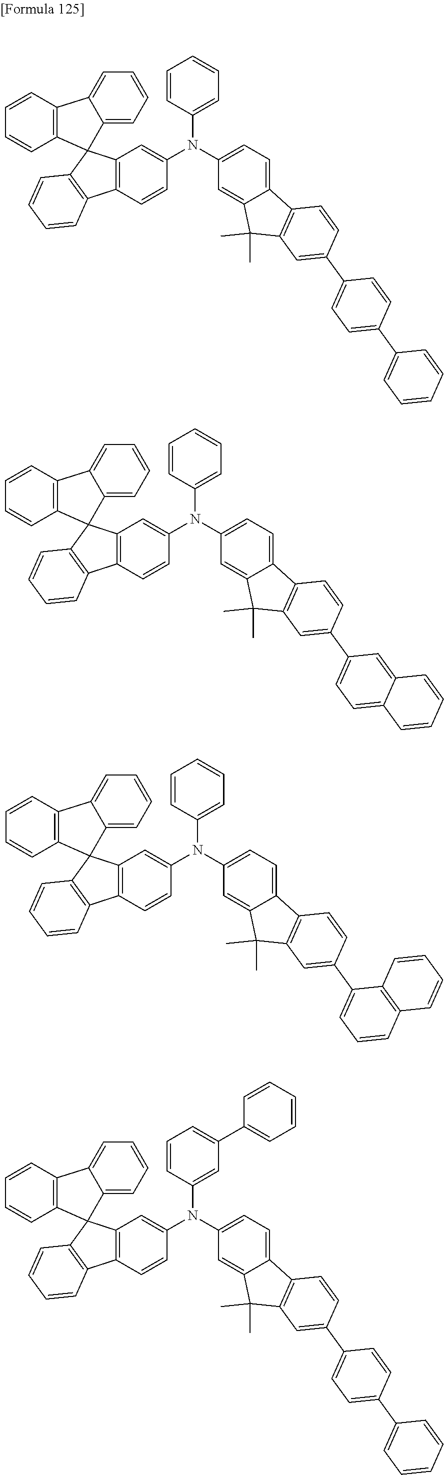 [SMILES: CC1(C)c2cc(-c3ccc(-c4ccccc4)cc3)ccc2-c2ccc(N(c3ccccc3)c3ccc4c(c3)C3(c5ccccc5-c5ccccc53)c3ccccc3-4)cc21 ; CC1(C)c2cc(-c3ccc4ccccc4c3)ccc2-c2ccc(N(c3ccccc3)c3ccc4c(c3)C3(c5ccccc5-c5ccccc53)c3ccccc3-4)cc21 ; CC1(C)c2cc(-c3cccc4ccccc34)ccc2-c2ccc(N(c3ccccc3)c3ccc4c(c3)C3(c5ccccc5-c5ccccc53)c3ccccc3-4)cc21 ; CC1(C)c2cc(-c3ccc(-c4ccccc4)cc3)ccc2-c2ccc(N(c3cccc(-c4ccccc4)c3)c3ccc4c(c3)C3(c5ccccc5-c5ccccc53)c3ccccc3-4)cc21]

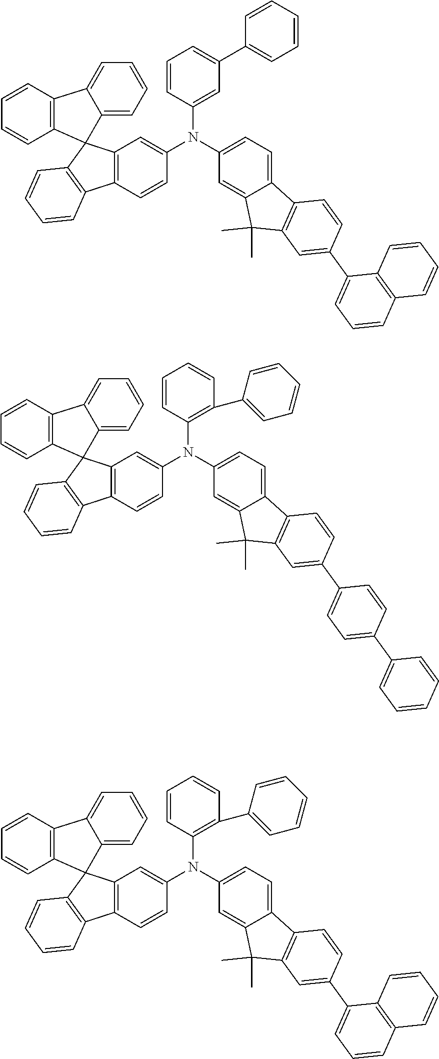 [SMILES: CC1(C)c2cc(-c3cccc4ccccc34)ccc2-c2ccc(N(c3cccc(-c4ccccc4)c3)c3ccc4c(c3)C3(c5ccccc5-c5ccccc53)c3ccccc3-4)cc21 ; CC1(C)c2cc(-c3ccc(-c4ccccc4)cc3)ccc2-c2ccc(N(c3ccc4c(c3)C3(c5ccccc5-c5ccccc53)c3ccccc3-4)c3ccccc3-c3ccccc3)cc21 ; CC1(C)c2cc(-c3cccc4ccccc34)ccc2-c2ccc(N(c3ccc4c(c3)C3(c5ccccc5-c5ccccc53)c3ccccc3-4)c3ccccc3-c3ccccc3)cc21]

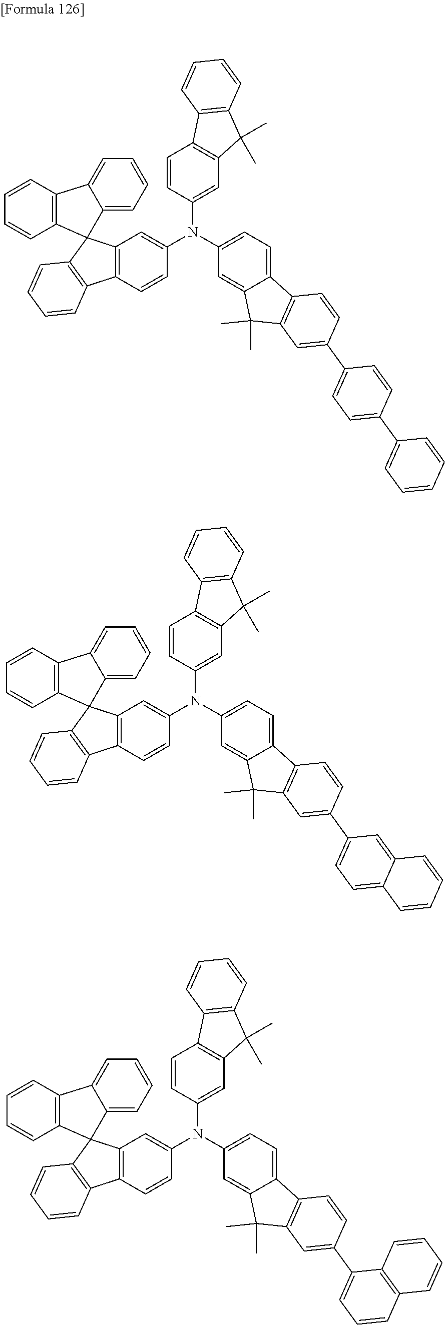 [SMILES: CC1(C)c2ccccc2-c2ccc(N(c3ccc4c(c3)C(C)(C)c3cc(-c5ccc(-c6ccccc6)cc5)ccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(C)c3cc(-c5ccc6ccccc6c5)ccc3-4)c3ccc4c(c3)C3(c5ccccc5-c5ccccc53)c3ccccc3-4)cc21 ; CC1(C)c2ccccc2-c2ccc(N(c3ccc4c(c3)C(C)(C)c3cc(-c5cccc6ccccc56)ccc3-4)c3ccc4c(c3)C3(c5ccccc5-c5ccccc53)c3ccccc3-4)cc21]

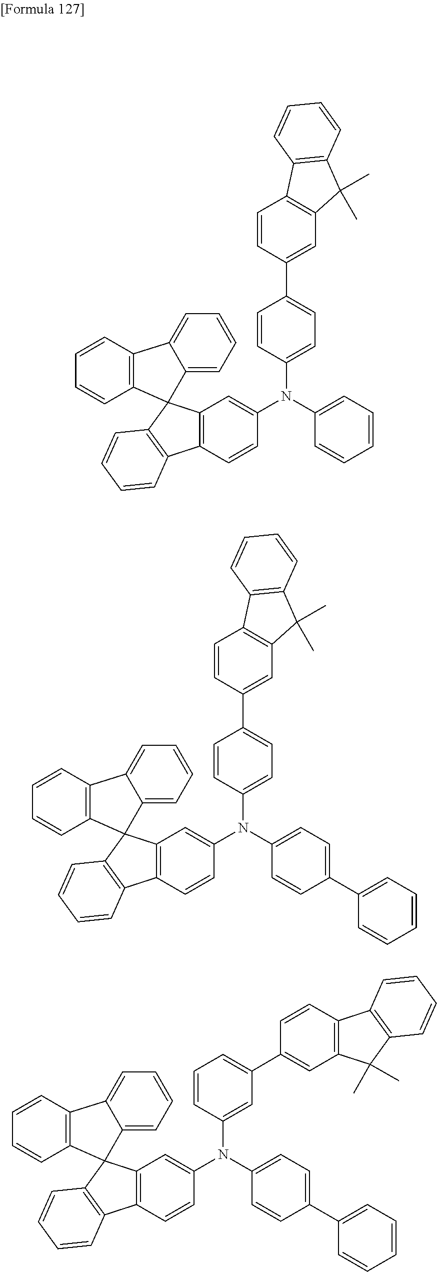

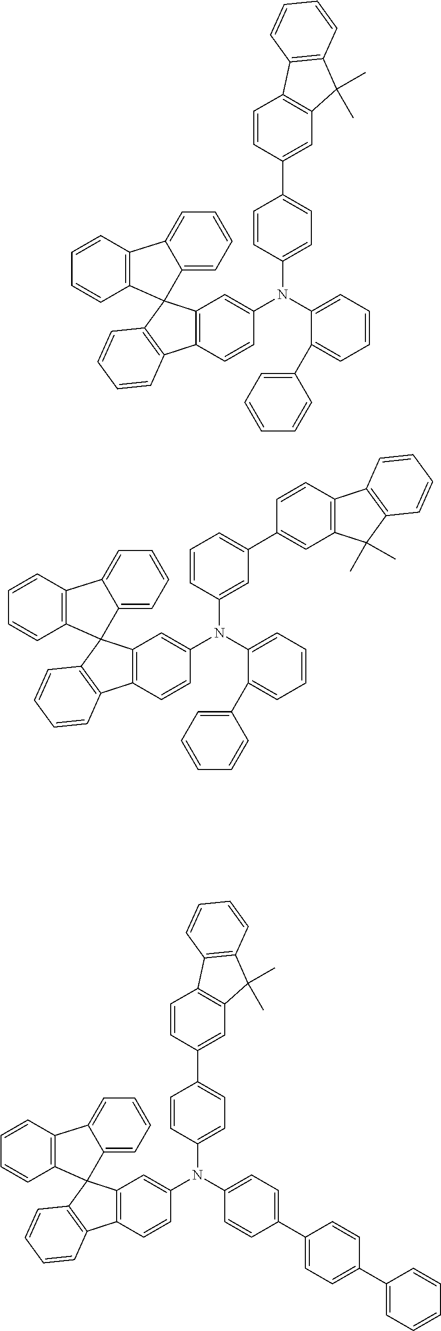 [SMILES: CC1(C)c2ccccc2-c2ccc(-c3ccc(N(c4ccc5c(c4)C4(c6ccccc6-c6ccccc64)c4ccccc4-5)c4ccccc4-c4ccccc4)cc3)cc21 ; CC1(C)c2ccccc2-c2ccc(-c3cccc(N(c4ccc5c(c4)C4(c6ccccc6-c6ccccc64)c4ccccc4-5)c4ccccc4-c4ccccc4)c3)cc21 ; CC1(C)c2ccccc2-c2ccc(-c3ccc(N(c4ccc(-c5ccc(-c6ccccc6)cc5)cc4)c4ccc5c(c4)C4(c6ccccc6-c6ccccc64)c4ccccc4-5)cc3)cc21]

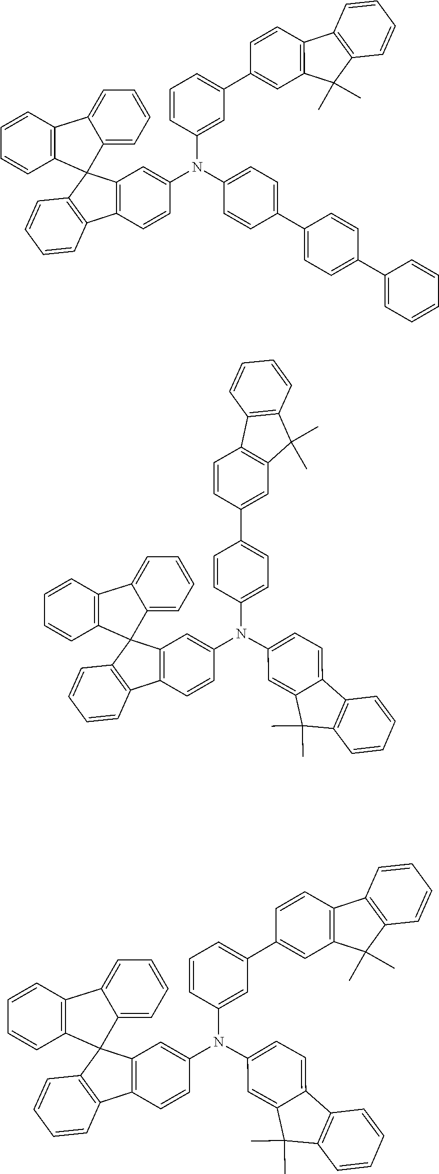 [SMILES: CC1(C)c2ccccc2-c2ccc(-c3cccc(N(c4ccc(-c5ccc(-c6ccccc6)cc5)cc4)c4ccc5c(c4)C4(c6ccccc6-c6ccccc64)c4ccccc4-5)c3)cc21 ; CC1(C)c2ccccc2-c2ccc(-c3ccc(N(c4ccc5c(c4)C(C)(C)c4ccccc4-5)c4ccc5c(c4)C4(c6ccccc6-c6ccccc64)c4ccccc4-5)cc3)cc21 ; CC1(C)c2ccccc2-c2ccc(-c3cccc(N(c4ccc5c(c4)C(C)(C)c4ccccc4-5)c4ccc5c(c4)C4(c6ccccc6-c6ccccc64)c4ccccc4-5)c3)cc21]

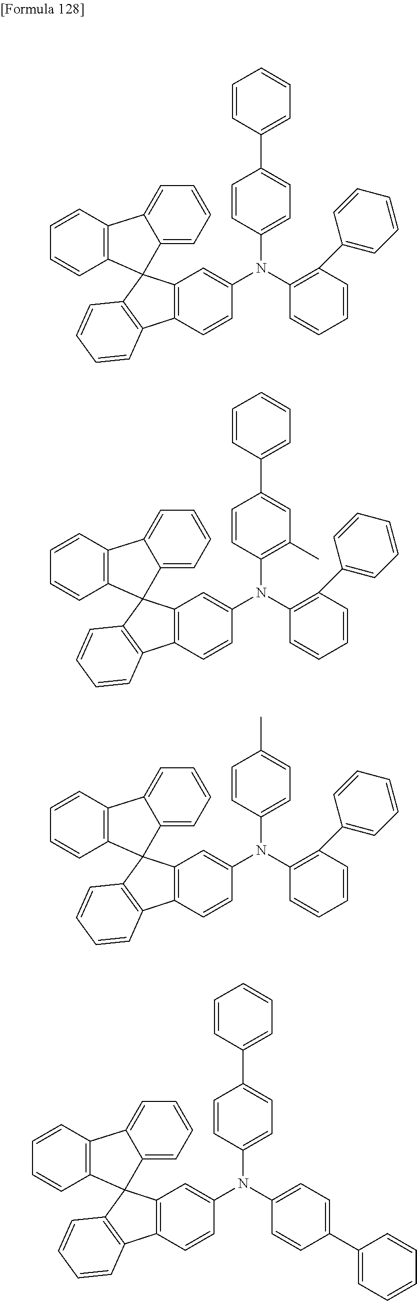 [SMILES: c1ccc(-c2ccc(N(c3ccc4c(c3)C3(c5ccccc5-c5ccccc53)c3ccccc3-4)c3ccccc3-c3ccccc3)cc2)cc1 ; Cc1cc(-c2ccccc2)ccc1N(c1ccc2c(c1)C1(c3ccccc3-c3ccccc31)c1ccccc1-2)c1ccccc1-c1ccccc1 ; Cc1ccc(N(c2ccc3c(c2)C2(c4ccccc4-c4ccccc42)c2ccccc2-3)c2ccccc2-c2ccccc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1]

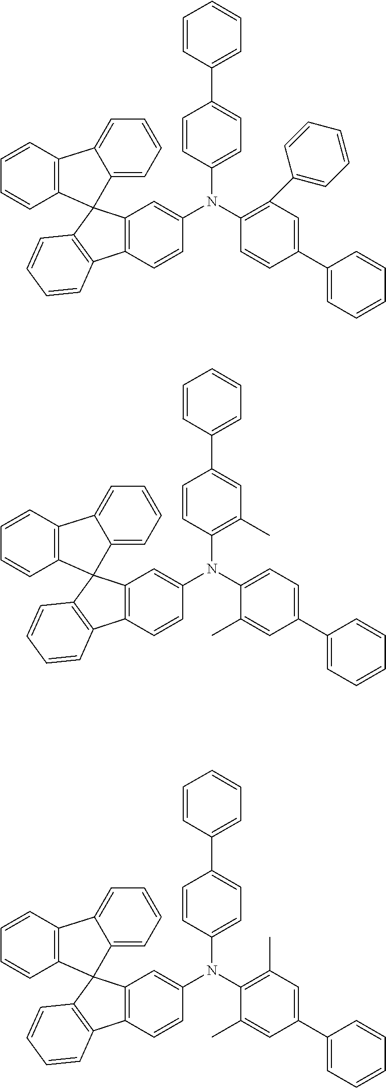 [SMILES: c1ccc(-c2ccc(N(c3ccc4c(c3)C3(c5ccccc5-c5ccccc53)c3ccccc3-4)c3ccc(-c4ccccc4)cc3-c3ccccc3)cc2)cc1 ; Cc1cc(-c2ccccc2)ccc1N(c1ccc2c(c1)C1(c3ccccc3-c3ccccc31)c1ccccc1-2)c1ccc(-c2ccccc2)cc1C ; Cc1cc(-c2ccccc2)cc(C)c1N(c1ccc(-c2ccccc2)cc1)c1ccc2c(c1)C1(c3ccccc3-c3ccccc31)c1ccccc1-2]

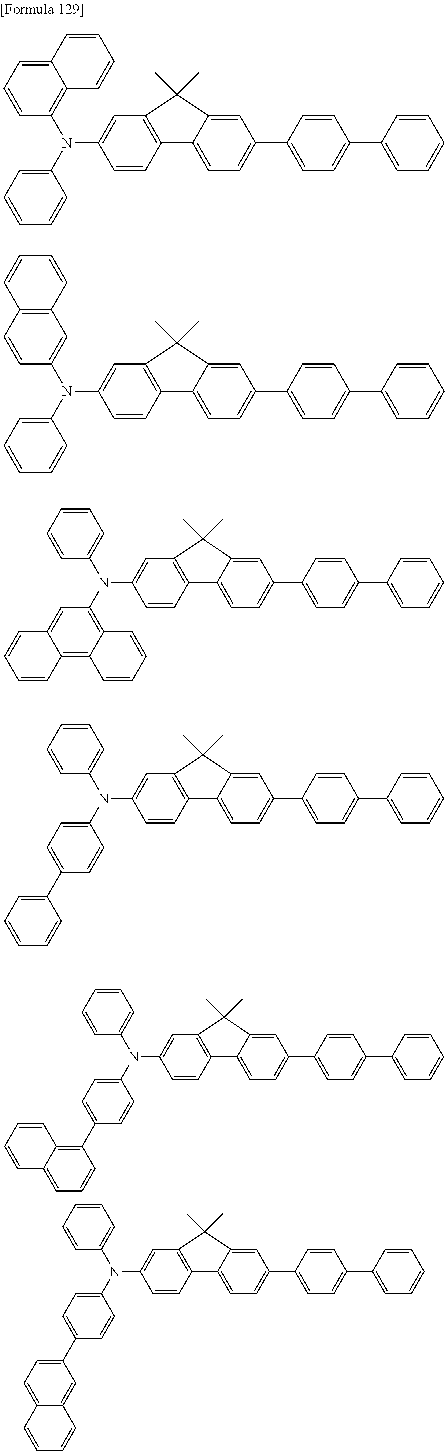

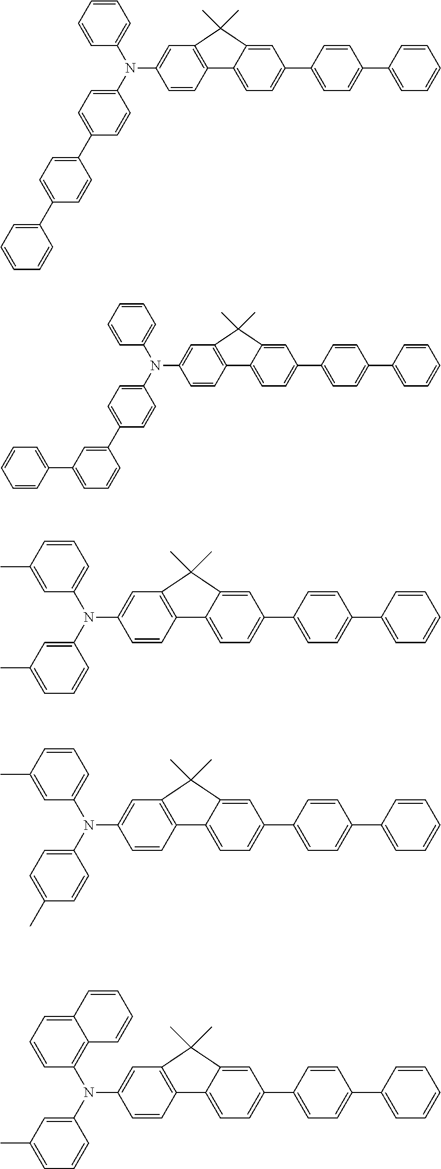 [SMILES: CC1(C)c2cc(-c3ccc(-c4ccccc4)cc3)ccc2-c2ccc(N(c3ccccc3)c3ccc(-c4ccc(-c5ccccc5)cc4)cc3)cc21 ; CC1(C)c2cc(-c3ccc(-c4ccccc4)cc3)ccc2-c2ccc(N(c3ccccc3)c3ccc(-c4cccc(-c5ccccc5)c4)cc3)cc21 ; Cc1cccc(N(c2cccc(C)c2)c2ccc3c(c2)C(C)(C)c2cc(-c4ccc(-c5ccccc5)cc4)ccc2-3)c1 ; Cc1ccc(N(c2cccc(C)c2)c2ccc3c(c2)C(C)(C)c2cc(-c4ccc(-c5ccccc5)cc4)ccc2-3)cc1 ; Cc1cccc(N(c2ccc3c(c2)C(C)(C)c2cc(-c4ccc(-c5ccccc5)cc4)ccc2-3)c2cccc3ccccc23)c1]

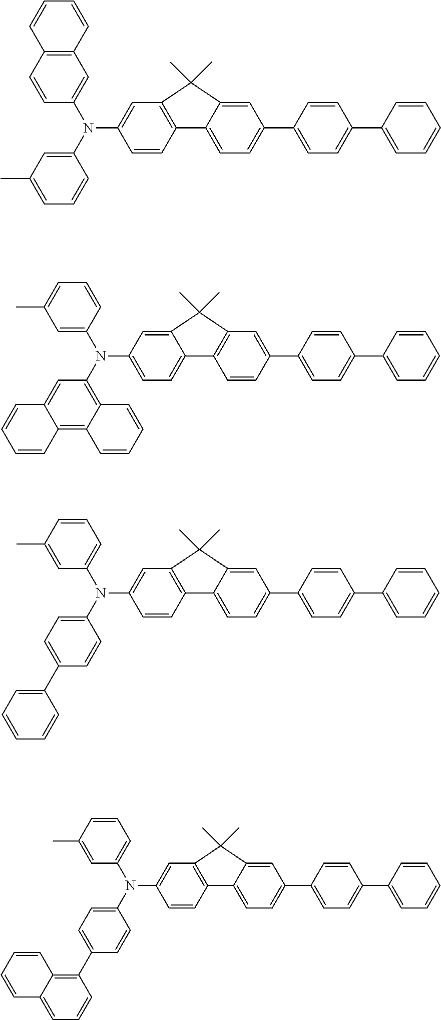 [SMILES: Cc1cccc(N(c2ccc3c(c2)C(C)(C)c2cc(-c4ccc(-c5ccccc5)cc4)ccc2-3)c2ccc3ccccc3c2)c1 ; Cc1cccc(N(c2ccc3c(c2)C(C)(C)c2cc(-c4ccc(-c5ccccc5)cc4)ccc2-3)c2cc3ccccc3c3ccccc23)c1 ; Cc1cccc(N(c2ccc(-c3ccccc3)cc2)c2ccc3c(c2)C(C)(C)c2cc(-c4ccc(-c5ccccc5)cc4)ccc2-3)c1 ; Cc1cccc(N(c2ccc(-c3cccc4ccccc34)cc2)c2ccc3c(c2)C(C)(C)c2cc(-c4ccc(-c5ccccc5)cc4)ccc2-3)c1]

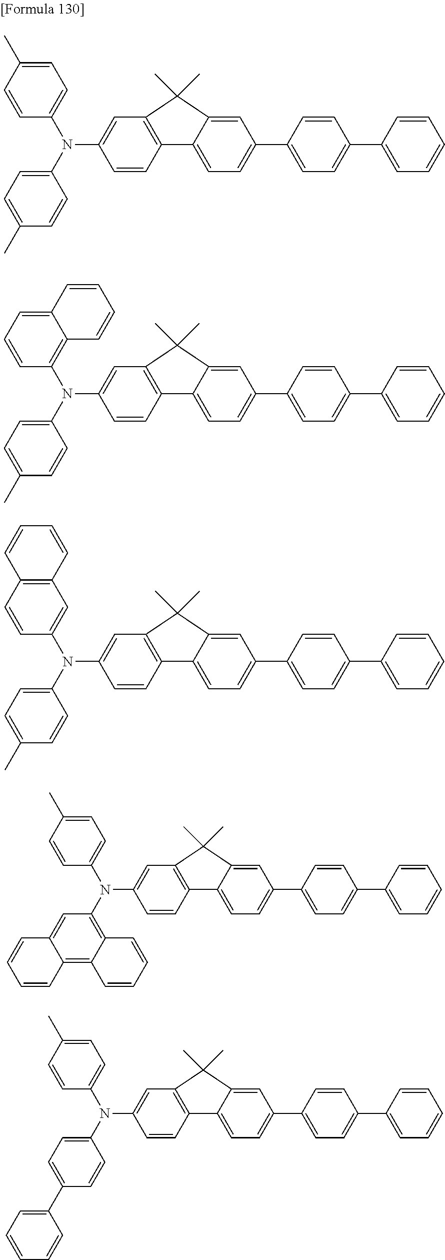

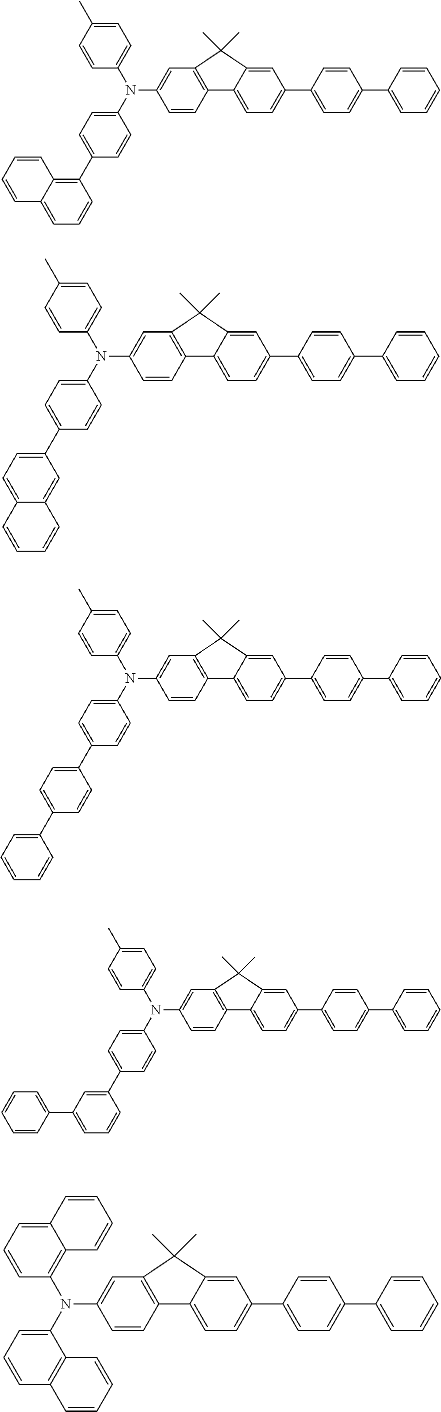

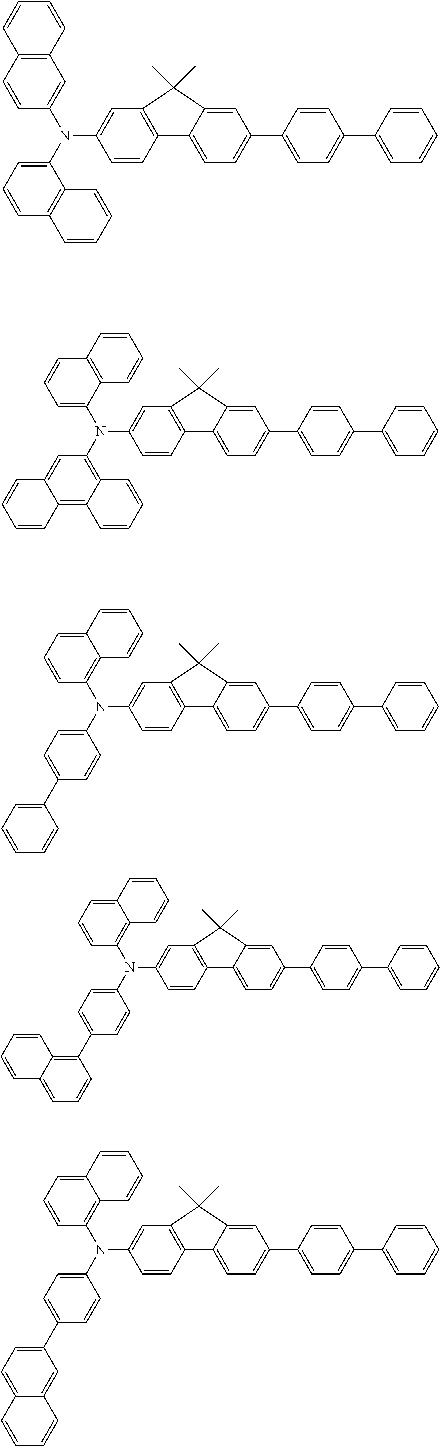 [SMILES: CC1(C)c2cc(-c3ccc(-c4ccccc4)cc3)ccc2-c2ccc(N(c3ccc4ccccc4c3)c3cccc4ccccc34)cc21 ; CC1(C)c2cc(-c3ccc(-c4ccccc4)cc3)ccc2-c2ccc(N(c3cccc4ccccc34)c3cc4ccccc4c4ccccc34)cc21 ; CC1(C)c2cc(-c3ccc(-c4ccccc4)cc3)ccc2-c2ccc(N(c3ccc(-c4ccccc4)cc3)c3cccc4ccccc34)cc21 ; CC1(C)c2cc(-c3ccc(-c4ccccc4)cc3)ccc2-c2ccc(N(c3ccc(-c4cccc5ccccc45)cc3)c3cccc4ccccc34)cc21 ; CC1(C)c2cc(-c3ccc(-c4ccccc4)cc3)ccc2-c2ccc(N(c3ccc(-c4ccc5ccccc5c4)cc3)c3cccc4ccccc34)cc21]

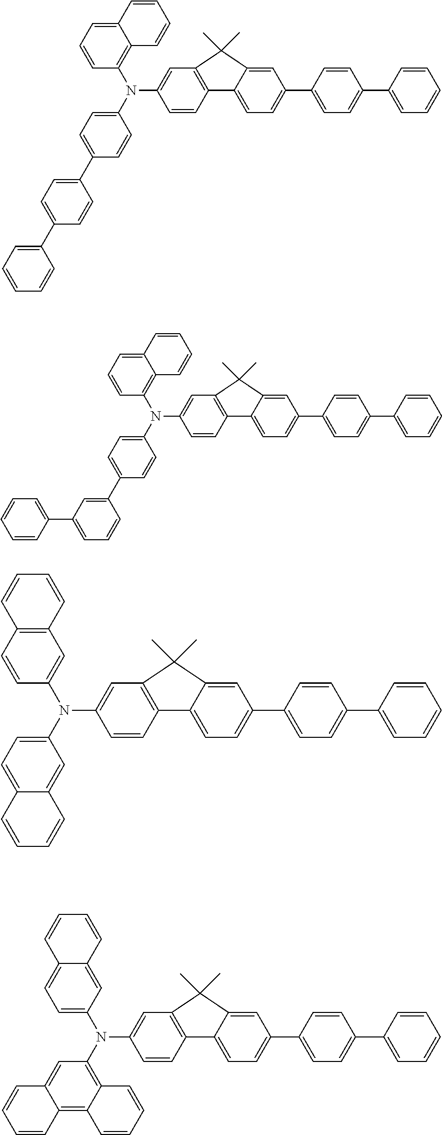

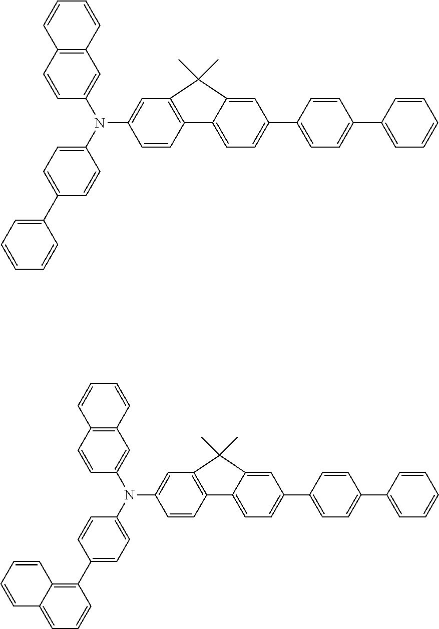 [SMILES: CC1(C)c2cc(-c3ccc(-c4ccccc4)cc3)ccc2-c2ccc(N(c3ccc(-c4ccccc4)cc3)c3ccc4ccccc4c3)cc21 ; CC1(C)c2cc(-c3ccc(-c4ccccc4)cc3)ccc2-c2ccc(N(c3ccc(-c4cccc5ccccc45)cc3)c3ccc4ccccc4c3)cc21]

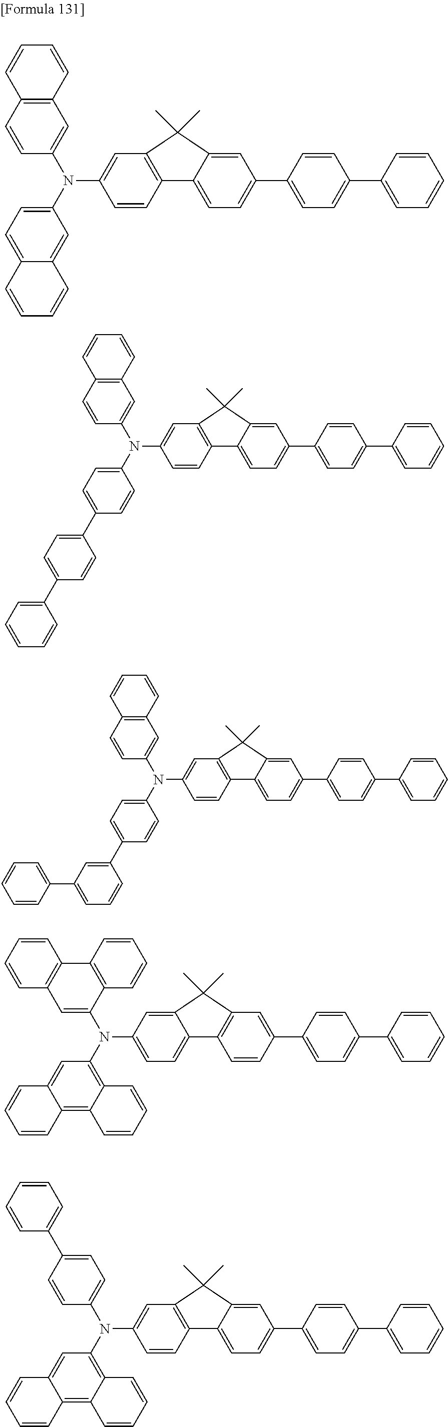

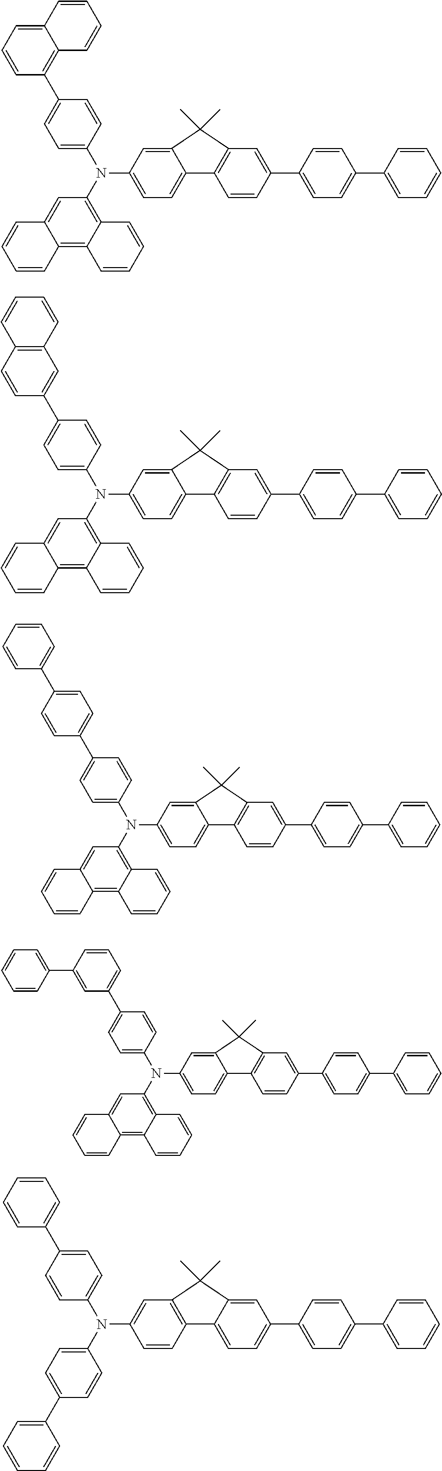

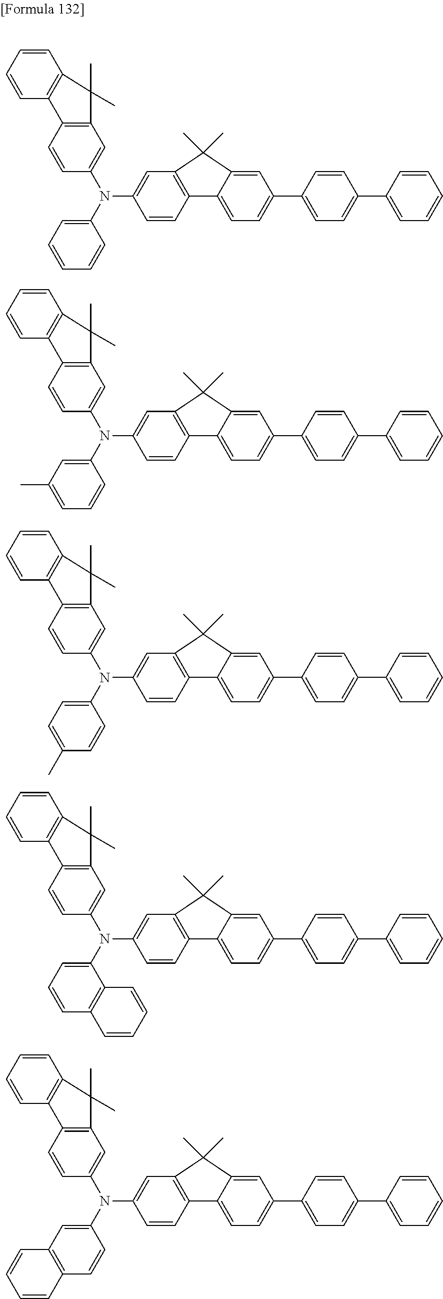

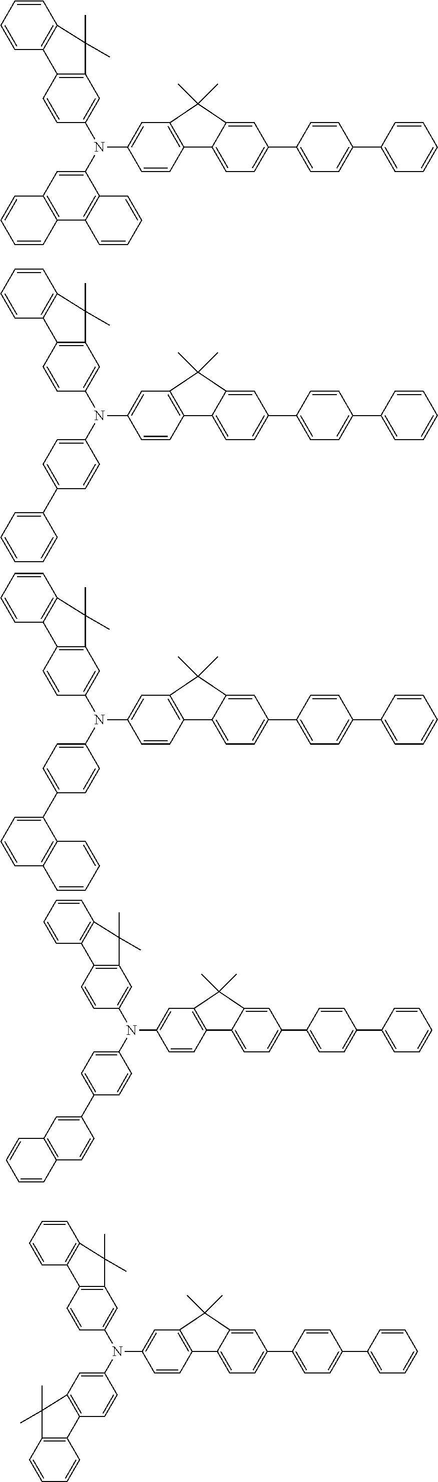

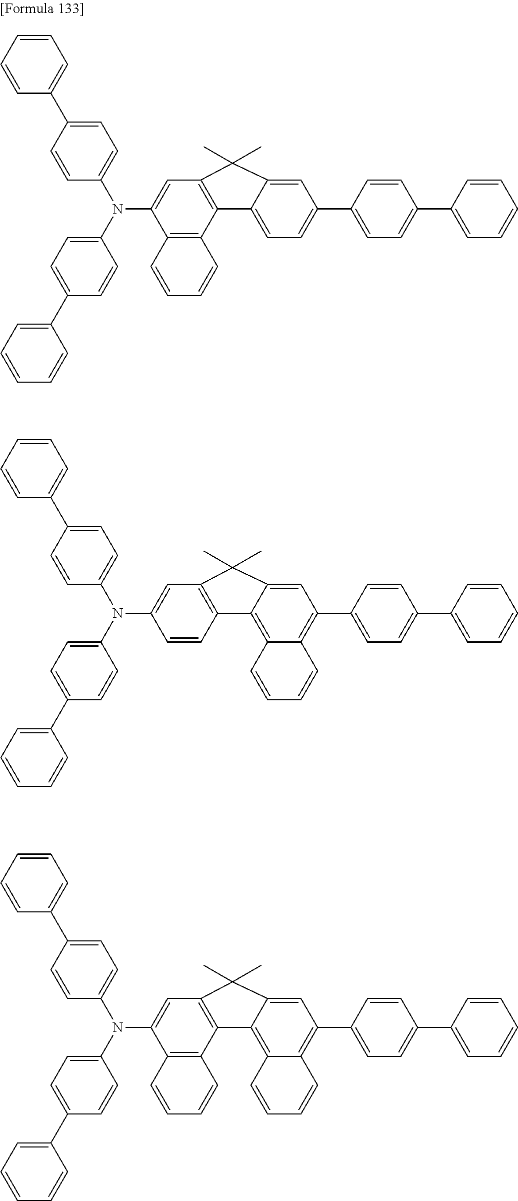 [SMILES: CC1(C)c2cc(-c3ccc(-c4ccccc4)cc3)ccc2-c2c1cc(N(c1ccc(-c3ccccc3)cc1)c1ccc(-c3ccccc3)cc1)c1ccccc21 ; CC1(C)c2cc(N(c3ccc(-c4ccccc4)cc3)c3ccc(-c4ccccc4)cc3)ccc2-c2c1cc(-c1ccc(-c3ccccc3)cc1)c1ccccc21 ; CC1(C)c2cc(-c3ccc(-c4ccccc4)cc3)c3ccccc3c2-c2c1cc(N(c1ccc(-c3ccccc3)cc1)c1ccc(-c3ccccc3)cc1)c1ccccc21]

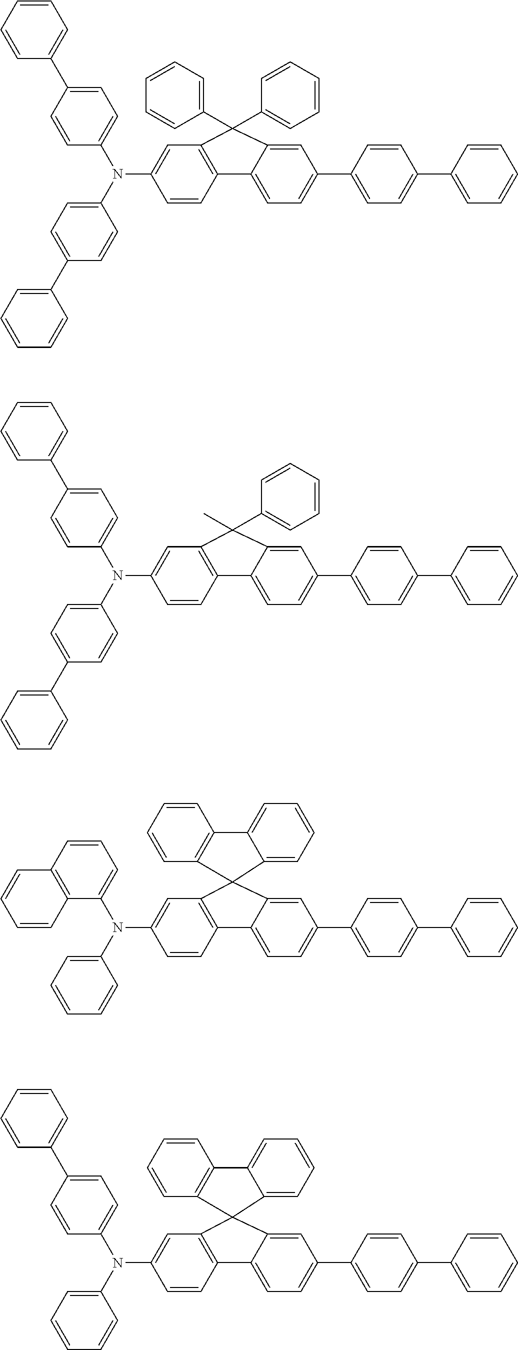 [SMILES: c1ccc(-c2ccc(-c3ccc4c(c3)C(c3ccccc3)(c3ccccc3)c3cc(N(c5ccc(-c6ccccc6)cc5)c5ccc(-c6ccccc6)cc5)ccc3-4)cc2)cc1 ; CC1(c2ccccc2)c2cc(-c3ccc(-c4ccccc4)cc3)ccc2-c2ccc(N(c3ccc(-c4ccccc4)cc3)c3ccc(-c4ccccc4)cc3)cc21 ; c1ccc(-c2ccc(-c3ccc4c(c3)C3(c5ccccc5-c5ccccc53)c3cc(N(c5ccccc5)c5cccc6ccccc56)ccc3-4)cc2)cc1 ; c1ccc(-c2ccc(-c3ccc4c(c3)C3(c5ccccc5-c5ccccc53)c3cc(N(c5ccccc5)c5ccc(-c6ccccc6)cc5)ccc3-4)cc2)cc1]

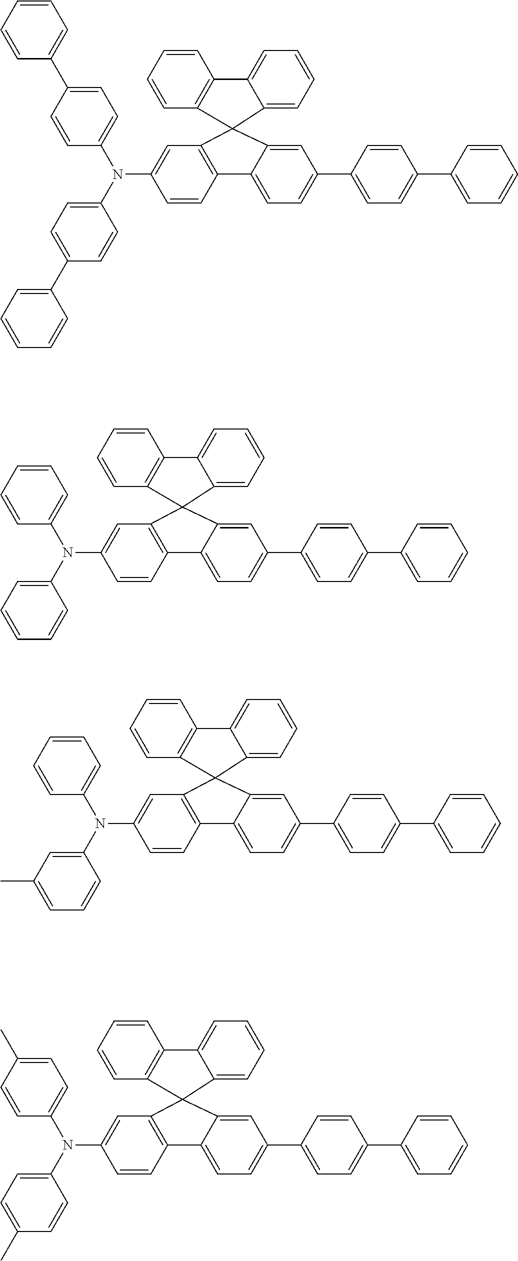 [SMILES: c1ccc(-c2ccc(-c3ccc4c(c3)C3(c5ccccc5-c5ccccc53)c3cc(N(c5ccc(-c6ccccc6)cc5)c5ccc(-c6ccccc6)cc5)ccc3-4)cc2)cc1 ; c1ccc(-c2ccc(-c3ccc4c(c3)C3(c5ccccc5-c5ccccc53)c3cc(N(c5ccccc5)c5ccccc5)ccc3-4)cc2)cc1 ; Cc1cccc(N(c2ccccc2)c2ccc3c(c2)C2(c4ccccc4-c4ccccc42)c2cc(-c4ccc(-c5ccccc5)cc4)ccc2-3)c1 ; Cc1ccc(N(c2ccc(C)cc2)c2ccc3c(c2)C2(c4ccccc4-c4ccccc42)c2cc(-c4ccc(-c5ccccc5)cc4)ccc2-3)cc1]

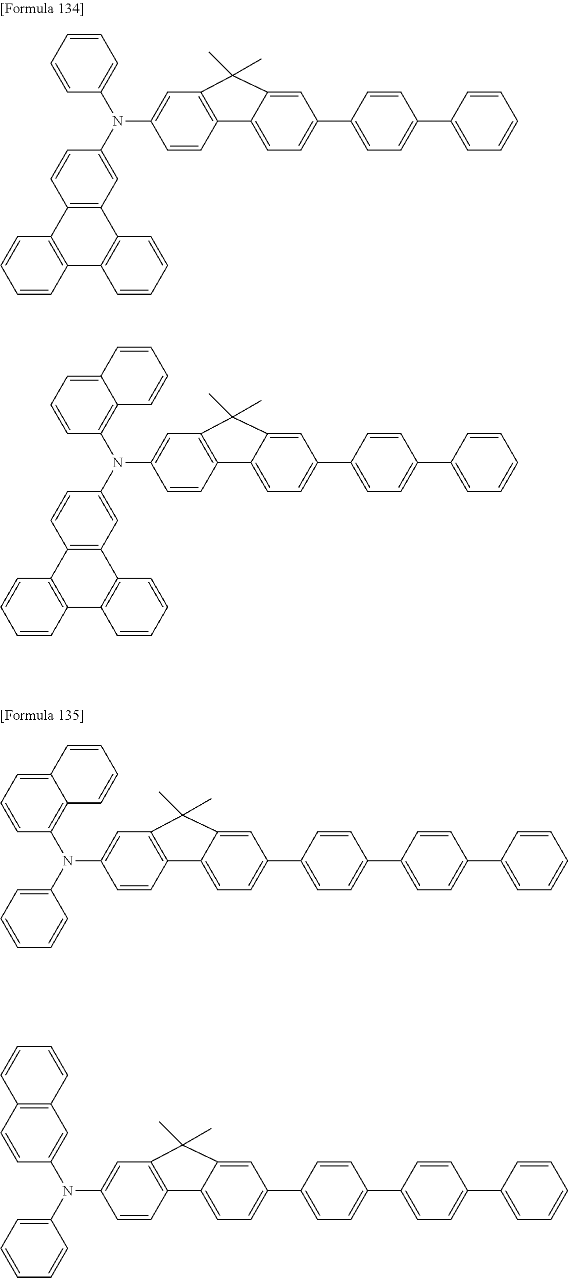 [SMILES: CC1(C)c2cc(-c3ccc(-c4ccccc4)cc3)ccc2-c2ccc(N(c3ccccc3)c3ccc4c5ccccc5c5ccccc5c4c3)cc21 ; CC1(C)c2cc(-c3ccc(-c4ccccc4)cc3)ccc2-c2ccc(N(c3ccc4c5ccccc5c5ccccc5c4c3)c3cccc4ccccc34)cc21 ; CC1(C)c2cc(-c3ccc(-c4ccc(-c5ccccc5)cc4)cc3)ccc2-c2ccc(N(c3ccccc3)c3cccc4ccccc34)cc21 ; CC1(C)c2cc(-c3ccc(-c4ccc(-c5ccccc5)cc4)cc3)ccc2-c2ccc(N(c3ccccc3)c3ccc4ccccc4c3)cc21]

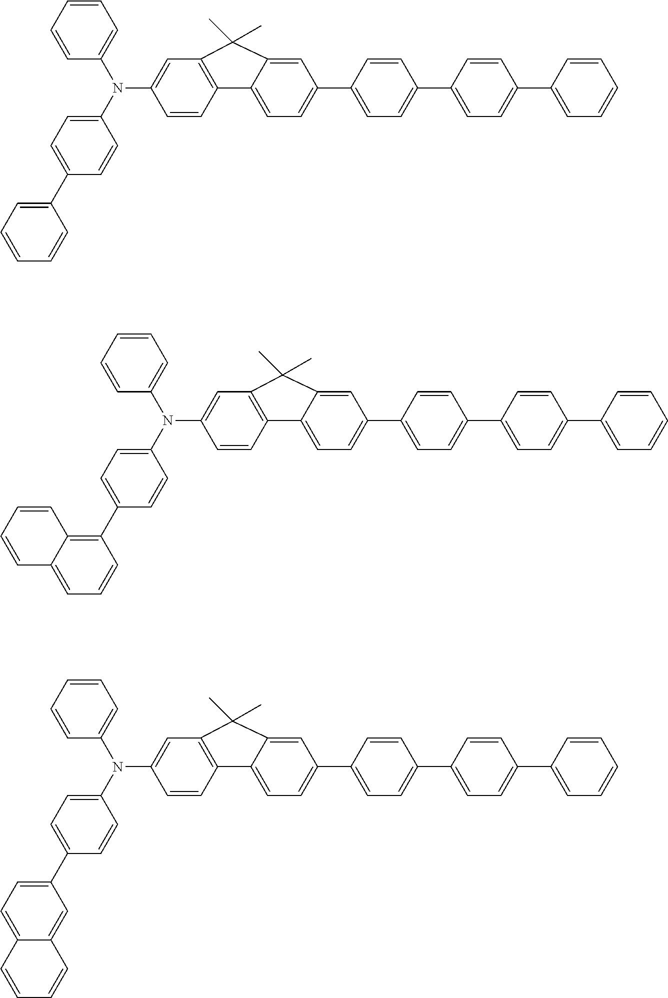 [SMILES: CC1(C)c2cc(-c3ccc(-c4ccc(-c5ccccc5)cc4)cc3)ccc2-c2ccc(N(c3ccccc3)c3ccc(-c4ccccc4)cc3)cc21 ; CC1(C)c2cc(-c3ccc(-c4ccc(-c5ccccc5)cc4)cc3)ccc2-c2ccc(N(c3ccccc3)c3ccc(-c4cccc5ccccc45)cc3)cc21 ; CC1(C)c2cc(-c3ccc(-c4ccc(-c5ccccc5)cc4)cc3)ccc2-c2ccc(N(c3ccccc3)c3ccc(-c4ccc5ccccc5c4)cc3)cc21]

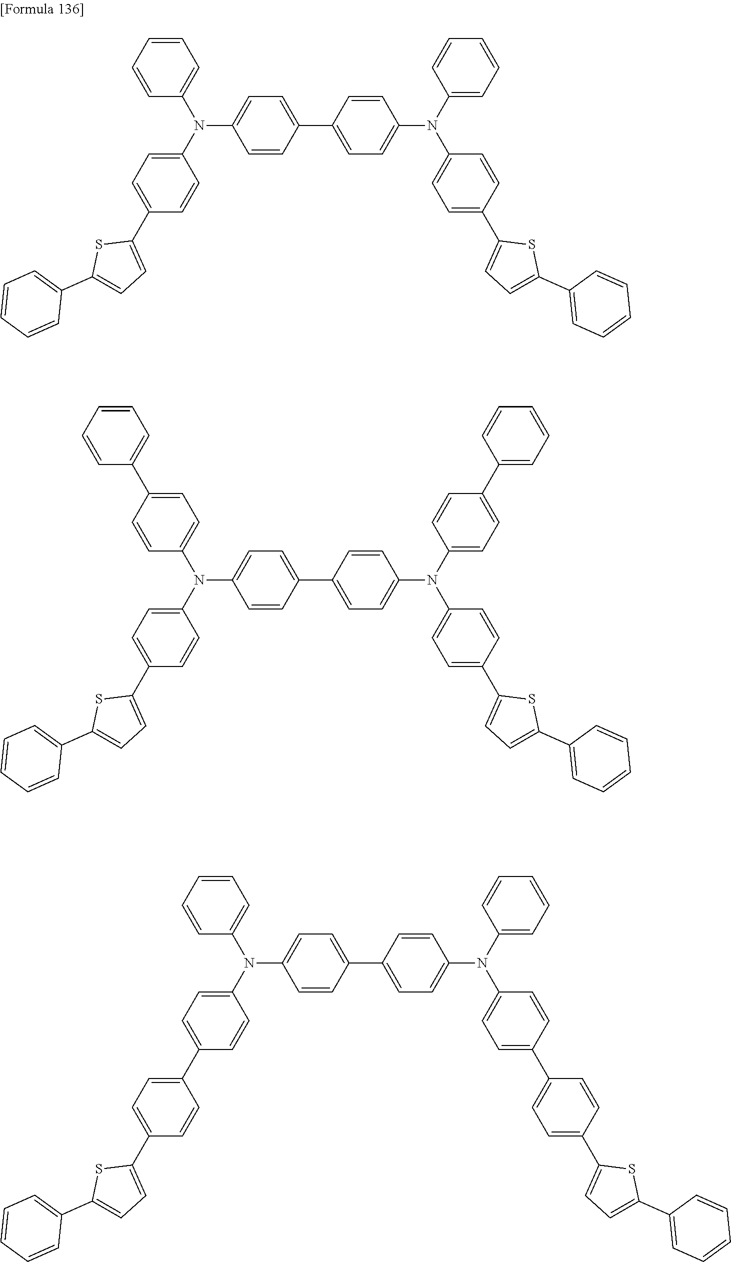 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; c1ccc(-c2ccc(N(c3ccc(-c4ccc(N(c5ccc(-c6ccccc6)cc5)c5ccc(-c6ccc(-c7ccccc7)s6)cc5)cc4)cc3)c3ccc(-c4ccc(-c5ccccc5)s4)cc3)cc2)cc1 ; c1ccc(-c2ccc(-c3ccc(-c4ccc(N(c5ccccc5)c5ccc(-c6ccc(N(c7ccccc7)c7ccc(-c8ccc(-c9ccc(-c%10ccccc%10)s9)cc8)cc7)cc6)cc5)cc4)cc3)s2)cc1]

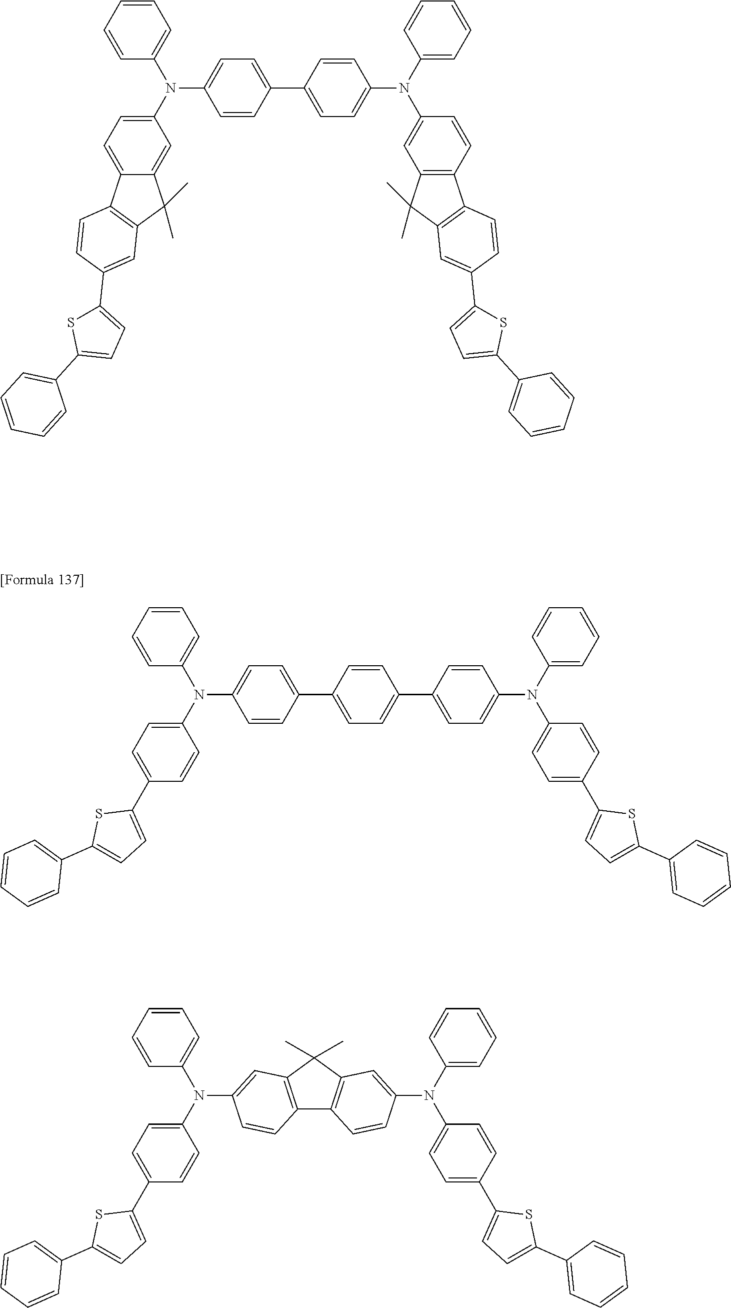 [SMILES: CC1(C)c2cc(-c3ccc(-c4ccccc4)s3)ccc2-c2ccc(N(c3ccccc3)c3ccc(-c4ccc(N(c5ccccc5)c5ccc6c(c5)C(C)(C)c5cc(-c7ccc(-c8ccccc8)s7)ccc5-6)cc4)cc3)cc21 ; c1ccc(-c2ccc(-c3ccc(N(c4ccccc4)c4ccc(-c5ccc(-c6ccc(N(c7ccccc7)c7ccc(-c8ccc(-c9ccccc9)s8)cc7)cc6)cc5)cc4)cc3)s2)cc1 ; CC1(C)c2cc(N(c3ccccc3)c3ccc(-c4ccc(-c5ccccc5)s4)cc3)ccc2-c2ccc(N(c3ccccc3)c3ccc(-c4ccc(-c5ccccc5)s4)cc3)cc21]

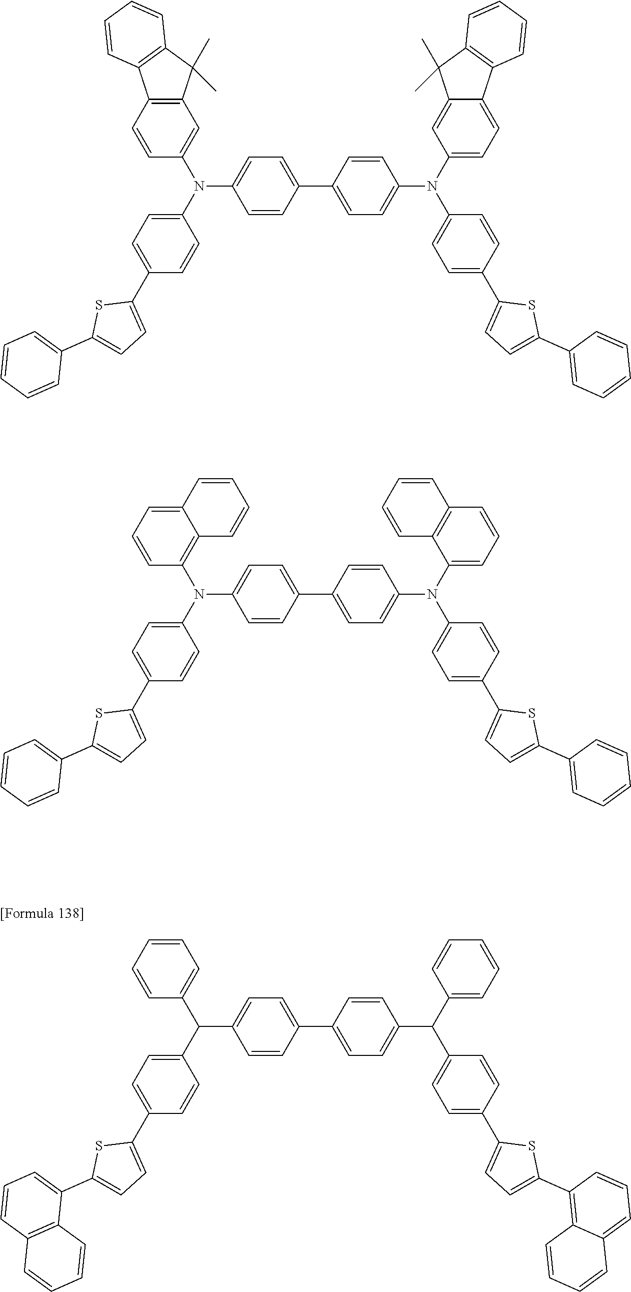 [SMILES: CC1(C)c2ccccc2-c2ccc(N(c3ccc(-c4ccc(N(c5ccc(-c6ccc(-c7ccccc7)s6)cc5)c5ccc6c(c5)C(C)(C)c5ccccc5-6)cc4)cc3)c3ccc(-c4ccc(-c5ccccc5)s4)cc3)cc21 ; c1ccc(-c2ccc(-c3ccc(N(c4ccc(-c5ccc(N(c6ccc(-c7ccc(-c8ccccc8)s7)cc6)c6cccc7ccccc67)cc5)cc4)c4cccc5ccccc45)cc3)s2)cc1 ; c1ccc(C(c2ccc(-c3ccc(C(c4ccccc4)c4ccc(-c5ccc(-c6cccc7ccccc67)s5)cc4)cc3)cc2)c2ccc(-c3ccc(-c4cccc5ccccc45)s3)cc2)cc1]

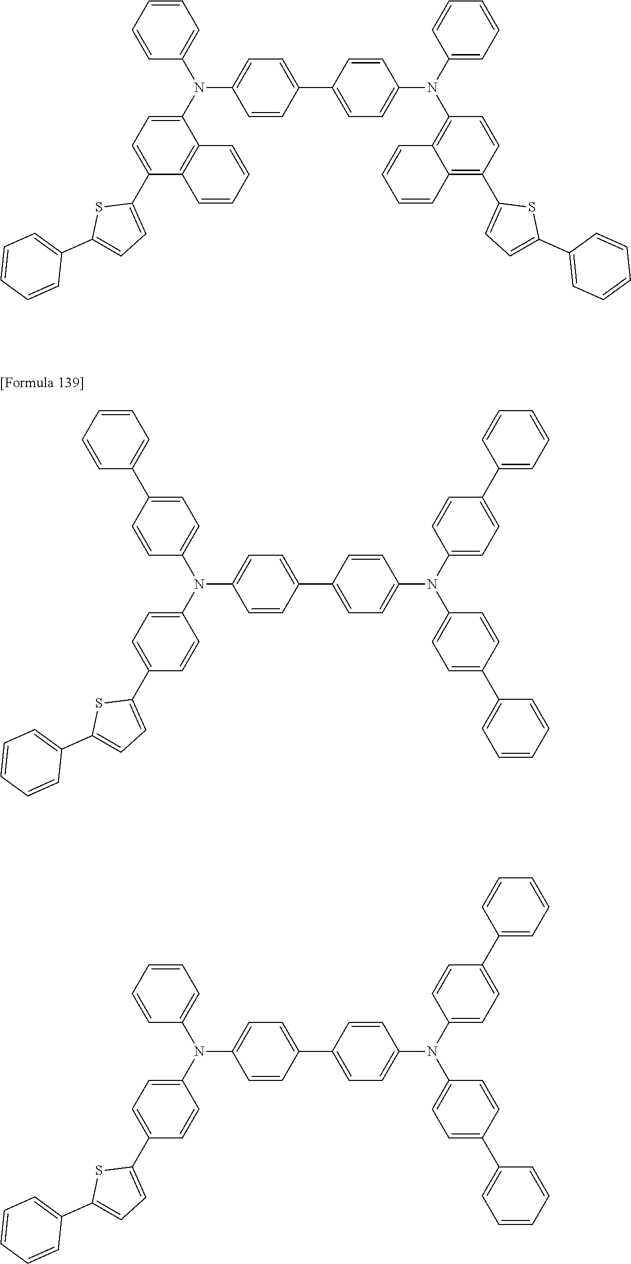 [SMILES: c1ccc(-c2ccc(-c3ccc(N(c4ccccc4)c4ccc(-c5ccc(N(c6ccccc6)c6ccc(-c7ccc(-c8ccccc8)s7)c7ccccc67)cc5)cc4)c4ccccc34)s2)cc1 ; c1ccc(-c2ccc(N(c3ccc(-c4ccccc4)cc3)c3ccc(-c4ccc(N(c5ccc(-c6ccccc6)cc5)c5ccc(-c6ccc(-c7ccccc7)s6)cc5)cc4)cc3)cc2)cc1 ; c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1]

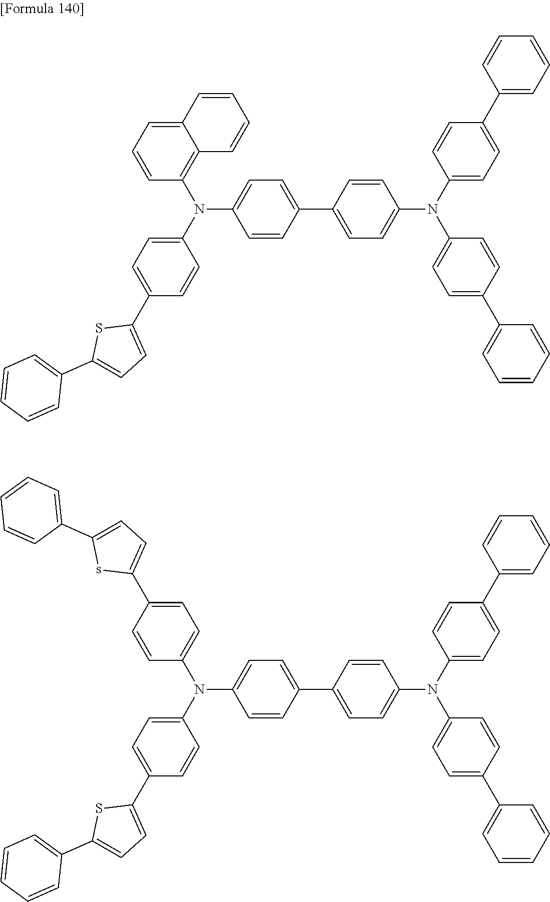

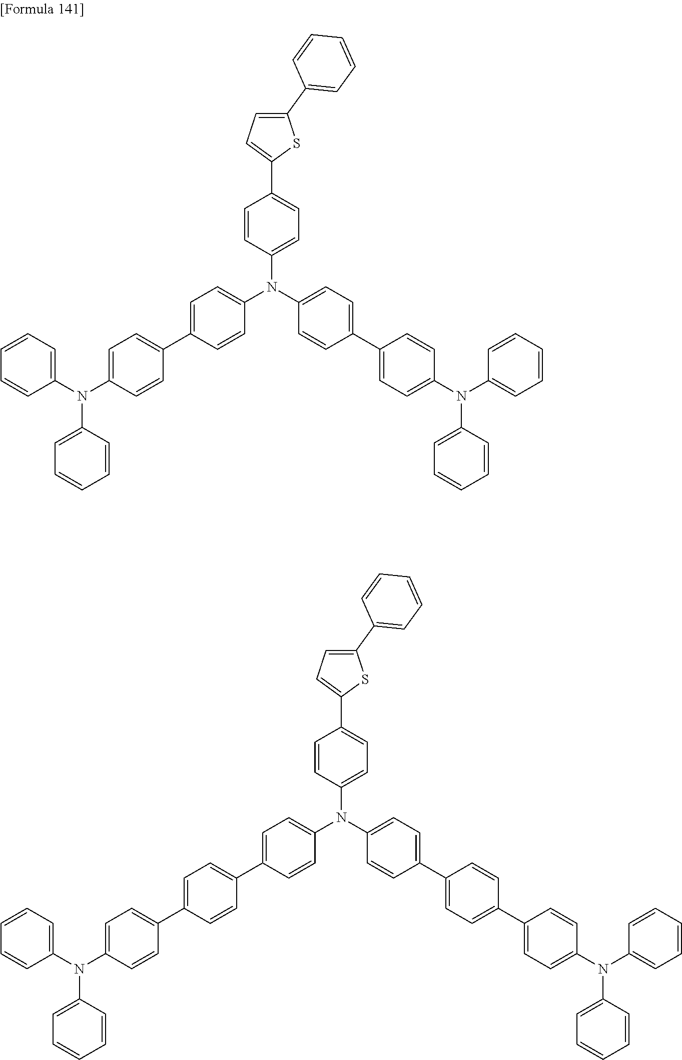 [SMILES: c1ccc(-c2ccc(-c3ccc(N(c4ccc(-c5ccc(N(c6ccccc6)c6ccccc6)cc5)cc4)c4ccc(-c5ccc(N(c6ccccc6)c6ccccc6)cc5)cc4)cc3)s2)cc1 ; c1ccc(-c2ccc(-c3ccc(N(c4ccc(-c5ccc(-c6ccc(N(c7ccccc7)c7ccccc7)cc6)cc5)cc4)c4ccc(-c5ccc(-c6ccc(N(c7ccccc7)c7ccccc7)cc6)cc5)cc4)cc3)s2)cc1]

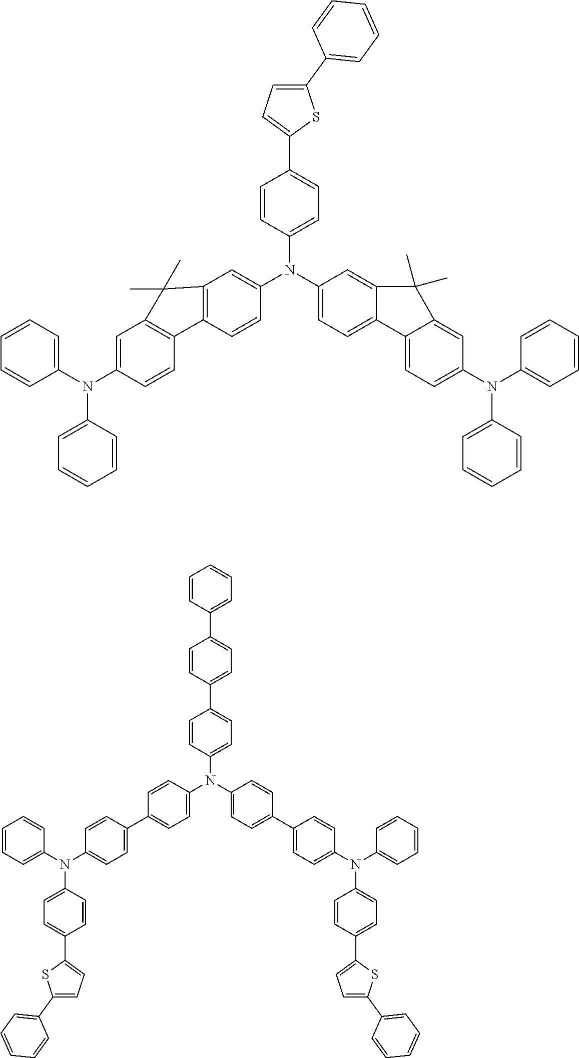 [SMILES: CC1(C)c2cc(N(c3ccccc3)c3ccccc3)ccc2-c2ccc(N(c3ccc(-c4ccc(-c5ccccc5)s4)cc3)c3ccc4c(c3)C(C)(C)c3cc(N(c5ccccc5)c5ccccc5)ccc3-4)cc21 ; c1ccc(-c2ccc(-c3ccc(N(c4ccc(-c5ccc(N(c6ccccc6)c6ccc(-c7ccc(-c8ccccc8)s7)cc6)cc5)cc4)c4ccc(-c5ccc(N(c6ccccc6)c6ccc(-c7ccc(-c8ccccc8)s7)cc6)cc5)cc4)cc3)cc2)cc1]

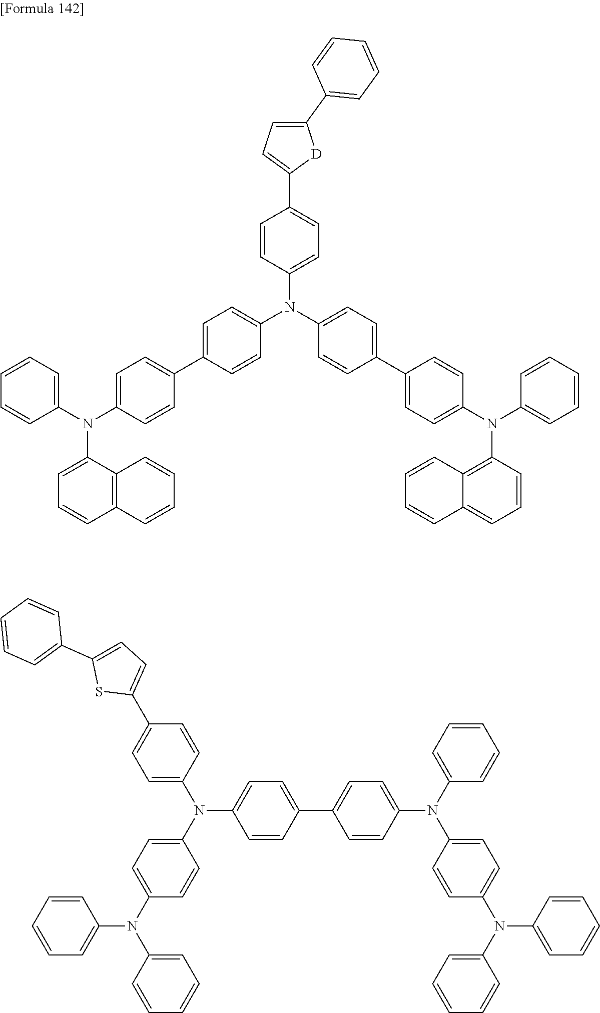

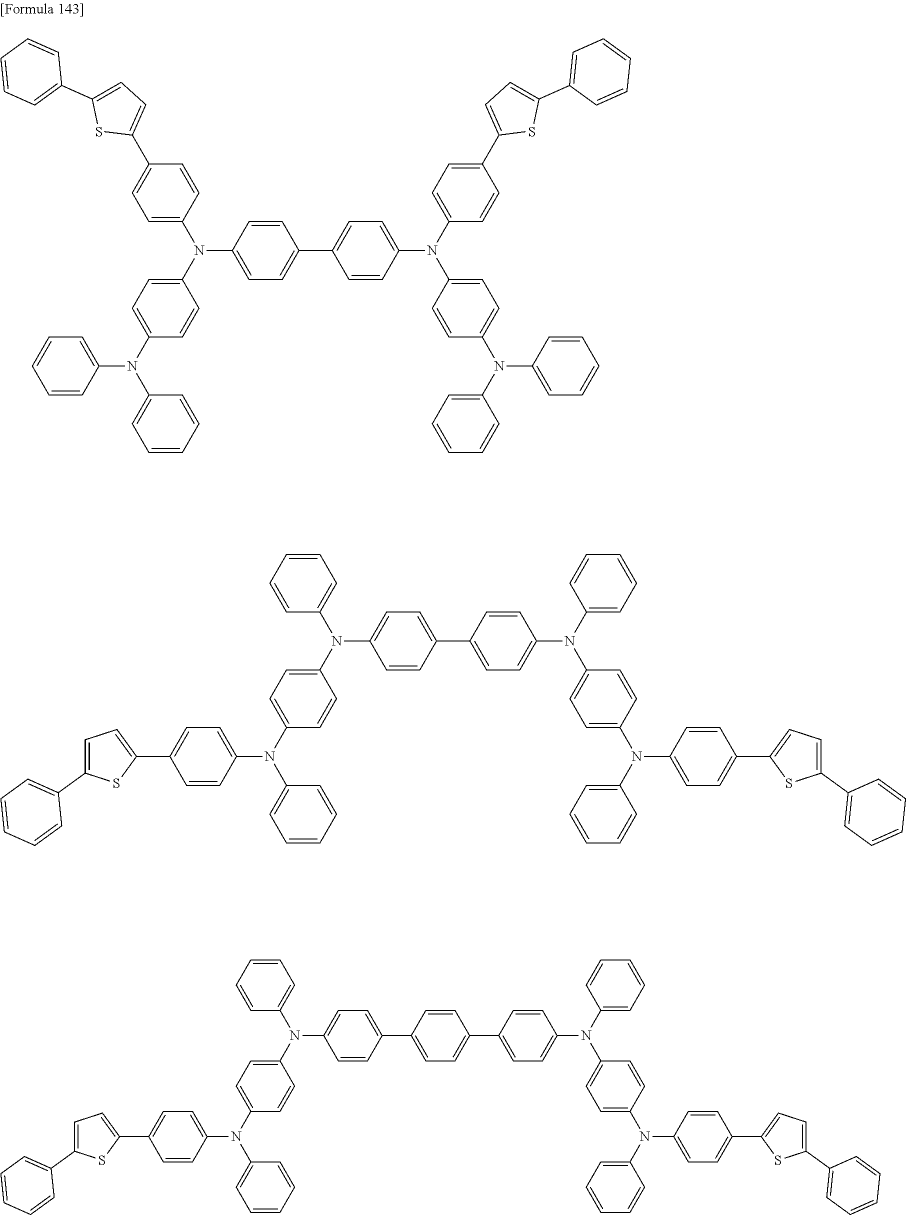 [SMILES: c1ccc(-c2ccc(-c3ccc(N(c4ccc(-c5ccc(N(c6ccc(-c7ccc(-c8ccccc8)s7)cc6)c6ccc(N(c7ccccc7)c7ccccc7)cc6)cc5)cc4)c4ccc(N(c5ccccc5)c5ccccc5)cc4)cc3)s2)cc1 ; c1ccc(-c2ccc(-c3ccc(N(c4ccccc4)c4ccc(N(c5ccccc5)c5ccc(-c6ccc(N(c7ccccc7)c7ccc(N(c8ccccc8)c8ccc(-c9ccc(-c%10ccccc%10)s9)cc8)cc7)cc6)cc5)cc4)cc3)s2)cc1 ; c1ccc(-c2ccc(-c3ccc(N(c4ccccc4)c4ccc(N(c5ccccc5)c5ccc(-c6ccc(-c7ccc(N(c8ccccc8)c8ccc(N(c9ccccc9)c9ccc(-c%10ccc(-c%11ccccc%11)s%10)cc9)cc8)cc7)cc6)cc5)cc4)cc3)s2)cc1]

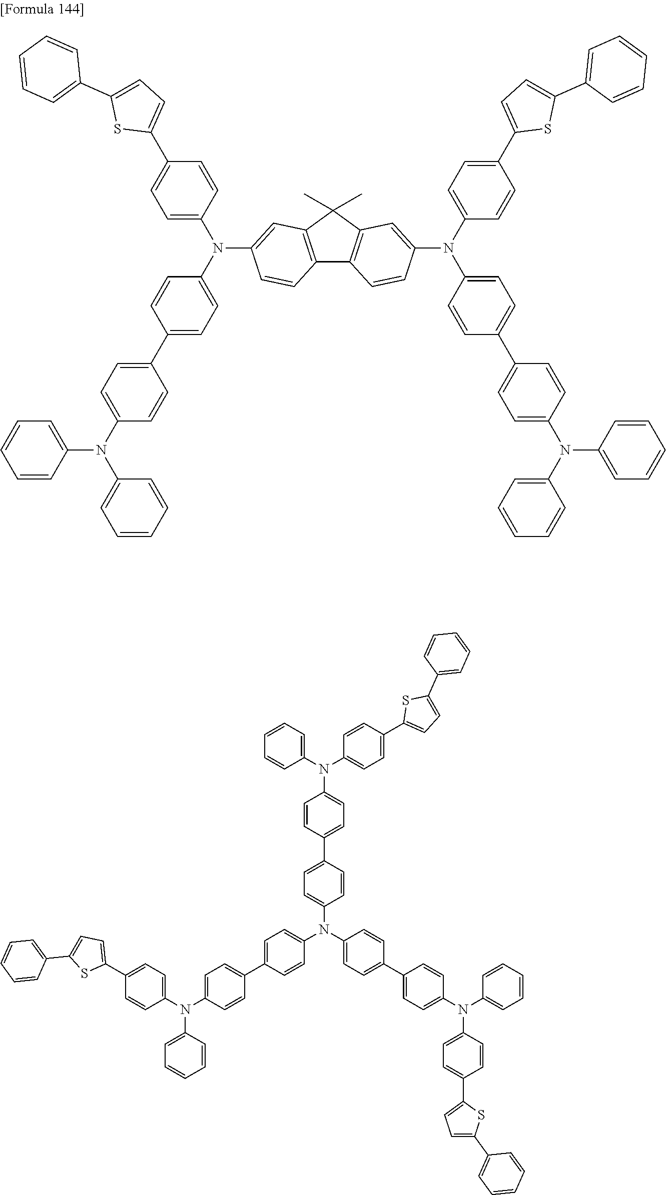 [SMILES: CC1(C)c2cc(N(c3ccc(-c4ccc(N(c5ccccc5)c5ccccc5)cc4)cc3)c3ccc(-c4ccc(-c5ccccc5)s4)cc3)ccc2-c2ccc(N(c3ccc(-c4ccc(N(c5ccccc5)c5ccccc5)cc4)cc3)c3ccc(-c4ccc(-c5ccccc5)s4)cc3)cc21 ; c1ccc(-c2ccc(-c3ccc(N(c4ccccc4)c4ccc(-c5ccc(N(c6ccc(-c7ccc(N(c8ccccc8)c8ccc(-c9ccc(-c%10ccccc%10)s9)cc8)cc7)cc6)c6ccc(-c7ccc(N(c8ccccc8)c8ccc(-c9ccc(-c%10ccccc%10)s9)cc8)cc7)cc6)cc5)cc4)cc3)s2)cc1]

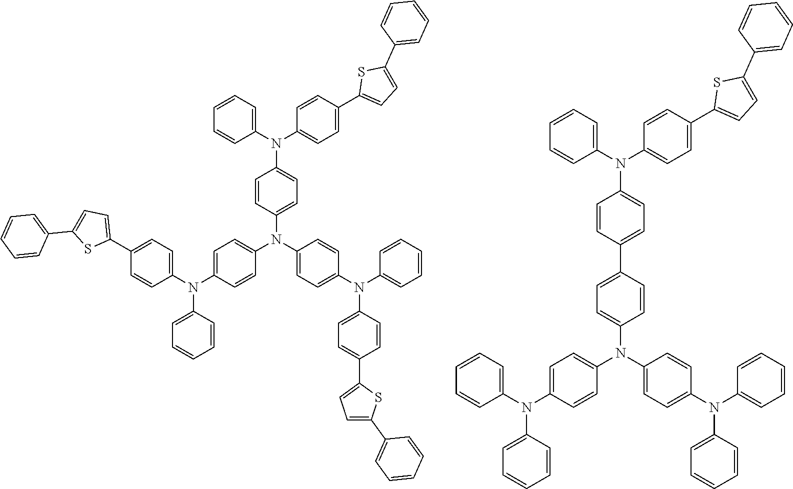 [SMILES: c1ccc(-c2ccc(-c3ccc(N(c4ccccc4)c4ccc(N(c5ccc(N(c6ccccc6)c6ccc(-c7ccc(-c8ccccc8)s7)cc6)cc5)c5ccc(N(c6ccccc6)c6ccc(-c7ccc(-c8ccccc8)s7)cc6)cc5)cc4)cc3)s2)cc1 ; c1ccc(-c2ccc(-c3ccc(N(c4ccccc4)c4ccc(-c5ccc(N(c6ccc(N(c7ccccc7)c7ccccc7)cc6)c6ccc(N(c7ccccc7)c7ccccc7)cc6)cc5)cc4)cc3)s2)cc1]

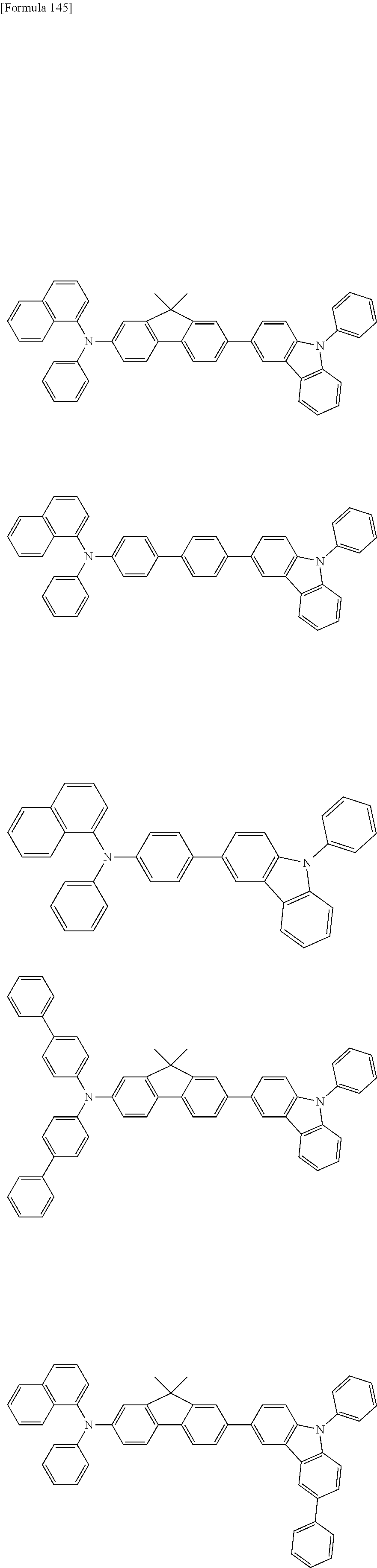 [SMILES: CC1(C)c2cc(-c3ccc4c(c3)c3ccccc3n4-c3ccccc3)ccc2-c2ccc(N(c3ccccc3)c3cccc4ccccc34)cc21 ; c1ccc(N(c2ccc(-c3ccc(-c4ccc5c(c4)c4ccccc4n5-c4ccccc4)cc3)cc2)c2cccc3ccccc23)cc1 ; c1ccc(N(c2ccc(-c3ccc4c(c3)c3ccccc3n4-c3ccccc3)cc2)c2cccc3ccccc23)cc1 ; CC1(C)c2cc(-c3ccc4c(c3)c3ccccc3n4-c3ccccc3)ccc2-c2ccc(N(c3ccc(-c4ccccc4)cc3)c3ccc(-c4ccccc4)cc3)cc21 ; CC1(C)c2cc(-c3ccc4c(c3)c3cc(-c5ccccc5)ccc3n4-c3ccccc3)ccc2-c2ccc(N(c3ccccc3)c3cccc4ccccc34)cc21]

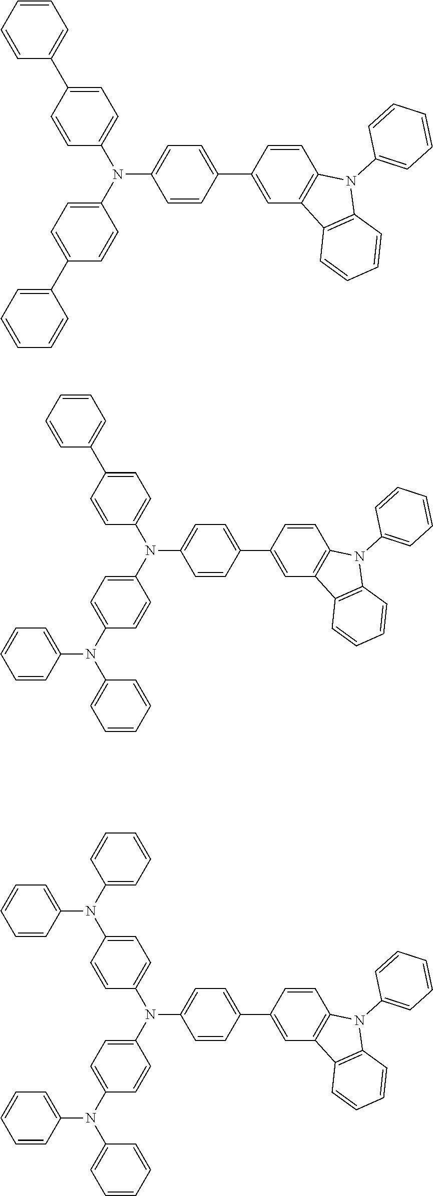 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(N(c3ccc(-c4ccc5c(c4)c4ccccc4n5-c4ccccc4)cc3)c3ccc(N(c4ccccc4)c4ccccc4)cc3)cc2)cc1 ; c1ccc(N(c2ccccc2)c2ccc(N(c3ccc(-c4ccc5c(c4)c4ccccc4n5-c4ccccc4)cc3)c3ccc(N(c4ccccc4)c4ccccc4)cc3)cc2)cc1]

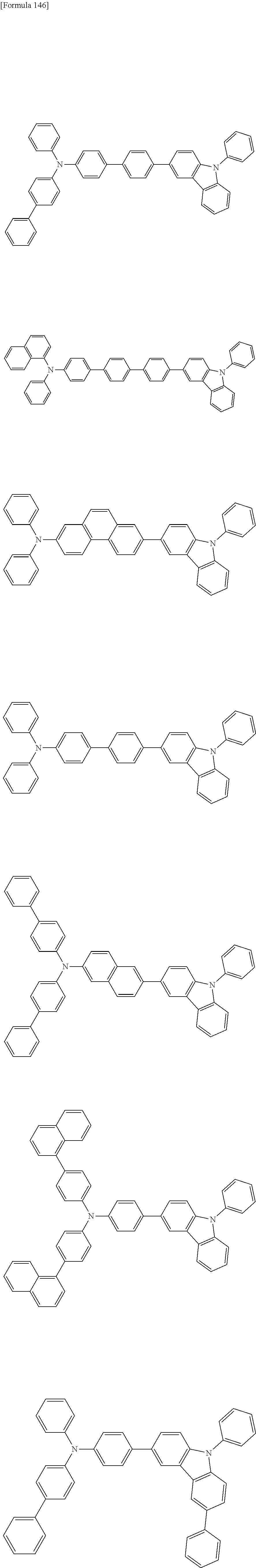

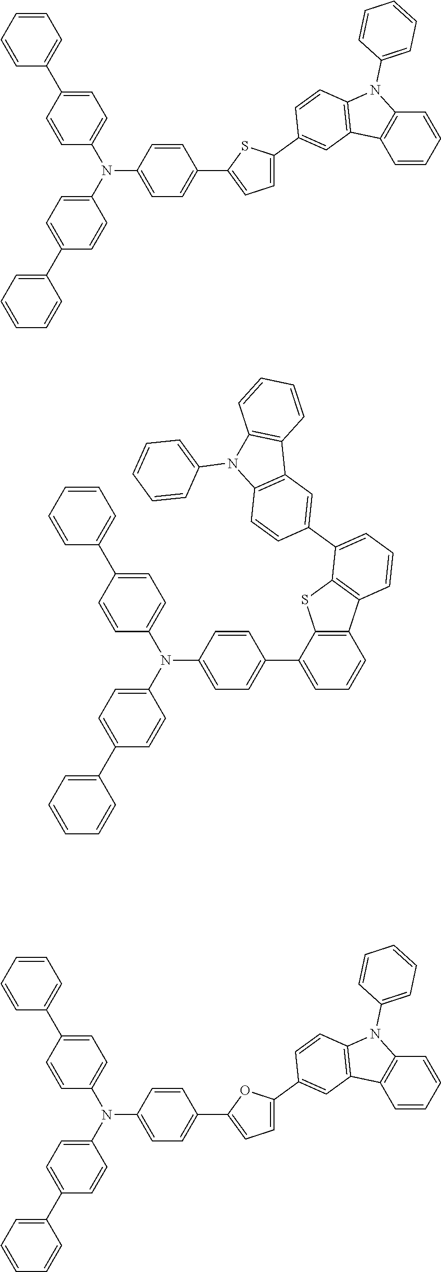 [SMILES: c1ccc(-c2ccc(N(c3ccc(-c4ccccc4)cc3)c3ccc(-c4ccc(-c5ccc6c(c5)c5ccccc5n6-c5ccccc5)s4)cc3)cc2)cc1 ; c1ccc(-c2ccc(N(c3ccc(-c4ccccc4)cc3)c3ccc(-c4cccc5c4sc4c(-c6ccc7c(c6)c6ccccc6n7-c6ccccc6)cccc45)cc3)cc2)cc1 ; c1ccc(-c2ccc(N(c3ccc(-c4ccccc4)cc3)c3ccc(-c4ccc(-c5ccc6c(c5)c5ccccc5n6-c5ccccc5)o4)cc3)cc2)cc1]

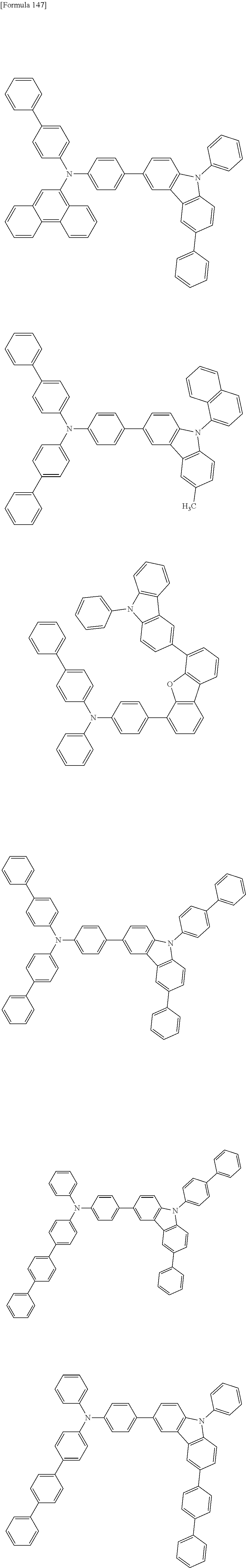

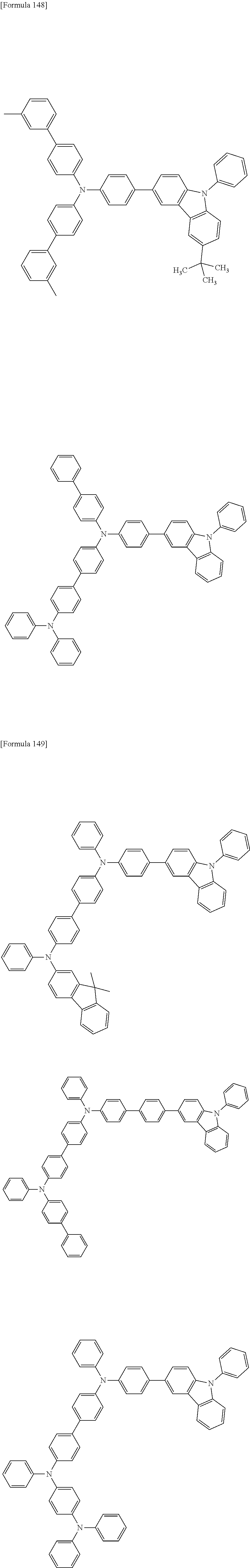

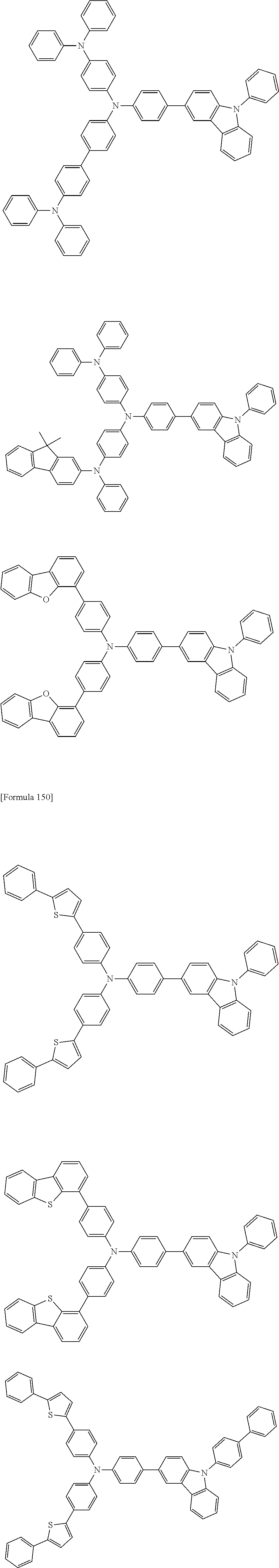

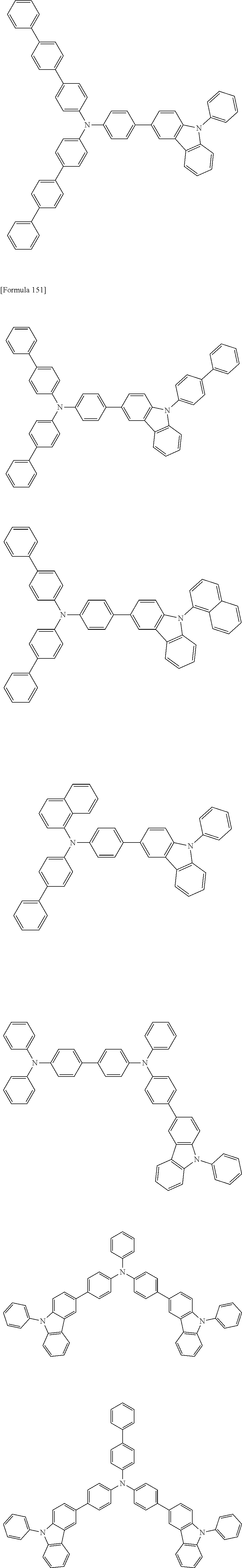

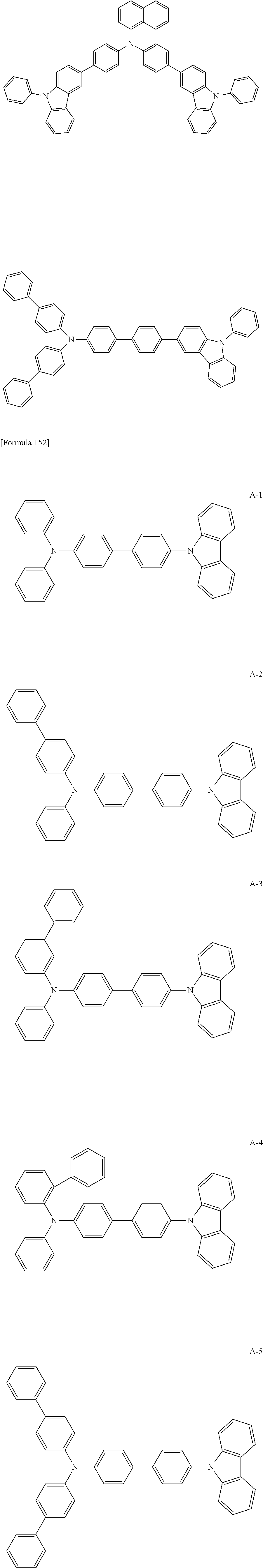

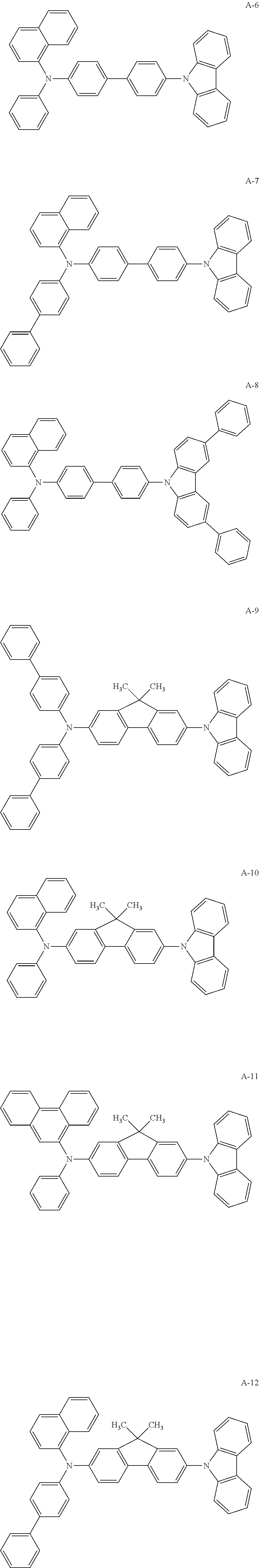

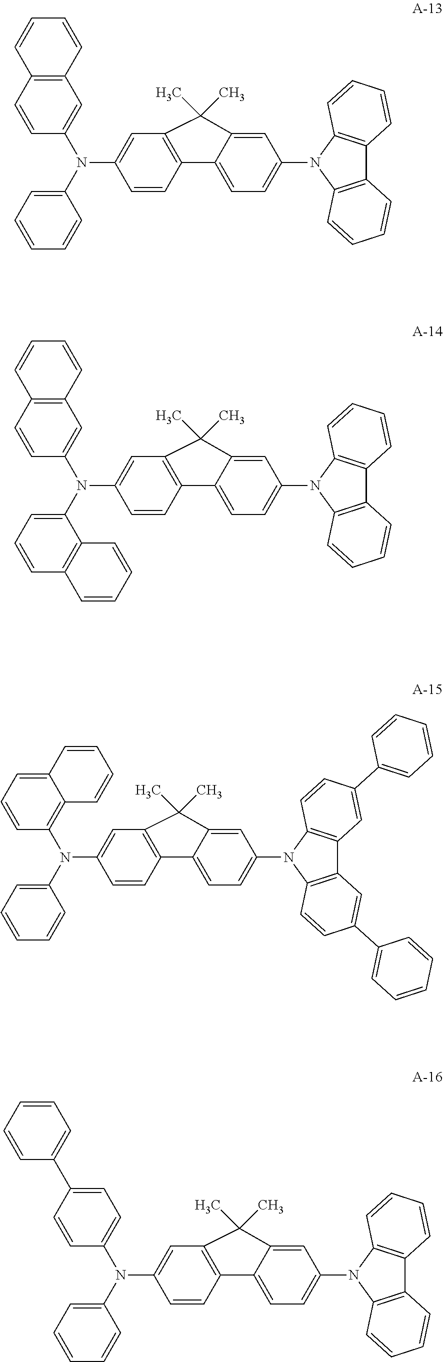 [SMILES: CC1(C)c2cc(N(c3ccccc3)c3ccc4ccccc4c3)ccc2-c2ccc(-n3c4ccccc4c4ccccc43)cc21 ; CC1(C)c2cc(N(c3ccc4ccccc4c3)c3cccc4ccccc34)ccc2-c2ccc(-n3c4ccccc4c4ccccc43)cc21 ; CC1(C)c2cc(N(c3ccccc3)c3cccc4ccccc34)ccc2-c2ccc(-n3c4ccc(-c5ccccc5)cc4c4cc(-c5ccccc5)ccc43)cc21 ; CC1(C)c2cc(N(c3ccccc3)c3ccc(-c4ccccc4)cc3)ccc2-c2ccc(-n3c4ccccc4c4ccccc43)cc21]

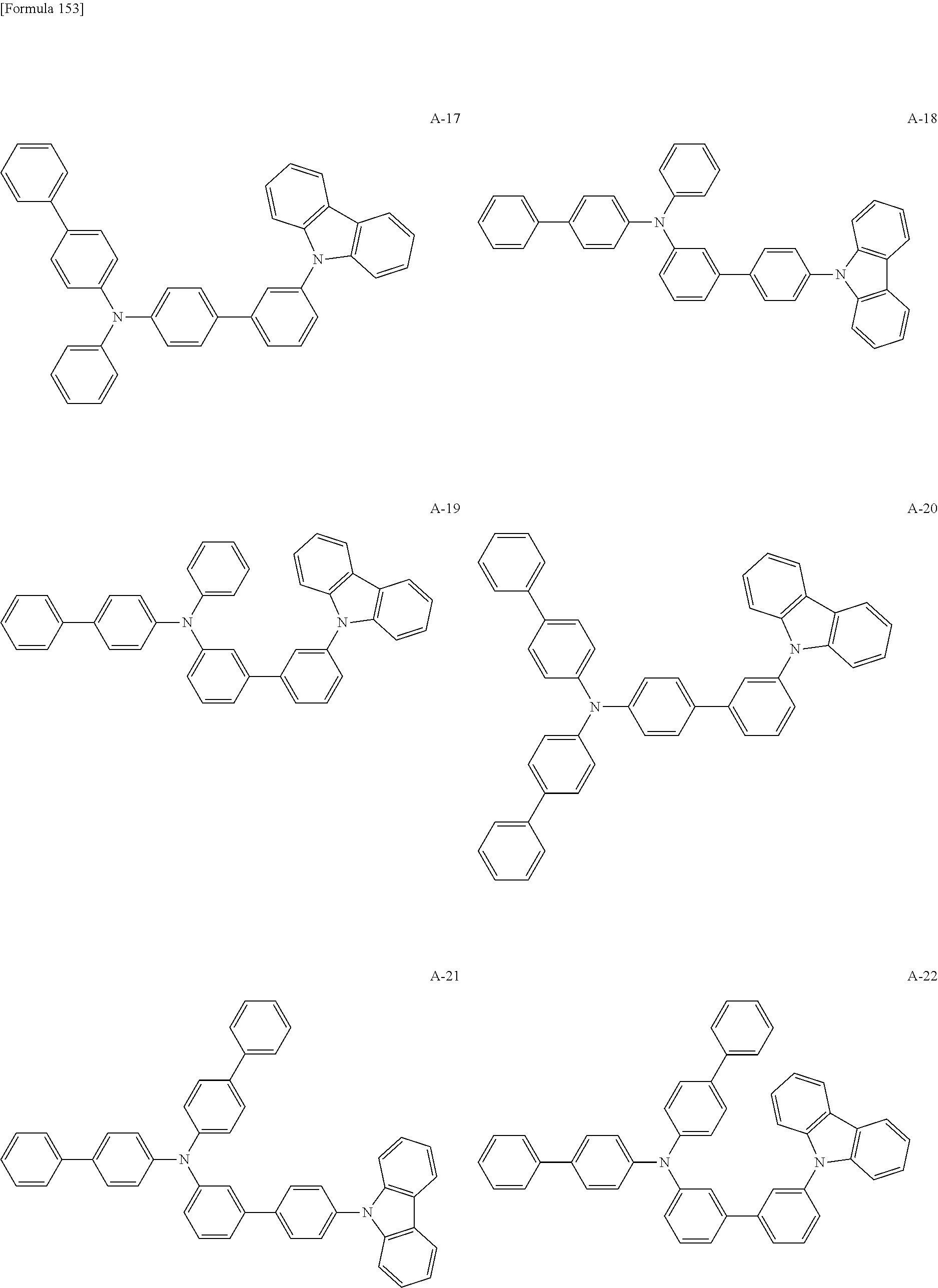 [SMILES: c1ccc(-c2ccc(N(c3ccccc3)c3ccc(-c4cccc(-n5c6ccccc6c6ccccc65)c4)cc3)cc2)cc1 ; c1ccc(-c2ccc(N(c3ccccc3)c3cccc(-c4ccc(-n5c6ccccc6c6ccccc65)cc4)c3)cc2)cc1 ; c1ccc(-c2ccc(N(c3ccccc3)c3cccc(-c4cccc(-n5c6ccccc6c6ccccc65)c4)c3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4)cc3)c3cccc(-c4ccc(-n5c6ccccc6c6ccccc65)cc4)c3)cc2)cc1 ; c1ccc(-c2ccc(N(c3ccc(-c4ccccc4)cc3)c3cccc(-c4cccc(-n5c6ccccc6c6ccccc65)c4)c3)cc2)cc1]

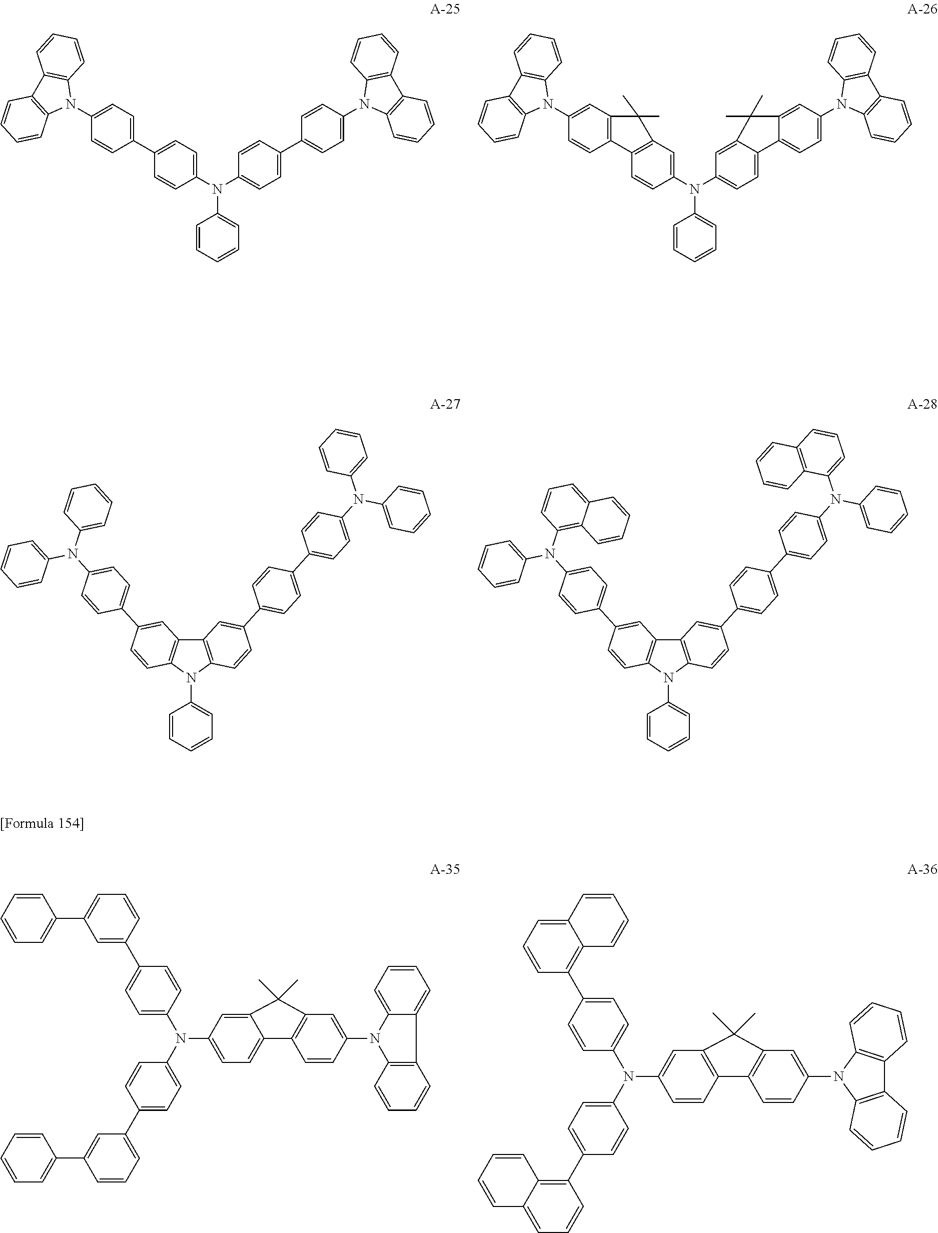

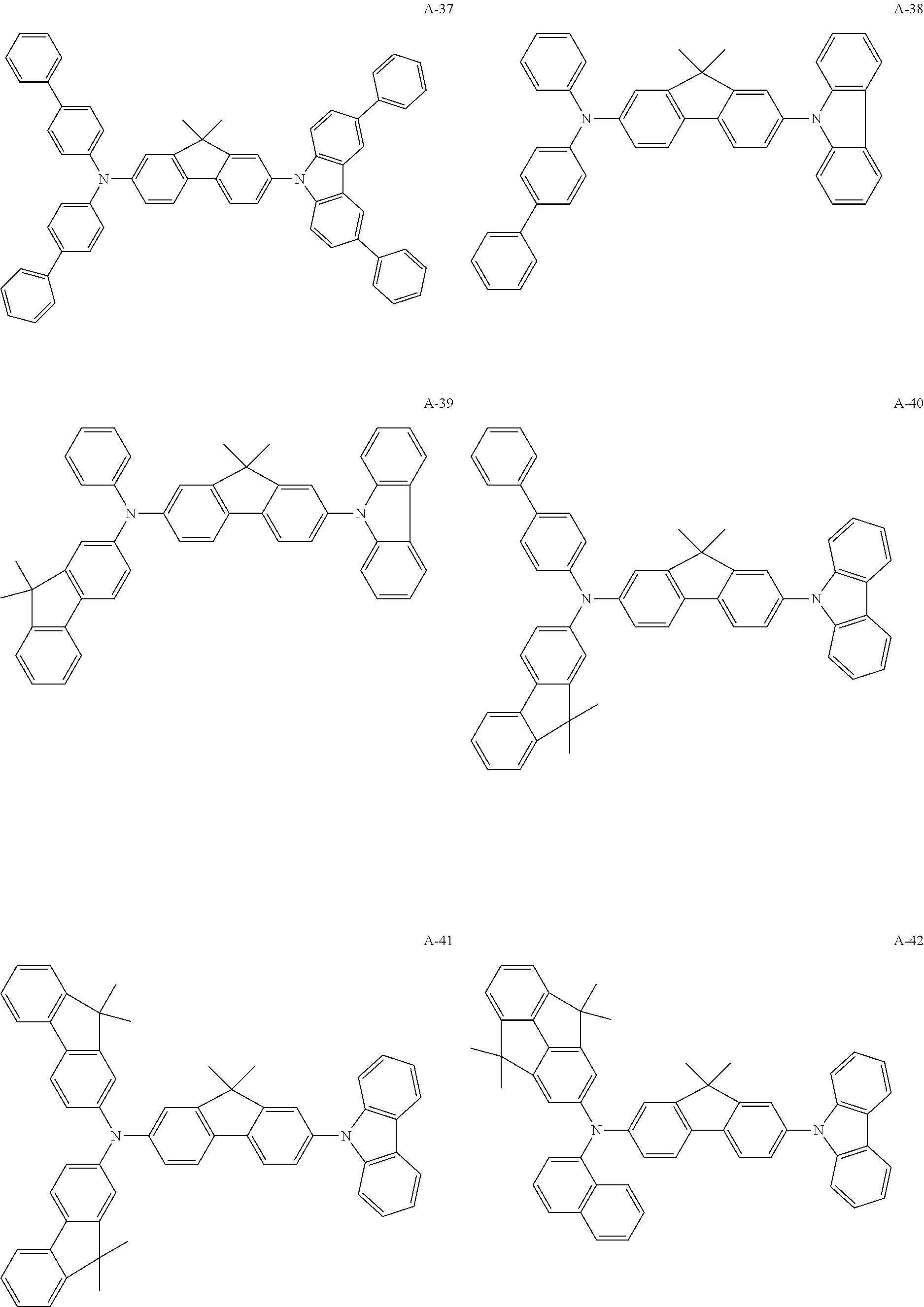

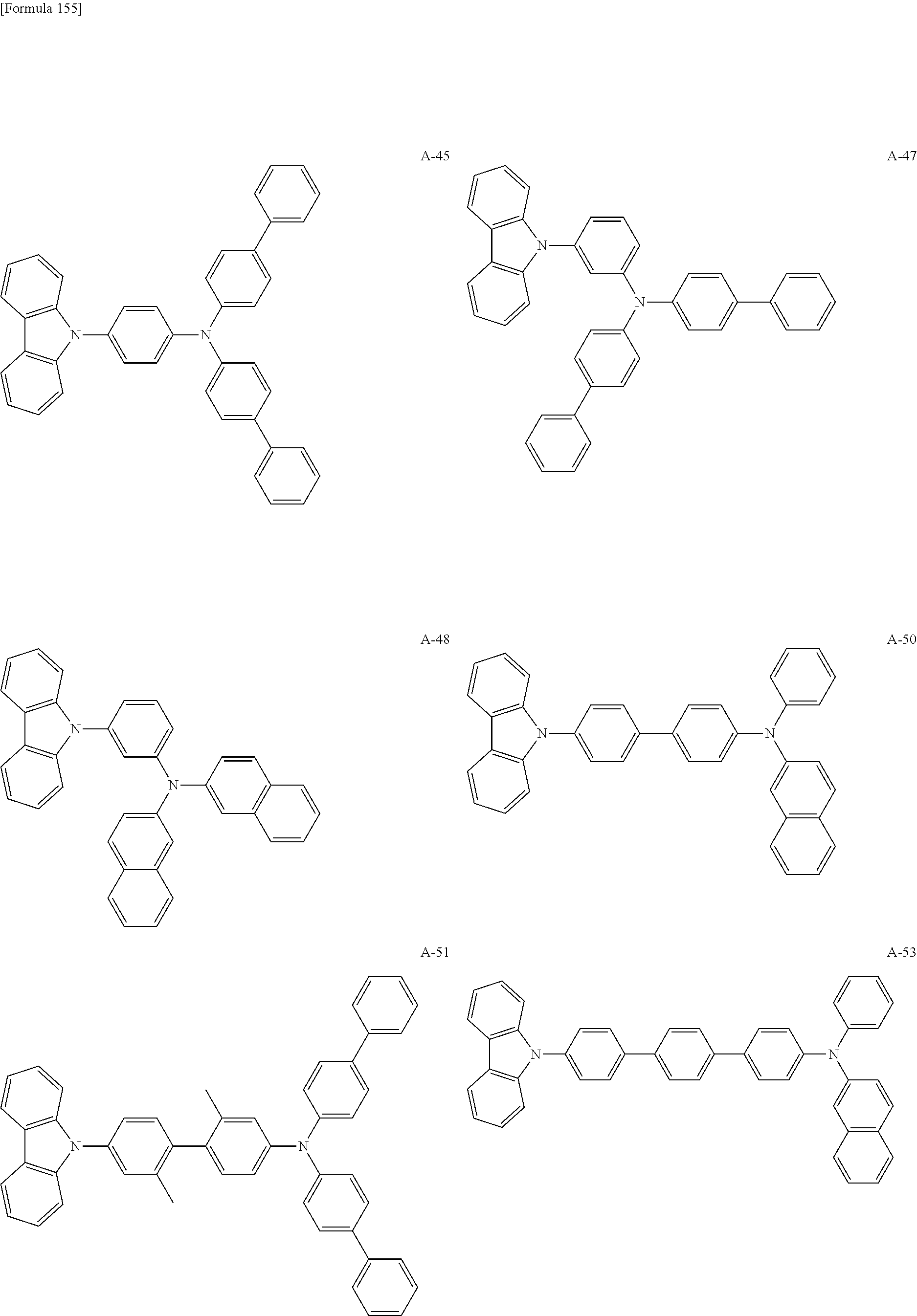 [SMILES: c1ccc(-c2ccc(N(c3ccc(-c4ccccc4)cc3)c3ccc(-n4c5ccccc5c5ccccc54)cc3)cc2)cc1 ; c1ccc(-c2ccc(N(c3ccc(-c4ccccc4)cc3)c3cccc(-n4c5ccccc5c5ccccc54)c3)cc2)cc1 ; c1cc(N(c2ccc3ccccc3c2)c2ccc3ccccc3c2)cc(-n2c3ccccc3c3ccccc32)c1 ; c1ccc(N(c2ccc(-c3ccc(-n4c5ccccc5c5ccccc54)cc3)cc2)c2ccc3ccccc3c2)cc1 ; Cc1cc(N(c2ccc(-c3ccccc3)cc2)c2ccc(-c3ccccc3)cc2)ccc1-c1ccc(-n2c3ccccc3c3ccccc32)cc1C ; c1ccc(N(c2ccc(-c3ccc(-c4ccc(-n5c6ccccc6c6ccccc65)cc4)cc3)cc2)c2ccc3ccccc3c2)cc1]

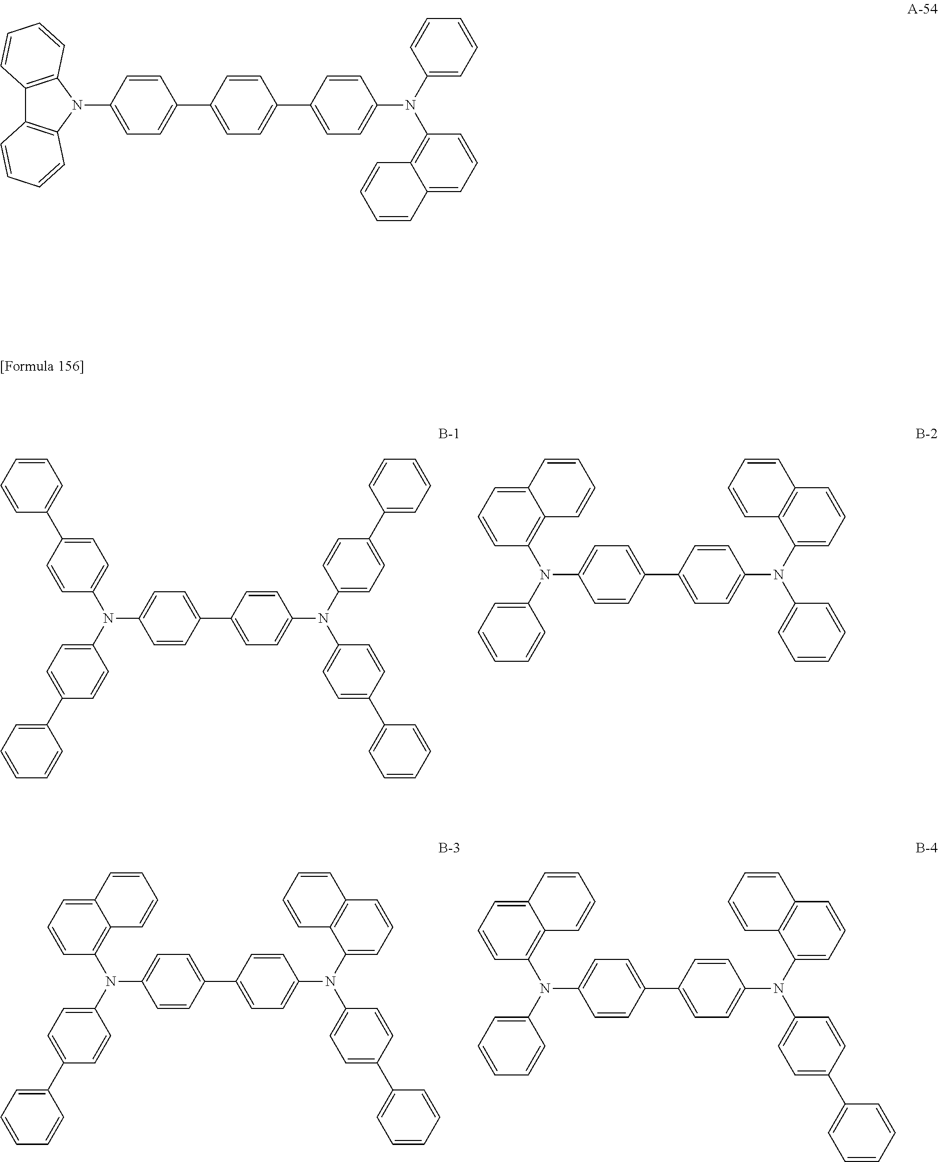 [SMILES: c1ccc(N(c2ccc(-c3ccc(-c4ccc(-n5c6ccccc6c6ccccc65)cc4)cc3)cc2)c2cccc3ccccc23)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccc(N(c5ccc(-c6ccccc6)cc5)c5cccc6ccccc56)cc4)cc3)c3cccc4ccccc34)cc2)cc1 ; c1ccc(-c2ccc(N(c3ccc(-c4ccc(N(c5ccccc5)c5cccc6ccccc56)cc4)cc3)c3cccc4ccccc34)cc2)cc1]

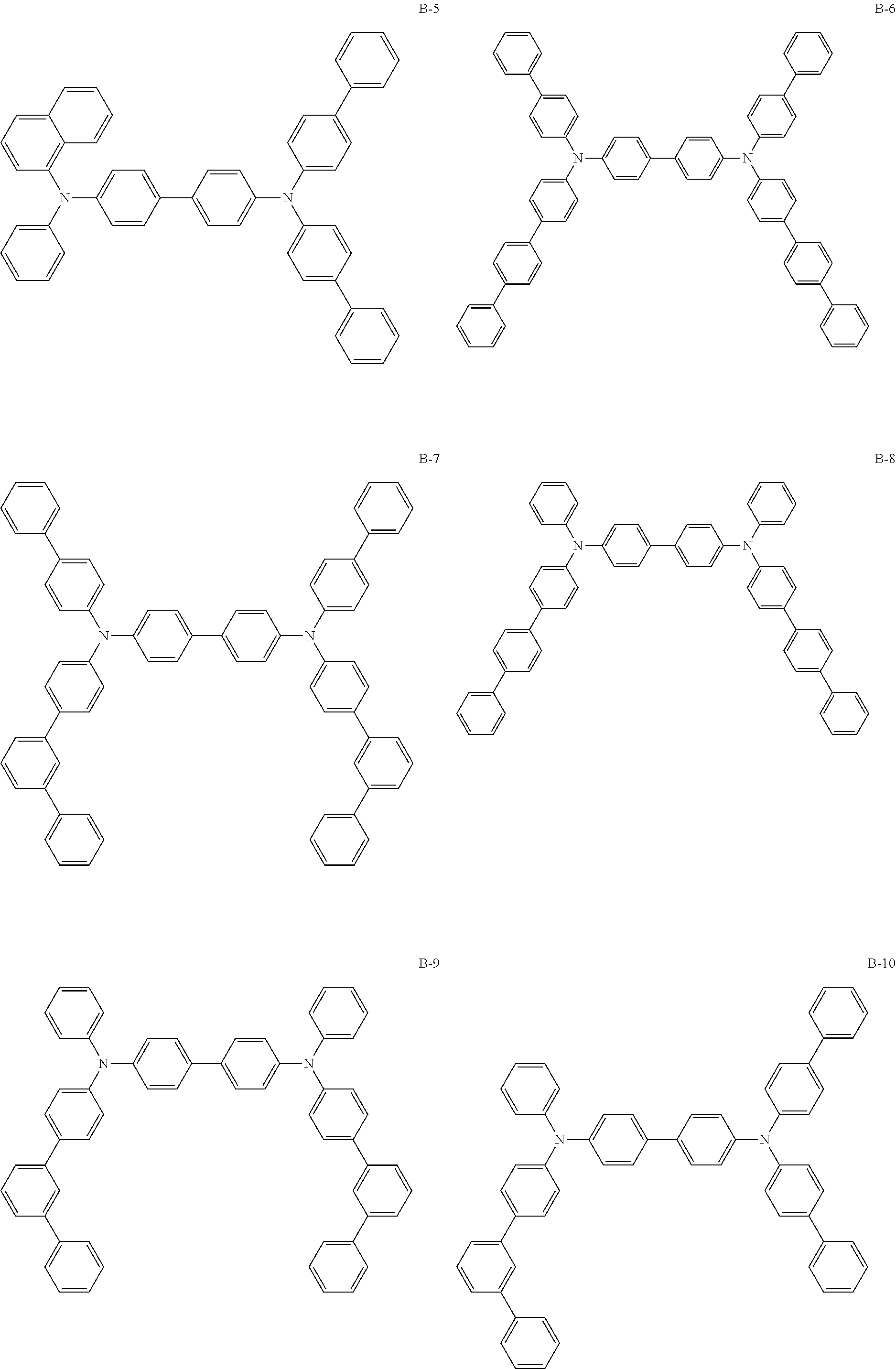

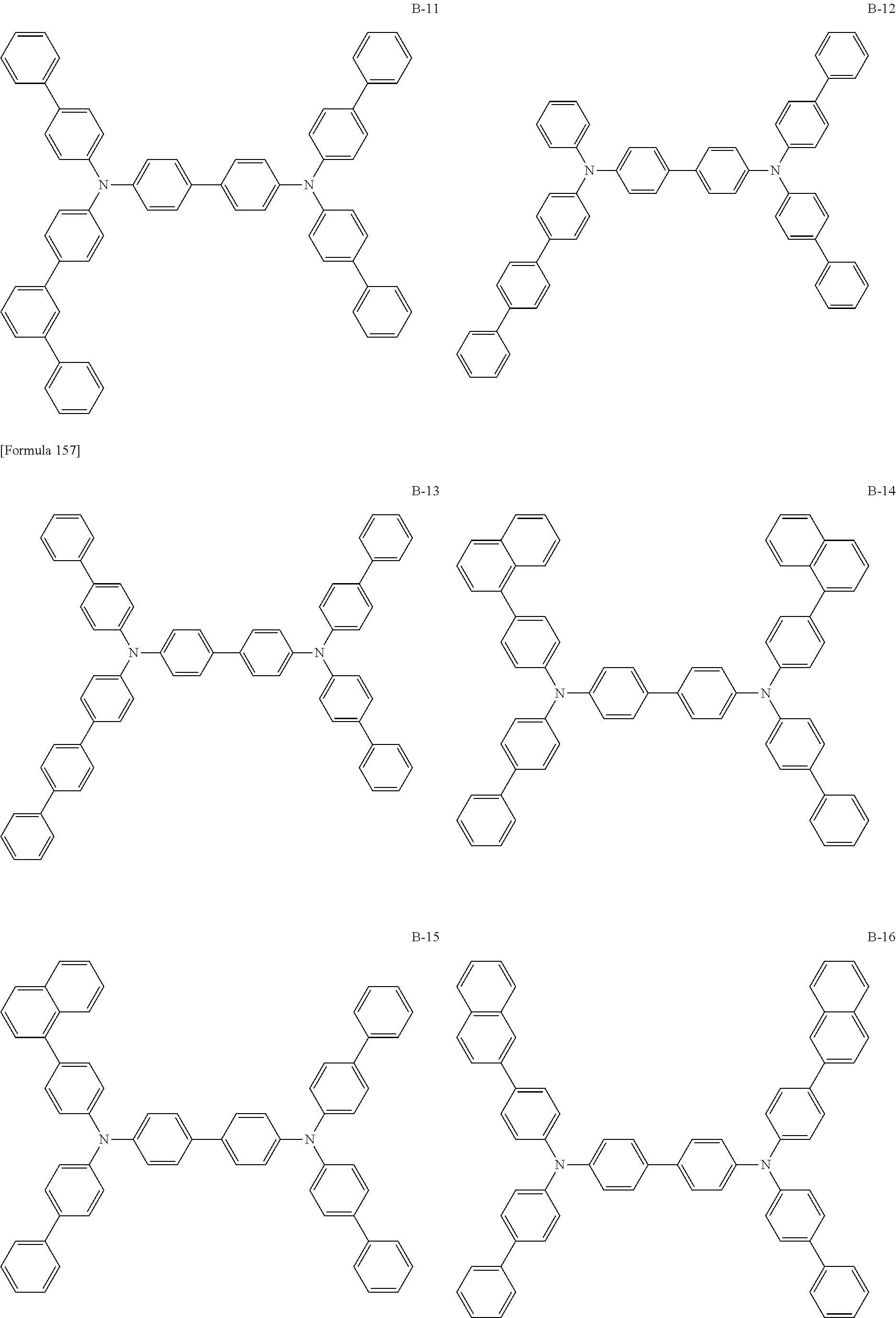

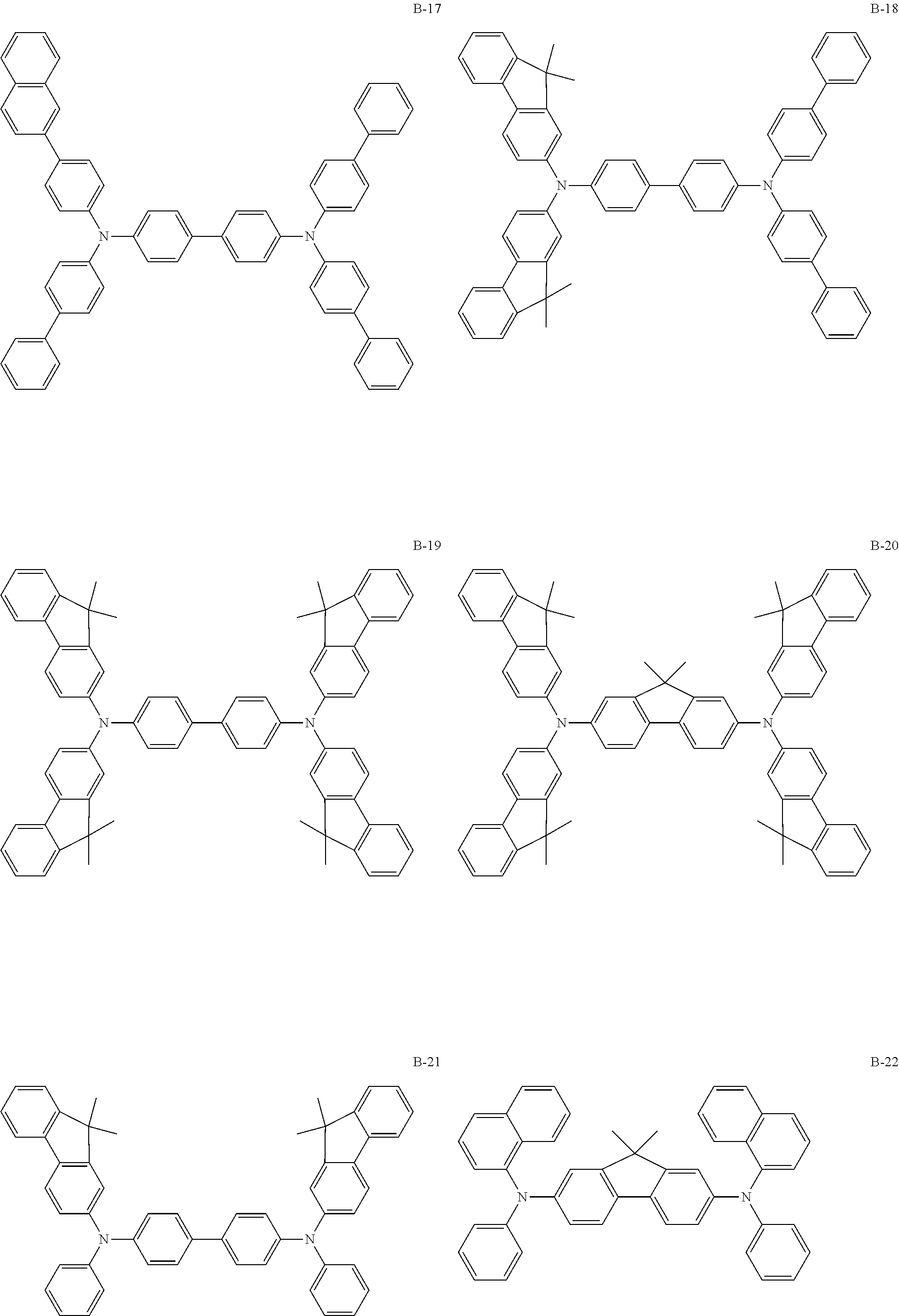

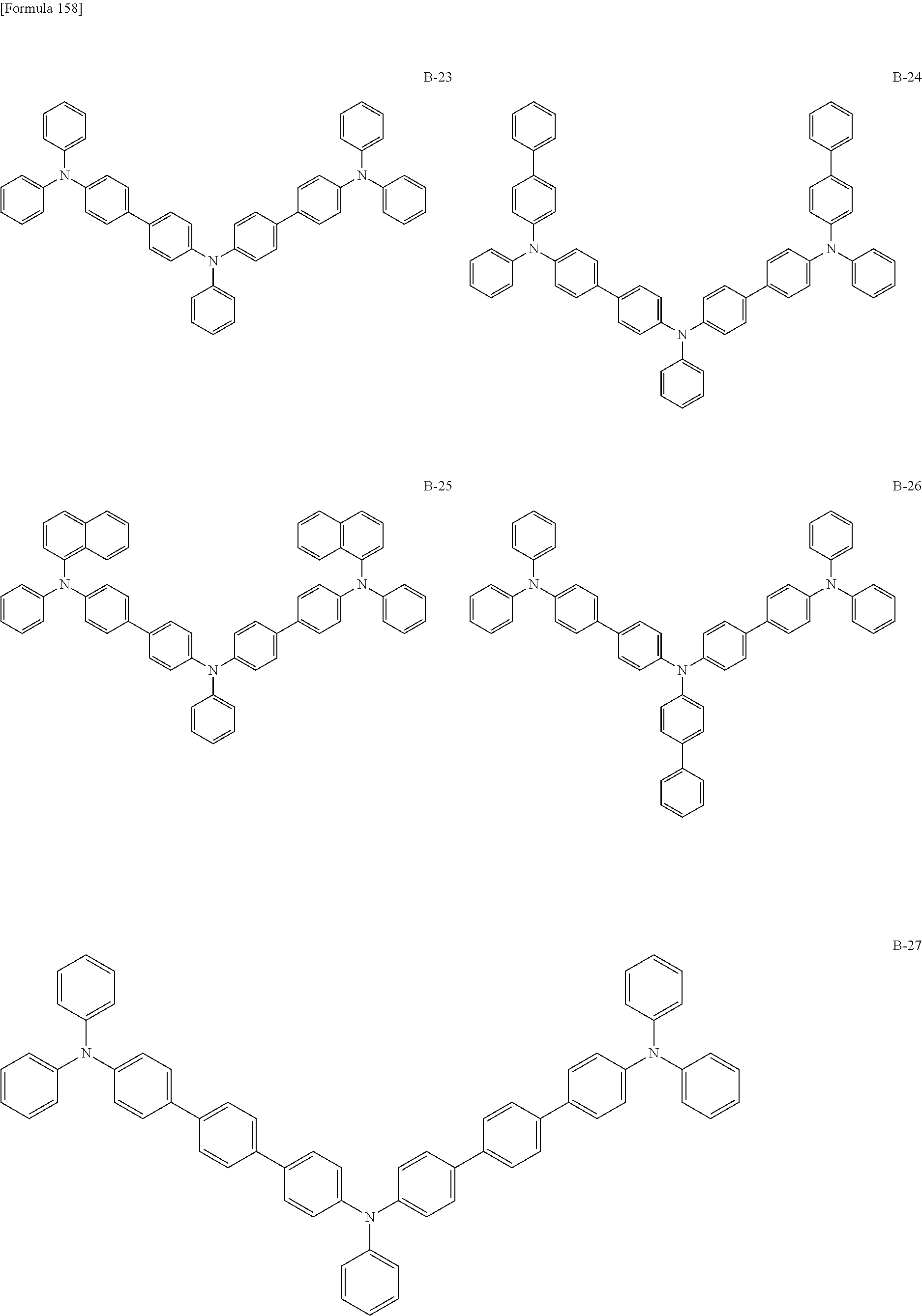

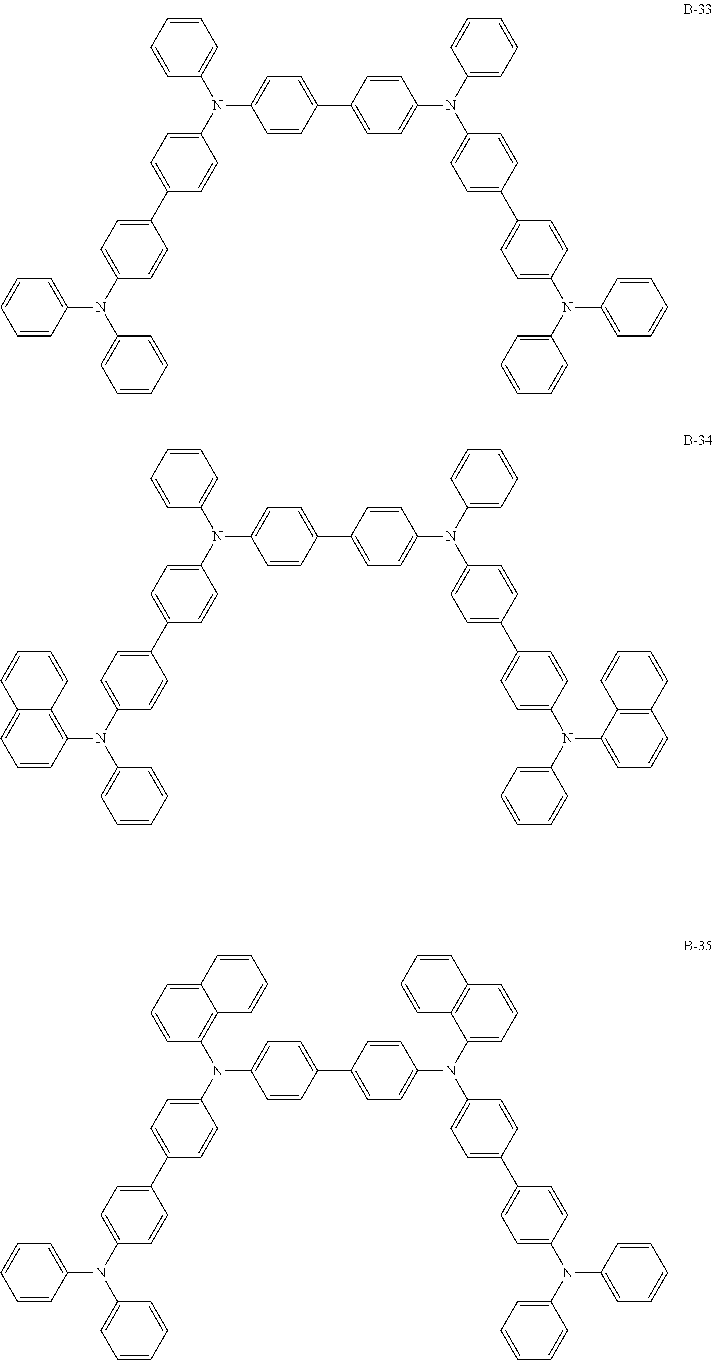 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(N(c2ccc(-c3ccc(N(c4ccccc4)c4ccc(-c5ccc(N(c6ccccc6)c6cccc7ccccc67)cc5)cc4)cc3)cc2)c2ccc(-c3ccc(N(c4ccccc4)c4cccc5ccccc45)cc3)cc2)cc1 ; c1ccc(N(c2ccccc2)c2ccc(-c3ccc(N(c4ccc(-c5ccc(N(c6ccc(-c7ccc(N(c8ccccc8)c8ccccc8)cc7)cc6)c6cccc7ccccc67)cc5)cc4)c4cccc5ccccc45)cc3)cc2)cc1]

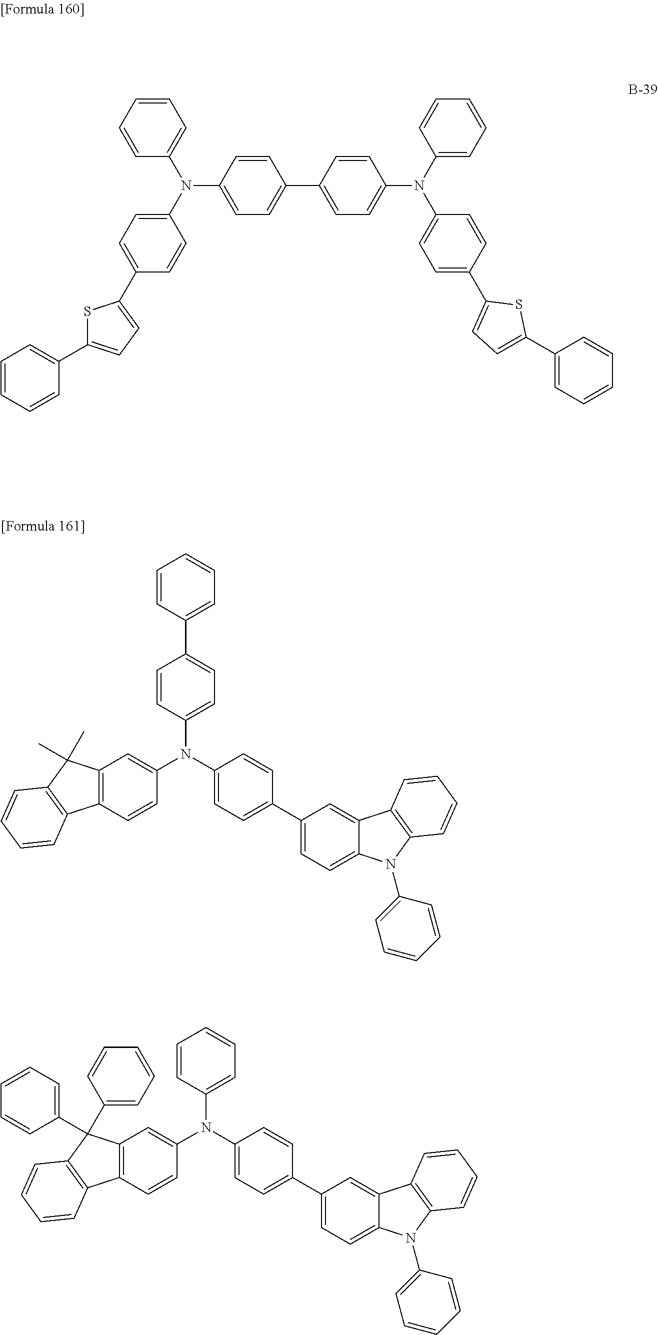 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

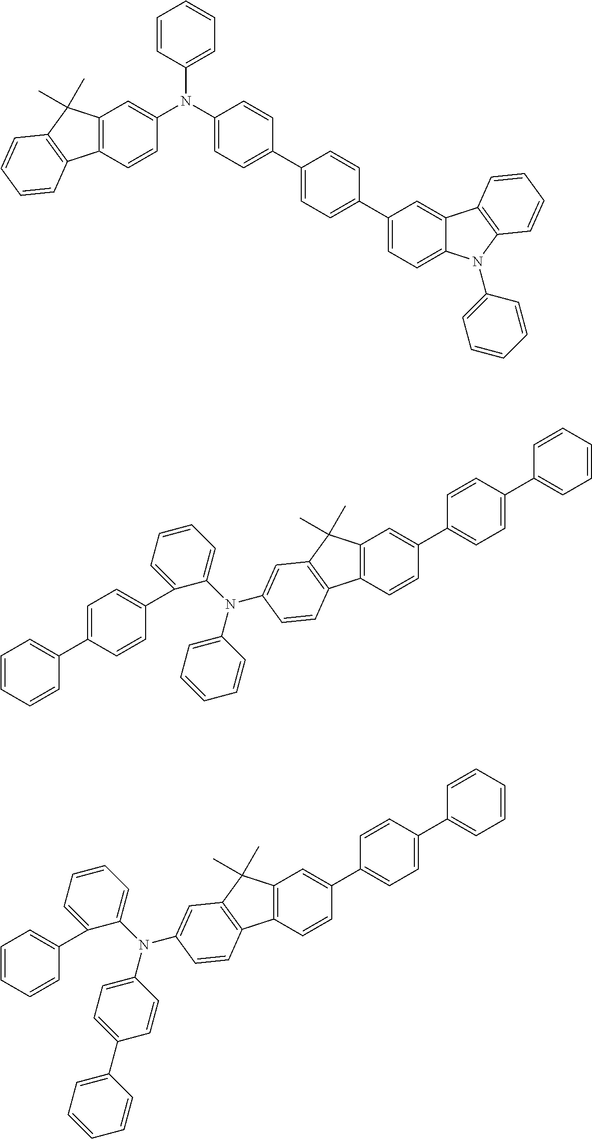 [SMILES: CC1(C)c2ccccc2-c2ccc(N(c3ccccc3)c3ccc(-c4ccc(-c5ccc6c(c5)c5ccccc5n6-c5ccccc5)cc4)cc3)cc21 ; CC1(C)c2cc(-c3ccc(-c4ccccc4)cc3)ccc2-c2ccc(N(c3ccccc3)c3ccccc3-c3ccc(-c4ccccc4)cc3)cc21 ; CC1(C)c2cc(-c3ccc(-c4ccccc4)cc3)ccc2-c2ccc(N(c3ccc(-c4ccccc4)cc3)c3ccccc3-c3ccccc3)cc21]

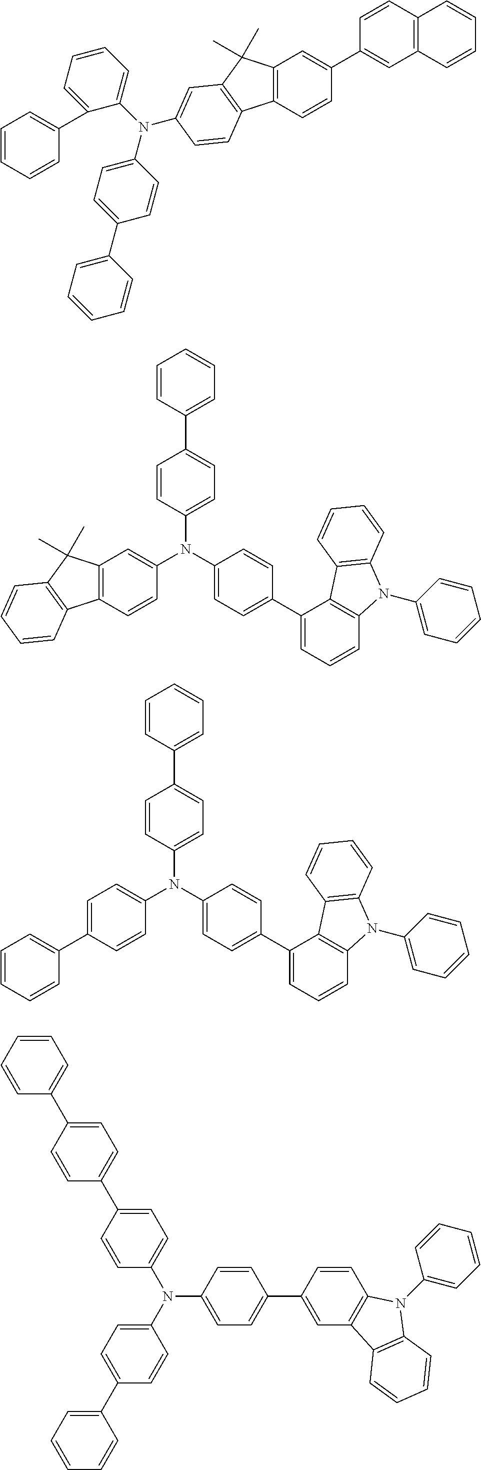

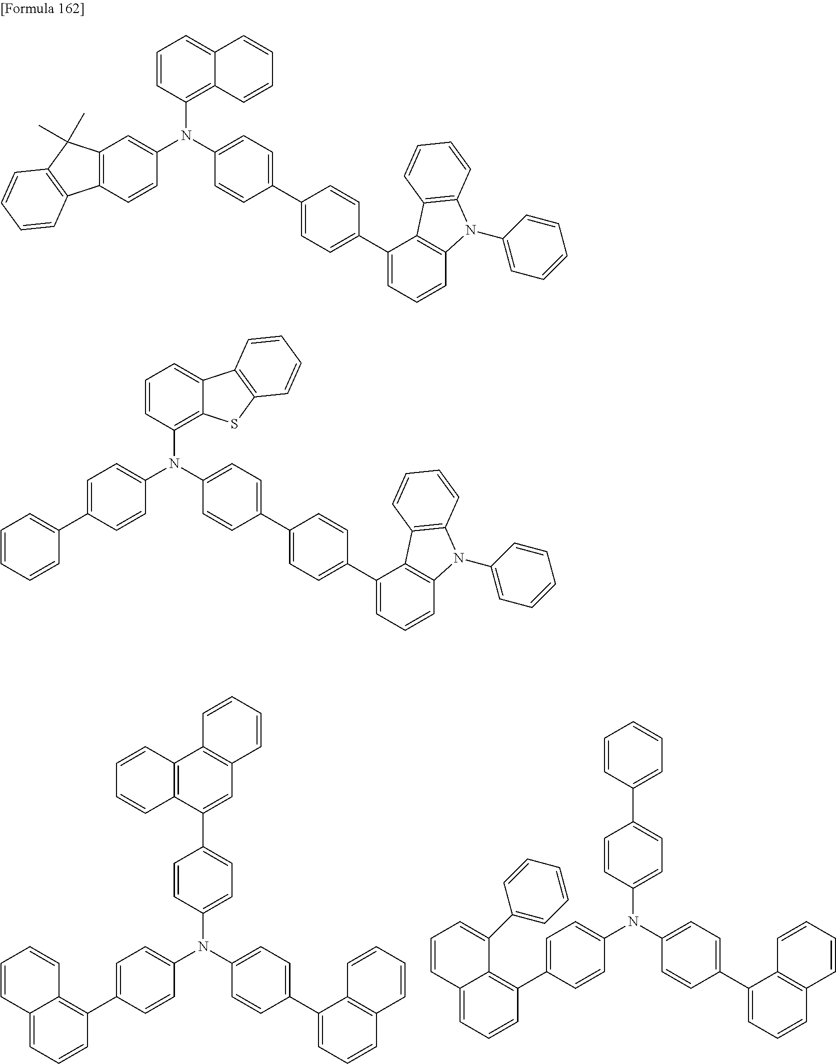

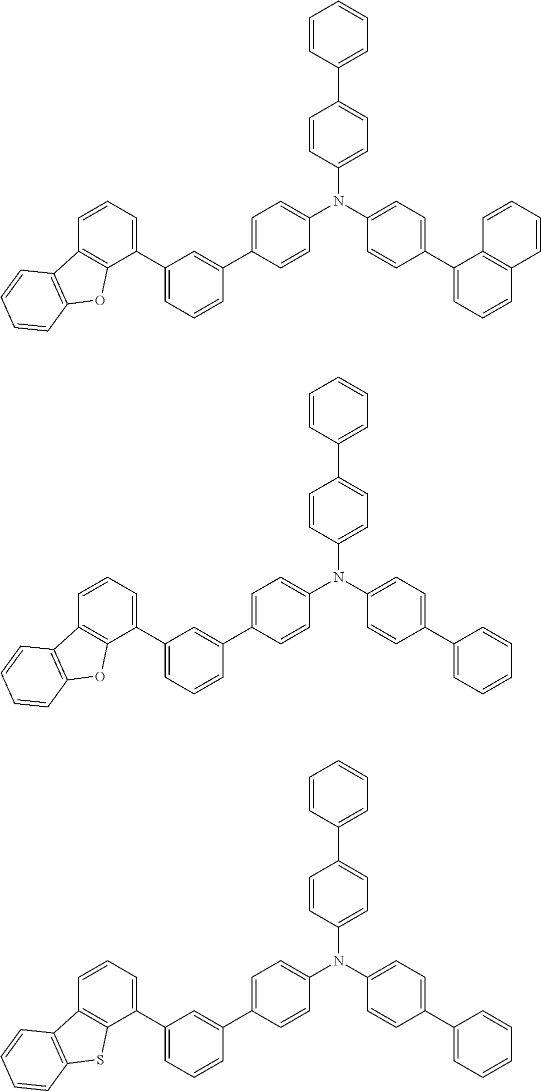 [SMILES: c1ccc(-c2ccc(N(c3ccc(-c4cccc(-c5cccc6c5oc5ccccc56)c4)cc3)c3ccc(-c4cccc5ccccc45)cc3)cc2)cc1 ; c1ccc(-c2ccc(N(c3ccc(-c4ccccc4)cc3)c3ccc(-c4cccc(-c5cccc6c5oc5ccccc56)c4)cc3)cc2)cc1 ; c1ccc(-c2ccc(N(c3ccc(-c4ccccc4)cc3)c3ccc(-c4cccc(-c5cccc6c5sc5ccccc56)c4)cc3)cc2)cc1]

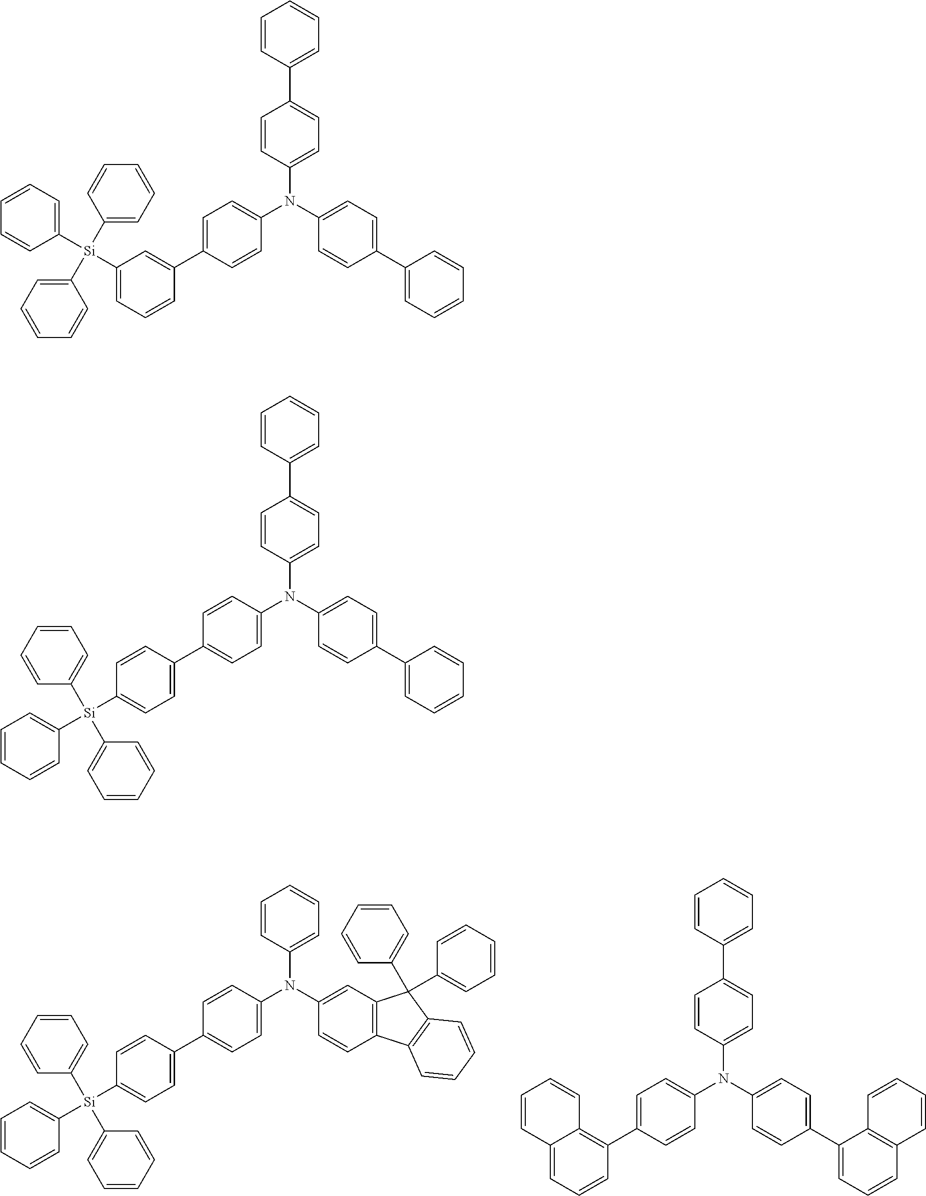 [SMILES: c1ccc(-c2ccc(N(c3ccc(-c4ccccc4)cc3)c3ccc(-c4cccc([Si](c5ccccc5)(c5ccccc5)c5ccccc5)c4)cc3)cc2)cc1 ; c1ccc(-c2ccc(N(c3ccc(-c4ccccc4)cc3)c3ccc(-c4ccc([Si](c5ccccc5)(c5ccccc5)c5ccccc5)cc4)cc3)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; c1ccc(-c2ccc(N(c3ccc(-c4cccc5ccccc45)cc3)c3ccc(-c4cccc5ccccc45)cc3)cc2)cc1]

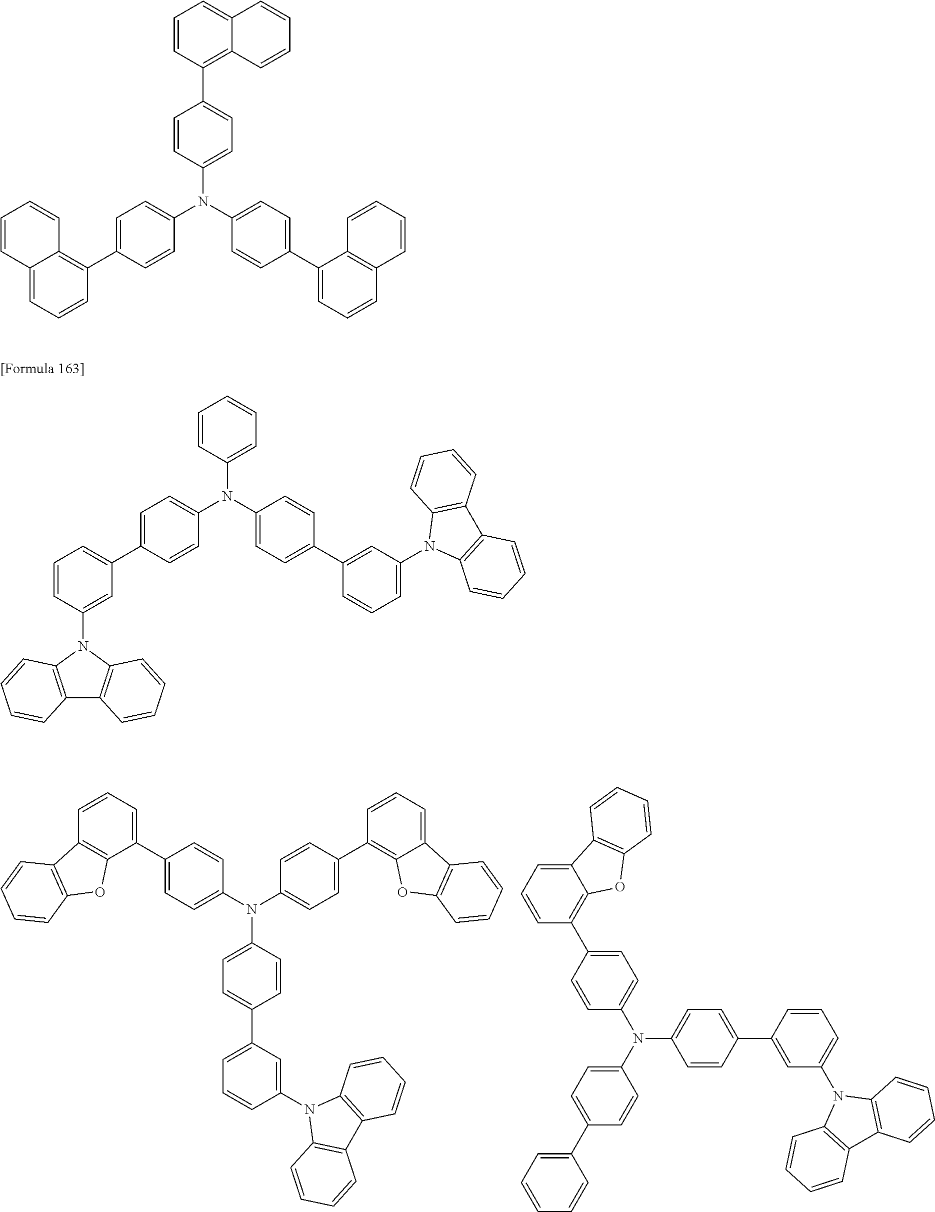

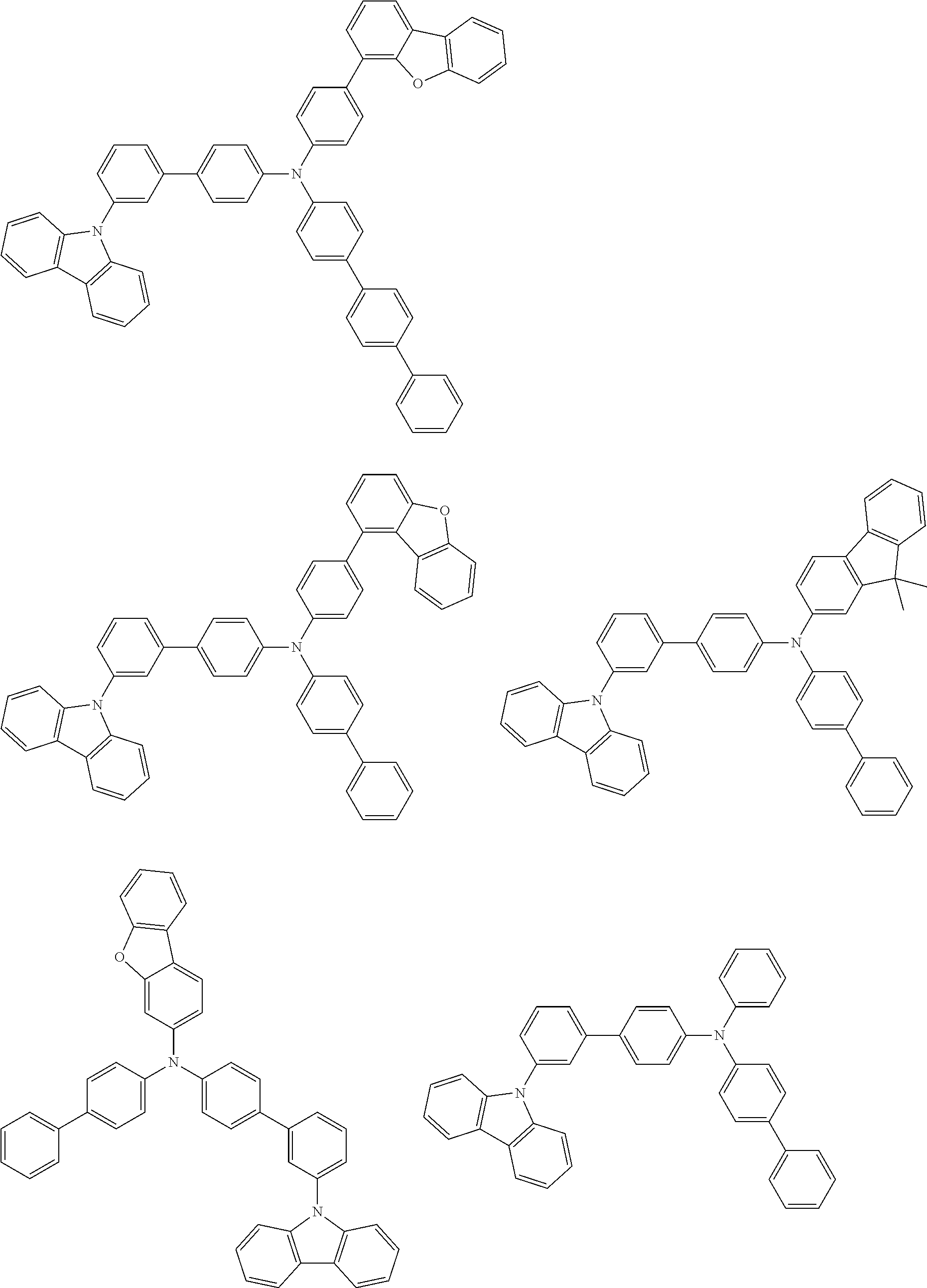

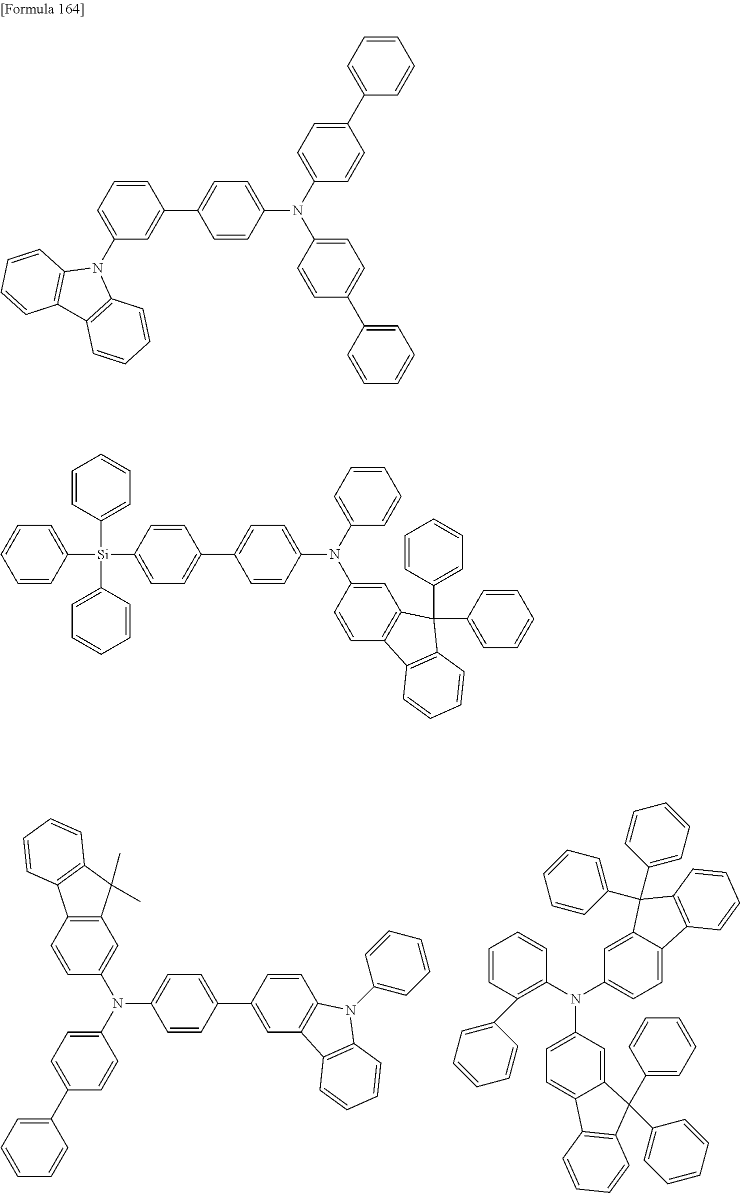 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

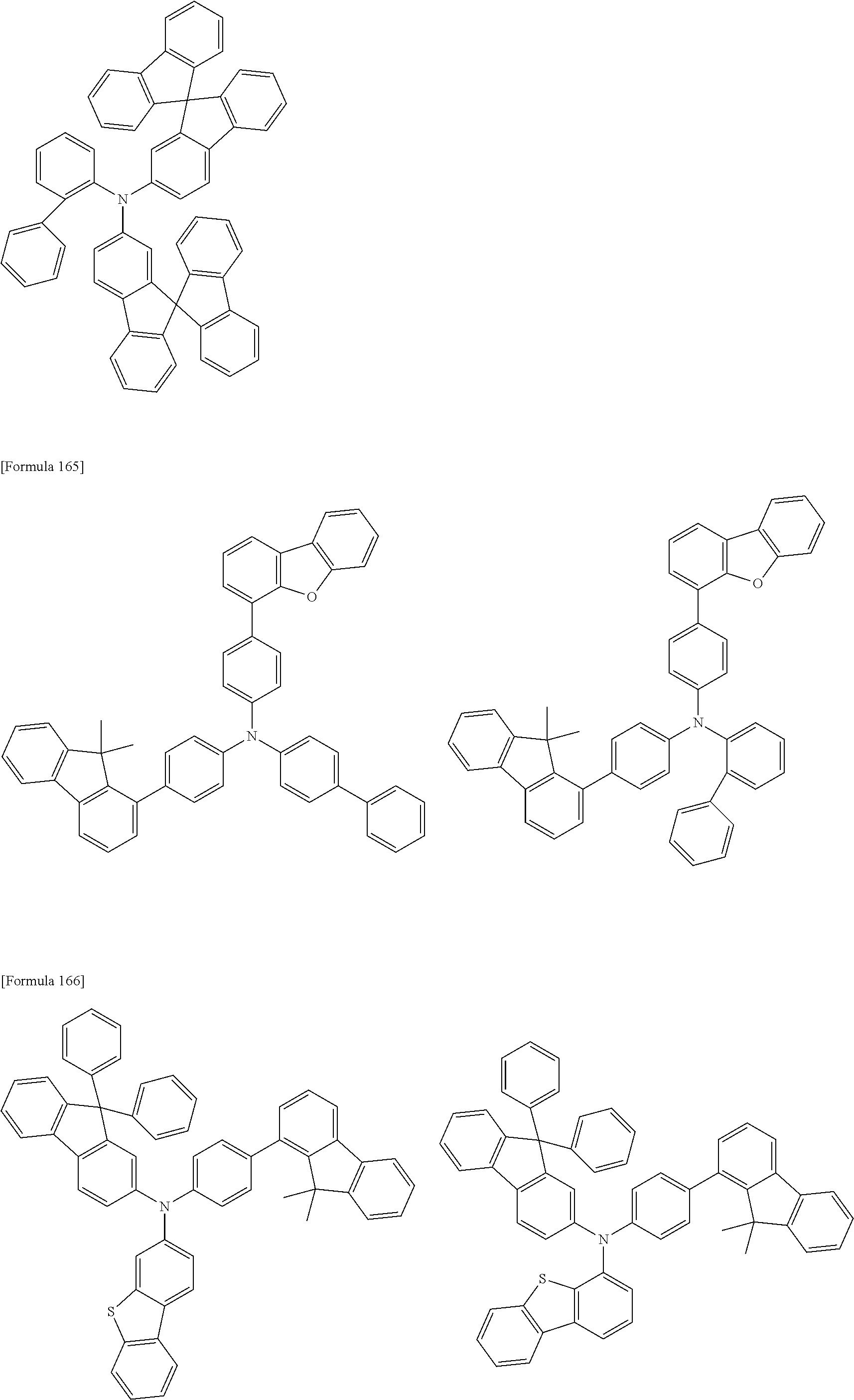 [SMILES: c1ccc(-c2ccccc2N(c2ccc3c(c2)C2(c4ccccc4-c4ccccc42)c2ccccc2-3)c2ccc3c(c2)C2(c4ccccc4-c4ccccc42)c2ccccc2-3)cc1 ; CC1(C)c2ccccc2-c2cccc(-c3ccc(N(c4ccc(-c5ccccc5)cc4)c4ccc(-c5cccc6c5oc5ccccc56)cc4)cc3)c21 ; CC1(C)c2ccccc2-c2cccc(-c3ccc(N(c4ccc(-c5cccc6c5oc5ccccc56)cc4)c4ccccc4-c4ccccc4)cc3)c21 ; CC1(C)c2ccccc2-c2cccc(-c3ccc(N(c4ccc5c(c4)C(c4ccccc4)(c4ccccc4)c4ccccc4-5)c4ccc5c(c4)sc4ccccc45)cc3)c21 ; CC1(C)c2ccccc2-c2cccc(-c3ccc(N(c4ccc5c(c4)C(c4ccccc4)(c4ccccc4)c4ccccc4-5)c4cccc5c4sc4ccccc45)cc3)c21]

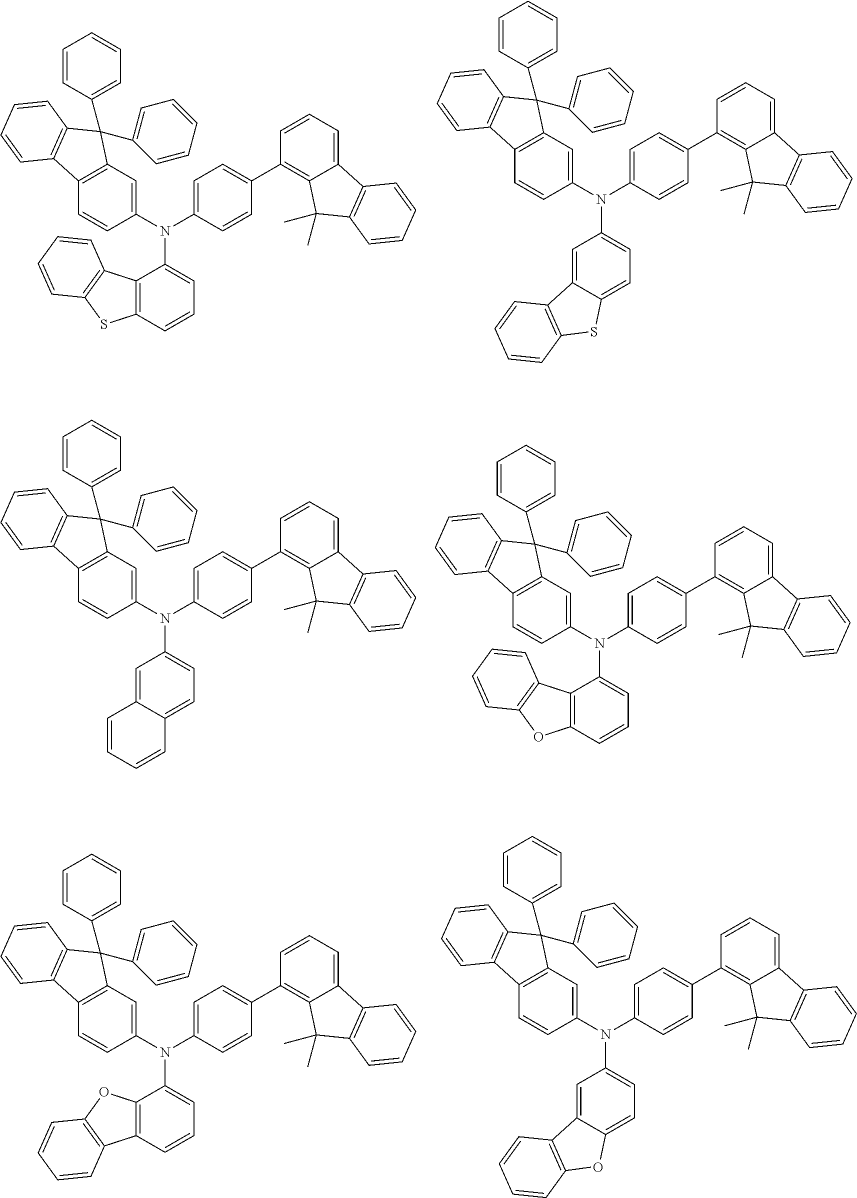

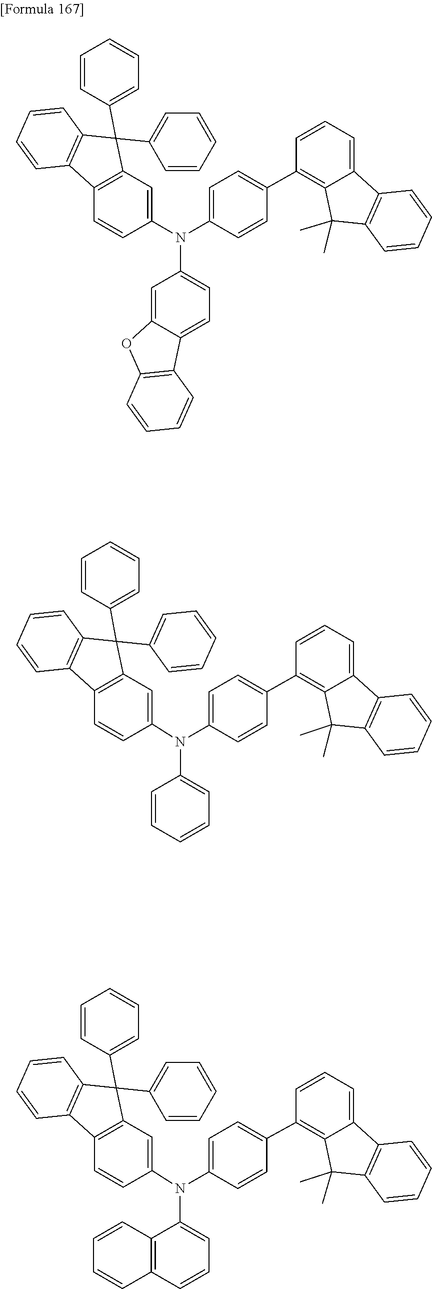 [SMILES: CC1(C)c2ccccc2-c2cccc(-c3ccc(N(c4ccc5c(c4)C(c4ccccc4)(c4ccccc4)c4ccccc4-5)c4ccc5c(c4)oc4ccccc45)cc3)c21 ; CC1(C)c2ccccc2-c2cccc(-c3ccc(N(c4ccccc4)c4ccc5c(c4)C(c4ccccc4)(c4ccccc4)c4ccccc4-5)cc3)c21 ; CC1(C)c2ccccc2-c2cccc(-c3ccc(N(c4ccc5c(c4)C(c4ccccc4)(c4ccccc4)c4ccccc4-5)c4cccc5ccccc45)cc3)c21]

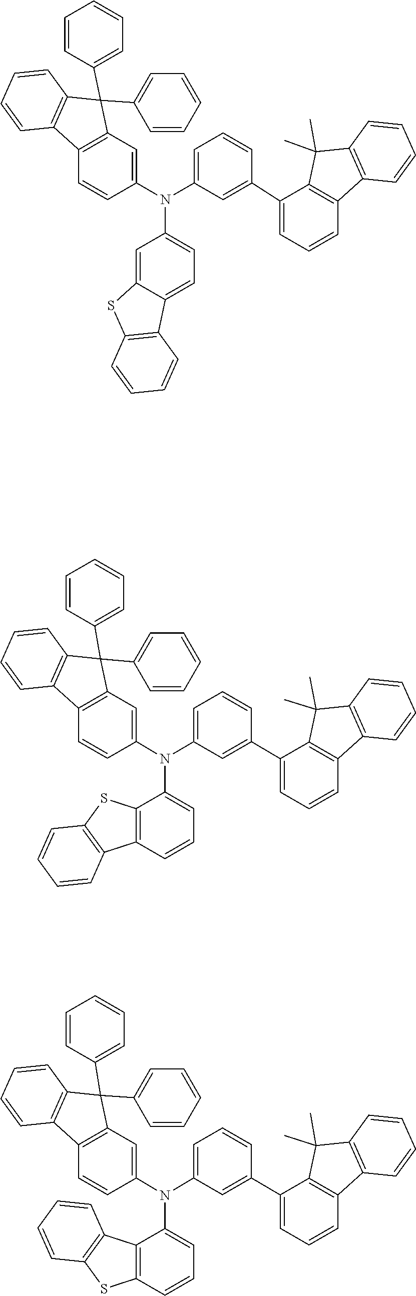 [SMILES: CC1(C)c2ccccc2-c2cccc(-c3cccc(N(c4ccc5c(c4)C(c4ccccc4)(c4ccccc4)c4ccccc4-5)c4ccc5c(c4)sc4ccccc45)c3)c21 ; CC1(C)c2ccccc2-c2cccc(-c3cccc(N(c4ccc5c(c4)C(c4ccccc4)(c4ccccc4)c4ccccc4-5)c4cccc5c4sc4ccccc45)c3)c21 ; CC1(C)c2ccccc2-c2cccc(-c3cccc(N(c4ccc5c(c4)C(c4ccccc4)(c4ccccc4)c4ccccc4-5)c4cccc5sc6ccccc6c45)c3)c21]

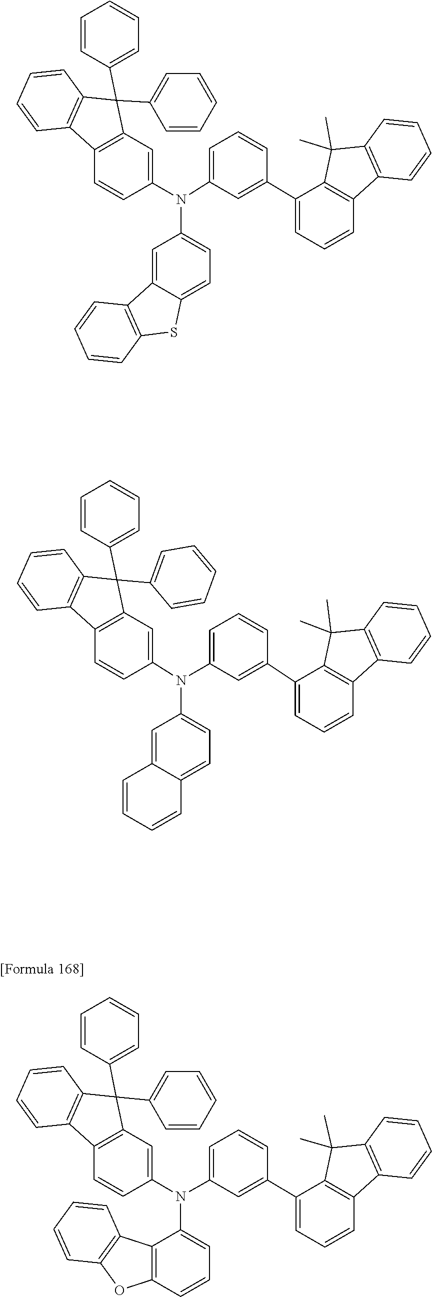 [SMILES: CC1(C)c2ccccc2-c2cccc(-c3cccc(N(c4ccc5c(c4)C(c4ccccc4)(c4ccccc4)c4ccccc4-5)c4ccc5sc6ccccc6c5c4)c3)c21 ; CC1(C)c2ccccc2-c2cccc(-c3cccc(N(c4ccc5c(c4)C(c4ccccc4)(c4ccccc4)c4ccccc4-5)c4ccc5ccccc5c4)c3)c21 ; CC1(C)c2ccccc2-c2cccc(-c3cccc(N(c4ccc5c(c4)C(c4ccccc4)(c4ccccc4)c4ccccc4-5)c4cccc5oc6ccccc6c45)c3)c21]

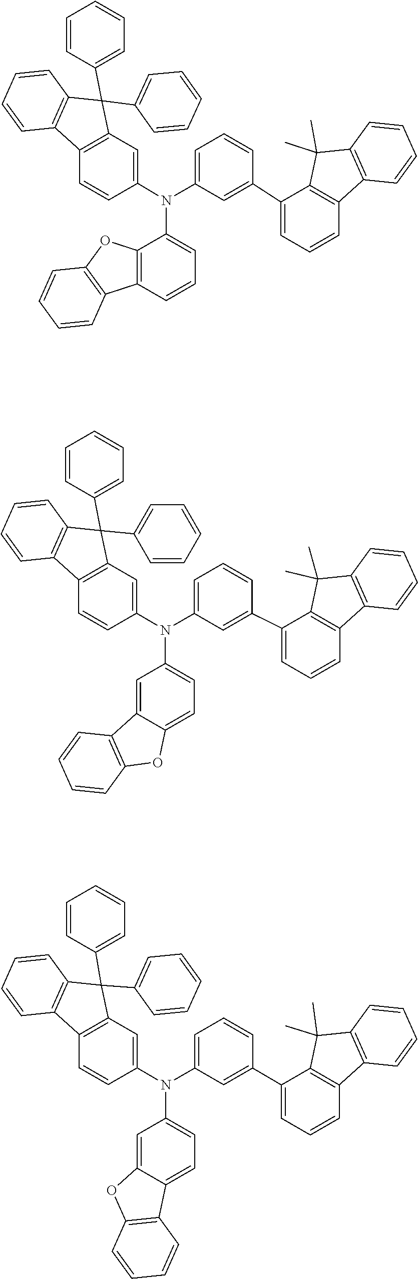 [SMILES: CC1(C)c2ccccc2-c2cccc(-c3cccc(N(c4ccc5c(c4)C(c4ccccc4)(c4ccccc4)c4ccccc4-5)c4cccc5c4oc4ccccc45)c3)c21 ; CC1(C)c2ccccc2-c2cccc(-c3cccc(N(c4ccc5c(c4)C(c4ccccc4)(c4ccccc4)c4ccccc4-5)c4ccc5oc6ccccc6c5c4)c3)c21 ; CC1(C)c2ccccc2-c2cccc(-c3cccc(N(c4ccc5c(c4)C(c4ccccc4)(c4ccccc4)c4ccccc4-5)c4ccc5c(c4)oc4ccccc45)c3)c21]

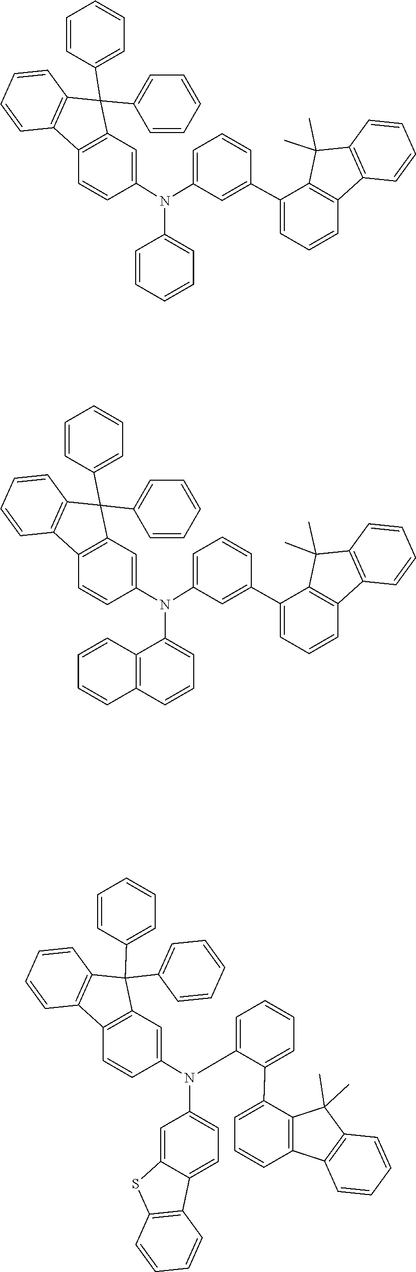 [SMILES: CC1(C)c2ccccc2-c2cccc(-c3cccc(N(c4ccccc4)c4ccc5c(c4)C(c4ccccc4)(c4ccccc4)c4ccccc4-5)c3)c21 ; CC1(C)c2ccccc2-c2cccc(-c3cccc(N(c4ccc5c(c4)C(c4ccccc4)(c4ccccc4)c4ccccc4-5)c4cccc5ccccc45)c3)c21 ; CC1(C)c2ccccc2-c2cccc(-c3ccccc3N(c3ccc4c(c3)C(c3ccccc3)(c3ccccc3)c3ccccc3-4)c3ccc4c(c3)sc3ccccc34)c21]

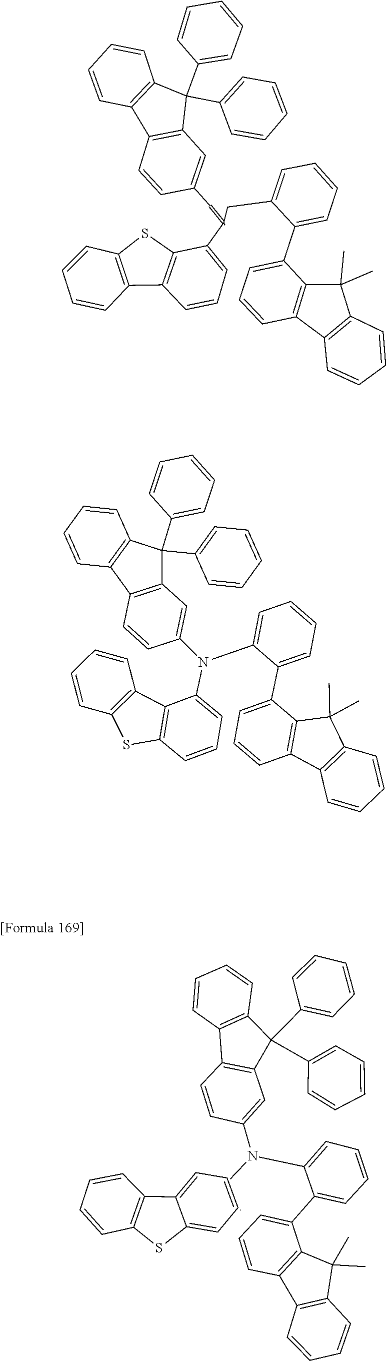

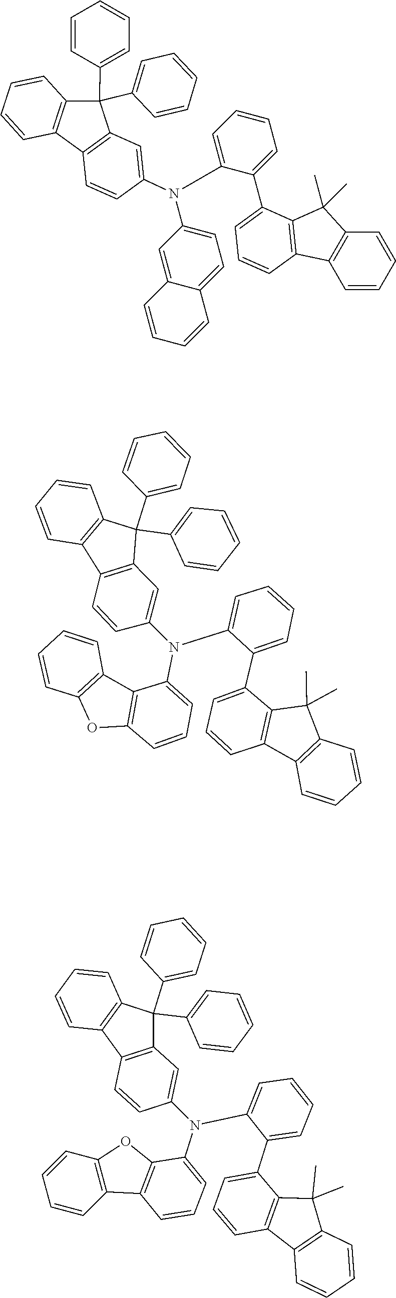 [SMILES: CC1(C)c2ccccc2-c2cccc(-c3ccccc3N(c3ccc4c(c3)C(c3ccccc3)(c3ccccc3)c3ccccc3-4)c3ccc4ccccc4c3)c21 ; CC1(C)c2ccccc2-c2cccc(-c3ccccc3N(c3ccc4c(c3)C(c3ccccc3)(c3ccccc3)c3ccccc3-4)c3cccc4oc5ccccc5c34)c21 ; CC1(C)c2ccccc2-c2cccc(-c3ccccc3N(c3ccc4c(c3)C(c3ccccc3)(c3ccccc3)c3ccccc3-4)c3cccc4c3oc3ccccc34)c21]

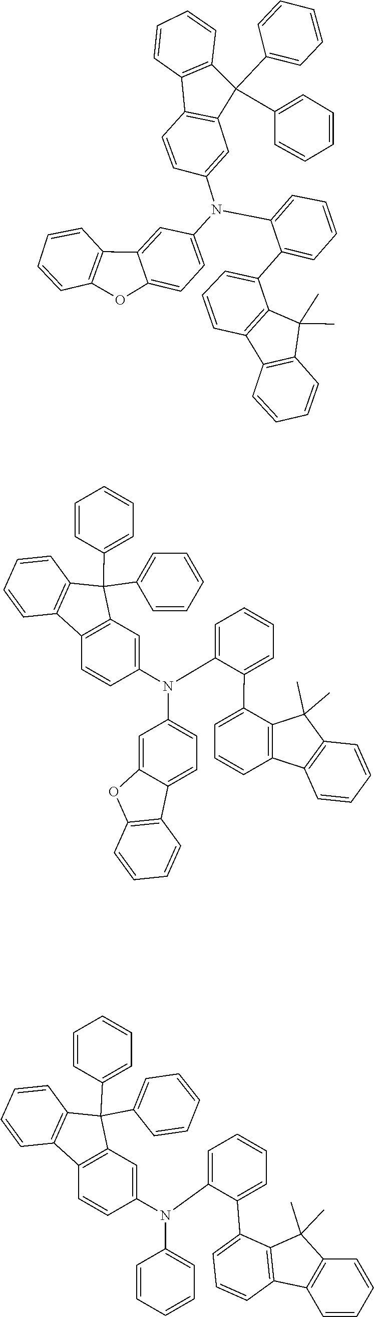 [SMILES: CC1(C)c2ccccc2-c2cccc(-c3ccccc3N(c3ccc4c(c3)C(c3ccccc3)(c3ccccc3)c3ccccc3-4)c3ccc4oc5ccccc5c4c3)c21 ; CC1(C)c2ccccc2-c2cccc(-c3ccccc3N(c3ccc4c(c3)C(c3ccccc3)(c3ccccc3)c3ccccc3-4)c3ccc4c(c3)oc3ccccc34)c21 ; CC1(C)c2ccccc2-c2cccc(-c3ccccc3N(c3ccccc3)c3ccc4c(c3)C(c3ccccc3)(c3ccccc3)c3ccccc3-4)c21]

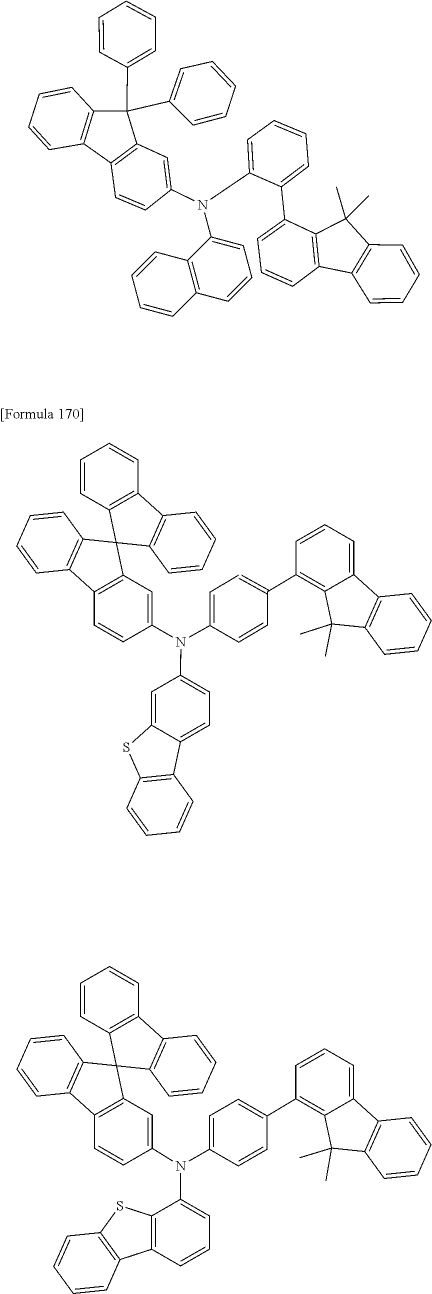 [SMILES: CC1(C)c2ccccc2-c2cccc(-c3ccccc3N(c3ccc4c(c3)C(c3ccccc3)(c3ccccc3)c3ccccc3-4)c3cccc4ccccc34)c21 ; C=C(/C=C\C(=C/C)N(c1ccc2c(c1)C1(c3ccccc3-c3ccccc31)c1ccccc1-2)c1ccc2c(c1)sc1ccccc12)c1cccc2c1C(C)(C)c1ccccc1-2 ; CC1(C)c2ccccc2-c2cccc(-c3ccc(N(c4ccc5c(c4)C4(c6ccccc6-c6ccccc64)c4ccccc4-5)c4cccc5c4sc4ccccc45)cc3)c21]

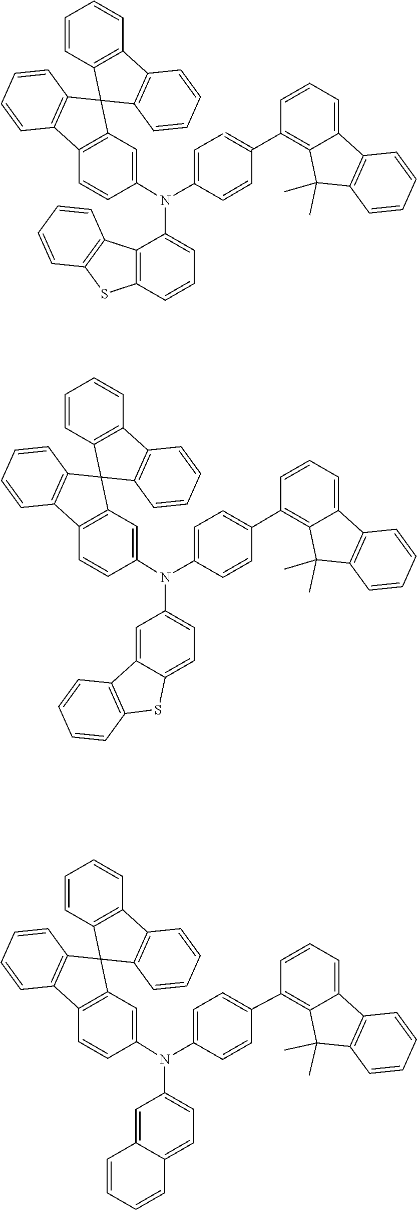 [SMILES: C=C(/C=C\C(=C/C)N(c1ccc2c(c1)C1(c3ccccc3-c3ccccc31)c1ccccc1-2)c1cccc2sc3ccccc3c12)c1cccc2c1C(C)(C)c1ccccc1-2 ; CC1(C)c2ccccc2-c2cccc(-c3ccc(N(c4ccc5c(c4)C4(c6ccccc6-c6ccccc64)c4ccccc4-5)c4ccc5sc6ccccc6c5c4)cc3)c21 ; CC1(C)c2ccccc2-c2cccc(-c3ccc(N(c4ccc5c(c4)C4(c6ccccc6-c6ccccc64)c4ccccc4-5)c4ccc5ccccc5c4)cc3)c21]

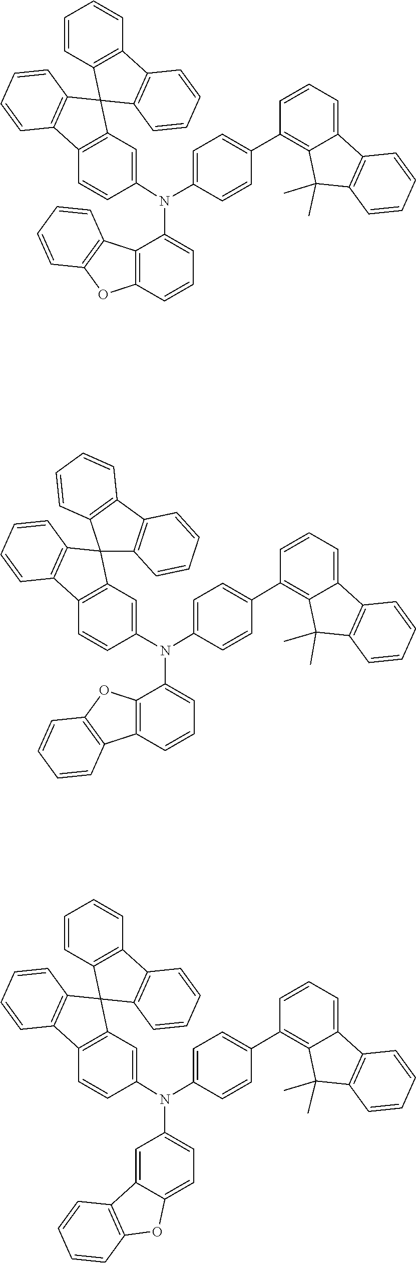 [SMILES: CC1(C)c2ccccc2-c2cccc(-c3ccc(N(c4ccc5c(c4)C4(c6ccccc6-c6ccccc64)c4ccccc4-5)c4cccc5oc6ccccc6c45)cc3)c21 ; CC1(C)c2ccccc2-c2cccc(-c3ccc(N(c4ccc5c(c4)C4(c6ccccc6-c6ccccc64)c4ccccc4-5)c4cccc5c4oc4ccccc45)cc3)c21 ; CC1(C)c2ccccc2-c2cccc(-c3ccc(N(c4ccc5c(c4)C4(c6ccccc6-c6ccccc64)c4ccccc4-5)c4ccc5oc6ccccc6c5c4)cc3)c21]

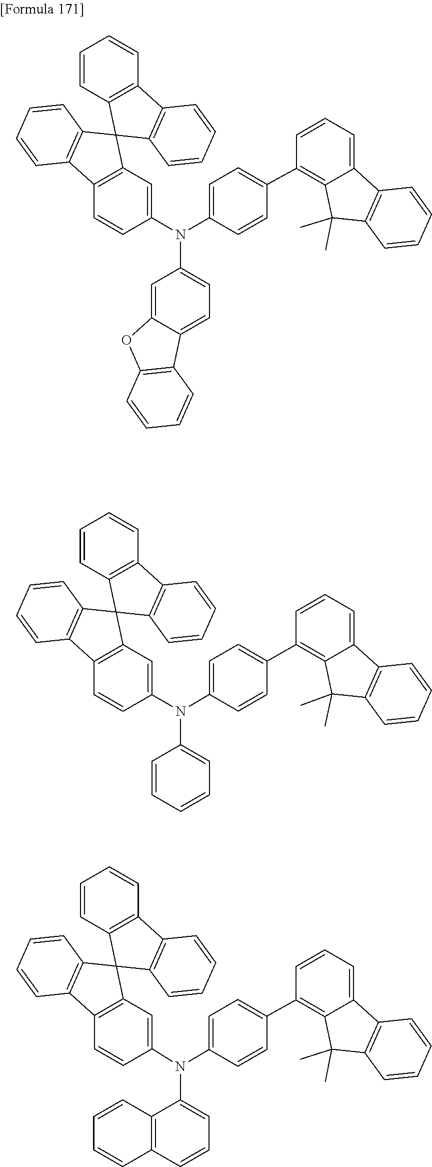 [SMILES: CC1(C)c2ccccc2-c2cccc(-c3ccc(N(c4ccc5c(c4)C4(c6ccccc6-c6ccccc64)c4ccccc4-5)c4ccc5c(c4)oc4ccccc45)cc3)c21 ; CC1(C)c2ccccc2-c2cccc(-c3ccc(N(c4ccccc4)c4ccc5c(c4)C4(c6ccccc6-c6ccccc64)c4ccccc4-5)cc3)c21 ; CC1(C)c2ccccc2-c2cccc(-c3ccc(N(c4ccc5c(c4)C4(c6ccccc6-c6ccccc64)c4ccccc4-5)c4cccc5ccccc45)cc3)c21]

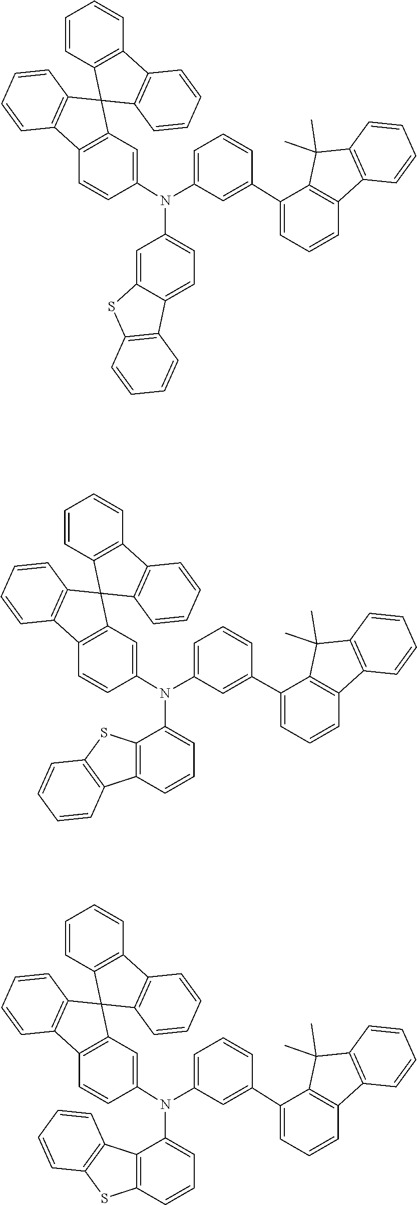 [SMILES: CC1(C)c2ccccc2-c2cccc(-c3cccc(N(c4ccc5c(c4)C4(c6ccccc6-c6ccccc64)c4ccccc4-5)c4ccc5c(c4)sc4ccccc45)c3)c21 ; CC1(C)c2ccccc2-c2cccc(-c3cccc(N(c4ccc5c(c4)C4(c6ccccc6-c6ccccc64)c4ccccc4-5)c4cccc5c4sc4ccccc45)c3)c21 ; CC1(C)c2ccccc2-c2cccc(-c3cccc(N(c4ccc5c(c4)C4(c6ccccc6-c6ccccc64)c4ccccc4-5)c4cccc5sc6ccccc6c45)c3)c21]

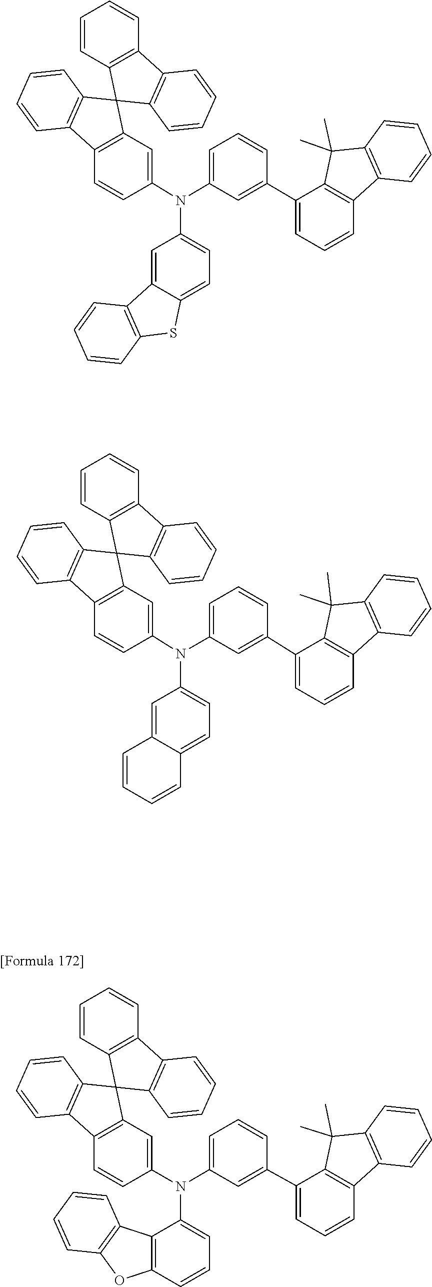 [SMILES: CC1(C)c2ccccc2-c2cccc(-c3cccc(N(c4ccc5c(c4)C4(c6ccccc6-c6ccccc64)c4ccccc4-5)c4ccc5sc6ccccc6c5c4)c3)c21 ; CC1(C)c2ccccc2-c2cccc(-c3cccc(N(c4ccc5c(c4)C4(c6ccccc6-c6ccccc64)c4ccccc4-5)c4ccc5ccccc5c4)c3)c21 ; CC1(C)c2ccccc2-c2cccc(-c3cccc(N(c4ccc5c(c4)C4(c6ccccc6-c6ccccc64)c4ccccc4-5)c4cccc5oc6ccccc6c45)c3)c21]

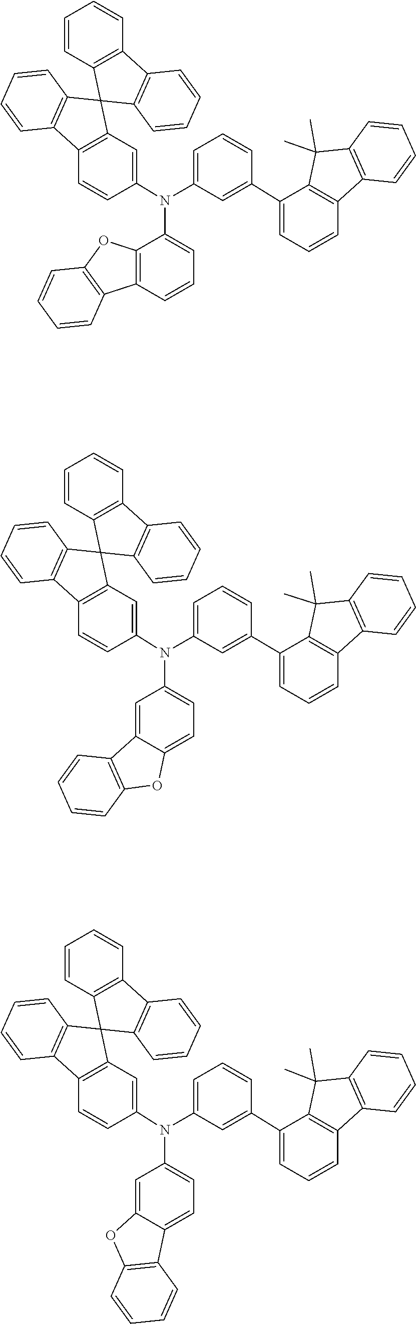 [SMILES: CC1(C)c2ccccc2-c2cccc(-c3cccc(N(c4ccc5c(c4)C4(c6ccccc6-c6ccccc64)c4ccccc4-5)c4cccc5c4oc4ccccc45)c3)c21 ; CC1(C)c2ccccc2-c2cccc(-c3cccc(N(c4ccc5c(c4)C4(c6ccccc6-c6ccccc64)c4ccccc4-5)c4ccc5oc6ccccc6c5c4)c3)c21 ; CC1(C)c2ccccc2-c2cccc(-c3cccc(N(c4ccc5c(c4)C4(c6ccccc6-c6ccccc64)c4ccccc4-5)c4ccc5c(c4)oc4ccccc45)c3)c21]

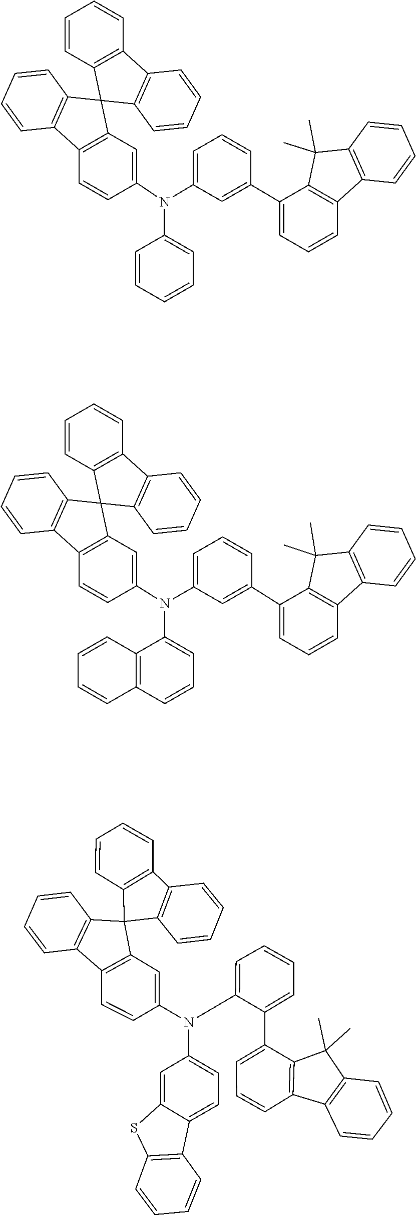 [SMILES: CC1(C)c2ccccc2-c2cccc(-c3cccc(N(c4ccccc4)c4ccc5c(c4)C4(c6ccccc6-c6ccccc64)c4ccccc4-5)c3)c21 ; CC1(C)c2ccccc2-c2cccc(-c3cccc(N(c4ccc5c(c4)C4(c6ccccc6-c6ccccc64)c4ccccc4-5)c4cccc5ccccc45)c3)c21 ; CC1(C)c2ccccc2-c2cccc(-c3ccccc3N(c3ccc4c(c3)C3(c5ccccc5-c5ccccc53)c3ccccc3-4)c3ccc4c(c3)sc3ccccc34)c21]

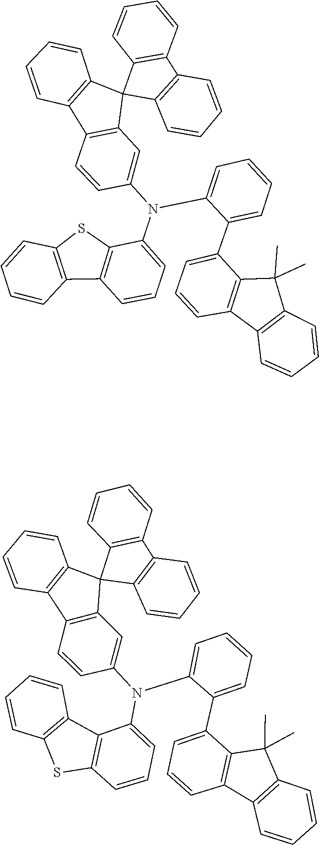 [SMILES: CC1(C)c2ccccc2-c2cccc(-c3ccccc3N(c3ccc4c(c3)C3(c5ccccc5-c5ccccc53)c3ccccc3-4)c3cccc4c3sc3ccccc34)c21 ; CC1(C)c2ccccc2-c2cccc(-c3ccccc3N(c3ccc4c(c3)C3(c5ccccc5-c5ccccc53)c3ccccc3-4)c3cccc4sc5ccccc5c34)c21]

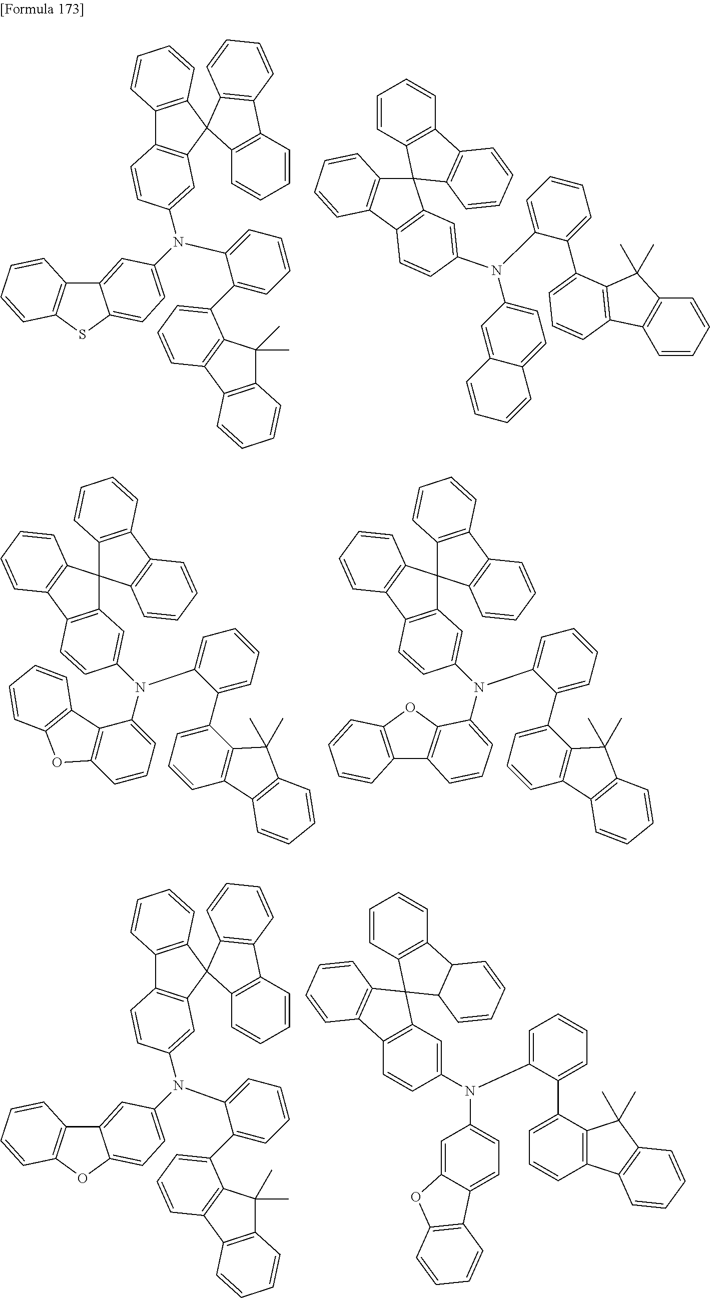

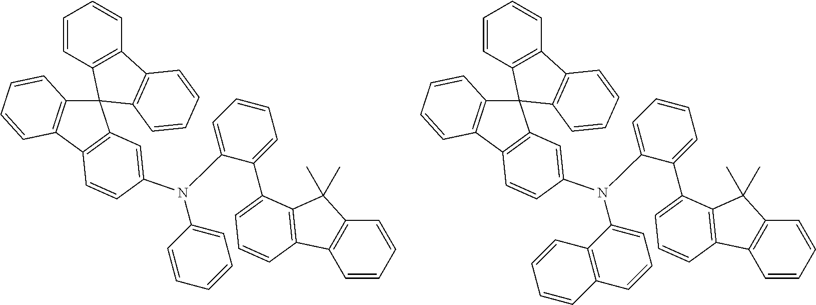 [SMILES: CC1(C)c2ccccc2-c2cccc(-c3ccccc3N(c3ccccc3)c3ccc4c(c3)C3(c5ccccc5-c5ccccc53)c3ccccc3-4)c21 ; CC1(C)c2ccccc2-c2cccc(-c3ccccc3N(c3ccc4c(c3)C3(c5ccccc5-c5ccccc53)c3ccccc3-4)c3cccc4ccccc34)c21]

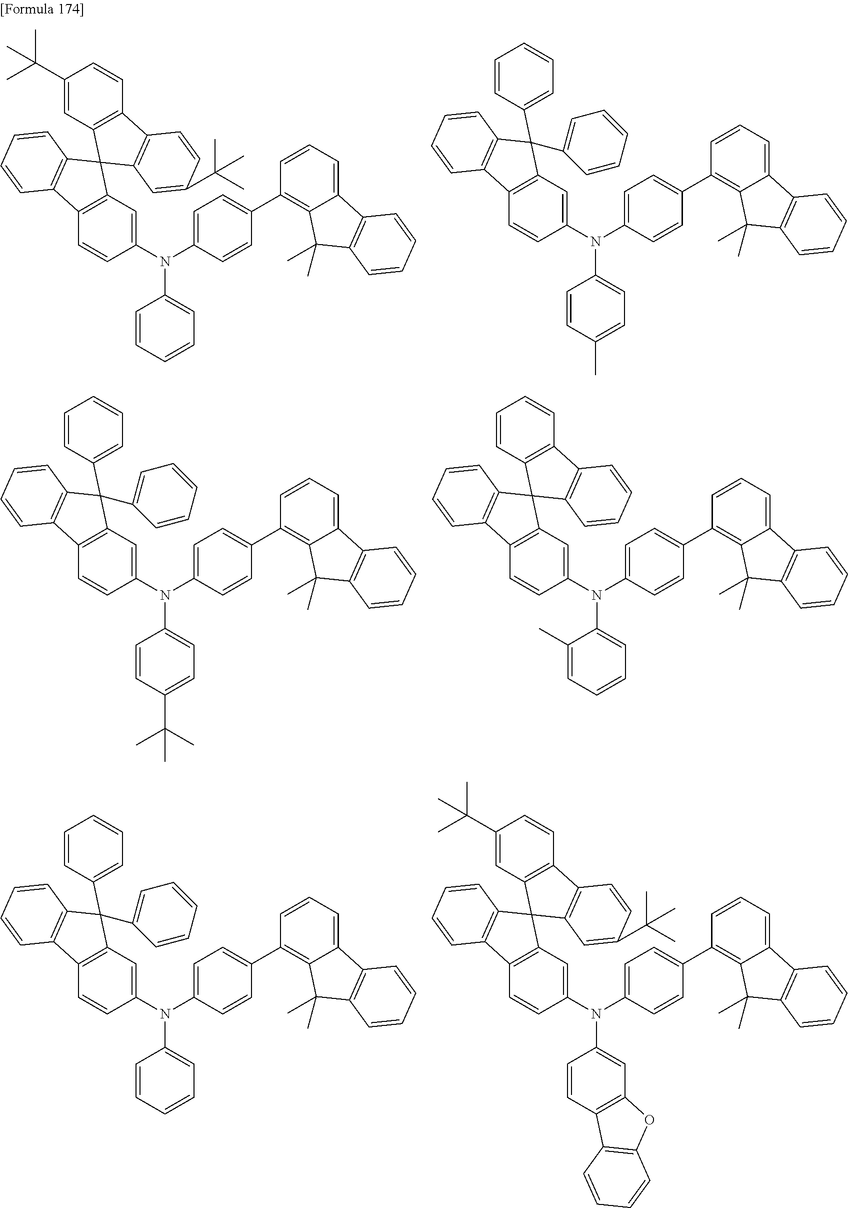

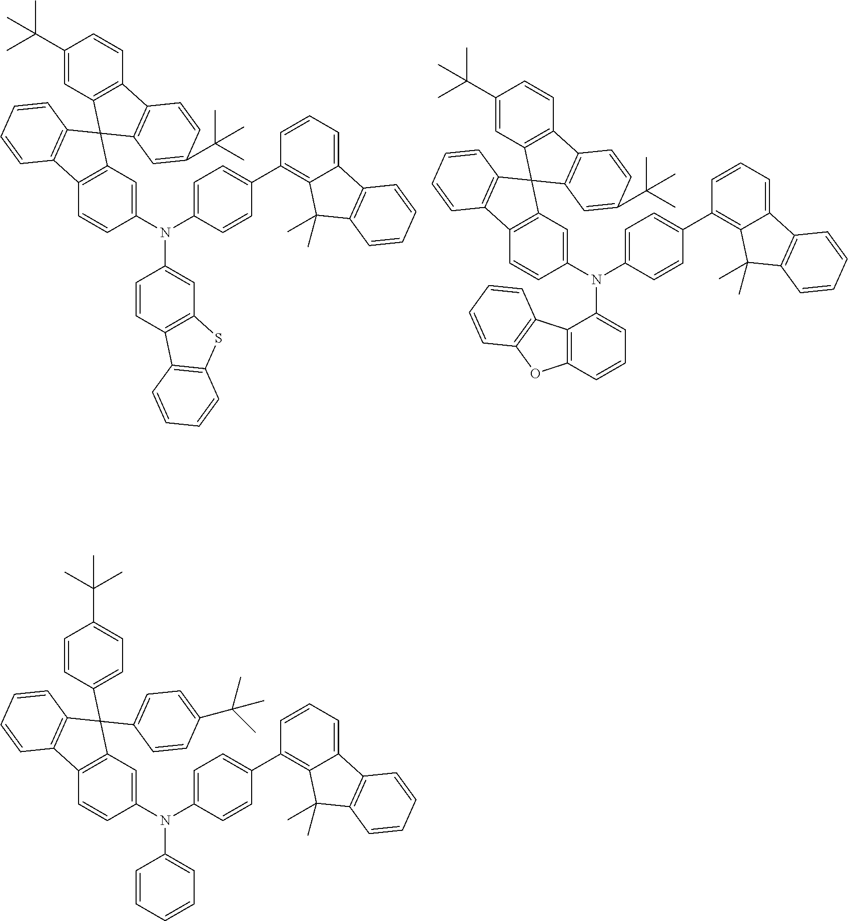

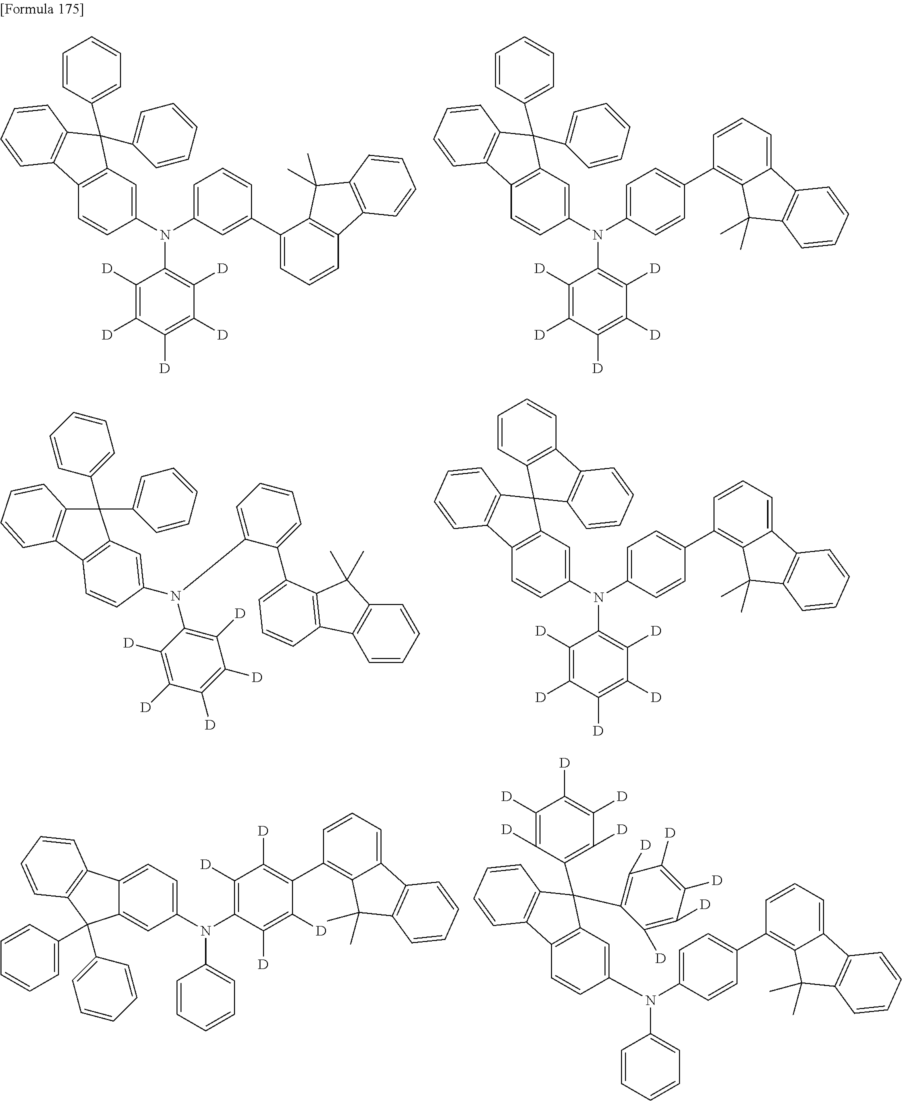

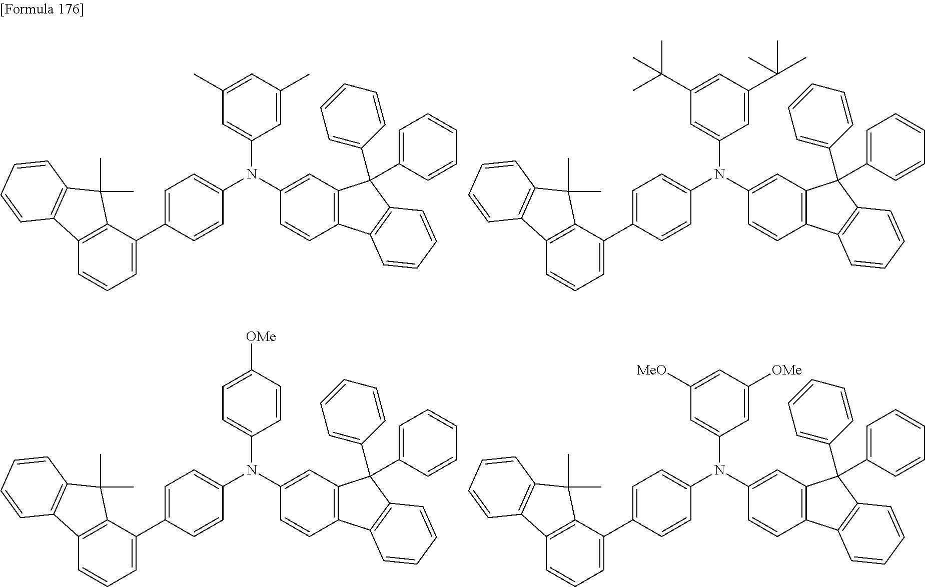

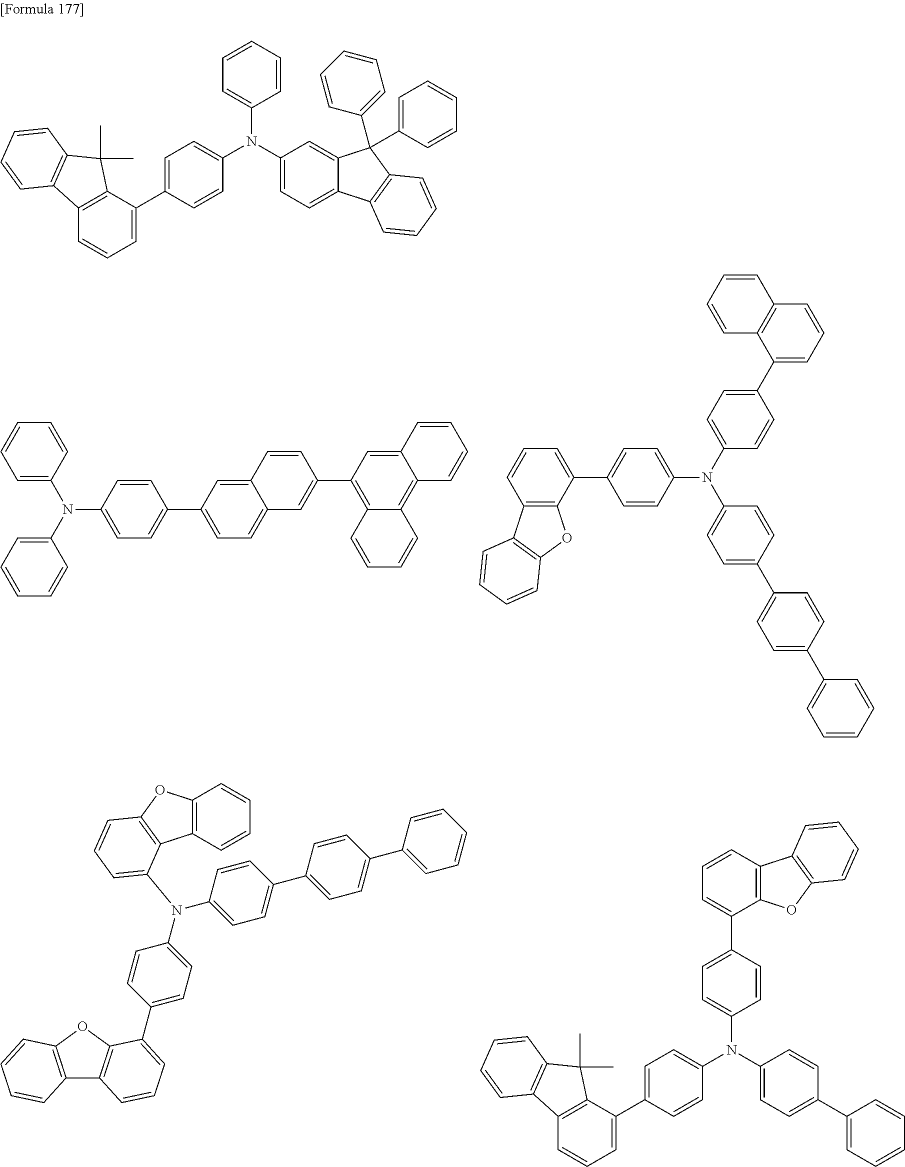 [SMILES: CC1(C)c2ccccc2-c2cccc(-c3ccc(N(c4ccccc4)c4ccc5c(c4)C(c4ccccc4)(c4ccccc4)c4ccccc4-5)cc3)c21 ; c1ccc(N(c2ccccc2)c2ccc(-c3ccc4cc(-c5cc6ccccc6c6ccccc56)ccc4c3)cc2)cc1 ; c1ccc(-c2ccc(-c3ccc(N(c4ccc(-c5cccc6ccccc56)cc4)c4ccc(-c5cccc6c5oc5ccccc56)cc4)cc3)cc2)cc1 ; c1ccc(-c2ccc(-c3ccc(N(c4ccc(-c5cccc6c5oc5ccccc56)cc4)c4cccc5oc6ccccc6c45)cc3)cc2)cc1 ; CC1(C)c2ccccc2-c2cccc(-c3ccc(N(c4ccc(-c5ccccc5)cc4)c4ccc(-c5cccc6c5oc5ccccc56)cc4)cc3)c21]

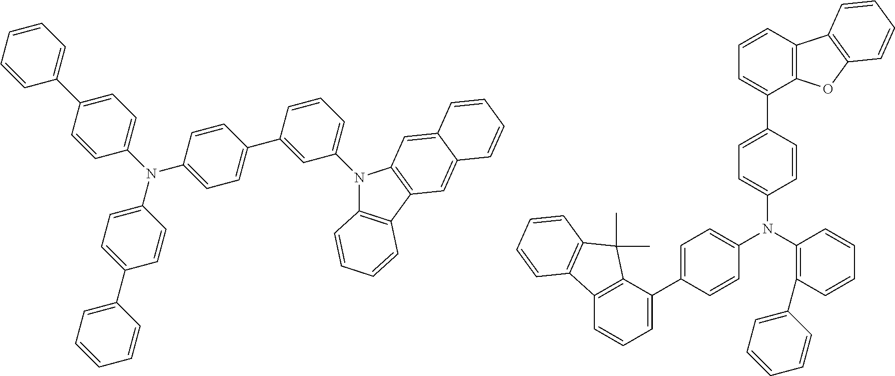 [SMILES: c1ccc(-c2ccc(N(c3ccc(-c4ccccc4)cc3)c3ccc(-c4cccc(-n5c6ccccc6c6cc7ccccc7cc65)c4)cc3)cc2)cc1 ; CC1(C)c2ccccc2-c2cccc(-c3ccc(N(c4ccc(-c5cccc6c5oc5ccccc56)cc4)c4ccccc4-c4ccccc4)cc3)c21]

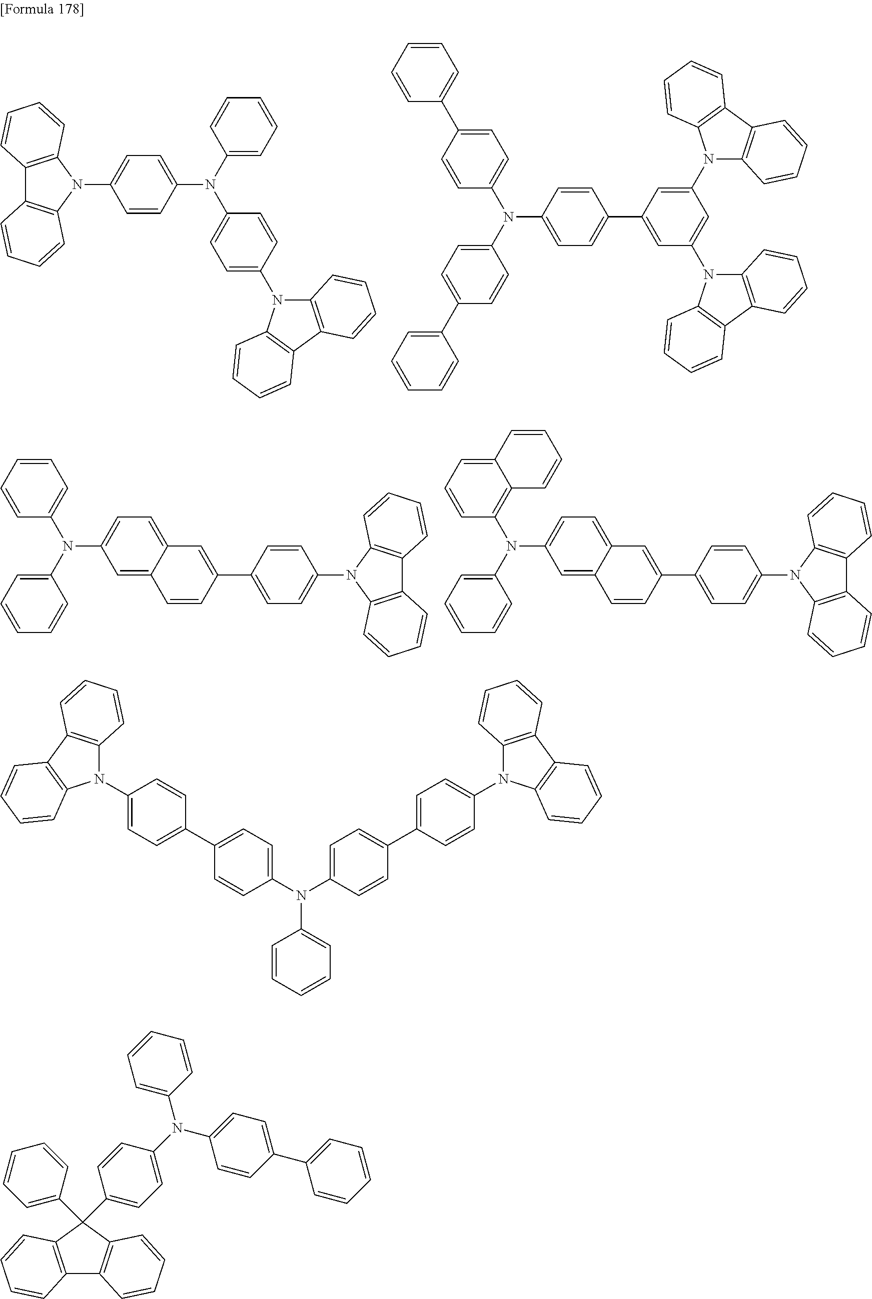

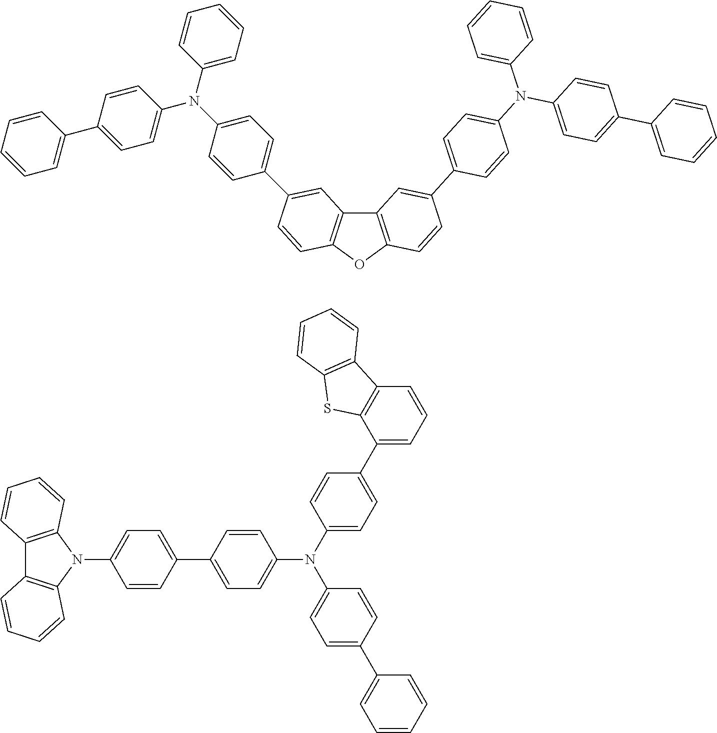 [SMILES: c1ccc(-c2ccc(N(c3ccccc3)c3ccc(-c4ccc5oc6ccc(-c7ccc(N(c8ccccc8)c8ccc(-c9ccccc9)cc8)cc7)cc6c5c4)cc3)cc2)cc1 ; c1ccc(-c2ccc(N(c3ccc(-c4ccc(-n5c6ccccc6c6ccccc65)cc4)cc3)c3ccc(-c4cccc5c4sc4ccccc45)cc3)cc2)cc1]

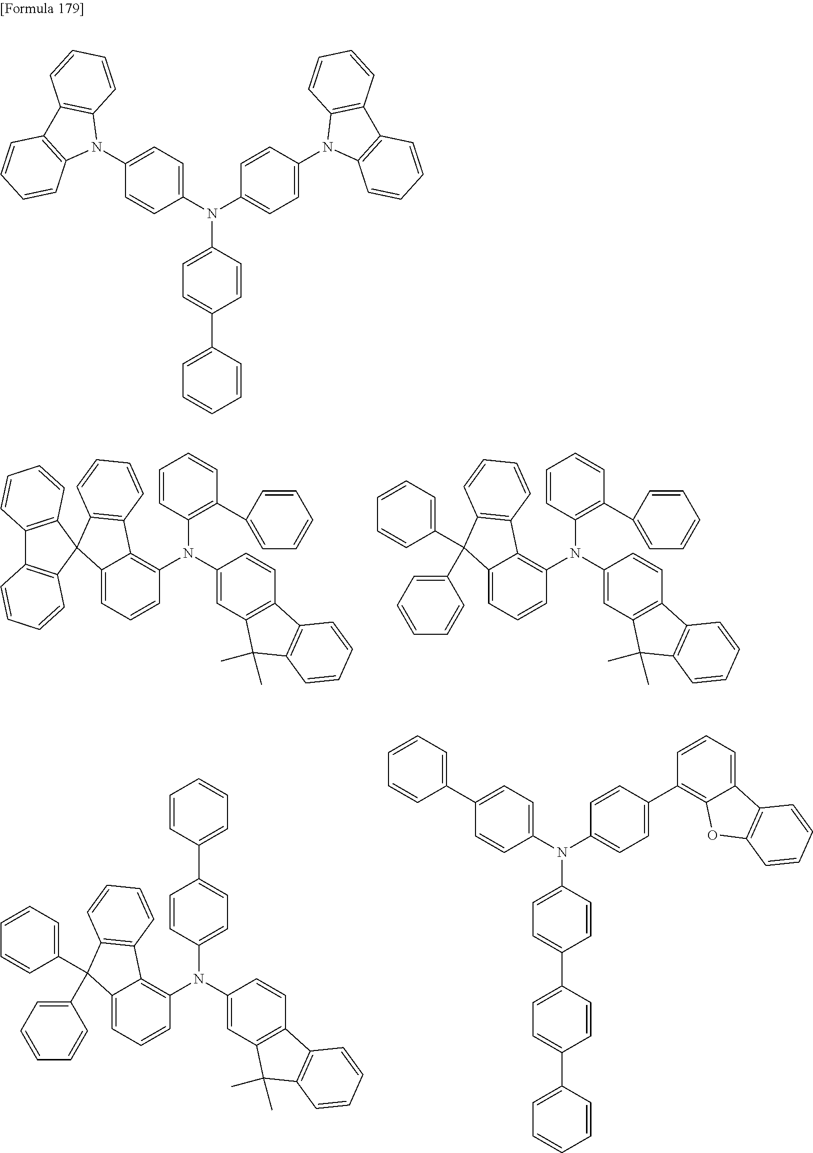

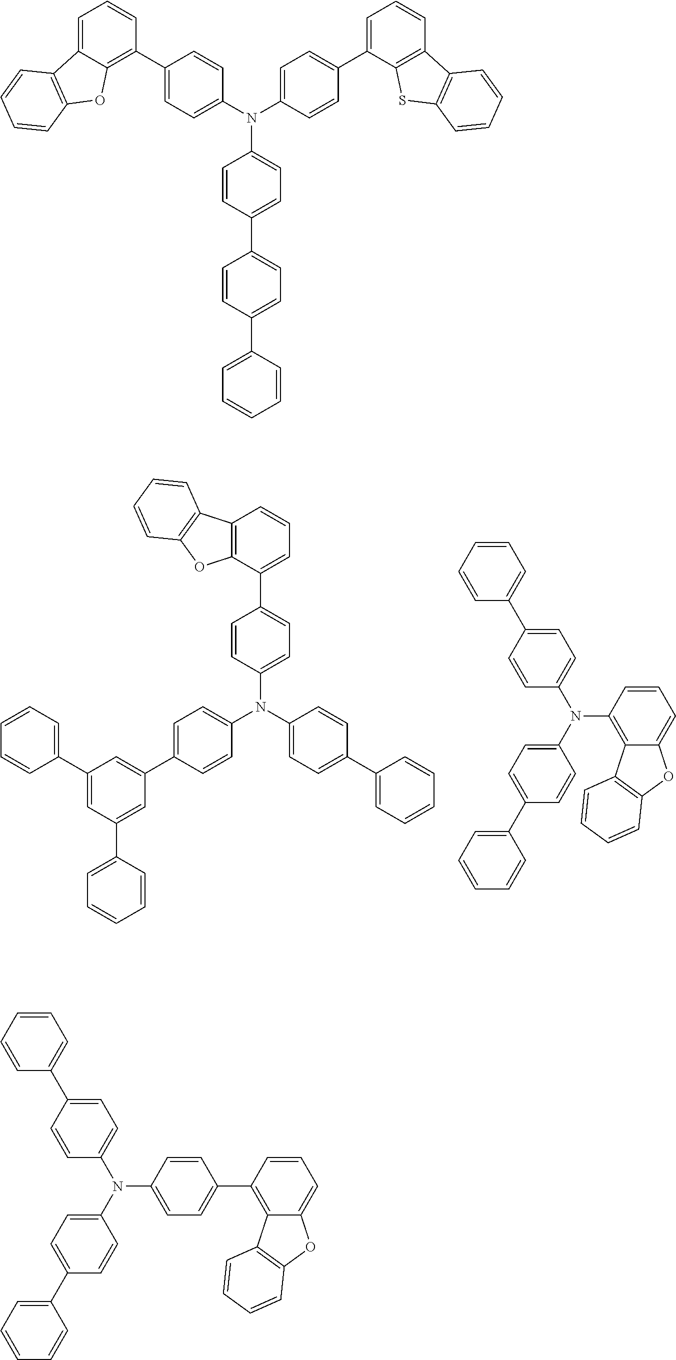 [SMILES: c1ccc(-c2ccc(-c3ccc(N(c4ccc(-c5cccc6c5oc5ccccc56)cc4)c4ccc(-c5cccc6c5sc5ccccc56)cc4)cc3)cc2)cc1 ; c1ccc(-c2ccc(N(c3ccc(-c4cc(-c5ccccc5)cc(-c5ccccc5)c4)cc3)c3ccc(-c4cccc5c4oc4ccccc45)cc3)cc2)cc1 ; c1ccc(-c2ccc(N(c3ccc(-c4ccccc4)cc3)c3cccc4oc5ccccc5c34)cc2)cc1 ; c1ccc(-c2ccc(N(c3ccc(-c4ccccc4)cc3)c3ccc(-c4cccc5oc6ccccc6c45)cc3)cc2)cc1]

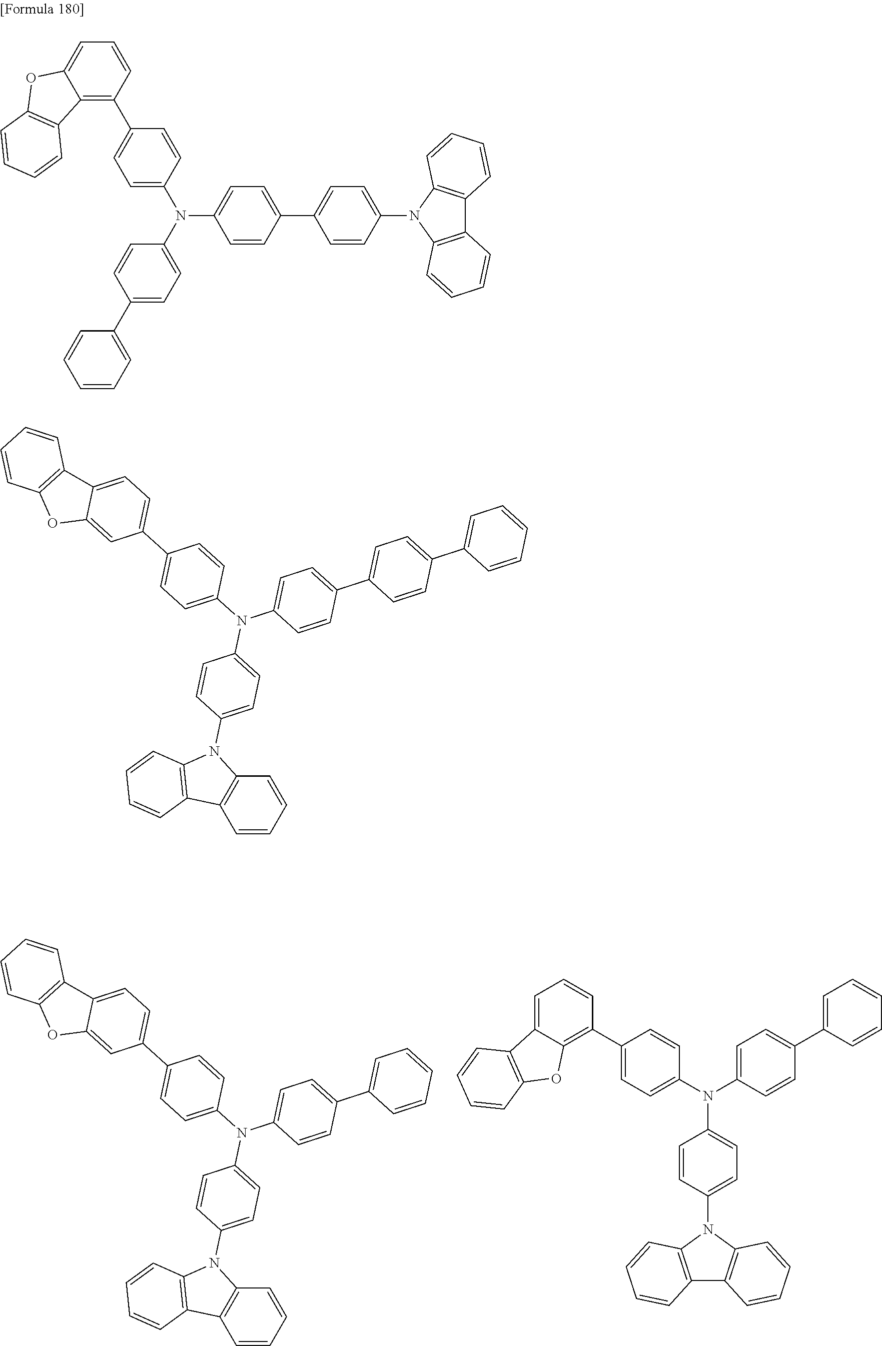

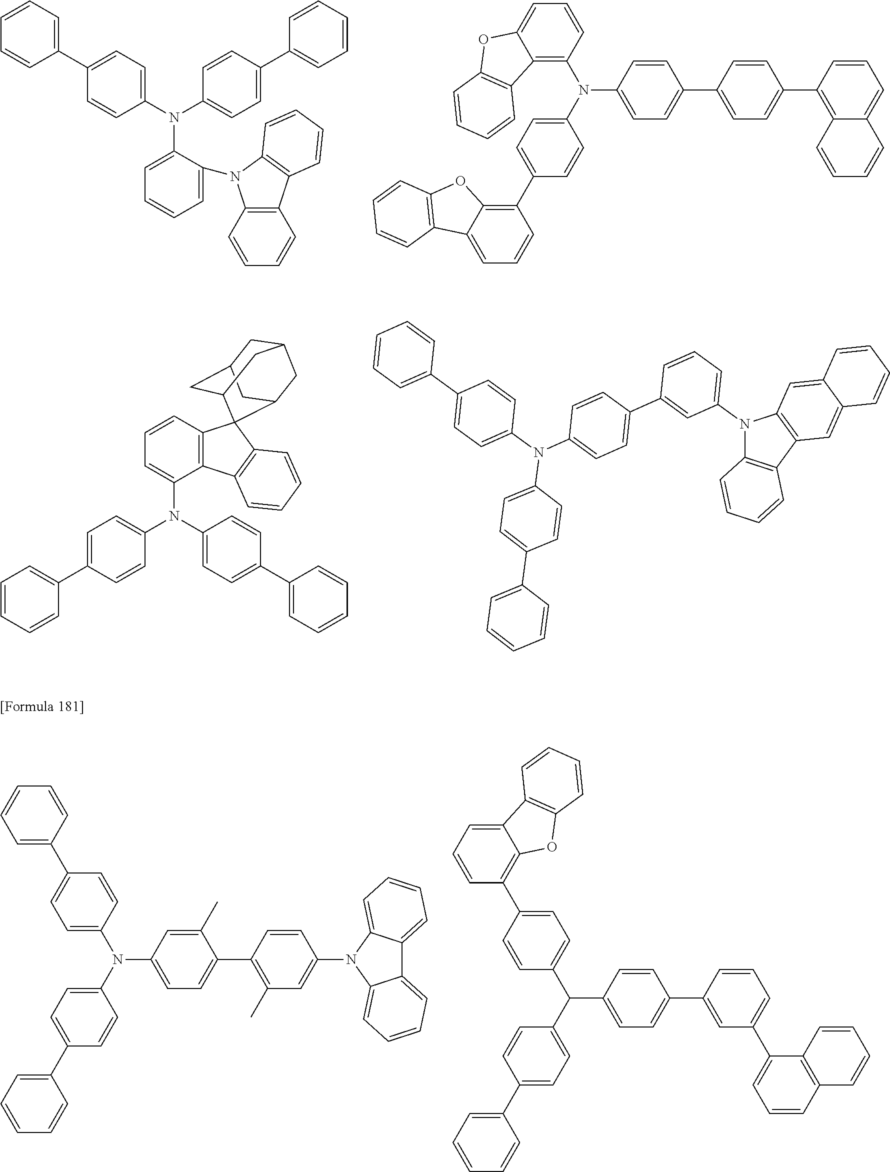

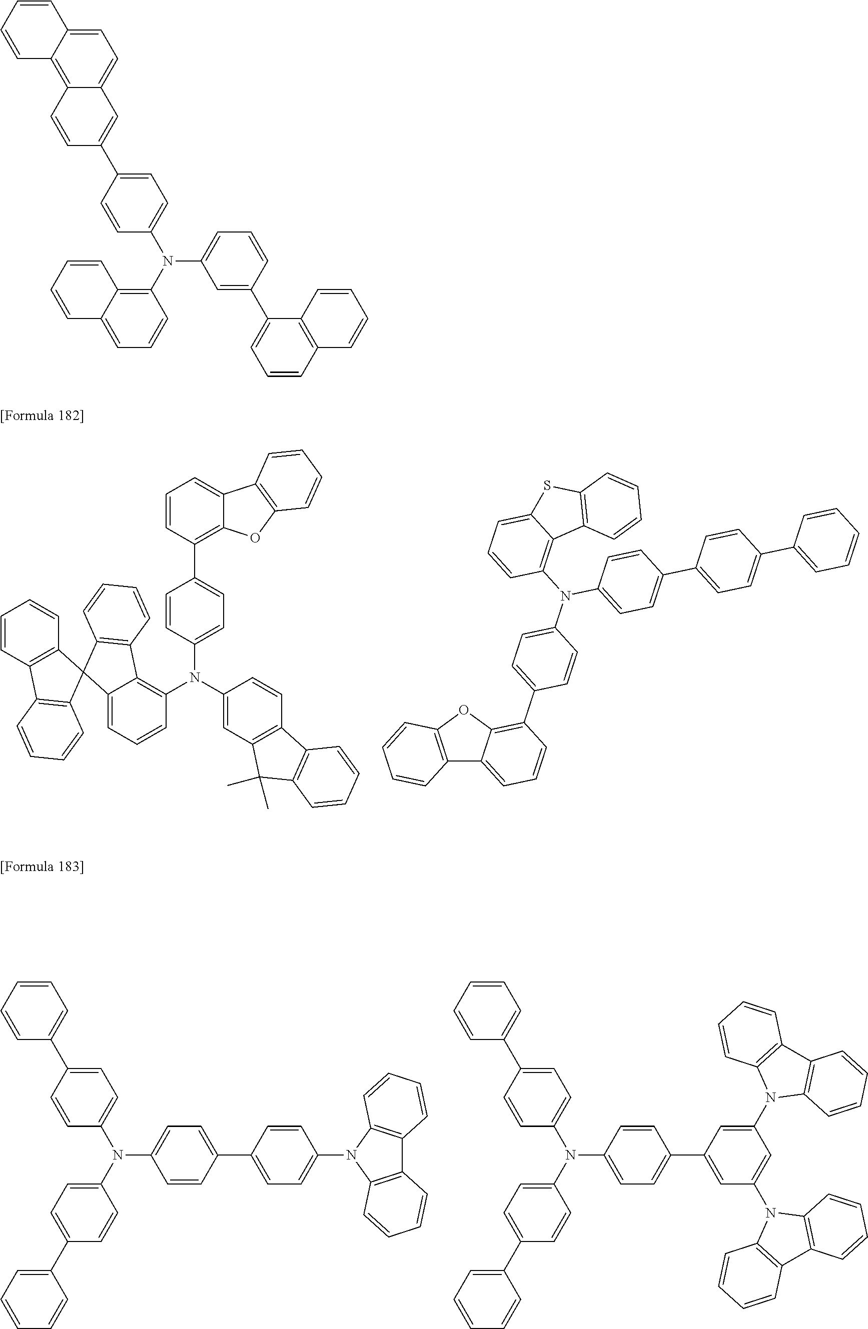

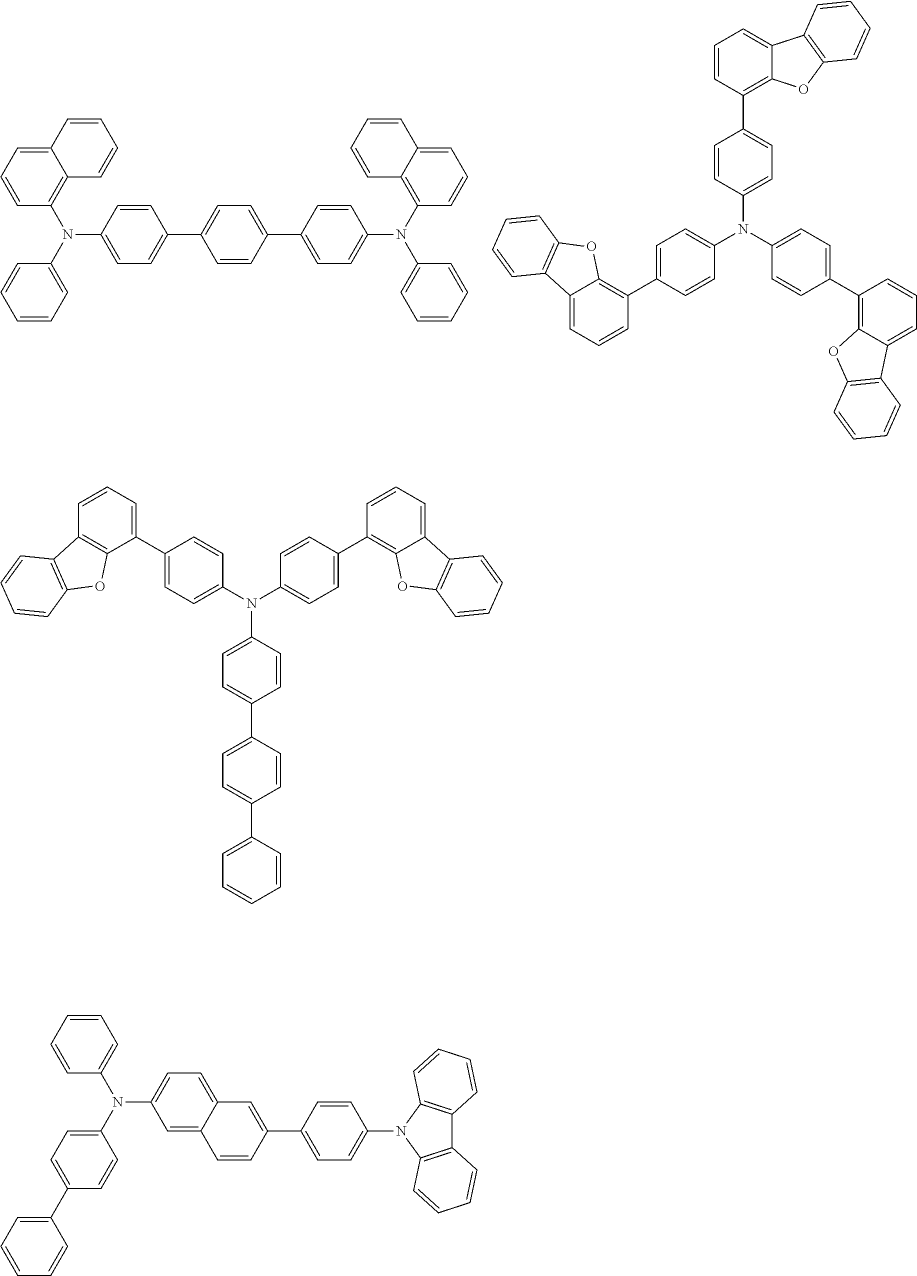

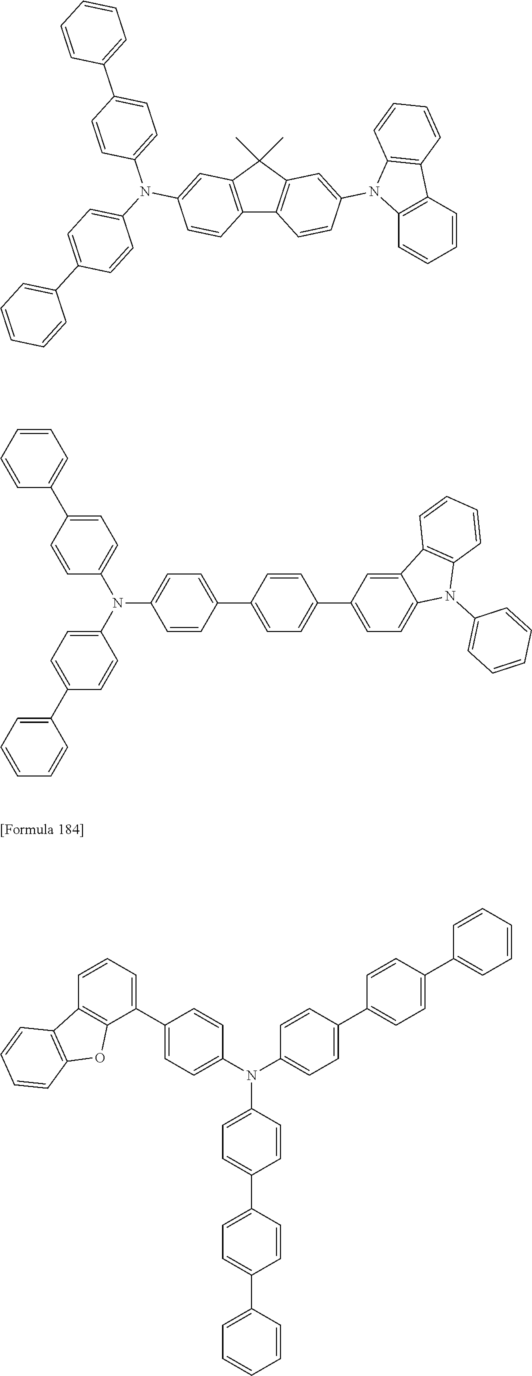 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; c1ccc(-c2ccc(-c3ccc(N(c4ccc(-c5ccc(-c6ccccc6)cc5)cc4)c4ccc(-c5cccc6c5oc5ccccc56)cc4)cc3)cc2)cc1]

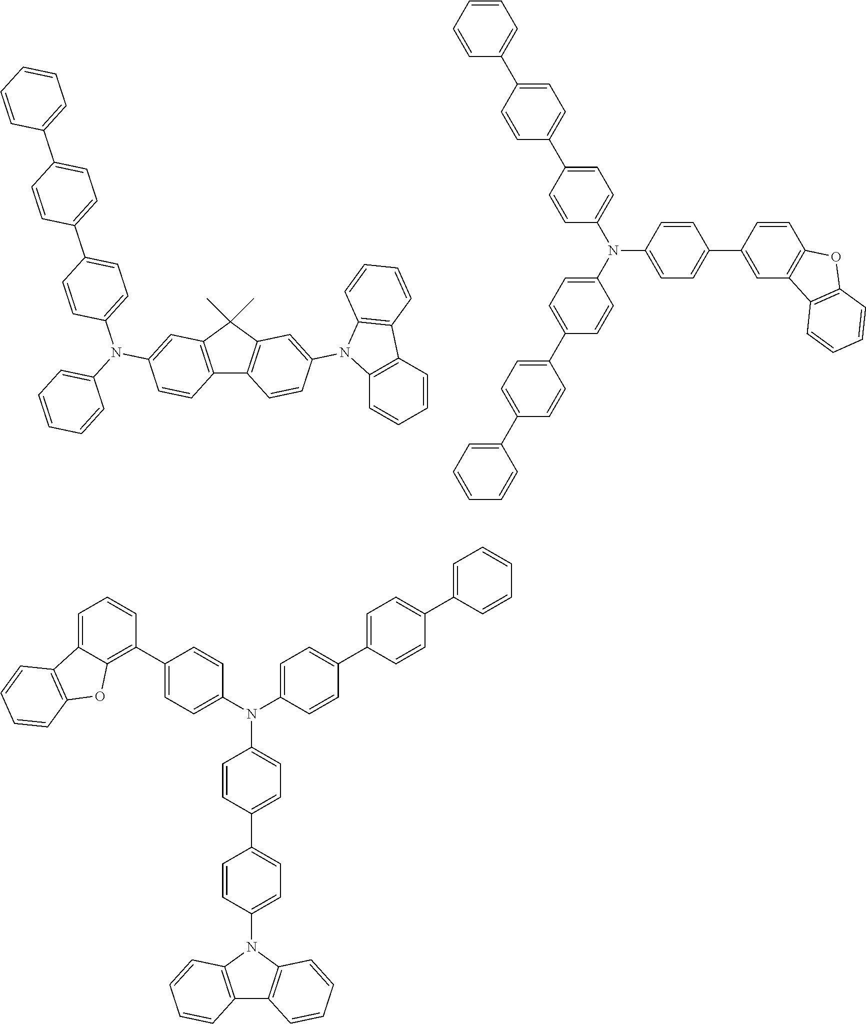 [SMILES: CC1(C)c2cc(N(c3ccccc3)c3ccc(-c4ccc(-c5ccccc5)cc4)cc3)ccc2-c2ccc(-n3c4ccccc4c4ccccc43)cc21 ; c1ccc(-c2ccc(-c3ccc(N(c4ccc(-c5ccc(-c6ccccc6)cc5)cc4)c4ccc(-c5ccc6oc7ccccc7c6c5)cc4)cc3)cc2)cc1 ; c1ccc(-c2ccc(-c3ccc(N(c4ccc(-c5ccc(-n6c7ccccc7c7ccccc76)cc5)cc4)c4ccc(-c5cccc6c5oc5ccccc56)cc4)cc3)cc2)cc1]

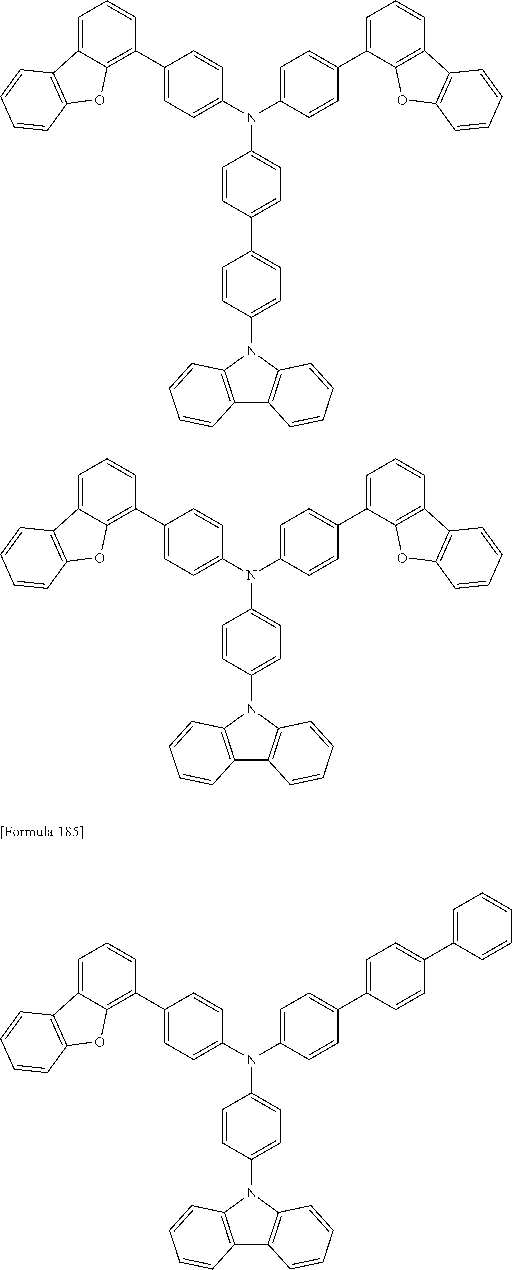 [SMILES: c1ccc2c(c1)oc1c(-c3ccc(N(c4ccc(-c5ccc(-n6c7ccccc7c7ccccc76)cc5)cc4)c4ccc(-c5cccc6c5oc5ccccc56)cc4)cc3)cccc12 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

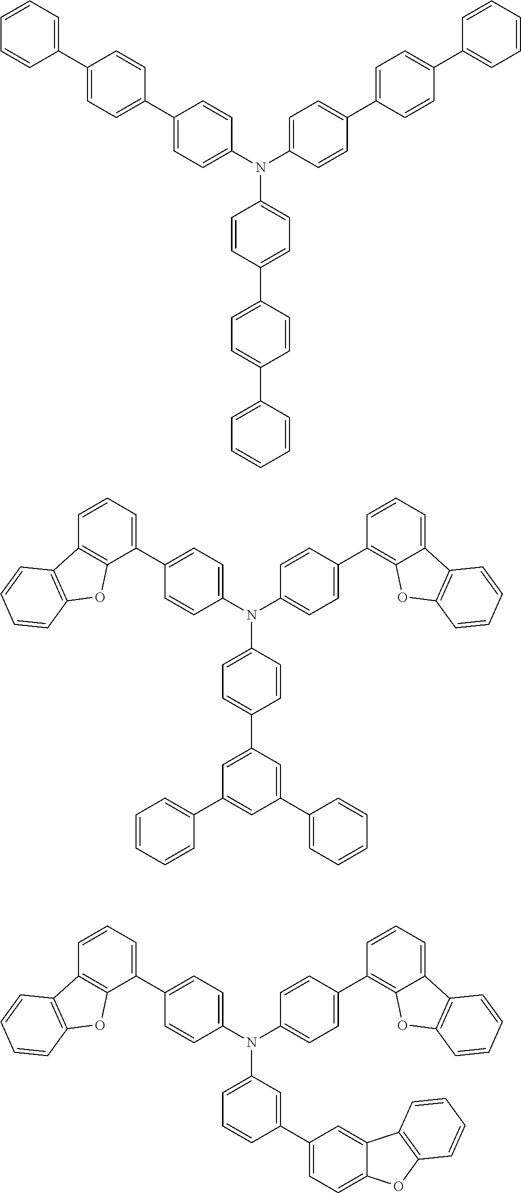 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1 ; c1cc(-c2ccc3oc4ccccc4c3c2)cc(N(c2ccc(-c3cccc4c3oc3ccccc34)cc2)c2ccc(-c3cccc4c3oc3ccccc34)cc2)c1]

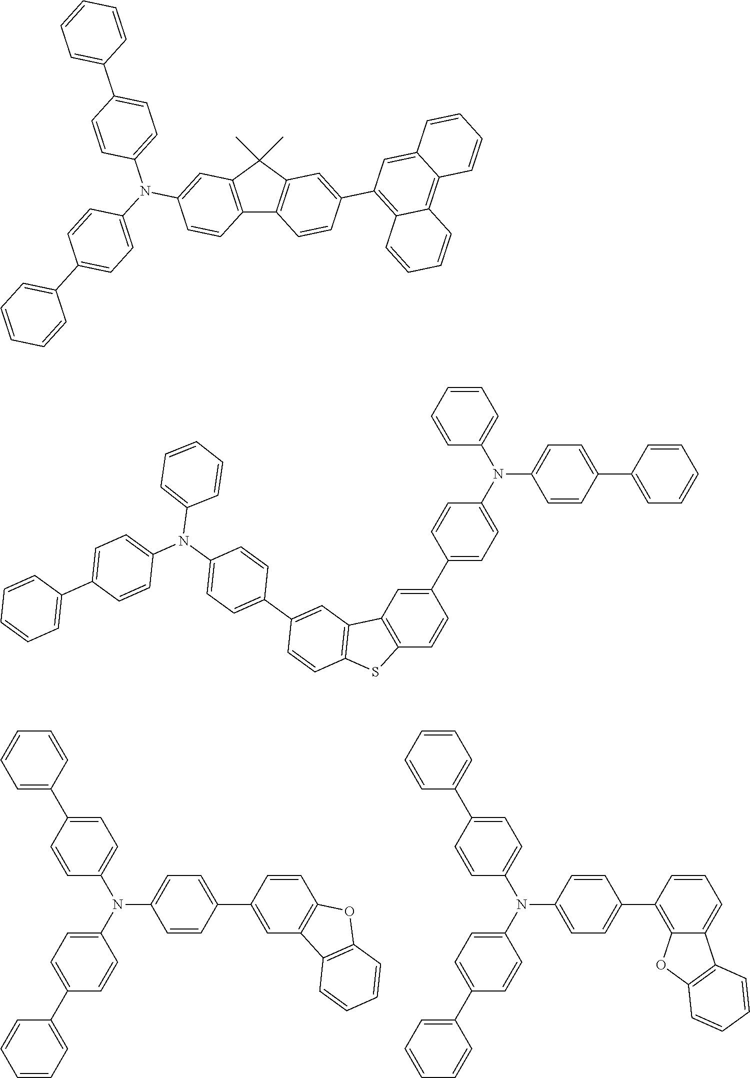 [SMILES: CC1(C)c2cc(-c3cc4ccccc4c4ccccc34)ccc2-c2ccc(N(c3ccc(-c4ccccc4)cc3)c3ccc(-c4ccccc4)cc3)cc21 ; c1ccc(-c2ccc(N(c3ccccc3)c3ccc(-c4ccc5sc6ccc(-c7ccc(N(c8ccccc8)c8ccc(-c9ccccc9)cc8)cc7)cc6c5c4)cc3)cc2)cc1 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3ccc(-c4cccc5c4oc4ccccc45)cc3)cc2)cc1]

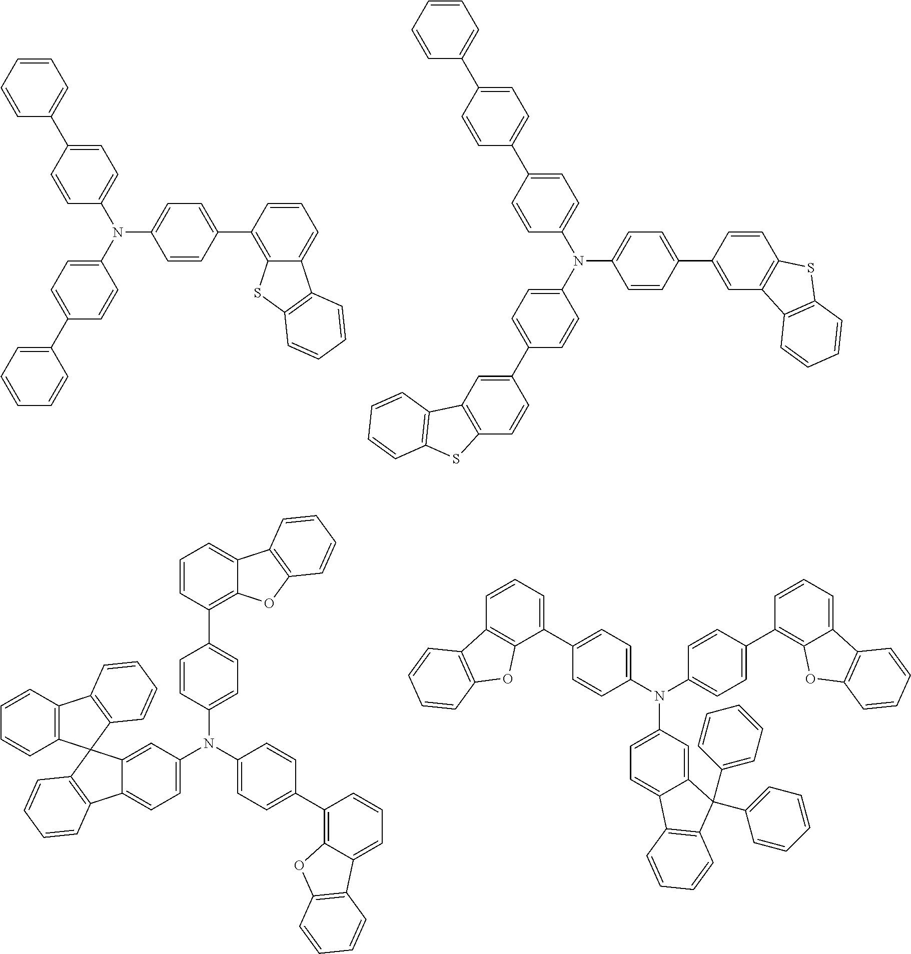 [SMILES: c1ccc(-c2ccc(N(c3ccc(-c4ccccc4)cc3)c3ccc(-c4cccc5c4sc4ccccc45)cc3)cc2)cc1 ; c1ccc(-c2ccc(-c3ccc(N(c4ccc(-c5ccc6sc7ccccc7c6c5)cc4)c4ccc(-c5ccc6sc7ccccc7c6c5)cc4)cc3)cc2)cc1 ; c1ccc2c(c1)-c1ccccc1C21c2ccccc2-c2ccc(N(c3ccc(-c4cccc5c4oc4ccccc45)cc3)c3ccc(-c4cccc5c4oc4ccccc45)cc3)cc21 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1]

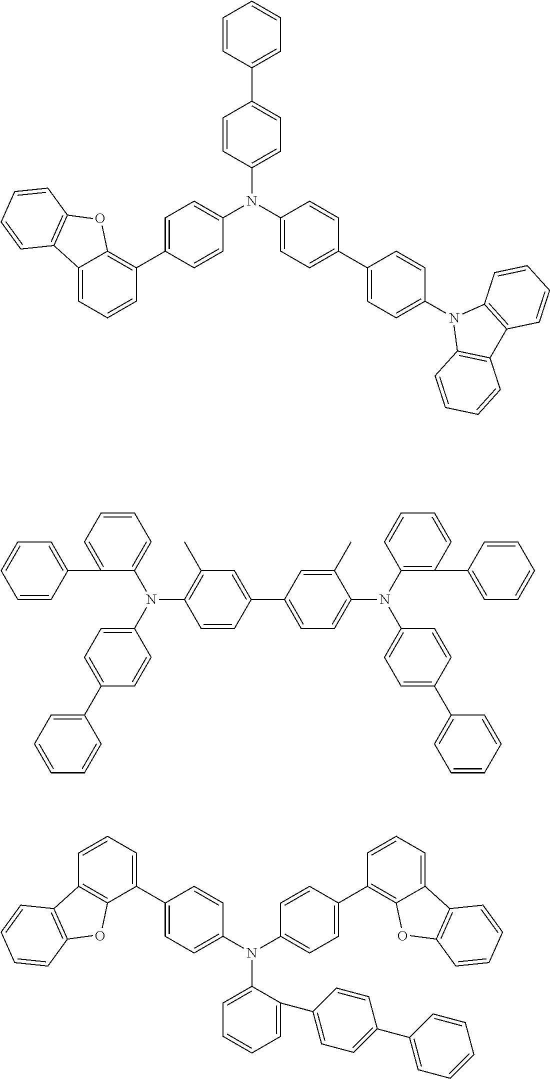 [SMILES: c1ccc(-c2ccc(N(c3ccc(-c4ccc(-n5c6ccccc6c6ccccc65)cc4)cc3)c3ccc(-c4cccc5c4oc4ccccc45)cc3)cc2)cc1 ; Cc1cc(-c2ccc(N(c3ccc(-c4ccccc4)cc3)c3ccccc3-c3ccccc3)c(C)c2)ccc1N(c1ccc(-c2ccccc2)cc1)c1ccccc1-c1ccccc1 ; c1ccc(-c2ccc(-c3ccccc3N(c3ccc(-c4cccc5c4oc4ccccc45)cc3)c3ccc(-c4cccc5c4oc4ccccc45)cc3)cc2)cc1]

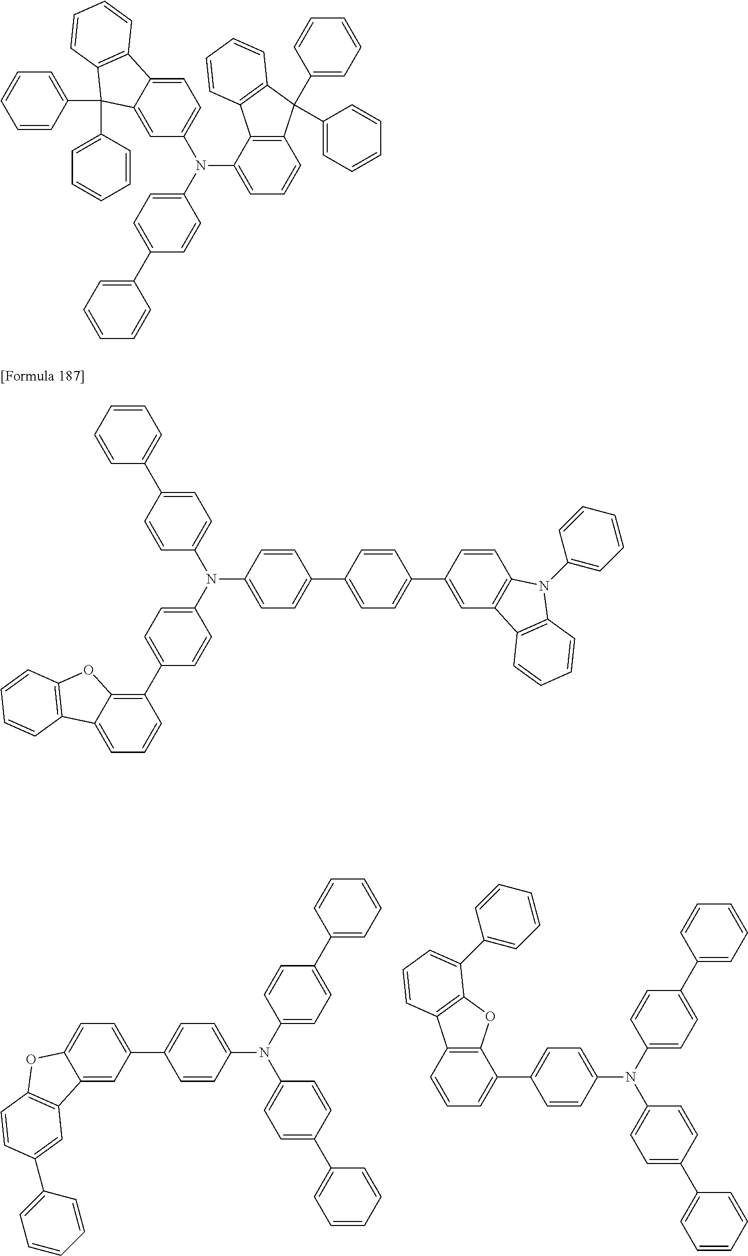 [SMILES: c1ccc(-c2ccc(N(c3ccc4c(c3)C(c3ccccc3)(c3ccccc3)c3ccccc3-4)c3cccc4c3-c3ccccc3C4(c3ccccc3)c3ccccc3)cc2)cc1 ; c1ccc(-c2ccc(N(c3ccc(-c4ccc(-c5ccc6c(c5)c5ccccc5n6-c5ccccc5)cc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5oc6ccc(-c7ccccc7)cc6c5c4)cc3)cc2)cc1 ; c1ccc(-c2ccc(N(c3ccc(-c4ccccc4)cc3)c3ccc(-c4cccc5c4oc4c(-c6ccccc6)cccc45)cc3)cc2)cc1]

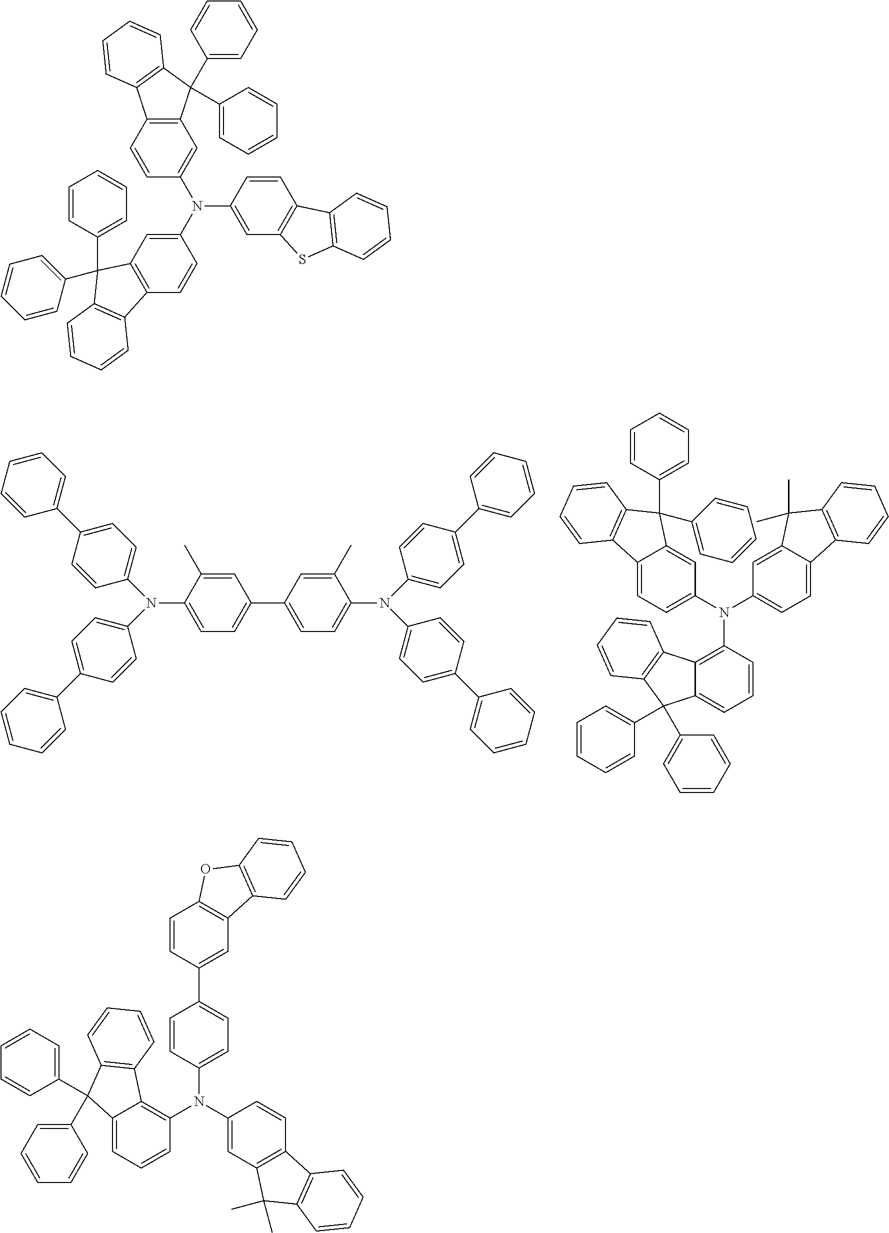

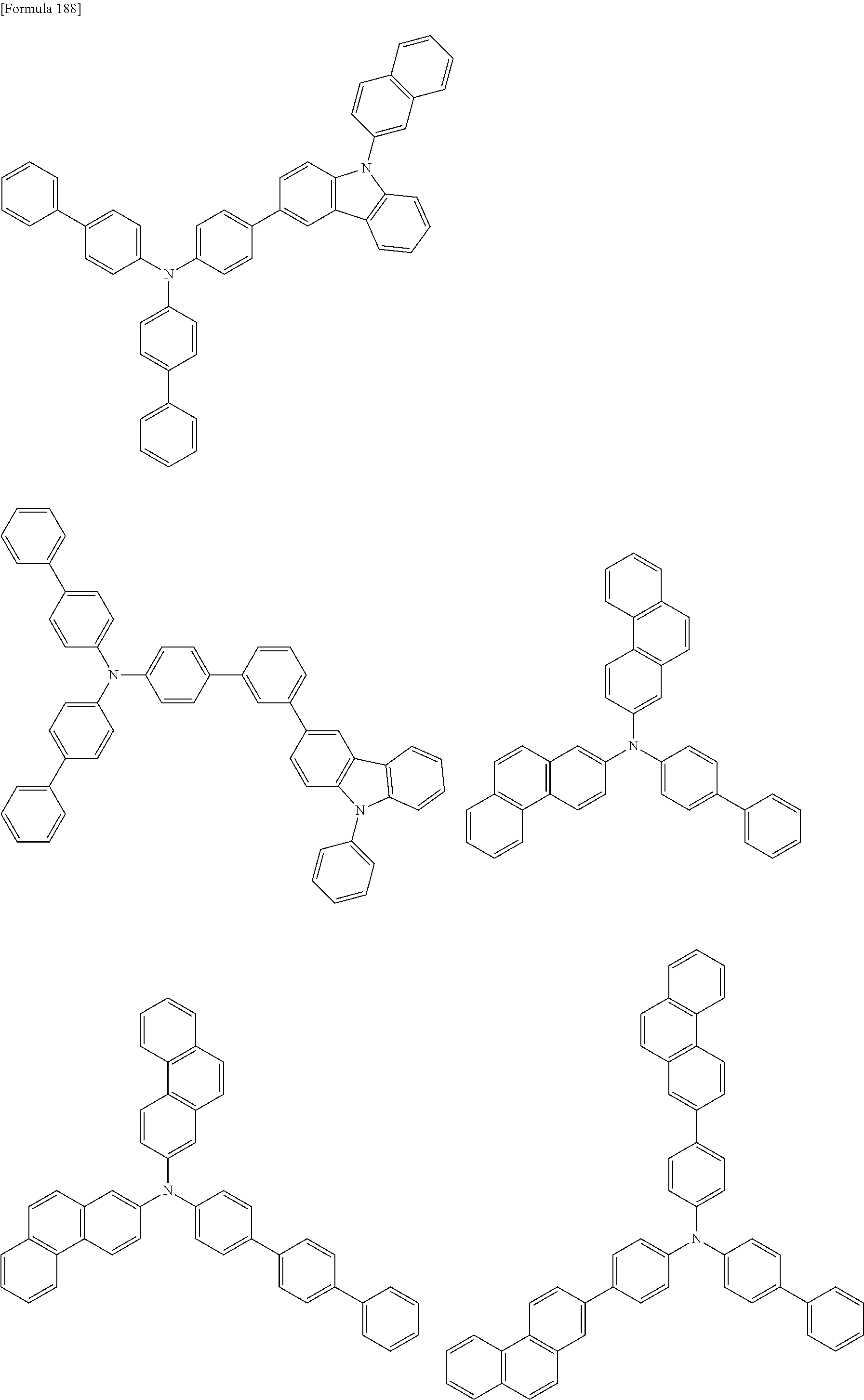

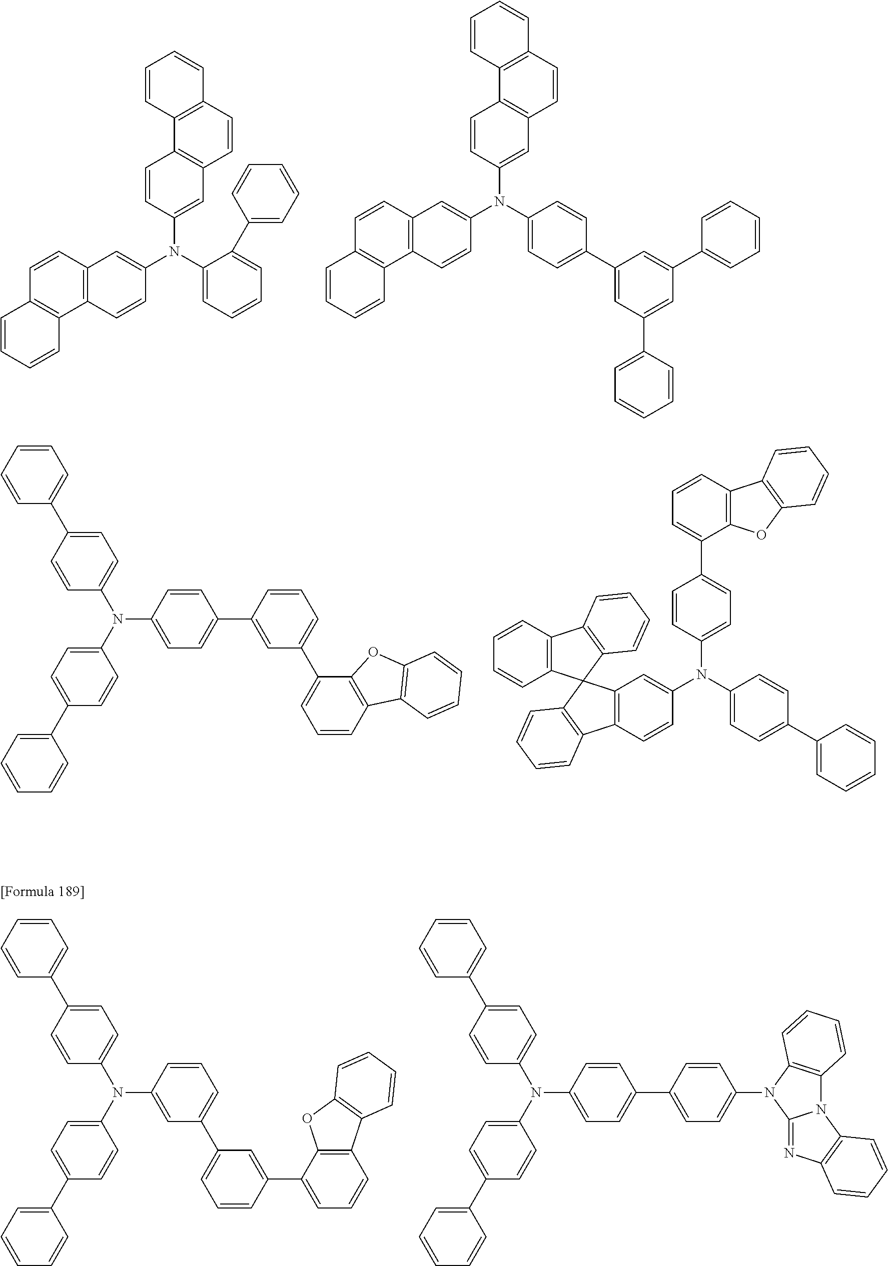

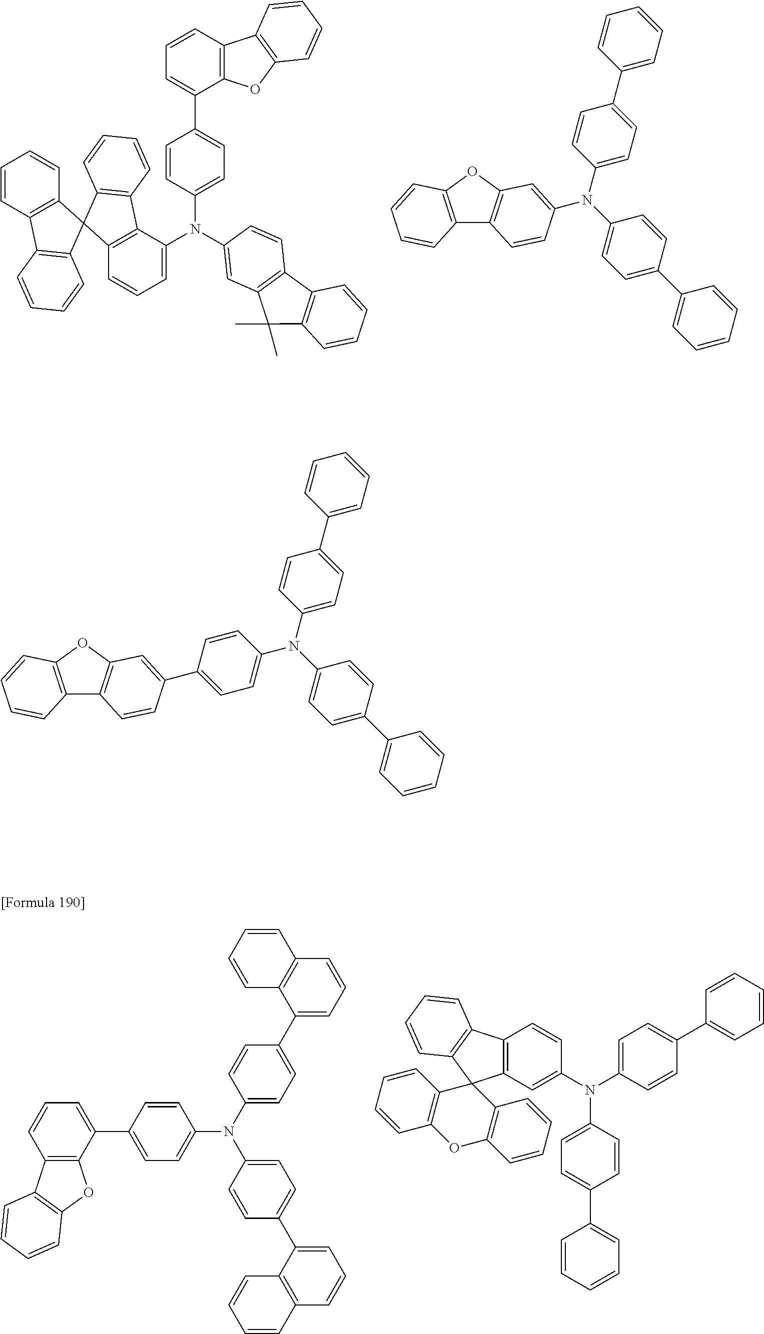 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21 ; c1ccc(-c2ccc(N(c3ccc(-c4ccccc4)cc3)c3ccc4c(c3)oc3ccccc34)cc2)cc1 ; c1ccc(-c2ccc(N(c3ccc(-c4ccccc4)cc3)c3ccc(-c4ccc5c(c4)oc4ccccc45)cc3)cc2)cc1 ; c1ccc2c(-c3ccc(N(c4ccc(-c5cccc6ccccc56)cc4)c4ccc(-c5cccc6c5oc5ccccc56)cc4)cc3)cccc2c1 ; c1ccc(-c2ccc(N(c3ccc(-c4ccccc4)cc3)c3ccc4c(c3)C3(c5ccccc5Oc5ccccc53)c3ccccc3-4)cc2)cc1]

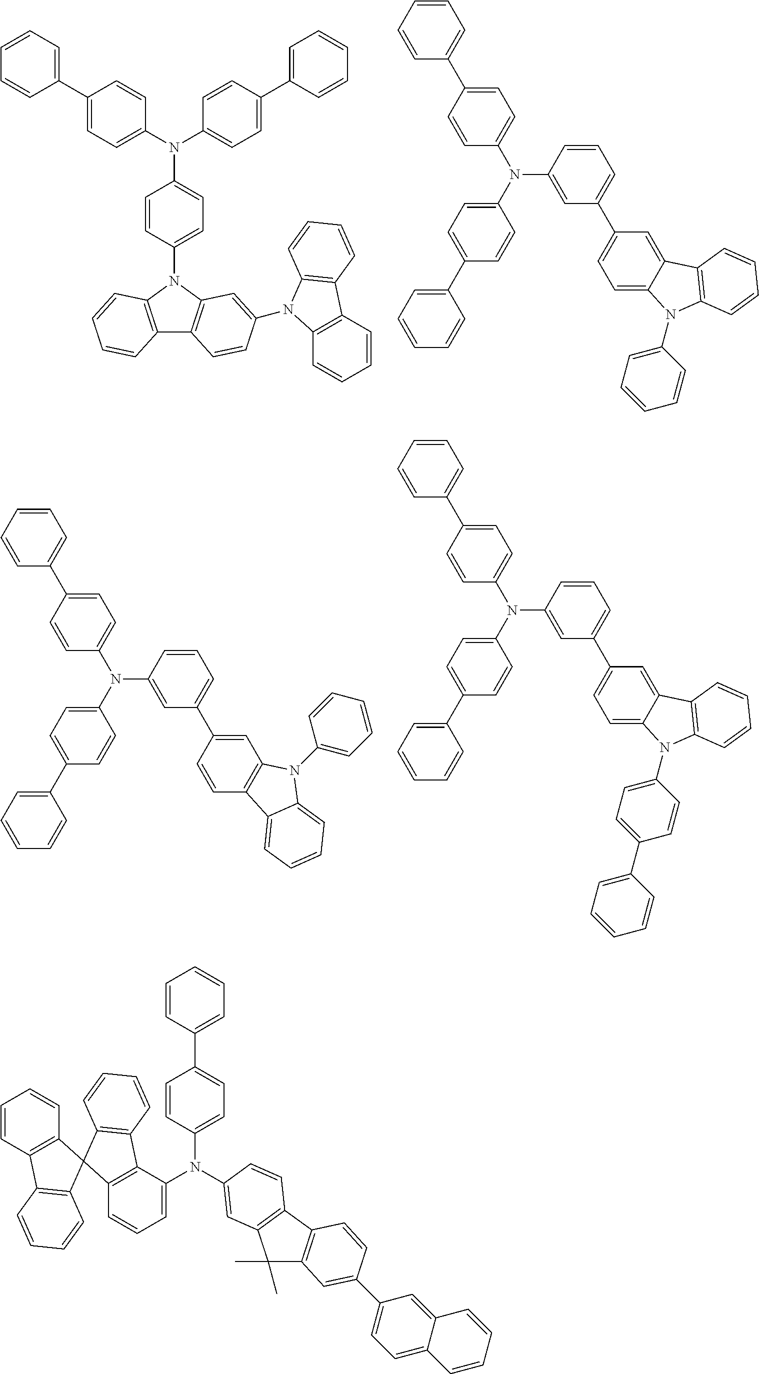

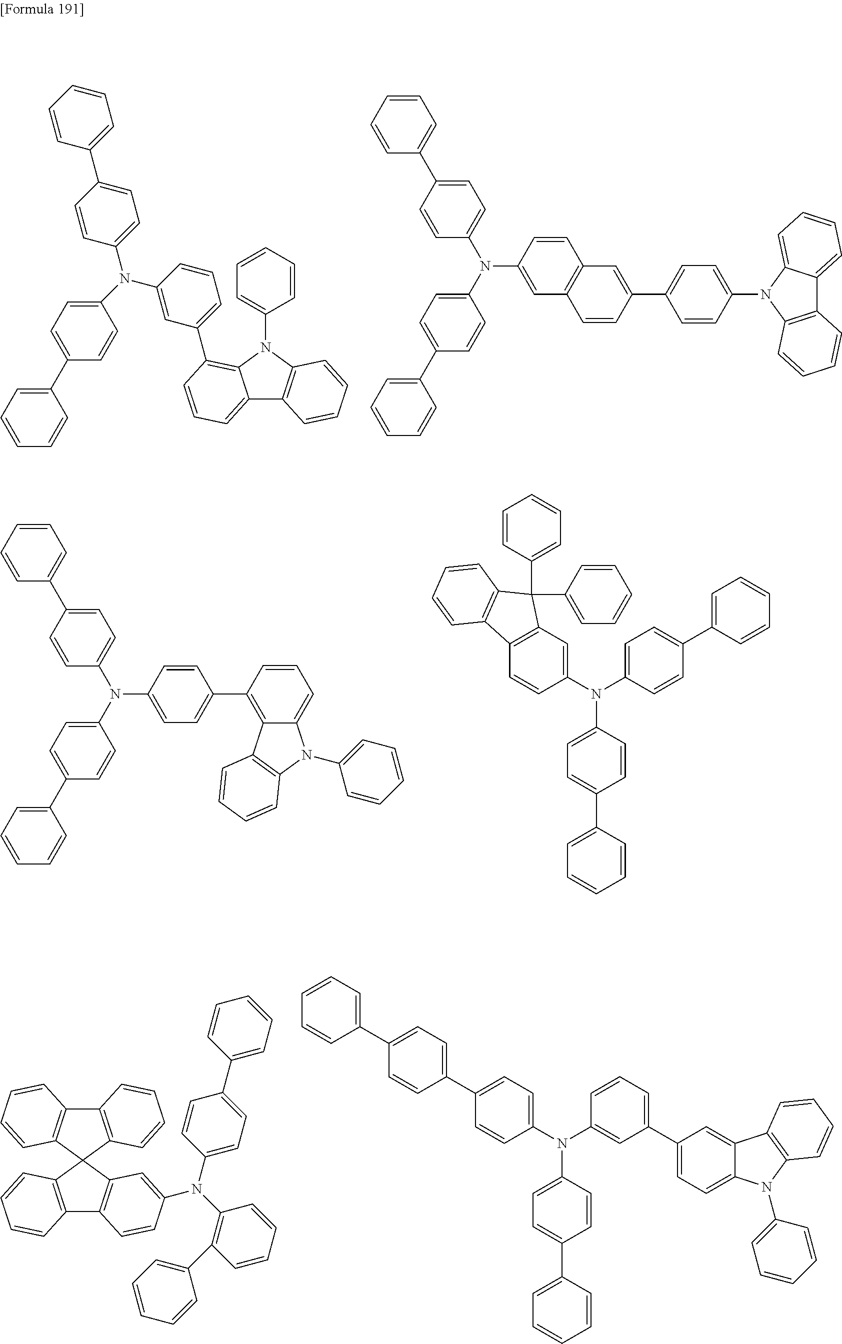

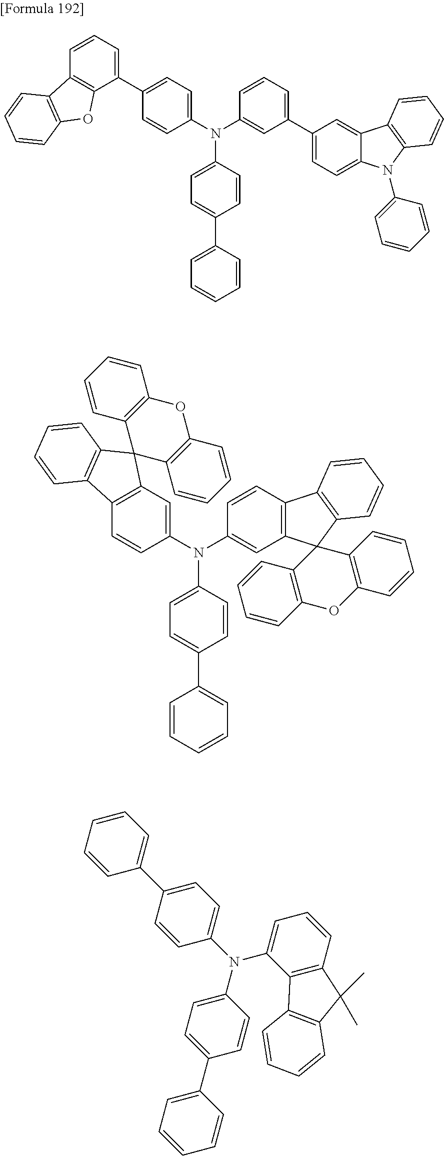 [SMILES: c1ccc(-c2ccc(N(c3ccc(-c4cccc5c4oc4ccccc45)cc3)c3cccc(-c4ccc5c(c4)c4ccccc4n5-c4ccccc4)c3)cc2)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1 ; CC1(C)c2ccccc2-c2c(N(c3ccc(-c4ccccc4)cc3)c3ccc(-c4ccccc4)cc3)cccc21]

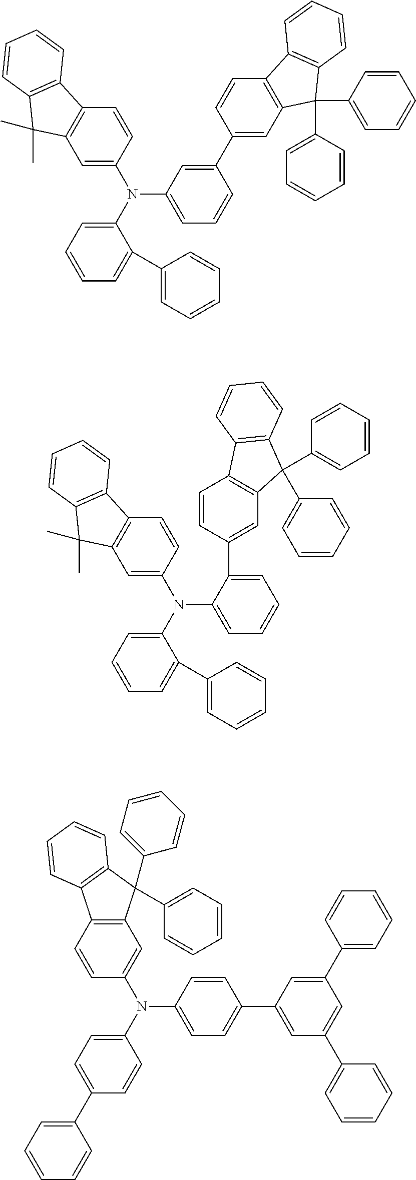 [SMILES: CC1(C)c2ccccc2-c2ccc(N(c3cccc(-c4ccc5c(c4)C(c4ccccc4)(c4ccccc4)c4ccccc4-5)c3)c3ccccc3-c3ccccc3)cc21 ; CC1(C)c2ccccc2-c2ccc(N(c3ccccc3-c3ccccc3)c3ccccc3-c3ccc4c(c3)C(c3ccccc3)(c3ccccc3)c3ccccc3-4)cc21 ; c1ccc(-c2ccc(N(c3ccc(-c4cc(-c5ccccc5)cc(-c5ccccc5)c4)cc3)c3ccc4c(c3)C(c3ccccc3)(c3ccccc3)c3ccccc3-4)cc2)cc1]

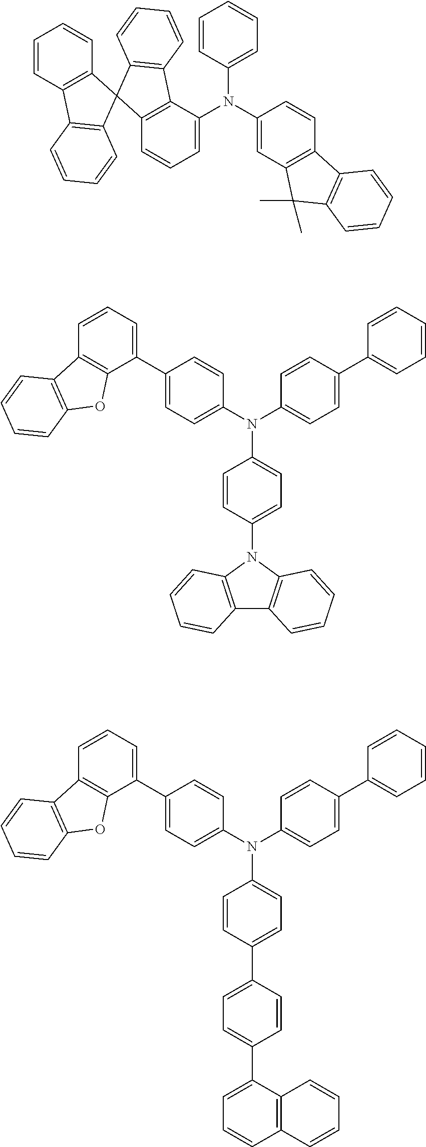 [SMILES: CC1(C)c2ccccc2-c2ccc(N(c3ccccc3)c3cccc4c3-c3ccccc3C43c4ccccc4-c4ccccc43)cc21 ; c1ccc(-c2ccc(N(c3ccc(-c4cccc5c4oc4ccccc45)cc3)c3ccc(-n4c5ccccc5c5ccccc54)cc3)cc2)cc1 ; c1ccc(-c2ccc(N(c3ccc(-c4ccc(-c5cccc6ccccc56)cc4)cc3)c3ccc(-c4cccc5c4oc4ccccc45)cc3)cc2)cc1]

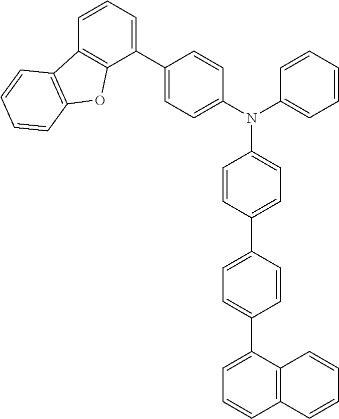 [SMILES: c1ccc(N(c2ccc(-c3ccc(-c4cccc5ccccc45)cc3)cc2)c2ccc(-c3cccc4c3oc3ccccc34)cc2)cc1]

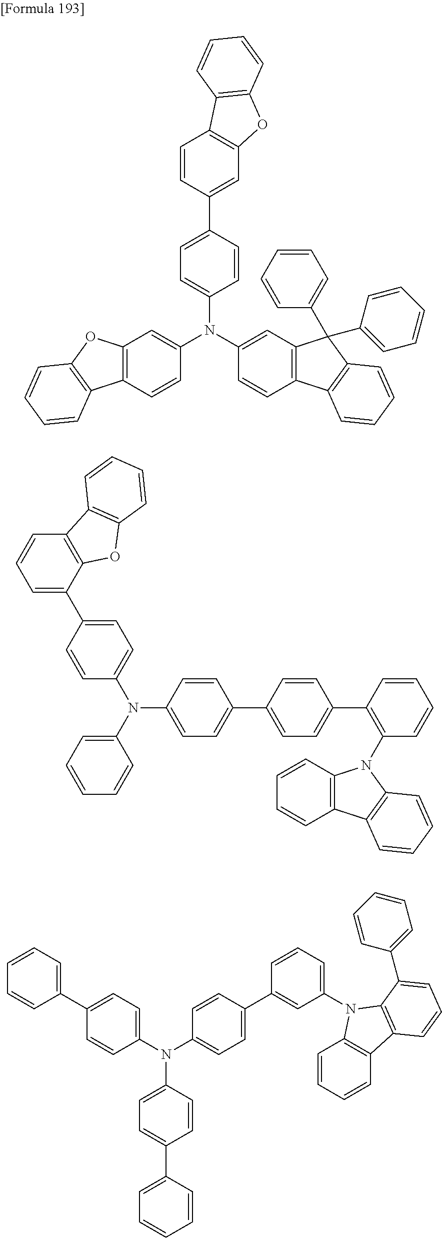

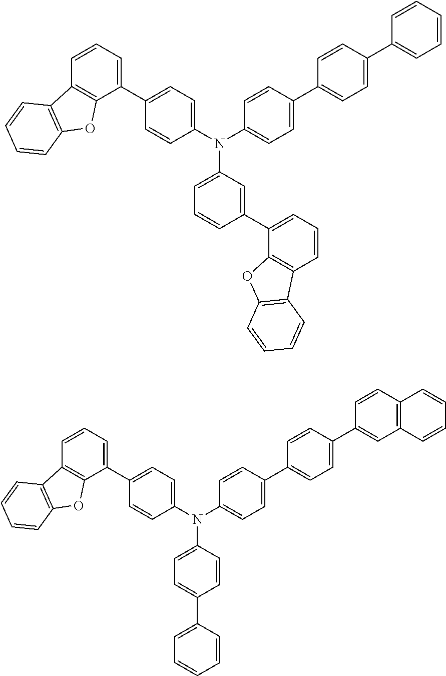 [SMILES: c1ccc(-c2ccc(-c3ccc(N(c4ccc(-c5cccc6c5oc5ccccc56)cc4)c4cccc(-c5cccc6c5oc5ccccc56)c4)cc3)cc2)cc1 ; c1ccc(-c2ccc(N(c3ccc(-c4ccc(-c5ccc6ccccc6c5)cc4)cc3)c3ccc(-c4cccc5c4oc4ccccc45)cc3)cc2)cc1]

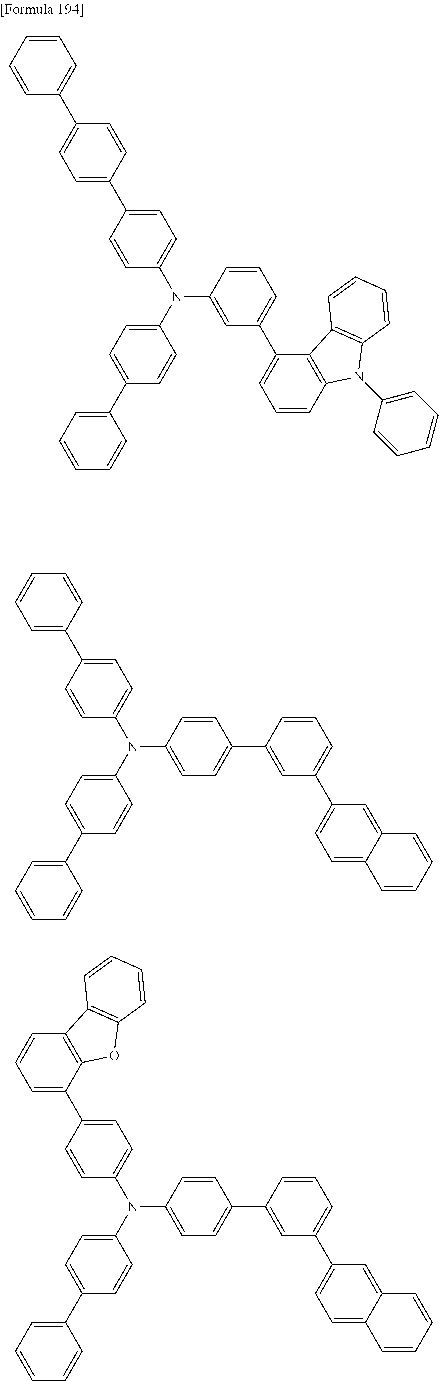 [SMILES: c1ccc(-c2ccc(-c3ccc(N(c4ccc(-c5ccccc5)cc4)c4cccc(-c5cccc6c5c5ccccc5n6-c5ccccc5)c4)cc3)cc2)cc1 ; c1ccc(-c2ccc(N(c3ccc(-c4ccccc4)cc3)c3ccc(-c4cccc(-c5ccc6ccccc6c5)c4)cc3)cc2)cc1 ; c1ccc(-c2ccc(N(c3ccc(-c4cccc(-c5ccc6ccccc6c5)c4)cc3)c3ccc(-c4cccc5c4oc4ccccc45)cc3)cc2)cc1]

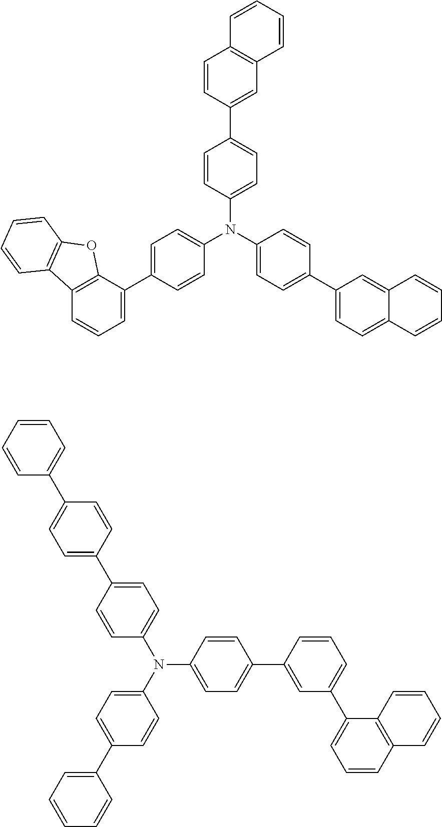 [SMILES: c1ccc2cc(-c3ccc(N(c4ccc(-c5ccc6ccccc6c5)cc4)c4ccc(-c5cccc6c5oc5ccccc56)cc4)cc3)ccc2c1 ; c1ccc(-c2ccc(-c3ccc(N(c4ccc(-c5ccccc5)cc4)c4ccc(-c5cccc(-c6cccc7ccccc67)c5)cc4)cc3)cc2)cc1]

The compound (first organic material) contained in the first anode-side organic layer in an exemplary arrangement of the organic EL devices of the first, second, and third exemplary embodiments as well as the compound (first organic material) contained in the first anode-side organic layer and the compound (second organic material) contained in the second anode-side organic layer in an exemplary arrangement of the organic EL devices of the fourth, fifth, and sixth exemplary embodiments are preferably each independently at least one compound selected from compounds listed below.

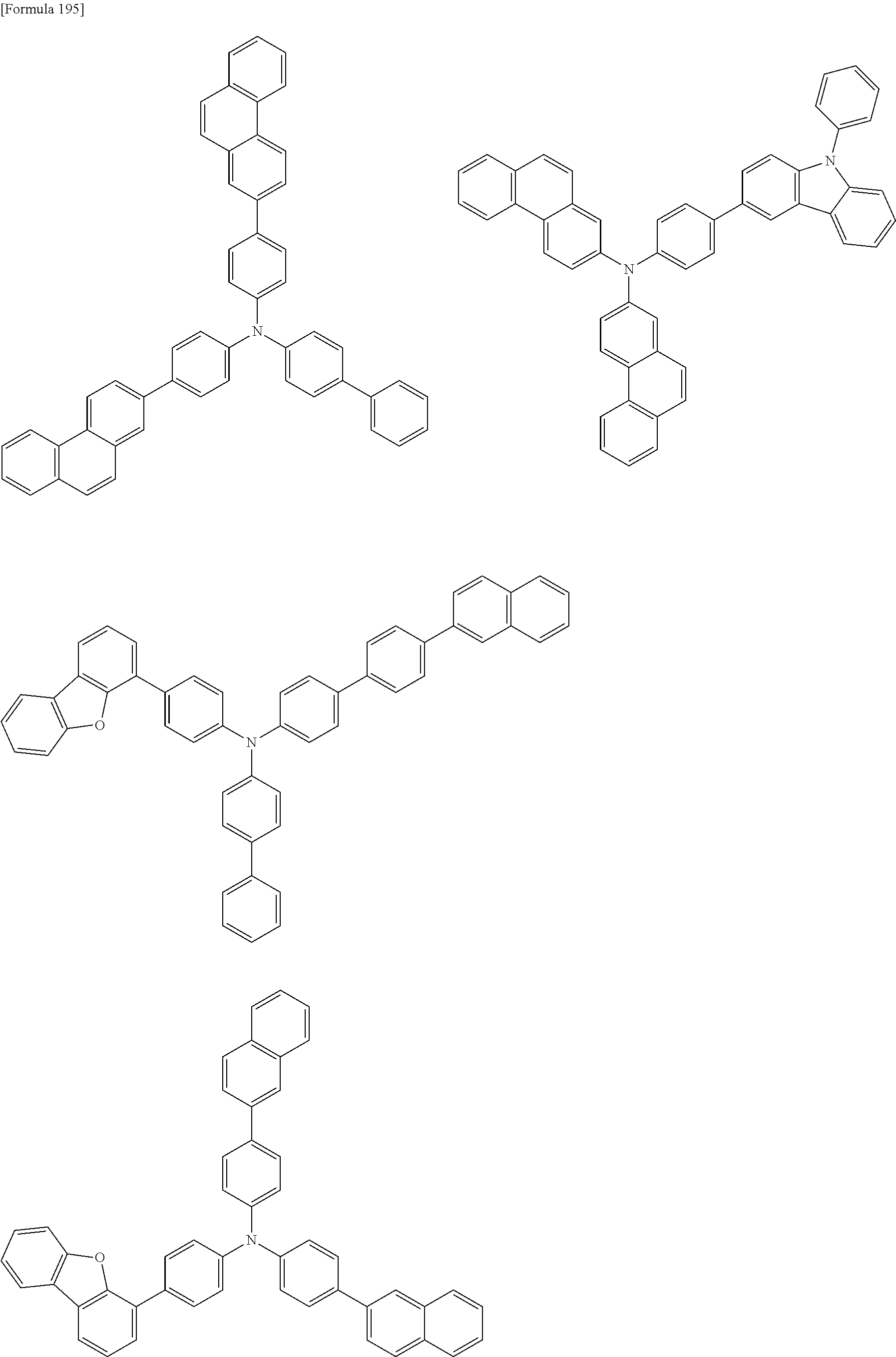 [SMILES: c1ccc(-c2ccc(N(c3ccc(-c4ccc5c(ccc6ccccc65)c4)cc3)c3ccc(-c4ccc5c(ccc6ccccc65)c4)cc3)cc2)cc1 ; c1ccc(-n2c3ccccc3c3cc(-c4ccc(N(c5ccc6c(ccc7ccccc76)c5)c5ccc6c(ccc7ccccc76)c5)cc4)ccc32)cc1 ; c1ccc(-c2ccc(N(c3ccc(-c4ccc(-c5ccc6ccccc6c5)cc4)cc3)c3ccc(-c4cccc5c4oc4ccccc45)cc3)cc2)cc1 ; c1ccc2cc(-c3ccc(N(c4ccc(-c5ccc6ccccc6c5)cc4)c4ccc(-c5cccc6c5oc5ccccc56)cc4)cc3)ccc2c1]

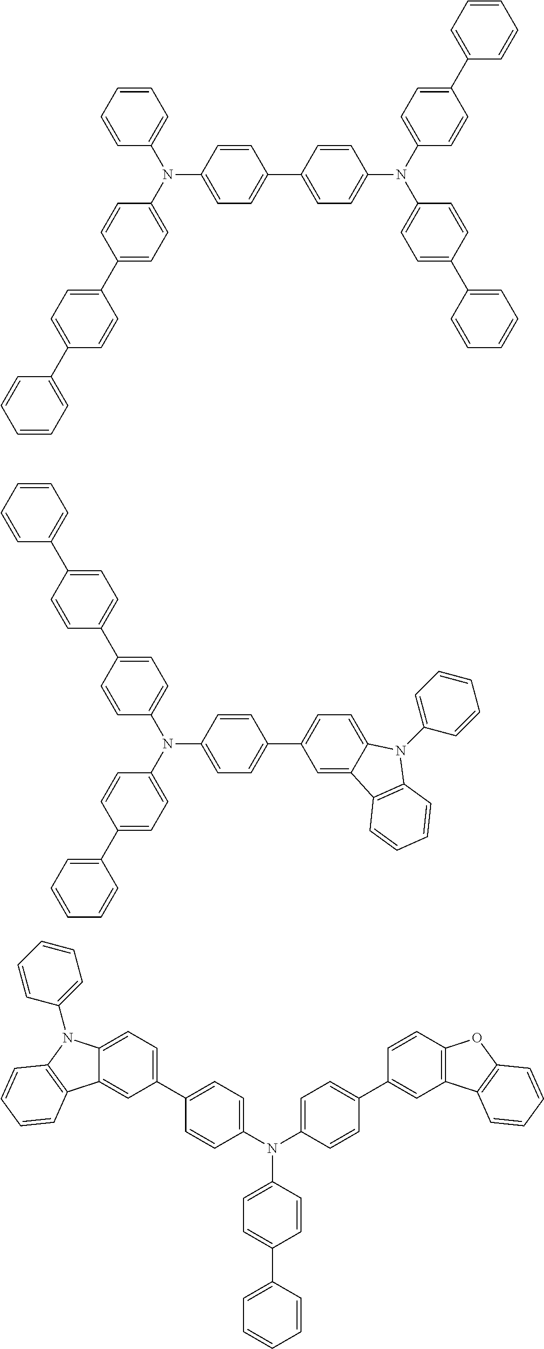

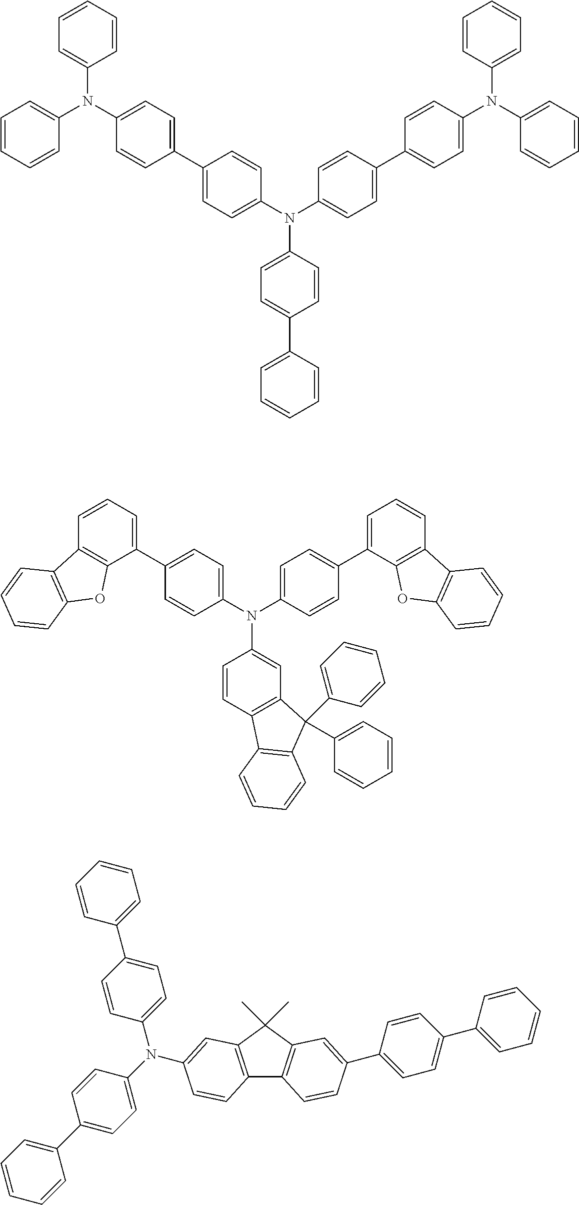 [SMILES: c1ccc(-c2ccc(N(c3ccc(-c4ccc(N(c5ccccc5)c5ccccc5)cc4)cc3)c3ccc(-c4ccc(N(c5ccccc5)c5ccccc5)cc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; CC1(C)c2cc(-c3ccc(-c4ccccc4)cc3)ccc2-c2ccc(N(c3ccc(-c4ccccc4)cc3)c3ccc(-c4ccccc4)cc3)cc21]

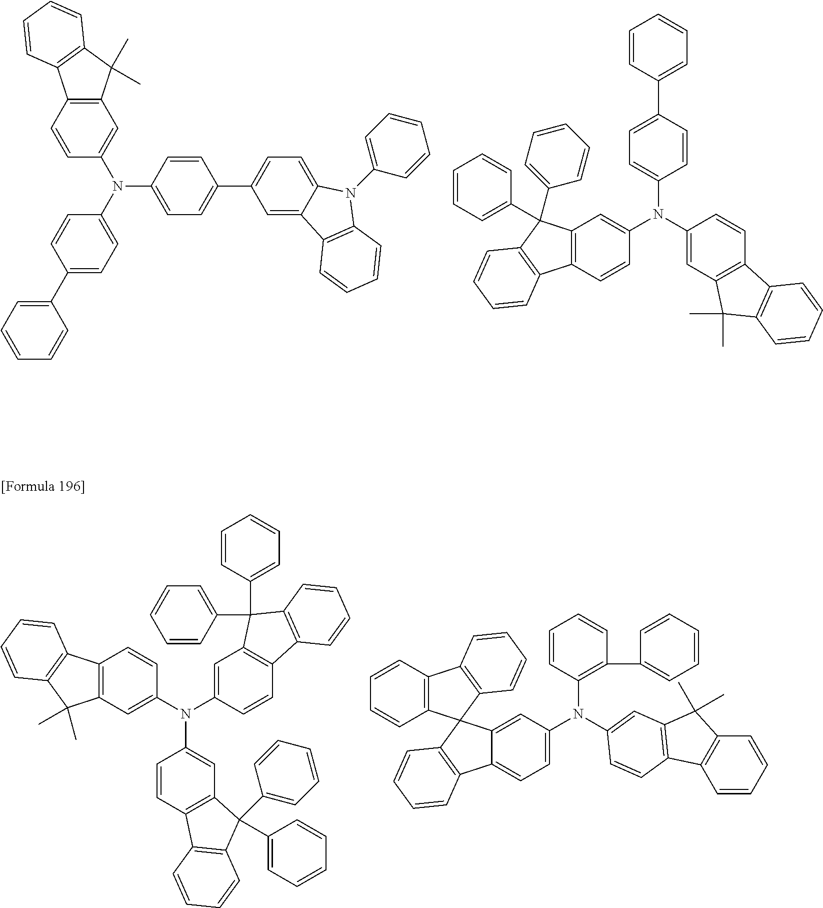

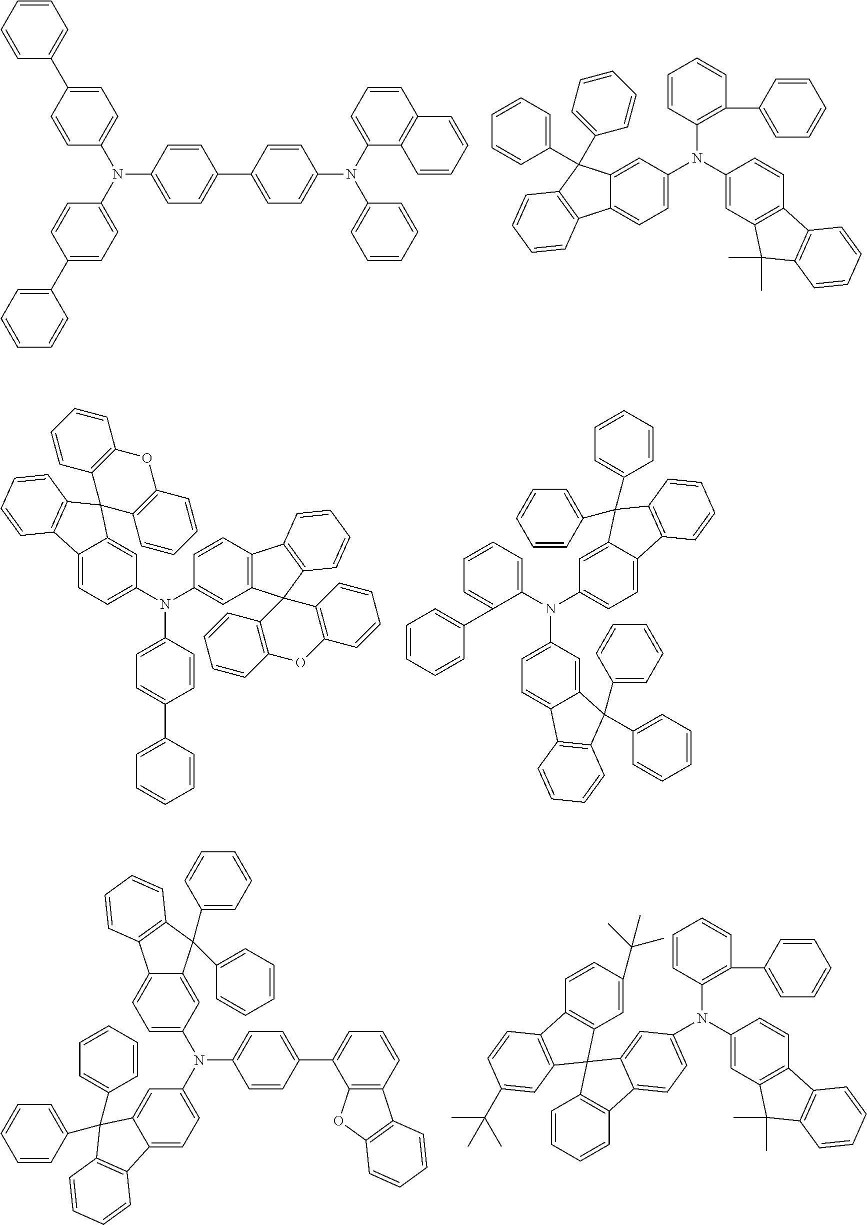

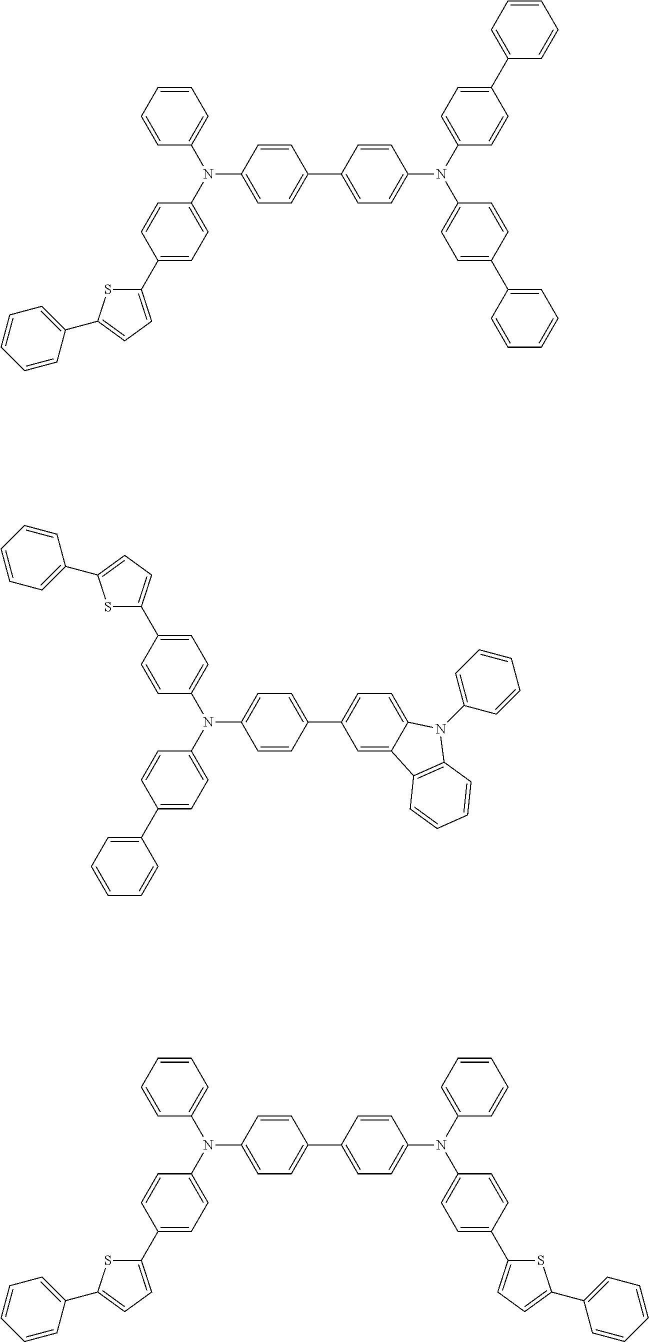 [SMILES: c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1 ; c1ccc(-c2ccc(N(c3ccc(-c4ccc5c(c4)c4ccccc4n5-c4ccccc4)cc3)c3ccc(-c4ccc(-c5ccccc5)s4)cc3)cc2)cc1 ; c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1]

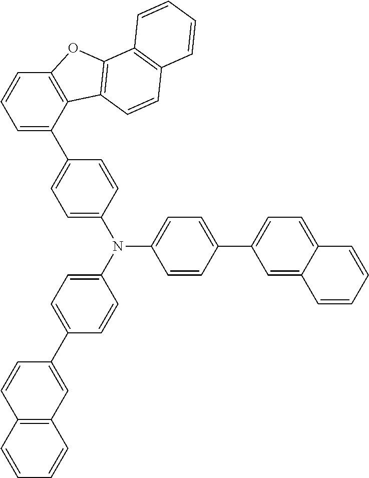 [SMILES: c1ccc2cc(-c3ccc(N(c4ccc(-c5ccc6ccccc6c5)cc4)c4ccc(-c5cccc6oc7c8ccccc8ccc7c56)cc4)cc3)ccc2c1]

The compound (first organic material) contained in the first anode-side organic layer in an exemplary arrangement of the organic EL devices of the first, second, and third exemplary embodiments as well as the compound (first organic material) contained in the first anode-side organic layer and the compound (second organic material) contained in the second anode-side organic layer in an exemplary arrangement of the organic EL devices of the fourth, fifth, and sixth exemplary embodiments are more preferably each independently at least one compound selected from compounds listed below.

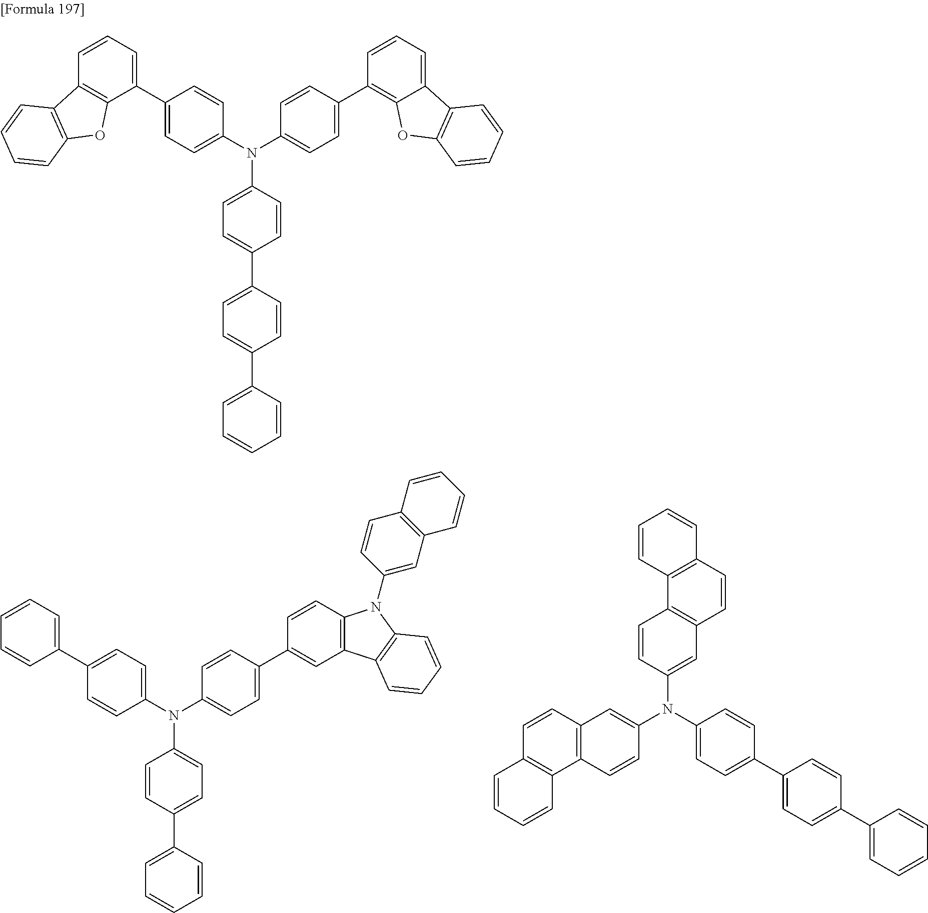 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4ccccc4)cc3)c3ccc(-c4ccc5c(c4)c4ccccc4n5-c4ccc5ccccc5c4)cc3)cc2)cc1 ; c1ccc(-c2ccc(-c3ccc(N(c4ccc5c(ccc6ccccc65)c4)c4ccc5c(ccc6ccccc65)c4)cc3)cc2)cc1]

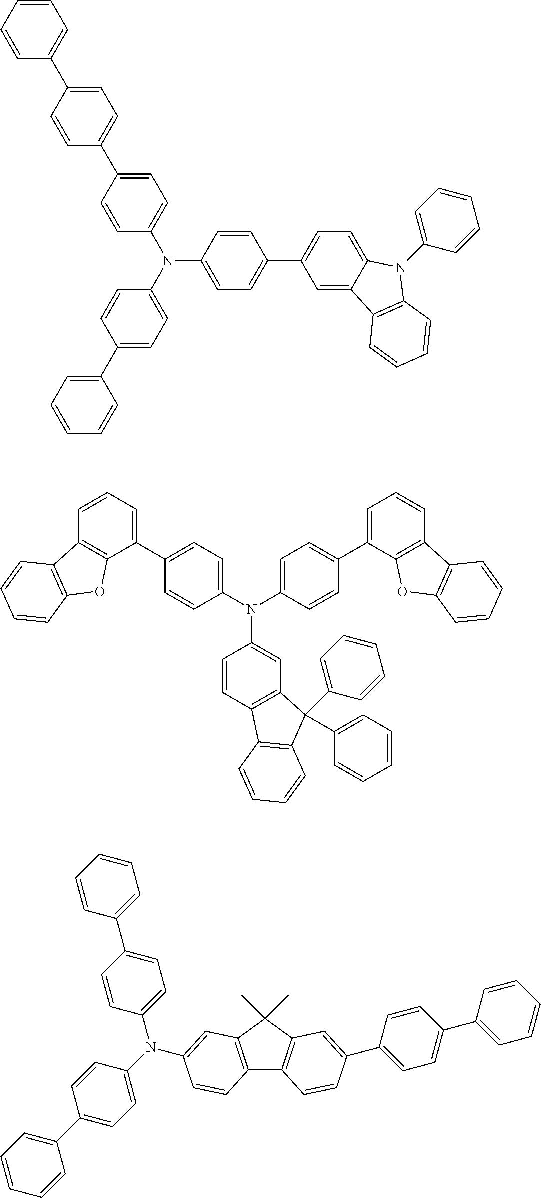 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; CC1(C)c2cc(-c3ccc(-c4ccccc4)cc3)ccc2-c2ccc(N(c3ccc(-c4ccccc4)cc3)c3ccc(-c4ccccc4)cc3)cc21]

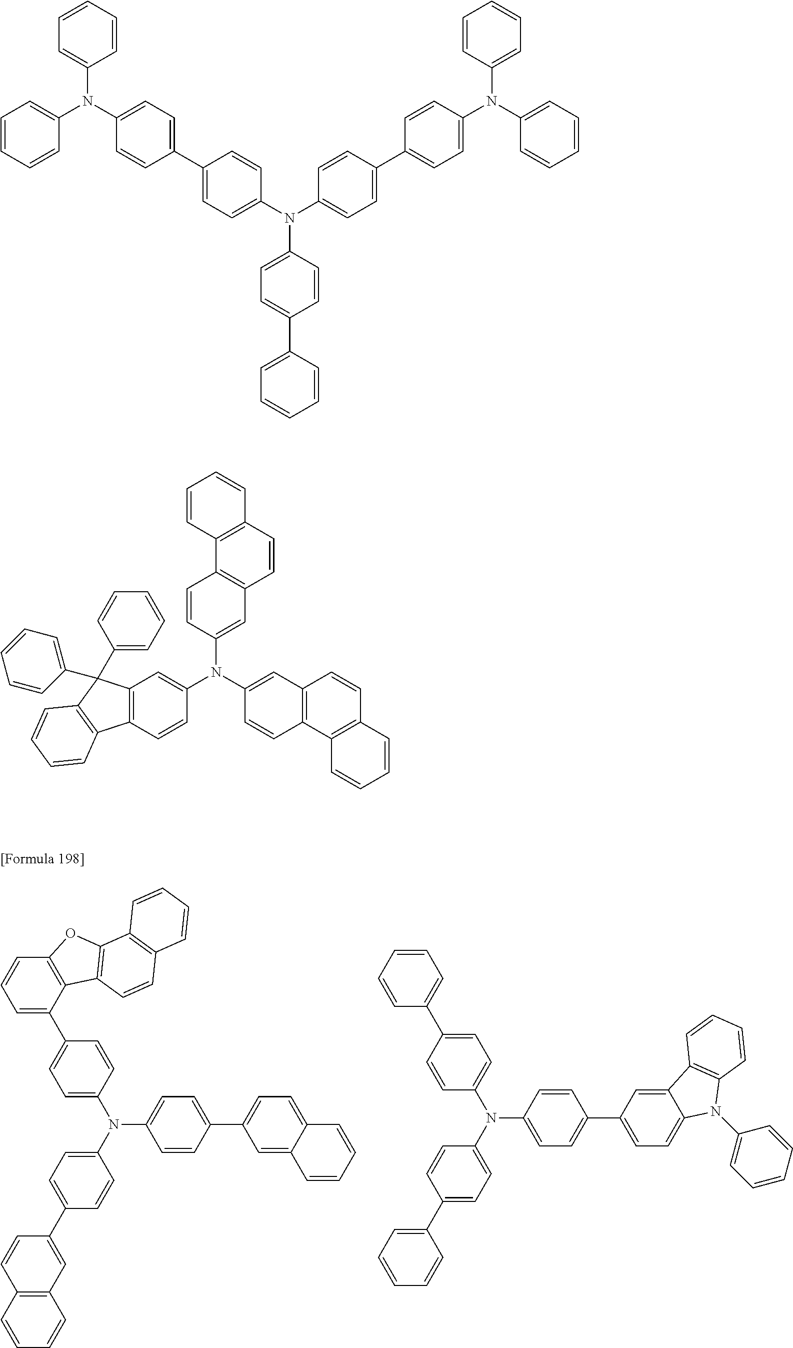 [SMILES: c1ccc(-c2ccc(N(c3ccc(-c4ccc(N(c5ccccc5)c5ccccc5)cc4)cc3)c3ccc(-c4ccc(N(c5ccccc5)c5ccccc5)cc4)cc3)cc2)cc1 ; c1ccc(C2(c3ccccc3)c3ccccc3-c3ccc(N(c4ccc5c(ccc6ccccc65)c4)c4ccc5c(ccc6ccccc65)c4)cc32)cc1 ; c1ccc2cc(-c3ccc(N(c4ccc(-c5ccc6ccccc6c5)cc4)c4ccc(-c5cccc6oc7c8ccccc8ccc7c56)cc4)cc3)ccc2c1 ; c1ccc(-c2ccc(N(c3ccc(-c4ccccc4)cc3)c3ccc(-c4ccc5c(c4)c4ccccc4n5-c4ccccc4)cc3)cc2)cc1]

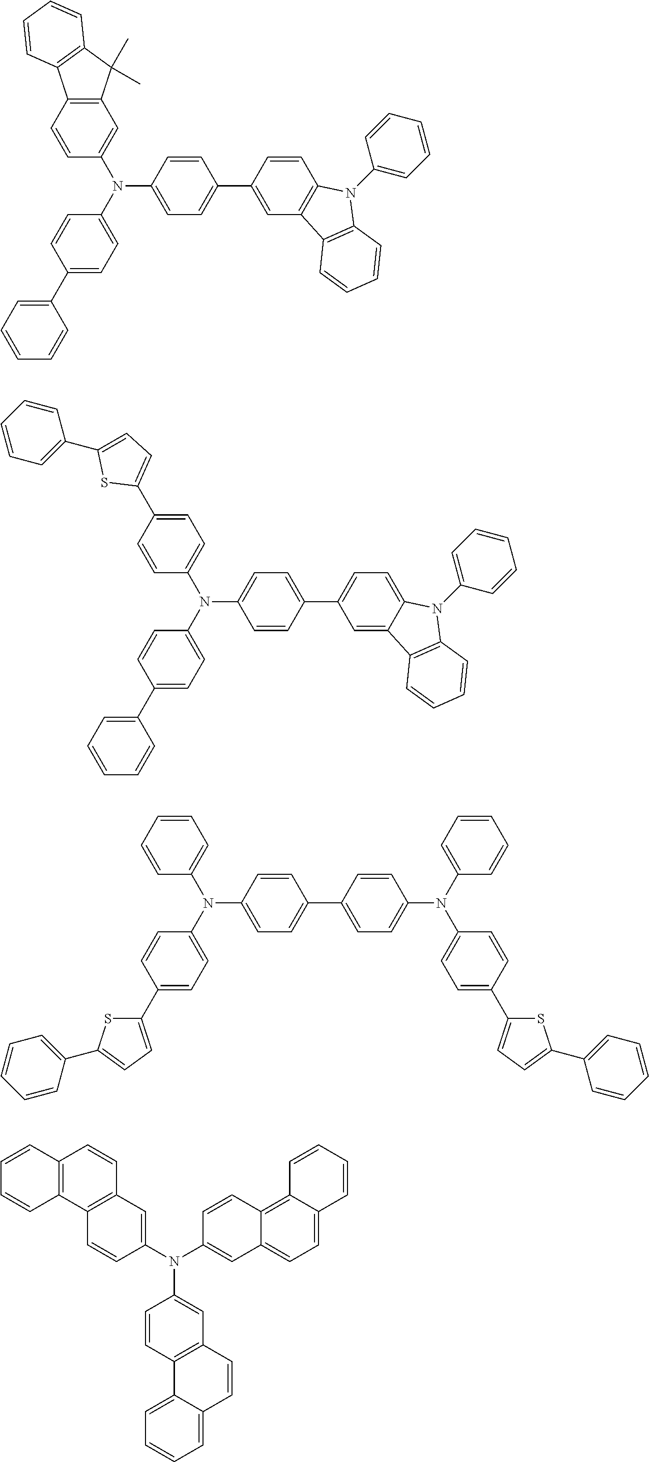

The compound (third organic material) contained in the second anode-side organic layer in an exemplary arrangement of the organic EL devices of the first, second, and third exemplary embodiments as well as the compound (third organic material) contained in the third anode-side organic layer in an exemplary arrangement of the organic EL devices of the fourth, fifth, and sixth exemplary embodiments are preferably each independently at least one compound selected from compounds listed below.

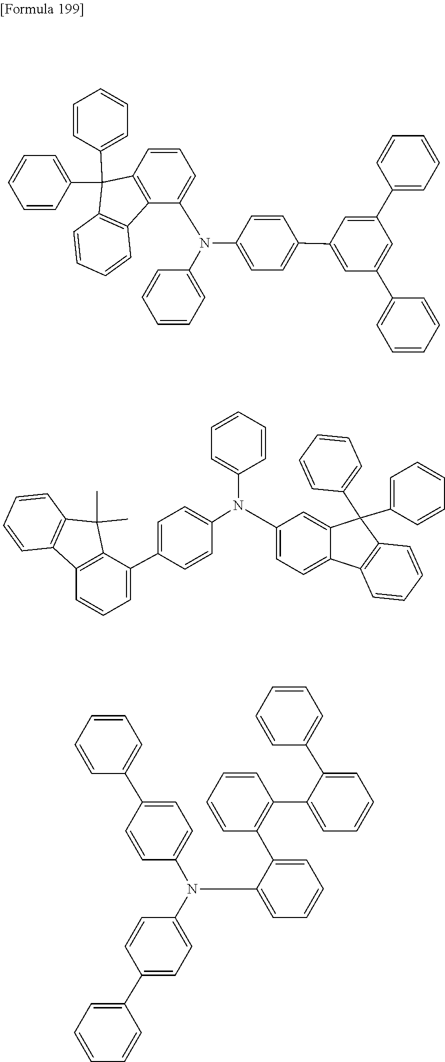 [SMILES: c1ccc(-c2cc(-c3ccccc3)cc(-c3ccc(N(c4ccccc4)c4cccc5c4-c4ccccc4C5(c4ccccc4)c4ccccc4)cc3)c2)cc1 ; CC1(C)c2ccccc2-c2cccc(-c3ccc(N(c4ccccc4)c4ccc5c(c4)C(c4ccccc4)(c4ccccc4)c4ccccc4-5)cc3)c21 ; c1ccc(-c2ccc(N(c3ccc(-c4ccccc4)cc3)c3ccccc3-c3ccccc3-c3ccccc3-c3ccccc3)cc2)cc1]

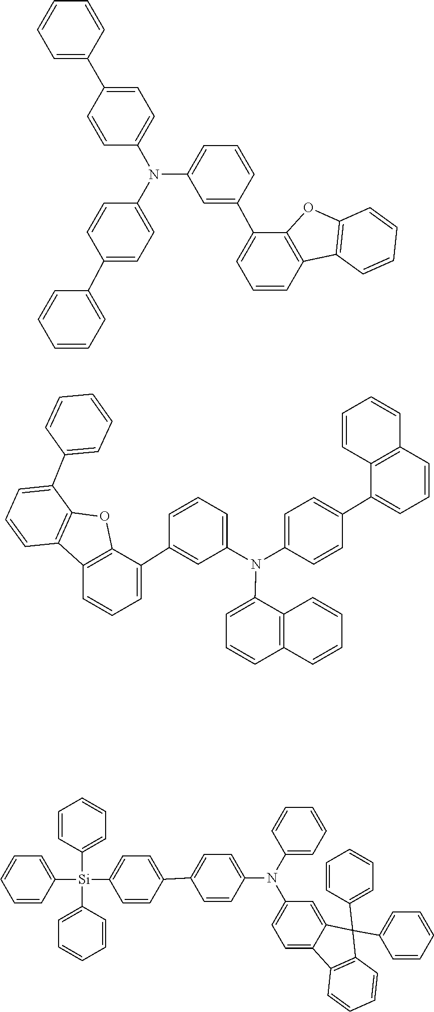

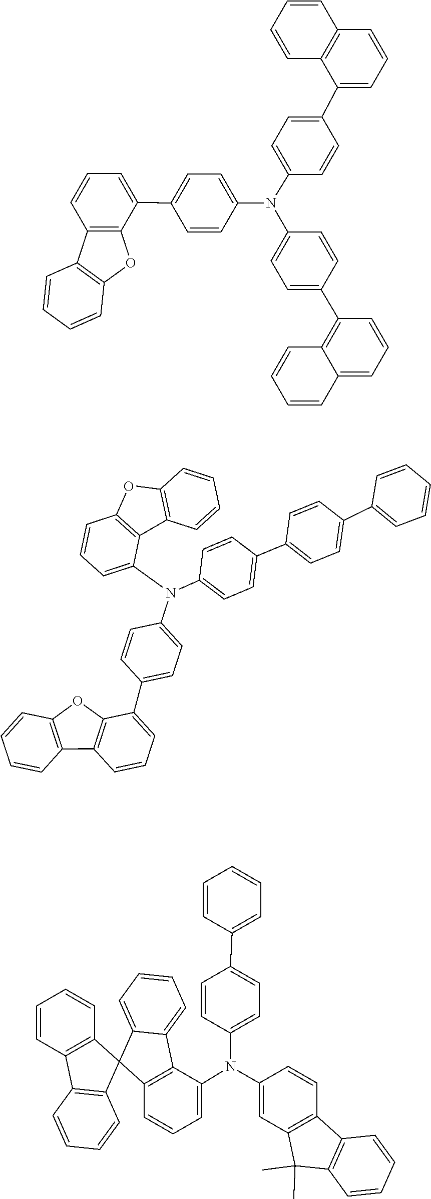 [SMILES: c1ccc2c(-c3ccc(N(c4ccc(-c5cccc6ccccc56)cc4)c4ccc(-c5cccc6c5oc5ccccc56)cc4)cc3)cccc2c1 ; c1ccc(-c2ccc(-c3ccc(N(c4ccc(-c5cccc6c5oc5ccccc56)cc4)c4cccc5oc6ccccc6c45)cc3)cc2)cc1 ; CC1(C)c2ccccc2-c2ccc(N(c3ccc(-c4ccccc4)cc3)c3cccc4c3-c3ccccc3C43c4ccccc4-c4ccccc43)cc21]

The compound (third organic material) contained in the second anode-side organic layer in an exemplary arrangement of the organic EL devices of the first, second, and third exemplary embodiments as well as the compound (third organic material) contained in the third anode-side organic layer in an exemplary arrangement of the organic EL devices of the fourth, fifth, and sixth exemplary embodiments are more preferably each independently at least one compound selected from compounds listed below.

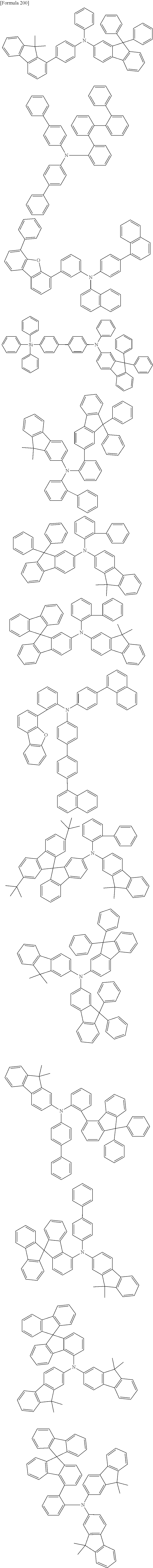

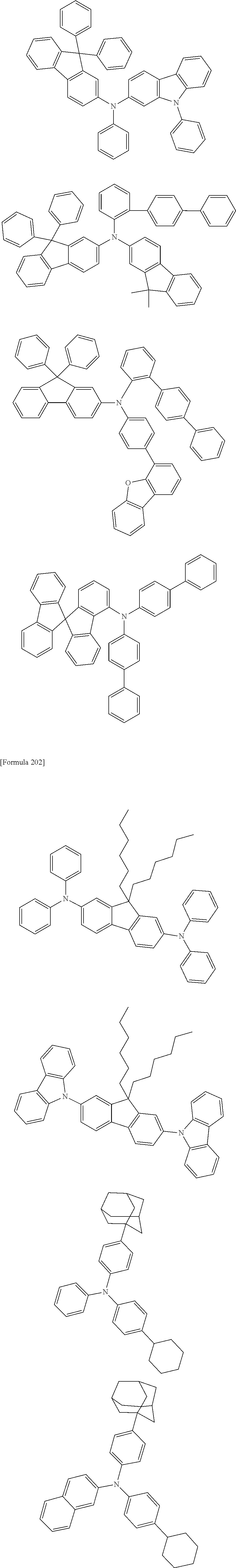

In the first, second, and third exemplary embodiments, exemplary compounds listed as the compounds contained in one of the first anode-side organic layer and the second anode-side organic layer may overlap with exemplary compounds contained in the other of the first anode-side organic layer and the second anode-side organic layer; however, in the first, second, and third exemplary embodiments, as compounds usable in the first anode-side organic layer and the second anode-side organic layer, mutually different compounds can be appropriately selected from the exemplary compounds.

In the fourth, fifth, and sixth exemplary embodiments, the exemplary compounds listed as the compounds contained in one of the first anode-side organic layer, the second anode-side organic layer, and the third anode-side organic layer may be listed overlapping with exemplary compounds contained in the rest of the first anode-side organic layer, the second anode-side organic layer, and the third anode-side organic layer. However, mutually different compounds may be appropriately selected from the exemplary compounds as compounds usable in the first anode-side organic layer, the second anode-side organic layer, and the third anode-side organic layer in the fourth, fifth, and sixth exemplary embodiments.

### Specific Examples of Second Organic Material and Acceptor Material

Specific examples of the second organic material in the first, second, and third exemplary embodiments and the acceptor material in the fourth, fifth, and sixth exemplary embodiments include compounds below. However, the invention is by no means limited to the specific examples of the second organic material and the acceptor material.

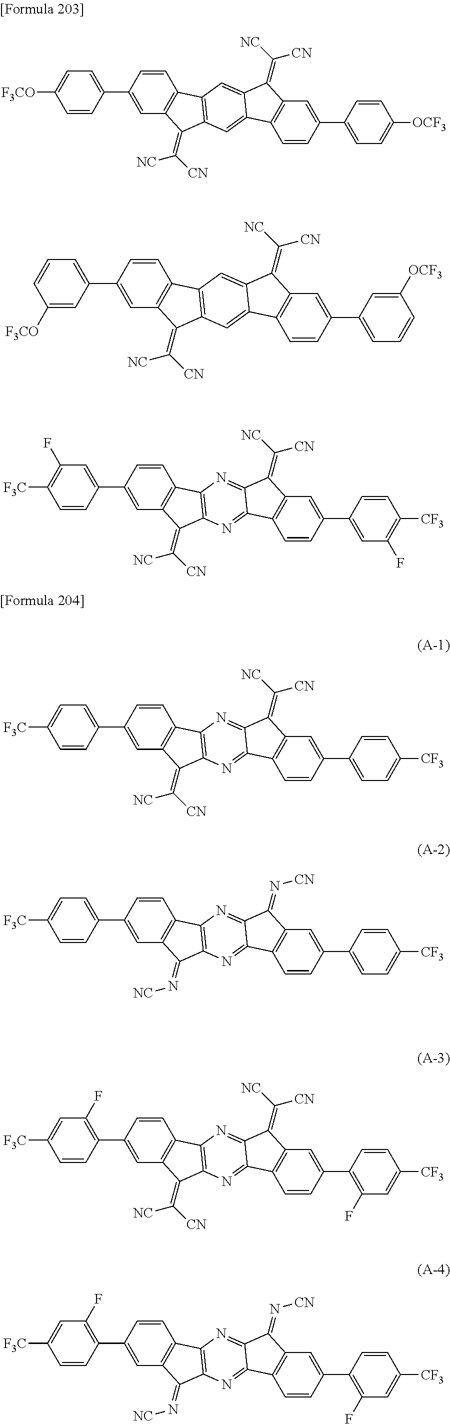

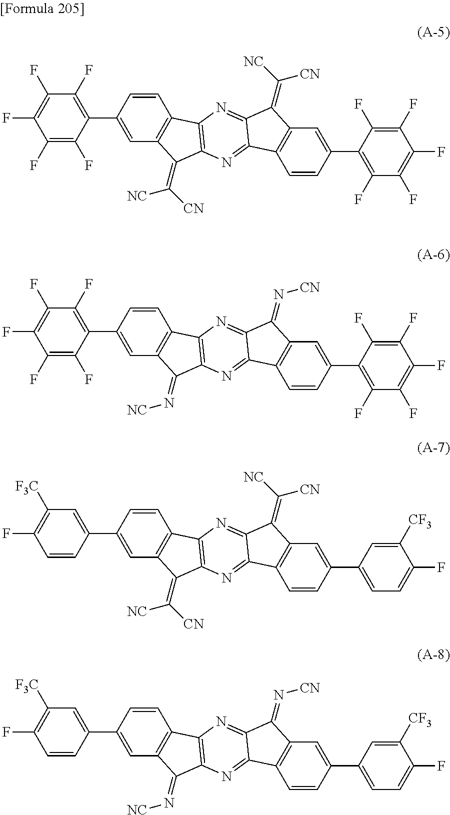 [SMILES: N#CC(C#N)=C1c2cc(-c3c(F)c(F)c(F)c(F)c3F)ccc2-c2nc3c(nc21)-c1ccc(-c2c(F)c(F)c(F)c(F)c2F)cc1C3=C(C#N)C#N ; N#C/N=c1\c2cc(-c3c(F)c(F)c(F)c(F)c3F)ccc2c2nc3/c(=N/C#N)c4cc(-c5c(F)c(F)c(F)c(F)c5F)ccc4c3nc12 ; N#CC(C#N)=C1c2cc(-c3ccc(F)c(C(F)(F)F)c3)ccc2-c2nc3c(nc21)-c1ccc(-c2ccc(F)c(C(F)(F)F)c2)cc1C3=C(C#N)C#N ; N#C/N=c1\c2cc(-c3ccc(F)c(C(F)(F)F)c3)ccc2c2nc3/c(=N/C#N)c4cc(-c5ccc(F)c(C(F)(F)F)c5)ccc4c3nc12]

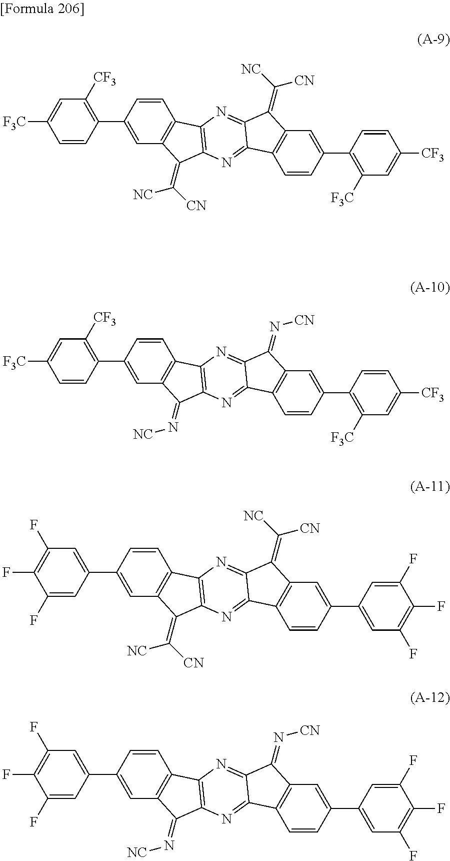 [SMILES: N#CC(C#N)=C1c2cc(-c3ccc(C(F)(F)F)cc3C(F)(F)F)ccc2-c2nc3c(nc21)-c1ccc(-c2ccc(C(F)(F)F)cc2C(F)(F)F)cc1C3=C(C#N)C#N ; N#C/N=c1\c2cc(-c3ccc(C(F)(F)F)cc3C(F)(F)F)ccc2c2nc3/c(=N/C#N)c4cc(-c5ccc(C(F)(F)F)cc5C(F)(F)F)ccc4c3nc12 ; N#CC(C#N)=C1c2cc(-c3cc(F)c(F)c(F)c3)ccc2-c2nc3c(nc21)-c1ccc(-c2cc(F)c(F)c(F)c2)cc1C3=C(C#N)C#N ; N#C/N=c1\c2cc(-c3cc(F)c(F)c(F)c3)ccc2c2nc3/c(=N/C#N)c4cc(-c5cc(F)c(F)c(F)c5)ccc4c3nc12]

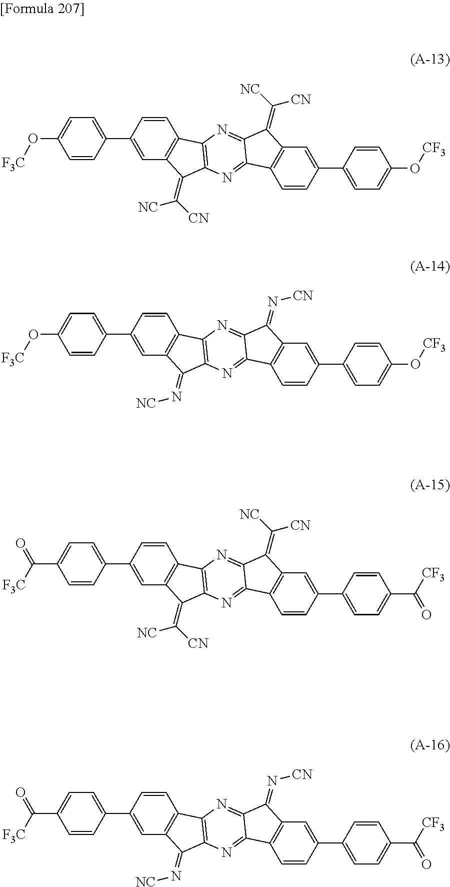 [SMILES: N#CC(C#N)=C1c2cc(-c3ccc(OC(F)(F)F)cc3)ccc2-c2nc3c(nc21)-c1ccc(-c2ccc(OC(F)(F)F)cc2)cc1C3=C(C#N)C#N ; N#C/N=c1\c2cc(-c3ccc(OC(F)(F)F)cc3)ccc2c2nc3/c(=N/C#N)c4cc(-c5ccc(OC(F)(F)F)cc5)ccc4c3nc12 ; N#CC(C#N)=C1c2cc(-c3ccc(C(=O)C(F)(F)F)cc3)ccc2-c2nc3c(nc21)-c1ccc(-c2ccc(C(=O)C(F)(F)F)cc2)cc1C3=C(C#N)C#N ; N#C/N=c1\c2cc(-c3ccc(C(=O)C(F)(F)F)cc3)ccc2c2nc3/c(=N/C#N)c4cc(-c5ccc(C(=O)C(F)(F)F)cc5)ccc4c3nc12]

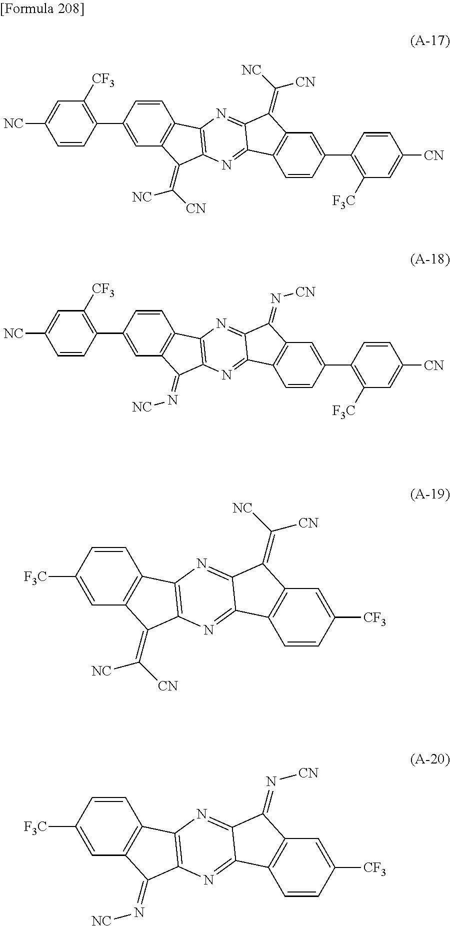 [SMILES: N#CC(C#N)=C1c2cc(-c3ccc(C#N)cc3C(F)(F)F)ccc2-c2nc3c(nc21)-c1ccc(-c2ccc(C#N)cc2C(F)(F)F)cc1C3=C(C#N)C#N ; N#C/N=c1\c2cc(-c3ccc(C#N)cc3C(F)(F)F)ccc2c2nc3/c(=N/C#N)c4cc(-c5ccc(C#N)cc5C(F)(F)F)ccc4c3nc12 ; N#CC(C#N)=C1c2cc(C(F)(F)F)ccc2-c2nc3c(nc21)-c1ccc(C(F)(F)F)cc1C3=C(C#N)C#N ; N#C/N=c1\c2cc(C(F)(F)F)ccc2c2nc3/c(=N/C#N)c4cc(C(F)(F)F)ccc4c3nc12]

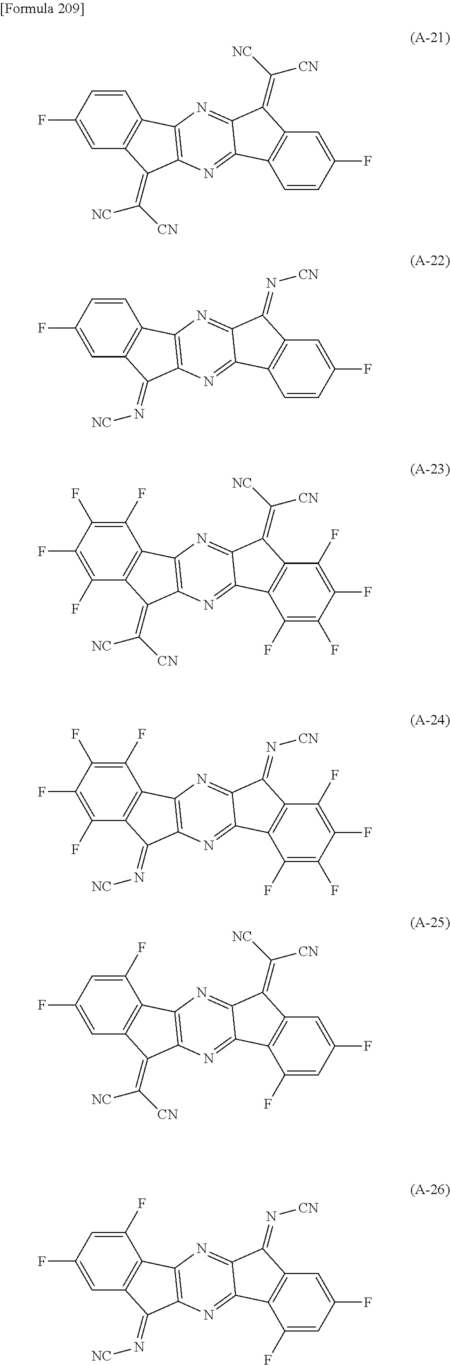 [SMILES: N#CC(C#N)=C1c2cc(F)ccc2-c2nc3c(nc21)-c1ccc(F)cc1C3=C(C#N)C#N ; N#C/N=c1\c2cc(F)ccc2c2nc3/c(=N/C#N)c4cc(F)ccc4c3nc12 ; N#CC(C#N)=C1c2nc3c(nc2-c2c(F)c(F)c(F)c(F)c21)C(=C(C#N)C#N)c1c(F)c(F)c(F)c(F)c1-3 ; N#C/N=c1/c2nc3c(nc2c2c(F)c(F)c(F)c(F)c12)/c(=N/C#N)c1c(F)c(F)c(F)c(F)c13 ; N#CC(C#N)=C1c2cc(F)cc(F)c2-c2nc3c(nc21)-c1c(F)cc(F)cc1C3=C(C#N)C#N ; N#C/N=c1\c2cc(F)cc(F)c2c2nc3/c(=N/C#N)c4cc(F)cc(F)c4c3nc12]

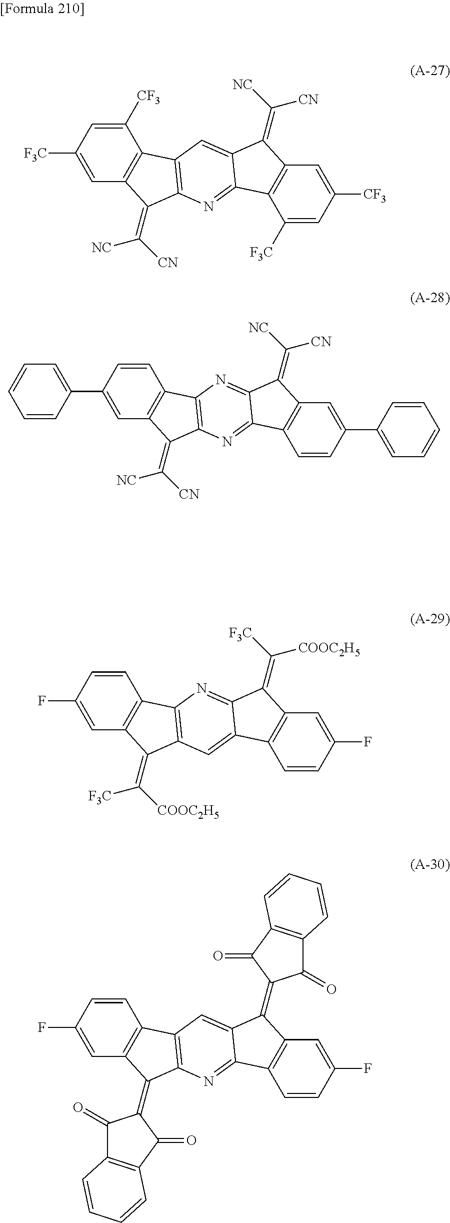

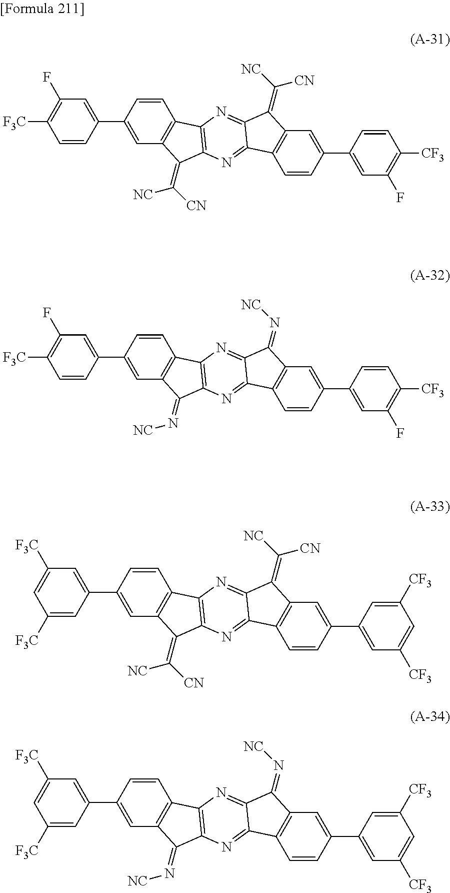 [SMILES: N#CC(C#N)=C1c2cc(-c3ccc(C(F)(F)F)c(F)c3)ccc2-c2nc3c(nc21)-c1ccc(-c2ccc(C(F)(F)F)c(F)c2)cc1C3=C(C#N)C#N ; N#C/N=c1/c2cc(-c3ccc(C(F)(F)F)c(F)c3)ccc2c2nc3/c(=N/C#N)c4cc(-c5ccc(C(F)(F)F)c(F)c5)ccc4c3nc12 ; N#CC(C#N)=C1c2cc(-c3cc(C(F)(F)F)cc(C(F)(F)F)c3)ccc2-c2nc3c(nc21)-c1ccc(-c2cc(C(F)(F)F)cc(C(F)(F)F)c2)cc1C3=C(C#N)C#N ; N#C/N=c1/c2cc(-c3cc(C(F)(F)F)cc(C(F)(F)F)c3)ccc2c2nc3/c(=N/C#N)c4cc(-c5cc(C(F)(F)F)cc(C(F)(F)F)c5)ccc4c3nc12]

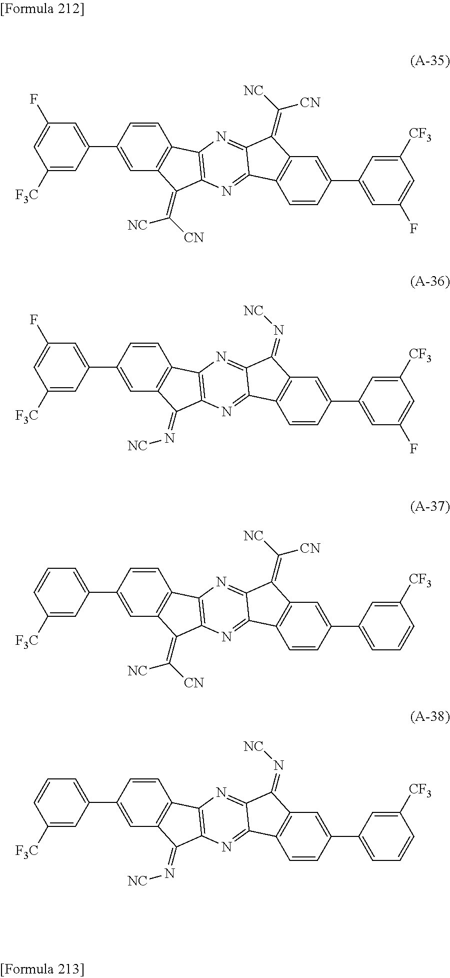 [SMILES: N#CC(C#N)=C1c2cc(-c3cc(F)cc(C(F)(F)F)c3)ccc2-c2nc3c(nc21)-c1ccc(-c2cc(F)cc(C(F)(F)F)c2)cc1C3=C(C#N)C#N ; N#C/N=c1/c2cc(-c3cc(F)cc(C(F)(F)F)c3)ccc2c2nc3/c(=N/C#N)c4cc(-c5cc(F)cc(C(F)(F)F)c5)ccc4c3nc12 ; N#CC(C#N)=C1c2cc(-c3cccc(C(F)(F)F)c3)ccc2-c2nc3c(nc21)-c1ccc(-c2cccc(C(F)(F)F)c2)cc1C3=C(C#N)C#N ; N#C/N=c1/c2cc(-c3cccc(C(F)(F)F)c3)ccc2c2nc3/c(=N/C#N)c4cc(-c5cccc(C(F)(F)F)c5)ccc4c3nc12]

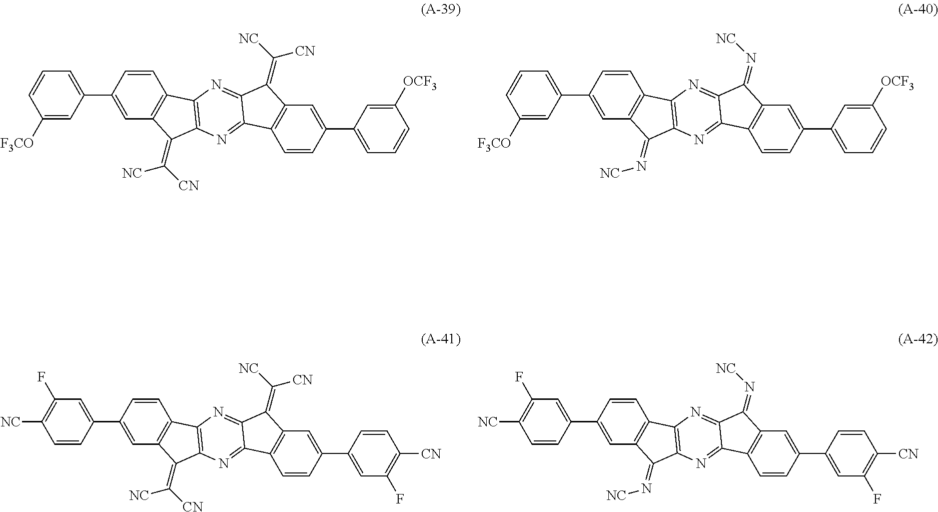 [SMILES: N#CC(C#N)=C1c2cc(-c3cccc(OC(F)(F)F)c3)ccc2-c2nc3c(nc21)-c1ccc(-c2cccc(OC(F)(F)F)c2)cc1C3=C(C#N)C#N ; N#C/N=c1/c2cc(-c3cccc(OC(F)(F)F)c3)ccc2c2nc3/c(=N/C#N)c4cc(-c5cccc(OC(F)(F)F)c5)ccc4c3nc12 ; N#CC(C#N)=C1c2cc(-c3ccc(C#N)c(F)c3)ccc2-c2nc3c(nc21)-c1ccc(-c2ccc(C#N)c(F)c2)cc1C3=C(C#N)C#N ; N#C/N=c1/c2cc(-c3ccc(C#N)c(F)c3)ccc2c2nc3/c(=N/C#N)c4cc(-c5ccc(C#N)c(F)c5)ccc4c3nc12]

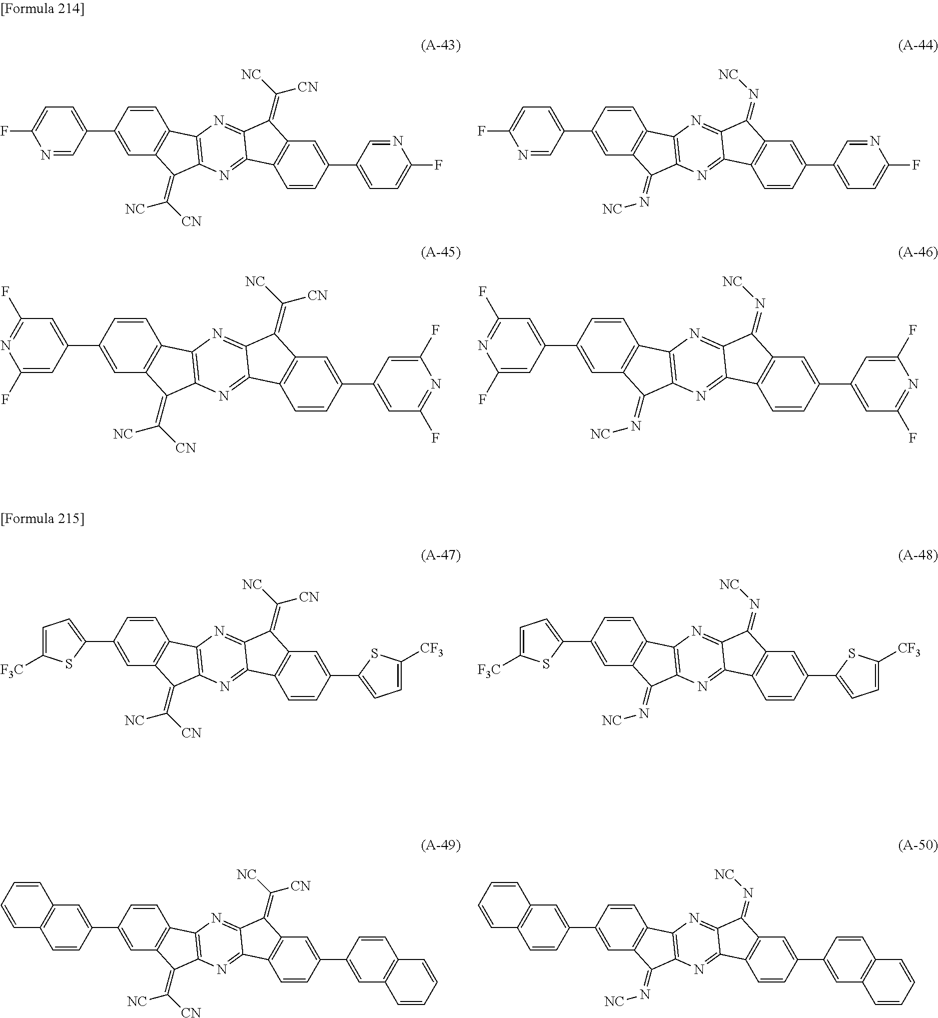

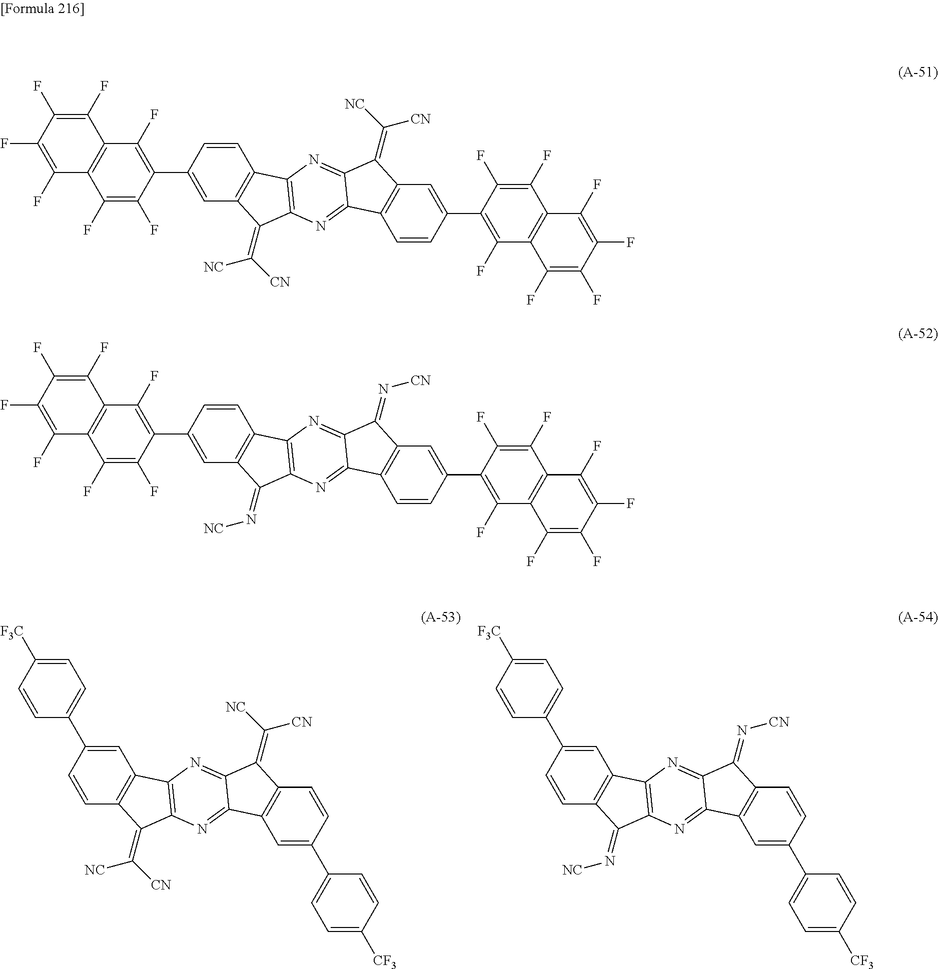 [SMILES: N#CC(C#N)=C1c2cc(-c3c(F)c(F)c4c(F)c(F)c(F)c(F)c4c3F)ccc2-c2nc3c(nc21)-c1ccc(-c2c(F)c(F)c4c(F)c(F)c(F)c(F)c4c2F)cc1C3=C(C#N)C#N ; N#C/N=c1\c2cc(-c3c(F)c(F)c4c(F)c(F)c(F)c(F)c4c3F)ccc2c2nc3/c(=N/C#N)c4cc(-c5c(F)c(F)c6c(F)c(F)c(F)c(F)c6c5F)ccc4c3nc12 ; N#CC(C#N)=C1c2ccc(-c3ccc(C(F)(F)F)cc3)cc2-c2nc3c(nc21)-c1cc(-c2ccc(C(F)(F)F)cc2)ccc1C3=C(C#N)C#N ; N#C/N=c1\c2ccc(-c3ccc(C(F)(F)F)cc3)cc2c2nc3/c(=N/C#N)c4ccc(-c5ccc(C(F)(F)F)cc5)cc4c3nc12]

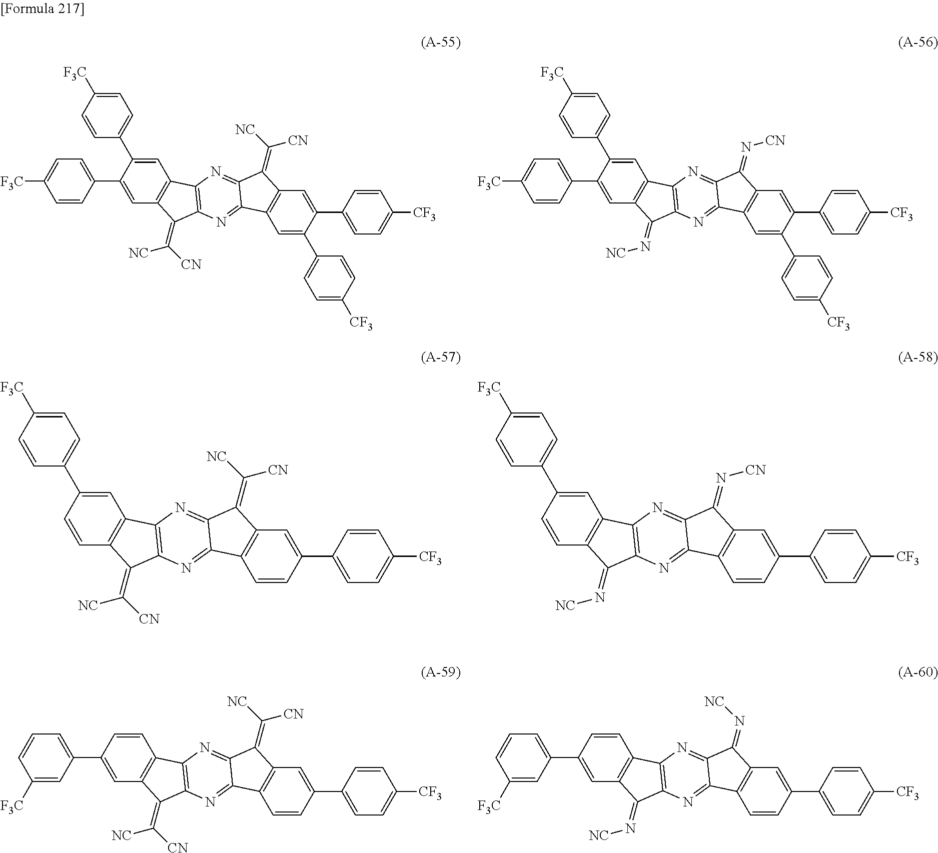

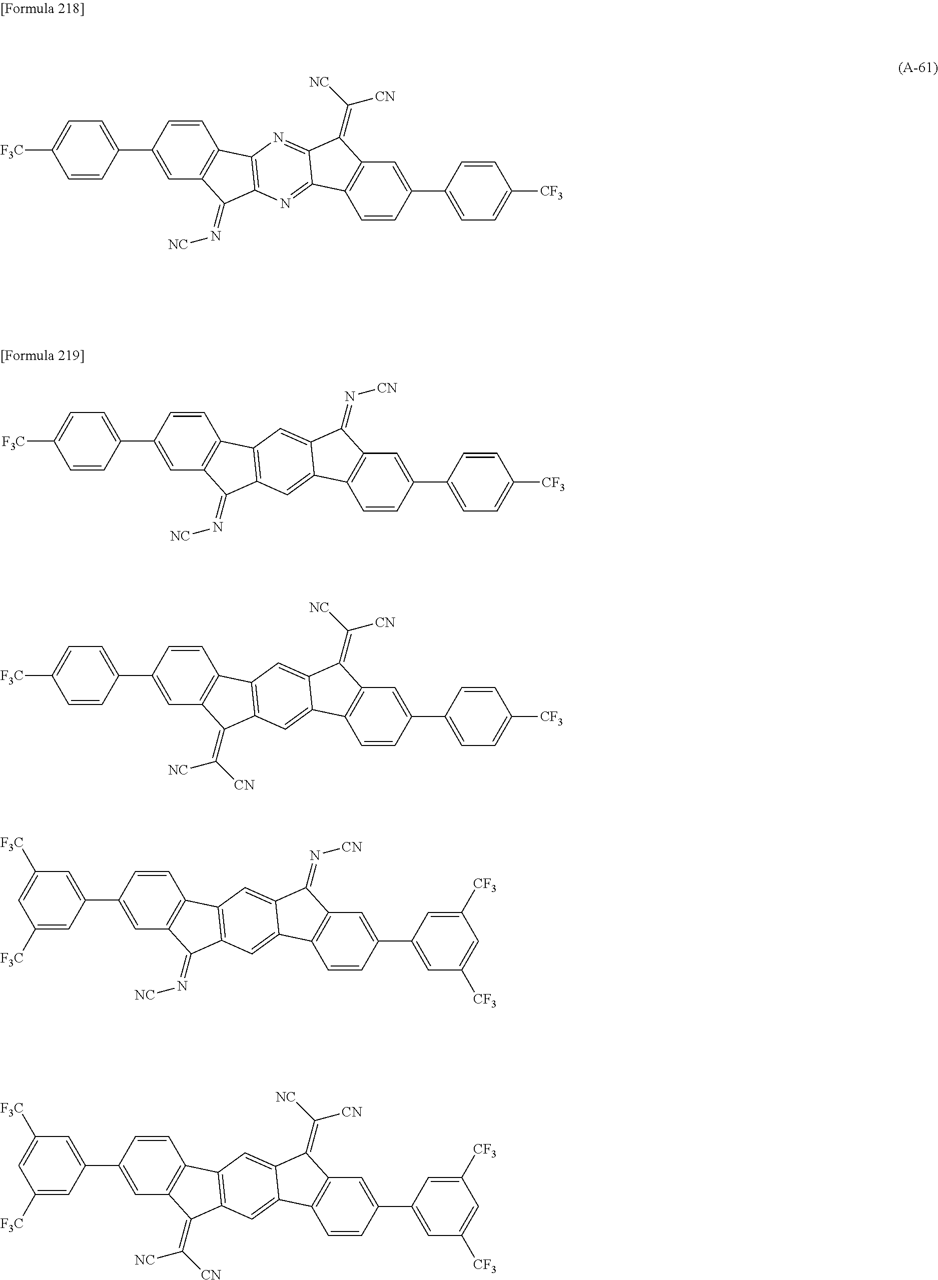

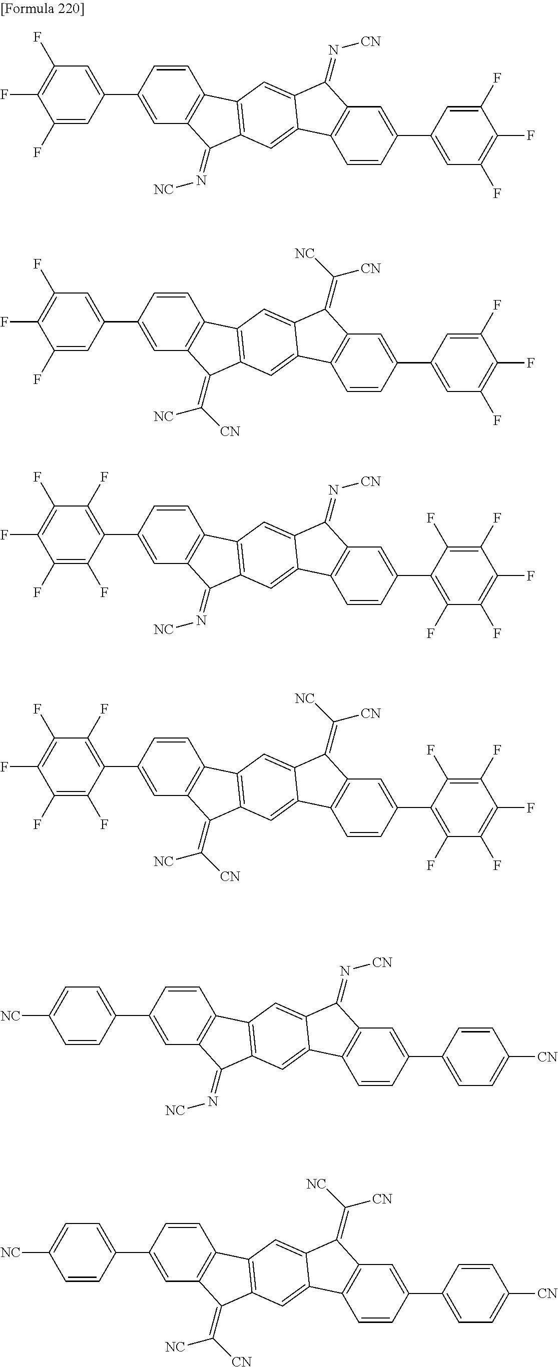

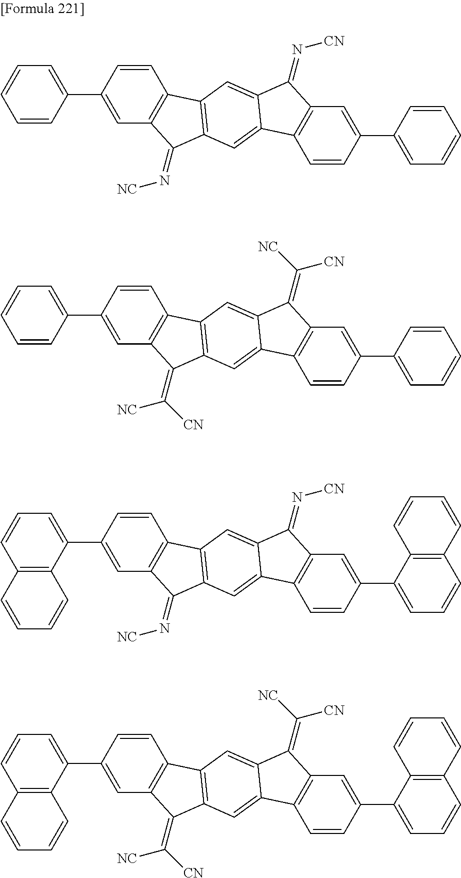 [SMILES: N#C/N=c1\c2cc(-c3ccccc3)ccc2c2cc3/c(=N/C#N)c4cc(-c5ccccc5)ccc4c3cc12 ; N#CC(C#N)=C1c2cc(-c3ccccc3)ccc2-c2cc3c(cc21)-c1ccc(-c2ccccc2)cc1C3=C(C#N)C#N ; N#C/N=c1\c2cc(-c3cccc4ccccc34)ccc2c2cc3/c(=N/C#N)c4cc(-c5cccc6ccccc56)ccc4c3cc12 ; N#CC(C#N)=C1c2cc(-c3cccc4ccccc34)ccc2-c2cc3c(cc21)-c1ccc(-c2cccc4ccccc24)cc1C3=C(C#N)C#N]

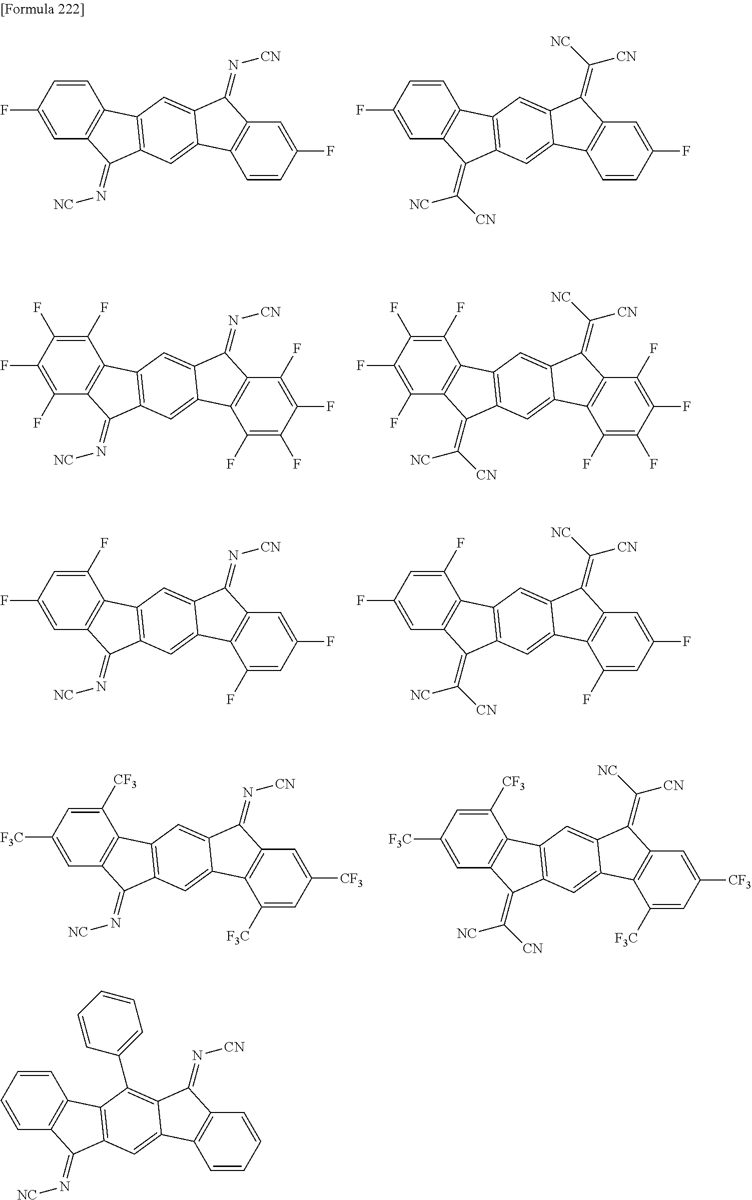

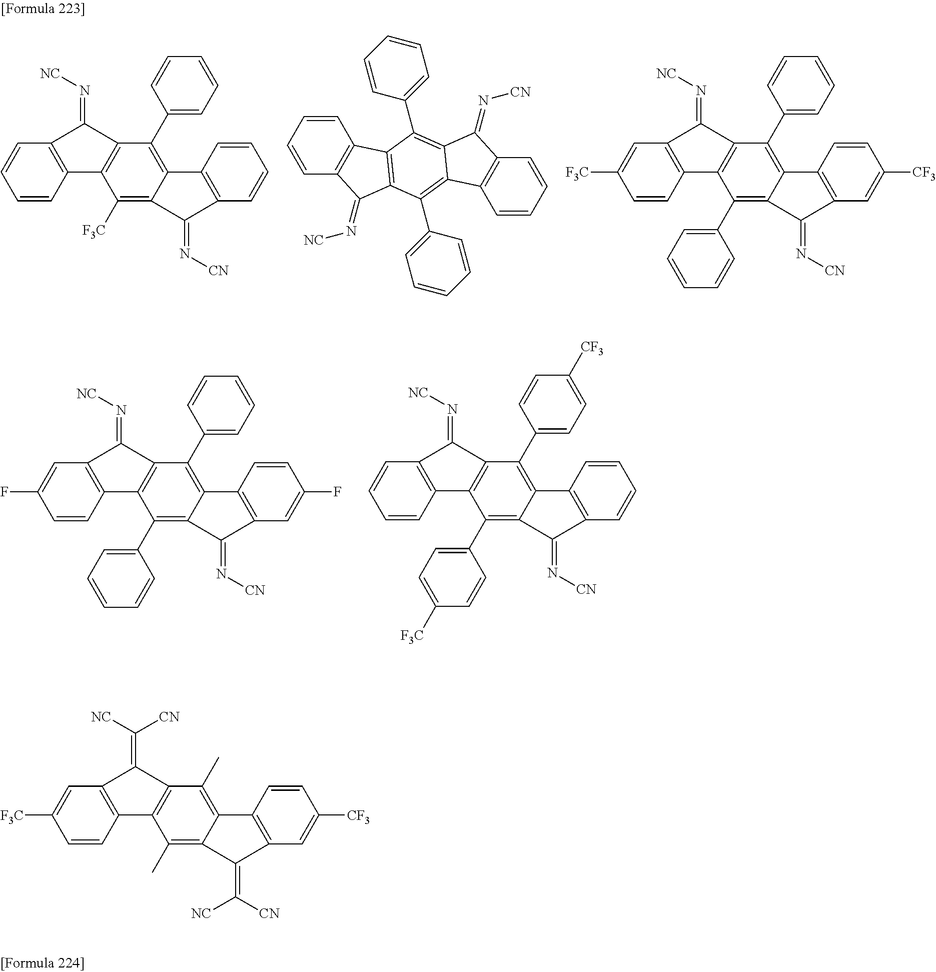

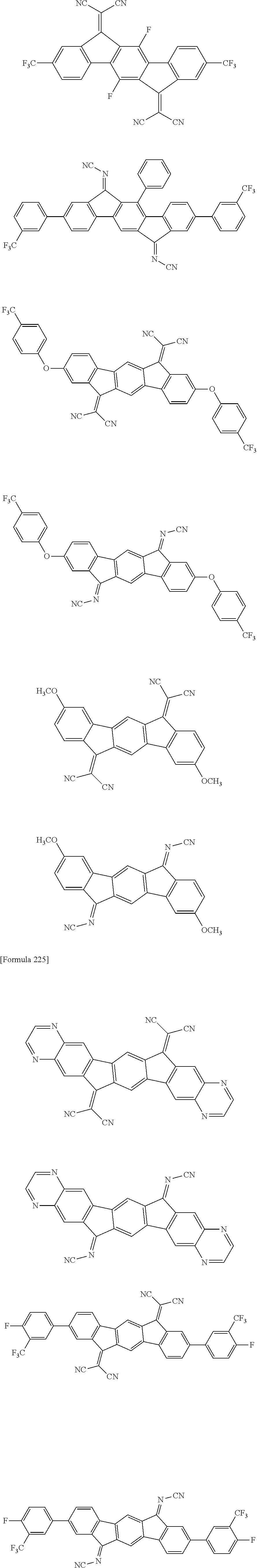

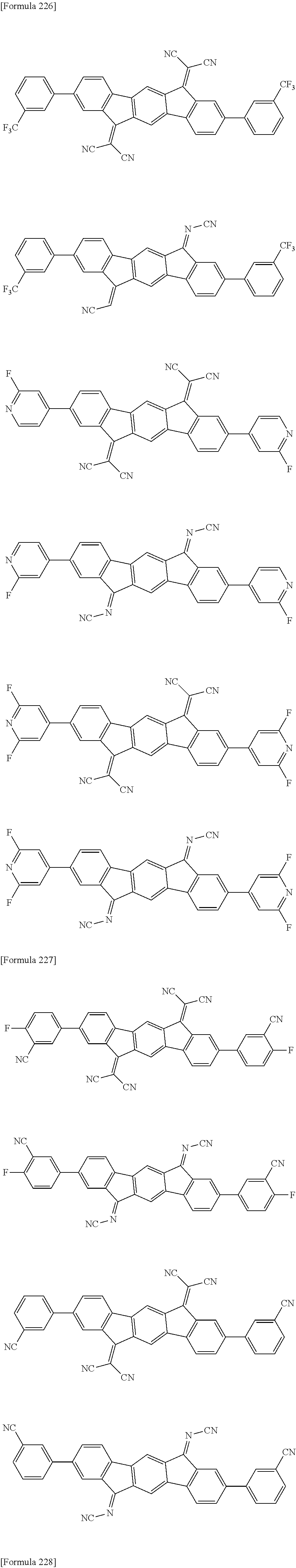

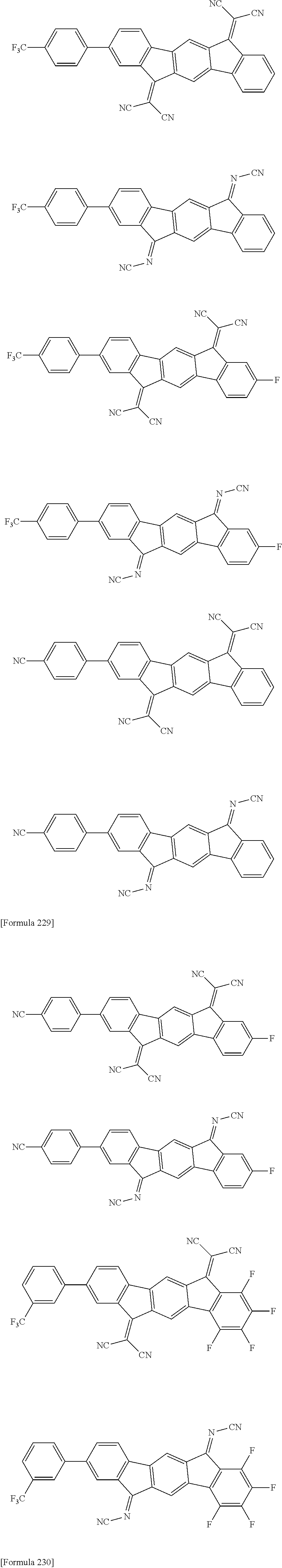

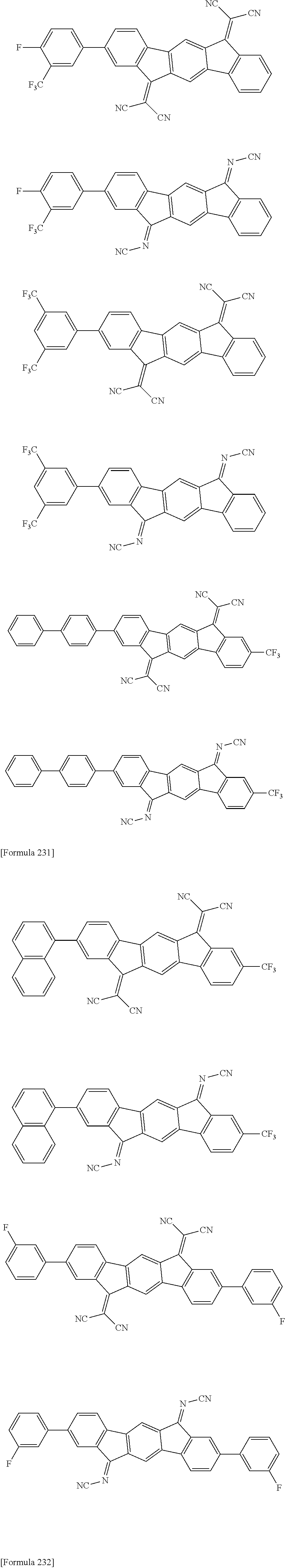

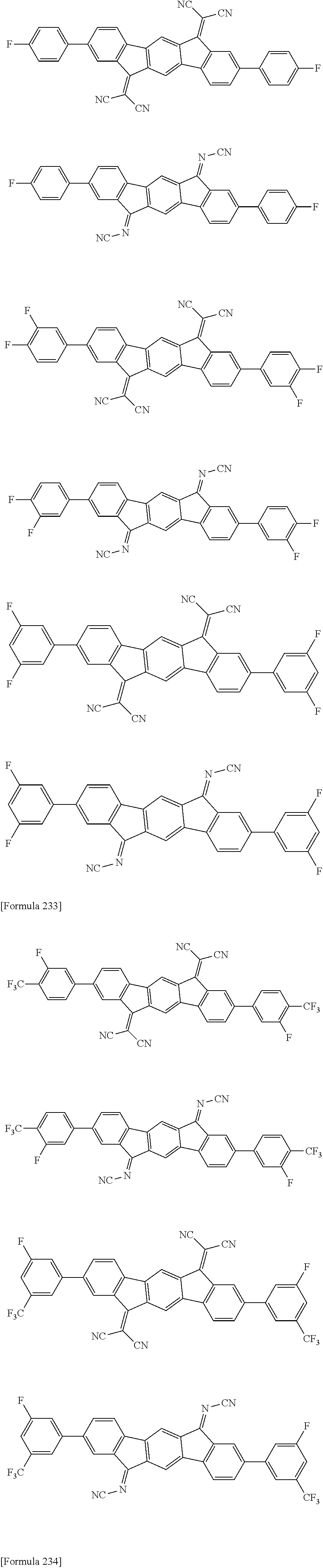

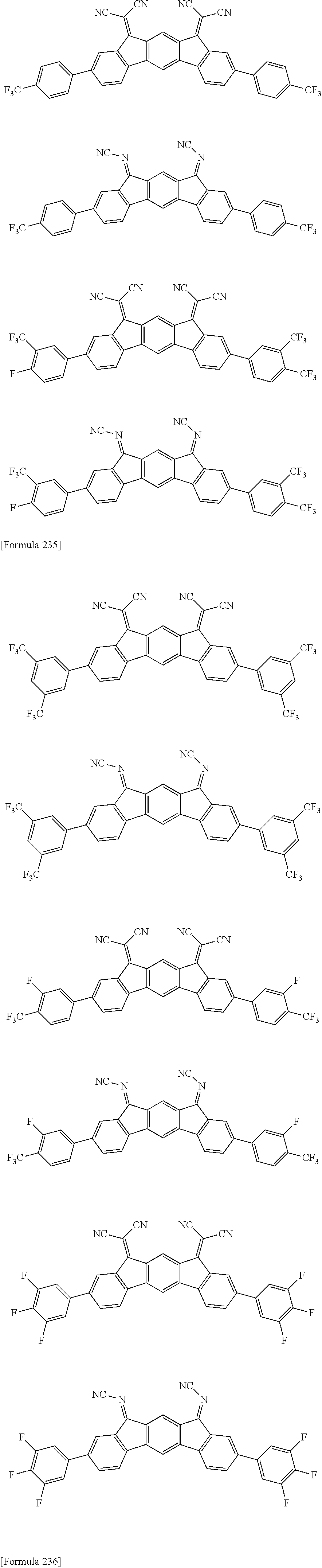

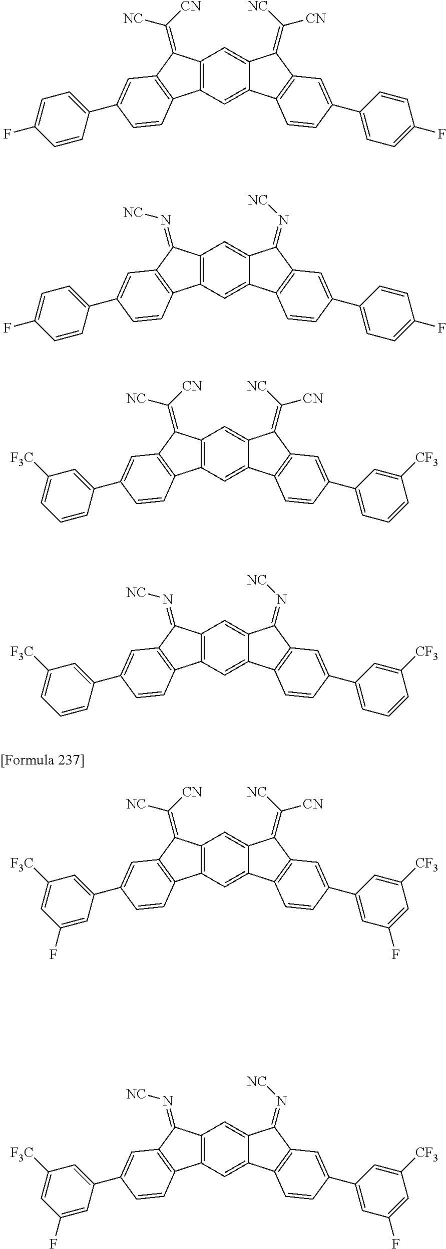

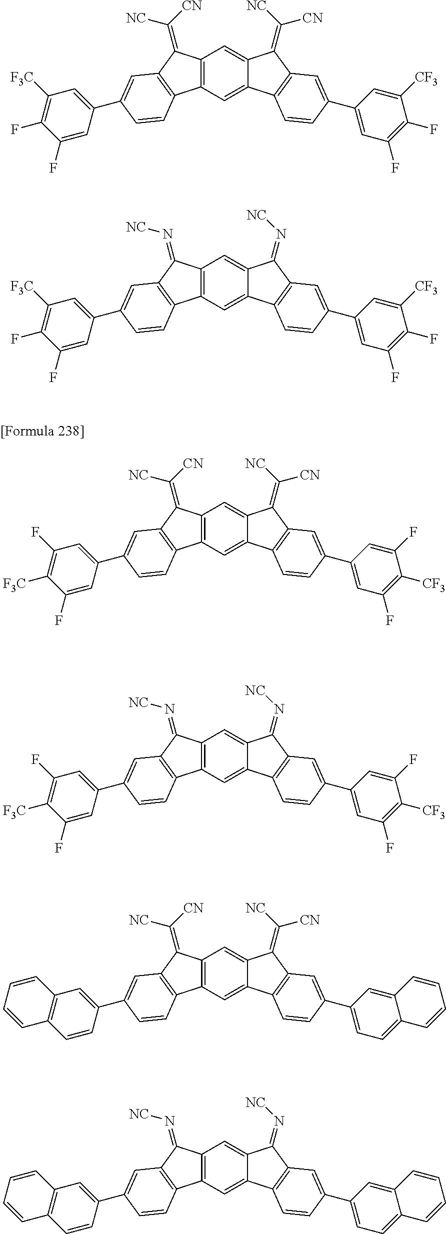

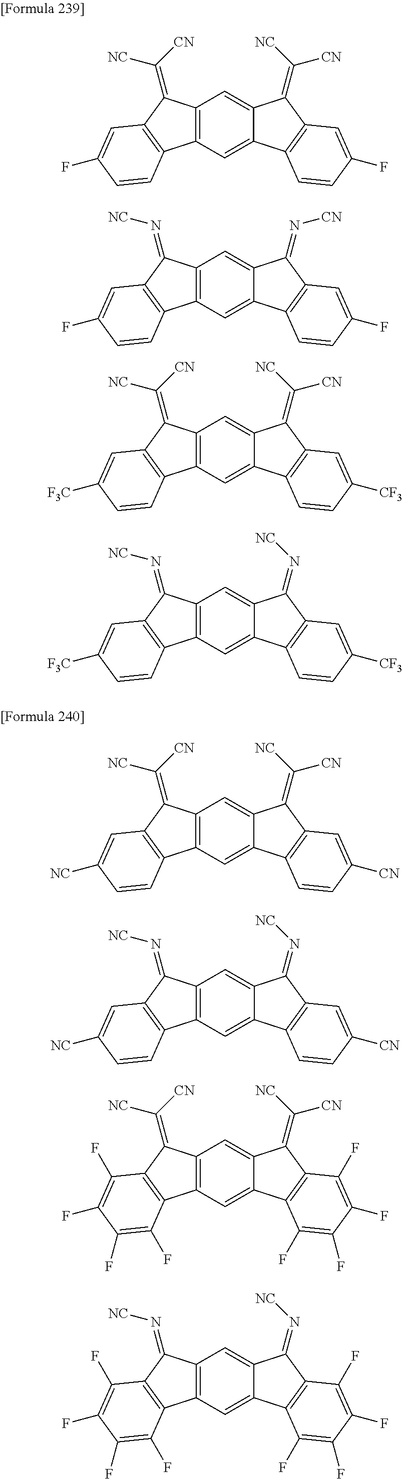

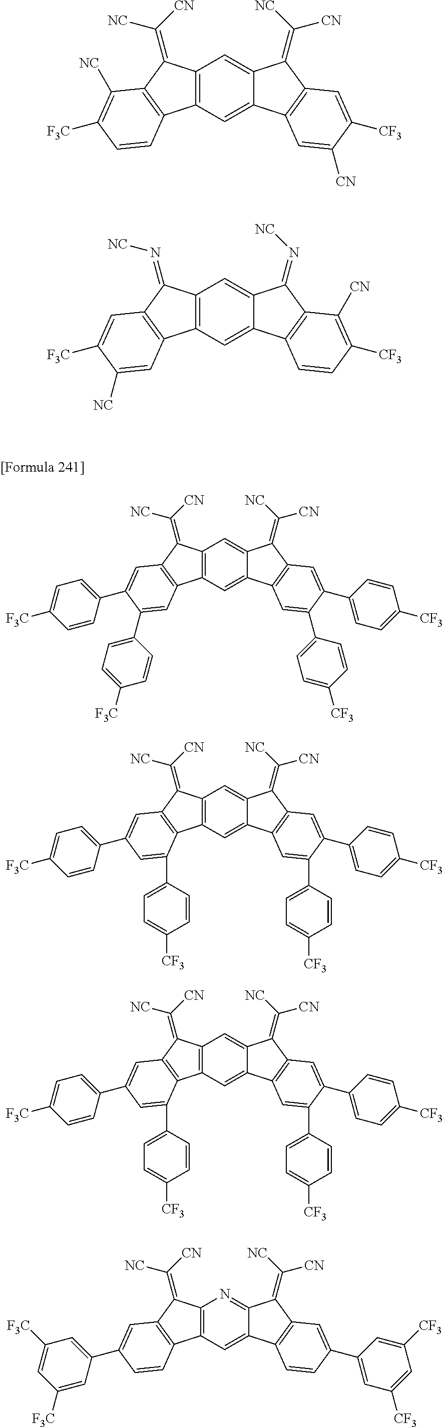

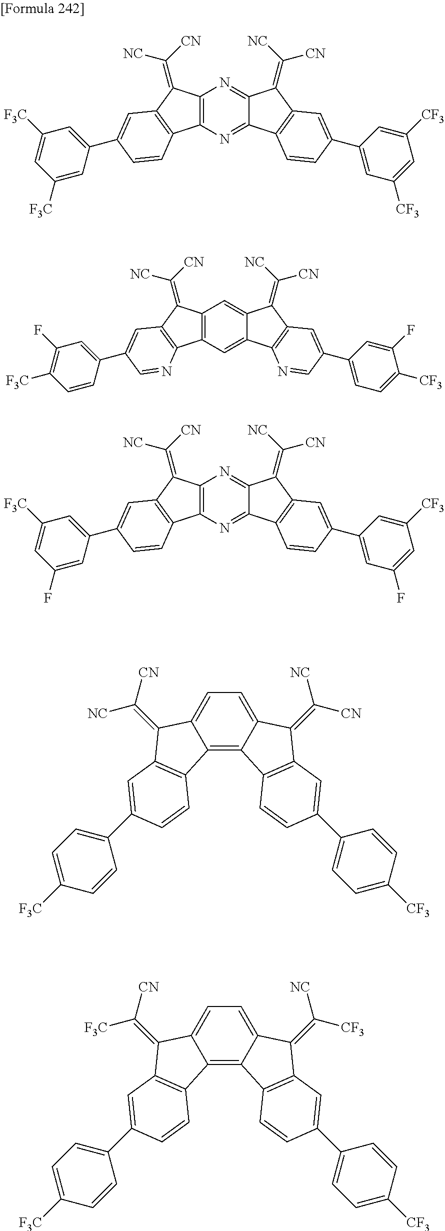

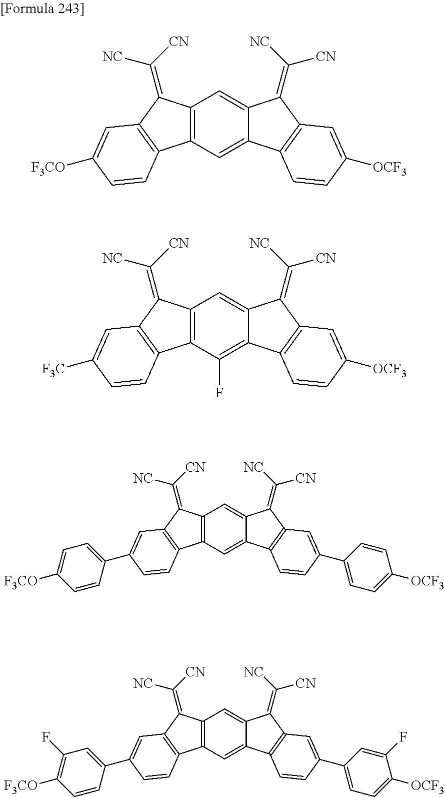 [SMILES: N#CC(C#N)=C1c2cc(OC(F)(F)F)ccc2-c2cc3c(cc21)C(=C(C#N)C#N)c1cc(OC(F)(F)F)ccc1-3 ; N#CC(C#N)=C1c2cc(OC(F)(F)F)ccc2-c2c1cc1c(c2F)-c2ccc(C(F)(F)F)cc2C1=C(C#N)C#N ; N#CC(C#N)=C1c2cc(-c3ccc(OC(F)(F)F)cc3)ccc2-c2cc3c(cc21)C(=C(C#N)C#N)c1cc(-c2ccc(OC(F)(F)F)cc2)ccc1-3 ; N#CC(C#N)=C1c2cc(-c3ccc(OC(F)(F)F)c(F)c3)ccc2-c2cc3c(cc21)C(=C(C#N)C#N)c1cc(-c2ccc(OC(F)(F)F)c(F)c2)ccc1-3]

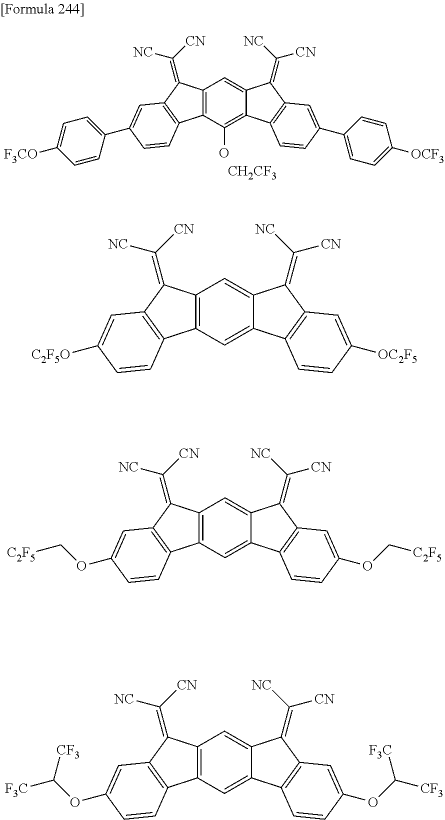

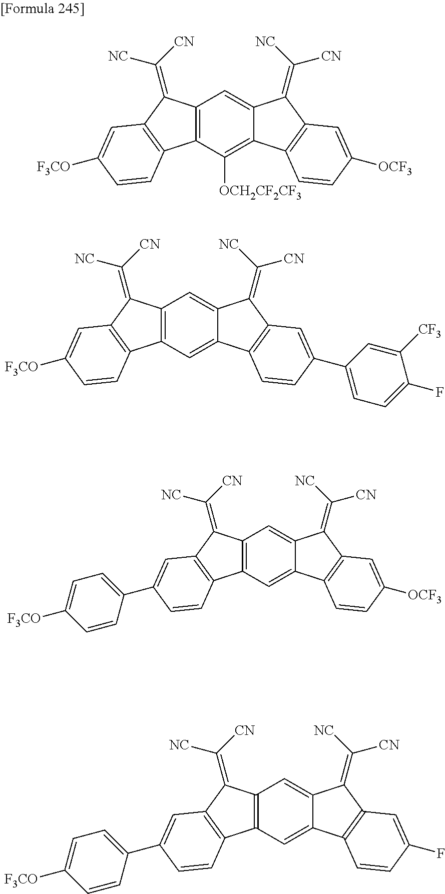

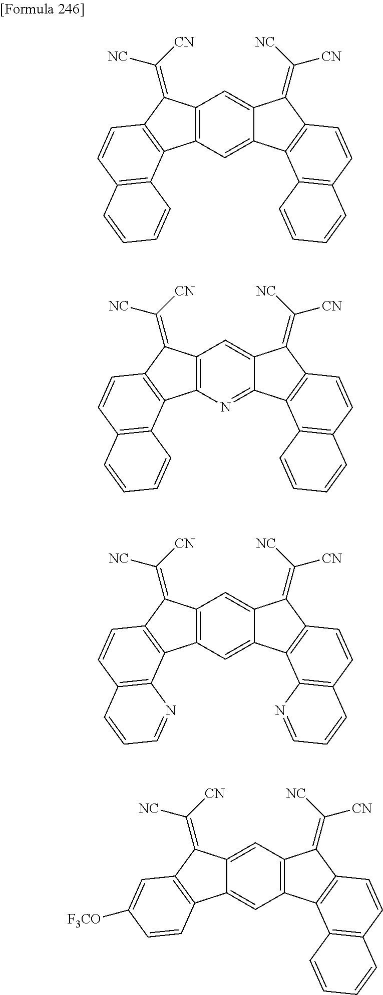 [SMILES: N#CC(C#N)=C1c2cc3c(cc2-c2c1ccc1ccccc21)-c1c(ccc2ccccc12)C3=C(C#N)C#N ; N#CC(C#N)=C1c2cc3c(nc2-c2c1ccc1ccccc21)-c1c(ccc2ccccc12)C3=C(C#N)C#N ; N#CC(C#N)=C1c2cc3c(cc2-c2c1ccc1cccnc21)-c1c(ccc2cccnc12)C3=C(C#N)C#N ; N#CC(C#N)=C1c2cc(OC(F)(F)F)ccc2-c2cc3c(cc21)C(=C(C#N)C#N)c1ccc2ccccc2c1-3]

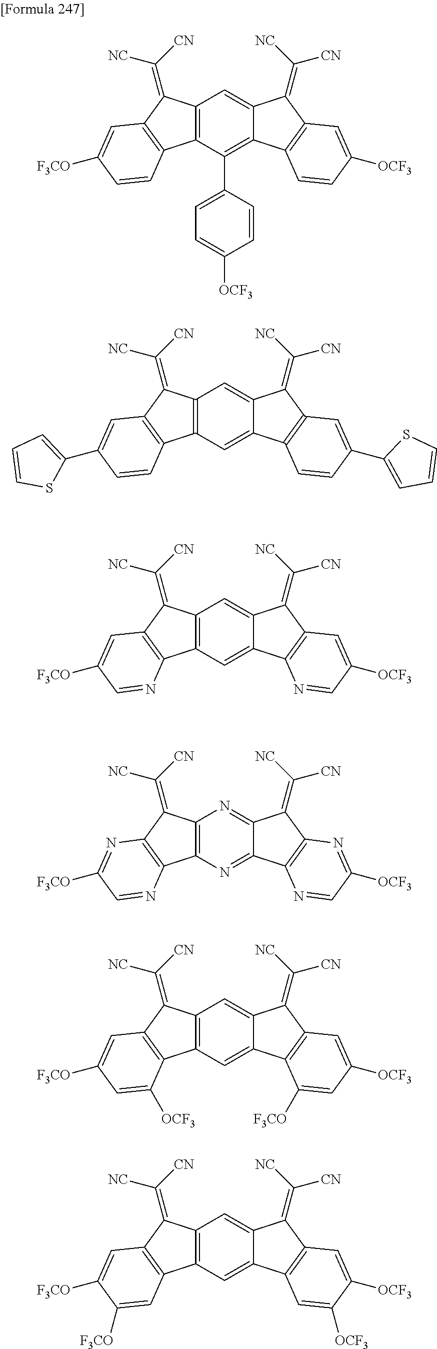

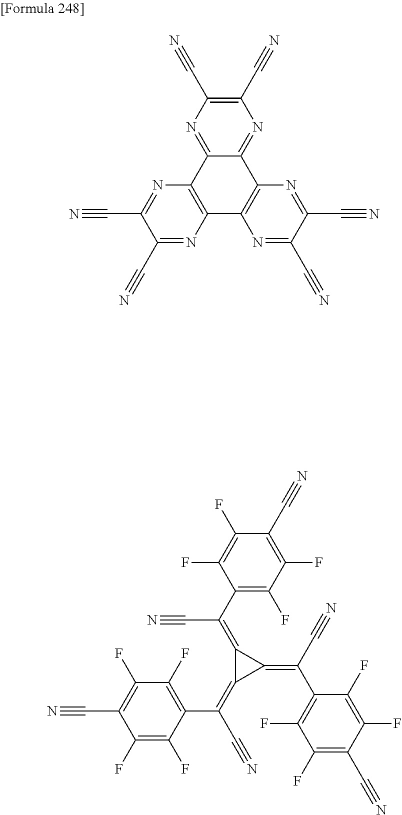 [SMILES: N#Cc1nc2c3nc(C#N)c(C#N)nc3c3nc(C#N)c(C#N)nc3c2nc1C#N ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

### Additive Material

The fourth material as the additive material in an exemplary arrangement of the organic EL devices of the first, second, and third exemplary embodiments as well as the fourth material and the sixth material as the additive material in an exemplary arrangement of the organic EL devices of the fourth, fifth, and sixth exemplary embodiments are preferably each independently at least one compound selected from compounds listed below.

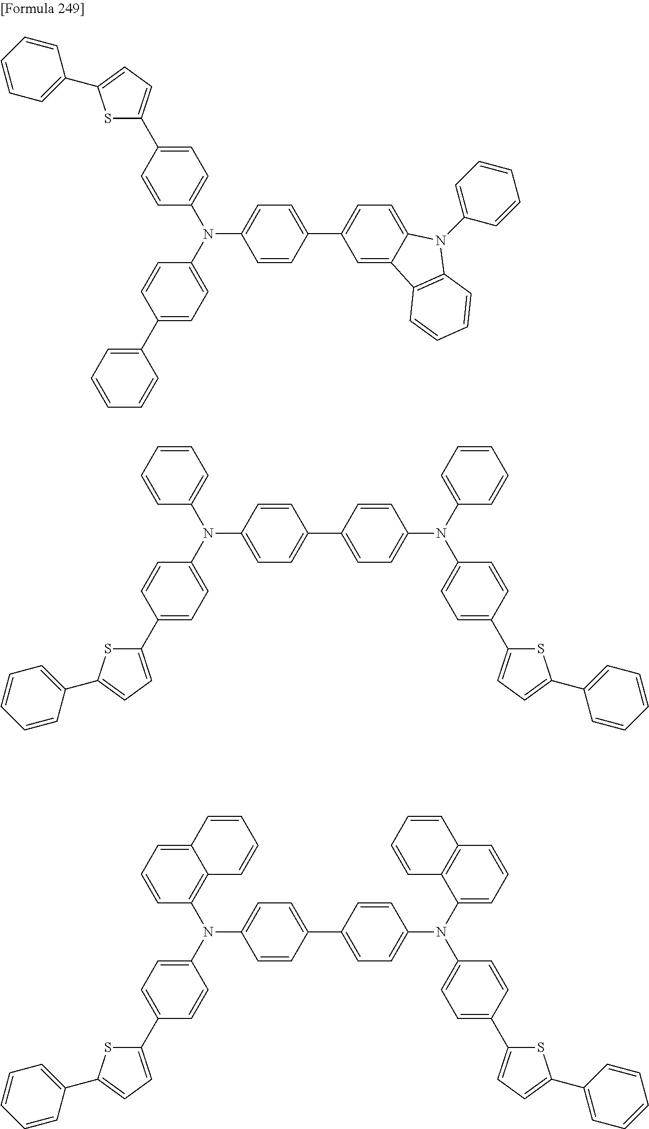

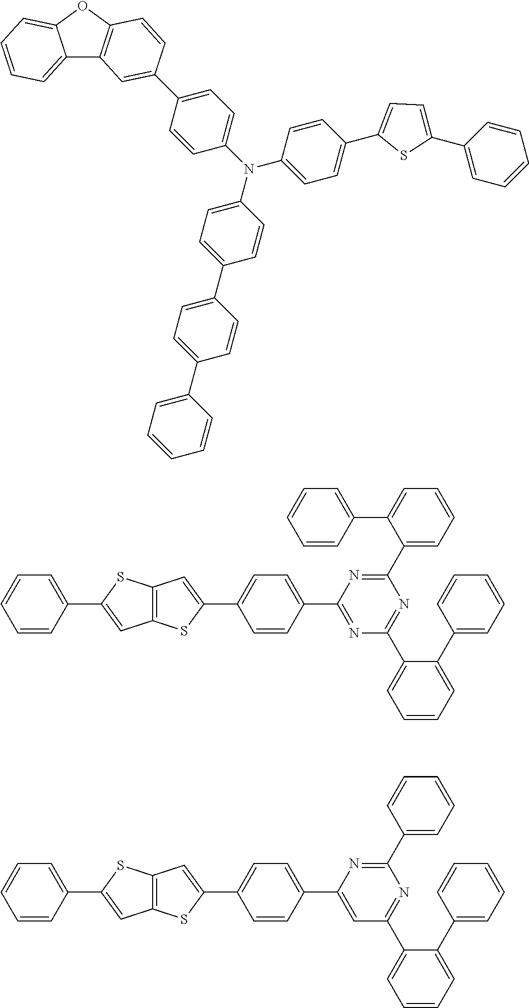 [SMILES: c1ccc(-c2ccc(-c3ccc(N(c4ccc(-c5ccc6oc7ccccc7c6c5)cc4)c4ccc(-c5ccc(-c6ccccc6)s5)cc4)cc3)cc2)cc1 ; c1ccc(-c2cc3sc(-c4ccc(-c5nc(-c6ccccc6-c6ccccc6)nc(-c6ccccc6-c6ccccc6)n5)cc4)cc3s2)cc1 ; c1ccc(-c2nc(-c3ccc(-c4cc5sc(-c6ccccc6)cc5s4)cc3)cc(-c3ccccc3-c3ccccc3)n2)cc1]

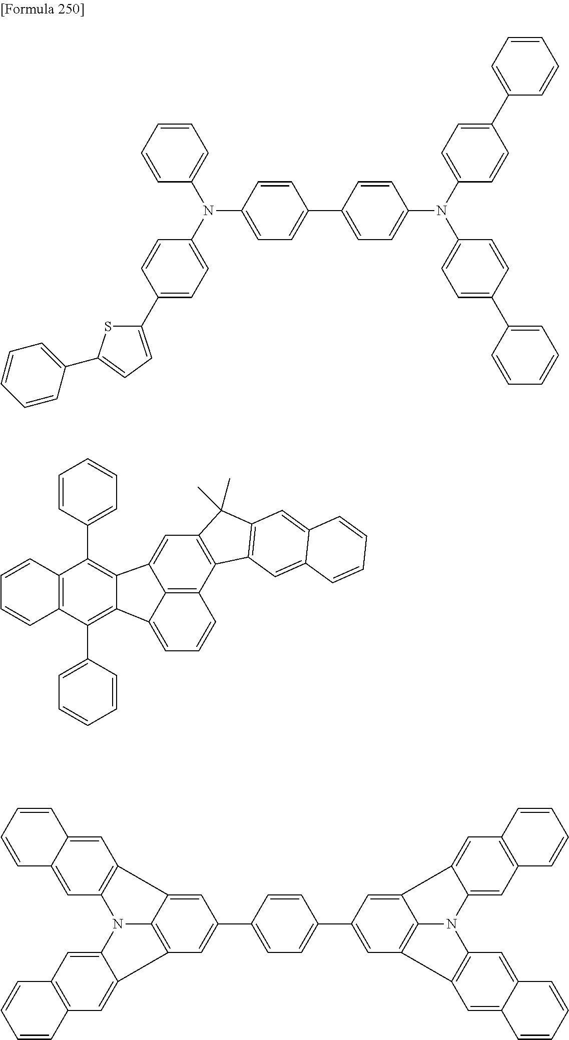

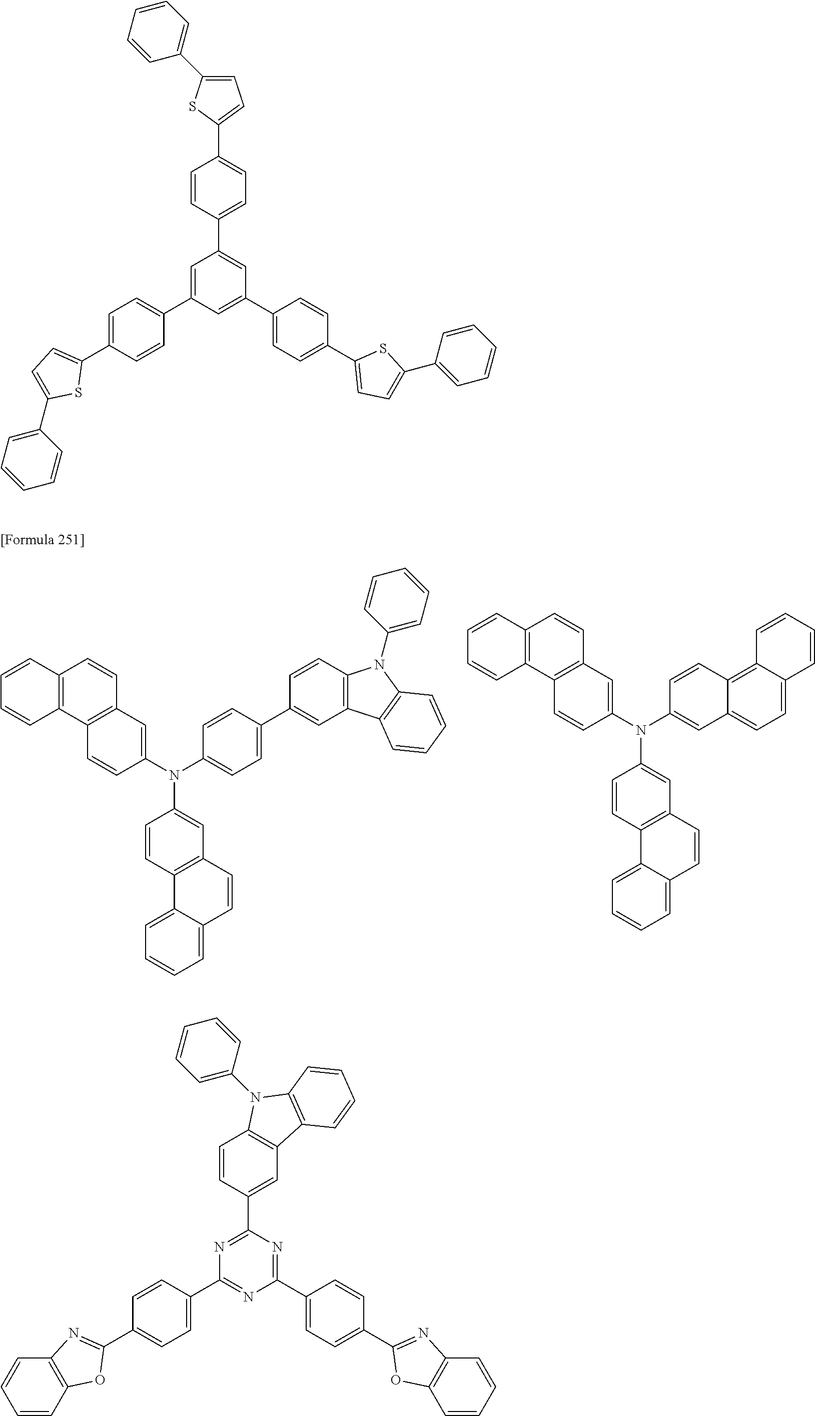 [SMILES: c1ccc(-c2ccc(-c3ccc(-c4cc(-c5ccc(-c6ccc(-c7ccccc7)s6)cc5)cc(-c5ccc(-c6ccc(-c7ccccc7)s6)cc5)c4)cc3)s2)cc1 ; c1ccc(-n2c3ccccc3c3cc(-c4ccc(N(c5ccc6c(ccc7ccccc76)c5)c5ccc6c(ccc7ccccc76)c5)cc4)ccc32)cc1 ; c1ccc2c(c1)ccc1cc(N(c3ccc4c(ccc5ccccc54)c3)c3ccc4c(ccc5ccccc54)c3)ccc12 ; c1ccc(-n2c3ccccc3c3cc(-c4nc(-c5ccc(-c6nc7ccccc7o6)cc5)nc(-c5ccc(-c6nc7ccccc7o6)cc5)n4)ccc32)cc1]

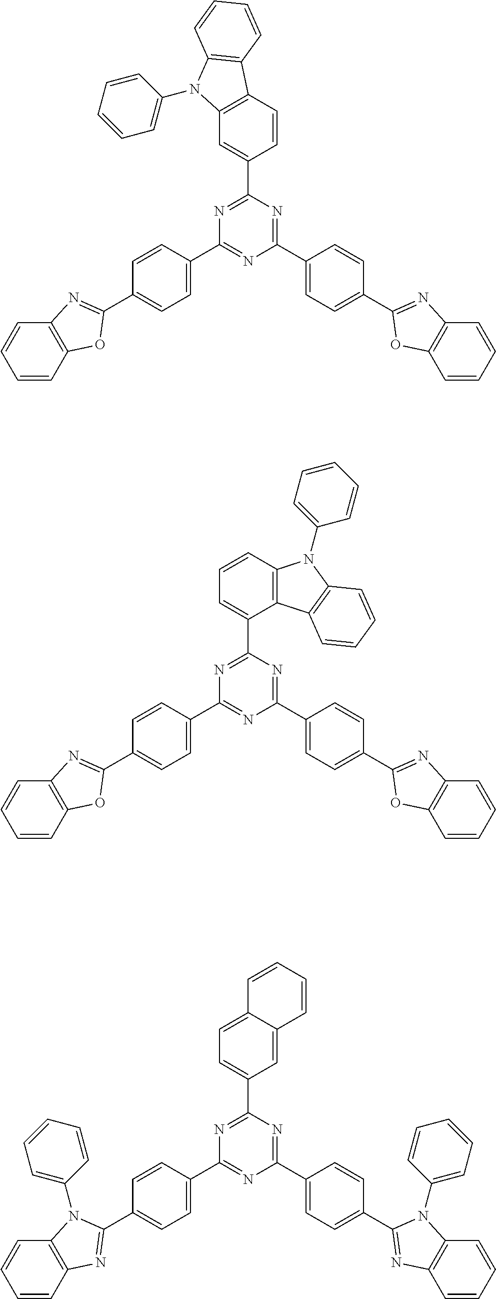 [SMILES: c1ccc(-n2c3ccccc3c3ccc(-c4nc(-c5ccc(-c6nc7ccccc7o6)cc5)nc(-c5ccc(-c6nc7ccccc7o6)cc5)n4)cc32)cc1 ; c1ccc(-n2c3ccccc3c3c(-c4nc(-c5ccc(-c6nc7ccccc7o6)cc5)nc(-c5ccc(-c6nc7ccccc7o6)cc5)n4)cccc32)cc1 ; c1ccc(-n2c(-c3ccc(-c4nc(-c5ccc(-c6nc7ccccc7n6-c6ccccc6)cc5)nc(-c5ccc6ccccc6c5)n4)cc3)nc3ccccc32)cc1]

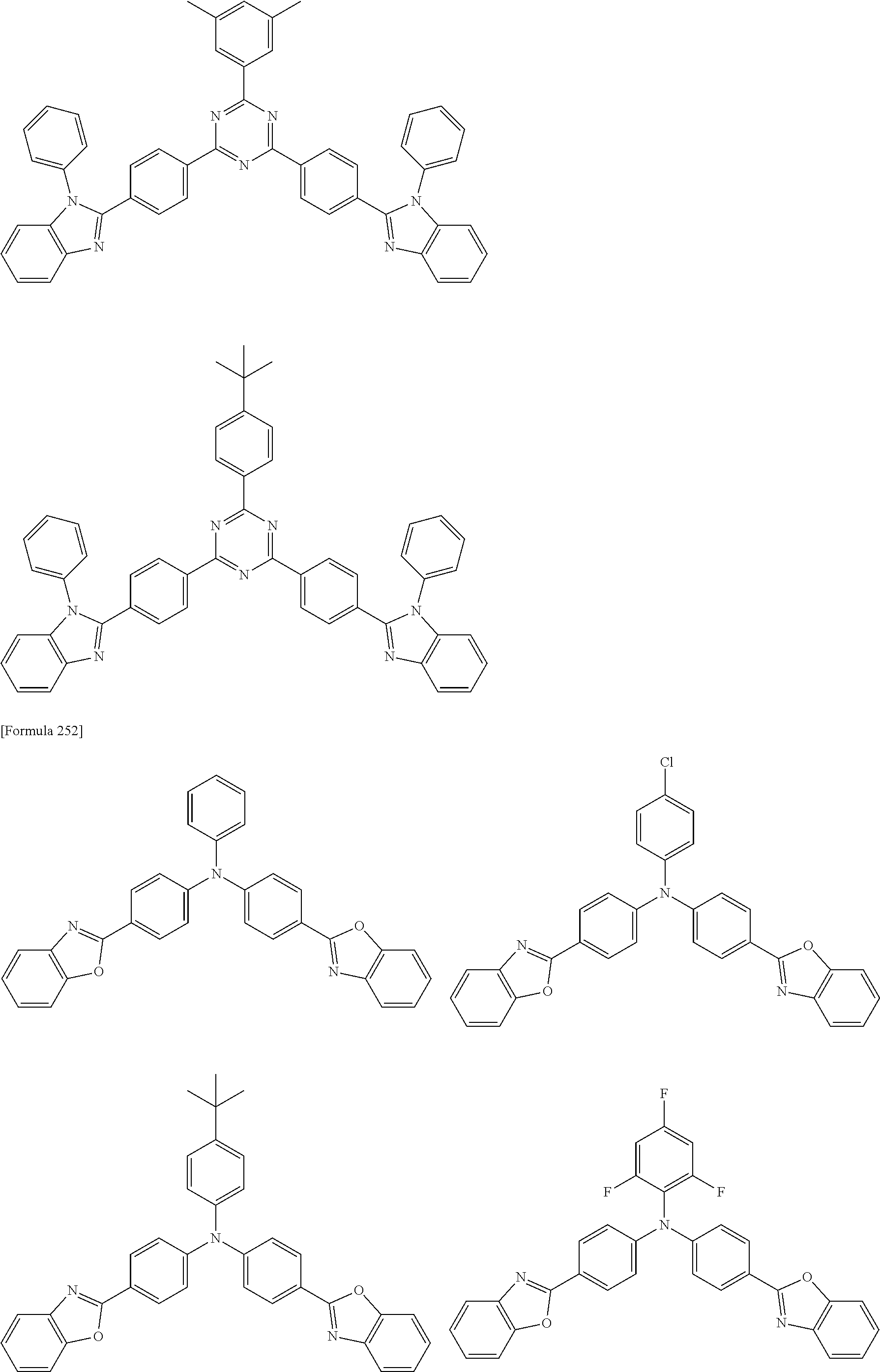

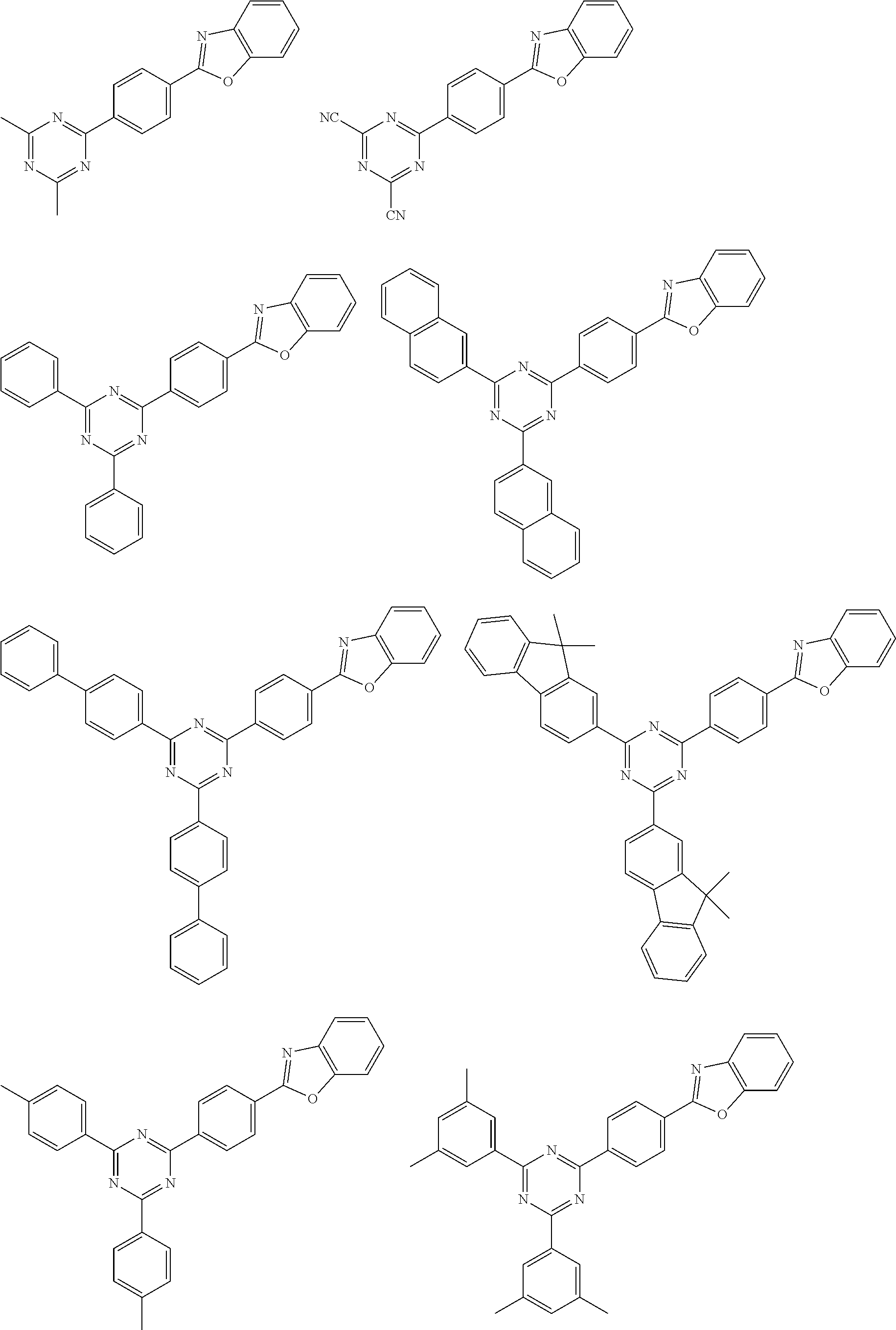

The fifth material as the additive material in an exemplary arrangement of the organic EL devices of the first, second, and third exemplary embodiments as well as the fifth material as the additive material in an exemplary arrangement of the organic EL devices of the fourth, fifth, and sixth exemplary embodiments are preferably each independently at least one compound selected from compounds listed below.

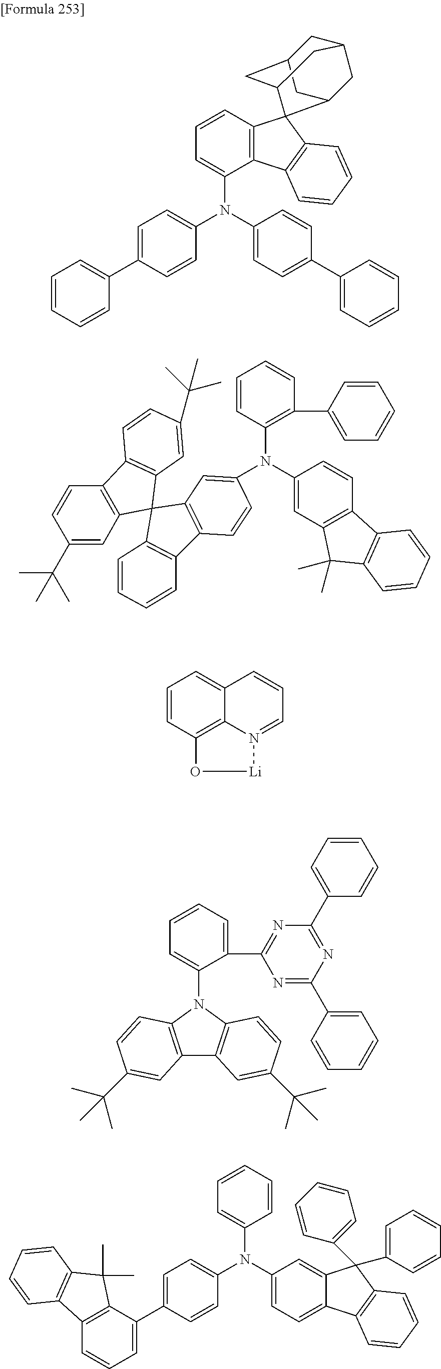

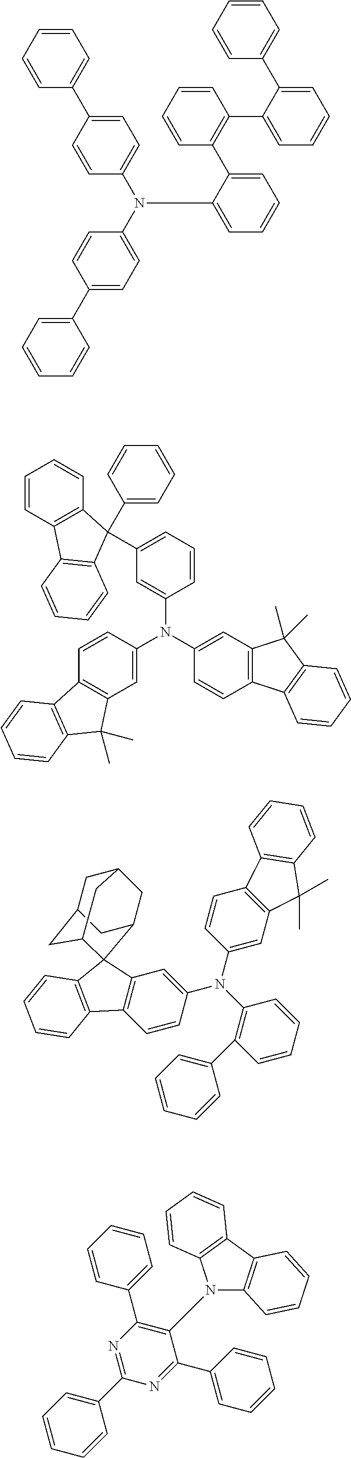 [SMILES: c1ccc(-c2ccc(N(c3ccc(-c4ccccc4)cc3)c3ccccc3-c3ccccc3-c3ccccc3-c3ccccc3)cc2)cc1 ; CC1(C)c2ccccc2-c2ccc(N(c3cccc(C4(c5ccccc5)c5ccccc5-c5ccccc54)c3)c3ccc4c(c3)C(C)(C)c3ccccc3-4)cc21 ; CC1(C)c2ccccc2-c2ccc(N(c3ccc4c(c3)C3(c5ccccc5-4)C4CC5CC(C4)CC3C5)c3ccccc3-c3ccccc3)cc21 ; c1ccc(-c2nc(-c3ccccc3)c(-n3c4ccccc4c4ccccc43)c(-c3ccccc3)n2)cc1]

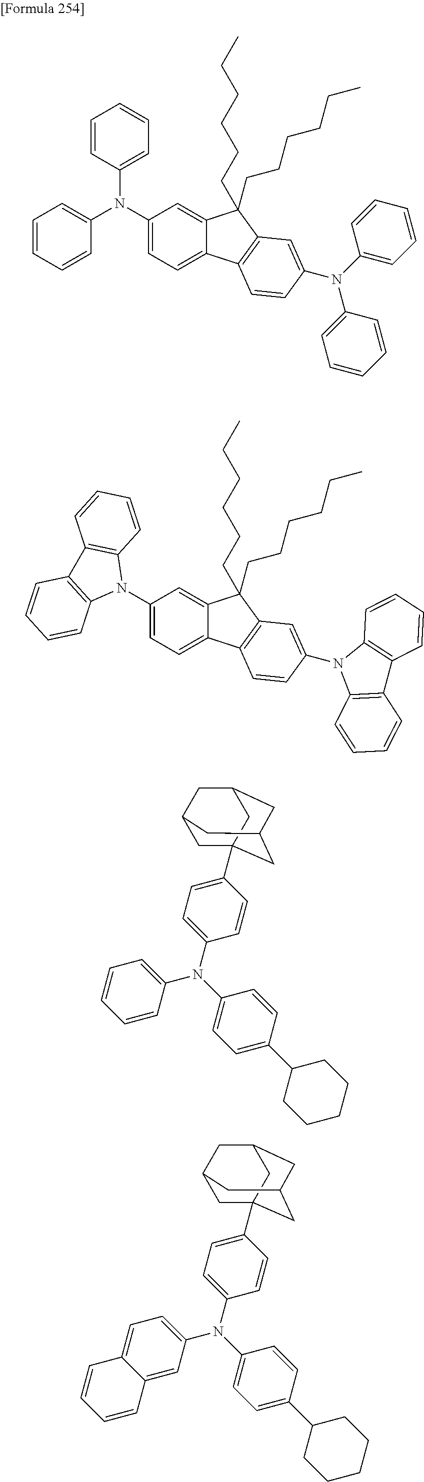

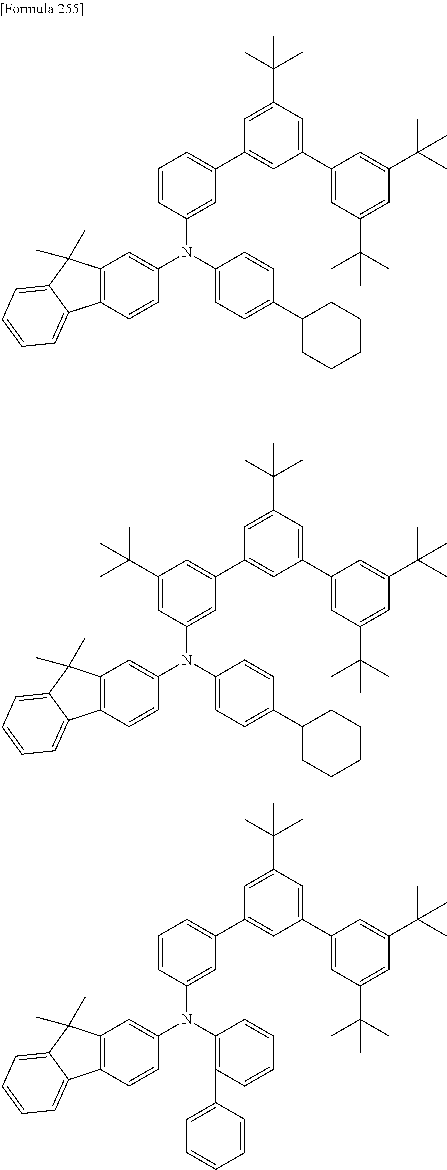 [SMILES: CC(C)(C)c1cc(-c2cccc(N(c3ccc(C4CCCCC4)cc3)c3ccc4c(c3)C(C)(C)c3ccccc3-4)c2)cc(-c2cc(C(C)(C)C)cc(C(C)(C)C)c2)c1 ; CC(C)(C)c1cc(-c2cc(N(c3ccc(C4CCCCC4)cc3)c3ccc4c(c3)C(C)(C)c3ccccc3-4)cc(C(C)(C)C)c2)cc(-c2cc(C(C)(C)C)cc(C(C)(C)C)c2)c1 ; CC(C)(C)c1cc(-c2cccc(N(c3ccc4c(c3)C(C)(C)c3ccccc3-4)c3ccccc3-c3ccccc3)c2)cc(-c2cc(C(C)(C)C)cc(C(C)(C)C)c2)c1]

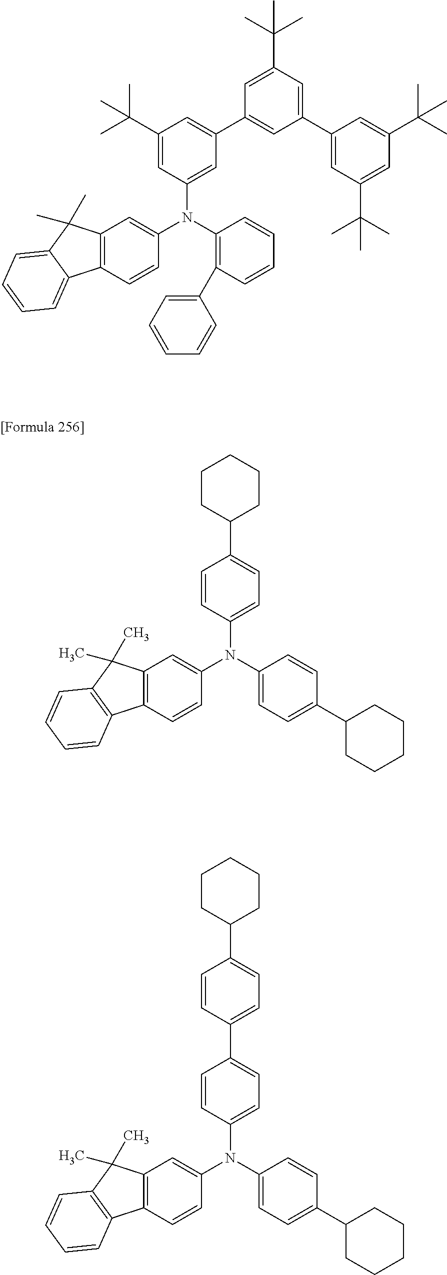 [SMILES: CC(C)(C)c1cc(-c2cc(N(c3ccc4c(c3)C(C)(C)c3ccccc3-4)c3ccccc3-c3ccccc3)cc(C(C)(C)C)c2)cc(-c2cc(C(C)(C)C)cc(C(C)(C)C)c2)c1 ; CC1(C)c2ccccc2-c2ccc(N(c3ccc(C4CCCCC4)cc3)c3ccc(C4CCCCC4)cc3)cc21 ; CC1(C)c2ccccc2-c2ccc(N(c3ccc(-c4ccc(C5CCCCC5)cc4)cc3)c3ccc(C4CCCCC4)cc3)cc21]

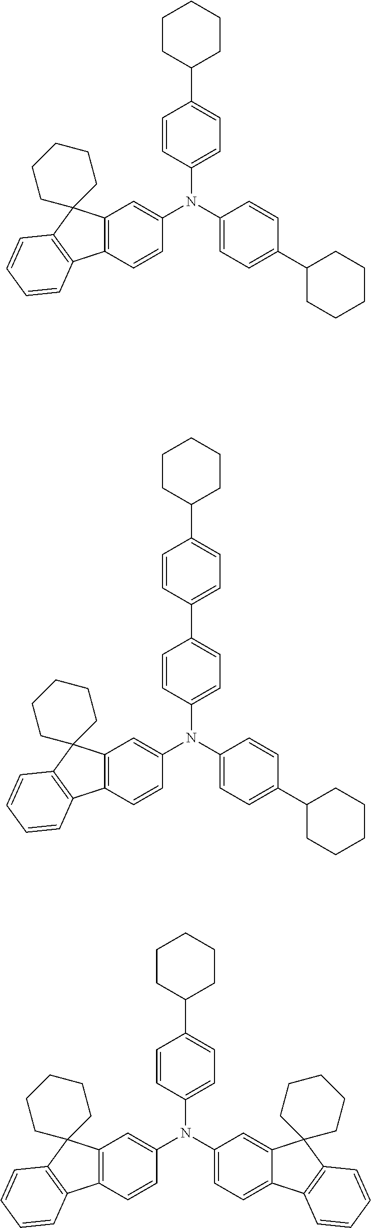 [SMILES: c1ccc2c(c1)-c1ccc(N(c3ccc(C4CCCCC4)cc3)c3ccc(C4CCCCC4)cc3)cc1C21CCCCC1 ; c1ccc2c(c1)-c1ccc(N(c3ccc(-c4ccc(C5CCCCC5)cc4)cc3)c3ccc(C4CCCCC4)cc3)cc1C21CCCCC1 ; c1ccc2c(c1)-c1ccc(N(c3ccc(C4CCCCC4)cc3)c3ccc4c(c3)C3(CCCCC3)c3ccccc3-4)cc1C21CCCCC1]

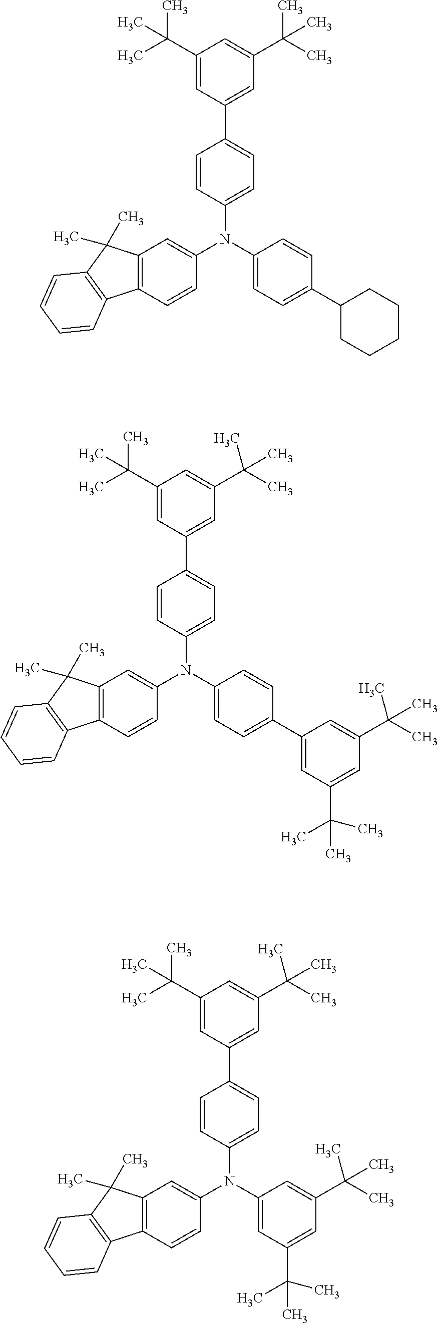 [SMILES: CC(C)(C)c1cc(-c2ccc(N(c3ccc(C4CCCCC4)cc3)c3ccc4c(c3)C(C)(C)c3ccccc3-4)cc2)cc(C(C)(C)C)c1 ; CC(C)(C)c1cc(-c2ccc(N(c3ccc(-c4cc(C(C)(C)C)cc(C(C)(C)C)c4)cc3)c3ccc4c(c3)C(C)(C)c3ccccc3-4)cc2)cc(C(C)(C)C)c1 ; CC(C)(C)c1cc(-c2ccc(N(c3cc(C(C)(C)C)cc(C(C)(C)C)c3)c3ccc4c(c3)C(C)(C)c3ccccc3-4)cc2)cc(C(C)(C)C)c1]

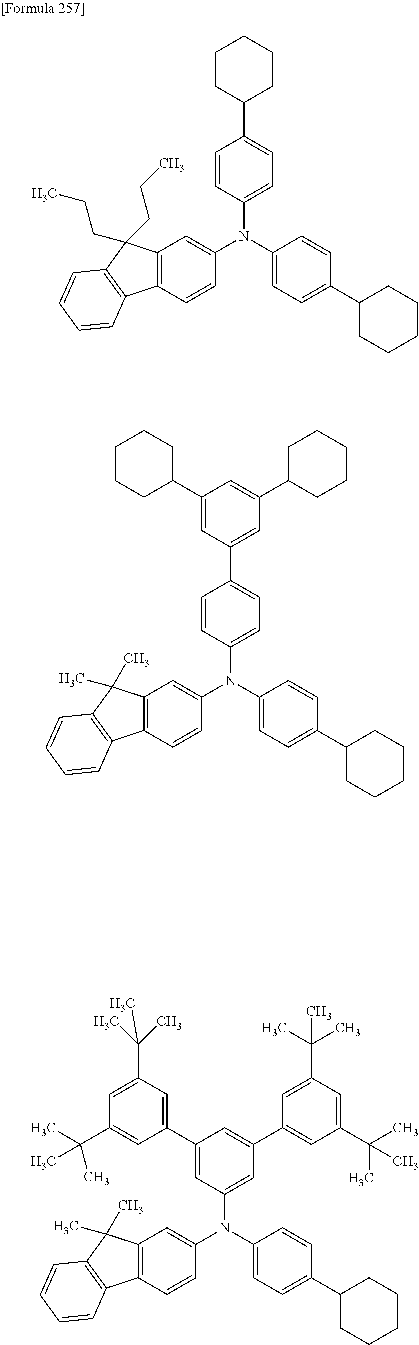 [SMILES: CCCC1(CCC)c2ccccc2-c2ccc(N(c3ccc(C4CCCCC4)cc3)c3ccc(C4CCCCC4)cc3)cc21 ; CC1(C)c2ccccc2-c2ccc(N(c3ccc(-c4cc(C5CCCCC5)cc(C5CCCCC5)c4)cc3)c3ccc(C4CCCCC4)cc3)cc21 ; CC(C)(C)c1cc(-c2cc(-c3cc(C(C)(C)C)cc(C(C)(C)C)c3)cc(N(c3ccc(C4CCCCC4)cc3)c3ccc4c(c3)C(C)(C)c3ccccc3-4)c2)cc(C(C)(C)C)c1]

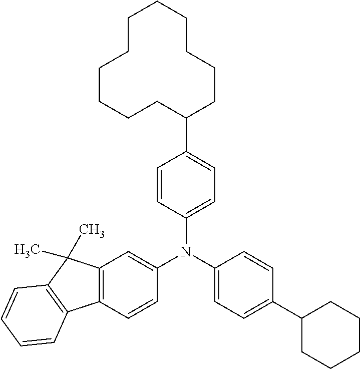 [SMILES: CC1(C)c2ccccc2-c2ccc(N(c3ccc(C4CCCCCCCCCCC4)cc3)c3ccc(C4CCCCC4)cc3)cc21]

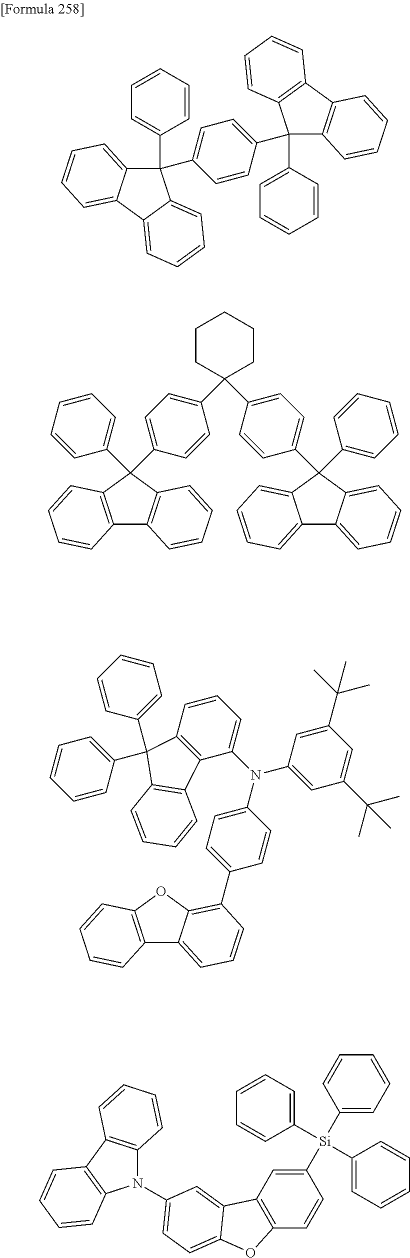

The fifth material as the additive material in an exemplary arrangement of the organic EL devices of the first, second, and third exemplary embodiments as well as the fifth material as the additive material in an exemplary arrangement of the organic EL devices of the fourth, fifth, and sixth exemplary embodiments are each a fluorine resin.

In an exemplary arrangement of the organic EL device of each of the exemplary embodiments (i.e., the first, second, third, fourth, fifth, and sixth exemplary embodiments), the fifth material as the additive material is at least one polymer selected from the group consisting of polytetrafluoroethylene (PTFE), tetrafluoroethylene/perfluoroalkyl vinyl ether copolymer (PFA), tetrafluoroethylene/hexafluoropropylene copolymer (FEP), tetrafluoroethylene/ethylene copolymer (ETFE), polyvinylidene fluoride (PVDF), polychlorotrifluoroethylene (PCTFE) and chlorotrifluoroethylene/ethylene copolymer (ECTFE).

In an exemplary arrangement of the organic EL device of each exemplary embodiment, the first anode-side organic layer contains poly(alkylene dioxythiophene) and at least one fluorine-containing colloid-forming polymeric acid. Poly(alkylene dioxythiophene) is poly(3,4-dioxythiophene). The fluorine-containing colloid-forming polymeric acid is a fluorine-containing polymer sulfonic acid, a fluorine-containing polymer carboxylic acid, a fluorine-containing polymer phosphoric acid, a fluorine-containing polymer acrylic acid, or a mixture thereof. The fluorine-containing colloid-forming polymeric acid is preferably a perfluorinated polymeric acid.

In an exemplary arrangement of the organic EL device of each exemplary embodiment, a colloid-forming polymeric acid is insoluble in water, and forms a colloid when dispersed in an aqueous medium. A molecular weight of a polymer acid is typically in a range from approximately 10,000 to approximately 4,000,000. In an exemplary arrangement of the organic EL device of each exemplary embodiment, a molecular weight of a polymer acid is in a range from approximately 100,000 to approximately 2,000,000. A diameter of a colloid particle is typically in a range from 2 nm to approximately 140 nm. In an exemplary arrangement of the organic EL device of each exemplary embodiment, a diameter of a colloid particle is in a range from 2 nm to approximately 30 nm. Any polymer acid capable of forming a colloid when dispersed in water is preferable. In an exemplary arrangement of the organic EL device of each exemplary embodiment, the colloid-forming polymeric acid is a polymer sulfonic acid. Examples of another usable polymer acid include a polymer phosphoric acid, a polymer carboxylic acid, a polymer acrylic acid, and a mixture thereof. The mixture is exemplified by a mixture containing a polymer sulfonic acid. In an exemplary arrangement of the organic EL device of each exemplary embodiment, the colloid-forming polymer sulfonic acid is perfluorinated. In an exemplary arrangement of the organic EL device of each exemplary embodiment, the colloid-forming polymer sulfonic acid is a perfluoro alkylene sulfonic acid.

In an exemplary arrangement of the organic EL device of each exemplary embodiment, the colloid-forming polymeric acid is a highly-fluorinated sulfonate polymer (FSA polymer). The wording “highly-fluorinated” means that: fluorine atoms account for at least approximately 50% of the total of halogens and hydrogen atoms contained in a polymer; fluorine atoms account for at least approximately 75% thereof in another exemplary embodiment; and fluorine atoms account for at least approximately 90% thereof in still another exemplary embodiment. In an exemplary arrangement of the organic EL device of each exemplary embodiment, the polymer is perfluorinated. A term “sulfonate functional group” means either a sulfonate group or a salt of a sulfonate group. In an exemplary arrangement of the organic EL device of each exemplary embodiment, the term means either an alkali metal or an ammonium salt. The sulfonate functional group is represented by a formula —SO₃X (where X represents a cation, which is also known as “counterion”). X may be H, Li, Na, K, or N(R₁)(R₂)(R₃)(R₄), in which R₁, R₂, R₃, and R₄ may be mutually the same or different. R₁, R₂, R₃, and R⁴ are each independently H, CH₃ or C₂H₅. In an exemplary arrangement of the organic EL device of each exemplary embodiment, X is H. In such a case, the polymer is said to be in an “acid form.” X may be polyvalent as represented by an ion such as Ca⁺⁺ and Al⁺⁺⁺. When a polyvalent counterion as generally represented by Mn⁺ is concerned, those skilled in the art will clearly understand that the number of sulfonate functional groups per counterion is equal to the valence number “n” of the counterion.

In an exemplary arrangement of the organic EL device of each exemplary embodiment, the FSA polymer has a polymer main chain to which repeating side chains having a cation-exchange group are bonded. The polymer may be a homopolymer or a copolymer of two or more monomers. A copolymer is typically formed including a non-functionalized monomer and a second monomer having a cation-exchange group or its precursor such as a sulfonyl fluoride group (—SO₂F) that can be subsequently hydrolyzed to a sulfonate functional group. For instance, a copolymer of a first fluorinated vinyl monomer and a second fluorinated vinyl monomer having a sulfonyl fluoride group (—SO₂F) may be used as the copolymer. Examples of possible monomers as the first fluorinated vinyl monomer include tetrafluoroethylene (TFE), hexafluoropropylene, fluorinated vinyl, fluorinated vinylidene, trifluoroethylene, chlorotrifluoroethylene, perfluoro (alkyl vinyl ether) and a combination thereof. TFE is preferable as the first fluorinated vinyl monomer.

In an exemplary arrangement of the organic EL device of each exemplary embodiment, examples of possible monomers as the second monomer include fluorinated vinyl ether having a sulfonate functional group or a precursor group, the groups being capable of providing a desirable side chain to the polymer. An additional monomer may be incorporated into the above polymers as necessary. The additional monomer is at least one monomer selected from the group consisting of ethylene, propylene and R—CH═CH₂ (where R is a perfluorinated alkyl group having 1 to 10 carbon atoms). The polymer may be a copolymer that is herein referred to as a random copolymer, i.e., a copolymer produced by polymerization where a relative concentration of the comonomer is kept as constant as possible so that a distribution of monomer units along a polymer chain consequently corresponds to relative concentrations and relative reactivity of the monomer units. A less random copolymer produced by changing the relative concentrations of monomers during polymerization may also be used. Such a polymer, which is referred to as a block copolymer, as disclosed in JP Patent Publication No. 2003-297582 may also be used.

In an exemplary arrangement of the organic EL device of each exemplary embodiment, the FSA polymer includes a highly-fluorinated carbon main chain, which may be perfluorinated in an exemplary arrangement, and a side chain represented by a formula (FP1) below.

—(O—CF₂CFR_{f})ₐ—O—CF₂CFR′_{f}SO₃X (FP1)

In the formula (FP1): R_{f} and R′_{f} are each independently F, Cl, or a perfluorinated alkyl group having 1 to 10 carbon atoms; a is 0, 1, or 2; and X is H, Li, Na, K, or N(R₁)(R₂)(R₃)(R₄), in which R₁, R₂, R₃, and R₄ are mutually the same or different, and R₁, R₂, R₃, and R⁴ are each independently H, CH₃ or C₂H₅. In an exemplary arrangement of the organic EL device of each exemplary embodiment, X in the formula (FP1) is H. Further, in an exemplary arrangement of the organic EL device of each exemplary embodiment, X in the formula (FP1) is polyvalent as described above.

In an exemplary arrangement of the organic EL device of each exemplary embodiment, examples of the FSA polymer include polymers disclosed in U.S. Pat. Nos. 3,282,875, 4,358,545, and 4,940,525. The FSA polymer is preferably exemplified by a polymer having a perfluorocarbon main chain and a side chain represented by a formula (FP2) below.

—O—CF₂CF(CF₃)—O—CF₂CF₂SO₃X (FP2)
where X in the formula (FP2) represents the same as X the formula (FP1).

The above type of the FSA polymer, which is disclosed in U.S. Pat. No. 3,282,875, can be produced by: copolymerizing tetrafluoroethylene (TFE) and perfluorinated vinyl ether CF₂═CF—O—CF₂CF(CF₃)—O—CF₂CF₂SO₂F (perfluoro (3,6-dioxa-4-methyl-7-octene sulfonyl fluoride)) (PDMOF); subsequently converting the sulfonyl fluoride group into a sulfonate group by hydrolysis; and converting the obtained group into a desired ion form by ion exchange as necessary. Such a polymer as disclosed in U.S. Pat. Nos. 4,358,545 and 4,940,525 has the side chains represented by —O—CF₂CF₂SO₃X where X is defined as above. This polymer can be produced by copolymerizing tetrafluoroethylene (TFE) and perfluorinated vinyl ether CF₂═CF—O—CF₂CF₂SO₂F (perfluoro (3-oxa-4-pentene sulfonyl fluoride)) (POPF), followed by hydrolysis and, as necessary, further ion exchange.

In an exemplary arrangement of the organic EL device of each exemplary embodiment, the FSA polymer typically has an ion-exchange ratio of less than approximately 33. The “ion-exchange ratio” or “IXR” herein is defined as the number of carbon atoms included in a polymer main chains which are relevant to a cation-exchange group. IXR may be changed within a range of less than approximately 33 to be suitable for a specific use. In an exemplary arrangement of the organic EL device of exemplary embodiment, IXR is in a range from approximately 3 to approximately 33. In an exemplary arrangement of the organic EL device of exemplary embodiment, IXR is in a range from approximately 8 to approximately 23.

Cation-exchange capacity of a polymer is often represented in equivalent weight (EW). The equivalent weight (EW) is defined as a weight of a polymer in an acid form required for neutralizing 1 equivalent weight of sodium hydrate. When a polymer is a sulfonate polymer that has a perfluorocarbon main chain and side chains of —O—CF₂—CF(CF₃)—O—CF₂—CF₂—SO₃H (or a salt thereof), an equivalent-weight range corresponding to IXR in the range from approximately 8 to approximately 23 is from approximately 750 EW to approximately 1500 EW. IXR of this polymer can be associated with the equivalent weight by using a formula of 50 IXR+344=EW.

Although the same IXR range may be applied to, for instance, a sulfonate polymer disclosed in U.S. Pat. Nos. 4,358,545 and 4,940,525, for instance, a polymer having the side chains of —O—CF₂CF₂SO₃H (or a salt thereof), an equivalent weight thereof is slightly lower due to lower molecular weights of monomer units containing cation-exchange groups. A preferable IXR range from approximately 8 to approximately 23 corresponds to an equivalent weight range from approximately 575 EW to approximately 1325 EW. IXR of this polymer can be associated with the equivalent weight by using a formula of 50 IXR+178=EW.

FSA polymer is producible as a colloidal aqueous dispersion solution. The FSA polymer may be a dispersion solution in another medium. The medium, which is not limited, is exemplified by alcohol, water-soluble ether such as tetrahydrofuran, a mixture of water-soluble ether, and a combination thereof. In producing a dispersion solution, the polymer may be used in acid form. U.S. Pat. Nos. 4,433,082, 6,150,426, and International Publication No. 03/006537 each disclose a producing method of an aqueous alcoholic dispersion solution. After the dispersion solution is produced, a concentration and compositions of the dispersion solution can be adjusted by a method publicly known in the art.

An aqueous dispersion solution of a colloid-forming polymer acid such as the FSA polymer typically has a colloid having as small particle diameter as possible and as small EW as possible, as long as a stable colloid is formed.

An aqueous dispersion solution of the FSA polymer is commercially available as Nafion (Registered Trademark) dispersion solution from E.I. du Pont de Nemours and Company (Wilmington, DE).

### Emitting Region

The emitting region is disposed between the hole transporting zone and the cathode. The emitting region includes at least one emitting layer. The organic EL device of each exemplary embodiment includes a first emitting region as the emitting region.

In an exemplary arrangement of the organic EL device of each exemplary embodiment, the first emitting region includes the emitting layer containing a host material and a luminescent compound that emits light having a maximum peak wavelength of 500 nm or less.

In an exemplary arrangement of the organic EL device of each exemplary embodiment, a full width at half maximum at the maximum peak of the luminescent compound is in a range from 1 nm to 30 nm.

### First Emitting Layer

In an exemplary arrangement of the organic EL device of each exemplary embodiment, a first emitting layer is included as the at least one emitting layer. In an exemplary arrangement of the organic EL device of each exemplary embodiment, the first emitting layer contains a first host material. The first host material, which is not particularly limited, may be selected from the group consisting of a compound represented by a formula (H1) below, a compound represented by a formula (H10), a compound represented by a formula (H20), and a first compound described below.

In an exemplary arrangement of the organic EL device of each exemplary embodiment, the first emitting layer contains a first luminescent compound. The first luminescent compound is not particularly limited.

In an exemplary arrangement of the organic EL device of each exemplary embodiment, the first emitting layer contains the first host material and the first luminescent compound.

The first luminescent compound is preferably a compound that emits light having a maximum peak wavelength of 500 nm or less, more preferably a compound that emits light having a maximum peak wavelength in a range from 430 nm to 480 nm. The first luminescent compound is preferably a fluorescent compound that emits fluorescence having the maximum peak wavelength of 500 nm or less, more preferably a fluorescent compound that emits fluorescence having the maximum peak wavelength in a range from 430 nm to 480 nm.

In an exemplary arrangement of the organic EL device of each exemplary embodiment, the first emitting layer contains the first host material and the first luminescent compound that emits light having the maximum peak wavelength of 500 nm or less.

In an exemplary arrangement of the organic EL device of each exemplary embodiment, the full width at half maximum of the maximum peak of the first luminescent compound is in a range from 1 nm to 30 nm.

In an exemplary arrangement of the organic EL device of each exemplary embodiment, the first luminescent compound is a compound containing no azine ring structure in a molecule.

In an exemplary arrangement of the organic EL device of each exemplary embodiment, the first luminescent compound is preferably not a boron-containing complex, more preferably not a complex.

As a fluorescent compound that emits blue fluorescence and is usable for the first emitting layer, for instance, compounds such as a pyrene derivative, a styrylamine derivative, a chrysene derivative, a fluoranthene derivative, a fluorene derivative, a diamine derivative, and a triarylamine derivative may be used.

Herein, the blue light emission refers to a light emission in which a maximum peak wavelength of emission spectrum is in a range from 430 nm to 500 nm.

In an exemplary arrangement of the organic EL device of each exemplary embodiment, the first emitting region includes two or more emitting layers.

In an exemplary arrangement of the organic EL device of each exemplary embodiment, when the first emitting region includes two or more emitting layers, the two or more emitting layers are each an emitting layer that emits fluorescence.

That is, in an exemplary arrangement of the organic EL device of each exemplary embodiment, all the emitting layers included in the first emitting region emit fluorescence.

In an exemplary arrangement of the organic EL device of each exemplary embodiment, the first emitting layer contains no metal complex. In an exemplary arrangement of the organic EL device of each exemplary embodiment, the first emitting layer contains no boron-containing complex.

In an exemplary arrangement of the organic EL device of each exemplary embodiment, the first emitting layer contains no phosphorescent material (dopant material).

In an exemplary arrangement of the organic EL device of each exemplary embodiment, the first emitting layer contains no heavy-metal complex and no phosphorescent rare earth metal complex. Examples of the heavy-metal complex herein include an iridium complex, osmium complex, and platinum complex.

A method of measuring the maximum peak wavelength of the compound is as follows. A toluene solution of a measurement target compound at a concentration of 5 μmol/L is prepared and put in a quartz cell. An emission spectrum (ordinate axis: luminous intensity, abscissa axis: wavelength) of the thus-obtained sample is measured at a normal temperature (300K). The emission spectrum can be measured using a spectrophotometer (apparatus name: F-7000) produced by Hitachi High-Tech Science Corporation. It should be noted that the apparatus for measuring the emission spectrum is not limited to the apparatus used herein.

A peak wavelength of the emission spectrum exhibiting the maximum luminous intensity is defined as the maximum peak wavelength. Herein, the maximum peak wavelength of fluorescence is occasionally referred to as a maximum fluorescence peak wavelength (FL-peak).

In an emission spectrum of the first luminescent compound, where a peak exhibiting a maximum luminous intensity is defined as a maximum peak and a height of the maximum peak is defined as 1, heights of other peaks appearing in the emission spectrum are preferably less than 0.6. It should be noted that the peaks in the emission spectrum are defined as local maximum values.

Moreover, in the emission spectrum of the first luminescent compound, the number of peaks is preferably less than three.

In an exemplary arrangement of the organic EL device of each exemplary embodiment, the first emitting layer contains 0.5 mass % or more of the first luminescent compound with respect to a total mass of the first emitting layer.

The first emitting layer contains the first luminescent compound preferably at 10 mass % or less, more preferably at 7 mass % or less, and still more preferably at 5 mass % less, with respect to the total mass of the first emitting layer.

Herein, the “host material” refers to, for instance, a material that accounts for “50 mass % or more of the layer”.

In an exemplary arrangement of the organic EL device of each exemplary embodiment, the first emitting layer contains the first host material preferably at 60 mass % or more, more preferably at 70 mass % or more, still more preferably at 80 mass % or more, still further more preferably at 90 mass % or more, and yet still further more preferably at 95 mass % or more, with respect to the total mass of the first emitting layer.

The first emitting layer preferably contains the first host material at 99.5 mass % or less with respect to the total mass of the first emitting layer.

When the first emitting layer contains the first host material and the first luminescent compound, the upper limit of a total of the content ratios of the first host material and the first luminescent compound is 100 mass %.

In the organic EL device of each exemplary embodiment, the first emitting layer may further contain any other material than the first host material and the first luminescent compound.

The first emitting layer may contain a single type of the first host material or may contain two or more types of the first host material. The first emitting layer may contain a single type of the first luminescent compound or may contain two or more types of the first luminescent compound.

### First Host Material

In an exemplary arrangement of the organic EL device of each exemplary embodiment, the first host material has at least one deuterium atom.

In an exemplary arrangement of the organic EL device of each exemplary embodiment, the first host material has no deuterium atom.

### Compound Represented by Formula (H1)

In an exemplary arrangement of the organic EL device of each exemplary embodiment, the first host material is a compound represented by a formula (H1) below.

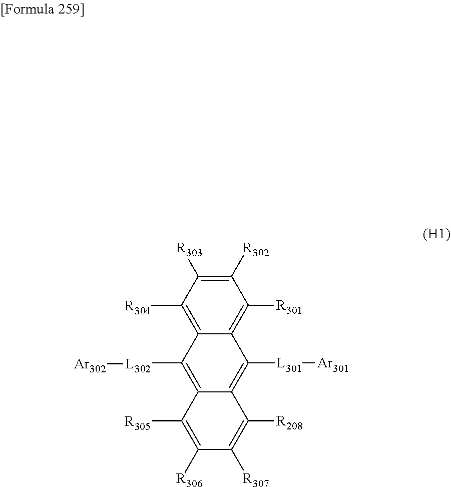

In the formula (H1):
R₃₀₁ to R₃₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₃₀₁ and L₃₀₂ are each independently a single bond, a substituted or unsubstituted arylene group having 6 to 50 ring carbon atoms, or a substituted or unsubstituted divalent heterocyclic group having 5 to 50 ring atoms; and
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms.

In the compound represented by the formula (H1), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an arrangement of the organic EL device according to each of the exemplary embodiments, Ar₃₀₁ and Ar₃₀₂ are each independently a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

In an exemplary arrangement of the organic EL device according to each of the exemplary embodiments, L₃₀₁ is a single bond or an unsubstituted arylene group having 6 to 22 ring carbon atoms; and Ar₃₀₁ is a substituted or unsubstituted aryl group having 6 to 22 ring carbon atoms.

In an exemplary arrangement of the organic EL device of each exemplary embodiment, R₃₀₁ to R₃₀₈ are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In an exemplary arrangement of the organic EL device of each exemplary embodiment, R₃₀₁ to R₃₀₈ are each a hydrogen atom.

In an exemplary arrangement of the organic EL device of each exemplary embodiment, L₃₀₂ is a single bond and Ar₃₀₂ is an unsubstituted phenyl group.

In an exemplary arrangement of the organic EL device of each exemplary embodiment, L₃₀₂ is a single bond and Ar₃₀₂ is an unsubstituted 2-naphthyl group.

In an exemplary arrangement of the organic EL device of each exemplary embodiment, L₃₀₂ is a single bond and Ar₃₀₂ is an unsubstituted 1-naphthyl group.

In an exemplary arrangement of the organic EL device of each exemplary embodiment, L₃₀₂ is an unsubstituted p-phenylene group and Ar₃₀₂ is an unsubstituted phenyl group.

In an exemplary arrangement of the organic EL device of each exemplary embodiment, L₃₀₂ is an unsubstituted m-phenylene group and Ar₃₀₂ is an unsubstituted phenyl group.

In an exemplary arrangement of the organic EL device of each exemplary embodiment, L₃₀₂ is an unsubstituted o-phenylene group and Ar₃₀₂ is an unsubstituted phenyl group.

In an exemplary arrangement of the organic EL device of each exemplary embodiment, L₃₀₂ is an unsubstituted p-phenylene group and Ar₃₀₂ is an unsubstituted 1-naphthyl group.

In an exemplary arrangement of the organic EL device of each exemplary embodiment, L₃₀₂ is an unsubstituted p-phenylene group and Ar₃₀₂ is an unsubstituted 2-naphthyl group.

In an exemplary arrangement of the organic EL device of each exemplary embodiment, L₃₀₂ is an unsubstituted 1.4-naphthalenediyl group and Ar₃₀₂ is an unsubstituted phenyl group.

In an exemplary arrangement of the organic EL device of each exemplary embodiment, L₃₀₂ is an unsubstituted m-phenylene group and Ar₃₀₂ is an unsubstituted 2-naphthyl group.

In an exemplary arrangement of the organic EL device of each exemplary embodiment, R₃₀₁ to R₃₀₈ each independently have at least one deuterium atom.

In an exemplary arrangement of the organic EL device of each exemplary embodiment, L₃₀₁, L₃₀₂, Ar₃₀₁, and Ar₃₀₂ each independently have at least one deuterium atom.

### Compound Represented by Formula (H10)

In an exemplary arrangement of the organic EL device of each exemplary embodiment, the first host material is a compound represented by a formula (H10) below.

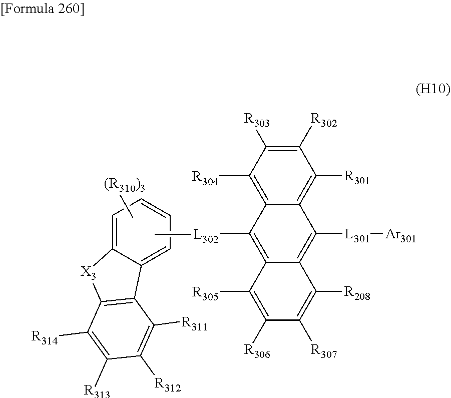

In the formula (H10): Ar₃₀₁, R₃₀₁ to R₃₀₈, L₃₀₁, and L₃₀₂ each independently represent the same as Ar₃₀₁, R₃₀₁ to R₃₀₈, L₃₀₁, and L₃₀₂ in the formula (H1);
X₃ is an oxygen atom or a sulfur atom;
at least one combination of adjacent two or more of three R₃₁₀ are mutually bonded to form a substituted or unsubstituted monocyclic ring, mutually bonded to form a substituted or unsubstituted fused ring, or not mutually bonded;
at least one combination of adjacent two or more of R₃₁₁ to R₃₁₄ are mutually bonded to form a substituted or unsubstituted monocyclic ring, or mutually bonded to form a substituted or unsubstituted fused ring; and
R₃₁₀ to R₃₁₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the compound represented by the formula (H10), R₉₀₁, R₉₀₂, R₉₀₃, R₉₀₄, R₉₀₅, R₉₀₆, and R₉₀₇ each independently represent the same as R₉₀₁, R₉₀₂, R₉₀₃, R₉₀₄, R₉₀₅, R₉₀₆, and R₉₀₇ in the formula (H1).

In an exemplary arrangement of the organic EL device of each exemplary embodiment, at least one combination of adjacent two or more of R₃₁₁ to R₃₁₄ in the formula (H10) are bonded to each other to form a substituted or unsubstituted benzene ring.

In an exemplary arrangement of the organic EL device of each exemplary embodiment, R₃₀₁ to R₃₀₈ each independently have at least one deuterium atom.

In an exemplary arrangement of the organic EL device of each exemplary embodiment, L₃₀₁, L₃₀₂, Ar₃₀₁, and R₃₁₀ to R₃₁₄ each independently have at least one deuterium atom.

In an exemplary arrangement of the organic EL device of each exemplary embodiment, the first host material is a compound represented by a formula (H31), (H32) or (H33) below.

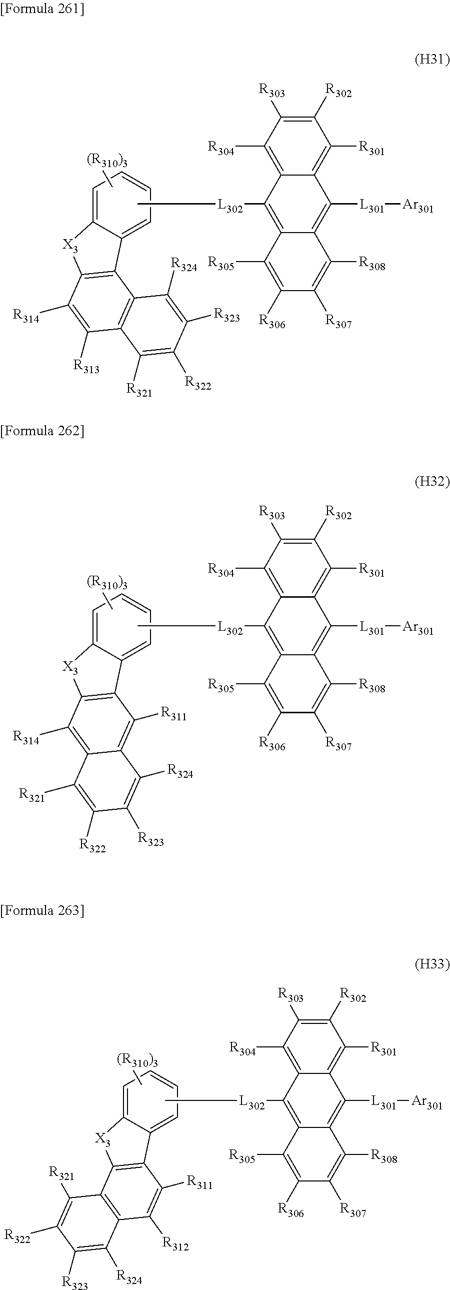

In the formulae (H31), (H32) and (H33), X₃, R₃₀₁ to R₃₀₈, R₃₁₀ to R₃₁₄, L₃₀₁, L₃₀₂, and Ar₃₀₁ each independently represent the same as X₃, R₃₀₁ to R₃₀₈, R₃₁₀ to R₃₁₄, L₃₀₁, L₃₀₂, and Ar₃₀₁ in the formula (H10); and
R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, the first host material is a compound represented by a formula (H301) or (H302) below.

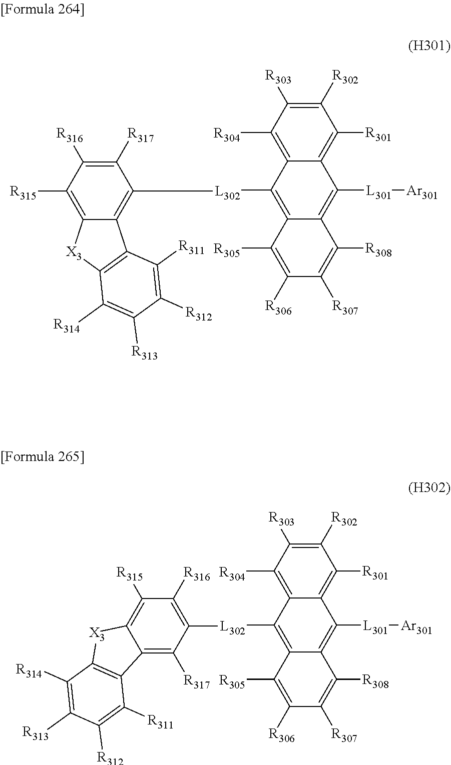

In the formulae (H301) and (H302), X₃, R₃₀₁ to R₃₀₈, R₃₁₁ to R₃₁₄, L₃₀₁, L₃₀₂, and Ar₃₀₁ each independently represent the same as X₃, R₃₀₁ to R₃₀₈, R₃₁₁ to R₃₁₄, L₃₀₁, L₃₀₂, and Ar₃₀₁ in the formula (H10);
at least one combination of adjacent two or more of R₃₁₅ to R₃₁₇ are mutually bonded to form a substituted or unsubstituted monocyclic ring, mutually bonded to form a substituted or unsubstituted fused ring, or not mutually bonded; and
R₃₁₅ to R₃₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, the first host material is a compound represented by a formula (H311), (H312), (H321), (H322), (H331), or (H332) below.

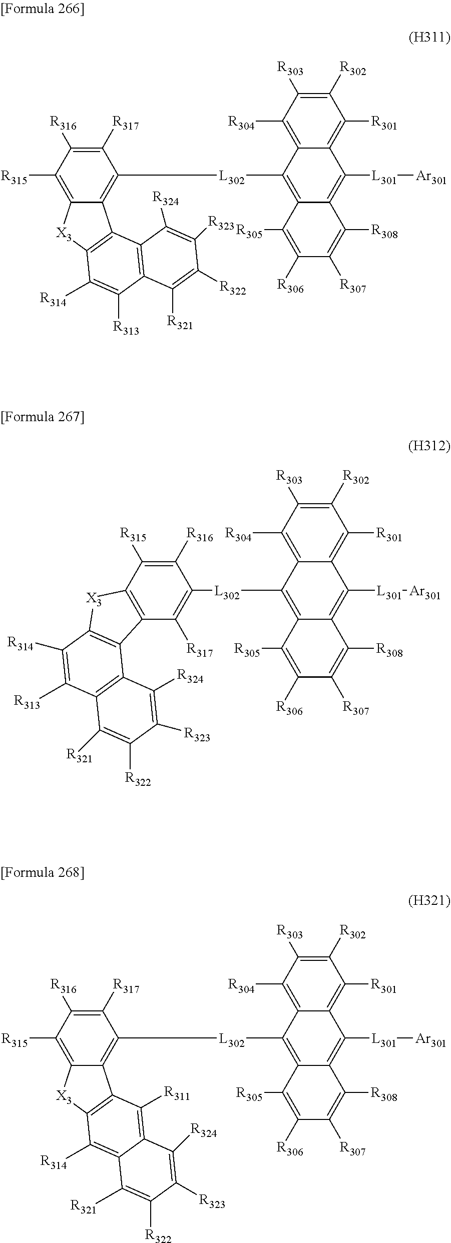

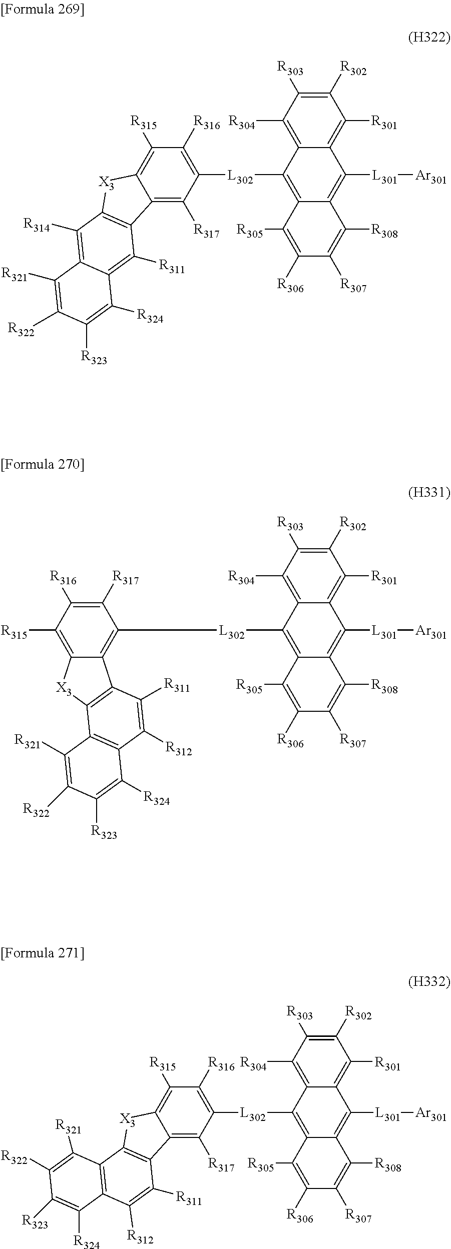

In the formulae (H311), (H312), (H321), (H322), (H331) and (H332), X₃, R₃₀₁ to R₃₀₈, L₃₀₁, L₃₀₂, and Ar₃₀₁ each independently represent the same as X₃, R₃₀₁ to R₃₀₈, L₃₀₁, L₃₀₂, and Ar₃₀₁ in the formula (H10); and
R₃₁₁ to R₃₁₇ and R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, L₃₀₁ and L₃₀₂ of the first host material are each independently a single bond or a substituted or unsubstituted arylene group having 6 to 14 ring carbon atoms.

In an exemplary arrangement of the organic EL device of each exemplary embodiment, L₃₀₁ and L₃₀₂ of the first host material are each independently a single bond, a substituted or unsubstituted phenylene group, or a substituted or unsubstituted naphthylene group.

In an exemplary arrangement of the organic EL device of each exemplary embodiment, L₃₀₁ and L₃₀₂ of the first host material are each a single bond.

In an exemplary arrangement of the organic EL device of each exemplary embodiment, the first host material is a compound represented by a formula (H313), (H314), (H323), (H324), (H333), or (H334) below.

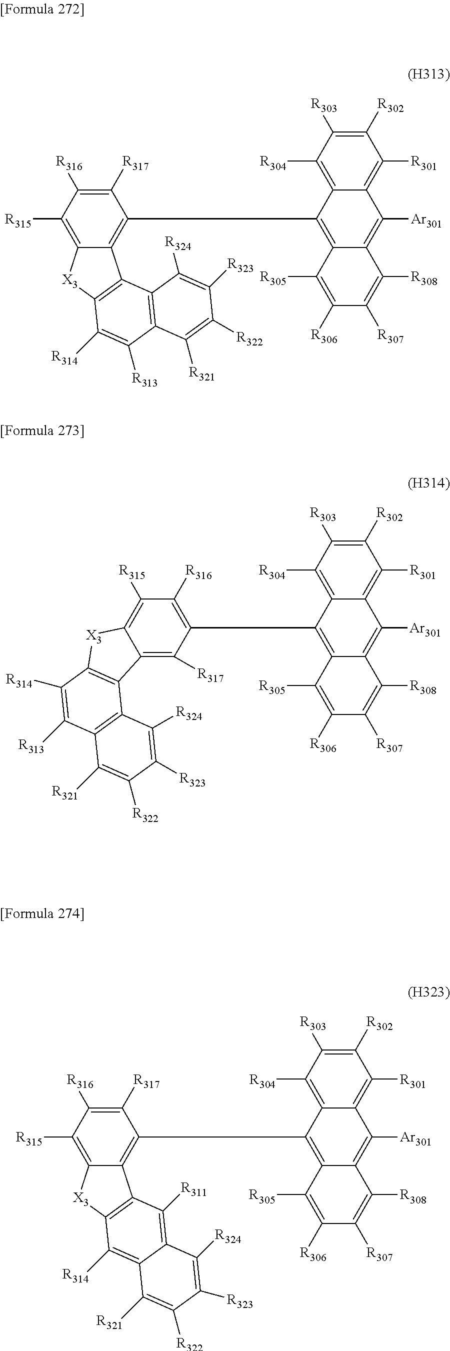

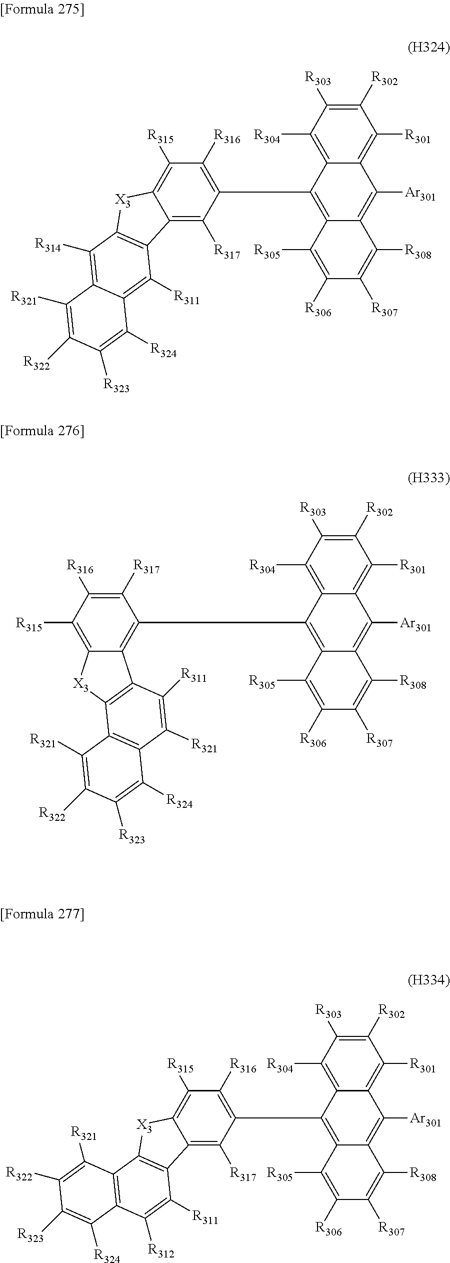

In the formulae (H313), (H314), (H323), (H324), (H333) and (H334), X₃, R₃₀₁ to R₃₀₈, and Ar₃₀₁ each independently represent the same as X₃, R₃₀₁ to R₃₀₈, and Ar₃₀₁ in the formula (H10); and
R₃₁₁ to R₃₁₇, and R₃₂₁ to R₃₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, R₃₁₁ to R₃₁₇ and R₃₂₁ to R₃₂₄ of the first host material are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, R₃₁₁ to R₃₁₇ and R₃₂₁ to R₃₂₄ of the first host material are each independently a hydrogen atom, a substituted or unsubstituted alkyl group having 1 to 50 carbon atoms, or a substituted or unsubstituted aryl group having 6 to 50 ring carbon atoms.

In an exemplary arrangement of the organic EL device of each exemplary embodiment, Ar₃₀₁ of the first host material is a substituted or unsubstituted aryl group having 6 to 50 ring carbon atoms.

In an exemplary arrangement of the organic EL device of each exemplary embodiment, Ar₃₀₁ of the first host material is a group represented by a formula (a1), (a2), (a3), or (a4) below.

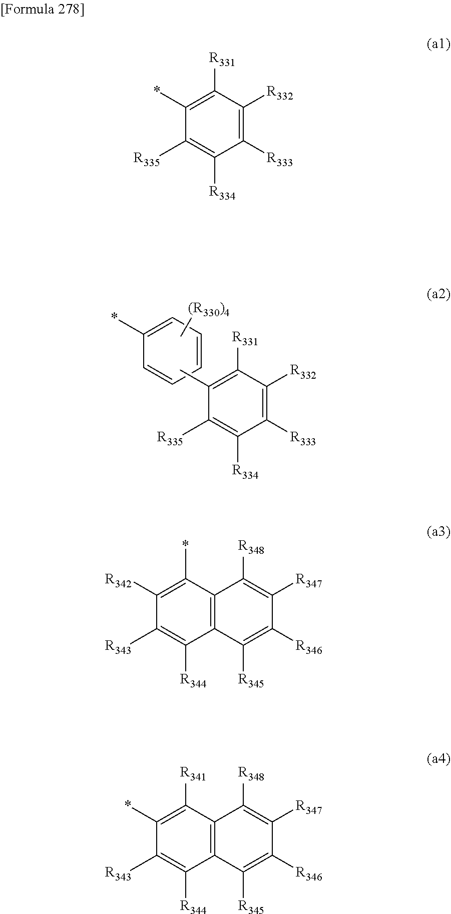

In the formulae (a1), (a2), (a3) and (a4):
at least one combination of adjacent two or more of R₃₃₀ to R₃₃₅ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₈ are mutually bonded to form a substituted or unsubstituted monocyclic ring, mutually bonded to form a substituted or unsubstituted fused ring, or not mutually bonded;
R₃₃₀ to R₃₃₅ and R₃₄₁ to R₃₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
a plurality of R₃₃₀ are mutually the same or different; and
* each represent a bonding position to L₃₀₁.

In an exemplary arrangement of the organic EL device of each exemplary embodiment, Ar₃₀₁ of the first host material is a group represented by the formula (a1) or (a2).

In an exemplary embodiment of the organic EL device of each exemplary embodiment, R₃₃₀ to R₃₃₅ and R₃₄₁ to R₃₄₈ of the first host material are each a hydrogen atom.

In an exemplary arrangement of the organic EL device of each exemplary embodiment, X₃ of the first host material is an oxygen atom.

In an exemplary arrangement of the organic EL device of each exemplary embodiment, R₃₀₁ to R₃₀₈ of the first host material are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary arrangement of the organic EL device of each exemplary embodiment, R₃₀₁ to R₃₀₈ of the first host material are each independently a hydrogen atom, a substituted or unsubstituted alkyl group having 1 to 50 carbon atoms, or a substituted or unsubstituted aryl group having 6 to 50 ring carbon atoms.

In an exemplary arrangement of the organic EL device of each exemplary embodiment, R₃₀₁ to R₃₀₈ of the first host material are each a hydrogen atom.

Compound Represented by Formula (H20) In an exemplary arrangement of the organic EL device of each exemplary embodiment, the first host material is a compound represented by a formula (H20) below.

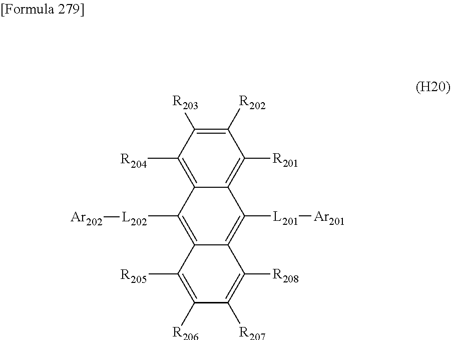

In the formula (H20): R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a group represented by a formula (H21) below.

[Formula 280]

-L₂₀₃-Ar₂₀₃ (H21)

In the formulae (H20) and (H21):
L₂₀₁, L₂₀₂, and L₂₀₃ are each independently a single bond, or a substituted or unsubstituted arylene group having 6 to 50 ring carbon atoms; and
Ar₂₀₁, Ar₂₀₂, and Ar₂₀₃ are each independently a substituted or unsubstituted aryl group having 6 to 50 ring carbon atoms.
   In the formula (H20), R₉₀₁, R₉₀₂, R₉₀₃, R₉₀₄, R₉₀₅, R₉₀₆, and R₉₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device of each exemplary embodiment, Ar₂₀₁, Ar₂₀₂, and Ar₂₀₃ are each independently a phenyl group, naphthyl group, phenanthryl group, biphenyl group, terphenyl group, diphenylfluorenyl group, dimethylfluorenyl group, benzodiphenylfluorenyl group, benzodimethylfluorenyl group, dibenzofuranyl group, dibenzothienyl group, naphthobenzofuranyl group, or naphthobenzothienyl group.

In an exemplary arrangement of the organic EL device of each exemplary embodiment, R₂₀₁ to R₂₀₈ each independently have at least one deuterium atom.

In an exemplary arrangement of the organic EL device of each exemplary embodiment, L₂₀₁, L₂₀₂, L₂₀₃, Ar₂₀₁, Ar₂₀₂, and Ar₂₀₃ each independently have at least one deuterium atom.

In an exemplary arrangement of the organic EL device of each exemplary embodiment, the compound represented by the formula (H20) is a compound represented by a formula (201), a formula (202), a formula (203), a formula (204), a formula (205), a formula (206), a formula (207), a formula (208), or a formula (209) below.

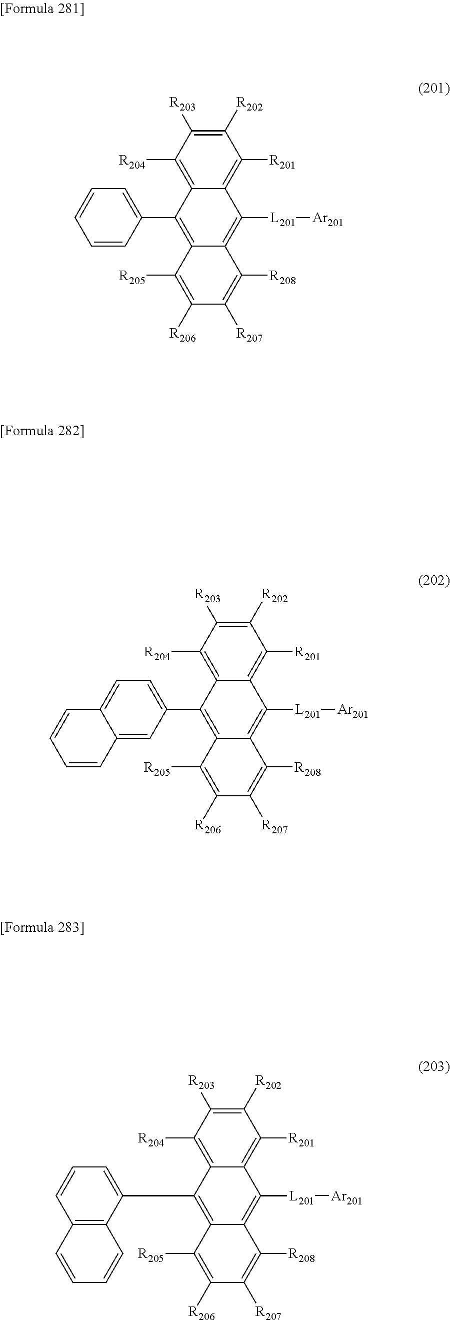

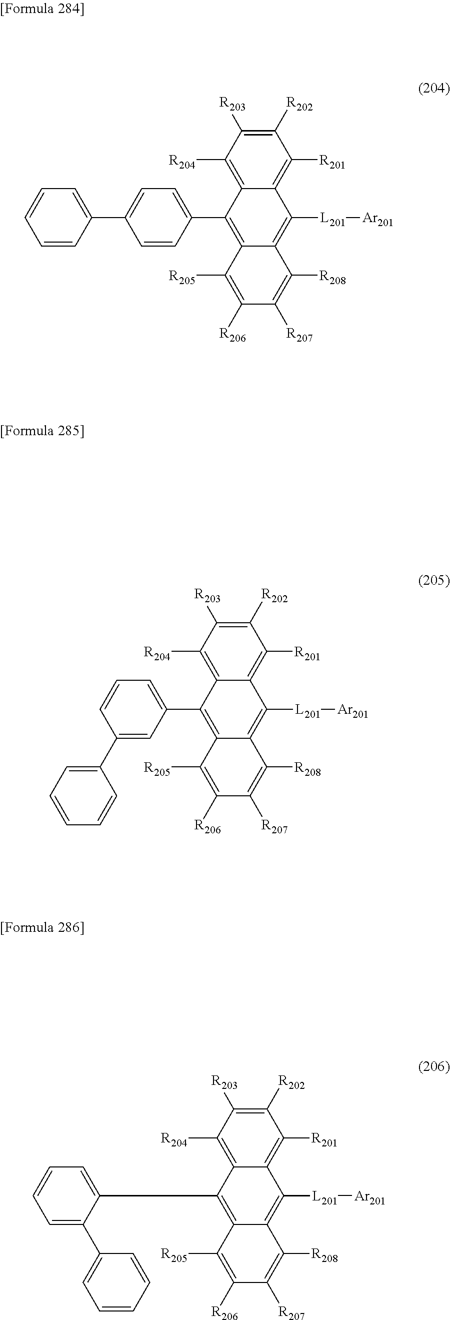

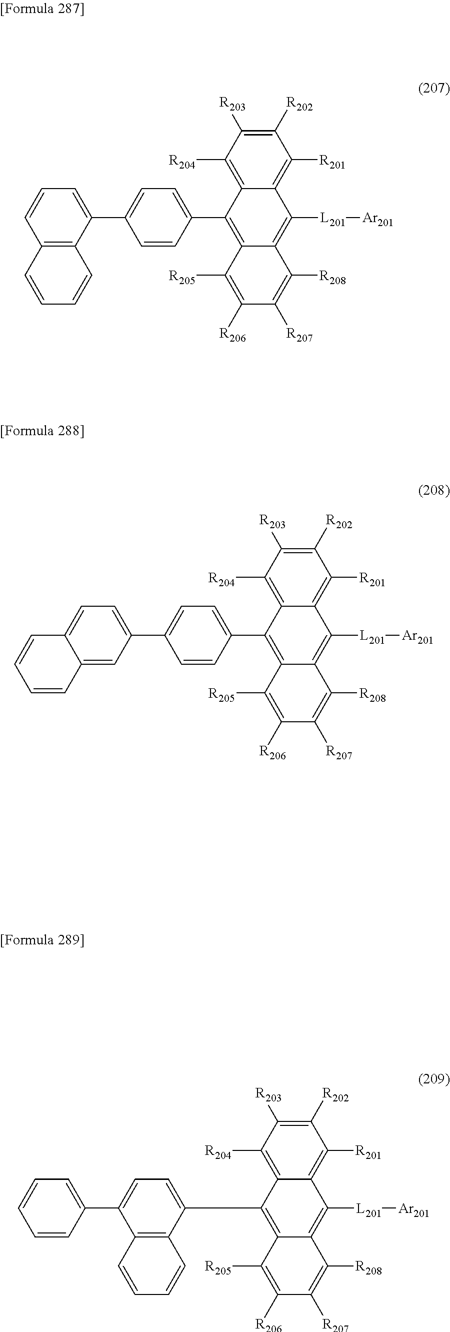

In the formulae (201) to (209), L₂₀₁ and Ar₂₀₁ respectively represent the same as L₂₀₁ and Ar₂₀₁ in the formula (H20); and R₂₀₁ to R₂₀₈ each independently represent the same as R₂₀₁ to R₂₀₈ in the formula (H20).

In an exemplary arrangement of the organic EL device of each exemplary embodiment, the compound represented by the formula (H20) is a compound represented by a formula (221), a formula (222), a formula (223), a formula (224), a formula (225), a formula (226), a formula (227), a formula (228), or a formula (229) below.

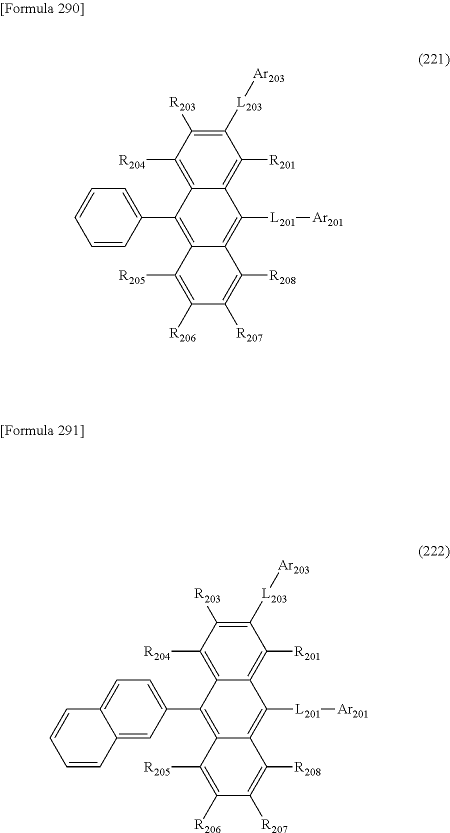

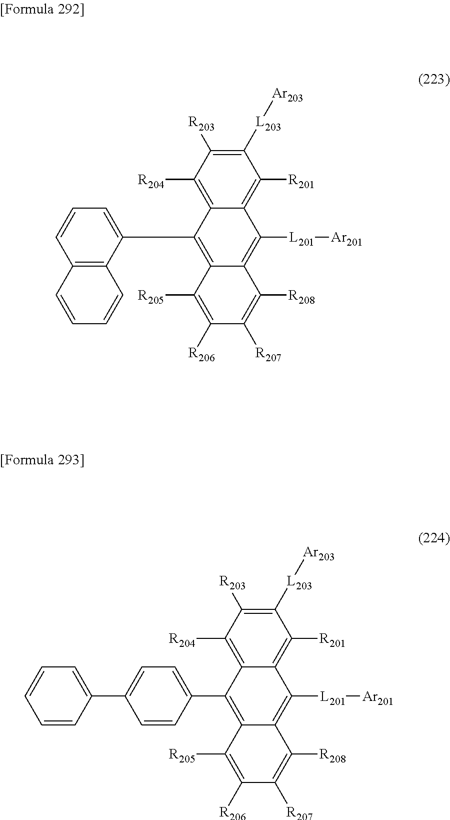

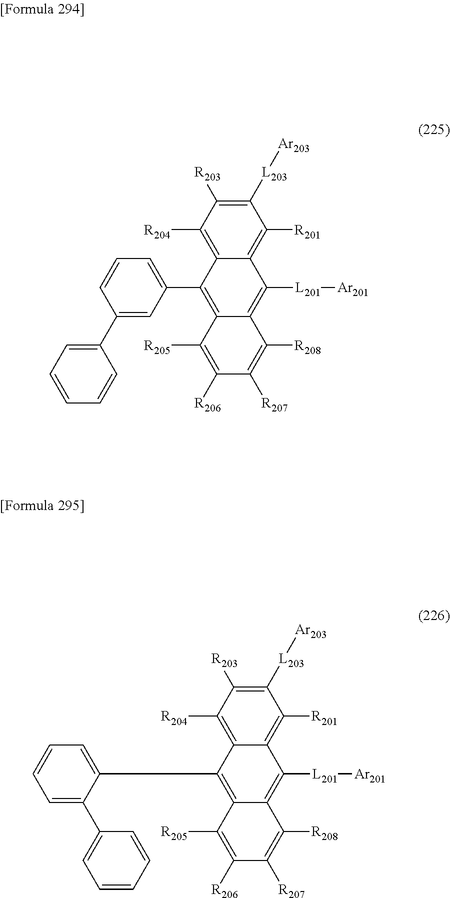

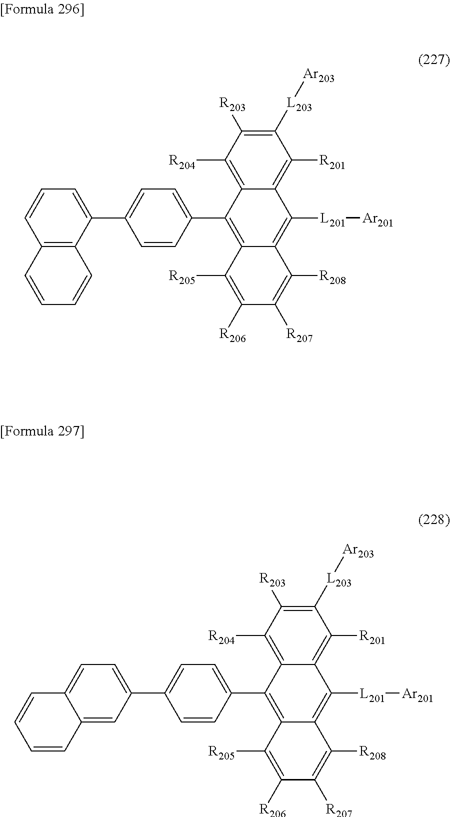

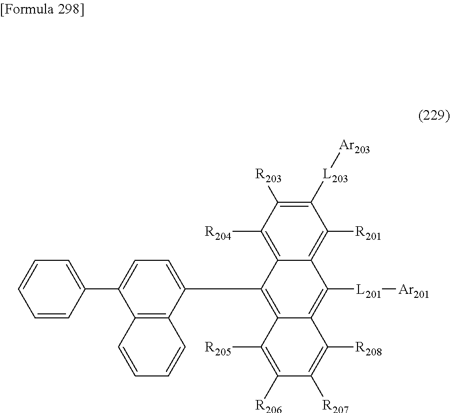

In the formulae (221), (222), (223), (224), (225), (226), (227), (228), and (229):
R₂₀₁ and R₂₀₃ to R₂₀₈ each independently represent the same as R₂₀₁ and R₂₀₃ to R₂₀₈ in the formula (H20);
L₂₀₁ and Ar₂₀₁ respectively represent the same as L₂₀₁ and Ar₂₀₁ in the formula (H20);
L₂₀₃ represents the same as Laos in the formula (H21);
L₂₀₃ and L₂₀₁ are mutually the same or different;
Ar₂₀₃ represents the same as Ar₂₀₃ in the formula (H21); and
Ar₂₀₃ and Ar₂₀₁ are mutually the same or different.

In an exemplary arrangement of the organic EL device of each exemplary embodiment, the compound represented by the formula (H20) is a compound represented by a formula (241), a formula (242), a formula (243), a formula (244), a formula (245), a formula (246), a formula (247), a formula (248), or a formula (249) below.

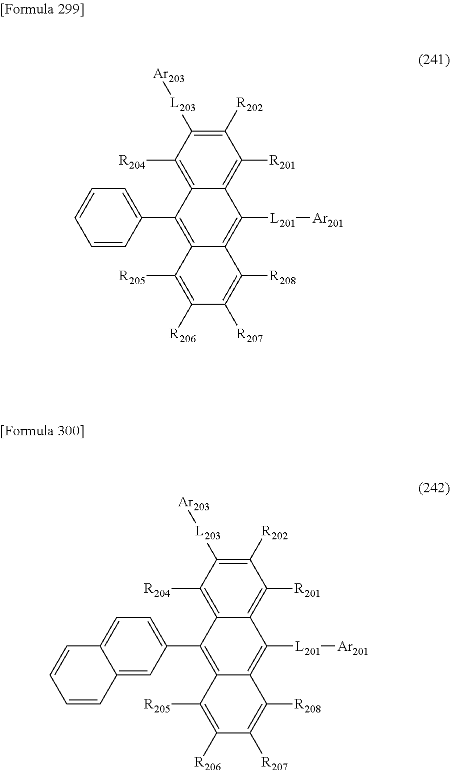

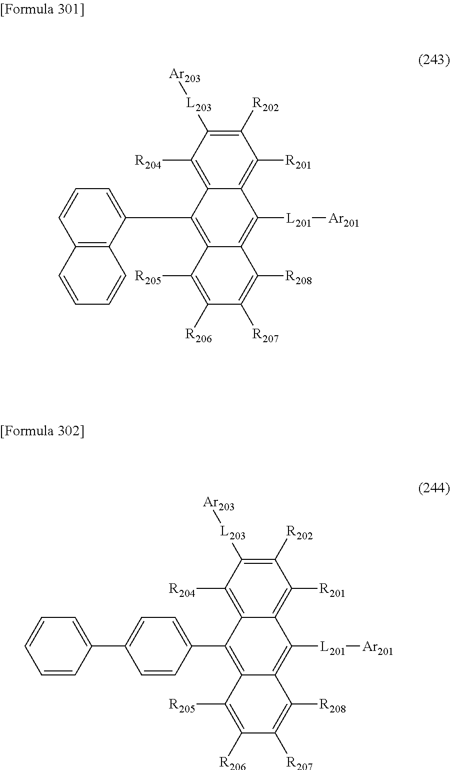

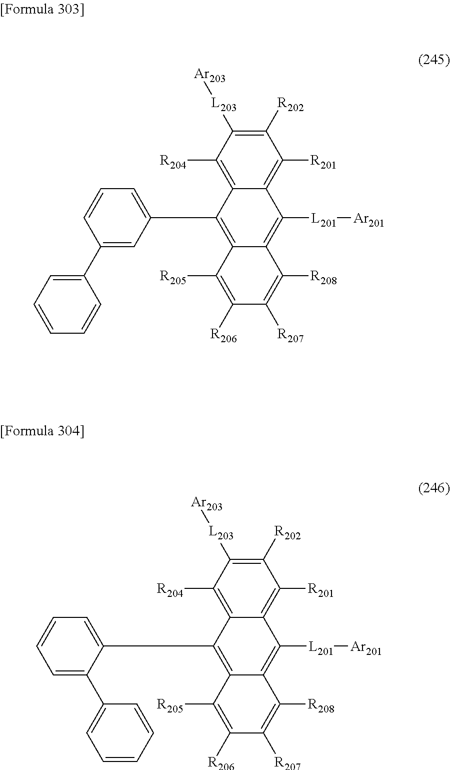

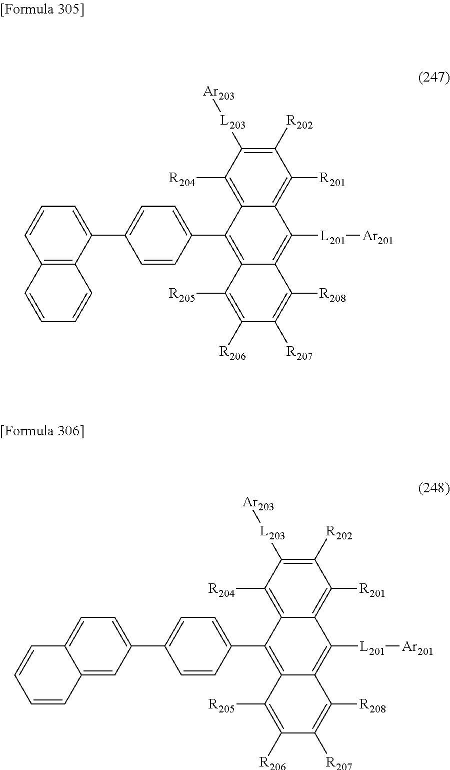

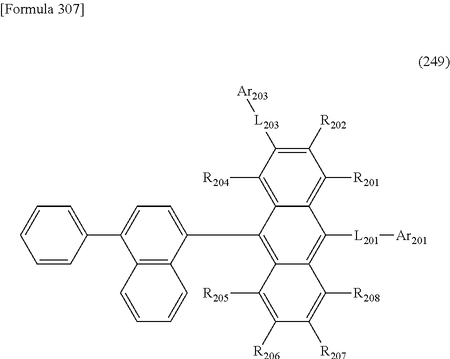

In the formulae (241), (242), (243), (244), (245), (246), (247), (248), and (249):
R₂₀₁, R₂₀₂ and R₂₀₄ to R₂₀₈ each independently represent the same as R₂₀₁, R₂₀₂ and R₂₀₄ to R₂₀₈ in the formula (H20);
L₂₀₁ and Ar₂₀₁ respectively represent the same as L₂₀₁ and Ar₂₀₁ in the formula (H20);
L₂₀₃ represents the same as L₂₀₃ in the formula (H21);
L₂₀₃ and L₂₀₁ are mutually the same or different;
Ar₂₀₃ represents the same as Ar₂₀₃ in the formula (H21); and
Ar₂₀₃ and Ar₂₀₁ are mutually the same or different.

In an exemplary arrangement of the organic EL device of each exemplary embodiment, R₂₀₁ to R₂₀₈ in the formula (H20) are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In an exemplary arrangement of the organic EL device of each exemplary embodiment, in the formula (H20), L₂₀₁ is a single bond or an unsubstituted arylene group having 6 to 22 ring carbon atoms and Ar₂₀₁ is a substituted or unsubstituted aryl group having 6 to 22 ring carbon atoms.

In an exemplary arrangement of the organic EL device of each exemplary embodiment, R₂₀₁ to R₂₀₈ that are substituents of an anthracene skeleton in the formula (H20) are preferably hydrogen atoms in terms of preventing inhibition of intermolecular interaction and inhibiting decrease in electron mobility. However, R₂₀₁ to R₂₀₈ may be a substituted or unsubstituted aryl group having 6 to 50 ring carbon atoms or a substituted or unsubstituted heterocyclic group having 5 to 50 ring atoms.

In an exemplary arrangement of the organic EL device of each exemplary embodiment, when the first emitting region at least includes the first emitting layer containing the first host material and the second emitting layer containing the second host material, R₃₀₁ to R₃₀₈ that are substituents of an anthracene skeleton in the compound represented by the formula (H1) are preferably hydrogen atoms in terms of preventing inhibition of intermolecular interaction and inhibiting decrease in electron mobility. However, R₃₀₁ to R₃₀₈ may be a substituted or unsubstituted aryl group having 6 to 50 ring carbon atoms or a substituted or unsubstituted heterocyclic group having 5 to 50 ring atoms.

An exemplary arrangement of the organic EL device of each exemplary embodiment may include the anode, the second emitting layer, the first emitting layer, and the cathode in this order. However, the order of the first emitting layer and the second emitting layer may be inversed.

For instance, in a case where the second emitting layer and the first emitting layer are layered in this order from a side close to the anode and the first host material contained in the first emitting layer is a compound represented by the formula (H1), the following phenomenon may occur. Therefore, R₃₀₁ to R₃₀₈ in the formula (H1) are each preferably not a bulky substituent.

Assuming that R₃₀₁ to R₃₀₈ in the formula (H1) are each a bulky substituent such as an alkyl group and a cycloalkyl group, intermolecular interaction may be inhibited to decrease the electron mobility relative to that of the second host material, so that the relationship of μe(H1)>μe(H2) shown by a later-described numerical formula (Numerical Formula 3) may not be satisfied. In a case where a compound represented by the formula (H1) is used as the first host material in the first emitting layer, it can be expected that satisfying the relationship of μe(H1)>μe(H2) inhibits a decrease in a recombination ability between holes and electrons in the second emitting layer and a decrease in luminous efficiency. It should be noted that substituents, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O) R₈₀₁, group represented by —COOR₈₀₂, halogen atom, cyano group, and nitro group are likely to be bulky, and an alkyl group and cycloalkyl group are likely to be further bulky.

In the compound represented by the formula (H1), R₃₀₁ to R₃₀₈, which are the substituents on the anthracene skeleton, are each preferably not a bulky substituent and preferably not an alkyl group and cycloalkyl group. More preferably, R₃₀₁ to R₃₀₈ are each not an alkyl group and cycloalkyl group. Still more preferably, R₃₀₁ to R₃₀₈ are each not an alkyl group, cycloalkyl group haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O) R₈₀₁, group represented by —COOR₈₀₂, halogen atom, cyano group, and nitro group.

In the formula (H1), examples of the substituent for the “substituted or unsubstituted” group on R₃₀₁ to R₃₀₈ also preferably do not include the above-described substituents that are likely to be bulky, especially a substituted or unsubstituted alkyl group and a substituted or unsubstituted cycloalkyl group. When examples of the substituent for the “substituted or unsubstituted” group on R₃₀₁ to R₃₀₈ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the compound described above is used as the first host material in the first emitting layer, a decrease in a recombination ability between holes and electrons in the second emitting layer and a decrease in the luminous efficiency can be inhibited.

Further preferably, R₃₀₁ to R₃₀₈ that are the substituents on the anthracene skeleton are not bulky substituents and R₃₀₁ to R₃₀₈ as substituents are unsubstituted. Assuming that R₃₀₁ to R₃₀₈ that are the substituents on the anthracene skeleton are not bulky substituents and substituents are bonded to R₃₀₁ to R₃₀₈ that are not bulky substituents, the substituents bonded to R₃₀₁ to R₃₀₈ are preferably not bulky substituents; and the substituents bonded to R₂₀₁ to R₂₀₈ serving as substituents are preferably not an alkyl group and cycloalkyl group, and more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O) R₈₀₁, group represented by —COOR₈₀₂, halogen atom, cyano group, and nitro group.

In the first host material, the groups specified to be “substituted or unsubstituted” are each preferably an “unsubstituted” group.

In an exemplary arrangement of the organic EL device of each exemplary embodiment, the emitting layer contains a compound represented by the formula (H10) as the first host material. The compound represented by the formula (H10) has or does not have at least one deuterium atom.

In an exemplary arrangement of the organic EL device of each exemplary embodiment, the emitting layer contains a compound represented by the formula (H20) as the first host material. The compound represented by the formula (H20) has or does not have at least one deuterium atom.

In an exemplary arrangement of the organic EL device of each exemplary embodiment, the emitting layer contains, as the first host material, the compound represented by the formula (H10) and the compound represented by the formula (H20). In this arrangement, at least one of the compound represented by the formula (H10) or the compound represented by the formula (H20) preferably has at least one deuterium atom.

In an exemplary arrangement of the organic EL device of each exemplary embodiment, when the emitting layer contains the compound represented by the formula (H10) and the compound represented by the formula (H20) as the first host material, the compound represented by the formula (H10) does not substantially contain a deuterium atom and the compound represented by the formula (H20) contains at least one deuterium atom.

In an exemplary arrangement of the organic EL device of each exemplary embodiment, when the emitting layer contains the compound represented by the formula (H10) and the compound represented by the formula (H20) as the first host material, the compound represented by the formula (H20) does not substantially contain a deuterium atom and the compound represented by the formula (H10) does not contain at least one deuterium atom.

Here, the phrase that “the compound does not substantially contain a deuterium atom” means that the compound does not contain a deuterium atom at all or the compound is allowed to contain a deuterium atom in a natural abundance. The natural abundance ratio of deuterium atoms is, for example, 0.015% or less.

### Production Method of First Host Material

The first host material can be produced by a known method. The first host material can also be produced based on a known method through a known alternative reaction using a known material(s) tailored for the target compound.

### Specific Examples of First Host Material

Specific examples of the first host material include the following compounds. It should however be noted that the invention is not limited to the specific examples of the first host material.

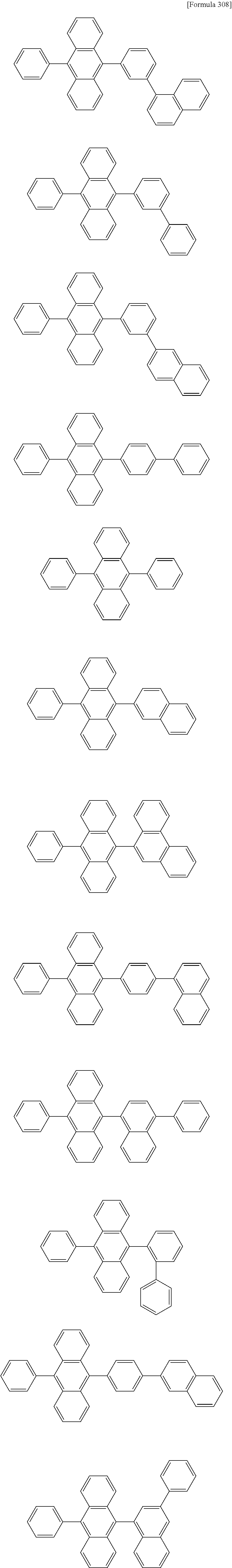

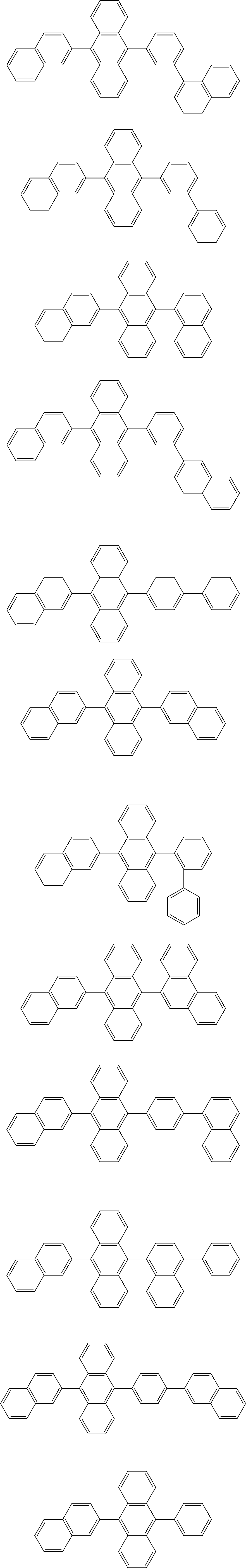

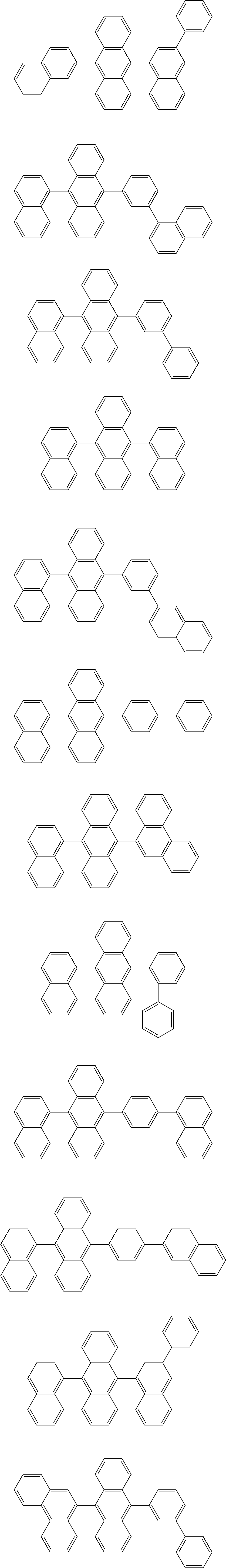

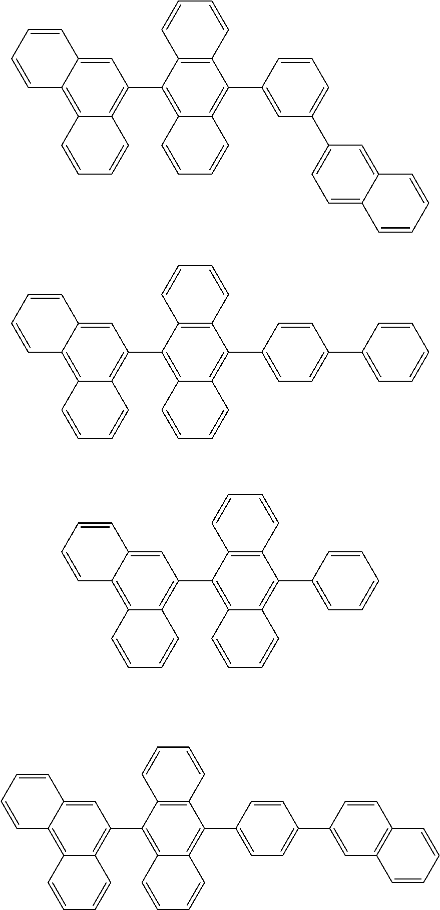

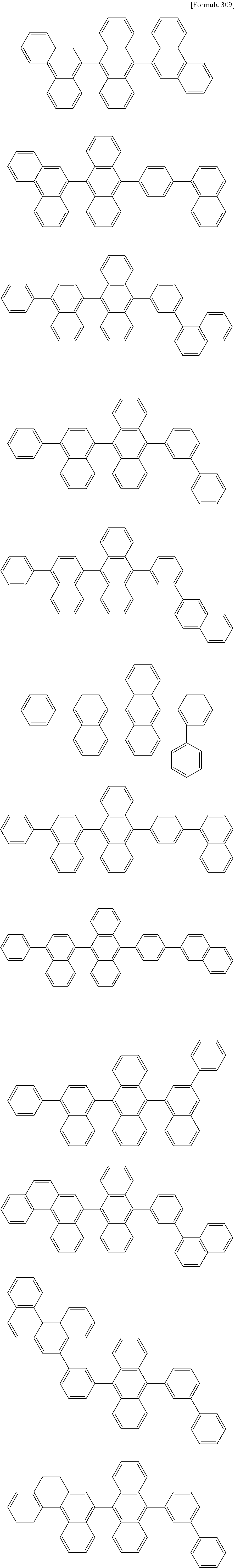

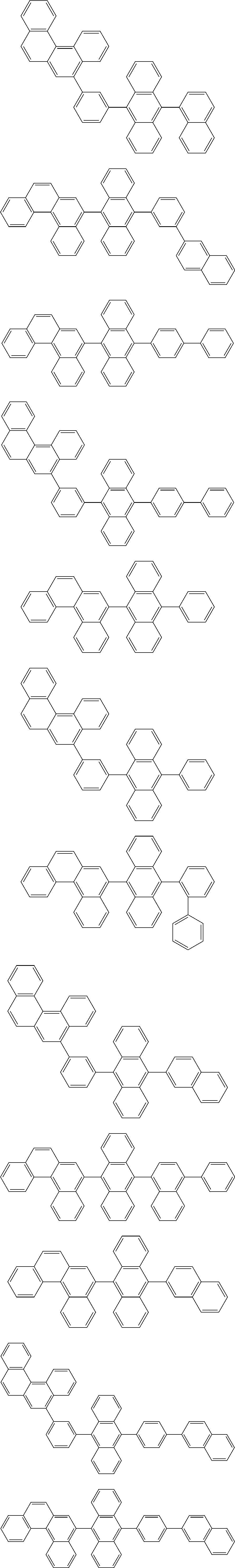

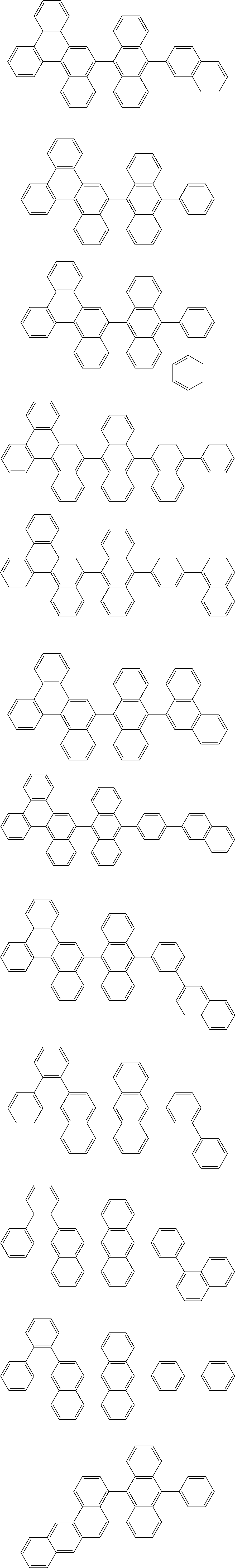

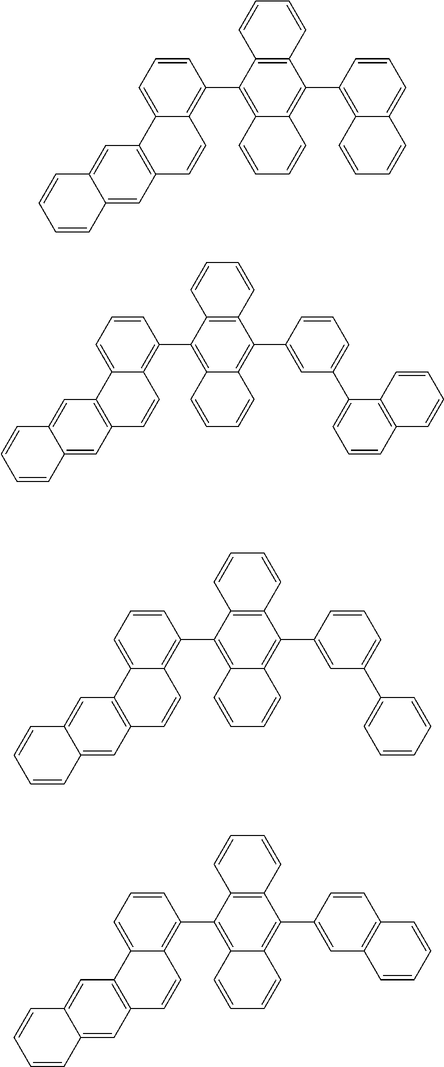

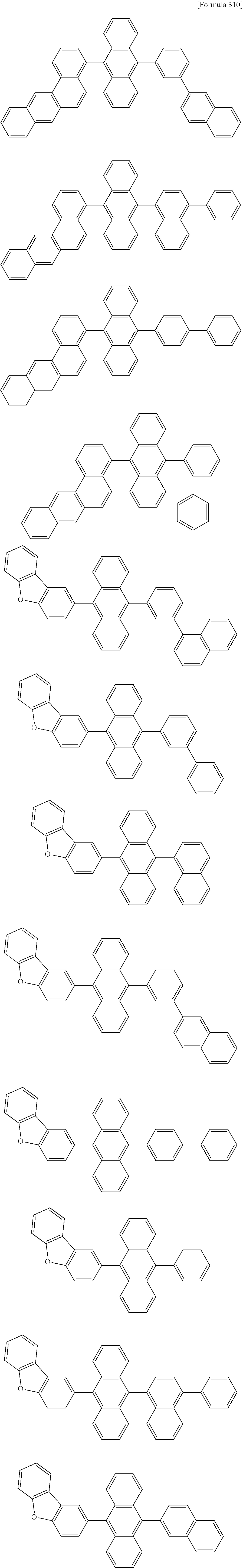

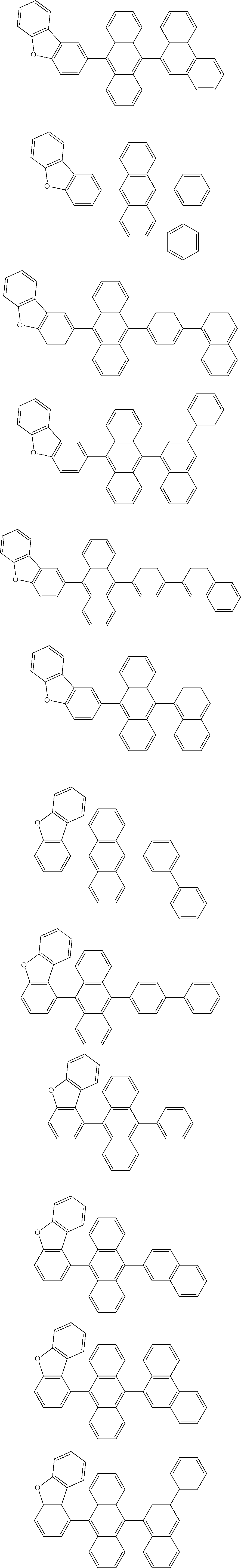

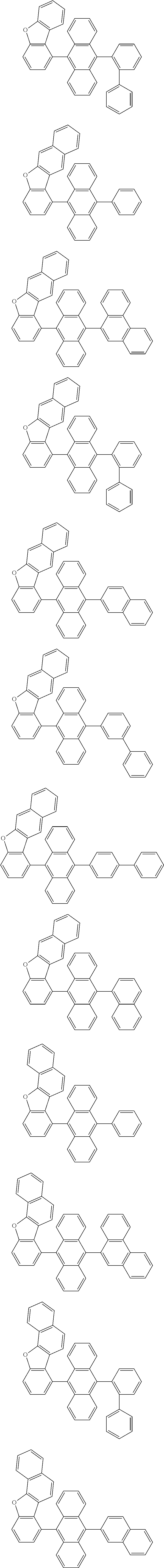

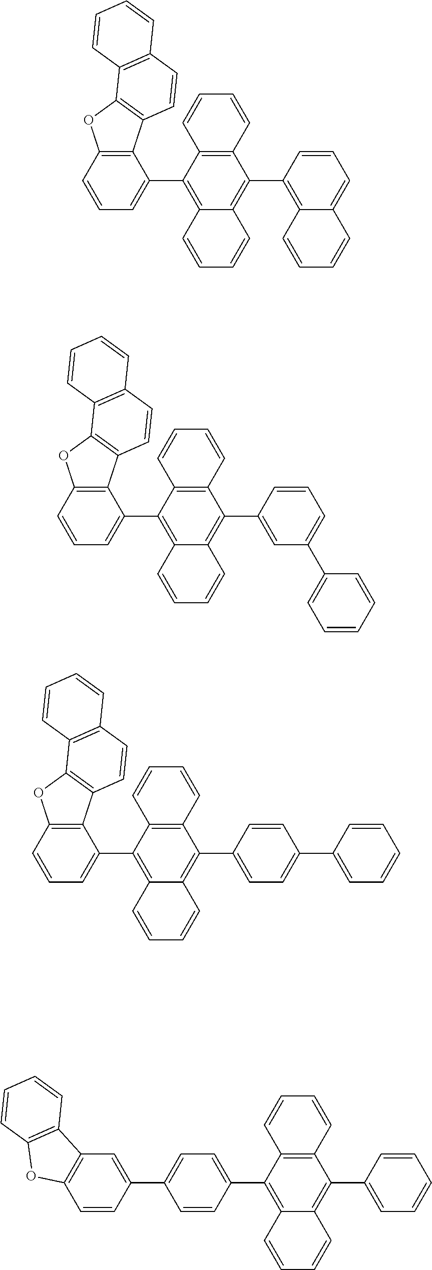 [SMILES: c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1 ; c1ccc(-c2c3ccccc3c(-c3ccc(-c4ccc5oc6ccccc6c5c4)cc3)c3ccccc23)cc1]

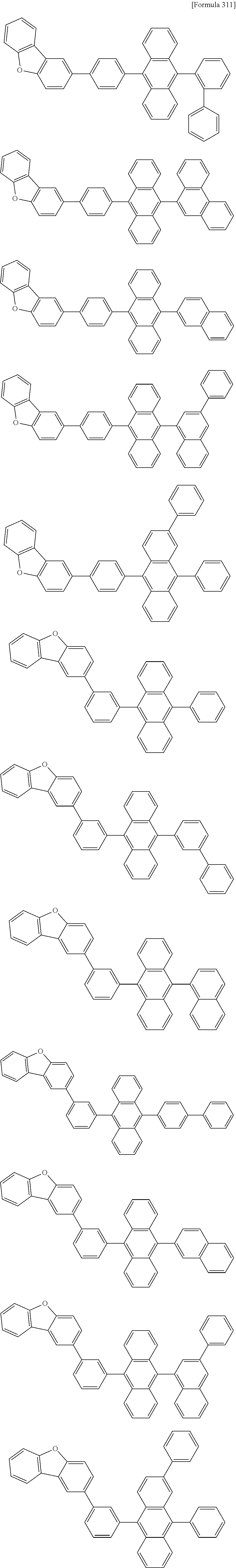

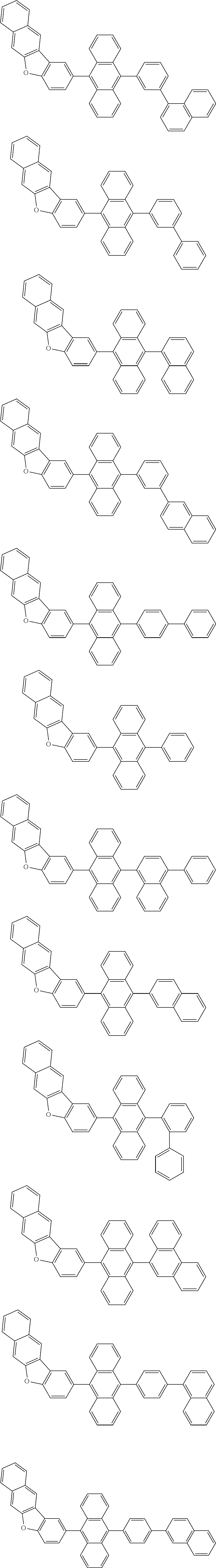

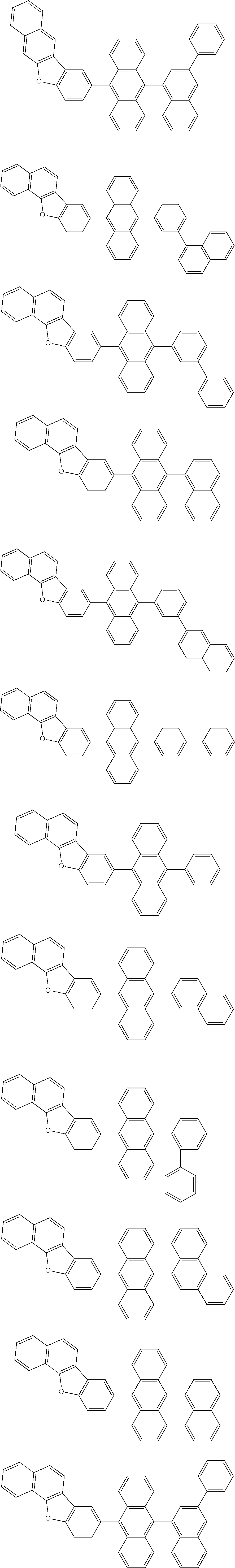

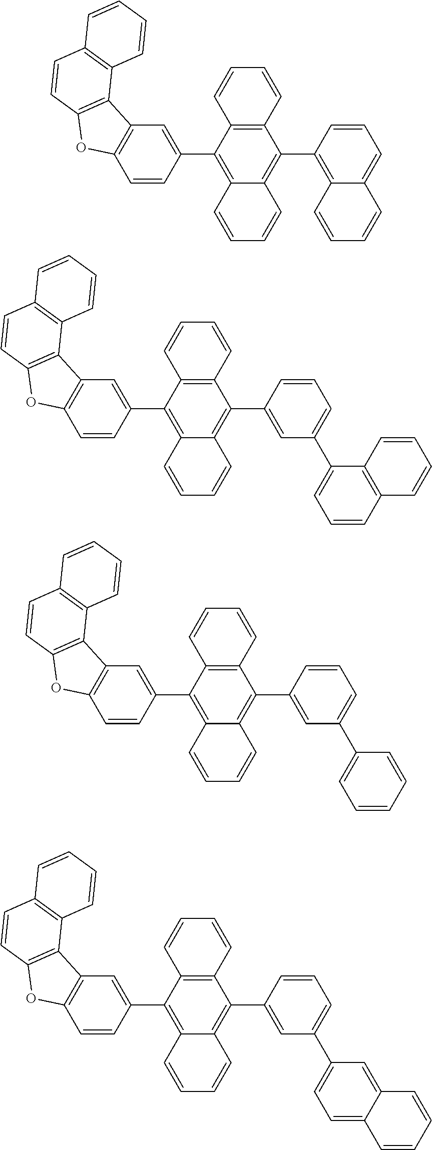 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

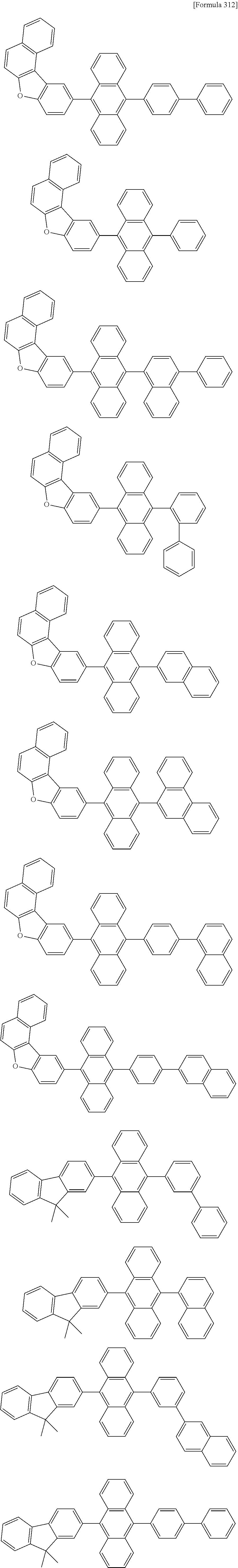

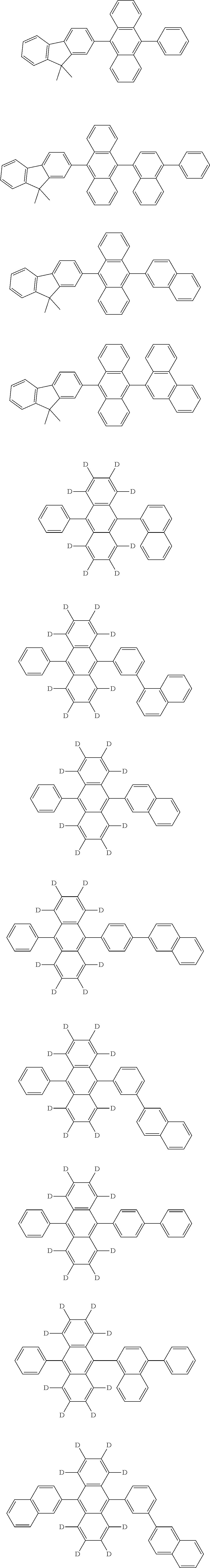

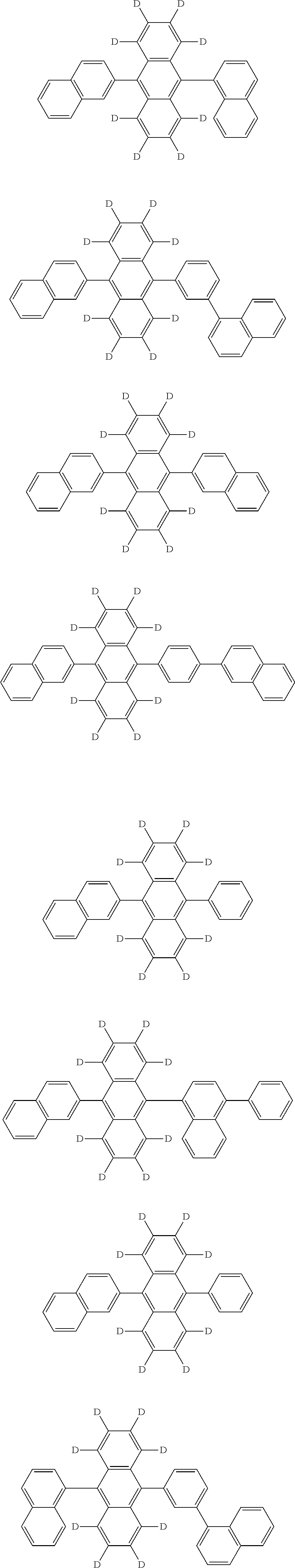

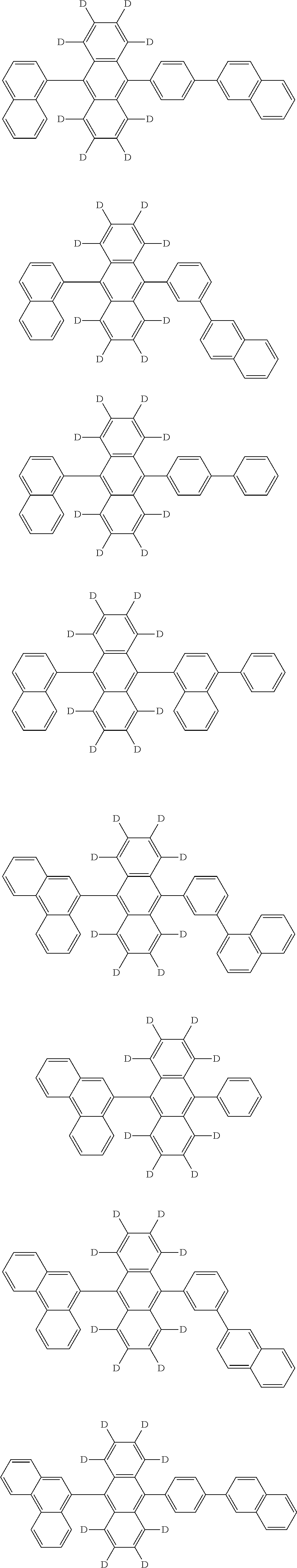

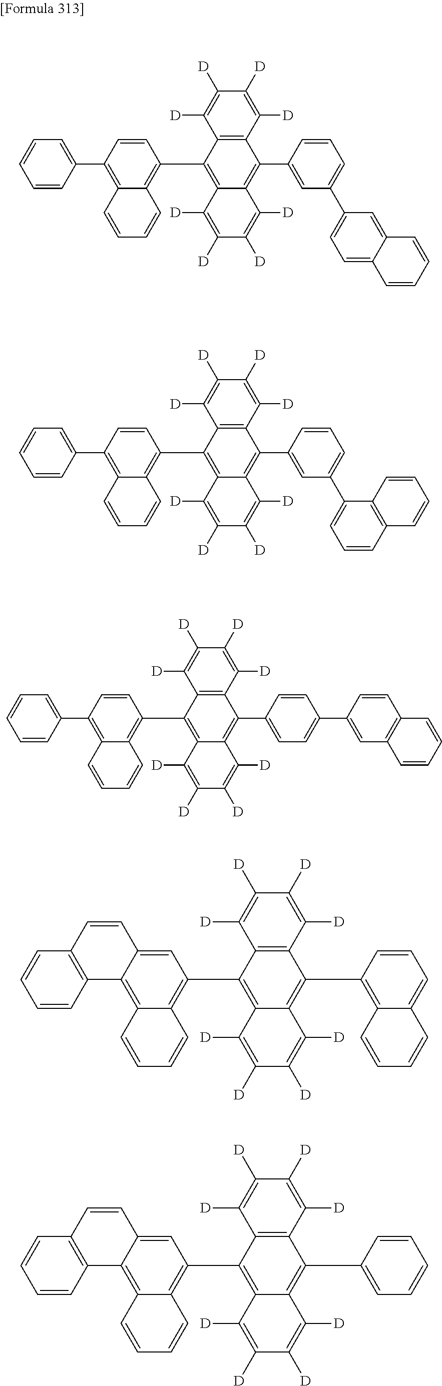

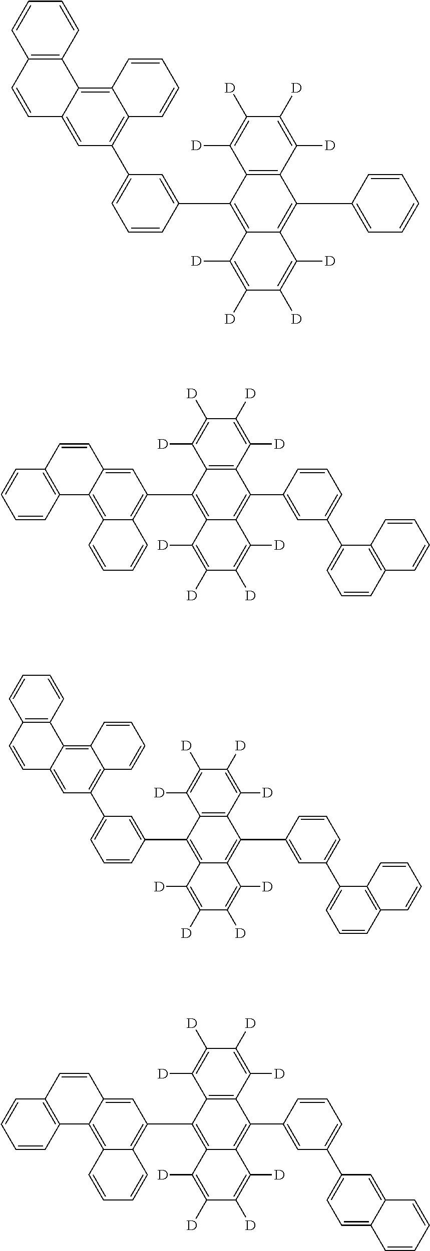 [SMILES: [2H]c1c([2H])c([2H])c2c(-c3cccc(-c4cc5ccc6ccccc6c5c5ccccc45)c3)c3c([2H])c([2H])c([2H])c([2H])c3c(-c3ccccc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3cccc(-c4cccc5ccccc45)c3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4ccc5ccccc5c4)c3)c2c1[2H]]

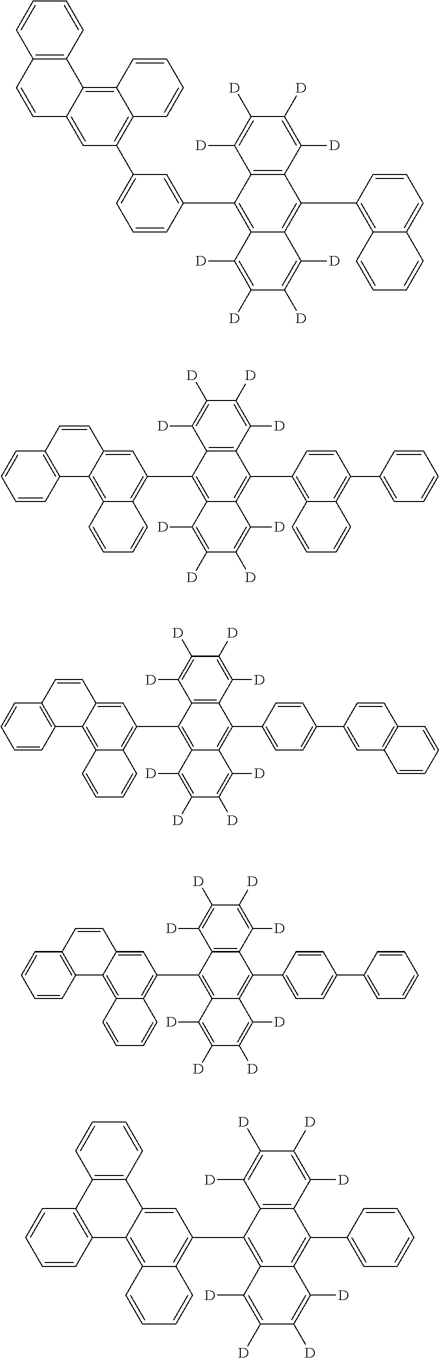

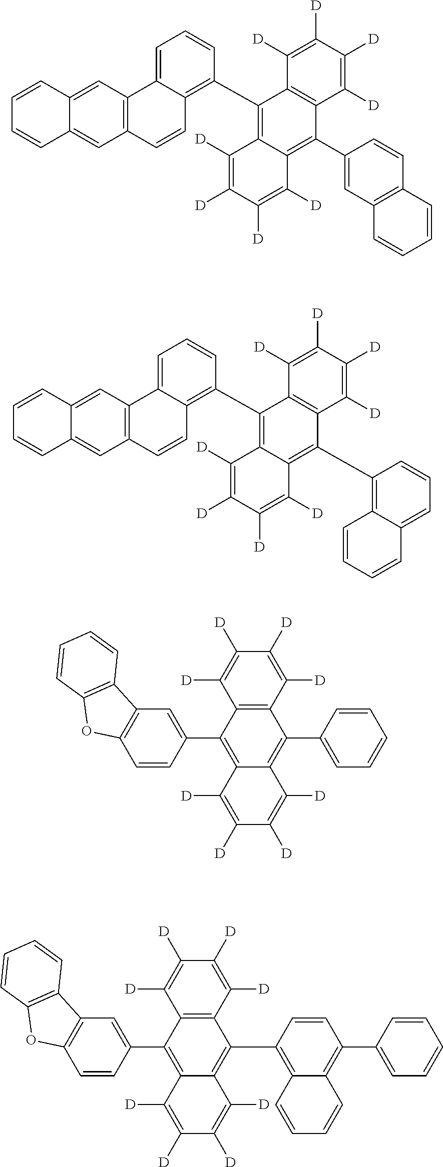 [SMILES: [2H]c1c([2H])c([2H])c2c(-c3cccc4c3ccc3cc5ccccc5cc34)c3c([2H])c([2H])c([2H])c([2H])c3c(-c3ccc4ccccc4c3)c2c1[2H] ; [2H]c1c([2H])c([2H])c2c(-c3cccc4c3ccc3cc5ccccc5cc34)c3c([2H])c([2H])c([2H])c([2H])c3c(-c3cccc4ccccc34)c2c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4oc5ccccc5c4c3)c2c1[2H]]

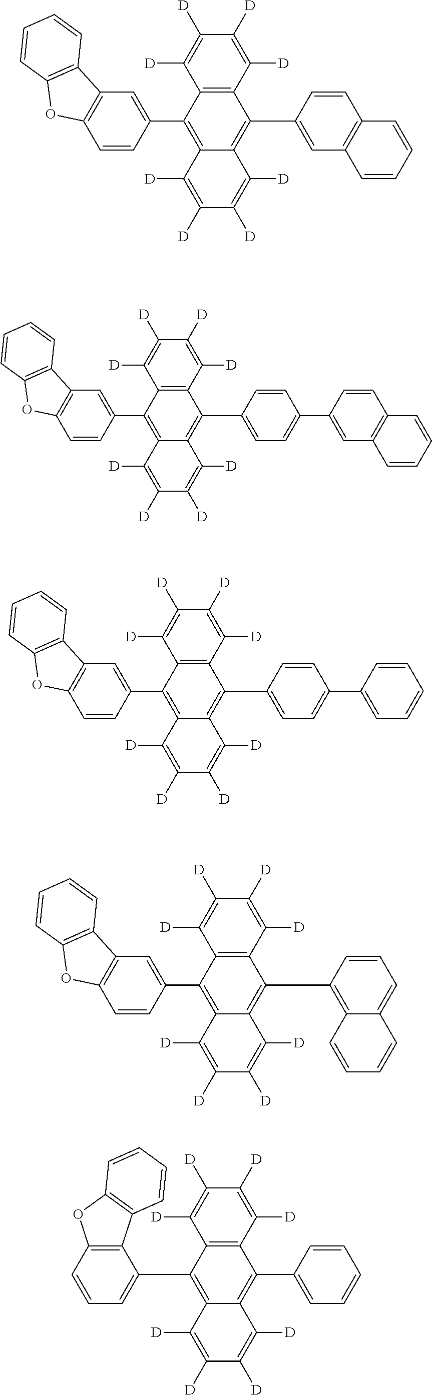

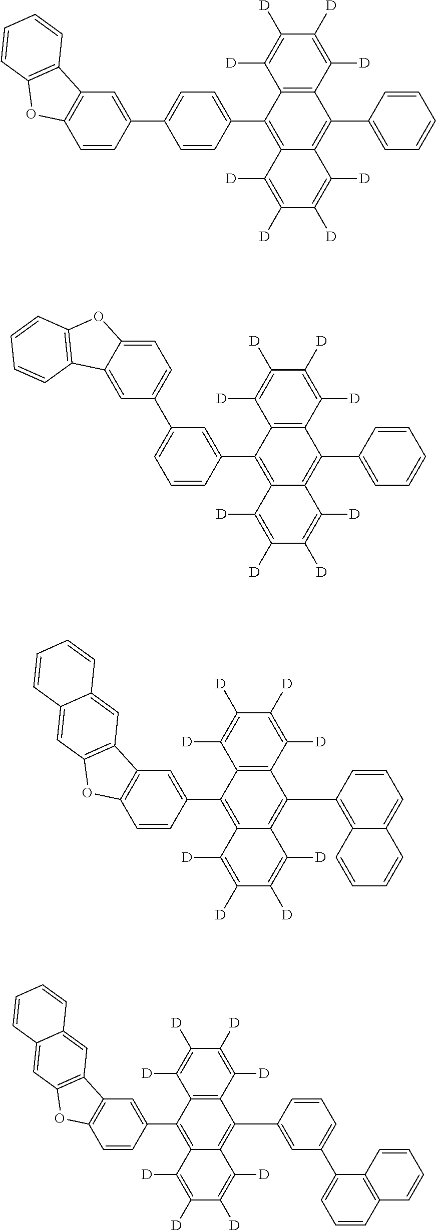 [SMILES: [2H]c1c([2H])c([2H])c2c(-c3ccc(-c4ccc5oc6ccccc6c5c4)cc3)c3c([2H])c([2H])c([2H])c([2H])c3c(-c3ccccc3)c2c1[2H] ; [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H]]

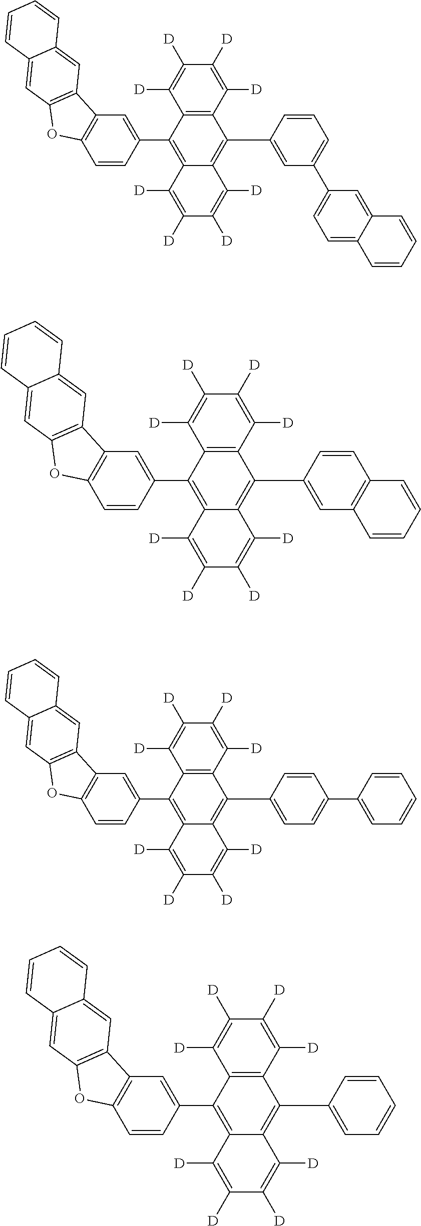 [SMILES: [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H]]

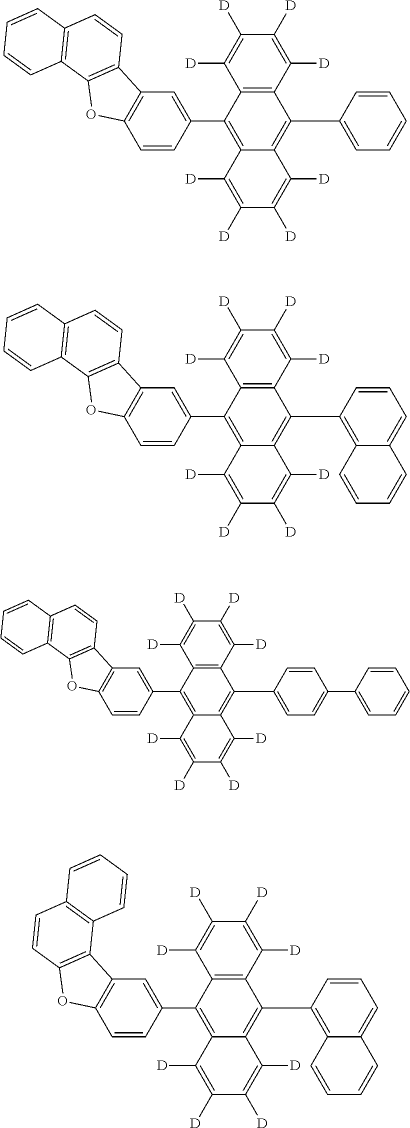 [SMILES: [2H]c1c([2H])c([2H])c2c(-c3ccc4oc5c6ccccc6ccc5c4c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H]]

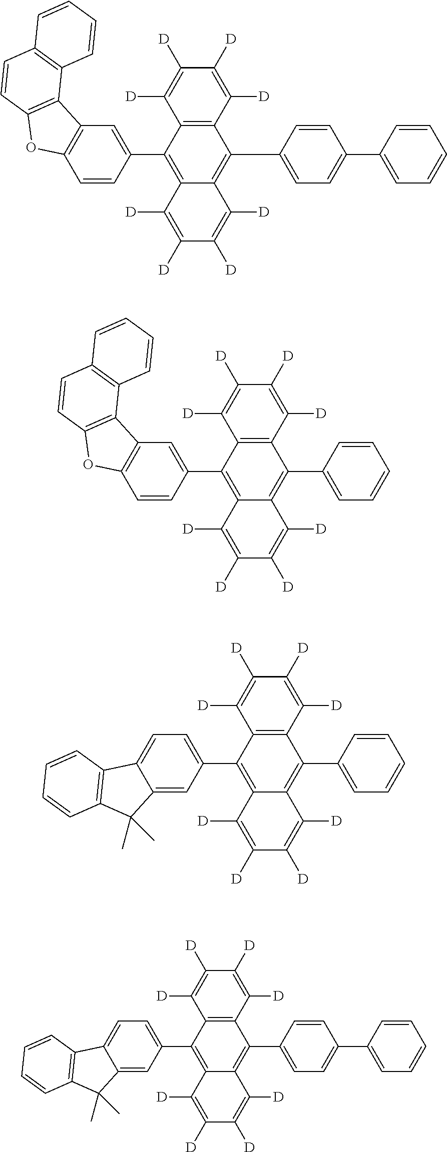 [SMILES: [2H]c1c([2H])c([2H])c2c(-c3ccc4oc5ccc6ccccc6c5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5ccc6ccccc6c5c4c3)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccccc4)cc3)c2c1[2H]]

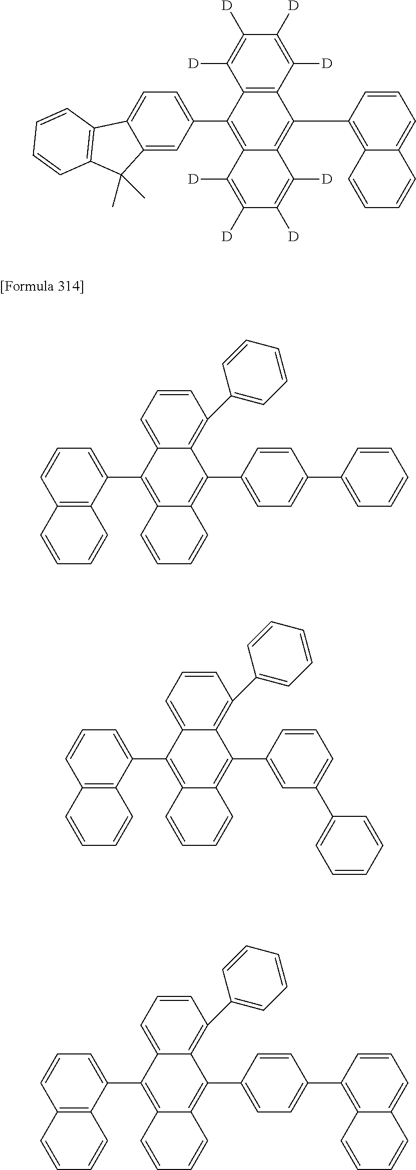

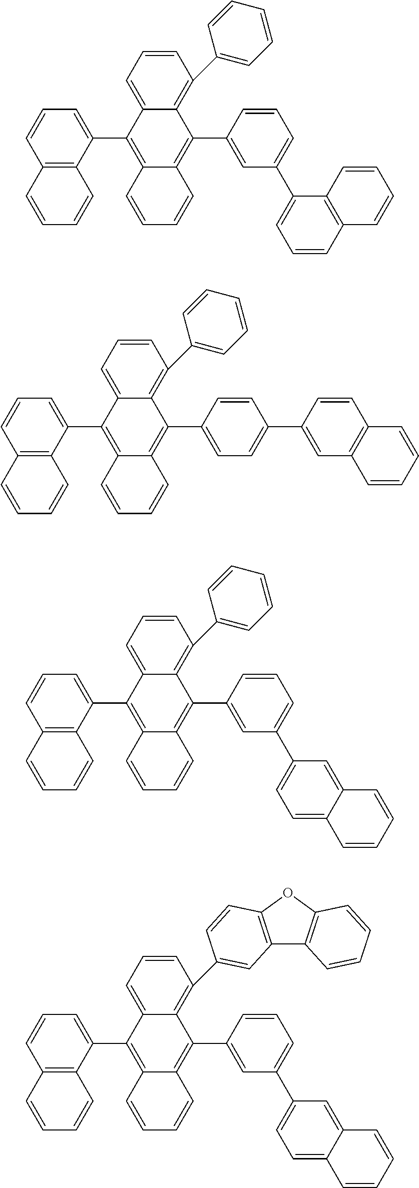

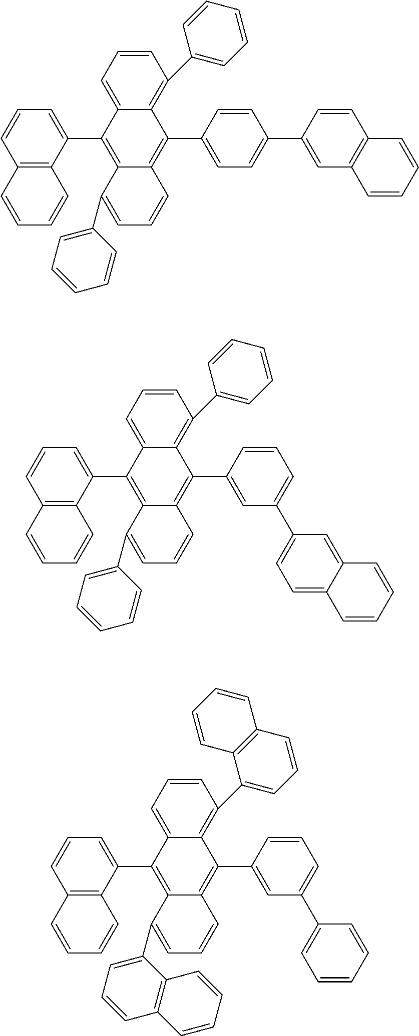

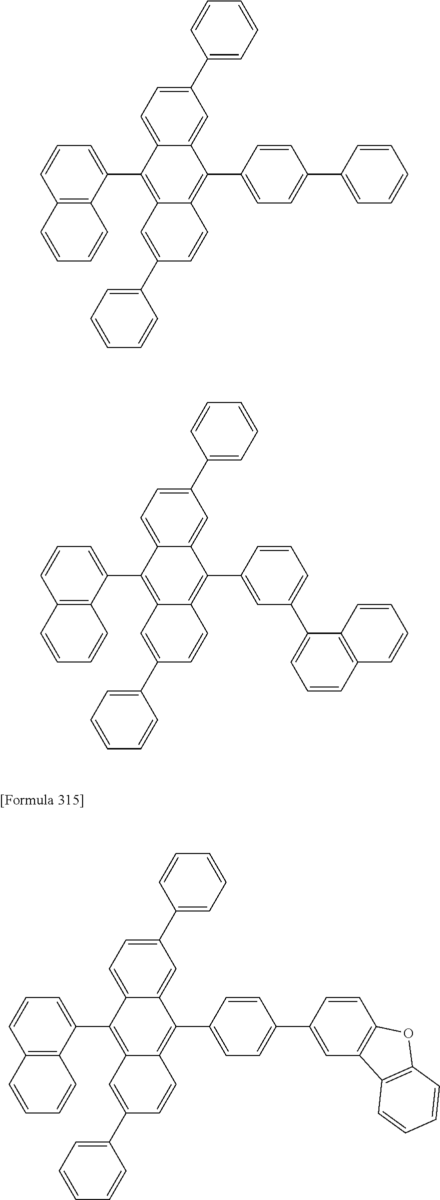

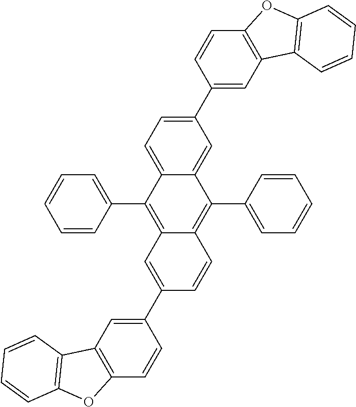 [SMILES: c1ccc(-c2c3ccc(-c4ccc5oc6ccccc6c5c4)cc3c(-c3ccccc3)c3ccc(-c4ccc5oc6ccccc6c5c4)cc23)cc1]

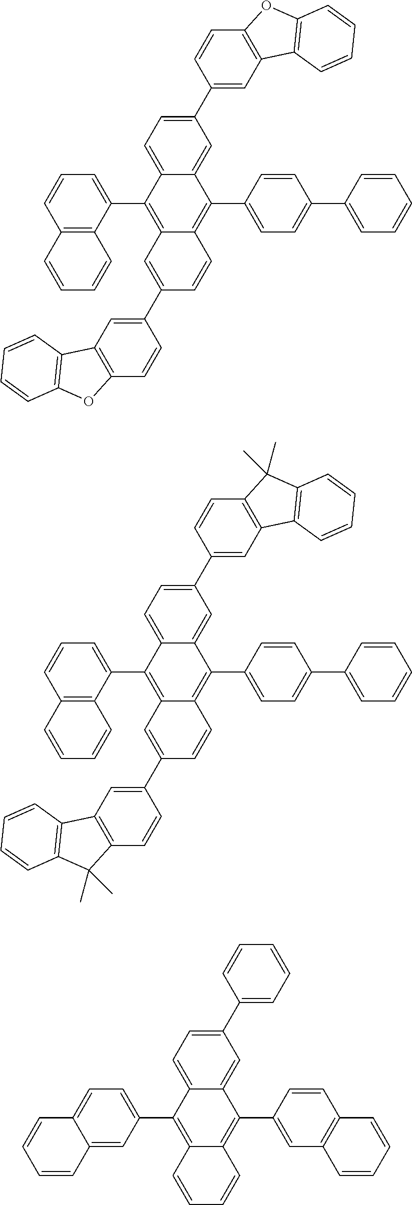

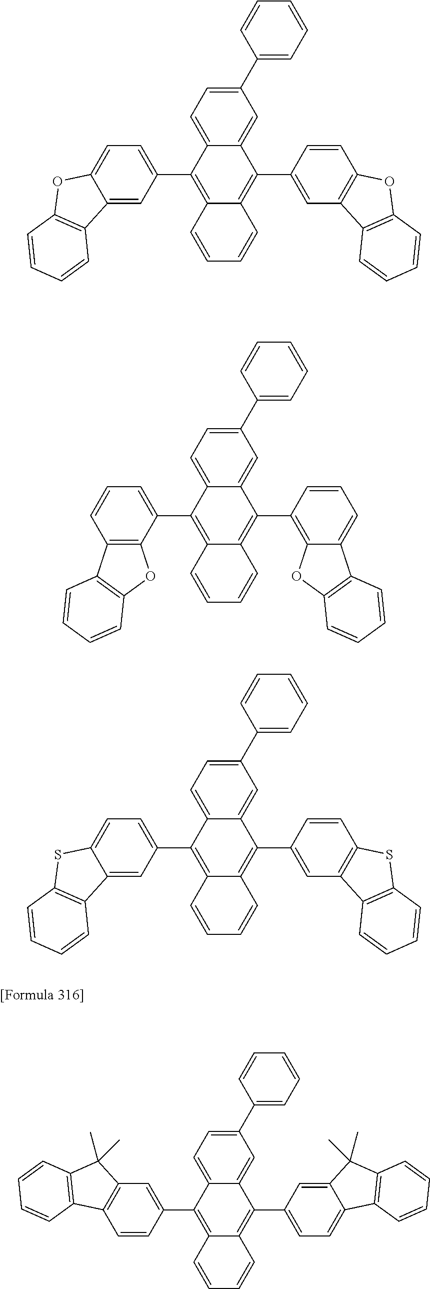

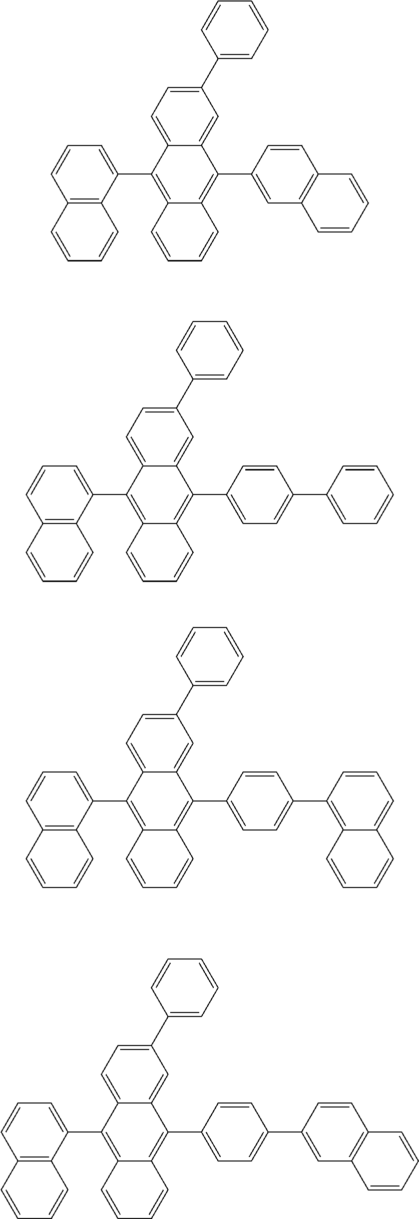

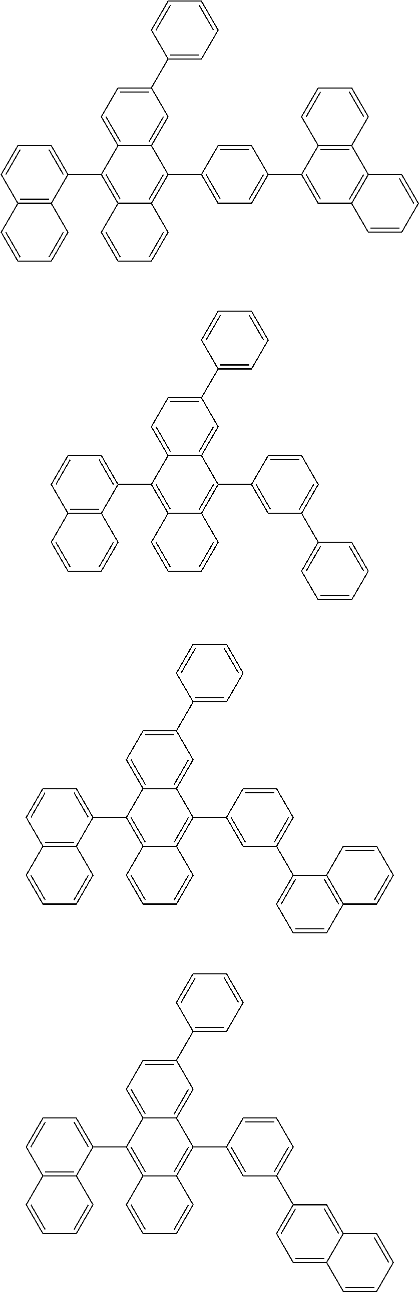

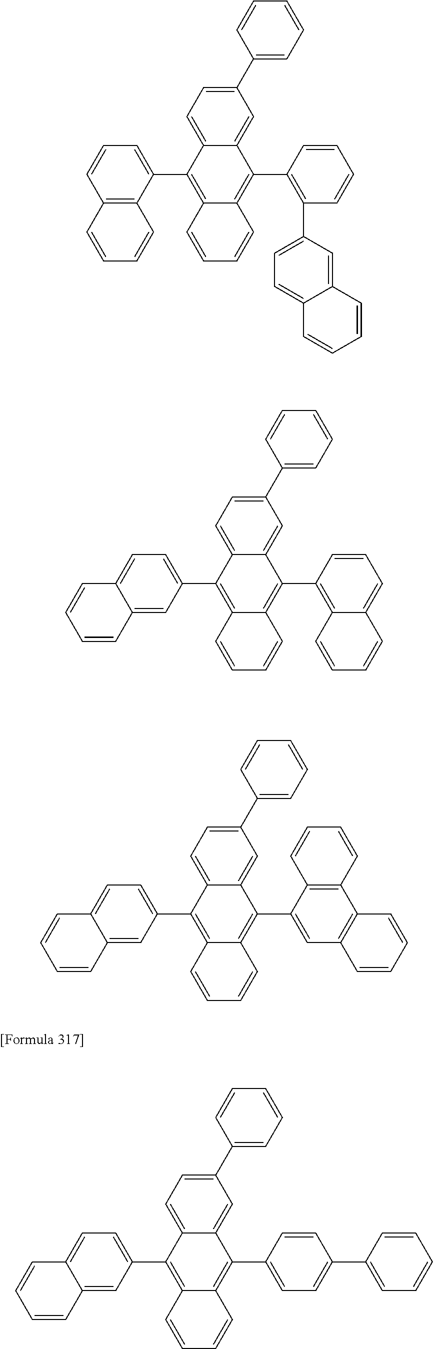

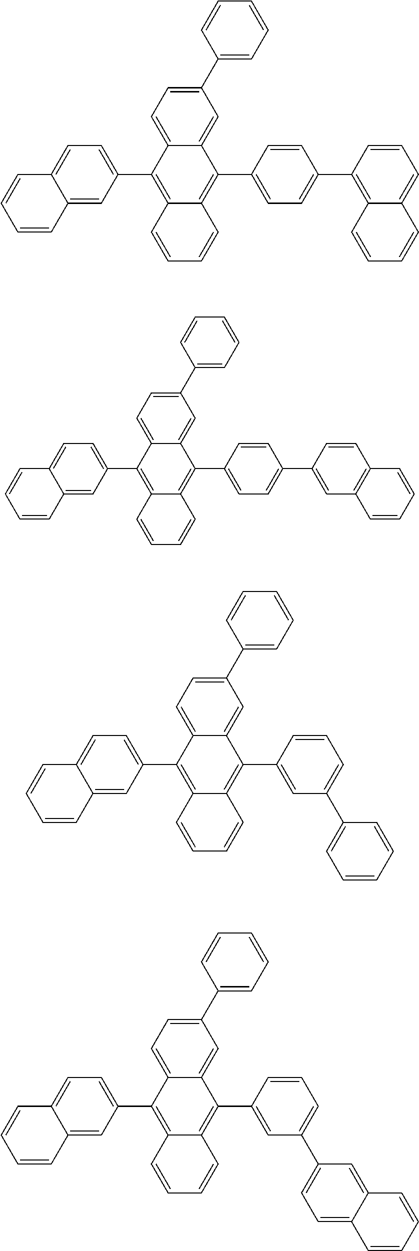

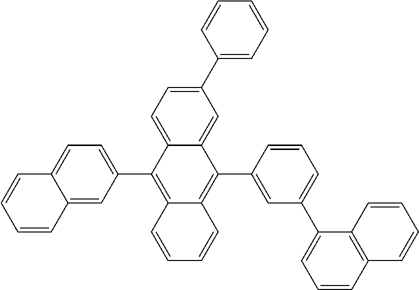 [SMILES: c1ccc(-c2ccc3c(-c4ccc5ccccc5c4)c4ccccc4c(-c4cccc(-c5cccc6ccccc56)c4)c3c2)cc1]

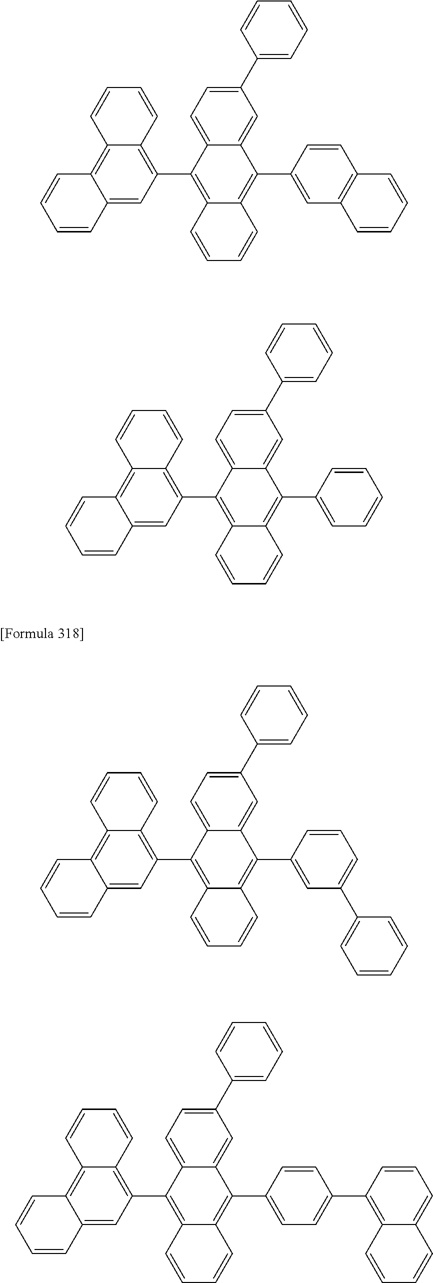

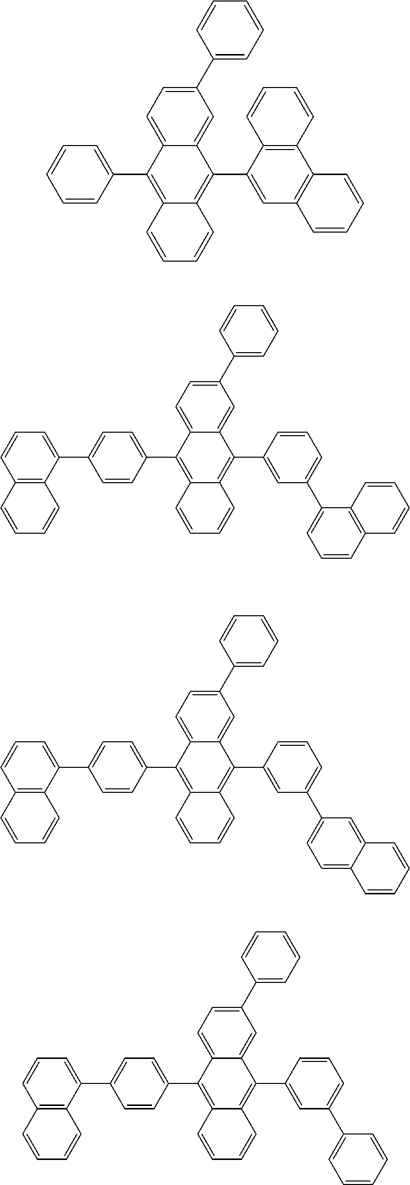

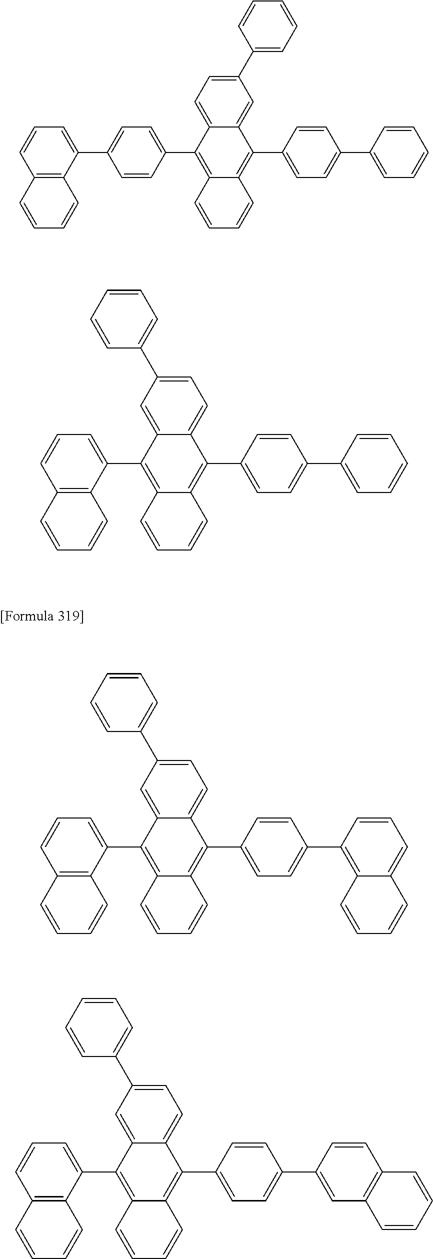

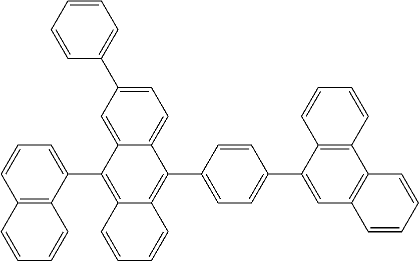 [SMILES: c1ccc(-c2ccc3c(-c4ccc(-c5cc6ccccc6c6ccccc56)cc4)c4ccccc4c(-c4cccc5ccccc45)c3c2)cc1]

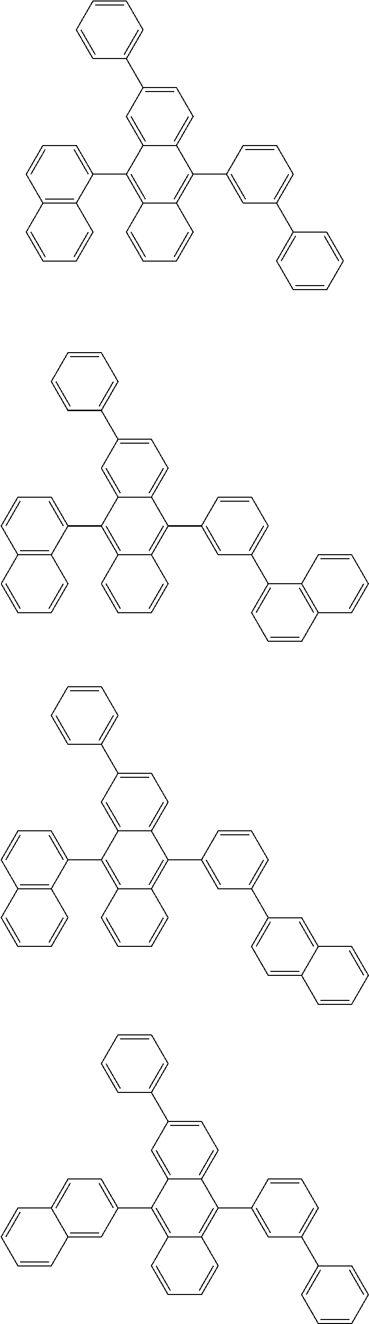

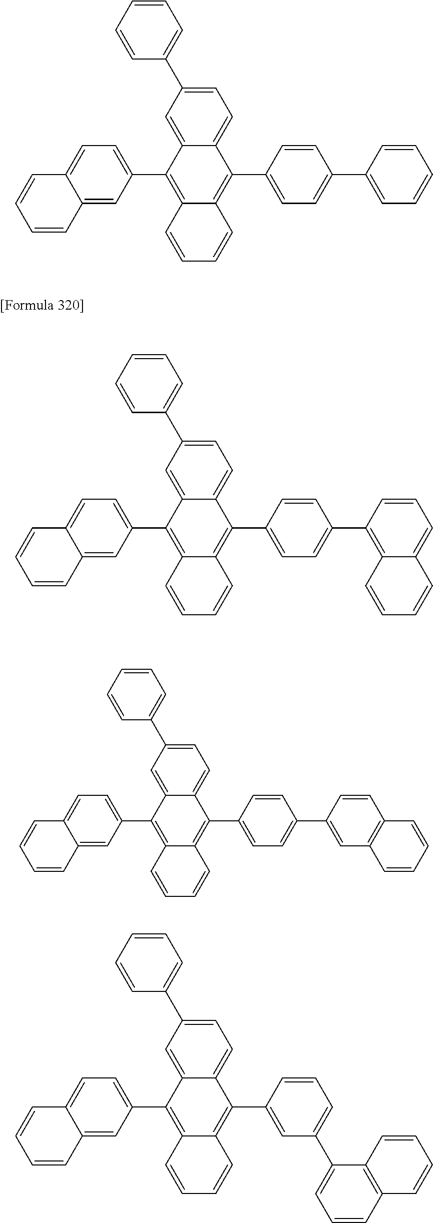

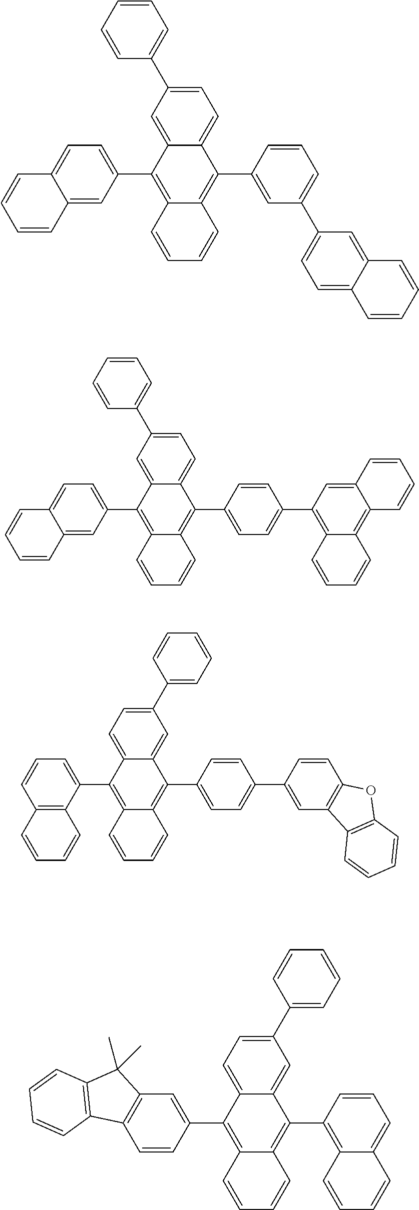

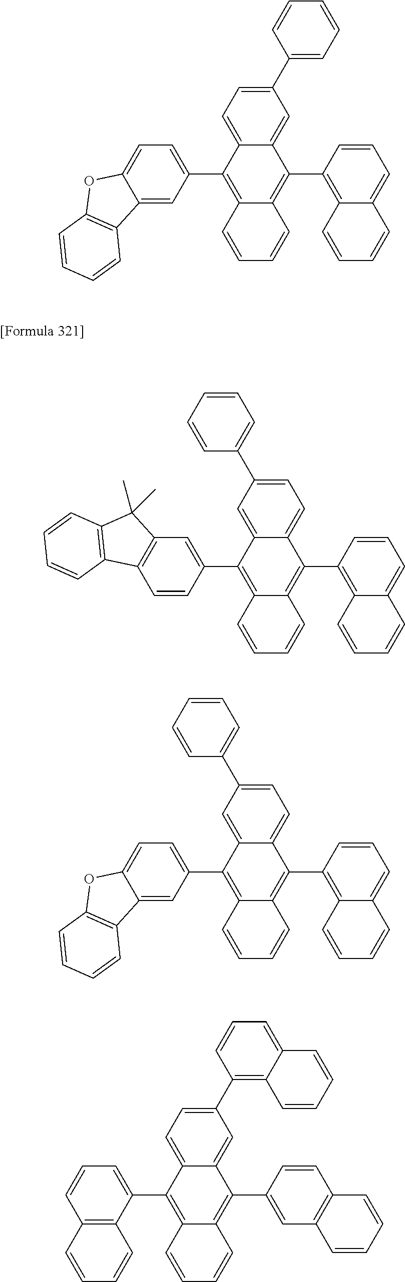

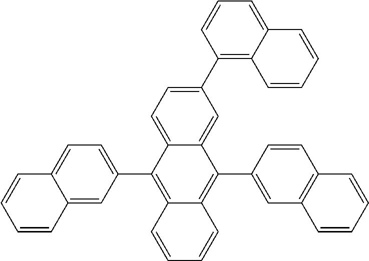 [SMILES: c1ccc2cc(-c3c4ccccc4c(-c4ccc5ccccc5c4)c4cc(-c5cccc6ccccc56)ccc34)ccc2c1]

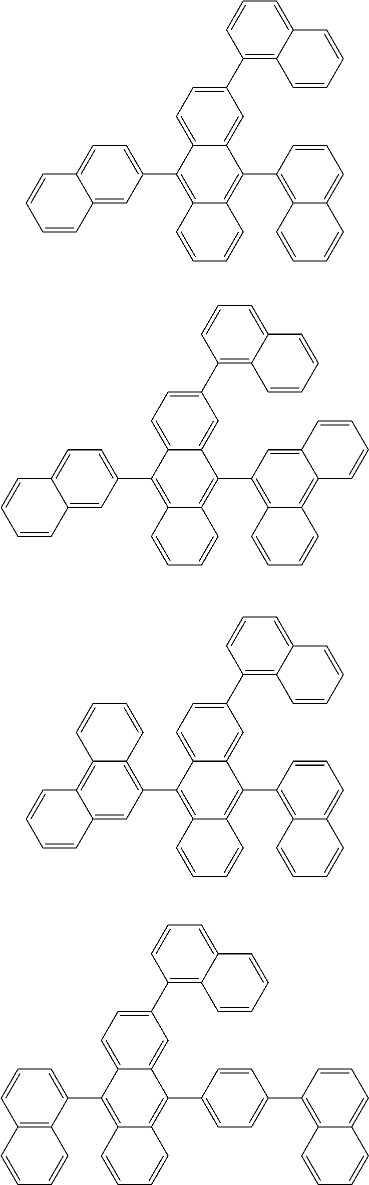

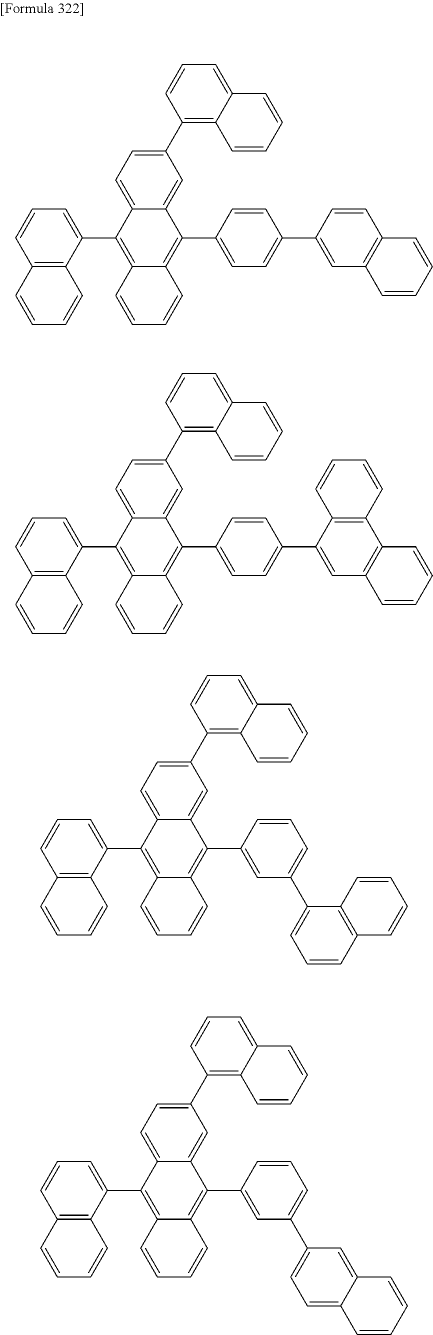

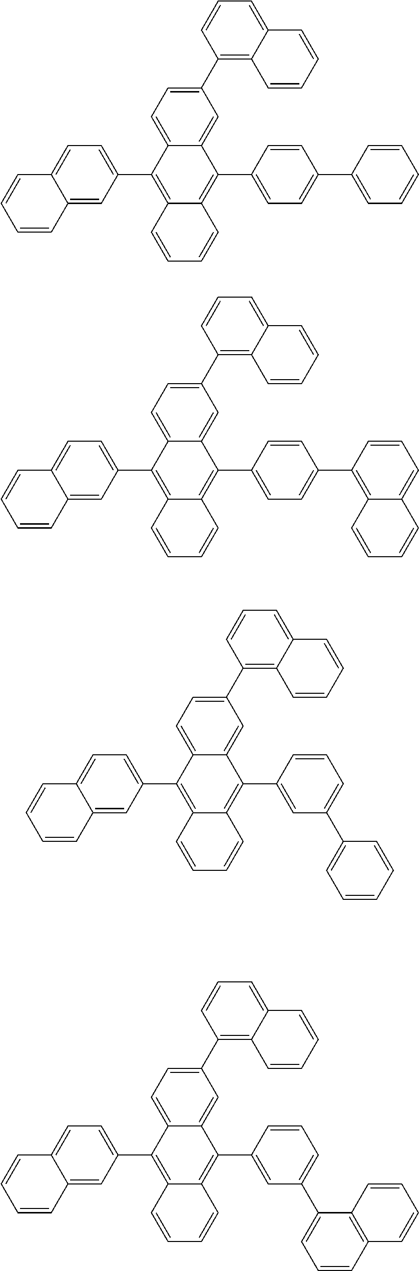

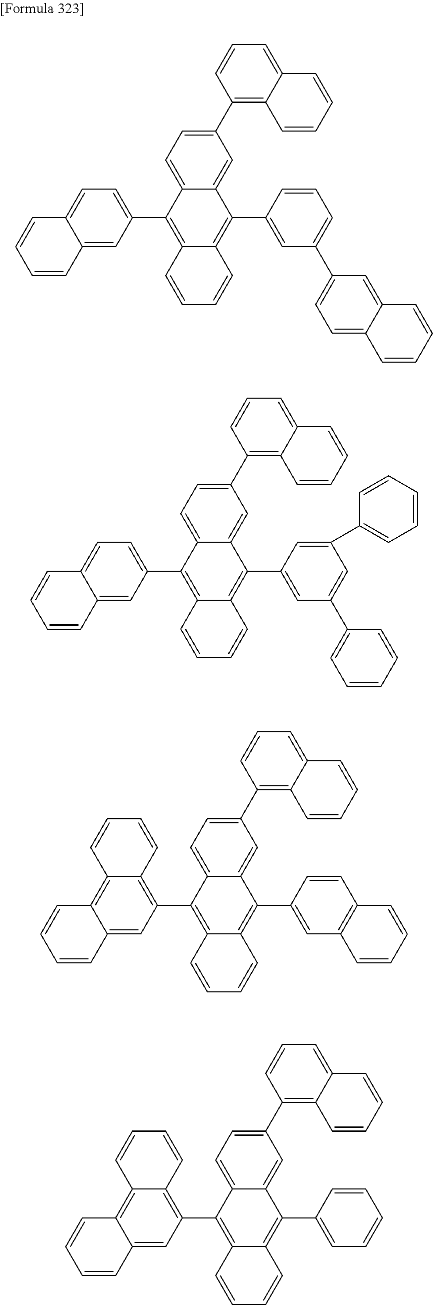

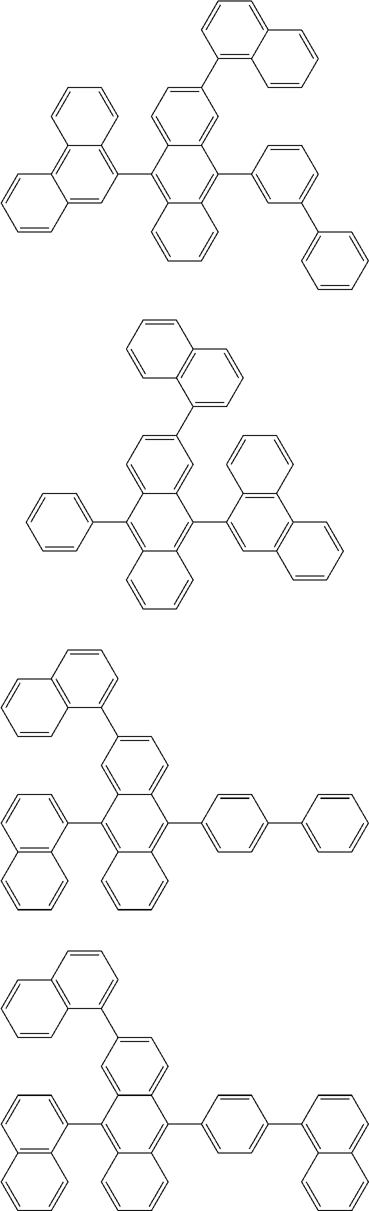

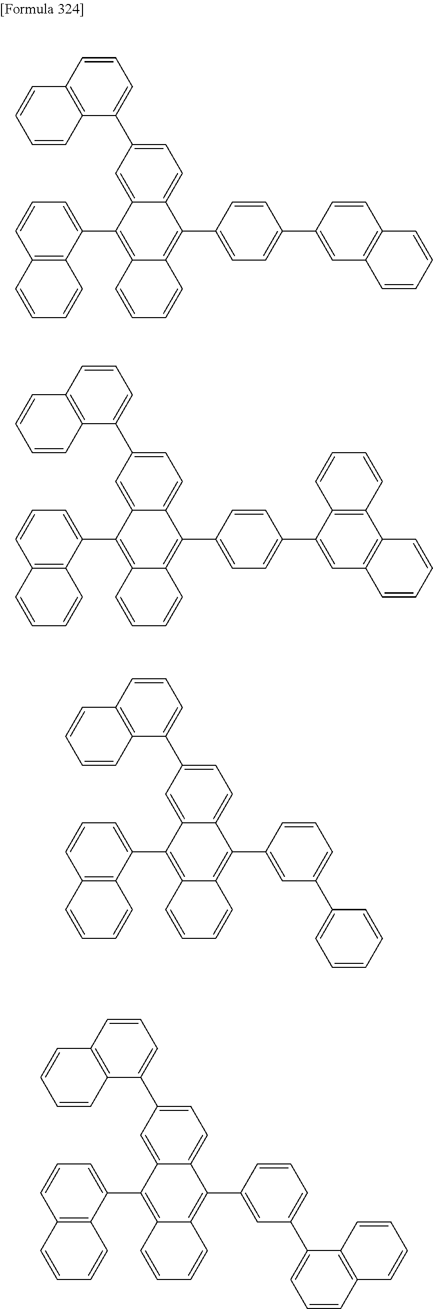

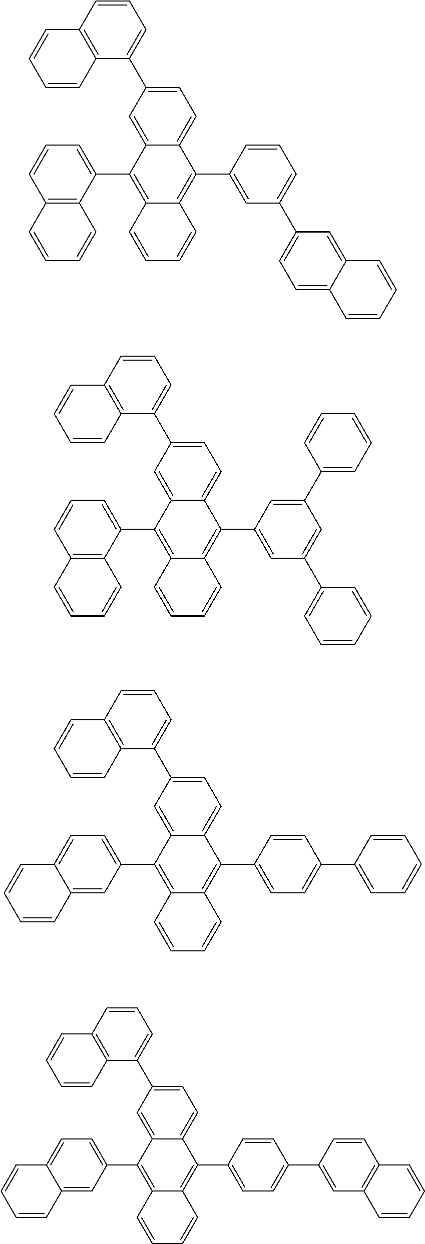

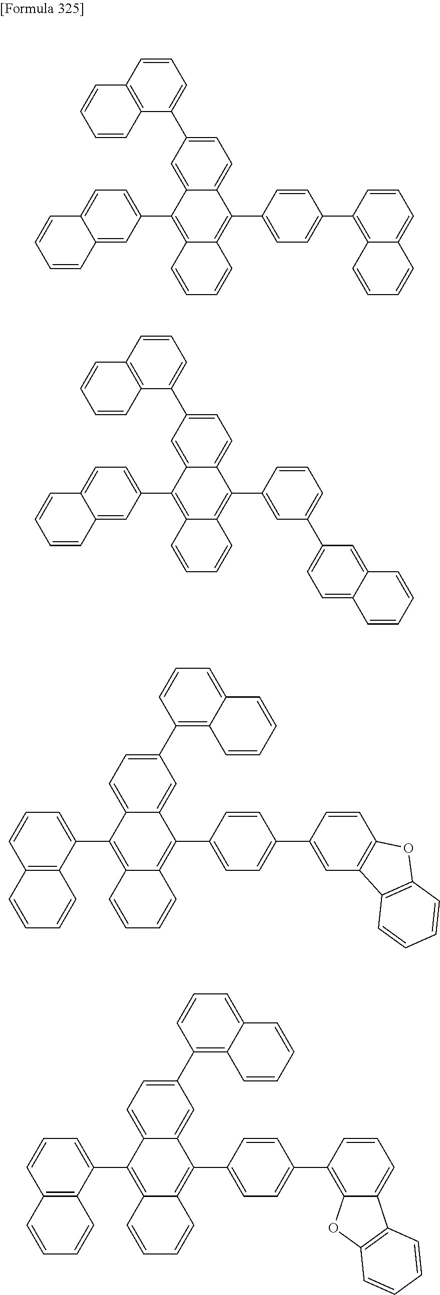

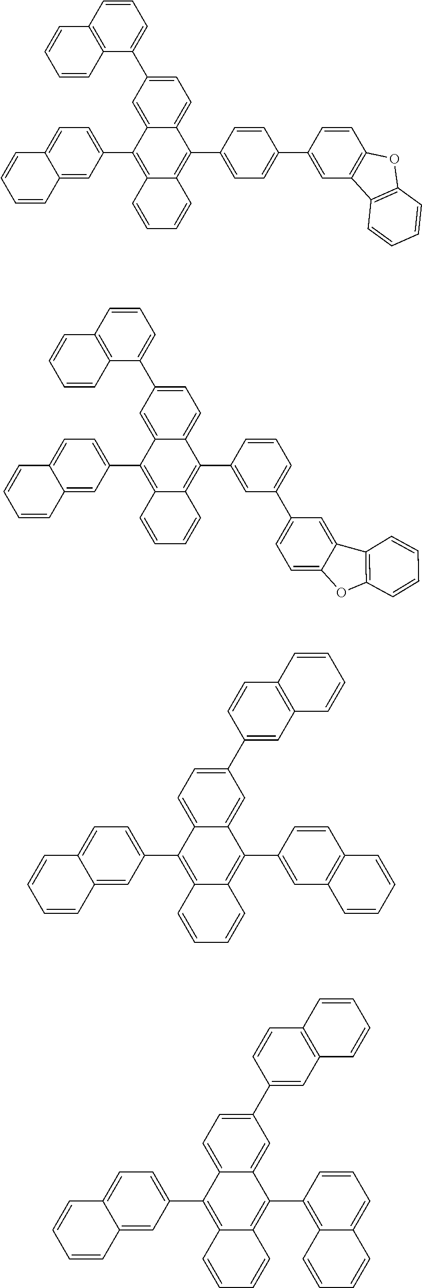

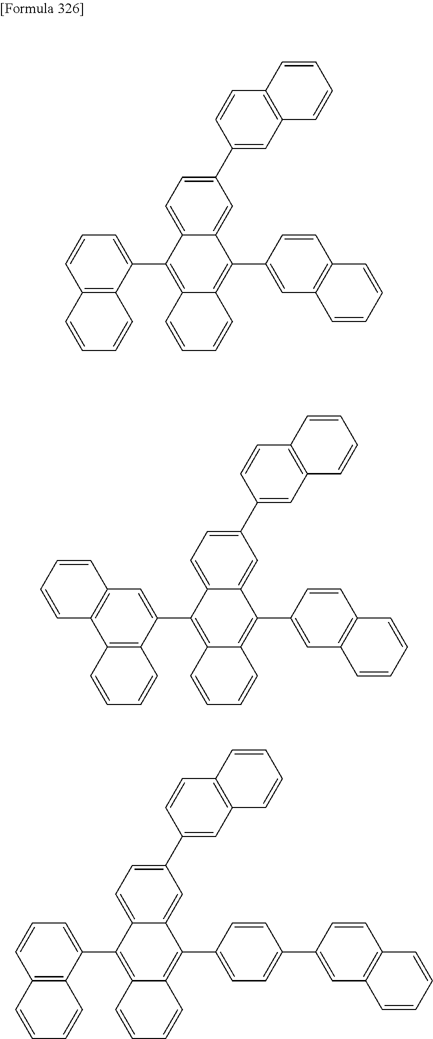

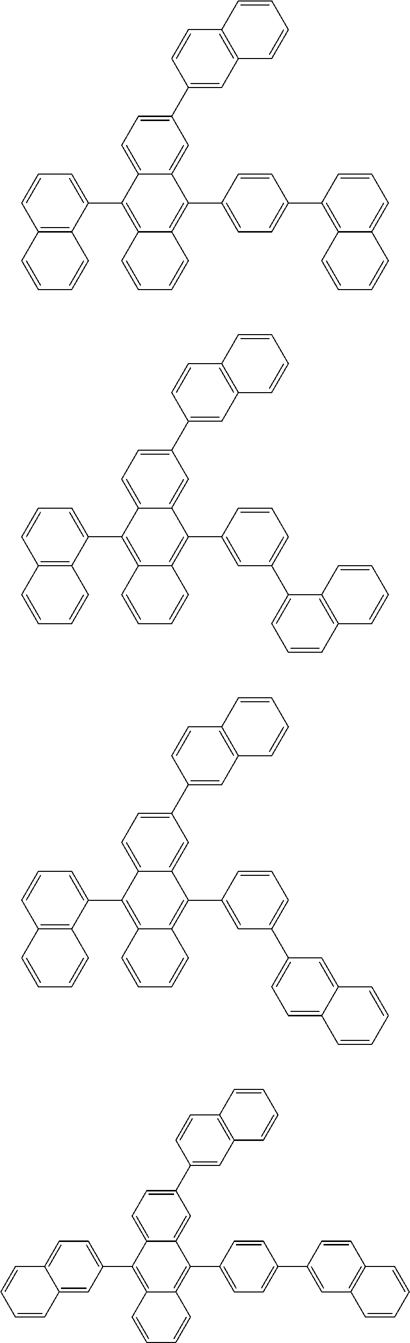

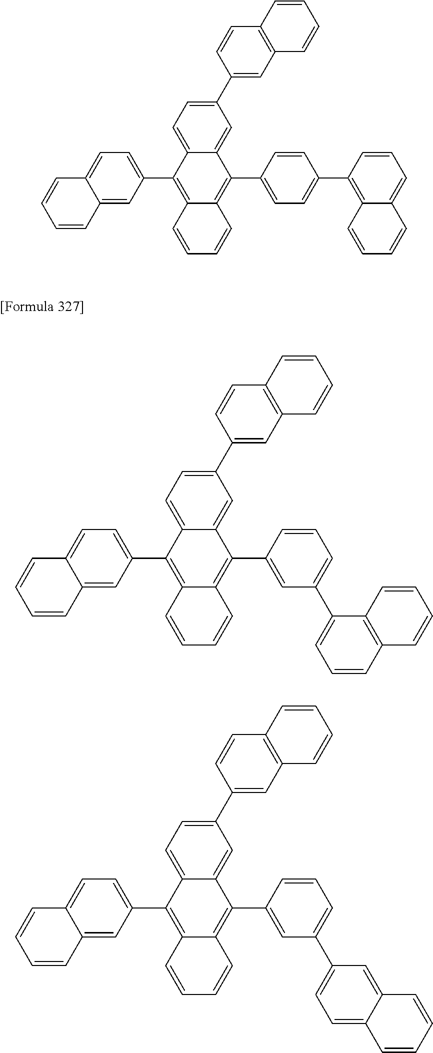

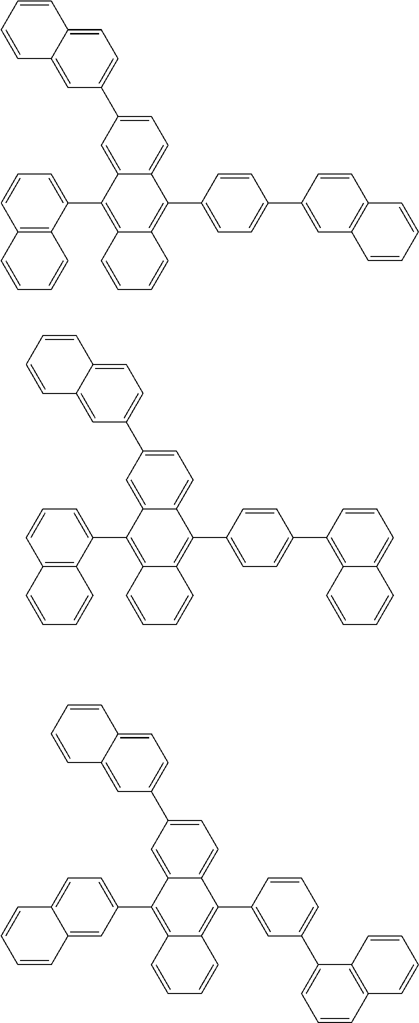

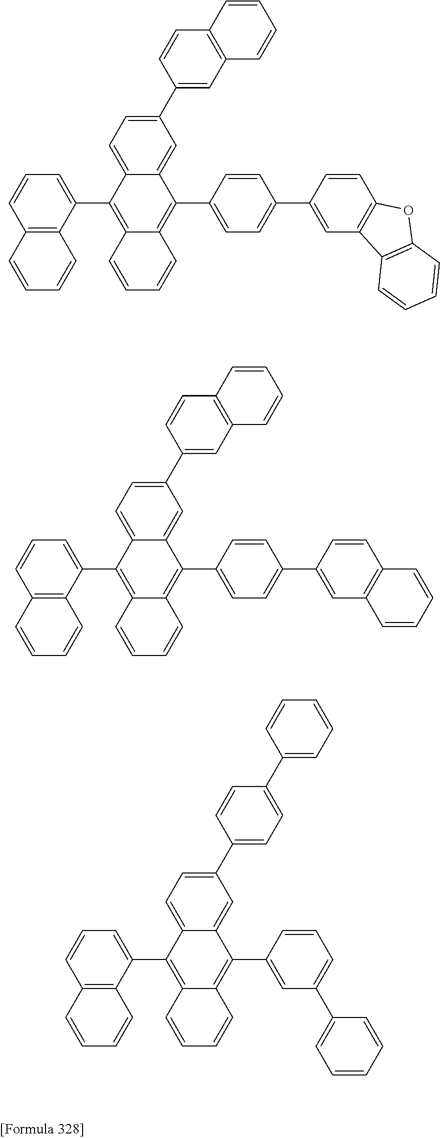

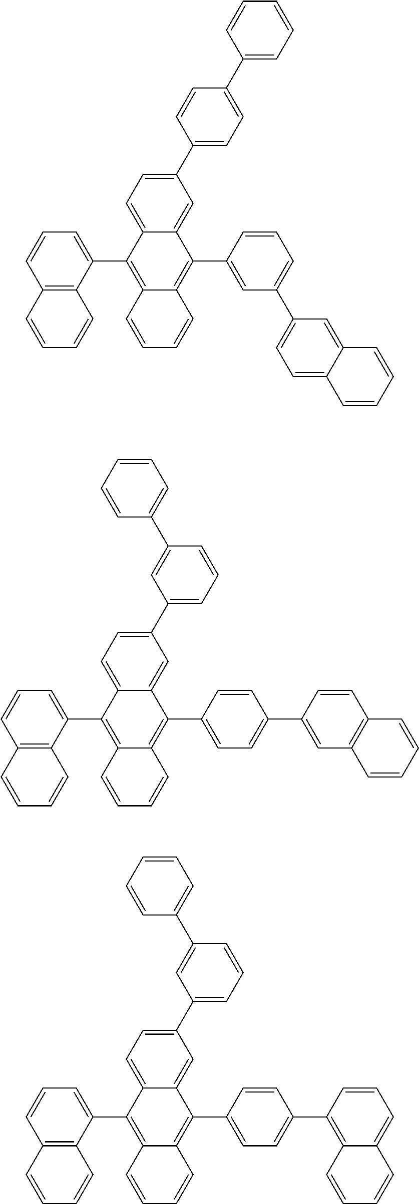

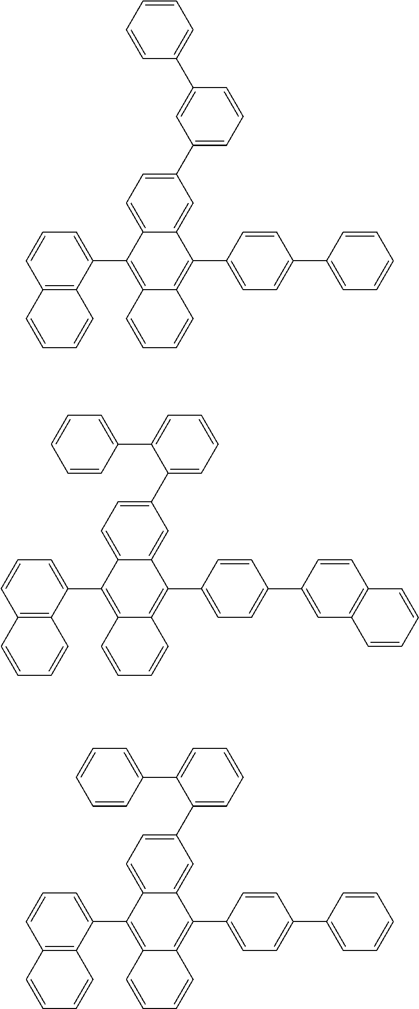

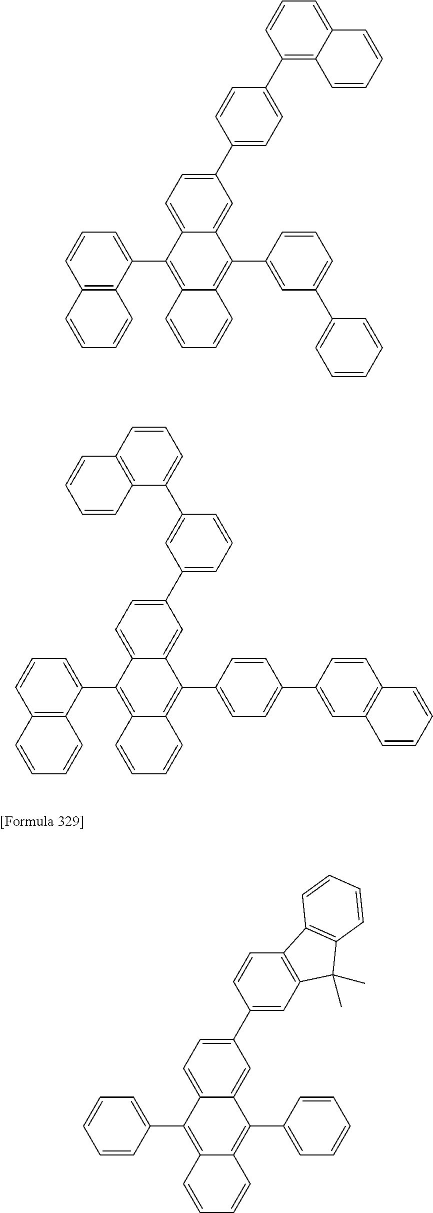

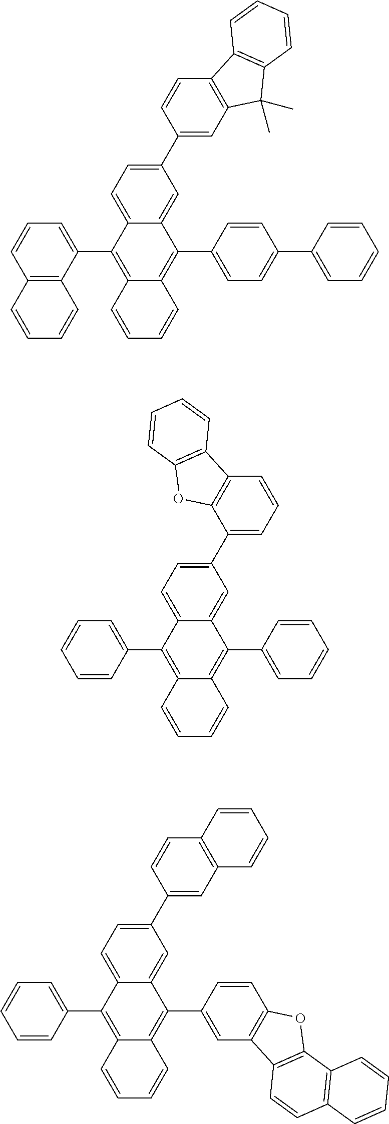

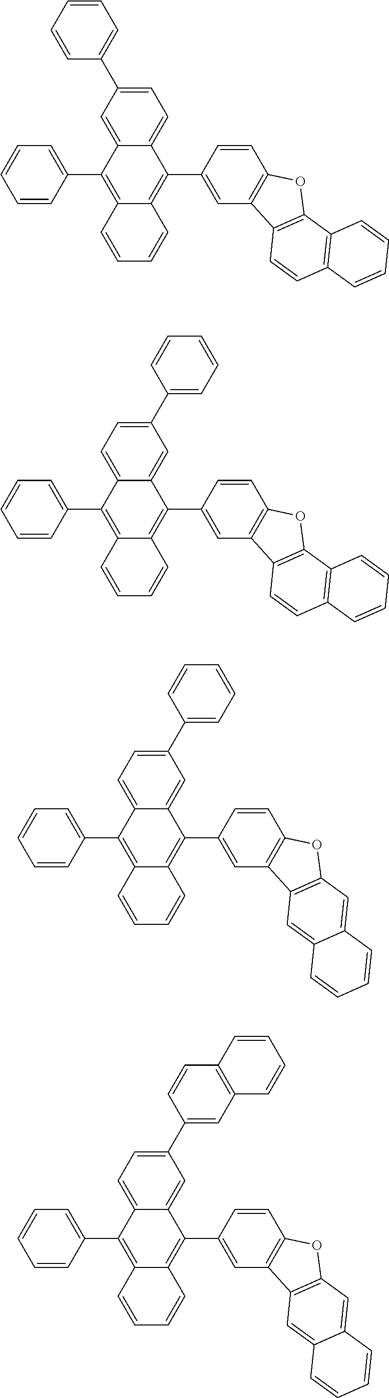 [SMILES: c1ccc(-c2ccc3c(-c4ccc5oc6c7ccccc7ccc6c5c4)c4ccccc4c(-c4ccccc4)c3c2)cc1 ; c1ccc(-c2ccc3c(-c4ccccc4)c4ccccc4c(-c4ccc5oc6c7ccccc7ccc6c5c4)c3c2)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1]

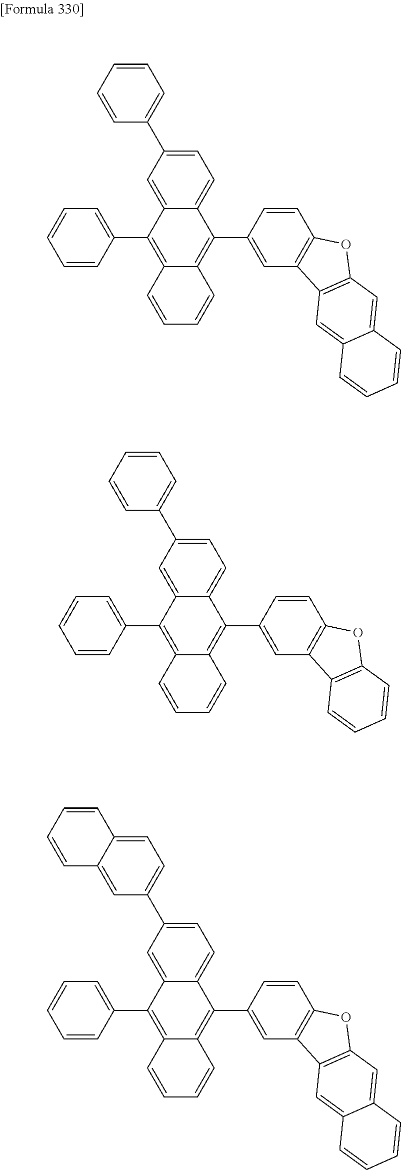 [SMILES: c1ccc(-c2ccc3c(-c4ccc5oc6cc7ccccc7cc6c5c4)c4ccccc4c(-c4ccccc4)c3c2)cc1 ; c1ccc(-c2ccc3c(-c4ccc5oc6ccccc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1]

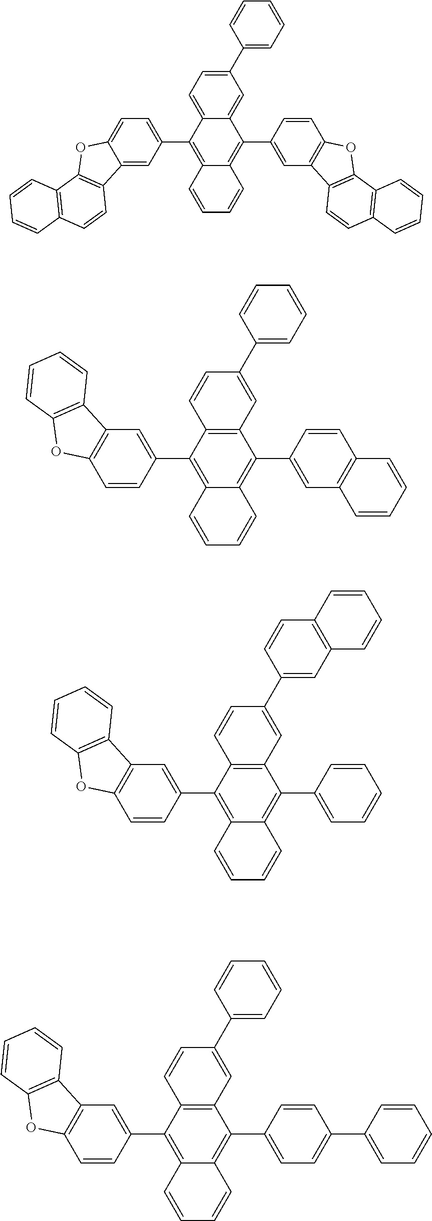 [SMILES: c1ccc(-c2ccc3c(-c4ccc5oc6c7ccccc7ccc6c5c4)c4ccccc4c(-c4ccc5oc6c7ccccc7ccc6c5c4)c3c2)cc1 ; c1ccc(-c2ccc3c(-c4ccc5oc6ccccc6c5c4)c4ccccc4c(-c4ccc5ccccc5c4)c3c2)cc1 ; c1ccc(-c2c3ccccc3c(-c3ccc4oc5ccccc5c4c3)c3ccc(-c4ccc5ccccc5c4)cc23)cc1 ; c1ccc(-c2ccc(-c3c4ccccc4c(-c4ccc5oc6ccccc6c5c4)c4ccc(-c5ccccc5)cc34)cc2)cc1]

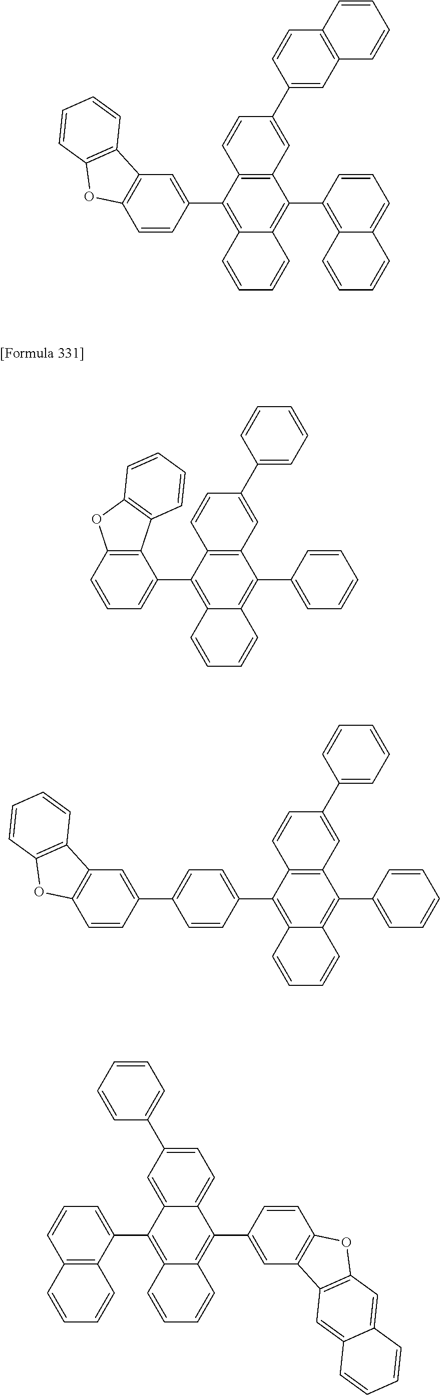 [SMILES: c1ccc2cc(-c3ccc4c(-c5ccc6oc7ccccc7c6c5)c5ccccc5c(-c5cccc6ccccc56)c4c3)ccc2c1 ; c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1]

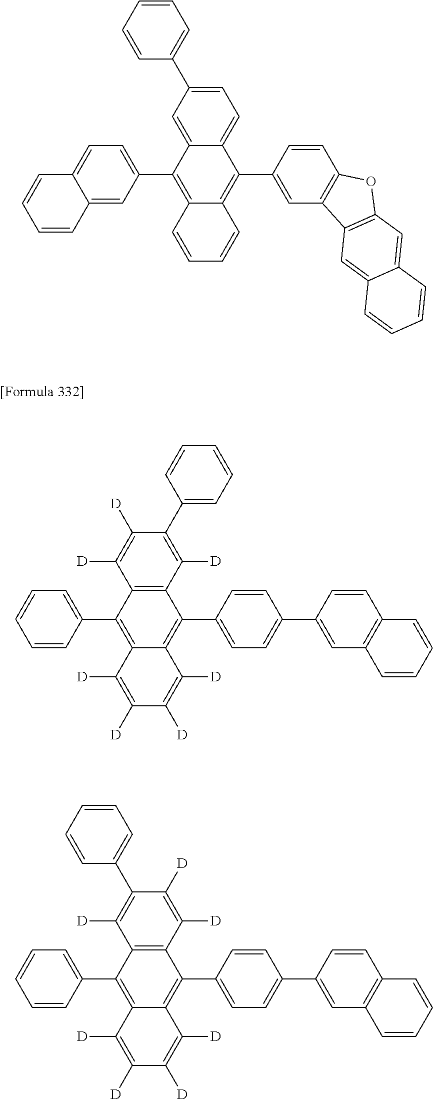 [SMILES: c1ccc(-c2ccc3c(-c4ccc5oc6cc7ccccc7cc6c5c4)c4ccccc4c(-c4ccc5ccccc5c4)c3c2)cc1 ; [2H]c1c([2H])c([2H])c2c(-c3ccc(-c4ccc5ccccc5c4)cc3)c3c([2H])c(-c4ccccc4)c([2H])c([2H])c3c(-c3ccccc3)c2c1[2H] ; [2H]c1c([2H])c([2H])c2c(-c3ccccc3)c3c([2H])c(-c4ccccc4)c([2H])c([2H])c3c(-c3ccc(-c4ccc5ccccc5c4)cc3)c2c1[2H]]

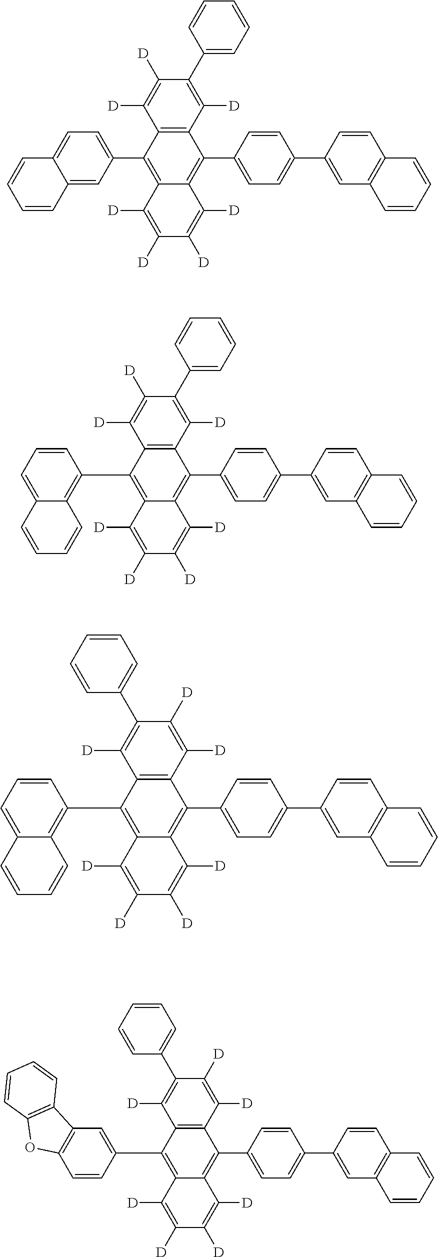 [SMILES: [2H]c1c([2H])c([2H])c2c(-c3ccc(-c4ccc5ccccc5c4)cc3)c3c([2H])c(-c4ccccc4)c([2H])c([2H])c3c(-c3ccc4ccccc4c3)c2c1[2H] ; [2H]c1c([2H])c([2H])c2c(-c3cccc4ccccc34)c3c([2H])c([2H])c(-c4ccccc4)c([2H])c3c(-c3ccc(-c4ccc5ccccc5c4)cc3)c2c1[2H] ; [2H]c1c([2H])c([2H])c2c(-c3cccc4ccccc34)c3c([2H])c(-c4ccccc4)c([2H])c([2H])c3c(-c3ccc(-c4ccc5ccccc5c4)cc3)c2c1[2H] ; [2H]c1c([2H])c([2H])c2c(-c3ccc4oc5ccccc5c4c3)c3c([2H])c(-c4ccccc4)c([2H])c([2H])c3c(-c3ccc(-c4ccc5ccccc5c4)cc3)c2c1[2H]]

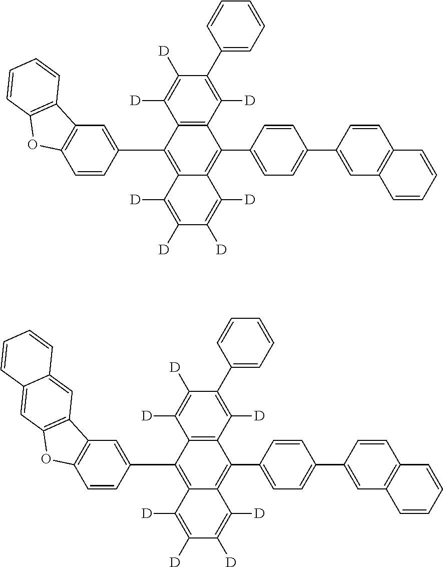 [SMILES: [2H]c1c([2H])c([2H])c2c(-c3ccc(-c4ccc5ccccc5c4)cc3)c3c([2H])c(-c4ccccc4)c([2H])c([2H])c3c(-c3ccc4oc5ccccc5c4c3)c2c1[2H] ; [2H]c1c([2H])c([2H])c2c(-c3ccc(-c4ccc5ccccc5c4)cc3)c3c([2H])c(-c4ccccc4)c([2H])c([2H])c3c(-c3ccc4oc5cc6ccccc6cc5c4c3)c2c1[2H]]

In an exemplary arrangement of the organic EL device of each exemplary embodiment, the first emitting region consists of the first emitting layer.

In an exemplary arrangement of the organic EL device of each exemplary embodiment, the first emitting region further includes the second emitting layer in addition to the first emitting layer.

In an exemplary arrangement of the organic EL device of each exemplary embodiment, the first emitting region consists of the first emitting layer and the second emitting layer.

In an exemplary arrangement of the organic EL device of each exemplary embodiment, the first emitting region includes at least the first emitting layer containing the first host material and the second emitting layer containing the second host material. The first host material and the second host material are mutually different.

In a case where the first emitting region at least includes the first emitting layer and the second emitting layer, luminous efficiency is improvable by using Triplet-Triplet-Annihilation (sometimes referred to as TTA).

TTA is a mechanism in which triplet excitons collide with one another to generate singlet excitons. The TTA mechanism is sometimes also referred to as a TTF mechanism as described in International Publication No. WO2010/134350.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of the conventional fluorescent device is believed to be 25%.

The behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as ³A*) collide with one another with an increase in density thereof, whereby a reaction shown by the following formula occurs. In the formula, ¹A represents the ground state and ¹A* represents the lowest singlet excitons.

$${}^{3} {A}^{*} + {}^{3} {A}^{*} \to ( 4 / 9 ) {}^{1} A + ( 1 / 9 ) {}^{1} {A}^{*} + ( 13 / 9 ) {}^{3} {A}^{*}$$

In other words, 5³A*→4¹A+1A′ is satisfied, and it is expected that, among triplet excitons initially generated, which account for 75%, one fifth thereof (i.e., 20%) is changed to singlet excitons. Accordingly, the amount of singlet excitons which contribute to emission is 40%, which is a value obtained by adding 15% (75%×(⅕)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(½)=37.5%) to 25% (the amount ratio of initially generated singlet excitons). At this time, the TTF ratio is 37.5/62.5=60%.

In an exemplary arrangement of the organic EL device of each exemplary embodiment, the triplet energy of the first host material T₁ (H1) and the triplet energy of the second host material T₁ (H2) preferably satisfy a relationship of a numerical formula (Numerical Formula 1) below, more preferably satisfy a relationship of a numerical formula (Numerical Formula 2) below.

$${T}_{1} ( H 2 ) > {T}_{1} ( H 1 ) \qquad ( Numerical Formula 1 )$$

$${T}_{1} ( H 2 ) - {T}_{1} ( H 1 ) > 0.03 eV \qquad ( Numerical Formula 2 )$$

In a case where an exemplary arrangement of the organic EL device of each exemplary embodiment includes the first emitting layer and the second emitting layer satisfying the numerical formula (Numerical Formula 1), the luminous efficiency of the device is improvable.

In an exemplary arrangement of the organic EL device of each exemplary embodiment, it is considered that since the relationship of the numerical formula (Numerical Formula 1) is satisfied, triplet excitons generated by recombination of holes and electrons in the second emitting layer and present on an interface between the second emitting layer and organic layer(s) in direct contact therewith are not likely to be quenched even under the presence of excessive carriers on the interface between the second emitting layer and the organic layer(s). For instance, the presence of a recombination region locally on an interface between the second emitting layer and a hole transporting layer or an electron blocking layer is considered to cause quenching by excessive electrons. Meanwhile, the presence of a recombination region locally on an interface between the second emitting layer and an electron transporting layer or a hole blocking layer is considered to cause quenching by excessive holes.

In an exemplary arrangement of the organic EL device of each exemplary embodiment, by including the first emitting layer and the second emitting layer in a manner to satisfy the relationship of the numerical formula (Numerical Formula 1), triplet excitons generated in the second emitting layer can transfer to the first emitting layer without being quenched by excessive carriers and be inhibited from back-transferring from the first emitting layer to the second emitting layer. Consequently, the first emitting layer exhibits the TTF mechanism to effectively generate singlet excitons, thereby improving the luminous efficiency.

Accordingly, the organic EL device includes, as different regions, the second emitting layer mainly generating triplet excitons and the first emitting layer mainly exhibiting the TTF mechanism using triplet excitons having transferred from the second emitting layer, and has a difference in triplet energy provided by using a compound having a smaller triplet energy than that of the second host material in the second emitting layer as the first host material in the first emitting layer. The luminous efficiency is thus improved.

In an exemplary arrangement of the organic EL device of each exemplary embodiment, the first emitting layer and the second emitting layer are in direct contact with each other.

Herein, a layer arrangement in which “the first emitting layer and the second emitting layer are in direct contact with each other” may include one of embodiments (LS1), (LS2), and (LS3) below.

(LS1) An embodiment in which a region containing both the first host material and the second host material is generated in a process of vapor-depositing the compound of the first emitting layer and vapor-depositing the compound of the second emitting layer, and is present on the interface between the first emitting layer and the second emitting layer.

(LS2) An embodiment in which in a case of containing an luminescent compound in the first emitting layer and the second emitting layer, a region containing the first host material, the second host material and the luminescent compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing an luminescent compound in the first emitting layer and the second emitting layer, a region containing the luminescent compound, a region containing the first host material or a region containing the second host material is generated in a process of vapor-depositing the compound of the first emitting layer and vapor-depositing the compound of the second emitting layer, and is present on the interface between the first emitting layer and the second emitting layer.

### Second Emitting Layer

In an exemplary arrangement of the organic EL device of each exemplary embodiment, the second emitting layer contains the second host material. The second host material, which is not particularly limited, may be selected from the group consisting of a first compound described below, a compound represented by the formula (H1), a compound represented by the formula (H10), and a compound represented by the formula (H20).

In an exemplary arrangement of the organic EL device of each exemplary embodiment, the second emitting layer contains a second luminescent compound. The second luminescent compound is not particularly limited.

In an exemplary arrangement of the organic EL device of each exemplary embodiment, the second emitting layer contains the second host material and the second luminescent compound.

In an exemplary arrangement of the organic EL device of each exemplary embodiment, the second luminescent compound is the same as or different from the first luminescent compound contained in the first emitting layer.

In an exemplary arrangement of the organic EL device of each exemplary embodiment, the second host material is a compound different from the first host material contained in the first emitting layer.

The second luminescent compound is preferably a compound that emits light having a maximum peak wavelength of 500 nm or less, more preferably a compound that emits light having a maximum peak wavelength in a range from 430 nm to 480 nm. The second luminescent compound is preferably a fluorescent compound that emits fluorescence having a maximum peak wavelength of 500 nm or less, more preferably a fluorescent compound that emits fluorescence having a maximum peak wavelength in a range from 430 nm to 480 nm.

In an exemplary arrangement of the organic EL device of each exemplary embodiment, the second emitting layer contains the second host material and the second luminescent compound that emits light having the maximum peak wavelength of 500 nm or less.

In an exemplary arrangement of the organic EL device of each exemplary embodiment, the second luminescent compound has a full width at half maximum in a range from 1 nm to 30 nm at a maximum peak.

A method of measuring the maximum peak wavelength of the compound is as follows.

In an exemplary arrangement of the organic EL device of each exemplary embodiment, the second luminescent compound is a compound containing no azine ring structure in a molecule.

In an exemplary arrangement of the organic EL device of each exemplary embodiment, the second luminescent compound is preferably not a boron-containing complex, more preferably not a complex.

As a compound that emits blue fluorescence and is usable for the second emitting layer, for instance, compounds such as a pyrene derivative, a styrylamine derivative, a chrysene derivative, a fluoranthene derivative, a fluorene derivative, a diamine derivative, and a triarylamine derivative are usable.

In an exemplary arrangement of the organic EL device of each exemplary embodiment, the second emitting layer does not contain a metal complex. Moreover, in an exemplary arrangement of the organic EL device of each exemplary embodiment, the second emitting layer does not contain a boron-containing complex.

In an exemplary arrangement of the organic EL device of each exemplary embodiment, the second emitting layer does not contain a phosphorescent material (dopant material).

In an exemplary arrangement of the organic EL device of each exemplary embodiment, the second emitting layer does not contain a heavy metal complex and a phosphorescent rare-earth metal complex.

In an exemplary arrangement of the organic EL device of each exemplary embodiment, the second emitting layer contains 0.5 mass % or more of the luminescent compound with respect to the total mass of the second emitting layer. The second emitting layer contains the luminescent compound preferably at 10 mass % or less, more preferably at 7 mass % or less, and still more preferably at 5 mass % less with respect to the total mass of the second emitting layer.

In an exemplary arrangement of the organic EL device of each exemplary embodiment, the second emitting layer contains the second host material preferably at 60 mass % or more, more preferably at 70 mass % or more, still more preferably at 80 mass % or more, still further more preferably at 90 mass % or more, and yet still further more preferably at 95 mass % or more, with respect to the total mass of the second emitting layer.

The second emitting layer preferably contains the second host material at 99.5 mass % or less with respect to the total mass of the second emitting layer.

When the second emitting layer contains the second host material and the second luminescent compound, the upper limit of the total of the content ratios of the second host material and the second luminescent compound is 100 mass %.

In the organic EL device of each exemplary embodiment, the second emitting layer may further contain any other material than the second host material and the second luminescent compound.

The second emitting layer may contain a single type of the second host material or may contain two or more types of the second host material. The second emitting layer may contain a single type of the second luminescent compound or may contain two or more types of the second luminescent compound.

In an exemplary arrangement of the organic EL device of each exemplary embodiment, a triplet energy of the first luminescent compound T₁(D1) and the triplet energy of the first host material T₁(H1) preferably satisfy a relationship of a numerical formula (Numerical Formula 4A) below.

$${T}_{1} ( D 1 ) > {T}_{1} ( H 1 ) \qquad ( Numerical Formula 4 A )$$

In an exemplary arrangement of the organic EL device of each exemplary embodiment, when the first luminescent compound and the first host material satisfy the relationship of the numerical formula (Numerical Formula 4A), in transfer of triplet excitons generated in the second emitting layer to the first emitting layer, the triplet excitons energy-transfer not onto the first luminescent compound having higher triplet energy but onto molecules of the first host material. In addition, triplet excitons generated by recombination of holes and electrons on the first host material do not transfer to the first luminescent compound having higher triplet energy. Triplet excitons generated by recombination on molecules of the first luminescent compound quickly energy-transfer to molecules of the first host material.

Triplet excitons in the first host material do not transfer to the first luminescent compound but efficiently collide with one another on the first host material to generate singlet excitons by the TTF phenomenon.

In an exemplary arrangement of the organic EL device of each exemplary embodiment, a singlet energy of the first host material S₁ (H1) and a singlet energy of the first luminescent compound S₁ (D1) preferably satisfy a relationship of a numerical formula (Numerical Formula 4) below.

$${S}_{1} ( H 1 ) > {S}_{1} ( D 1 ) \qquad ( Numerical Formula 4 )$$

In an exemplary arrangement of the organic EL device of each exemplary embodiment, when the first luminescent compound and the first host material satisfy the relationship of the numerical formula (Numerical formula 4), due to the singlet energy of the first luminescent compound being smaller than the singlet energy of the first host material, singlet excitons generated by the TTF phenomenon energy-transfer from the first host material to the first luminescent compound, thereby contributing to emission (preferably fluorescence) of the first luminescent compound.

In an exemplary arrangement of the organic EL device of each exemplary embodiment, when the first emitting layer and the second emitting layer are layered in this order from a side on which the anode is provided, the electron mobility of the second host material μe(H2) and the electron mobility of the first host material μe(H1) preferably satisfy a formula (Numerical Formula 3) below. When the first host material and the second host material satisfy a relationship of the numerical formula (Numerical Formula 3), a recombination ability between holes and electrons in the second emitting layer is improved.

$$μ e ( H 1 ) > μ e ( H 2 ) \qquad ( Numerical Formula 3 )$$

In an exemplary arrangement of the organic EL device of each exemplary embodiment, when the second emitting layer and the first emitting layer are layered in this order from a side on which the anode is provided, the hole mobility of the second host material μh(H2) and the hole mobility of the first host material μh(H1) preferably satisfy a formula (Numerical Formula 31) below.

$$μ h ( H 2 ) > μ h ( H 1 ) \qquad ( Numerical Formula 31 )$$

In an exemplary arrangement of the organic EL device of each exemplary embodiment, when the second emitting layer and the first emitting layer are layered in this order from a side on which the anode is provided, the hole mobility of the second host material μh(H2), the electron mobility of the second host material μe(H2), the hole mobility of the first host material μh(H1), and the electron mobility of the first host material μe(H1) preferably satisfy a formula (Numerical Formula 32) below.

$$( μ e ( H 1 ) / μ h ( H 1 ) ) > ( μ e ( H 2 ) / μ h ( H 2 ) ) \qquad ( Numerical Formula 32 )$$

In an exemplary arrangement of the organic EL device of each exemplary embodiment, a singlet energy of the second host material S₁ (H2) and a singlet energy of the second luminescent compound S₁ (D2) preferably satisfy a relationship of a numerical formula (Numerical Formula 20) below.

$${S}_{1} ( H 2 ) > {S}_{1} ( D 2 ) \qquad ( Numerical Formula 20 )$$

When the second host material and the second luminescent compound satisfy the relationship of the numerical formula (Numerical Formula 20), singlet excitons generated on the second host material easily energy-transfer from the second host material to the second luminescent compound, thereby contributing to emission (preferably fluorescence) of the second luminescent compound.

In an exemplary arrangement of the organic EL device of each exemplary embodiment, the triplet energy of the second host material T₁ (H2) and the triplet energy of the second luminescent compound T₁ (D2) preferably satisfy a relationship of a numerical formula (Numerical Formula 20A) below.

$${T}_{1} ( D 2 ) > {T}_{1} ( H 2 ) \qquad ( Numerical Formula 20 A )$$

When the second host material and the second luminescent compound satisfy the relationship of the numerical formula (Numerical Formula 20A), triplet excitons generated in the second emitting layer are transferred not onto the second luminescent compound having higher triplet energy but onto the second host material, thereby being easily transferred to the first emitting layer.

Methods of measuring the triplet energy T₁, the singlet energy S₁, the hole mobility and the electron mobility will be described below.

In an exemplary arrangement of the organic EL device of each exemplary embodiment, a film thickness of the first emitting layer is preferably 5 nm or more, more preferably 15 nm or more. In a case where the first emitting region includes the second emitting layer, when the film thickness of the first emitting layer is 5 nm or more, it is easy to inhibit triplet excitons having transferred from the second emitting layer to the first emitting layer from returning to the second emitting layer. Further, when the film thickness of the first emitting layer is 5 nm or more, triplet excitons can be sufficiently separated from the recombination portion in the second emitting layer.

In an exemplary arrangement of the organic EL device of each exemplary embodiment, the film thickness of the first emitting layer is preferably 20 nm or less. With the film thickness of the first emitting layer of 20 nm or less, a density of triplet excitons in the first emitting layer is improvable to further facilitate occurrence of the TTF phenomenon.

In an exemplary arrangement of the organic EL device of each exemplary embodiment, the film thickness of the first emitting layer is preferably in a range from 5 nm to 20 nm.

In an exemplary arrangement of the organic EL device of each exemplary embodiment, the film thickness of the second emitting layer is preferably 3 nm or more, more preferably 5 nm or more. When the film thickness of the second emitting layer is 3 nm or more, the film thickness is sufficiently large to cause recombination of holes and electrons in the second emitting layer.

In an exemplary arrangement of the organic EL device of each exemplary embodiment, the film thickness of the second emitting layer is preferably 15 nm or less, more preferably 10 nm or less. When the film thickness of the second emitting layer is 15 nm or less, the film thickness is sufficiently thin to allow for transfer of triplet excitons to the first emitting layer.

In an exemplary arrangement of the organic EL device of each exemplary embodiment, the film thickness of the second emitting layer is more preferably in a range from 3 nm to 15 nm.

### First Host Material and Second Host Material

In an exemplary arrangement of the organic EL device of each exemplary embodiment, the first host material and the second host material are also each preferably the first compound. The first compound is, for instance, a compound selected from the group consisting of compounds represented by a formula (1), a formula (1X), a formula (12X), a formula (13X), a formula (14X), a formula (15X), a formula (16X), a formula (1000B), a formula (16X), a formula (17X-1), a formula (17X-2), a formula (17X-3), and a formula (18) below.

Moreover, the first compound is also usable as the first host material and the second host material.

### Compound Represented by Formula (1)

In an exemplary arrangement of the organic EL device of each exemplary embodiment, the first compound is a compound represented by the formula (1) below. The first compound represented by the formula (1) has at least one group represented by a formula (11) below.

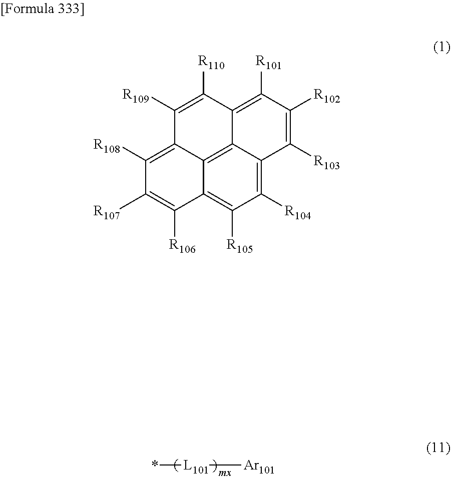

In the formula (1):
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 0, 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring represented by the formula (1).

In the first compound represented by the formula (1), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary embodiment, Ar₁₀₁ is preferably a substituted or unsubstituted aryl group having 6 to 50 carbon atoms.

In an exemplary embodiment, Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted pyrenyl group, a substituted or unsubstituted phenanthryl group, or a substituted or unsubstituted fluorenyl group.

In an exemplary embodiment, the first compound is preferably represented by a formula (101) below.

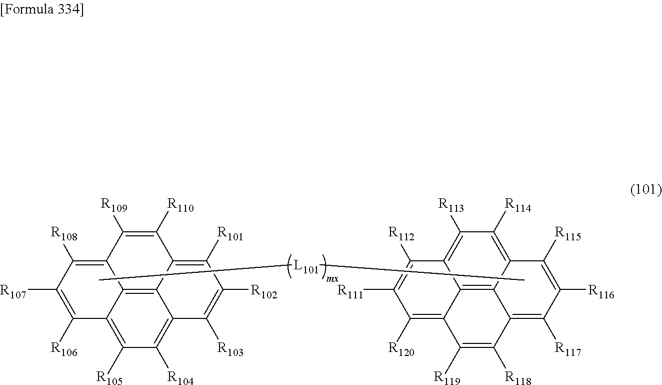

In the formula (101):
R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 50 ring carbon atoms, or a substituted or unsubstituted divalent heterocyclic group having 5 to 50 ring atoms;
mx is 0, 1, 2, 3, 4, or 5; and
when two or more L₁₀₁ are present, the two or more L₁₀₁ are mutually the same or different.

In an exemplary embodiment, L₁₀₁ is preferably a single bond or a substituted or unsubstituted arylene group having 6 to 50 ring carbon atoms.

In an exemplary embodiment, two or more of R₁₀₁ to R₁₁₀ are each preferably a group represented by the formula (11).

In an exemplary embodiment, it is preferable that two or more of R₁₀₁ to R₁₁₀ are each a group represented by the formula (11) and Ar₁₀₁ is a substituted or unsubstituted aryl group having 6 to 50 ring carbon atoms.

In an exemplary embodiment, it is preferable that Ar₁₀₁ is not a substituted or unsubstituted pyrenyl group, L₁₀₁ is not a substituted or unsubstituted pyrenylene group, and a substituted or unsubstituted aryl group having 6 to 50 ring carbon atoms as R₁₀₁ to R₁₁₀ not being a group represented by the formula (11) is not a substituted or unsubstituted pyrenyl group.

In an exemplary embodiment, R₁₀₁ to R₁₁₀ not being a group represented by the formula (11) are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary embodiment, R₁₀₁ to R₁₁₀ not being a group represented by the formula (11) are preferably each independently a hydrogen atom, a substituted or unsubstituted alkyl group having 1 to 50 carbon atoms, or a substituted or unsubstituted cycloalkyl group having 3 to 50 ring carbon atoms.

In an exemplary embodiment, R₁₀₁ to R₁₁₀ not being the group represented by the formula (11) are each preferably a hydrogen atom.

### Compound Represented by Formula (1X)

In an exemplary arrangement of the organic EL device of each exemplary embodiment, the first compound is a compound represented by a formula (1X) below.

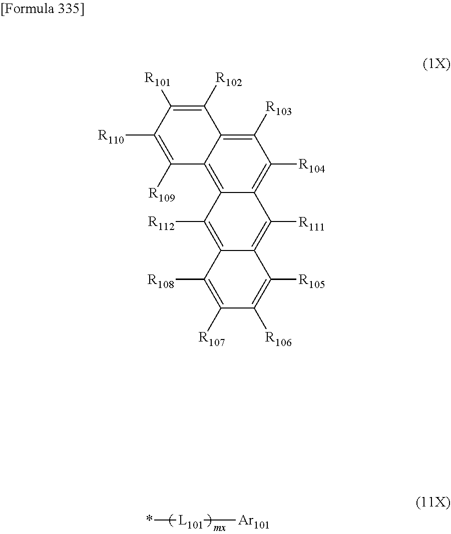

In the formula (1X):
R₁₀₁ to R₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11X);
at least one of R₁₀₁ to R₁₁₂ is a group represented by the formula (11X);
when a plurality of groups represented by the formula (11X) are present, the plurality of groups represented by the formula (11X) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different; and
* in the formula (11X) represents a bonding position to a benz[a]anthracene ring in the formula (1X).

In an exemplary arrangement of the organic EL device of each exemplary embodiment, the group represented by the formula (11X) is a group represented by a formula (111X) below.

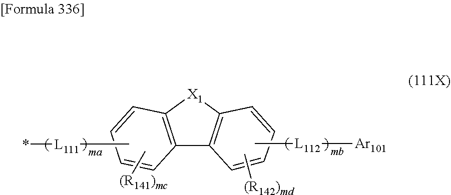

In the formula (111X):
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3 or 4;
mb is 1, 2, 3 or 4;
ma+mb is 2, 3, or 4;
Ar₁₀₁ represents the same as Ar₁₀₁ in the formula (11X);
R₁₄₁, R₁₄₂, R₁₄₃, R₁₄₄ and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

Among positions *1 to *8 of carbon atoms in a cyclic structure represented by a formula (111aX) below in the group represented by the formula (111X), L₁₁₁ is bonded to one of the positions *1 to *4, R₁₄₁ is bonded to each of three positions of the rest of *1 to *4, L₁₁₂ is bonded to one of the positions *5 to *8, and R₁₄₂ is bonded to each of three positions of the rest of *5 to *8.

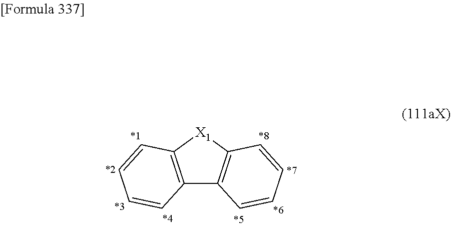

For instance, the group represented by the formula (111X), in which L₁₁₁ is bonded to a carbon atom at *2 in the cyclic structure represented by the formula (111aX) and L₁₁₂ is bonded to a carbon atom at *7 in the cyclic structure represented by the formula (111aX), is represented by a formula (111bX) below.

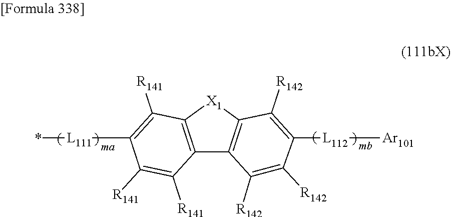

In the formula (111bX):
X₁, L₁₁₁, L₁₁₂, ma, mb, Ar₁₀₁, R₁₄₁, R₁₄₂, R₁₄₃, R₁₄₄ and R₁₄₅ each independently represent the same as X₁, L₁₁₁, L₁₁₂, ma, mb, Ar₁₀₁, R₁₄₁, R₁₄₂, R₁₄₃, R₁₄₄ and R₁₄₅ in the formula (111X);
a plurality of R₁₄₁ are mutually the same or different; and
a plurality of R₁₄₂ are mutually the same or different.

In the organic EL device of each exemplary embodiment, the group represented by the formula (111X) is preferably a group represented by the formula (111bX).

In the compound represented by the formula (1X), preferably, ma is 1 or 2 and mb is 1 or 2.

In the compound represented by the formula (1X), preferably, ma is 1 and mb is 1.

In the compound represented by the formula (1X), Ar₁₀₁ is preferably a substituted or unsubstituted aryl group having 6 to 50 ring carbon atoms.

In the compound represented by the formula (1X), Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted benz[a]anthryl group, a substituted or unsubstituted pyrenyl group, a substituted or unsubstituted phenanthryl group, or a substituted or unsubstituted fluorenyl group.

The compound represented by the formula (1X) is also preferably represented by a formula (101X) below.

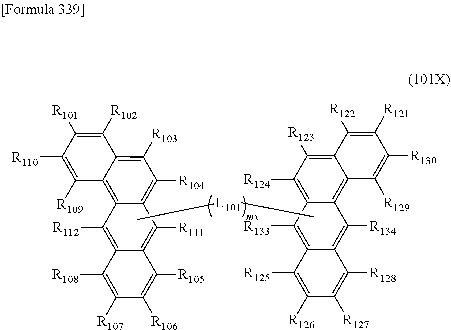

In the formula (101X):
one of R₁₁₁ and R₁₁₂ represents a bonding position to L₁₀₁ and one of R₁₃₃ and R₁₃₄ represents a bonding position to L₁₀₁;
R₁₀₁ to R₁₁₀, R₁₂₁ to R₁₃₀, R₁₁₁ or R₁₁₂ not being the bonding position to L₁₀₁, and R₁₃₃ or R₁₃₄ not being the bonding position to L₁₀₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀₁ is a single bond, a substituted or unsubstituted arylene group having 6 to 50 ring carbon atoms, or a substituted or unsubstituted divalent heterocyclic group having 5 to 50 ring atoms;
mx is 1, 2, 3, 4, or 5; and
when two or more L₁₀₁ are present, the two or more L₁₀₁ are mutually the same or different.

In the compound represented by the formula (1X), L₁₀₁ is preferably a single bond or a substituted or unsubstituted arylene group having 6 to 50 ring carbon atoms.

The compound represented by the formula (1X) is also preferably represented by a formula (102X) below.

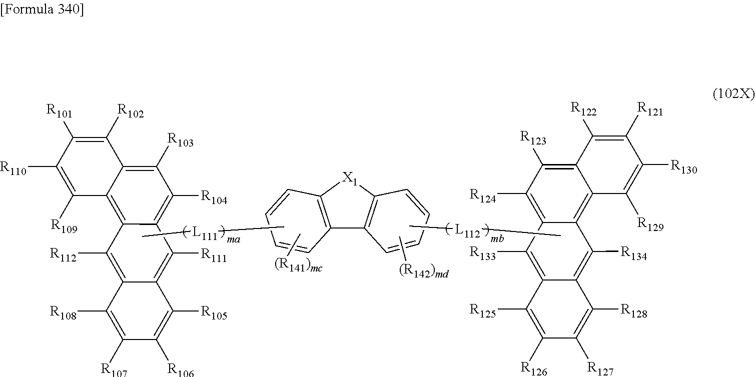

In the formula (102X):
one of R₁₁₁ and R₁₁₂ represents a bonding position to L₁₁₁ and one of R₁₃₃ and R₁₃₄ represents a bonding position to L₁₁₂;
R₁₀₁ to R₁₁₀, R₁₂₁ to R₁₃₀, R₁₁₁ or R₁₁₂ not being the bonding position to L₁₁₁, and R₁₃₃ or R₁₃₄ not being the bonding position to L₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
X₁ is CR₁₄₃R₁₄₄, an oxygen atom, a sulfur atom, or NR₁₄₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3 or 4;
mb is 1, 2, 3 or 4;
ma+mb is 2, 3, 4, or 5;
R₁₄₁, R₁₄₂, R₁₄₃, R₁₄₄ and R₁₄₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₄₁ are mutually the same or different;
md is 3; and
three R₁₄₂ are mutually the same or different.

In the compound represented by the formula (1X), preferably, ma is 1 or 2 and mb is 1 or 2 in the formula (102X).

In the compound represented by the formula (1X), preferably, ma is 1 and mb is 1 in the formula (102X).

In the compound represented by the formula (1X), the group represented by the formula (11X) is also preferably a group represented by a formula (11AX) or a group represented by a formula (11BX) below.

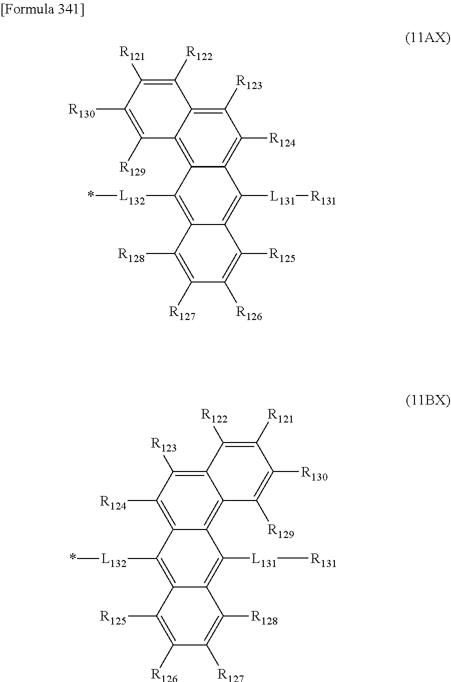

In the formulae (11AX) and (11BX):
R₁₂₁ to R₁₃₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
when a plurality of groups represented by the formula (11AX) are present, the plurality of groups represented by the formula (11AX) are mutually the same or different;
when a plurality of groups represented by the formula (11BX) are present, the plurality of groups represented by the formula (11BX) are mutually the same or different;
L₁₃₁ and L₁₃₂ are each independently a single bond, a substituted or unsubstituted arylene group having 6 to 50 ring carbon atoms, or a substituted or unsubstituted divalent heterocyclic group having 5 to 50 ring atoms; and
* in each of the formulae (11AX) and (11BX) represents a bonding position to a benz[a]anthracene ring in the formula (1X).

The compound represented by the formula (1X) is also preferably represented by a formula (103X) below.

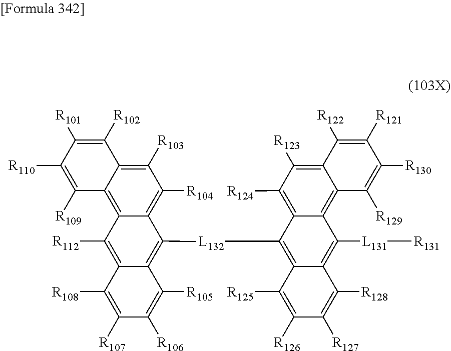

In the formula (103X):
R₁₀₁ to R₁₁₀ and R₁₁₂ respectively represent the same as R₁₀₁ to R₁₁₀ and R₁₁₂ in the formula (1X); and
R₁₂₁ to R₁₃₁, L₁₃₁, and L₁₃₂ respectively represent the same as R₁₂₁ to R₁₃₁, L₁₃₁, and L₁₃₂ in the formula (11BX).

In the compound represented by the formula (1X), L₁₃₁ is also preferably a substituted or unsubstituted arylene group having 6 to 50 ring carbon atoms.

In the compound represented by the formula (1X), L₁₃₂ is also preferably a substituted or unsubstituted arylene group having 6 to 50 ring carbon atoms.

In the compound represented by the formula (1X), also preferably, two or more of R₁₀₁ to R₁₁₂ are each a group represented by the formula (11).

In the compound represented by the formula (1X), it is preferable that two or more of R₁₀₁ to R₁₁₂ are each a group represented by the formula (11X) and Ar₁₀₁ in the formula (11X) is a substituted or unsubstituted aryl group having 6 to 50 ring carbon atoms.

In the compound represented by the formula (1X), it is also preferable that Ar₁₀₁ is not a substituted or unsubstituted benz[a]anthryl group, L₁₀₁ is not a substituted or unsubstituted benz[a]anthrylene group, and a substituted or unsubstituted aryl group having 6 to 50 ring carbon atoms as R₁₀₁ to R₁₁₀ not being a group represented by the formula (11X) is not a substituted or unsubstituted benz[a]anthryl group.

In the compound represented by the formula (1X), R₁₀₁ to R₁₁₂ not being a group represented by the formula (11X) are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (1X), R₁₀₁ to R₁₁₂ not being a group represented by the formula (11X) are preferably each a hydrogen atom, a substituted or unsubstituted alkyl group having 1 to 50 carbon atoms, or a substituted or unsubstituted cycloalkyl group having 3 to 50 ring carbon atoms.

In the compound represented by the formula (1X), R₁₀₁ to R₁₁₂ not being the group represented by the formula (11X) are each preferably a hydrogen atom.

### Compound Represented by Formula (12X)

In an exemplary arrangement of the organic EL device of each exemplary embodiment, the first compound is a compound represented by a formula (12X) below.

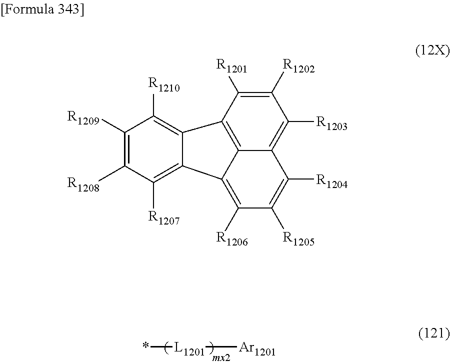

In the formula (12X):
at least one combination of adjacent two or more of R₁₂₀₁ to R₁₂₁₀ are mutually bonded to form a substituted or unsubstituted monocyclic ring, or mutually bonded to form a substituted or unsubstituted fused ring;
R₁₂₀₁ to R₁₂₁₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (121);
at least one of a substituent, if present, for the substituted or unsubstituted monocyclic ring, a substituent, if present, for the substituted or unsubstituted fused ring, or R₁₂₀₁ to R₁₂₁₀ is a group represented by the formula (121);
when a plurality of groups represented by the formula (121) are present, the plurality of groups represented by the formula (121) are mutually the same or different;
L₁₂₀₁ is a single bond, a substituted or unsubstituted arylene group having 6 to 50 ring carbon atoms, or a substituted or unsubstituted divalent heterocyclic group having 5 to 50 ring atoms;
Ar₁₂₀₁ is a substituted or unsubstituted aryl group having 6 to 50 ring carbon atoms, or a substituted or unsubstituted heterocyclic group having 5 to 50 ring atoms;
mx2 is 0, 1, 2, 3, 4, or 5;
when two or more L₁₂₀₁ are present, the two or more L₁₂₀₁ are mutually the same or different;
when two or more Ar₁₂₀₁ are present, the two or more Ar₁₂₀₁ are mutually the same or different; and
* in the formula (121) represents a bonding position to a ring represented by the formula (12X).

In the formula (12X), combinations of adjacent two of R₁₂₀₁ to R₁₂₁₀ refer to a combination of R₁₂₀₁ and R₁₂₀₂, a combination of R₁₂₀₂ and R₁₂₀₃, a combination of R₁₂₀₃ and R₁₂₀₄, a combination of R₁₂₀₄ and R₁₂₀₅, a combination of R₁₂₀₅ and R₁₂₀₆, a combination of R₁₂₀₇ and R₁₂₀₈, a combination of R₁₂₀₈ and R₁₂₀₉, and a combination of R₁₂₀₉ and R₁₂₁₀.

### Compound Represented by Formula (13X)

In an exemplary arrangement of the organic EL device of each exemplary embodiment, the first compound is a compound represented by a formula (13X) below.

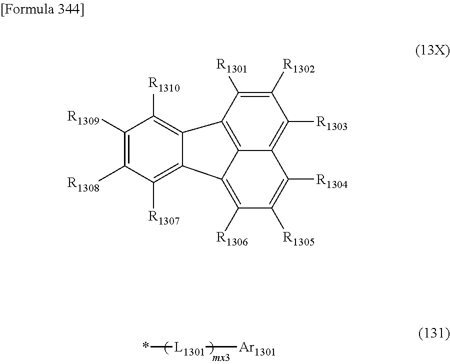

In the formula (13X):
R₁₃₀₁ to R₁₃₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (131);
at least one of R₁₃₀₁ to R₁₃₁₀ is a group represented by the formula (131);
when a plurality of groups represented by the formula (131) are present, the plurality of groups represented by the formula (131) are mutually the same or different;
L₁₃₀₁ is a single bond, a substituted or unsubstituted arylene group having 6 to 50 ring carbon atoms, or a substituted or unsubstituted divalent heterocyclic group having 5 to 50 ring atoms;
Ar₁₃₀₁ is a substituted or unsubstituted aryl group having 6 to 50 ring carbon atoms, or a substituted or unsubstituted heterocyclic group having 5 to 50 ring atoms;
mx3 is 0, 1, 2, 3, 4, or 5;
when two or more L₁₃₀₁ are present, the two or more L₁₃₀₁ are mutually the same or different;
when two or more Ar₁₃₀₁ are present, the two or more Ar₁₃₀₁ are mutually the same or different; and
* in the formula (131) represents a bonding position to a fluoranthene ring represented by the formula (13X).

In the organic EL device of each exemplary embodiment, none of combinations of adjacent two or more of R₁₃₀₁ to R₁₃₁₀ not being the group represented by the formula (131) are bonded to each other. Combinations of adjacent two of R₁₃₀₁ to R₁₃₁₀ in the formula (13X) refer to a combination of R₁₃₀₁ and R₁₃₀₂, a combination of R₁₃₀₂ and R₁₃₀₃, a combination of R₁₃₀₃ and R₁₃₀₄, a combination of R₁₃₀₄ and R₁₃₀₅, a combination of R₁₃₀₅ and R₁₃₀₆, a combination of R₁₃₀₇ and R₁₃₀₈, a combination of R₁₃₀₈ and R₁₃₀₉, and a combination of R₁₃₀₉ and R₁₃₁₀.

Compound Represented by Formula (14X) In an exemplary arrangement of the organic EL device of each exemplary embodiment, the first compound is a compound represented by a formula (14X) below.

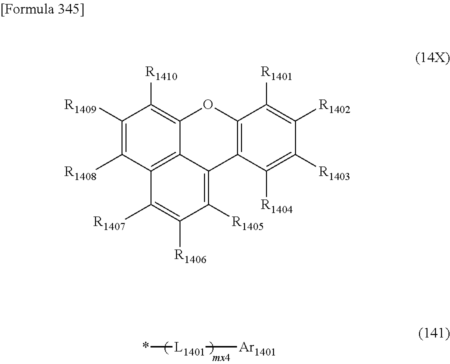

In the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4, or 5;
when two or more L₁₄₀₁ are present, the two or more L₁₄₀₁ are mutually the same or different;
when two or more Ar₁₄₀₁ are present, the two or more Ar₁₄₀₁ are mutually the same or different; and
* in the formula (141) represents a bonding position to a ring represented by the formula (14X).

### Compound Represented by Formula (15X)

In an exemplary arrangement of the organic EL device of each exemplary embodiment, the first compound is a compound represented by a formula (15X) below.

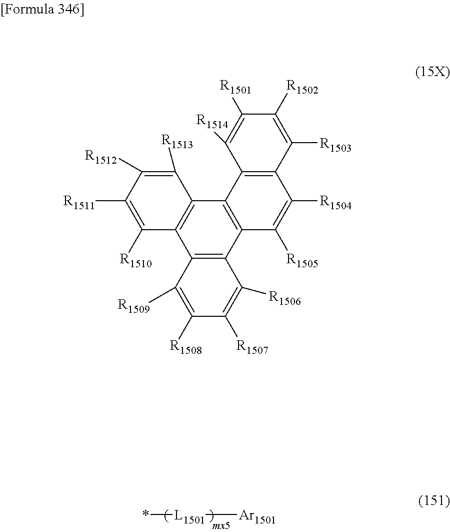

In the formula (15X):
R₁₅₀₁ to R₁₅₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (151);
at least one of R₁₅₀₁ to R₁₅₁₄ is a group represented by the formula (151);
when a plurality of groups represented by the formula (151) are present, the plurality of groups represented by the formula (151) are mutually the same or different;
L₁₅₀₁ is a single bond, a substituted or unsubstituted arylene group having 6 to 50 ring carbon atoms, or a substituted or unsubstituted divalent heterocyclic group having 5 to 50 ring atoms;
Ar₁₅₀₁ is a substituted or unsubstituted aryl group having 6 to 50 ring carbon atoms, or a substituted or unsubstituted heterocyclic group having 5 to 50 ring atoms;
mx5 is 0, 1, 2, 3, 4, or 5;
when two or more L₁₅₀₁ are present, the two or more L₁₅₀₁ are mutually the same or different;
when two or more Ar₁₅₀₁ are present, the two or more Ar₁₅₀₁ are mutually the same or different; and
* in the formula (151) represents a bonding position to a ring represented by the formula (15X).

Compound Represented by Formula (16X) In an exemplary arrangement of the organic EL device of each exemplary embodiment, the first compound is a compound represented by a formula (16X) below.

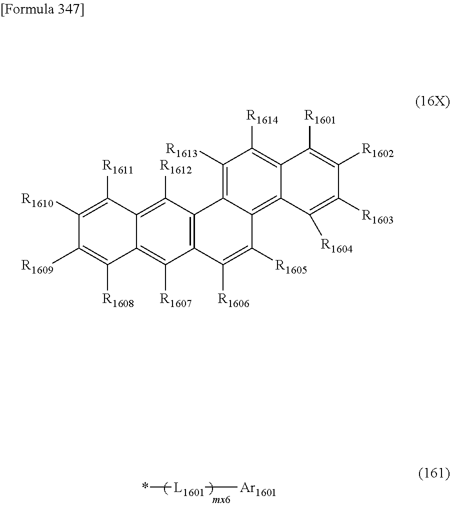

In the formula (16X):
R₁₆₀₁ to R₁₆₁₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (161);
at least one of R₁₆₀₁ to R₁₆₁₄ is a group represented by the formula (161);
when a plurality of groups represented by the formula (161) are present, the plurality of groups represented by the formula (161) are mutually the same or different;
L₁₆₀₁ is a single bond, a substituted or unsubstituted arylene group having 6 to 50 ring carbon atoms, or a substituted or unsubstituted divalent heterocyclic group having 5 to 50 ring atoms;
Ar₁₆₀₁ is a substituted or unsubstituted aryl group having 6 to 50 ring carbon atoms, or a substituted or unsubstituted heterocyclic group having 5 to 50 ring atoms;
mx6 is 0, 1, 2, 3, 4, or 5;
when two or more L₁₆₀₁ are present, the two or more L₁₆₀₁ are mutually the same or different;
when two or more Ar₁₆₀₁ are present, the two or more Ar₁₆₀₁ are mutually the same or different; and
* in the formula (161) represents a bonding position to a ring represented by the formula (16X).

Compound Represented by Formula (1000B) In an exemplary arrangement of the organic EL device of each exemplary embodiment, the first compound is a compound represented by the formula (1000B) below.

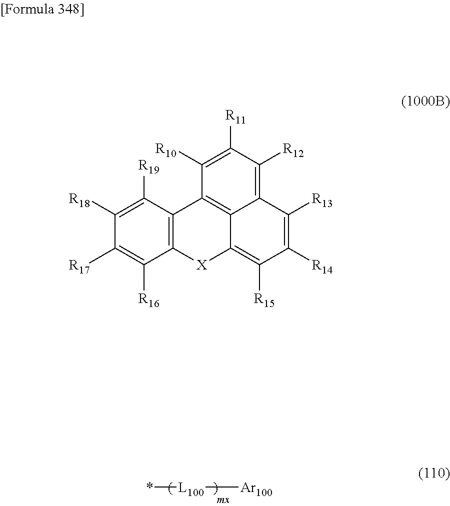

In the formula (1000B):
X is an oxygen atom or a sulfur atom;
at least one combination of adjacent two or more of R₁₀ to R₁₉ are mutually bonded to form a substituted or unsubstituted monocyclic ring, mutually bonded to form a substituted or unsubstituted fused ring, or not mutually bonded;
R₁₀ to R₁₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (110);
at least one of R₁₀ to R₁₉ is a group represented by the formula (110);
when a plurality of groups represented by the formula (110) are present, the plurality of groups represented by the formula (110) are mutually the same or different;
L₁₀₀ is a single bond, a substituted or unsubstituted arylene group having 6 to 50 ring carbon atoms, or a substituted or unsubstituted divalent heterocyclic group having 5 to 50 ring atoms;
mx is 1, 2, or 3;
when two or more L₁₀₀ are present, the two or more L₁₀₀ are mutually the same or different;
Ar₁₀₀ is a substituted or unsubstituted aryl group having three or more rings, or a substituted or unsubstituted heterocyclic group having two or more aromatic rings and one or more heterocycles;
Ar₁₀₀ contains no anthracene ring;
when two or more Ar₁₀₀ are present, the two or more Ar₁₀₀ are mutually the same or different;
in the formula (110) represents a bonding position;
R₉₀₁, R₉₀₂, R₉₀₃, R₉₀₄, R₉₀₅, R₉₀₆, R₉₀₇, R₈₀₁ and R₈₀₂ in the first compound represented by the formula (1000B) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the formula (1000B), X is preferably an oxygen atom.

The compound represented by the formula (1000B) is preferably a compound that has at least one group represented by the formula (110) and is represented by a formula (100) below.

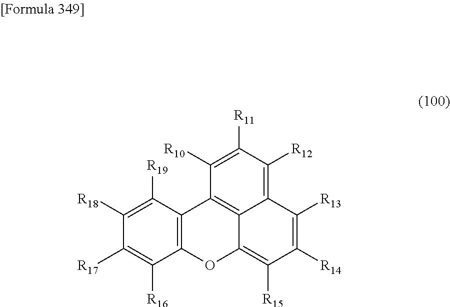

In the formula (100): R₁₀ to R₁₉ each independently represent the same as R₁₀ to R₁₉ in the formula (1000B); and Ar₁₀₀, L₁₀₀ and mx respectively represent the same as Ar₁₀₀, L₁₀₀ and mx in the formula (110).

The compound represented by the formula (1000B) is also preferably a compound represented by a formula (101) or (102) below.

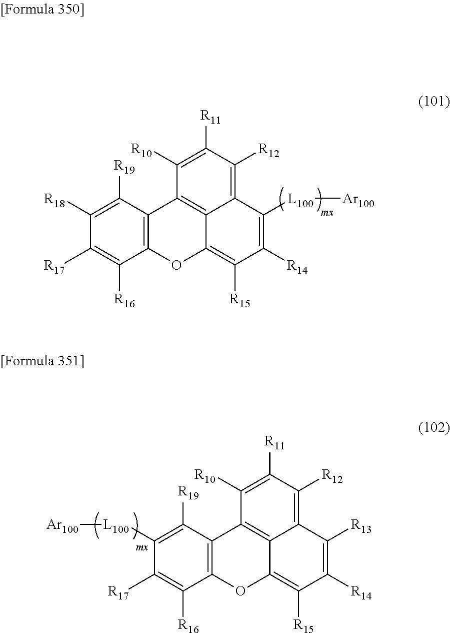

In the formulae (101) and (102): R₁₀ to R₁₉ each independently represent the same as R₁₀ to R₁₉ in the formula (1000B); and Ar₁₀₀, L₁₀₀ and mx respectively represent the same as Ar₁₀₀, L₁₀₀ and mx in the formula (110).

In the formula (1000B), R₁₀ to R₁₉ not being the group represented by the formula (110) are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the formula (1000B), R₁₀ to R₁₉ not being the group represented by the formula (110) are each preferably a hydrogen atom.

In the formula (1000B), L₁₀₀ is preferably a single bond or an arylene group having three or less substituted or unsubstituted benzene rings.

In the formula (1000B), L₁₀₀ is preferably not a substituted or unsubstituted anthrylene group.

In the formula (1000B), L₁₀₀ is also preferably a single bond.

In the formula (1000B), the group represented by -(L₁₀₀)ₘₓ- in the formula (110) is also preferably a group represented by any one of formulae (111) to (120) below.

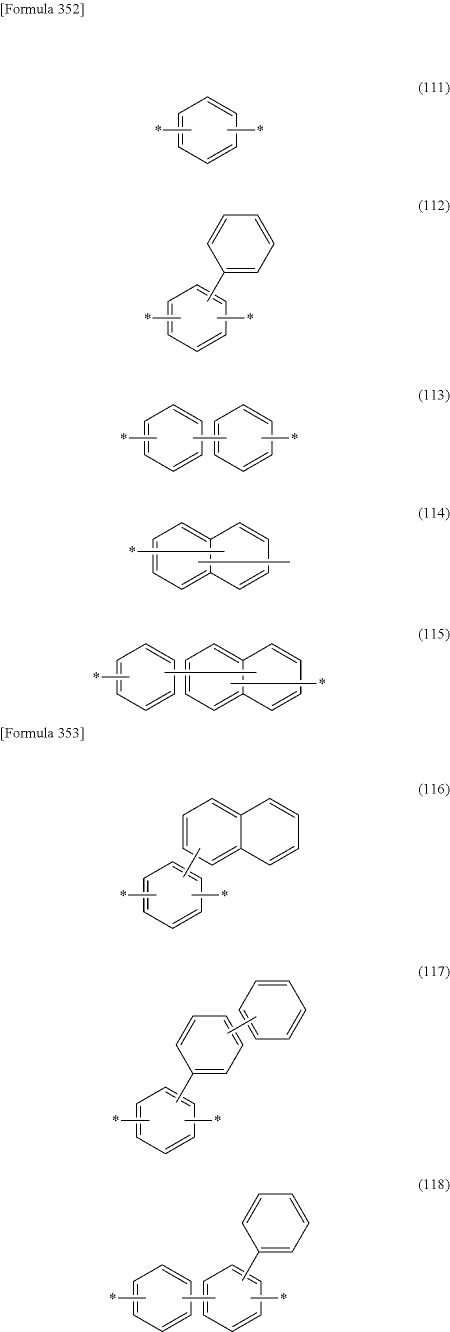

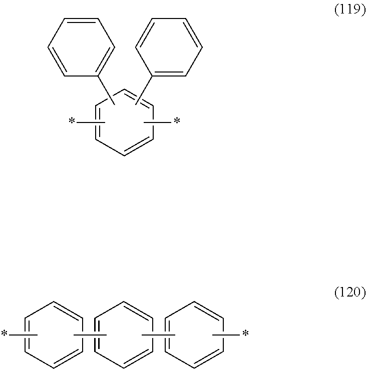

In the formulae (111) to (120), * each represent a bonding position.

The group represented by -(L₁₀₀)ₘₓ- in the formula (110) is preferably a group represented by the formula (111) or (112).

In the formula (1000B), Ar₁₀₀ is preferably an aryl group in which at least four substituted or unsubstituted benzene rings are fused.

In the formula (1000B), Ar₁₀₀ is preferably an aryl group in which four substituted or unsubstituted benzene rings are fused or an aryl group in which five substituted or unsubstituted benzene rings are fused.

In the formula (1000B): Ar₁₀₀ is preferably a group represented by a formula (1100), a formula (1200), a formula (1300), a formula (1400), a formula (1500), a formula (1600), a formula (1700) or a formula (1800) below.

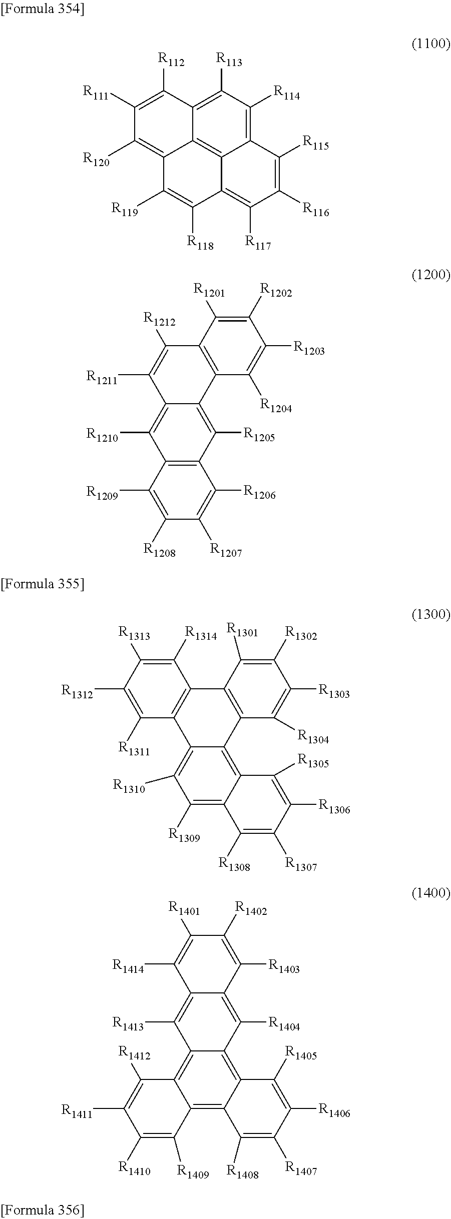

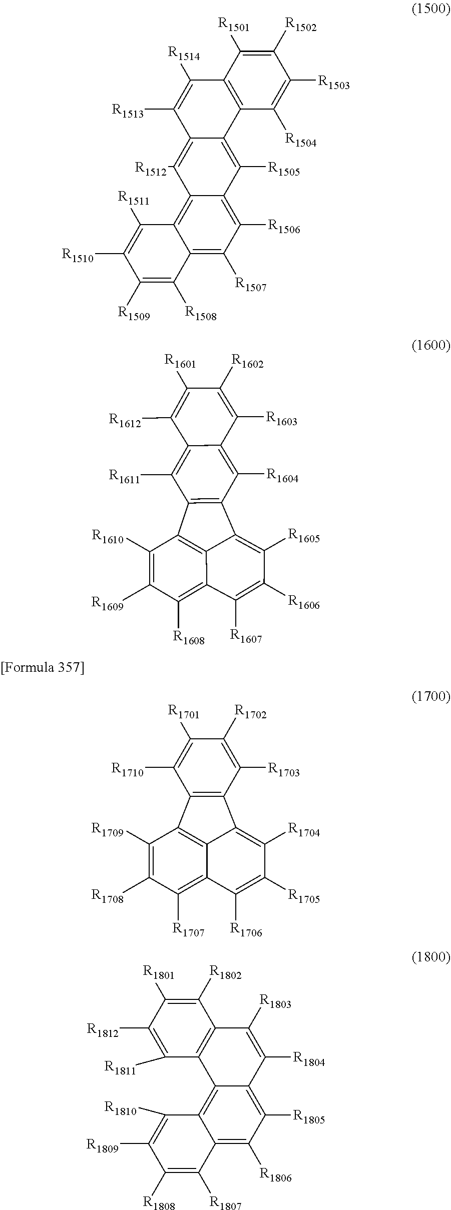

In the formula (1100), one of R₁₁₁ to R₁₂₀ is a bond;
in the formula (1200), one of R₁₂₀₁ to R₁₂₁₂ is a bond;
in the formula (1300), one of R₁₃₀₁ to R₁₃₁₄ is a bond;
in the formula (1400), one of R₁₄₀₁ to R₁₄₁₄ is a bond;
in the formula (1500), one of R₁₅₀₁ to R₁₅₁₄ is a bond;
in the formula (1600), one of R₁₆₀₁ to R₁₆₁₂ is a bond;
in the formula (1700), one of R₁₇₀₁ to R₁₇₁₀ is a bond;
in the formula (1800), one of R₁₈₀₁ to R₁₈₁₂ is a bond; and
R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms.

The group represented by the formula (1100) in which R₁₁₁ is the bond is represented by a formula (1112) below. The group represented by the formula (1100) in which R₁₂₀ is the bond is represented by a formula (1113) below. The group represented by the formula (1100) in which R₁₁₉ is the bond is represented by a formula (1114) below.

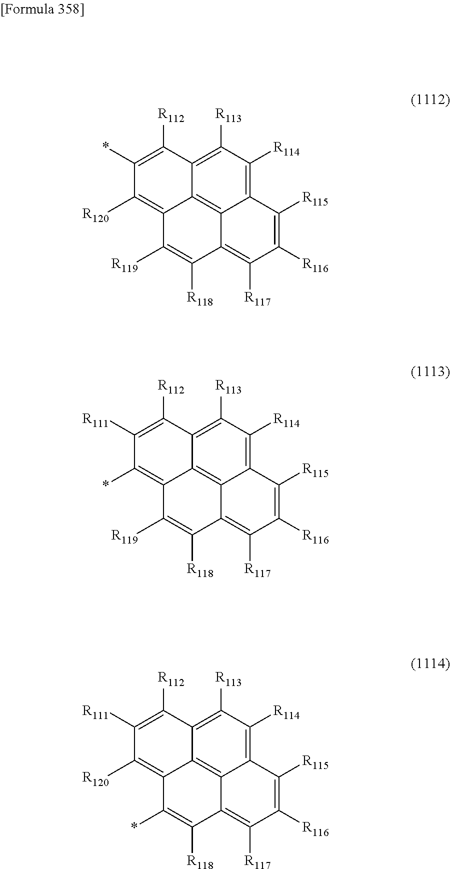

In the formulae (1112), (1113), and (1114):
R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
* in the formulae (1112) to (1114) each represent a bonding position.

In the formulae (1100), (1200), (1300), (1400), (1500), (1600), (1700) and (1800), R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are preferably each independently a hydrogen atom, a substituted or unsubstituted alkyl group having 1 to 50 carbon atoms, a substituted or unsubstituted cycloalkyl group having 3 to 50 ring carbon atoms, or a substituted or unsubstituted aryl group having 6 to 50 ring carbon atoms.

In the formulae (1100), (1200), (1300), (1400), (1500), (1600), (1700) and (1800), R₁₁₁ to R₁₂₀ not being the bond, R₁₂₀₁ to R₁₂₁₂ not being the bond, R₁₃₀₁ to R₁₃₁₄ not being the bond, R₁₄₀₁ to R₁₄₁₄ not being the bond, R₁₅₀₁ to R₁₅₁₄ not being the bond, R₁₆₀₁ to R₁₆₁₂ not being the bond, R₁₇₀₁ to R₁₇₁₀ not being the bond, and R₁₈₀₁ to R₁₈₁₂ not being the bond are preferably each a hydrogen atom.

The compound represented by the formula (1000B) preferably includes only one benzoxanthene ring in a molecule.

The first compound is also preferably a compound obtained by substituting a benzoxanthene ring with a benzothioxanthene ring in a compound represented by each of the formulae (100), (101), and (102).

### Compound Represented by Formula (17X-1)

In an exemplary arrangement of the organic EL device of each exemplary embodiment, the first compound is a compound represented by a formula (17X-1) below.

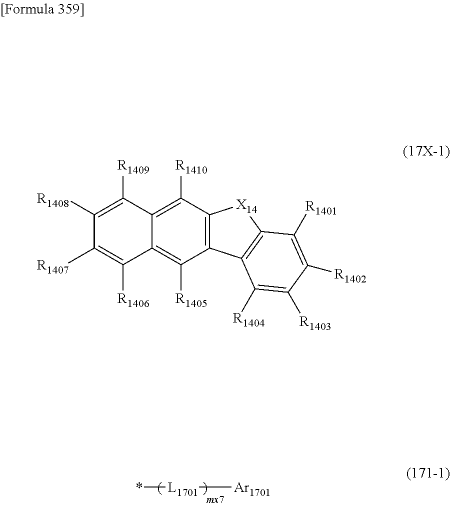

In the formula (17X-1):
X₁₄ is an oxygen atom or a sulfur atom;
at least one combination of adjacent two or more of R₁₄₀₁ to R₁₄₀₄ are mutually bonded to form a substituted or unsubstituted monocyclic ring, mutually bonded to form a substituted or unsubstituted fused ring, or not mutually bonded;
at least one combination of adjacent two or more of R₁₄₀₅ to R₁₄₁₀ are mutually bonded to form a substituted or unsubstituted monocyclic ring, mutually bonded to form a substituted or unsubstituted fused ring, or not mutually bonded;
R₁₄₀₁ to R₁₄₁₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (171-1);
at least one of R₁₄₀₁ to R₁₄₁₀ is a group represented by the formula (171-1);
when a plurality of groups represented by the formula (171-1) are present, the plurality of groups represented by the formula (171-1) are mutually the same or different;
L₁₇₀₁ is a single bond, a substituted or unsubstituted arylene group having 6 to 50 ring carbon atoms, or a substituted or unsubstituted divalent heterocyclic group having 5 to 50 ring atoms;
Ar₁₇₀₁ is a substituted or unsubstituted aryl group having 6 to 50 ring carbon atoms, or a substituted or unsubstituted heterocyclic group having 5 to 50 ring atoms;
mx7 is 0, 1, 2, 3, 4, or 5;
when two or more L₁₇₀₁ are present, the two or more L₁₇₀₁ are mutually the same or different;
when two or more Ar₁₇₀₁ are present, the two or more Ar₁₇₀₁ are mutually the same or different;
R₉₀₁ to R₉₀₅, R₈₀₁ and R₈₀₂ each independently represent the same as R₉₀₁ to R₉₀₅, R₈₀₁ and R₈₀₂ in the formula (1000B); and
* in the formula (171-1) represents a bonding position to a ring represented by the formula (17X-1).

### Compound Represented by Formula (17X-2)

In an exemplary arrangement of the organic EL device of each exemplary embodiment, the first compound is a compound represented by a formula (17X-2) below.

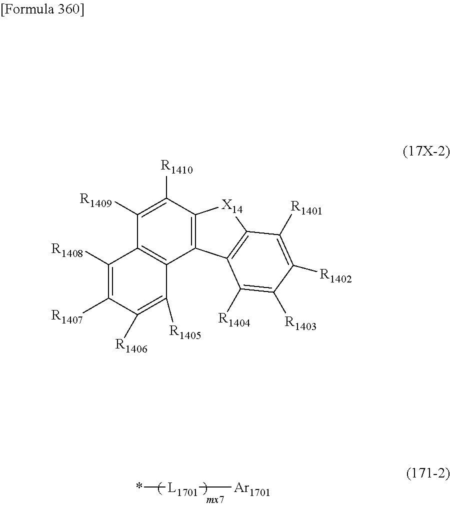

In the formula (17X-2):
R₁₄₀₁ to R₁₄₁₀ and X₁₄ each independently represent the same as R₁₄₀₁ to R₁₄₁₀ and X₁₄ in the formula (17X-1);
the group represented by the formula (171-2) represents the same as a group represented by the formula (171-1); and L₁₇₀₁, Ar₁₇₀₁ and mx7 in the formula (171-2) each independently represent the same as L₁₇₀₁, Ar₁₇₀₁ and mx7 in the formula (171-1);
when a plurality of groups represented by the formula (171-2) are present, the plurality of groups represented by the formula (171-2) are mutually the same or different; and
* in the formula (171-2) represents a bonding position to a ring represented by the formula (17X-2).

Compound Represented by Formula (17X-3) In an exemplary arrangement of the organic EL device of each exemplary embodiment, the first compound is a compound represented by a formula (17X-3) below.

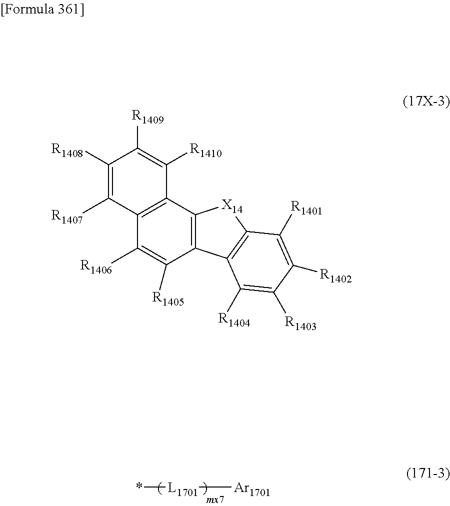

In the formula (17X-3):
R₁₄₀₁ to R₁₄₁₀ and X₁₄ each independently represent the same as R₁₄₀₁ to R₁₄₁₀ and X₁₄ in the formula (17X-1);
the group represented by the formula (171-3) represents the same as a group represented by the formula (171-1); and L₁₇₀₁, Ar₁₇₀₁ and mx7 in the formula (171-3) each independently represent the same as L₁₇₀₁, Ar₁₇₀₁ and mx7 in the formula (171-1);
when a plurality of groups represented by the formula (171-3) are present, the plurality of groups represented by the formula (171-3) are mutually the same or different; and
* in the formula (171-3) represents a bonding position to a ring represented by the formula (17X-3).

In the formulae (17X-1), (17X-2) and (17X-3), X₁₄ is preferably an oxygen atom.

### Compound Represented by Formula (18)

In an exemplary arrangement of the organic EL device of each exemplary embodiment, the first compound is a compound represented by the formula (18) below.

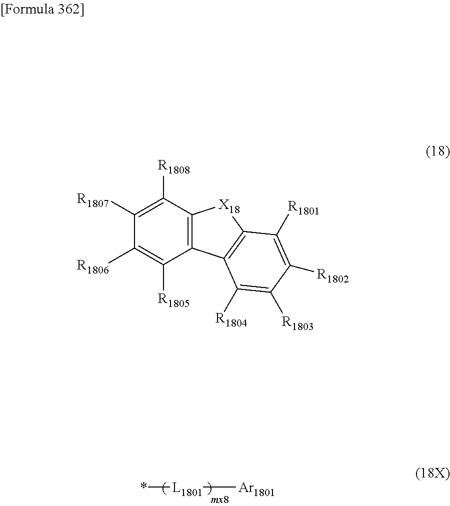

In the formula (18):
X₁₈ is an oxygen atom or a sulfur atom;
at least one combination of adjacent two or more of R₁₈₀₁ to R₁₈₀₄ are mutually bonded to form a substituted or unsubstituted monocyclic ring, mutually bonded to form a substituted or unsubstituted fused ring, or not mutually bonded;
at least one combination of adjacent two or more of R₁₈₀₅ to R₁₈₀₈ are mutually bonded to form a substituted or unsubstituted monocyclic ring, mutually bonded to form a substituted or unsubstituted fused ring, or not mutually bonded;
R₁₈₀₁ to R₁₈₀₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (18X);
at least one of R₁₈₀₁ to R₁₈₀₈ is a group represented by the formula (18X);
when a plurality of groups represented by the formula (18X) are present, the plurality of groups represented by the formula (18X) are mutually the same or different;
L₁₈₀₁ is a single bond, a substituted or unsubstituted arylene group having 6 to 50 ring carbon atoms, or a substituted or unsubstituted divalent heterocyclic group having 5 to 50 ring atoms;
Ar₁₈₀₁ is a substituted or unsubstituted aryl group having 6 to 50 ring carbon atoms, or a substituted or unsubstituted heterocyclic group having 5 to 50 ring atoms;
mx8 is 0, 1, 2, 3, 4, or 5;
when two or more L₁₈₀₁ are present, the two or more L₁₈₀₁ are mutually the same or different;
when two or more Ar₁₈₀₁ are present, the two or more Ar₁₈₀₁ are mutually the same or different;
R₉₀₁ to R₉₀₅, R₈₀₁ and R₈₀₂ each independently represent the same as R₉₀₁ to R₉₀₅, R₈₀₁ and R₈₀₂ in the formula (1000B); and
* in the formula (18X) represents a bonding position to a ring represented by the formula (18).

In the formula (18), X₁₈ is preferably an oxygen atom.

In the first compound, the groups specified to be “substituted or unsubstituted” are each preferably an “unsubstituted” group.

In the organic EL device of each exemplary embodiment, also preferably, the second host material has, in a molecule, a linking structure including a benzene ring and a naphthalene ring linked to each other with a single bond, in which the benzene ring and the naphthalene ring in the linking structure are each independently fused or not fused with a further monocyclic ring or fused ring, and the benzene ring and the naphthalene ring in the linking structure are further linked to each other by cross-linking at at least one site other than the single bond.

When the second host material has the linking structure including such cross-linking, deterioration in chromaticity of the organic EL device is expected to be inhibited.

The second host material in the above case is only required to have a linking structure as the minimum unit in a molecule, the linking structure including a benzene ring and a naphthalene ring linked to each other with a single bond (occasionally referred to as a benzene-naphthalene linking structure), the linking structure being as represented by a formula (X1) or a formula (X2) below. Further, the benzene ring may be fused with a monocyclic ring or fused ring, and the naphthalene ring may be fused with a monocyclic ring or fused ring. For instance, also in a case where the second host material has, in a molecule, a linking structure including a naphthalene ring and a naphthalene ring linked to each other with a single bond (occasionally referred to as a naphthalene-naphthalene linking structure) and being as represented by a formula (X3), a formula (X4), or a formula (X5) below, the naphthalene-naphthalene linking structure is regarded as including the benzene-naphthalene linking structure since one of the naphthalene rings includes a benzene ring.

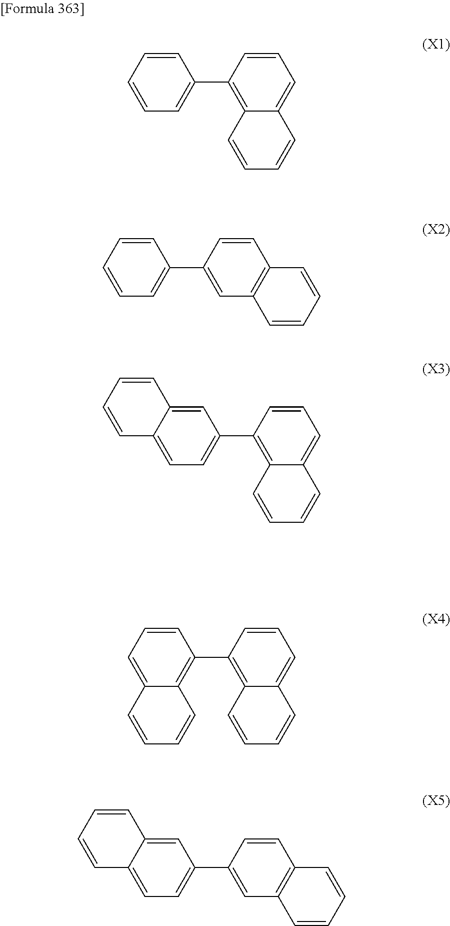

In the organic EL device of each exemplary embodiment, the cross-linking also preferably includes a double bond. Specifically, the second host material also preferably has a structure in which the benzene ring and the naphthalene ring are further linked to each other at any other site than the single bond by the cross-linking structure including a double bond.

Assuming that the benzene ring and the naphthalene ring in the benzene-naphthalene linking structure are further linked to each other at at least one site other than the single bond by cross-linking, for instance, a linking structure (fused ring) represented by a formula (X11) below is obtained in a case of the formula (X1), and a linking structure (fused ring) represented by a formula (X31) below is obtained in a case of the formula (X3).

Assuming that the benzene ring and the naphthalene ring in the benzene-naphthalene linking structure are further linked to each other at any other site than the single bond by cross-linking including a double bond, for instance, a linking structure (fused ring) represented by a formula (X12) below is obtained in a case of the formula (X1), a linking structure (fused ring) represented by a formula (X21) or formula (X22) below is obtained in a case of the formula (X2), a linking structure (fused ring) represented by a formula (X41) below is obtained in a case of the formula (X4), and a linking structure (fused ring) represented by a formula (X51) below is obtained in a case of the formula (X5).

Assuming that the benzene ring and the naphthalene ring in the benzene-naphthalene linking structure are further linked to each other at at least one site other than the single bond by cross-linking including a hetero atom (e.g., an oxygen atom), for instance, a linking structure (fused ring) represented by a formula (X13) below is obtained in a case of the formula (X1).

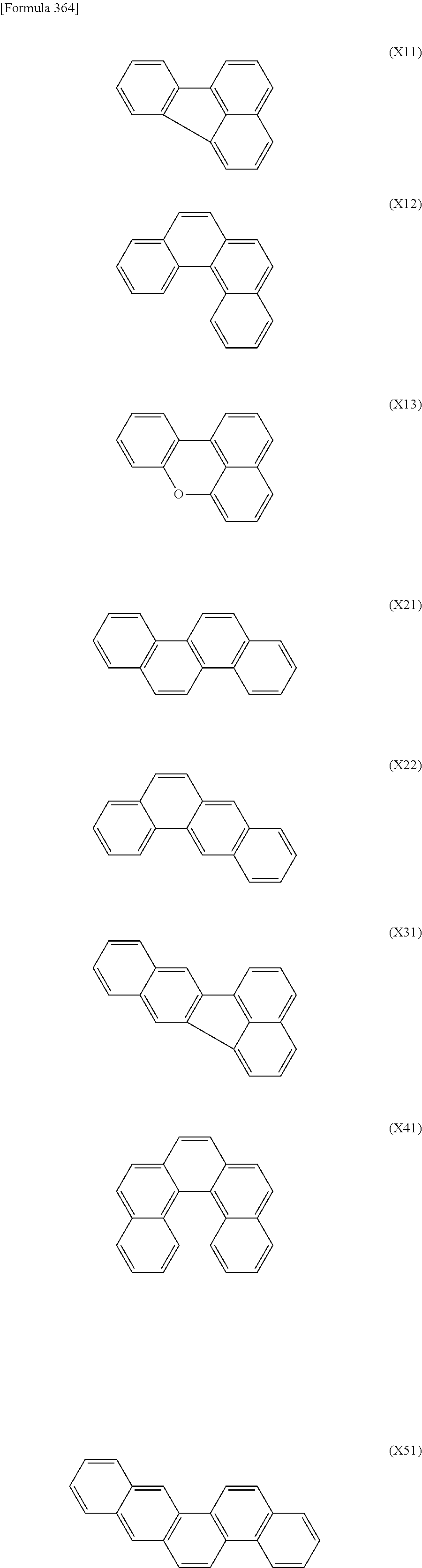

In the organic EL device of each exemplary embodiment, also preferably, the second host material has, in a molecule, a biphenyl structure including a first benzene ring and a second benzene ring linked to each other with a single bond, and the first benzene ring and the second benzene ring in the biphenyl structure are further linked to each other by cross-linking at at least one site other than the single bond.

In the organic EL device of each exemplary embodiment, also preferably, the first benzene ring and the second benzene ring in the biphenyl structure are further linked to each other by the cross-linking at one site other than the single bond. When the second host material has the biphenyl structure including such cross-linking, deterioration in chromaticity of the organic EL device is expected to be inhibited.

In the organic EL device of each exemplary embodiment, the cross-linking also preferably includes a double bond.

In the organic EL device of each exemplary embodiment, the cross-linking also preferably includes no double bond.

Also preferably, the first benzene ring and the second benzene ring in the biphenyl structure are further linked to each other by the cross-linking at two sites other than the single bond.

In the organic EL device of each exemplary embodiment, also preferably, the first benzene ring and the second benzene ring in the biphenyl structure are further linked to each other by the cross-linking at two sites other than the single bond, and the cross-linking includes no double bond. When the second host material has the biphenyl structure including such cross-linking, deterioration in chromaticity of the organic EL device is expected to be inhibited.

For instance, assuming that the first benzene ring and the second benzene ring in the biphenyl structure represented by a formula (BP1) below are further linked to each other by cross-linking at at least one site other than the single bond, the biphenyl structure is exemplified by linking structures (fused rings) represented by formulae (BP11) to (BP15) below.

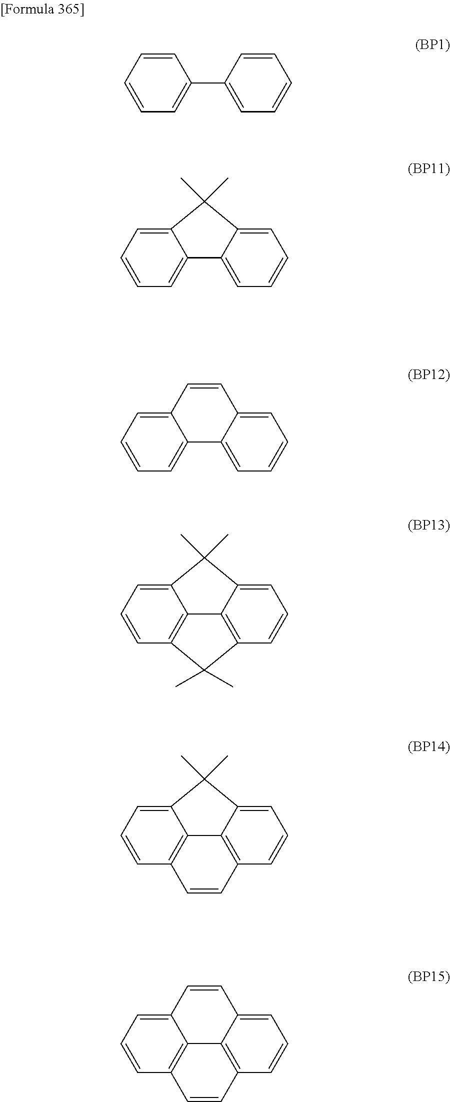

The formula (BP11) represents a linking structure in which the first benzene ring and the second benzene ring are linked to each other at one site other than the single bond by cross-linking including no double bond.

The formula (BP12) represents a linking structure in which the first benzene ring and the second benzene ring are linked to each other at one site other than the single bond by cross-linking including a double bond.

The formula (BP13) represents a linking structure in which the first benzene ring and the second benzene ring are linked to each other at two sites other than the single bond by cross-linking including no double bond.

The formula (BP14) represents a linking structure in which the first benzene ring and the second benzene ring are linked to each other by cross-linking including no double bond at one of two sites other than the single bond, and the first benzene ring and the second benzene ring are linked to each other by cross-linking including a double bond at the other of the two sites other than the single bond.

The formula (BP15) represents a linking structure in which the first benzene ring and the second benzene ring are linked to each other at two sites other than the single bond by cross-linking including a double bond.

In the first compound, the groups specified to be “substituted or unsubstituted” are each preferably an “unsubstituted” group.

### Method of Producing First Compound

The first compound usable for the organic EL device of each exemplary embodiment can be produced by a known method. The first compound can also be produced based on a known method through a known alternative reaction using a known material(s) tailored for the target compound.

### Specific Examples of First Compound

Specific examples of the first compound usable for the organic EL device of each exemplary embodiment include compounds below. It should however be noted that the invention is not limited to the specific examples of the first compound.

In the specific examples of the compound herein, D represents a deuterium atom, Me represents a methyl group, and tBu represents a tert-butyl group.

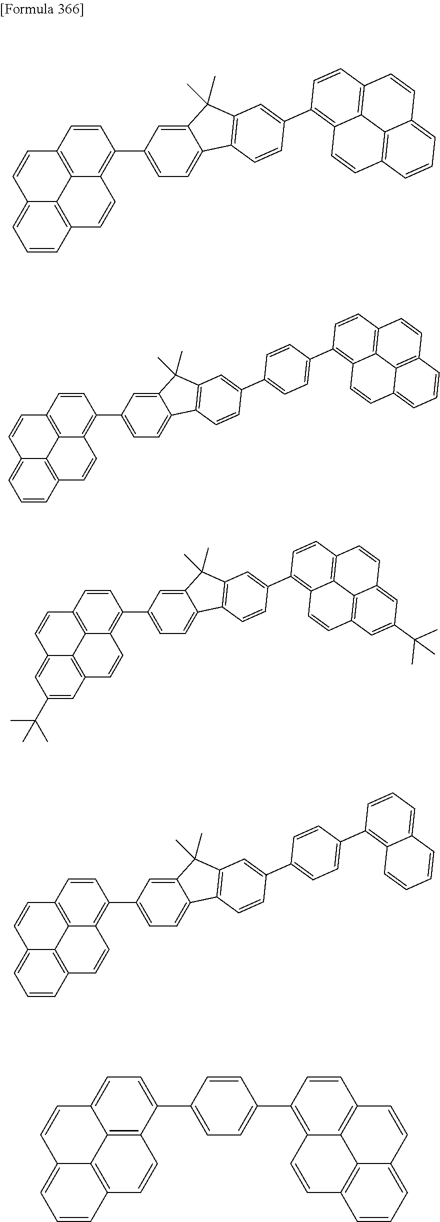

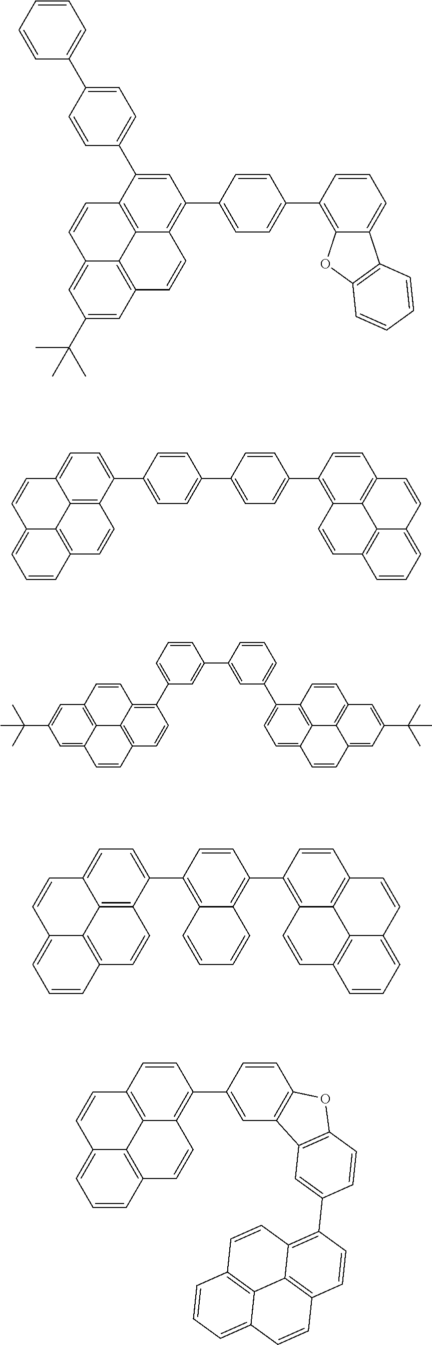

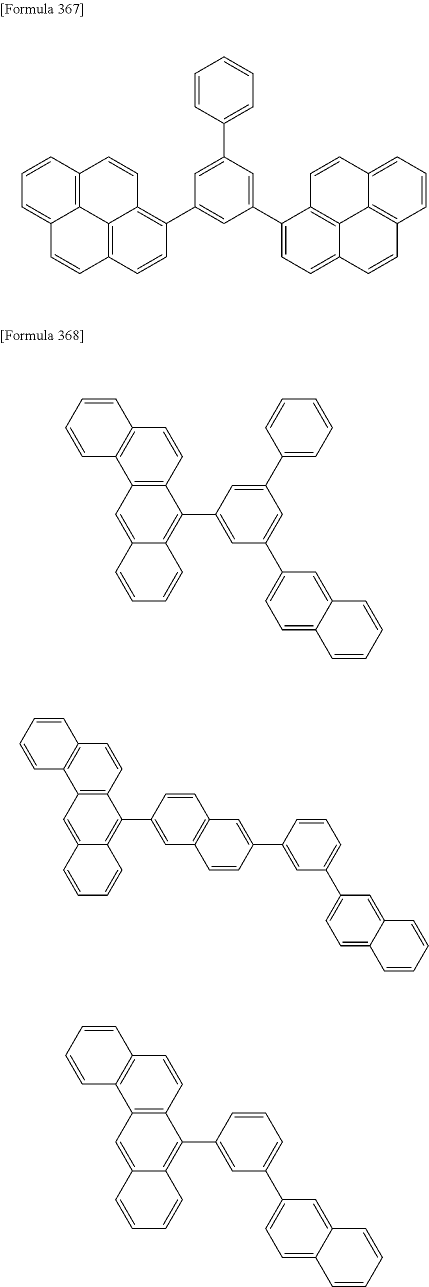

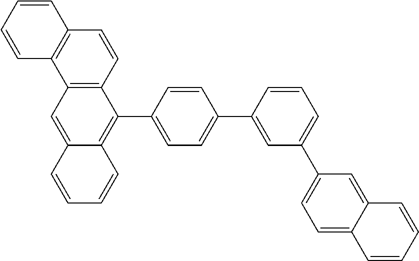 [SMILES: c1cc(-c2ccc(-c3c4ccccc4cc4c3ccc3ccccc34)cc2)cc(-c2ccc3ccccc3c2)c1]

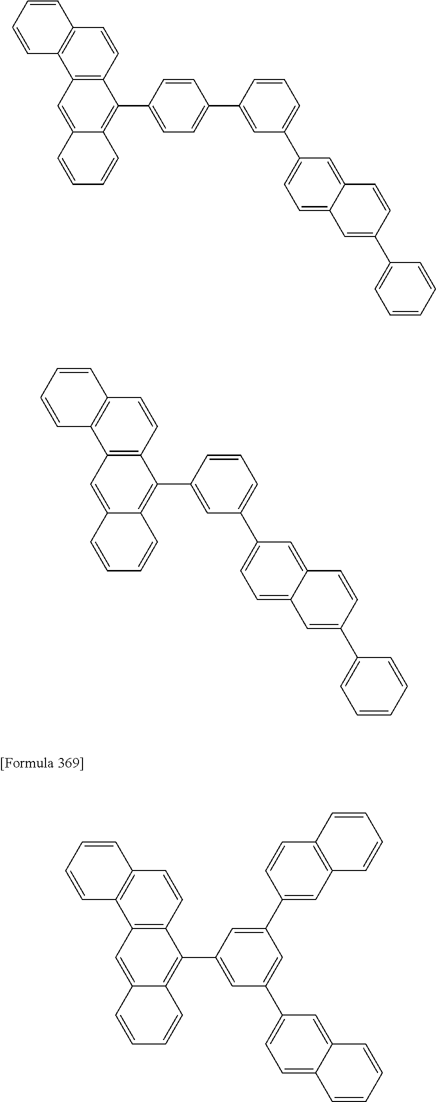

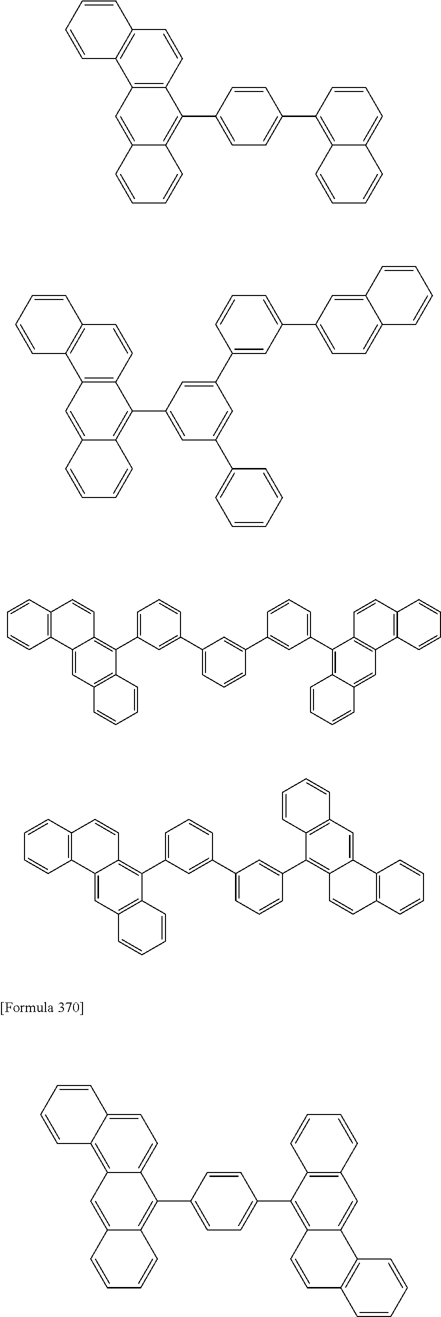

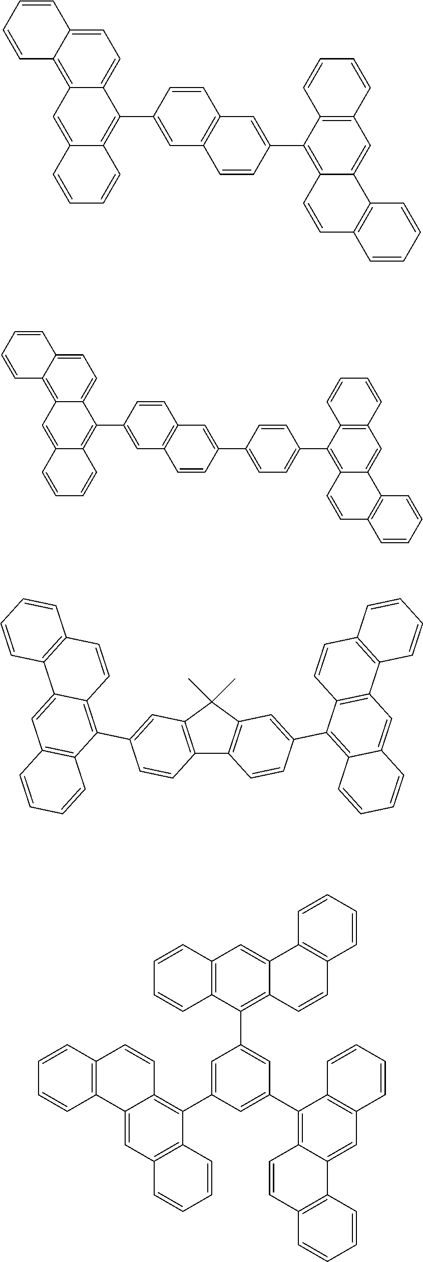

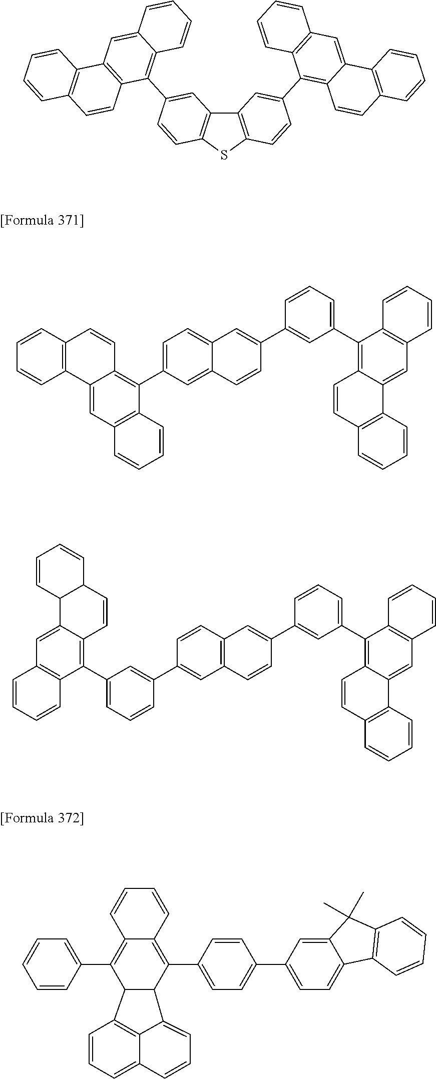

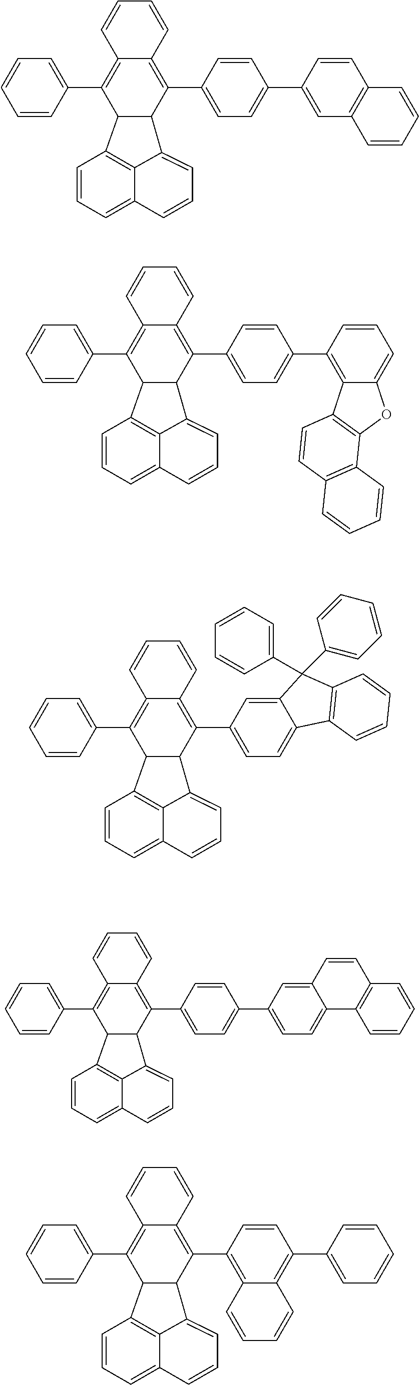

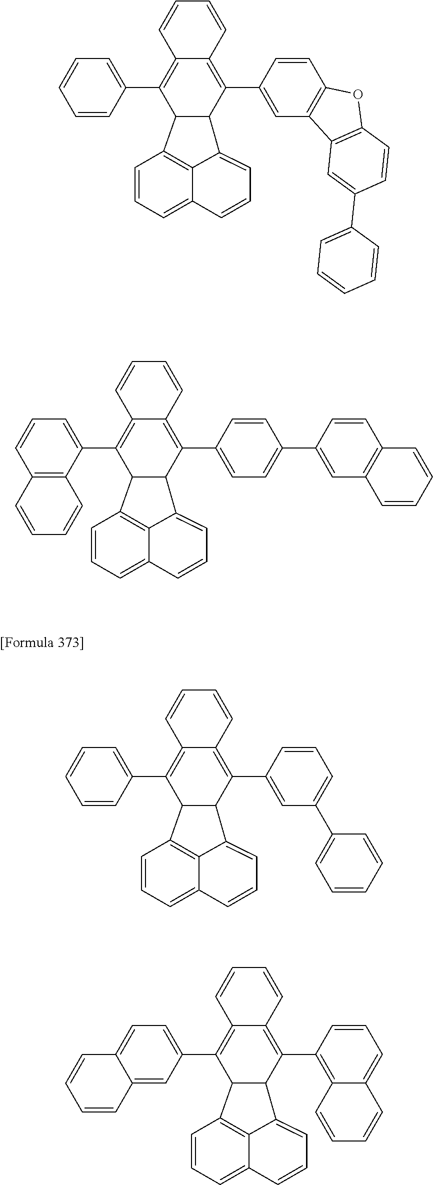

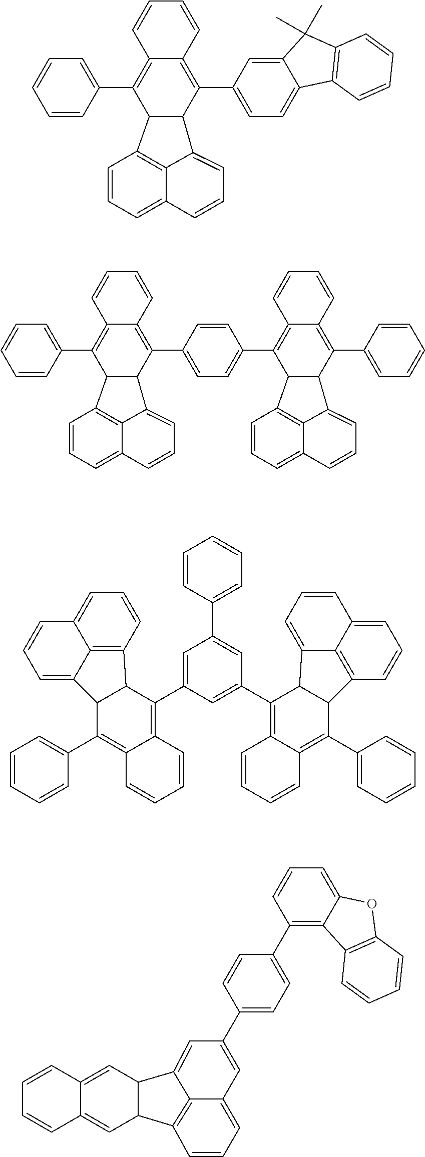

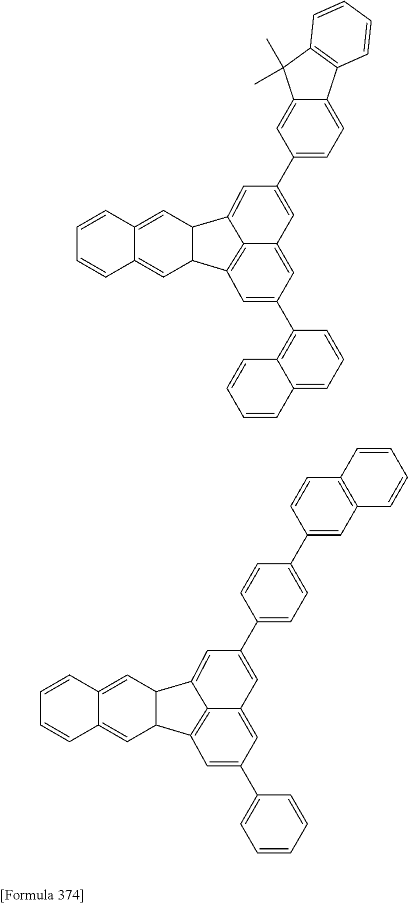

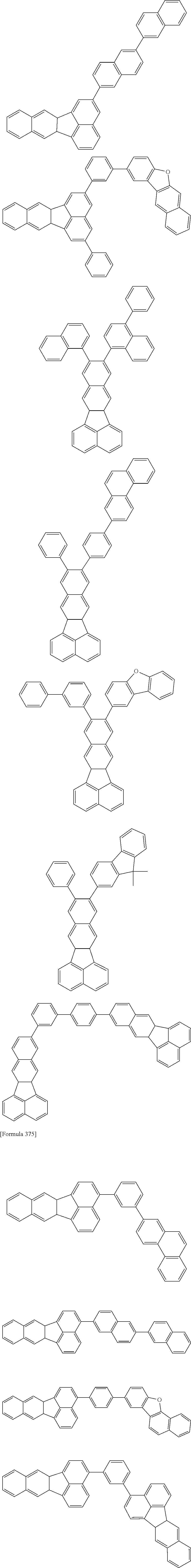

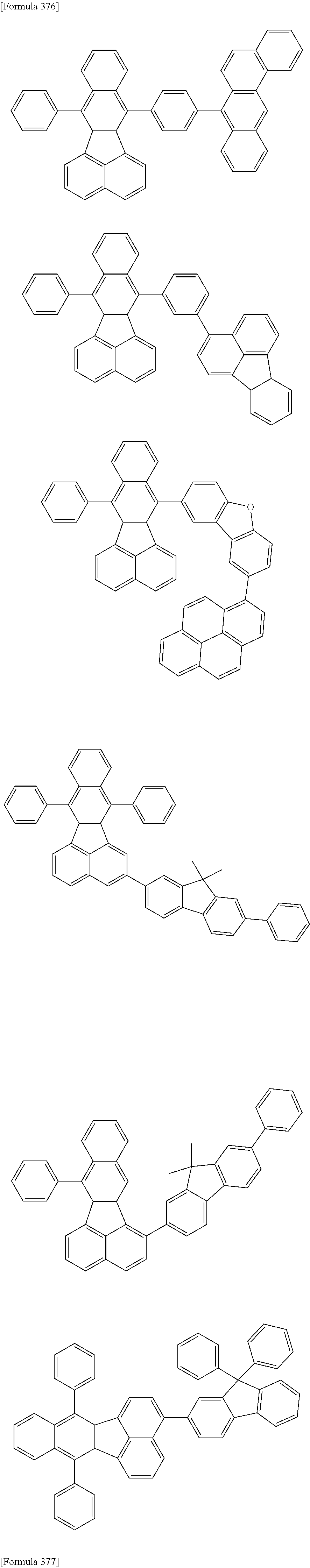

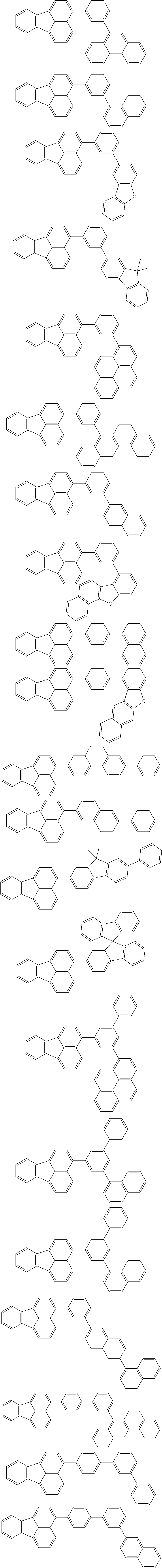

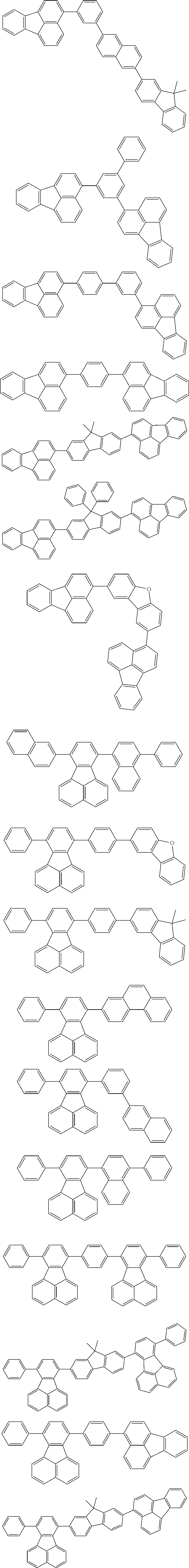

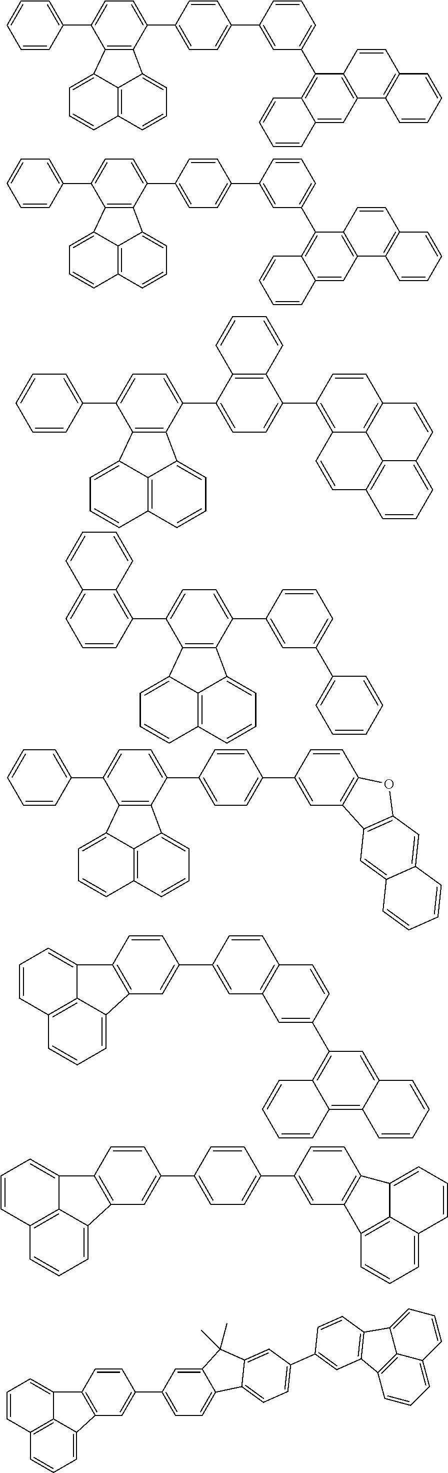

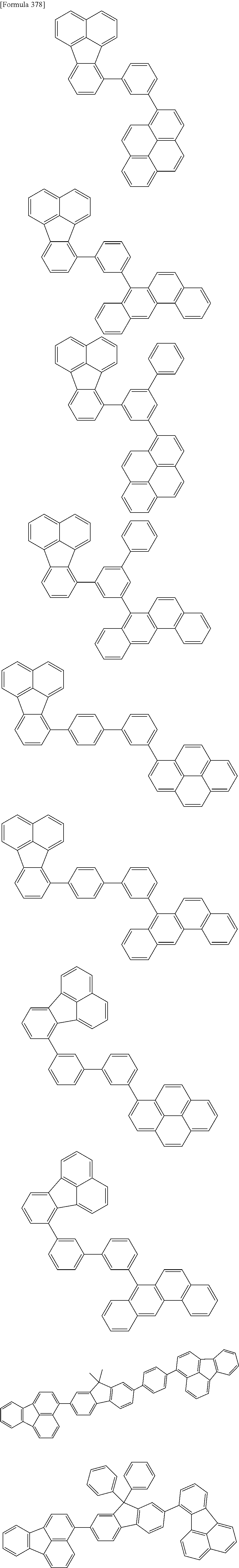

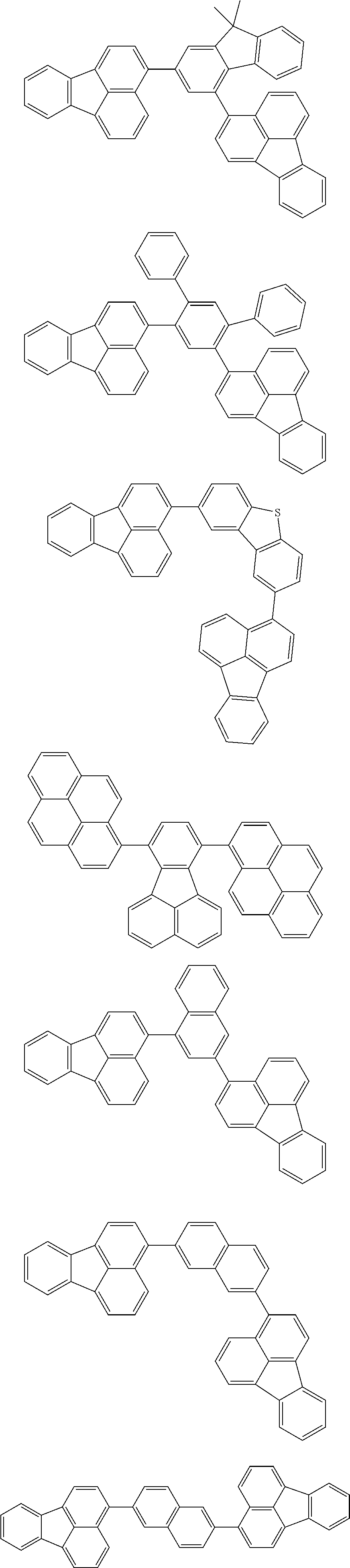

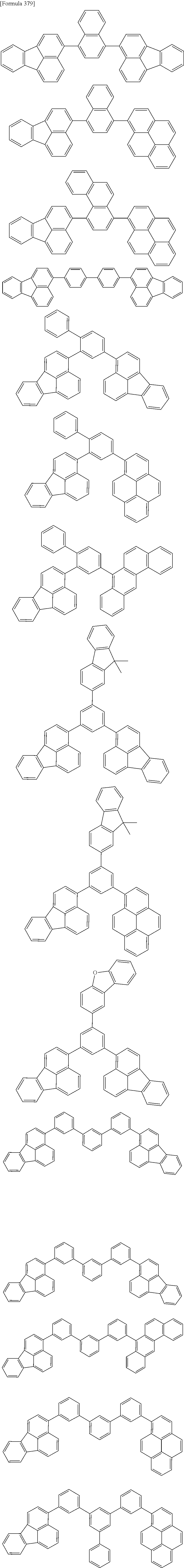

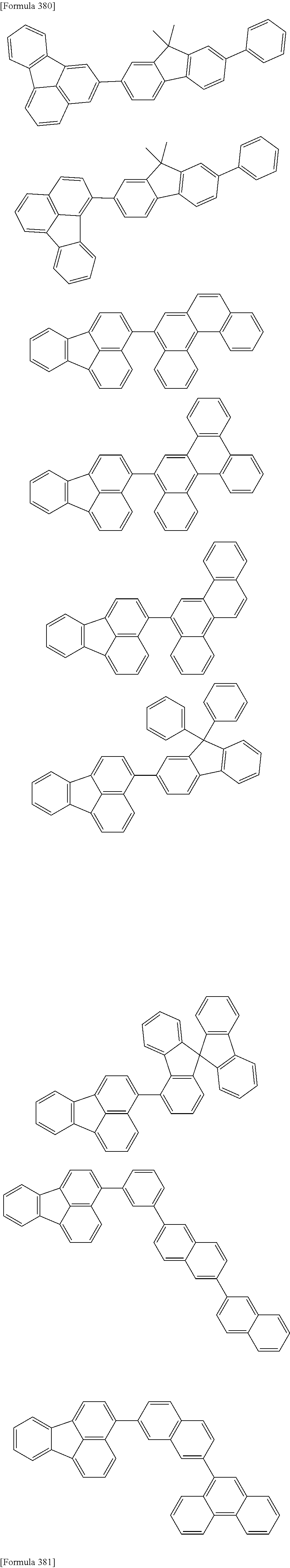

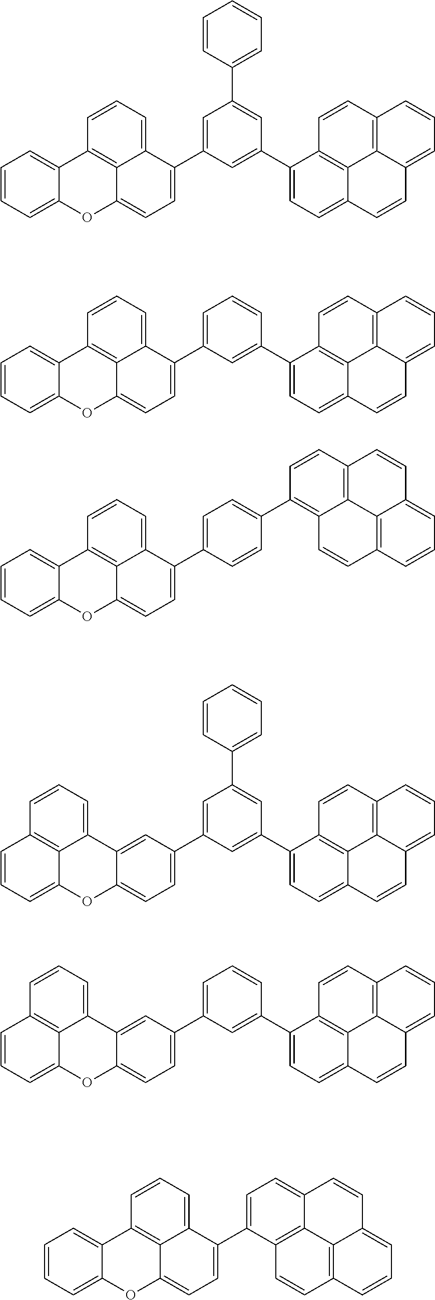

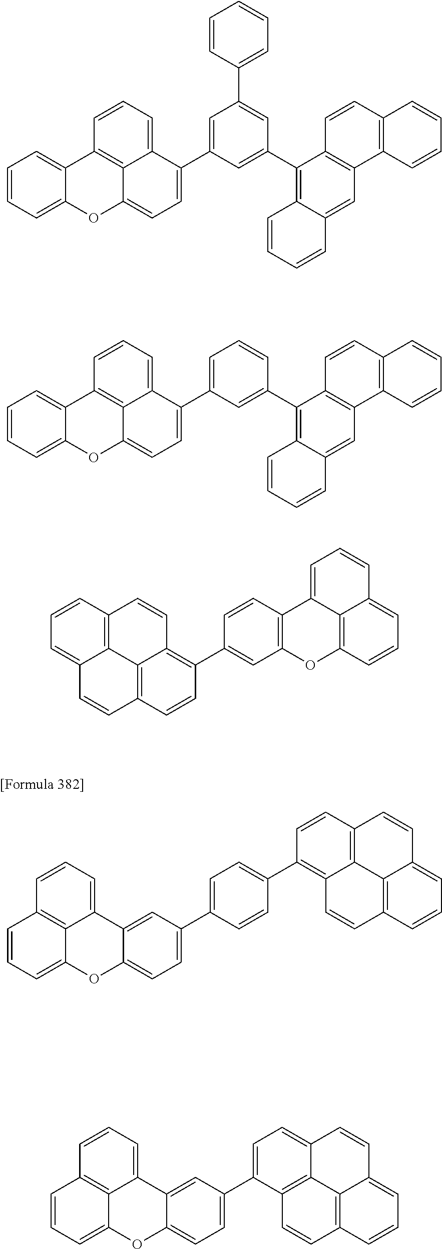 [SMILES: c1ccc(-c2cc(-c3ccc4c5c(cccc35)-c3ccccc3O4)cc(-c3c4ccccc4cc4c3ccc3ccccc34)c2)cc1 ; c1cc(-c2ccc3c4c(cccc24)-c2ccccc2O3)cc(-c2c3ccccc3cc3c2ccc2ccccc23)c1 ; c1cc2c3c(cccc3c1)-c1ccc(-c3ccc4ccc5cccc6ccc3c4c56)cc1O2 ; c1cc2c3c(cccc3c1)-c1cc(-c3ccc(-c4ccc5ccc6cccc7ccc4c5c67)cc3)ccc1O2 ; c1cc2c3c(cccc3c1)-c1cc(-c3ccc4ccc5cccc6ccc3c4c56)ccc1O2]

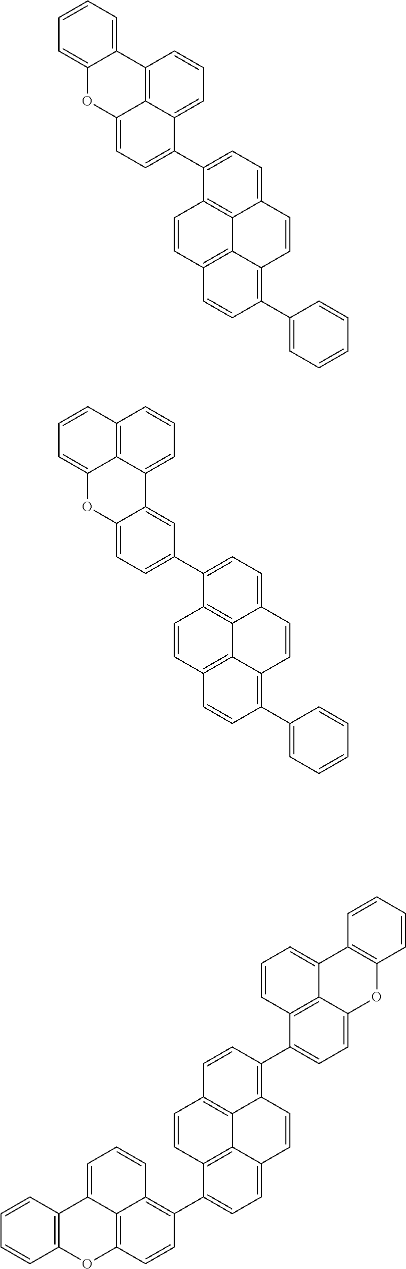 [SMILES: c1ccc(-c2ccc3ccc4c(-c5ccc6c7c(cccc57)-c5ccccc5O6)ccc5ccc2c3c54)cc1 ; c1ccc(-c2ccc3ccc4c(-c5ccc6c(c5)-c5cccc7cccc(c57)O6)ccc5ccc2c3c54)cc1 ; c1ccc2c(c1)Oc1ccc(-c3ccc4ccc5c(-c6ccc7c8c(cccc68)-c6ccccc6O7)ccc6ccc3c4c65)c3cccc-2c13]

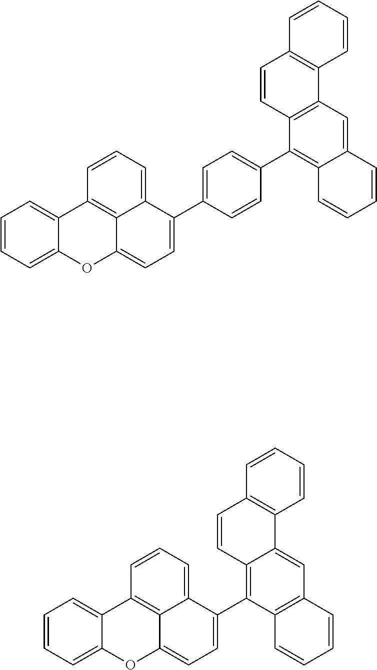 [SMILES: c1ccc2c(c1)Oc1ccc(-c3ccc(-c4c5ccccc5cc5c4ccc4ccccc45)cc3)c3cccc-2c13 ; c1ccc2c(c1)Oc1ccc(-c3c4ccccc4cc4c3ccc3ccccc34)c3cccc-2c13]

### Luminescent Compound

In an exemplary arrangement of the organic EL device of each exemplary embodiment, the luminescent compound is at least one compound selected from the group consisting of a compound represented by a formula (3) below, a compound represented by a formula (4) below, a compound represented by a formula (5) below, a compound represented by a formula (6) below, a compound represented by a formula (7) below, a compound represented by a formula (8) below, a compound represented by a formula (9) below, and a compound represented by a formula (10) below.

In an exemplary arrangement of the organic EL device of each exemplary embodiment, a luminescent compound contained in the first emitting layer is a first luminescent compound and a luminescent compound contained in the second emitting layer is a second luminescent compound. Similar to the description regarding the luminescent compound, the first luminescent compound and the second luminescent compound are also each independently at least one compound selected from the group consisting of compounds represented by the formulae (3) to (10) below.

In an exemplary arrangement of the organic EL device of each exemplary embodiment, the luminescent compound is a compound represented by the formula (6) below.

### Compound Represented by Formula (6)

A compound represented by the formula (6) will be described below.

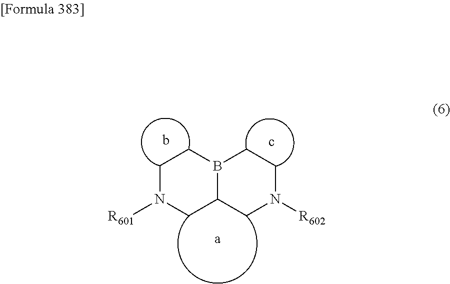

In the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, ring b or ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device of each exemplary embodiment, the ring a, ring b and ring c are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the rings a, b, and c has the same structure as a compound formed by introducing a hydrogen atom to the “aryl group”.

Ring atoms of the “aromatic hydrocarbon ring” for the ring a include three carbon atoms on the fused bicyclic structure at the center of the formula (6).

Ring atoms of the “aromatic hydrocarbon ring” for the rings b and c include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the rings a, b, and c has the same structure as a compound formed by introducing a hydrogen atom to the “heterocyclic group” described above.

Ring atoms of the “heterocycle” for the ring a include three carbon atoms on the fused bicyclic structure at the center of the formula (6). Ring atoms of the “heterocycle” for the rings b and c include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ may be each independently bonded with the ring a, ring b, or ring c to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ being bonded with the ring a, ring b, or ring c specifically means that atoms forming R₆₀₁ and R₆₀₂ are bonded with atoms forming the ring a, ring b, or ring c. For instance, R₆₀₁ may be bonded with the ring a to form a bicyclic (or tri-or-more cyclic) fused nitrogen-containing heterocycle, in which the ring including R₆₀₁ and the ring a are fused. Specific examples of the nitrogen-containing heterocycle include a compound corresponding to the nitrogen-containing bi (or-more) cyclic fused heterocyclic group in the specific example group G2.

The same applies to R₆₀₁ bonded with the ring b, R₆₀₂ bonded with the ring a, and R₆₀₂ bonded with the ring c.

Optionally, R₆₀₁ and R₆₀₂ are each independently not bonded with the ring a, ring b, or ring c.

In an exemplary embodiment, the ring a, ring b and ring c in the formula (6) are each independently a substituted or unsubstituted aromatic hydrocarbon ring having 6 to 50 ring carbon atoms.

In an exemplary embodiment, the ring a, ring b and ring c in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms or a substituted or unsubstituted heterocyclic group having 5 to 50 ring atoms, preferably a substituted or unsubstituted aryl group having 6 to 50 ring carbon atoms.

In an exemplary embodiment, the compound represented by the formula (6) is a compound represented by a formula (62) below.

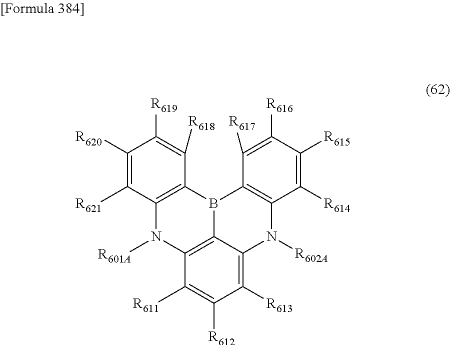

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (62), R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the ring a are fused. Specific examples of the nitrogen-containing heterocycle include a compound corresponding to the nitrogen-containing bi (or-more) cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ may be mutually bonded to form a substituted or unsubstituted monocyclic ring or a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom or a substituted or unsubstituted alkyl group having 1 to 50 carbon atoms.

In an exemplary embodiment, R₆₁₁ to R₆₂₁ not contributing to ring formation are each independently a hydrogen atom or a substituted or unsubstituted alkyl group having 1 to 50 carbon atoms, and at least one of R₆₁₁ to R₆₂₁ is a substituted or unsubstituted alkyl group having 1 to 50 carbon atoms.

In an exemplary embodiment, the compound represented by the formula (62) is a compound represented by a formula (63) below.

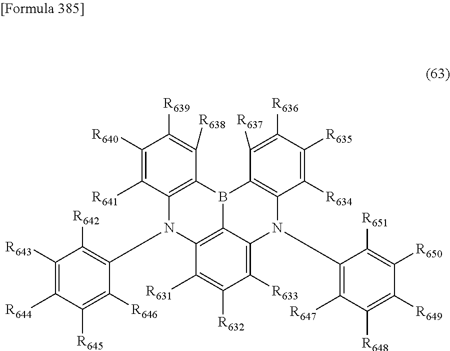

In the formula (63):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and
R₆₃₁ to R₆₅₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ is optionally bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the ring a are fused. Specific examples of the nitrogen-containing heterocycle include a compound corresponding to a nitrogen-containing tri (-or-more) cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom or a substituted or unsubstituted alkyl group having 1 to 50 carbon atoms.

In an exemplary embodiment, R₆₃₁ to R₆₅₁ not contributing to ring formation are each independently a hydrogen atom or a substituted or unsubstituted alkyl group having 1 to 50 carbon atoms, and at least one of R₆₃₁ to R₆₅₁ is a substituted or unsubstituted alkyl group having 1 to 50 carbon atoms.

In an exemplary embodiment, the compound represented by the formula (63) is a compound represented by a formula (63A) below.

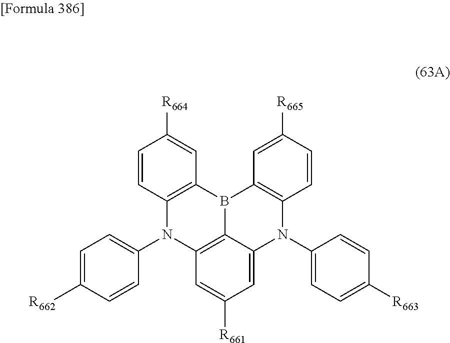

In the formula (63A):
R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is a compound represented by a formula (63B) below.

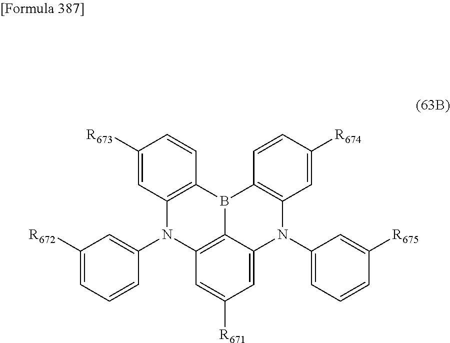

In the formula (63B):
R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63B′) below.

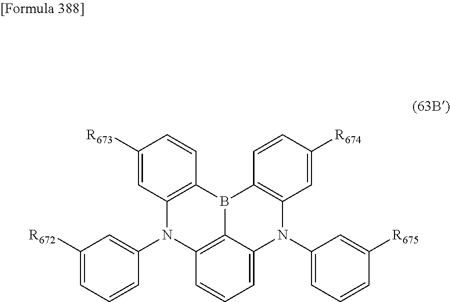

In the formula (63B′), R₆₇₂ to R₆₇₅ each independently represent the same as R₆₇₂ to R₆₇₅ in the formula (63B).

In an exemplary arrangement, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary arrangement: R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and R₆₇₁ and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63C) below.

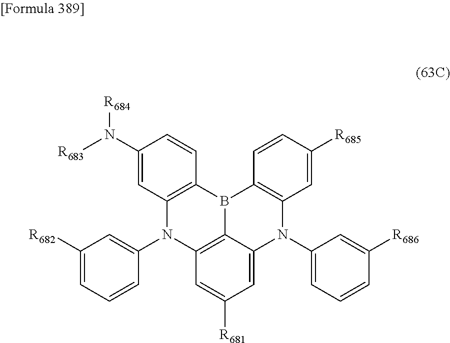

In the formula (63C):
R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is a compound represented by a formula (63C′) below.

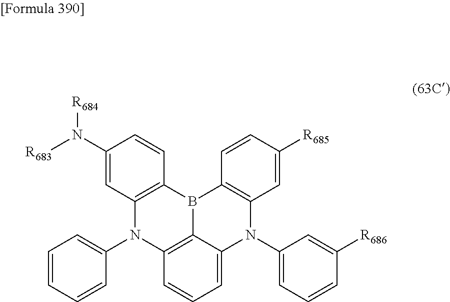

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms or a substituted or unsubstituted aryl group having 6 to 50 ring carbon atoms.

In an exemplary embodiment, R₆₈₁ to R₆₈₆ are each independently a substituted or unsubstituted aryl group having 6 to 50 carbon atoms.

The compound represented by the formula (6) is producible by initially bonding the ring a, ring b and ring c with linking groups (a group including N—R₆₀₁ and a group including N—R₆₀₂) to form an intermediate (first reaction), and bonding the ring a, ring b and ring c with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

In an exemplary embodiment, the compound represented by the formula (6) is a compound represented by a formula (42-2) below.

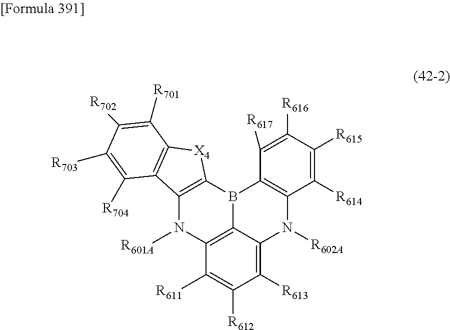

In the formula (42-2):
R₆₁₁ to R₆₁₇, R_{601A} and R_{602A} each independently represent the same as R₆₁₁ to R₆₁₇, R_{601A} and R_{602A} in the formula (62);
X₄ is an oxygen atom or a sulfur atom;
at least one combination of adjacent two or more of R₇₀₁ to R₇₀₄ are mutually bonded to form a substituted or unsubstituted monocyclic ring, mutually bonded to form a substituted or unsubstituted fused ring, or not mutually bonded;
R₇₀₁ to R₇₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆, and R₉₀₇ in the formula (42-2) each independently represent the same as R₉₀₁, R₉₀₂, R₉₀₃, R₉₀₄, R₉₀₅, R₉₀₆, and R₉₀₇ in the formula (62).

### Specific Examples of Compound Represented by Formula (6)

Specific examples of the compound represented by the formula (6) are given below. It should however be noted that these specific examples are merely exemplary and do not limit the compound represented by the formula (6).

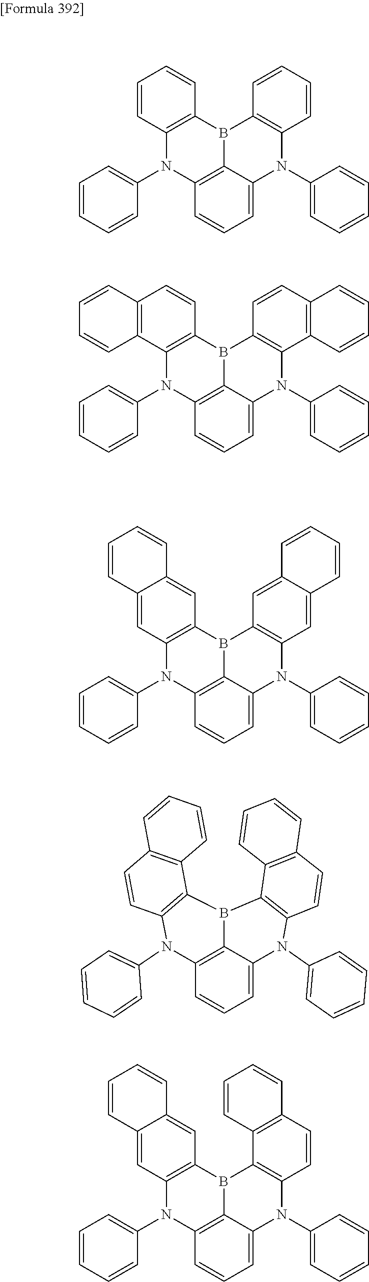 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

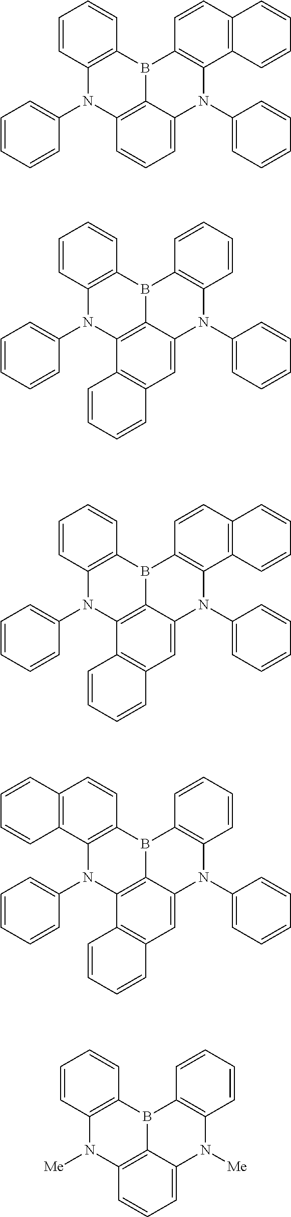 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

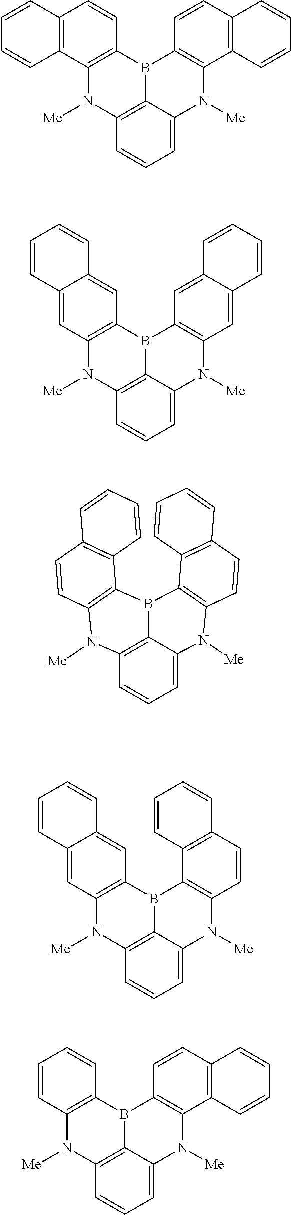 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

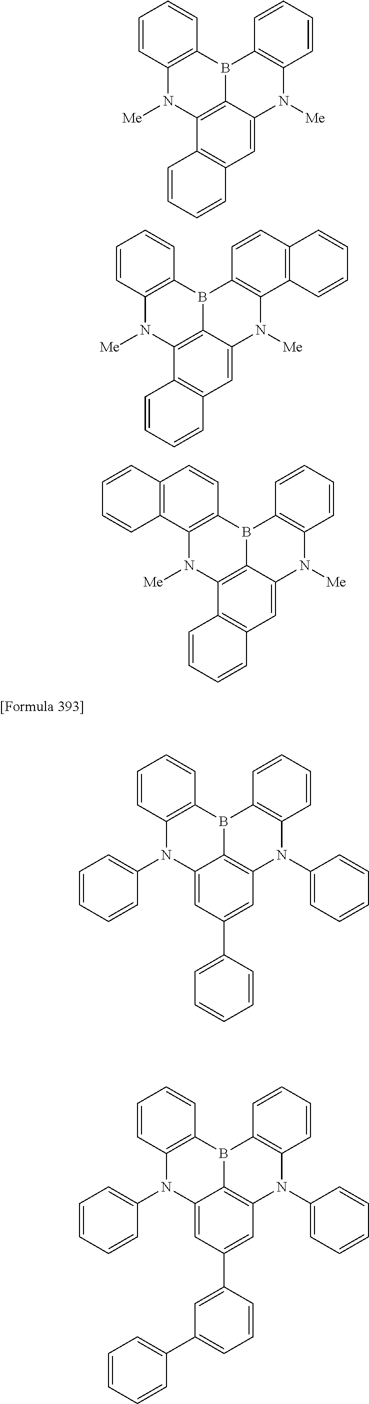

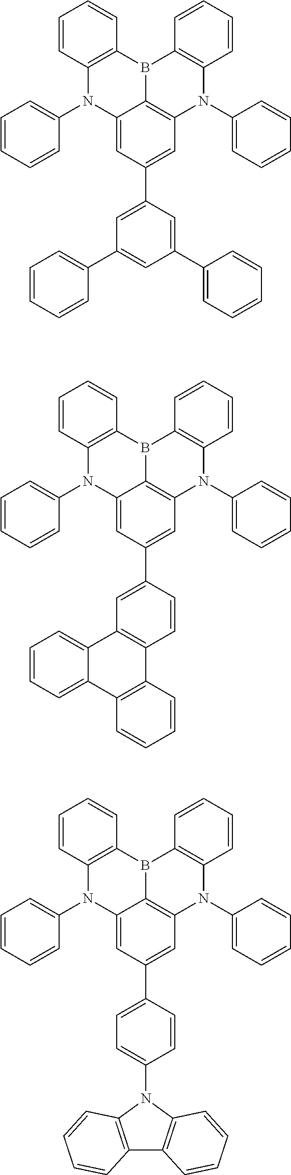 [SMILES: c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1]

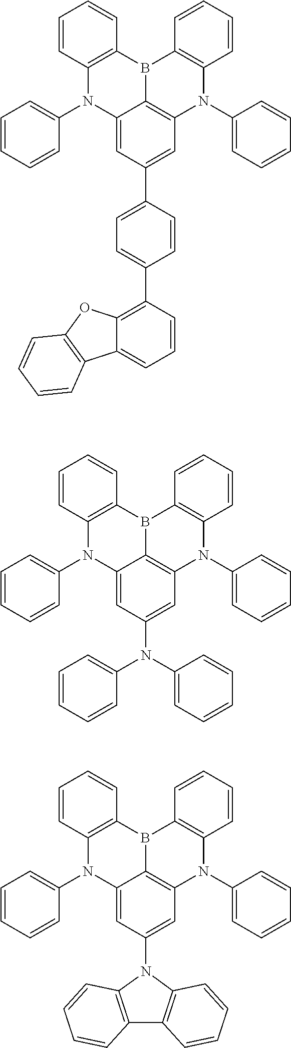 [SMILES: c1ccc(N2c3ccccc3B3c4ccccc4N(c4ccccc4)c4cc(-c5ccc(-c6cccc7c6oc6ccccc67)cc5)cc2c43)cc1 ; c1ccc(N(c2ccccc2)c2cc3c4c(c2)N(c2ccccc2)c2ccccc2B4c2ccccc2N3c2ccccc2)cc1 ; c1ccc(N2c3ccccc3B3c4ccccc4N(c4ccccc4)c4cc(-n5c6ccccc6c6ccccc65)cc2c43)cc1]

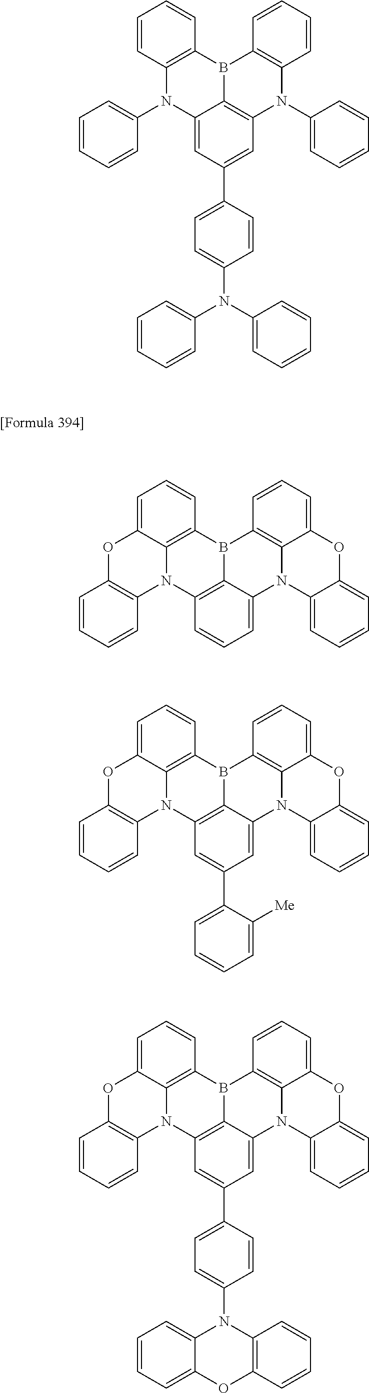 [SMILES: c1ccc(N(c2ccccc2)c2ccc(-c3cc4c5c(c3)N(c3ccccc3)c3ccccc3B5c3ccccc3N4c3ccccc3)cc2)cc1 ; c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1]

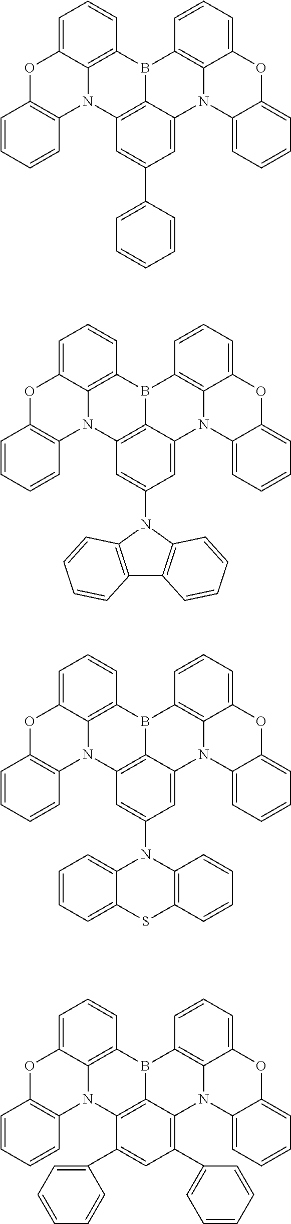 [SMILES: c1ccc(-c2cc3c4c(c2)N2c5ccccc5Oc5cccc(c52)B4c2cccc4c2N3c2ccccc2O4)cc1 ; c1ccc2c(c1)Oc1cccc3c1N2c1cc(-n2c4ccccc4c4ccccc42)cc2c1B3c1cccc3c1N2c1ccccc1O3 ; c1ccc2c(c1)Oc1cccc3c1N2c1cc(N2c4ccccc4Sc4ccccc42)cc2c1B3c1cccc3c1N2c1ccccc1O3 ; c1ccc(-c2cc(-c3ccccc3)c3c4c2N2c5ccccc5Oc5cccc(c52)B4c2cccc4c2N3c2ccccc2O4)cc1]

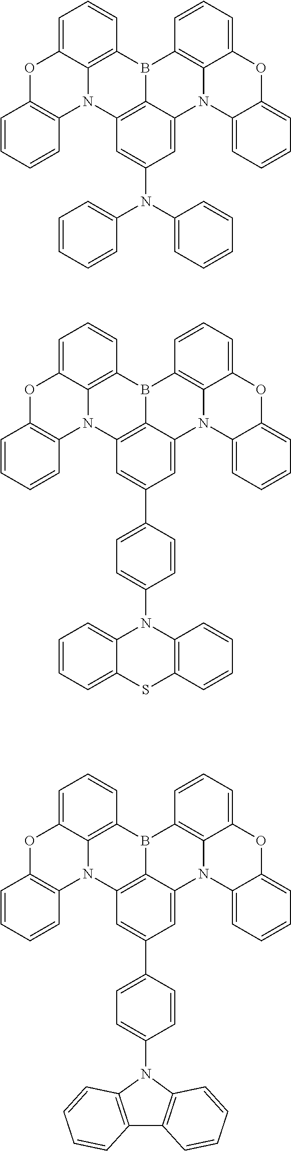 [SMILES: c1ccc(N(c2ccccc2)c2cc3c4c(c2)N2c5ccccc5Oc5cccc(c52)B4c2cccc4c2N3c2ccccc2O4)cc1 ; c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Oc1cccc3c1N2c1cc(-c2ccc(-n4c5ccccc5c5ccccc54)cc2)cc2c1B3c1cccc3c1N2c1ccccc1O3]

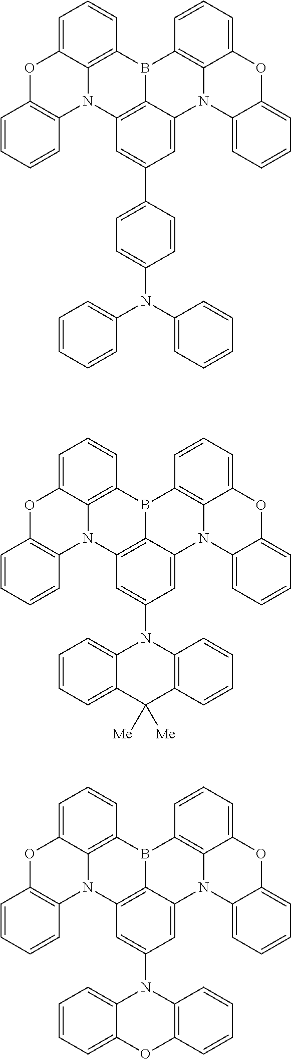 [SMILES: c1ccc(N(c2ccccc2)c2ccc(-c3cc4c5c(c3)N3c6ccccc6Oc6cccc(c63)B5c3cccc5c3N4c3ccccc3O5)cc2)cc1 ; CC1(C)c2ccccc2N(c2cc3c4c(c2)N2c5ccccc5Oc5cccc(c52)B4c2cccc4c2N3c2ccccc2O4)c2ccccc21 ; c1ccc2c(c1)Oc1ccccc1N2c1cc2c3c(c1)N1c4ccccc4Oc4cccc(c41)B3c1cccc3c1N2c1ccccc1O3]

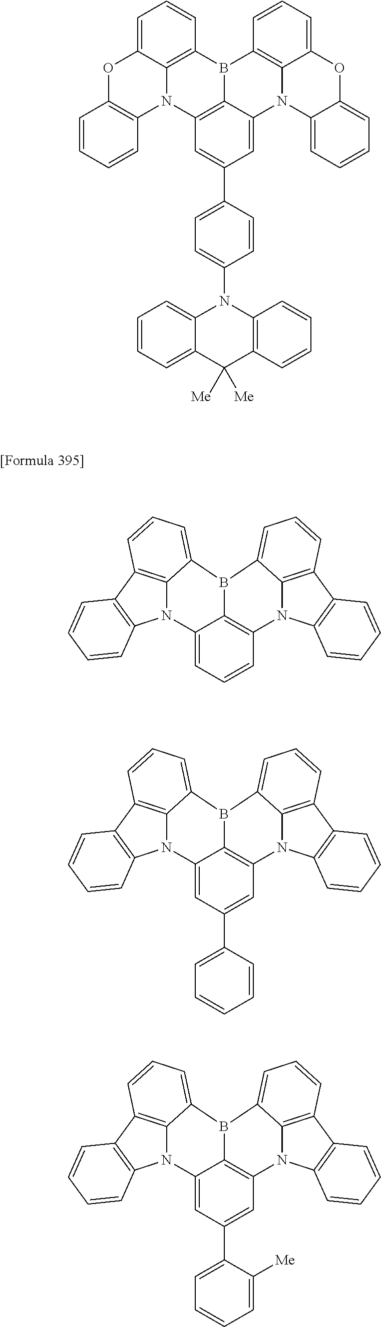 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

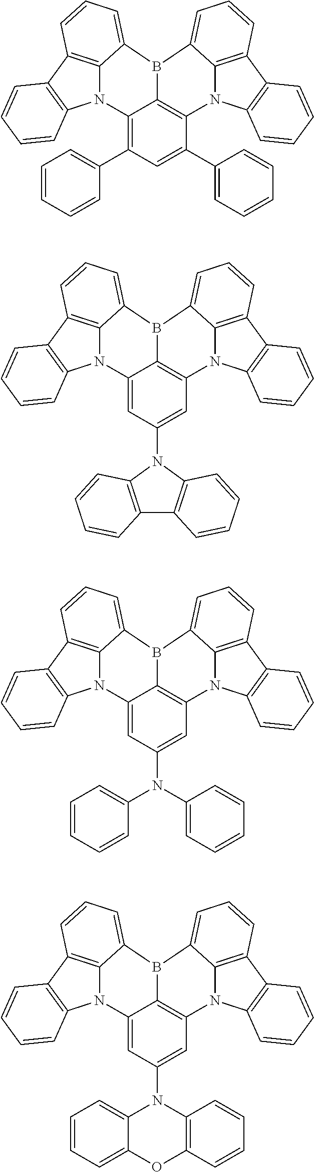 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc(N(c2ccccc2)c2cc3c4c(c2)-n2c5ccccc5c5cccc(c52)B4c2cccc4c5ccccc5n-3c24)cc1 ; c1ccc2c(c1)Oc1ccccc1N2c1cc2c3c(c1)-n1c4ccccc4c4cccc(c41)B3c1cccc3c4ccccc4n-2c13]

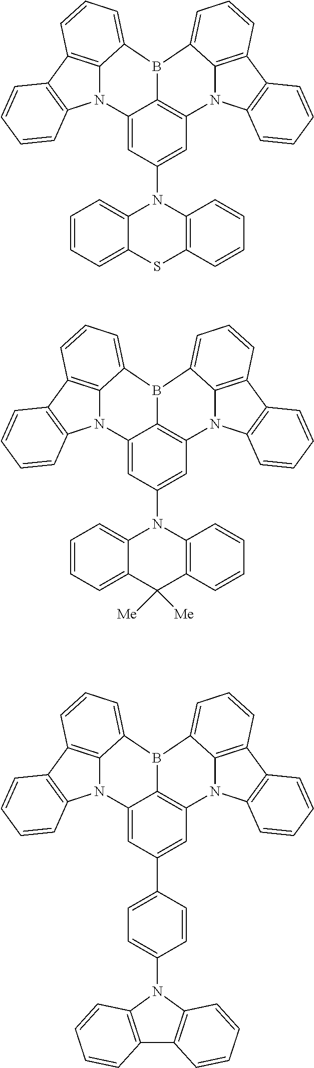 [SMILES: c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1]

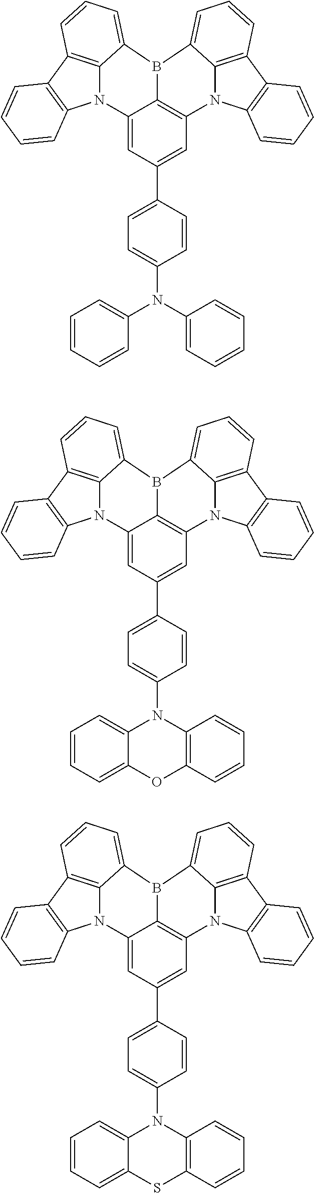

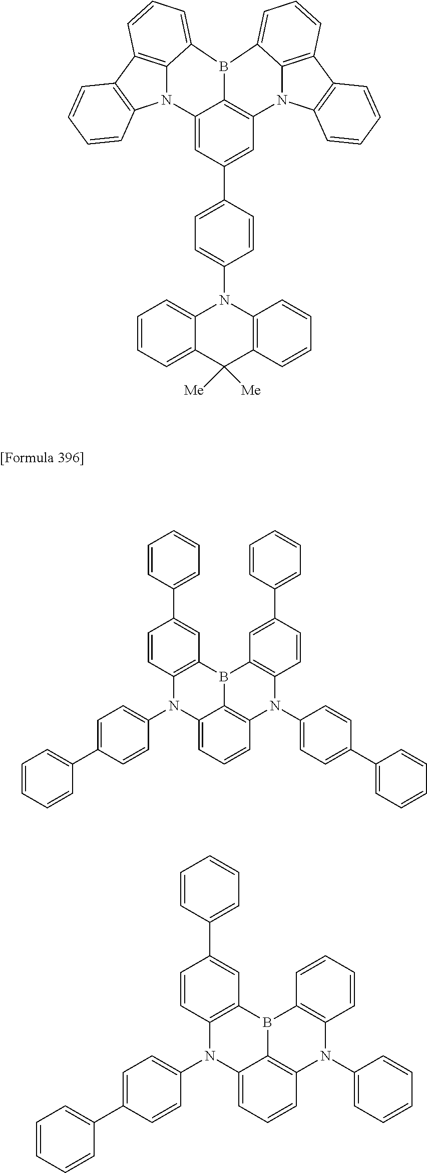 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1]

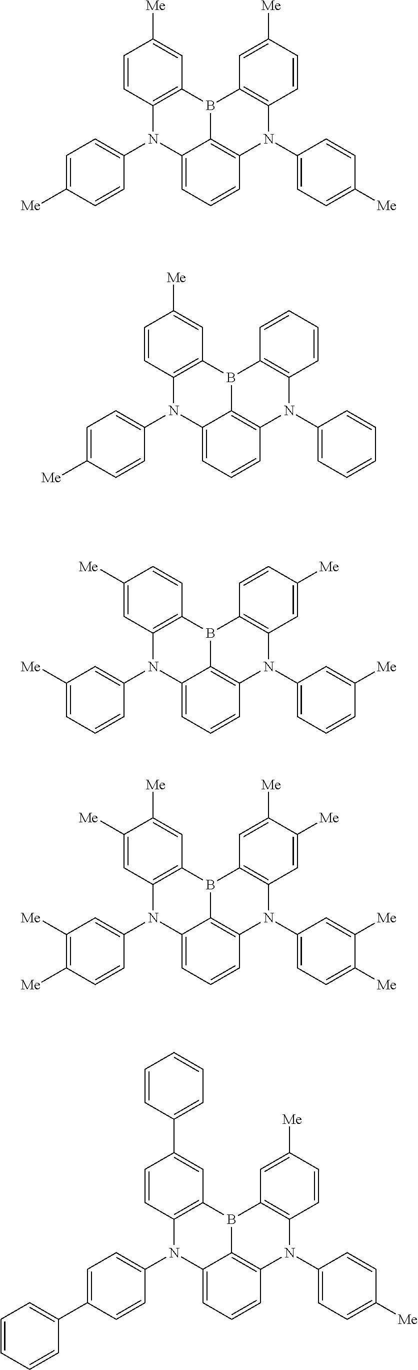 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1]

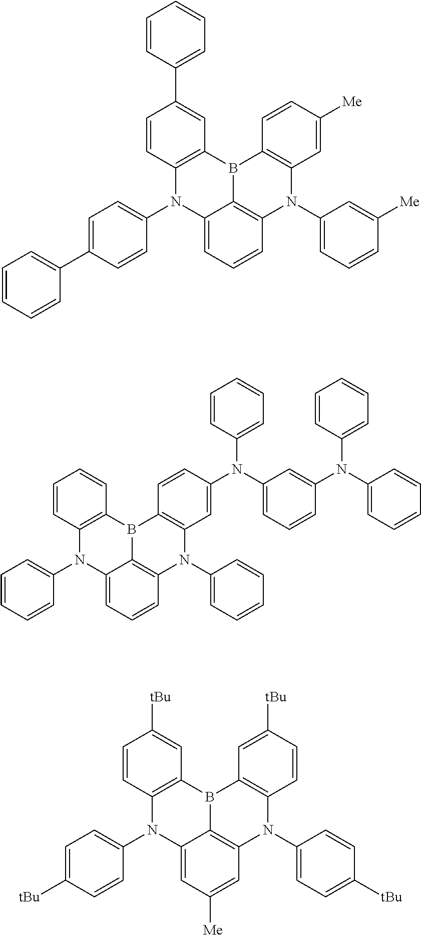 [SMILES: Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

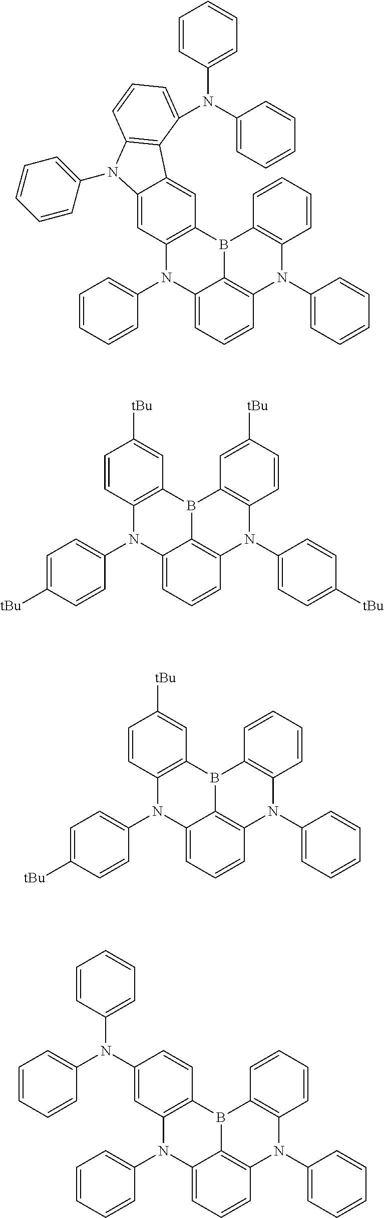 [SMILES: c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1]

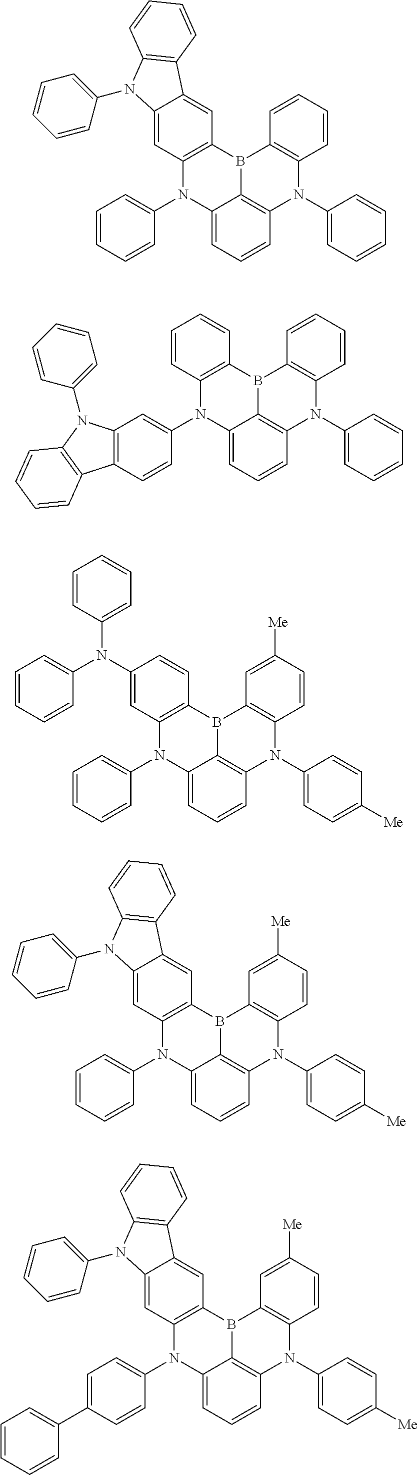 [SMILES: c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

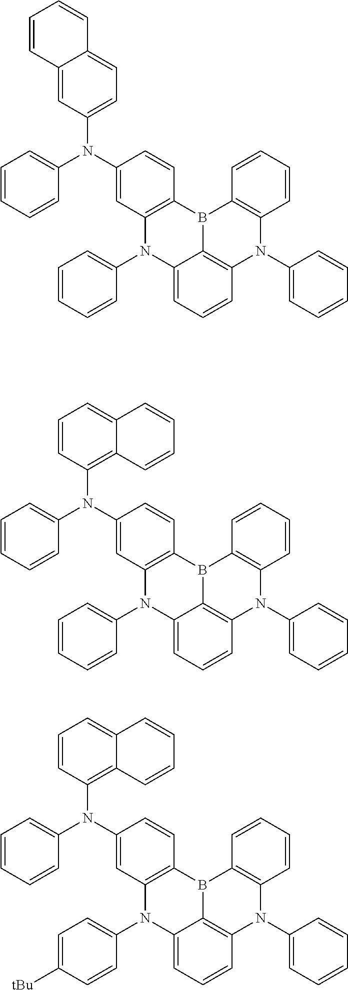 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1]

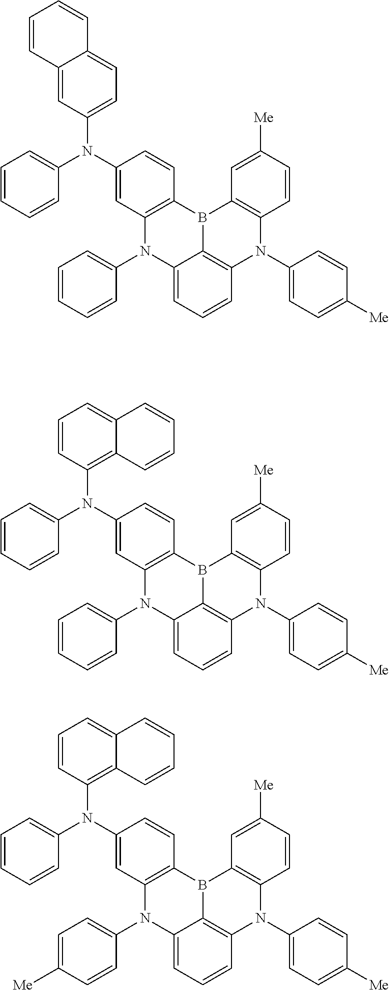 [SMILES: Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1]

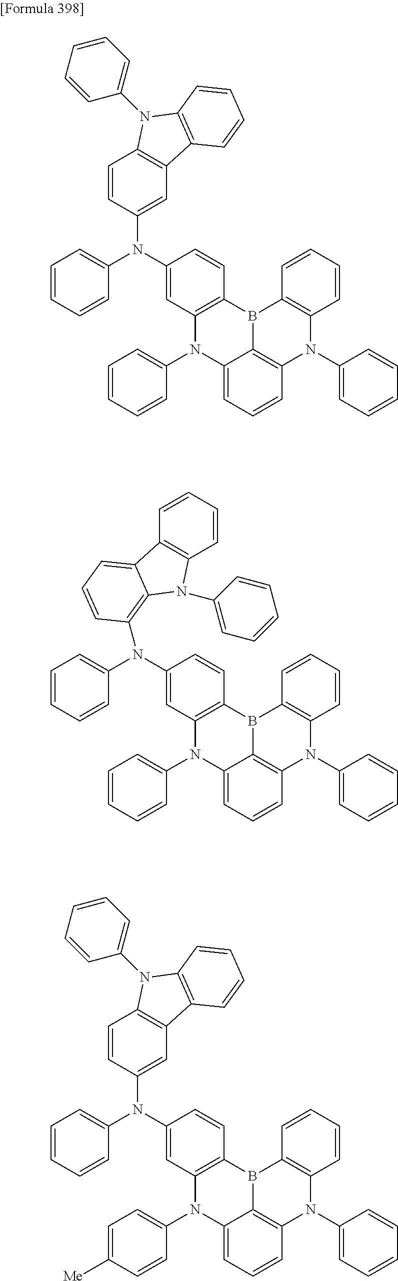 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

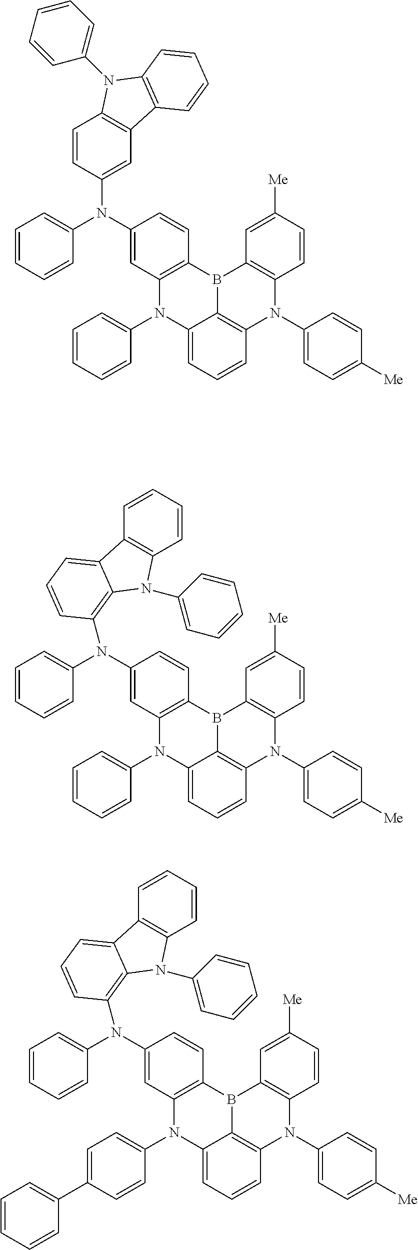 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

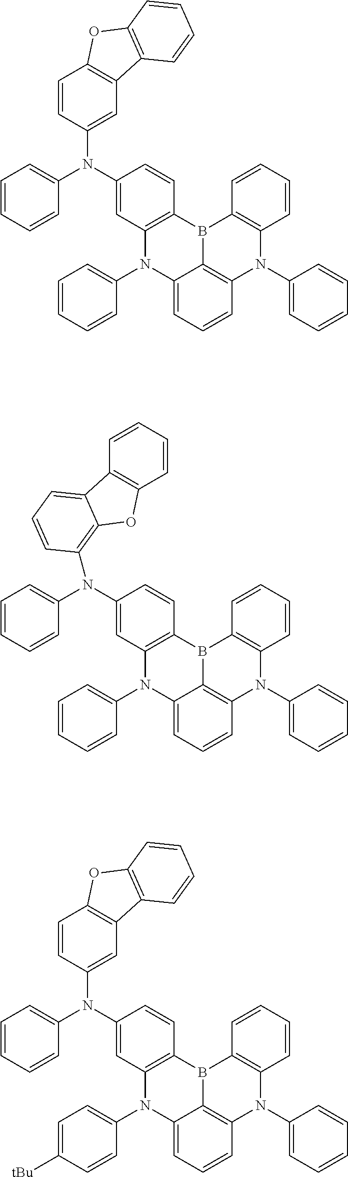 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

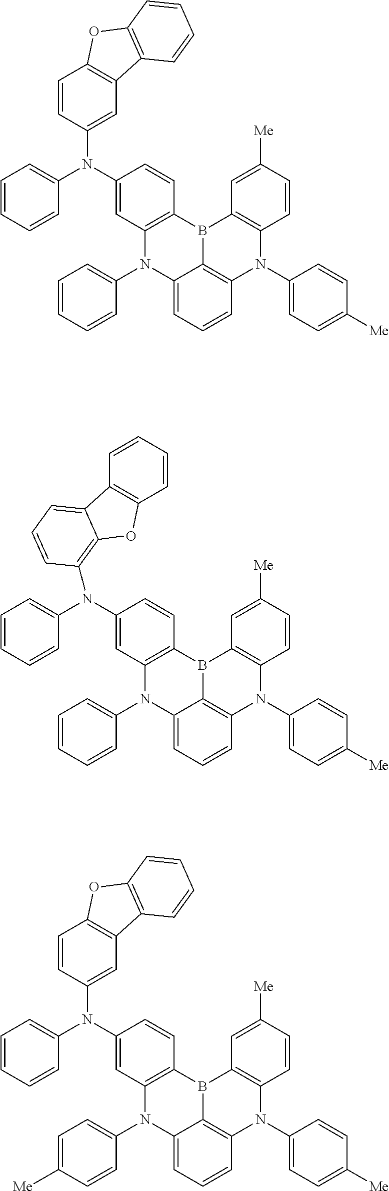 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

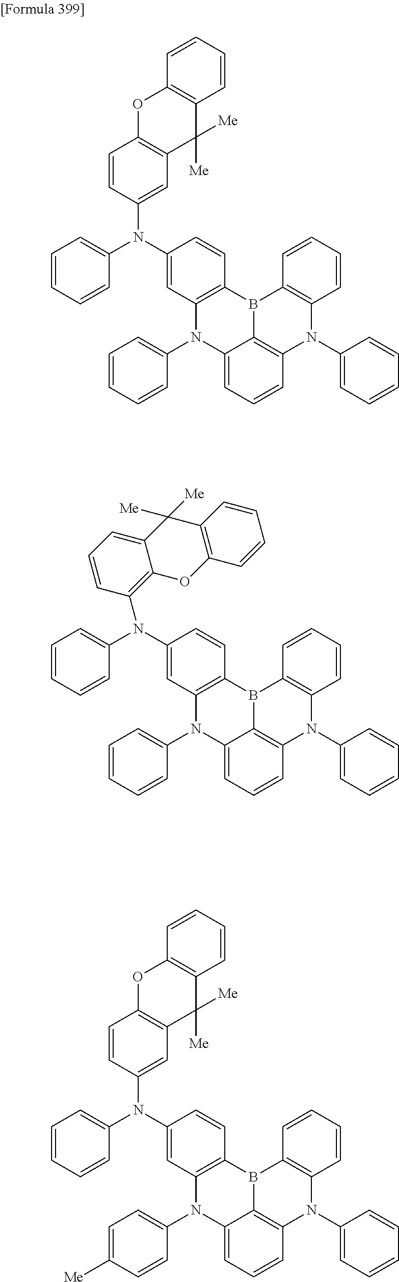 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

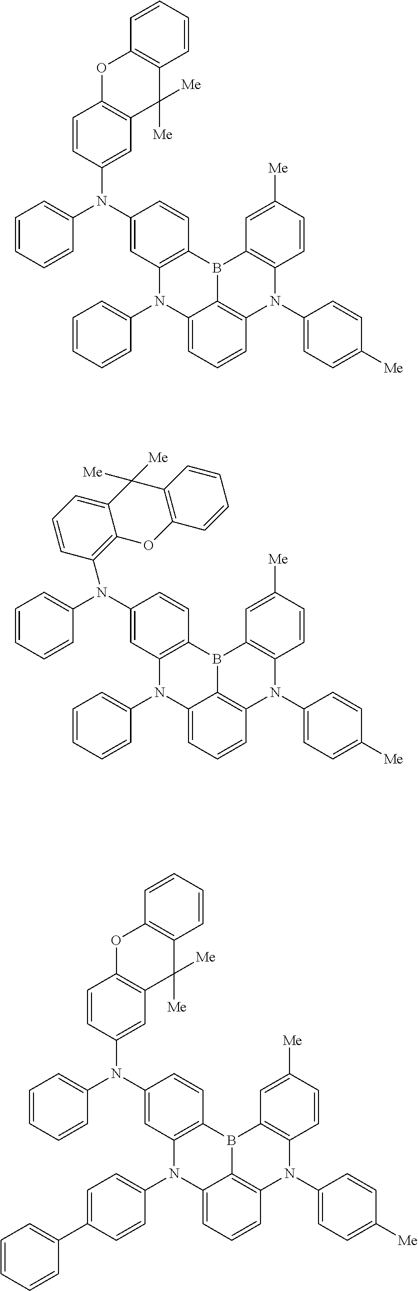 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

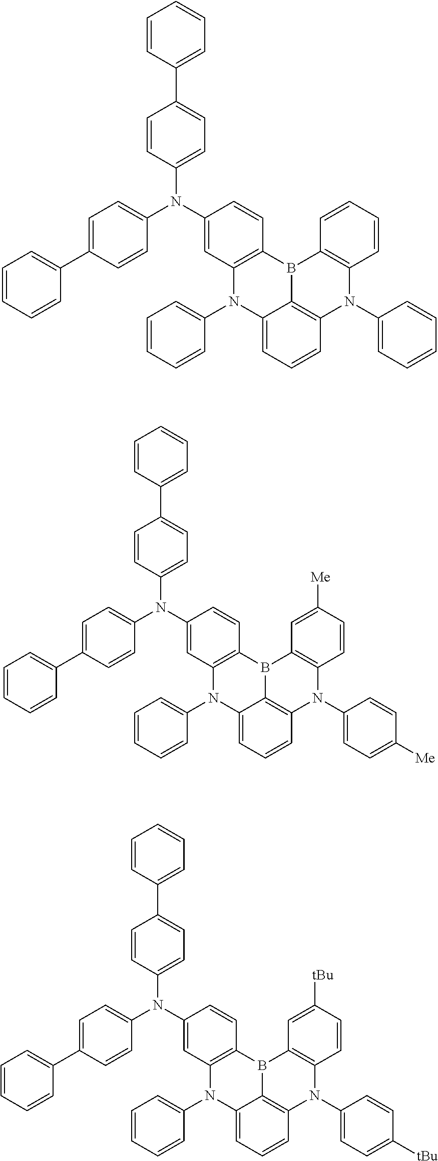 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

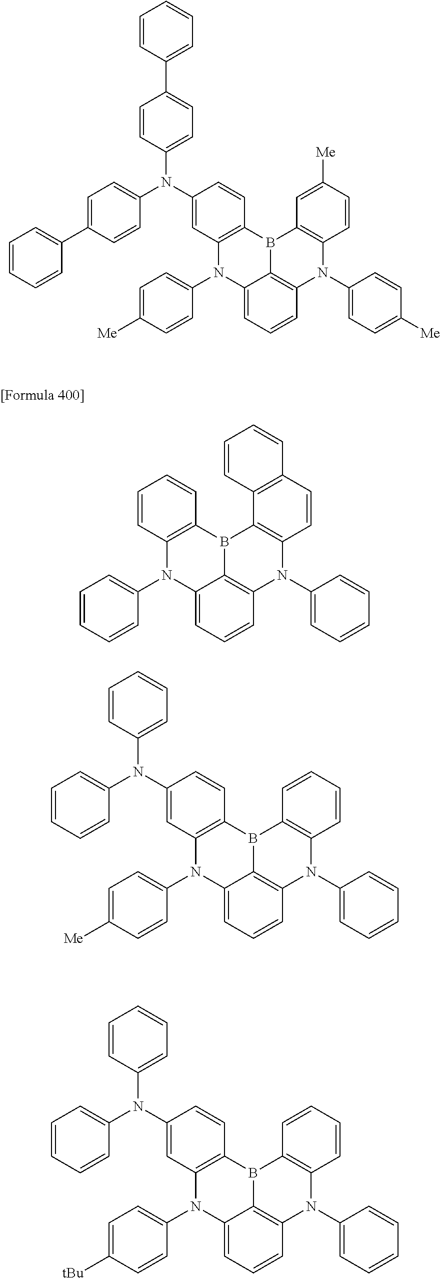

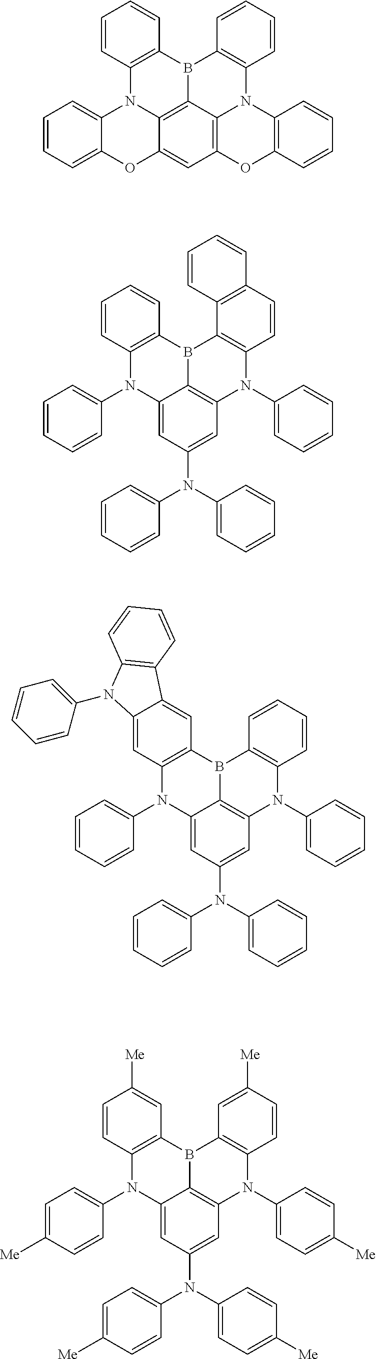 [SMILES: c1ccc2c(c1)B1c3ccccc3-n3c4ccccc4oc4cc5oc6ccccc6n-2c5c1c43 ; c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1]

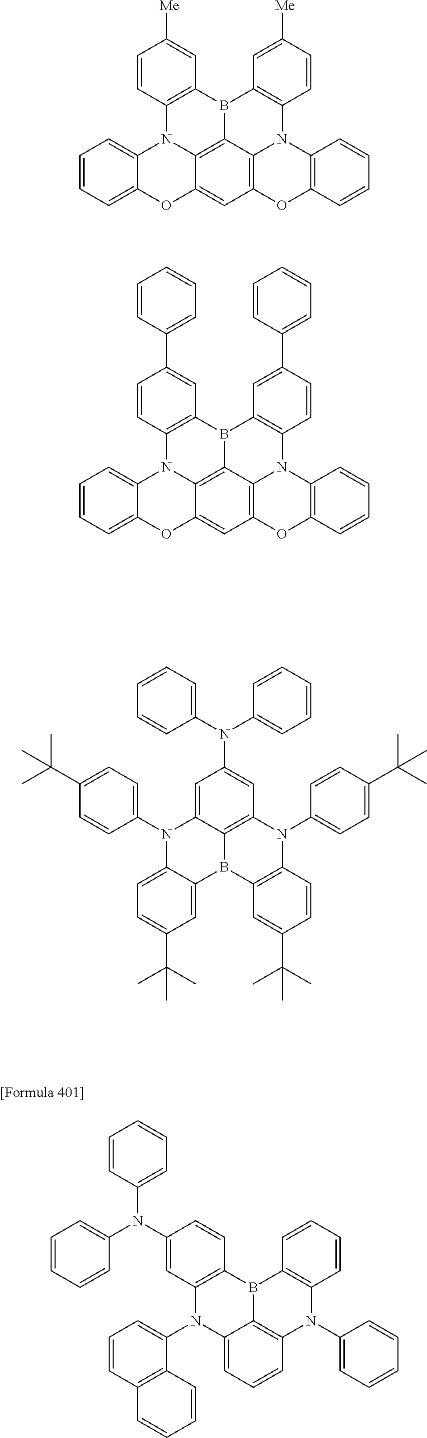 [SMILES: Cc1ccc2c(c1)B1c3cc(C)ccc3-n3c4ccccc4oc4cc5oc6ccccc6n-2c5c1c43 ; c1ccc(-c2ccc3c(c2)B2c4cc(-c5ccccc5)ccc4-n4c5ccccc5oc5cc6oc7ccccc7n-3c6c2c54)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; c1ccc(N(c2ccccc2)c2ccc3c(c2)N(c2cccc4ccccc24)c2cccc4c2B3c2ccccc2N4c2ccccc2)cc1]

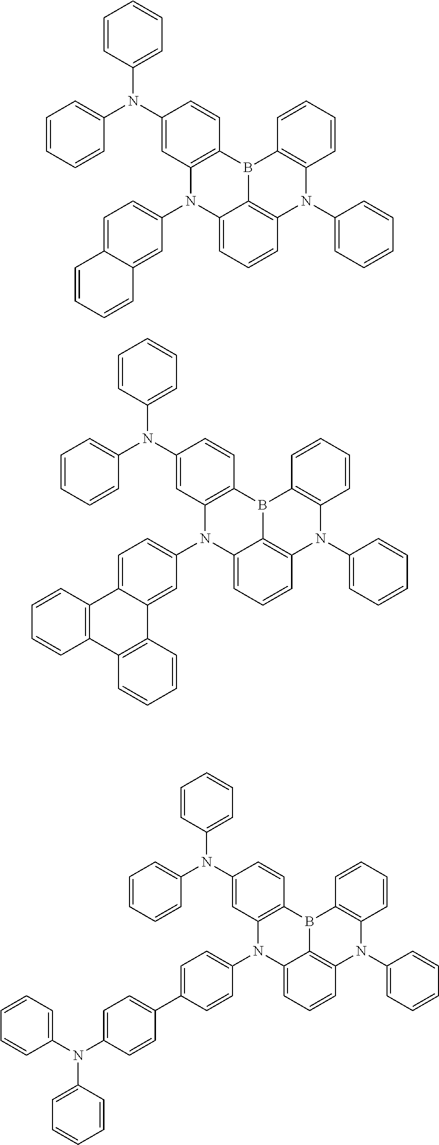

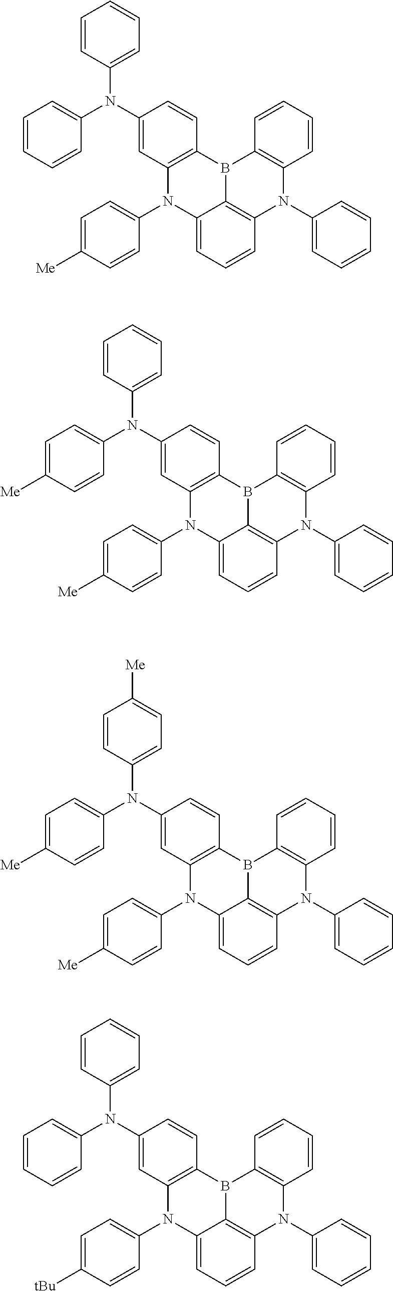 [SMILES: Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

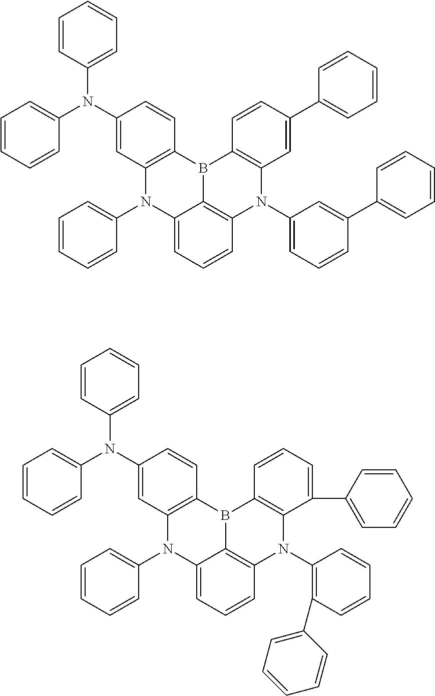 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

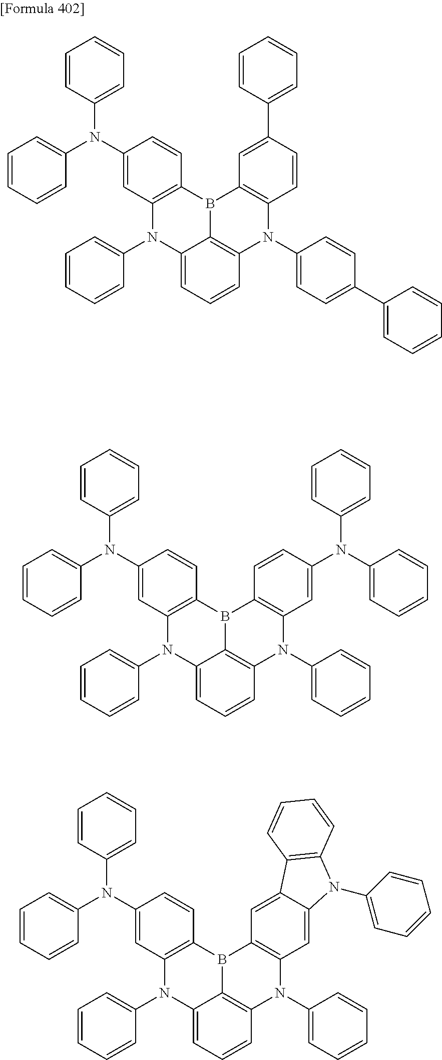 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1]

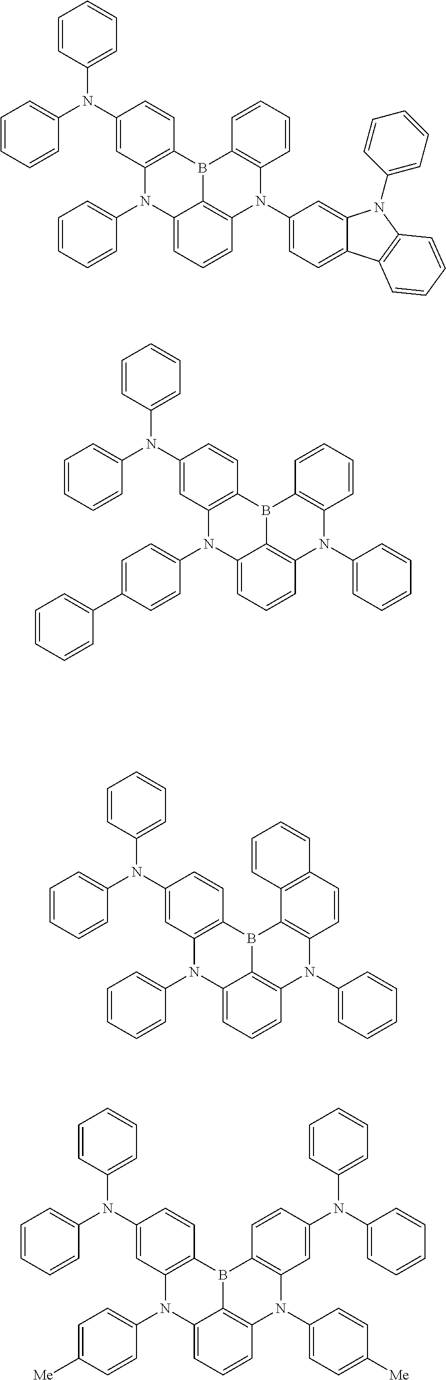 [SMILES: c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2c(ccc3ccccc23)N4c2ccccc2)cc1 ; Cc1ccc(N2c3cc(N(c4ccccc4)c4ccccc4)ccc3B3c4ccc(N(c5ccccc5)c5ccccc5)cc4N(c4ccc(C)cc4)c4cccc2c43)cc1]

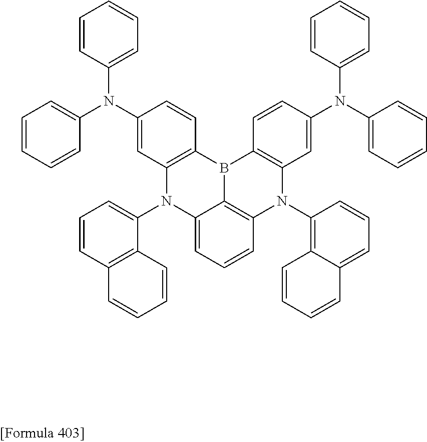 [SMILES: c1ccc(N(c2ccccc2)c2ccc3c(c2)N(c2cccc4ccccc24)c2cccc4c2B3c2ccc(N(c3ccccc3)c3ccccc3)cc2N4c2cccc3ccccc23)cc1]

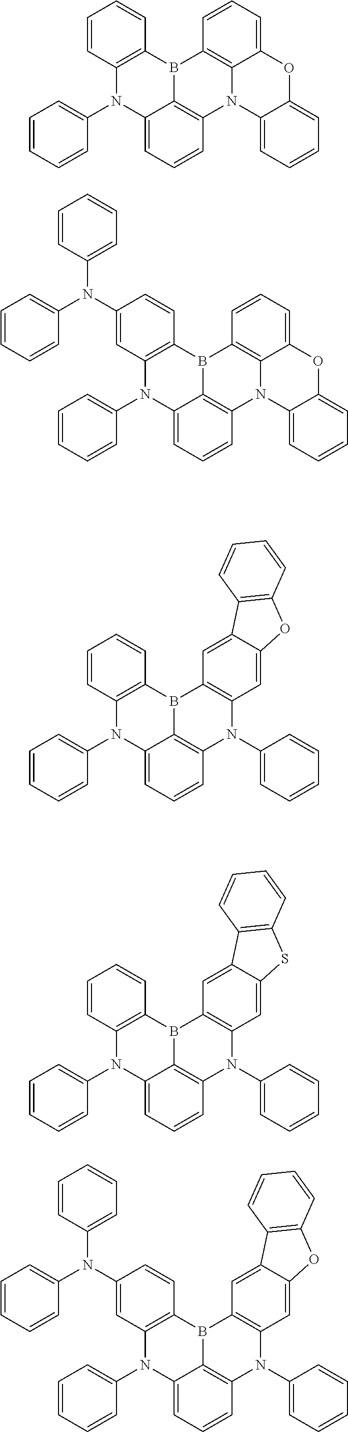

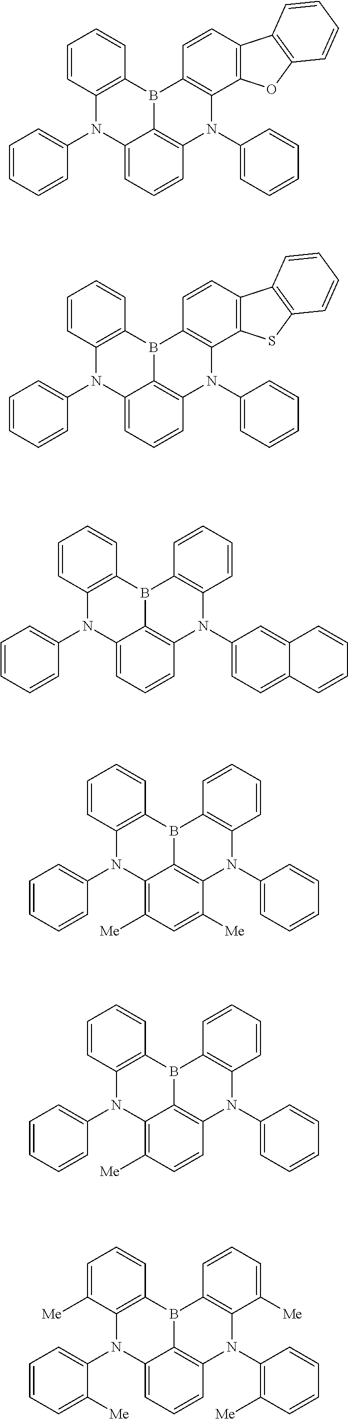 [SMILES: c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C]

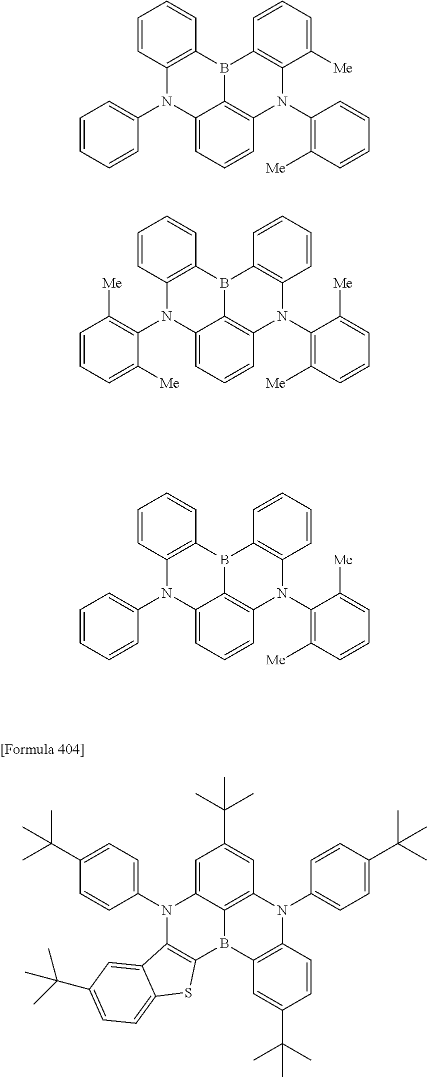 [SMILES: Cc1ccccc1N1c2cccc3c2B(c2ccccc2N3c2ccccc2)c2cccc(C)c21 ; Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32 ; CC(C)(C)c1ccc(N2c3ccc(C(C)(C)C)cc3B3c4sc5ccc(C(C)(C)C)cc5c4N(c4ccc(C(C)(C)C)cc4)c4cc(C(C)(C)C)cc2c43)cc1]

### Compound Represented by Formula (3)

The compound represented by the formula (3) will be described below.

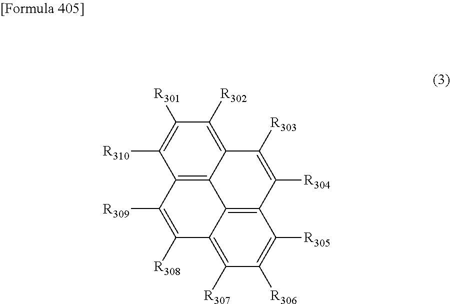

In the formula (3):
at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded;
at least one of R₃₀₁ to R₃₁₀ is a monovalent group represented by a formula (31) below; and
R₃₀₁ to R₃₁₀ forming neither the monocyclic ring nor the substituted or unsubstituted fused ring and not being a monovalent group represented by a formula (31) below are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

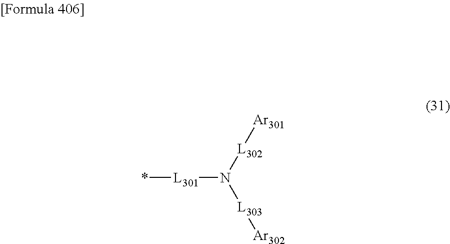

In the formula (31):
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;
L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position to a pyrene ring in the formula (3).

In the luminescent compound, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms; preferably, a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of Roos are present, the plurality of Roos are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In the formula (3), two of R₃₀₁ to R₃₁₀ are each preferably a group represented by the formula (31).

In an exemplary embodiment, the compound represented by the formula (3) is a compound represented by a formula (33) below.

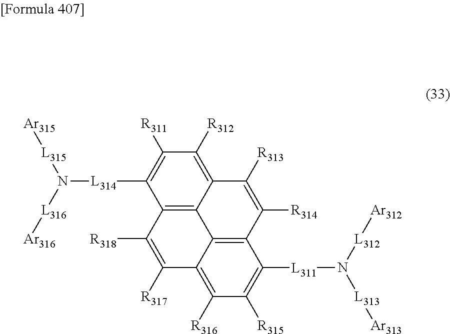

In the formula (33):
R₃₁₁ to R₃₁₈ each independently represent the same as R₃₀₁ to R₃₁₀ in the formula (3) that are not a monovalent group represented by the formula (31);
L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms; and
Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are each preferably a single bond.

In an exemplary embodiment, the compound represented by the formula (3) is represented by a formula (34) or a formula (35) below.

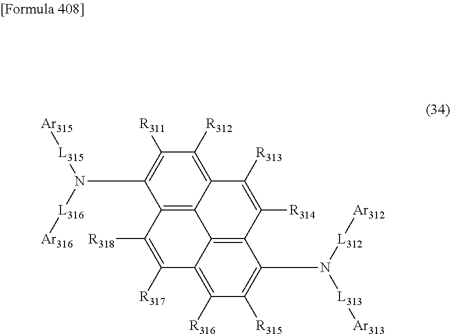

In the formula (34):
R₃₁₁ to R₃₁₈ each independently represent the same as R₃₀₁ to R₃₁₀ in the formula (3) that are not a monovalent group represented by the formula (31);
L₃₁₂, L₃₁₃, L₃₁₅ and L₃₁₆ each independently represent the same as L₃₁₂, L₃₁₃, L₃₁₅ and L₃₁₆ in the formula (33); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

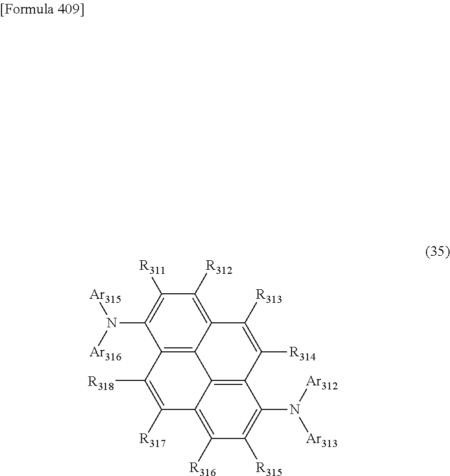

In the formula (35):
R₃₁₁ to R₃₁₈ each independently represent the same as R₃₀₁ to R₃₁₀ in the formula (3) that are not a monovalent group represented by the formula (31);
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

In the formula (31), at least one of Ar₃₀₁ or Ar₃₀₂ is preferably a group represented by a formula (36) below.

In the formulae (33) to (35), at least one of Ar₃₁₂ or Ar₃₁₃ is preferably a group represented by the formula (36).

In the formulae (33) to (35), at least one of Ar₃₁₅ or Ar₃₁₆ is preferably a group represented by the formula (36).

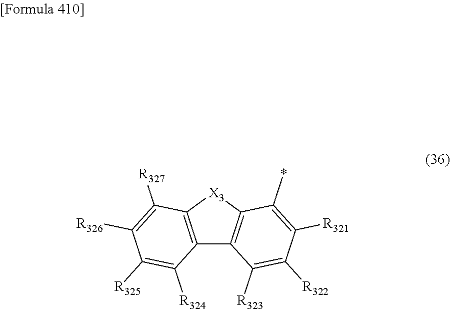

In the formula (36):
X₃ represents an oxygen atom or a sulfur atom;
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
R₃₂₁ to R₃₂₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position to L₃₀₂, L₃₀₃, L₃₁₂, L₃₁₃, L₃₁₅ Or L₃₁₆.
X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), preferably, Ar₃₀₁ is a group represented by the formula (36) and Ar₃₀₂ is a substituted or unsubstituted aryl group having 6 to 50 ring carbon atoms.

In the formulae (33) to (35), preferably, Ar₃₁₂ is a group represented by the formula (36) and Ar₃₁₃ is a substituted or unsubstituted aryl group having 6 to 50 ring carbon atoms.

In the formulae (33) to (35), preferably, Ar₃₁₅ is a group represented by the formula (36) and Ar₃₁₆ is a substituted or unsubstituted aryl group having 6 to 50 ring carbon atoms.

In an exemplary embodiment, the compound represented by the formula (3) is represented by a formula (37) below.

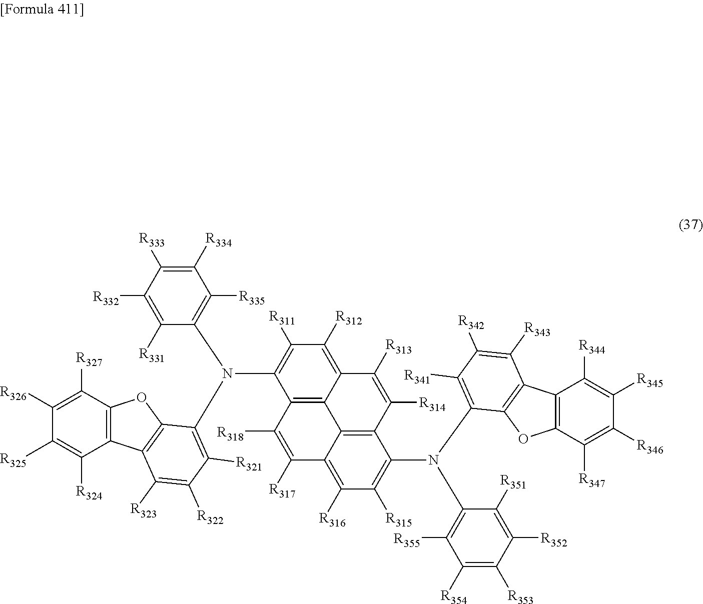

In the formula (37):
R₃₁₁ to R₃₁₈ each independently represent the same as R₃₀₁ to R₃₁₀ in the formula (3) that are not a monovalent group represented by the formula (31);
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;
R₃₂₁ to R₃₂₇ and R₃₄₁ to R₃₄₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₃₁ to R₃₃₅ and R₃₅₁ to R₃₅₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (3)

Specific examples of the compound represented by the formula (3) include compounds as below.

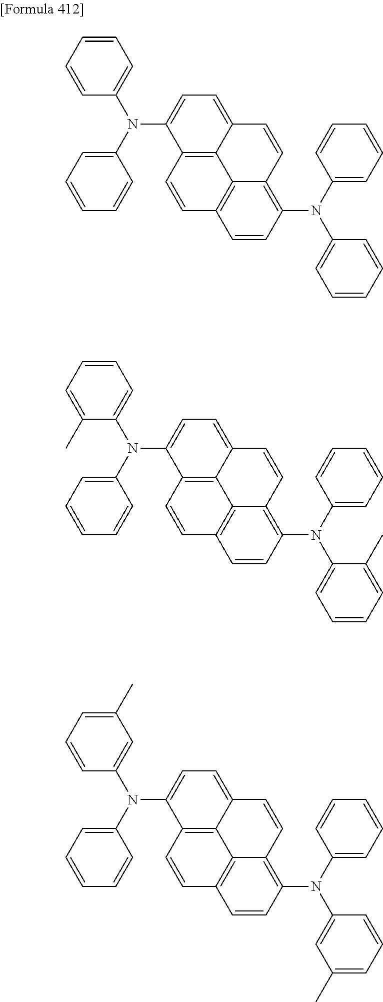 [SMILES: c1ccc(N(c2ccccc2)c2ccc3ccc4c(N(c5ccccc5)c5ccccc5)ccc5ccc2c3c54)cc1 ; Cc1ccccc1N(c1ccccc1)c1ccc2ccc3c(N(c4ccccc4)c4ccccc4C)ccc4ccc1c2c43 ; Cc1cccc(N(c2ccccc2)c2ccc3ccc4c(N(c5ccccc5)c5cccc(C)c5)ccc5ccc2c3c54)c1]

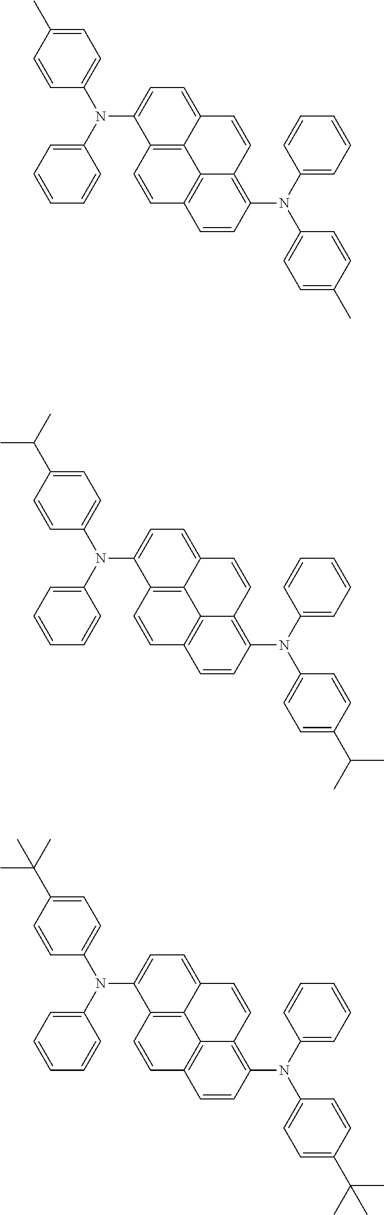

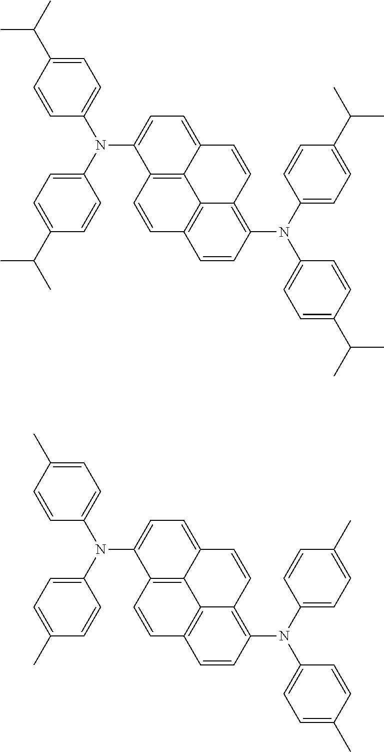 [SMILES: CC(C)c1ccc(N(c2ccc(C(C)C)cc2)c2ccc3ccc4c(N(c5ccc(C(C)C)cc5)c5ccc(C(C)C)cc5)ccc5ccc2c3c54)cc1 ; Cc1ccc(N(c2ccc(C)cc2)c2ccc3ccc4c(N(c5ccc(C)cc5)c5ccc(C)cc5)ccc5ccc2c3c54)cc1]

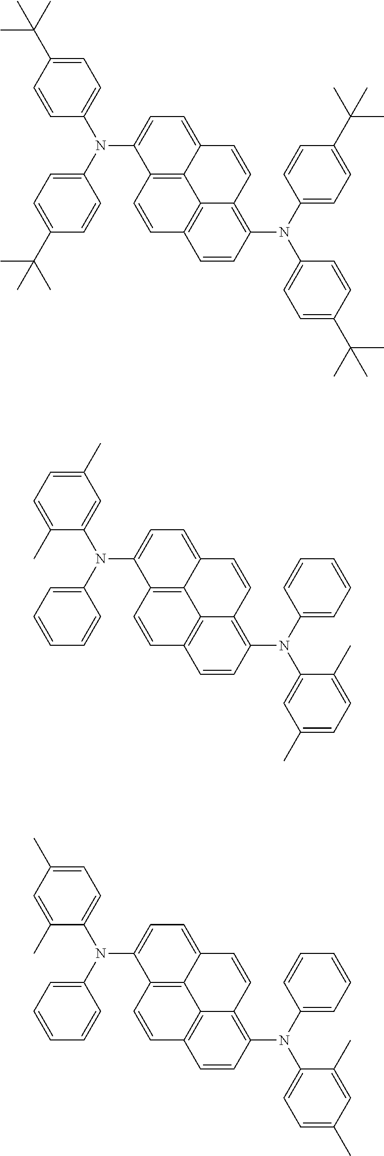 [SMILES: CC(C)(C)c1ccc(N(c2ccc(C(C)(C)C)cc2)c2ccc3ccc4c(N(c5ccc(C(C)(C)C)cc5)c5ccc(C(C)(C)C)cc5)ccc5ccc2c3c54)cc1 ; Cc1ccc(C)c(N(c2ccccc2)c2ccc3ccc4c(N(c5ccccc5)c5cc(C)ccc5C)ccc5ccc2c3c54)c1 ; Cc1ccc(N(c2ccccc2)c2ccc3ccc4c(N(c5ccccc5)c5ccc(C)cc5C)ccc5ccc2c3c54)c(C)c1]

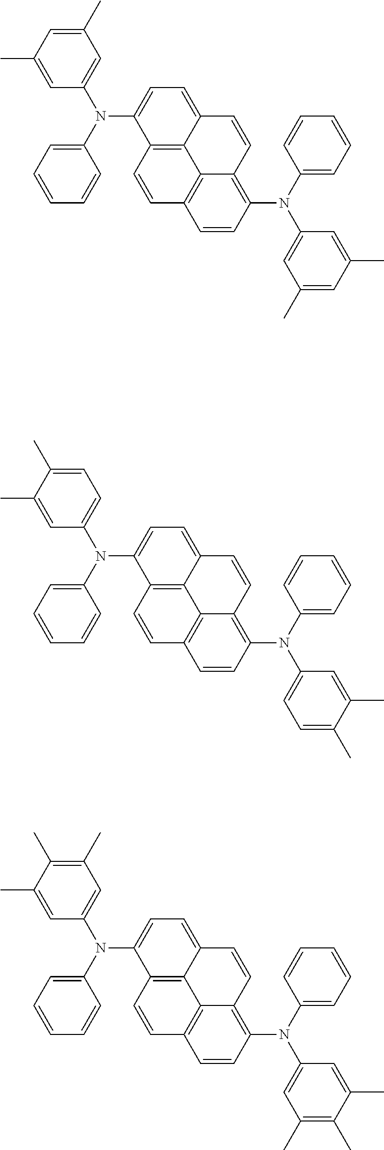

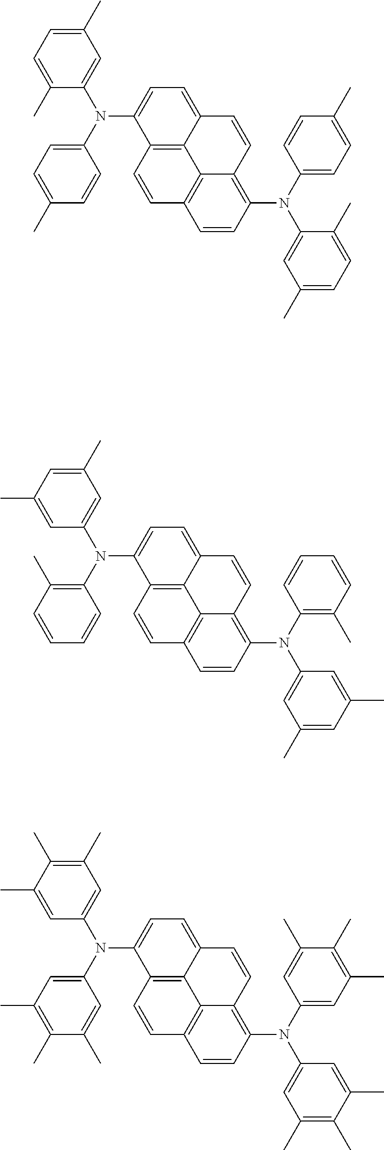

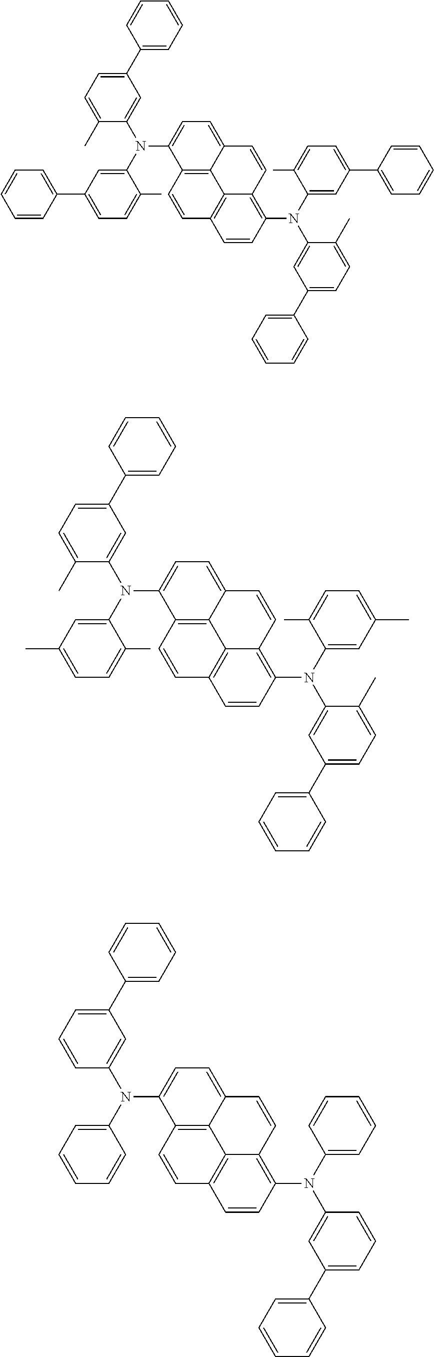

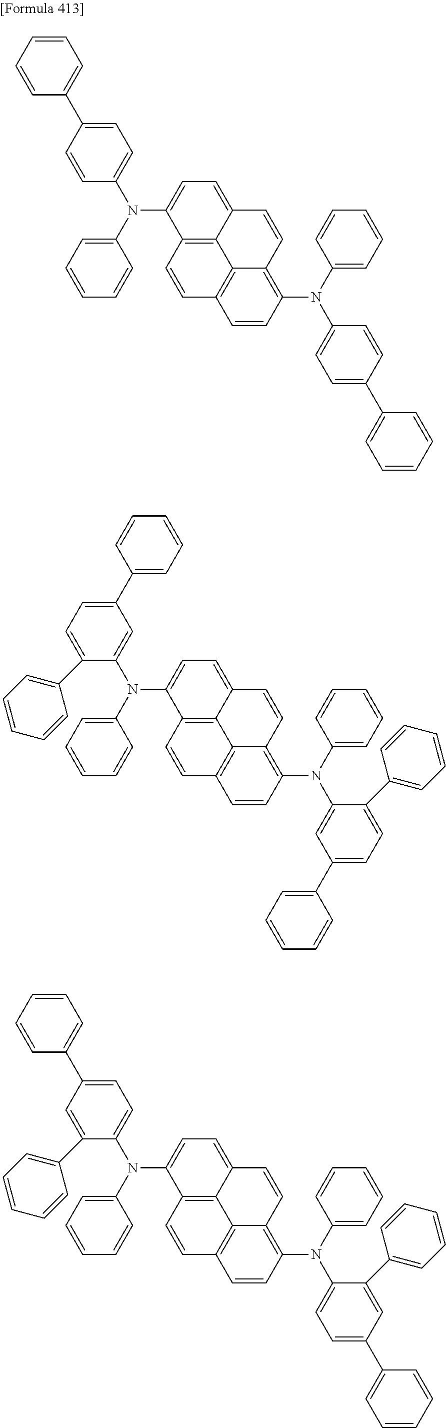 [SMILES: c1ccc(-c2ccc(N(c3ccccc3)c3ccc4ccc5c(N(c6ccccc6)c6ccc(-c7ccccc7)cc6)ccc6ccc3c4c65)cc2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1 ; c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1]

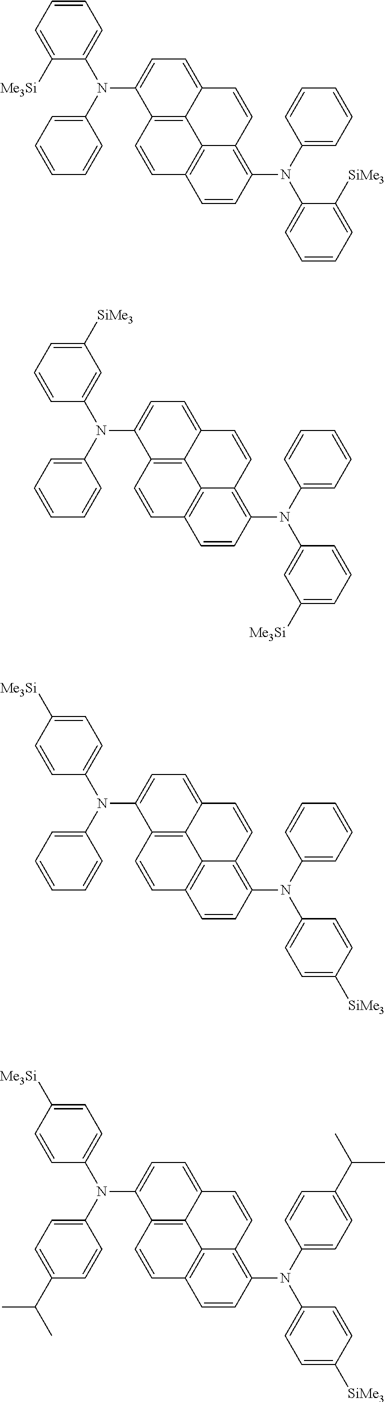 [SMILES: C[Si](C)(C)c1ccccc1N(c1ccccc1)c1ccc2ccc3c(N(c4ccccc4)c4ccccc4[Si](C)(C)C)ccc4ccc1c2c43 ; C[Si](C)(C)c1cccc(N(c2ccccc2)c2ccc3ccc4c(N(c5ccccc5)c5cccc([Si](C)(C)C)c5)ccc5ccc2c3c54)c1 ; C[Si](C)(C)c1ccc(N(c2ccccc2)c2ccc3ccc4c(N(c5ccccc5)c5ccc([Si](C)(C)C)cc5)ccc5ccc2c3c54)cc1 ; CC(C)c1ccc(N(c2ccc([Si](C)(C)C)cc2)c2ccc3ccc4c(N(c5ccc(C(C)C)cc5)c5ccc([Si](C)(C)C)cc5)ccc5ccc2c3c54)cc1]

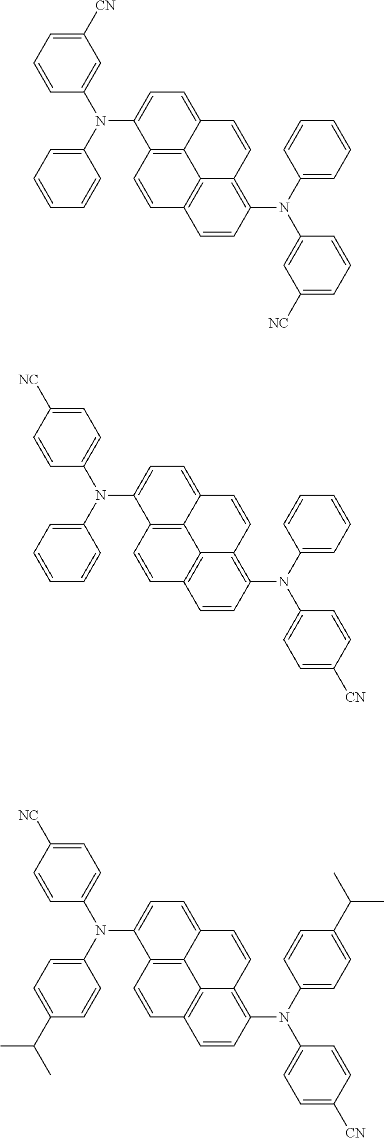 [SMILES: N#Cc1cccc(N(c2ccccc2)c2ccc3ccc4c(N(c5ccccc5)c5cccc(C#N)c5)ccc5ccc2c3c54)c1 ; N#Cc1ccc(N(c2ccccc2)c2ccc3ccc4c(N(c5ccccc5)c5ccc(C#N)cc5)ccc5ccc2c3c54)cc1 ; CC(C)c1ccc(N(c2ccc(C#N)cc2)c2ccc3ccc4c(N(c5ccc(C#N)cc5)c5ccc(C(C)C)cc5)ccc5ccc2c3c54)cc1]

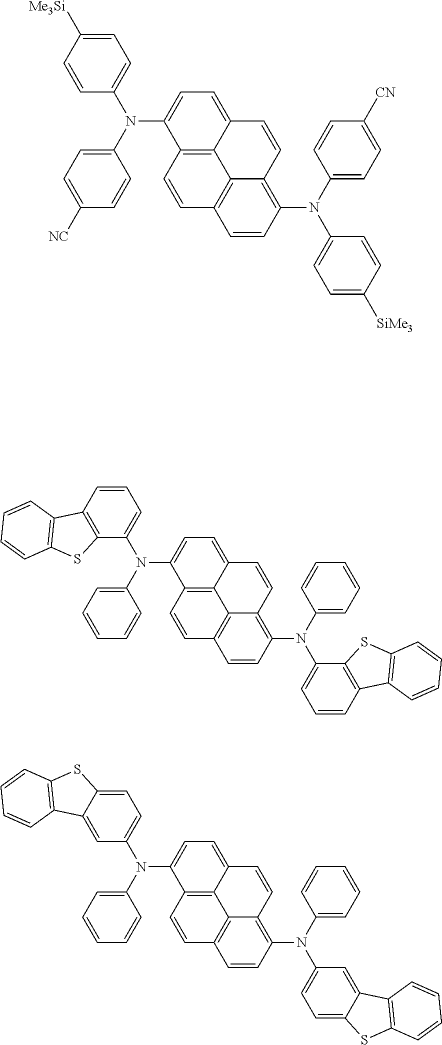 [SMILES: C[Si](C)(C)c1ccc(N(c2ccc(C#N)cc2)c2ccc3ccc4c(N(c5ccc(C#N)cc5)c5ccc([Si](C)(C)C)cc5)ccc5ccc2c3c54)cc1 ; c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1]

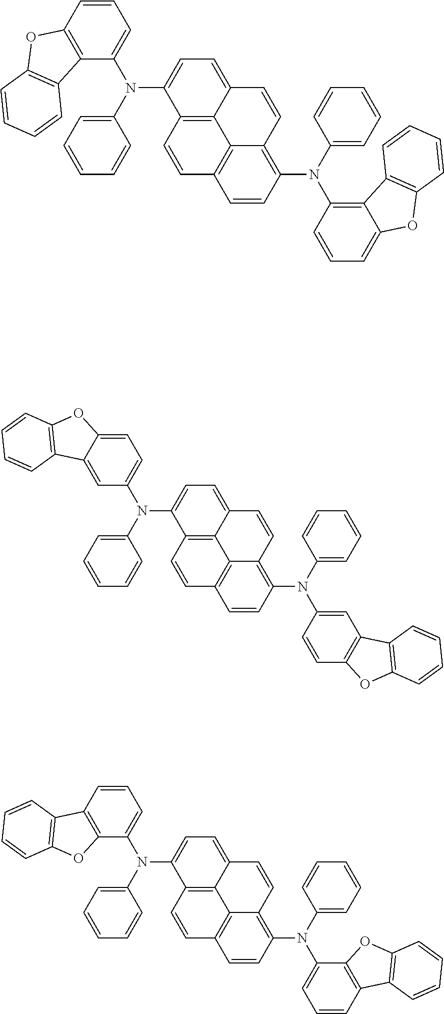 [SMILES: c1ccc(N(c2ccc3ccc4c(N(c5ccccc5)c5cccc6oc7ccccc7c56)ccc5ccc2c3c54)c2cccc3oc4ccccc4c23)cc1 ; c1ccc(N(c2ccc3oc4ccccc4c3c2)c2ccc3ccc4c(N(c5ccccc5)c5ccc6oc7ccccc7c6c5)ccc5ccc2c3c54)cc1 ; c1ccc(N(c2ccc3ccc4c(N(c5ccccc5)c5cccc6c5oc5ccccc56)ccc5ccc2c3c54)c2cccc3c2oc2ccccc23)cc1]

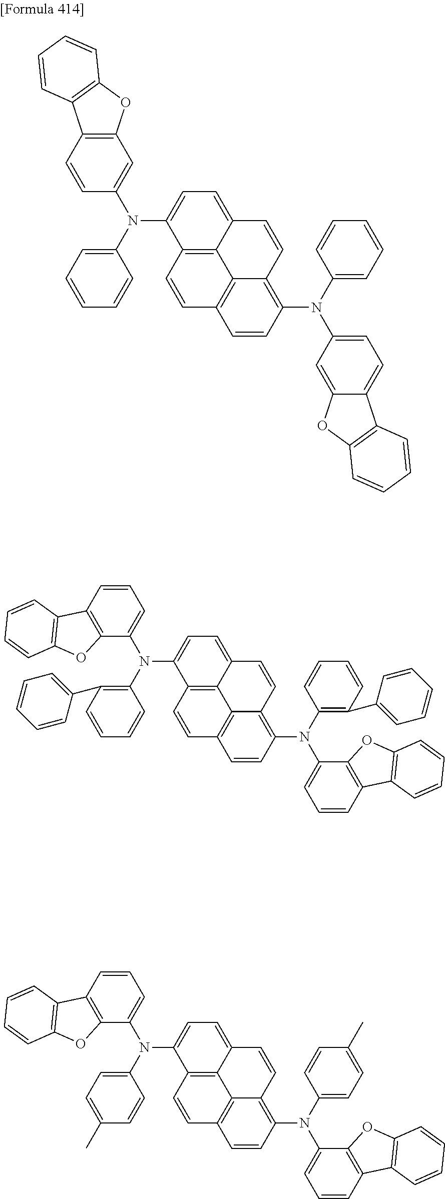 [SMILES: c1ccc(N(c2ccc3c(c2)oc2ccccc23)c2ccc3ccc4c(N(c5ccccc5)c5ccc6c(c5)oc5ccccc56)ccc5ccc2c3c54)cc1 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; Cc1ccc(N(c2ccc3ccc4c(N(c5ccc(C)cc5)c5cccc6c5oc5ccccc56)ccc5ccc2c3c54)c2cccc3c2oc2ccccc23)cc1]

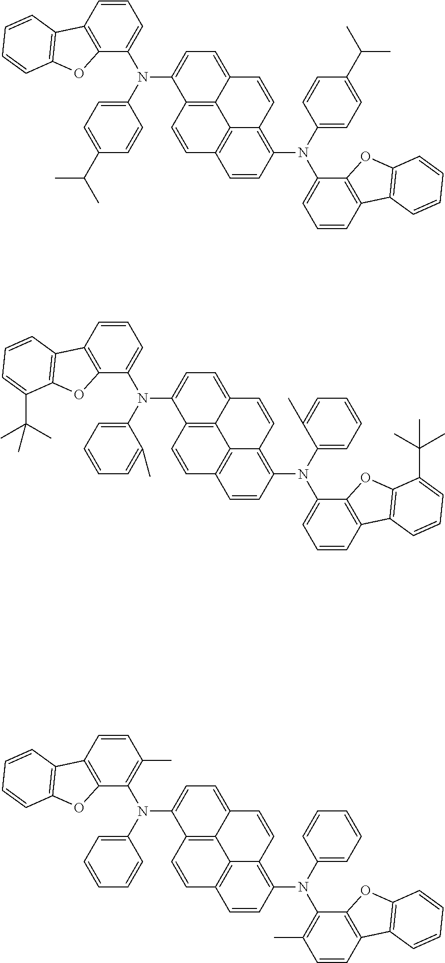 [SMILES: CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12 ; Cc1ccc2c(oc3ccccc32)c1N(c1ccccc1)c1ccc2ccc3c(N(c4ccccc4)c4c(C)ccc5c4oc4ccccc45)ccc4ccc1c2c43]

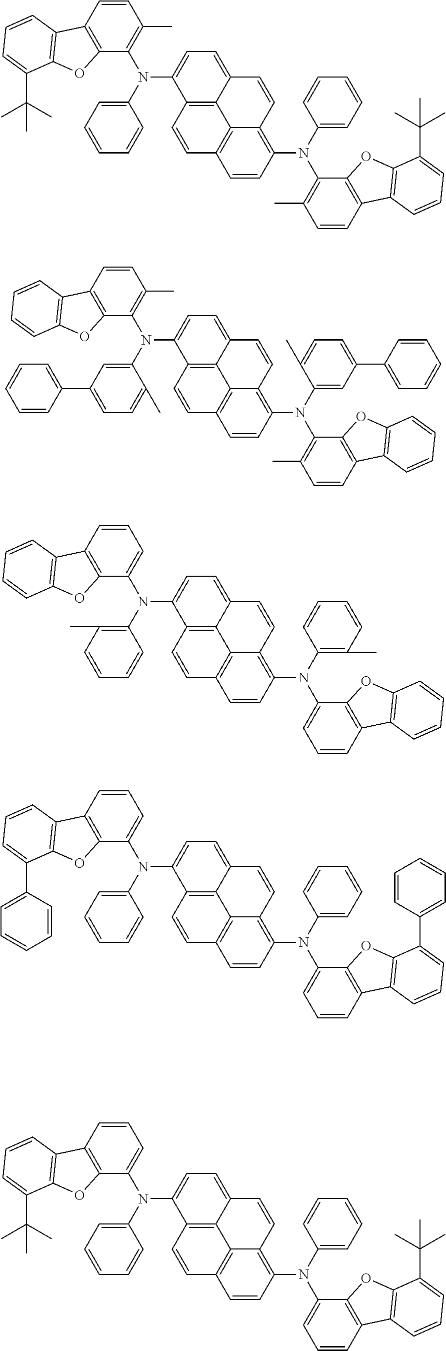

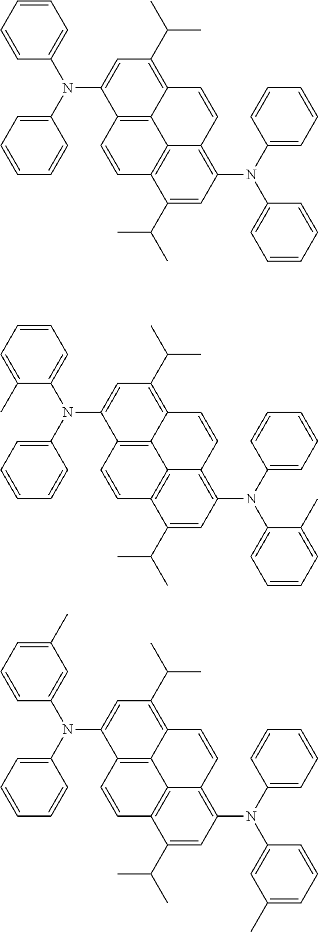 [SMILES: CC(C)c1cc(N(c2ccccc2)c2ccccc2)c2ccc3c(C(C)C)cc(N(c4ccccc4)c4ccccc4)c4ccc1c2c34 ; Cc1ccccc1N(c1ccccc1)c1cc(C(C)C)c2ccc3c(N(c4ccccc4)c4ccccc4C)cc(C(C)C)c4ccc1c2c43 ; Cc1cccc(N(c2ccccc2)c2cc(C(C)C)c3ccc4c(N(c5ccccc5)c5cccc(C)c5)cc(C(C)C)c5ccc2c3c54)c1]

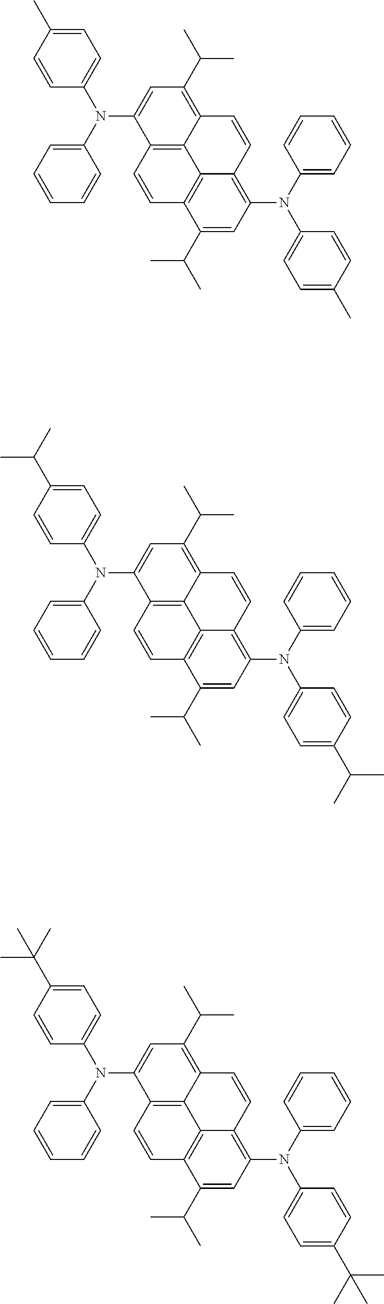

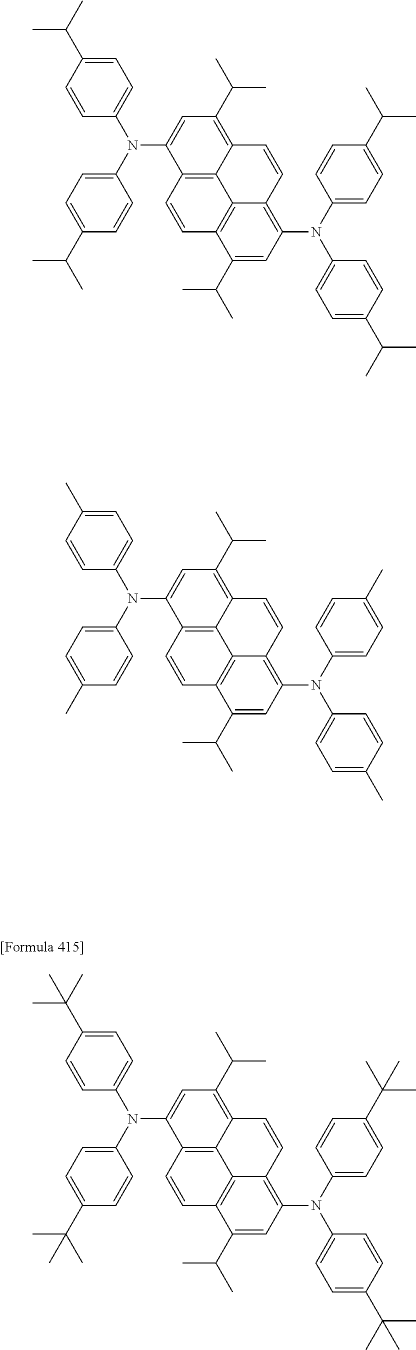

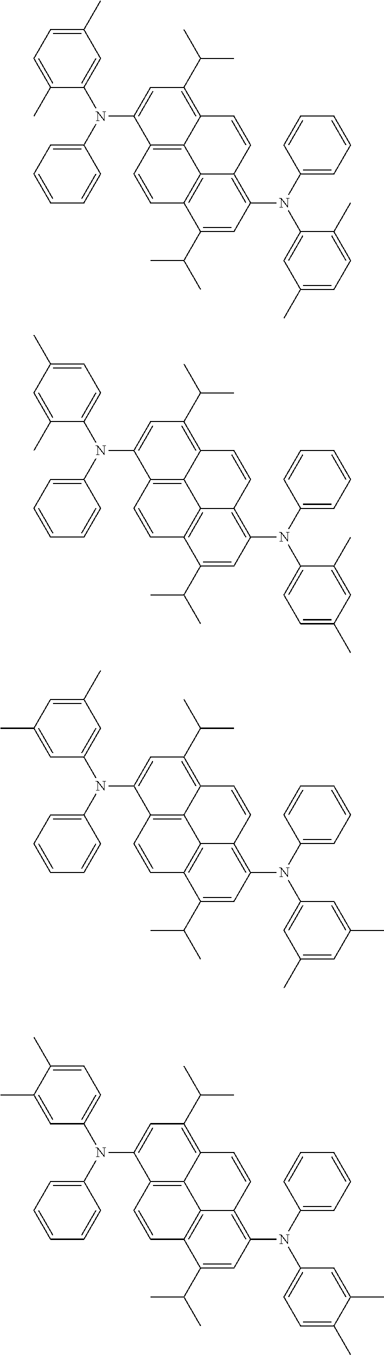

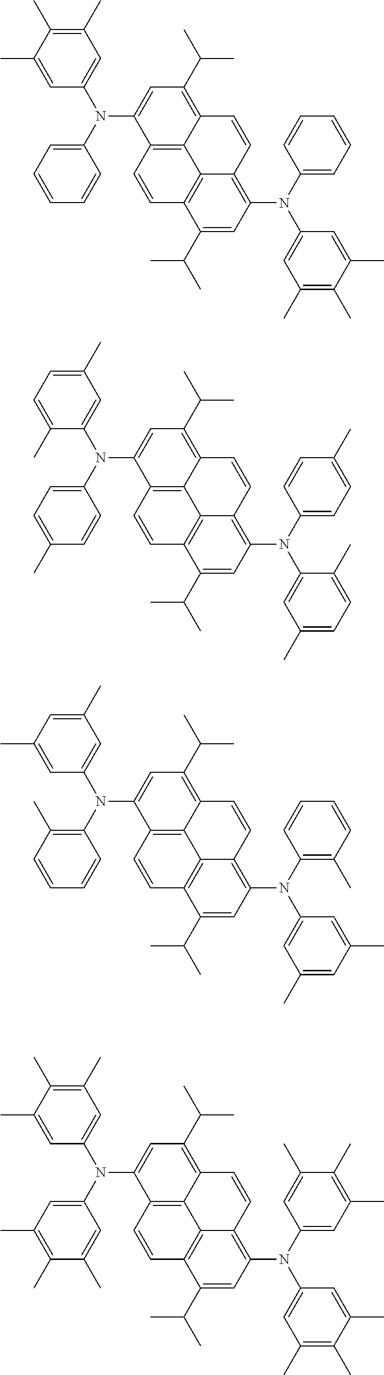

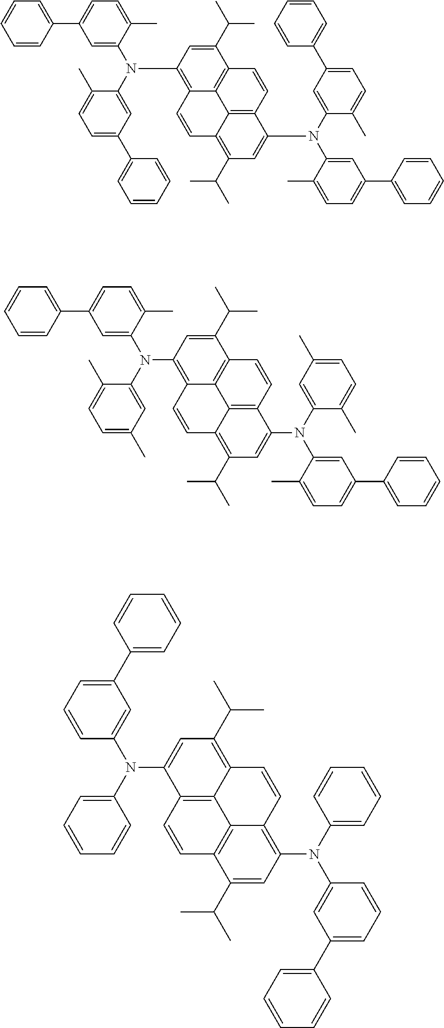 [SMILES: Cc1ccc(-c2ccccc2)cc1N(c1cc(-c2ccccc2)ccc1C)c1cc(C(C)C)c2ccc3c(N(c4cc(-c5ccccc5)ccc4C)c4cc(-c5ccccc5)ccc4C)cc(C(C)C)c4ccc1c2c43 ; Cc1ccc(C)c(N(c2cc(-c3ccccc3)ccc2C)c2cc(C(C)C)c3ccc4c(N(c5cc(C)ccc5C)c5cc(-c6ccccc6)ccc5C)cc(C(C)C)c5ccc2c3c54)c1 ; CC(C)c1cc(N(c2ccccc2)c2cccc(-c3ccccc3)c2)c2ccc3c(C(C)C)cc(N(c4ccccc4)c4cccc(-c5ccccc5)c4)c4ccc1c2c34]

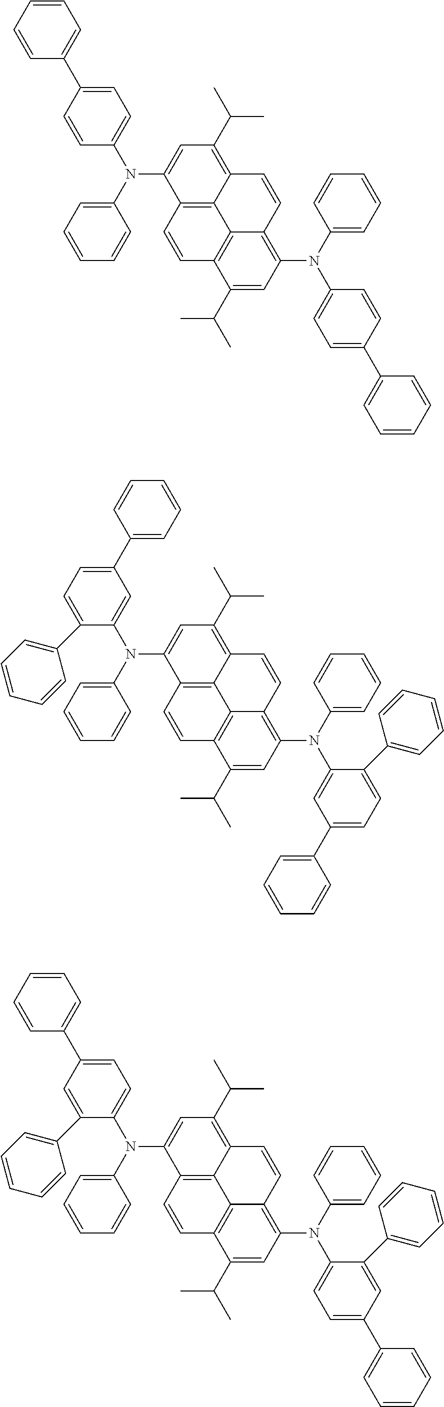 [SMILES: CC(C)c1cc(N(c2ccccc2)c2ccc(-c3ccccc3)cc2)c2ccc3c(C(C)C)cc(N(c4ccccc4)c4ccc(-c5ccccc5)cc4)c4ccc1c2c34 ; CC(C)c1cc(N(c2ccccc2)c2cc(-c3ccccc3)ccc2-c2ccccc2)c2ccc3c(C(C)C)cc(N(c4ccccc4)c4cc(-c5ccccc5)ccc4-c4ccccc4)c4ccc1c2c34 ; CC(C)c1cc(N(c2ccccc2)c2ccc(-c3ccccc3)cc2-c2ccccc2)c2ccc3c(C(C)C)cc(N(c4ccccc4)c4ccc(-c5ccccc5)cc4-c4ccccc4)c4ccc1c2c34]

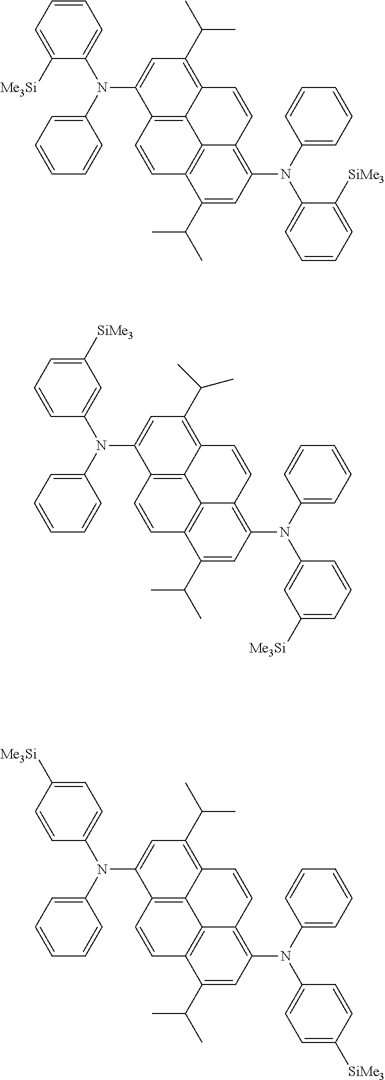 [SMILES: CC(C)c1cc(N(c2ccccc2)c2ccccc2[Si](C)(C)C)c2ccc3c(C(C)C)cc(N(c4ccccc4)c4ccccc4[Si](C)(C)C)c4ccc1c2c34 ; CC(C)c1cc(N(c2ccccc2)c2cccc([Si](C)(C)C)c2)c2ccc3c(C(C)C)cc(N(c4ccccc4)c4cccc([Si](C)(C)C)c4)c4ccc1c2c34 ; CC(C)c1cc(N(c2ccccc2)c2ccc([Si](C)(C)C)cc2)c2ccc3c(C(C)C)cc(N(c4ccccc4)c4ccc([Si](C)(C)C)cc4)c4ccc1c2c34]

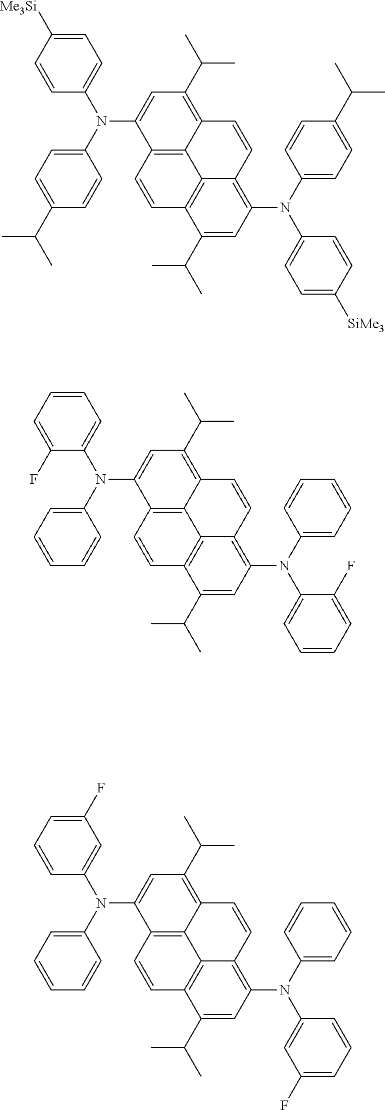 [SMILES: CC(C)c1ccc(N(c2ccc([Si](C)(C)C)cc2)c2cc(C(C)C)c3ccc4c(N(c5ccc(C(C)C)cc5)c5ccc([Si](C)(C)C)cc5)cc(C(C)C)c5ccc2c3c54)cc1 ; CC(C)c1cc(N(c2ccccc2)c2ccccc2F)c2ccc3c(C(C)C)cc(N(c4ccccc4)c4ccccc4F)c4ccc1c2c34 ; CC(C)c1cc(N(c2ccccc2)c2cccc(F)c2)c2ccc3c(C(C)C)cc(N(c4ccccc4)c4cccc(F)c4)c4ccc1c2c34]

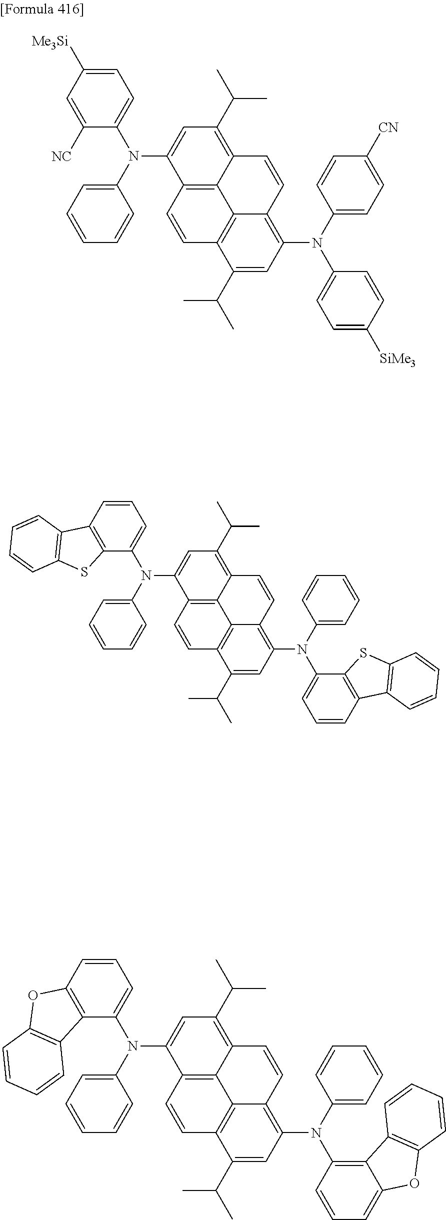 [SMILES: CC(C)c1cc(N(c2ccc(C#N)cc2)c2ccc([Si](C)(C)C)cc2)c2ccc3c(C(C)C)cc(N(c4ccccc4)c4ccc([Si](C)(C)C)cc4C#N)c4ccc1c2c34 ; CC(C)c1cc(N(c2ccccc2)c2cccc3c2sc2ccccc23)c2ccc3c(C(C)C)cc(N(c4ccccc4)c4cccc5c4sc4ccccc45)c4ccc1c2c34 ; CC(C)c1cc(N(c2ccccc2)c2cccc3oc4ccccc4c23)c2ccc3c(C(C)C)cc(N(c4ccccc4)c4cccc5oc6ccccc6c45)c4ccc1c2c34]

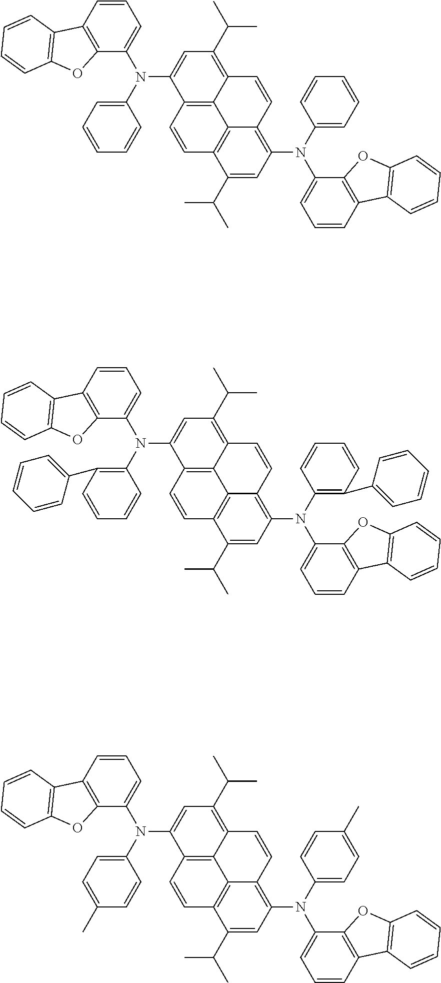 [SMILES: CC(C)c1cc(N(c2ccccc2)c2cccc3c2oc2ccccc23)c2ccc3c(C(C)C)cc(N(c4ccccc4)c4cccc5c4oc4ccccc45)c4ccc1c2c34 ; CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34 ; Cc1ccc(N(c2cc(C(C)C)c3ccc4c(N(c5ccc(C)cc5)c5cccc6c5oc5ccccc56)cc(C(C)C)c5ccc2c3c54)c2cccc3c2oc2ccccc23)cc1]

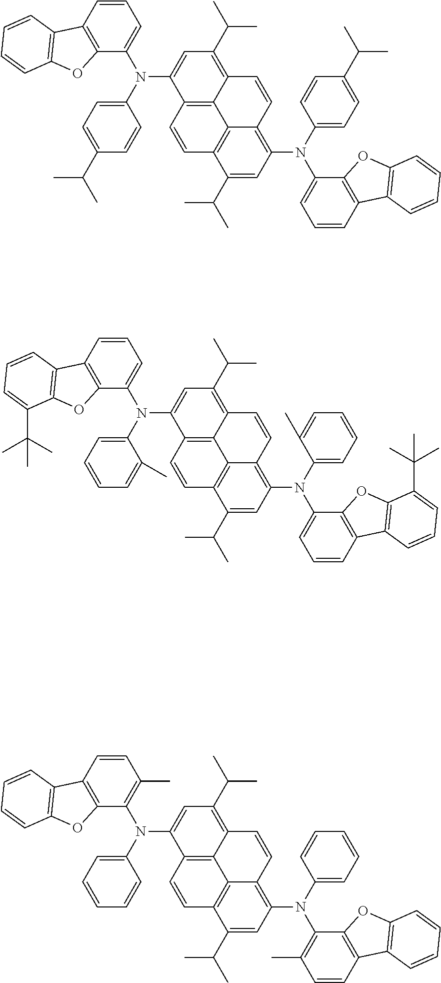 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; Cc1ccccc1N(c1cc(C(C)C)c2ccc3c(N(c4ccccc4C)c4cccc5c4oc4c(C(C)(C)C)cccc45)cc(C(C)C)c4ccc1c2c43)c1cccc2c1oc1c(C(C)(C)C)cccc12 ; Cc1ccc2c(oc3ccccc32)c1N(c1ccccc1)c1cc(C(C)C)c2ccc3c(N(c4ccccc4)c4c(C)ccc5c4oc4ccccc45)cc(C(C)C)c4ccc1c2c43]

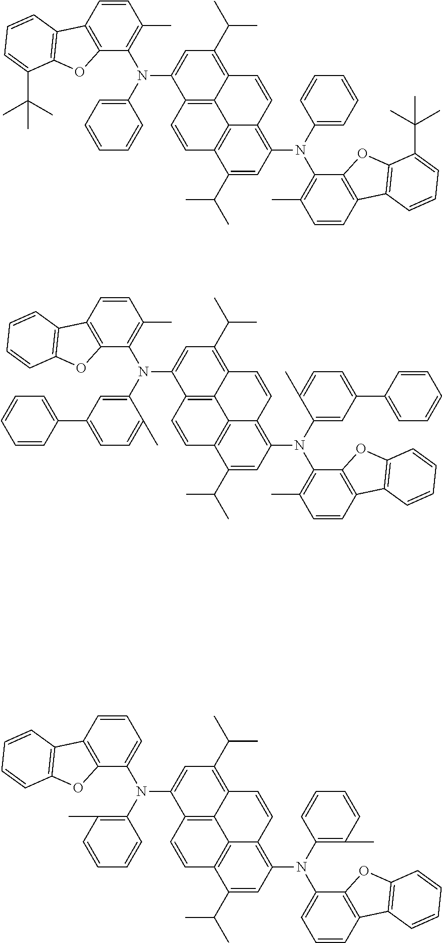 [SMILES: Cc1ccc2c(oc3c(C(C)(C)C)cccc32)c1N(c1ccccc1)c1cc(C(C)C)c2ccc3c(N(c4ccccc4)c4c(C)ccc5c4oc4c(C(C)(C)C)cccc45)cc(C(C)C)c4ccc1c2c43 ; Cc1ccc(-c2ccccc2)cc1N(c1cc(C(C)C)c2ccc3c(N(c4cc(-c5ccccc5)ccc4C)c4c(C)ccc5c4oc4ccccc45)cc(C(C)C)c4ccc1c2c43)c1c(C)ccc2c1oc1ccccc12 ; Cc1ccccc1N(c1cc(C(C)C)c2ccc3c(N(c4ccccc4C)c4cccc5c4oc4ccccc45)cc(C(C)C)c4ccc1c2c43)c1cccc2c1oc1ccccc12]

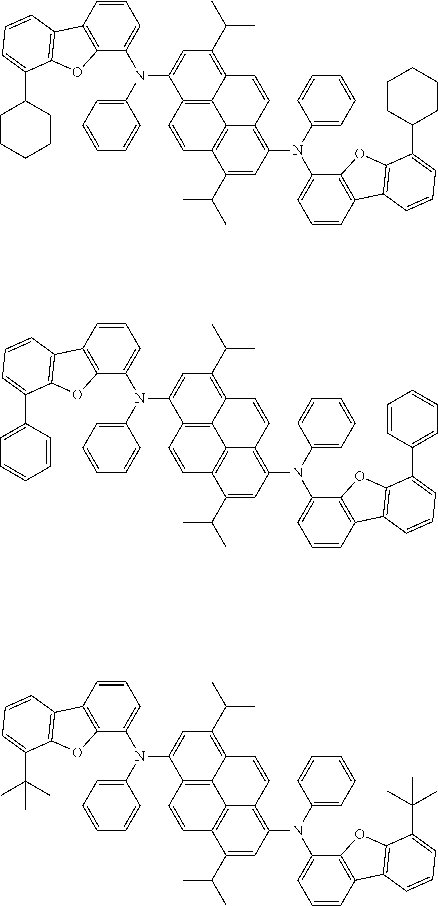 [SMILES: CC(C)c1cc(N(c2ccccc2)c2cccc3c2oc2c(C4CCCCC4)cccc23)c2ccc3c(C(C)C)cc(N(c4ccccc4)c4cccc5c4oc4c(C6CCCCC6)cccc45)c4ccc1c2c34 ; CC(C)c1cc(N(c2ccccc2)c2cccc3c2oc2c(-c4ccccc4)cccc23)c2ccc3c(C(C)C)cc(N(c4ccccc4)c4cccc5c4oc4c(-c6ccccc6)cccc45)c4ccc1c2c34 ; CC(C)c1cc(N(c2ccccc2)c2cccc3c2oc2c(C(C)(C)C)cccc23)c2ccc3c(C(C)C)cc(N(c4ccccc4)c4cccc5c4oc4c(C(C)(C)C)cccc45)c4ccc1c2c34]

### Compound Represented by Formula (4)

The compound represented by the formula (4) will be described below.

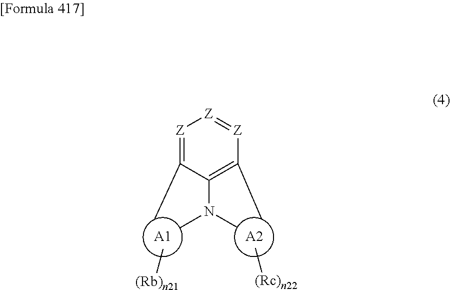

In the formula (4):
Z are each independently CRa or a nitrogen atom;
a ring A1 and a ring A2 are each independently a substituted or unsubstituted aromatic hydrocarbon ring having 6 to 50 ring carbon atoms, or a substituted or unsubstituted heterocycle having 5 to 50 ring atoms;
when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded;
n₂₁ and n₂₂ are each independently 0, 1, 2, 3 or 4;
when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded;
when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and
Ra, Rb, and Rc forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the ring A1 and the ring A2 has the same structure as a compound formed by introducing a hydrogen atom to the “aryl group” described above.

Ring atoms of the “aromatic hydrocarbon ring” for the ring A1 and the ring A2 include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the ring A1 and the ring A2 has the same structure as a compound formed by introducing a hydrogen atom to the “heterocyclic group” described above.

Ring atoms of the “heterocycle” for the ring A1 and the ring A2 include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring as the ring A1 or any one of atoms forming the heterocycle as the ring A1.

Rc is bonded to any one of carbon atoms forming the aromatic hydrocarbon ring as the ring A2 or any one of atoms forming the heterocycle as the ring A2.

At least one of Ra, Rb, or Rc is preferably a group represented by a formula (4a) below. More preferably, at least two of Ra, Rb, or Rc are each a group represented by the formula (4a).

[Formula 418]

*-L₄₀₁-Ar₄₀₁ (4a)

In the formula (4a):
L₄₀₁ is a single bond, a substituted or unsubstituted arylene group having 6 to 30 ring carbon atoms, or a substituted or unsubstituted divalent heterocyclic group having 5 to 30 ring atoms; and
Ar₄₀₁ is a substituted or unsubstituted aryl group having 6 to 50 ring carbon atoms, a substituted or unsubstituted heterocyclic group having 5 to 50 ring atoms, or a group represented by a formula (4b) below.

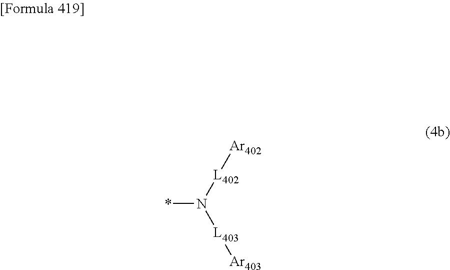

In the formula (4b):
L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms;
a combination of Ar₄₀₂ and Ar₄₀₃ are mutually bonded to form a substituted or unsubstituted monocyclic ring, mutually bonded to form a substituted or unsubstituted fused ring, or not mutually bonded; and
Ar₄₀₂ and Ar₄₀₃ forming neither the substituted or unsubstituted monocyclic ring nor the substituted or unsubstituted fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

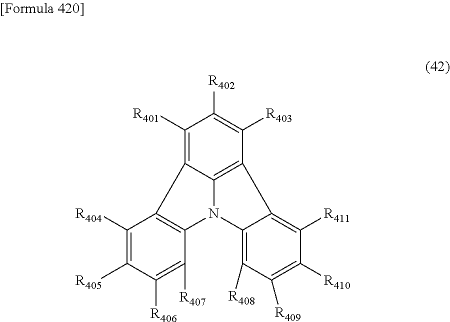

In the formula (42):
at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and
R₄₀₁ to R₄₁₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a). More preferably, at least two of R₄₀₁ to R₄₁₁ are each a group represented by the formula (4a).

R₄₀₄ and R₄₁₁ are each preferably a group represented by the formula (4a).

In an exemplary embodiment, the compound represented by the formula (4) is a compound formed by bonding a structure represented by a formula (4-1) or a formula (4-2) below to the ring A1.

Further, in an exemplary embodiment, the compound represented by the formula (42) is a compound formed by bonding a structure represented by the formula (4-1) or the formula (4-2) to a ring bonded to R₄₀₄ to R₄₀₇.

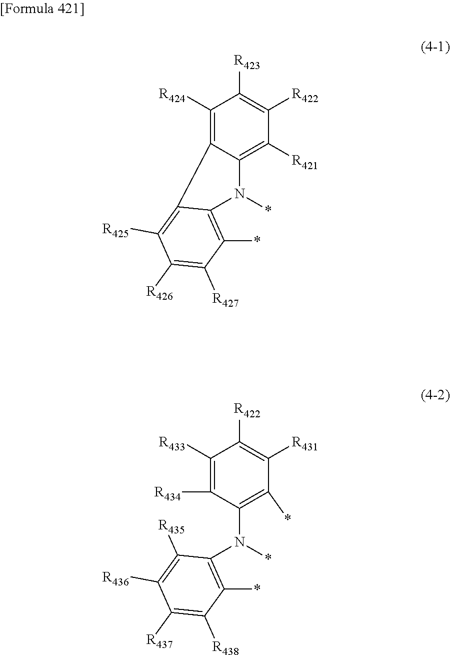

In the formula (4-1), two * are each independently bonded to a ring carbon atom of the aromatic hydrocarbon ring or a ring atom of the heterocycle as the A1 ring in the formula (4) or bonded to one of R₄₀₄ to R₄₀₇ in the formula (42);
in the formula (4-2), three * are each independently bonded to a ring carbon atom of the aromatic hydrocarbon ring or a ring atom of the heterocycle as the A1 ring in the formula (4) or bonded to one of R₄₀₄ to R₄₀₇ in the formula (42);
at least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded;
at least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded; and
R₄₂₁ to R₄₂₇ and R₄₃₁ to R₄₃₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), a formula (41-4) or a formula (41-5) below.

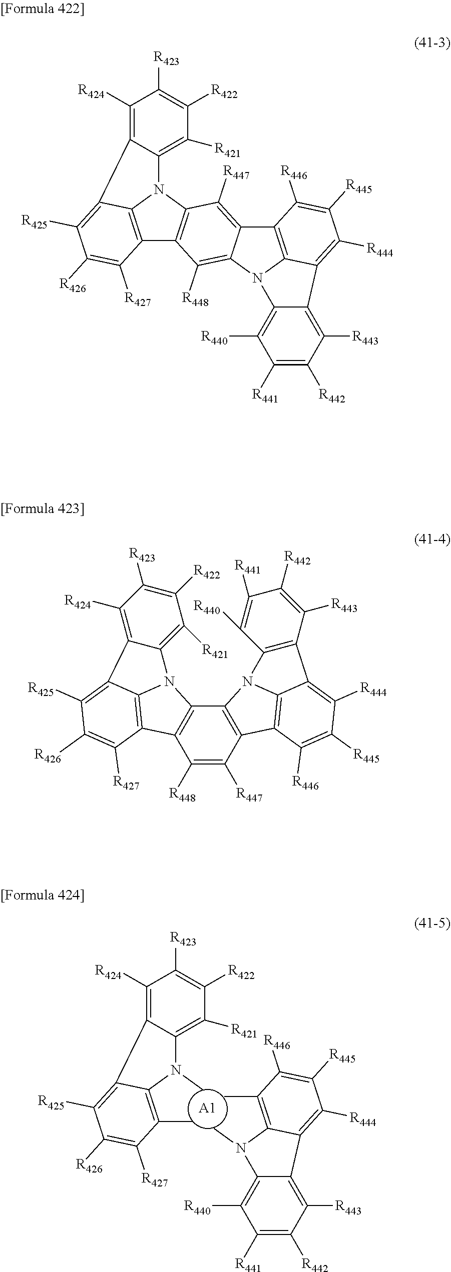

In the formulae (41-3), (41-4), and (41-5):
a ring A1 is as defined in the formula (4);
R₄₂₁ to R₄₂₇ each independently represent the same as R₄₂₁ to R₄₂₇ in the formula (4-1); and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms as the A1 ring in the formula (41-5) is a substituted or unsubstituted naphthalene ring or a substituted or unsubstituted fluorene ring.

In an exemplary embodiment, a substituted or unsubstituted heterocycle having 5 to 50 ring atoms as the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is selected from the group consisting of compounds represented by formulae (461) to (467) below.

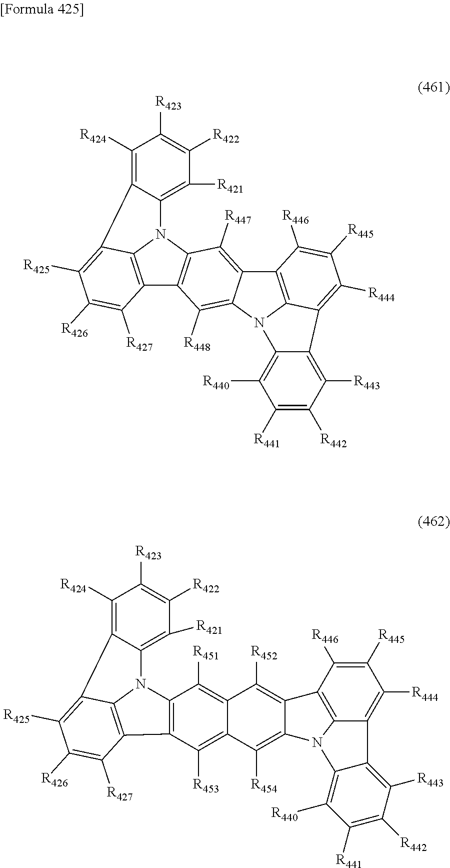

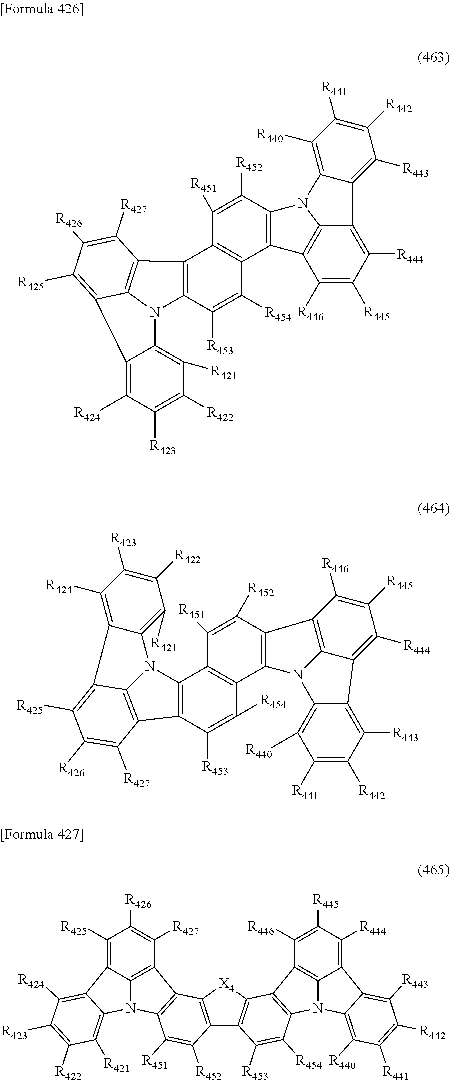

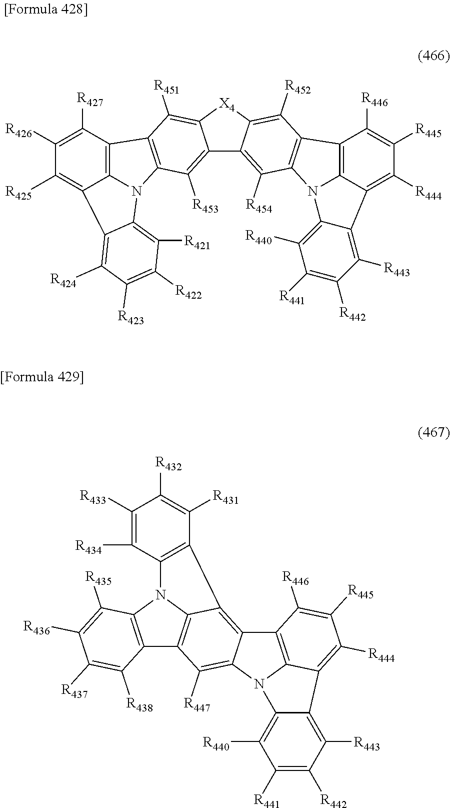

In the formulae (461), (462), (463), (464), (465), (466), and (467):
R₄₂₁ to R₄₂₇ each independently represent the same as R₄₂₁ to R₄₂₇ in the formula (4-1);
R₄₃₁ to R₄₃₈ each independently represent the same as R₄₃₁ to R₄₃₈ in the formula (4-2);
R₄₄₀ to R₄₄₈ and R₄₅₁ to R₄₅₄ each independently represent the same as R₄₀₁ to R₄₁₁ in the formula (42);
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; preferably, a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, at least one combination of adjacent two or more of R₄₀₁ to R₄₁₁ in the compound represented by the formula (42) are mutually bonded to form a substituted or unsubstituted monocyclic ring, or mutually bonded to form a substituted or unsubstituted fused ring. In this exemplary embodiment, the compound represented by the formula (42) will be described in detail below as a compound represented by a formula (45) below.

### Compound Represented by Formula (45)

The compound represented by the formula (45) will be described below.

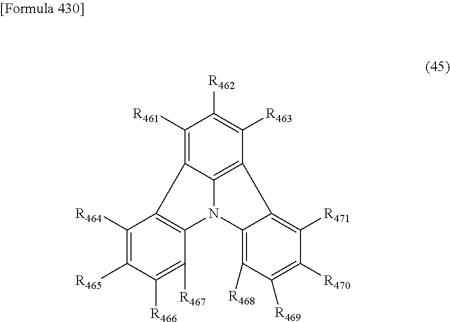

In the formula (45): two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring.

Here, the combination of R₄₆₁ and R₄₆₂ and the combination of R₄₆₂ and R₄₆₃; the combination of R₄₆₄ and R₄₆₅ and the combination of R₄₆₅ and R₄₆₆; the combination of R₄₆₅ and R₄₆₆ and the combination of R₄₆₆ and R₄₆₇; the combination of R₄₆₈ and R₄₆₉ and the combination of R₄₆₉ and R₄₇₀; and the combination of R₄₆₉ and R₄₇₀ and the combination of R₄₇₀ and R₄₇₁ do not simultaneously form a ring;

the two or more rings formed by R₄₆₁ to R₄₇₁ are mutually the same or different; and
R₄₆₁ to R₄₇₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring carbon atoms bonded to Rₙ and Rₙ₊₁. The ring is preferably formed of atoms selected from the group consisting of a carbon atom, an oxygen atom, a sulfur atom, and a nitrogen atom, and is made of preferably 3 to 7 atoms, and more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on the corresponding one of the three benzene rings of the formula (45).

Examples of the above cyclic structures in the compound represented by the formula (45) include structures represented by formulae (451) to (460) below.

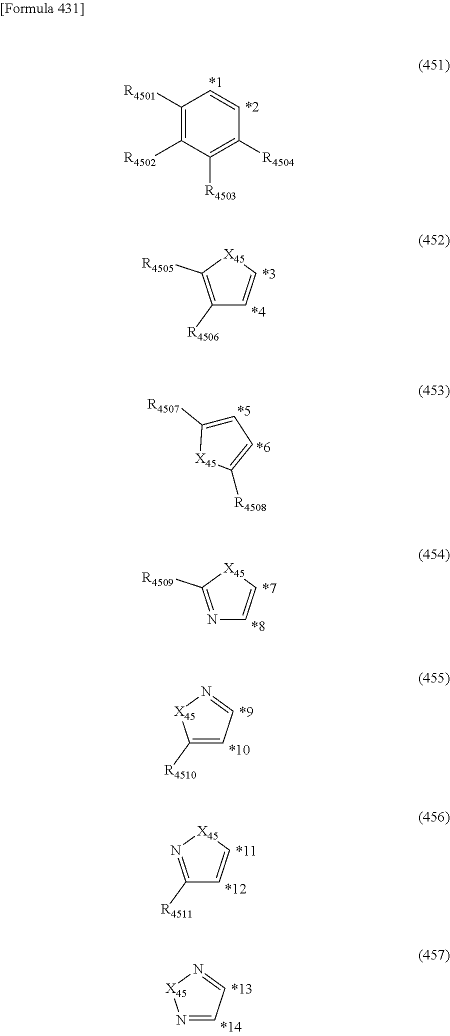

In the formulae (451) to (457):
each combination of *1 and *2, *3 and *4, *5 and *6, *7 and *8, *9 and *10, *11 and *12, and *13 and *14 represents the two ring carbon atoms bonded to Rₙ and Rₙ+1;

the ring carbon atom bonded to Rₙ may be any one of the two ring carbon atoms represented by *1 and *2, *3 and *4, *5 and *6, *7 and *8, *9 and *10, *11 and *12, and *13 and *14;
X₄₅ is C(R₄₅₁₂)(R₄₅₁₃), NR₄₅₁₄, an oxygen atom, or a sulfur atom;
at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and
R₄₅₀₁ to R₄₅₁₄ forming neither the substituted or unsubstituted monocyclic ring nor the substituted or unsubstituted fused ring each independently represent the same as R₄₆₁ to R₄₇₁ in the formula (45).

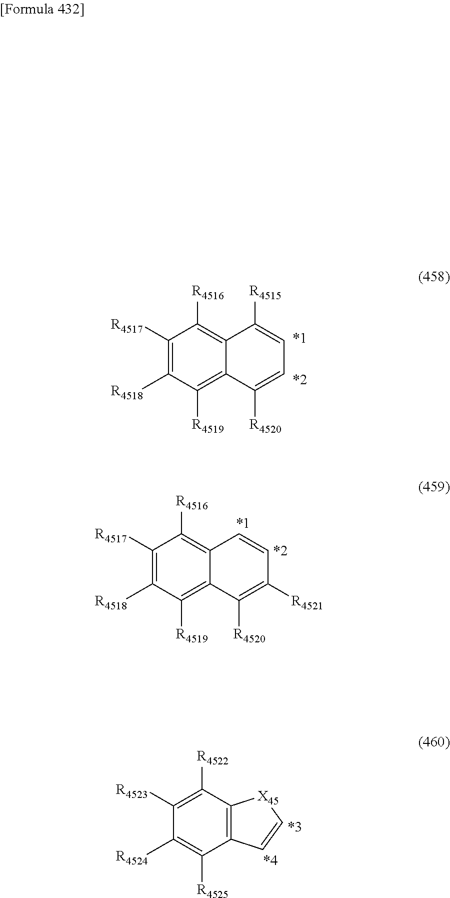

In the formulae (458) to (460):
each combination of *1 and *2, and *3 and *4 represents the two ring carbon atoms bonded to Rₙ and Rₙ+1;
the ring carbon atom bonded to Rₙ may be any one of the two ring carbon atoms represented by *1 and *2, or *3 and *4;
X₄₅ is C(R₄₅₁₂)(R₄₅₁₃), NR₄₅₁₄, an oxygen atom, or a sulfur atom;
at least one combination of adjacent two or more of R₄₅₁₂ to R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and
R₄₅₁₂ to R₄₅₁₃, R₄₅₁₅ to R₄₅₂₁, and R₄₅₂₂ to R₄₅₂₅ forming neither the monocyclic ring nor the a substituted or unsubstituted fused ring, and R₄₅₁₄ each independently represent the same as R₄₆₁ to R₄₇₁ in the formula (45).

In the formula (45), preferably, at least one of R₄₆₂, R₄₆₄, R₄₆₅, R₄₇₀ or R₄₇₁ (preferably, at least one of R₄₆₂, R₄₆₅ or R₄₇₀, more preferably R₄₆₂) is a group forming no cyclic structure.

(i) In the formula (45), a substituent, if present, for a cyclic structure formed by Rₙ and Rₙ₊₁, (ii) in the formula (45), R₄₆₁ to R₄₇₁ forming no cyclic structure, and (iii) R₄₅₀₁ to R₄₅₁₄, R₄₅₁₅ to R₄₅₂₅ in the formulae (451) to (460) are preferably each independently a group selected from the group consisting of a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464) below.

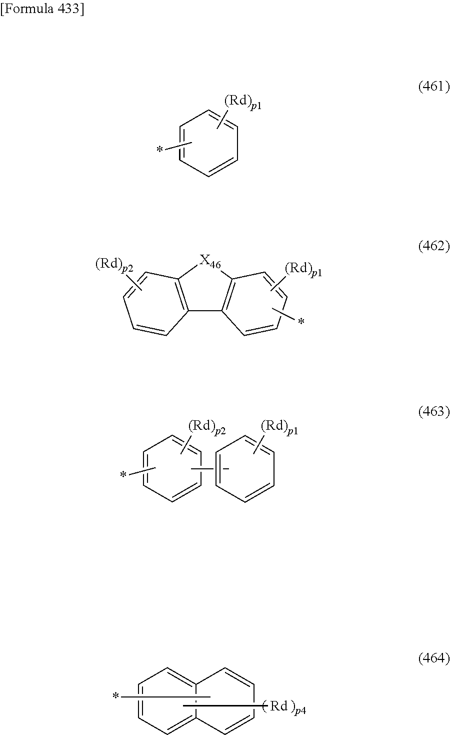

In the formulae (461) to (464):
R_{d} is each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄₆ is C(R₈₀₁)(R₈₀₂), NR₈₀₃, an oxygen atom, or a sulfur atom;
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; preferably, a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different;
when a plurality of R₈₀₃ are present, the plurality of R₈₀₃ are mutually the same or different;
p1 is 5;
p2 is 4;
p3 is 3;
p4 is 7; and
* in the formulae (461) to (464) each independently represent a bonding position to a cyclic structure.

In the luminescent compound, R₉₀₁ to R₉₀₇ are as defined above.

In an exemplary embodiment, the compound represented by the formula (45) is represented by one of formulae (45-1) to (45-6) below.

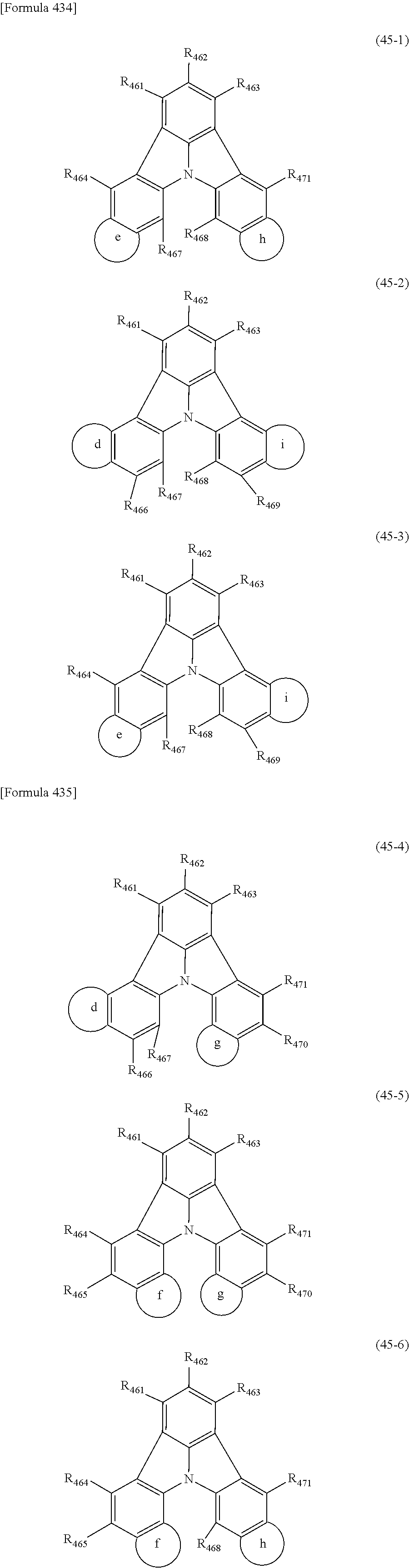

In the formulae (45-1) to (45-6):
rings d to i are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-7) to (45-12) below.

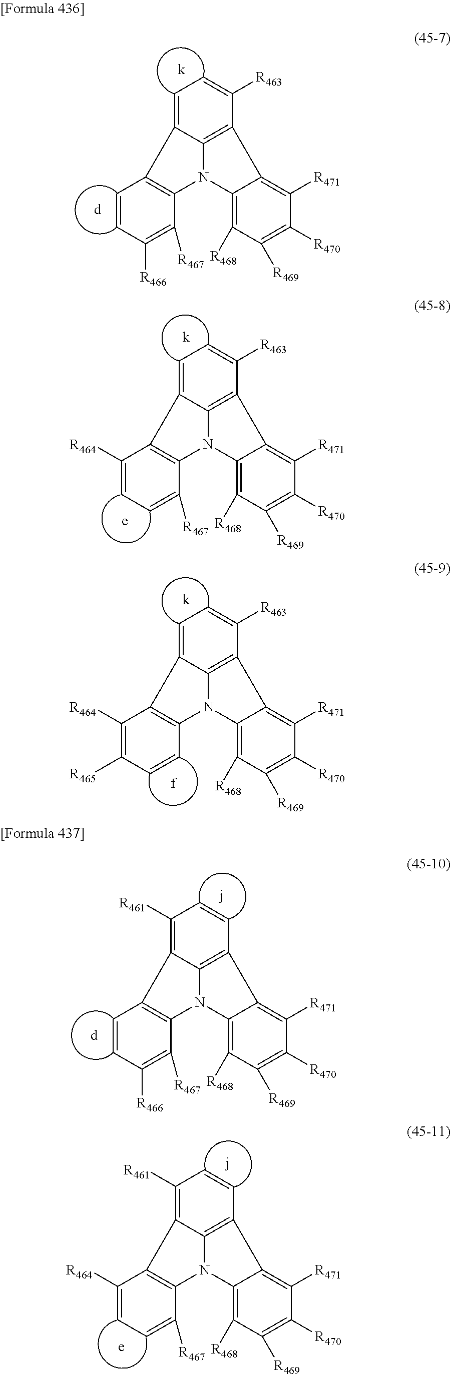

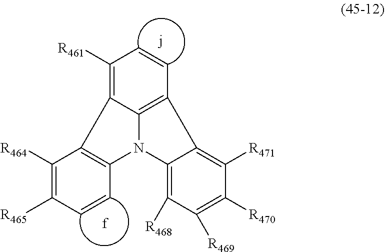

In the formulae (45-7) to (45-12):
rings d to f, k, and j are each independently a substituted or unsubstituted monocyclic ring or a substituted or unsubstituted fused ring; and
R₄₆₁ to R₄₇₁ each independently represent the same as R₄₈₁ to R₄₇₁ in the formula (45).

In an exemplary embodiment, the compound represented by the formula (45) is represented by one of formulae (45-13) to (45-21) below.

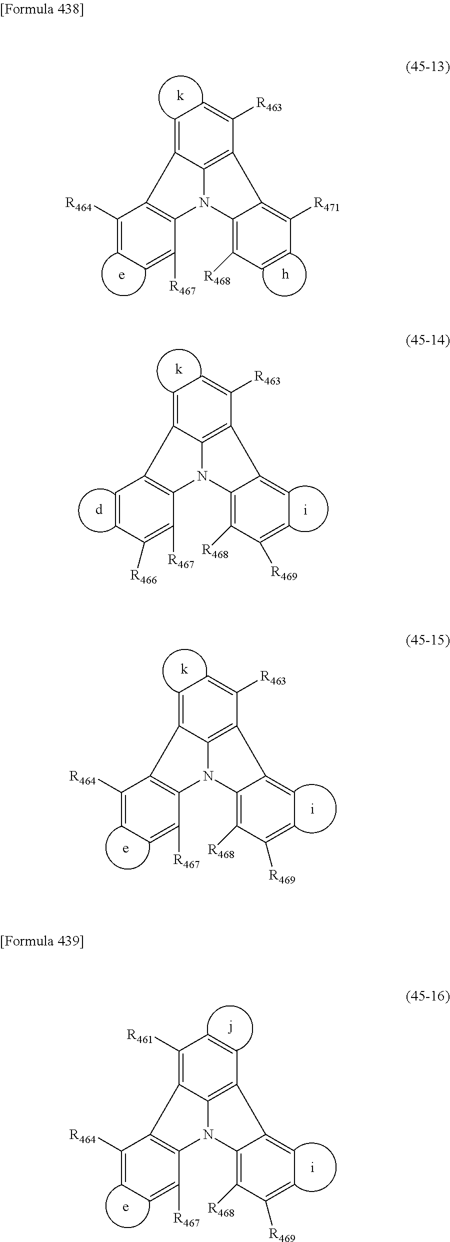

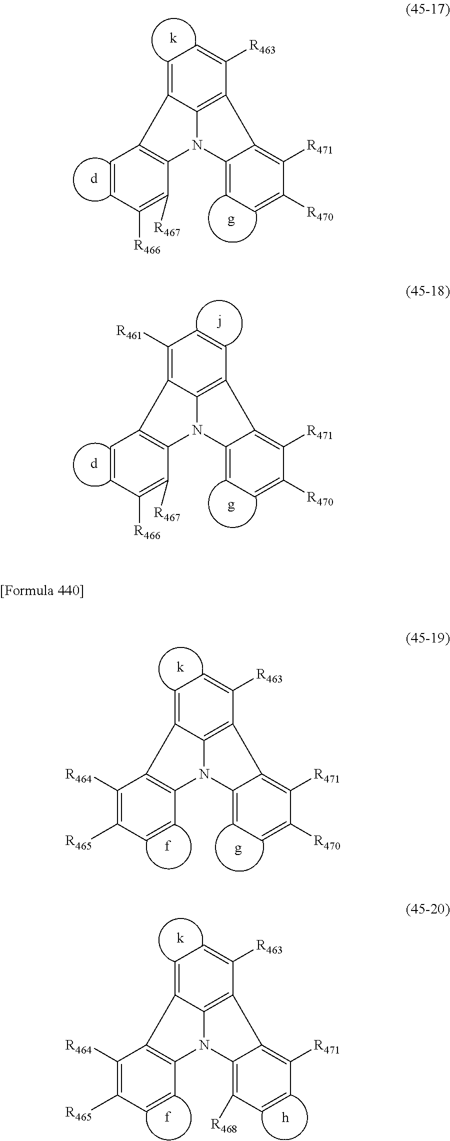

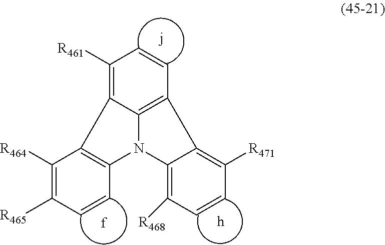

In the formulae (45-13) to (45-21):
rings d to k are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

When the ring g or the ring h each further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), and a group represented by the formula (464).

In an exemplary embodiment, the compound represented by the formula (45) is represented by one of formulae (45-22) to (45-25) below.

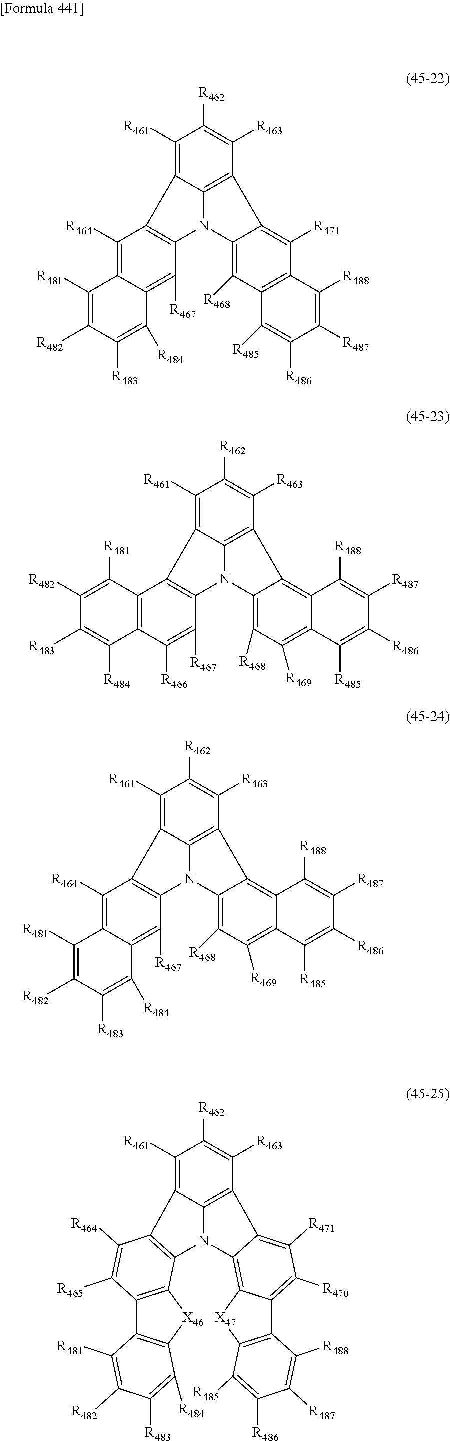

In the formulae (45-22) to (45-25):
X₄₆ and X₄₇ are each independently C(R₈₀₁)(R₈₀₂), NR₈₀₃, an oxygen atom, or a sulfur atom;
R₄₆₁ to R₄₇₁ and R₄₈₁ to R₄₈₈ each independently represent the same as R₄₆₁ to R₄₇₁ in the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; preferably, a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, the compound represented by the formula (45) is represented by a formula (45-26) below.

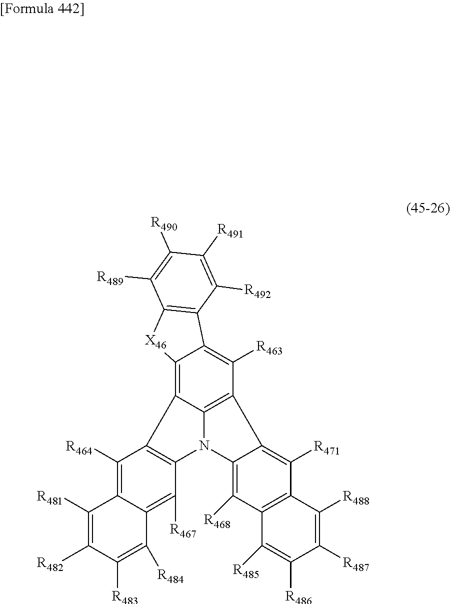

In the formula (45-26):
X₄₆ is C(R₈₀₁)(R₈₀₂), NR₈₀₃, an oxygen atom, or a sulfur atom;
R₄₆₃, R₄₆₄, R₄₆₇, R₄₆₈, R₄₇₁, and R₄₈₁ to R₄₉₂ each independently represent the same as R₄₆₁ to R₄₇₁ in the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; preferably, a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

### Specific Examples of Compound Represented by Formula (4)

Specific examples of the compound represented by the formula (4) include compounds as below. In the specific examples below, Ph represents a phenyl group, and D represents a deuterium atom.

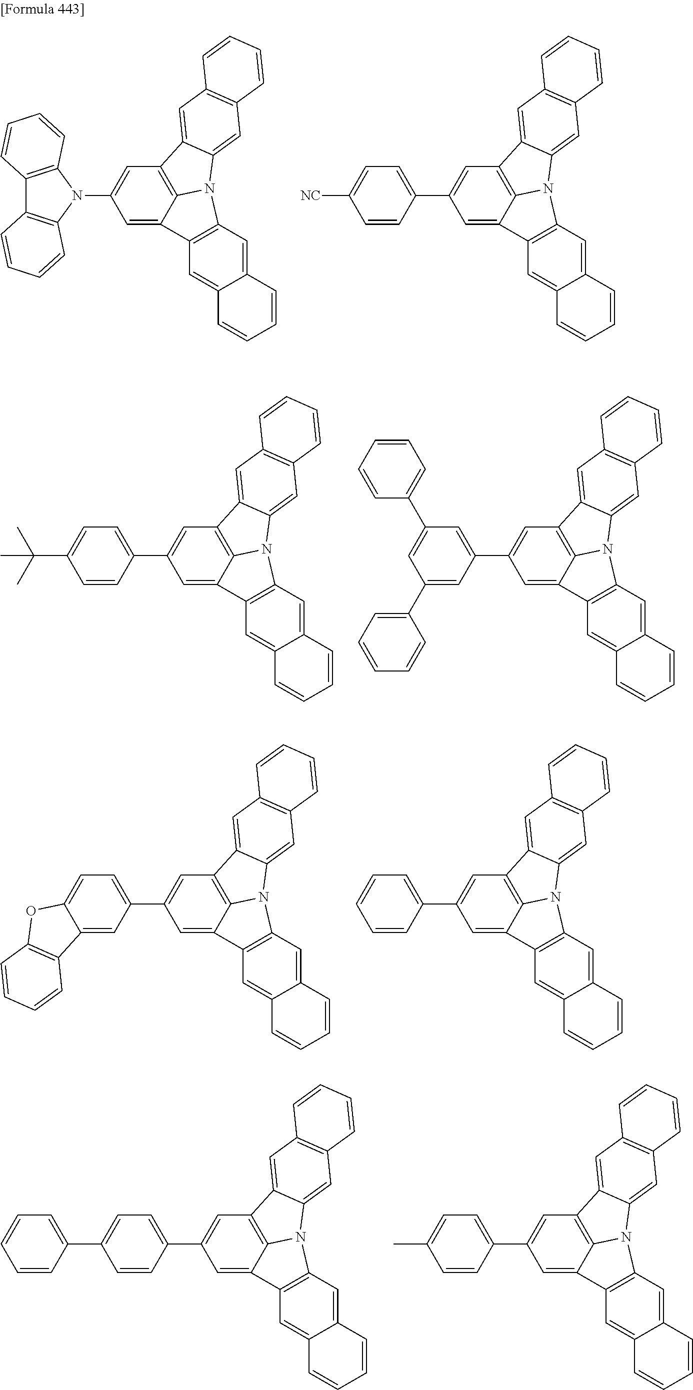

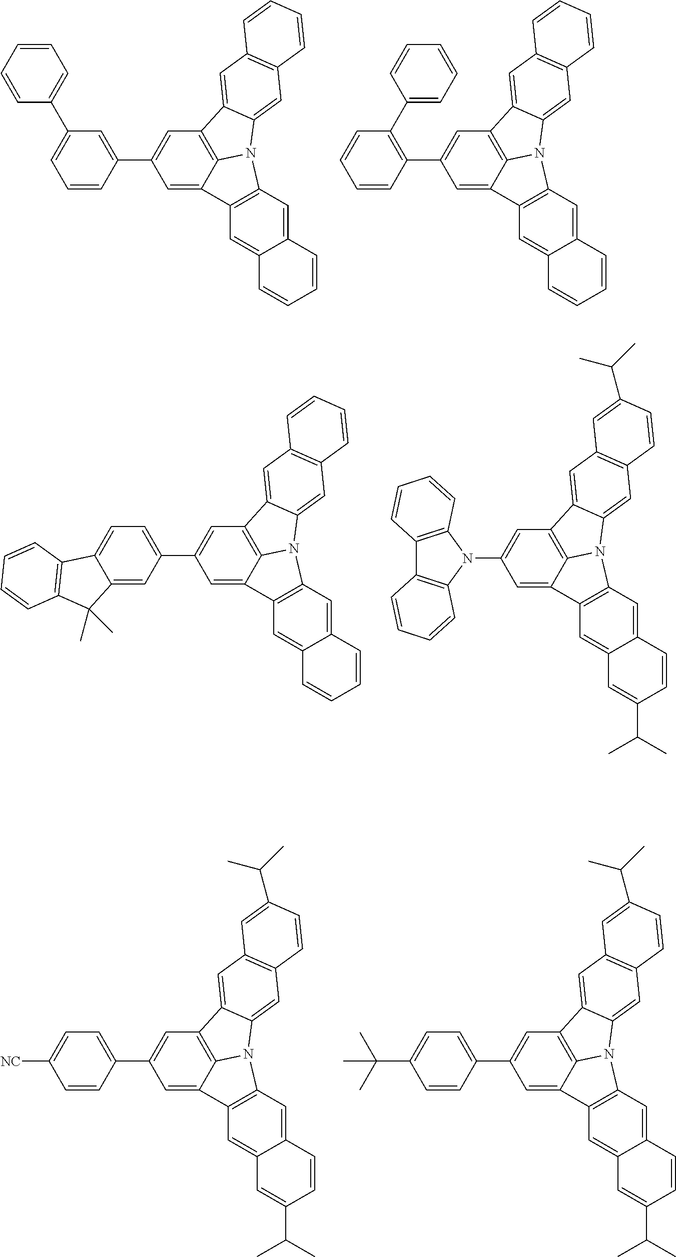

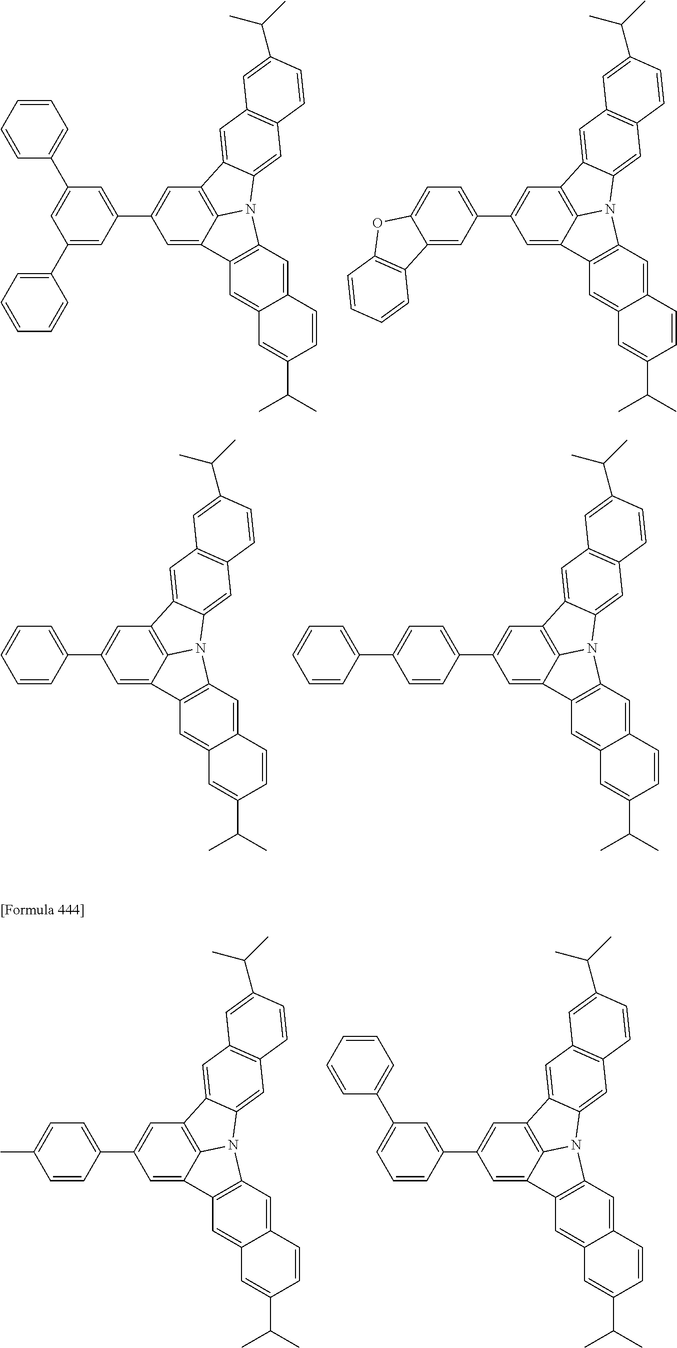

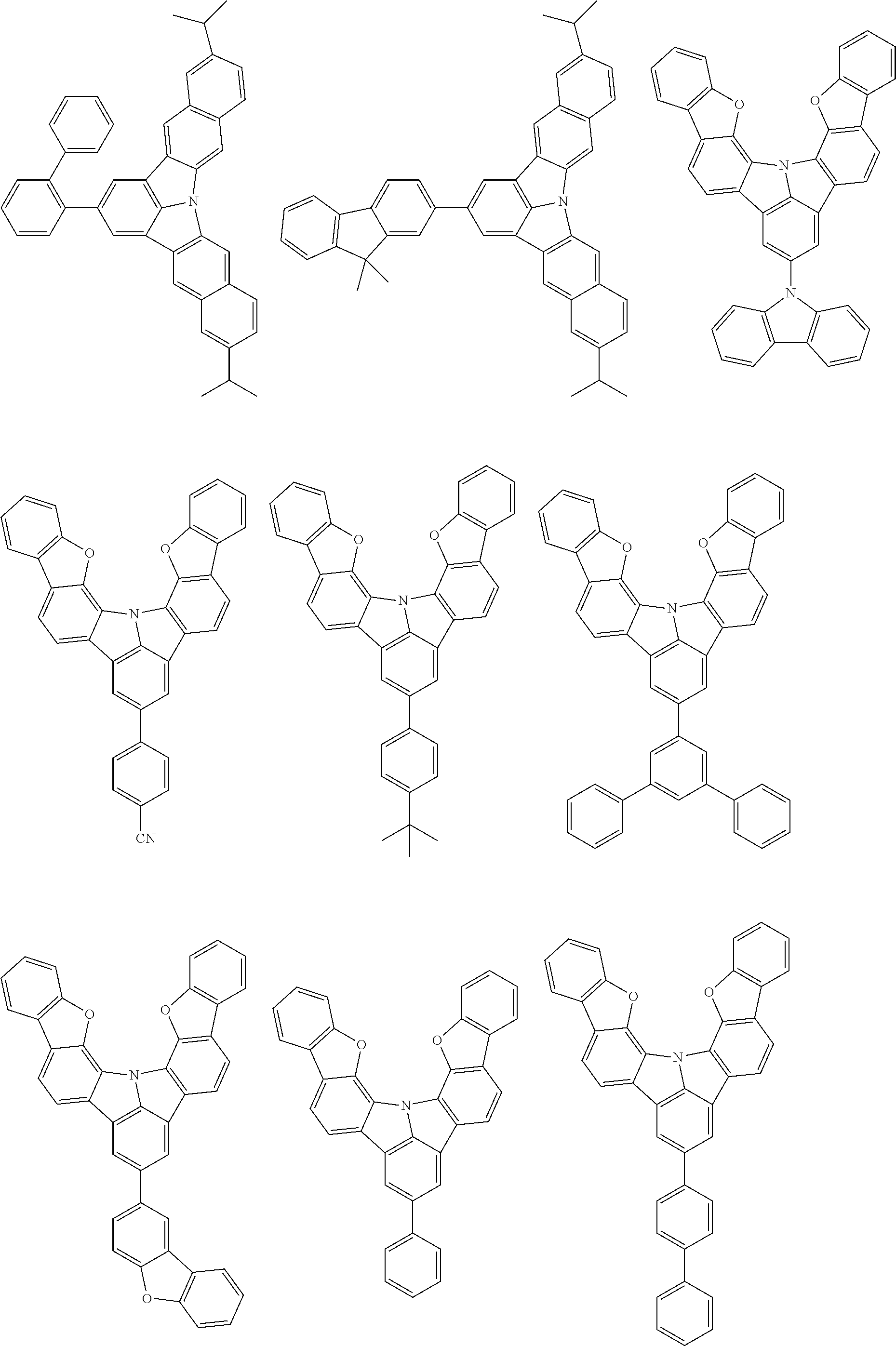

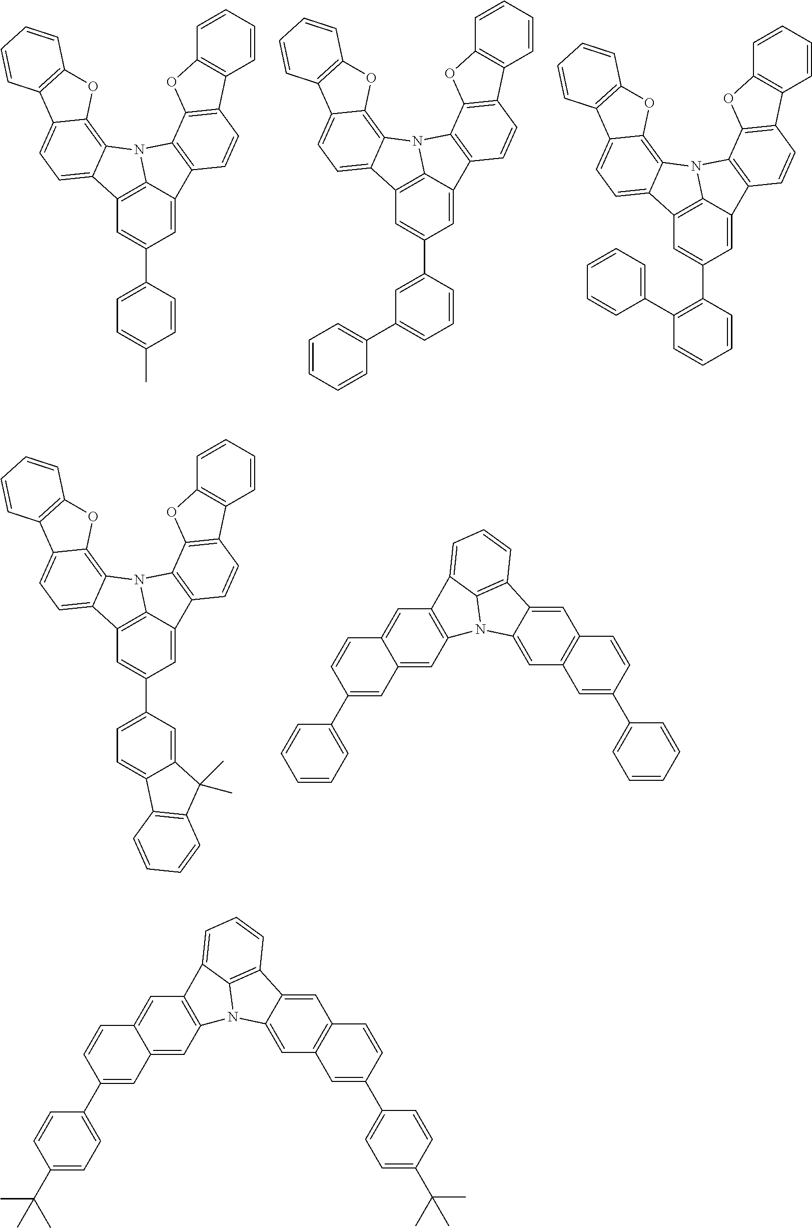

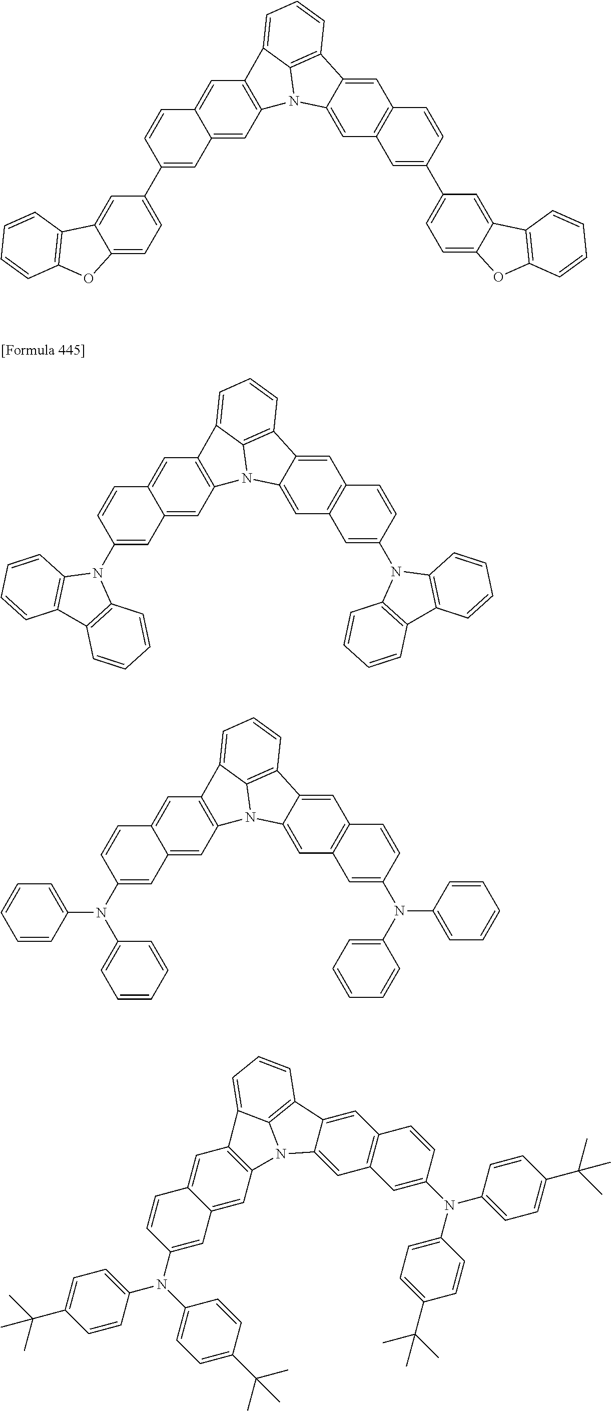 [SMILES: c1ccc2c(c1)oc1ccc(-c3ccc4cc5c6cccc7c8cc9ccc(-c%10ccc%11oc%12ccccc%12c%11c%10)cc9cc8n(c5cc4c3)c67)cc12 ; c1ccc2c(c1)c1ccccc1n2-c1ccc2cc3c4cccc5c6cc7ccc(-n8c9ccccc9c9ccccc98)cc7cc6n(c3cc2c1)c45 ; c1ccc(N(c2ccccc2)c2ccc3cc4c5cccc6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cccc6c7cc8ccc(N(c9ccc(C(C)(C)C)cc9)c9ccc(C(C)(C)C)cc9)cc8cc7n(c4cc3c2)c56)cc1]

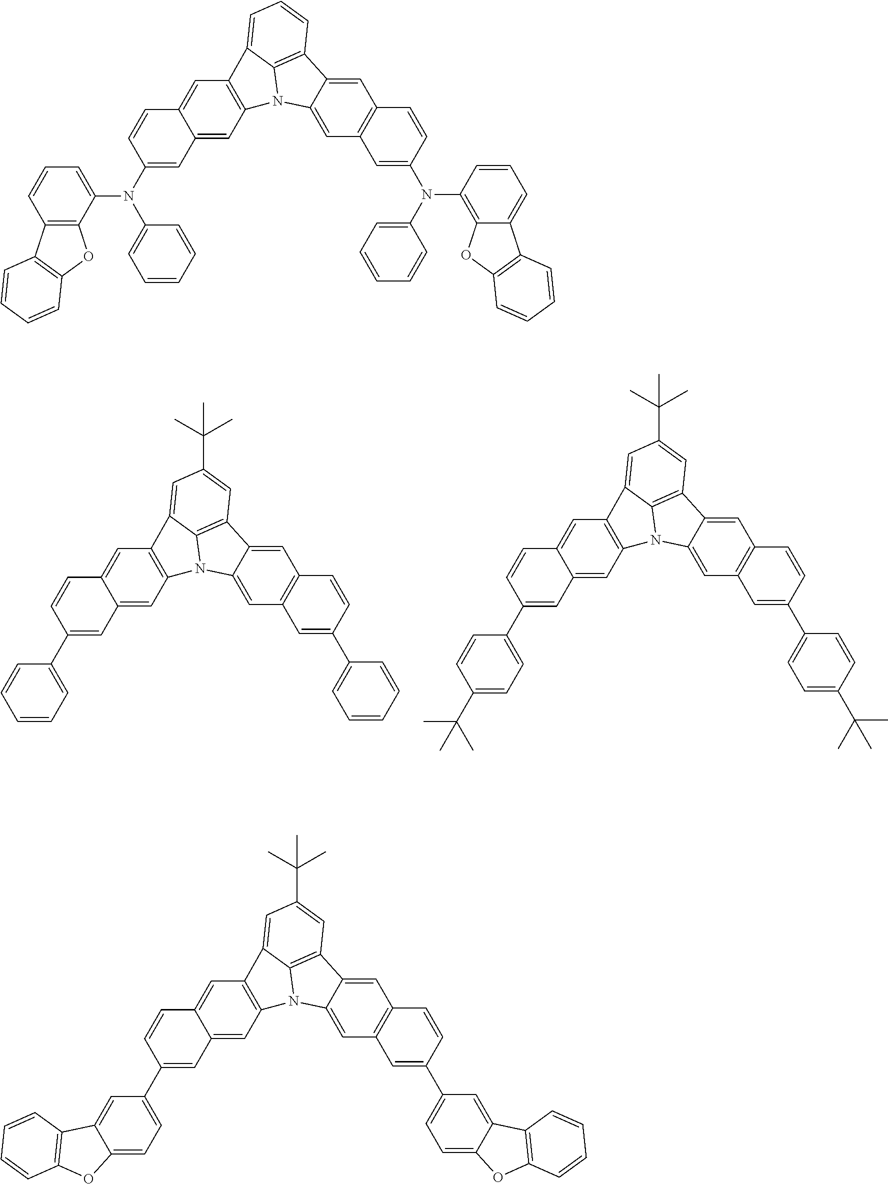

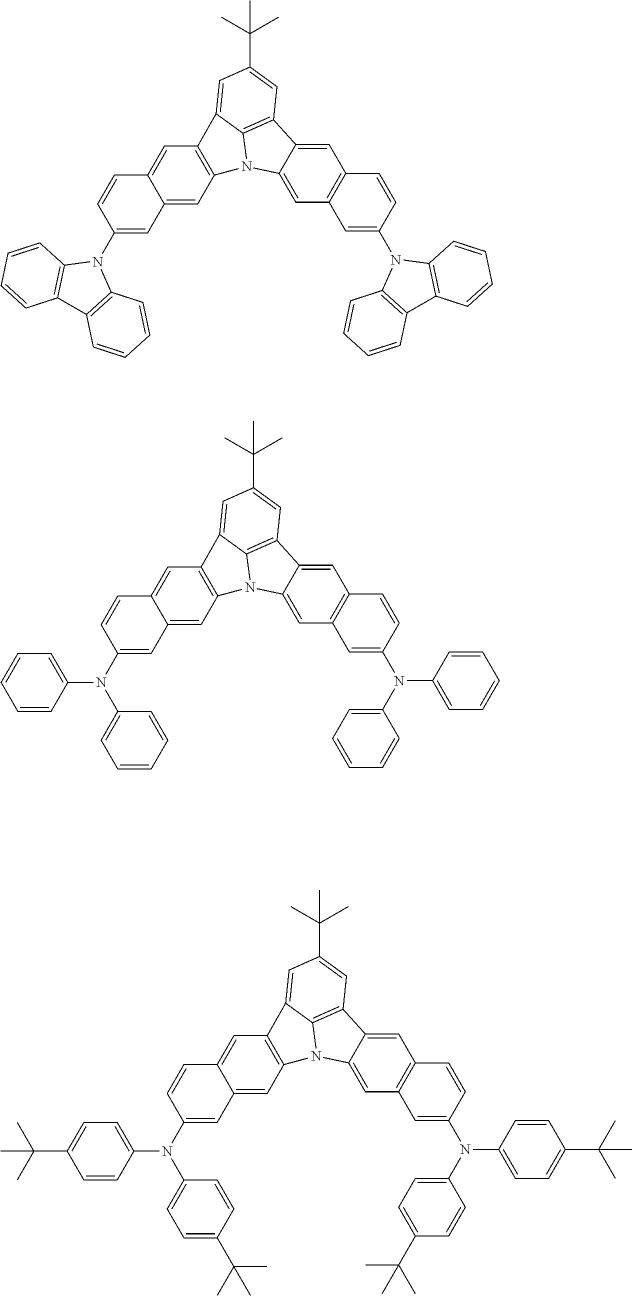 [SMILES: CC(C)(C)c1cc2c3cc4ccc(-n5c6ccccc6c6ccccc65)cc4cc3n3c4cc5cc(-n6c7ccccc7c7ccccc76)ccc5cc4c(c1)c23 ; CC(C)(C)c1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C(C)(C)C)cc6c7cc8ccc(N(c9ccc(C(C)(C)C)cc9)c9ccc(C(C)(C)C)cc9)cc8cc7n(c4cc3c2)c56)cc1]

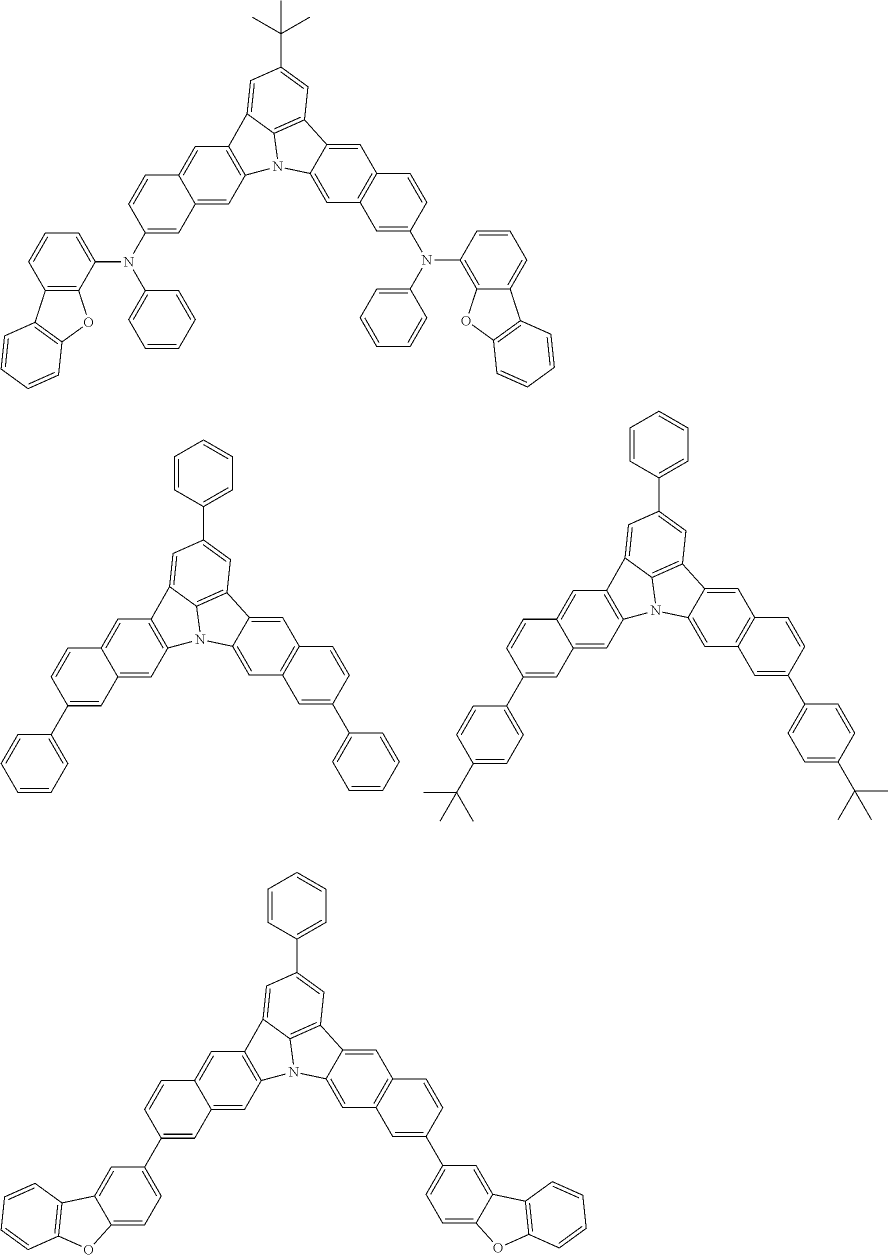

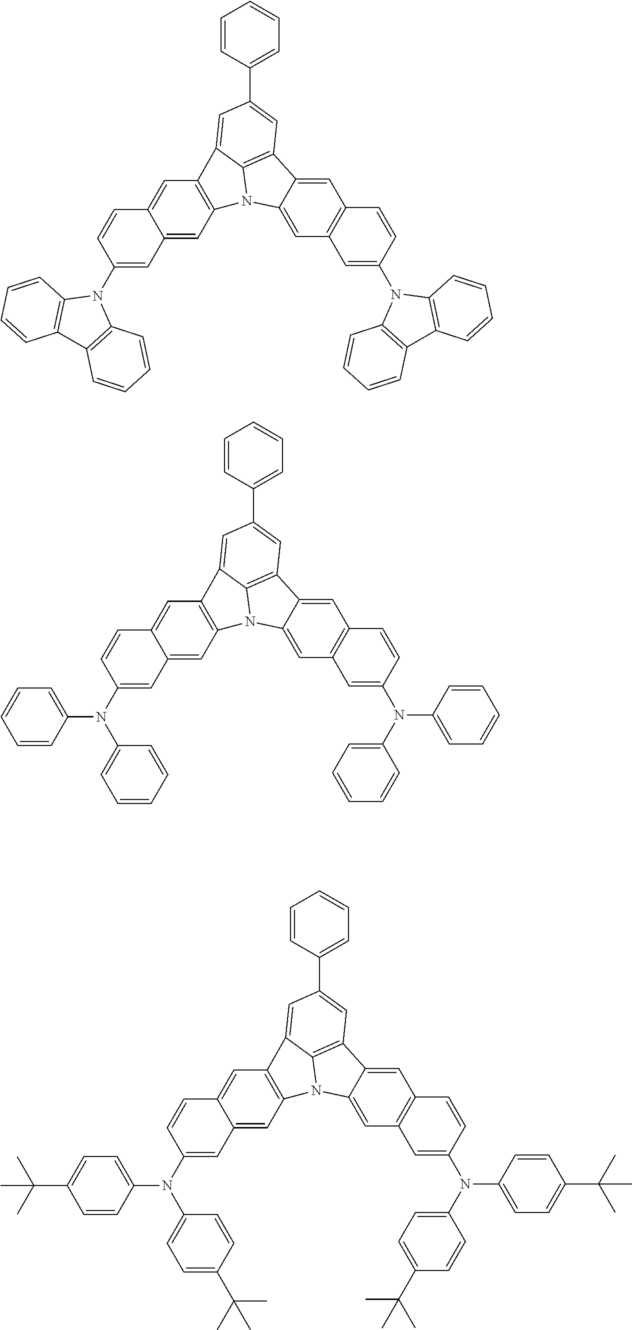 [SMILES: c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1 ; c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

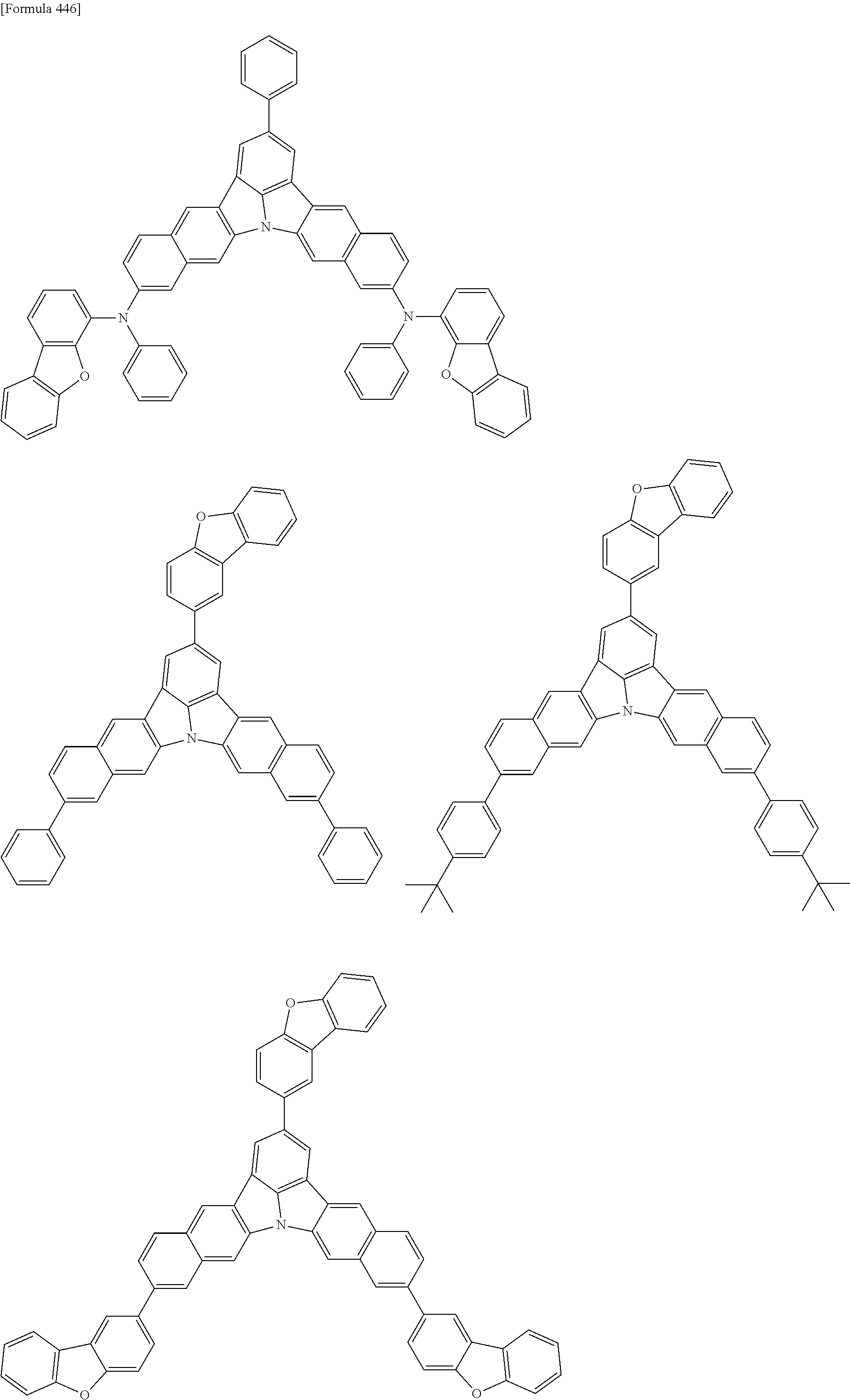

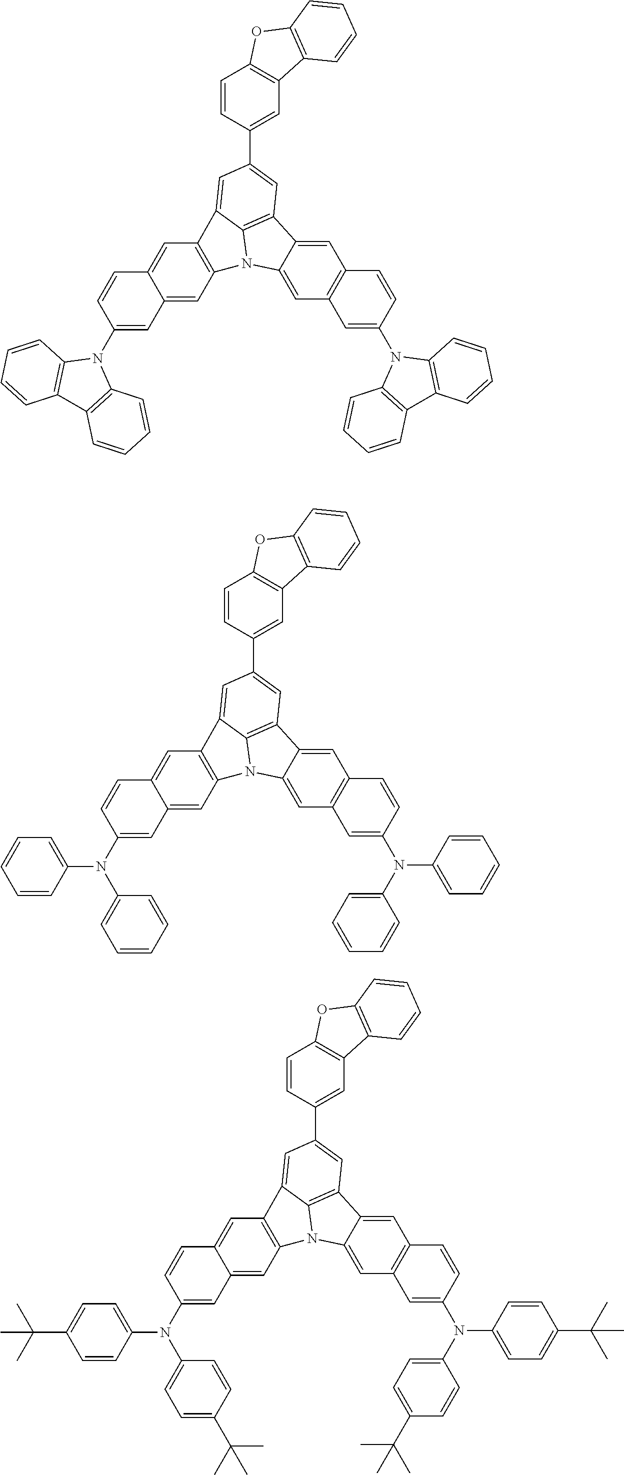

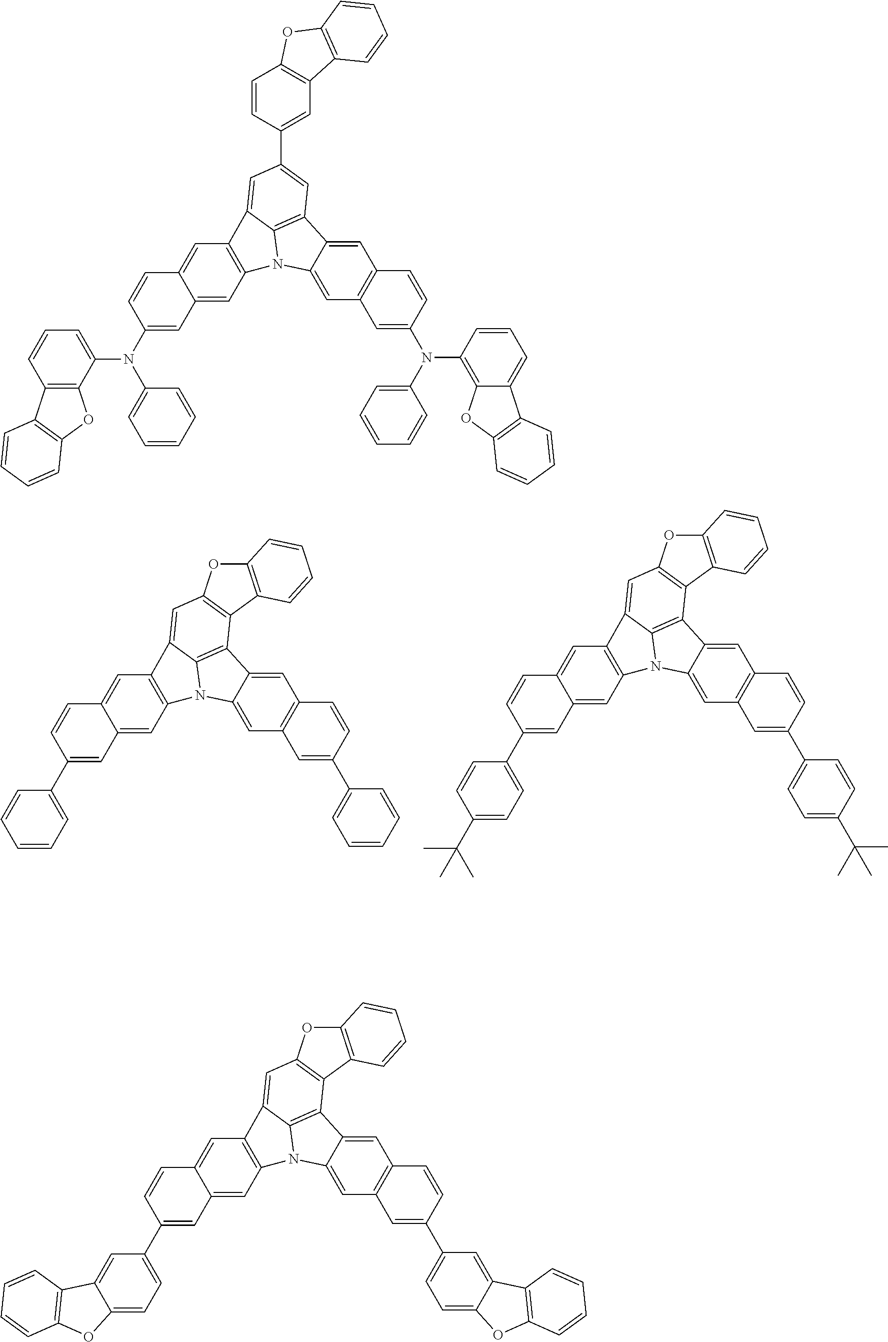

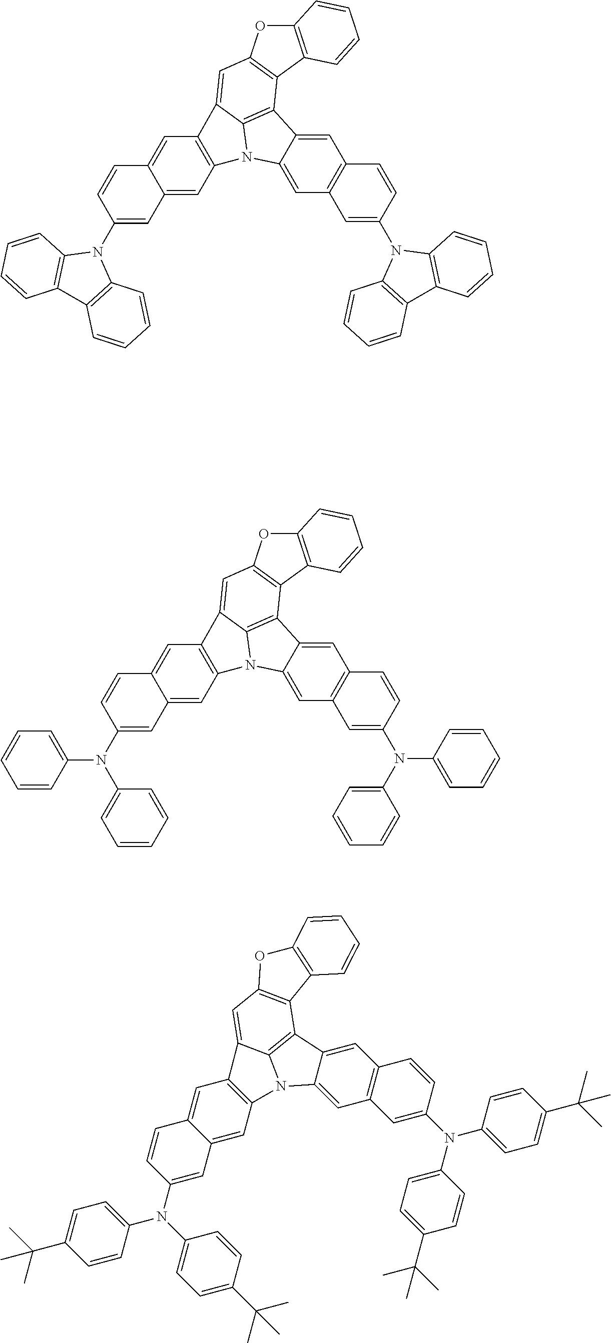 [SMILES: c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34 ; c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6oc7ccccc7c6c6c7cc8ccc(N(c9ccc(C(C)(C)C)cc9)c9ccc(C(C)(C)C)cc9)cc8cc7n(c4cc3c2)c56)cc1]

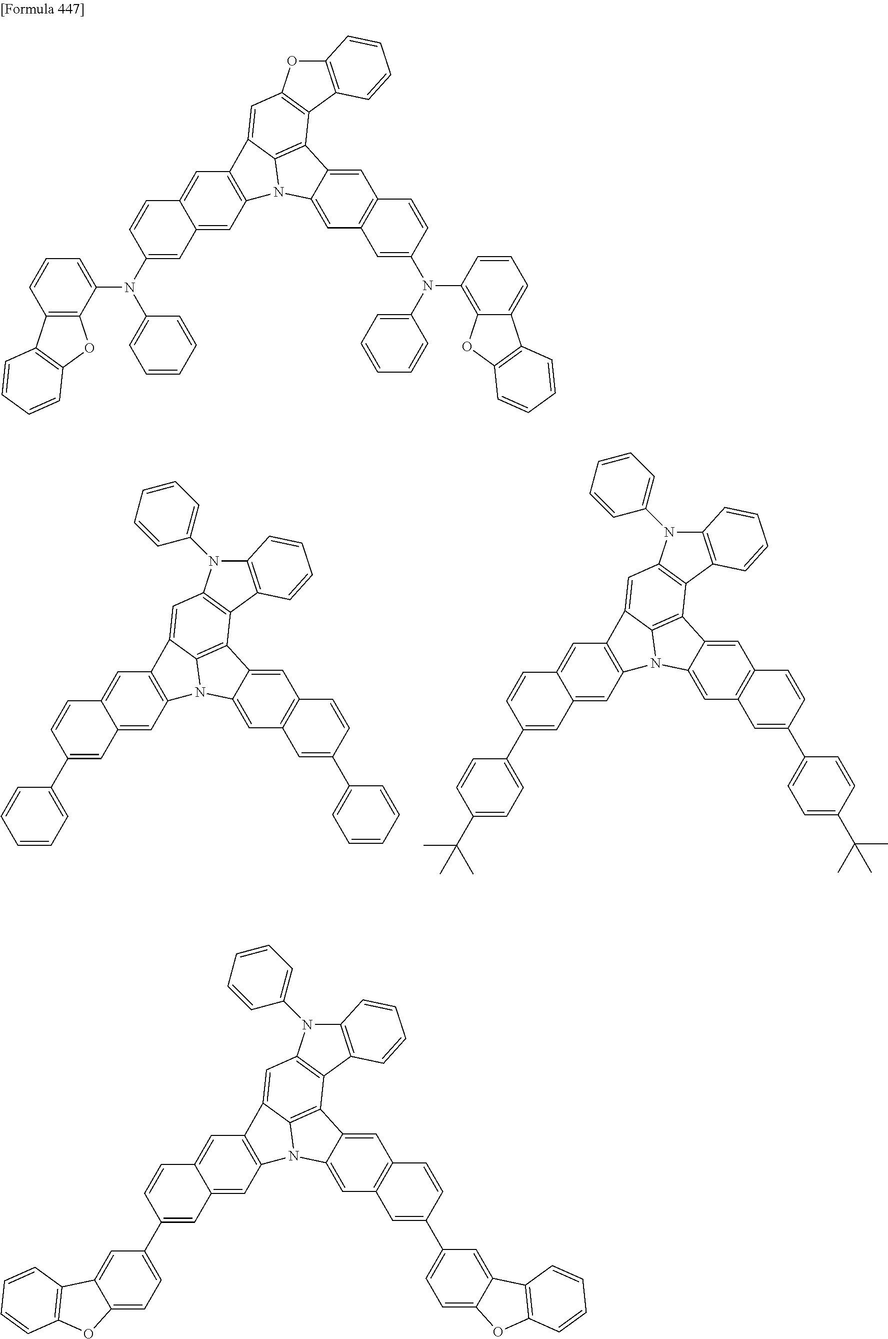

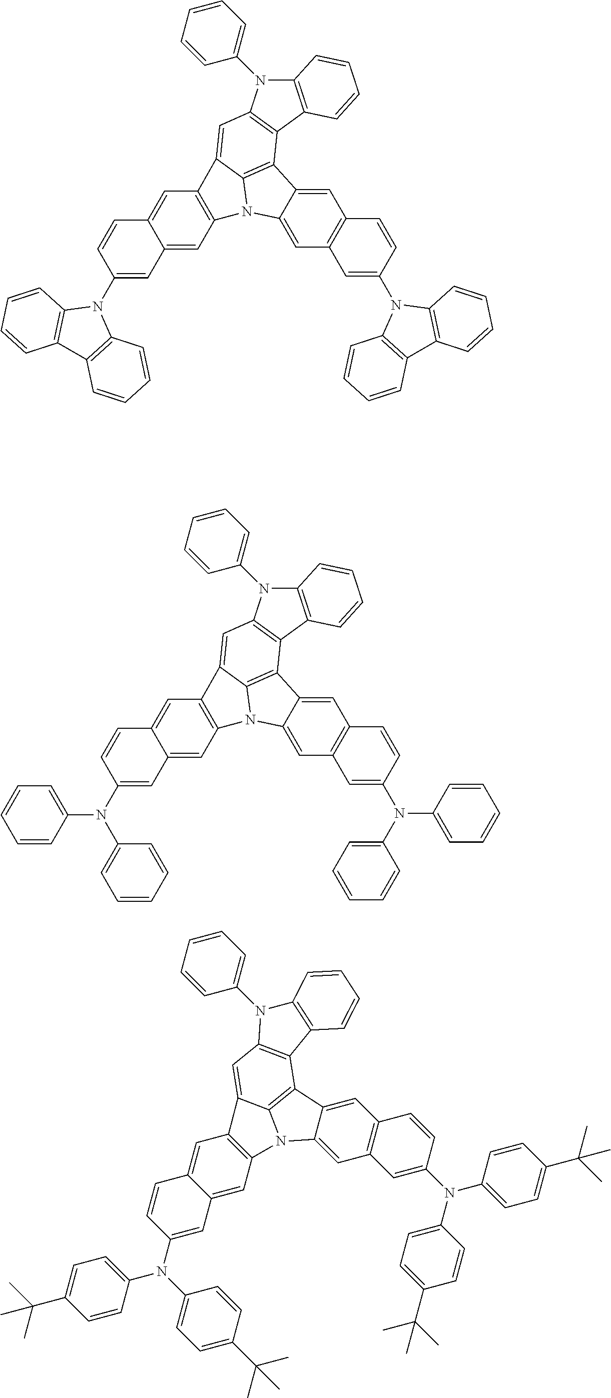 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

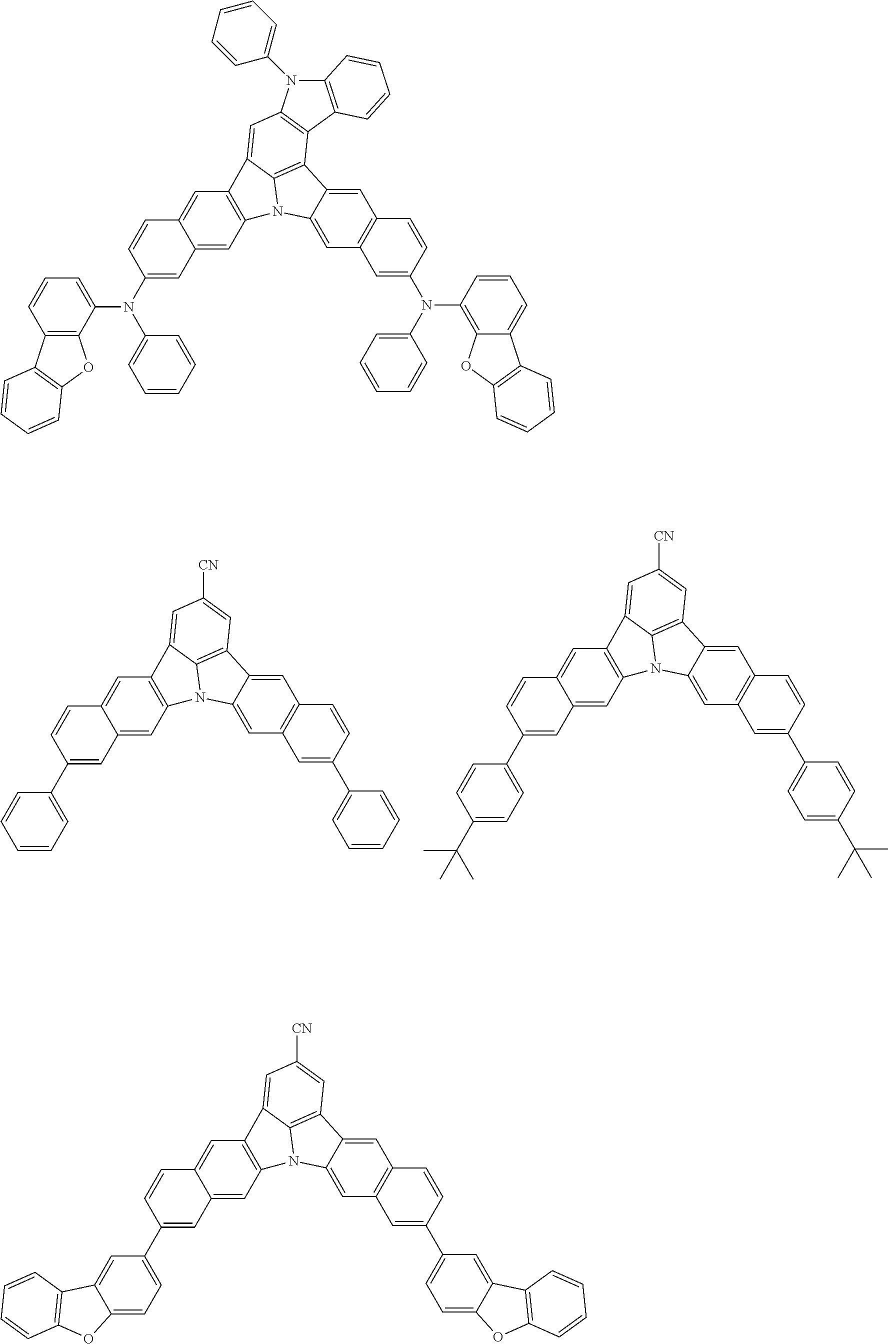

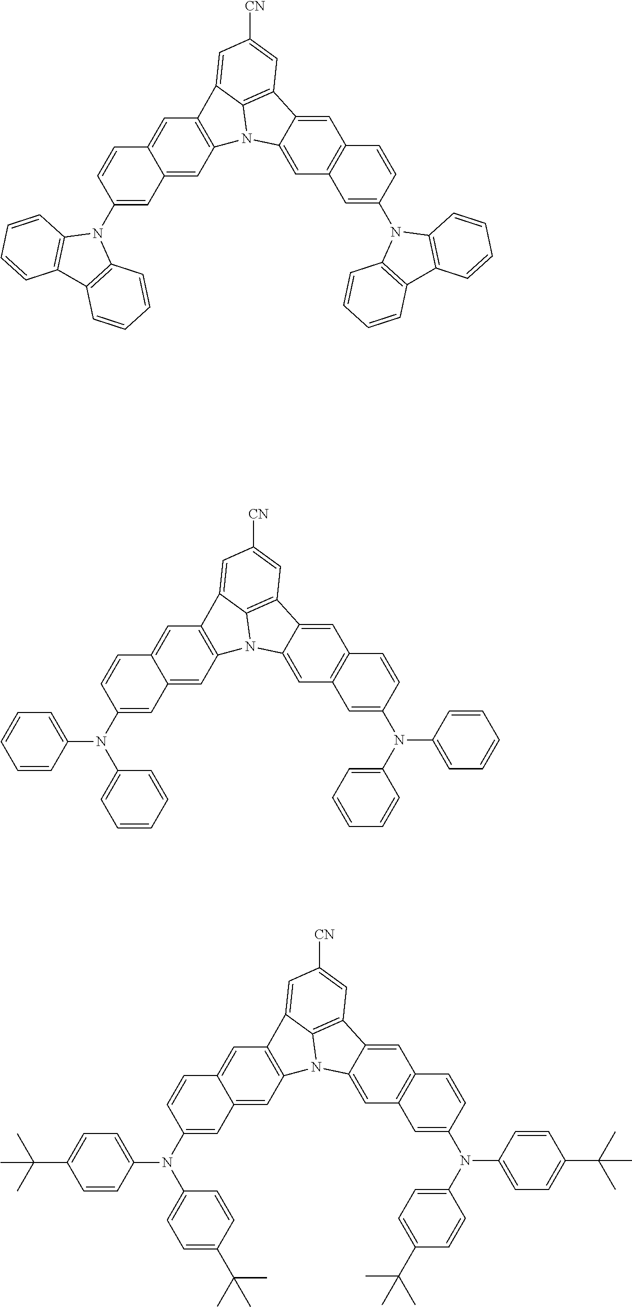 [SMILES: N#Cc1cc2c3cc4ccc(-n5c6ccccc6c6ccccc65)cc4cc3n3c4cc5cc(-n6c7ccccc7c7ccccc76)ccc5cc4c(c1)c23 ; N#Cc1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C#N)cc6c7cc8ccc(N(c9ccc(C(C)(C)C)cc9)c9ccc(C(C)(C)C)cc9)cc8cc7n(c4cc3c2)c56)cc1]

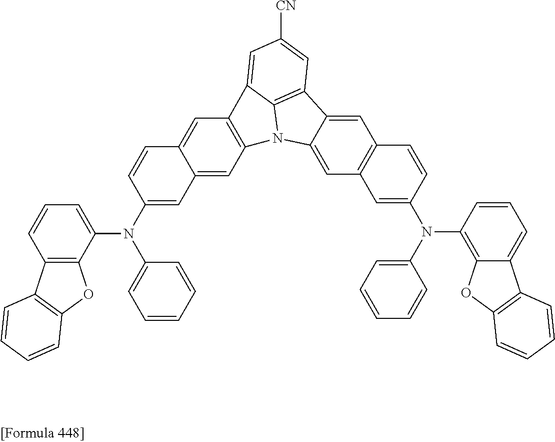 [SMILES: N#Cc1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23]

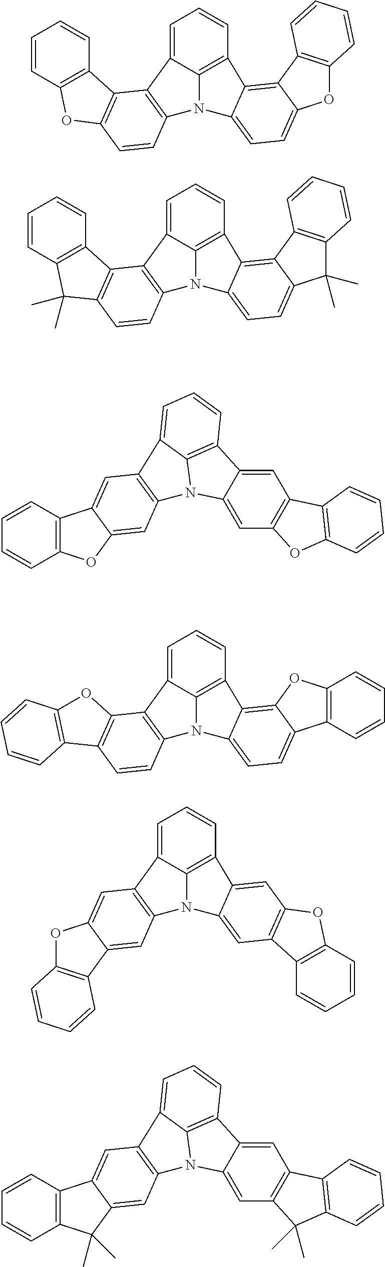

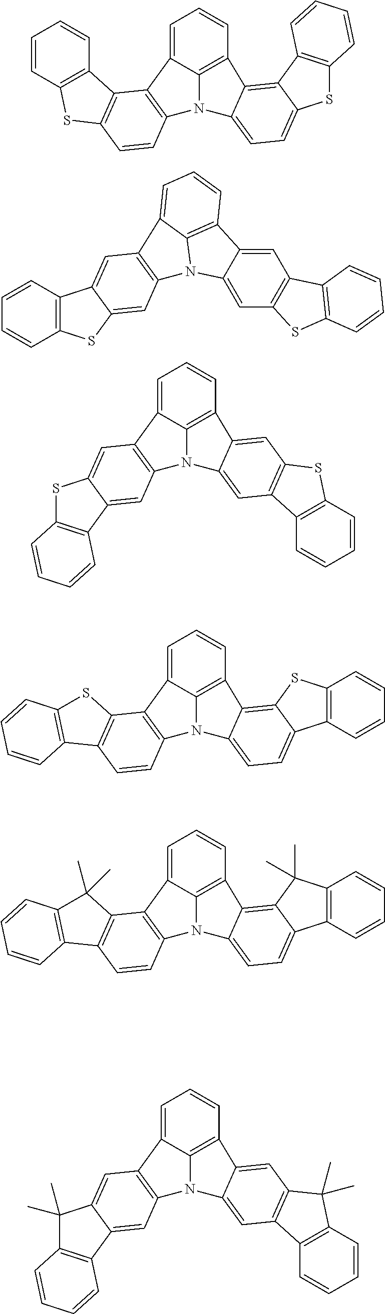

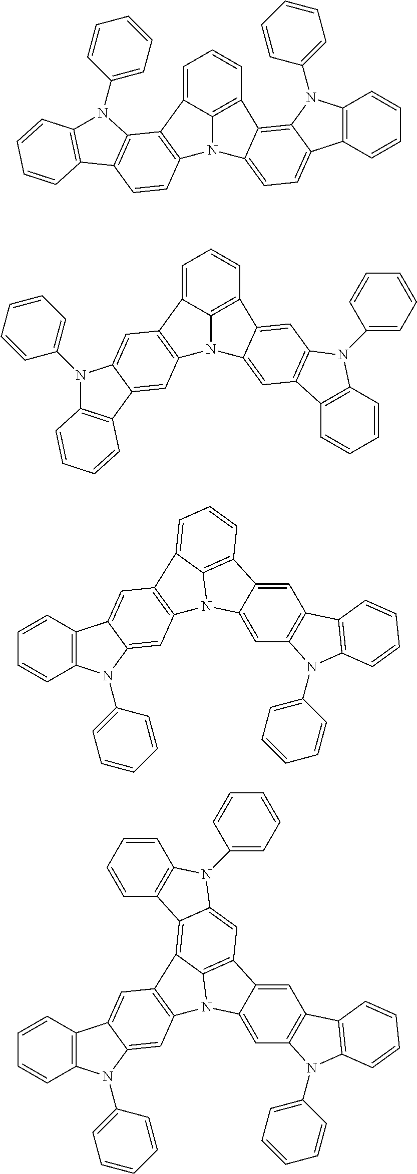

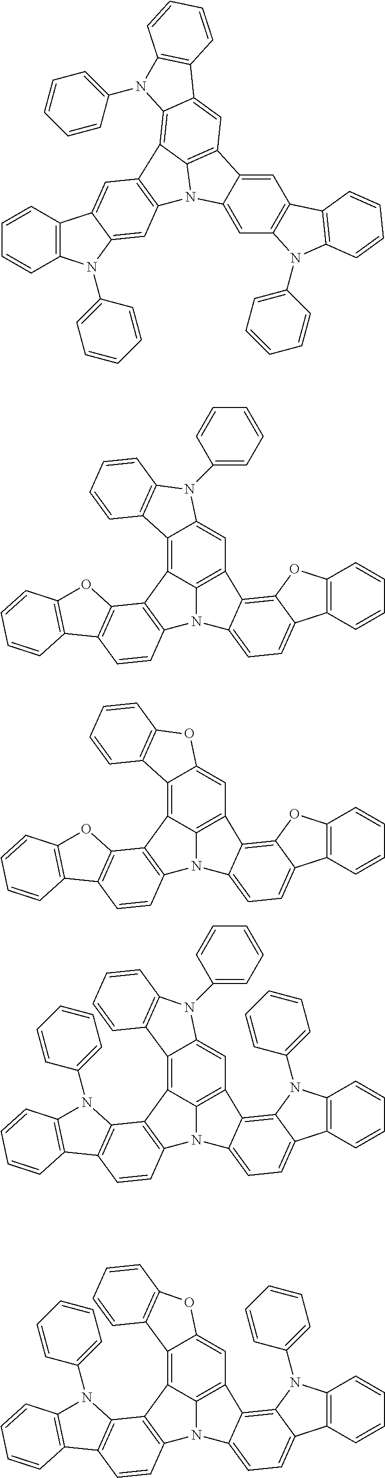

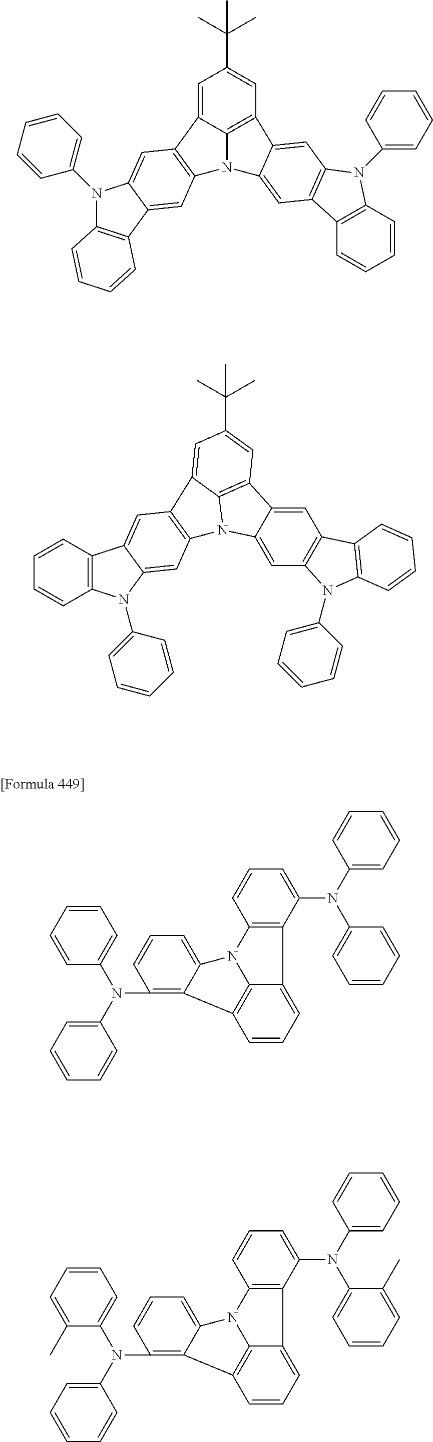 [SMILES: CC(C)(C)c1cc2c3cc4c(cc3n3c5cc6c7ccccc7n(-c7ccccc7)c6cc5c(c1)c23)c1ccccc1n4-c1ccccc1 ; CC(C)(C)c1cc2c3cc4c5ccccc5n(-c5ccccc5)c4cc3n3c4cc5c(cc4c(c1)c23)c1ccccc1n5-c1ccccc1 ; c1ccc(N(c2ccccc2)c2cccc3c2c2cccc4c5c(N(c6ccccc6)c6ccccc6)cccc5n3c24)cc1 ; Cc1ccccc1N(c1ccccc1)c1cccc2c1c1cccc3c4c(N(c5ccccc5)c5ccccc5C)cccc4n2c13]

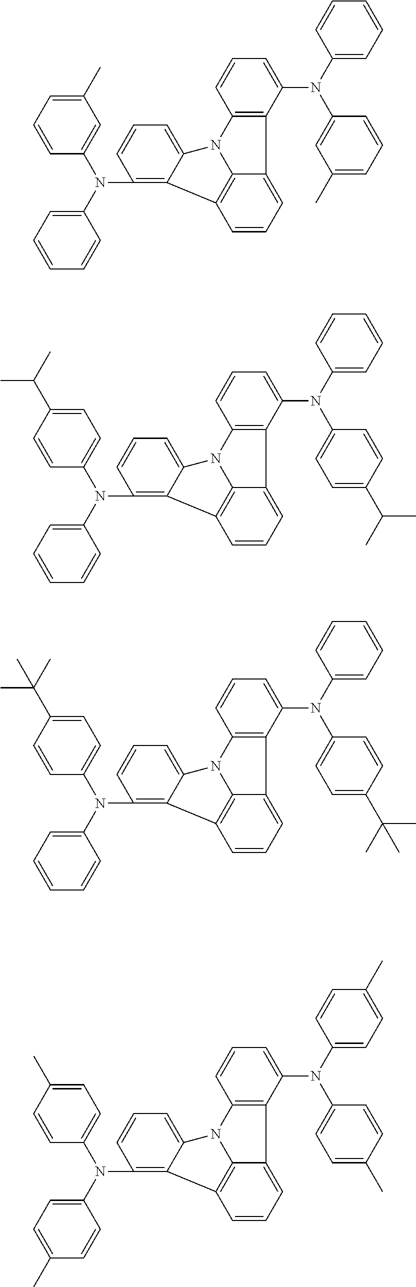

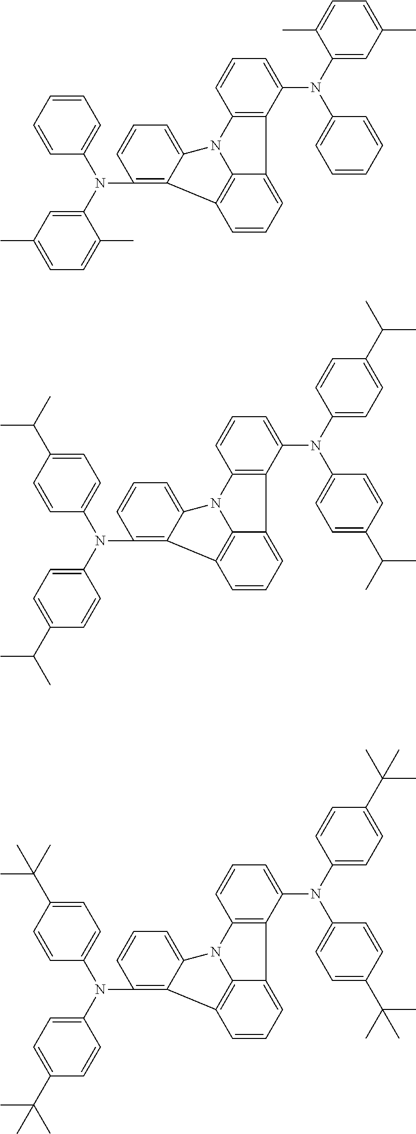

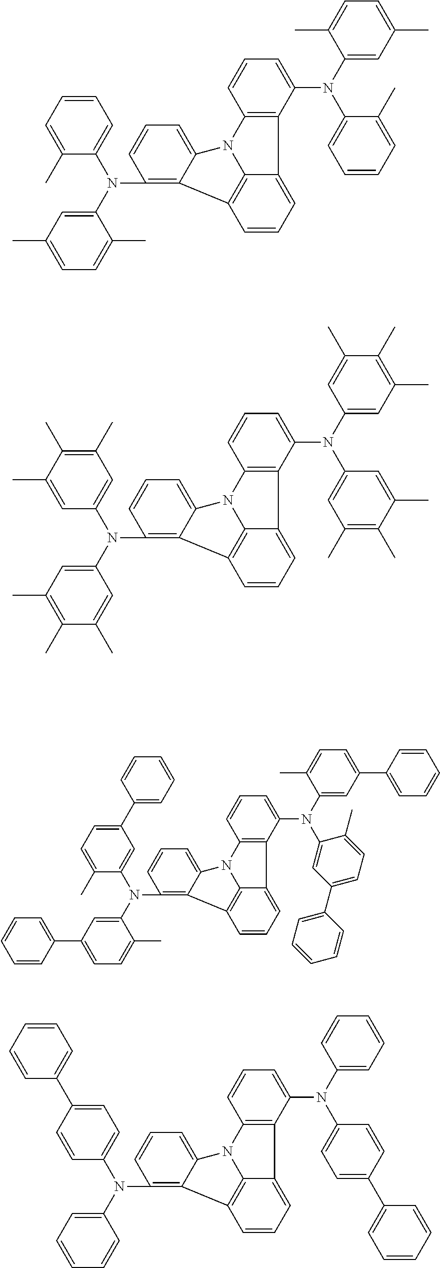

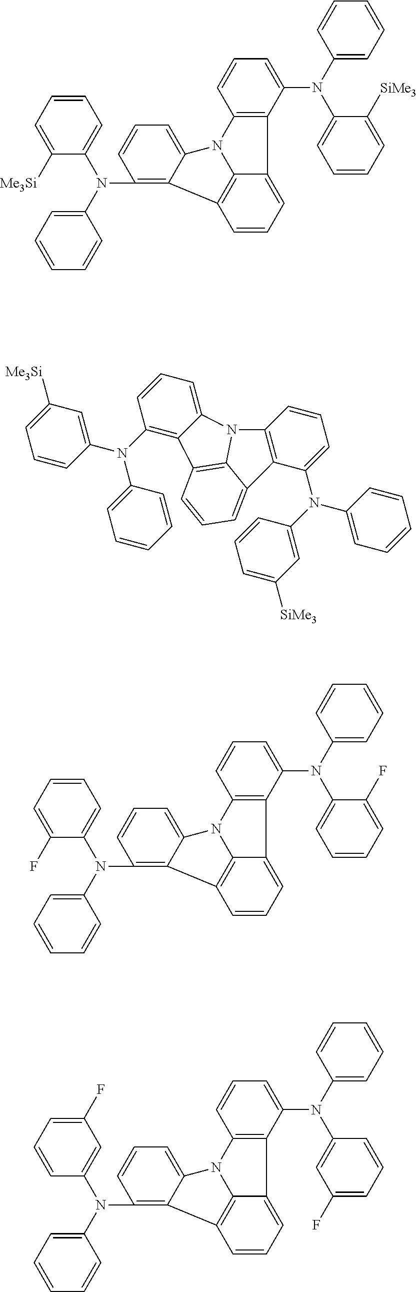 [SMILES: C[Si](C)(C)c1ccccc1N(c1ccccc1)c1cccc2c1c1cccc3c4c(N(c5ccccc5)c5ccccc5[Si](C)(C)C)cccc4n2c13 ; C[Si](C)(C)c1cccc(N(c2ccccc2)c2cccc3c2c2cccc4c5c(N(c6ccccc6)c6cccc([Si](C)(C)C)c6)cccc5n3c24)c1 ; Fc1ccccc1N(c1ccccc1)c1cccc2c1c1cccc3c4c(N(c5ccccc5)c5ccccc5F)cccc4n2c13 ; Fc1cccc(N(c2ccccc2)c2cccc3c2c2cccc4c5c(N(c6ccccc6)c6cccc(F)c6)cccc5n3c24)c1]

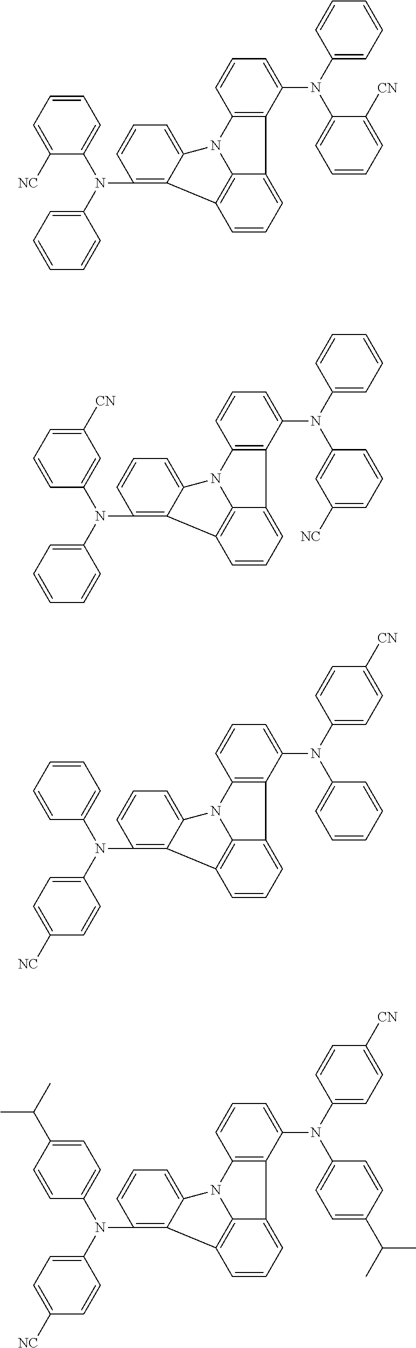 [SMILES: N#Cc1ccccc1N(c1ccccc1)c1cccc2c1c1cccc3c4c(N(c5ccccc5)c5ccccc5C#N)cccc4n2c13 ; N#Cc1cccc(N(c2ccccc2)c2cccc3c2c2cccc4c5c(N(c6ccccc6)c6cccc(C#N)c6)cccc5n3c24)c1 ; N#Cc1ccc(N(c2ccccc2)c2cccc3c2c2cccc4c5c(N(c6ccccc6)c6ccc(C#N)cc6)cccc5n3c24)cc1 ; CC(C)c1ccc(N(c2ccc(C#N)cc2)c2cccc3c2c2cccc4c5c(N(c6ccc(C#N)cc6)c6ccc(C(C)C)cc6)cccc5n3c24)cc1]

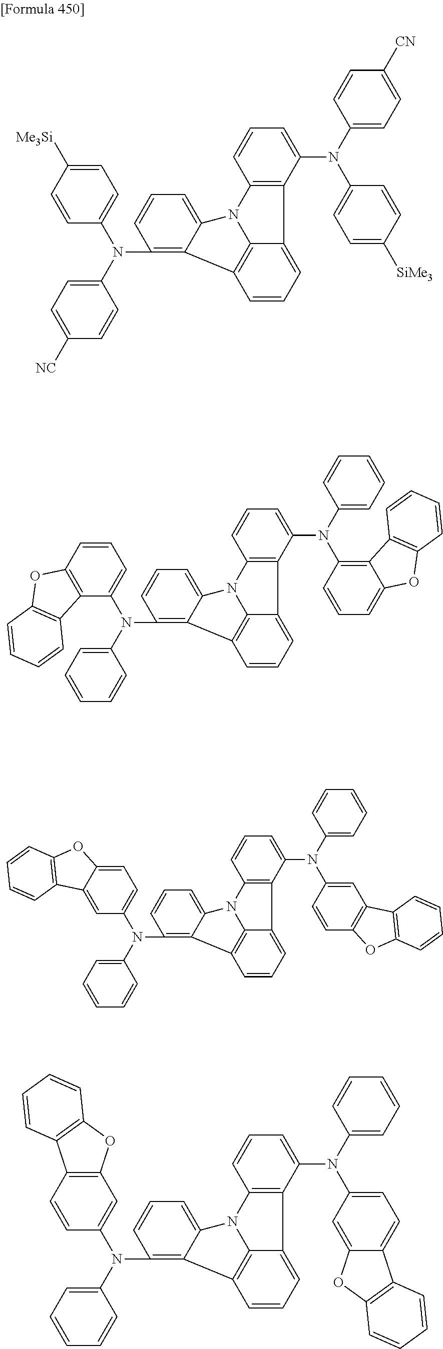 [SMILES: C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1 ; c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1 ; c1ccc(N(c2ccc3c(c2)oc2ccccc23)c2cccc3c2c2cccc4c5c(N(c6ccccc6)c6ccc7c(c6)oc6ccccc67)cccc5n3c24)cc1]

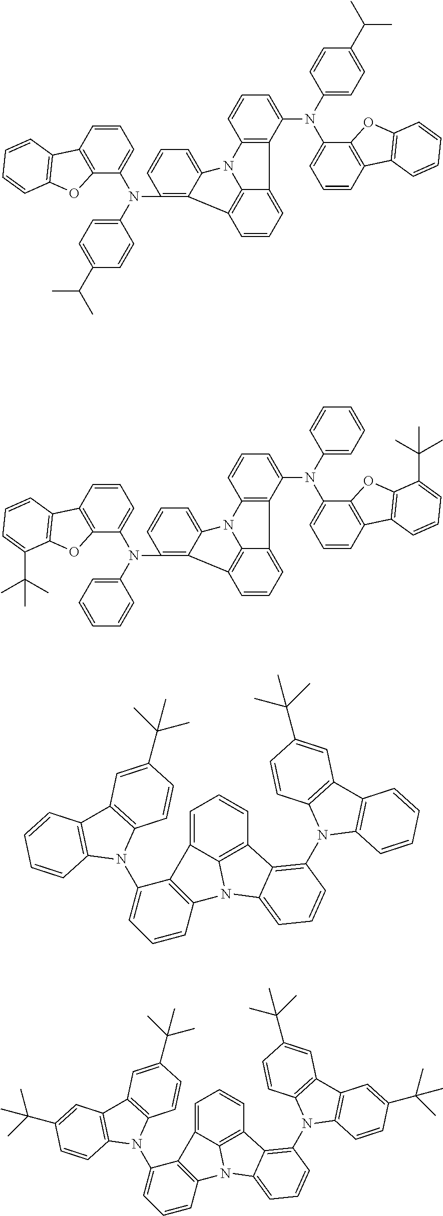

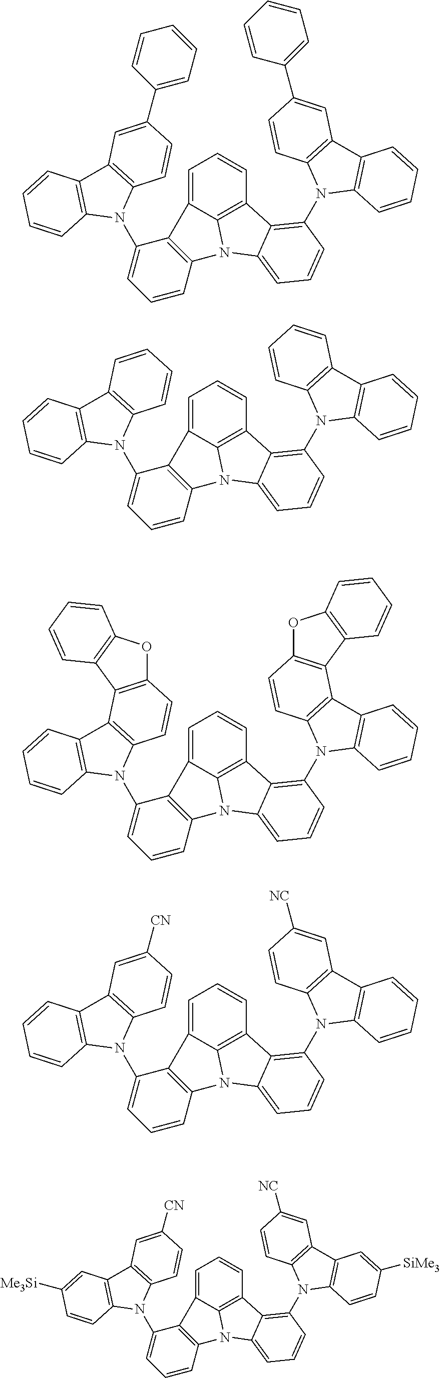

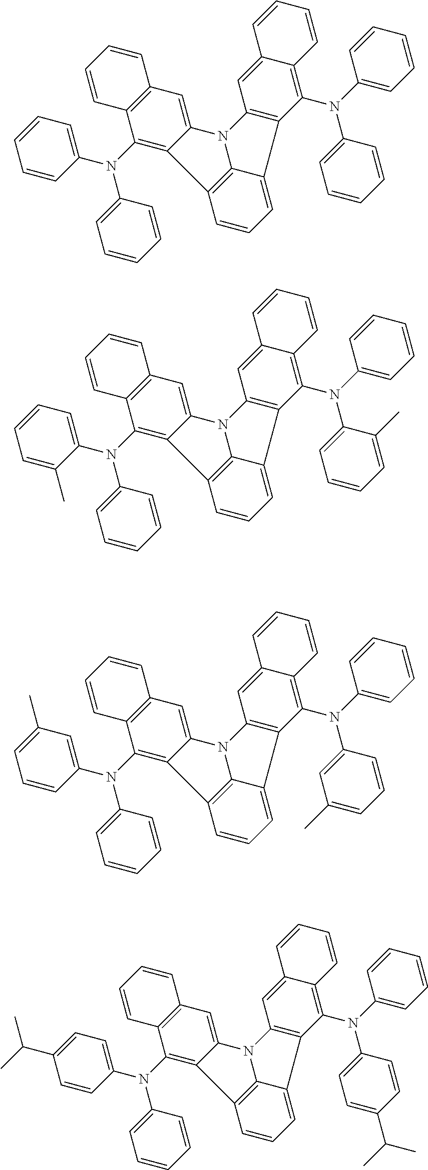 [SMILES: c1ccc(N(c2ccccc2)c2c3ccccc3cc3c2c2cccc4c5c(N(c6ccccc6)c6ccccc6)c6ccccc6cc5n3c24)cc1 ; Cc1ccccc1N(c1ccccc1)c1c2ccccc2cc2c1c1cccc3c4c(N(c5ccccc5)c5ccccc5C)c5ccccc5cc4n2c13 ; Cc1cccc(N(c2ccccc2)c2c3ccccc3cc3c2c2cccc4c5c(N(c6ccccc6)c6cccc(C)c6)c6ccccc6cc5n3c24)c1 ; CC(C)c1ccc(N(c2ccccc2)c2c3ccccc3cc3c2c2cccc4c5c(N(c6ccccc6)c6ccc(C(C)C)cc6)c6ccccc6cc5n3c24)cc1]

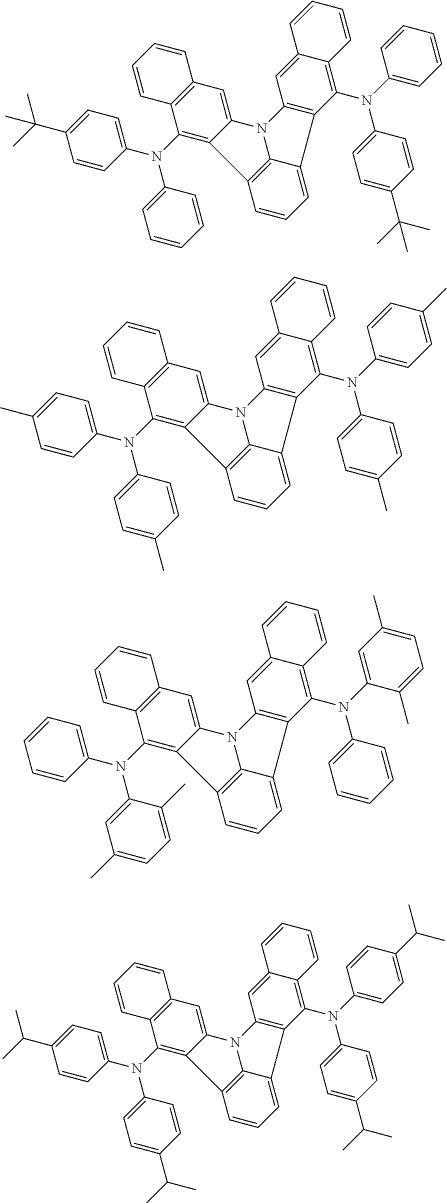

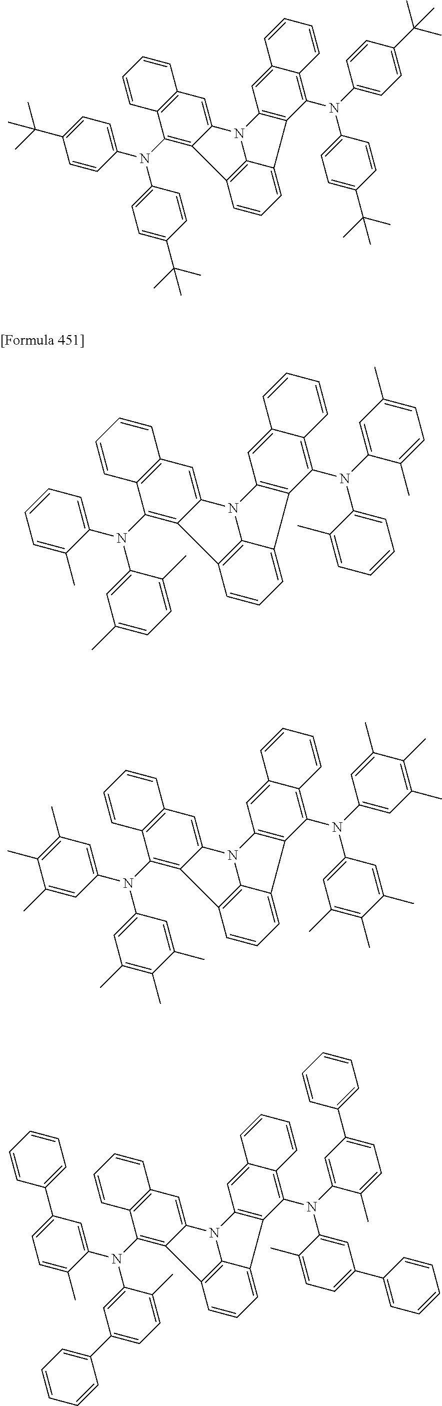

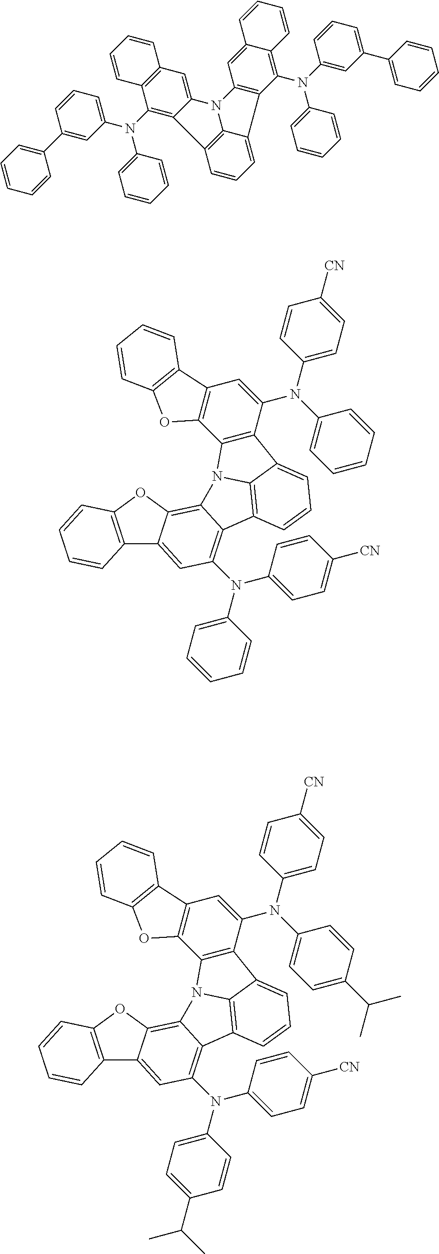 [SMILES: c1ccc(-c2cccc(N(c3ccccc3)c3c4ccccc4cc4c3c3cccc5c6c(N(c7ccccc7)c7cccc(-c8ccccc8)c7)c7ccccc7cc6n4c35)c2)cc1 ; N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1]

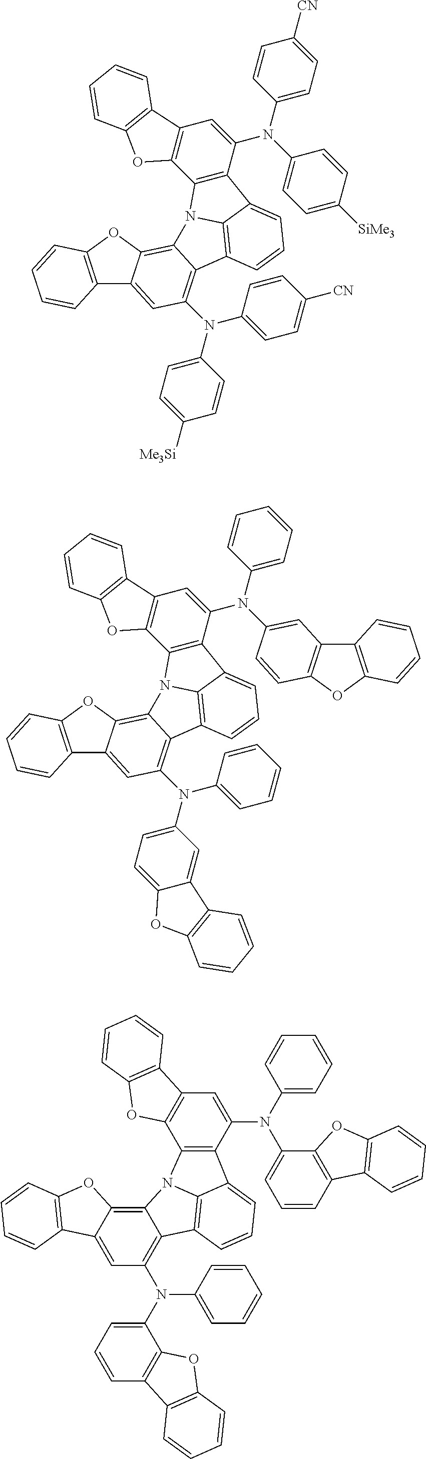 [SMILES: C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1 ; c1ccc(N(c2ccc3oc4ccccc4c3c2)c2cc3c4ccccc4oc3c3c2c2cccc4c5c(N(c6ccccc6)c6ccc7oc8ccccc8c7c6)cc6c7ccccc7oc6c5n3c24)cc1 ; c1ccc(N(c2cccc3c2oc2ccccc23)c2cc3c4ccccc4oc3c3c2c2cccc4c5c(N(c6ccccc6)c6cccc7c6oc6ccccc67)cc6c7ccccc7oc6c5n3c24)cc1]

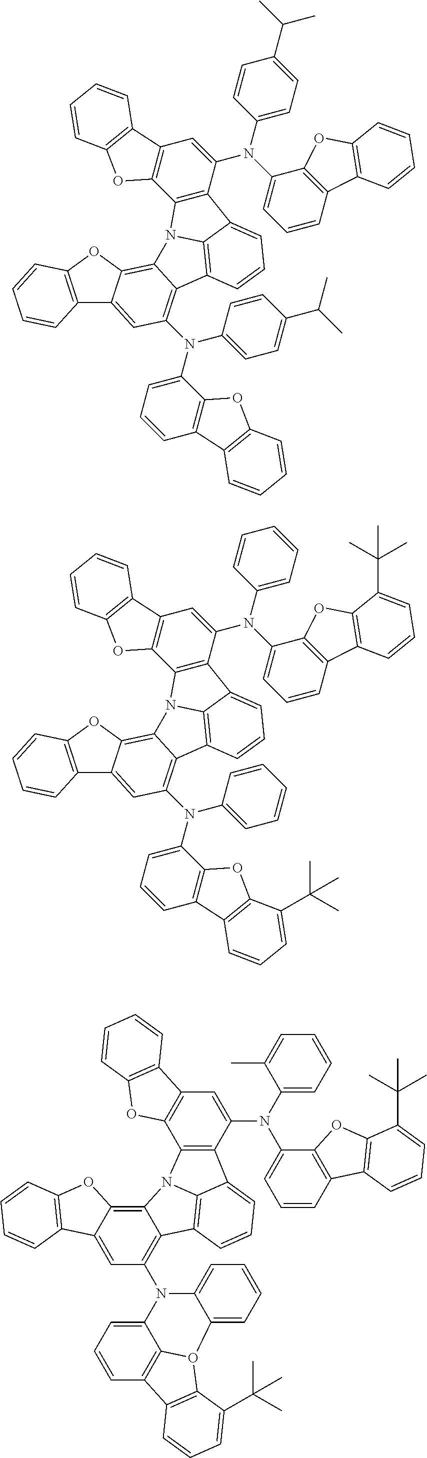

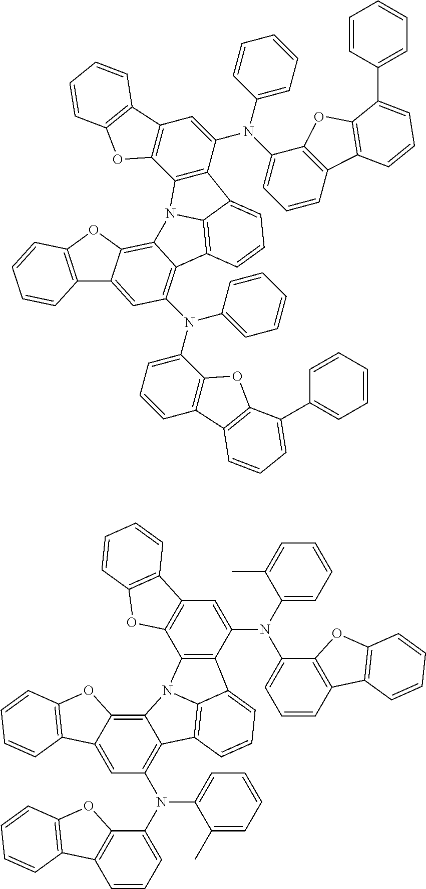 [SMILES: c1ccc(-c2cccc3c2oc2c(N(c4ccccc4)c4cc5c6ccccc6oc5c5c4c4cccc6c7c(N(c8ccccc8)c8cccc9c8oc8c(-c%10ccccc%10)cccc89)cc8c9ccccc9oc8c7n5c46)cccc23)cc1 ; Cc1ccccc1N(c1cccc2c1oc1ccccc12)c1cc2c3ccccc3oc2c2c1c1cccc3c4c(N(c5ccccc5C)c5cccc6c5oc5ccccc56)cc5c6ccccc6oc5c4n2c13]

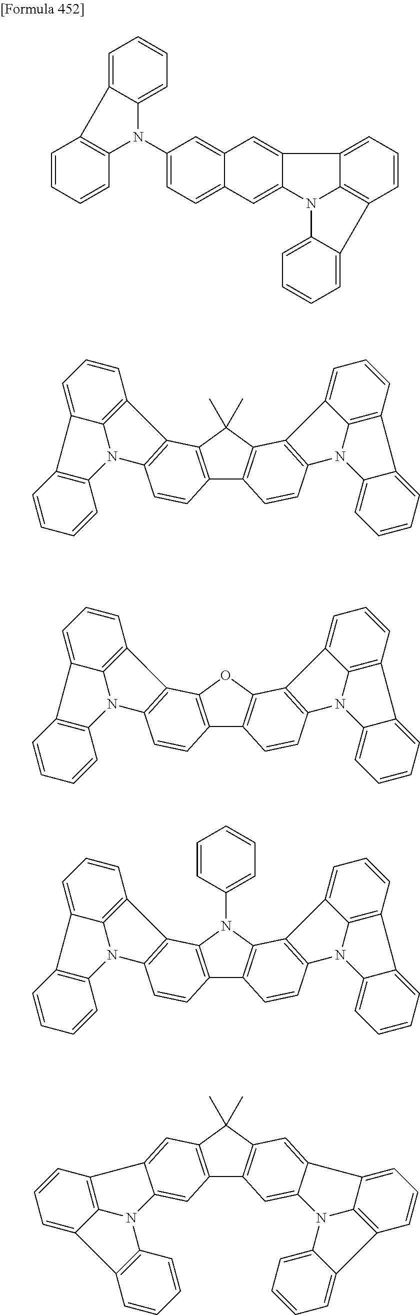 [SMILES: c1ccc2c(c1)c1ccccc1n2-c1ccc2cc3c(cc2c1)c1cccc2c4ccccc4n3c21 ; CC1(C)c2c(ccc3c2c2cccc4c5ccccc5n3c42)-c2ccc3c(c21)c1cccc2c4ccccc4n3c21 ; c1ccc2c(c1)c1cccc3c4c5oc6c(ccc7c6c6cccc8c9ccccc9n7c86)c5ccc4n2c13 ; c1ccc(-n2c3c(ccc4c3c3cccc5c6ccccc6n4c53)c3ccc4c(c5cccc6c7ccccc7n4c65)c32)cc1 ; CC1(C)c2cc3c4cccc5c6ccccc6n(c3cc2-c2cc3c(cc21)c1cccc2c6ccccc6n3c21)c54]

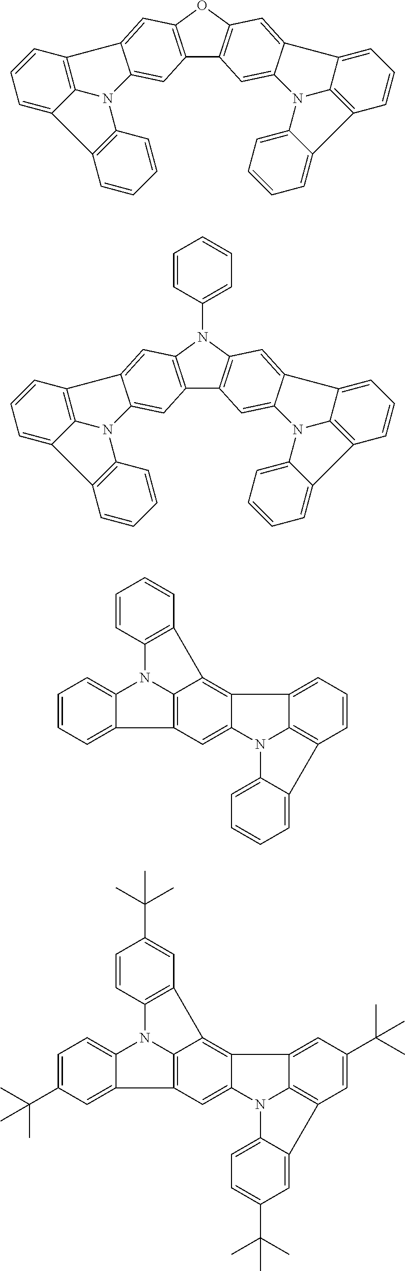 [SMILES: c1ccc2c(c1)c1cccc3c4cc5oc6cc7c8cccc9c%10ccccc%10n(c7cc6c5cc4n2c13)c98 ; c1ccc(-n2c3cc4c5cccc6c7ccccc7n(c4cc3c3cc4c(cc32)c2cccc3c7ccccc7n4c32)c65)cc1 ; c1ccc2c(c1)c1cccc3c4c5c6ccccc6n6c7ccccc7c(cc4n2c13)c56 ; CC(C)(C)c1ccc2c(c1)c1cc(C(C)(C)C)cc3c4c5c6cc(C(C)(C)C)ccc6n6c7ccc(C(C)(C)C)cc7c(cc4n2c13)c56]

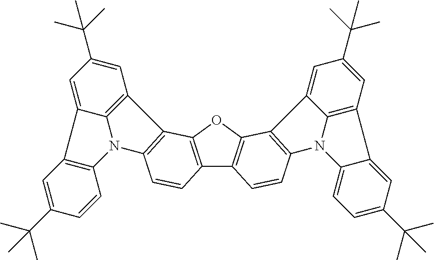 [SMILES: CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

### Compound Represented by Formula (5)

The compound represented by the formula (5) will be described below. The compound represented by the formula (5) corresponds to a compound represented by the formula (41-3).

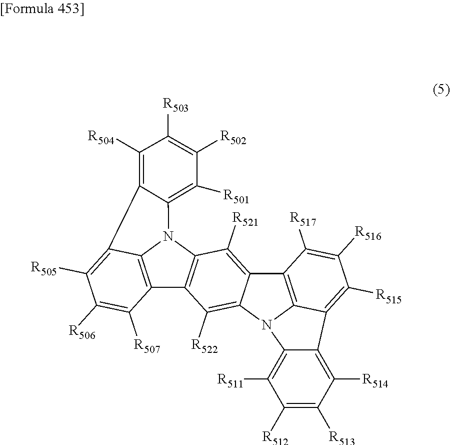

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;
R₅₀₁ to R₅₀₇ and R₅₁₁ to R₅₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ or R₅₁₁ to R₅₁₇ are each a group represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52) below.

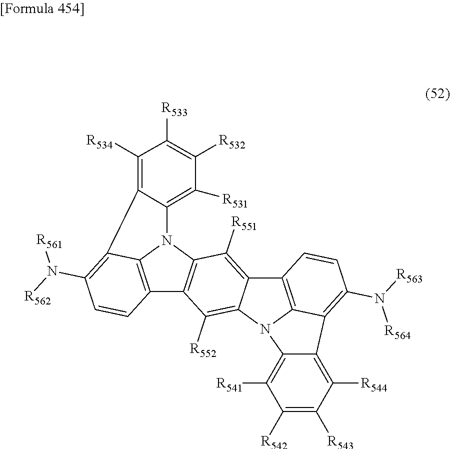

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄, R₅₄₁ to R₅₄₄, R₅₅₁, and R₅₅₂ forming neither the substituted or unsubstituted monocyclic ring nor the substituted or unsubstituted fused ring are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53) below.

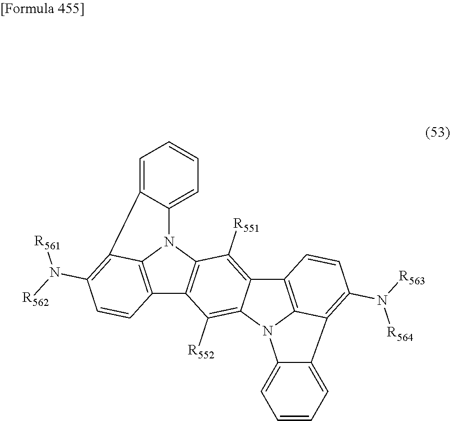

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are each a hydrogen atom.

In an exemplary arrangement, a substituent for a “substituted or unsubstituted” group in the formulae (5), (52), and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring carbon atoms.

### Specific Examples of Compound Represented by Formula (5)

Specific examples of the compound represented by the formula (5) include compounds as below.

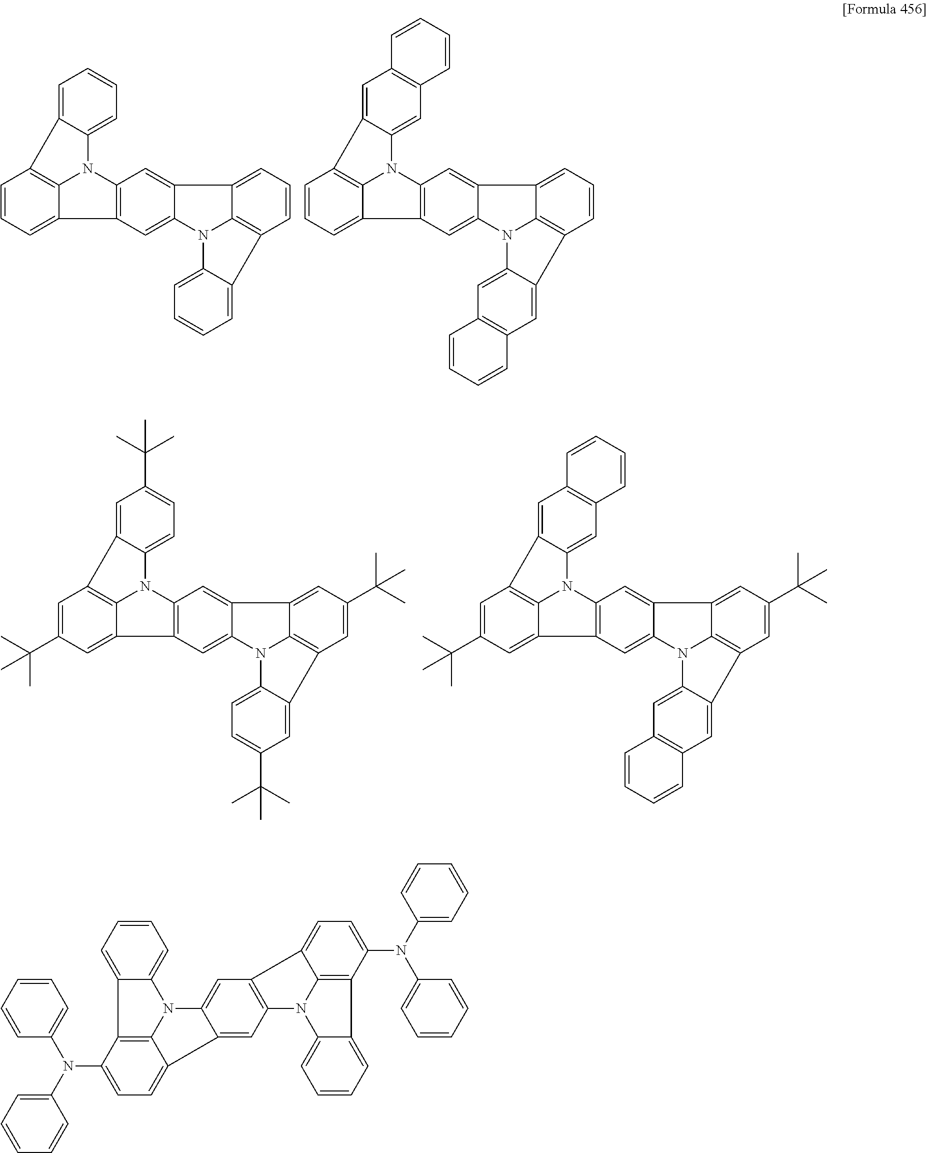

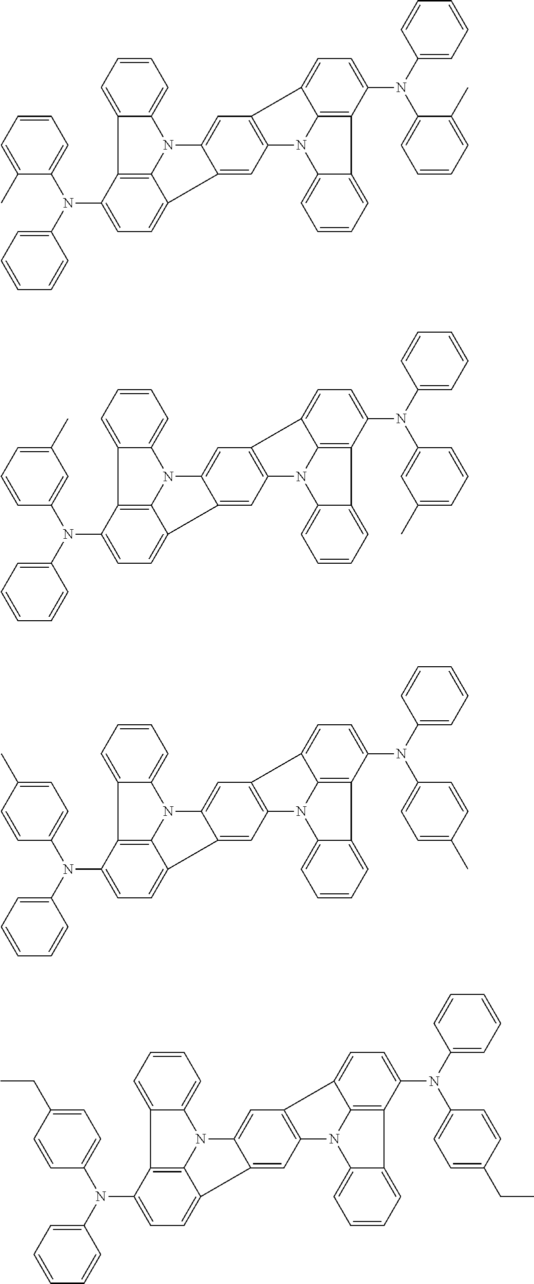

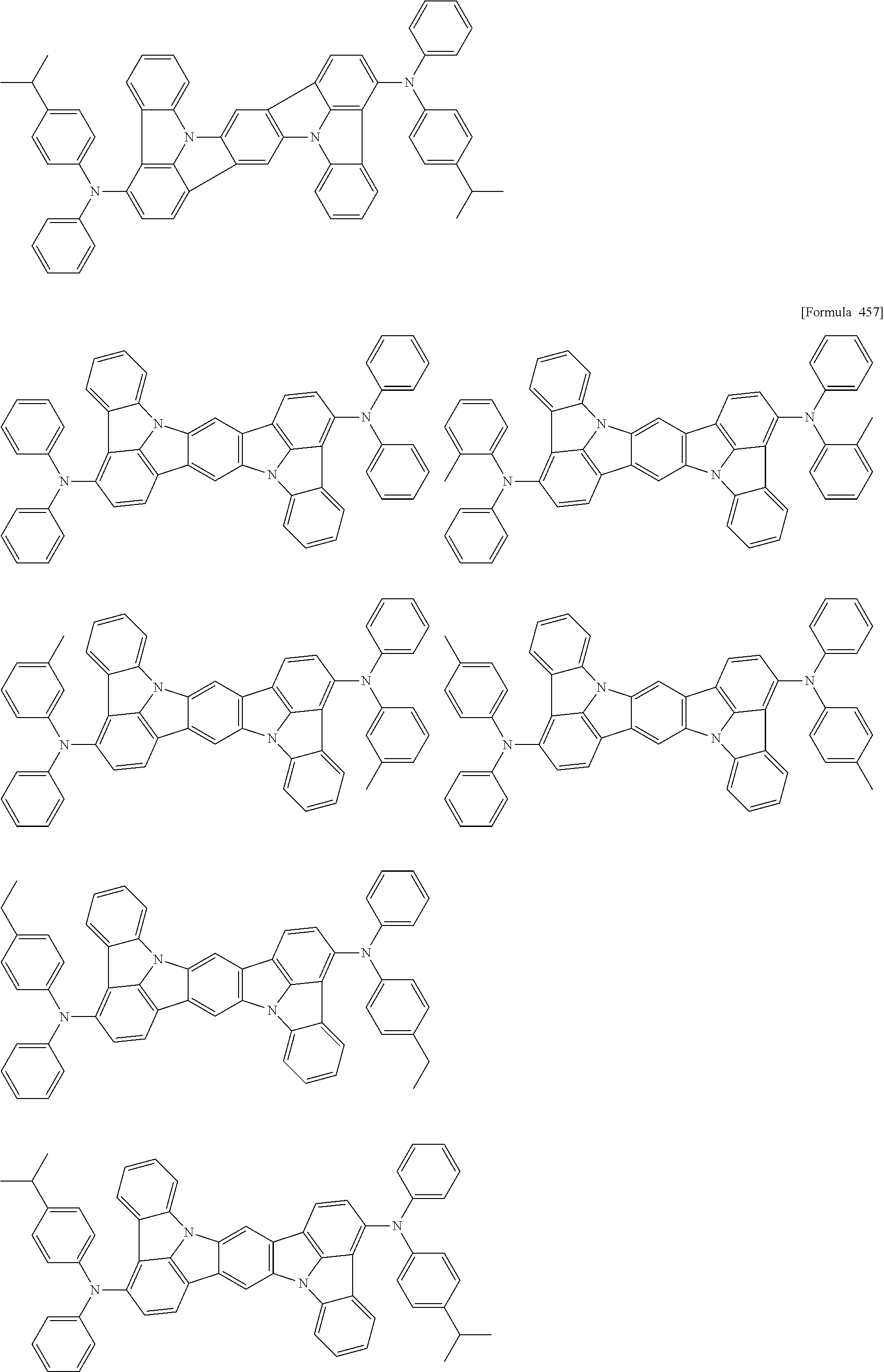

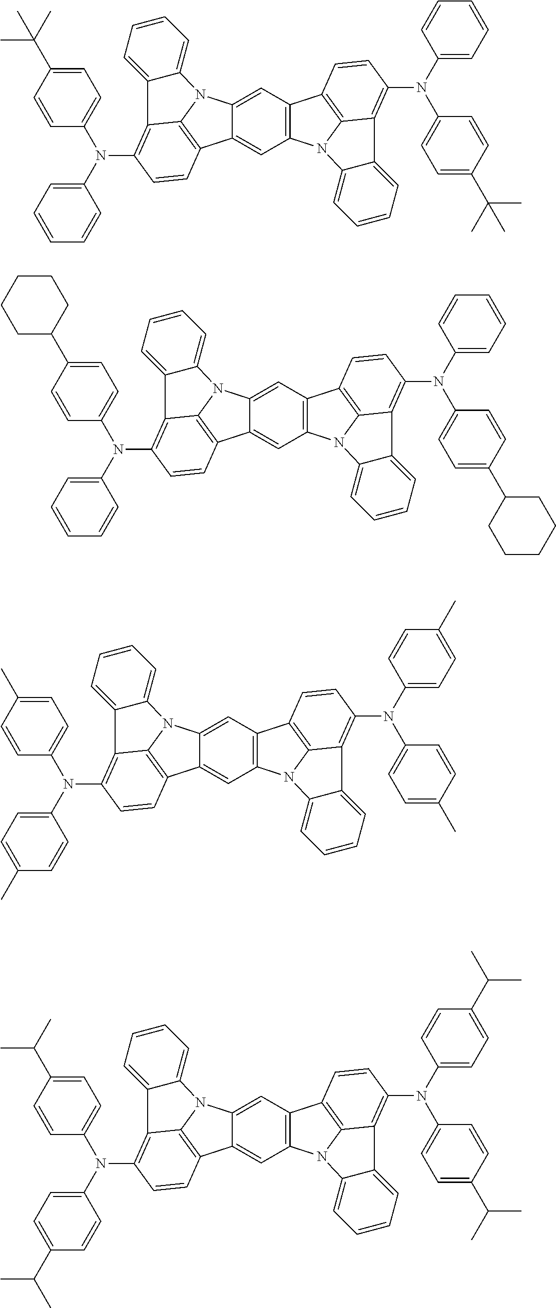

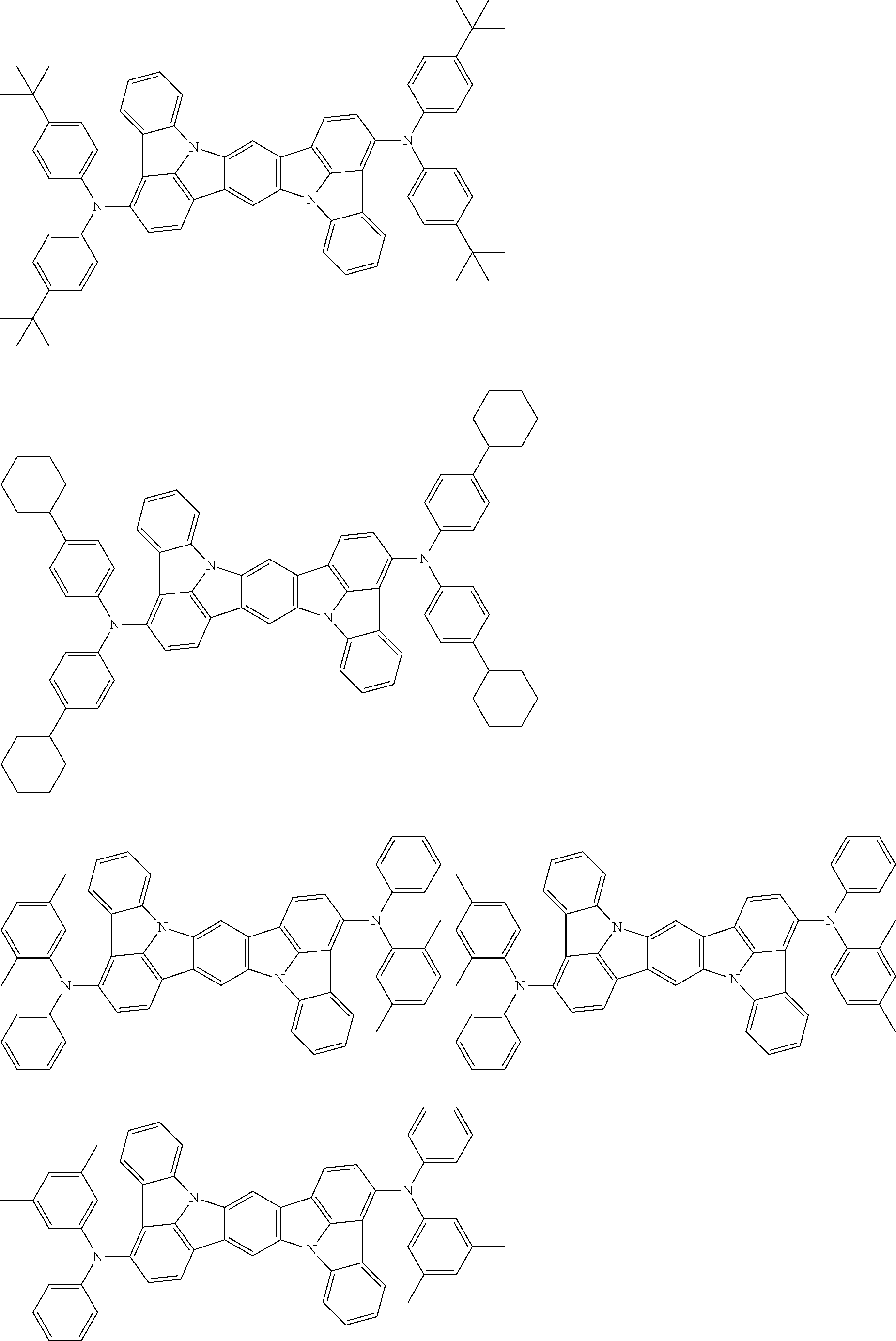

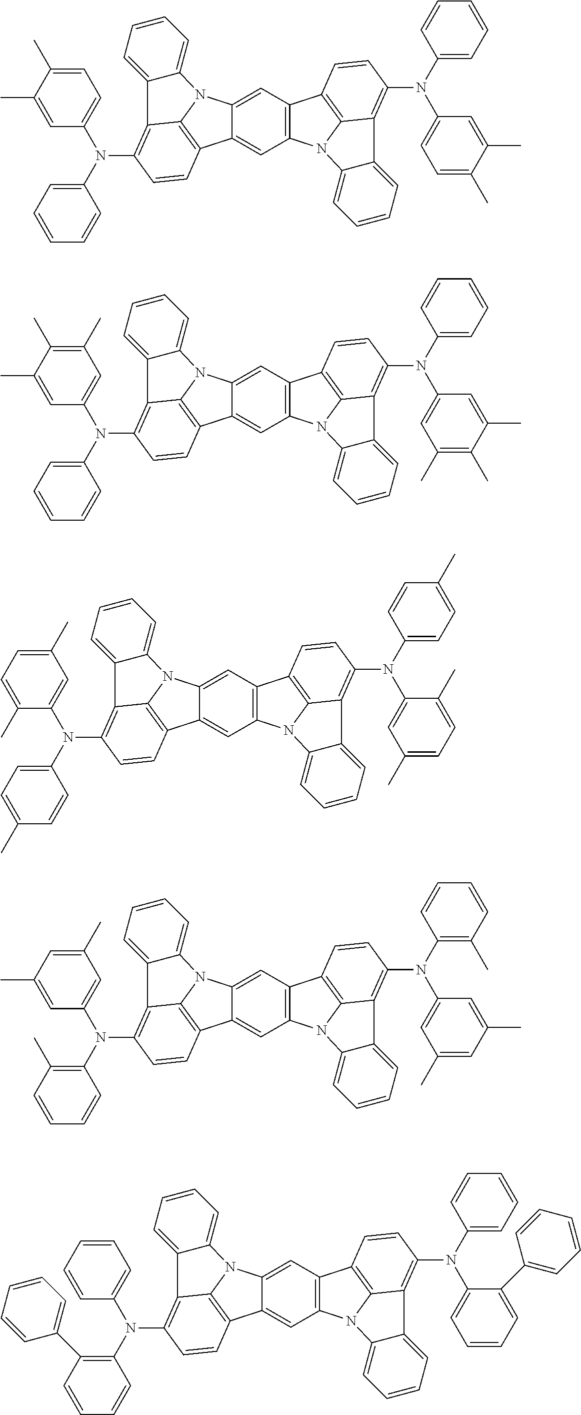

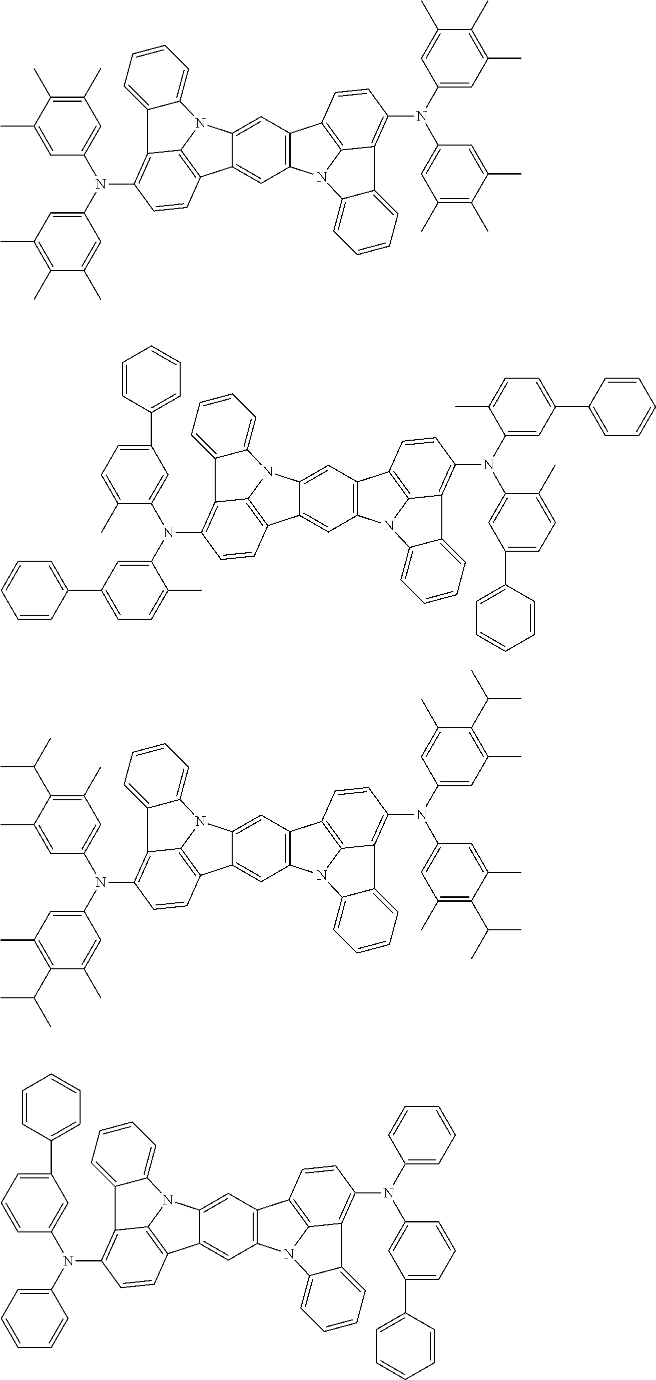

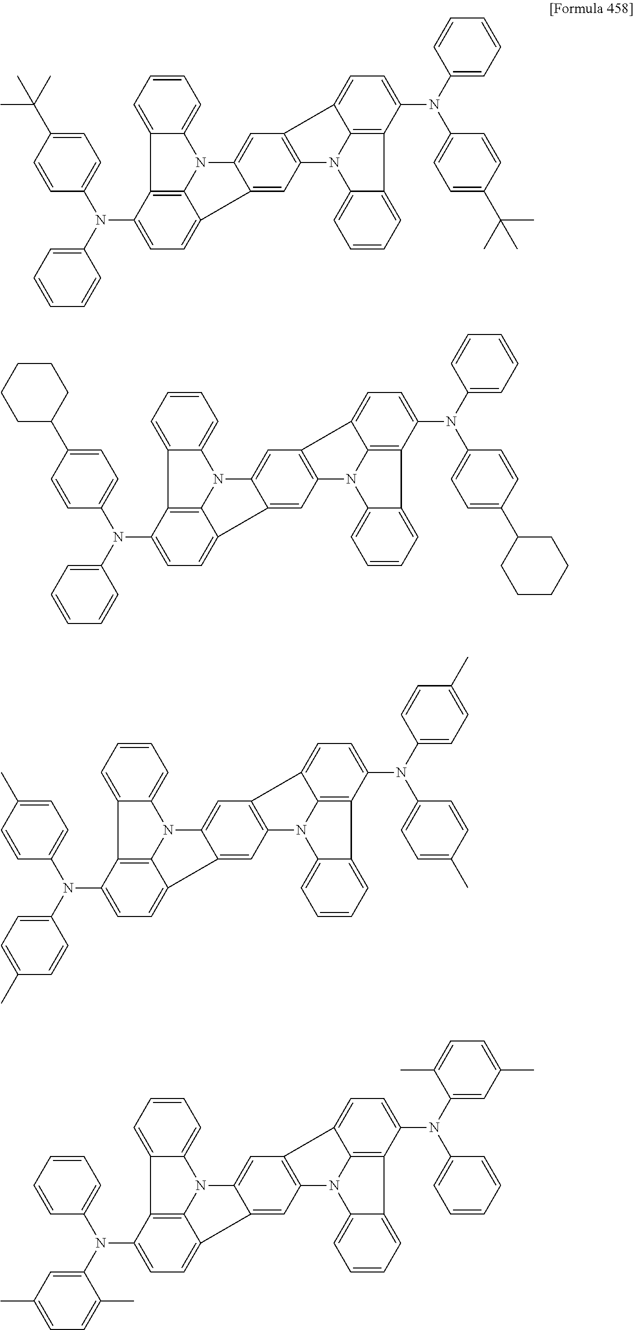

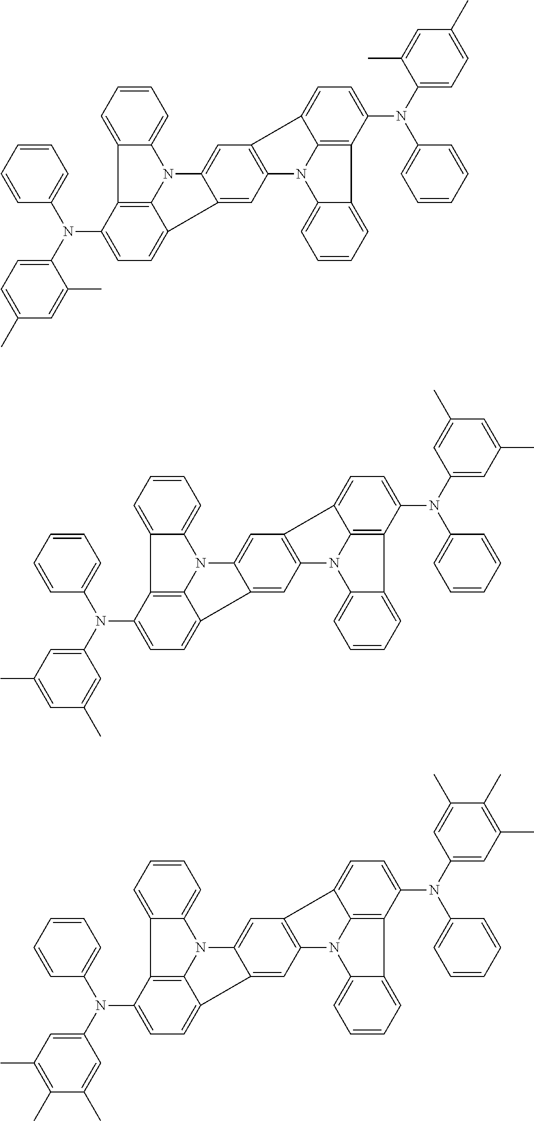

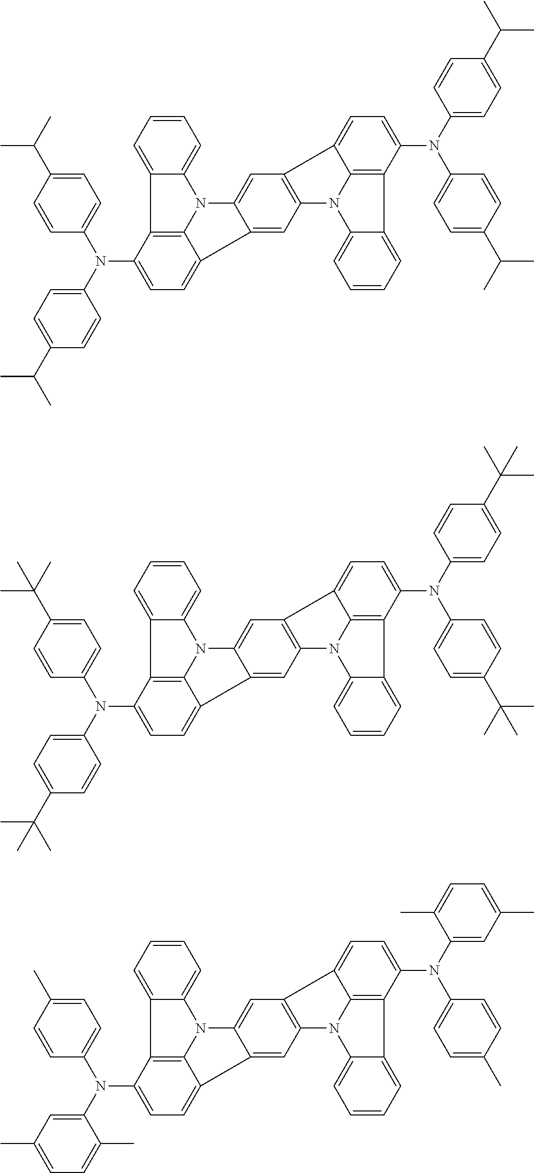

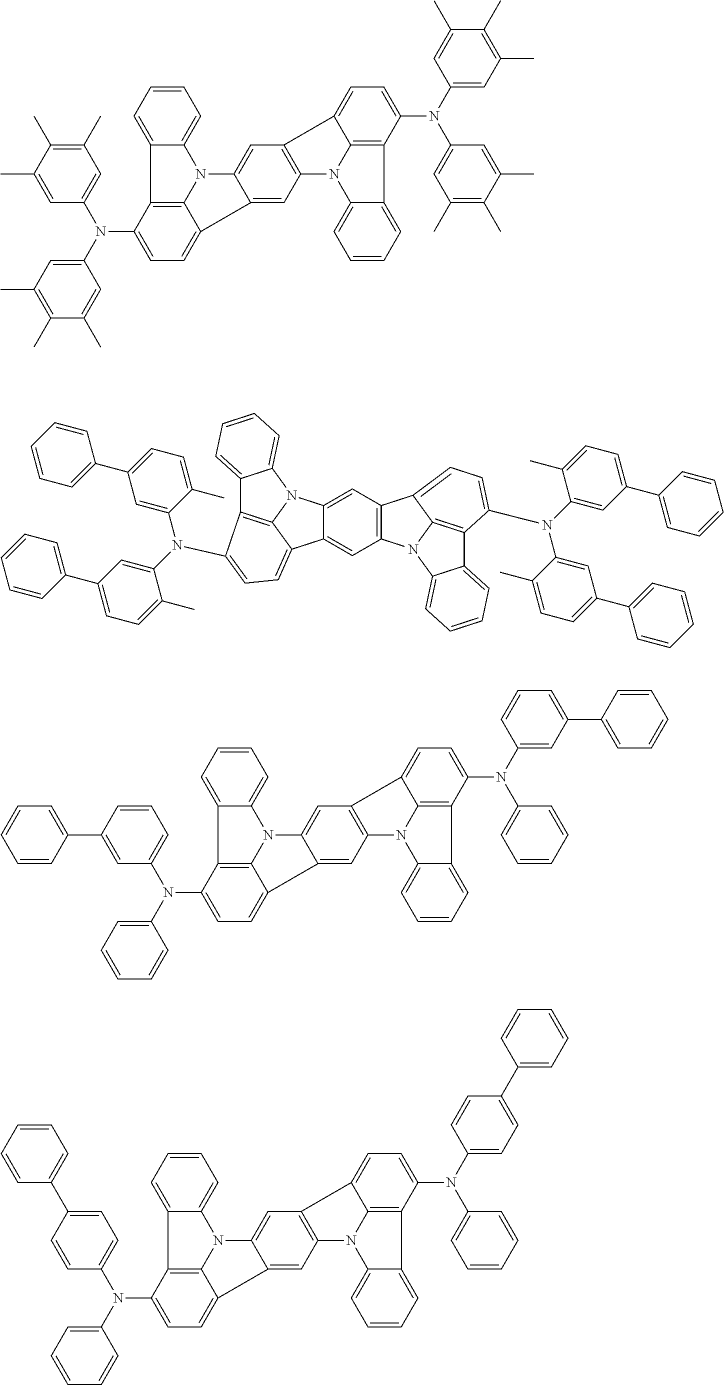

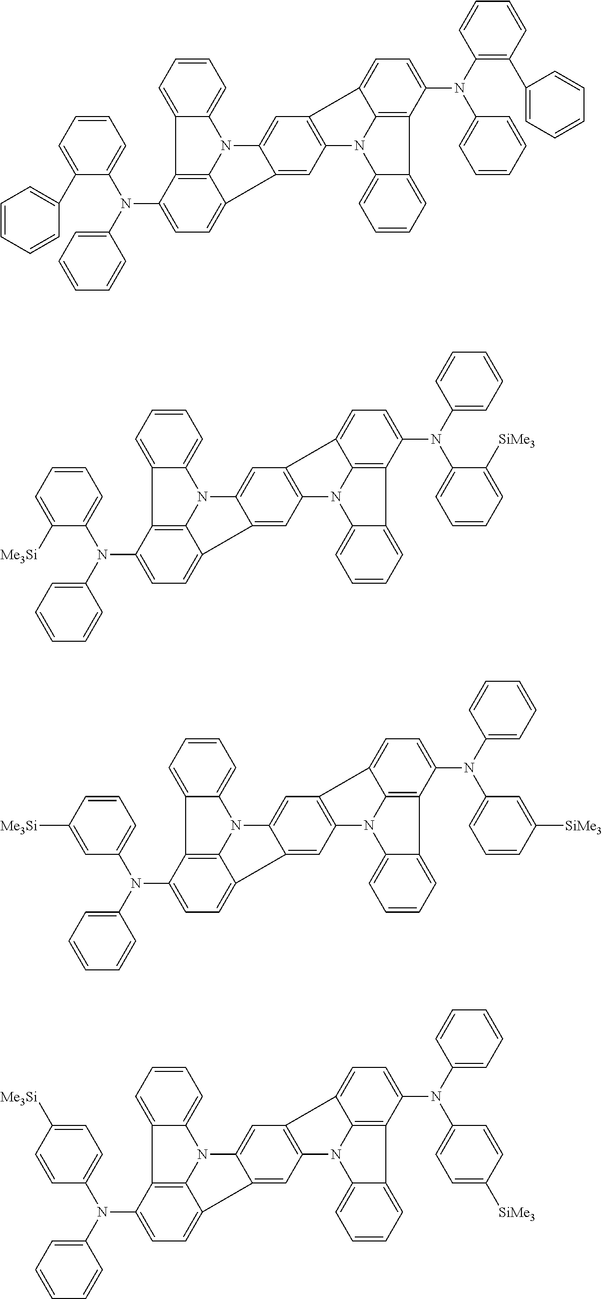 [SMILES: c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1 ; C[Si](C)(C)c1ccc(N(c2ccccc2)c2ccc3c4cc5c(cc4n4c6ccccc6c2c34)c2ccc(N(c3ccccc3)c3ccc([Si](C)(C)C)cc3)c3c4ccccc4n5c23)cc1]

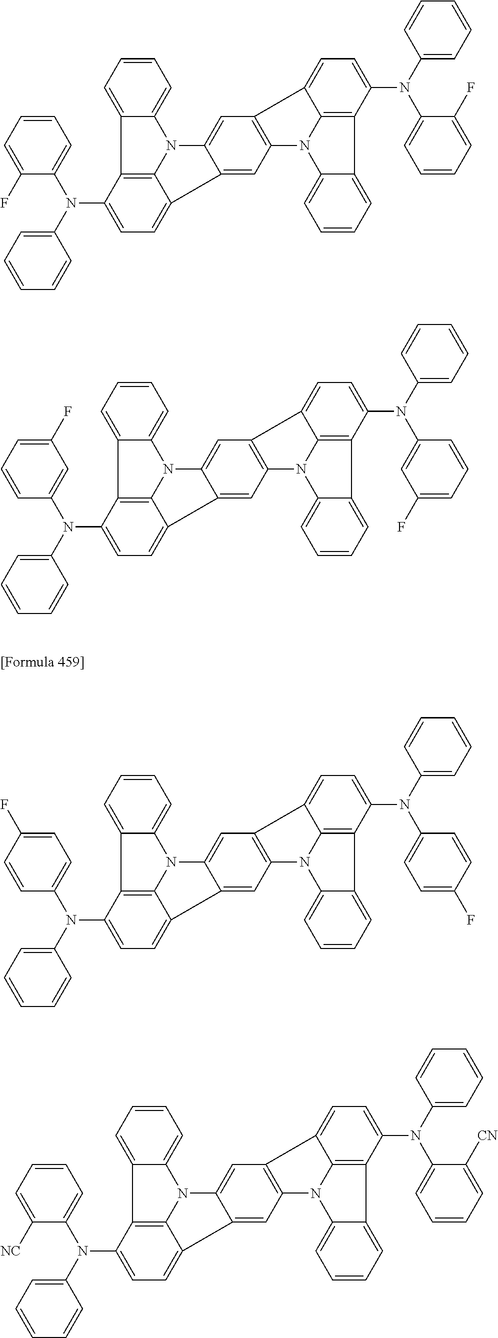 [SMILES: Fc1ccccc1N(c1ccccc1)c1ccc2c3cc4c(cc3n3c5ccccc5c1c23)c1ccc(N(c2ccccc2)c2ccccc2F)c2c3ccccc3n4c12 ; Fc1cccc(N(c2ccccc2)c2ccc3c4cc5c(cc4n4c6ccccc6c2c34)c2ccc(N(c3ccccc3)c3cccc(F)c3)c3c4ccccc4n5c23)c1 ; Fc1ccc(N(c2ccccc2)c2ccc3c4cc5c(cc4n4c6ccccc6c2c34)c2ccc(N(c3ccccc3)c3ccc(F)cc3)c3c4ccccc4n5c23)cc1 ; N#Cc1ccccc1N(c1ccccc1)c1ccc2c3cc4c(cc3n3c5ccccc5c1c23)c1ccc(N(c2ccccc2)c2ccccc2C#N)c2c3ccccc3n4c12]

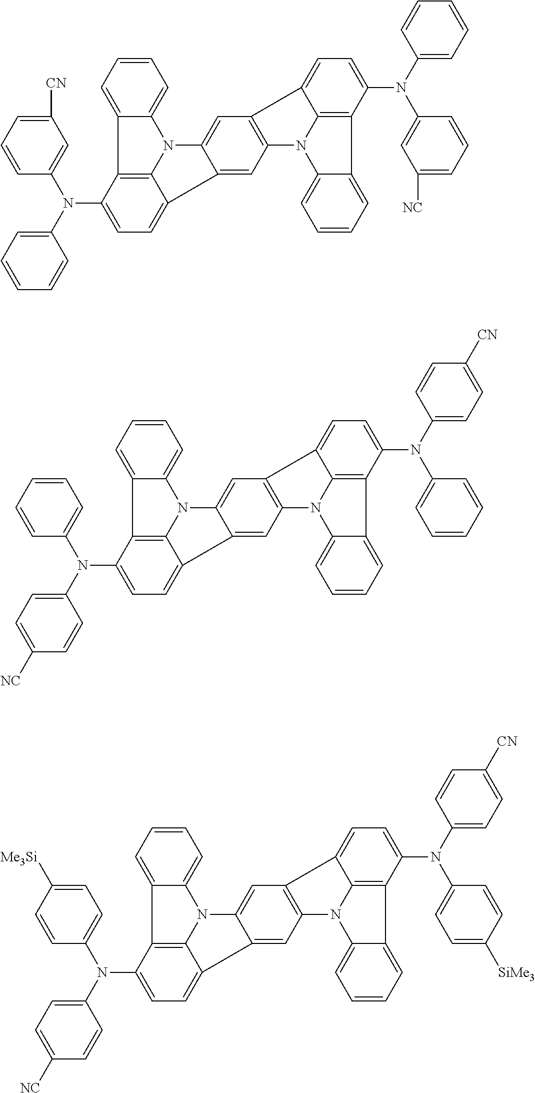 [SMILES: N#Cc1cccc(N(c2ccccc2)c2ccc3c4cc5c(cc4n4c6ccccc6c2c34)c2ccc(N(c3ccccc3)c3cccc(C#N)c3)c3c4ccccc4n5c23)c1 ; N#Cc1ccc(N(c2ccccc2)c2ccc3c4cc5c(cc4n4c6ccccc6c2c34)c2ccc(N(c3ccccc3)c3ccc(C#N)cc3)c3c4ccccc4n5c23)cc1 ; C[Si](C)(C)c1ccc(N(c2ccc(C#N)cc2)c2ccc3c4cc5c(cc4n4c6ccccc6c2c34)c2ccc(N(c3ccc(C#N)cc3)c3ccc([Si](C)(C)C)cc3)c3c4ccccc4n5c23)cc1]

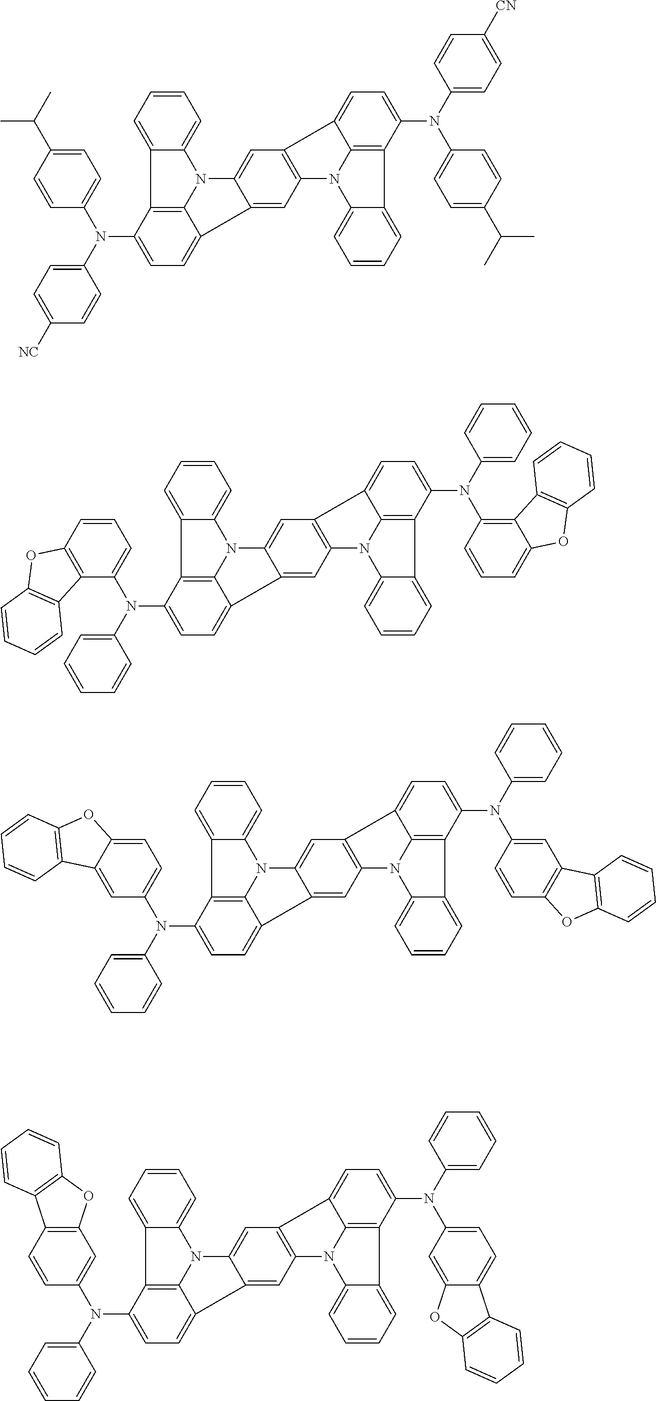 [SMILES: CC(C)c1ccc(N(c2ccc(C#N)cc2)c2ccc3c4cc5c(cc4n4c6ccccc6c2c34)c2ccc(N(c3ccc(C#N)cc3)c3ccc(C(C)C)cc3)c3c4ccccc4n5c23)cc1 ; c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1]

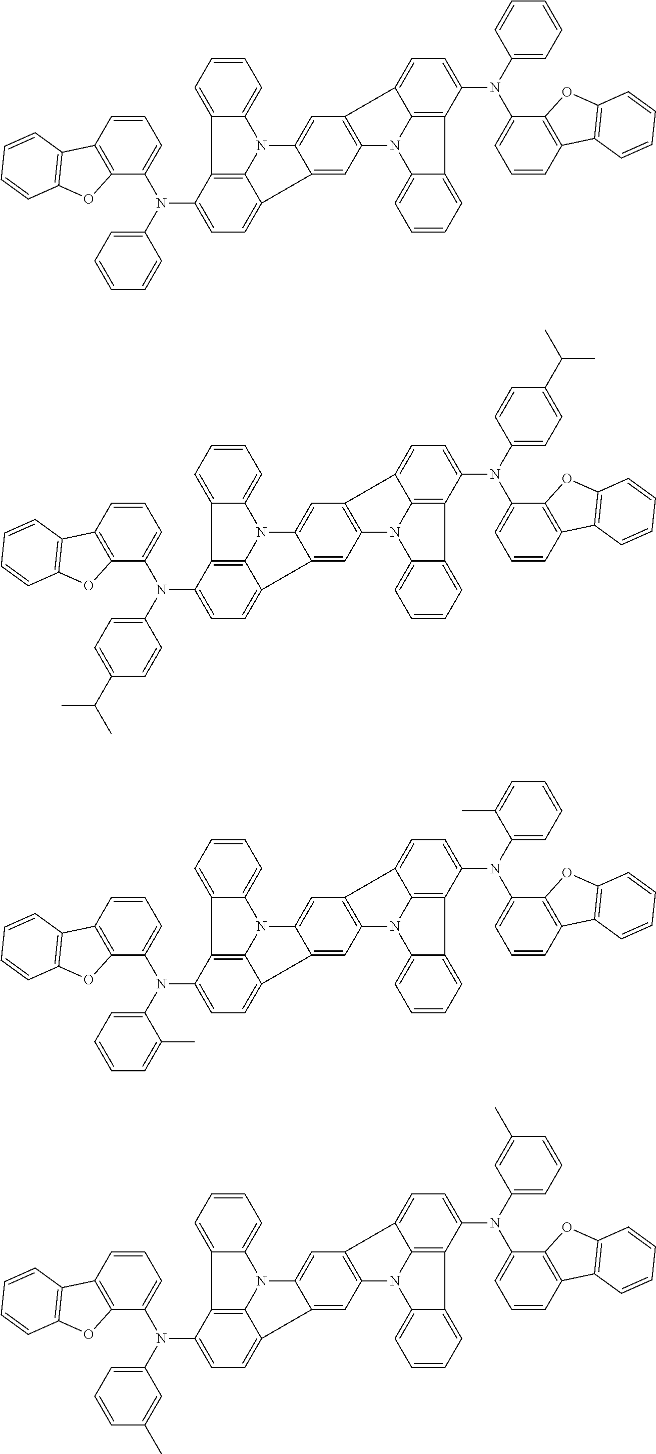 [SMILES: c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1 ; Cc1ccccc1N(c1cccc2c1oc1ccccc12)c1ccc2c3cc4c(cc3n3c5ccccc5c1c23)c1ccc(N(c2ccccc2C)c2cccc3c2oc2ccccc23)c2c3ccccc3n4c12 ; Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1]

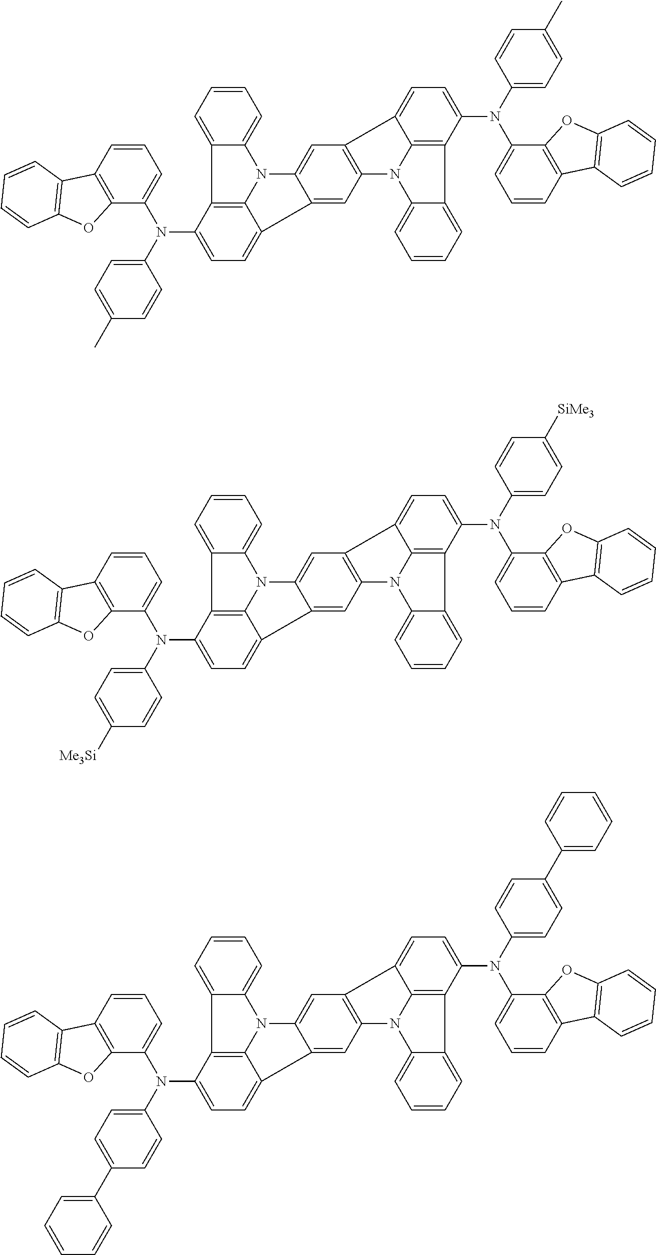 [SMILES: Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1 ; C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1 ; c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1]

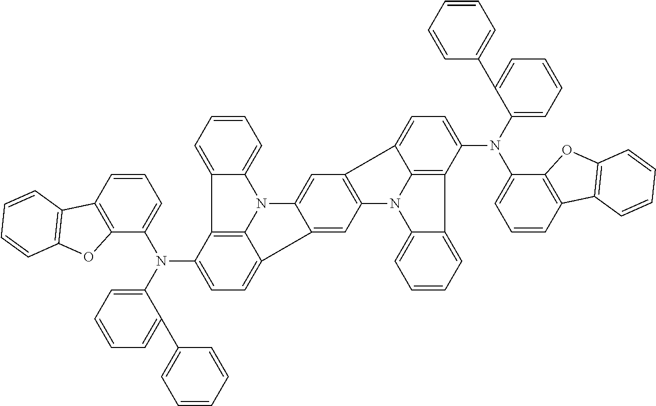 [SMILES: c1ccc(-c2ccccc2N(c2cccc3c2oc2ccccc23)c2ccc3c4cc5c(cc4n4c6ccccc6c2c34)c2ccc(N(c3ccccc3-c3ccccc3)c3cccc4c3oc3ccccc34)c3c4ccccc4n5c23)cc1]

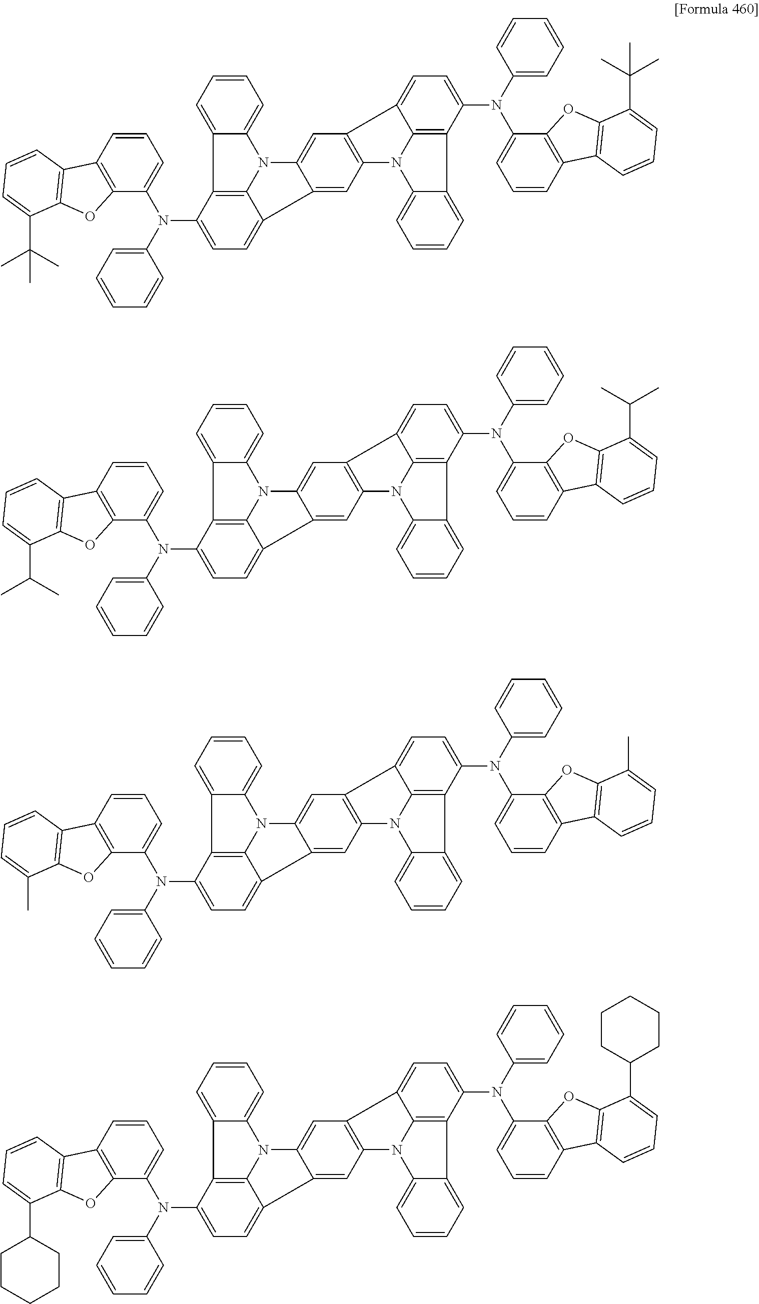

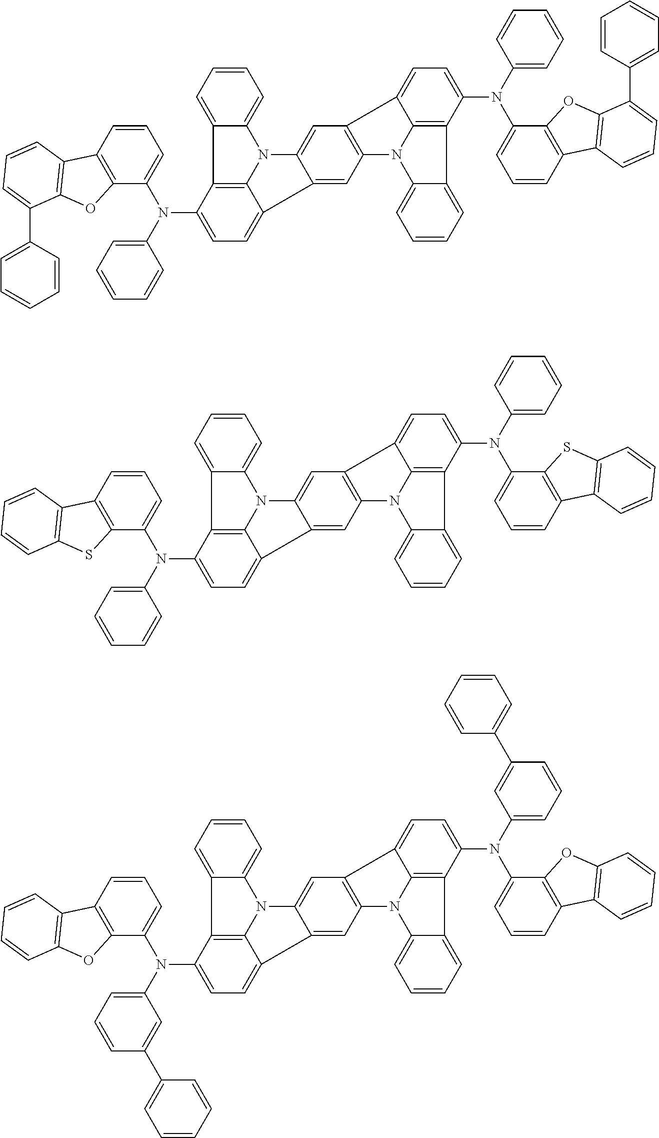 [SMILES: c1ccc(-c2cccc3c2oc2c(N(c4ccccc4)c4ccc5c6cc7c(cc6n6c8ccccc8c4c56)c4ccc(N(c5ccccc5)c5cccc6c5oc5c(-c8ccccc8)cccc56)c5c6ccccc6n7c45)cccc23)cc1 ; c1ccc(N(c2cccc3c2sc2ccccc23)c2ccc3c4cc5c(cc4n4c6ccccc6c2c34)c2ccc(N(c3ccccc3)c3cccc4c3sc3ccccc34)c3c4ccccc4n5c23)cc1 ; c1ccc(-c2cccc(N(c3cccc4c3oc3ccccc34)c3ccc4c5cc6c(cc5n5c7ccccc7c3c45)c3ccc(N(c4cccc(-c5ccccc5)c4)c4cccc5c4oc4ccccc45)c4c5ccccc5n6c34)c2)cc1]

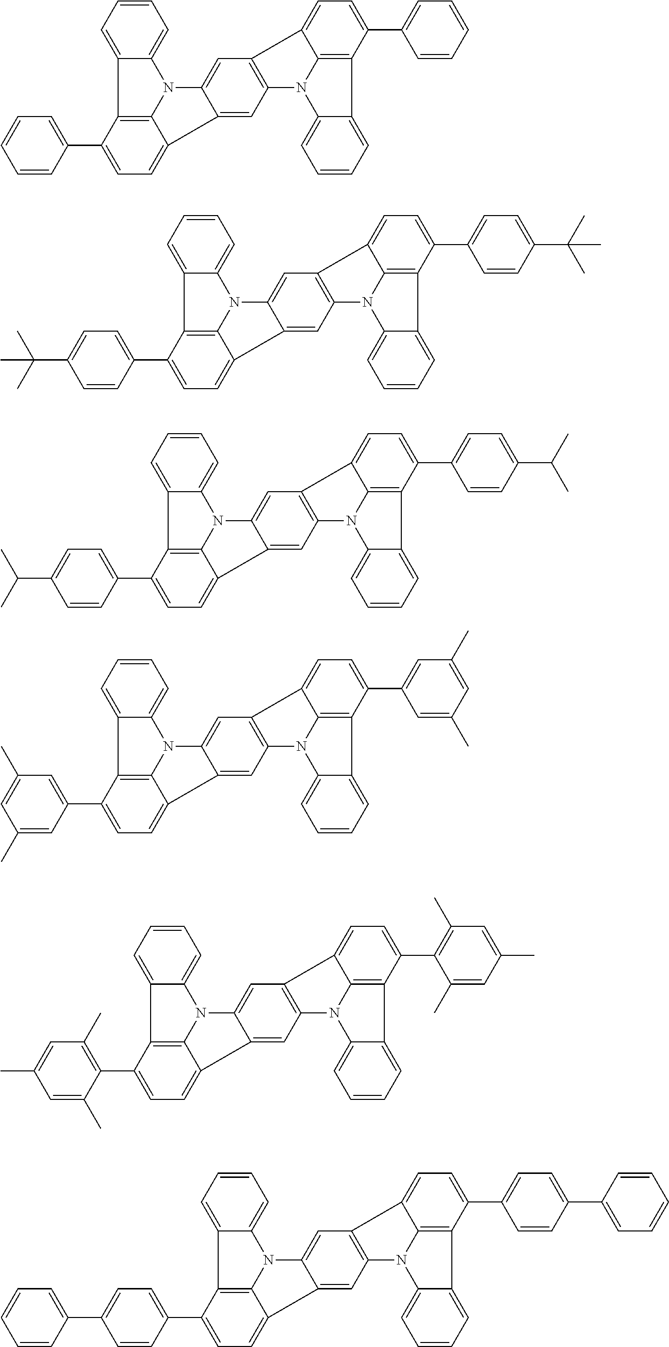

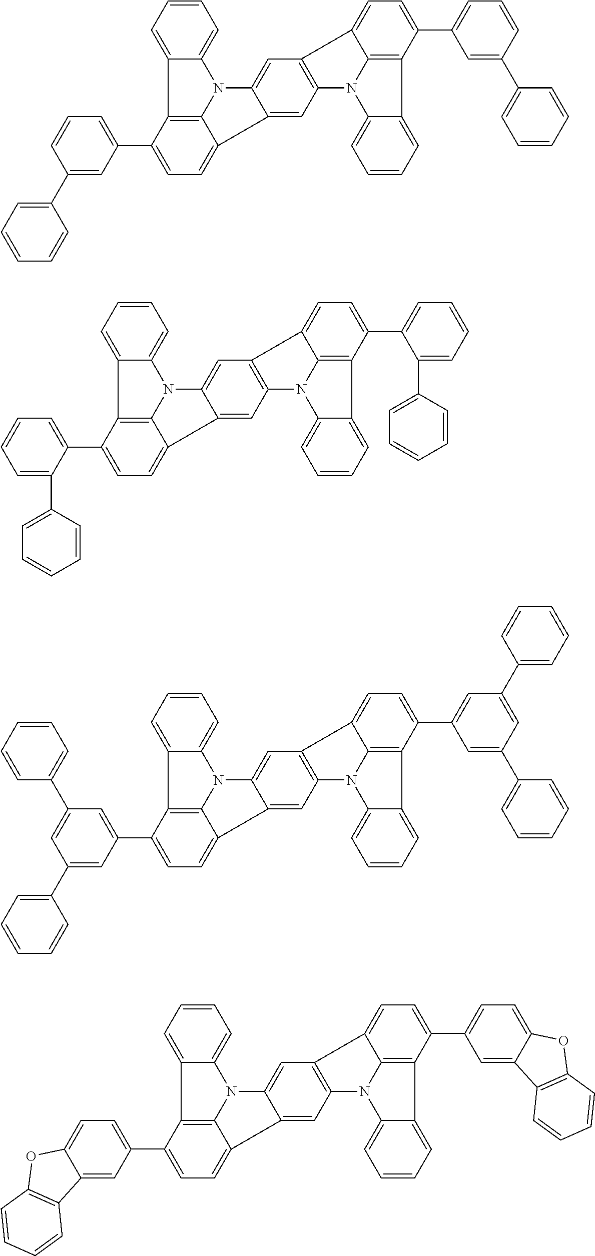 [SMILES: c1ccc(-c2cccc(-c3ccc4c5cc6c(cc5n5c7ccccc7c3c45)c3ccc(-c4cccc(-c5ccccc5)c4)c4c5ccccc5n6c34)c2)cc1 ; c1ccc(-c2ccccc2-c2ccc3c4cc5c(cc4n4c6ccccc6c2c34)c2ccc(-c3ccccc3-c3ccccc3)c3c4ccccc4n5c23)cc1 ; c1ccc(-c2cc(-c3ccccc3)cc(-c3ccc4c5cc6c(cc5n5c7ccccc7c3c45)c3ccc(-c4cc(-c5ccccc5)cc(-c5ccccc5)c4)c4c5ccccc5n6c34)c2)cc1 ; c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12]

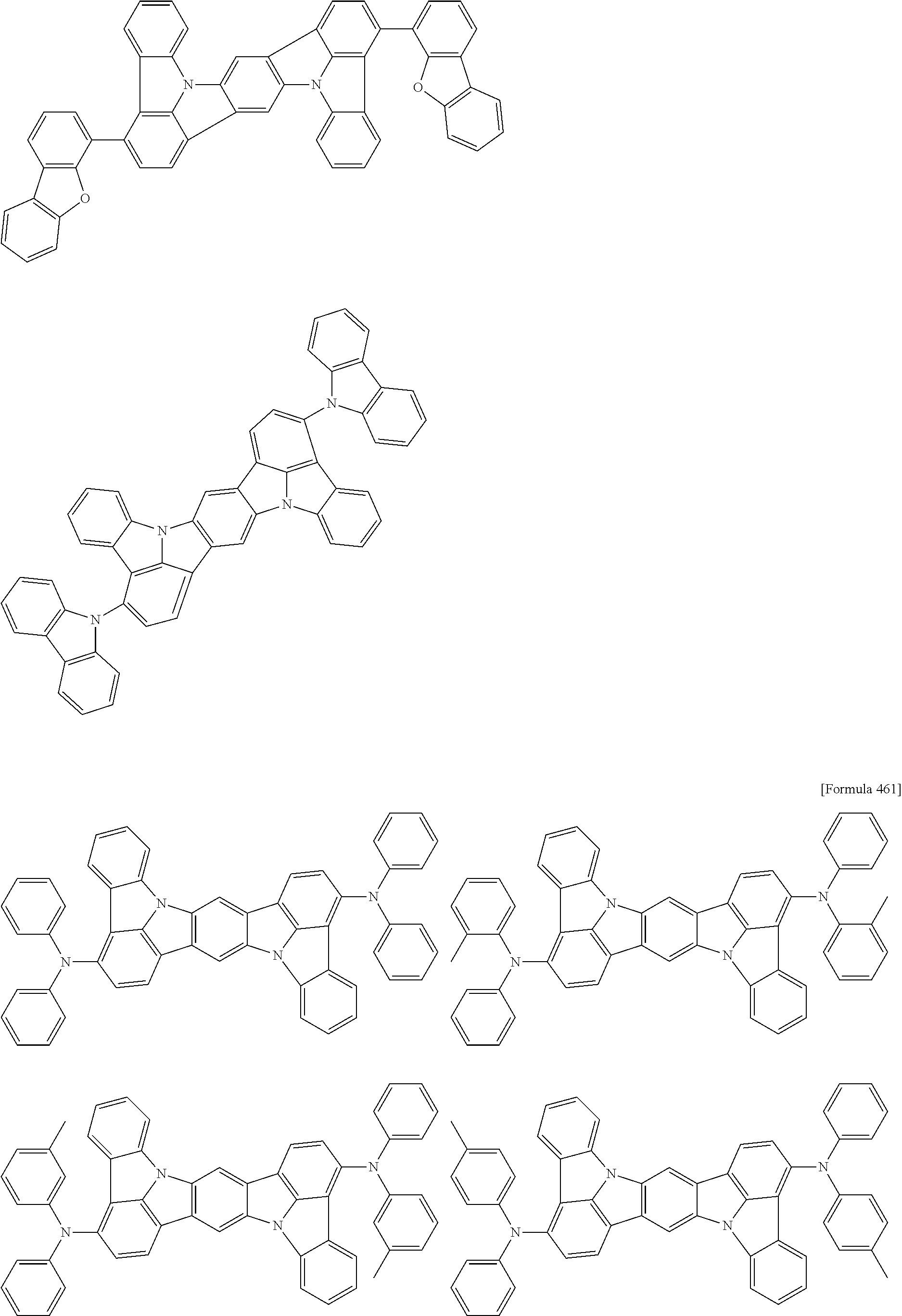

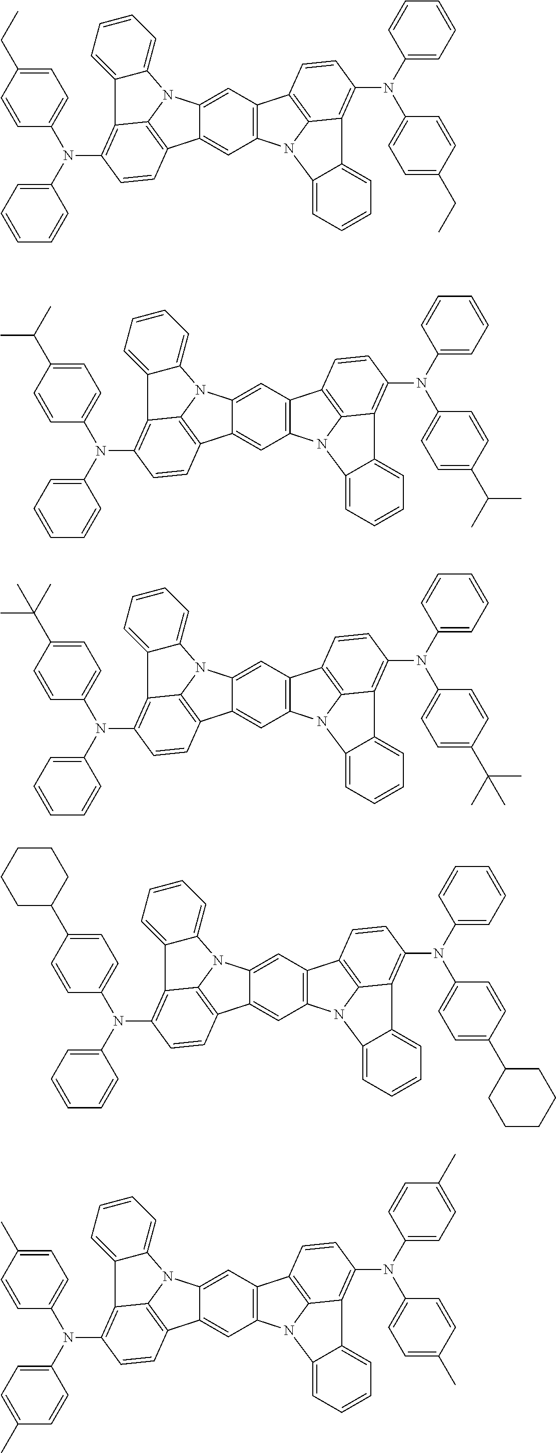

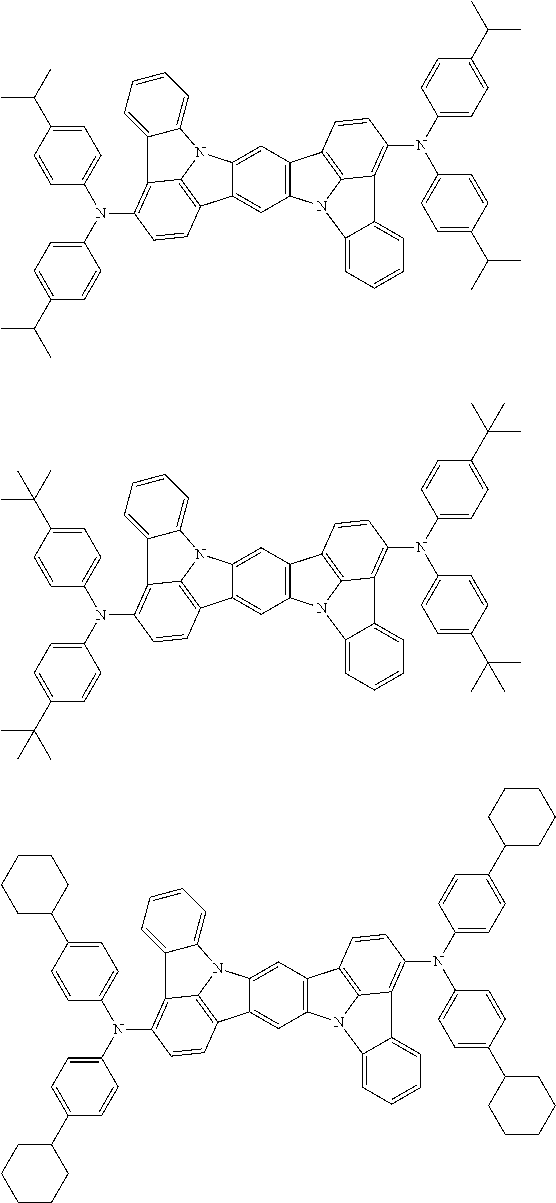

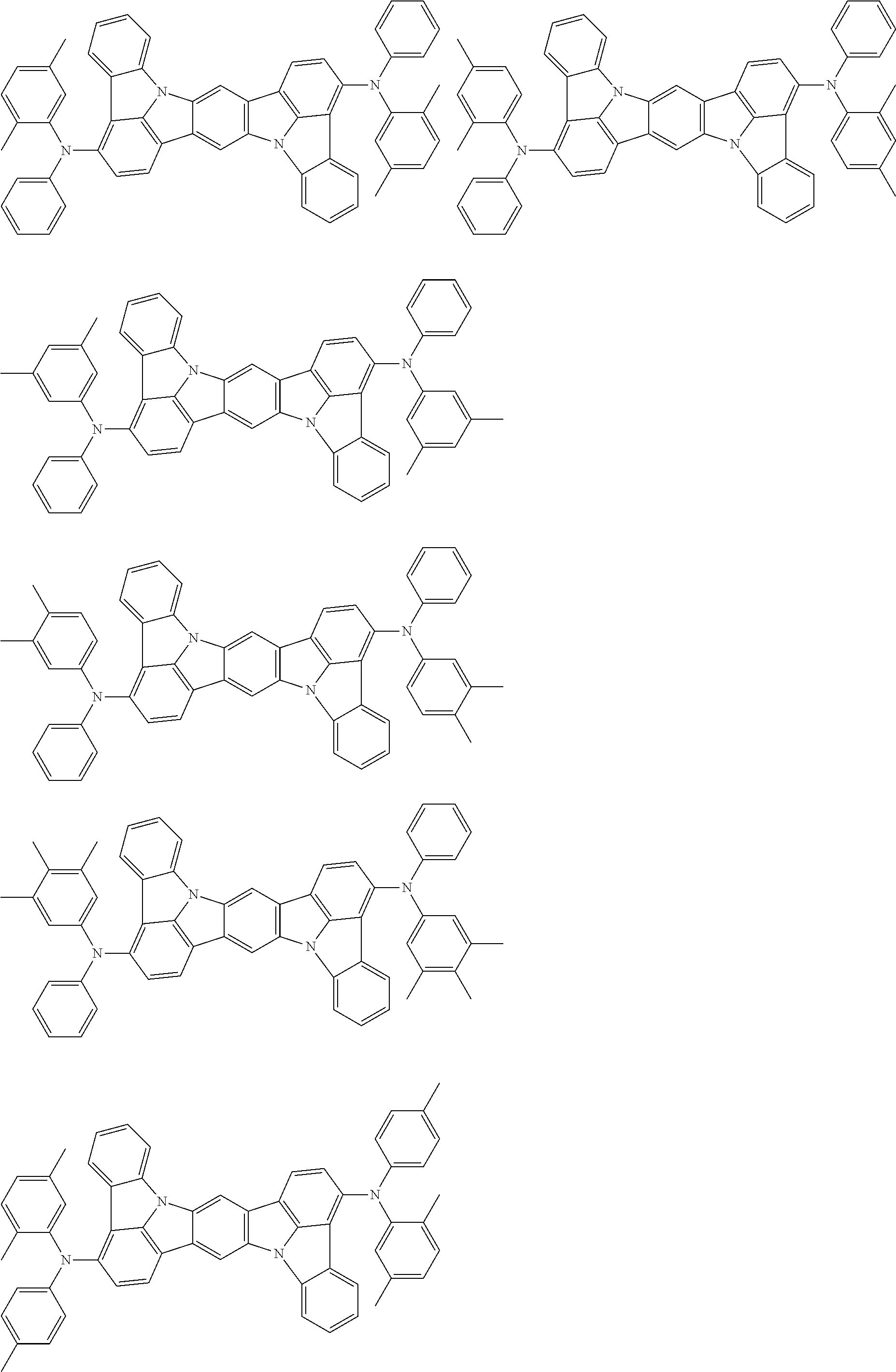

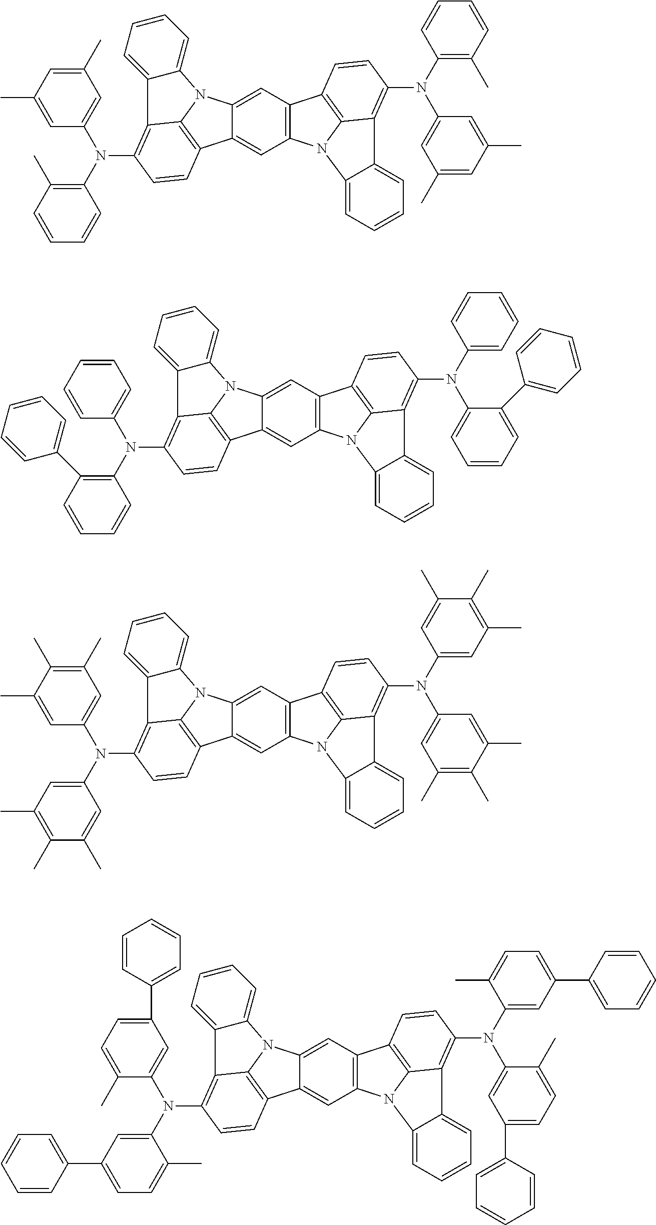

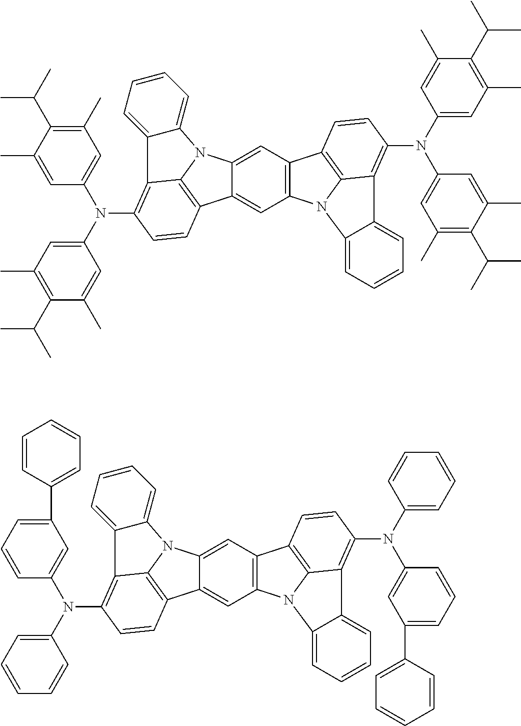 [SMILES: Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

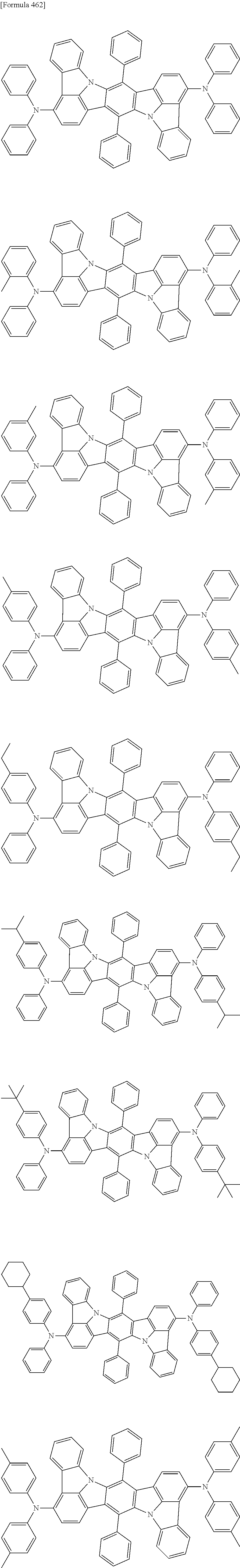

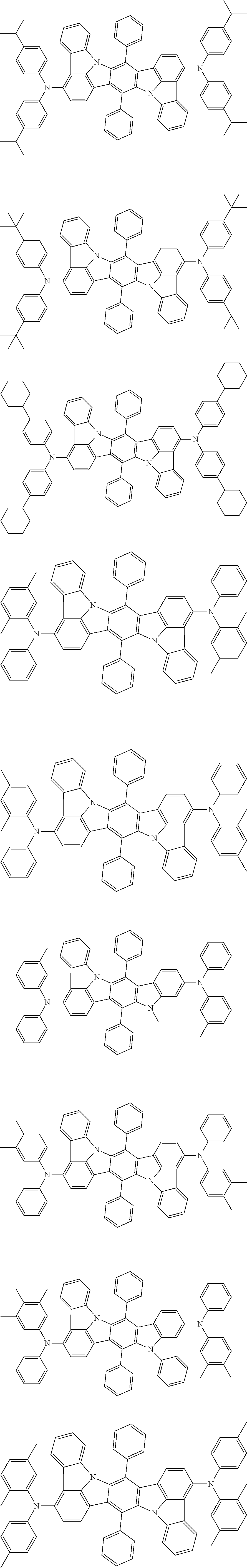

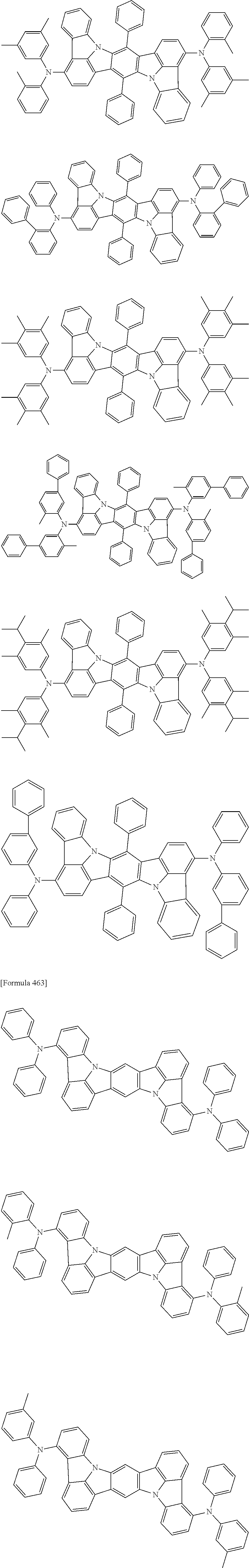

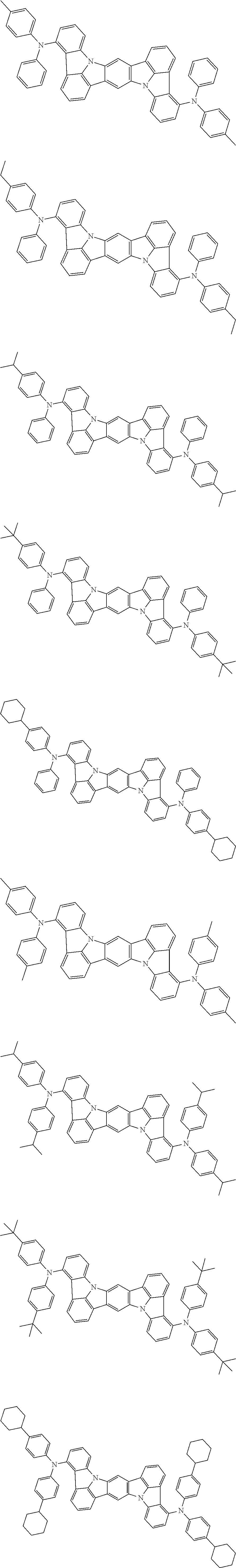

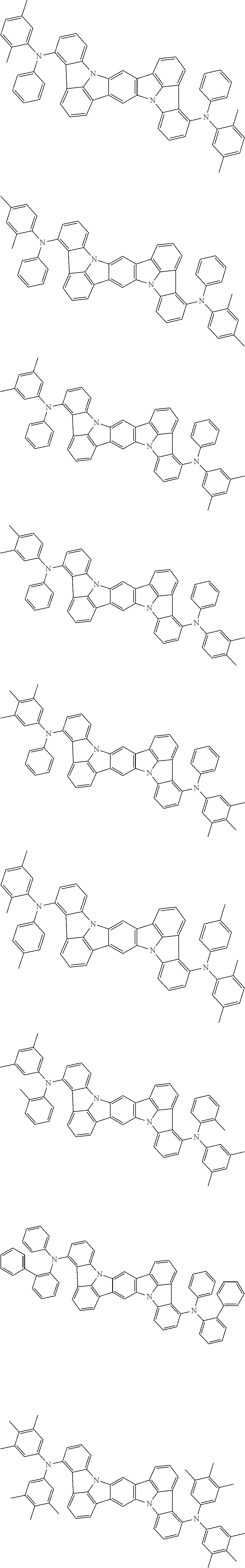

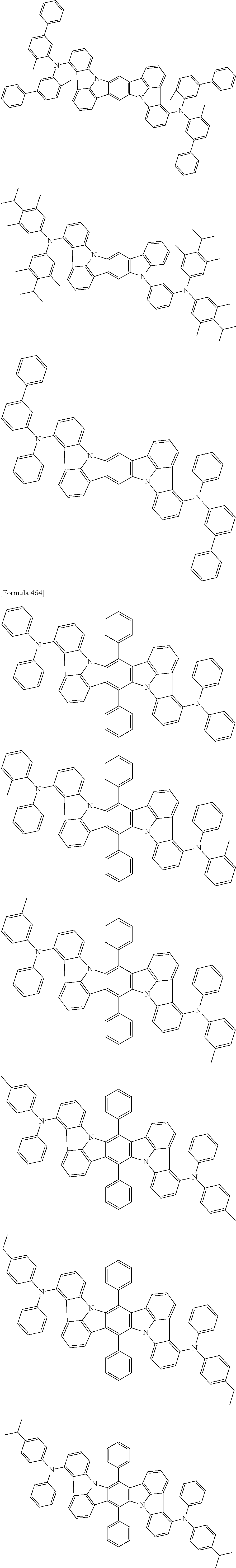

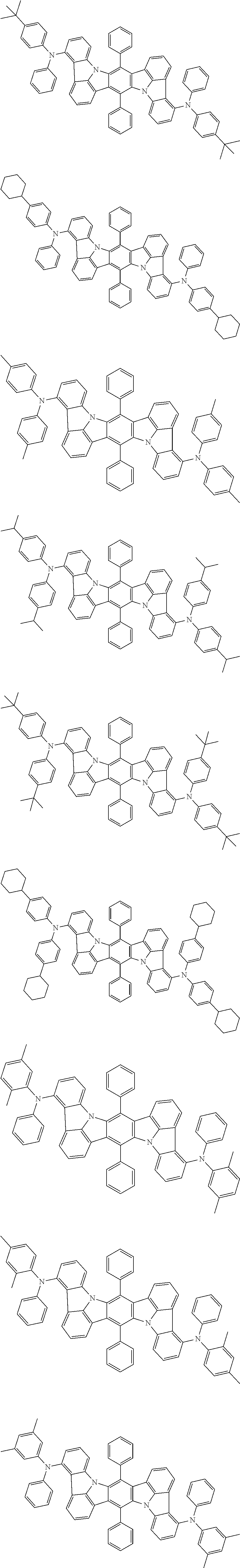

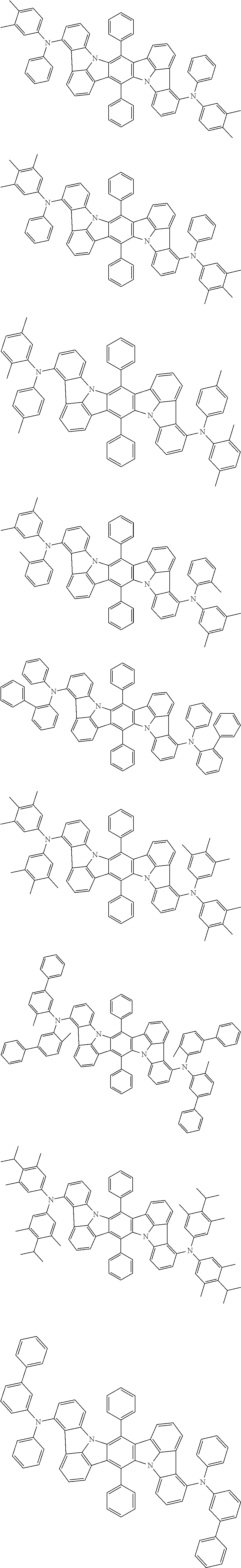

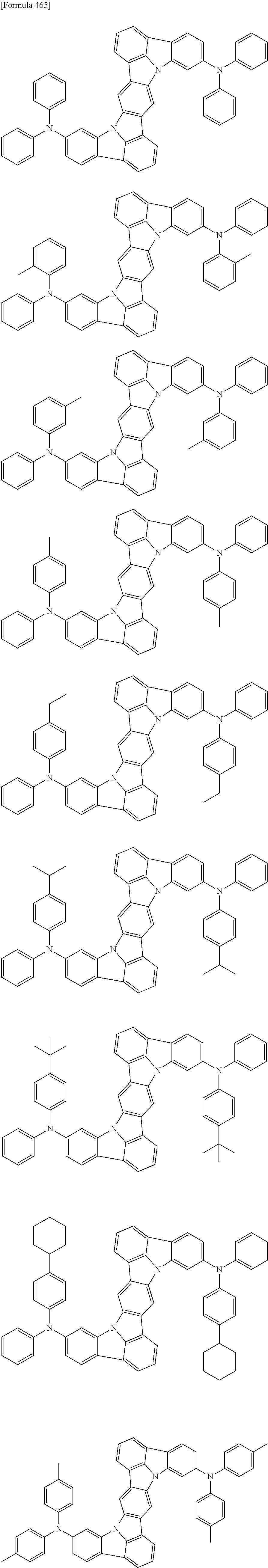

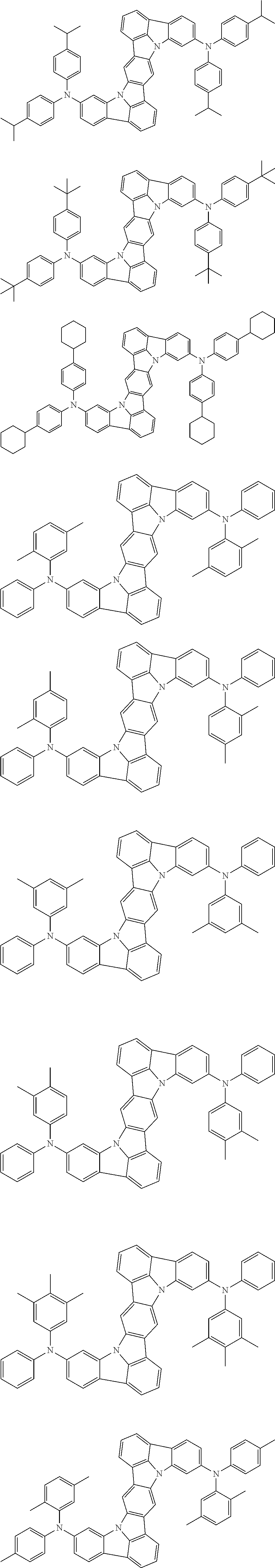

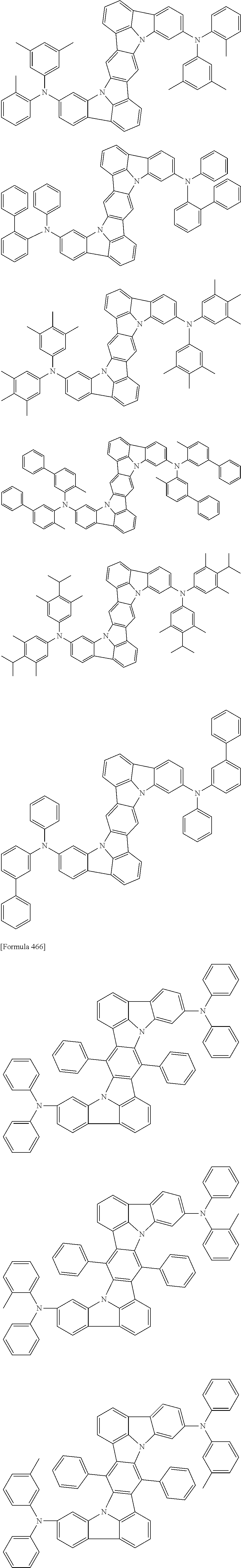

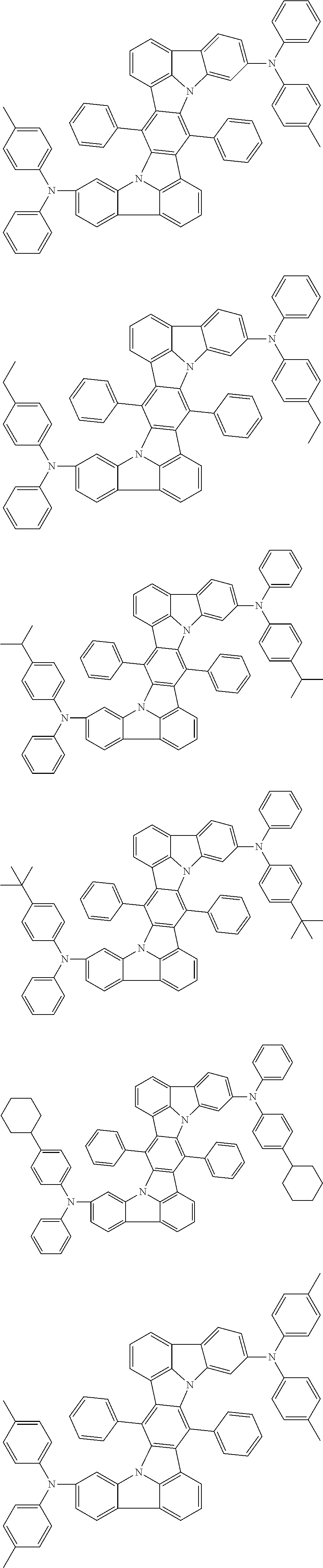

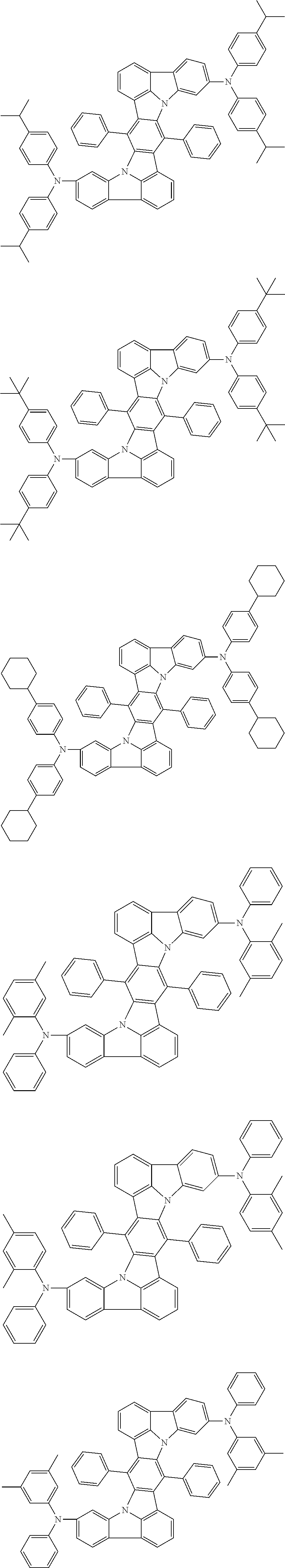

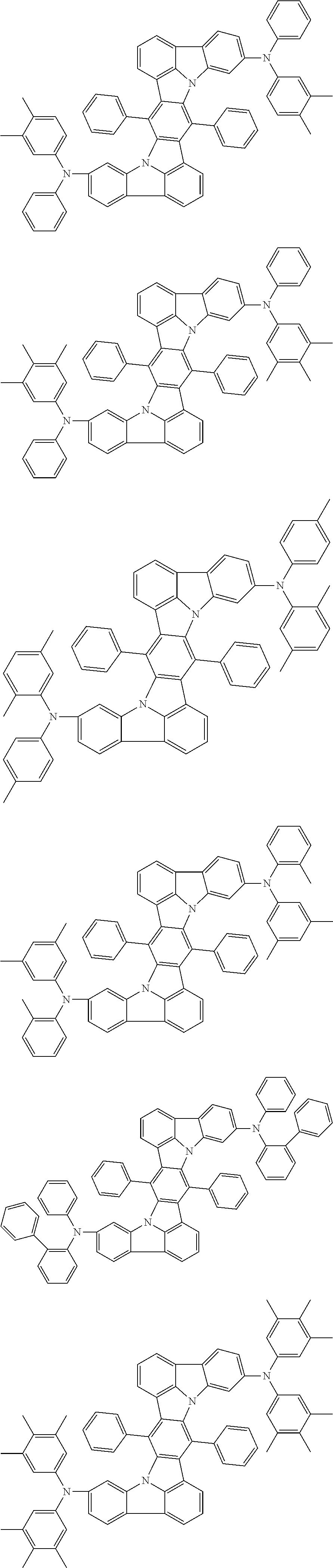

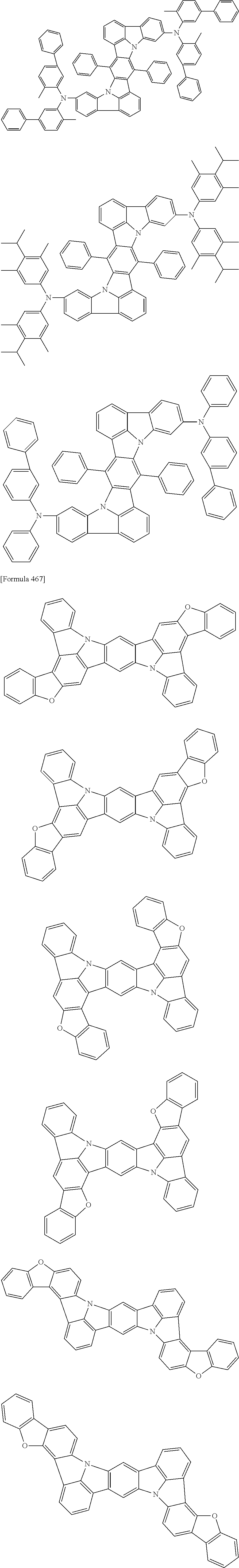

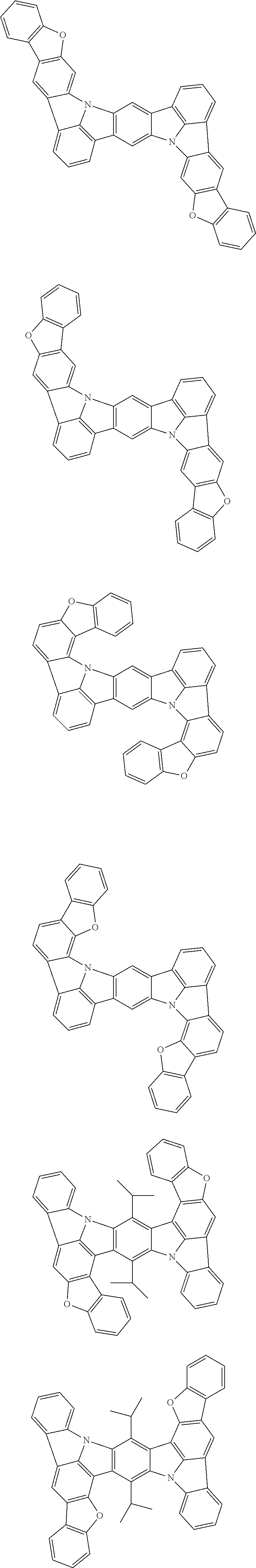

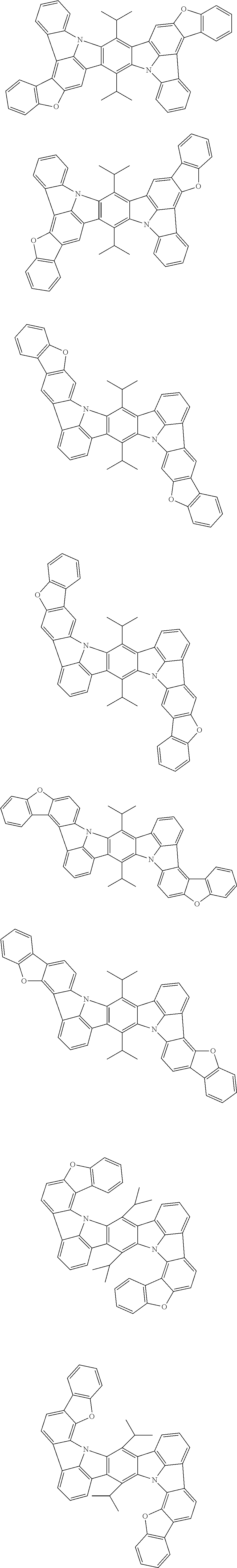

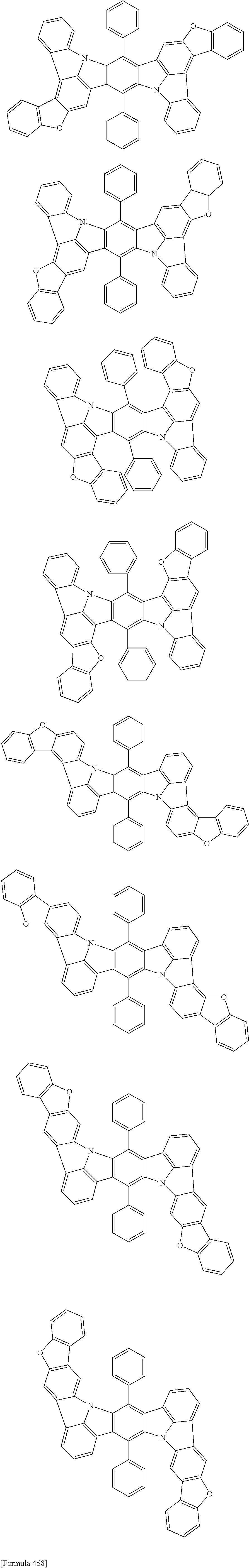

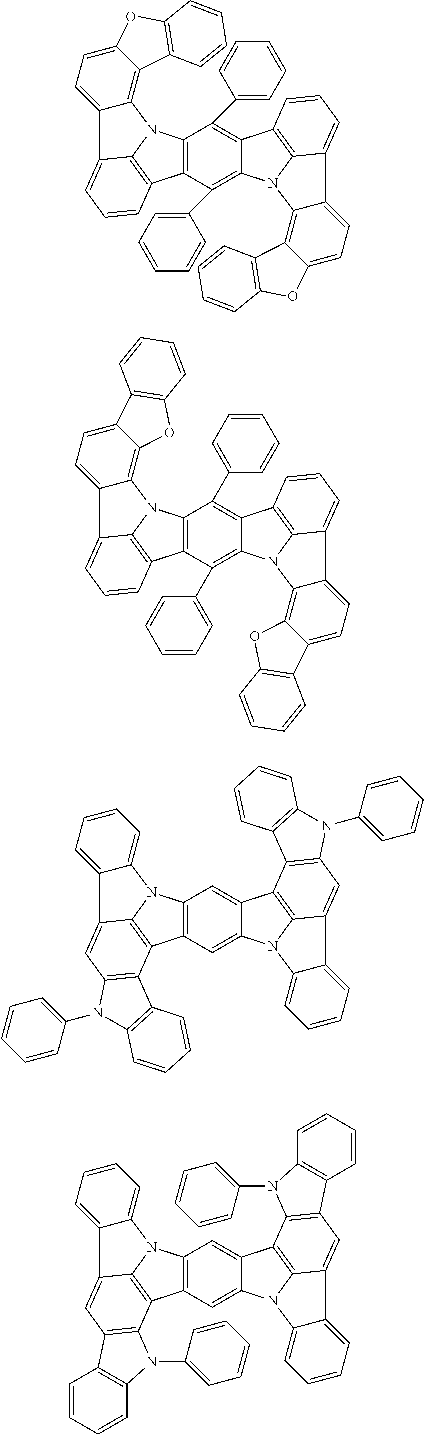

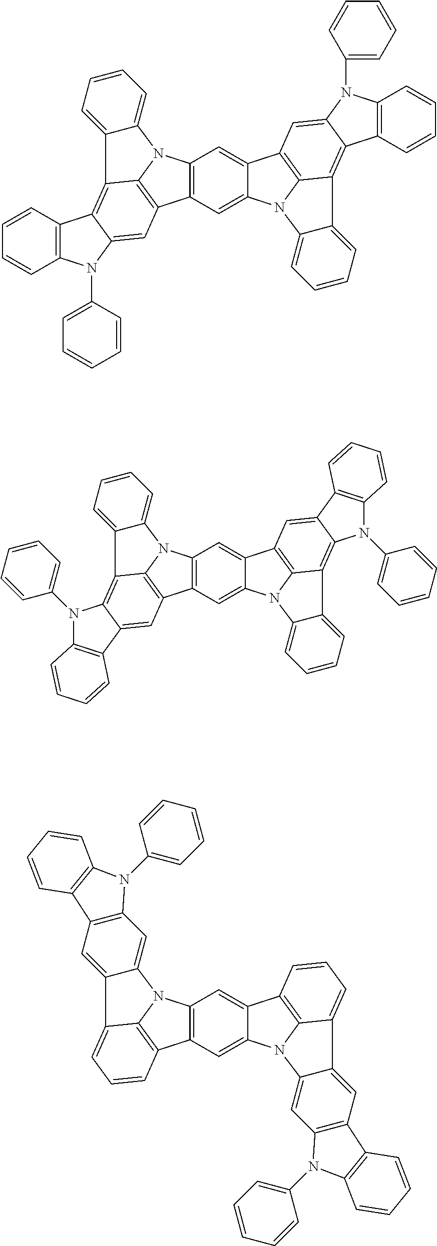 [SMILES: c1ccc(-n2c3ccccc3c3c4c5ccccc5n5c6cc7c8cc9c(c%10ccccc%10n9-c9ccccc9)c9c%10ccccc%10n(c7cc6c(cc32)c45)c89)cc1 ; c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1 ; c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1]

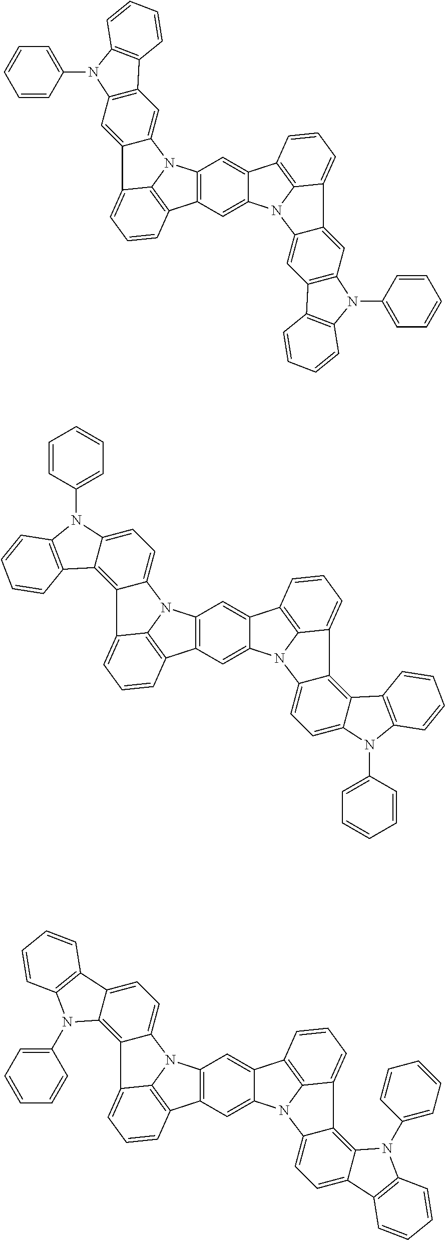 [SMILES: c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1 ; c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1 ; c1ccc(-n2c3ccccc3c3ccc4c(c5cccc6c7cc8c(cc7n4c65)c4cccc5c6c7c(ccc6n8c45)c4ccccc4n7-c4ccccc4)c32)cc1]

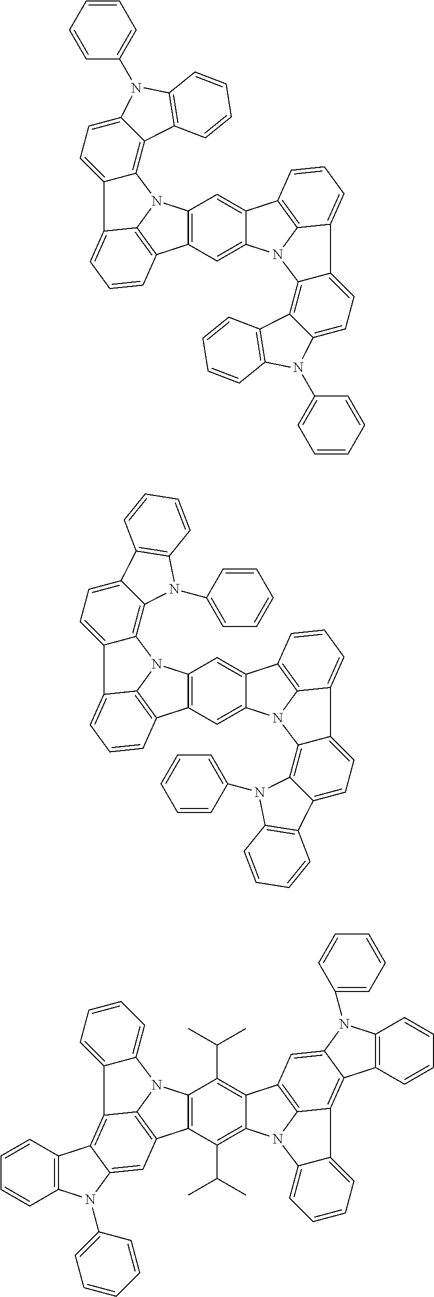 [SMILES: c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1 ; c1ccc(-n2c3ccccc3c3ccc4c5cccc6c7cc8c(cc7n(c65)c4c32)c2cccc3c4ccc5c6ccccc6n(-c6ccccc6)c5c4n8c23)cc1 ; CC(C)c1c2c3cc4c(c5ccccc5n4-c4ccccc4)c4c5ccccc5n(c2c(C(C)C)c2c5cc6c(c7ccccc7n6-c6ccccc6)c6c7ccccc7n(c12)c56)c34]

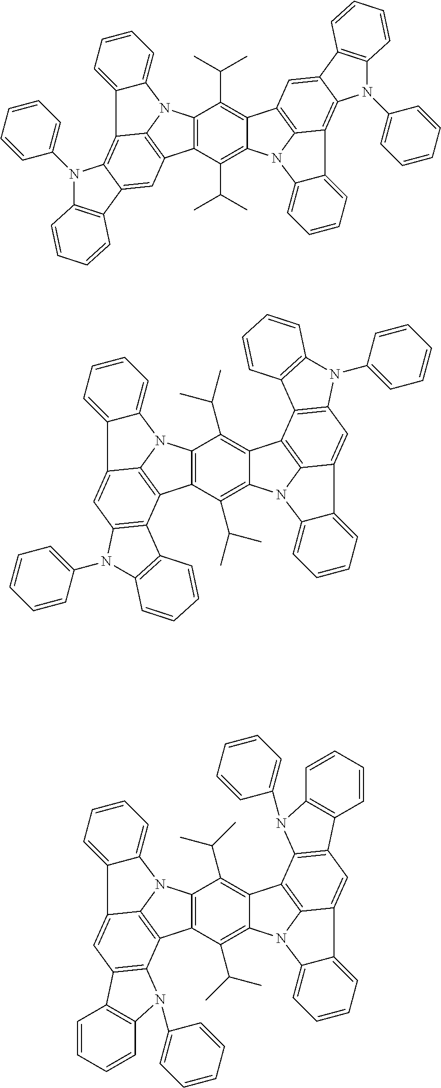 [SMILES: CC(C)c1c2c3cc4c5ccccc5n(-c5ccccc5)c4c4c5ccccc5n(c2c(C(C)C)c2c5cc6c7ccccc7n(-c7ccccc7)c6c6c7ccccc7n(c12)c56)c34 ; CC(C)c1c2c3c4c5ccccc5n(-c5ccccc5)c4cc4c5ccccc5n(c2c(C(C)C)c2c5c6c7ccccc7n(-c7ccccc7)c6cc6c7ccccc7n(c12)c65)c43 ; CC(C)c1c2c3c4c(cc5c6ccccc6n(c2c(C(C)C)c2c6c7c(cc8c9ccccc9n(c12)c86)c1ccccc1n7-c1ccccc1)c53)c1ccccc1n4-c1ccccc1]

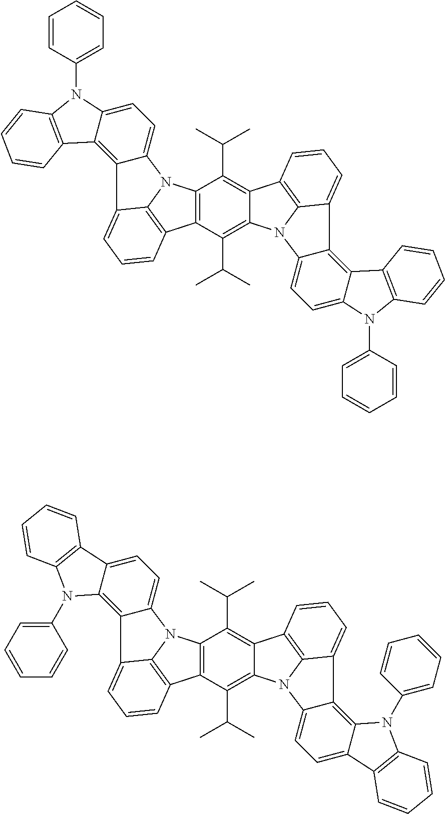 [SMILES: CC(C)c1c2c3cccc4c5c6c7ccccc7n(-c7ccccc7)c6ccc5n(c2c(C(C)C)c2c5cccc6c7c8c9ccccc9n(-c9ccccc9)c8ccc7n(c12)c56)c34 ; CC(C)c1c2c3cccc4c5c6c(ccc5n(c2c(C(C)C)c2c5cccc7c8c9c(ccc8n(c12)c57)c1ccccc1n9-c1ccccc1)c34)c1ccccc1n6-c1ccccc1]

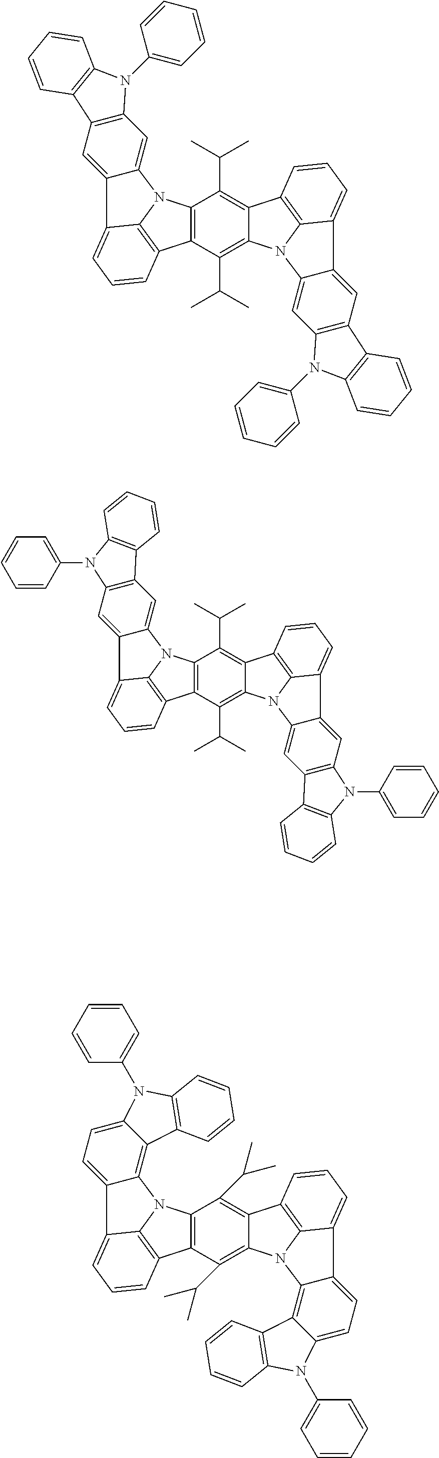 [SMILES: CC(C)c1c2c3cccc4c5cc6c7ccccc7n(-c7ccccc7)c6cc5n(c2c(C(C)C)c2c5cccc6c7cc8c9ccccc9n(-c9ccccc9)c8cc7n(c12)c65)c43 ; CC(C)c1c2c3cccc4c5cc6c(cc5n(c2c(C(C)C)c2c5cccc7c8cc9c(cc8n(c12)c75)c1ccccc1n9-c1ccccc1)c43)c1ccccc1n6-c1ccccc1 ; CC(C)c1c2c3cccc4c5ccc6c(c7ccccc7n6-c6ccccc6)c5n(c2c(C(C)C)c2c5cccc6c7ccc8c(c9ccccc9n8-c8ccccc8)c7n(c12)c65)c43]

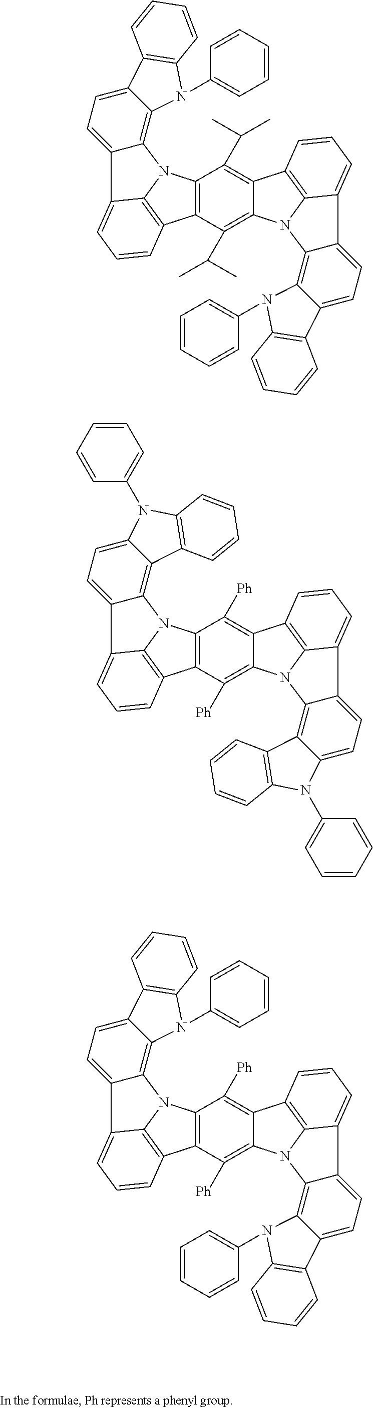 [SMILES: CC(C)c1c2c3cccc4c5ccc6c7ccccc7n(-c7ccccc7)c6c5n(c2c(C(C)C)c2c5cccc6c7ccc8c9ccccc9n(-c9ccccc9)c8c7n(c12)c65)c43 ; c1ccc(-c2c3c4cccc5c6ccc7c(c8ccccc8n7-c7ccccc7)c6n(c3c(-c3ccccc3)c3c6cccc7c8ccc9c(c%10ccccc%10n9-c9ccccc9)c8n(c23)c76)c54)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1]

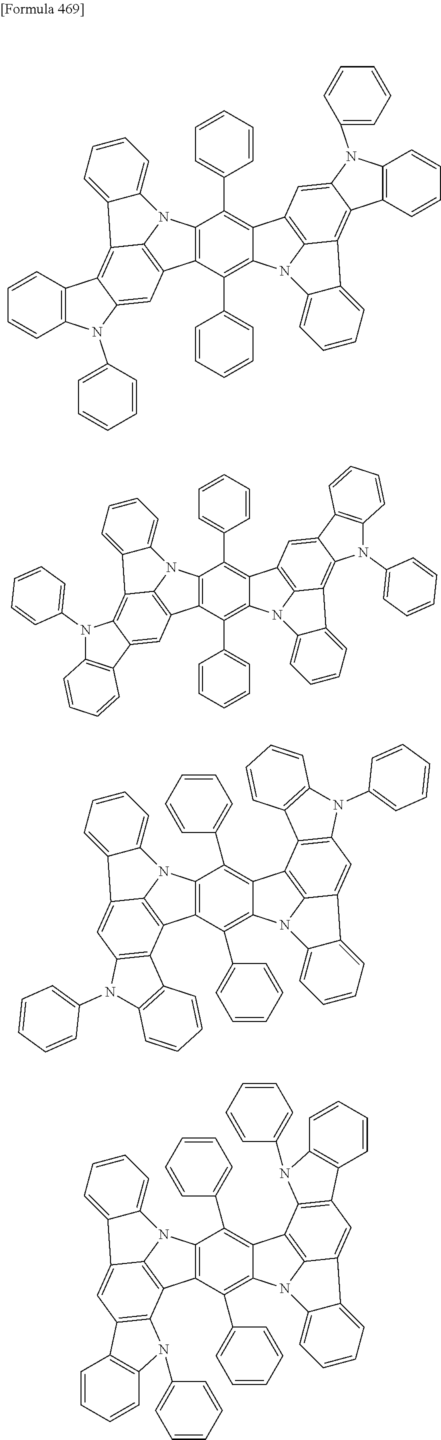 [SMILES: c1ccc(-c2c3c4cc5c(c6ccccc6n5-c5ccccc5)c5c6ccccc6n(c3c(-c3ccccc3)c3c6cc7c(c8ccccc8n7-c7ccccc7)c7c8ccccc8n(c23)c67)c45)cc1 ; c1ccc(-c2c3c4cc5c6ccccc6n(-c6ccccc6)c5c5c6ccccc6n(c3c(-c3ccccc3)c3c6cc7c8ccccc8n(-c8ccccc8)c7c7c8ccccc8n(c23)c67)c45)cc1 ; c1ccc(-c2c3c4c5c6ccccc6n(-c6ccccc6)c5cc5c6ccccc6n(c3c(-c3ccccc3)c3c6c7c8ccccc8n(-c8ccccc8)c7cc7c8ccccc8n(c23)c76)c54)cc1 ; c1ccc(-c2c3c4c5c(cc6c7ccccc7n(c3c(-c3ccccc3)c3c7c8c(cc9c%10ccccc%10n(c23)c97)c2ccccc2n8-c2ccccc2)c64)c2ccccc2n5-c2ccccc2)cc1]

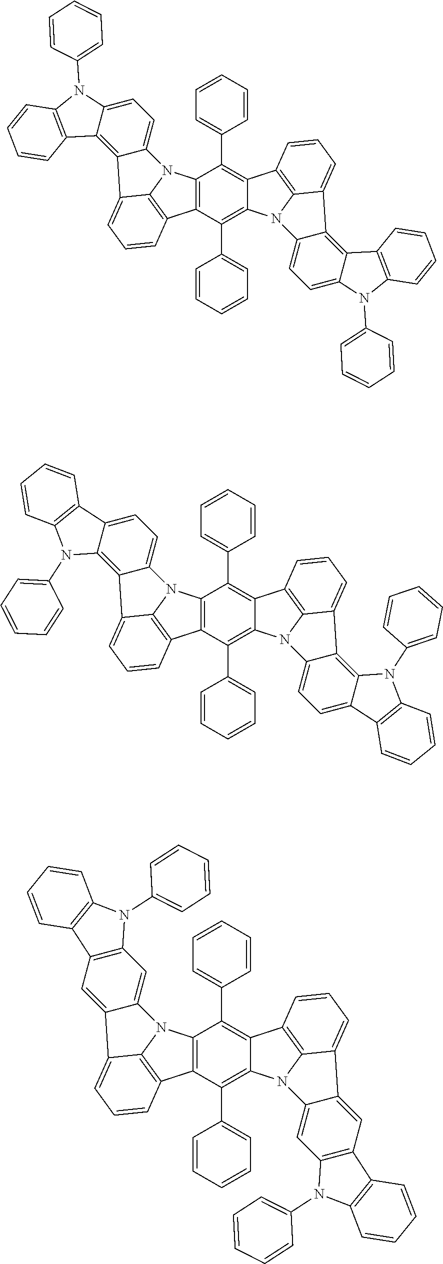 [SMILES: c1ccc(-c2c3c4cccc5c6c7c8ccccc8n(-c8ccccc8)c7ccc6n(c3c(-c3ccccc3)c3c6cccc7c8c9c%10ccccc%10n(-c%10ccccc%10)c9ccc8n(c23)c67)c45)cc1 ; c1ccc(-c2c3c4cccc5c6c7c(ccc6n(c3c(-c3ccccc3)c3c6cccc8c9c%10c(ccc9n(c23)c68)c2ccccc2n%10-c2ccccc2)c45)c2ccccc2n7-c2ccccc2)cc1 ; c1ccc(-c2c3c4cccc5c6cc7c8ccccc8n(-c8ccccc8)c7cc6n(c3c(-c3ccccc3)c3c6cccc7c8cc9c%10ccccc%10n(-c%10ccccc%10)c9cc8n(c23)c76)c54)cc1]

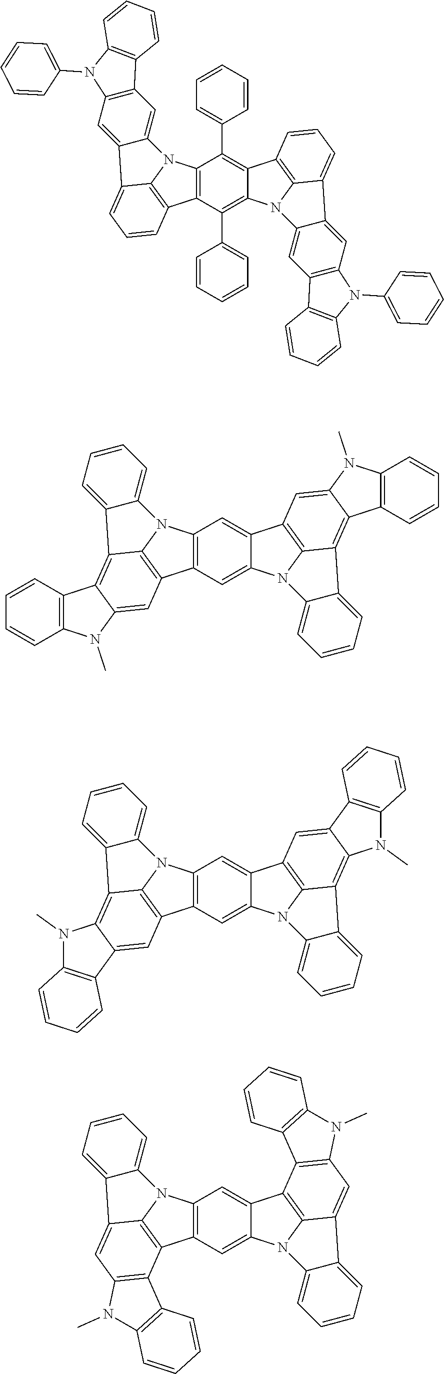

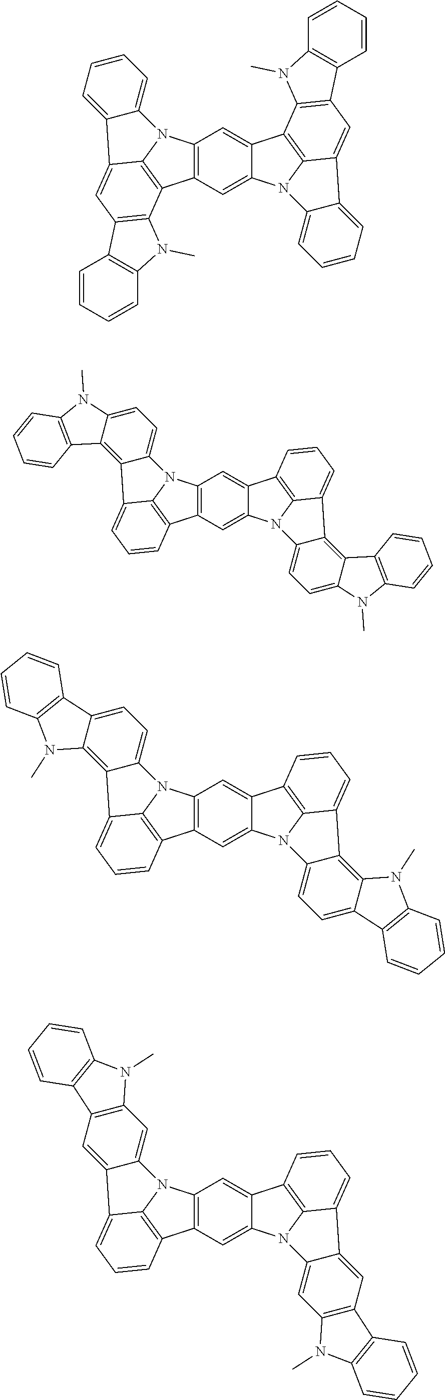 [SMILES: Cn1c2ccccc2c2cc3c4ccccc4n4c5cc6c7c8c(cc9c%10ccccc%10n(c6cc5c(c21)c34)c97)c1ccccc1n8C ; Cn1c2ccccc2c2c3c4cccc5c6cc7c(cc6n(c3ccc21)c54)c1cccc2c3c4c5ccccc5n(C)c4ccc3n7c12 ; Cn1c2ccccc2c2ccc3c(c4cccc5c6cc7c(cc6n3c54)c3cccc4c5c6c(ccc5n7c34)c3ccccc3n6C)c21 ; Cn1c2ccccc2c2cc3c4cccc5c6cc7c(cc6n(c3cc21)c54)c1cccc2c3cc4c5ccccc5n(C)c4cc3n7c12]

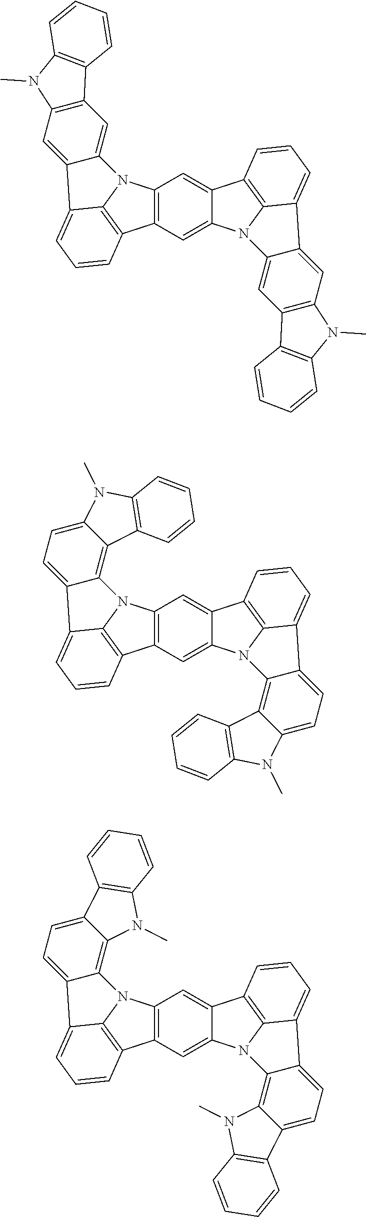

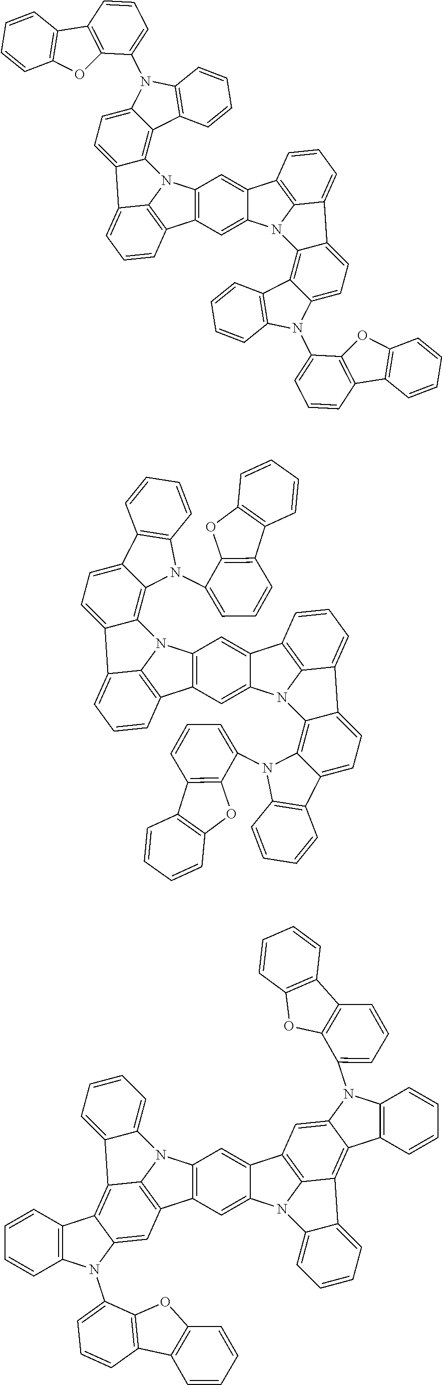 [SMILES: c1ccc2c(c1)oc1c(-n3c4ccccc4c4c3ccc3c5cccc6c7cc8c(cc7n(c65)c34)c3cccc4c5ccc6c(c7ccccc7n6-c6cccc7c6oc6ccccc67)c5n8c34)cccc12 ; c1ccc2c(c1)oc1c(-n3c4ccccc4c4ccc5c6cccc7c8cc9c(cc8n(c76)c5c43)c3cccc4c5ccc6c7ccccc7n(-c7cccc8c7oc7ccccc78)c6c5n9c34)cccc12 ; c1ccc2c(c1)oc1c(-n3c4ccccc4c4c5c6ccccc6n6c7cc8c9cc%10c(c%11ccccc%11n%10-c%10cccc%11c%10oc%10ccccc%10%11)c%10c%11ccccc%11n(c8cc7c(cc43)c56)c9%10)cccc12]

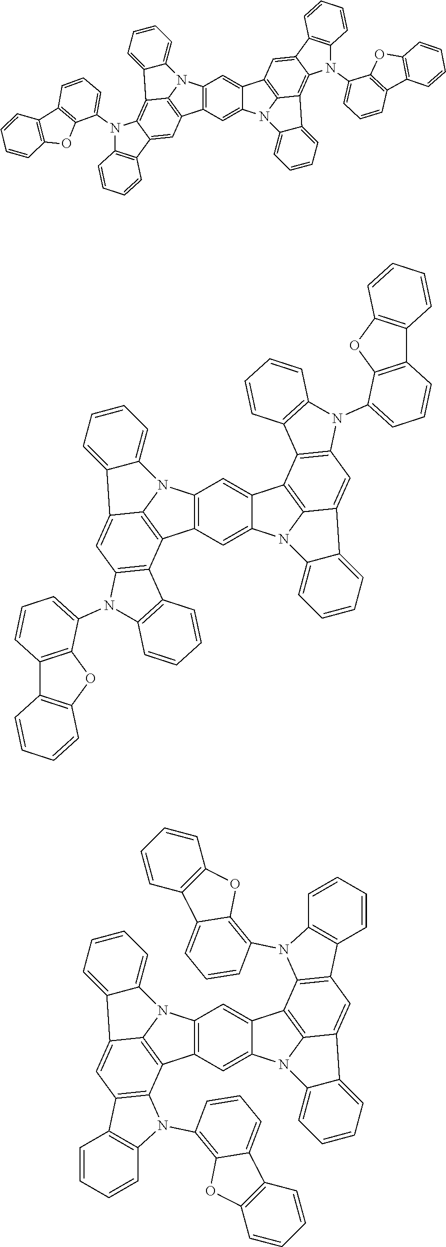 [SMILES: c1ccc2c(c1)oc1c(-n3c4ccccc4c4cc5c6cc7c(cc6n6c8ccccc8c(c43)c56)c3cc4c5ccccc5n(-c5cccc6c5oc5ccccc56)c4c4c5ccccc5n7c34)cccc12 ; c1ccc2c(c1)oc1c(-n3c4ccccc4c4c5c6cc7c(cc6n6c8ccccc8c(cc43)c56)c3c4c5ccccc5n(-c5cccc6c5oc5ccccc56)c4cc4c5ccccc5n7c43)cccc12 ; c1ccc2c(c1)oc1c(-n3c4ccccc4c4cc5c6ccccc6n6c7cc8c9c%10c(cc%11c%12ccccc%12n(c8cc7c(c43)c56)c%119)c3ccccc3n%10-c3cccc4c3oc3ccccc34)cccc12]

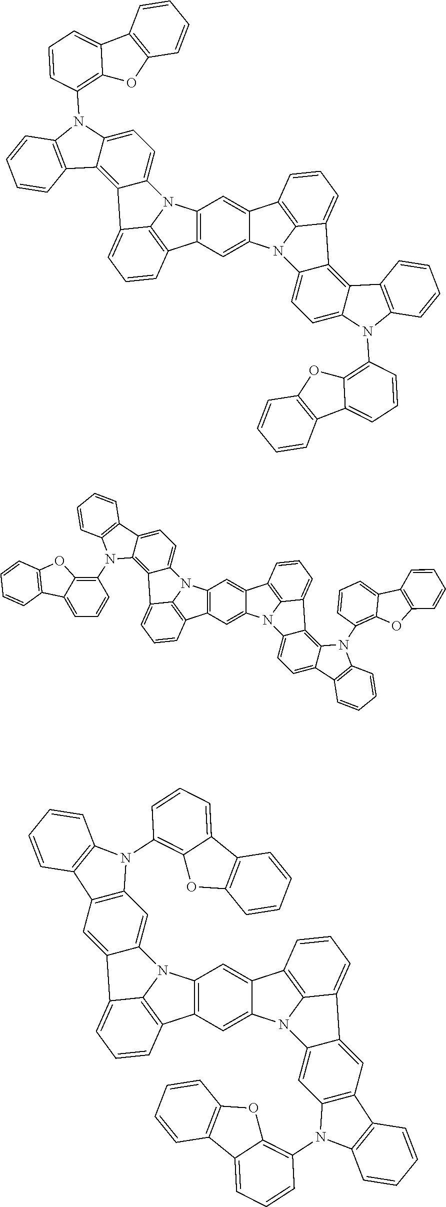 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12]

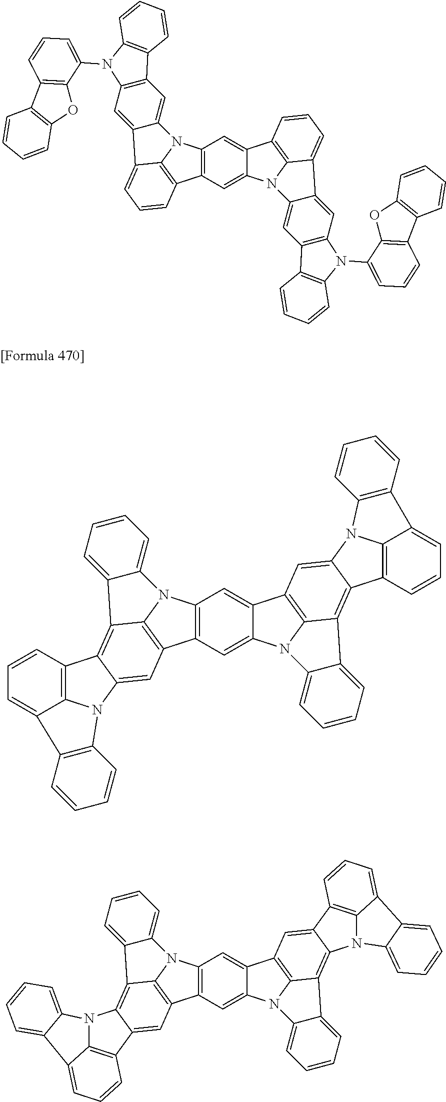 [SMILES: c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12 ; c1ccc2c(c1)c1cccc3c4c5c6ccccc6n6c7cc8c9cc%10c(c%11cccc%12c%13ccccc%13n%10c%12%11)c%10c%11ccccc%11n(c8cc7c(cc4n2c13)c56)c9%10 ; c1ccc2c(c1)c1c3c(cc4c5cccc6c7ccccc7n(c65)c41)c1cc4c(cc1n23)c1cc2c3cccc5c6ccccc6n(c53)c2c2c3ccccc3n4c12]

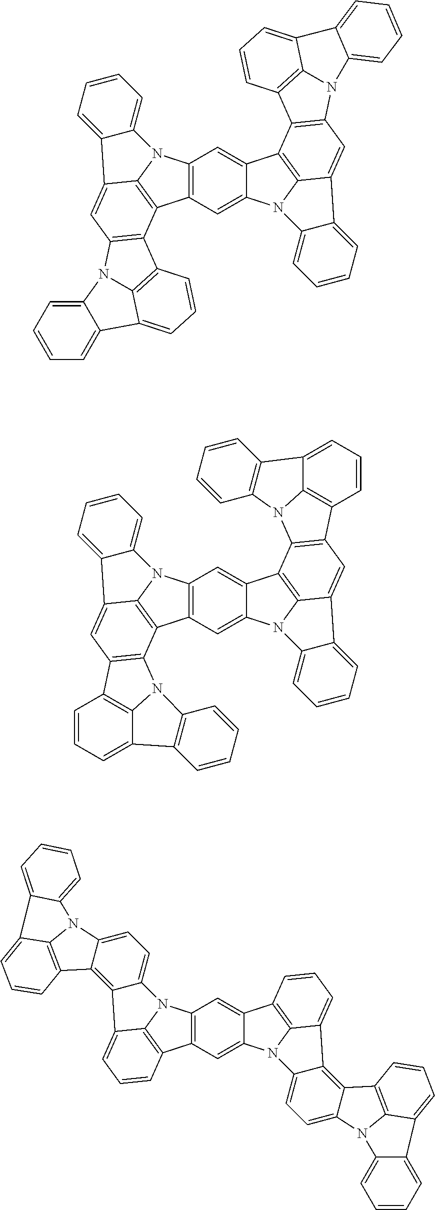 [SMILES: c1ccc2c(c1)c1cccc3c4c5c6cc7c(cc6n6c8ccccc8c(cc4n2c13)c56)c1c2c3cccc4c5ccccc5n(c2cc2c5ccccc5n7c21)c43 ; c1ccc2c(c1)c1cc3c4cccc5c6ccccc6n(c54)c3c3c4cc5c(cc4n2c13)c1c2c(cc3c4cccc6c7ccccc7n(c64)c31)c1ccccc1n52 ; c1ccc2c(c1)c1cccc3c4c5c6cccc7c8cc9c(cc8n(c5ccc4n2c13)c76)c1cccc2c3c4c5cccc6c7ccccc7n(c4ccc3n9c12)c65]

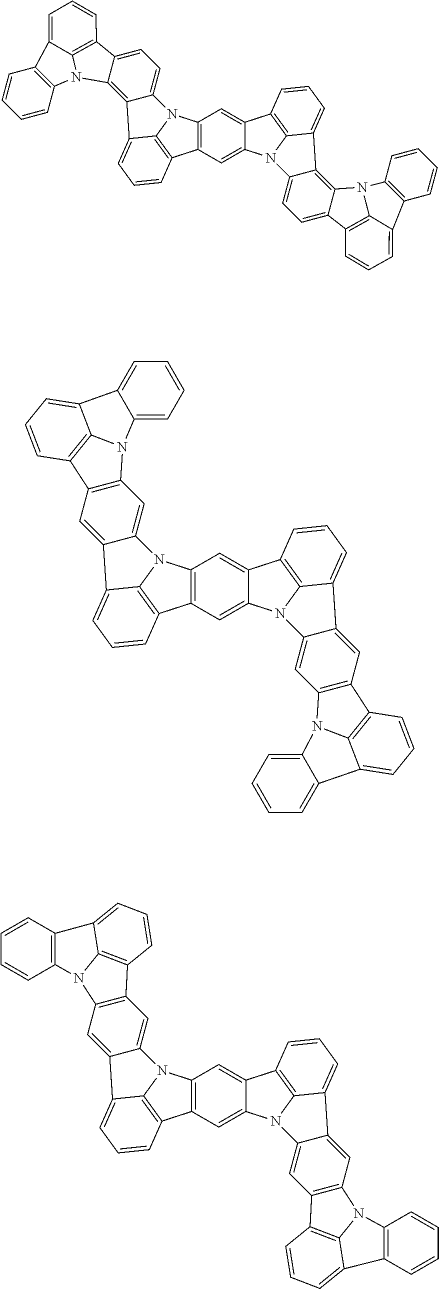 [SMILES: c1ccc2c(c1)c1cccc3c4ccc5c(c6cccc7c8cc9c(cc8n5c76)c5cccc6c7c(ccc8c%10cccc%11c%12ccccc%12n(c%11%10)c87)n9c56)c4n2c13 ; c1ccc2c(c1)c1cccc3c4cc5c6cccc7c8cc9c(cc8n(c5cc4n2c13)c76)c1cccc2c3cc4c5cccc6c7ccccc7n(c4cc3n9c12)c65 ; c1ccc2c(c1)c1cccc3c4cc5c(cc4n2c13)c1cccc2c3cc4c(cc3n5c12)c1cccc2c3cc5c(cc3n4c21)c1cccc2c3ccccc3n5c21]

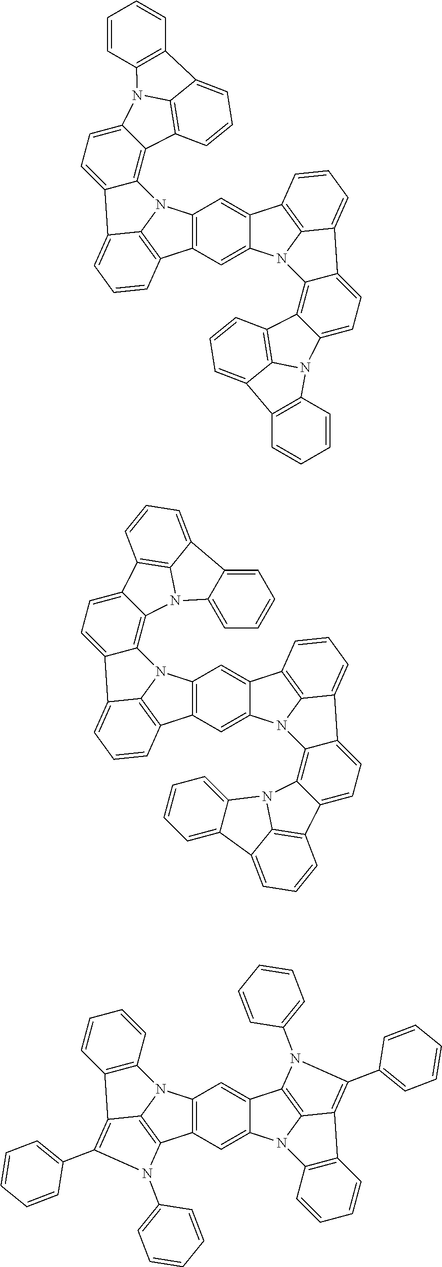 [SMILES: c1ccc2c(c1)c1cccc3c4c(ccc5c6cccc7c8cc9c(cc8n(c76)c54)c4cccc5c6ccc7c(c8cccc%10c%11ccccc%11n7c%108)c6n9c45)n2c13 ; c1ccc2c(c1)c1cccc3c4ccc5c6cccc7c8cc9c(cc8n(c76)c5c4n2c13)c1cccc2c3ccc4c5cccc6c7ccccc7n(c65)c4c3n9c12 ; c1ccc(-c2c3c4ccccc4n4c5cc6c7c8c(c(-c9ccccc9)n7-c7ccccc7)c7ccccc7n8c6cc5c(c34)n2-c2ccccc2)cc1]

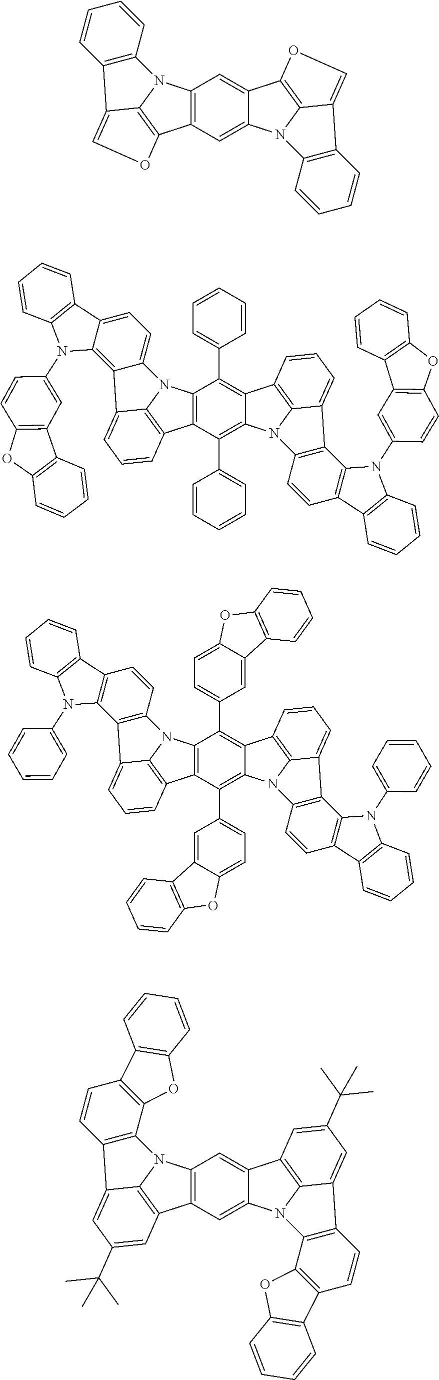 [SMILES: c1ccc2c(c1)c1coc3c4cc5c(cc4n2c13)c1occ2c3ccccc3n5c21 ; c1ccc(-c2c3c4cccc5c6c7c(ccc6n(c3c(-c3ccccc3)c3c6cccc8c9c%10c(ccc9n(c23)c68)c2ccccc2n%10-c2ccc3oc6ccccc6c3c2)c45)c2ccccc2n7-c2ccc3oc4ccccc4c3c2)cc1 ; c1ccc(-n2c3ccccc3c3ccc4c(c5cccc6c7c(-c8ccc9oc%10ccccc%10c9c8)c8c(c(-c9ccc%10oc%11ccccc%11c%10c9)c7n4c65)c4cccc5c6c7c(ccc6n8c45)c4ccccc4n7-c4ccccc4)c32)cc1 ; CC(C)(C)c1cc2c3cc4c(cc3n3c2c(c1)c1ccc2c5ccccc5oc2c13)c1cc(C(C)(C)C)cc2c3ccc5c6ccccc6oc5c3n4c12]

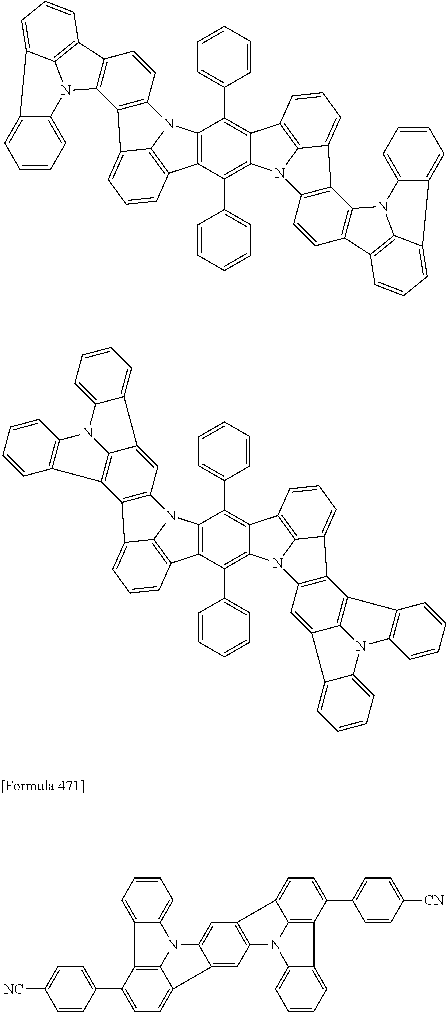 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; c1ccc(-c2c3c4cccc5c6c7c8ccccc8n8c9ccccc9c(cc6n(c3c(-c3ccccc3)c3c6cccc9c%10c%11c%12ccccc%12n%12c%13ccccc%13c(cc%10n(c23)c69)c%11%12)c45)c78)cc1 ; N#Cc1ccc(-c2ccc3c4cc5c(cc4n4c6ccccc6c2c34)c2ccc(-c3ccc(C#N)cc3)c3c4ccccc4n5c23)cc1]

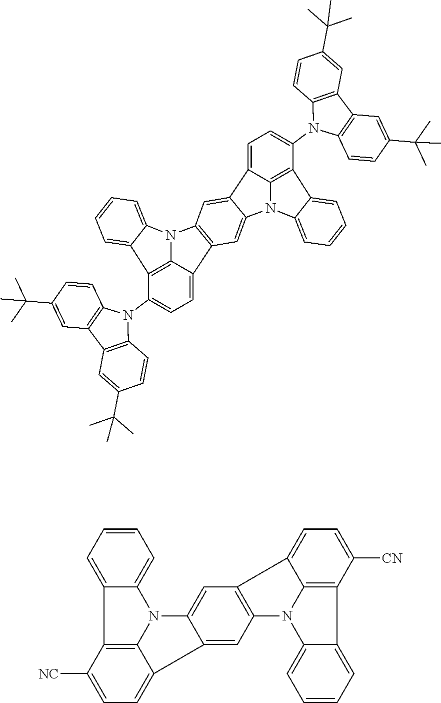 [SMILES: CC(C)(C)c1ccc2c(c1)c1cc(C(C)(C)C)ccc1n2-c1ccc2c3cc4c(cc3n3c5ccccc5c1c23)c1ccc(-n2c3ccc(C(C)(C)C)cc3c3cc(C(C)(C)C)ccc32)c2c3ccccc3n4c12 ; N#Cc1ccc2c3cc4c(cc3n3c5ccccc5c1c23)c1ccc(C#N)c2c3ccccc3n4c12]

### Compound Represented by Formula (7)

The compound represented by the formula (7) will be described below.

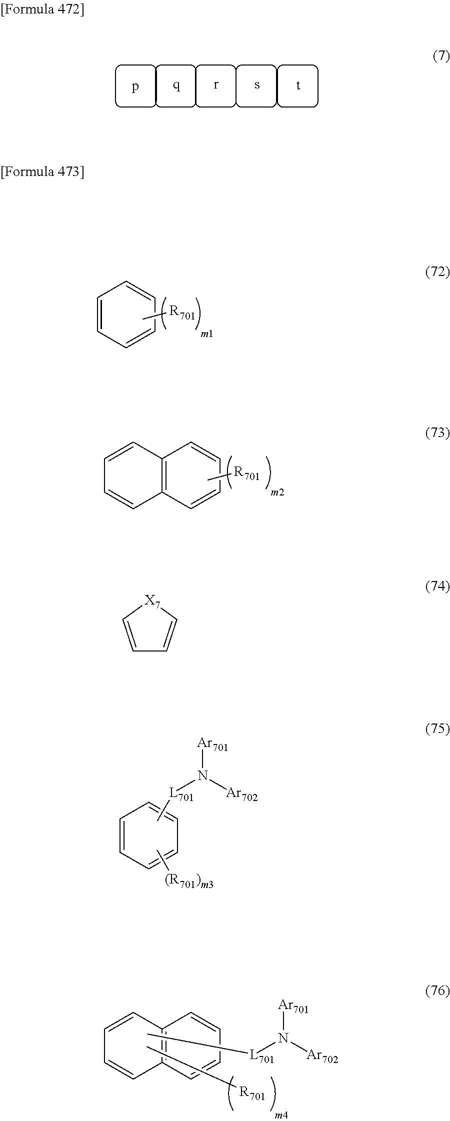

In the formula (7):
a ring r is a ring represented by the formula (72) or the formula (73), the ring r being fused with adjacent ring(s) at any position(s);
a ring q and a ring s are each independently a ring represented by the formula (74) and fused with adjacent ring(s) at any position(s);
a ring p and a ring t are each independently a structure represented by the formula (75) or the formula (76) and fused with adjacent ring(s) at any position(s);
X₇ is an oxygen atom, a sulfur atom, or NR₇₀₂;
when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded;
R₇₀ and R₇₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; and
L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;
m1 is 0, 1, or 2;
m2 is 0, 1, 2, 3, or 4;
m3 is each independently 0, 1, 2, or 3;
m4 is each independently 0, 1, 2, 3, 4, or 5;
when a plurality of R₇₀₁ are present, the plurality of R₇₀₁ are mutually the same or different;
when a plurality of X₇ are present, the plurality of X₇ are mutually the same or different;
when a plurality of R₇₀₂ are present, the plurality of R₇₀₂ are mutually the same or different;
when a plurality of Ar₇₀₁ are present, the plurality of Ar₇₀₁ are mutually the same or different;
when a plurality of Ar₇₀₂ are present, the plurality of Ar₇₀₂ are mutually the same or different; and
when a plurality of L₇₀₁ are present, the plurality of L₇₀₁ are mutually the same or different.

In the formula (7), each of the ring p, ring q, ring r, ring s, and ring t is fused with an adjacent ring(s) sharing two carbon atoms. The fused position and orientation are not limited but may be defined as required.

In an exemplary embodiment, in the formula (72) or the formula (73) representing the ring r, m1=0 or m2=0 is satisfied.

In an exemplary embodiment, the compound represented by the formula (7) is represented by any one of formulae (71-1) to (71-6) below.

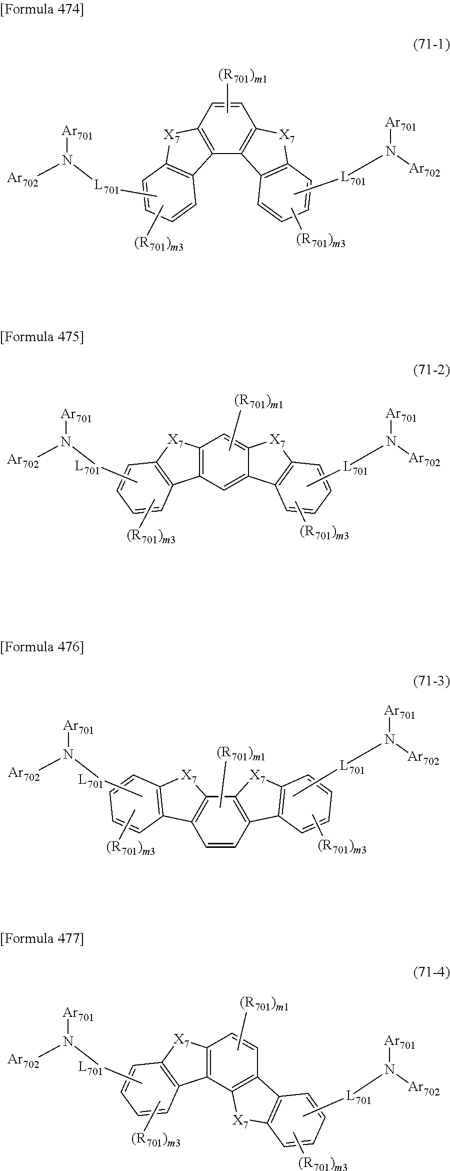

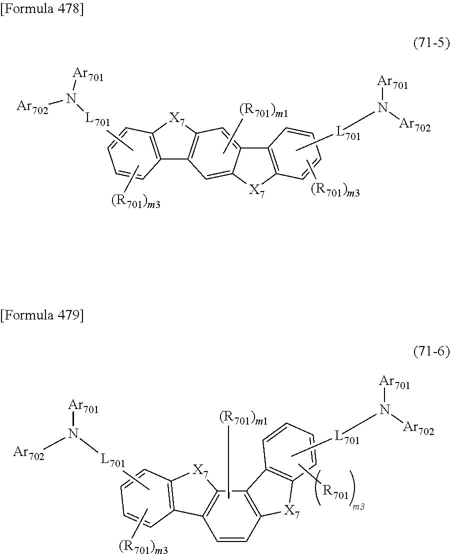

In the formulae (71-1) to (71-6), R₇₀₁, X₇, Ar₇₀₁, Ar₇₀₂, L₇₀₁, m1 and m3 respectively represent the same as R₇₀₁, X₇, Ar₇₀₁, Ar₇₀₂, L₇₀₁, m1 and m3 in the formula (7).

In an exemplary embodiment, the compound represented by the formula (7) is represented by any one of formulae (71-11) to (71-13) below.

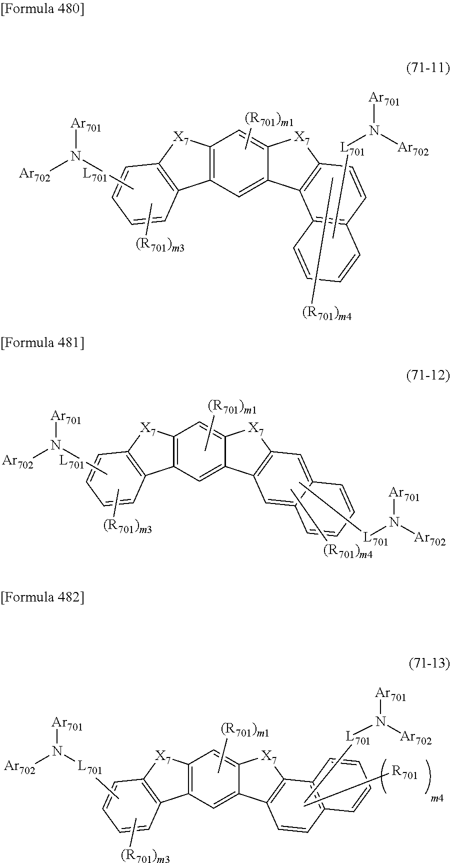

In the formulae (71-11) to (71-13), R₇₀₁, X₇, Ar₇₀₁, Ar₇₀₂, L₇₀₁, m1, m3 and m4 respectively represent the same as R₇₀₁, X₇, Ar₇₀₁, Ar₇₀₂, L₇₀₁, m1, m3 and m4 in the formula (7).

In an exemplary embodiment, the compound represented by the formula (7) is represented by any one of formulae (71-21) to (71-25) below.

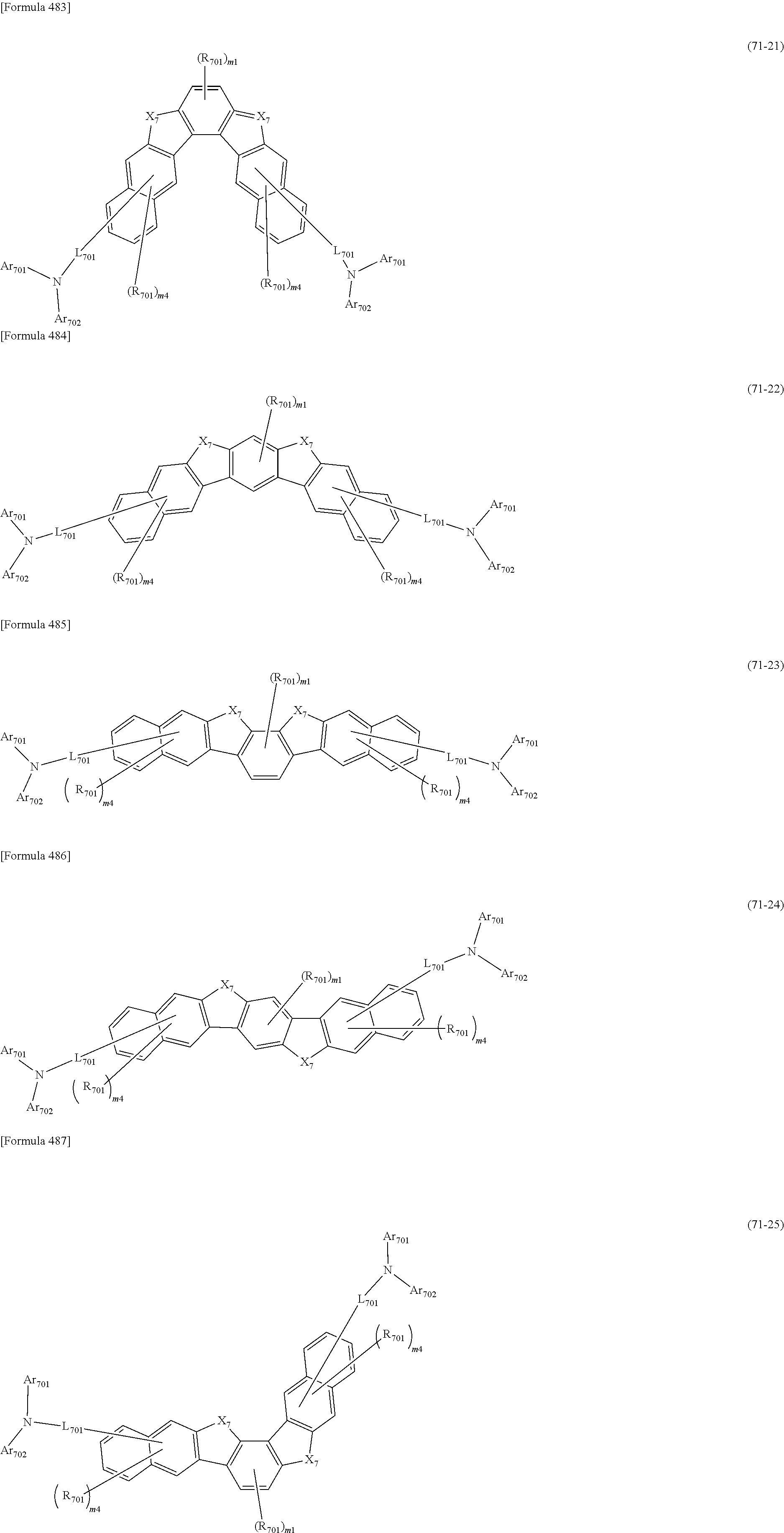

In the formulae (71-21) to (71-25), R₇₀₁, X₇, Ar₇₀₁, Ar₇₀₂, L₇₀₁, m1 and m4 respectively represent the same as R₇₀₁, X₇, Ar₇₀₁, Ar₇₀₂, L₇₀₁, m1 and m4 in the formula (7).

In an exemplary embodiment, the compound represented by the formula (7) is represented by any one of formulae (71-31) to (71-33) below.

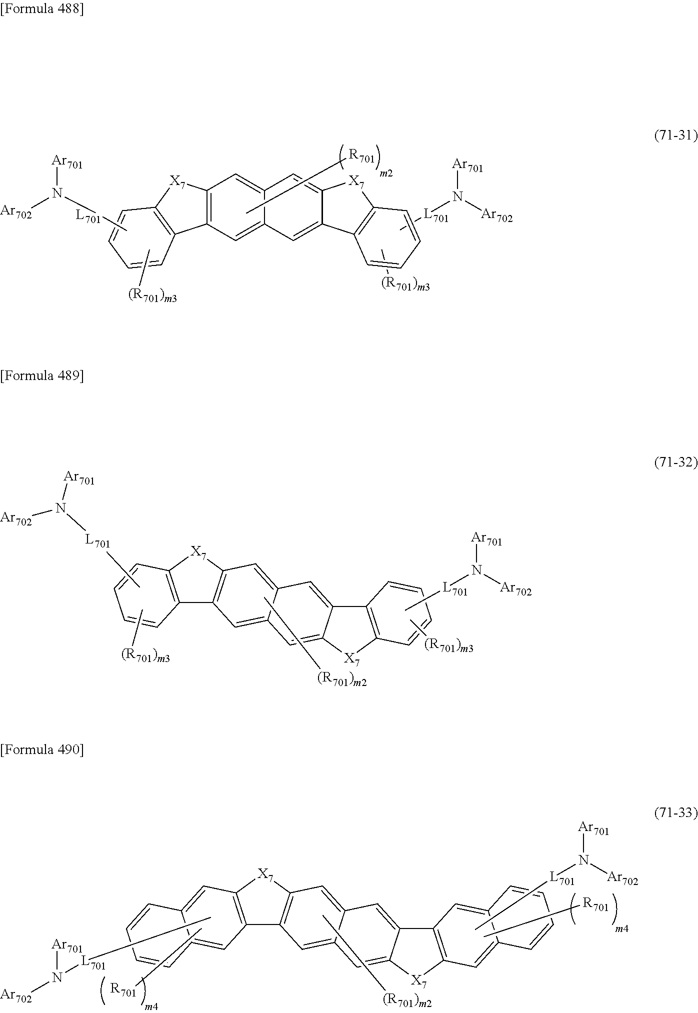

In the formulae (71-31) to (71-33), R₇₀₁, X₇, Ar₇₀₁, Ar₇₀₂, L₇₀₁, and m2 to m4 respectively represent the same as R₇₀₁, X₇, Ar₇₀₁, Ar₇₀₂, L₇₀₁, and m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and Ar₇₀₂ is a substituted or unsubstituted heterocyclic group having 5 to 50 ring atoms.

### Specific Examples of Compound Represented by Formula (7)

Specific examples of the compound represented by the formula (7) include compounds as below.

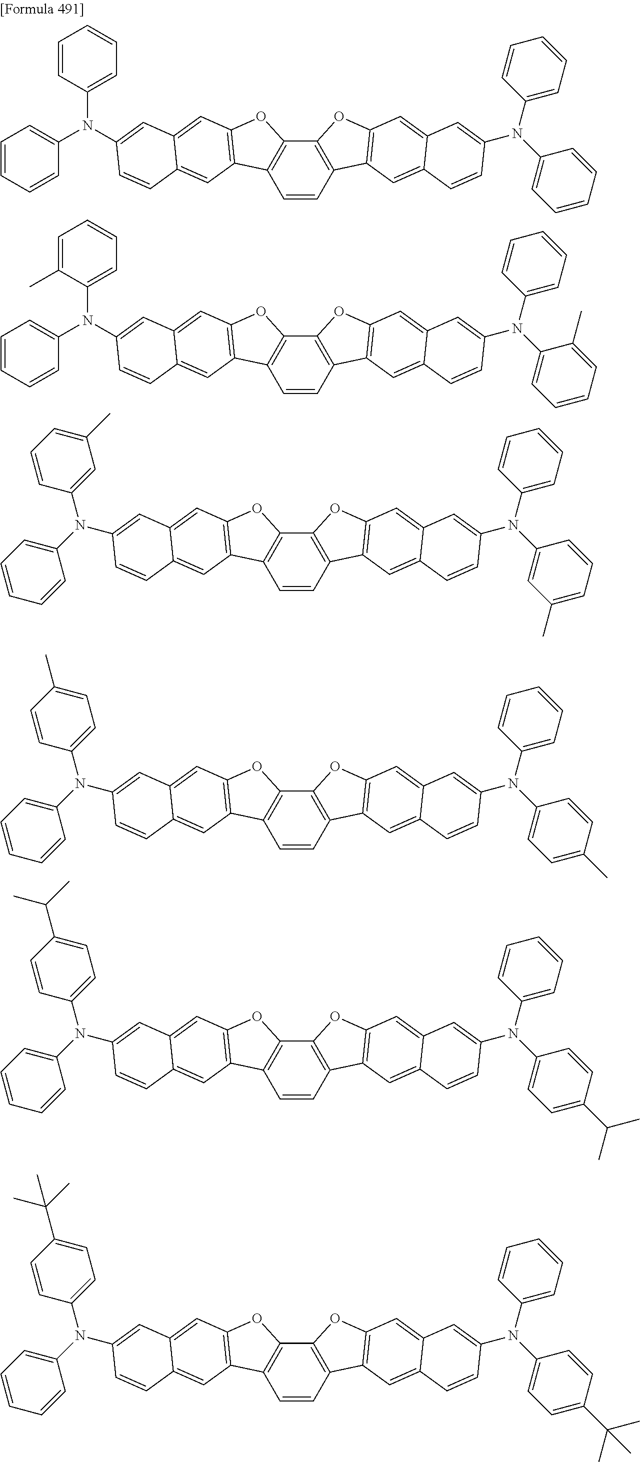

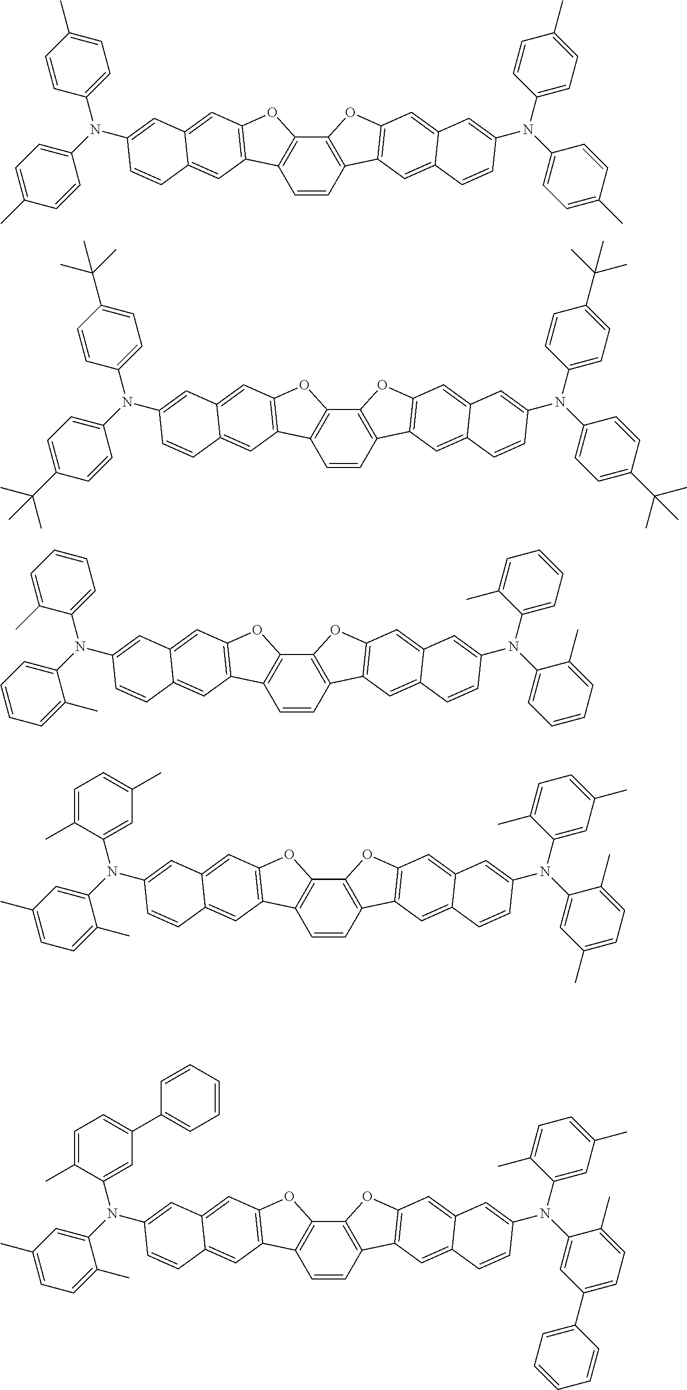

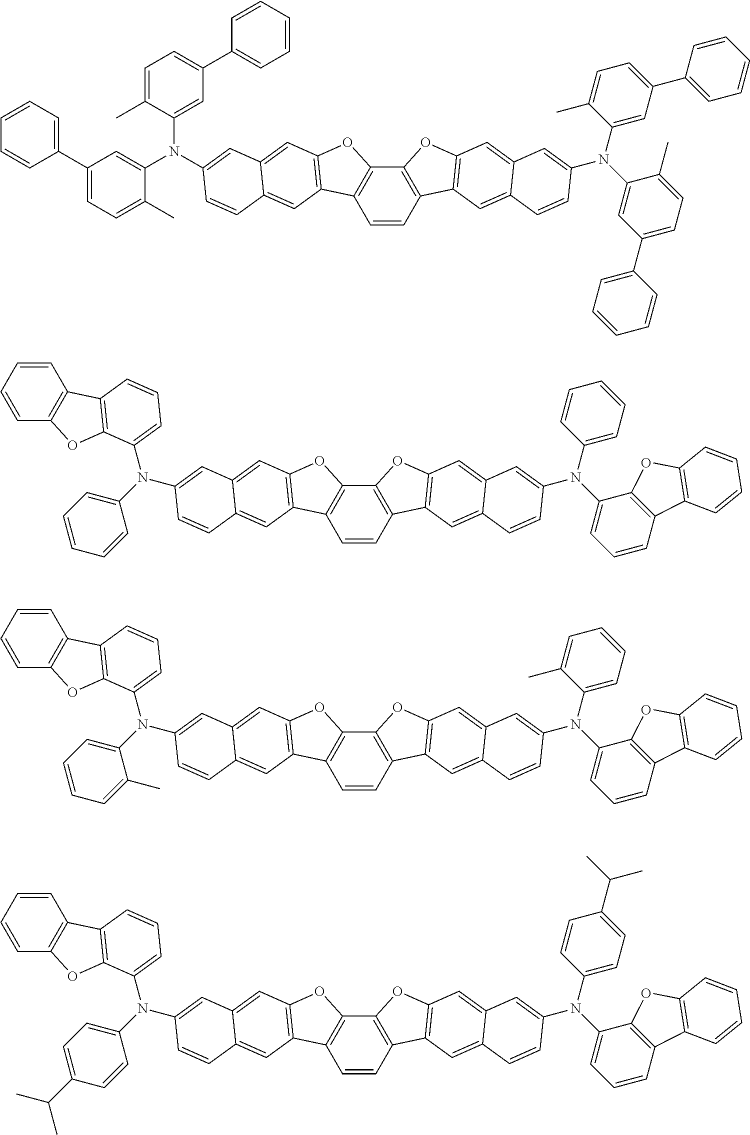 [SMILES: Cc1ccc(-c2ccccc2)cc1N(c1ccc2cc3c(cc2c1)oc1c3ccc2c3cc4ccc(N(c5cc(-c6ccccc6)ccc5C)c5cc(-c6ccccc6)ccc5C)cc4cc3oc21)c1cc(-c2ccccc2)ccc1C ; c1ccc(N(c2ccc3cc4c(cc3c2)oc2c4ccc3c4cc5ccc(N(c6ccccc6)c6cccc7c6oc6ccccc67)cc5cc4oc32)c2cccc3c2oc2ccccc23)cc1 ; Cc1ccccc1N(c1ccc2cc3c(cc2c1)oc1c3ccc2c3cc4ccc(N(c5ccccc5C)c5cccc6c5oc5ccccc56)cc4cc3oc21)c1cccc2c1oc1ccccc12 ; CC(C)c1ccc(N(c2ccc3cc4c(cc3c2)oc2c4ccc3c4cc5ccc(N(c6ccc(C(C)C)cc6)c6cccc7c6oc6ccccc67)cc5cc4oc32)c2cccc3c2oc2ccccc23)cc1]

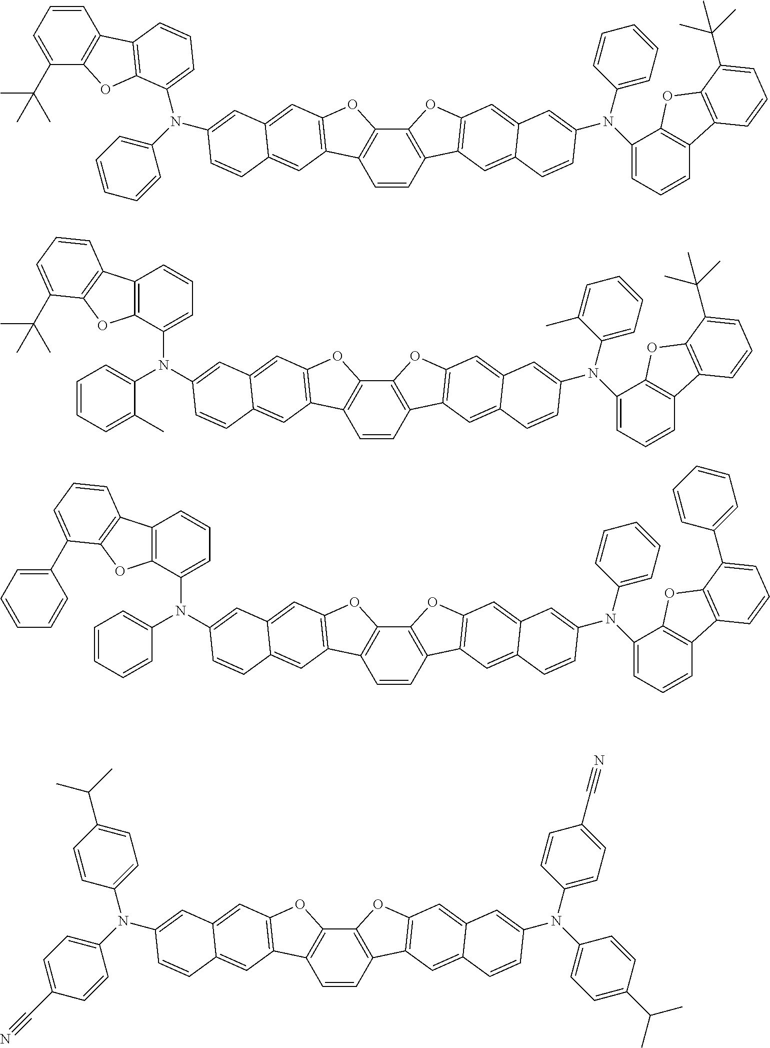

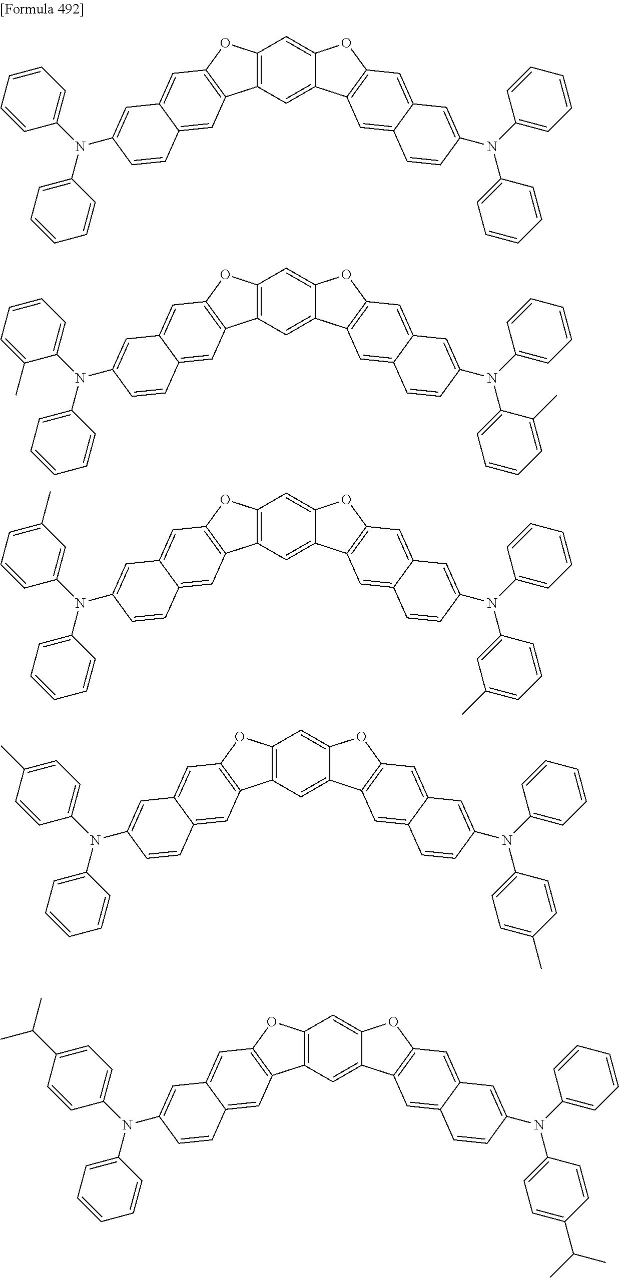

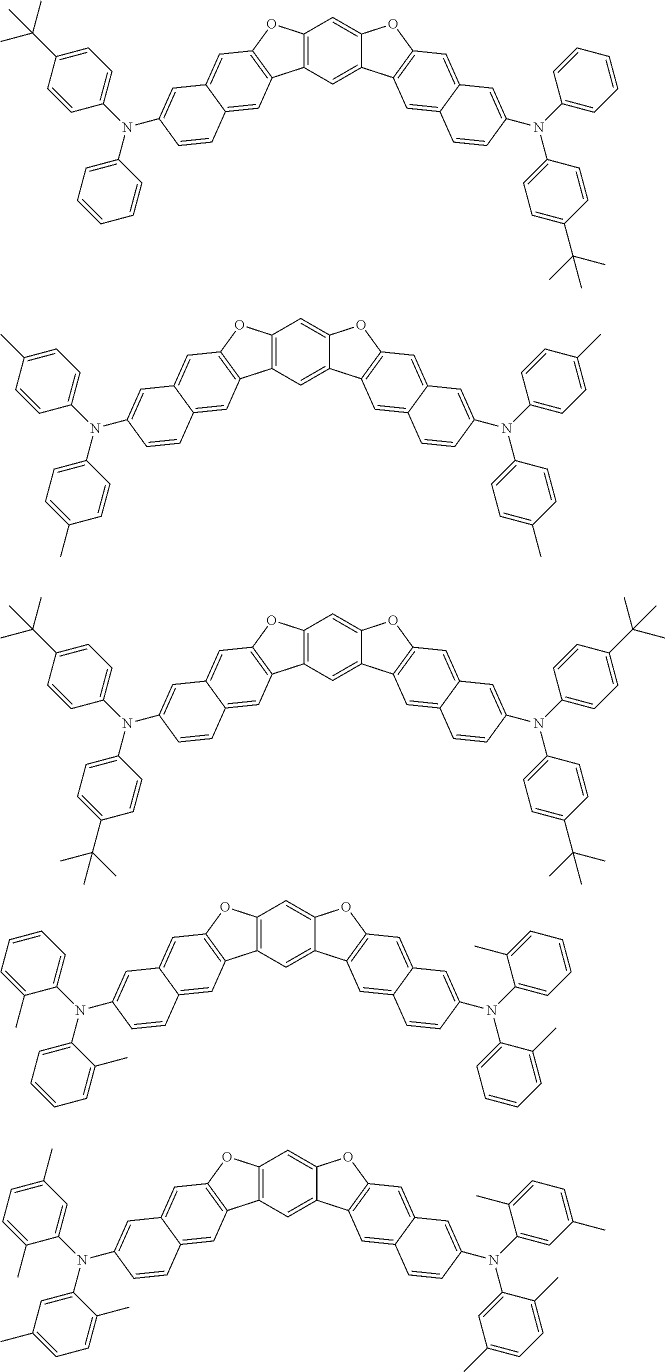

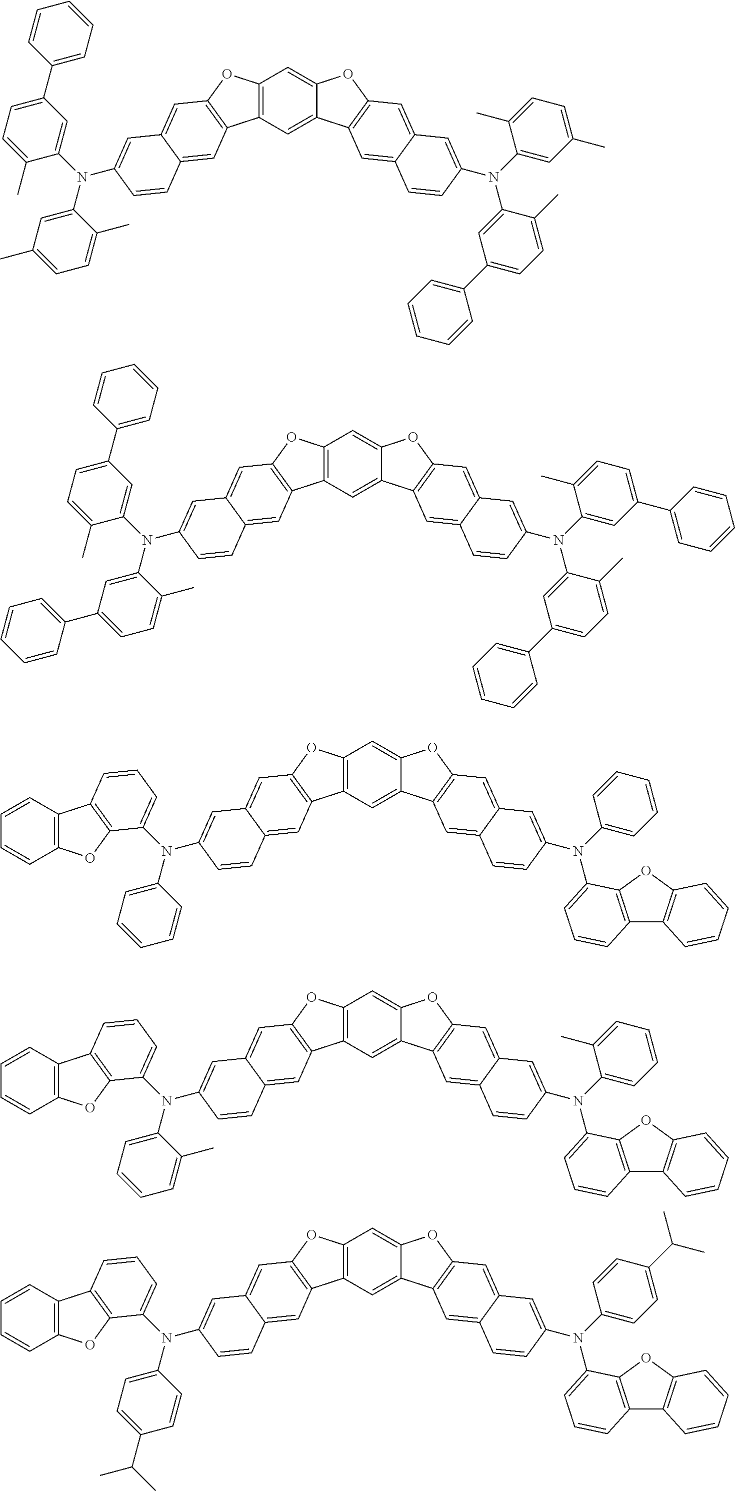

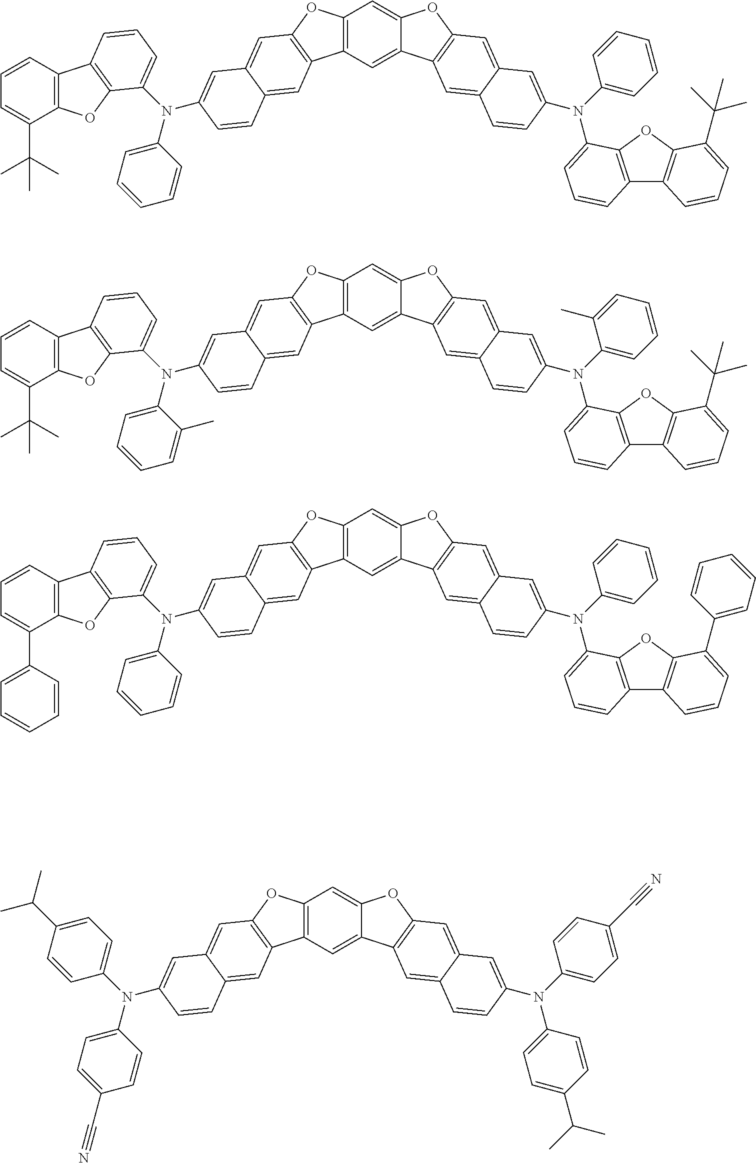

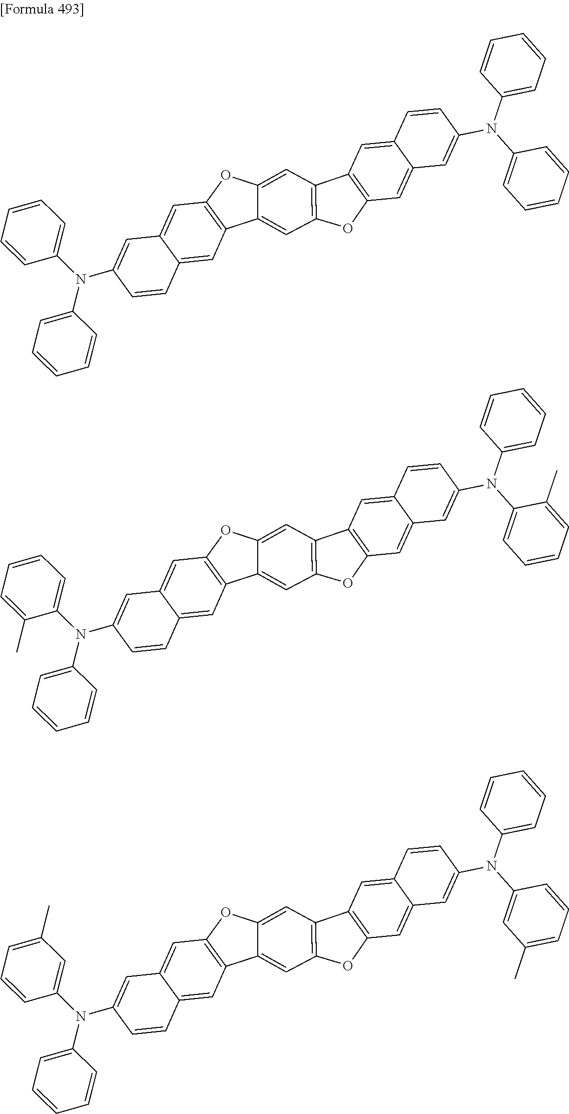 [SMILES: c1ccc(N(c2ccccc2)c2ccc3cc4c(cc3c2)oc2cc3c(cc24)oc2cc4cc(N(c5ccccc5)c5ccccc5)ccc4cc23)cc1 ; Cc1ccccc1N(c1ccccc1)c1ccc2cc3c(cc2c1)oc1cc2c(cc13)oc1cc3cc(N(c4ccccc4)c4ccccc4C)ccc3cc12 ; Cc1cccc(N(c2ccccc2)c2ccc3cc4c(cc3c2)oc2cc3c(cc24)oc2cc4cc(N(c5ccccc5)c5cccc(C)c5)ccc4cc23)c1]

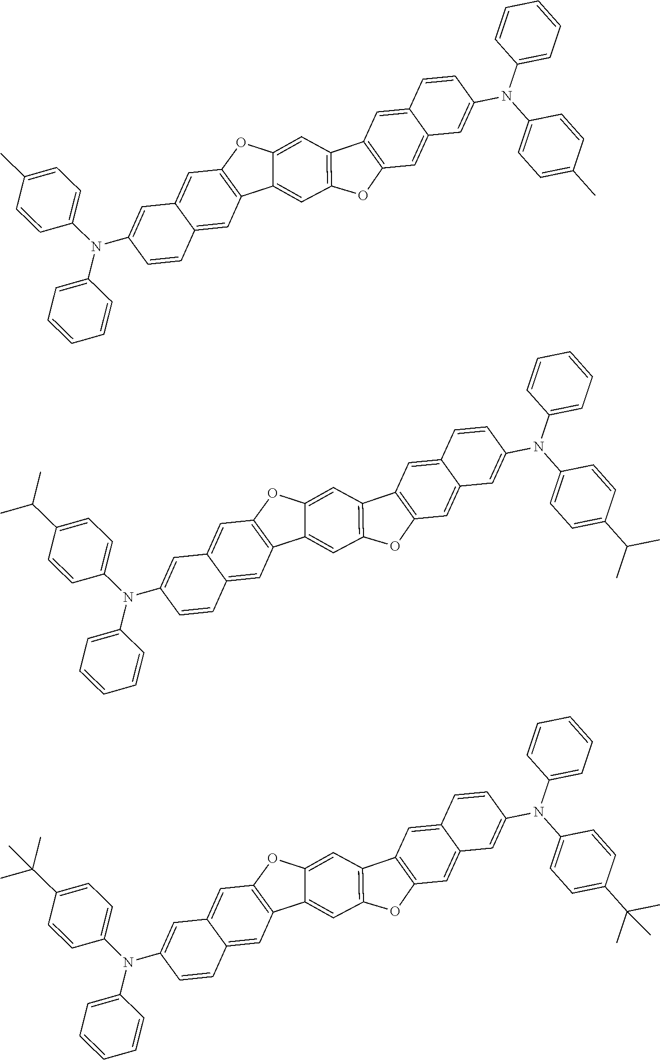

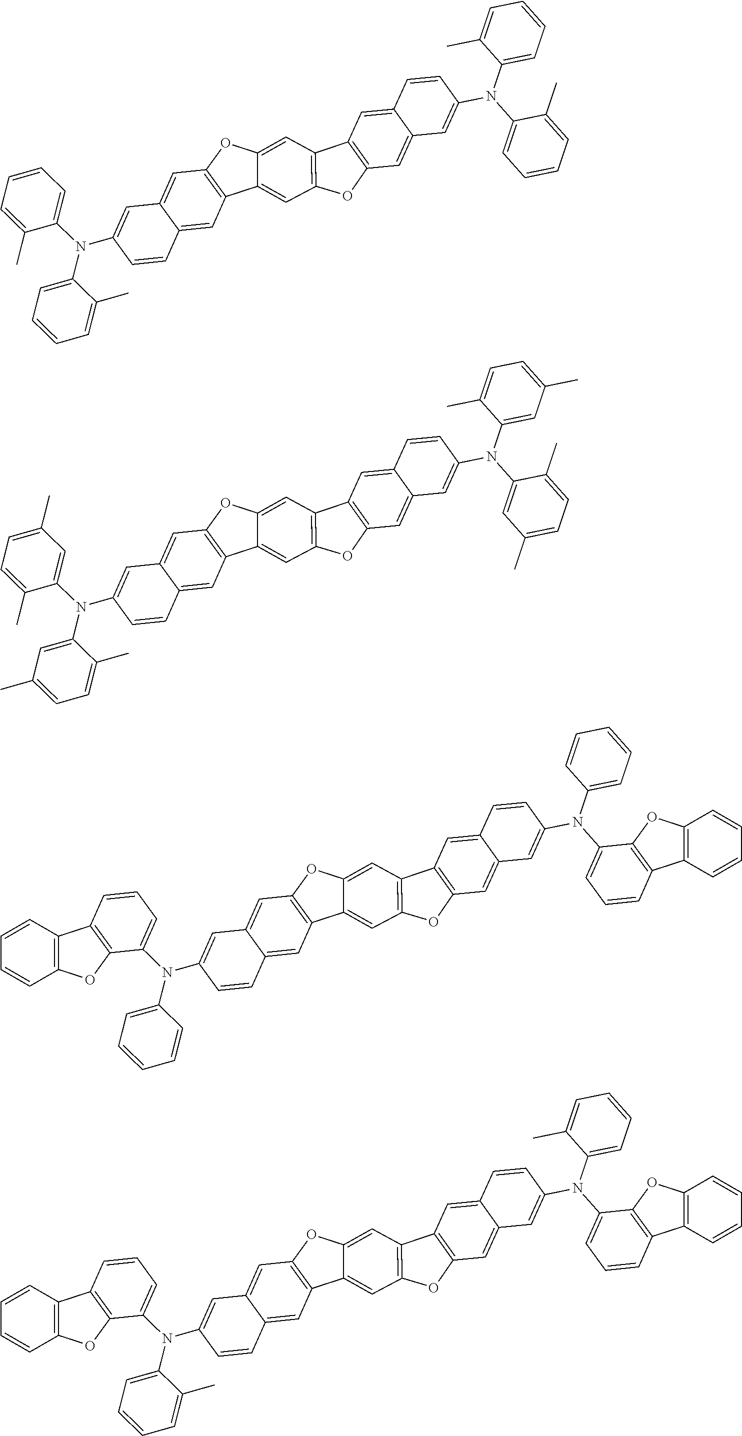

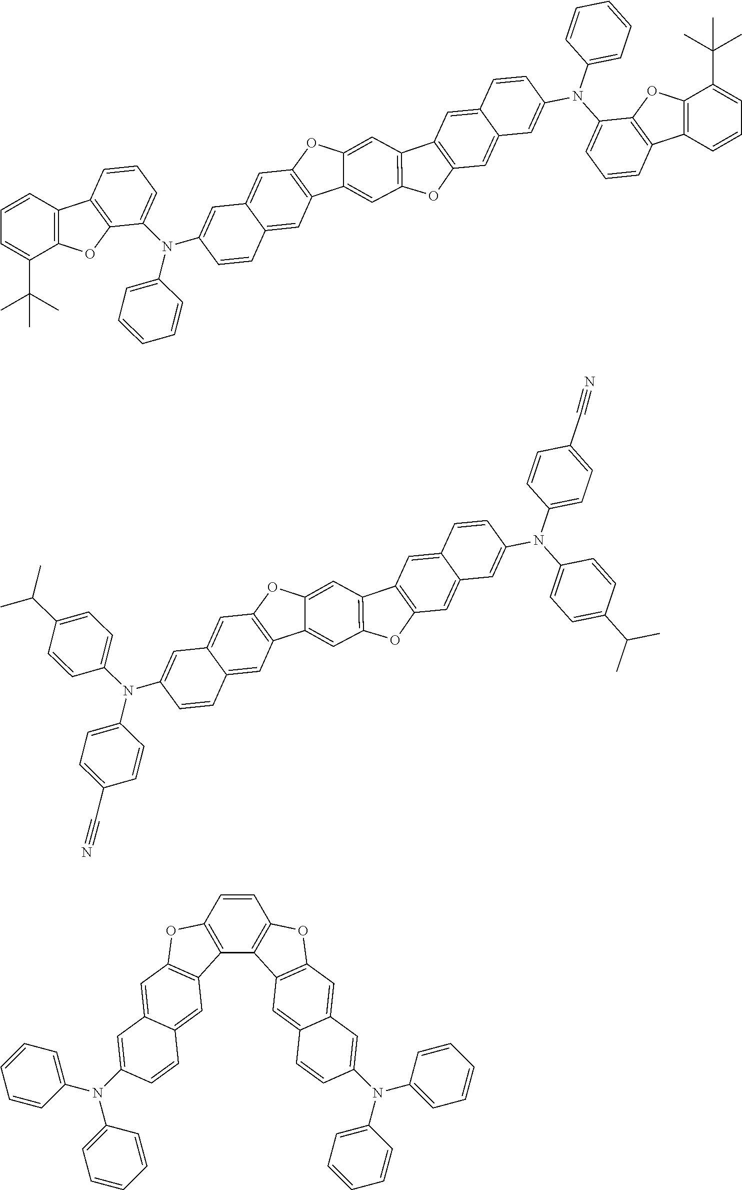

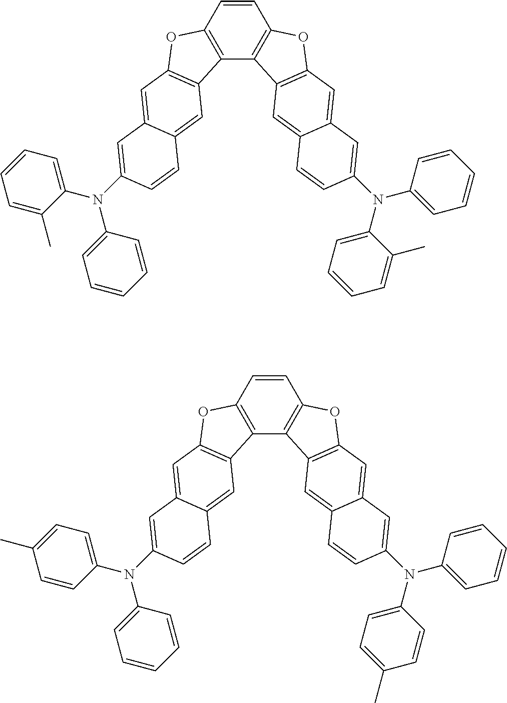 [SMILES: Cc1ccccc1N(c1ccccc1)c1ccc2cc3c(cc2c1)oc1ccc2oc4cc5cc(N(c6ccccc6)c6ccccc6C)ccc5cc4c2c13 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1]

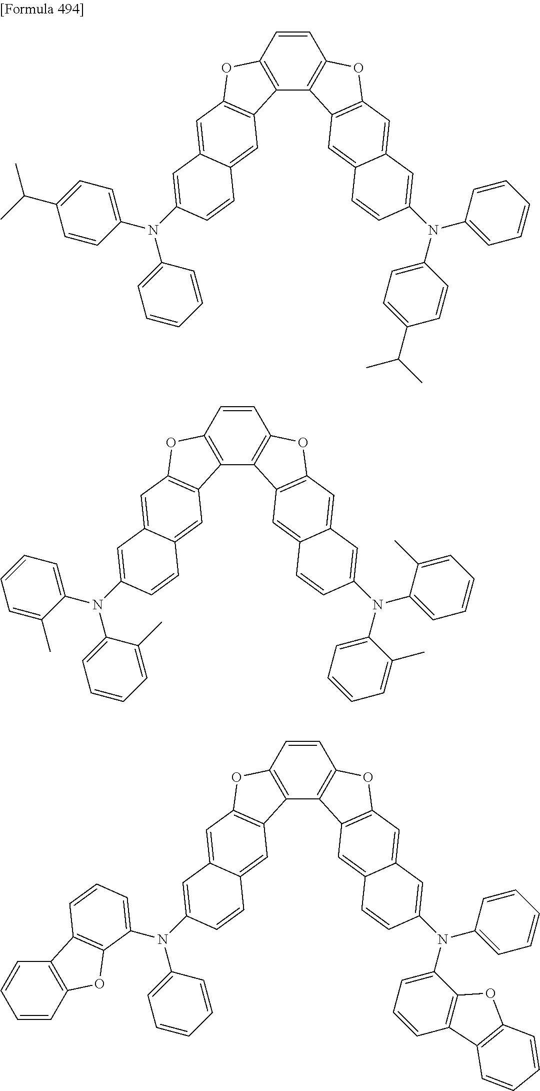 [SMILES: CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccccc1N(c1ccc2cc3c(cc2c1)oc1ccc2oc4cc5cc(N(c6ccccc6C)c6ccccc6C)ccc5cc4c2c13)c1ccccc1C ; c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1]

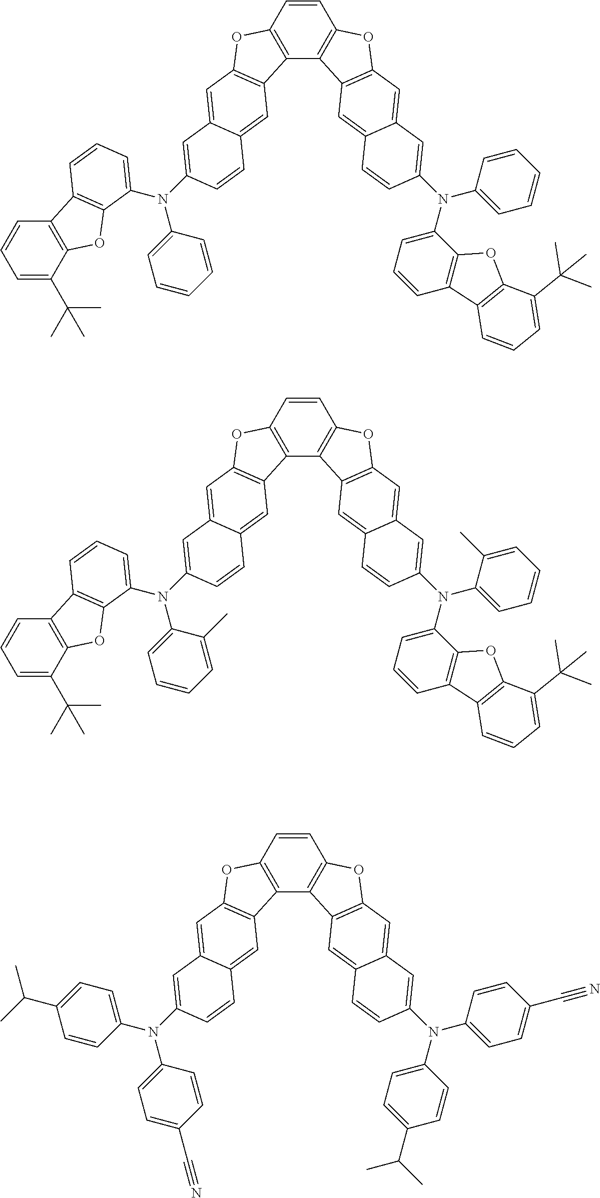

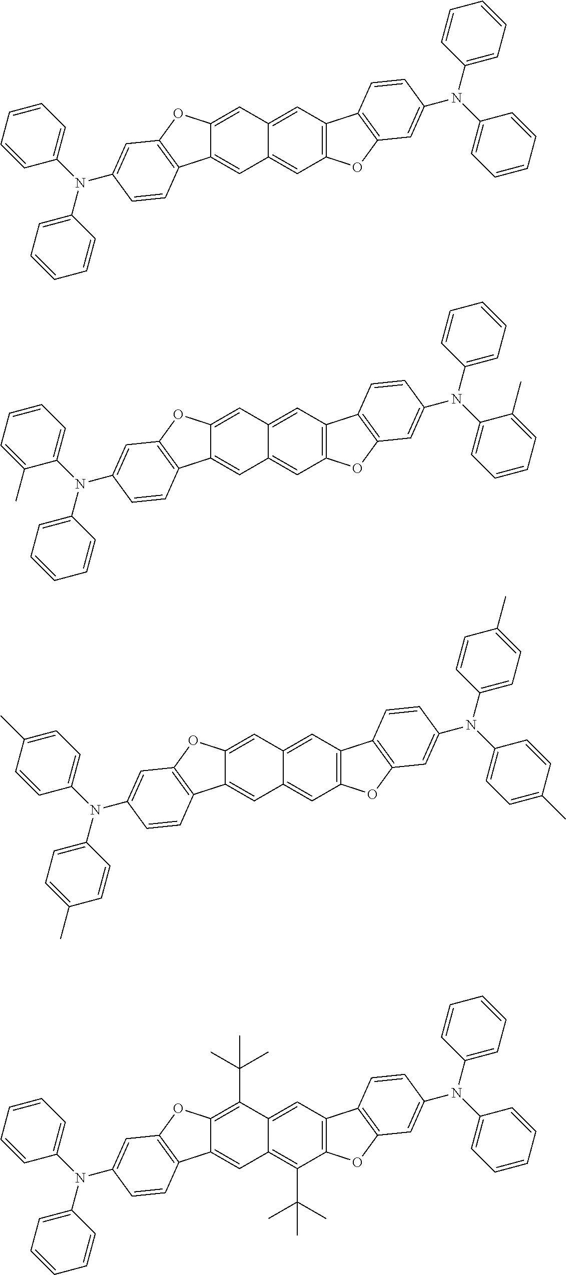

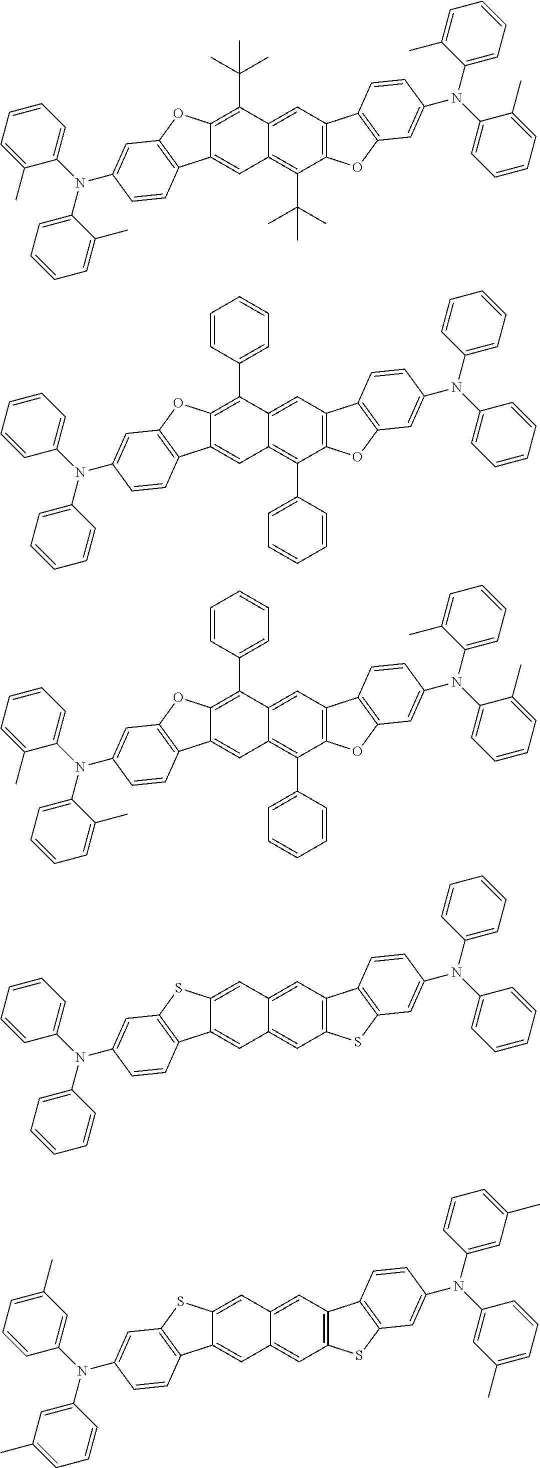

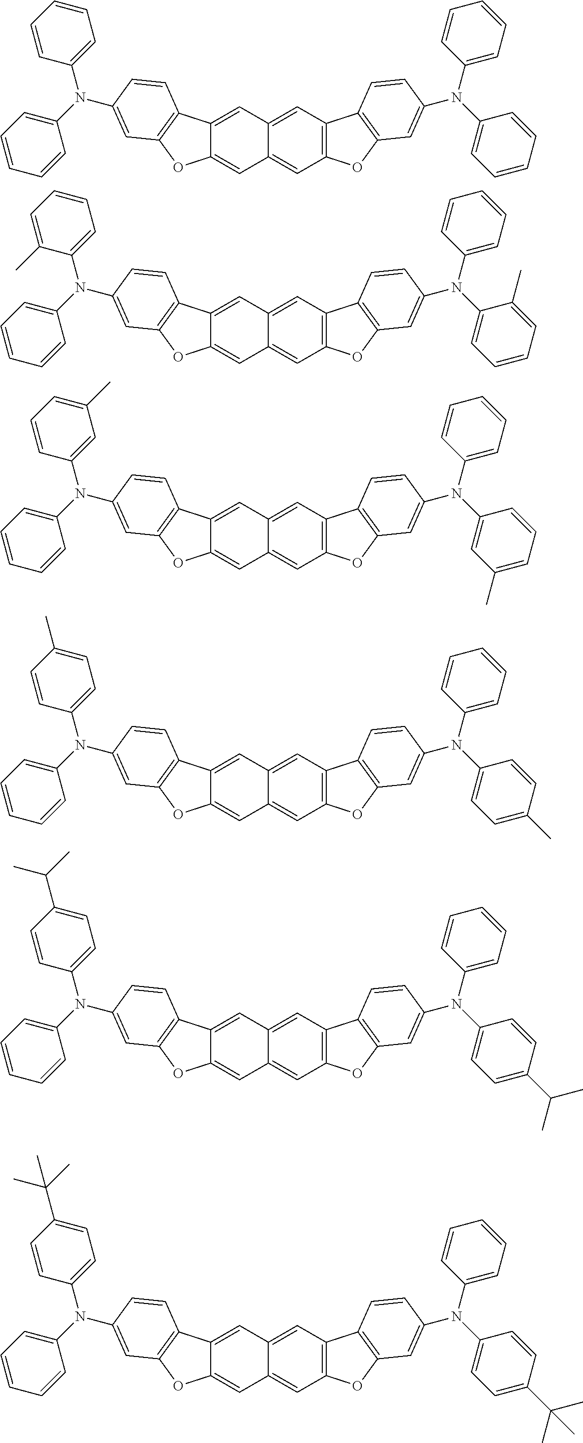

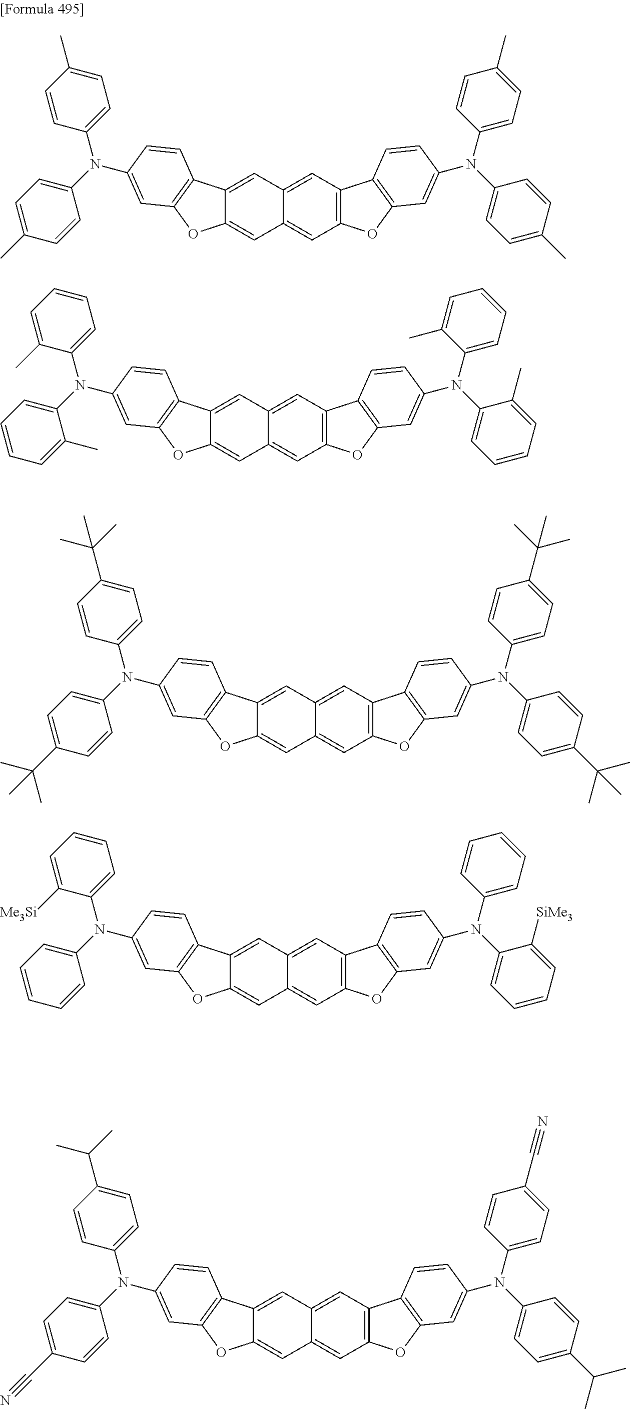

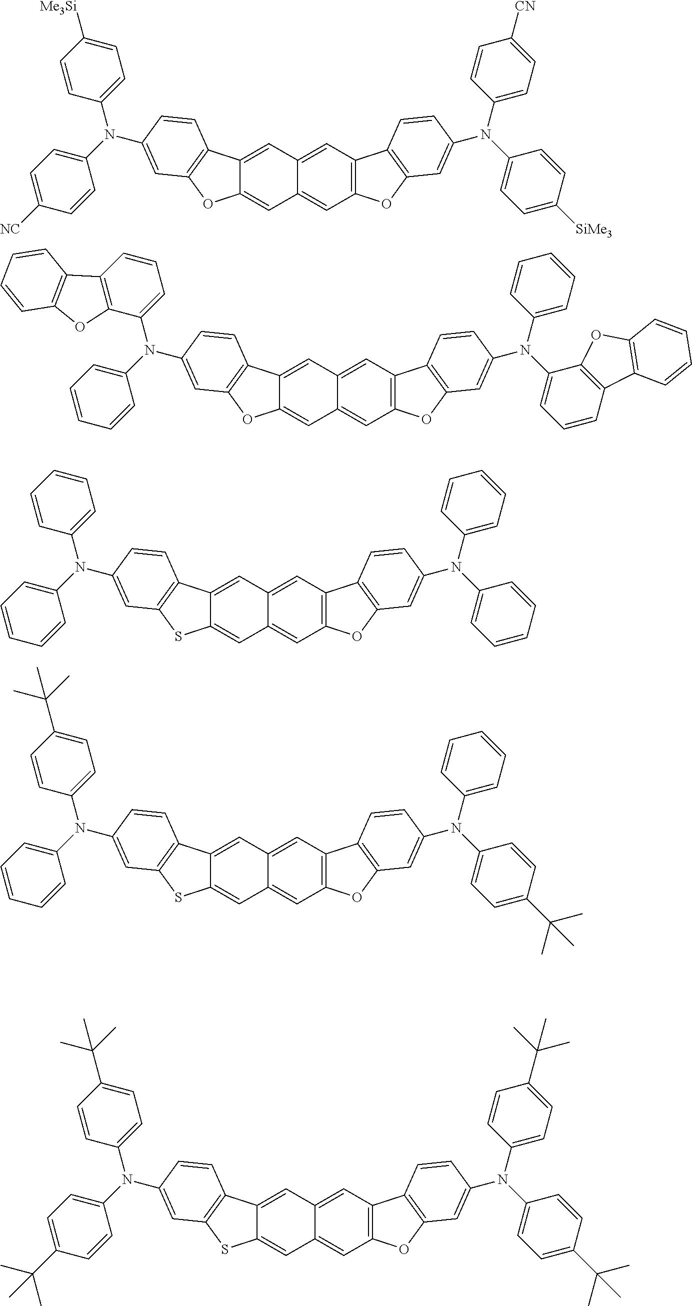

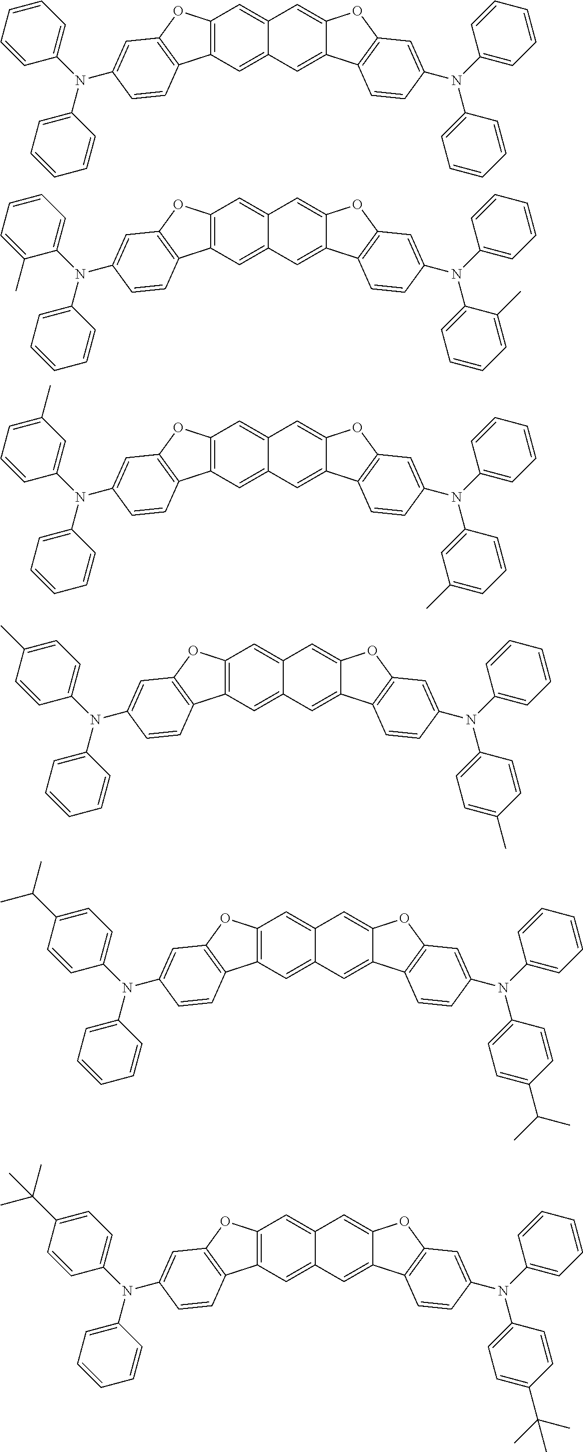

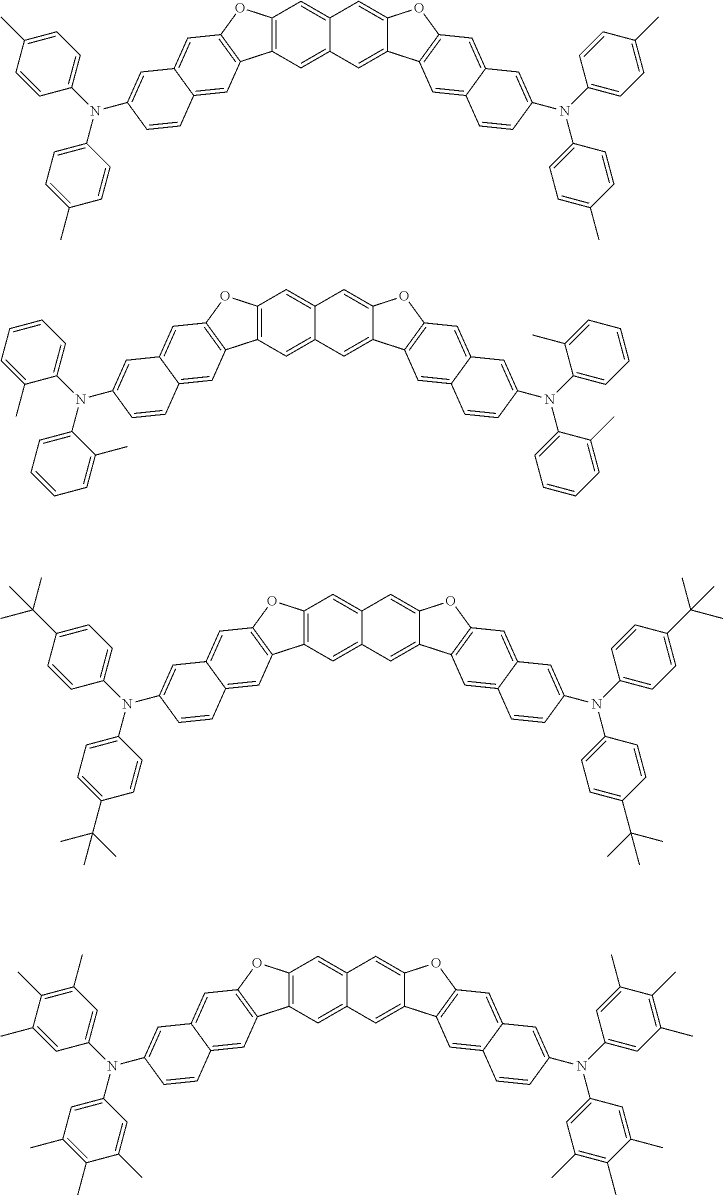

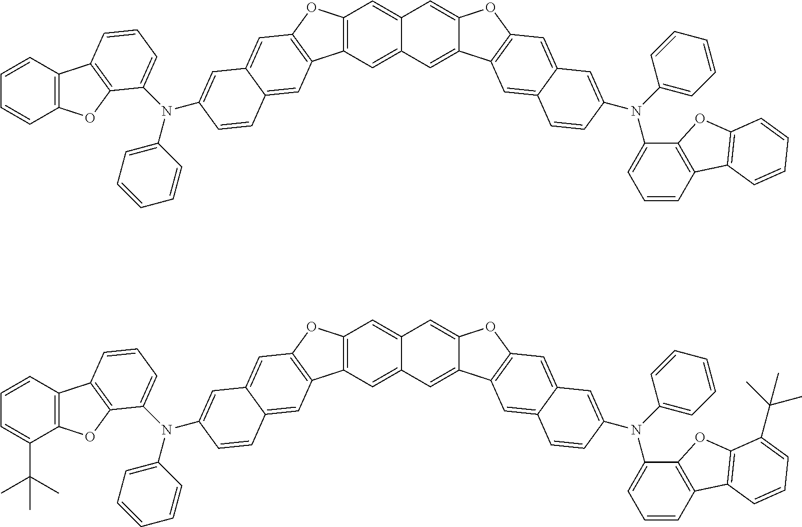 [SMILES: c1ccc(N(c2ccc3cc4c(cc3c2)oc2cc3cc5oc6cc7cc(N(c8ccccc8)c8cccc9c8oc8ccccc89)ccc7cc6c5cc3cc24)c2cccc3c2oc2ccccc23)cc1 ; CC(C)(C)c1cccc2c1oc1c(N(c3ccccc3)c3ccc4cc5c(cc4c3)oc3cc4cc6oc7cc8cc(N(c9ccccc9)c9cccc%10c9oc9c(C(C)(C)C)cccc9%10)ccc8cc7c6cc4cc35)cccc12]

### Compound Represented by Formula (8)

The compound represented by the formula (8) will be described below.

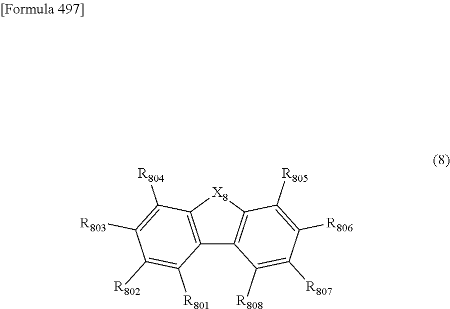

In the formula (8):
at least one combination of a combination of R₈₀₁ and R₈₀₂, a combination of R₈₀₂ and R₈₀₃, and a combination of R₈₀₃ and R₈₀₄ are mutually bonded to form a divalent group represented by a formula (82) below; and
at least one combination of a combination of R₈₀₅ and R₈₀₆, a combination of R₈₀₆ and R₈₀₇, and a combination of R₈₀₇ and R₈₀₈ are mutually bonded to form a divalent group represented by a formula (83) below.

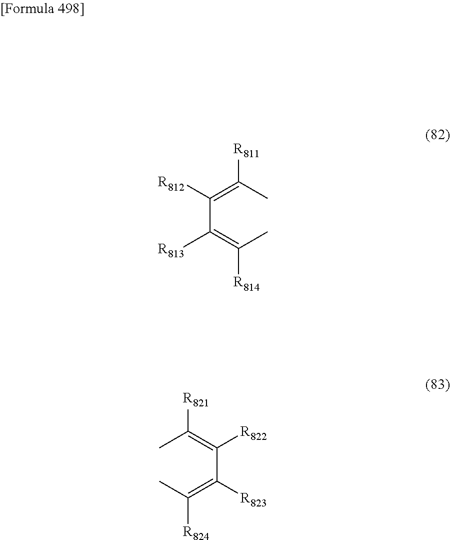

At least one of R₈₀₁ to R₈₀₄ not forming the divalent group represented by the formula (82) or R₈₁₁ to R₈₁₄ is a monovalent group represented by a formula (84) below;
at least one of R₈₀₅ to R₈₀₈ not forming the divalent group represented by the formula (83) or R₈₂₁ to R₈₂₄ is a monovalent group represented by a formula (84) below;
X₈ is an oxygen atom, a sulfur atom, or NR₈₀₉; and
R₈₀₁ to R₈₀₈ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

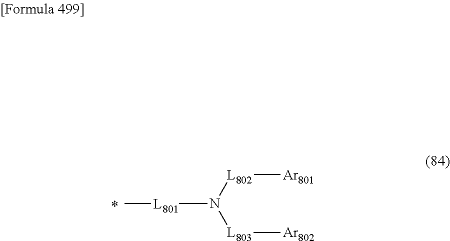

In the formula (84):
Ar₈₀₁ and Ar₈₀₂ are each independently a substituted or unsubstituted aryl group having 6 to 50 ring carbon atoms, or a substituted or unsubstituted heterocyclic group having 5 to 50 ring atoms;
L₈₀₁ to L₈₀₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (84) represents a bonding position to a cyclic structure represented by the formula (8) or a bonding position to a group represented by the formula (82) or (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, the compound represented by the formula (8) is represented by any one of formulae (81-1) to (81-6) below.

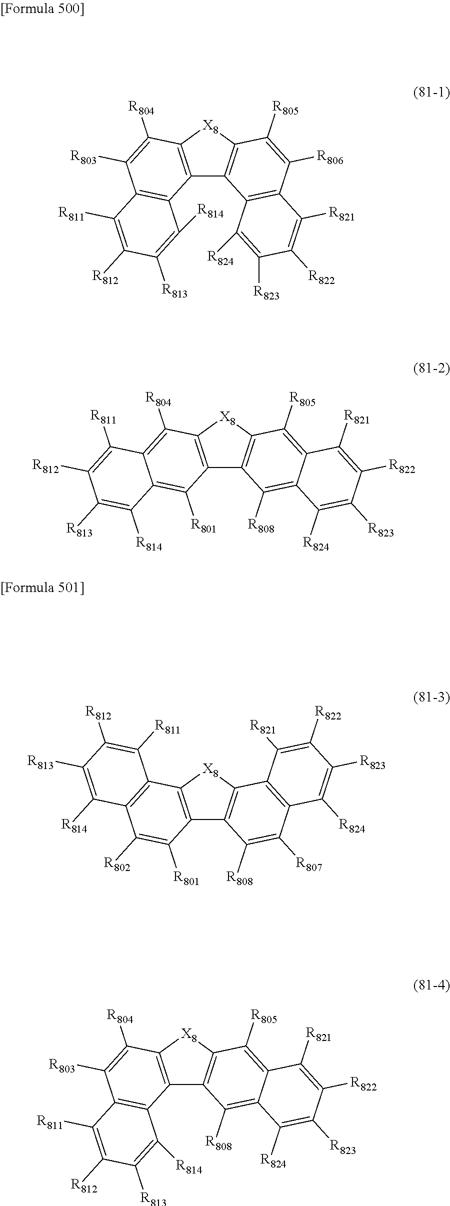

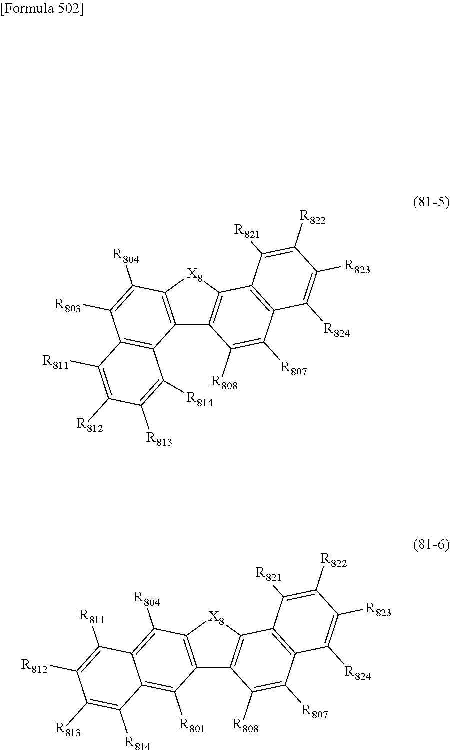

In the formulae (81-1) to (81-6):
X₈ represents the same as X₈ in the formula (8);
at least two of R₈₀₁ to R₈₂₄ are each a monovalent group represented by the formula (84); and
R₈₀₁ to R₈₂₄ not being the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (8) is represented by any one of formulae (81-7) to (81-18) below.

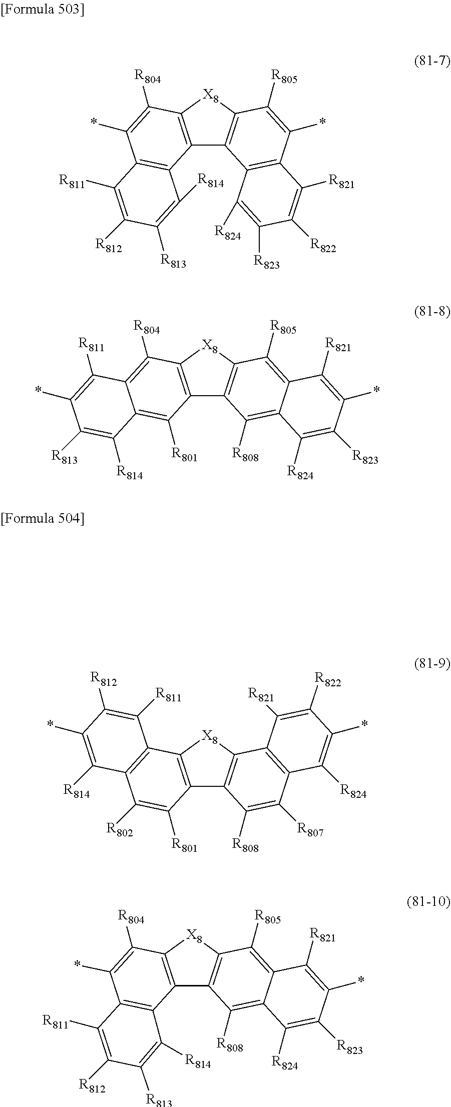

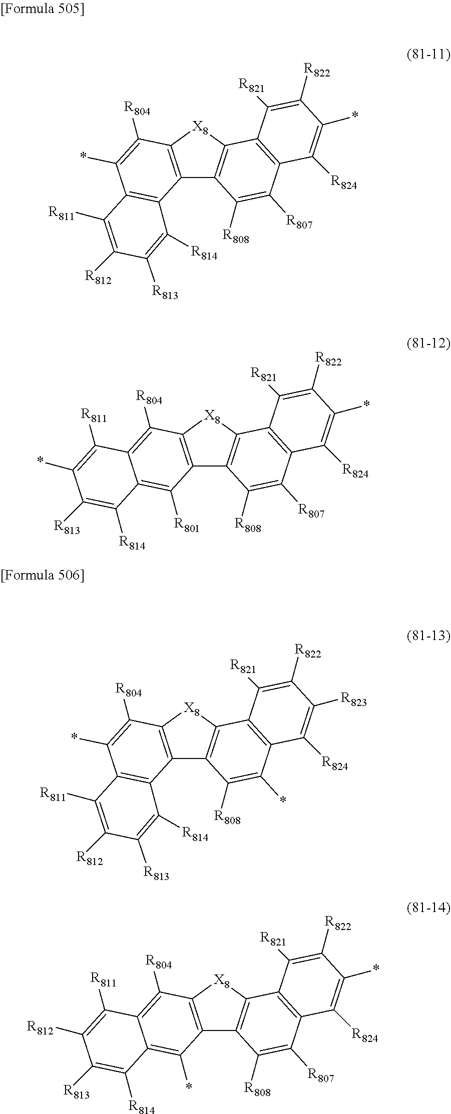

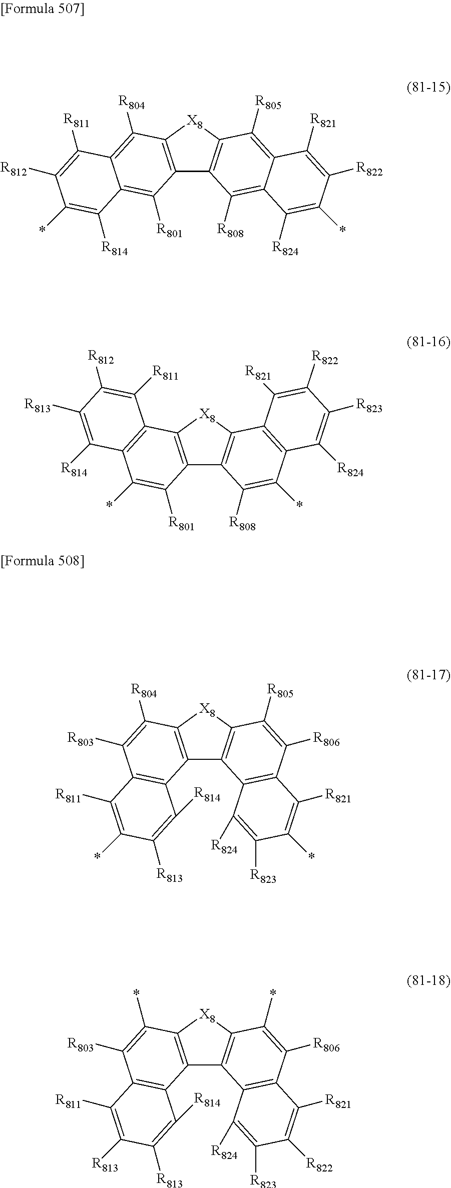

In the formulae (81-7) to (81-18):
X₈ represents the same as X₈ in the formula (8);
* is a single bond bonded to a monovalent group represented by the formula (84); and
R₈₀₁ to R₈₂₄ each independently represent the same as R₈₀₁ to R₈₂₄ in the formulae (81-1) to (81-6) not being the monovalent group represented by the formula (84).

R₈₀₁ to R₈₀₈ not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

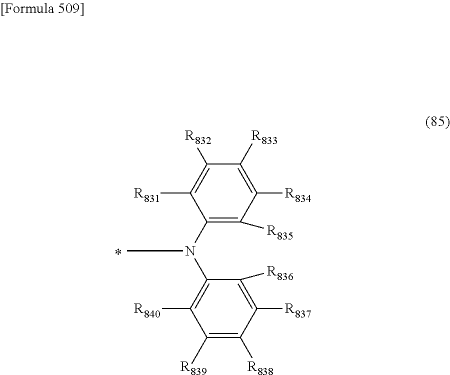

In the formula (85):
R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (85) represents the same as * in the formula (84).

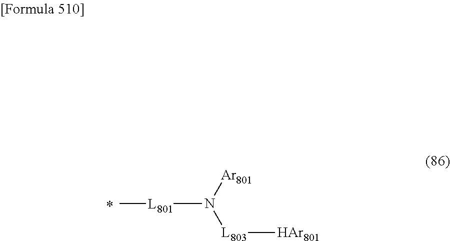

In the formula (86):
Ar₈₀₁, L₈₀₁, and L₈₀₃ represent the same as Ar₈₀₁, L₈₀₁, and L₈₀₃ in the formula (84); and

HAr₈₀₁ is a structure represented by a formula (87) below.

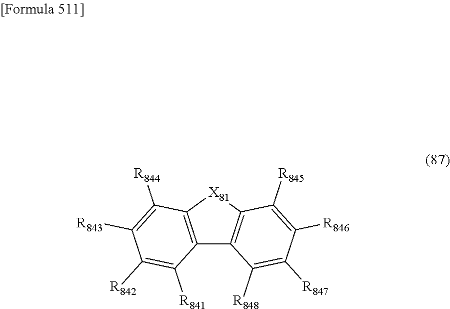

In the formula (87):
X₈₁ is an oxygen atom or a sulfur atom;
one of R₈₄₁ to R₈₄₈ is a single bond with L₈₀₃; and
R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (8)

Specific examples of the compound represented by the formula (8) include compounds given below as well as the compounds disclosed in WO 2014/104144.

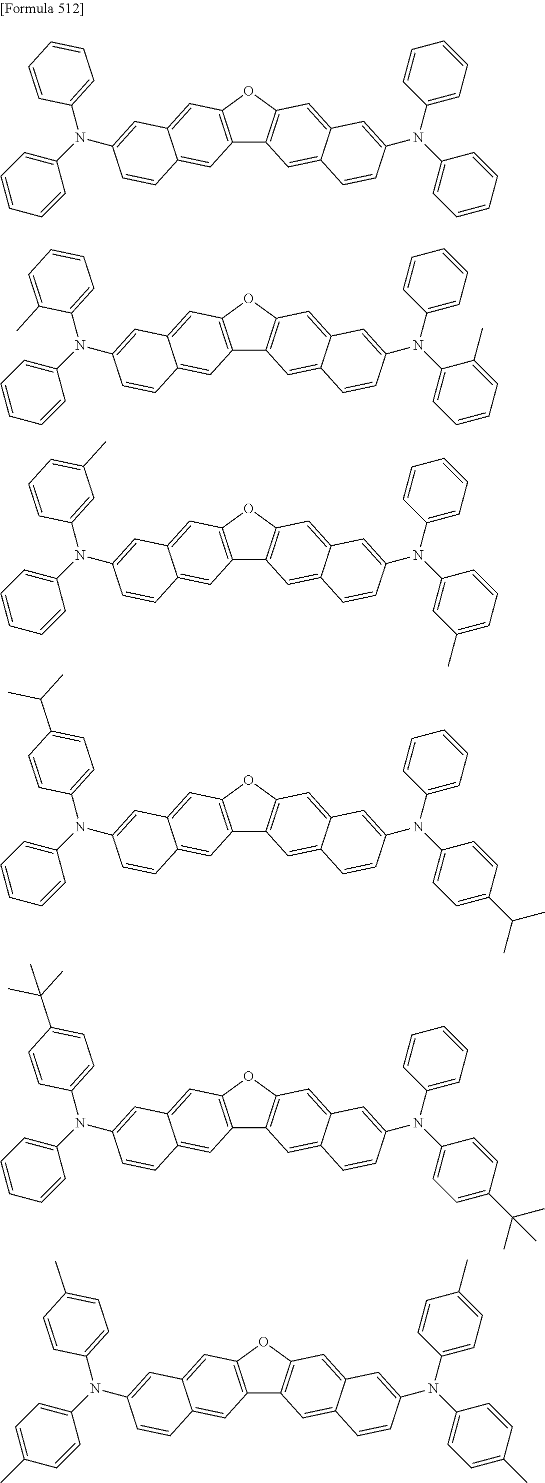

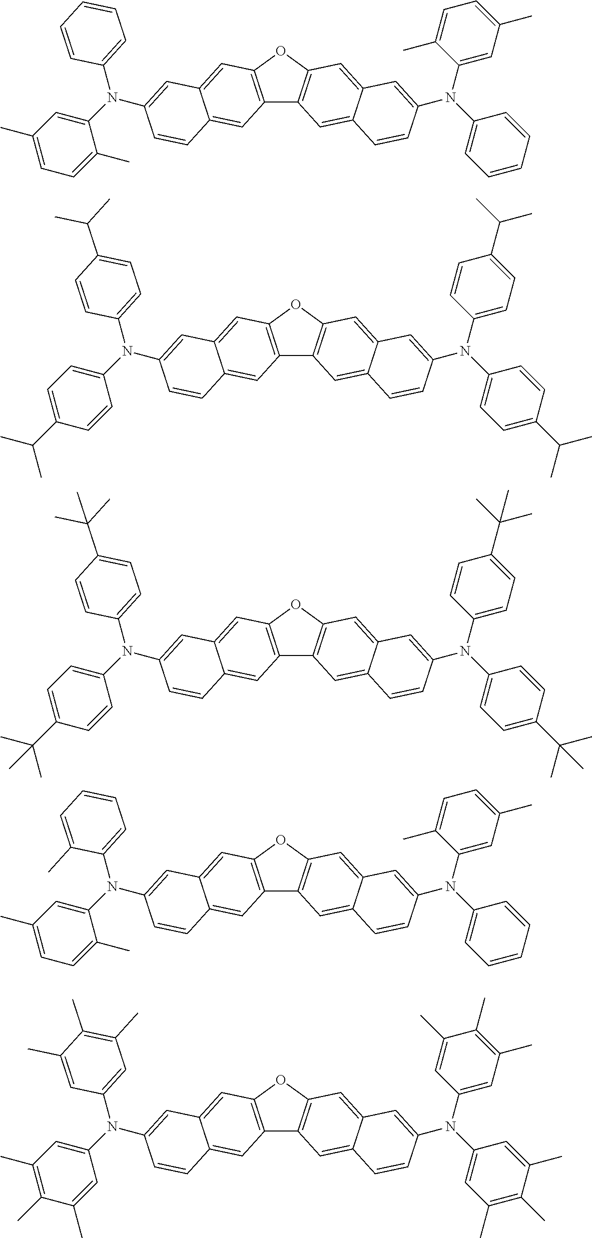

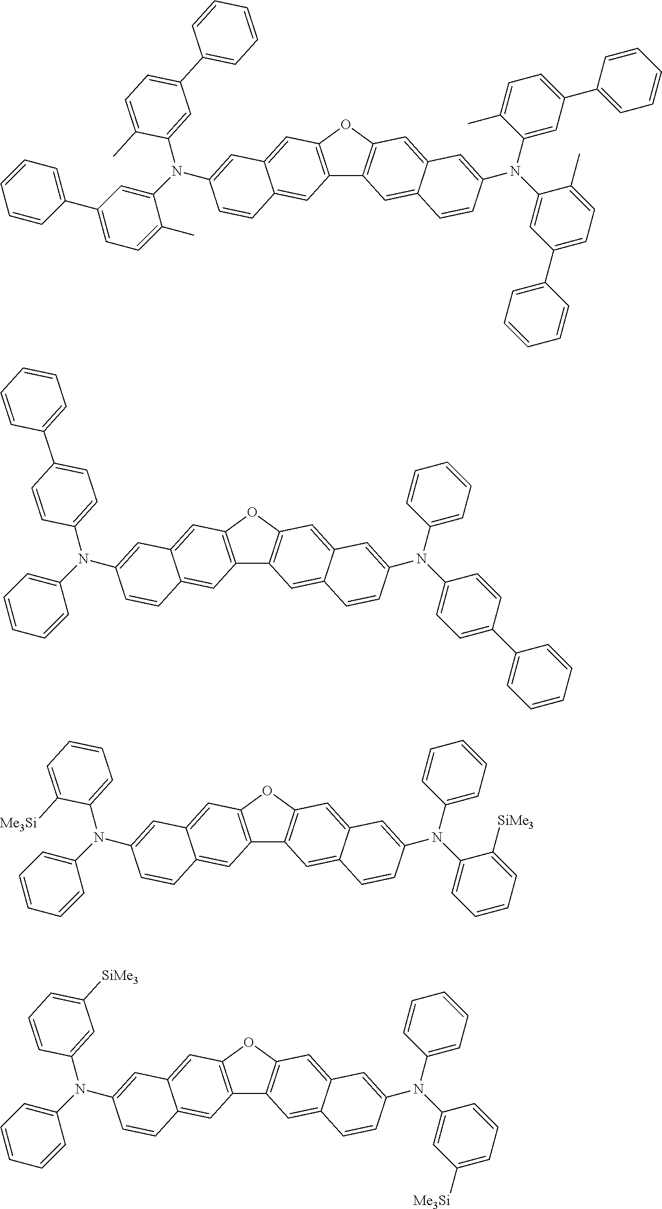 [SMILES: Cc1ccc(-c2ccccc2)cc1N(c1ccc2cc3c(cc2c1)oc1cc2cc(N(c4cc(-c5ccccc5)ccc4C)c4cc(-c5ccccc5)ccc4C)ccc2cc13)c1cc(-c2ccccc2)ccc1C ; c1ccc(-c2ccc(N(c3ccccc3)c3ccc4cc5c(cc4c3)oc3cc4cc(N(c6ccccc6)c6ccc(-c7ccccc7)cc6)ccc4cc35)cc2)cc1 ; C[Si](C)(C)c1ccccc1N(c1ccccc1)c1ccc2cc3c(cc2c1)oc1cc2cc(N(c4ccccc4)c4ccccc4[Si](C)(C)C)ccc2cc13 ; C[Si](C)(C)c1cccc(N(c2ccccc2)c2ccc3cc4c(cc3c2)oc2cc3cc(N(c5ccccc5)c5cccc([Si](C)(C)C)c5)ccc3cc24)c1]

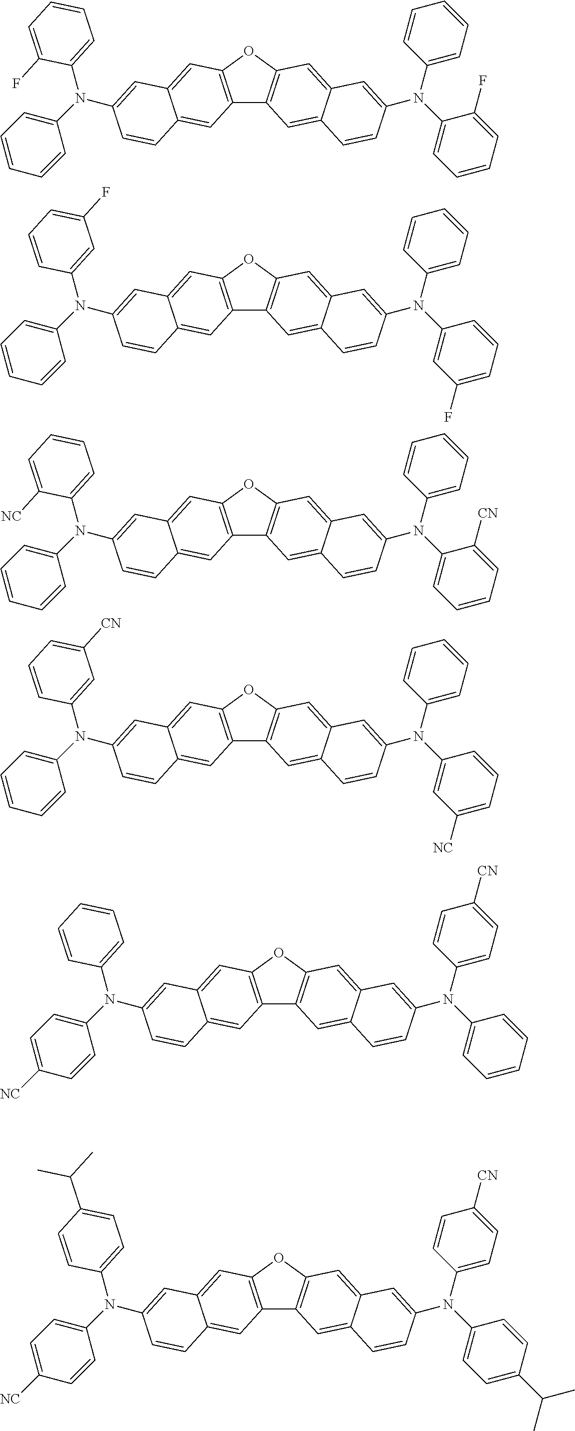

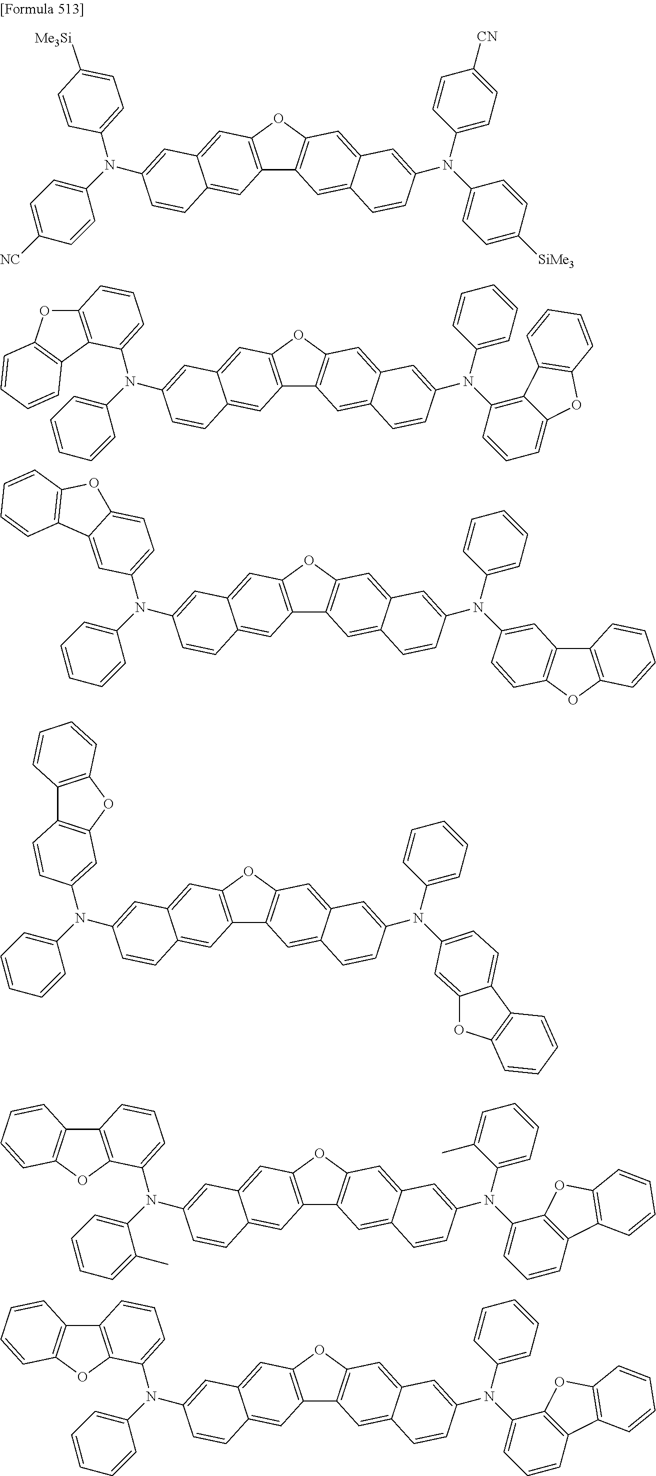

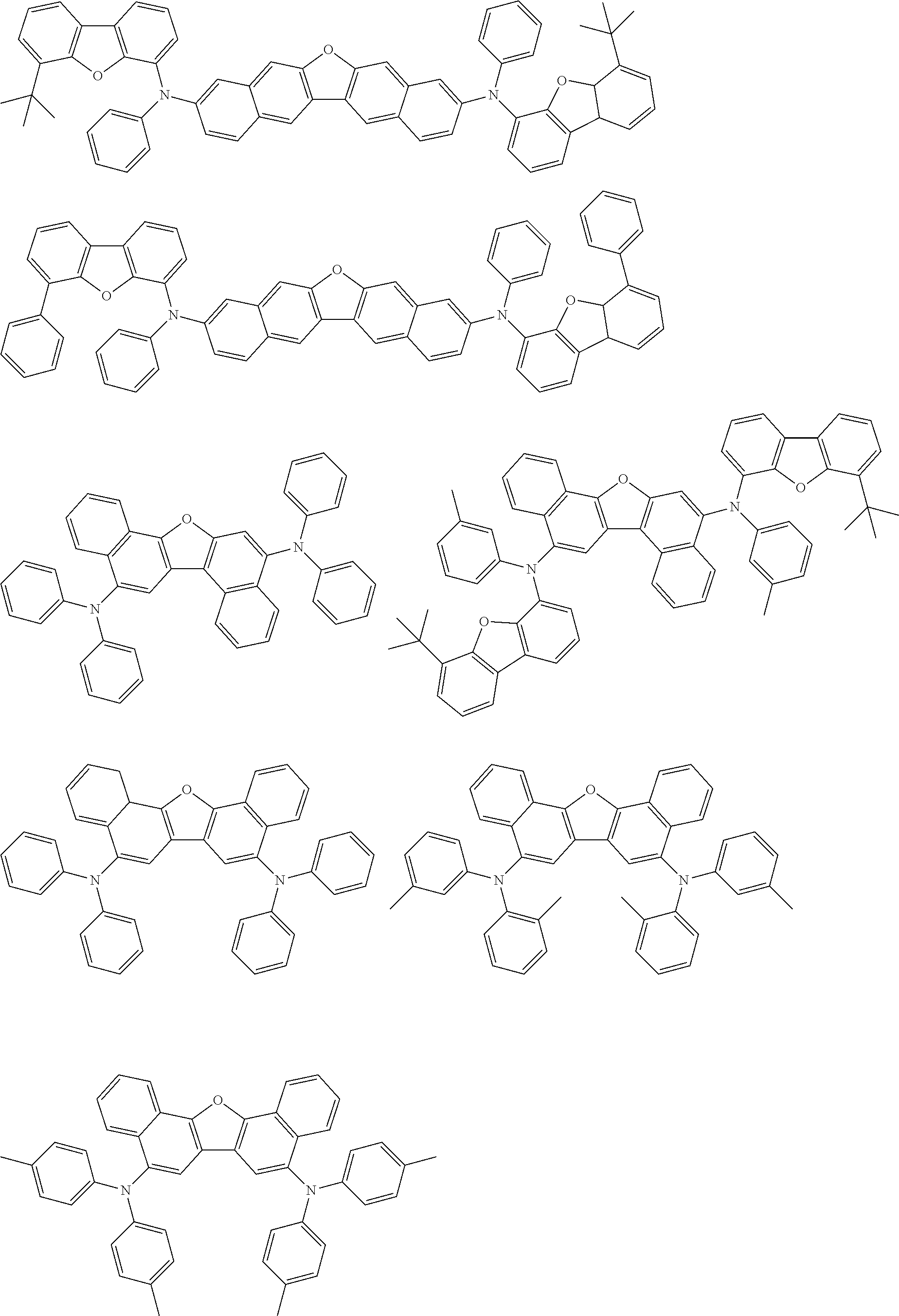

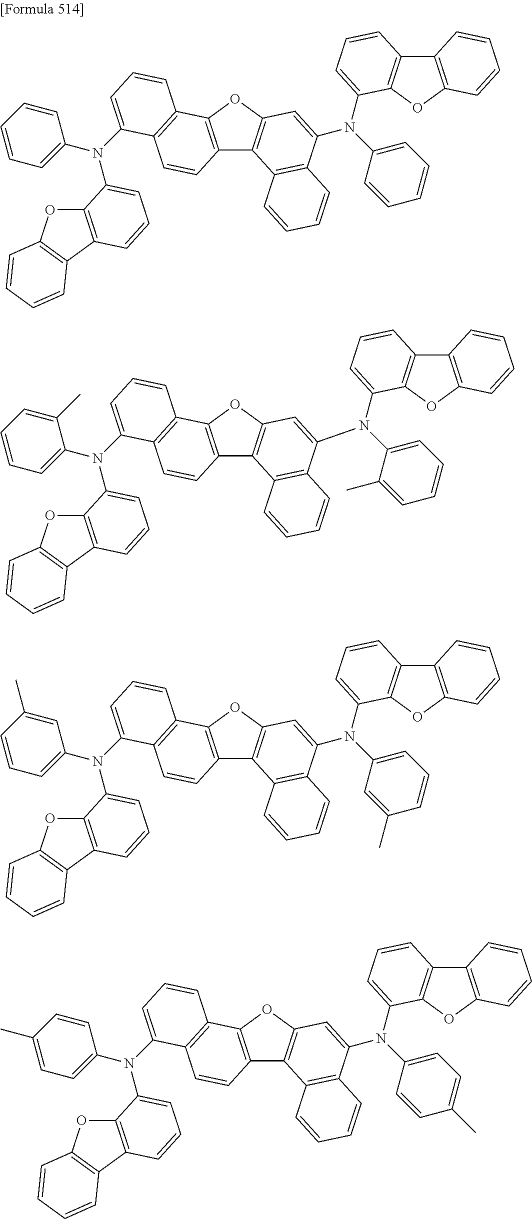 [SMILES: c1ccc(N(c2cccc3c2ccc2c3oc3cc(N(c4ccccc4)c4cccc5c4oc4ccccc45)c4ccccc4c32)c2cccc3c2oc2ccccc23)cc1 ; Cc1ccccc1N(c1cccc2c1ccc1c2oc2cc(N(c3ccccc3C)c3cccc4c3oc3ccccc34)c3ccccc3c21)c1cccc2c1oc1ccccc12 ; Cc1cccc(N(c2cccc3c2ccc2c3oc3cc(N(c4cccc(C)c4)c4cccc5c4oc4ccccc45)c4ccccc4c32)c2cccc3c2oc2ccccc23)c1 ; Cc1ccc(N(c2cccc3c2ccc2c3oc3cc(N(c4ccc(C)cc4)c4cccc5c4oc4ccccc45)c4ccccc4c32)c2cccc3c2oc2ccccc23)cc1]

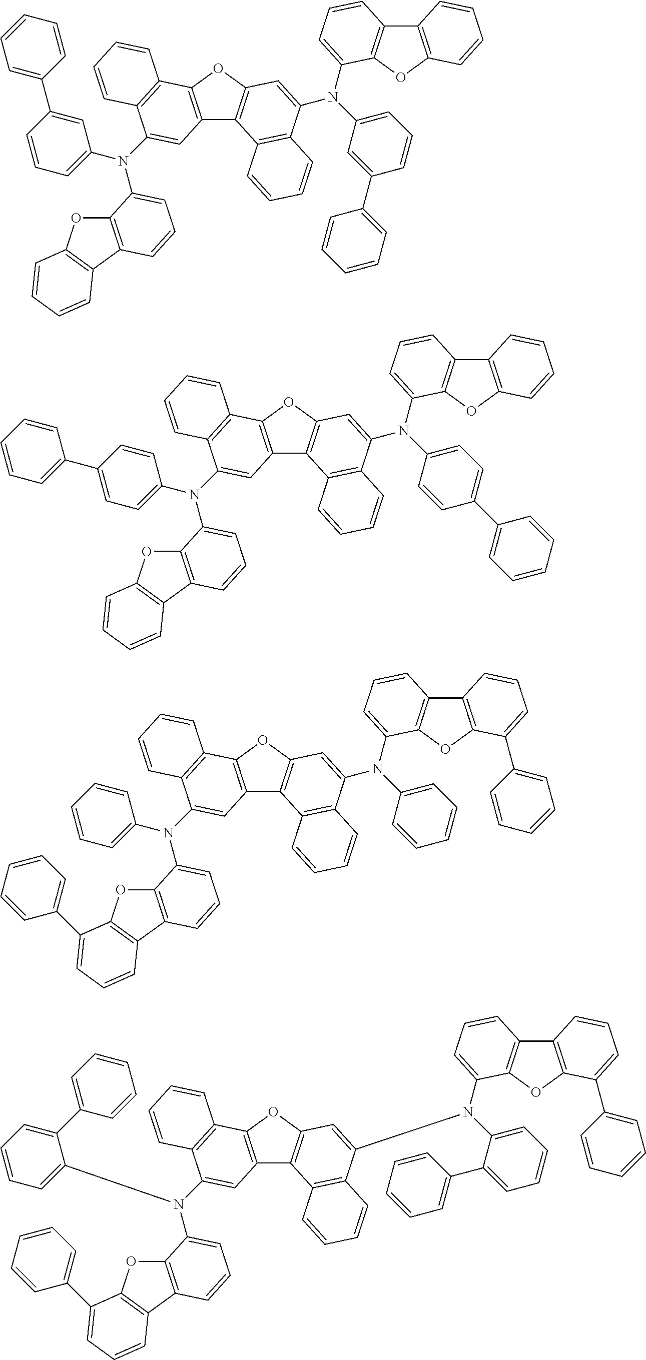

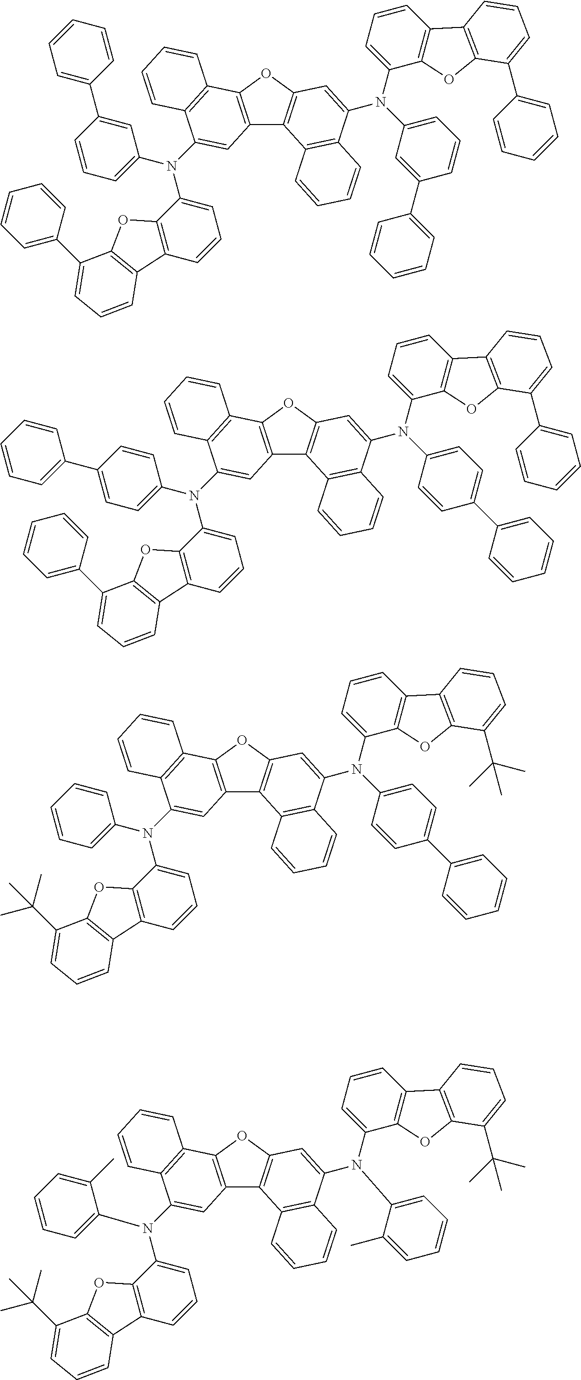

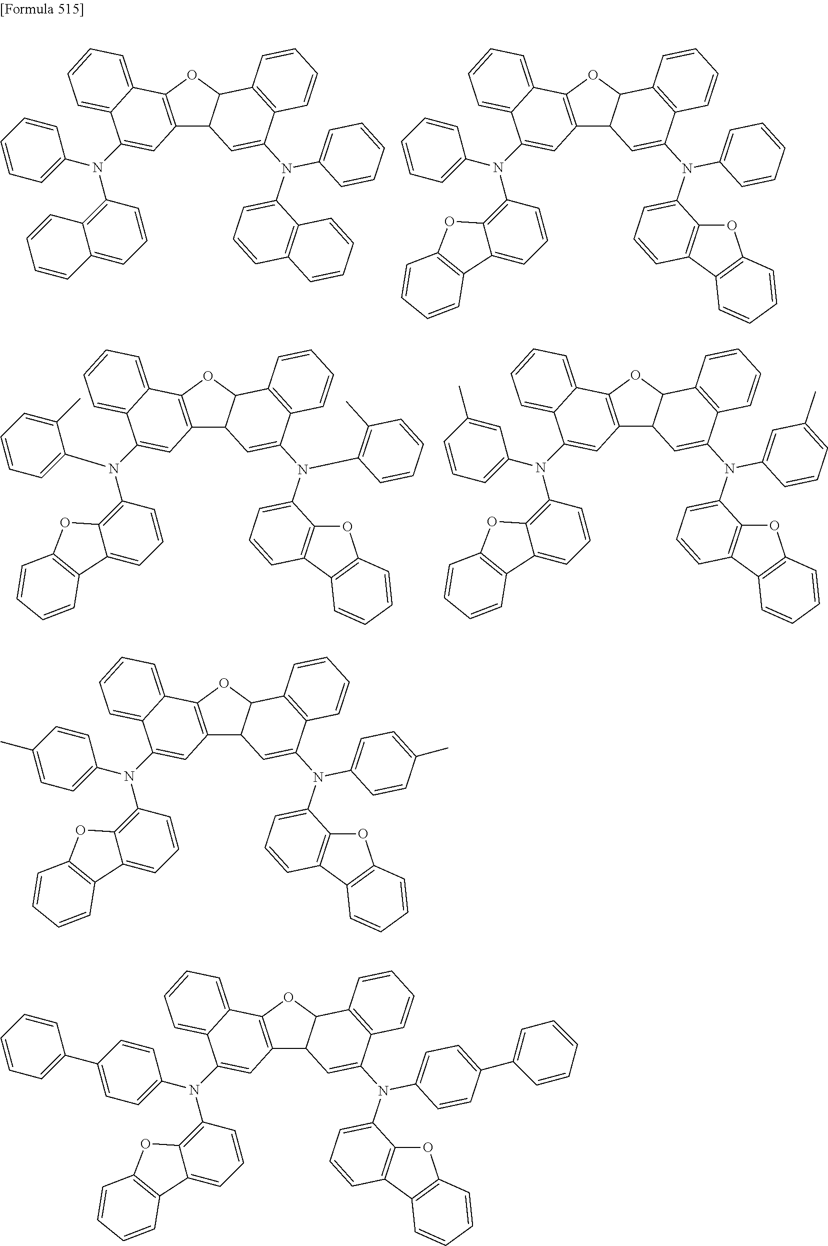

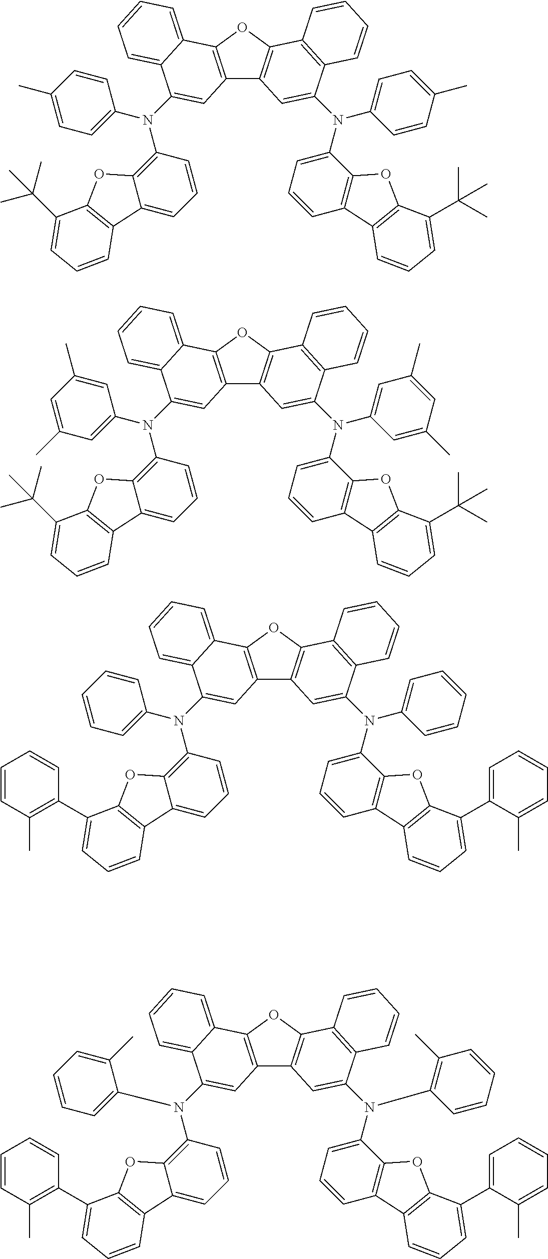

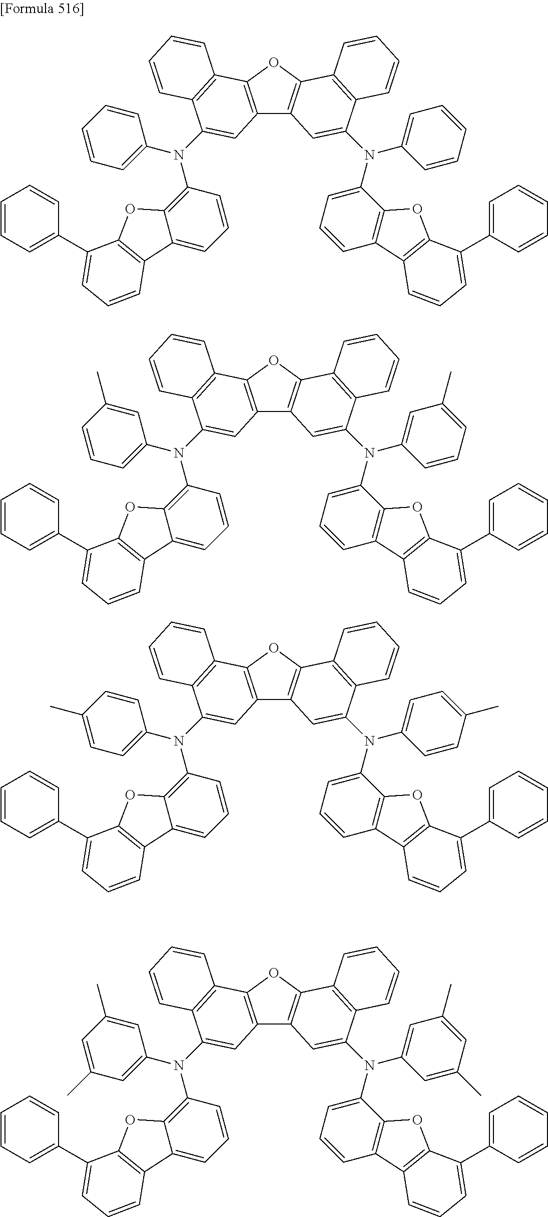

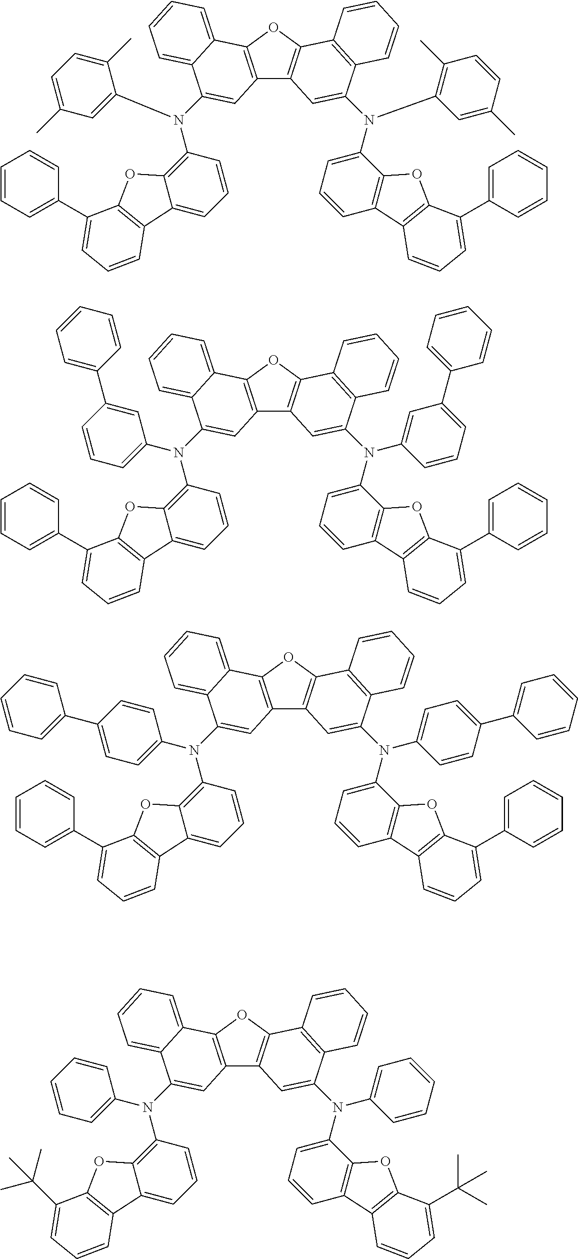

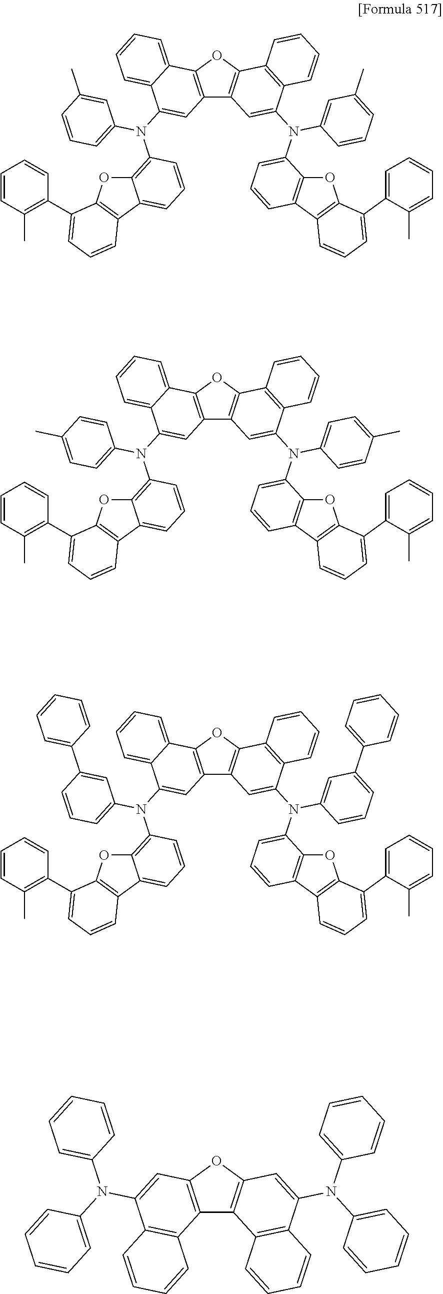 [SMILES: Cc1cccc(N(c2cc3c4cc(N(c5cccc(C)c5)c5cccc6c5oc5c(-c7ccccc7C)cccc56)c5ccccc5c4oc3c3ccccc23)c2cccc3c2oc2c(-c4ccccc4C)cccc23)c1 ; Cc1ccc(N(c2cc3c4cc(N(c5ccc(C)cc5)c5cccc6c5oc5c(-c7ccccc7C)cccc56)c5ccccc5c4oc3c3ccccc23)c2cccc3c2oc2c(-c4ccccc4C)cccc23)cc1 ; Cc1ccccc1-c1cccc2c1oc1c(N(c3cccc(-c4ccccc4)c3)c3cc4c5cc(N(c6cccc(-c7ccccc7)c6)c6cccc7c6oc6c(-c8ccccc8C)cccc67)c6ccccc6c5oc4c4ccccc34)cccc12 ; c1ccc(N(c2ccccc2)c2cc3oc4cc(N(c5ccccc5)c5ccccc5)c5ccccc5c4c3c3ccccc23)cc1]

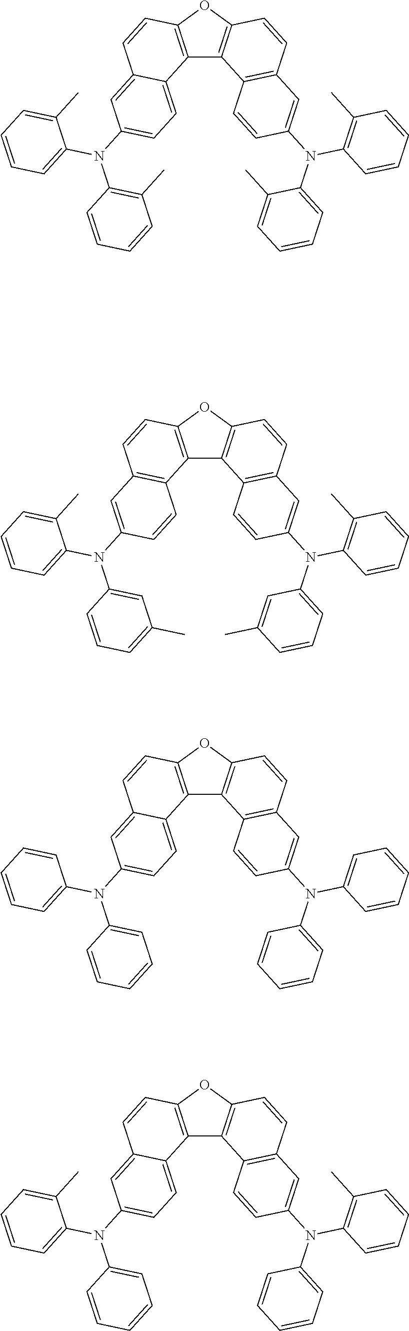 [SMILES: Cc1ccccc1N(c1ccc2c(ccc3oc4ccc5cc(N(c6ccccc6C)c6ccccc6C)ccc5c4c32)c1)c1ccccc1C ; Cc1cccc(N(c2ccc3c(ccc4oc5ccc6cc(N(c7cccc(C)c7)c7ccccc7C)ccc6c5c43)c2)c2ccccc2C)c1 ; c1ccc(N(c2ccccc2)c2ccc3c(ccc4oc5ccc6cc(N(c7ccccc7)c7ccccc7)ccc6c5c43)c2)cc1 ; Cc1ccccc1N(c1ccccc1)c1ccc2c(ccc3oc4ccc5cc(N(c6ccccc6)c6ccccc6C)ccc5c4c32)c1]

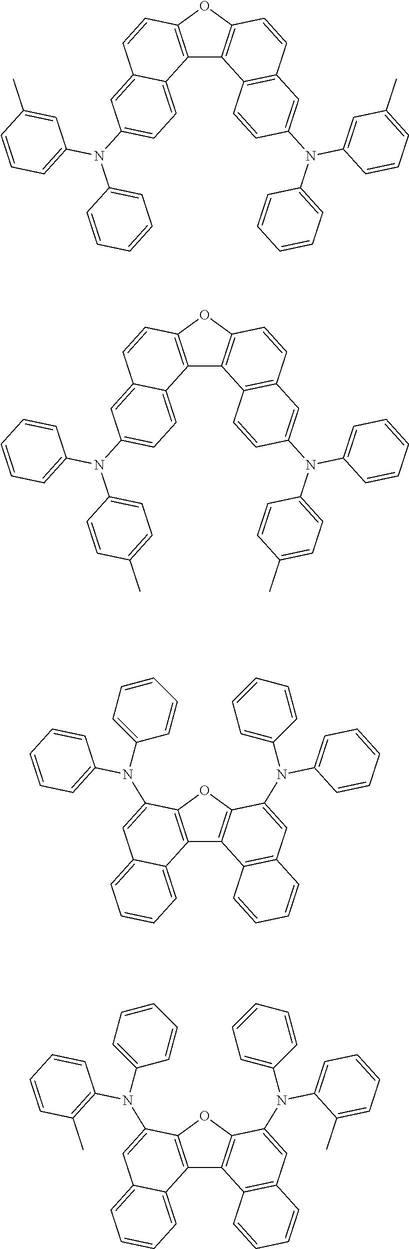 [SMILES: Cc1cccc(N(c2ccccc2)c2ccc3c(ccc4oc5ccc6cc(N(c7ccccc7)c7cccc(C)c7)ccc6c5c43)c2)c1 ; Cc1ccc(N(c2ccccc2)c2ccc3c(ccc4oc5ccc6cc(N(c7ccccc7)c7ccc(C)cc7)ccc6c5c43)c2)cc1 ; c1ccc(N(c2ccccc2)c2cc3ccccc3c3c2oc2c(N(c4ccccc4)c4ccccc4)cc4ccccc4c23)cc1 ; Cc1ccccc1N(c1ccccc1)c1cc2ccccc2c2c1oc1c(N(c3ccccc3)c3ccccc3C)cc3ccccc3c12]

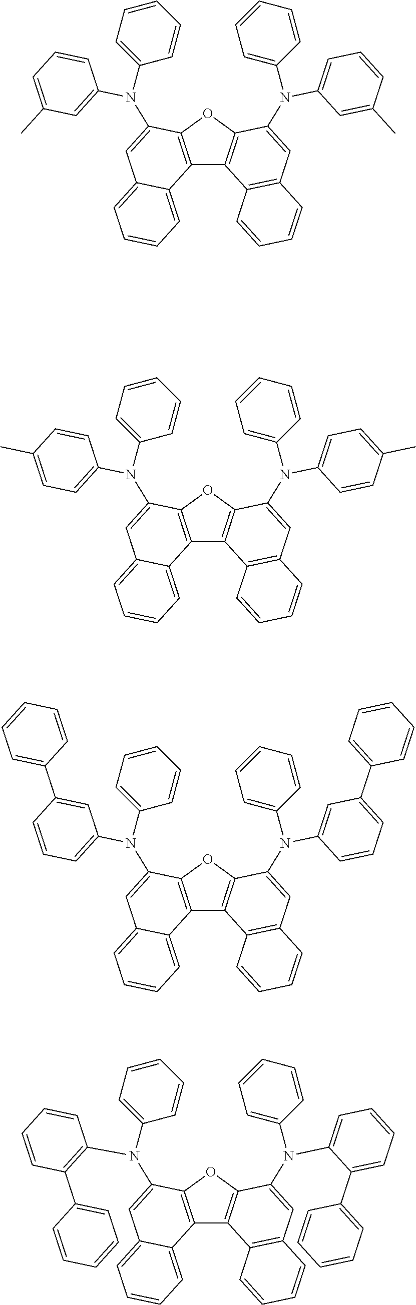

### Compound Represented by Formula (9)

The compound represented by the formula (9) will be described below.

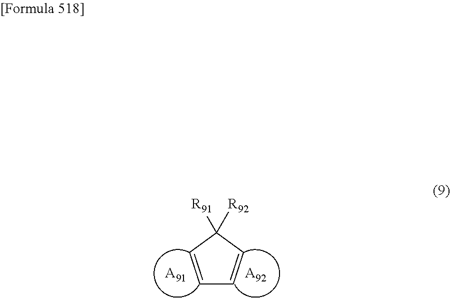

In the formula (9):
A₉₁ ring and A₉₂ ring are each independently a substituted or unsubstituted aromatic hydrocarbon ring having 6 to 50 ring carbon atoms, or a substituted or unsubstituted heterocycle having 5 to 50 ring atoms; and
at least one of the ring A₉₁ or the ring A₉₂ is bonded with * in a structure represented by a formula (92) below.

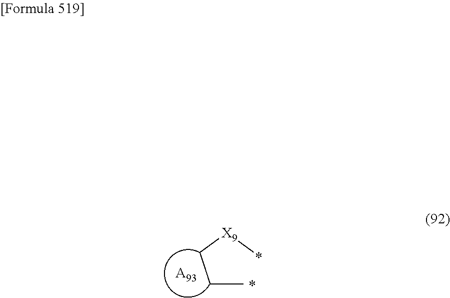

In the formula (92):
a ring A₉₃ is a substituted or unsubstituted aromatic hydrocarbon ring having 6 to 50 ring carbon atoms, or a substituted or unsubstituted heterocycle having 5 to 50 ring atoms;
X₉ is NR₉₃, C(R₉₄)(R₉₅), Si(R₉₆)(R₉₇), Ge(R₉₈)(R₉₉), an oxygen atom, a sulfur atom, or a selenium atom;
R₉₁ and R₉₂ are mutually bonded to form a substituted or unsubstituted monocyclic ring, mutually bonded to form a substituted or unsubstituted fused ring, or not mutually bonded;
R₉₁ and R₉₂ forming neither the substituted or unsubstituted monocyclic ring nor the substituted or unsubstituted fused ring, and and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of the ring A₉₁ or the ring A₉₂ is bonded to * in a structure represented by the formula (92). In other words, the ring carbon atoms of the aromatic hydrocarbon ring or the ring atoms of the heterocycle of the ring A₉₁ in an exemplary embodiment are bonded to * in a structure represented by the formula (92). Further, the ring carbon atoms of the aromatic hydrocarbon ring or the ring atoms of the heterocycle of the ring A₉₂ in an exemplary embodiment are bonded to * in a structure represented by the formula (92).

In an exemplary embodiment, a group represented by a formula (93) below is bonded to one or both of the ring A₉₁ and the ring A₉₂.

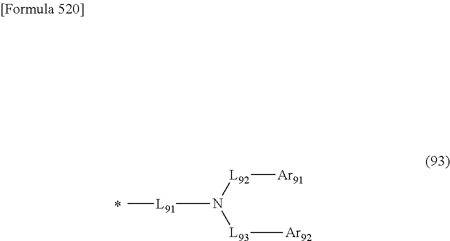

In the formula (93):
Ar₉₁ and Ar₉₂ are each independently a substituted or unsubstituted aryl group having 6 to 50 ring carbon atoms, or a substituted or unsubstituted heterocyclic group having 5 to 50 ring atoms; and
L₉₁ to L₉₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (93) represents a bonding position to one of the ring A₉₁ and the ring A₉₂.

In an exemplary embodiment, in addition to the ring A₉₁, the ring carbon atoms of the aromatic hydrocarbon ring or the ring atoms of the heterocycle of the ring A₉₂ are bonded to * in a structure represented by the formula (92). In this case, the structures represented by the formula (92) may be mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the rings A₉₁ and A₉₂ are each independently a substituted or unsubstituted aromatic hydrocarbon ring having 6 to 50 ring carbon atoms, example of which is a substituted or unsubstituted benzene ring.

In an exemplary embodiment, the ring A₉₃ is a substituted or unsubstituted aromatic hydrocarbon ring having 6 to 50 ring carbon atoms, example of which is a substituted or unsubstituted benzene ring.

In an exemplary embodiment, X₉ is an oxygen atom or a sulfur atom.

### Specific Examples of Compound Represented by Formula (9)

Specific examples of the compound represented by the formula (9) include compounds as below.

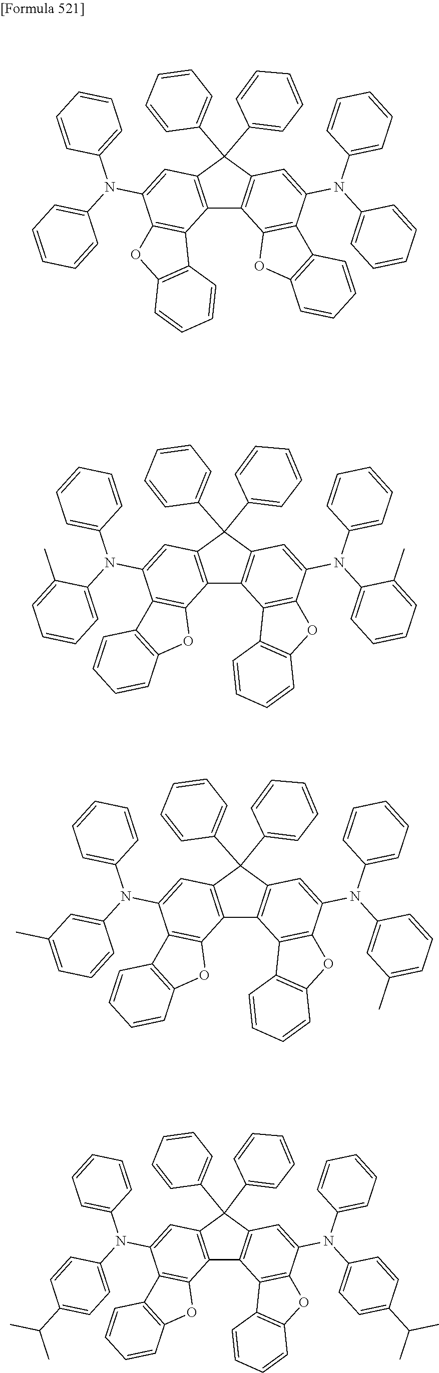 [SMILES: c1ccc(N(c2ccccc2)c2cc3c(c4c2oc2ccccc24)-c2c(cc(N(c4ccccc4)c4ccccc4)c4c2oc2ccccc24)C3(c2ccccc2)c2ccccc2)cc1 ; Cc1ccccc1N(c1ccccc1)c1cc2c(c3c1oc1ccccc13)-c1c(cc(N(c3ccccc3)c3ccccc3C)c3c1oc1ccccc13)C2(c1ccccc1)c1ccccc1 ; Cc1cccc(N(c2ccccc2)c2cc3c(c4c2oc2ccccc24)-c2c(cc(N(c4ccccc4)c4cccc(C)c4)c4c2oc2ccccc24)C3(c2ccccc2)c2ccccc2)c1 ; CC(C)c1ccc(N(c2ccccc2)c2cc3c(c4c2oc2ccccc24)-c2c(cc(N(c4ccccc4)c4ccc(C(C)C)cc4)c4c2oc2ccccc24)C3(c2ccccc2)c2ccccc2)cc1]

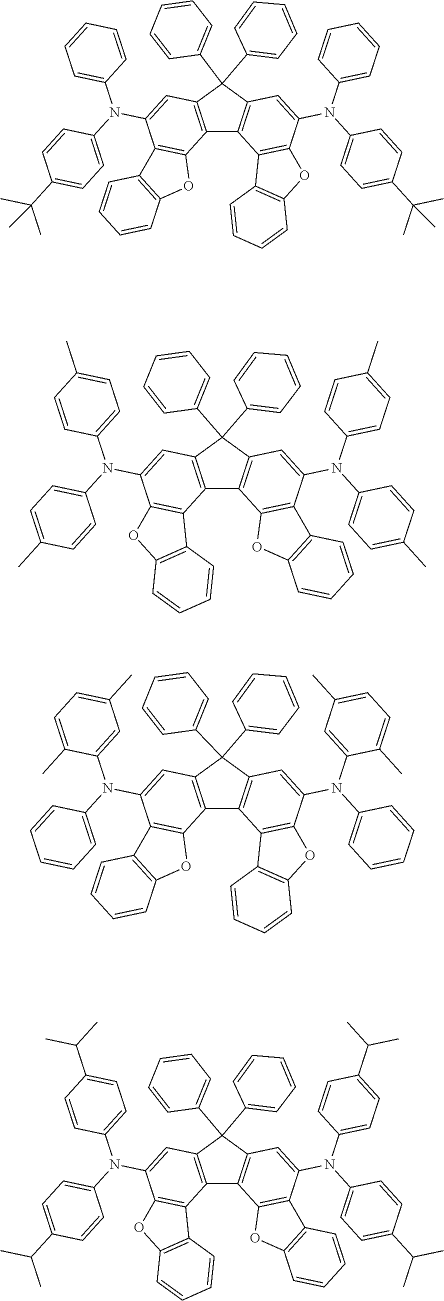

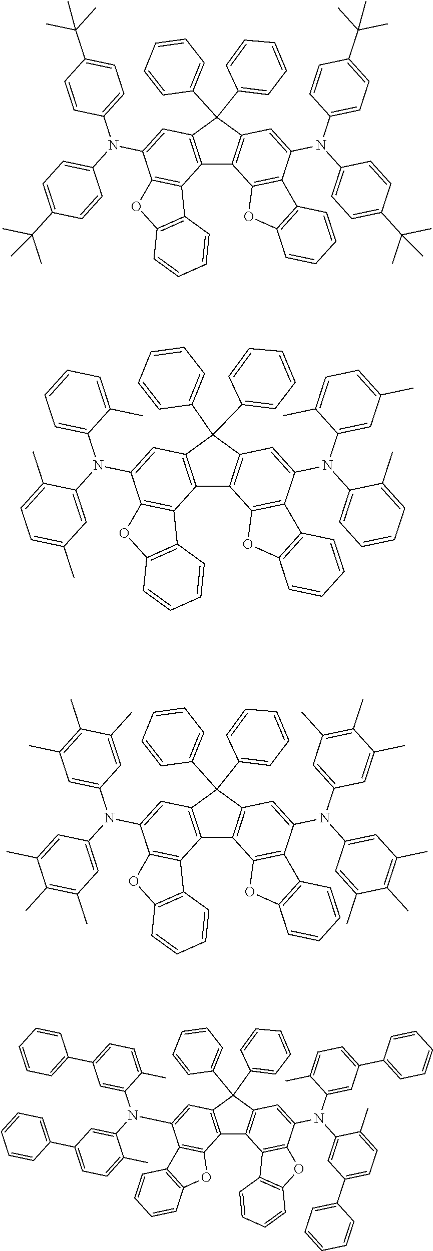

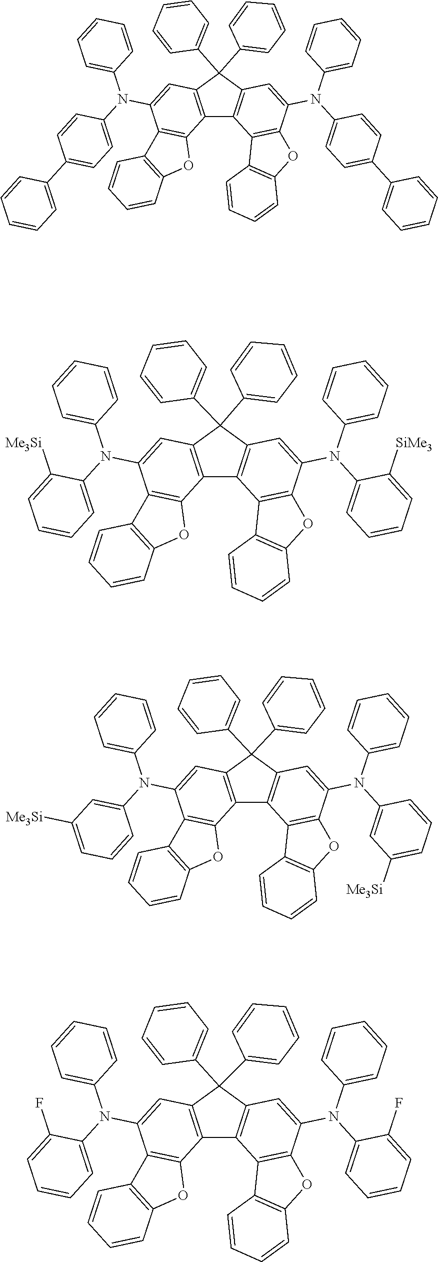 [SMILES: c1ccc(-c2ccc(N(c3ccccc3)c3cc4c(c5c3oc3ccccc35)-c3c(cc(N(c5ccccc5)c5ccc(-c6ccccc6)cc5)c5c3oc3ccccc35)C4(c3ccccc3)c3ccccc3)cc2)cc1 ; C[Si](C)(C)c1ccccc1N(c1ccccc1)c1cc2c(c3c1oc1ccccc13)-c1c(cc(N(c3ccccc3)c3ccccc3[Si](C)(C)C)c3c1oc1ccccc13)C2(c1ccccc1)c1ccccc1 ; C[Si](C)(C)c1cccc(N(c2ccccc2)c2cc3c(c4c2oc2ccccc24)-c2c(cc(N(c4ccccc4)c4cccc([Si](C)(C)C)c4)c4c2oc2ccccc24)C3(c2ccccc2)c2ccccc2)c1 ; Fc1ccccc1N(c1ccccc1)c1cc2c(c3c1oc1ccccc13)-c1c(cc(N(c3ccccc3)c3ccccc3F)c3c1oc1ccccc13)C2(c1ccccc1)c1ccccc1]

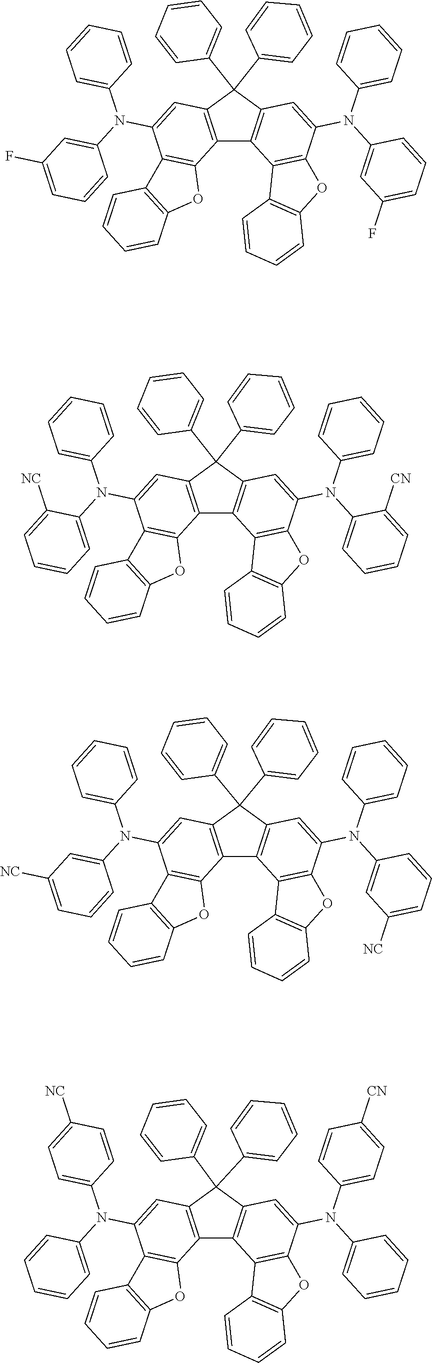 [SMILES: Fc1cccc(N(c2ccccc2)c2cc3c(c4c2oc2ccccc24)-c2c(cc(N(c4ccccc4)c4cccc(F)c4)c4c2oc2ccccc24)C3(c2ccccc2)c2ccccc2)c1 ; N#Cc1ccccc1N(c1ccccc1)c1cc2c(c3c1oc1ccccc13)-c1c(cc(N(c3ccccc3)c3ccccc3C#N)c3c1oc1ccccc13)C2(c1ccccc1)c1ccccc1 ; N#Cc1cccc(N(c2ccccc2)c2cc3c(c4c2oc2ccccc24)-c2c(cc(N(c4ccccc4)c4cccc(C#N)c4)c4c2oc2ccccc24)C3(c2ccccc2)c2ccccc2)c1 ; N#Cc1ccc(N(c2ccccc2)c2cc3c(c4c2oc2ccccc24)-c2c(cc(N(c4ccccc4)c4ccc(C#N)cc4)c4c2oc2ccccc24)C3(c2ccccc2)c2ccccc2)cc1]

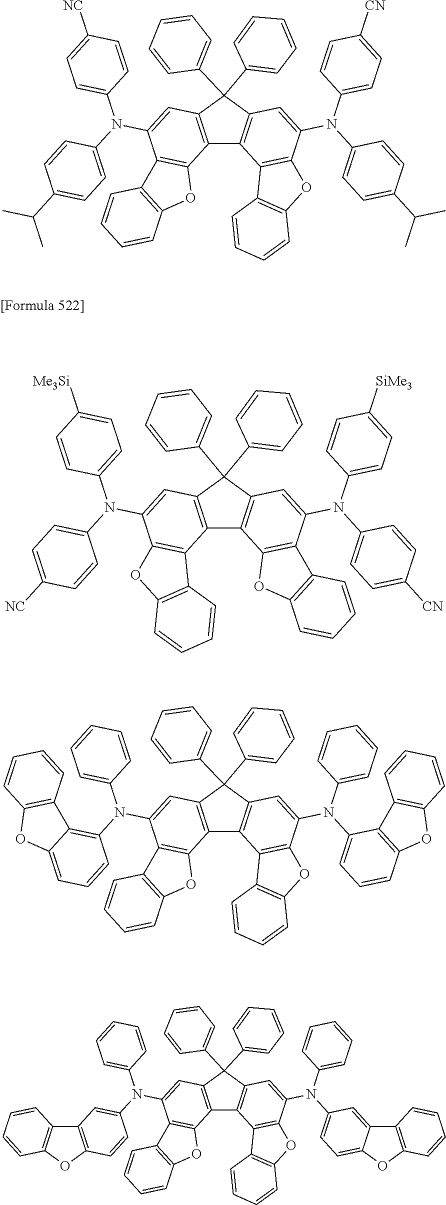

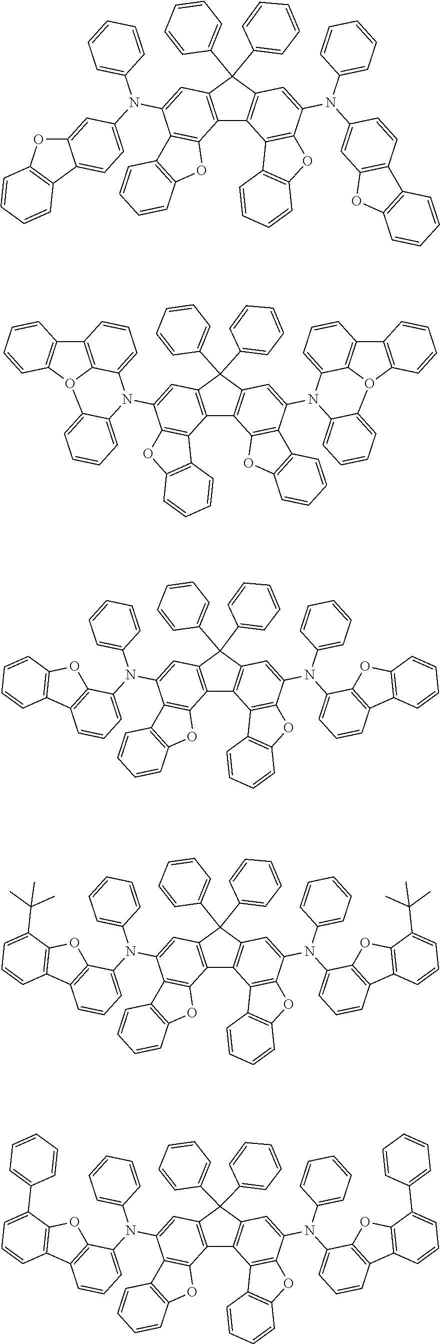

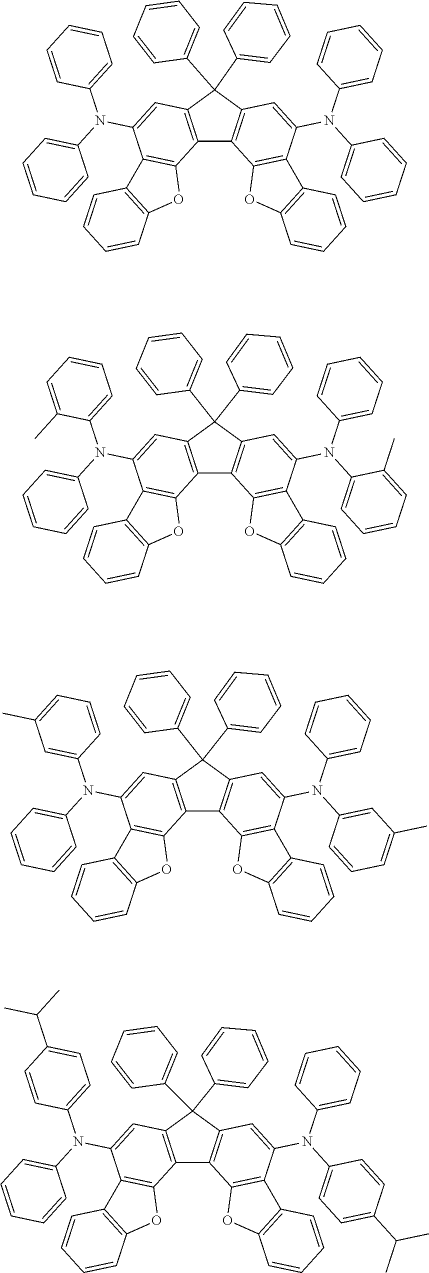

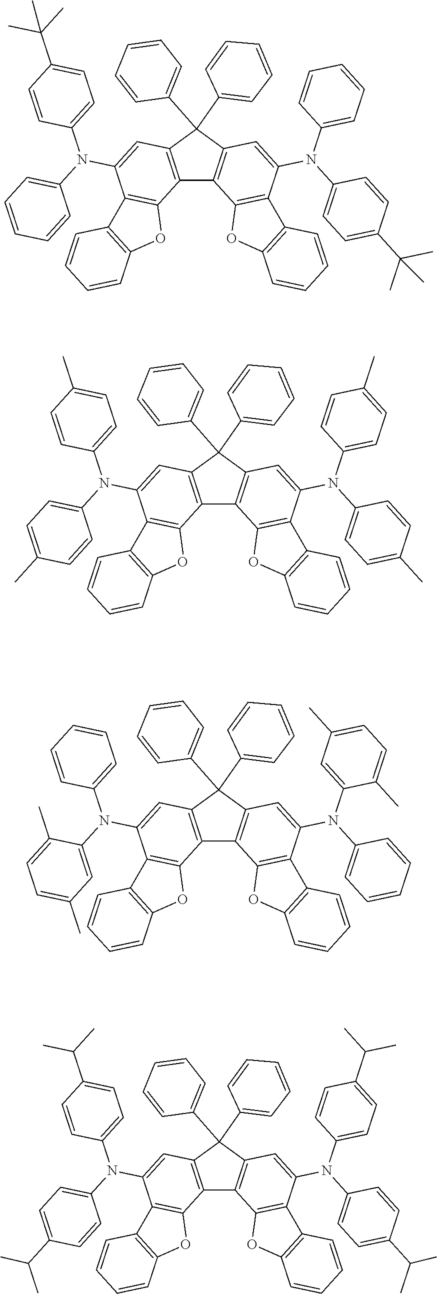

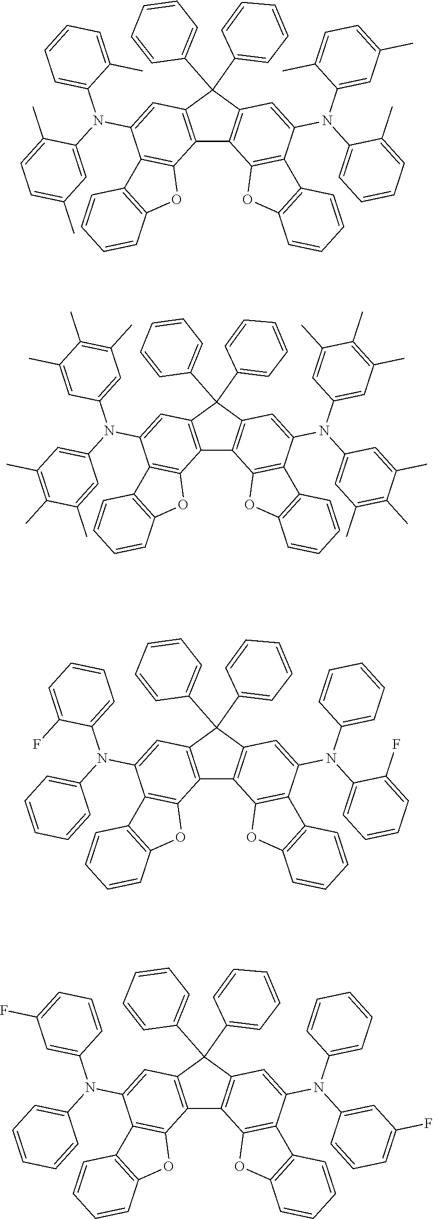

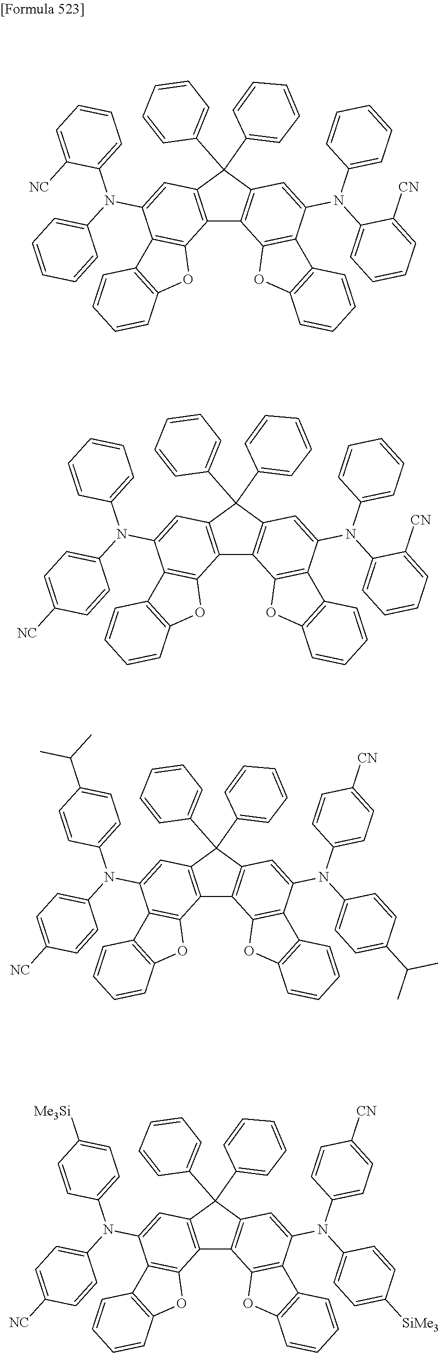 [SMILES: N#Cc1ccccc1N(c1ccccc1)c1cc2c(c3oc4ccccc4c13)-c1c(cc(N(c3ccccc3)c3ccccc3C#N)c3c1oc1ccccc13)C2(c1ccccc1)c1ccccc1 ; N#Cc1ccc(N(c2ccccc2)c2cc3c(c4oc5ccccc5c24)-c2c(cc(N(c4ccccc4)c4ccccc4C#N)c4c2oc2ccccc24)C3(c2ccccc2)c2ccccc2)cc1 ; CC(C)c1ccc(N(c2ccc(C#N)cc2)c2cc3c(c4oc5ccccc5c24)-c2c(cc(N(c4ccc(C#N)cc4)c4ccc(C(C)C)cc4)c4c2oc2ccccc24)C3(c2ccccc2)c2ccccc2)cc1 ; C[Si](C)(C)c1ccc(N(c2ccc(C#N)cc2)c2cc3c(c4oc5ccccc5c24)-c2c(cc(N(c4ccc(C#N)cc4)c4ccc([Si](C)(C)C)cc4)c4c2oc2ccccc24)C3(c2ccccc2)c2ccccc2)cc1]

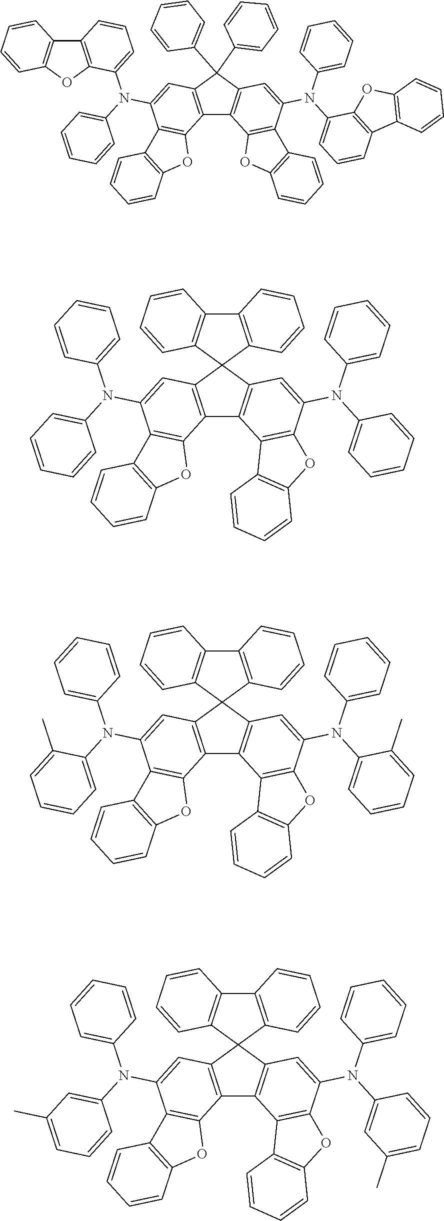 [SMILES: c1ccc(N(c2cccc3c2oc2ccccc23)c2cc3c(c4oc5ccccc5c24)-c2c(cc(N(c4ccccc4)c4cccc5c4oc4ccccc45)c4c2oc2ccccc24)C3(c2ccccc2)c2ccccc2)cc1 ; c1ccc(N(c2ccccc2)c2cc3c(c4c2oc2ccccc24)-c2c(cc(N(c4ccccc4)c4ccccc4)c4c2oc2ccccc24)C32c3ccccc3-c3ccccc32)cc1 ; Cc1ccccc1N(c1ccccc1)c1cc2c(c3c1oc1ccccc13)-c1c(cc(N(c3ccccc3)c3ccccc3C)c3c1oc1ccccc13)C21c2ccccc2-c2ccccc21 ; Cc1cccc(N(c2ccccc2)c2cc3c(c4c2oc2ccccc24)-c2c(cc(N(c4ccccc4)c4cccc(C)c4)c4c2oc2ccccc24)C32c3ccccc3-c3ccccc32)c1]

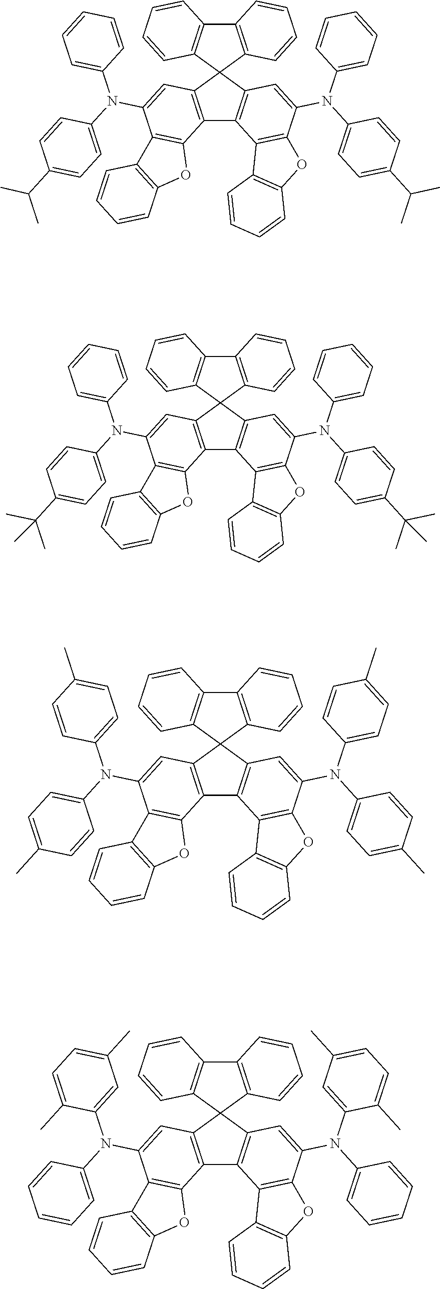

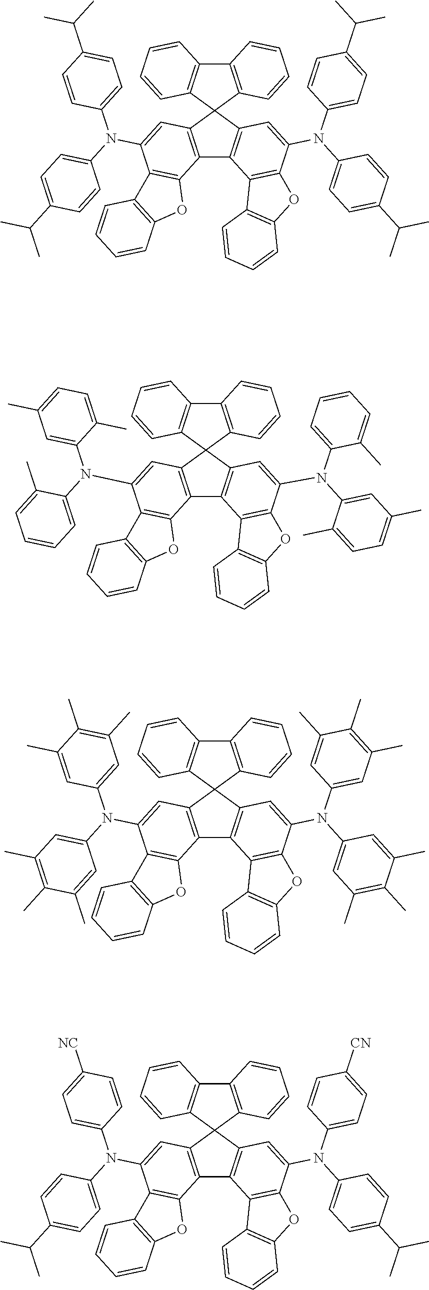

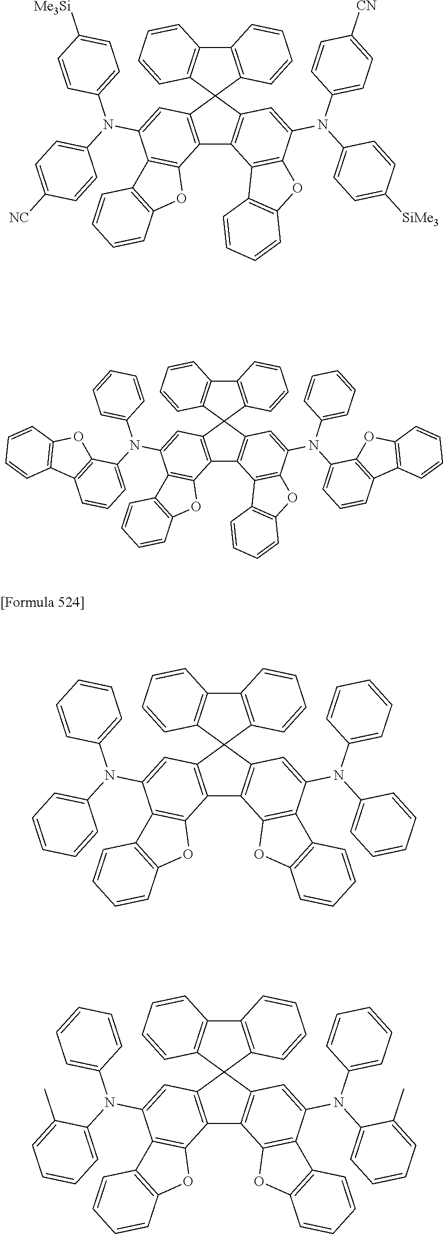 [SMILES: C[Si](C)(C)c1ccc(N(c2ccc(C#N)cc2)c2cc3c(c4c2oc2ccccc24)-c2c(cc(N(c4ccc(C#N)cc4)c4ccc([Si](C)(C)C)cc4)c4c2oc2ccccc24)C32c3ccccc3-c3ccccc32)cc1 ; c1ccc(N(c2cccc3c2oc2ccccc23)c2cc3c(c4c2oc2ccccc24)-c2c(cc(N(c4ccccc4)c4cccc5c4oc4ccccc45)c4c2oc2ccccc24)C32c3ccccc3-c3ccccc32)cc1 ; c1ccc(N(c2ccccc2)c2cc3c(c4oc5ccccc5c24)-c2c(cc(N(c4ccccc4)c4ccccc4)c4c2oc2ccccc24)C32c3ccccc3-c3ccccc32)cc1 ; Cc1ccccc1N(c1ccccc1)c1cc2c(c3oc4ccccc4c13)-c1c(cc(N(c3ccccc3)c3ccccc3C)c3c1oc1ccccc13)C21c2ccccc2-c2ccccc21]

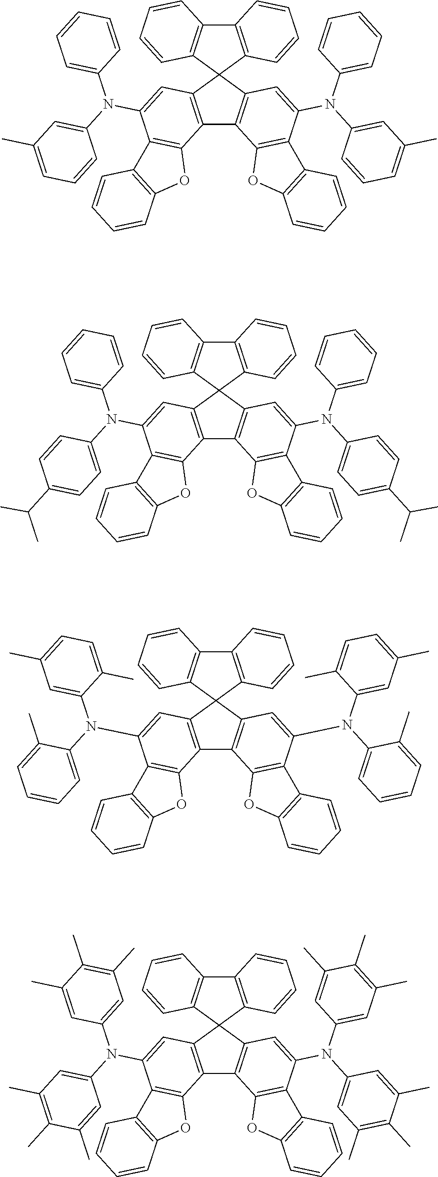

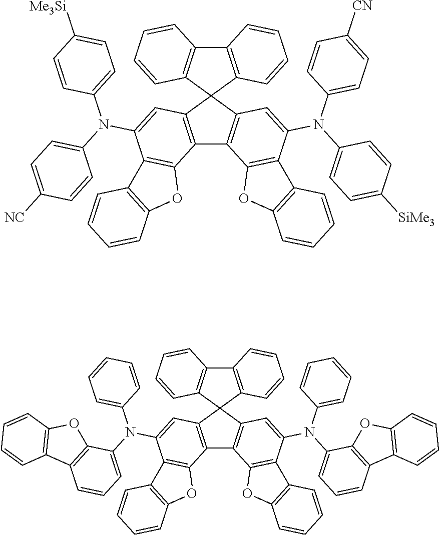 [SMILES: C[Si](C)(C)c1ccc(N(c2ccc(C#N)cc2)c2cc3c(c4oc5ccccc5c24)-c2c(cc(N(c4ccc(C#N)cc4)c4ccc([Si](C)(C)C)cc4)c4c2oc2ccccc24)C32c3ccccc3-c3ccccc32)cc1 ; c1ccc(N(c2cccc3c2oc2ccccc23)c2cc3c(c4oc5ccccc5c24)-c2c(cc(N(c4ccccc4)c4cccc5c4oc4ccccc45)c4c2oc2ccccc24)C32c3ccccc3-c3ccccc32)cc1]

### Compound Represented by Formula (10)

The compound represented by the formula (10) will be described below.

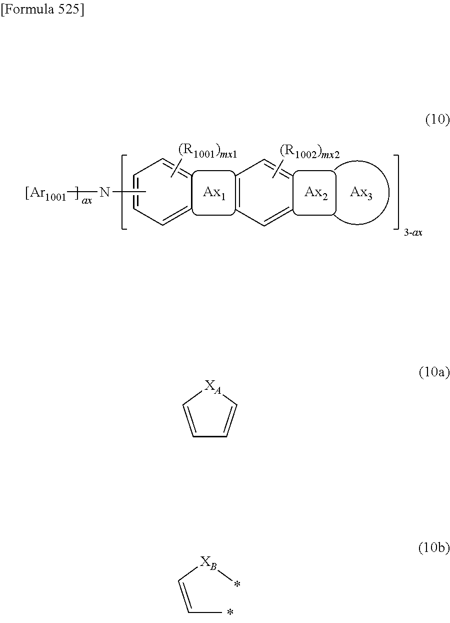

In the formula (10):
a ring Ax₁ is a ring represented by the formula (10a) that is fused with adjacent ring(s) at any position(s);
a ring Ax₂ is a ring represented by the formula (10b) that is fused with adjacent ring(s) at any position(s);
two * in the formula (10b) are bonded to a ring Ax₃ at any position(s);
X_{A} and X_{B} are each independently C(R₁₀₀₃)(R₁₀₀₄), Si(R₁₀₀₅)(R₁₀₀₆), an oxygen atom or a sulfur atom;
Ax₃ ring is a substituted or unsubstituted aromatic hydrocarbon ring having 6 to 50 ring carbon atoms, or a substituted or unsubstituted heterocycle having 5 to 50 ring atoms;
Ar₁₀₀₁ is a substituted or unsubstituted aryl group having 6 to 50 ring carbon atoms, or a substituted or unsubstituted heterocyclic group having 5 to 50 ring atoms;
R₁₀₀₁ to R₁₀₀₆ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
mx1 is 3, and mx2 is 2;
a plurality of R₁₀₀₁ are mutually the same or different;
ax is 0, 1, or 2;
when ax is 0 or 1, the structures enclosed by brackets indicated by “3-ax” are mutually the same or different; and
when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, Ax₃ ring is a substituted or unsubstituted aromatic hydrocarbon ring having 6 to 50 ring carbon atoms, example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

### Specific Examples of Compound Represented by Formula (10)

Specific examples of the compound represented by the formula (10) include compounds as below.

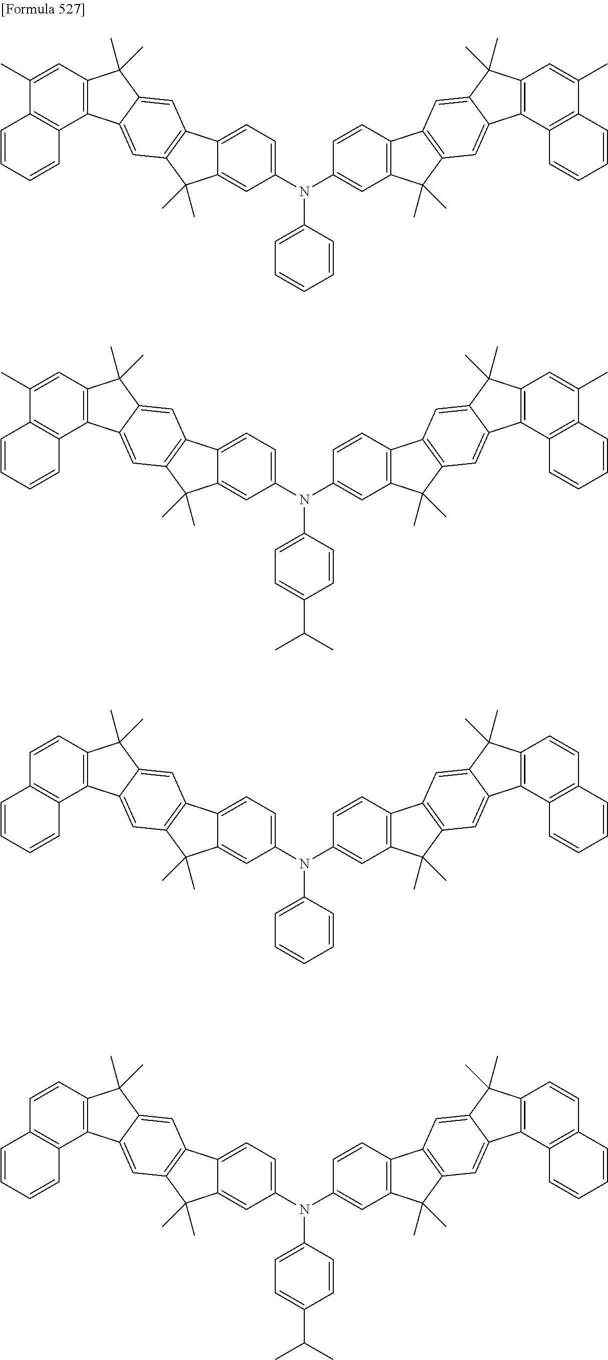

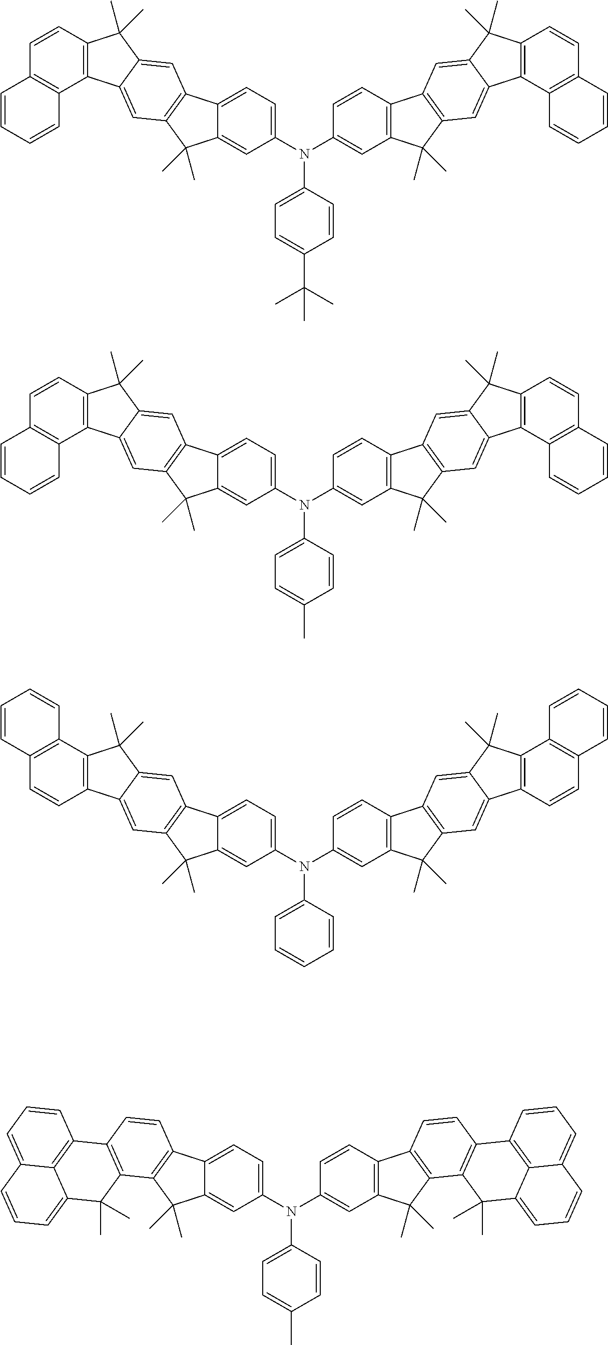 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; CC1(C)c2cc(N(c3ccccc3)c3ccc4c(c3)C(C)(C)c3cc5c(cc3-4)C(C)(C)c3c-5ccc4ccccc34)ccc2-c2cc3c(cc21)-c1ccc2ccccc2c1C3(C)C ; Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1]

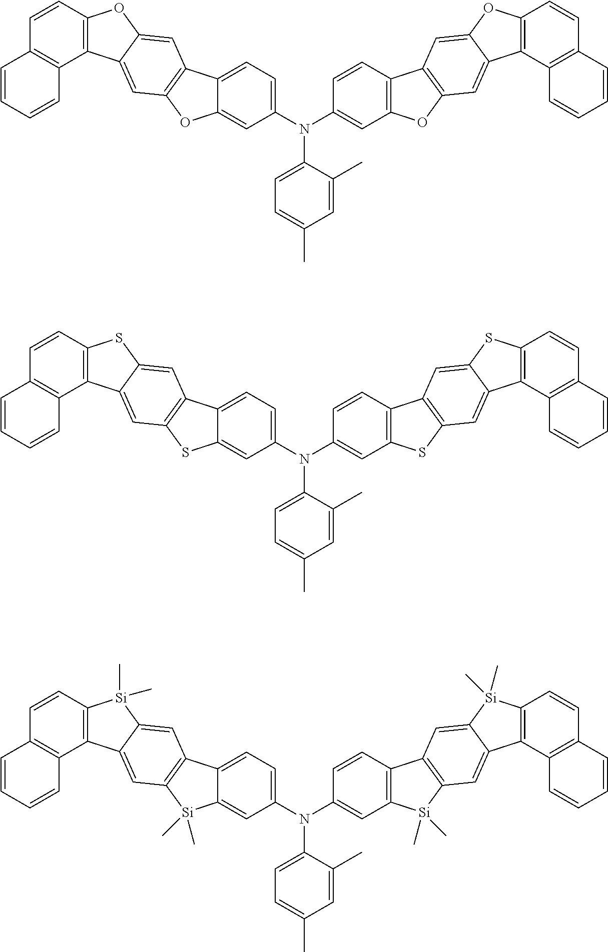 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the substituent for “the substituted or unsubstituted” group in each of the above formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, the substituent for “the substituted or unsubstituted” group in each of the above formulae is an unsubstituted alkyl group having 1 to 18 carbon atoms, an unsubstituted aryl group having 6 to 18 ring carbon atoms, or an unsubstituted heterocyclic group having 5 to 18 ring atoms.

### Electron Transporting Zone

An electron transporting zone is disposed between the emitting region and the cathode. An organic EL device of each exemplary embodiment may include a first electron transporting zone provided between the cathode and the first emitting region.

### Electron Transporting Layer

In an exemplary arrangement of the organic EL device of each exemplary embodiment, the first electron transporting zone includes at least one electron transporting layer. The at least one electron transporting layer in the first electron transporting zone contains a nitrogen-containing compound that has at least one of a five-membered ring having a nitrogen atom or a six-membered ring having a nitrogen atom.

In an exemplary arrangement of the organic EL device of each exemplary embodiment, the at least one electron transporting layer in the electron transporting zone contains, as a nitrogen-containing compound, at least one compound selected from the group consisting of an imidazole derivative, benzimidazole derivative, azine derivative, carbazole derivative, and phenanthroline derivative.

In an exemplary arrangement of the organic EL device of each exemplary embodiment, the at least one electron transporting layer in the first electron transporting zone contains, as a nitrogen-containing compound, at least one compound selected from the group consisting of an imidazole derivative, benzimidazole derivative, azine derivative, carbazole derivative, and phenanthroline derivative.

### Phenanthroline Derivative

In an exemplary arrangement of the organic EL device of each exemplary embodiment, at least one electron transporting layer in the electron transporting zone contains a phenanthroline derivative as a nitrogen-containing compound.

In an exemplary arrangement of the organic EL device of each exemplary embodiment, the phenanthroline derivative (phenanthroline compound) contained in the electron transporting layer is a compound represented by a formula (20) below and having at least one group represented by a formula (21) below.

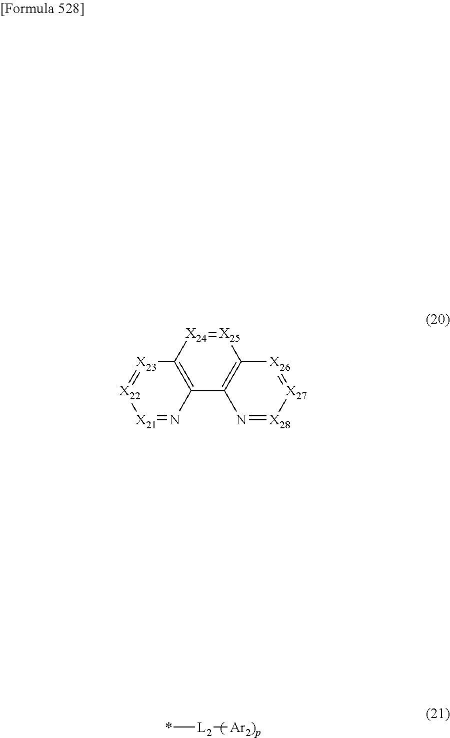

In the formula (20):
X₂₁ to X₂₈ are each independently a nitrogen atom, CR₂₁, or a carbon atom bonded to a group represented by the formula (21);
at least one of X₂₁ to X₂₈ is a carbon atom bonded to a group represented by the formula (21);
when a plurality of groups represented by the formula (21) are present, the plurality of groups represented by the formula (21) are mutually the same or different;
at least one combination of adjacent two or more of a plurality of R₂₁ are mutually bonded to form a substituted or unsubstituted monocyclic ring, mutually bonded to form a substituted or unsubstituted fused ring, or not mutually bonded; and
R₂₁ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a group represented by —C(═O) R₉₃₁, a group represented by —COOR₉₃₂, a group represented by —S(═O)₂R₉₃₃, a group represented by —B(R₉₃₄)(R₉₃₅), a group represented by —P(═O)(R₉₃₆)(R₉₃₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (21):
Ar₂ is a substituted or unsubstituted aryl group having 6 to 50 ring carbon atoms, or a substituted or unsubstituted heterocyclic group having 5 to 50 ring atoms;
p is 1, 2, 3, 4, or 5;
when two or more Ar₂ are present, the two or more Ar₂ are mutually the same or different;
L₂ is a single bond or a linking group;
L₂ as the linking group is a substituted or unsubstituted polyvalent linear, branched or cyclic aliphatic hydrocarbon group having 1 to 50 carbon atoms, a substituted or unsubstituted polyvalent aromatic hydrocarbon group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₂ as the multiple linking group are mutually the same or different, and adjacent ones thereof are mutually bonded to form a ring, or not mutually bonded;
Ar₂ and L₂ as the linking group are mutually bonded to form a ring, or not mutually bonded;
L₂ as the linking group, and a carbon atom in one of X₂₁ to X₂₈ adjacent to a carbon atom bonded to L₂, or R₂₁ in CR₂₁ are mutually bonded to form a ring, or not mutually bonded; and
* in the formula (21) represents a bonding position to a ring represented by the formula (20);
In the phenanthroline compound, R₉₀₁, R₉₀₂, R₉₀₃, R₉₀₄, R₉₀₅, R₉₀₆, R₉₀₇, R₉₃₁, R₉₃₂, R₉₃₃, R₉₃₄, R₉₃₅, R₉₃₆ and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₃₁ are present, the plurality of R₉₃₁ are mutually the same or different;
when a plurality of R₉₃₂ are present, the plurality of R₉₃₂ are mutually the same or different;
when a plurality of R₉₃₃ are present, the plurality of R₉₃₃ are mutually the same or different;
when a plurality of R₉₃₄ are present, the plurality of R₉₃₄ are mutually the same or different;
when a plurality of R₉₃₅ are present, the plurality of R₉₃₅ are mutually the same or different;
when a plurality of R₉₃₆ are present, the plurality of R₉₃₆ are mutually the same or different; and
when a plurality of R₉₃₇ are present, the plurality of R₉₃₇ are mutually the same or different.

A group represented by —O—(R₉₀₄) herein in which R₉₀₄ is a hydrogen atom is a hydroxy group.

A group represented by —S—(R₉₀₅) herein in which R₉₀₅ is a hydrogen atom is a thiol group.

A group represented by —S(═O)₂R₉₃₃ herein in which R₉₃₃ is a substituent is a substituted sulfo group.

A group represented by —B(R₉₃₄)(R₉₃₅) herein in which R₉₃₄ and R₉₃₅ are each a substituent is a substituted boryl group.

A group represented by —P(═O)(R₉₃₆)(R₉₃₇) herein in which R₉₃₆ and R₉₃₇ are each a substituent is a substituted phosphine oxide group, and a group represented by —P(═O)(R₉₃₆)(R₉₃₇) herein in which R₉₃₆ and R₉₃₇ are each an aryl group is an aryl phosphoryl group.

An “unsubstituted polyvalent linear, branched or cyclic aliphatic hydrocarbon group” mentioned herein has, unless otherwise specified herein, 1 to 50, preferably 1 to 20, more preferably 1 to 6 carbon atoms.

An “unsubstituted polyvalent aromatic hydrocarbon group” mentioned herein has, unless otherwise specified herein, 6 to 50, preferably 6 to 30, more preferably 6 to 18 ring carbon atoms.

An “unsubstituted polyvalent heterocyclic group” mentioned herein has, unless otherwise specified herein, 5 to 50, preferably 5 to 30, more preferably 5 to 18 ring atoms.

In an exemplary embodiment, a heterocyclic group having 5 to 50 ring atoms in Ar₂ of the formula (21) contains a substituted or unsubstituted group derived from a cyclic structure represented by the formula (20).

In an exemplary embodiment, X₂₁ and X₂₈ in the formula (20) are each a carbon atom bonded to a group represented by the formula (21).

In an exemplary embodiment, one of X₂₁ and X₂₈ in the formula (20) is a carbon atom bonded to a group represented by the formula (21), and the other of X₂₁ and X₂₈ in the formula (20) is a carbon atom bonded to a hydrogen atom.

In an exemplary embodiment, X₂₁ to X₂₈ in the formula (20) are each independently CR₂₁ or a carbon atom bonded to a group represented by the formula (21).

In an exemplary embodiment, X₂₁ to X₂₈ in the formula (20) not being the carbon atom bonded to the group represented by the formula (21) are each CR₂₁. In other words, in an exemplary embodiment, the compound represented by the formula (20) is a 1,10-phenanthroline derivative.

In an exemplary embodiment, Ar₂ in the formula (21) is a substituted or unsubstituted fused aromatic hydrocarbon group having 8 to 20 ring carbon atoms.

In an exemplary embodiment, the fused aromatic hydrocarbon group having 8 to 20 ring carbon atoms is, for instance, a group derived from any one of aromatic hydrocarbons selected from the group consisting of naphthalene, anthracene, acephenanthrylene, aceanthrylene, benzoanthracene, triphenylene, pyrene, chrysene, naphthacene, fluorene, phenanthrene, fluoranthene and benzofluoranthene.

In an exemplary embodiment, Ar₂ in the formula (21) is a substituted or unsubstituted anthryl group.

In an exemplary embodiment, Ar₂ in the formula (21) is a substituted or unsubstituted heterocyclic group having 5 to 40 ring carbon atoms.

In an exemplary embodiment, Ar₂ in the formula (21) is a substituted or unsubstituted group derived from the cyclic structure represented by the formula (20).

In an exemplary embodiment, Ar₂ in the formula (21) is a group represented by a formula (23) below.

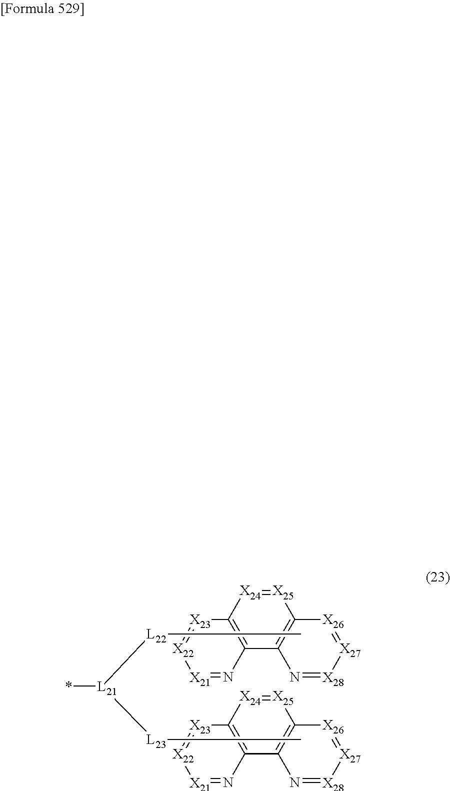

In the formula (23):
X₂₁ to X₂₈ are each independently a nitrogen atom, CR₂₁, a group represented by the formula (21), or a carbon atom bonded to L₂₂ or L₂₃;
L₂₁ is a linking group, and L₂₁ as the linking group is a substituted or unsubstituted trivalent linear, branched or cyclic aliphatic hydrocarbon group having 1 to 50 carbon atoms, a substituted or unsubstituted trivalent aromatic hydrocarbon group having 6 to 50 ring carbon atoms, or a substituted or unsubstituted trivalent heterocyclic group having 5 to 50 ring atoms; and
L₂₂ and L₂₃ are each independently a single bond or a linking group, and L₂₂ and L₂₃ as the linking group are each independently a substituted or unsubstituted divalent linear, branched or cyclic aliphatic hydrocarbon group having 1 to 50 carbon atoms, a substituted or unsubstituted arylene group having 6 to 50 ring carbon atoms, or a substituted or unsubstituted divalent heterocyclic group having 5 to 50 ring atoms.

In the formula of the phenanthroline compound:
when a plurality of X₂₁ are present, the plurality of X₂₁ are mutually the same or different;
when a plurality of X₂₂ are present, the plurality of X₂₂ are mutually the same or different;
when a plurality of X₂₃ are present, the plurality of X₂₃ are mutually the same or different;
when a plurality of X₂₄ are present, the plurality of X₂₄ are mutually the same or different;
when a plurality of X₂₅ are present, the plurality of X₂₅ are mutually the same or different;
when a plurality of X₂₆ are present, the plurality of X₂₆ are mutually the same or different;
when a plurality of X₂₇ are present, the plurality of X₂₇ are mutually the same or different; and
when a plurality of X₂₈ are present, the plurality of X₂₈ are mutually the same or different.

In an exemplary embodiment, X₂₁ to X₂₈ in the formula (23) are preferably each independently a nitrogen atom, CR₂₁, or a carbon atom bonded to L₂₂ or L₂₃, more preferably CR₂₁ or a carbon atom bonded to L₂₂ or L₂₃.

In an exemplary embodiment, the phenanthroline compound is a compound represented by a formula (24) below.

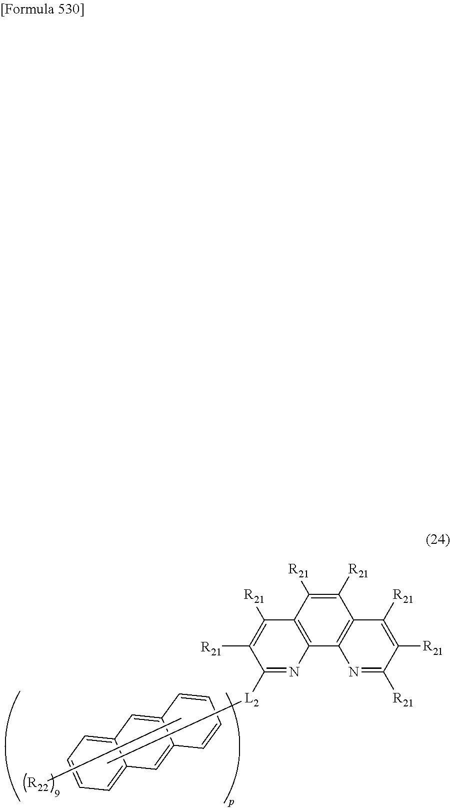

In the formula (24):
a plurality of R₂₁ each independently represent the same as R₂₁ in the formula (20);
a plurality of R₂₂ each independently represent the same as R₂₁ in the formula (20);
L₂ represents the same as L₂ in the formula (21);
p is 1, 2, 3, 4, or 5; and
a plurality of R₂₂ and L₂ are each bonded to a carbon atom at one of positions 1 to 10 of an anthracene ring.

In an exemplary embodiment, the phenanthroline compound is a compound represented by a formula (24A) below.

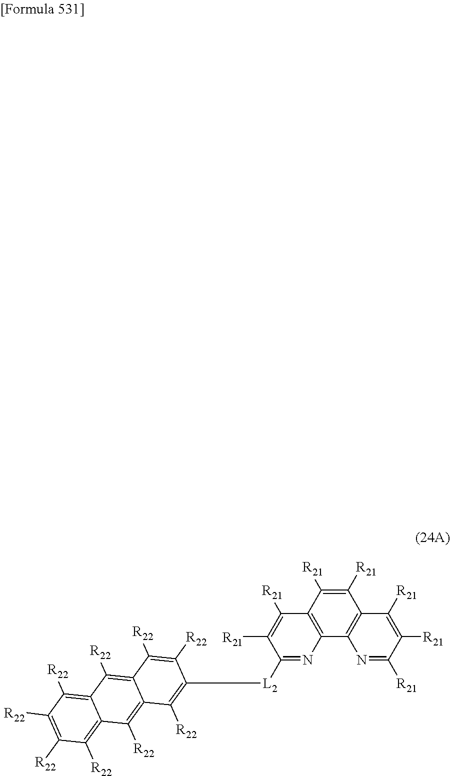

In the formula (24A):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, the phenanthroline compound is a compound represented by a formula (24B) below.

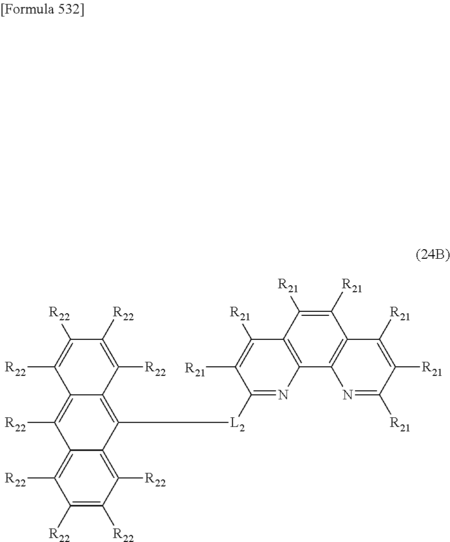

In the formula (24B):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, the phenanthroline compound is a compound represented by a formula (25) below.

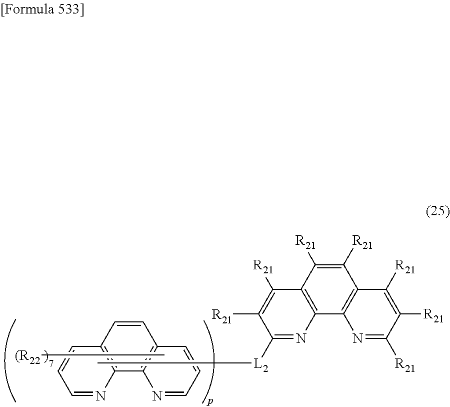

In the formula (25):
a plurality of R₂₁ each independently represent the same as R₂₁ in the formula (20);
a plurality of R₂₂ each independently represent the same as R₂₁ in the formula (20);
L₂ represents the same as L₂ in the formula (21);
p is 1, 2, 3, 4, or 5; and
a plurality of R₂₂ and L₂ are each bonded to a carbon atom at one of positions 2 to 9 of a phenanthroline ring.

In an exemplary embodiment, the phenanthroline compound is a compound represented by a formula (25A) below.

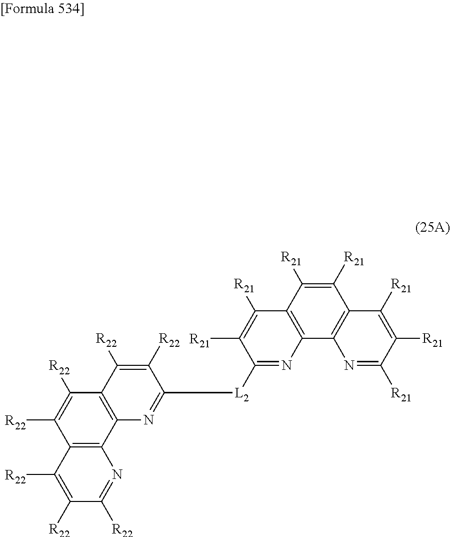

In the formula (25A):
a plurality of R₂₁ each independently represent the same as R₂₁ in the formula (20);
a plurality of R₂₂ each independently represent the same as R₂₁ in the formula (20); and
L₂ represents the same as L₂ in the formula (21).

In an exemplary embodiment, L₂ in the formulae (24), (24A), (24B), (25) and (25A) is a single bond, a substituted or unsubstituted arylene group having 6 to 50 ring carbon atoms, or a substituted or unsubstituted divalent heterocyclic group having 5 to 50 ring atoms.

In an exemplary embodiment, the phenanthroline compound is a compound represented by a formula (25B) below.

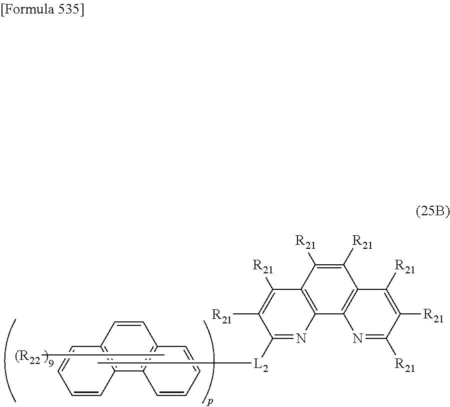

In the formula (25B):
a plurality of R₂₁ each independently represent the same as R₂₁ in the formula (20);
a plurality of R₂₂ each independently represent the same as R₂₁ in the formula (20);
L₃ is a linking group, and L₃ as the linking group is a substituted or unsubstituted polyvalent linear, branched or cyclic aliphatic hydrocarbon group having 1 to 50 carbon atoms, a substituted or unsubstituted polyvalent amino group, a substituted or unsubstituted polyvalent aromatic hydrocarbon ring group having 6 to 50 ring carbon atoms, a substituted or unsubstituted polyvalent heterocyclic group having 5 to 50 ring atoms, or a polyvalent multiple linking group provided by bonding two or three groups selected from the polyvalent aromatic hydrocarbon ring group and the polyvalent heterocyclic group;
the aromatic hydrocarbon ring group and the heterocyclic group forming L₃ as the multiple linking group are mutually the same or different, and adjacent ones thereof are mutually bonded to form a ring, or not mutually bonded;
p is 1, 2, 3, 4, or 5; and
a plurality of R₂₂ and L₃ are each bonded to a carbon atom at one of positions 1 to 10 of a phenanthrene ring.

In an exemplary embodiment, the phenanthroline compound is a compound represented by a formula (25C) below.

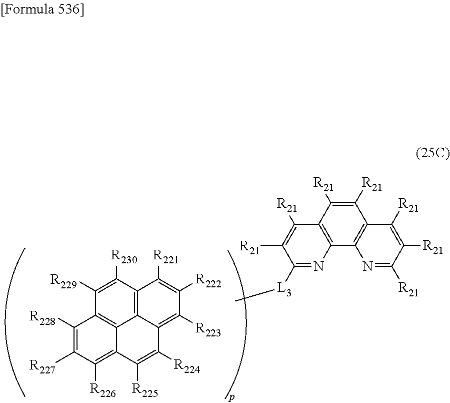

In the formula (25C):
a plurality of R₂₁ each independently represent the same as R₂₁ in the formula (20);
one of R₂₂₁ to R₂₃₀ is a single bond with L₃, and R₂₂₁ to R₂₃₀ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

In an exemplary embodiment, the phenanthroline compound is a compound represented by a formula (25D) below.

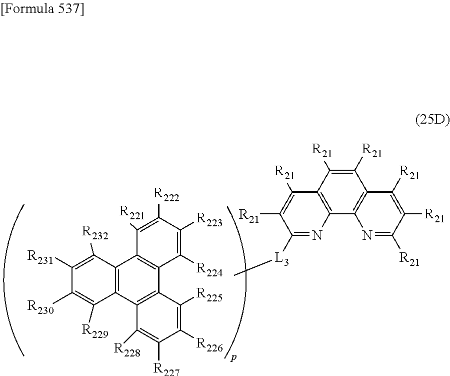

In the formula (25D):
a plurality of R₂₁ each independently represent the same as R₂₁ in the formula (20);
one of R₂₂₁ to R₂₃₂ is a single bond with L₃, and R₂₂₁ to R₂₃₂ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

In an exemplary embodiment, the phenanthroline compound is a compound represented by a formula (25E) below.

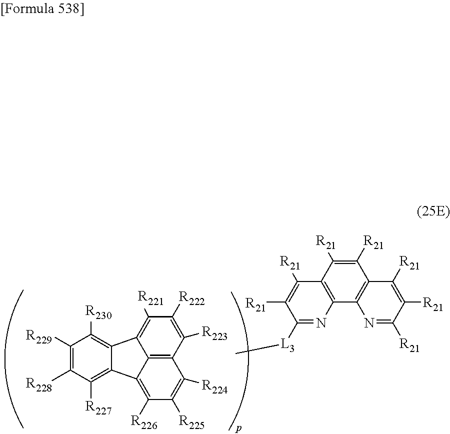

In the formula (25E):
a plurality of R₂₁ each independently represent the same as R₂₁ in the formula (20);
one of R₂₂₁ to R₂₃₀ is a single bond with L₃, and R₂₂₁ to R₂₃₀ not being the single bond with L₃ each independently represent the same as R₂₁ in the formula (20);
L₃ is a linking group, and L₃ as the linking group represents the same as L₃ as the linking group in the formula (25B); and
p is 1, 2, 3, 4, or 5.

Also preferably, L₃ in the formulae (25B), (25C), (25D) and (25E) are each a substituted or unsubstituted arylene group having 6 to 50 ring carbon atoms, or a substituted or unsubstituted divalent heterocyclic group having 5 to 50 ring atoms.

### Method for Producing Phenanthroline Compound

The phenanthroline compound can be produced by a known method. The phenanthroline compound also can be produced based on a known method through a known alternative reaction using a known material(s) tailored for the target compound.

### Azine Derivative

In an exemplary arrangement of the organic EL device of each exemplary embodiment, at least one electron transporting layer in the electron transporting zone contains an azine derivative as a nitrogen-containing compound.

In an exemplary arrangement of the organic EL device of each exemplary embodiment, the azine derivative contained in the electron transporting layer is a compound represented by a formula (E42) below.

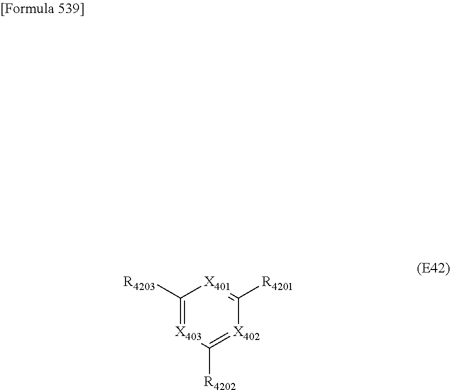

In the formula (E42):
X₄₀₁ to X₄₀₃ are each independently CR₄₂₀₄ or a nitrogen atom;
at least one of X₄₀₁ to X₄₀₃ is a nitrogen atom;
when two or more R₄₂₀₄ are present, the two or more R₄₂₀₄ are mutually the same or different; and
R₄₂₀₁ to R₄₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₉₀₁ to R₉₀₇ in the azine derivative are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device of each exemplary embodiment, the azine derivative contained in the electron transporting layer is a compound represented by a formula (E421) below.

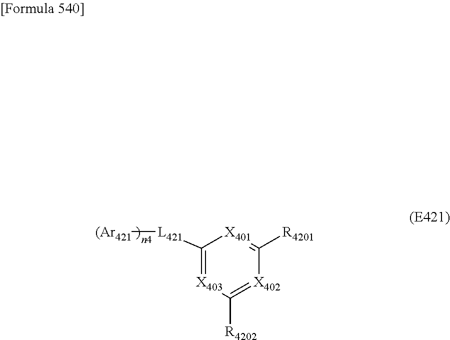

In the formula (E421):
X₄₀₁ to X₄₀₃ and R₄₂₀₁ and R₄₂₀₂ each independently represent the same as X₄₀₁ to X₄₀₃ and R₄₂₀₁ and R₄₂₀₂ in the formula (E42);
n₄ is 1, 2, or 3;
L₄₂₁ is a single bond, a substituted or unsubstituted arylene group having 6 to 50 ring carbon atoms, a trivalent group or tetravalent group derived from the arylene group, a substituted or unsubstituted divalent heterocyclic group having 5 to 50 ring atoms, a trivalent group or tetravalent group derived from the divalent heterocyclic group, or a divalent group formed by bonding two groups selected from the group consisting of a substituted or unsubstituted arylene group having 6 to 50 ring carbon atoms, and a substituted or unsubstituted divalent heterocyclic group having 5 to 50 ring atoms, or a trivalent group or tetravalent group derived from the divalent group;
Ar₄₂₁ is a substituted or unsubstituted aryl group having 6 to 50 ring carbon atoms, or a substituted or unsubstituted heterocyclic group having 5 to 50 ring atoms;
when n₄ is 2 or 3, Ar₄₂₁ are mutually the same or different; and
when n₄ is 2 or 3, L₄₂₁ is not a single bond.

In an exemplary arrangement of the organic EL device of each exemplary embodiment, the azine derivative contained in the electron transporting layer is a compound represented by each of a formula (E422) and a formula (E423) below.

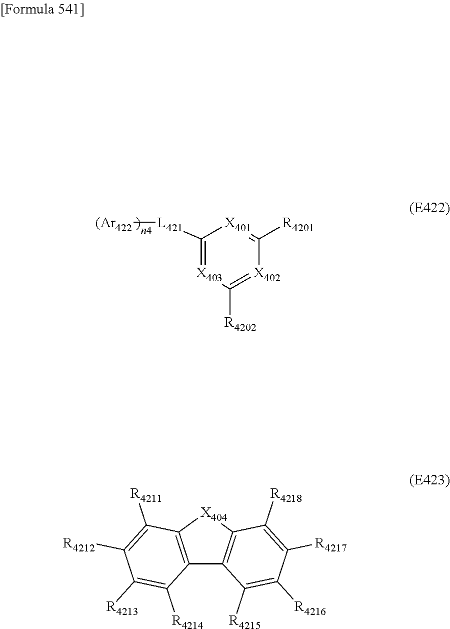

In the formula (E422):
n₄, X₄₀₁ to X₄₀₃, R₄₂₀₁ and R₄₂₀₂ each independently represent the same as n₄, X₄₀₁ to X₄₀₃, R₄₂₀₁ and R₄₂₀₂ in the formula (E42);
L₄₂₁ represents the same as L₄₂₁ in the formula (E421); and
Ar₄₂₂ is a group represented by the formula (E423).

In the formula (E423):
X₄₀₄ is an oxygen atom, a sulfur atom, N(R₄₂₂₁) or C(R₄₂₂₂)(R₄₂₂₃); one of R₄₂₁₁ to R₄₂₁₈ and R₄₂₂₁ to R₄₂₂₃ is a single bond with L₄₂₁;
at least one combination of adjacent two or more of R₄₂₁₁ to R₄₂₁₈ not being the single bond with L₄₂₁ are mutually bonded to form a substituted or unsubstituted monocyclic ring, mutually bonded to form a substituted or unsubstituted fused ring, or not mutually bonded;
a combination of R₄₂₂₂ and R₄₂₂₃ not being the single bond with L₄₂₁ are mutually bonded to form a substituted or unsubstituted monocyclic ring, mutually bonded to form a substituted or unsubstituted fused ring, or not mutually bonded;
R₄₂₁₁ to R₄₂₁₈ not being the single bond with L₄₂₁ and forming neither the substituted or unsubstituted monocyclic ring nor the substituted or unsubstituted fused ring, R₄₂₂₁, and R₄₂₂₂ and R₄₂₂₃ not being the single bond with L₄₂₁ and forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
when n₄ is 2 or 3,
a plurality of X₄₀₄ are mutually the same or different;
a plurality of R₄₂₁₁ are mutually the same or different;
a plurality of R₄₂₁₂ are mutually the same or different;
a plurality of R₄₂₁₃ are mutually the same or different;
a plurality of R₄₂₁₄ are mutually the same or different;
a plurality of R₄₂₁₅ are mutually the same or different;
a plurality of R₄₂₁₆ are mutually the same or different;
a plurality of R₄₂₁₇ are mutually the same or different; and
a plurality of R₄₂₁₈ are mutually the same or different.

In the formulae (E422) and (E423), R₉₀₁ to R₉₀₇ each independently represent the same as R₉₀₁ to R₉₀₇ in the azine derivative.

When n₄ is 1 in the formula (E421), for instance, the group represented by (Ar₄₂₁)ₙ₄-L₄₂₁-* in the formula (E421) is represented by a formula (E421-1) below. In this arrangement, L₄₂₁ is a divalent linking group. * represents a bonding position with a six-membered ring in the formula (E421).

When n₄ is 2 in the formula (E421), the group represented by (Ar₄₂₁)ₙ₄-L₄₂₁-* in the formula (E421) is represented by a formula (E421-2) below. Ar₄₂₁ are mutually the same or different. In this arrangement, L₄₂₁ is a trivalent linking group.

When n₄ is 3 in the formula (E421), the group represented by (Ar₄₂₁) n₄-L₄₂₁-* in the formula (E421) is represented by a formula (E421-3) below. Ar₄₂₁ are mutually the same or different. In this arrangement, L₄₂₁ is a tetravalent linking group. The same applies to a group represented by (Ar₄₂₂)ₙ₄-L₄₂₁-* in the formula (E422).

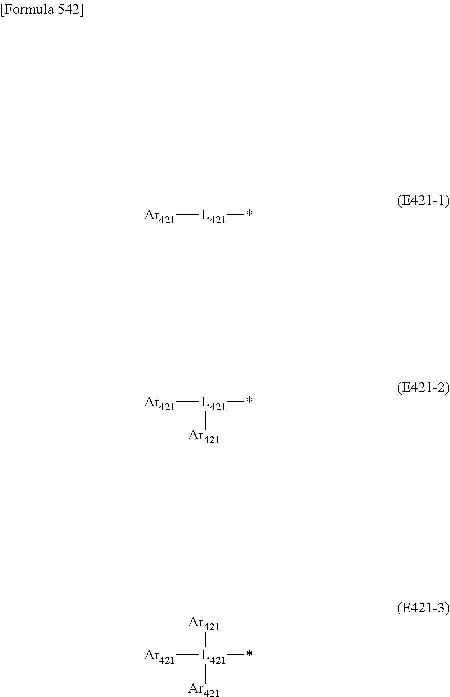

In the formulae (E421-1), (E421-2) and (E421-3), * each represent a bonding position with a six-membered ring in the formula (E421).

In the formulae (E421) and (E422), L₄₂₁ as the linking group is preferably a divalent or trivalent group derived from any of benzene, biphenyl, terphenyl, naphthalene and phenanthrene.

In an exemplary arrangement of the organic EL device of each exemplary embodiment, a substituent for “a substituted or unsubstituted” group in the azine derivative is at least one group selected from the group consisting of an alkyl group having 1 to 18 carbon atoms, aryl group having 6 to 18 ring carbon atoms, and heterocyclic group having 5 to 18 ring atoms.

In an exemplary arrangement of the organic EL device of each exemplary embodiment, a substituent for “the substituted or unsubstituted” group in the azine derivative is an alkyl group having 1 to 5 carbon atoms.

In an exemplary arrangement of the organic EL device of each exemplary embodiment, the groups specified to be “substituted or unsubstituted” in the azine derivative are each an “unsubstituted” group.

### Benzimidazole Derivative

In an exemplary arrangement of the organic EL device of each exemplary embodiment, at least one electron transporting layer in the electron transporting zone contains a benzimidazole derivative as a nitrogen-containing compound.

In an exemplary arrangement of the organic EL device of each exemplary embodiment, the benzimidazole derivative contained in the electron transporting layer is a compound represented by a formula (E41) below.

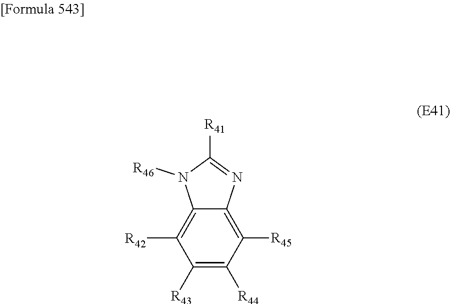

In the formula (E41):
at least one combination of adjacent two or more of R₄₁ to R₄₆ are mutually bonded to form a substituted or unsubstituted monocyclic ring, mutually bonded to form a substituted or unsubstituted fused ring, or not mutually bonded; and
R₄₁ to R₄₆ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (E41), R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of Roos are present, the plurality of R₉₀₆ are mutually the same or different; and
when a plurality of R₉₀₇ are present, the plurality of R₉₀₇ are mutually the same or different.

In an exemplary arrangement of the organic EL device according to each exemplary embodiment, the benzimidazole derivative contained in the electron transporting layer is a compound represented by a formula (E41A), (E41B), (E41C), (E41D) or (E41E) below.

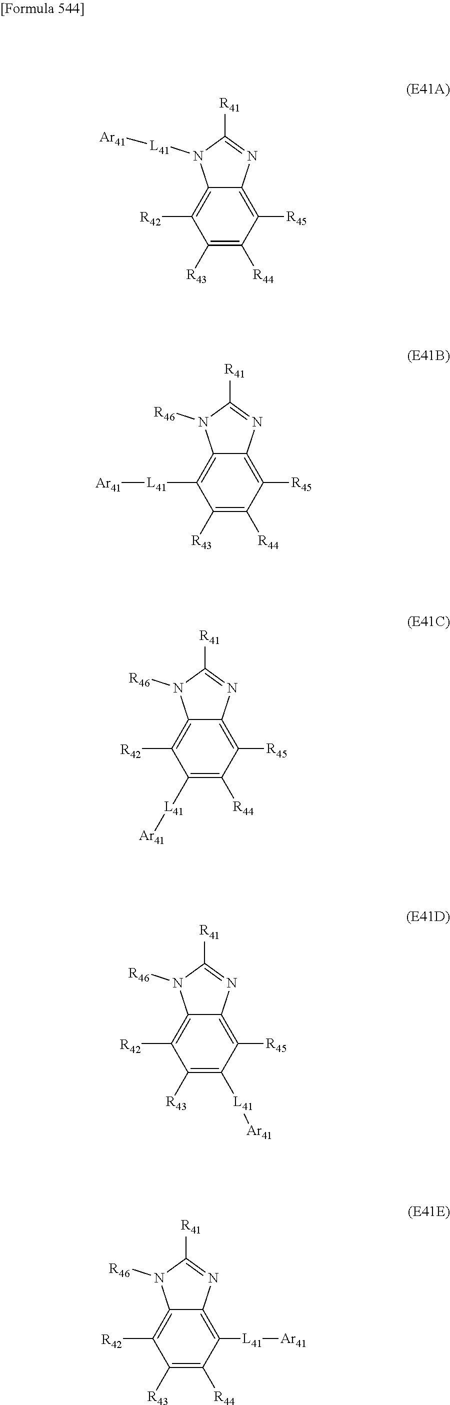

In the formulae (E41A), (E41B), (E41C), (E41D) and (E41E):
R₄₁ to R₄₆ each independently represent the same as R₄₁ to R₄₆ in the formula
(E41);
L₄₁ is a single bond, a substituted or unsubstituted arylene group having 6 to 50 ring carbon atoms, or a substituted or unsubstituted divalent heterocyclic group having 5 to 50 ring atoms;
Ar₄₁ is a substituted or unsubstituted alkyl group having 1 to 50 carbon atoms, a group represented by —O—(R₉₀₄), a substituted or unsubstituted aryl group having 6 to 50 ring carbon atoms, or a substituted or unsubstituted heterocyclic group having 5 to 50 ring atoms; and
R₉₀₄ represents the same as R₉₀₄ in the benzimidazole derivative.

In an exemplary arrangement of the organic EL device of the exemplary embodiment, Ar₄₁ in the formulae (E41A), (E41B), (E41C), (E41D), and (E41E) is a group represented by a formula (E412) below.

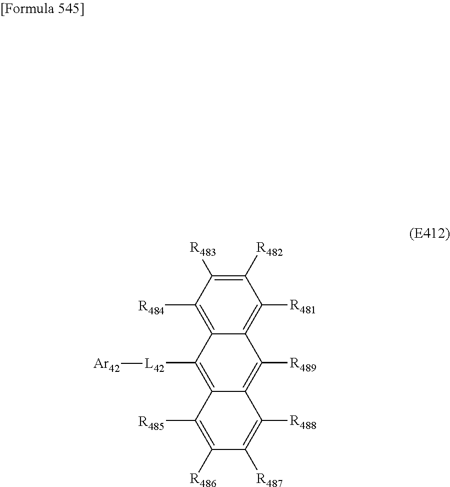

In the formula (E412):
one of R₄₈₁ to R₄₈₉ is a single bond with L₄₁;
at least one combination of adjacent two or more of R₄₈₁ to R₄₈₉ not being the single bond with L₄₁ are mutually bonded to form a substituted or unsubstituted monocyclic ring, mutually bonded to form a substituted or unsubstituted fused ring, or not mutually bonded;
R₄₈₁ to R₄₈₉ not being the single bond with L₄₁, not forming the substituted or unsubstituted monocyclic ring, and not forming the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
L₄₂ is each independently a single bond, a substituted or unsubstituted arylene group having 6 to 50 ring carbon atoms, or a substituted or unsubstituted divalent heterocyclic group having 5 to 50 ring atoms; and
Ar₄₂ is each independently a substituted or unsubstituted aryl group having 6 to 50 ring carbon atoms, or a substituted or unsubstituted heterocyclic group having 5 to 50 ring atoms.

In the formula (E412), R₉₀₁ to R₉₀₇ each independently represent the same 5 as R₉₀₁ to R₉₀₇ in the benzimidazole derivative.

In the formulae (E41A), (E41B), (E41C), (E41D) and (E41E): L₄₁ is preferably a substituted or unsubstituted phenylene group, a substituted or unsubstituted biphenylene group, a substituted or unsubstituted naphthylene group, a substituted or unsubstituted pyridinylene group, or a substituted or unsubstituted 9,9′-spirobifluorenylene group.

In the formulae (E41A), (E41B), (E41C), (E41D) and (E41E): L₄₁ is preferably an unsubstituted phenylene group, an unsubstituted biphenylene group, an unsubstituted naphthylene group, an unsubstituted pyridinylene group, or an unsubstituted 9,9′-spirobifluorenylene group.

In the formulae (E41A), (E41B), (E41C), (E41D) and (E41E): Ar₄₁ is preferably a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted fluoranthenyl group, a substituted or unsubstituted pyrenyl group, or a substituted or unsubstituted fluorenyl group.

In the formulae (E41A), (E41B), (E41C), (E41D) and (E41E): Ar₄₁ is preferably an unsubstituted phenyl group, an unsubstituted biphenyl group, an unsubstituted terphenyl group, an unsubstituted naphthyl group, an unsubstituted phenanthryl group, an unsubstituted fluoranthenyl group, an unsubstituted pyrenyl group, or an unsubstituted 9,9-dimethylfluorenyl group.

In an exemplary arrangement of the organic EL device of each exemplary embodiment, the benzimidazole derivative is a compound represented by a formula (E413) below.

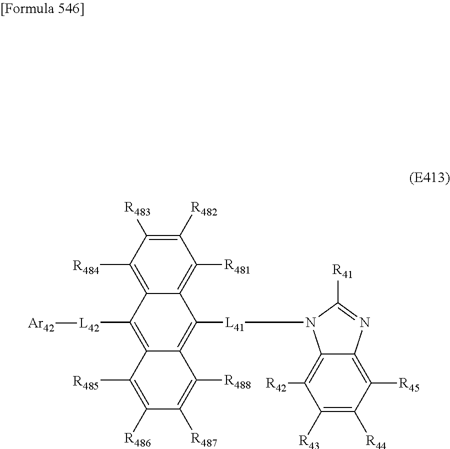

In the formula (E413):
R₄₁ to R₄₅ and L₄₁ each independently represent the same as R₄₁ to R₄₅ and L₄₁ in the formula (E41A); and
R₄₈₁ to R₄₈₈, L₄₂ and Ar₄₂ each independently represent the same as R₄₈₁ to R₄₈₈, L₄₂ and Ar₄₂ in the formula (E412).

In an exemplary arrangement of the organic EL device of each exemplary embodiment, the groups specified to be “substituted or unsubstituted” in the benzimidazole derivative are each an “unsubstituted” group.

### Specific Examples of Nitrogen-Containing Compound

Specific examples of the nitrogen-containing compound include the following compounds. However, the invention is by no means limited to the specifically listed nitrogen-containing compounds.

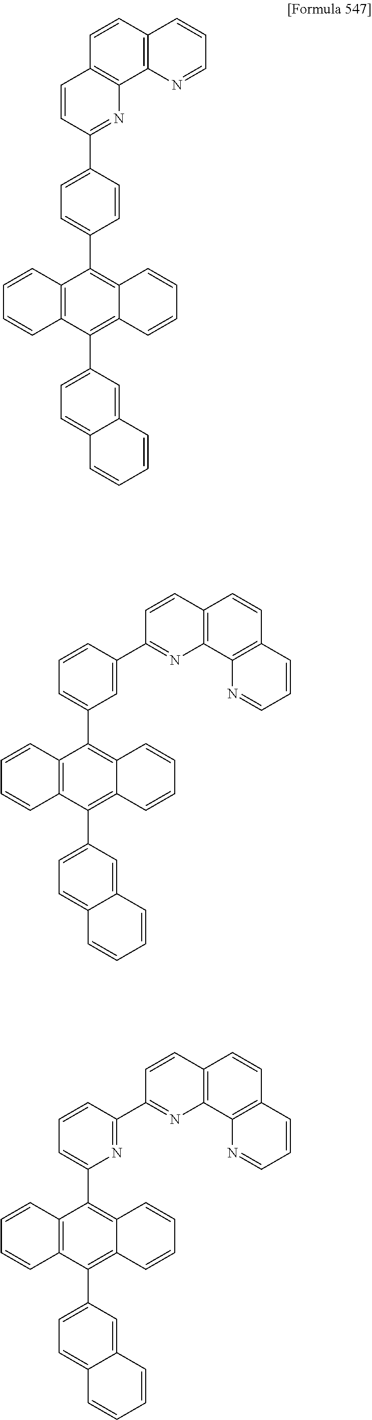 [SMILES: c1ccc2cc(-c3c4ccccc4c(-c4ccc(-c5ccc6ccc7cccnc7c6n5)cc4)c4ccccc34)ccc2c1 ; c1cc(-c2ccc3ccc4cccnc4c3n2)cc(-c2c3ccccc3c(-c3ccc4ccccc4c3)c3ccccc23)c1 ; c1cc(-c2ccc3ccc4cccnc4c3n2)nc(-c2c3ccccc3c(-c3ccc4ccccc4c3)c3ccccc23)c1]

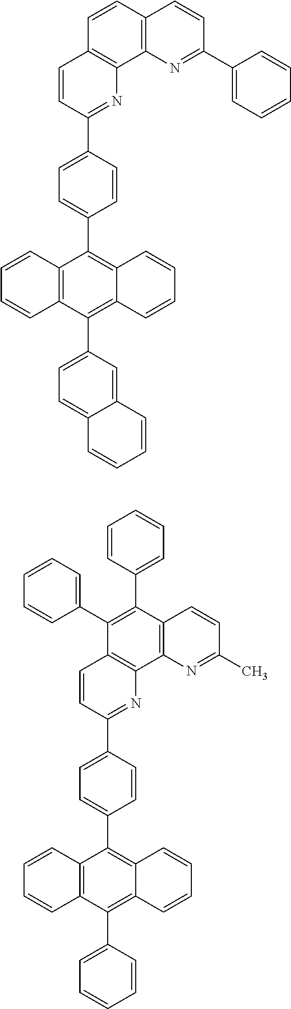 [SMILES: c1ccc(-c2ccc3ccc4ccc(-c5ccc(-c6c7ccccc7c(-c7ccc8ccccc8c7)c7ccccc67)cc5)nc4c3n2)cc1 ; Cc1ccc2c(-c3ccccc3)c(-c3ccccc3)c3ccc(-c4ccc(-c5c6ccccc6c(-c6ccccc6)c6ccccc56)cc4)nc3c2n1]

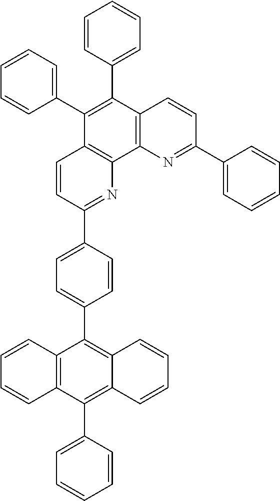 [SMILES: c1ccc(-c2ccc3c(-c4ccccc4)c(-c4ccccc4)c4ccc(-c5ccc(-c6c7ccccc7c(-c7ccccc7)c7ccccc67)cc5)nc4c3n2)cc1]

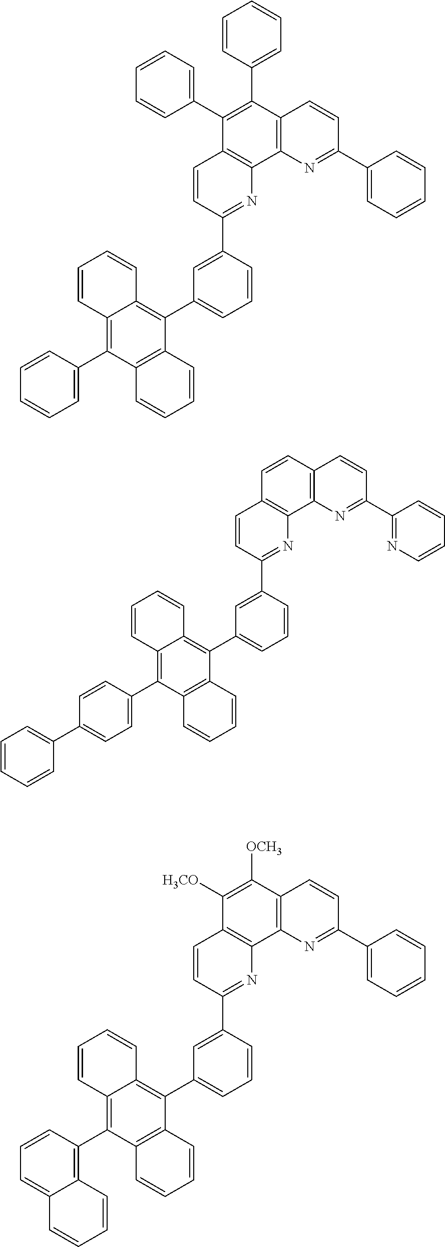 [SMILES: c1ccc(-c2ccc3c(-c4ccccc4)c(-c4ccccc4)c4ccc(-c5cccc(-c6c7ccccc7c(-c7ccccc7)c7ccccc67)c5)nc4c3n2)cc1 ; c1ccc(-c2ccc(-c3c4ccccc4c(-c4cccc(-c5ccc6ccc7ccc(-c8ccccn8)nc7c6n5)c4)c4ccccc34)cc2)cc1 ; COc1c(OC)c2ccc(-c3cccc(-c4c5ccccc5c(-c5cccc6ccccc56)c5ccccc45)c3)nc2c2nc(-c3ccccc3)ccc12]

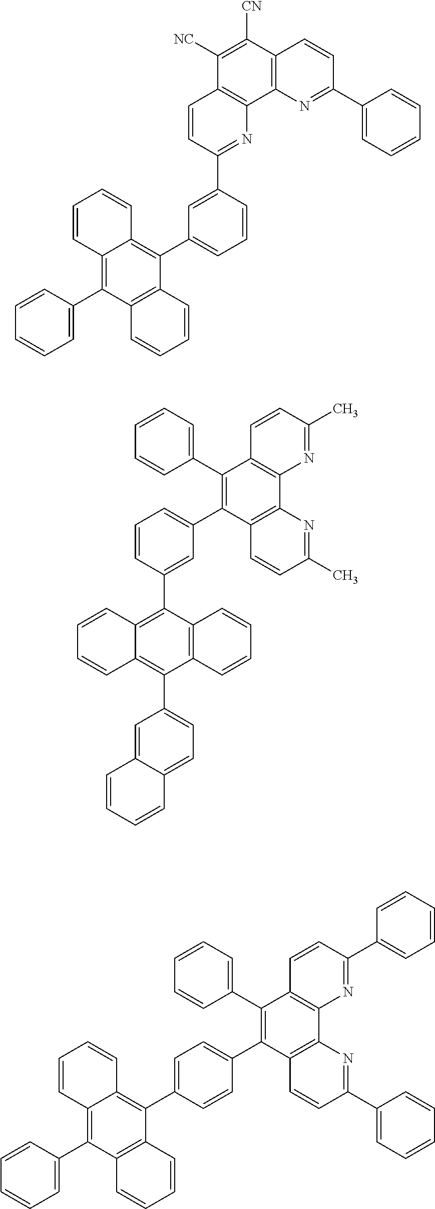 [SMILES: N#Cc1c(C#N)c2ccc(-c3cccc(-c4c5ccccc5c(-c5ccccc5)c5ccccc45)c3)nc2c2nc(-c3ccccc3)ccc12 ; Cc1ccc2c(-c3ccccc3)c(-c3cccc(-c4c5ccccc5c(-c5ccc6ccccc6c5)c5ccccc45)c3)c3ccc(C)nc3c2n1 ; c1ccc(-c2ccc3c(-c4ccccc4)c(-c4ccc(-c5c6ccccc6c(-c6ccccc6)c6ccccc56)cc4)c4ccc(-c5ccccc5)nc4c3n2)cc1]

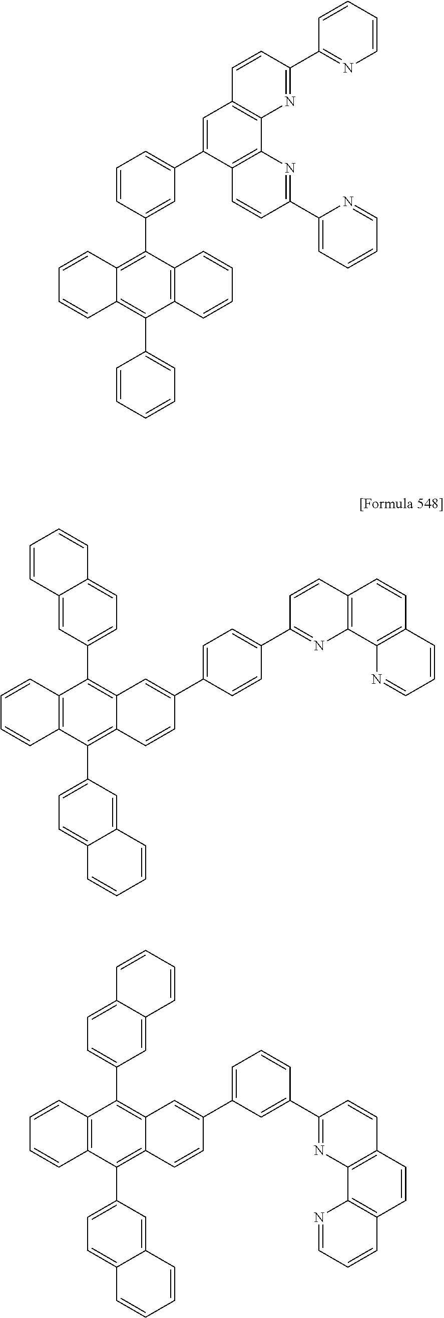 [SMILES: c1ccc(-c2c3ccccc3c(-c3cccc(-c4cc5ccc(-c6ccccn6)nc5c5nc(-c6ccccn6)ccc45)c3)c3ccccc23)cc1 ; c1ccc2cc(-c3c4ccccc4c(-c4ccc5ccccc5c4)c4cc(-c5ccc(-c6ccc7ccc8cccnc8c7n6)cc5)ccc34)ccc2c1 ; c1cc(-c2ccc3c(-c4ccc5ccccc5c4)c4ccccc4c(-c4ccc5ccccc5c4)c3c2)cc(-c2ccc3ccc4cccnc4c3n2)c1]

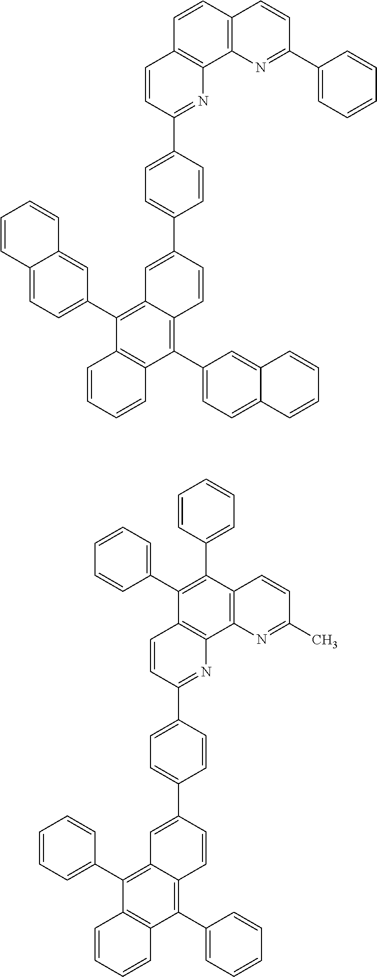 [SMILES: c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1 ; Cc1ccc2c(-c3ccccc3)c(-c3ccccc3)c3ccc(-c4ccc(-c5ccc6c(-c7ccccc7)c7ccccc7c(-c7ccccc7)c6c5)cc4)nc3c2n1]

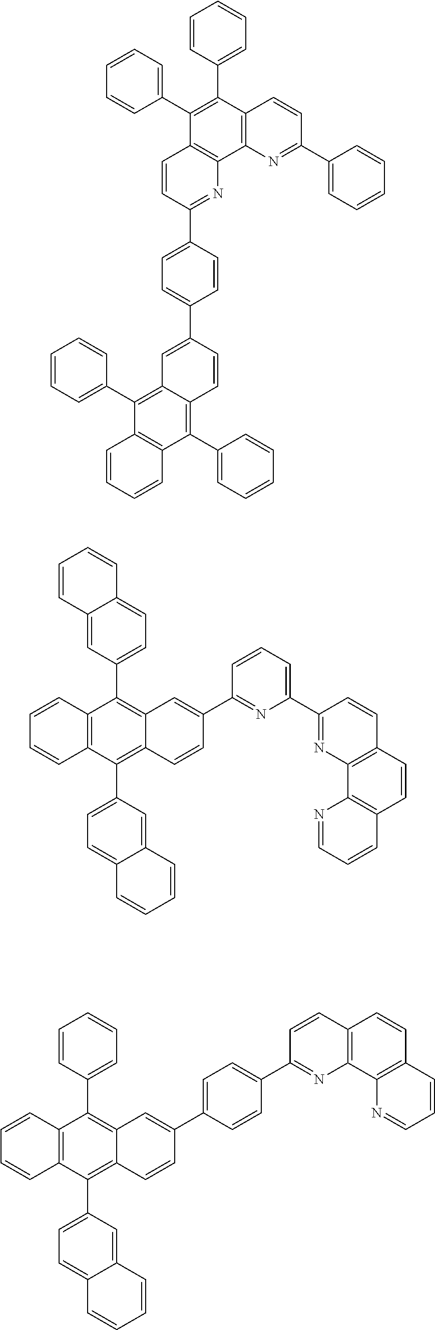 [SMILES: c1ccc(-c2ccc3c(-c4ccccc4)c(-c4ccccc4)c4ccc(-c5ccc(-c6ccc7c(-c8ccccc8)c8ccccc8c(-c8ccccc8)c7c6)cc5)nc4c3n2)cc1 ; c1cc(-c2ccc3c(-c4ccc5ccccc5c4)c4ccccc4c(-c4ccc5ccccc5c4)c3c2)nc(-c2ccc3ccc4cccnc4c3n2)c1 ; c1ccc(-c2c3ccccc3c(-c3ccc4ccccc4c3)c3ccc(-c4ccc(-c5ccc6ccc7cccnc7c6n5)cc4)cc23)cc1]

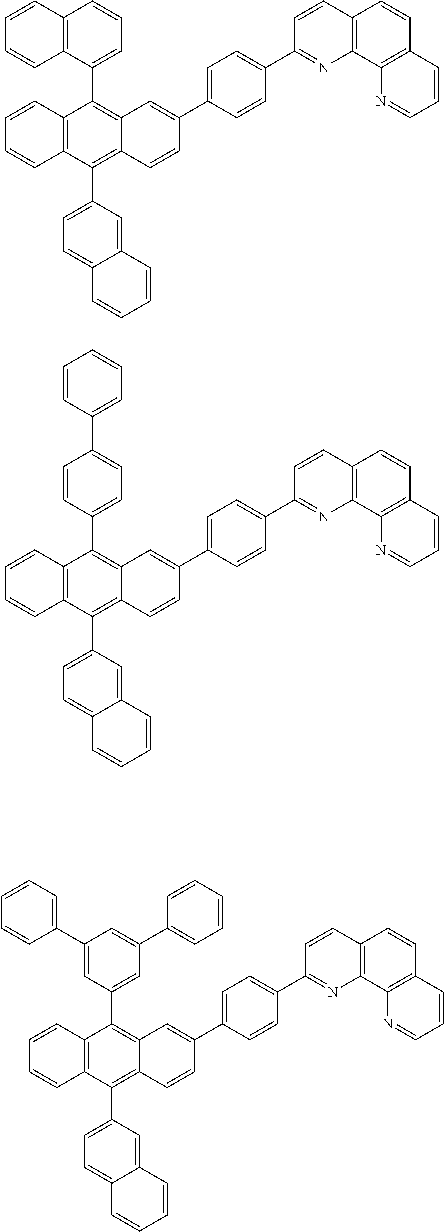

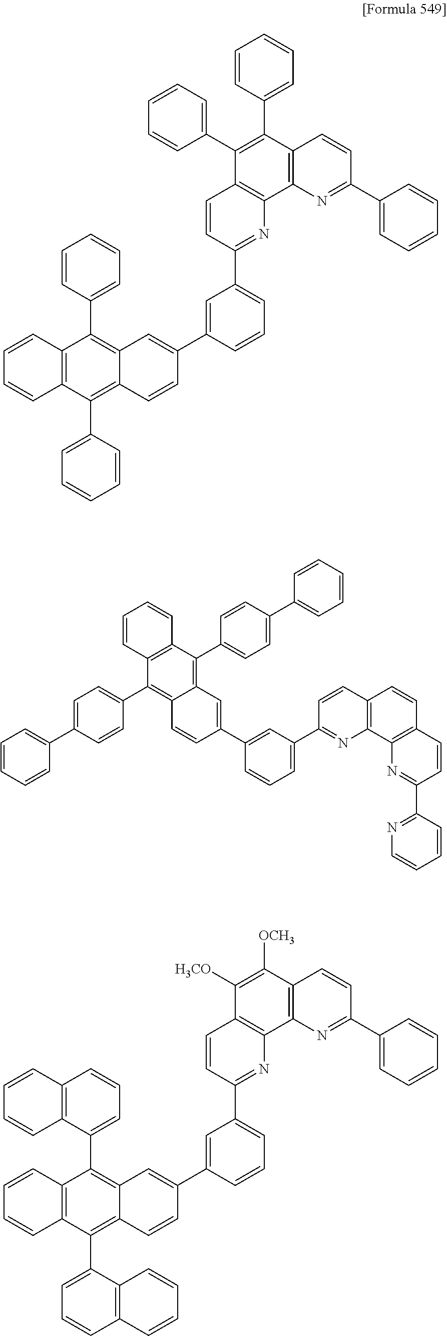 [SMILES: c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1 ; c1ccc(-c2ccc(-c3c4ccccc4c(-c4ccc(-c5ccccc5)cc4)c4cc(-c5cccc(-c6ccc7ccc8ccc(-c9ccccn9)nc8c7n6)c5)ccc34)cc2)cc1 ; COc1c(OC)c2ccc(-c3cccc(-c4ccc5c(-c6cccc7ccccc67)c6ccccc6c(-c6cccc7ccccc67)c5c4)c3)nc2c2nc(-c3ccccc3)ccc12]

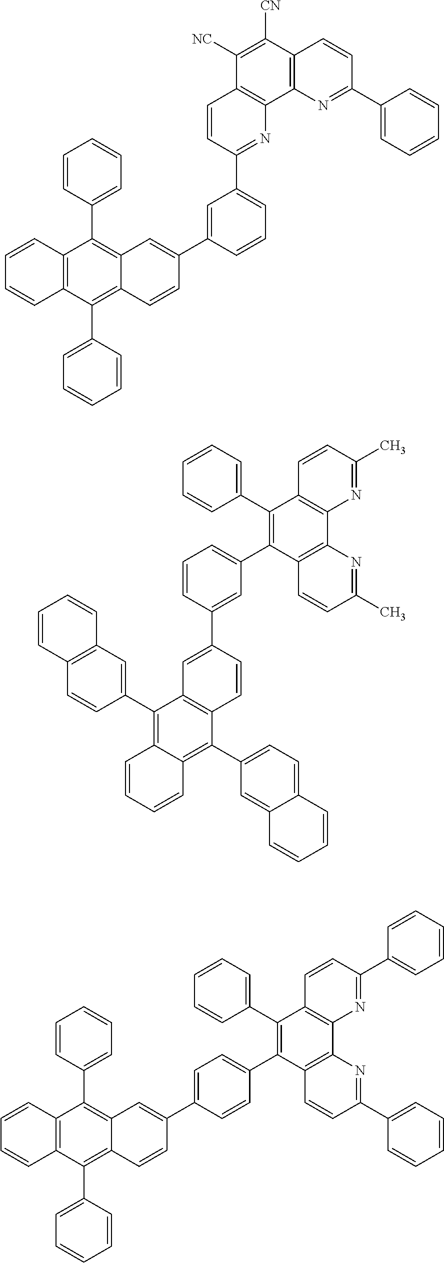 [SMILES: N#Cc1c(C#N)c2ccc(-c3cccc(-c4ccc5c(-c6ccccc6)c6ccccc6c(-c6ccccc6)c5c4)c3)nc2c2nc(-c3ccccc3)ccc12 ; Cc1ccc2c(-c3ccccc3)c(-c3cccc(-c4ccc5c(-c6ccc7ccccc7c6)c6ccccc6c(-c6ccc7ccccc7c6)c5c4)c3)c3ccc(C)nc3c2n1 ; c1ccc(-c2ccc3c(-c4ccccc4)c(-c4ccc(-c5ccc6c(-c7ccccc7)c7ccccc7c(-c7ccccc7)c6c5)cc4)c4ccc(-c5ccccc5)nc4c3n2)cc1]

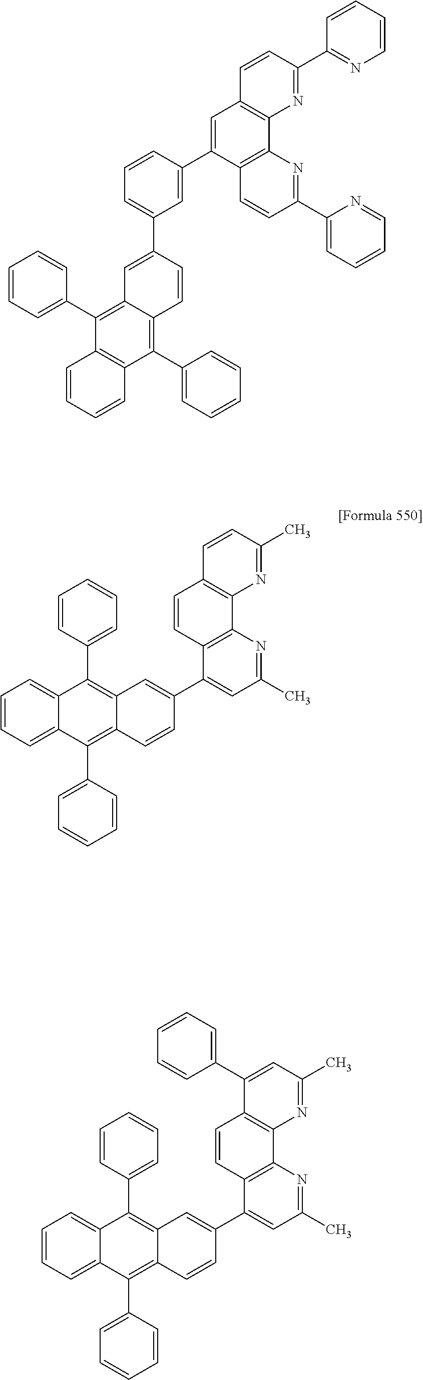 [SMILES: c1ccc(-c2c3ccccc3c(-c3ccccc3)c3cc(-c4cccc(-c5cc6ccc(-c7ccccn7)nc6c6nc(-c7ccccn7)ccc56)c4)ccc23)cc1 ; Cc1ccc2ccc3c(-c4ccc5c(-c6ccccc6)c6ccccc6c(-c6ccccc6)c5c4)cc(C)nc3c2n1 ; Cc1cc(-c2ccccc2)c2ccc3c(-c4ccc5c(-c6ccccc6)c6ccccc6c(-c6ccccc6)c5c4)cc(C)nc3c2n1]

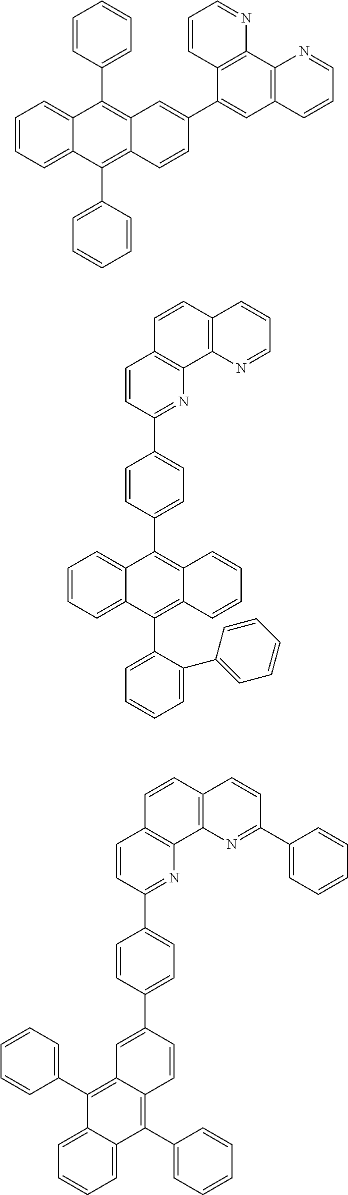 [SMILES: c1ccc(-c2c3ccccc3c(-c3ccccc3)c3cc(-c4cc5cccnc5c5ncccc45)ccc23)cc1 ; c1ccc(-c2ccccc2-c2c3ccccc3c(-c3ccc(-c4ccc5ccc6cccnc6c5n4)cc3)c3ccccc23)cc1 ; c1ccc(-c2ccc3ccc4ccc(-c5ccc(-c6ccc7c(-c8ccccc8)c8ccccc8c(-c8ccccc8)c7c6)cc5)nc4c3n2)cc1]

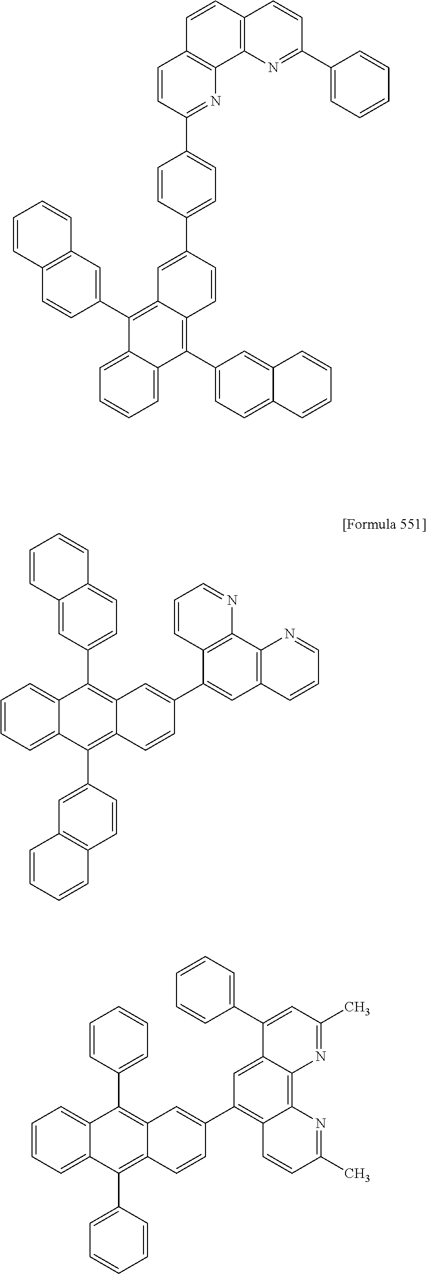 [SMILES: c1ccc(-c2ccc3ccc4ccc(-c5ccc(-c6ccc7c(-c8ccc9ccccc9c8)c8ccccc8c(-c8ccc9ccccc9c8)c7c6)cc5)nc4c3n2)cc1 ; c1ccc2cc(-c3c4ccccc4c(-c4ccc5ccccc5c4)c4cc(-c5cc6cccnc6c6ncccc56)ccc34)ccc2c1 ; Cc1ccc2c(-c3ccc4c(-c5ccccc5)c5ccccc5c(-c5ccccc5)c4c3)cc3c(-c4ccccc4)cc(C)nc3c2n1]

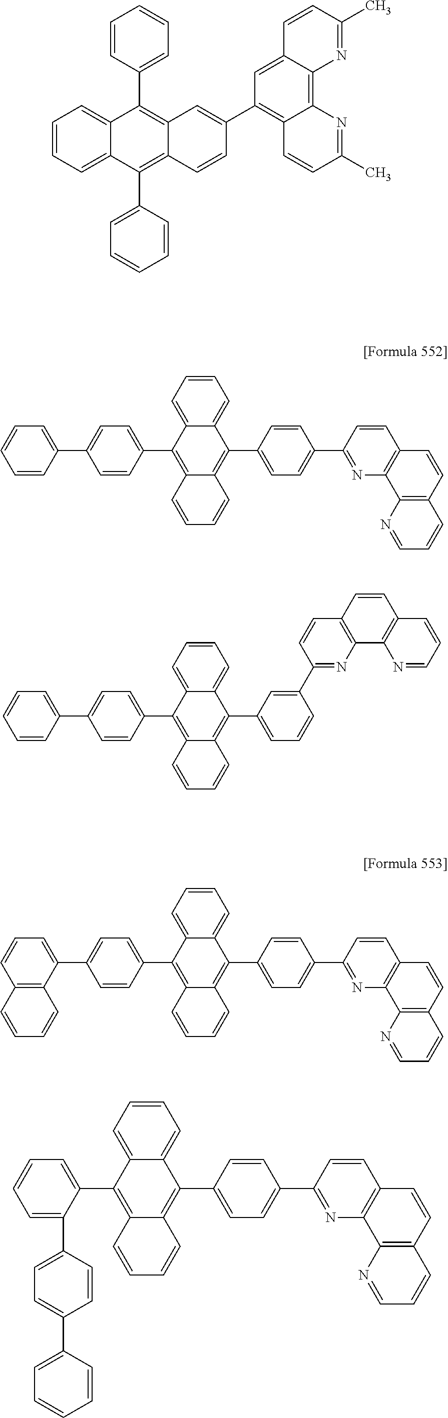 [SMILES: Cc1ccc2cc(-c3ccc4c(-c5ccccc5)c5ccccc5c(-c5ccccc5)c4c3)c3ccc(C)nc3c2n1 ; c1ccc(-c2ccc(-c3c4ccccc4c(-c4ccc(-c5ccc6ccc7cccnc7c6n5)cc4)c4ccccc34)cc2)cc1 ; c1ccc(-c2ccc(-c3c4ccccc4c(-c4cccc(-c5ccc6ccc7cccnc7c6n5)c4)c4ccccc34)cc2)cc1 ; c1ccc2c(-c3ccc(-c4c5ccccc5c(-c5ccc(-c6ccc7ccc8cccnc8c7n6)cc5)c5ccccc45)cc3)cccc2c1 ; c1ccc(-c2ccc(-c3ccccc3-c3c4ccccc4c(-c4ccc(-c5ccc6ccc7cccnc7c6n5)cc4)c4ccccc34)cc2)cc1]

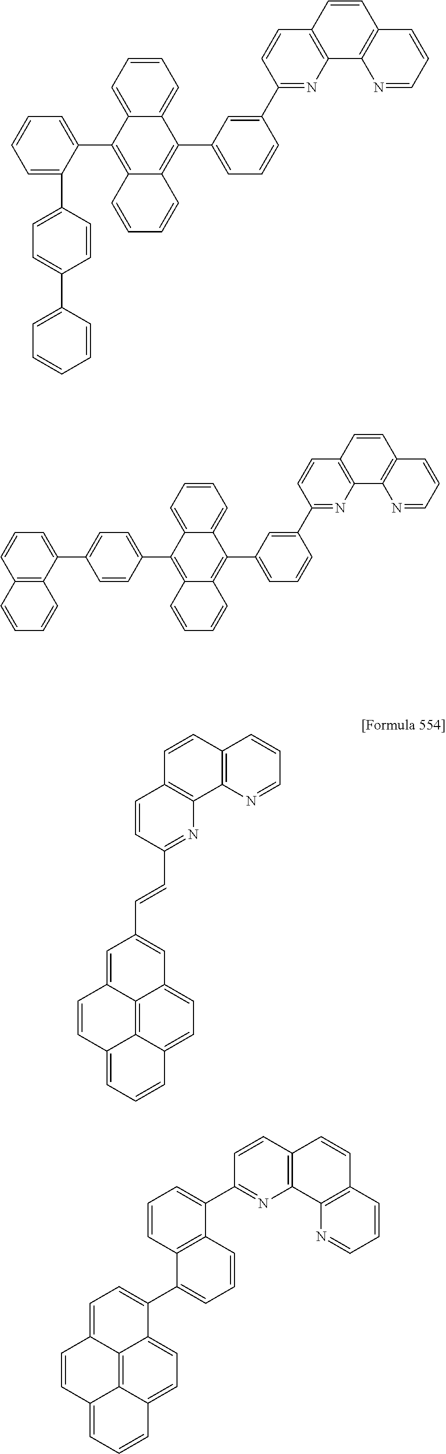 [SMILES: c1ccc(-c2ccc(-c3ccccc3-c3c4ccccc4c(-c4cccc(-c5ccc6ccc7cccnc7c6n5)c4)c4ccccc34)cc2)cc1 ; c1cc(-c2ccc3ccc4cccnc4c3n2)cc(-c2c3ccccc3c(-c3ccc(-c4cccc5ccccc45)cc3)c3ccccc23)c1 ; C(=C\c1ccc2ccc3cccnc3c2n1)/c1cc2ccc3cccc4ccc(c1)c2c34 ; c1cnc2c(c1)ccc1ccc(-c3cccc4c(-c5ccc6ccc7cccc8ccc5c6c78)cccc34)nc12]

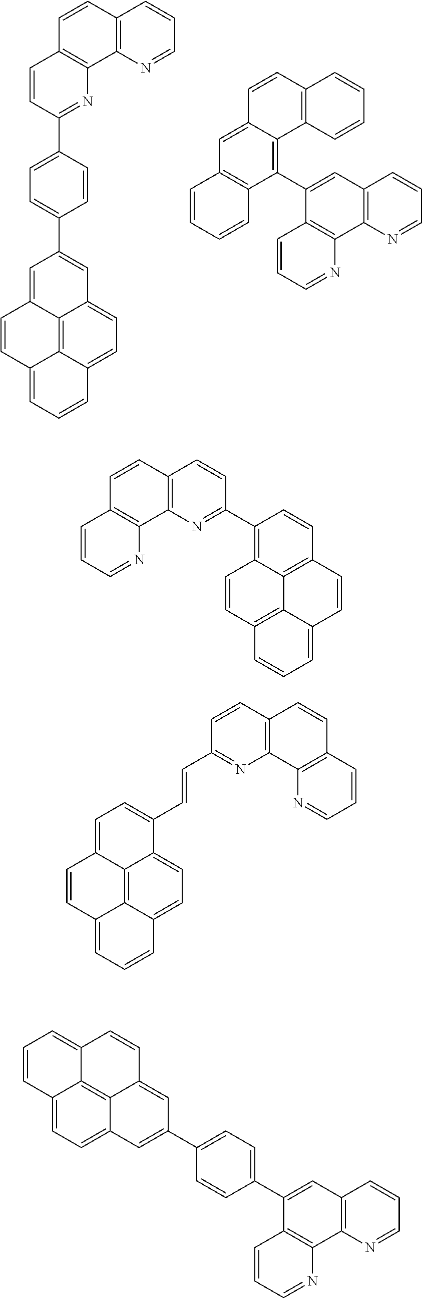

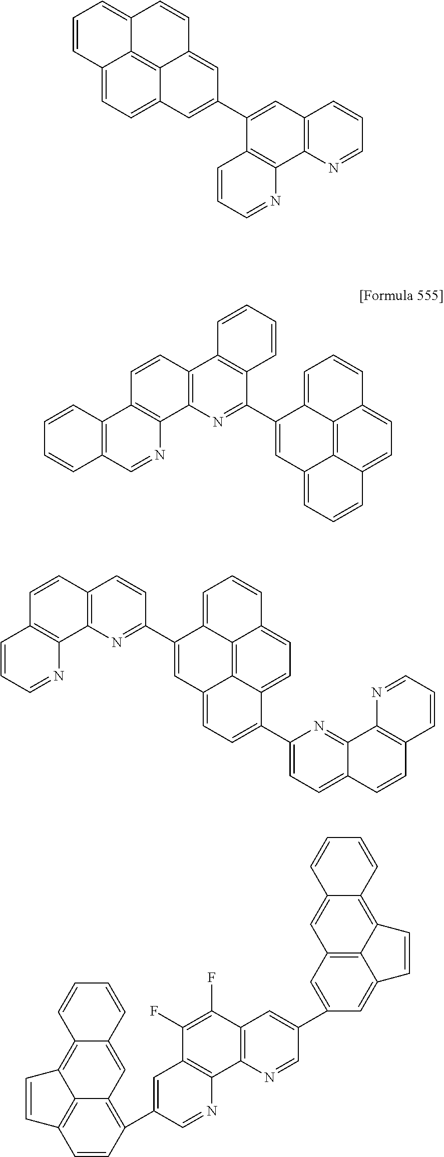

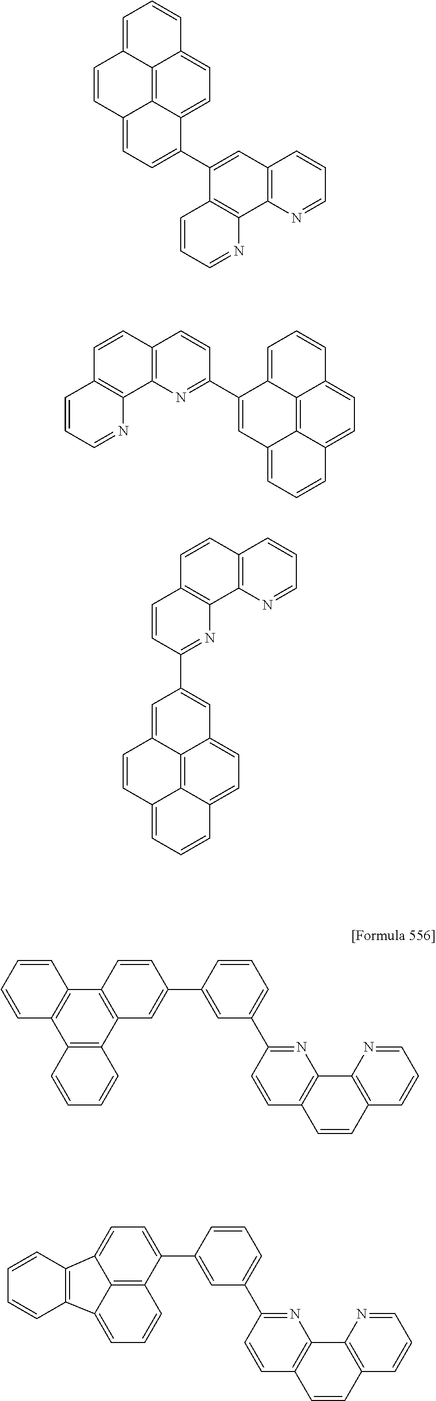 [SMILES: c1cnc2c(c1)cc(-c1ccc3ccc4cccc5ccc1c3c45)c1cccnc12 ; c1cnc2c(c1)ccc1ccc(-c3cc4cccc5ccc6cccc3c6c54)nc12 ; c1cnc2c(c1)ccc1ccc(-c3cc4ccc5cccc6ccc(c3)c4c56)nc12 ; c1cc(-c2ccc3c4ccccc4c4ccccc4c3c2)cc(-c2ccc3ccc4cccnc4c3n2)c1 ; c1cc(-c2ccc3ccc4cccnc4c3n2)cc(-c2ccc3c4c(cccc24)-c2ccccc2-3)c1]

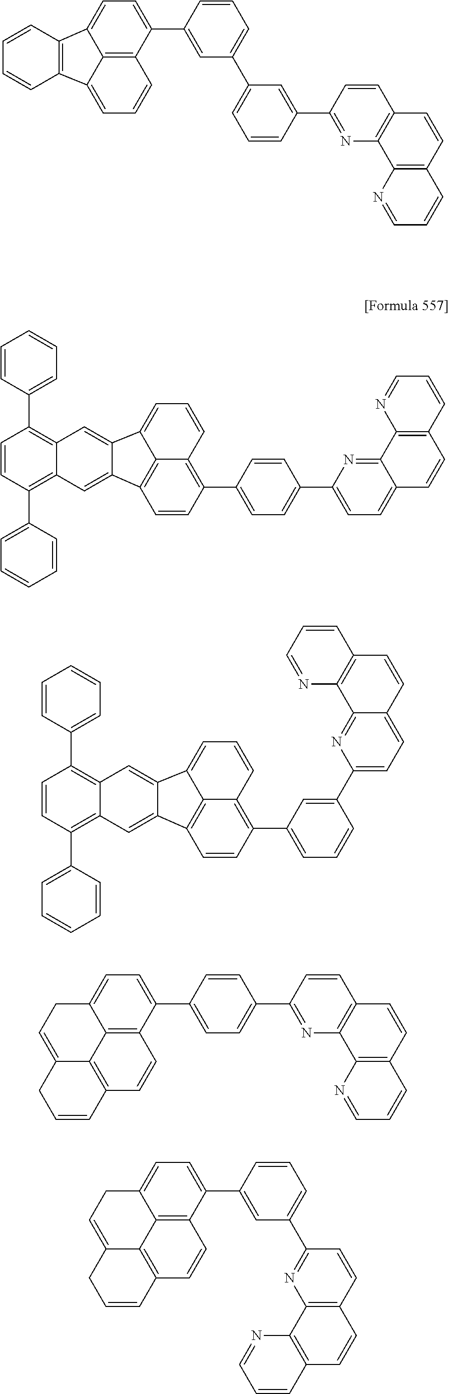 [SMILES: c1cc(-c2cccc(-c3ccc4c5c(cccc35)-c3ccccc3-4)c2)cc(-c2ccc3ccc4cccnc4c3n2)c1 ; c1ccc(-c2ccc(-c3ccccc3)c3cc4c(cc23)-c2cccc3c(-c5ccc(-c6ccc7ccc8cccnc8c7n6)cc5)ccc-4c23)cc1 ; c1ccc(-c2ccc(-c3ccccc3)c3cc4c(cc23)-c2cccc3c(-c5cccc(-c6ccc7ccc8cccnc8c7n6)c5)ccc-4c23)cc1 ; C1=Cc2ccc3c(-c4ccc(-c5ccc6ccc7cccnc7c6n5)cc4)ccc4c3c2C(=CC4)C1 ; C1=Cc2ccc3c(-c4cccc(-c5ccc6ccc7cccnc7c6n5)c4)ccc4c3c2C(=CC4)C1]

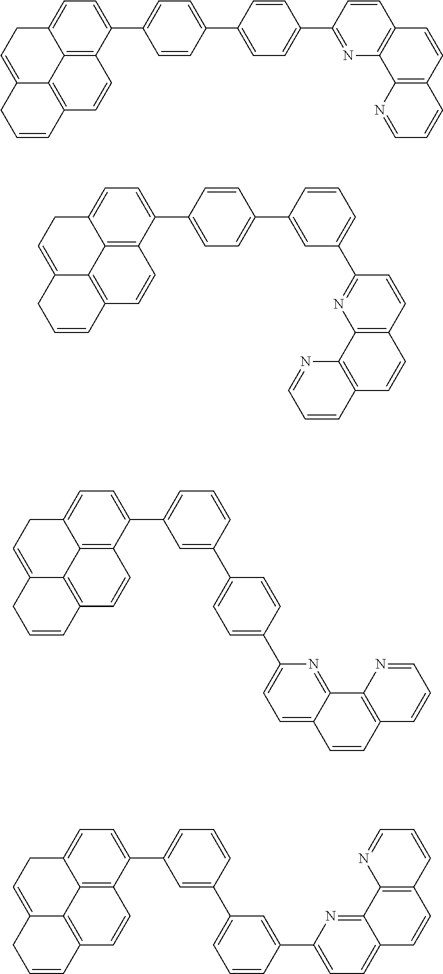 [SMILES: C1=Cc2ccc3c(-c4ccc(-c5ccc(-c6ccc7ccc8cccnc8c7n6)cc5)cc4)ccc4c3c2C(=CC4)C1 ; C1=Cc2ccc3c(-c4ccc(-c5cccc(-c6ccc7ccc8cccnc8c7n6)c5)cc4)ccc4c3c2C(=CC4)C1 ; C1=Cc2ccc3c(-c4cccc(-c5ccc(-c6ccc7ccc8cccnc8c7n6)cc5)c4)ccc4c3c2C(=CC4)C1 ; C1=Cc2ccc3c(-c4cccc(-c5cccc(-c6ccc7ccc8cccnc8c7n6)c5)c4)ccc4c3c2C(=CC4)C1]

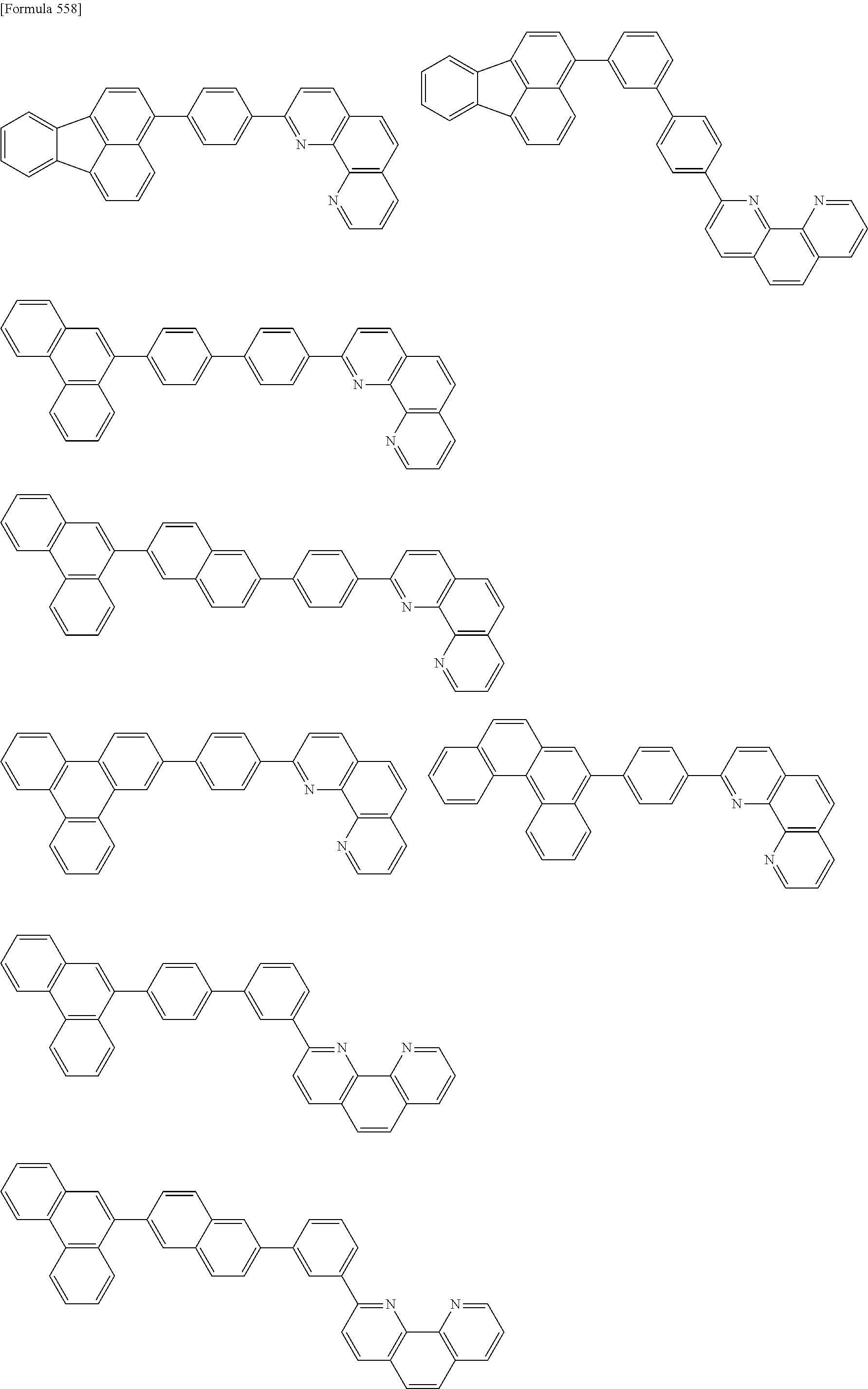

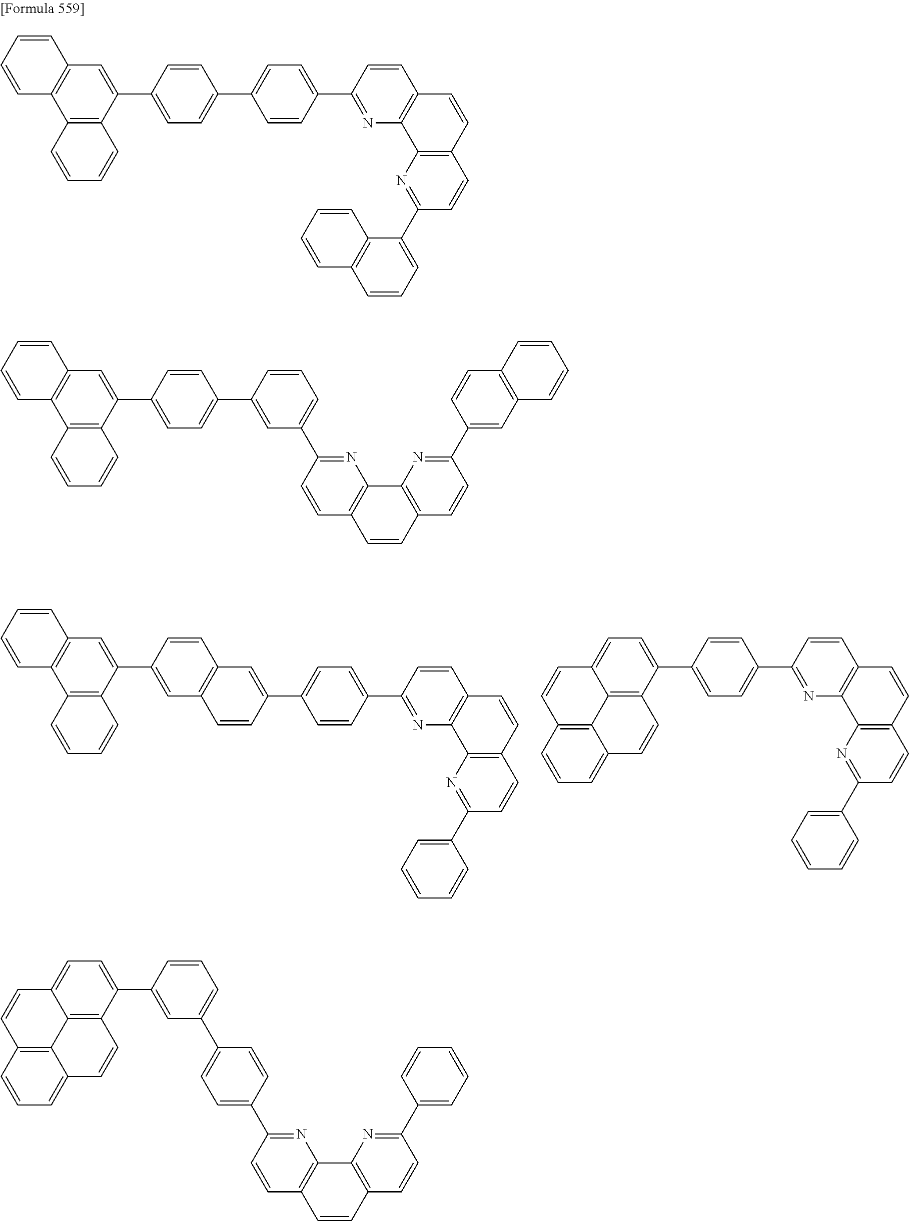

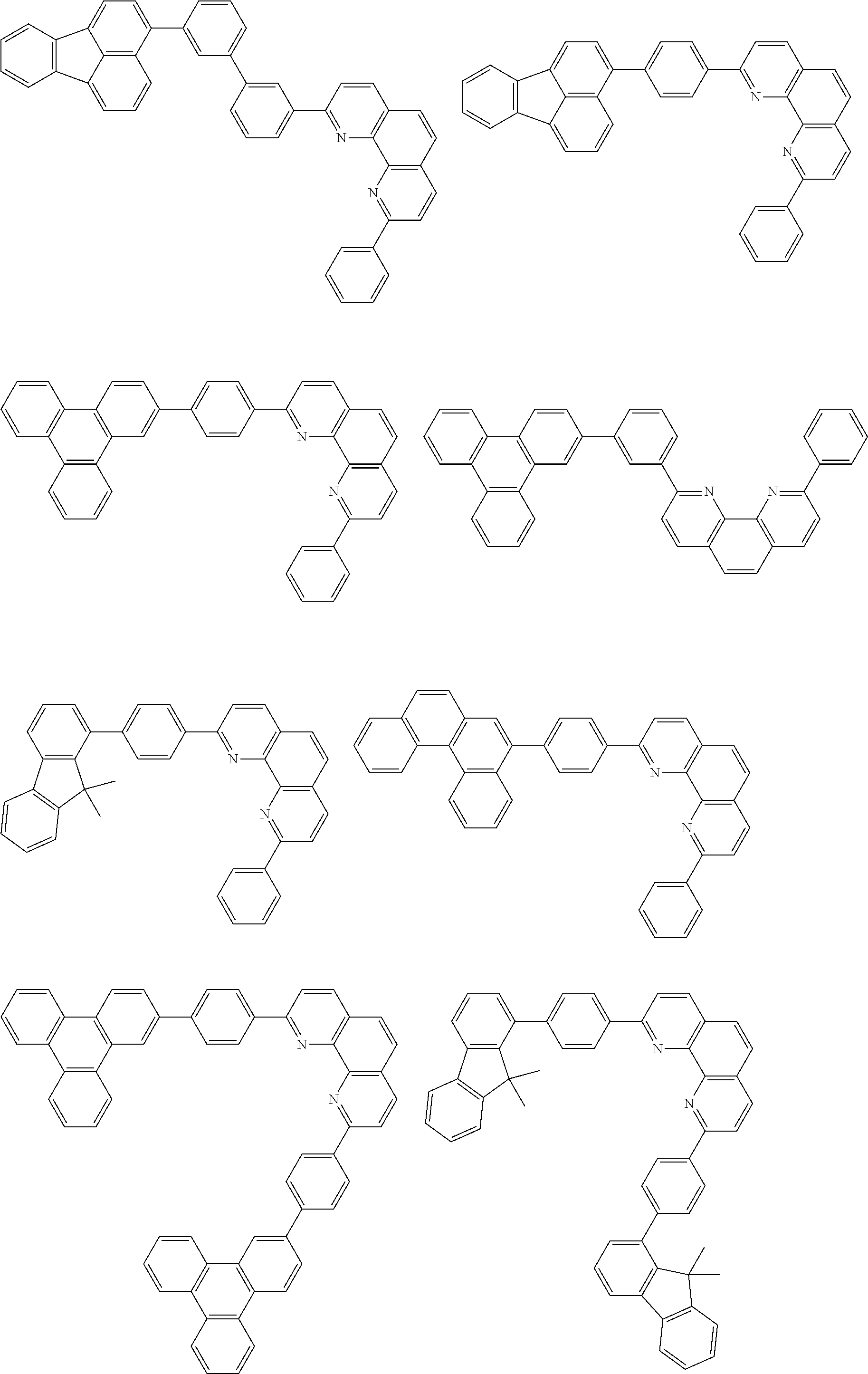

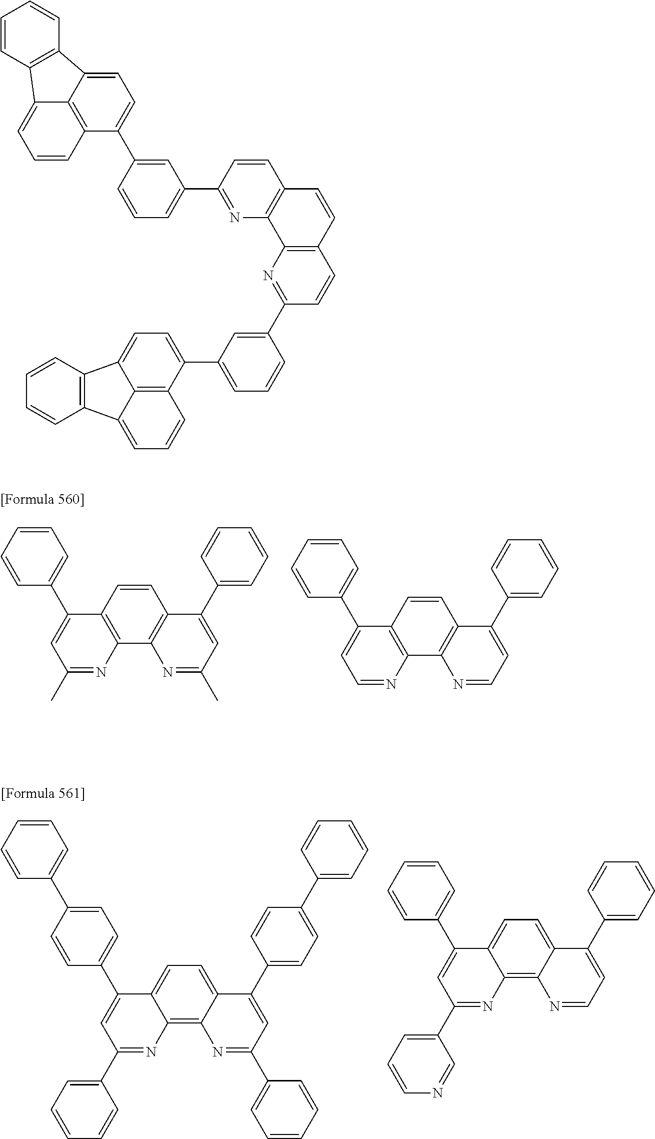

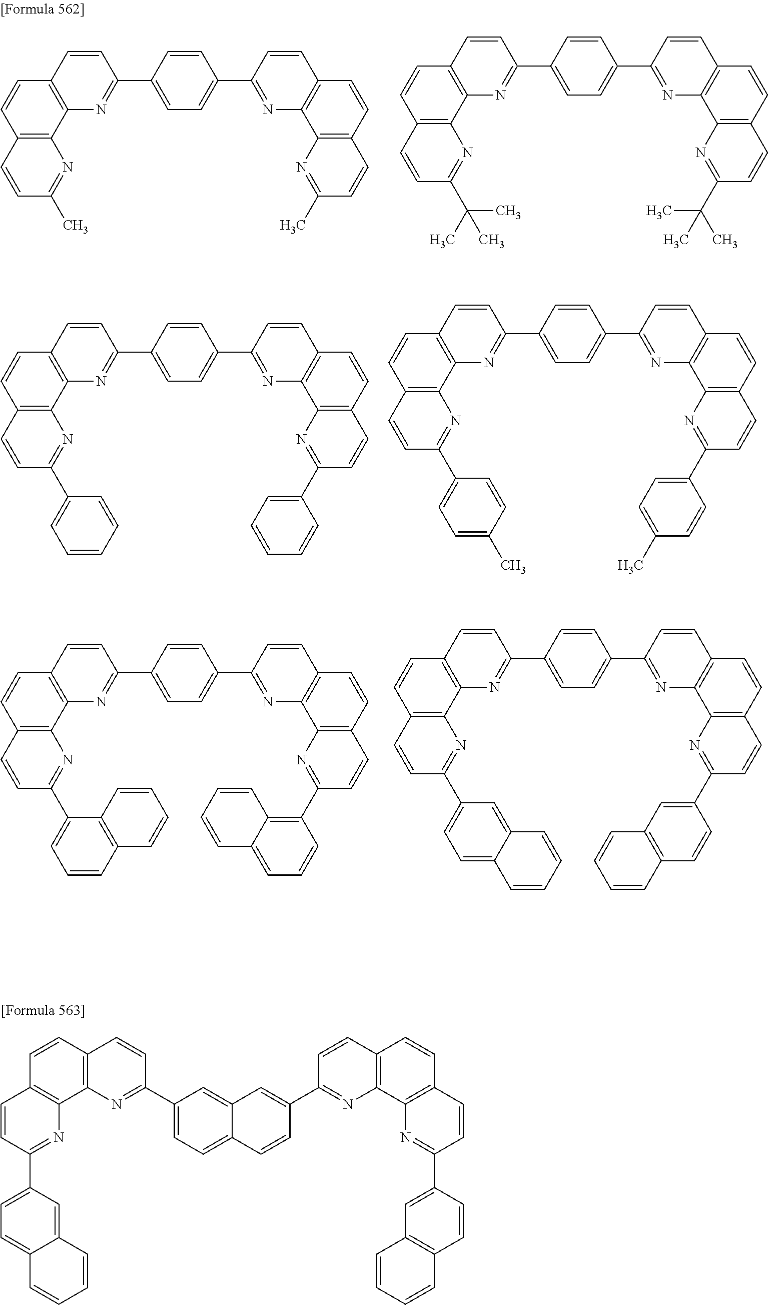

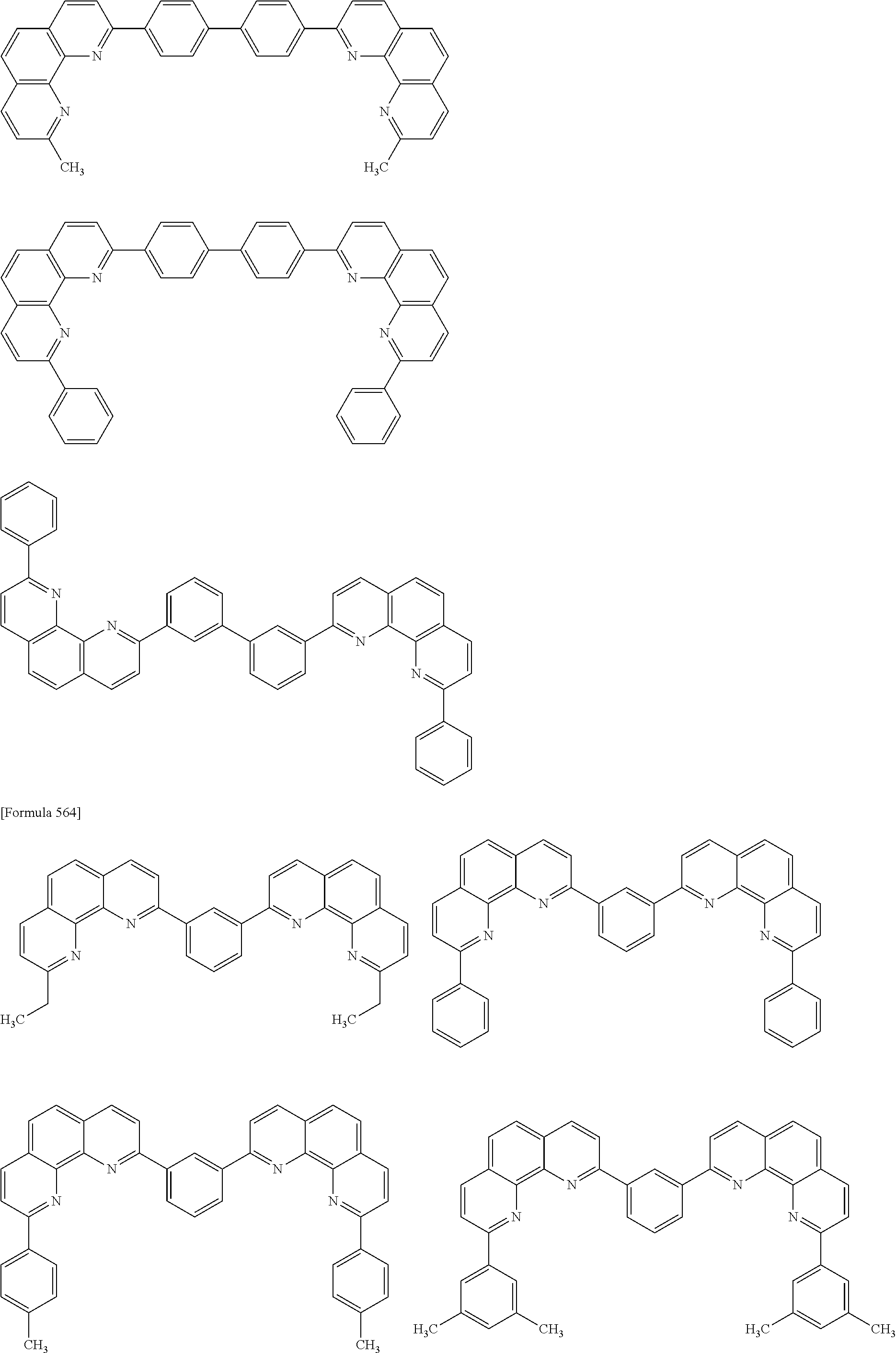

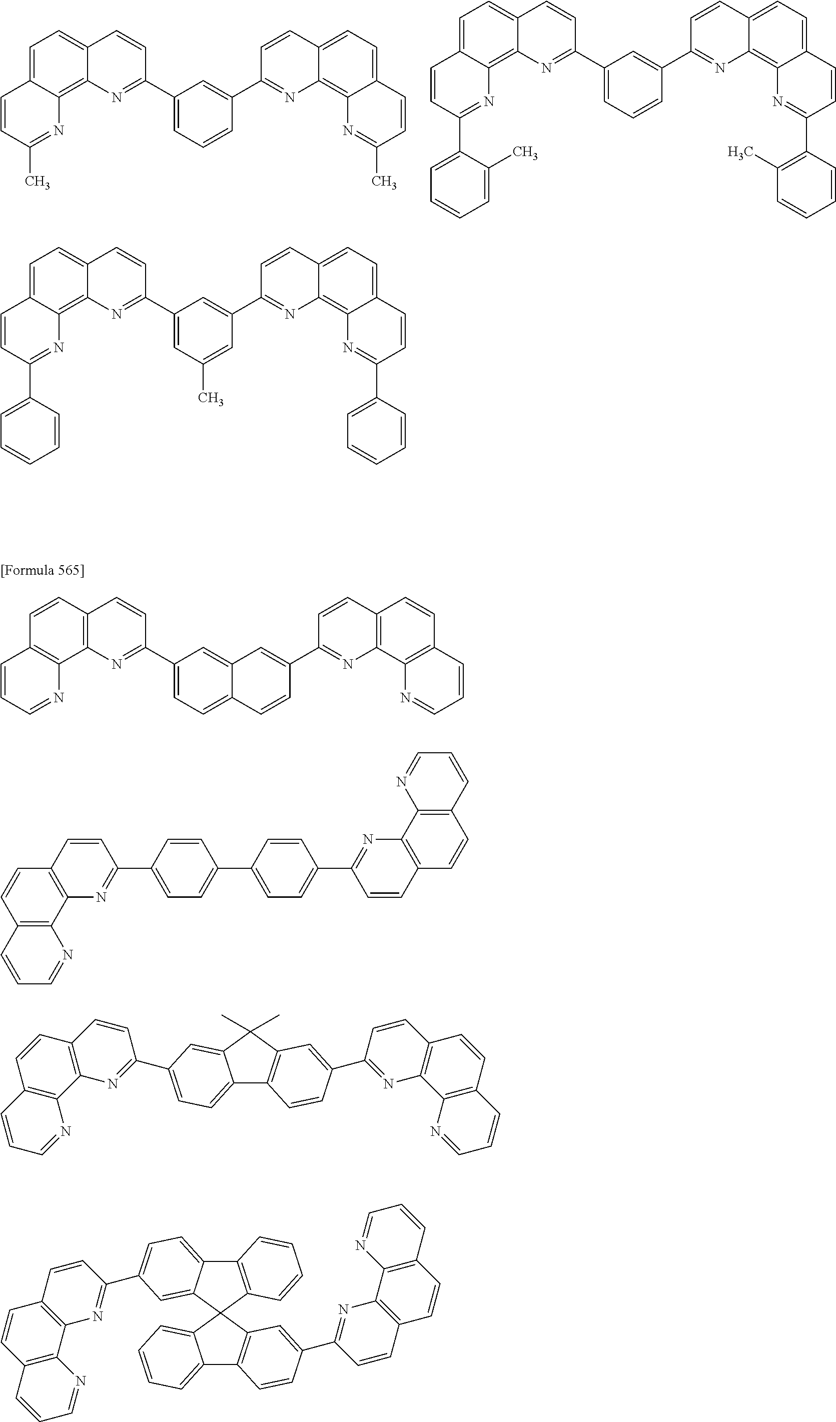

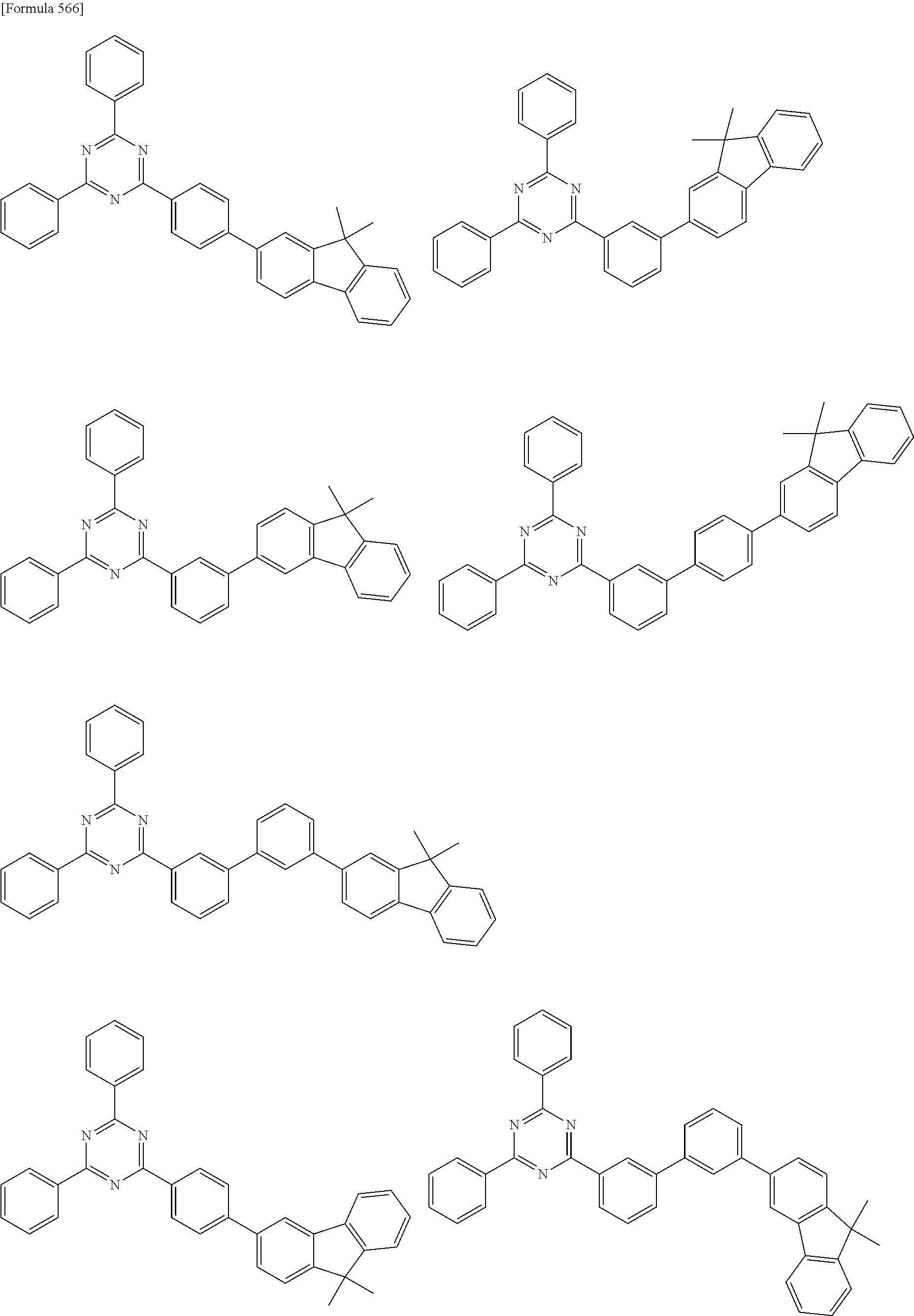

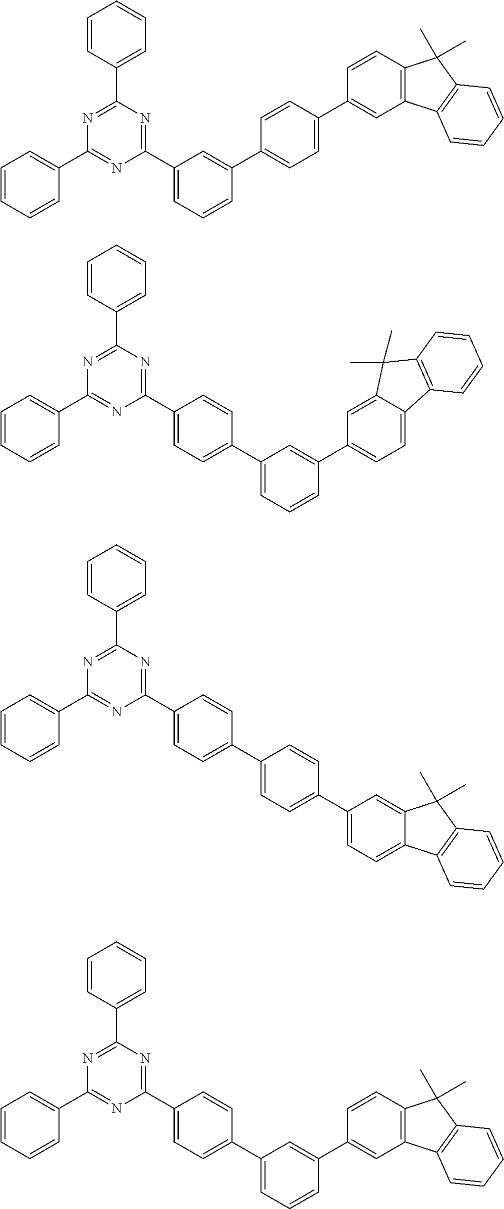 [SMILES: CC1(C)c2ccccc2-c2cc(-c3ccc(-c4cccc(-c5nc(-c6ccccc6)nc(-c6ccccc6)n5)c4)cc3)ccc21 ; CC1(C)c2ccccc2-c2ccc(-c3cccc(-c4ccc(-c5nc(-c6ccccc6)nc(-c6ccccc6)n5)cc4)c3)cc21 ; CC1(C)c2ccccc2-c2ccc(-c3ccc(-c4ccc(-c5nc(-c6ccccc6)nc(-c6ccccc6)n5)cc4)cc3)cc21 ; CC1(C)c2ccccc2-c2cc(-c3cccc(-c4ccc(-c5nc(-c6ccccc6)nc(-c6ccccc6)n5)cc4)c3)ccc21]

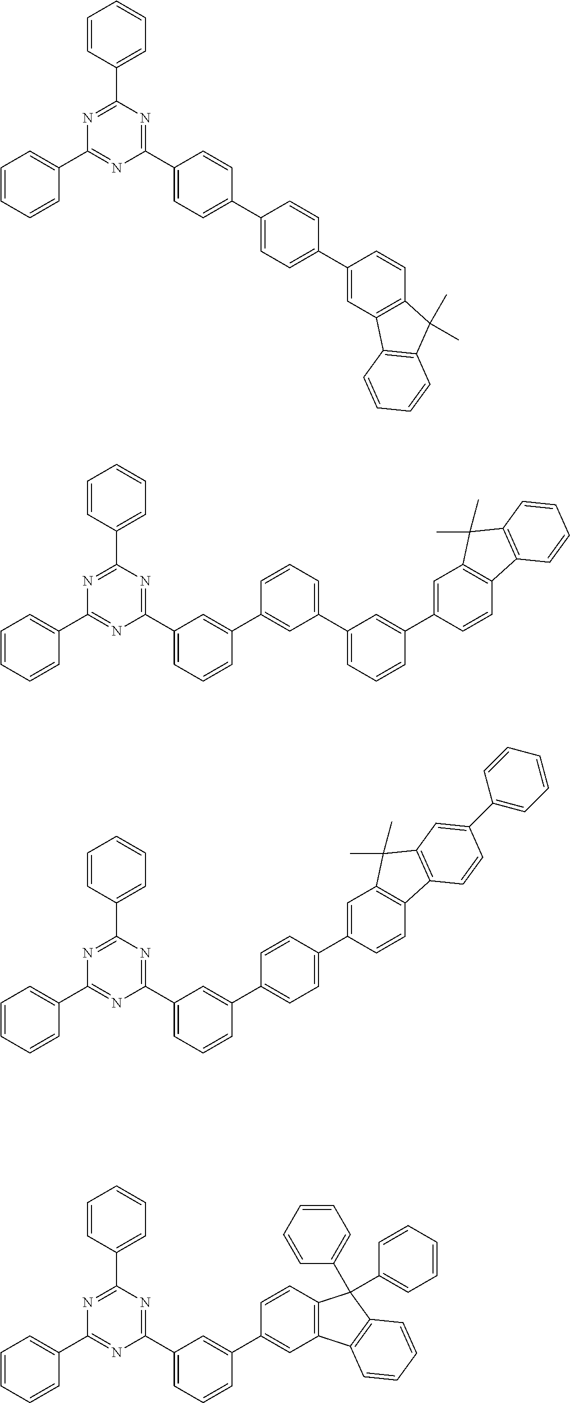 [SMILES: CC1(C)c2ccccc2-c2cc(-c3ccc(-c4ccc(-c5nc(-c6ccccc6)nc(-c6ccccc6)n5)cc4)cc3)ccc21 ; CC1(C)c2ccccc2-c2ccc(-c3cccc(-c4cccc(-c5cccc(-c6nc(-c7ccccc7)nc(-c7ccccc7)n6)c5)c4)c3)cc21 ; CC1(C)c2cc(-c3ccccc3)ccc2-c2ccc(-c3ccc(-c4cccc(-c5nc(-c6ccccc6)nc(-c6ccccc6)n5)c4)cc3)cc21 ; c1ccc(-c2nc(-c3ccccc3)nc(-c3cccc(-c4ccc5c(c4)-c4ccccc4C5(c4ccccc4)c4ccccc4)c3)n2)cc1]

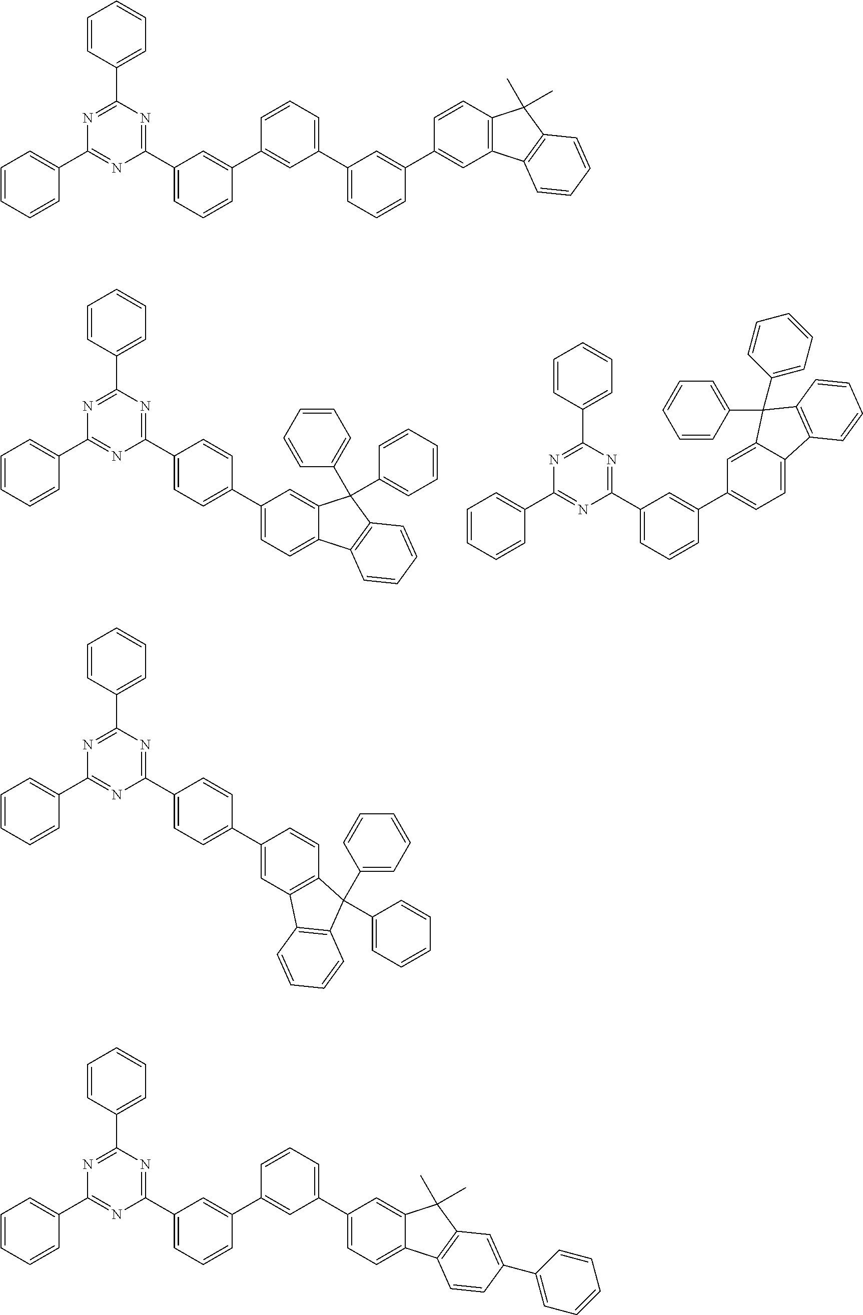

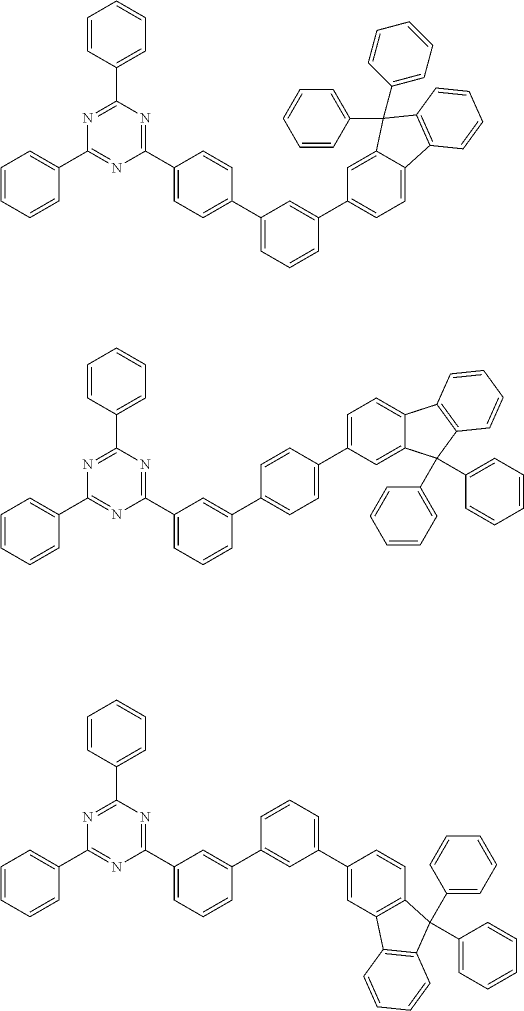 [SMILES: c1ccc(-c2nc(-c3ccccc3)nc(-c3ccc(-c4cccc(-c5ccc6c(c5)C(c5ccccc5)(c5ccccc5)c5ccccc5-6)c4)cc3)n2)cc1 ; c1ccc(-c2nc(-c3ccccc3)nc(-c3cccc(-c4ccc(-c5ccc6c(c5)C(c5ccccc5)(c5ccccc5)c5ccccc5-6)cc4)c3)n2)cc1 ; c1ccc(-c2nc(-c3ccccc3)nc(-c3cccc(-c4cccc(-c5ccc6c(c5)-c5ccccc5C6(c5ccccc5)c5ccccc5)c4)c3)n2)cc1]

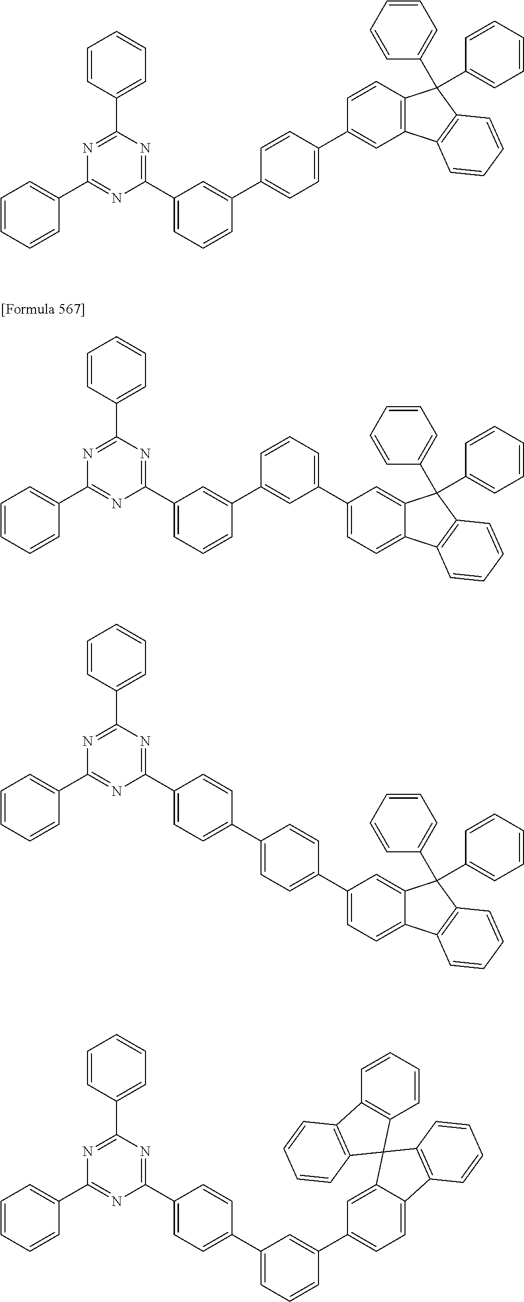 [SMILES: c1ccc(-c2nc(-c3ccccc3)nc(-c3cccc(-c4ccc(-c5ccc6c(c5)-c5ccccc5C6(c5ccccc5)c5ccccc5)cc4)c3)n2)cc1 ; c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3ccc(-c4ccc(-c5ccc6c(c5)C(c5ccccc5)(c5ccccc5)c5ccccc5-6)cc4)cc3)n2)cc1 ; c1ccc(-c2nc(-c3ccccc3)nc(-c3ccc(-c4cccc(-c5ccc6c(c5)C5(c7ccccc7-c7ccccc75)c5ccccc5-6)c4)cc3)n2)cc1]

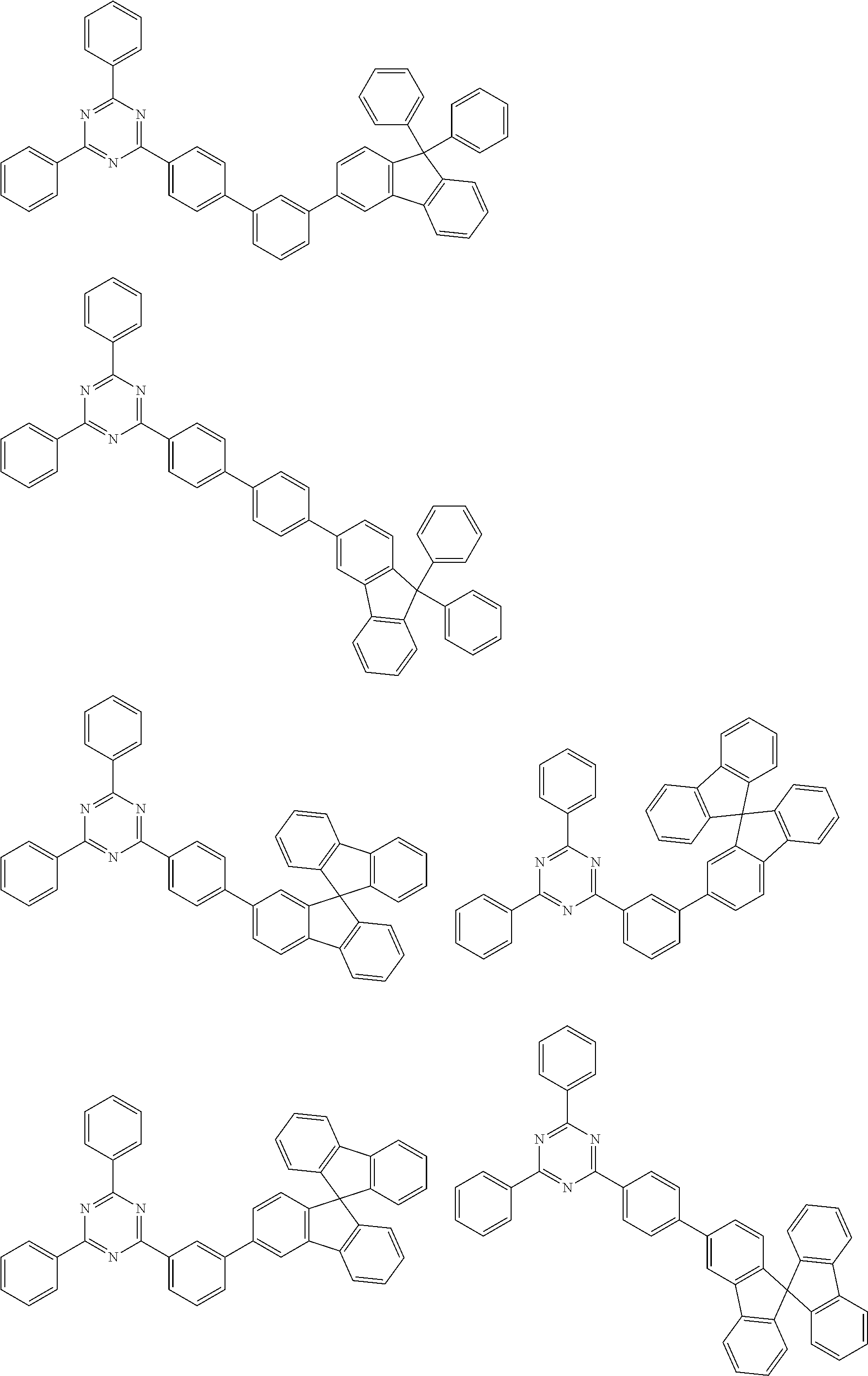

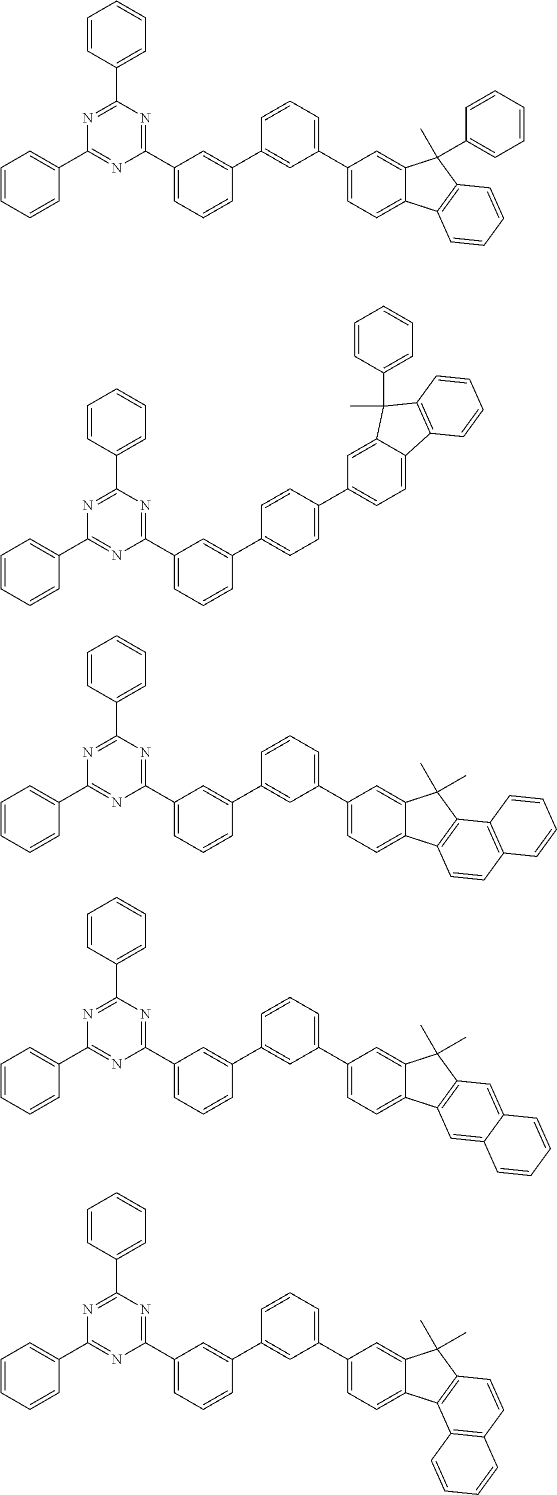 [SMILES: CC1(c2ccccc2)c2ccccc2-c2ccc(-c3cccc(-c4cccc(-c5nc(-c6ccccc6)nc(-c6ccccc6)n5)c4)c3)cc21 ; CC1(c2ccccc2)c2ccccc2-c2ccc(-c3ccc(-c4cccc(-c5nc(-c6ccccc6)nc(-c6ccccc6)n5)c4)cc3)cc21 ; CC1(C)c2cc(-c3cccc(-c4cccc(-c5nc(-c6ccccc6)nc(-c6ccccc6)n5)c4)c3)ccc2-c2ccc3ccccc3c21 ; CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21]

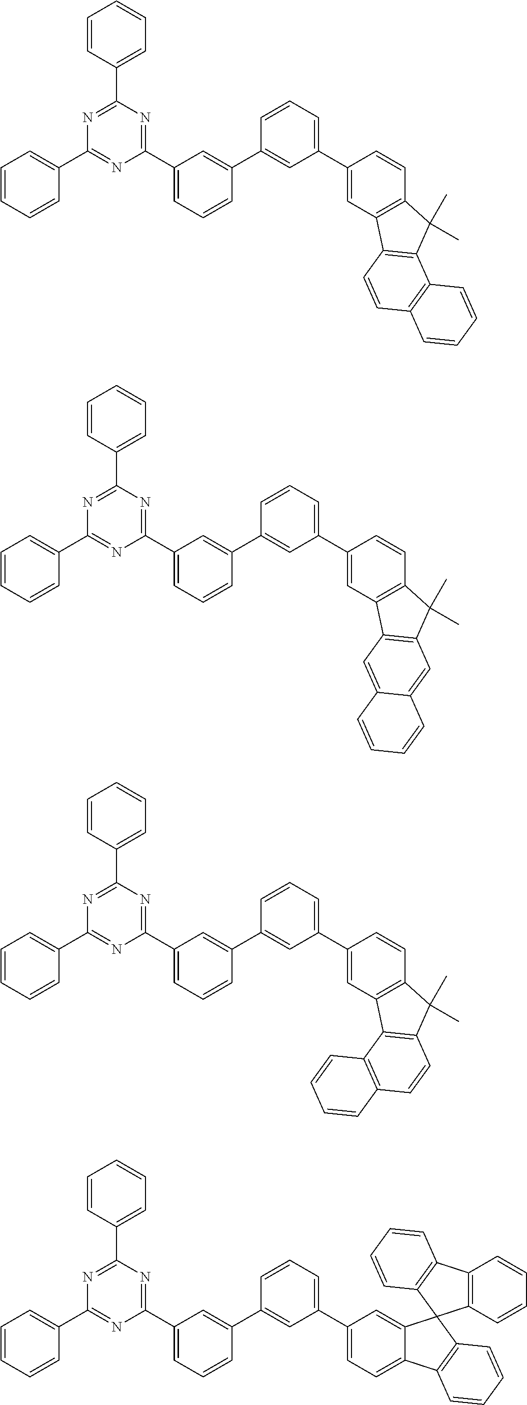 [SMILES: CC1(C)c2ccc(-c3cccc(-c4cccc(-c5nc(-c6ccccc6)nc(-c6ccccc6)n5)c4)c3)cc2-c2ccc3ccccc3c21 ; CC1(C)c2ccc(-c3cccc(-c4cccc(-c5nc(-c6ccccc6)nc(-c6ccccc6)n5)c4)c3)cc2-c2cc3ccccc3cc21 ; CC1(C)c2ccc(-c3cccc(-c4cccc(-c5nc(-c6ccccc6)nc(-c6ccccc6)n5)c4)c3)cc2-c2c1ccc1ccccc21 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

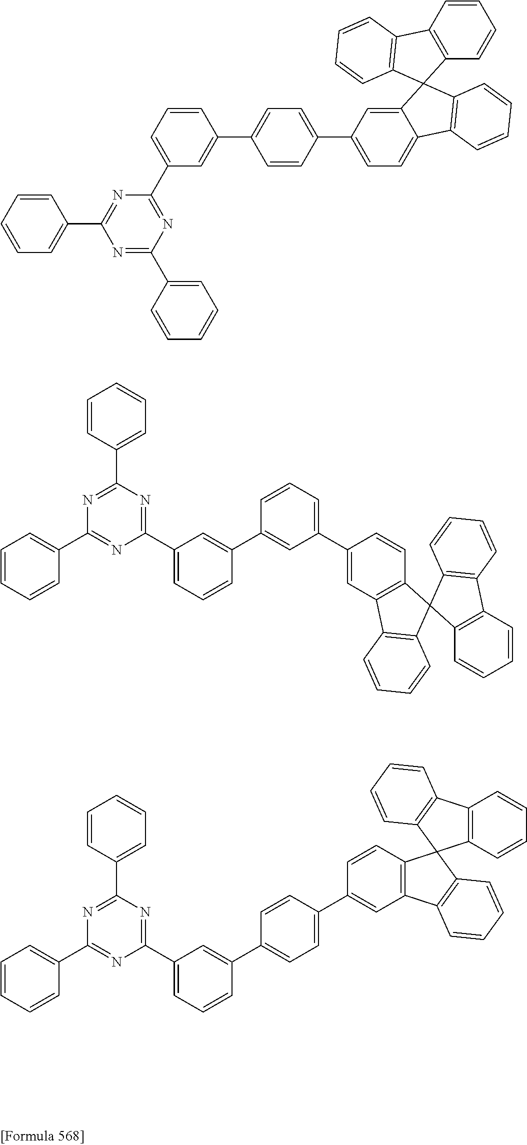 [SMILES: c1ccc(-c2nc(-c3ccccc3)nc(-c3cccc(-c4ccc(-c5ccc6c(c5)C5(c7ccccc7-c7ccccc75)c5ccccc5-6)cc4)c3)n2)cc1 ; c1ccc(-c2nc(-c3ccccc3)nc(-c3cccc(-c4cccc(-c5ccc6c(c5)-c5ccccc5C65c6ccccc6-c6ccccc65)c4)c3)n2)cc1 ; c1ccc(-c2nc(-c3ccccc3)nc(-c3cccc(-c4ccc(-c5ccc6c(c5)-c5ccccc5C65c6ccccc6-c6ccccc65)cc4)c3)n2)cc1]

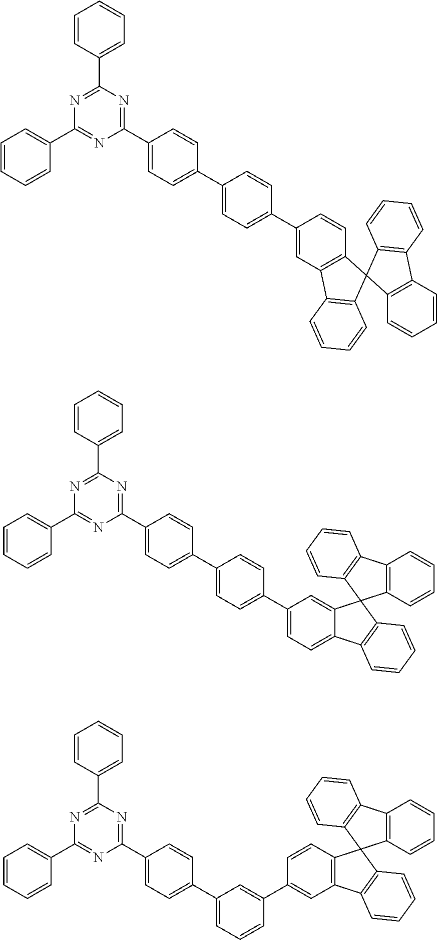 [SMILES: c1ccc(-c2nc(-c3ccccc3)nc(-c3ccc(-c4ccc(-c5ccc6c(c5)-c5ccccc5C65c6ccccc6-c6ccccc65)cc4)cc3)n2)cc1 ; c1ccc(-c2nc(-c3ccccc3)nc(-c3ccc(-c4ccc(-c5ccc6c(c5)C5(c7ccccc7-c7ccccc75)c5ccccc5-6)cc4)cc3)n2)cc1 ; c1ccc(-c2nc(-c3ccccc3)nc(-c3ccc(-c4cccc(-c5ccc6c(c5)-c5ccccc5C65c6ccccc6-c6ccccc65)c4)cc3)n2)cc1]

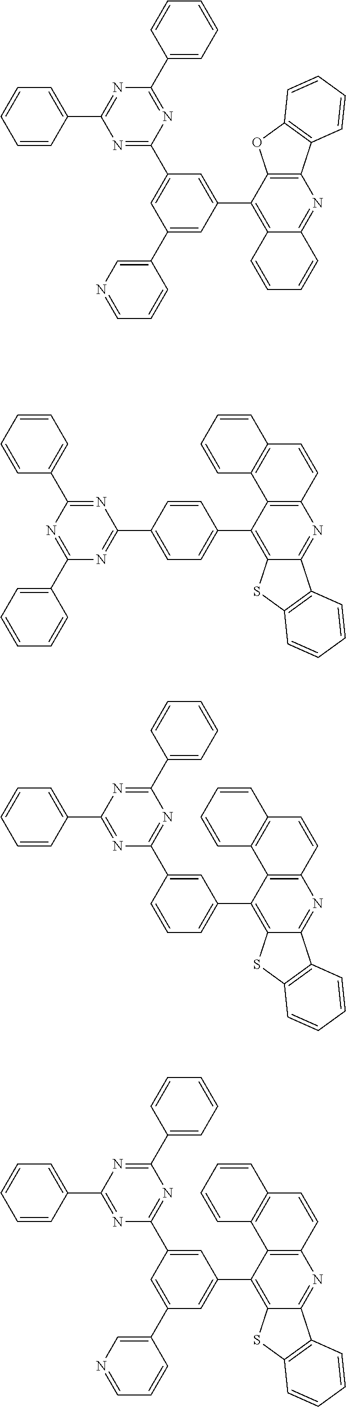 [SMILES: c1ccc(-c2nc(-c3ccccc3)nc(-c3cc(-c4cccnc4)cc(-c4c5ccccc5nc5c4oc4ccccc45)c3)n2)cc1 ; c1ccc(-c2nc(-c3ccccc3)nc(-c3ccc(-c4c5sc6ccccc6c5nc5ccc6ccccc6c45)cc3)n2)cc1 ; c1ccc(-c2nc(-c3ccccc3)nc(-c3cccc(-c4c5sc6ccccc6c5nc5ccc6ccccc6c45)c3)n2)cc1 ; c1ccc(-c2nc(-c3ccccc3)nc(-c3cc(-c4cccnc4)cc(-c4c5sc6ccccc6c5nc5ccc6ccccc6c45)c3)n2)cc1]

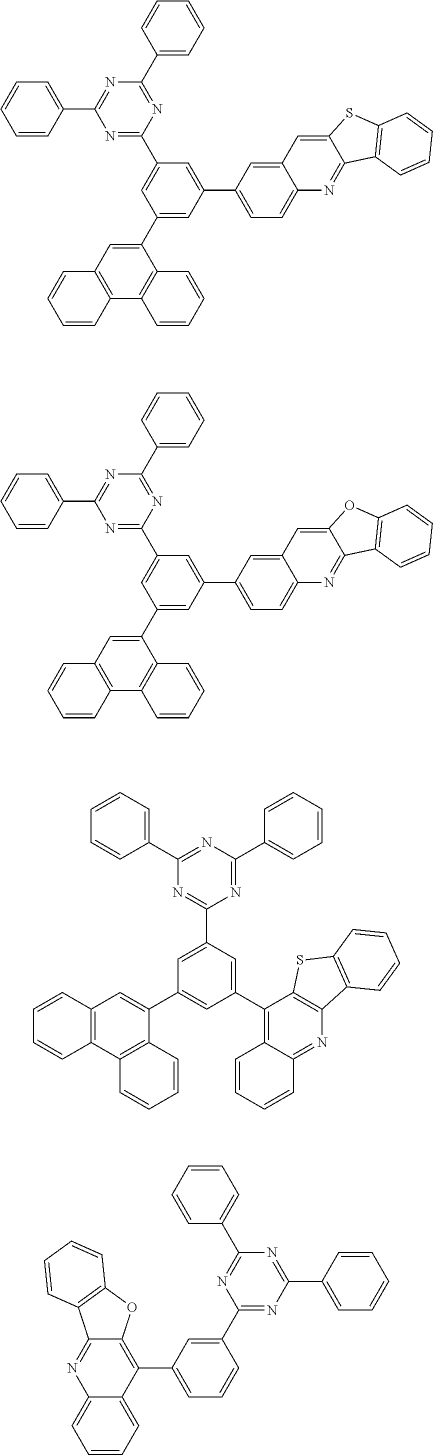 [SMILES: c1ccc(-c2nc(-c3ccccc3)nc(-c3cc(-c4ccc5nc6c(cc5c4)sc4ccccc46)cc(-c4cc5ccccc5c5ccccc45)c3)n2)cc1 ; c1ccc(-c2nc(-c3ccccc3)nc(-c3cc(-c4ccc5nc6c(cc5c4)oc4ccccc46)cc(-c4cc5ccccc5c5ccccc45)c3)n2)cc1 ; c1ccc(-c2nc(-c3ccccc3)nc(-c3cc(-c4cc5ccccc5c5ccccc45)cc(-c4c5ccccc5nc5c4sc4ccccc45)c3)n2)cc1 ; c1ccc(-c2nc(-c3ccccc3)nc(-c3cccc(-c4c5ccccc5nc5c4oc4ccccc45)c3)n2)cc1]

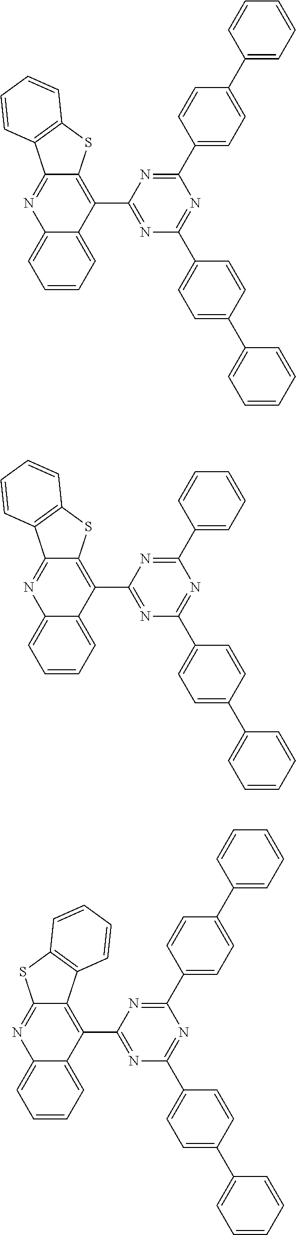

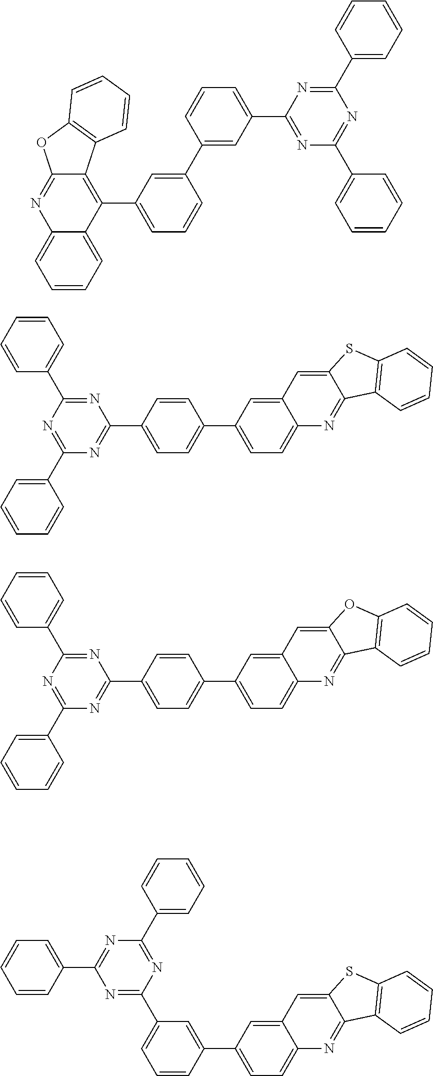

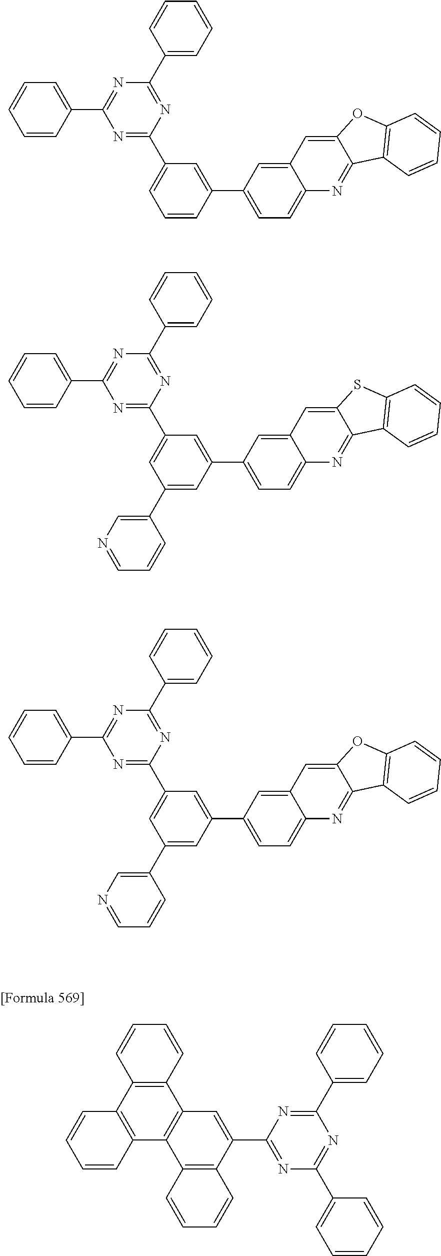 [SMILES: c1ccc(-c2nc(-c3ccccc3)nc(-c3cccc(-c4ccc5nc6c(cc5c4)oc4ccccc46)c3)n2)cc1 ; c1ccc(-c2nc(-c3ccccc3)nc(-c3cc(-c4cccnc4)cc(-c4ccc5nc6c(cc5c4)sc4ccccc46)c3)n2)cc1 ; c1ccc(-c2nc(-c3ccccc3)nc(-c3cc(-c4cccnc4)cc(-c4ccc5nc6c(cc5c4)oc4ccccc46)c3)n2)cc1 ; c1ccc(-c2nc(-c3ccccc3)nc(-c3cc4c5ccccc5c5ccccc5c4c4ccccc34)n2)cc1]

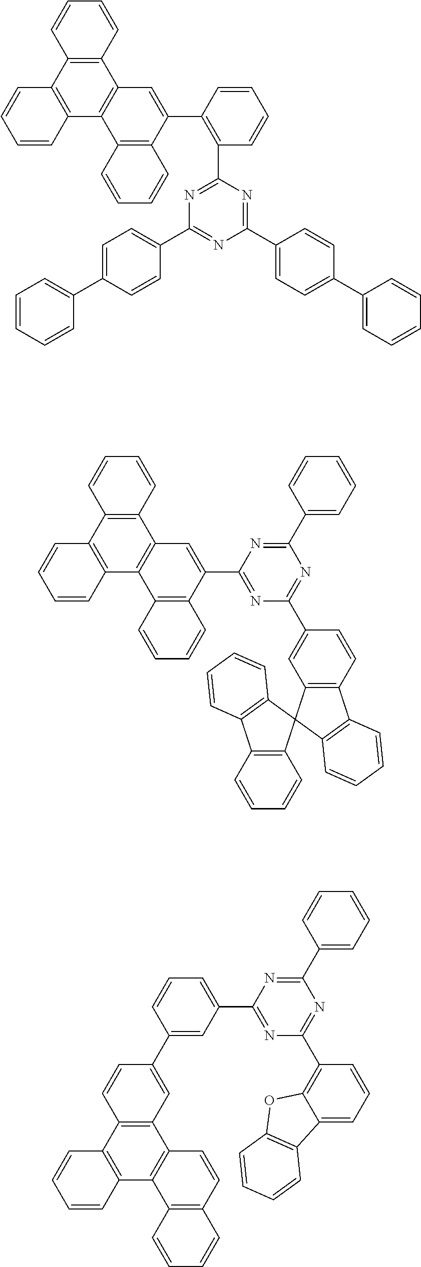 [SMILES: c1ccc(-c2ccc(-c3nc(-c4ccc(-c5ccccc5)cc4)nc(-c4ccccc4-c4cc5c6ccccc6c6ccccc6c5c5ccccc45)n3)cc2)cc1 ; c1ccc(-c2nc(-c3ccc4c(c3)C3(c5ccccc5-c5ccccc53)c3ccccc3-4)nc(-c3cc4c5ccccc5c5ccccc5c4c4ccccc34)n2)cc1 ; c1ccc(-c2nc(-c3cccc(-c4ccc5c6ccccc6c6c7ccccc7ccc6c5c4)c3)nc(-c3cccc4c3oc3ccccc34)n2)cc1]

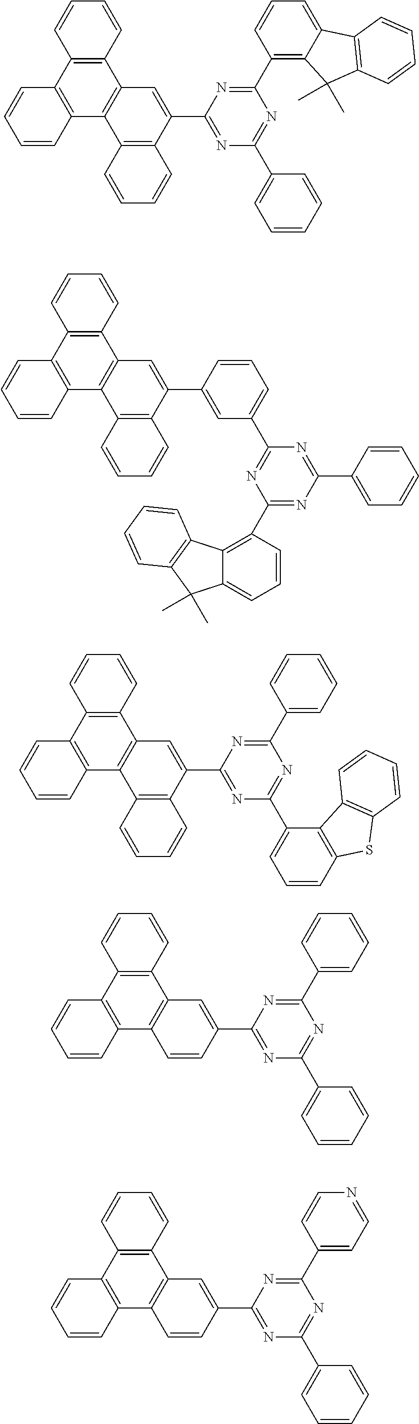

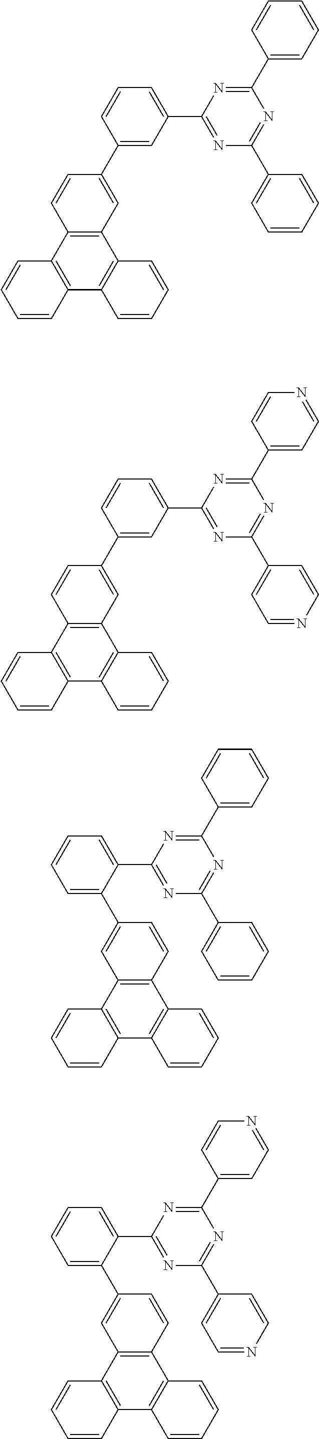 [SMILES: c1ccc(-c2nc(-c3ccccc3)nc(-c3cccc(-c4ccc5c6ccccc6c6ccccc6c5c4)c3)n2)cc1 ; c1cc(-c2ccc3c4ccccc4c4ccccc4c3c2)cc(-c2nc(-c3ccncc3)nc(-c3ccncc3)n2)c1 ; c1ccc(-c2nc(-c3ccccc3)nc(-c3ccccc3-c3ccc4c5ccccc5c5ccccc5c4c3)n2)cc1 ; c1ccc(-c2nc(-c3ccncc3)nc(-c3ccncc3)n2)c(-c2ccc3c4ccccc4c4ccccc4c3c2)c1]

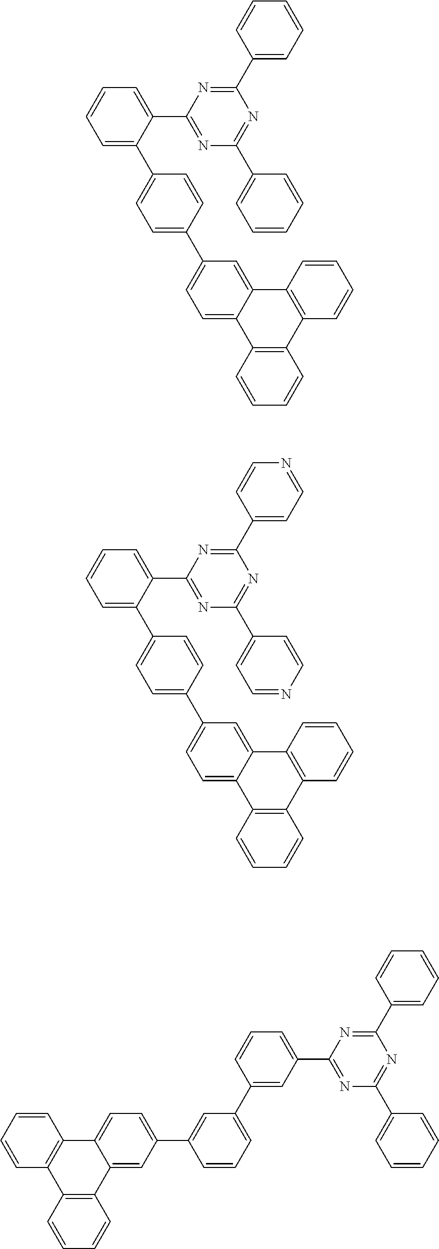 [SMILES: c1ccc(-c2nc(-c3ccccc3)nc(-c3ccccc3-c3ccc(-c4ccc5c6ccccc6c6ccccc6c5c4)cc3)n2)cc1 ; c1ccc(-c2nc(-c3ccncc3)nc(-c3ccncc3)n2)c(-c2ccc(-c3ccc4c5ccccc5c5ccccc5c4c3)cc2)c1 ; c1ccc(-c2nc(-c3ccccc3)nc(-c3cccc(-c4cccc(-c5ccc6c7ccccc7c7ccccc7c6c5)c4)c3)n2)cc1]

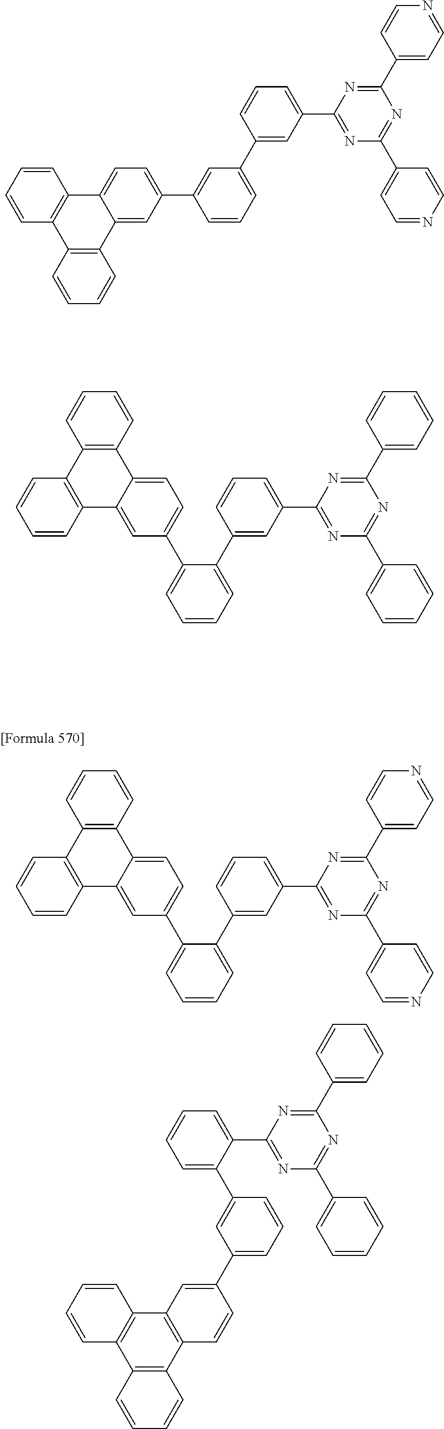

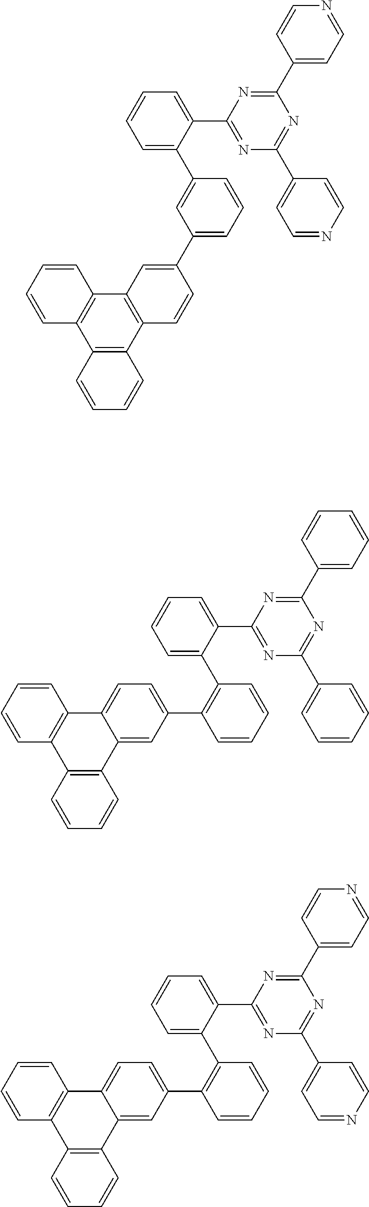 [SMILES: c1cc(-c2ccc3c4ccccc4c4ccccc4c3c2)cc(-c2ccccc2-c2nc(-c3ccncc3)nc(-c3ccncc3)n2)c1 ; c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2nc(-c3ccncc3)nc(-c3ccncc3)n2)c(-c2ccc3c4ccccc4c4ccccc4c3c2)c1]

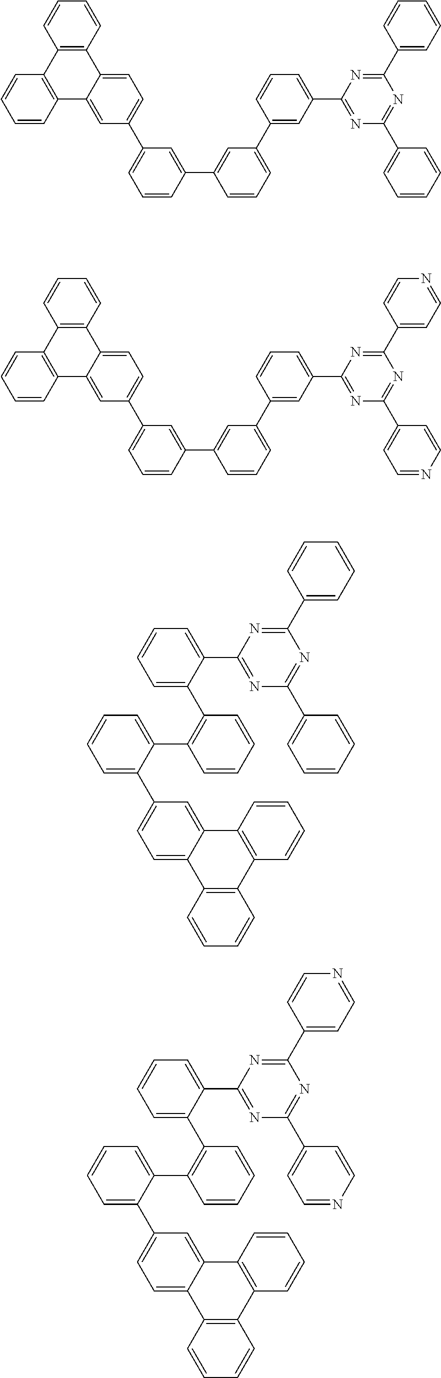 [SMILES: c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1cc(-c2cccc(-c3ccc4c5ccccc5c5ccccc5c4c3)c2)cc(-c2cccc(-c3nc(-c4ccncc4)nc(-c4ccncc4)n3)c2)c1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2nc(-c3ccncc3)nc(-c3ccncc3)n2)c(-c2ccc3c4ccccc4c4ccccc4c3c2)c1]

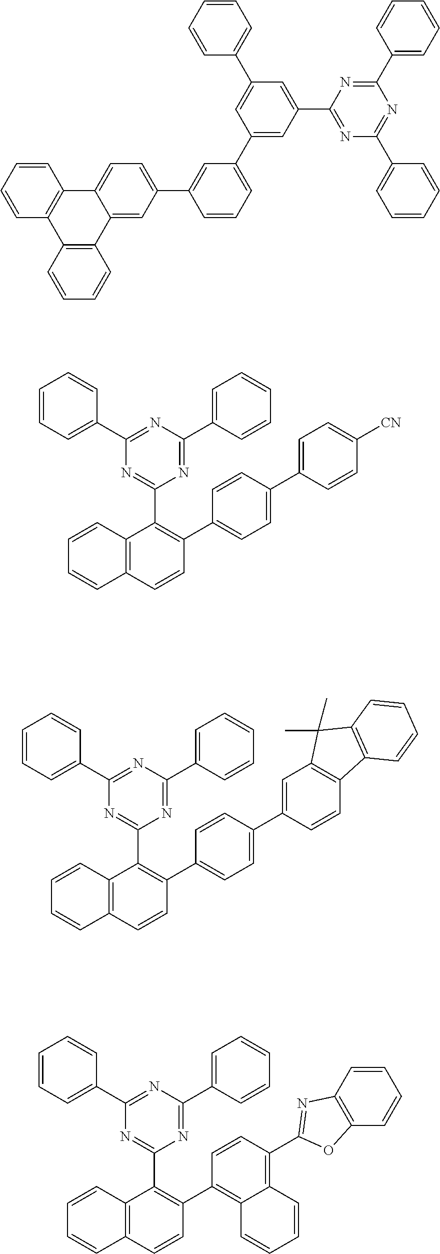 [SMILES: c1ccc(-c2cc(-c3cccc(-c4ccc5c6ccccc6c6ccccc6c5c4)c3)cc(-c3nc(-c4ccccc4)nc(-c4ccccc4)n3)c2)cc1 ; N#Cc1ccc(-c2ccc(-c3ccc4ccccc4c3-c3nc(-c4ccccc4)nc(-c4ccccc4)n3)cc2)cc1 ; CC1(C)c2ccccc2-c2ccc(-c3ccc(-c4ccc5ccccc5c4-c4nc(-c5ccccc5)nc(-c5ccccc5)n4)cc3)cc21 ; c1ccc(-c2nc(-c3ccccc3)nc(-c3c(-c4ccc(-c5nc6ccccc6o5)c5ccccc45)ccc4ccccc34)n2)cc1]

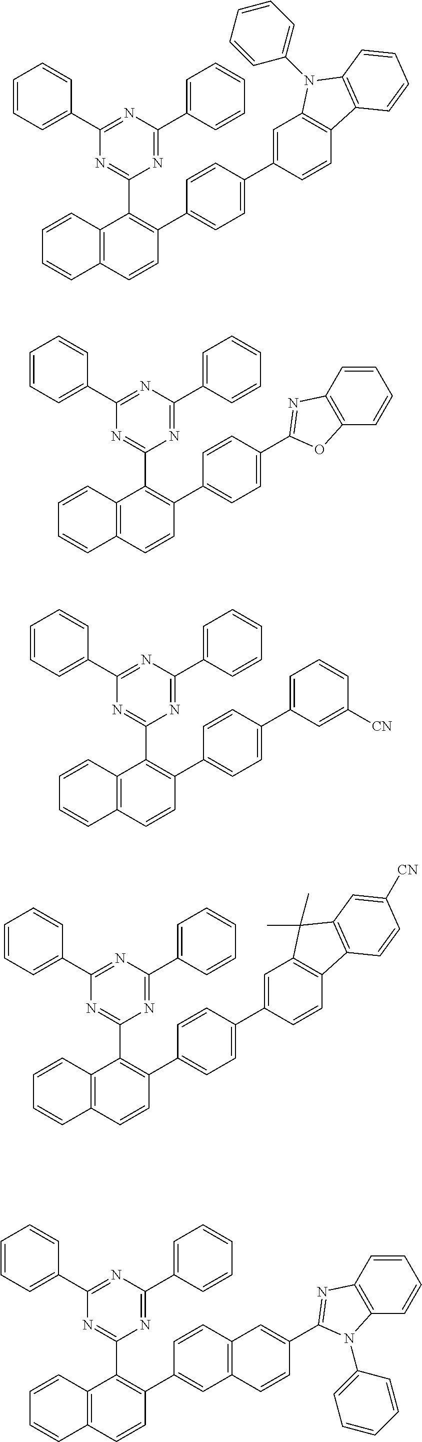 [SMILES: c1ccc(-c2nc(-c3ccccc3)nc(-c3c(-c4ccc(-c5ccc6c7ccccc7n(-c7ccccc7)c6c5)cc4)ccc4ccccc34)n2)cc1 ; c1ccc(-c2nc(-c3ccccc3)nc(-c3c(-c4ccc(-c5nc6ccccc6o5)cc4)ccc4ccccc34)n2)cc1 ; N#Cc1cccc(-c2ccc(-c3ccc4ccccc4c3-c3nc(-c4ccccc4)nc(-c4ccccc4)n3)cc2)c1 ; CC1(C)c2cc(C#N)ccc2-c2ccc(-c3ccc(-c4ccc5ccccc5c4-c4nc(-c5ccccc5)nc(-c5ccccc5)n4)cc3)cc21 ; c1ccc(-c2nc(-c3ccccc3)nc(-c3c(-c4ccc5cc(-c6nc7ccccc7n6-c6ccccc6)ccc5c4)ccc4ccccc34)n2)cc1]

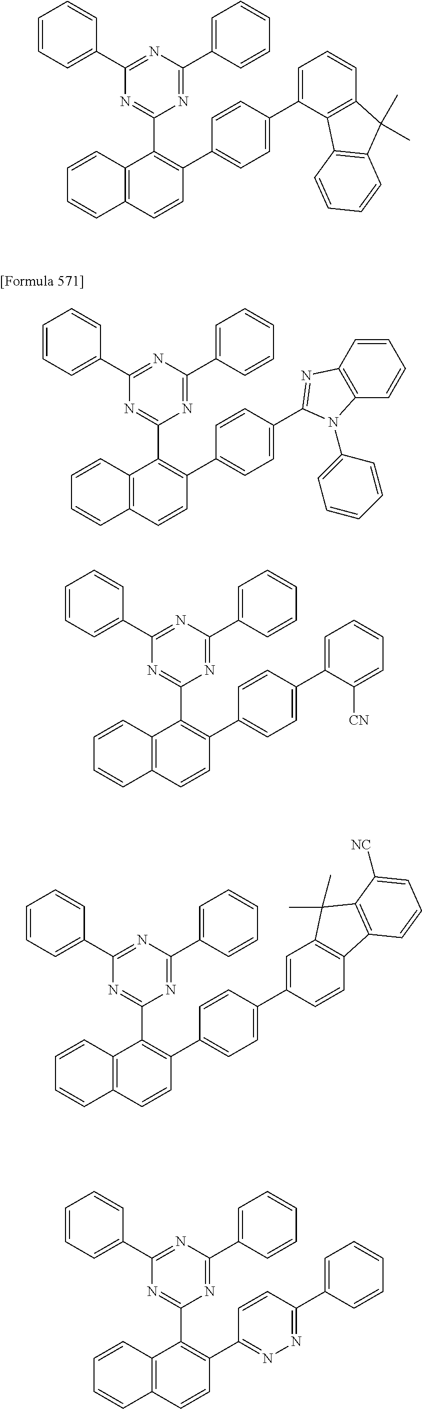

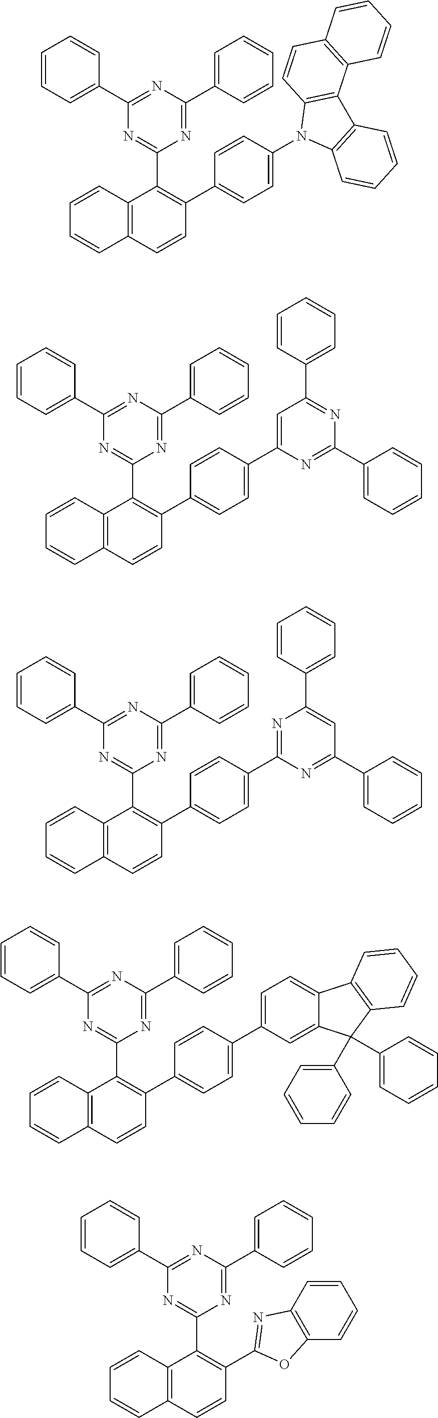 [SMILES: c1ccc(-c2nc(-c3ccccc3)nc(-c3c(-c4ccc(-n5c6ccccc6c6c7ccccc7ccc65)cc4)ccc4ccccc34)n2)cc1 ; c1ccc(-c2cc(-c3ccc(-c4ccc5ccccc5c4-c4nc(-c5ccccc5)nc(-c5ccccc5)n4)cc3)nc(-c3ccccc3)n2)cc1 ; c1ccc(-c2cc(-c3ccccc3)nc(-c3ccc(-c4ccc5ccccc5c4-c4nc(-c5ccccc5)nc(-c5ccccc5)n4)cc3)n2)cc1 ; c1ccc(-c2nc(-c3ccccc3)nc(-c3c(-c4ccc(-c5ccc6c(c5)C(c5ccccc5)(c5ccccc5)c5ccccc5-6)cc4)ccc4ccccc34)n2)cc1 ; c1ccc(-c2nc(-c3ccccc3)nc(-c3c(-c4nc5ccccc5o4)ccc4ccccc34)n2)cc1]

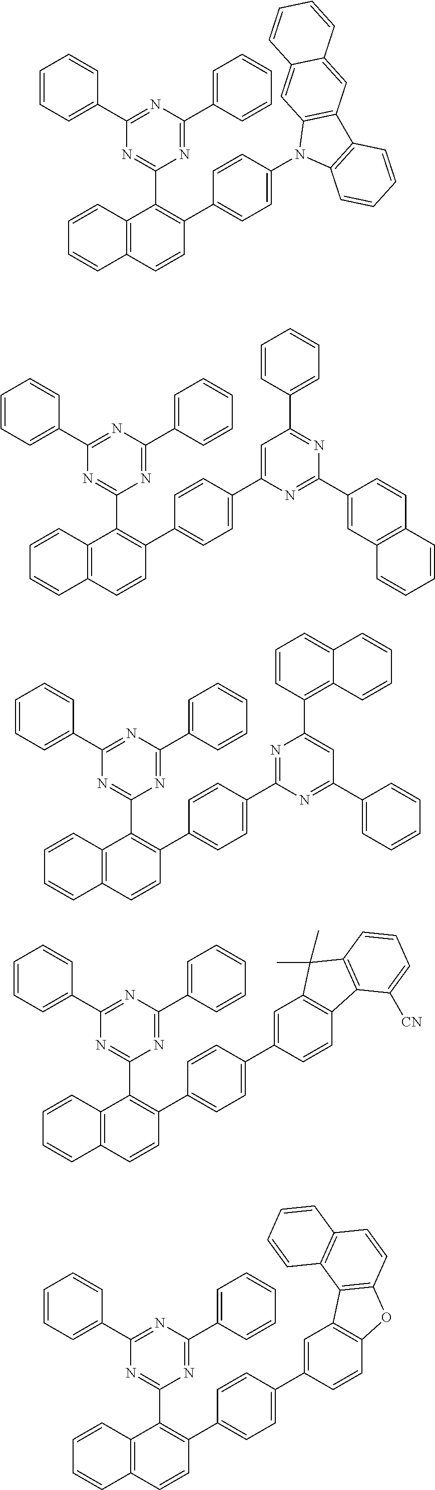 [SMILES: c1ccc(-c2nc(-c3ccccc3)nc(-c3c(-c4ccc(-n5c6ccccc6c6cc7ccccc7cc65)cc4)ccc4ccccc34)n2)cc1 ; c1ccc(-c2cc(-c3ccc(-c4ccc5ccccc5c4-c4nc(-c5ccccc5)nc(-c5ccccc5)n4)cc3)nc(-c3ccc4ccccc4c3)n2)cc1 ; c1ccc(-c2cc(-c3cccc4ccccc34)nc(-c3ccc(-c4ccc5ccccc5c4-c4nc(-c5ccccc5)nc(-c5ccccc5)n4)cc3)n2)cc1 ; CC1(C)c2cc(-c3ccc(-c4ccc5ccccc5c4-c4nc(-c5ccccc5)nc(-c5ccccc5)n4)cc3)ccc2-c2c(C#N)cccc21 ; c1ccc(-c2nc(-c3ccccc3)nc(-c3c(-c4ccc(-c5ccc6oc7ccc8ccccc8c7c6c5)cc4)ccc4ccccc34)n2)cc1]

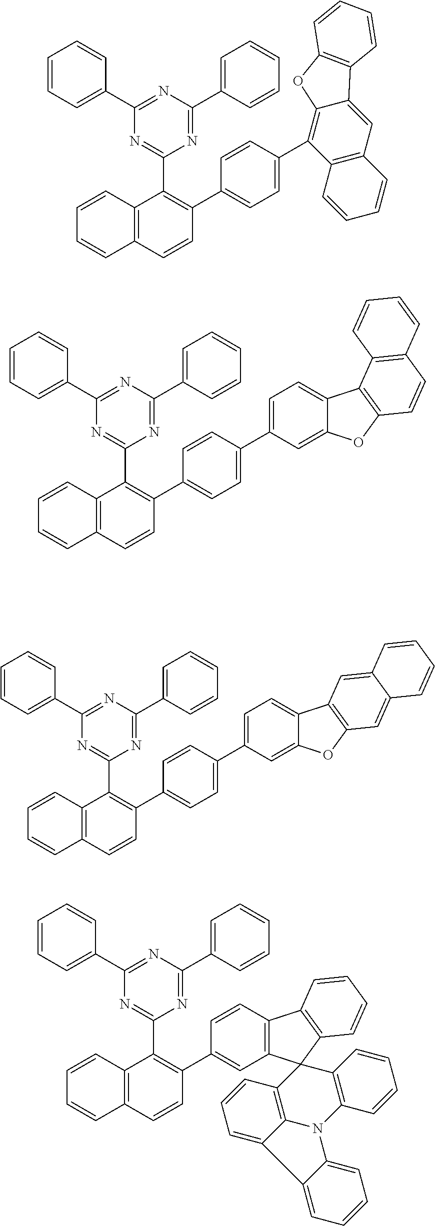 [SMILES: c1ccc(-c2nc(-c3ccccc3)nc(-c3c(-c4ccc(-c5c6ccccc6cc6c5oc5ccccc56)cc4)ccc4ccccc34)n2)cc1 ; c1ccc(-c2nc(-c3ccccc3)nc(-c3c(-c4ccc(-c5ccc6c(c5)oc5ccc7ccccc7c56)cc4)ccc4ccccc34)n2)cc1 ; c1ccc(-c2nc(-c3ccccc3)nc(-c3c(-c4ccc(-c5ccc6c(c5)oc5cc7ccccc7cc56)cc4)ccc4ccccc34)n2)cc1 ; c1ccc(-c2nc(-c3ccccc3)nc(-c3c(-c4ccc5c(c4)C4(c6ccccc6-5)c5ccccc5-n5c6ccccc6c6cccc4c65)ccc4ccccc34)n2)cc1]

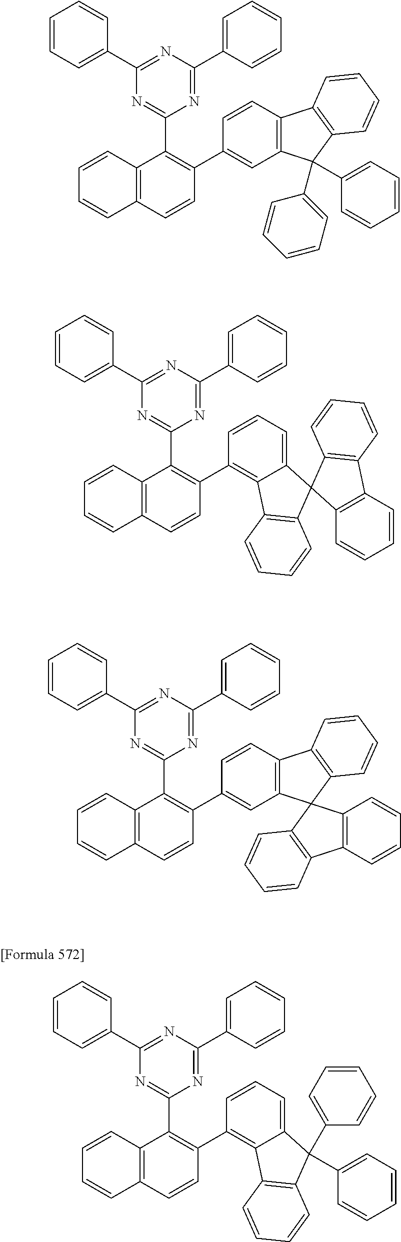 [SMILES: c1ccc(-c2nc(-c3ccccc3)nc(-c3c(-c4ccc5c(c4)C(c4ccccc4)(c4ccccc4)c4ccccc4-5)ccc4ccccc34)n2)cc1 ; c1ccc(-c2nc(-c3ccccc3)nc(-c3c(-c4cccc5c4-c4ccccc4C54c5ccccc5-c5ccccc54)ccc4ccccc34)n2)cc1 ; c1ccc(-c2nc(-c3ccccc3)nc(-c3c(-c4ccc5c(c4)C4(c6ccccc6-c6ccccc64)c4ccccc4-5)ccc4ccccc34)n2)cc1 ; c1ccc(-c2nc(-c3ccccc3)nc(-c3c(-c4cccc5c4-c4ccccc4C5(c4ccccc4)c4ccccc4)ccc4ccccc34)n2)cc1]

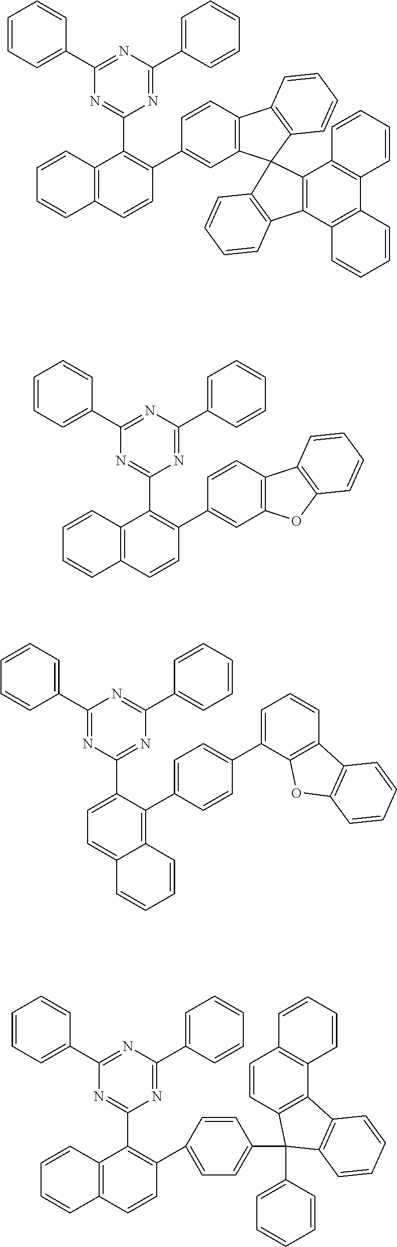 [SMILES: c1ccc(-c2nc(-c3ccccc3)nc(-c3c(-c4ccc5c(c4)C4(c6ccccc6-5)c5ccccc5-c5c4c4ccccc4c4ccccc54)ccc4ccccc34)n2)cc1 ; c1ccc(-c2nc(-c3ccccc3)nc(-c3c(-c4ccc5c(c4)oc4ccccc45)ccc4ccccc34)n2)cc1 ; c1ccc(-c2nc(-c3ccccc3)nc(-c3ccc4ccccc4c3-c3ccc(-c4cccc5c4oc4ccccc45)cc3)n2)cc1 ; c1ccc(-c2nc(-c3ccccc3)nc(-c3c(-c4ccc(C5(c6ccccc6)c6ccccc6-c6c5ccc5ccccc65)cc4)ccc4ccccc34)n2)cc1]

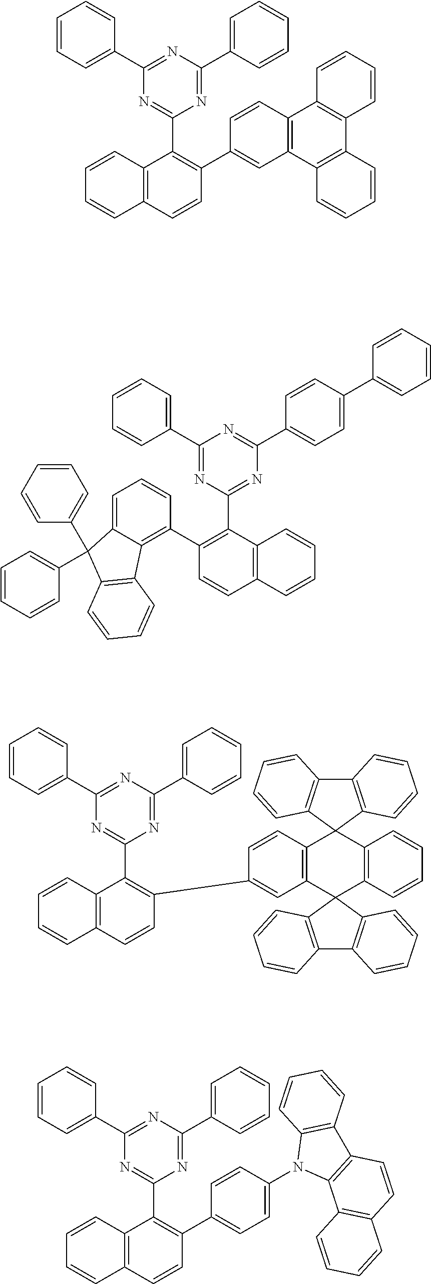 [SMILES: c1ccc(-c2nc(-c3ccccc3)nc(-c3c(-c4ccc5c6ccccc6c6ccccc6c5c4)ccc4ccccc34)n2)cc1 ; c1ccc(-c2ccc(-c3nc(-c4ccccc4)nc(-c4c(-c5cccc6c5-c5ccccc5C6(c5ccccc5)c5ccccc5)ccc5ccccc45)n3)cc2)cc1 ; c1ccc(-c2nc(-c3ccccc3)nc(-c3c(-c4ccc5c(c4)C4(c6ccccc6-c6ccccc64)c4ccccc4C54c5ccccc5-c5ccccc54)ccc4ccccc34)n2)cc1 ; c1ccc(-c2nc(-c3ccccc3)nc(-c3c(-c4ccc(-n5c6ccccc6c6ccc7ccccc7c65)cc4)ccc4ccccc34)n2)cc1]

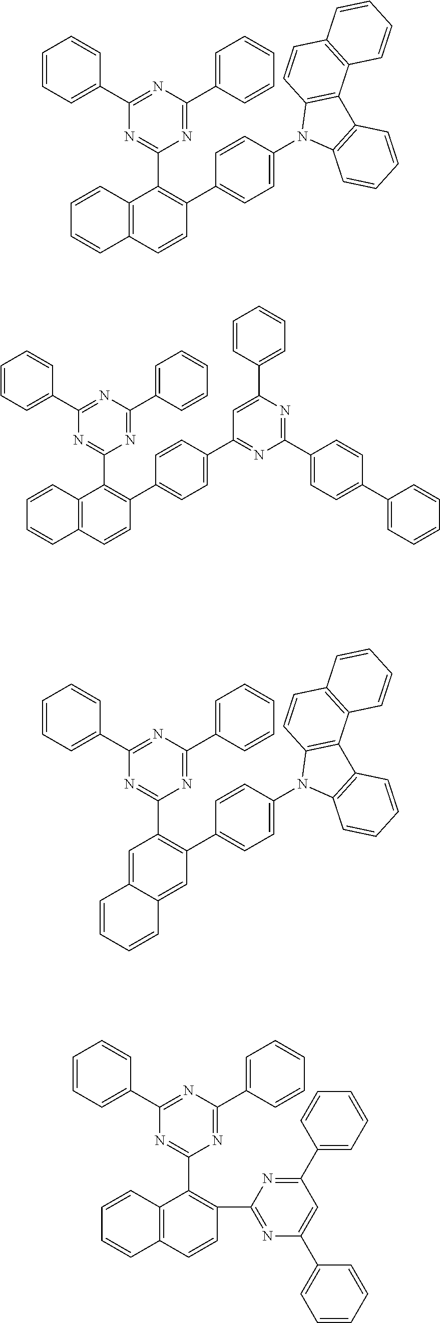 [SMILES: c1ccc(-c2nc(-c3ccccc3)nc(-c3c(-c4ccc(-n5c6ccccc6c6c7ccccc7ccc65)cc4)ccc4ccccc34)n2)cc1 ; c1ccc(-c2ccc(-c3nc(-c4ccccc4)cc(-c4ccc(-c5ccc6ccccc6c5-c5nc(-c6ccccc6)nc(-c6ccccc6)n5)cc4)n3)cc2)cc1 ; c1ccc(-c2nc(-c3ccccc3)nc(-c3cc4ccccc4cc3-c3ccc(-n4c5ccccc5c5c6ccccc6ccc54)cc3)n2)cc1 ; c1ccc(-c2cc(-c3ccccc3)nc(-c3ccc4ccccc4c3-c3nc(-c4ccccc4)nc(-c4ccccc4)n3)n2)cc1]

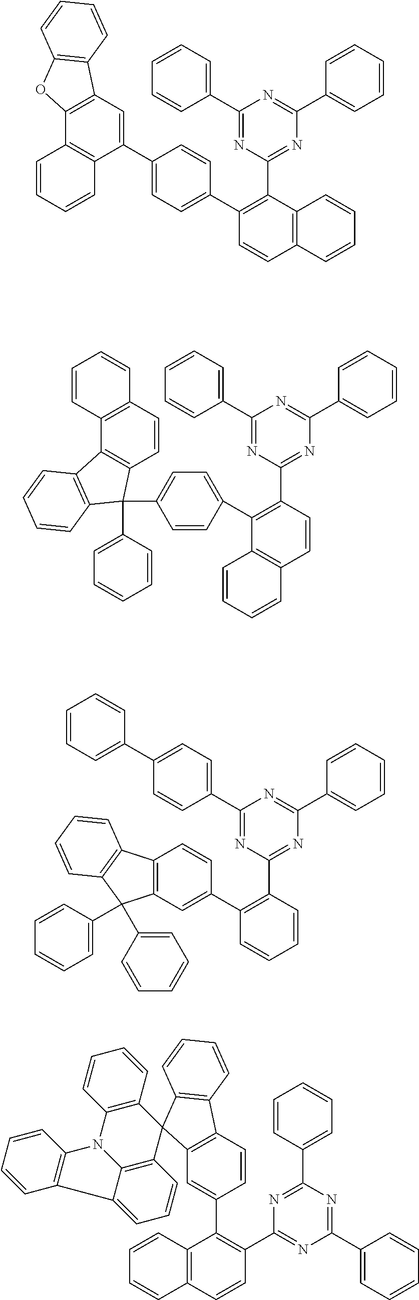 [SMILES: c1ccc(-c2nc(-c3ccccc3)nc(-c3c(-c4ccc(-c5cc6c7ccccc7oc6c6ccccc56)cc4)ccc4ccccc34)n2)cc1 ; c1ccc(-c2nc(-c3ccccc3)nc(-c3ccc4ccccc4c3-c3ccc(C4(c5ccccc5)c5ccccc5-c5c4ccc4ccccc54)cc3)n2)cc1 ; c1ccc(-c2ccc(-c3nc(-c4ccccc4)nc(-c4ccccc4-c4ccc5c(c4)C(c4ccccc4)(c4ccccc4)c4ccccc4-5)n3)cc2)cc1 ; c1ccc(-c2nc(-c3ccccc3)nc(-c3ccc4ccccc4c3-c3ccc4c(c3)C3(c5ccccc5-4)c4ccccc4-n4c5ccccc5c5cccc3c54)n2)cc1]

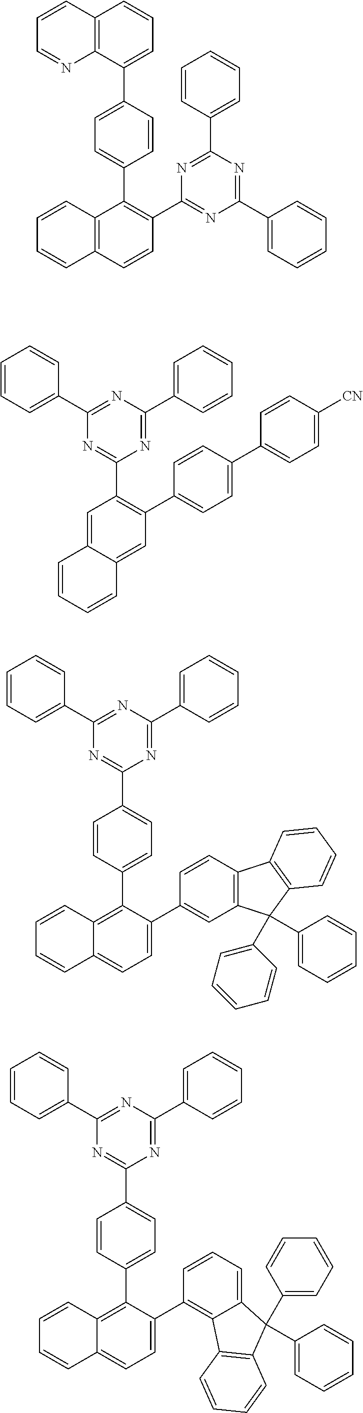 [SMILES: c1ccc(-c2nc(-c3ccccc3)nc(-c3ccc4ccccc4c3-c3ccc(-c4cccc5cccnc45)cc3)n2)cc1 ; N#Cc1ccc(-c2ccc(-c3cc4ccccc4cc3-c3nc(-c4ccccc4)nc(-c4ccccc4)n3)cc2)cc1 ; c1ccc(-c2nc(-c3ccccc3)nc(-c3ccc(-c4c(-c5ccc6c(c5)C(c5ccccc5)(c5ccccc5)c5ccccc5-6)ccc5ccccc45)cc3)n2)cc1 ; c1ccc(-c2nc(-c3ccccc3)nc(-c3ccc(-c4c(-c5cccc6c5-c5ccccc5C6(c5ccccc5)c5ccccc5)ccc5ccccc45)cc3)n2)cc1]

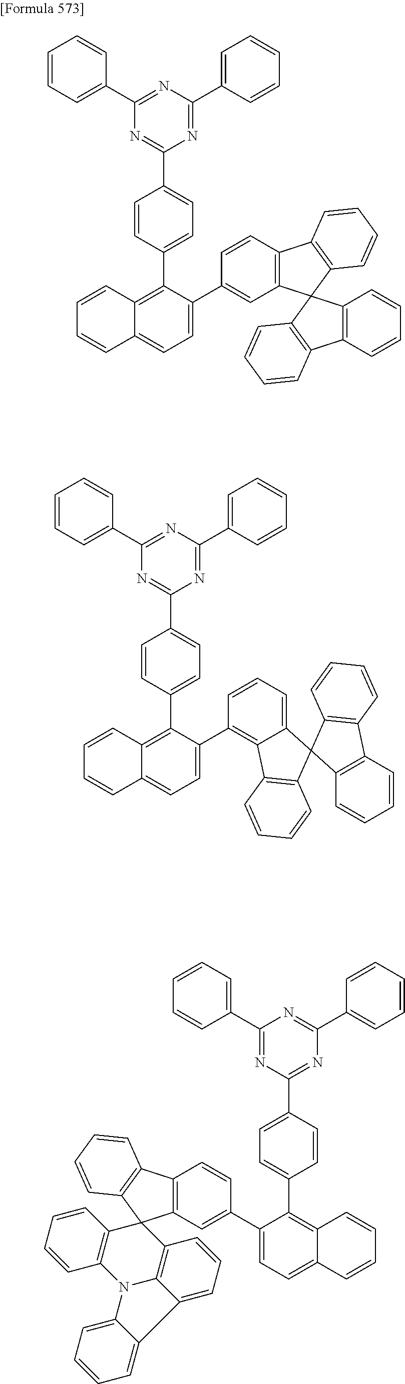 [SMILES: c1ccc(-c2nc(-c3ccccc3)nc(-c3ccc(-c4c(-c5ccc6c(c5)C5(c7ccccc7-c7ccccc75)c5ccccc5-6)ccc5ccccc45)cc3)n2)cc1 ; c1ccc(-c2nc(-c3ccccc3)nc(-c3ccc(-c4c(-c5cccc6c5-c5ccccc5C65c6ccccc6-c6ccccc65)ccc5ccccc45)cc3)n2)cc1 ; c1ccc(-c2nc(-c3ccccc3)nc(-c3ccc(-c4c(-c5ccc6c(c5)C5(c7ccccc7-6)c6ccccc6-n6c7ccccc7c7cccc5c76)ccc5ccccc45)cc3)n2)cc1]

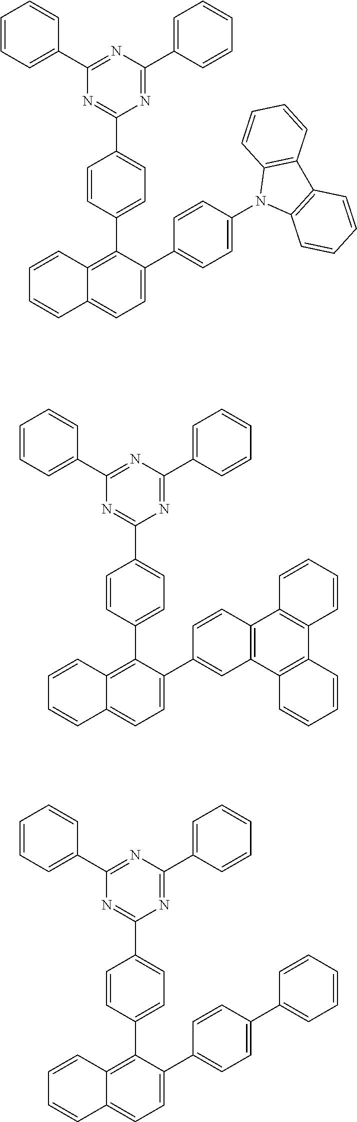 [SMILES: c1ccc(-c2nc(-c3ccccc3)nc(-c3ccc(-c4c(-c5ccc(-n6c7ccccc7c7ccccc76)cc5)ccc5ccccc45)cc3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2ccc(-c3ccc4ccccc4c3-c3ccc(-c4nc(-c5ccccc5)nc(-c5ccccc5)n4)cc3)cc2)cc1]

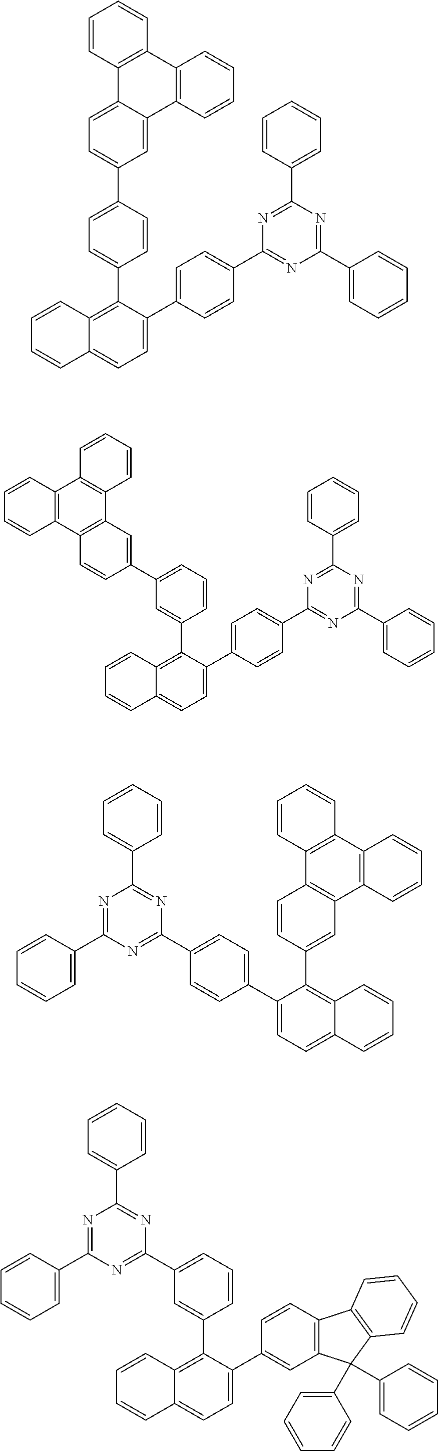 [SMILES: c1ccc(-c2nc(-c3ccccc3)nc(-c3ccc(-c4ccc5ccccc5c4-c4ccc(-c5ccc6c7ccccc7c7ccccc7c6c5)cc4)cc3)n2)cc1 ; c1ccc(-c2nc(-c3ccccc3)nc(-c3ccc(-c4ccc5ccccc5c4-c4cccc(-c5ccc6c7ccccc7c7ccccc7c6c5)c4)cc3)n2)cc1 ; c1ccc(-c2nc(-c3ccccc3)nc(-c3ccc(-c4ccc5ccccc5c4-c4ccc5c6ccccc6c6ccccc6c5c4)cc3)n2)cc1 ; c1ccc(-c2nc(-c3ccccc3)nc(-c3cccc(-c4c(-c5ccc6c(c5)C(c5ccccc5)(c5ccccc5)c5ccccc5-6)ccc5ccccc45)c3)n2)cc1]

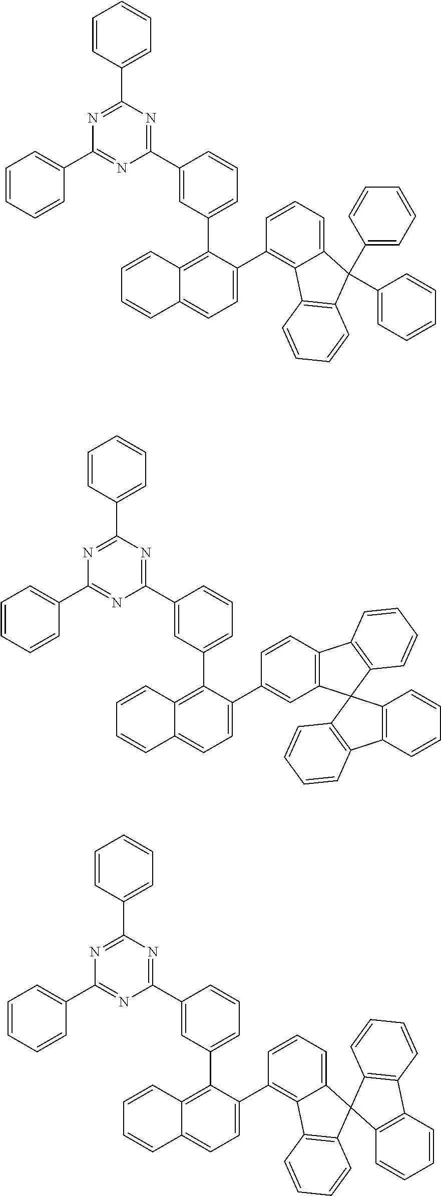 [SMILES: c1ccc(-c2nc(-c3ccccc3)nc(-c3cccc(-c4c(-c5cccc6c5-c5ccccc5C6(c5ccccc5)c5ccccc5)ccc5ccccc45)c3)n2)cc1 ; c1ccc(-c2nc(-c3ccccc3)nc(-c3cccc(-c4c(-c5ccc6c(c5)C5(c7ccccc7-c7ccccc75)c5ccccc5-6)ccc5ccccc45)c3)n2)cc1 ; c1ccc(-c2nc(-c3ccccc3)nc(-c3cccc(-c4c(-c5cccc6c5-c5ccccc5C65c6ccccc6-c6ccccc65)ccc5ccccc45)c3)n2)cc1]

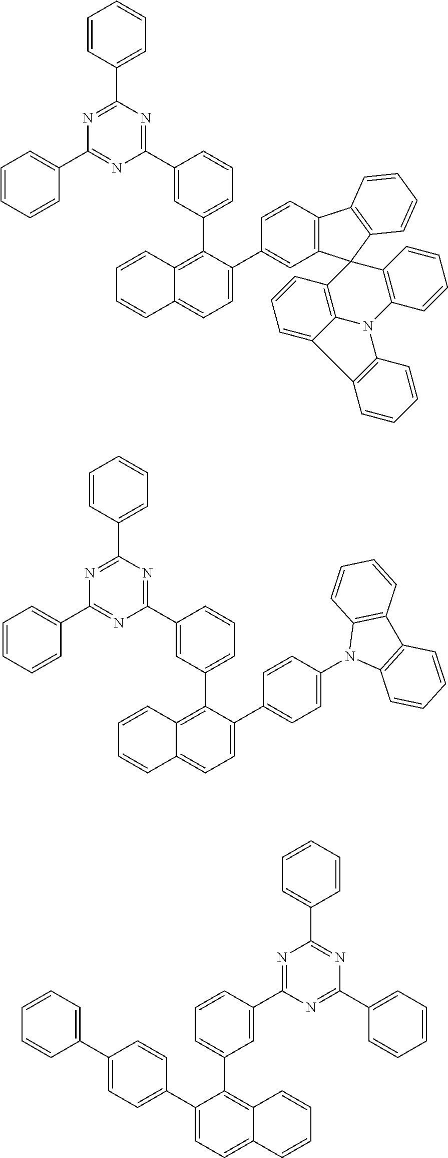 [SMILES: c1ccc(-c2nc(-c3ccccc3)nc(-c3cccc(-c4c(-c5ccc6c(c5)C5(c7ccccc7-6)c6ccccc6-n6c7ccccc7c7cccc5c76)ccc5ccccc45)c3)n2)cc1 ; c1ccc(-c2nc(-c3ccccc3)nc(-c3cccc(-c4c(-c5ccc(-n6c7ccccc7c7ccccc76)cc5)ccc5ccccc45)c3)n2)cc1 ; c1ccc(-c2ccc(-c3ccc4ccccc4c3-c3cccc(-c4nc(-c5ccccc5)nc(-c5ccccc5)n4)c3)cc2)cc1]

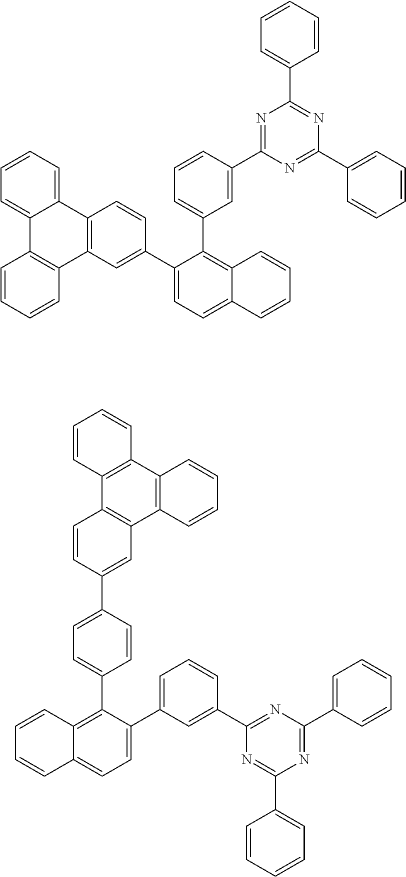 [SMILES: c1ccc(-c2nc(-c3ccccc3)nc(-c3cccc(-c4c(-c5ccc6c7ccccc7c7ccccc7c6c5)ccc5ccccc45)c3)n2)cc1 ; c1ccc(-c2nc(-c3ccccc3)nc(-c3cccc(-c4ccc5ccccc5c4-c4ccc(-c5ccc6c7ccccc7c7ccccc7c6c5)cc4)c3)n2)cc1]

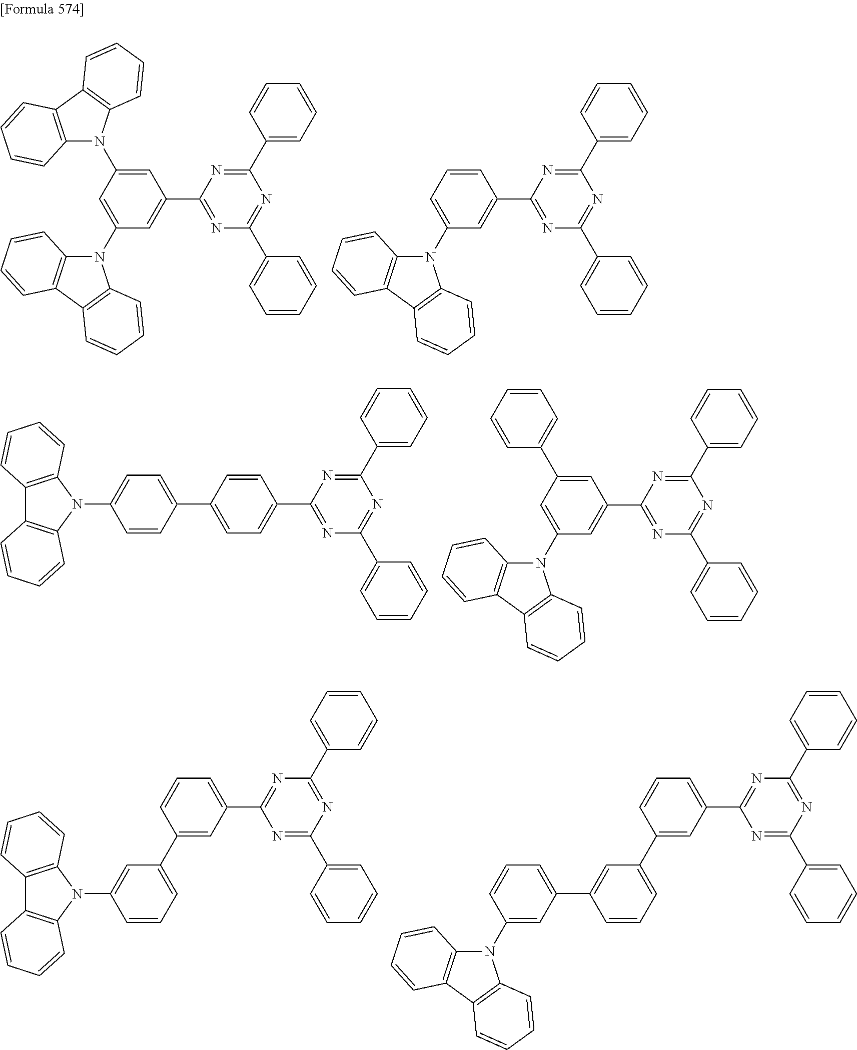

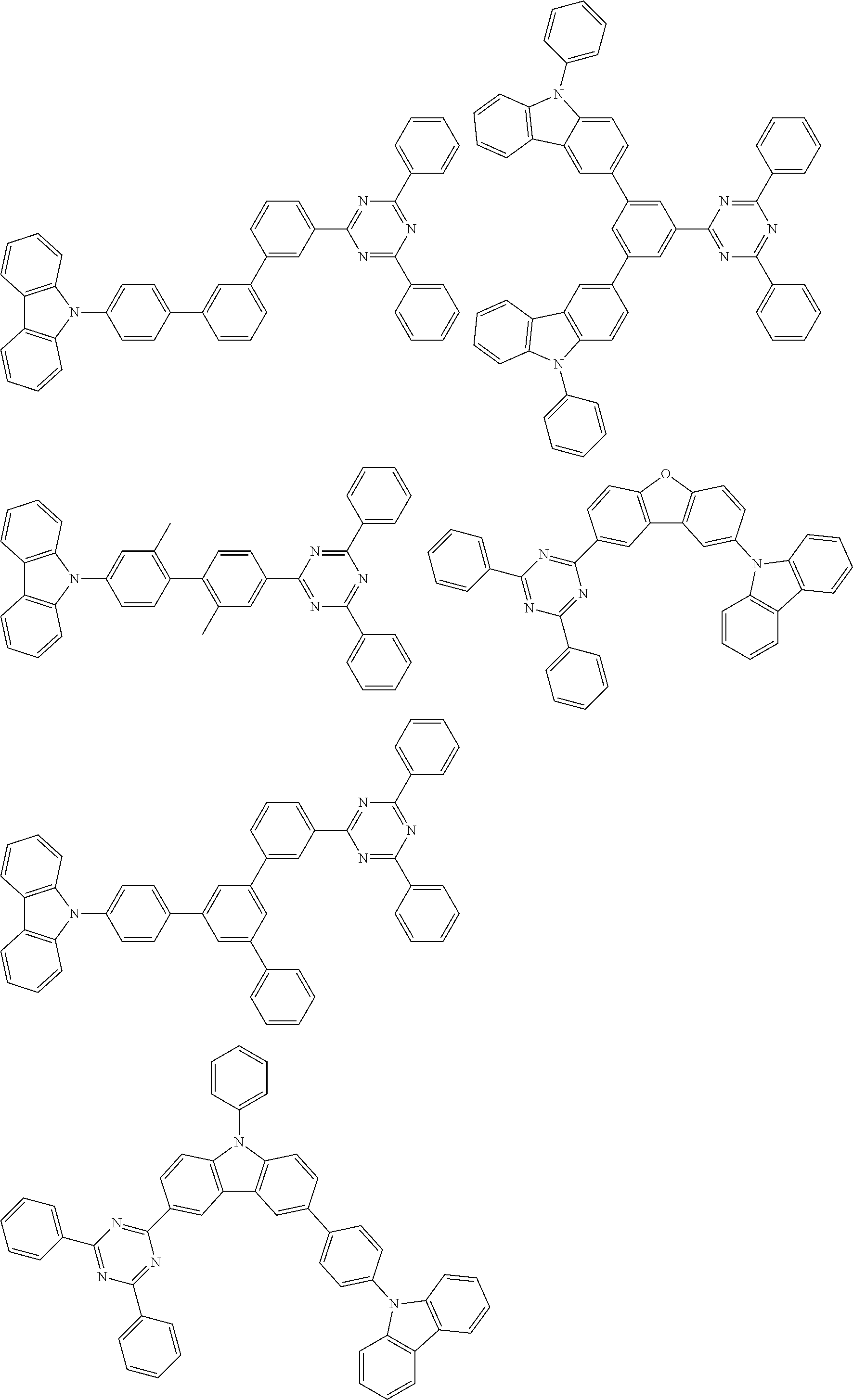

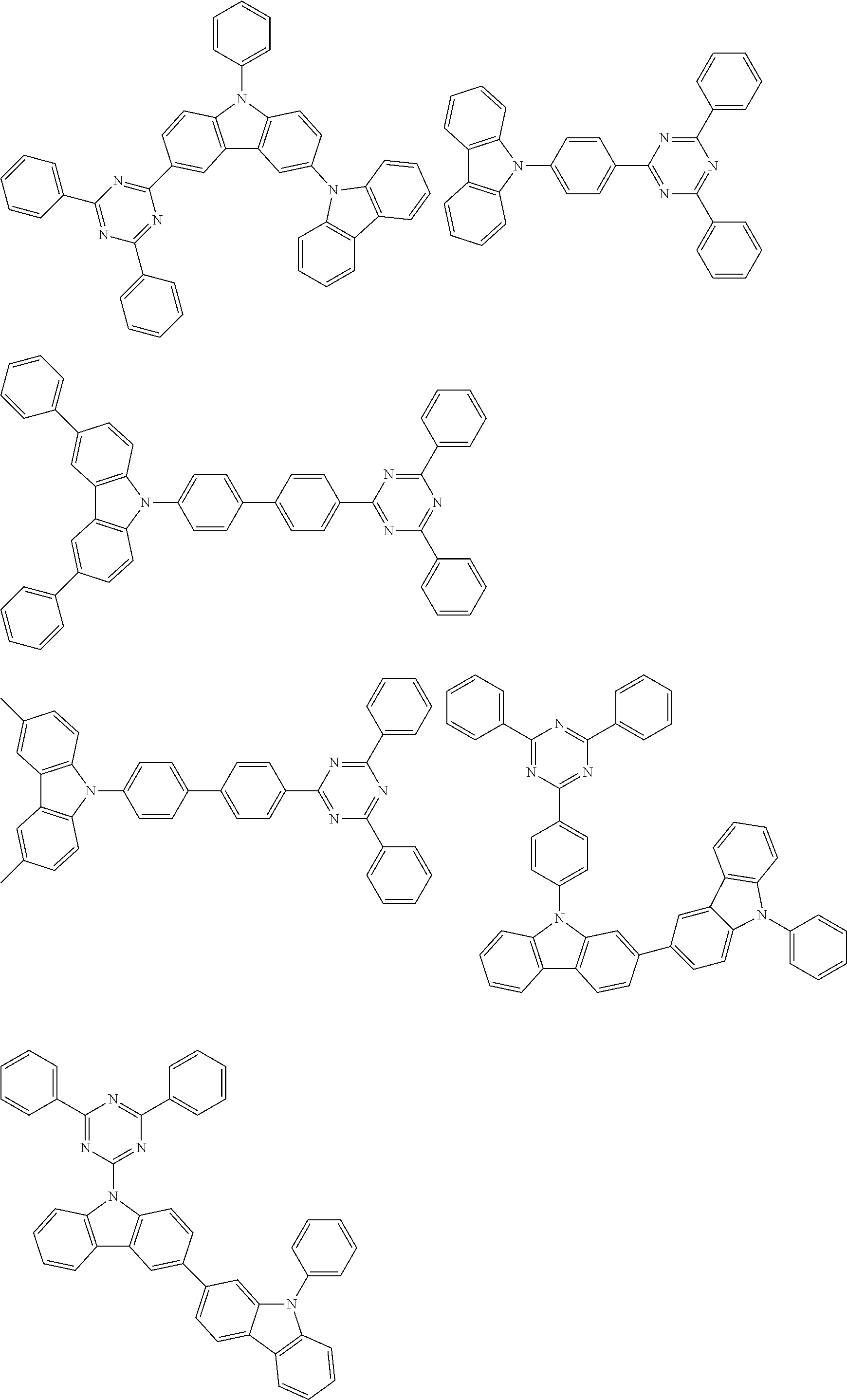

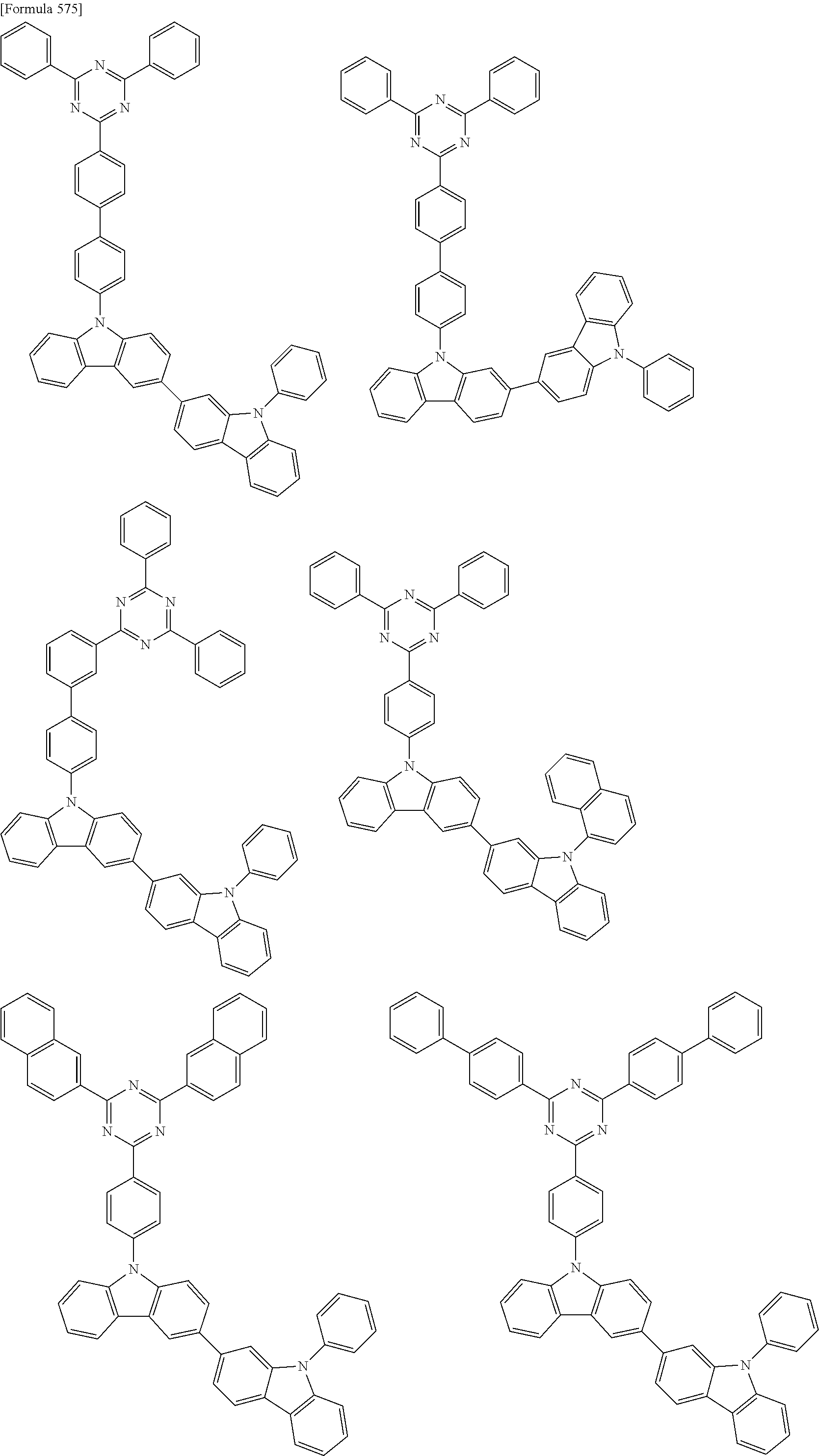

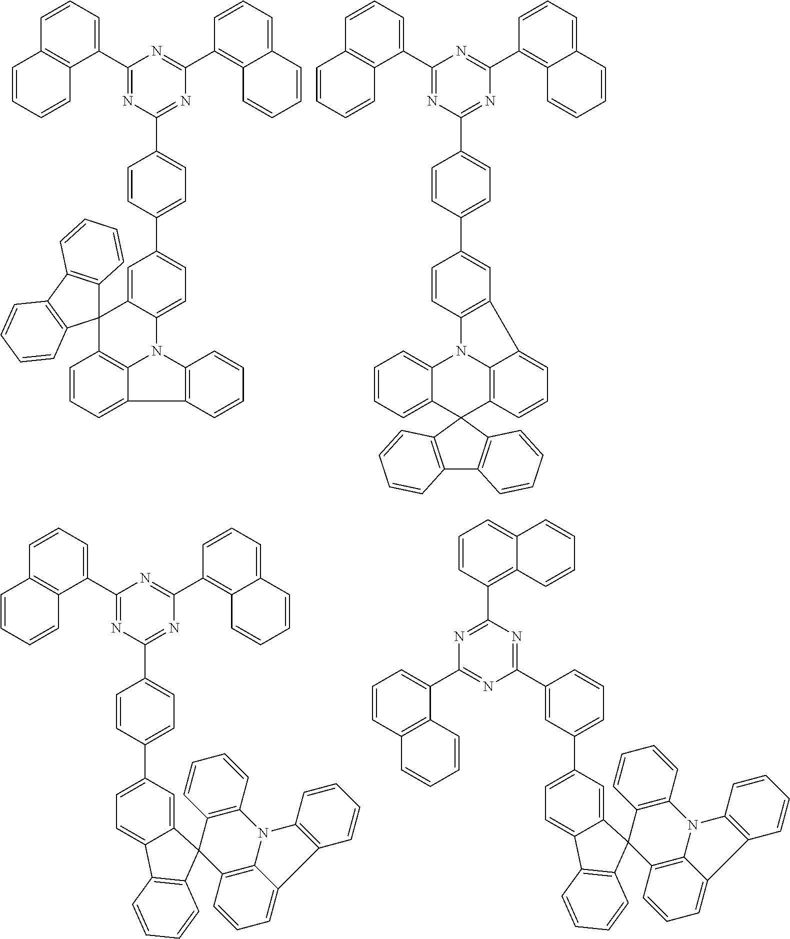

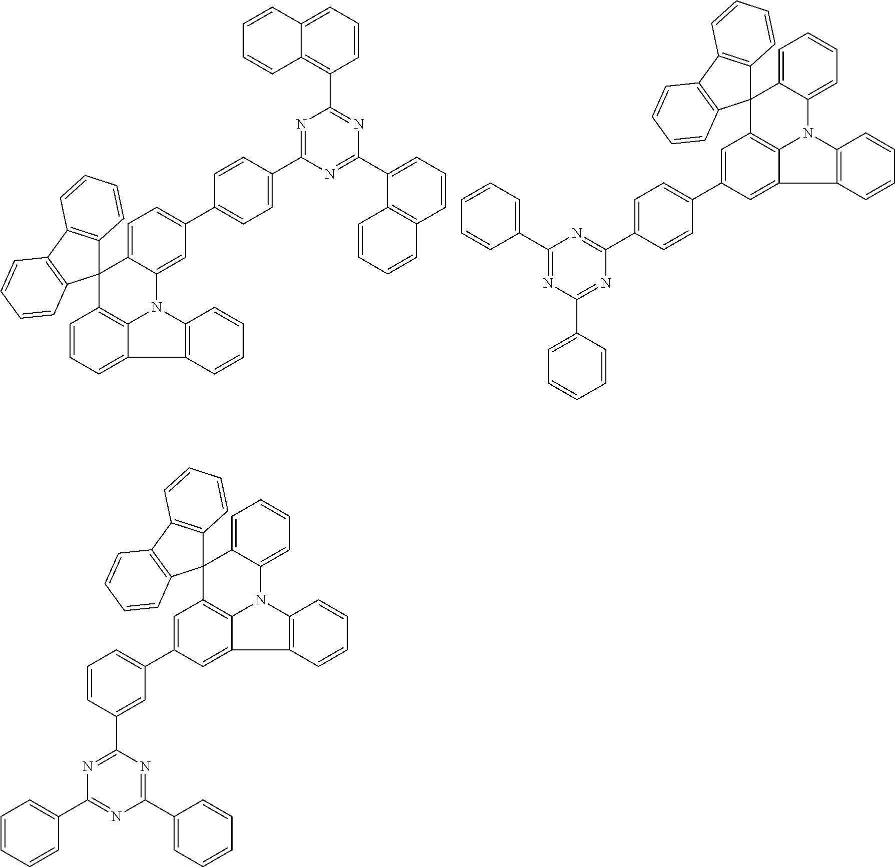

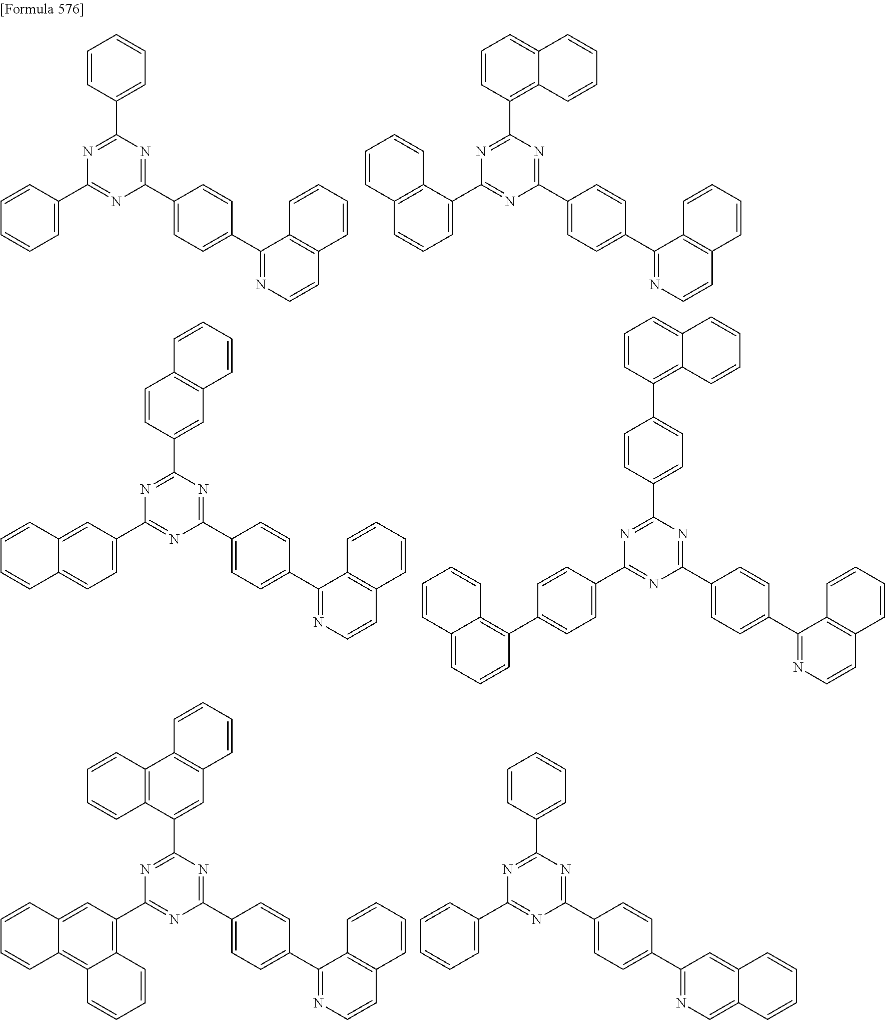

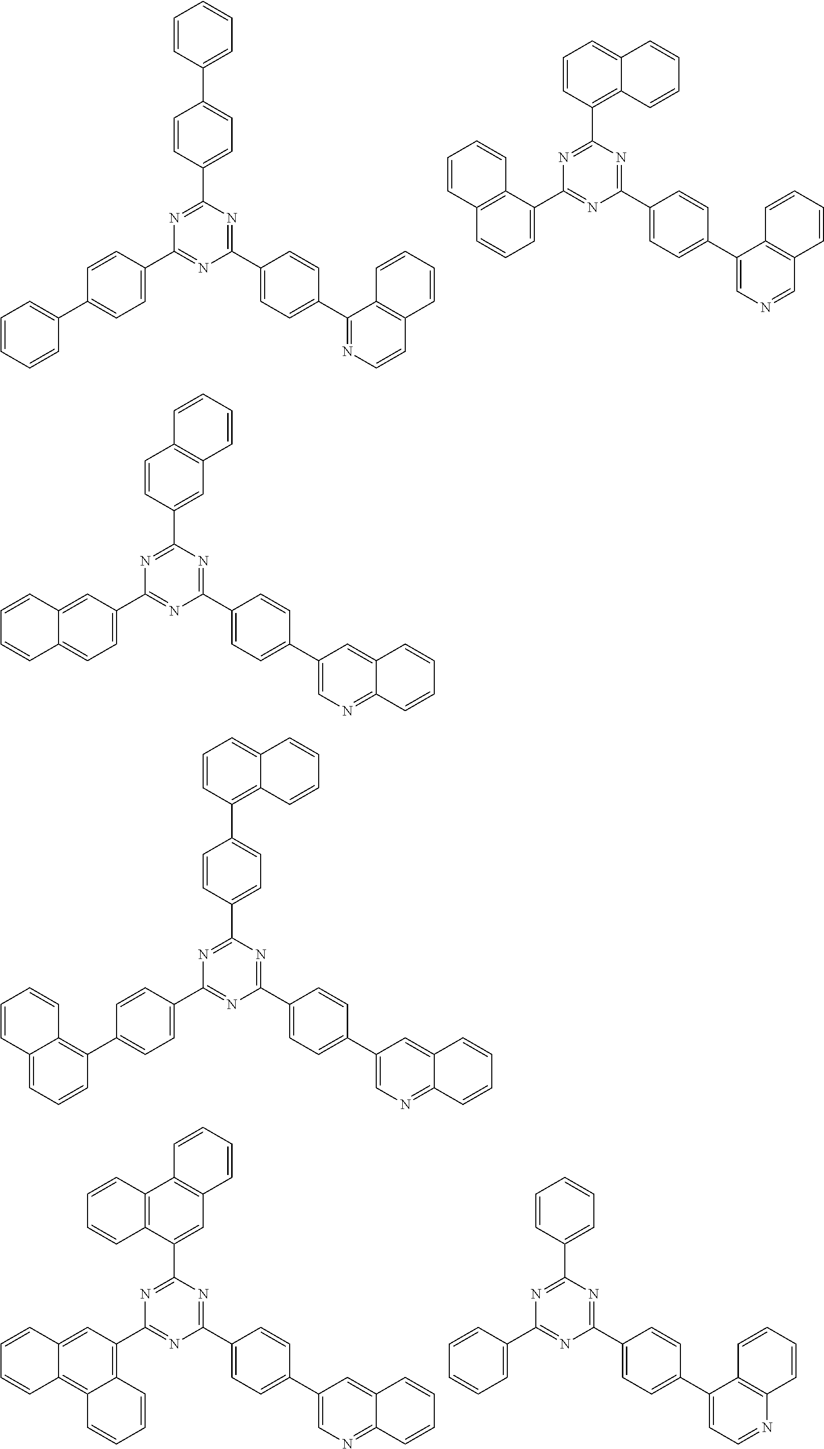

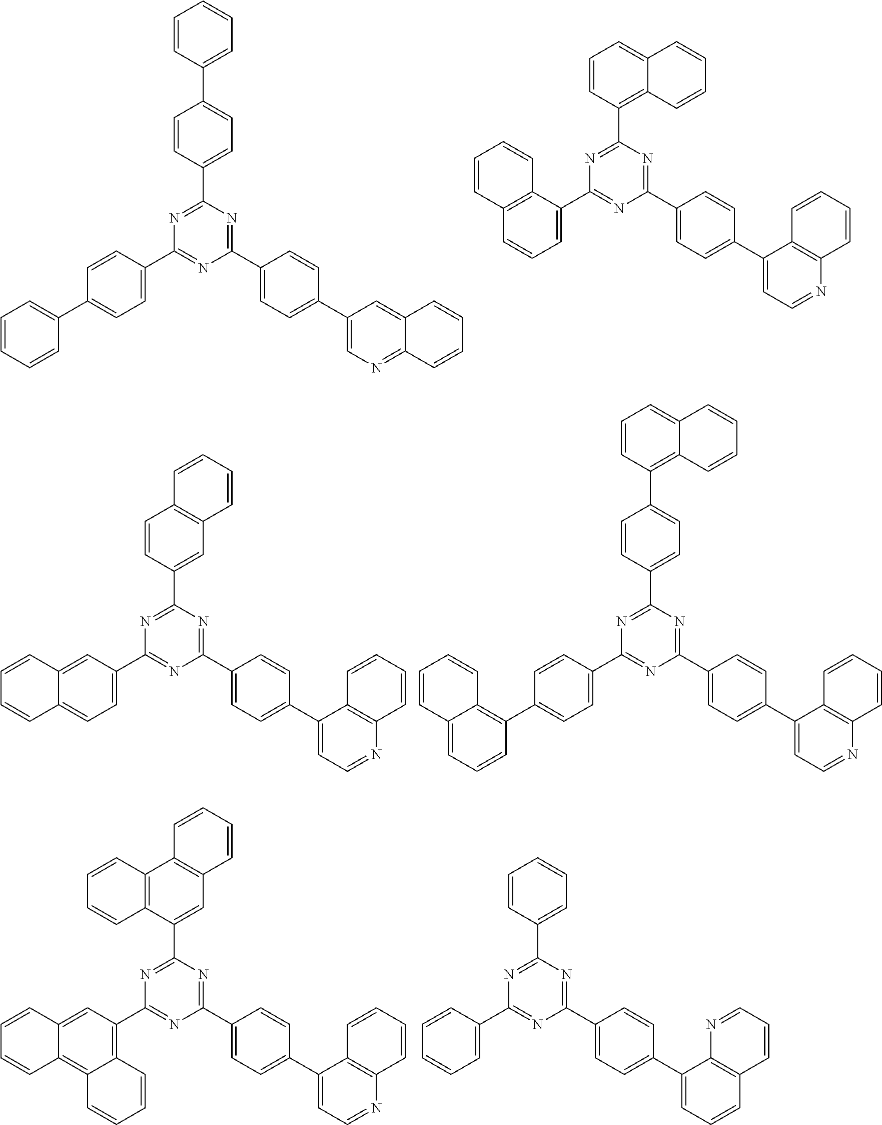

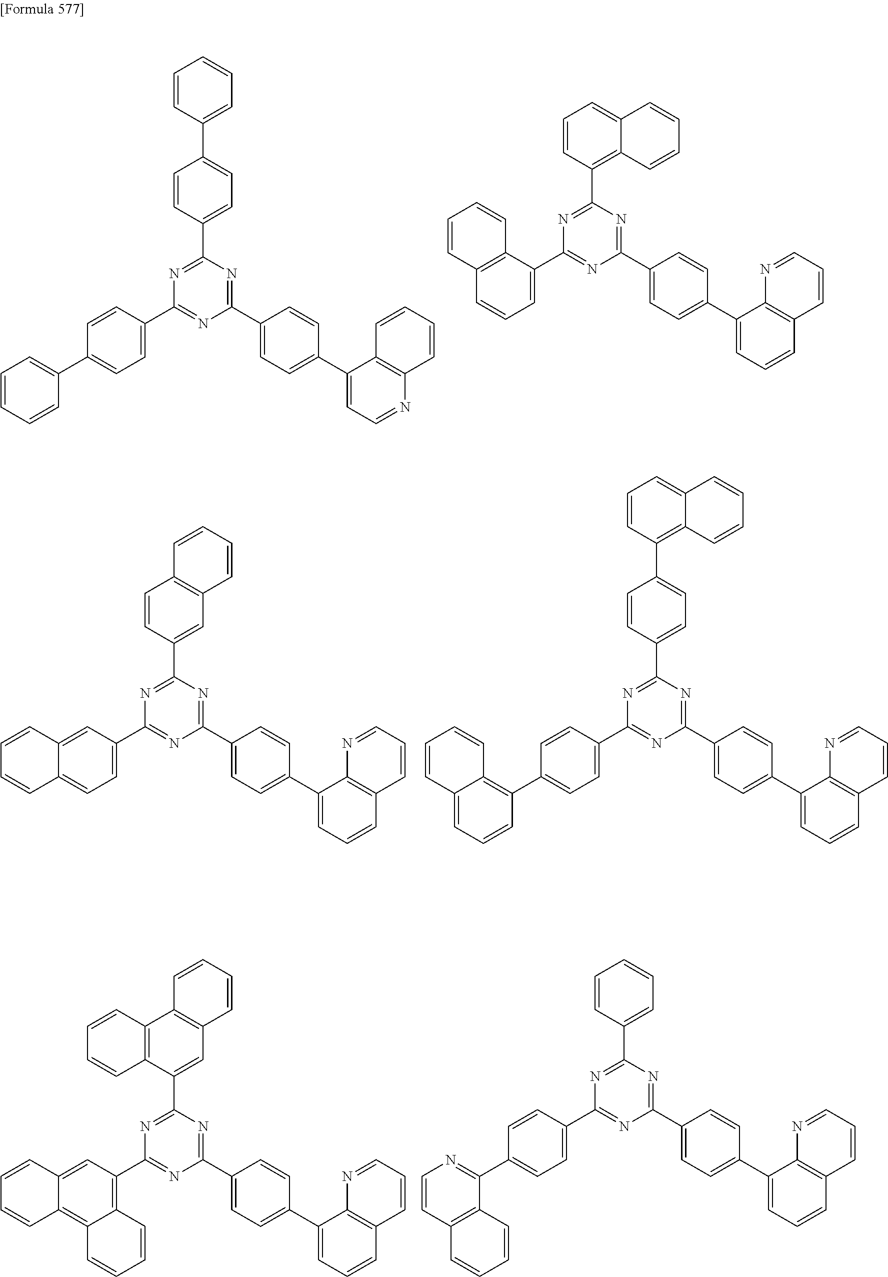

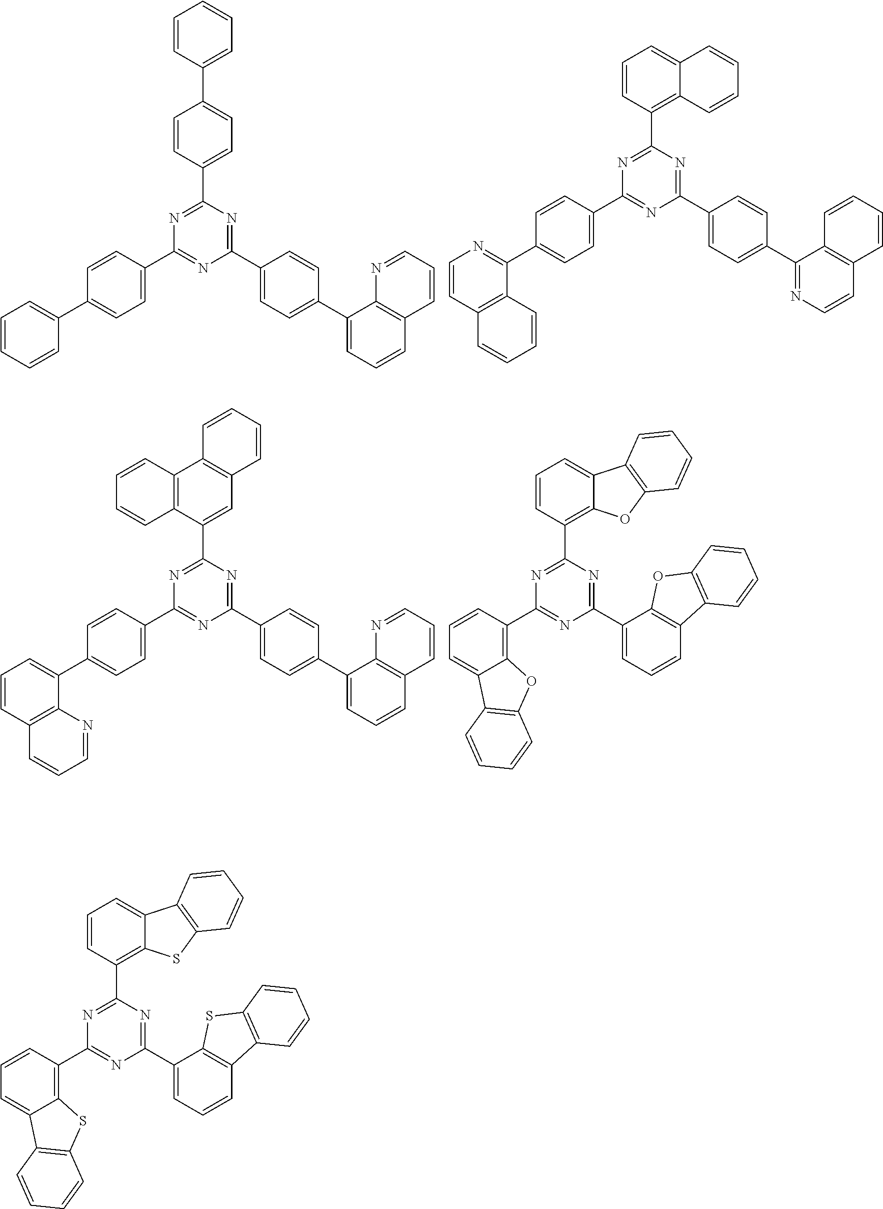

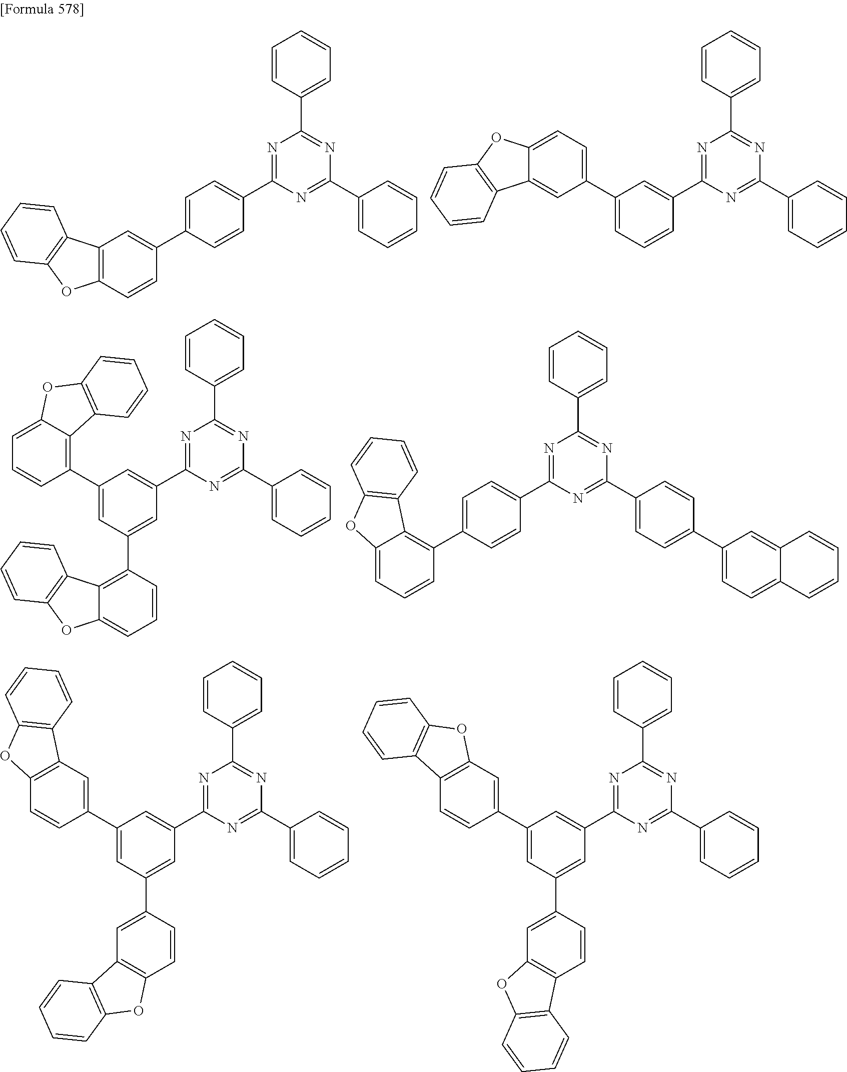

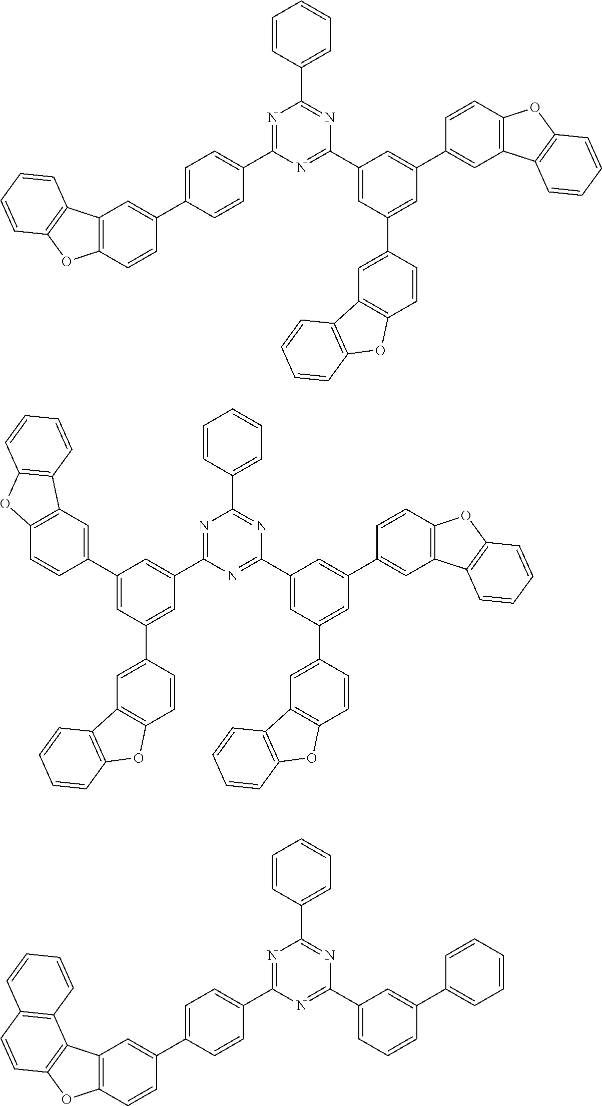 [SMILES: c1ccc(-c2nc(-c3ccc(-c4ccc5oc6ccccc6c5c4)cc3)nc(-c3cc(-c4ccc5oc6ccccc6c5c4)cc(-c4ccc5oc6ccccc6c5c4)c3)n2)cc1 ; c1ccc(-c2nc(-c3cc(-c4ccc5oc6ccccc6c5c4)cc(-c4ccc5oc6ccccc6c5c4)c3)nc(-c3cc(-c4ccc5oc6ccccc6c5c4)cc(-c4ccc5oc6ccccc6c5c4)c3)n2)cc1 ; c1ccc(-c2cccc(-c3nc(-c4ccccc4)nc(-c4ccc(-c5ccc6oc7ccc8ccccc8c7c6c5)cc4)n3)c2)cc1]

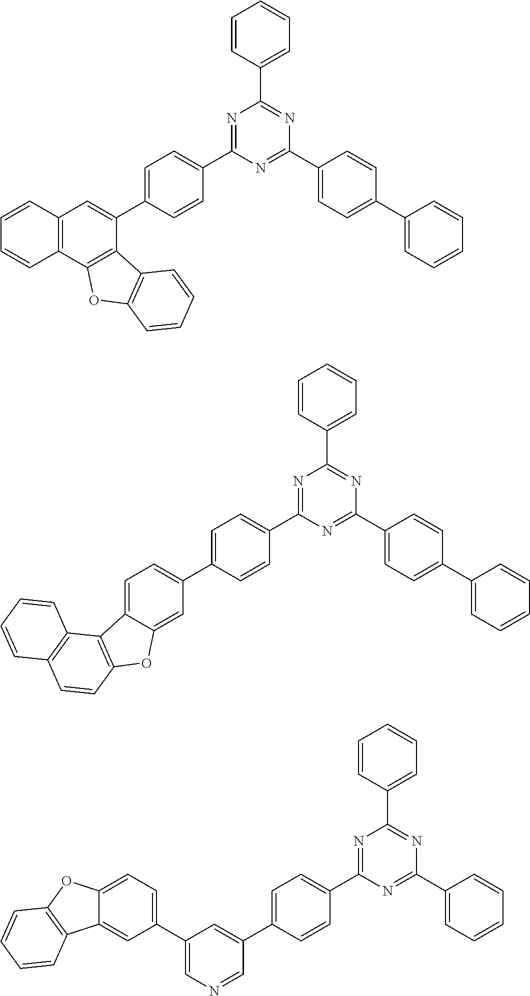 [SMILES: c1ccc(-c2ccc(-c3nc(-c4ccccc4)nc(-c4ccc(-c5cc6ccccc6c6oc7ccccc7c56)cc4)n3)cc2)cc1 ; c1ccc(-c2ccc(-c3nc(-c4ccccc4)nc(-c4ccc(-c5ccc6c(c5)oc5ccc7ccccc7c56)cc4)n3)cc2)cc1 ; c1ccc(-c2nc(-c3ccccc3)nc(-c3ccc(-c4cncc(-c5ccc6oc7ccccc7c6c5)c4)cc3)n2)cc1]

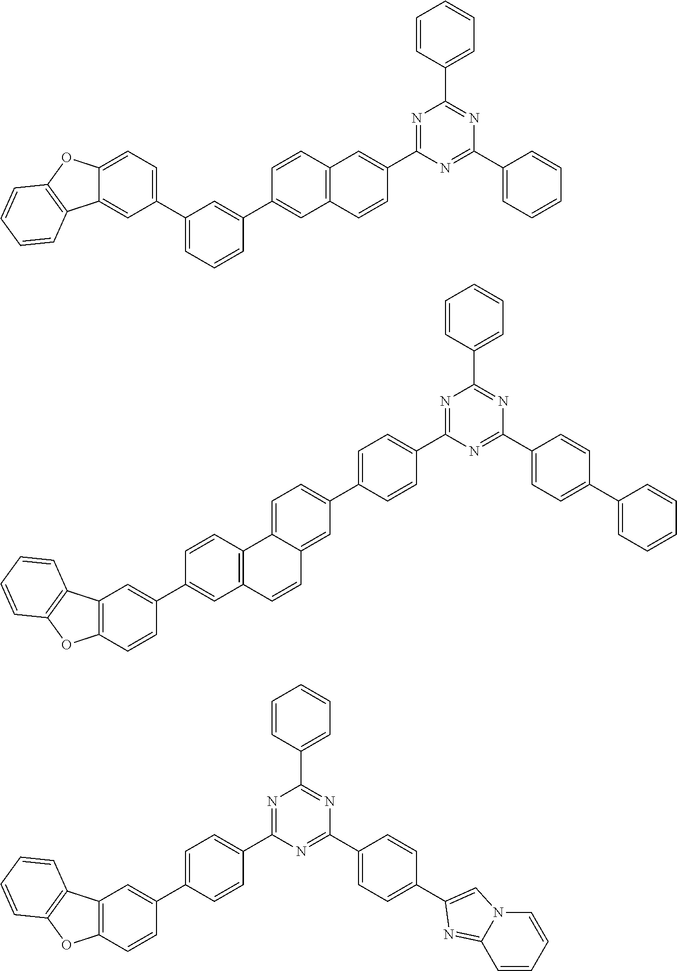 [SMILES: c1ccc(-c2nc(-c3ccccc3)nc(-c3ccc4cc(-c5cccc(-c6ccc7oc8ccccc8c7c6)c5)ccc4c3)n2)cc1 ; c1ccc(-c2ccc(-c3nc(-c4ccccc4)nc(-c4ccc(-c5ccc6c(ccc7cc(-c8ccc9oc%10ccccc%10c9c8)ccc76)c5)cc4)n3)cc2)cc1 ; c1ccc(-c2nc(-c3ccc(-c4ccc5oc6ccccc6c5c4)cc3)nc(-c3ccc(-c4cn5ccccc5n4)cc3)n2)cc1]

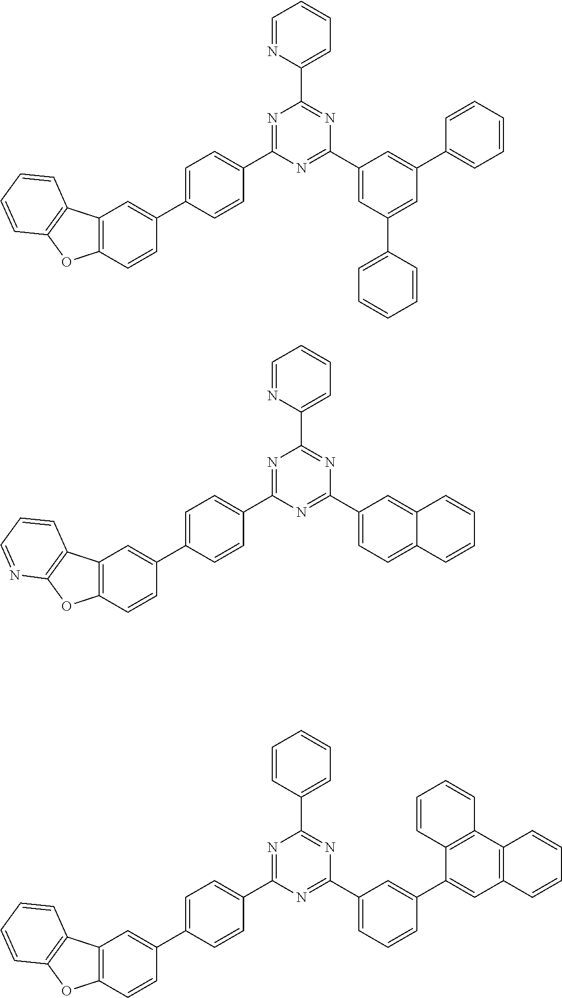 [SMILES: c1ccc(-c2cc(-c3ccccc3)cc(-c3nc(-c4ccc(-c5ccc6oc7ccccc7c6c5)cc4)nc(-c4ccccn4)n3)c2)cc1 ; c1ccc(-c2nc(-c3ccc(-c4ccc5oc6ncccc6c5c4)cc3)nc(-c3ccc4ccccc4c3)n2)nc1 ; c1ccc(-c2nc(-c3ccc(-c4ccc5oc6ccccc6c5c4)cc3)nc(-c3cccc(-c4cc5ccccc5c5ccccc45)c3)n2)cc1]

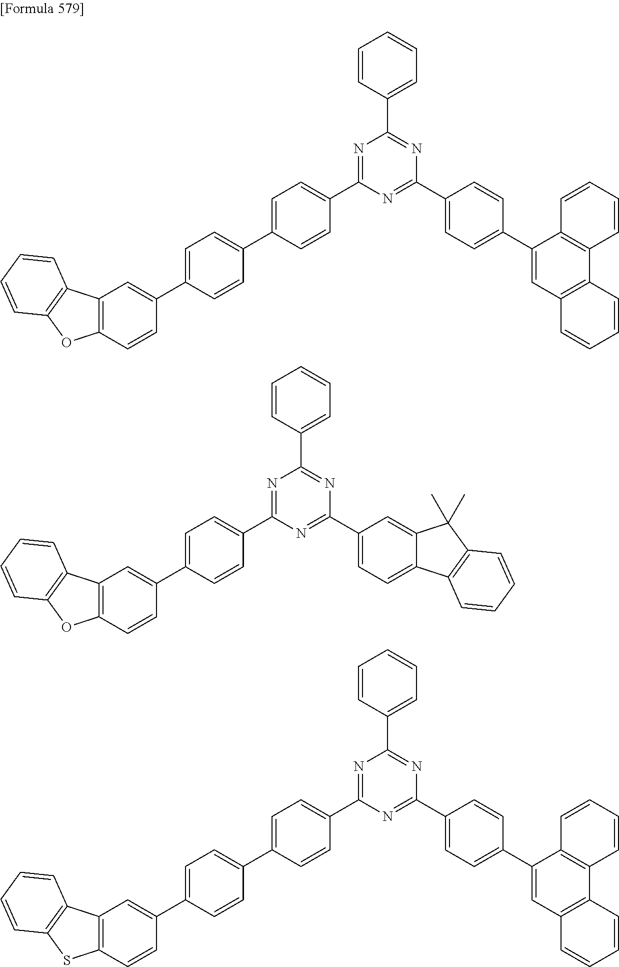 [SMILES: c1ccc(-c2nc(-c3ccc(-c4ccc(-c5ccc6oc7ccccc7c6c5)cc4)cc3)nc(-c3ccc(-c4cc5ccccc5c5ccccc45)cc3)n2)cc1 ; CC1(C)c2ccccc2-c2ccc(-c3nc(-c4ccccc4)nc(-c4ccc(-c5ccc6oc7ccccc7c6c5)cc4)n3)cc21 ; c1ccc(-c2nc(-c3ccc(-c4ccc(-c5ccc6sc7ccccc7c6c5)cc4)cc3)nc(-c3ccc(-c4cc5ccccc5c5ccccc45)cc3)n2)cc1]

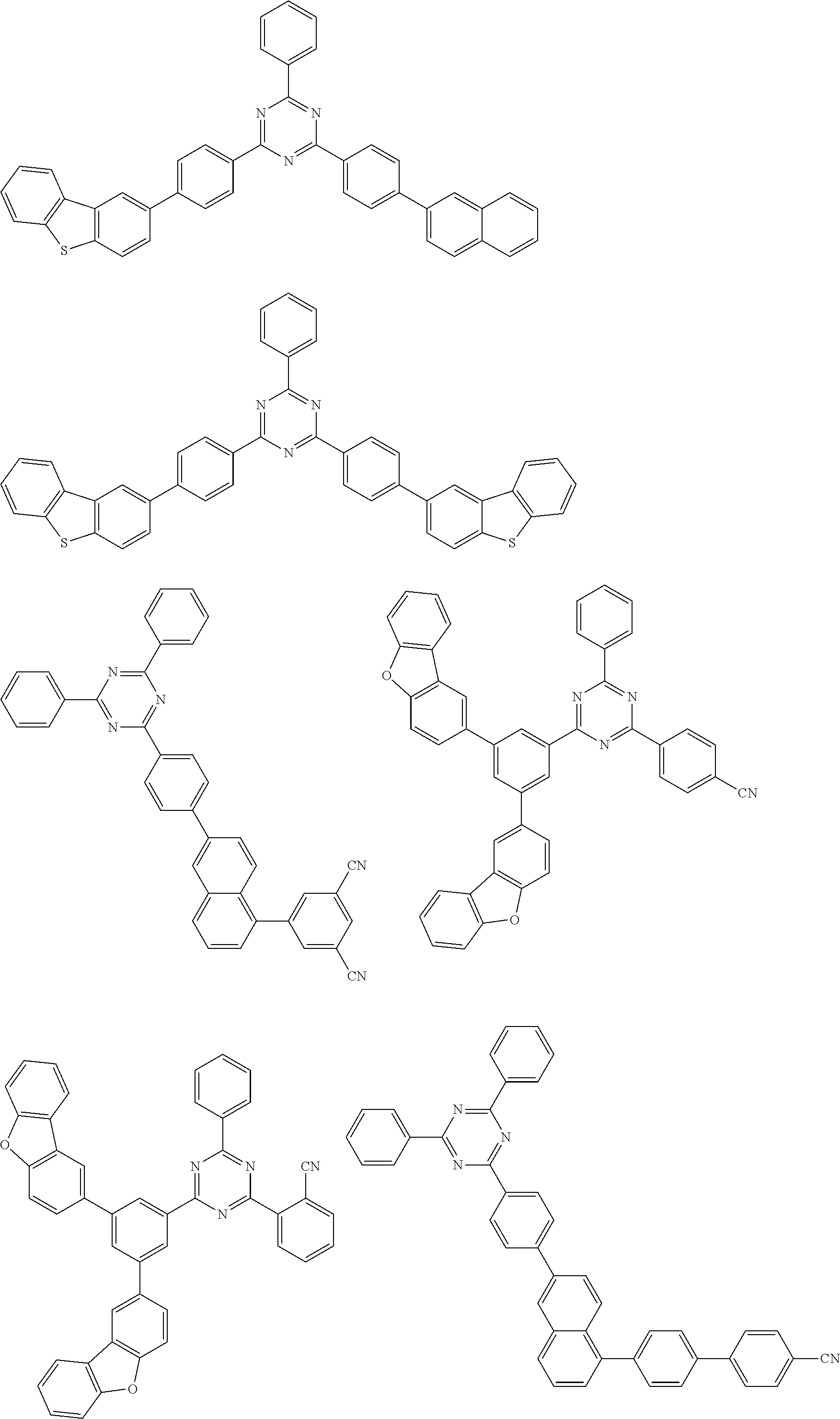

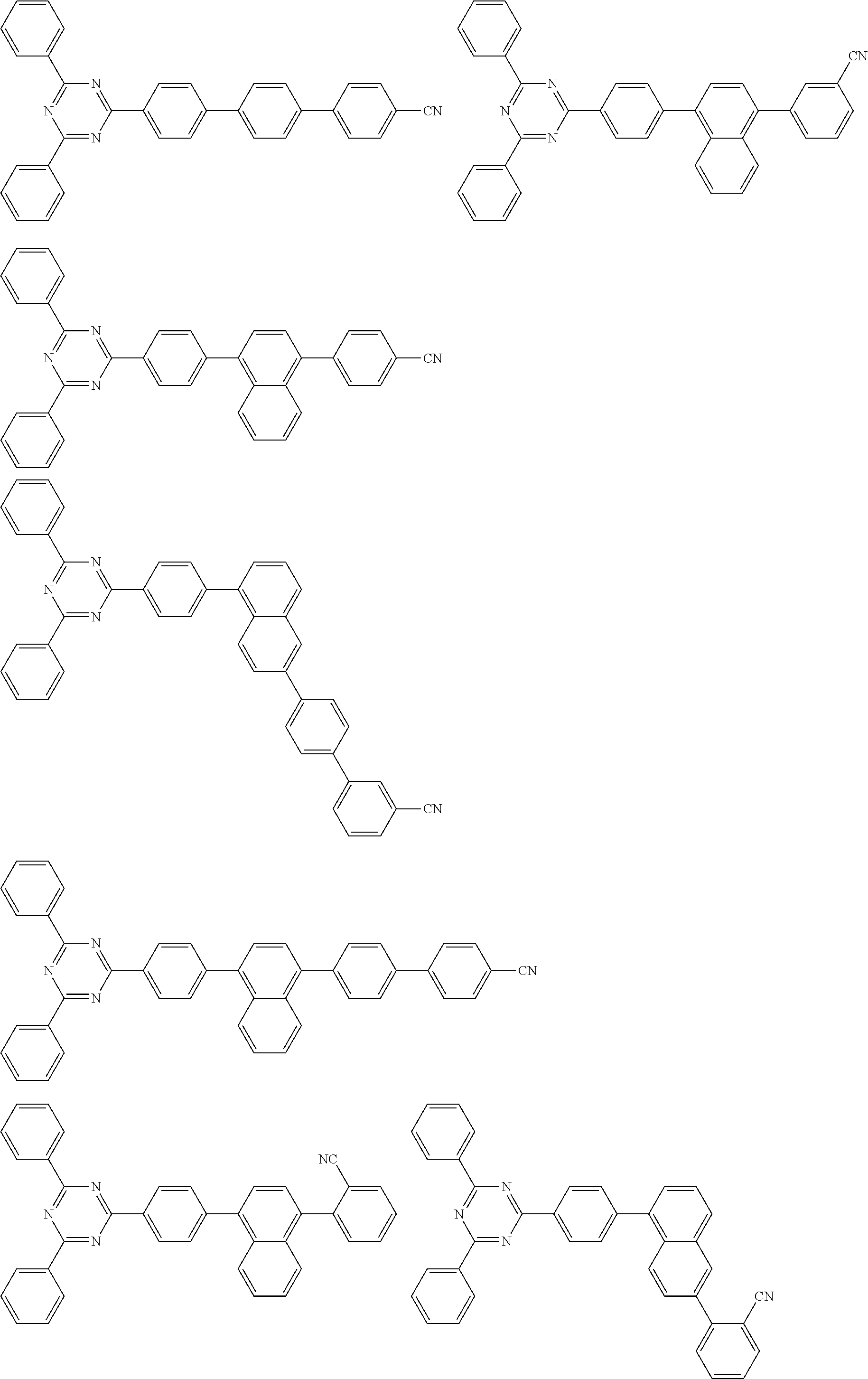

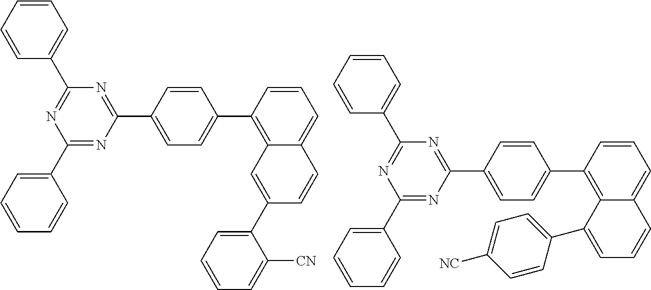

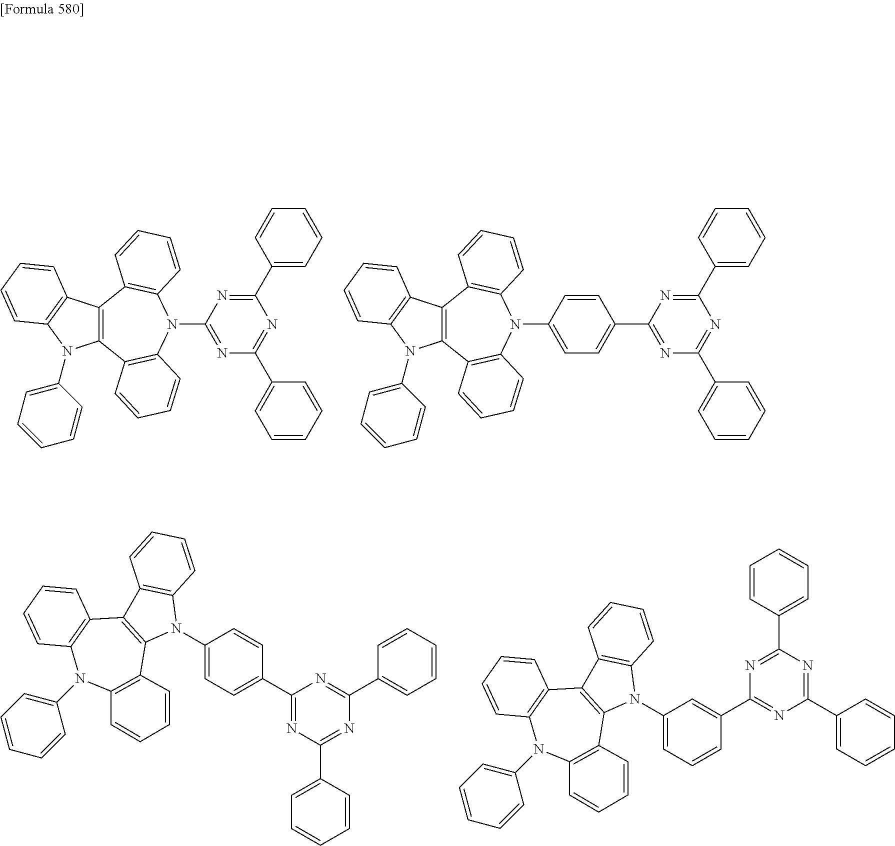

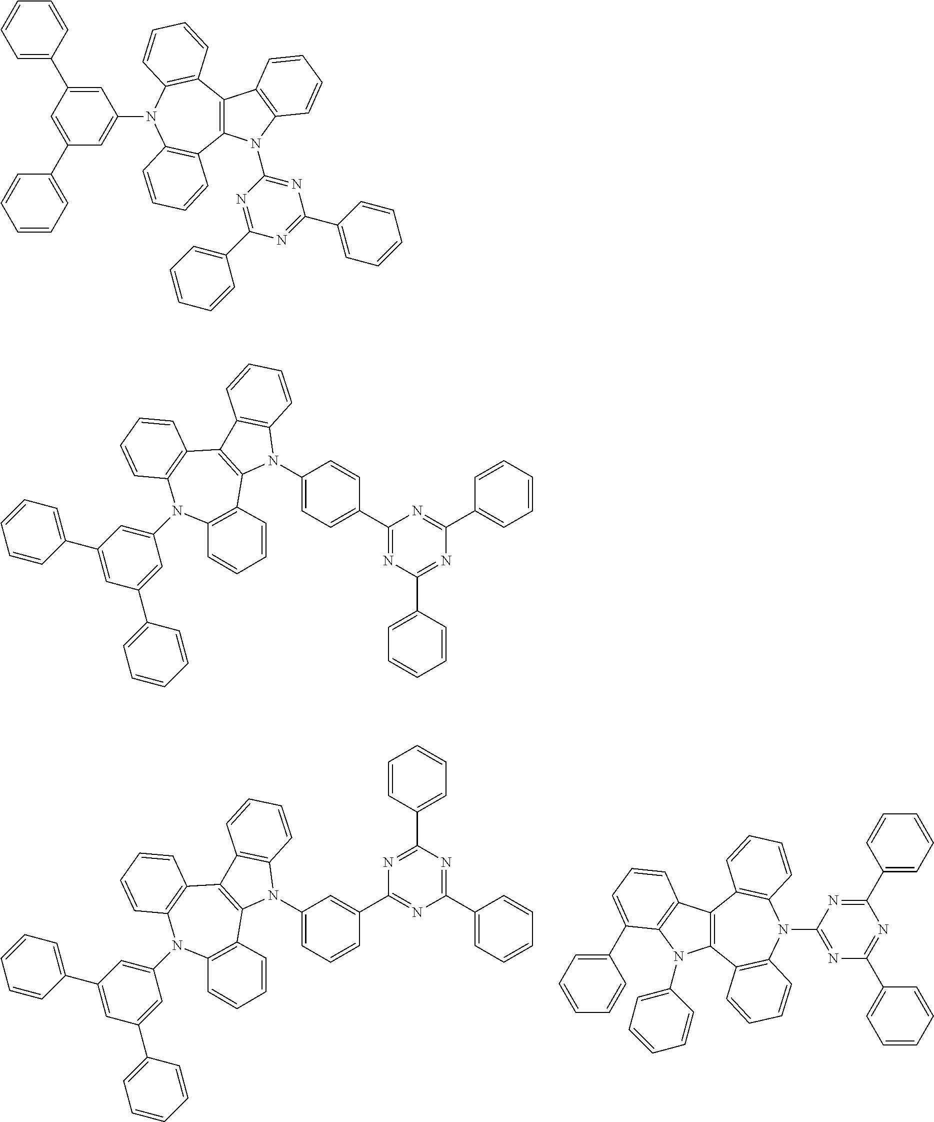

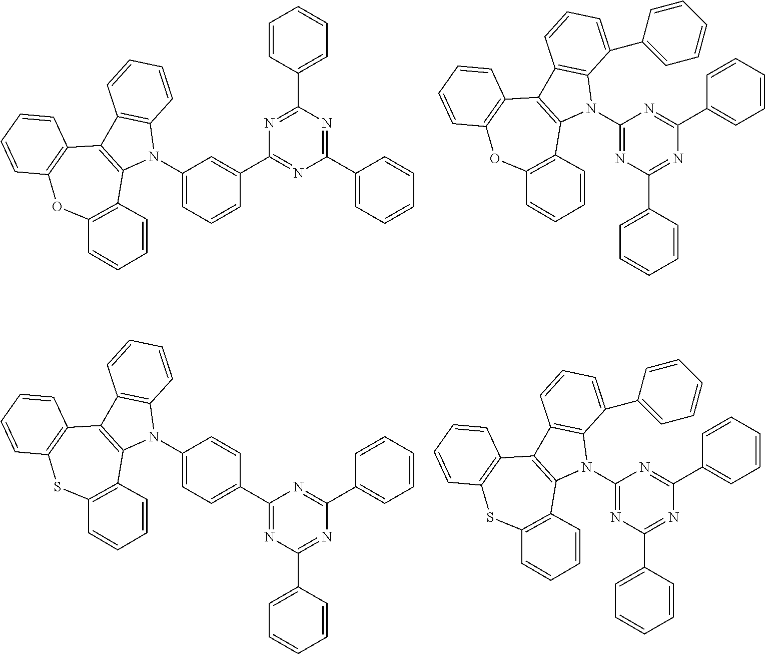

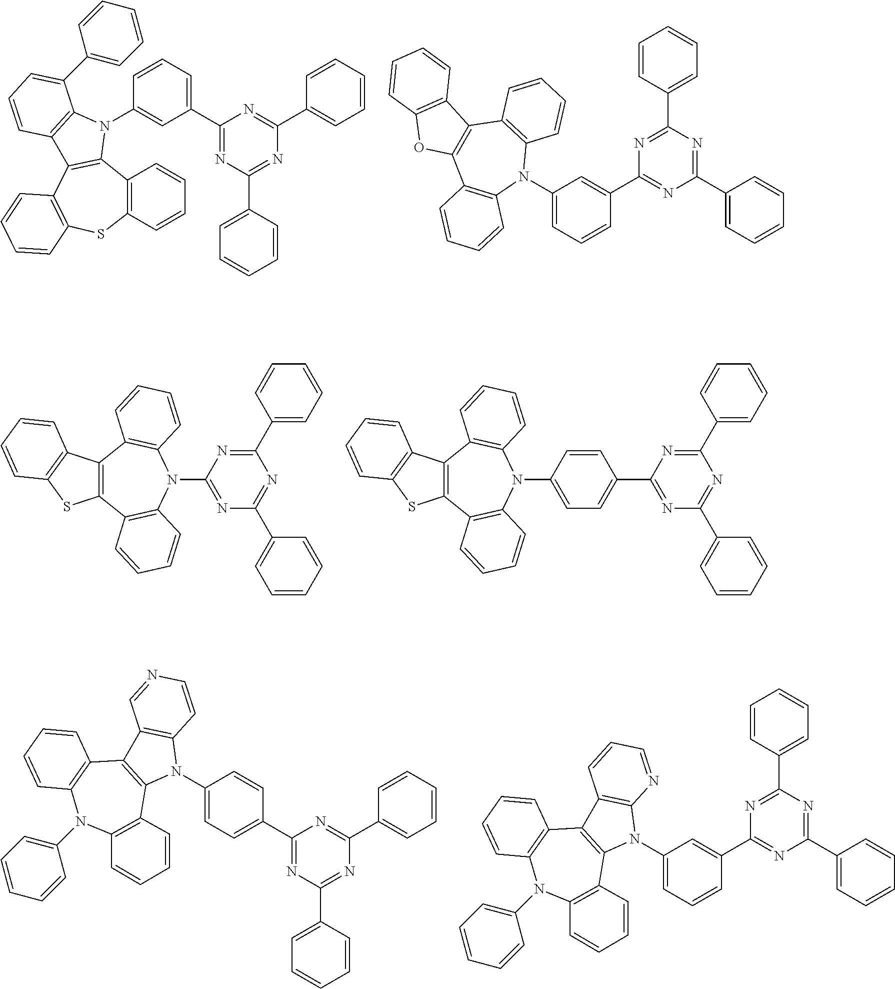

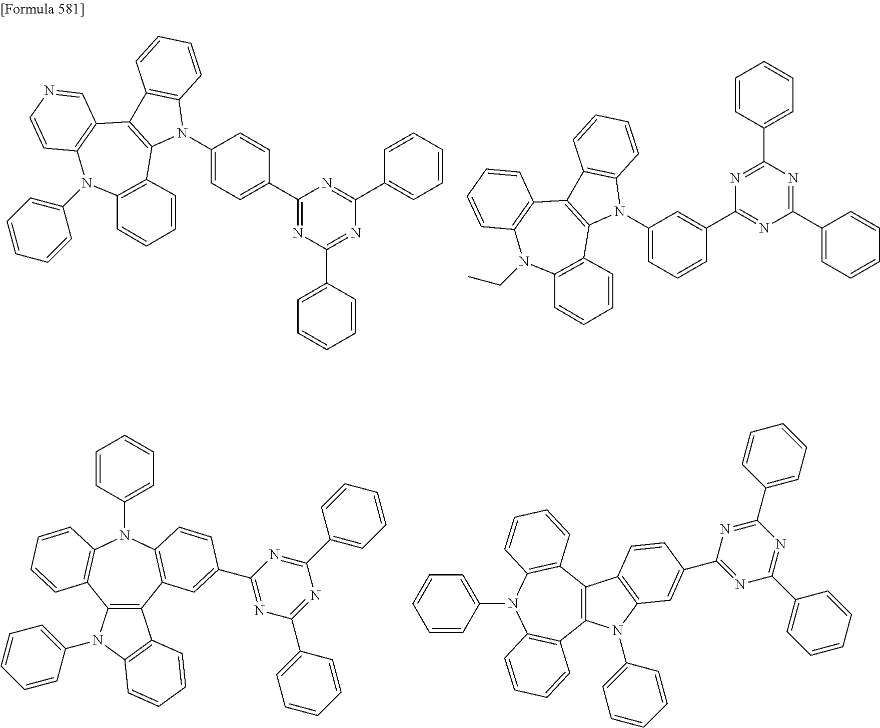

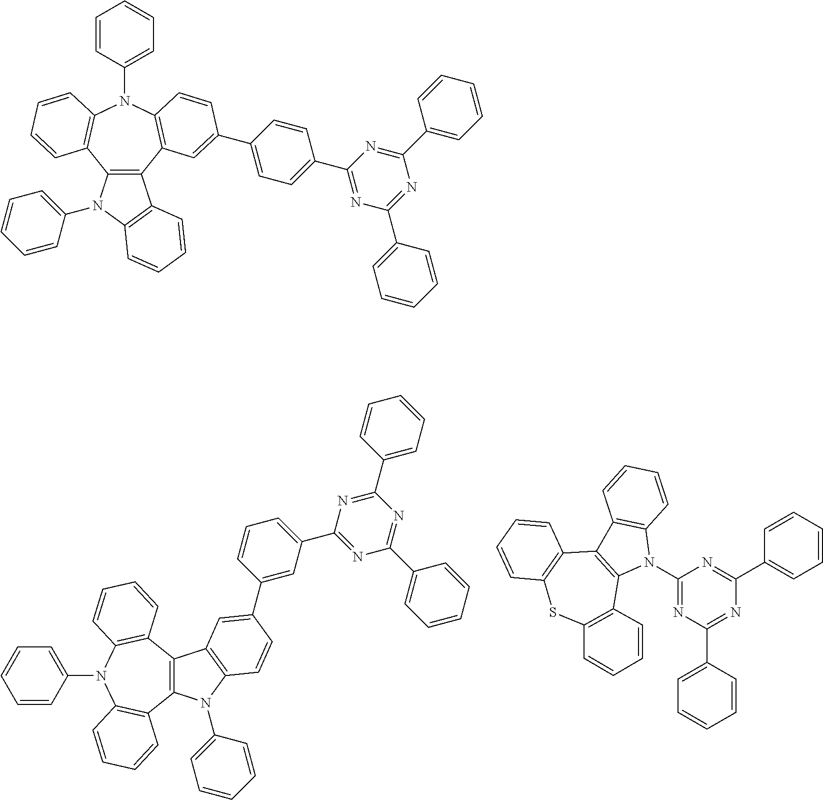

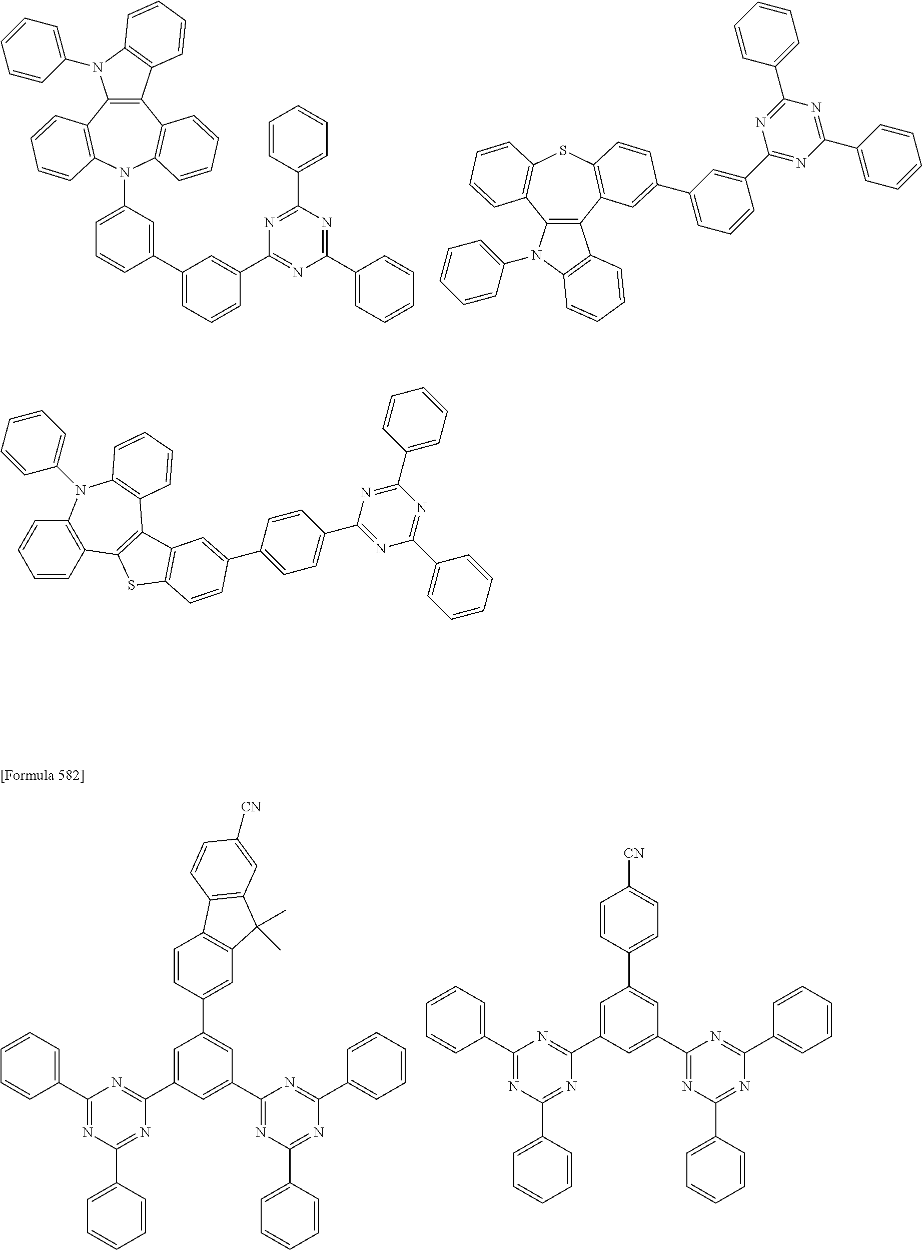

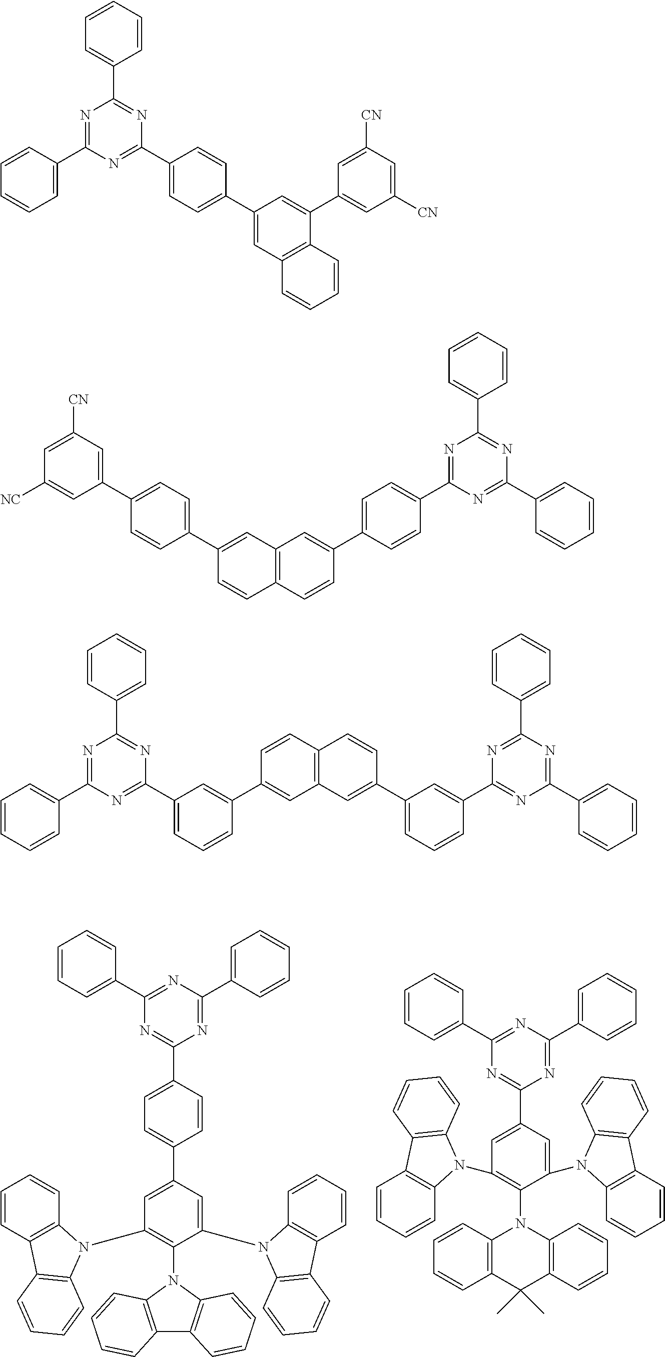

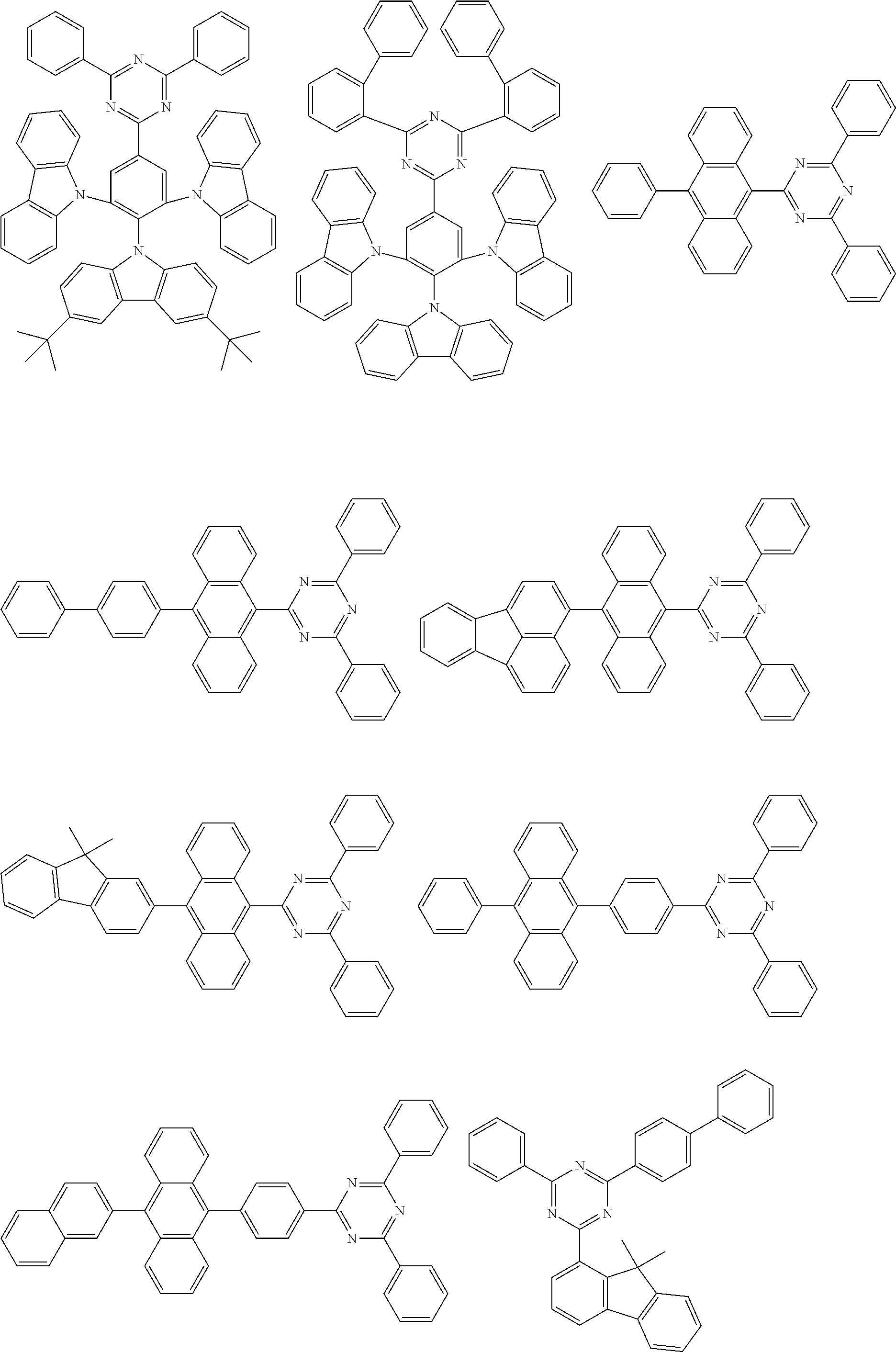

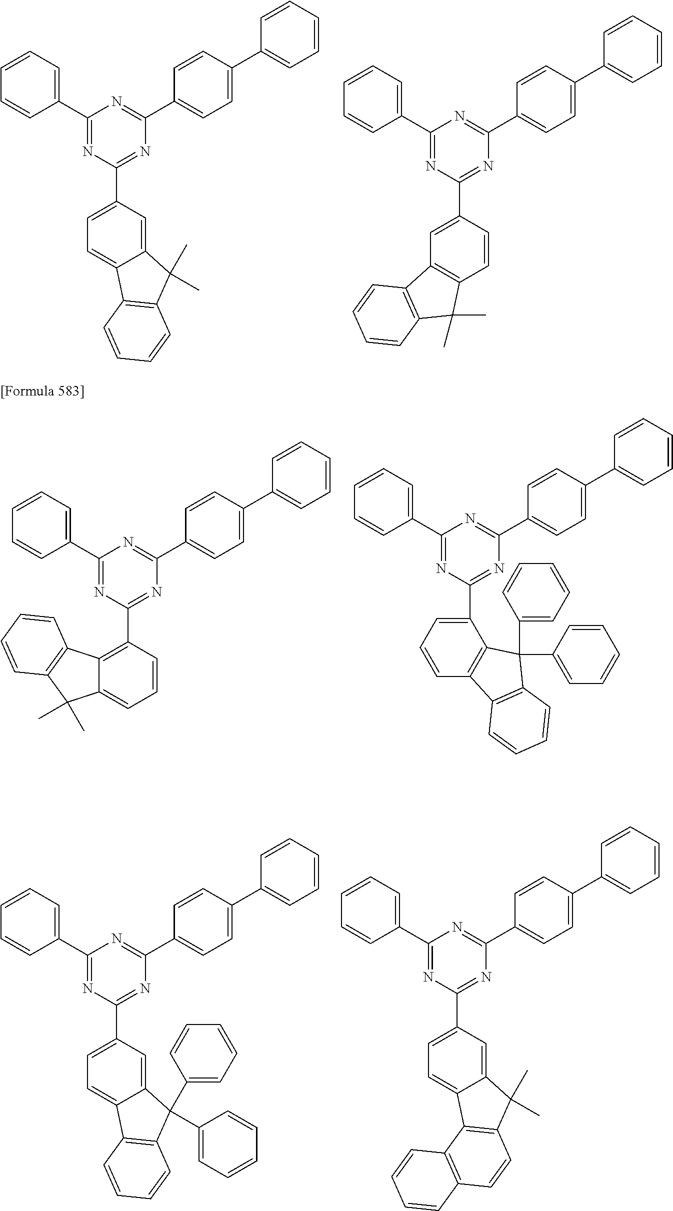

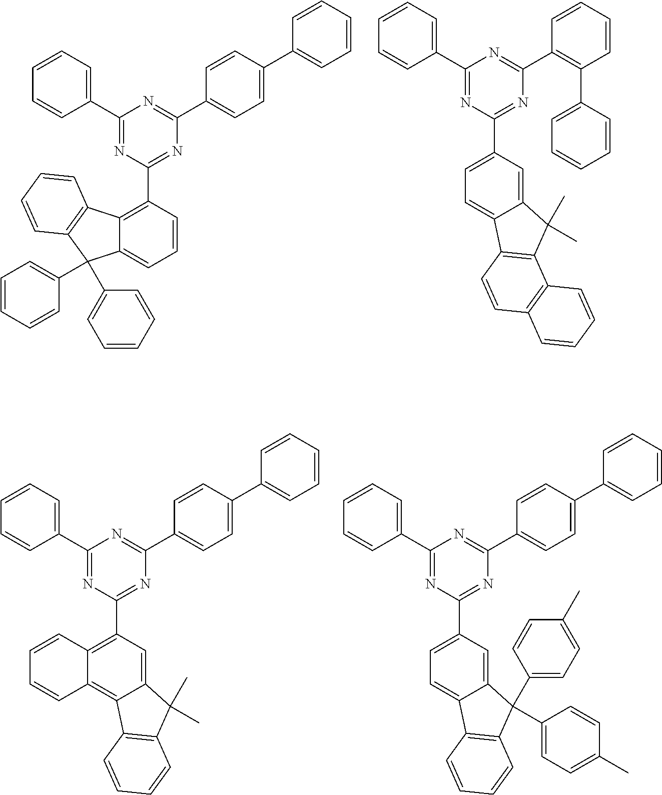

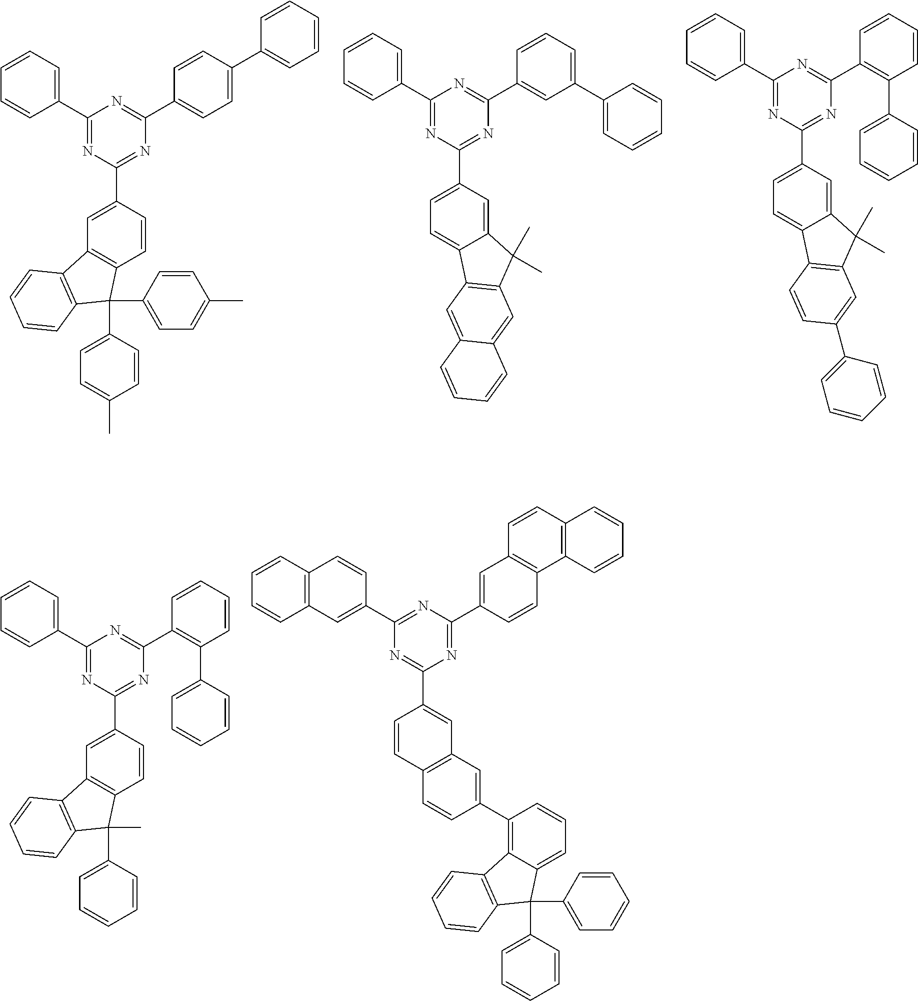

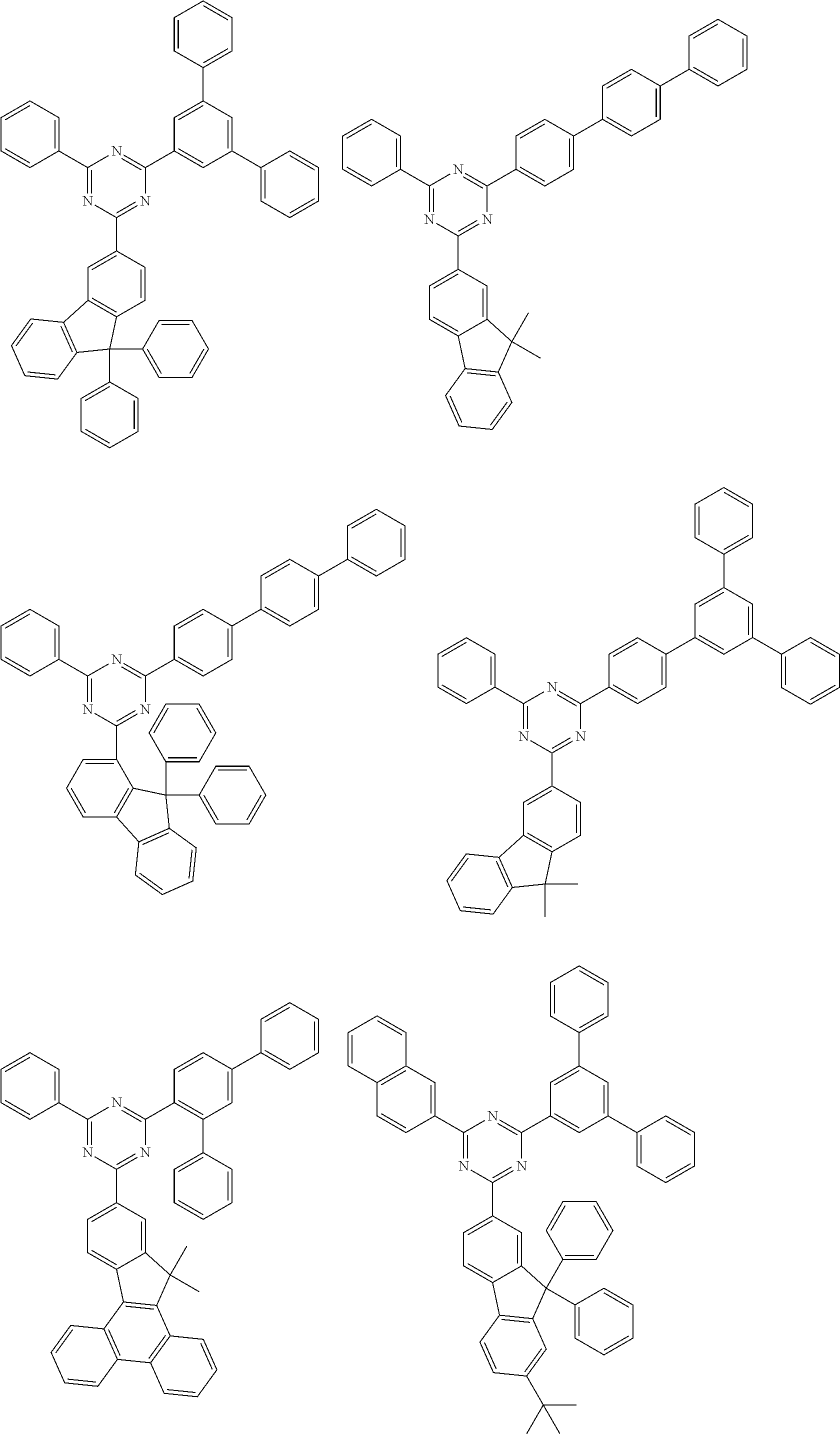

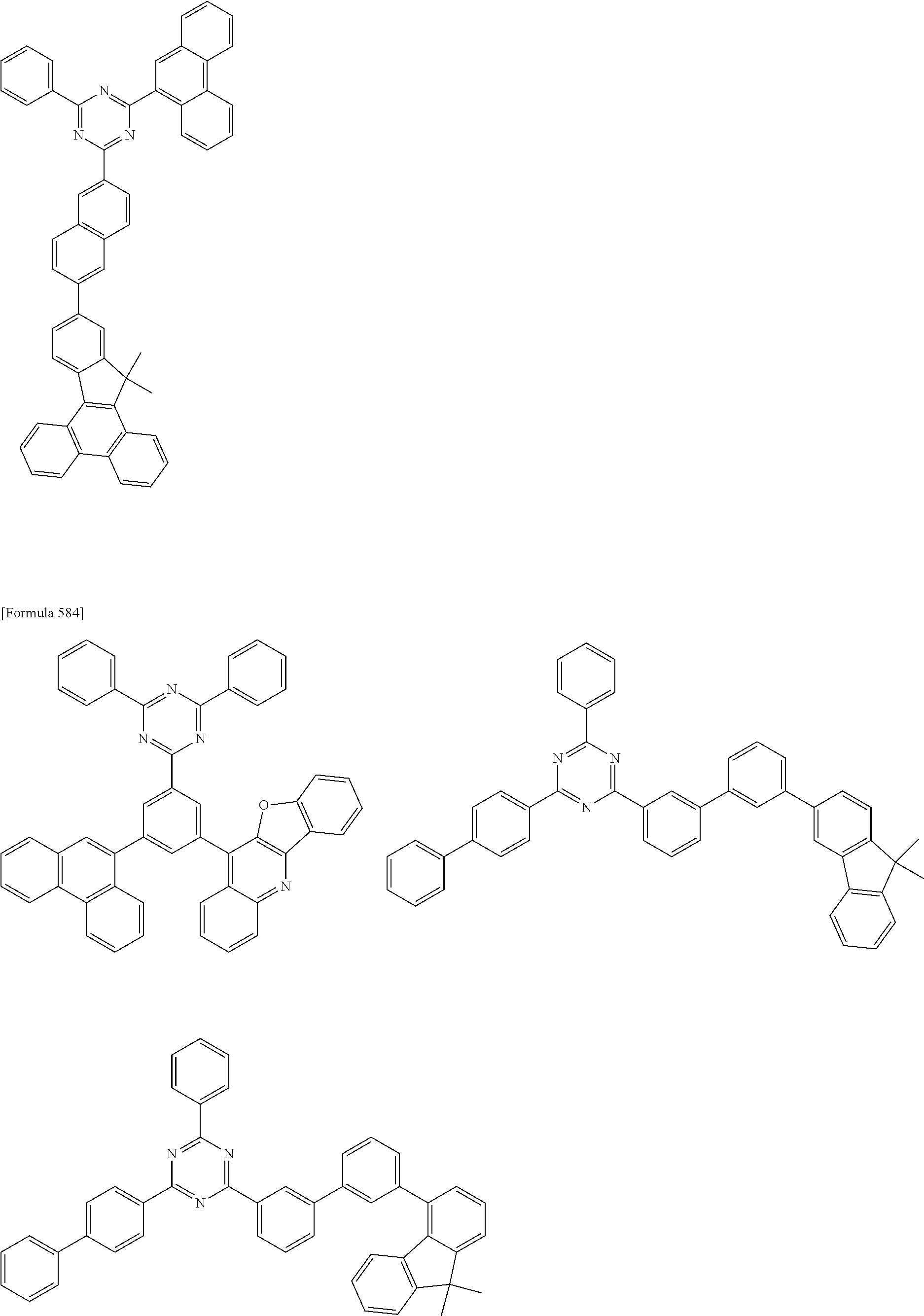 [SMILES: CC1(C)c2cc(-c3ccc4cc(-c5nc(-c6ccccc6)nc(-c6cc7ccccc7c7ccccc67)n5)ccc4c3)ccc2-c2c1c1ccccc1c1ccccc21 ; c1ccc(-c2nc(-c3ccccc3)nc(-c3cc(-c4cc5ccccc5c5ccccc45)cc(-c4c5ccccc5nc5c4oc4ccccc45)c3)n2)cc1 ; CC1(C)c2ccccc2-c2cc(-c3cccc(-c4cccc(-c5nc(-c6ccccc6)nc(-c6ccc(-c7ccccc7)cc6)n5)c4)c3)ccc21 ; CC1(C)c2ccccc2-c2c(-c3cccc(-c4cccc(-c5nc(-c6ccccc6)nc(-c6ccc(-c7ccccc7)cc6)n5)c4)c3)cccc21]

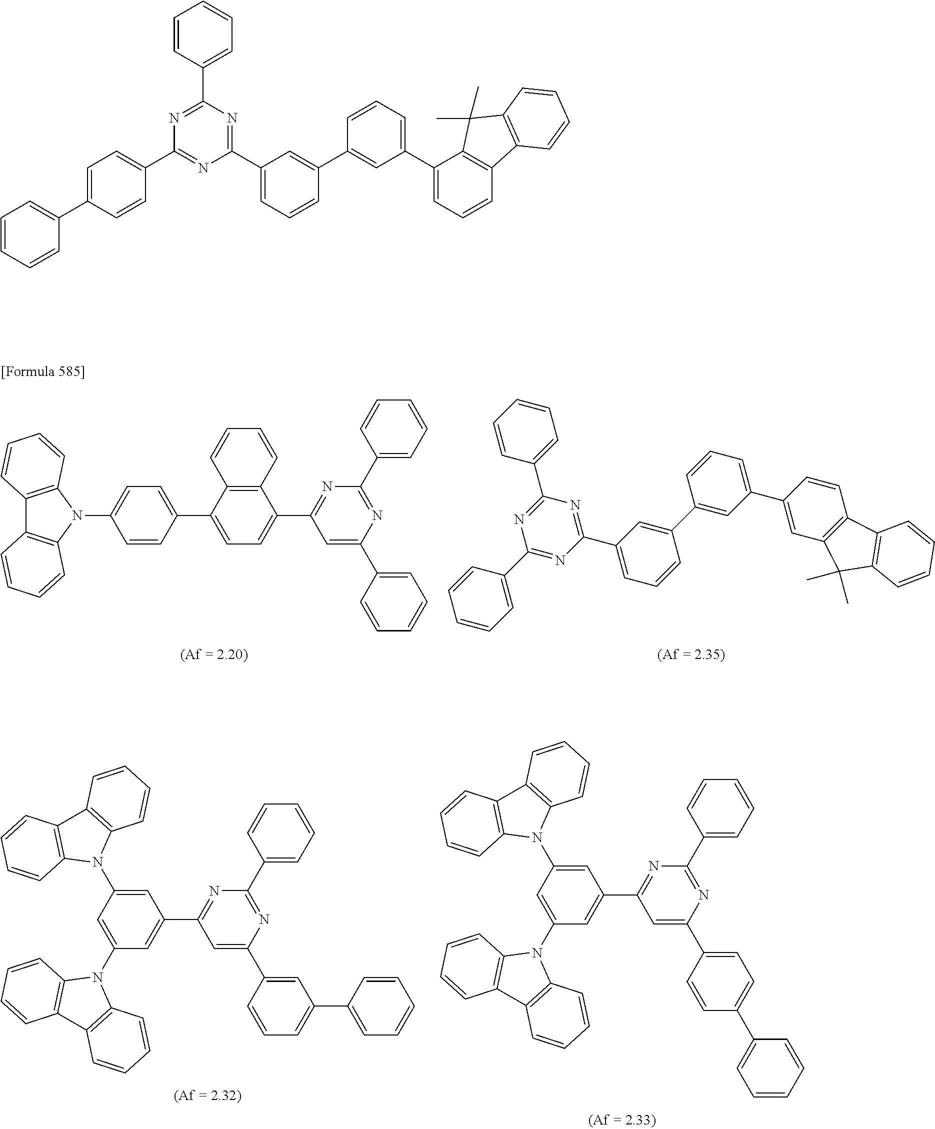

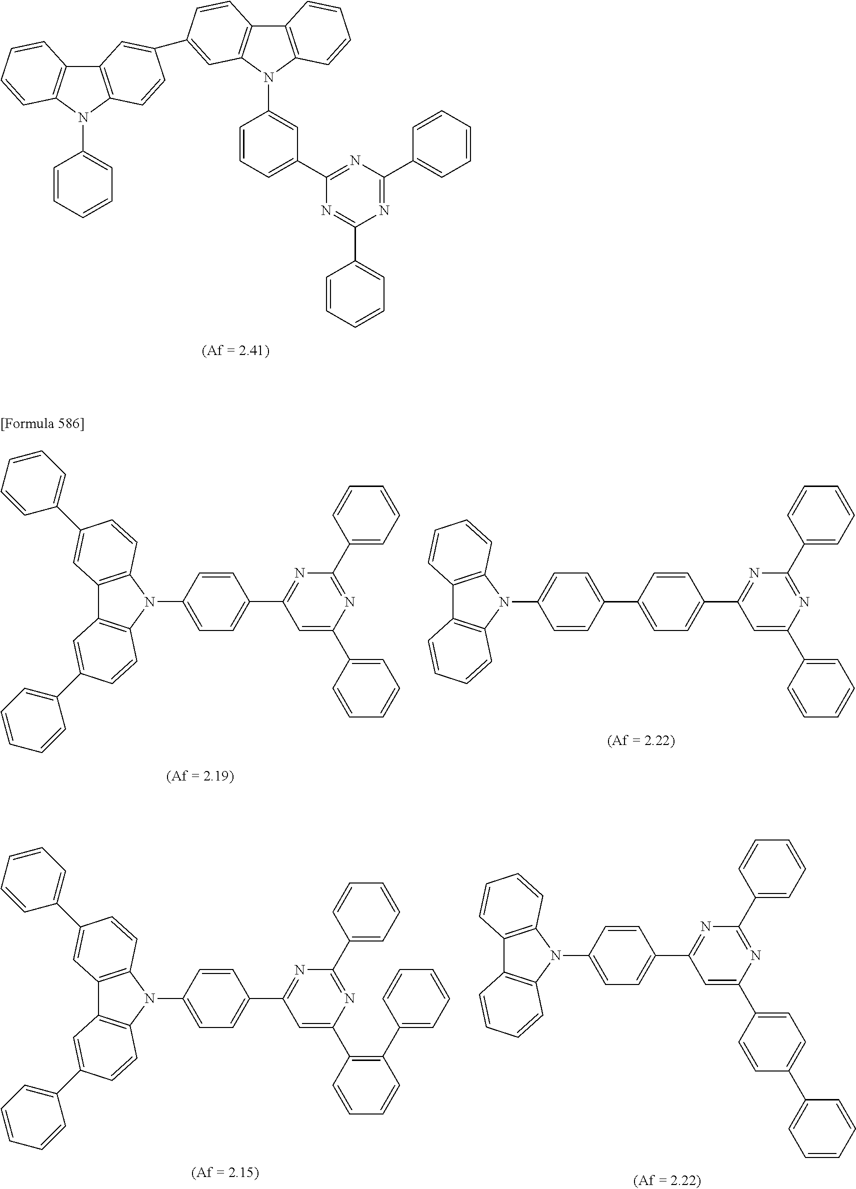

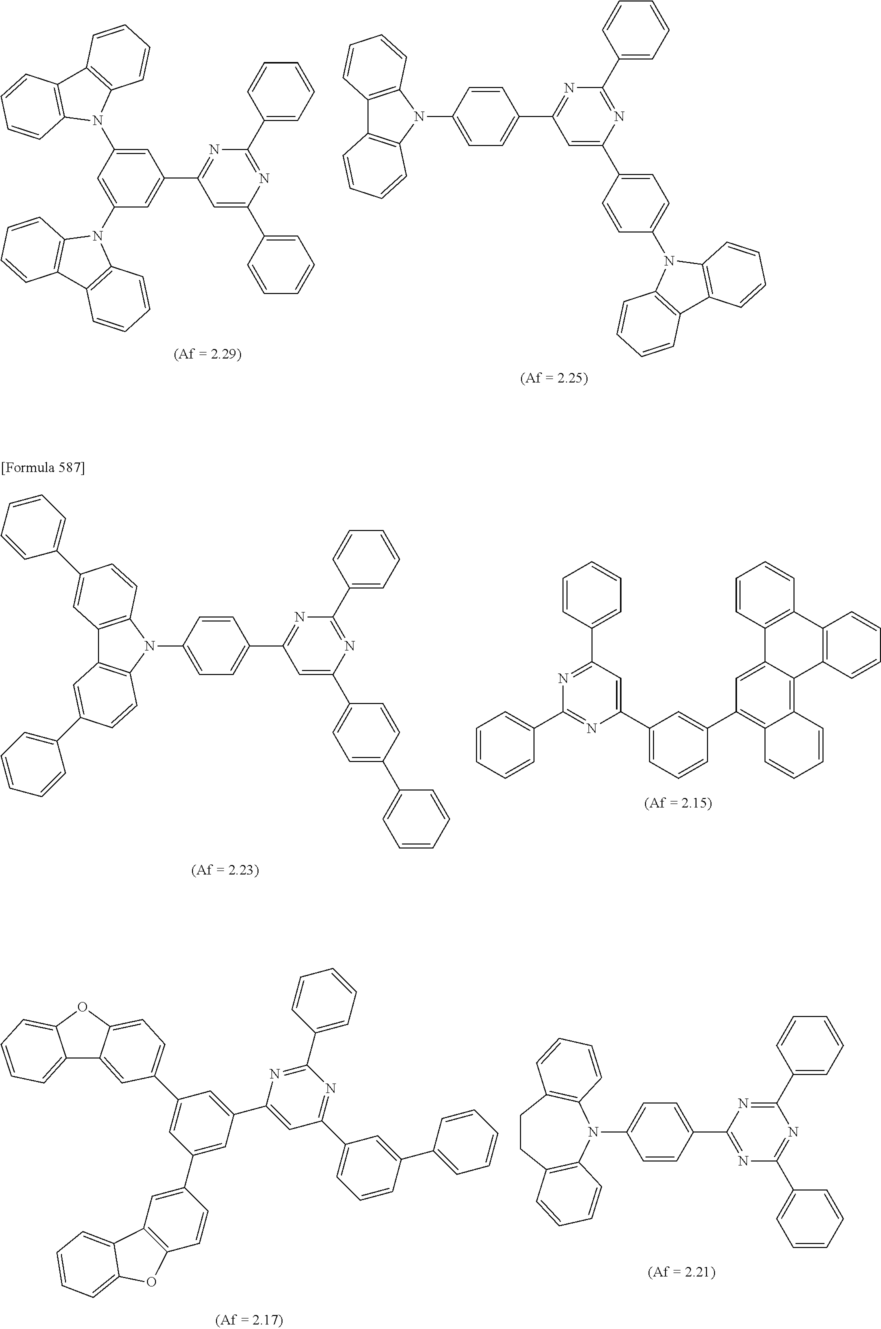

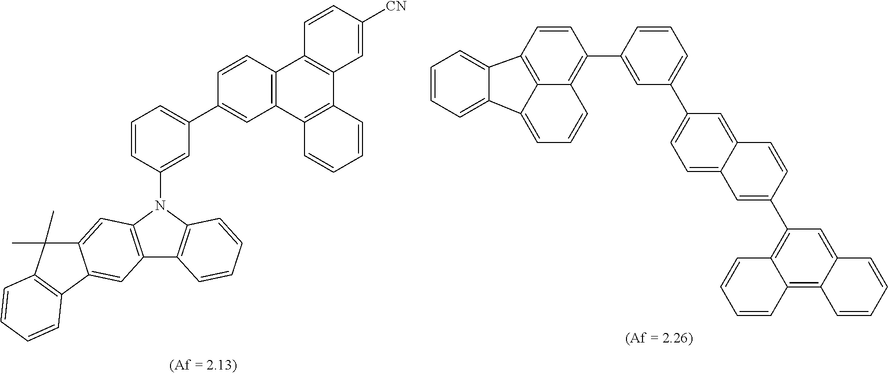

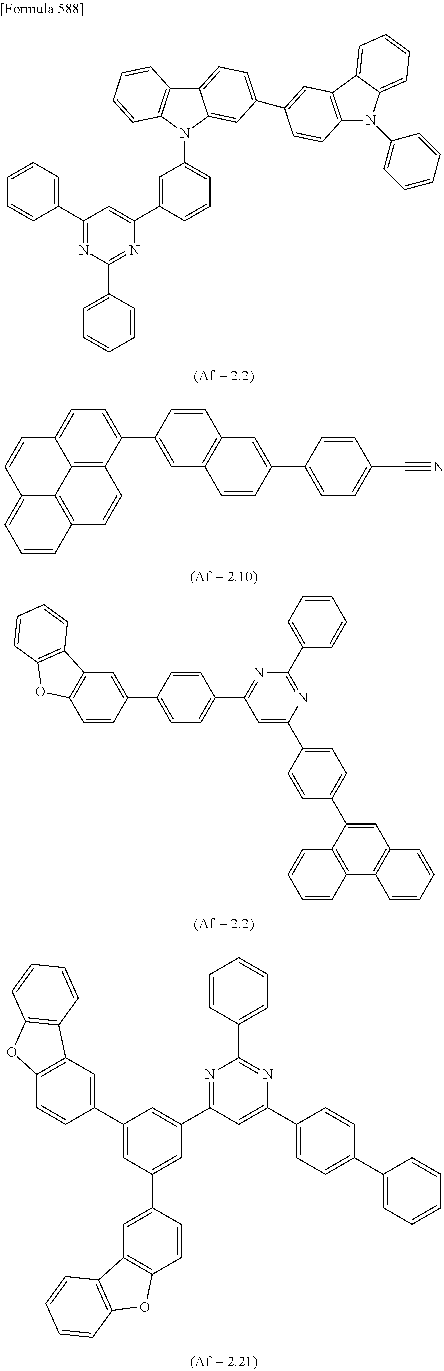 [SMILES: c1ccc(-c2cc(-c3cccc(-n4c5ccccc5c5ccc(-c6ccc7c(c6)c6ccccc6n7-c6ccccc6)cc54)c3)nc(-c3ccccc3)n2)cc1 ; N#Cc1ccc(-c2ccc3cc(-c4ccc5ccc6cccc7ccc4c5c67)ccc3c2)cc1 ; c1ccc(-c2nc(-c3ccc(-c4ccc5oc6ccccc6c5c4)cc3)cc(-c3ccc(-c4cc5ccccc5c5ccccc45)cc3)n2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

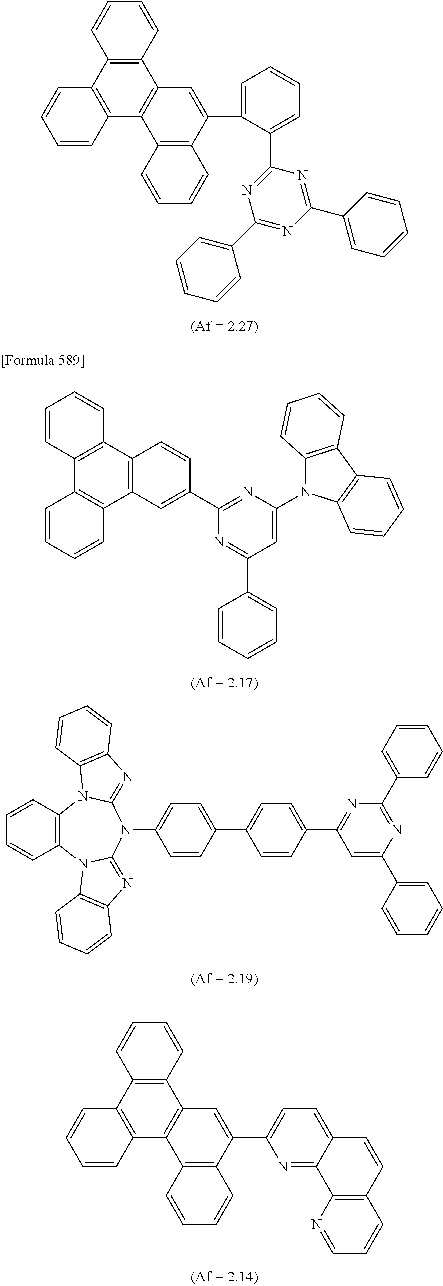 [SMILES: c1ccc(-c2nc(-c3ccccc3)nc(-c3ccccc3-c3cc4c5ccccc5c5ccccc5c4c4ccccc34)n2)cc1 ; c1ccc(-c2cc(-n3c4ccccc4c4ccccc43)nc(-c3ccc4c5ccccc5c5ccccc5c4c3)n2)cc1 ; c1ccc(-c2cc(-c3ccc(-c4ccc(-n5c6nc7ccccc7n6c6ccccc6n6c7ccccc7nc56)cc4)cc3)nc(-c3ccccc3)n2)cc1 ; c1cnc2c(c1)ccc1ccc(-c3cc4c5ccccc5c5ccccc5c4c4ccccc34)nc12]

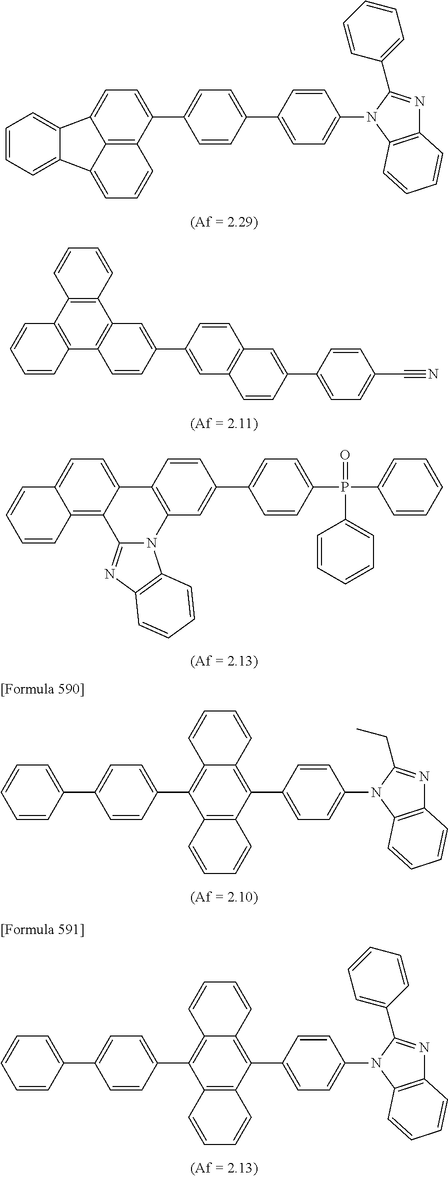 [SMILES: c1ccc(-c2nc3ccccc3n2-c2ccc(-c3ccc(-c4ccc5c6c(cccc46)-c4ccccc4-5)cc3)cc2)cc1 ; N#Cc1ccc(-c2ccc3cc(-c4ccc5c6ccccc6c6ccccc6c5c4)ccc3c2)cc1 ; O=P(c1ccccc1)(c1ccccc1)c1ccc(-c2ccc3c4ccc5ccccc5c4c4nc5ccccc5n4c3c2)cc1 ; CCc1nc2ccccc2n1-c1ccc(-c2c3ccccc3c(-c3ccc(-c4ccccc4)cc3)c3ccccc23)cc1 ; c1ccc(-c2ccc(-c3c4ccccc4c(-c4ccc(-n5c(-c6ccccc6)nc6ccccc65)cc4)c4ccccc34)cc2)cc1]

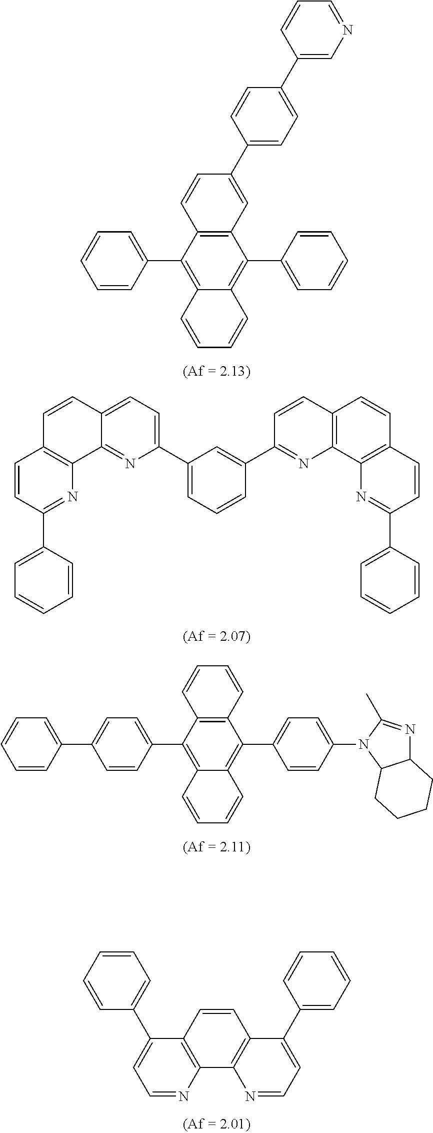 [SMILES: c1ccc(-c2c3ccccc3c(-c3ccccc3)c3cc(-c4ccc(-c5cccnc5)cc4)ccc23)cc1 ; c1ccc(-c2ccc3ccc4ccc(-c5cccc(-c6ccc7ccc8ccc(-c9ccccc9)nc8c7n6)c5)nc4c3n2)cc1 ; CC1=NC2CCCCC2N1c1ccc(-c2c3ccccc3c(-c3ccc(-c4ccccc4)cc3)c3ccccc23)cc1 ; c1ccc(-c2ccnc3c2ccc2c(-c4ccccc4)ccnc23)cc1]

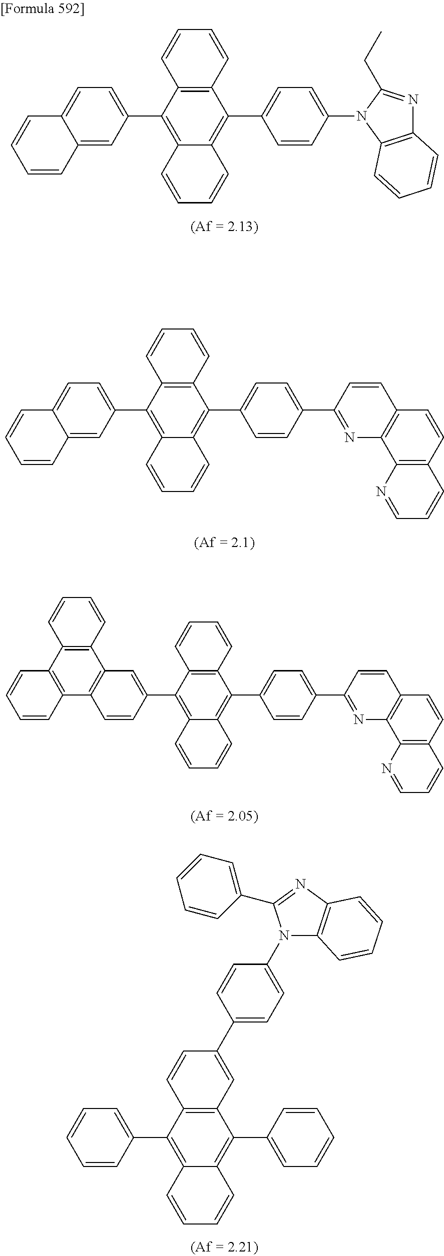 [SMILES: CCc1nc2ccccc2n1-c1ccc(-c2c3ccccc3c(-c3ccc4ccccc4c3)c3ccccc23)cc1 ; c1ccc2cc(-c3c4ccccc4c(-c4ccc(-c5ccc6ccc7cccnc7c6n5)cc4)c4ccccc34)ccc2c1 ; c1cnc2c(c1)ccc1ccc(-c3ccc(-c4c5ccccc5c(-c5ccc6c7ccccc7c7ccccc7c6c5)c5ccccc45)cc3)nc12 ; c1ccc(-c2c3ccccc3c(-c3ccccc3)c3cc(-c4ccc(-n5c(-c6ccccc6)nc6ccccc65)cc4)ccc23)cc1]

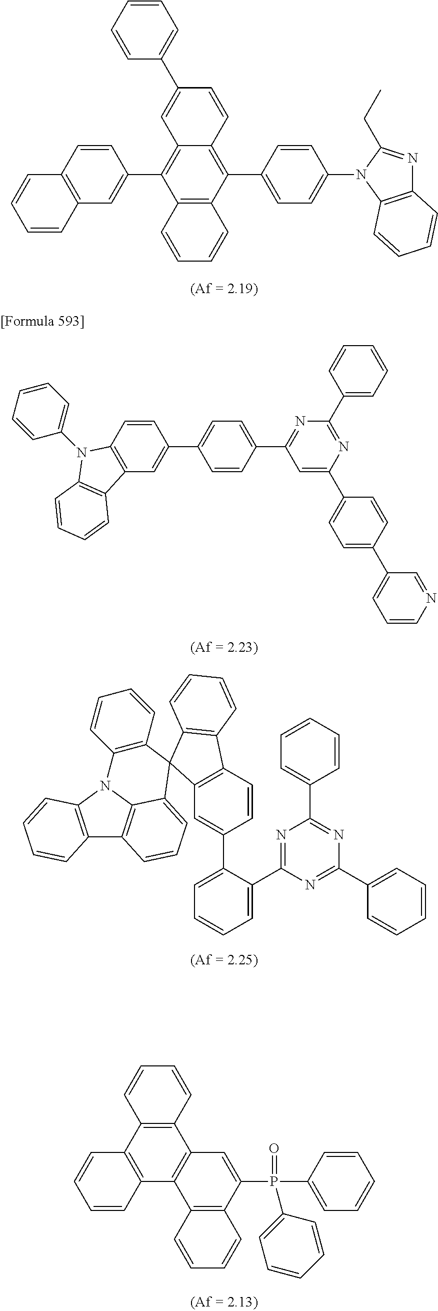 [SMILES: CCc1nc2ccccc2n1-c1ccc(-c2c3ccccc3c(-c3ccc4ccccc4c3)c3cc(-c4ccccc4)ccc23)cc1 ; c1ccc(-c2nc(-c3ccc(-c4cccnc4)cc3)cc(-c3ccc(-c4ccc5c(c4)c4ccccc4n5-c4ccccc4)cc3)n2)cc1 ; c1ccc(-c2nc(-c3ccccc3)nc(-c3ccccc3-c3ccc4c(c3)C3(c5ccccc5-4)c4ccccc4-n4c5ccccc5c5cccc3c54)n2)cc1 ; O=P(c1ccccc1)(c1ccccc1)c1cc2c3ccccc3c3ccccc3c2c2ccccc12]

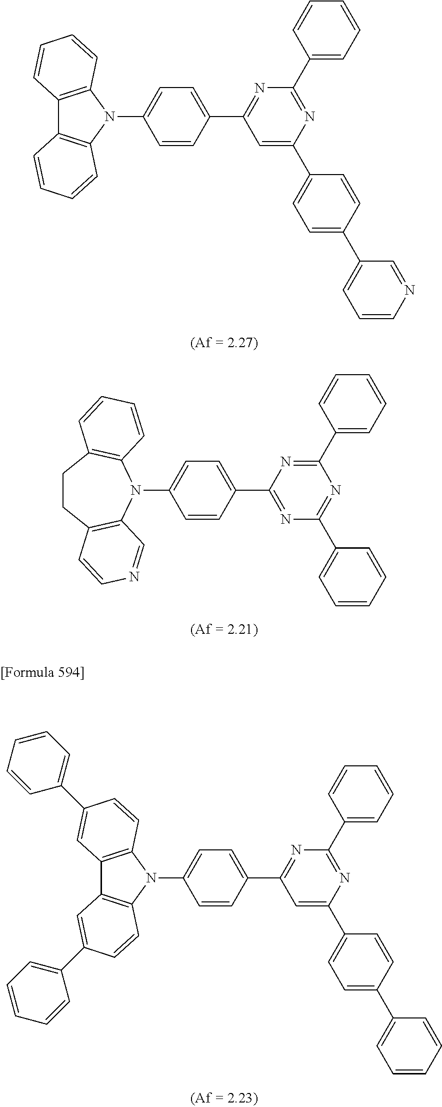 [SMILES: c1ccc(-c2nc(-c3ccc(-c4cccnc4)cc3)cc(-c3ccc(-n4c5ccccc5c5ccccc54)cc3)n2)cc1 ; c1ccc(-c2nc(-c3ccccc3)nc(-c3ccc(N4c5ccccc5CCc5ccncc54)cc3)n2)cc1 ; c1ccc(-c2ccc(-c3cc(-c4ccc(-n5c6ccc(-c7ccccc7)cc6c6cc(-c7ccccc7)ccc65)cc4)nc(-c4ccccc4)n3)cc2)cc1]

In an exemplary arrangement of the organic EL device of each exemplary embodiment, the electron transporting layer is provided between the first emitting region and the cathode. In the organic EL device of each exemplary embodiment, the electron transporting layer that may be included in the electron transporting zone is not limited to the electron transporting layer containing the nitrogen-containing compound.

The electron transporting layer is a layer containing a highly electron-transportable substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, for instance, a metal complex such as Alq, tris(4-methyl-8-quinolinolato)aluminum (abbreviation: Almq₃), bis(10-hydroxybenzo[h]quinolinato) beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl) stilbene (abbreviation: BzOs) is also usable. In an exemplary arrangement of the organic EL device of each exemplary embodiment, a nitrogen-containing compound (preferably benzimidazole compound) is suitably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be a single layer or a laminate of two or more layers formed of the above substance.

Further, a high polymer compound is usable for the electron transporting layer. For instance, poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are usable.

### Hole Blocking Layer

In an exemplary arrangement of the organic EL device of each exemplary embodiment, the electron transporting zone may include a hole blocking layer. An electron transporting layer disposed to a side close to the cathode of the emitting layer is occasionally referred to as a hole blocking layer.

The hole blocking layer is preferably a layer transporting electrons and blocking holes from reaching a layer close to the cathode (e.g., electron transporting layer and electron injecting layer) beyond the hole blocking layer. The compound contained in the hole blocking layer is exemplified by a compound used in a known hole blocking layer. The compound contained in the hole blocking layer, which is similar to a compound usable in the electron transporting layer, is preferably at least one compound selected from the group consisting of a metal complex, a heteroaromatic compound, and a high polymer compound. The compound contained in the hole blocking layer may be a nitrogen-containing compound, for instance, at least one compound selected from the group consisting of an imidazole derivative, benzimidazole derivative, azine derivative, carbazole derivative, and phenanthroline derivative.

In order to prevent excitation energy from leaking out from the emitting layer toward neighboring layer(s), the hole blocking layer is also preferably a layer blocking excitons generated in the emitting layer from being transferred to a layer(s) closer to the cathode (e.g., the electron transporting layer and the electron injecting layer) beyond the hole blocking layer.

### Electron Injecting Layer

In an exemplary arrangement of the organic EL device of each exemplary embodiment, the electron transporting zone may include an electron injecting layer.

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is also usable. Further, the organic compound such as tetrathiafulvalene (abbreviation: TTF) is also usable.

### Schematic Arrangements of Organic EL Devices

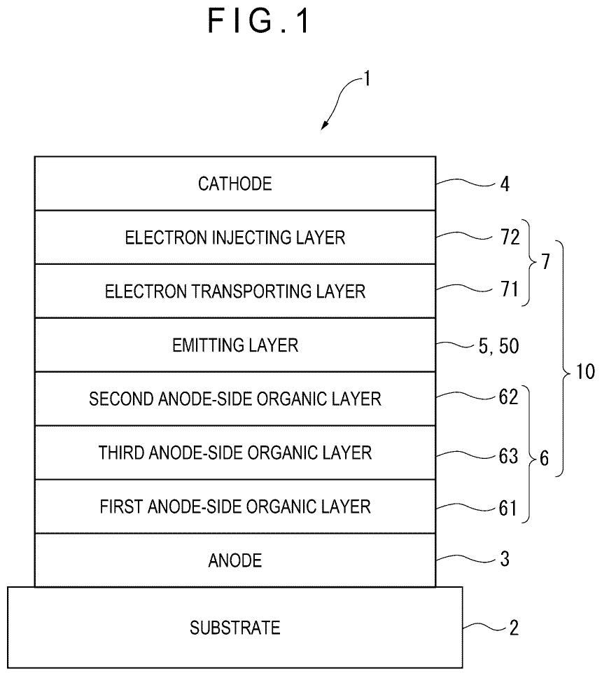

FIGS. 1 to 6 schematically depict exemplary arrangements of organic EL devices according to the first exemplary embodiment, the second exemplary embodiment, and the third exemplary embodiment.

An organic EL device 1 depicted in FIG. 1 includes a substrate 2, an anode 3, a cathode 4, and an organic layer 10 provided between the anode 3 and the cathode 4. The organic layer 10 includes a first hole transporting zone 6, a first emitting region 5, and a first electron transporting zone 7 in this order from a side close to the anode 3. The first hole transporting zone 6 includes a first anode-side organic layer 61, a third anode-side organic layer 63, and a second anode-side organic layer 62 in this order from a side close to the anode 3. The first emitting region 5 includes a single emitting layer 50. The first electron transporting zone 7 includes an electron transporting layer 71 and an electron injecting layer 72 in this order from a side close to the first emitting region 5.

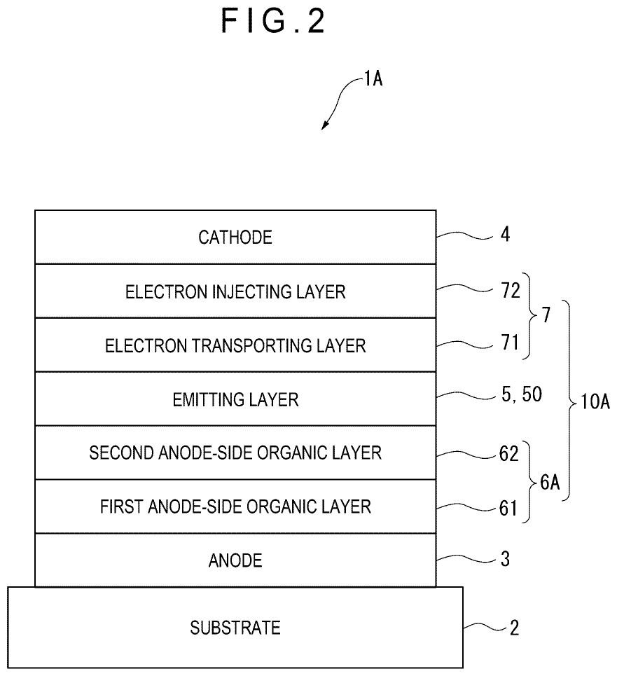

An organic EL device 1A depicted in FIG. 2 is different from the organic EL device 1 in that an organic layer 10A includes a first hole transporting zone 6A, and the rest of components and arrangement of the organic EL device 1A are the same as those of the organic EL device 1. The first hole transporting zone 6A includes the first anode-side organic layer 61 and the second anode-side organic layer 62 in this order from a side close to the anode 3, but does not include the third anode-side organic layer.

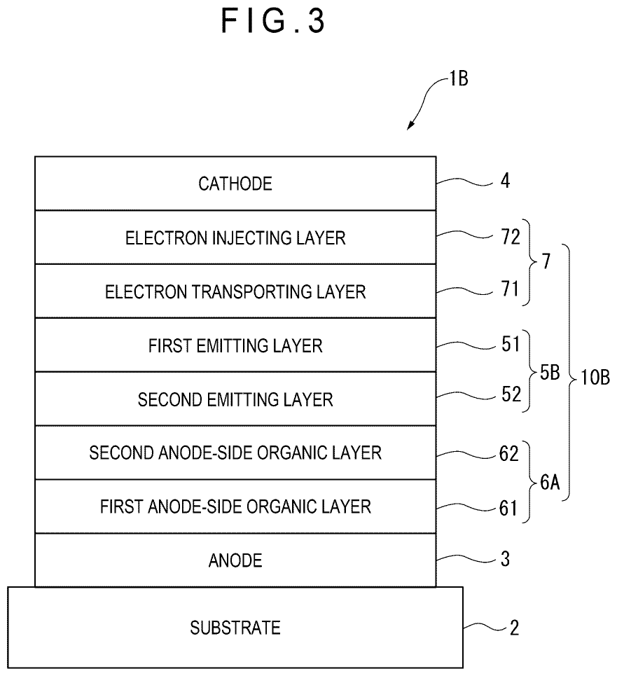

An organic EL device 1B depicted in FIG. 3 is different from the organic EL device 1A in that an organic layer 10B includes a first emitting region 5B, and the rest of components and arrangement of the organic EL device 1B are the same as those of the organic EL device 1A. The first emitting region 5B includes the second first emitting layer 52 and the first emitting layer 51 in this order from a side close to the anode 3.

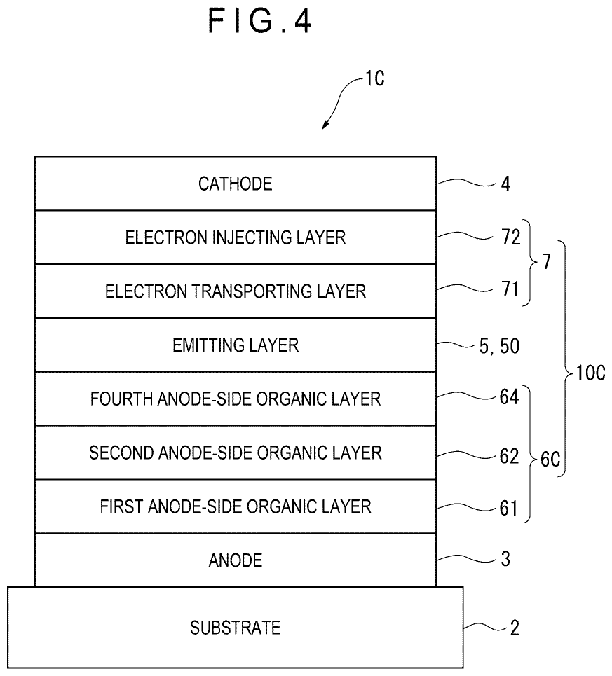

An organic EL device 1C depicted in FIG. 4 is different from the organic EL device 1 in that an organic layer 10C includes a first hole transporting zone 6C, and the rest of components and arrangement of the organic EL device 1C are the same as those of the organic EL device 1. The first hole transporting zone 6C includes the first anode-side organic layer 61, the second anode-side organic layer 62, and a fourth anode-side organic layer 64 in this order from a side close to the anode 3, but does not include the third anode-side organic layer.

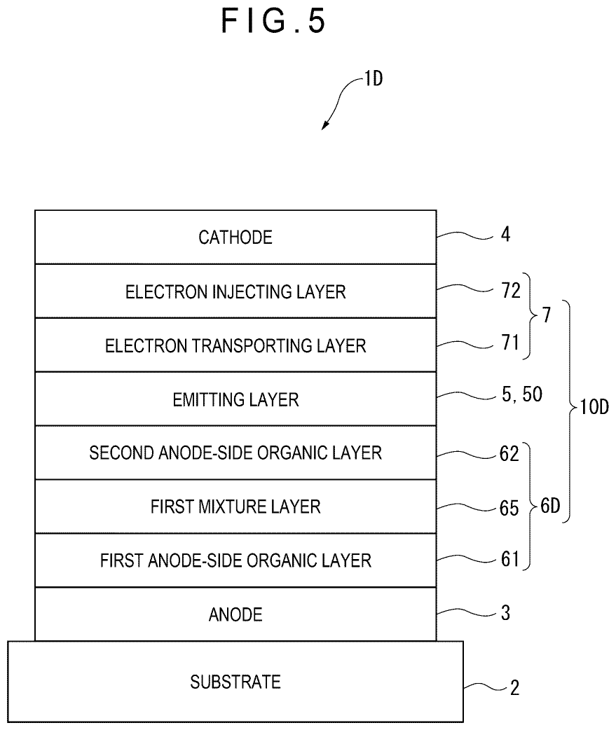

An organic EL device 1D depicted in FIG. 5 is different from the organic EL device 1A depicted in FIG. 2 in that an organic layer 10D includes a hole transporting zone 6D, and the rest of components and arrangement of the organic EL device 1D are the same as those of the organic EL device 1A. The hole transporting zone 6D includes a first mixture layer 65 between the first anode-side organic layer 61 and the second anode-side organic layer 62. In the hole transporting zone 6D, the first anode-side organic layer 61, the first mixture layer 65, and the second anode-side organic layer 62 are disposed in this order from a side close to the anode 3.

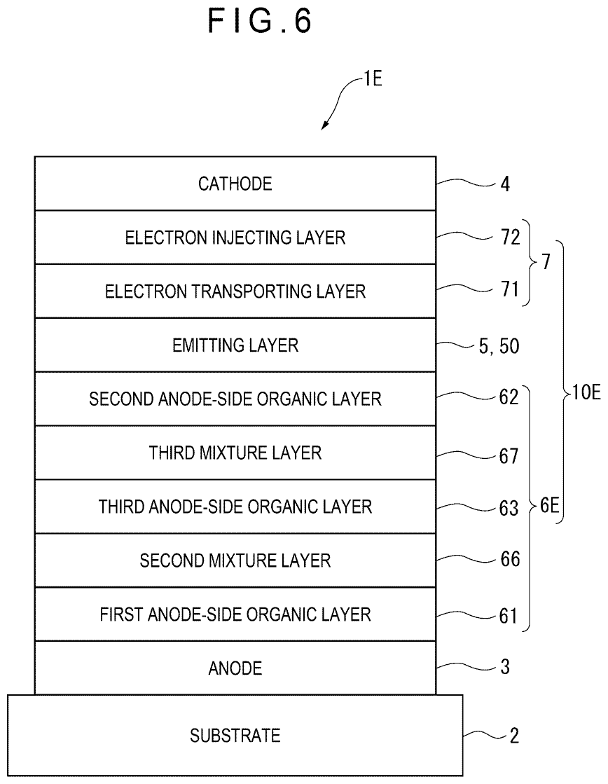

An organic EL device 1E depicted in FIG. 6 is different from the organic EL device 1 depicted in FIG. 1 in that an organic layer 10E includes a hole transporting zone 6E, and the rest of components and arrangement of the organic EL device 1E are the same as those of the organic EL device 1. The hole transporting zone 6E includes: a second mixture layer 66 between the first anode-side organic layer 61 and the third anode-side organic layer 63; and a third mixture layer 67 between the third anode-side organic layer 63 and the second anode-side organic layer 62. In the hole transporting zone 6E, the first anode-side organic layer 61, the second mixture layer 66, the third anode-side organic layer 63, the third mixture layer 67, and the second anode-side organic layer 62 are disposed in this order from a side close to the anode 3.

The invention is not limited to the exemplary arrangements of the organic EL devices depicted in FIGS. 1 to 6. An organic EL device with another exemplary arrangement is exemplified by an organic EL device including a first emitting region 5B in place of the first emitting region 5 of each of the organic EL devices depicted in FIGS. 1, 2, 4, 5, and 6.

Moreover, an organic EL device with still another exemplary arrangement is exemplified by an organic EL device in which the order of the emitting layers layered in the first emitting region 5 may be changed: the first emitting layer and the second emitting layer are layered in the first emitting region 5B in this order from a side close to the anode. Regardless of the order of the first emitting layer and the second emitting layer layered, the effect obtained by layering the first and second emitting layers in the first emitting region can be expected by selecting a combination of materials satisfying the relationship of the numerical formula (Numerical Formula 1).

Further, an organic EL device with a further exemplary arrangement may include, for instance, the first mixture layer between the first anode-side organic layer 61 and the second anode-side organic layer 62.

Furthermore, an organic EL device with a still further exemplary arrangement may include, for instance, the second mixture layer between the first anode-side organic layer 61 and the third anode-side organic layer 63 and the third mixture layer between the third anode-side organic layer 63 and the second anode-side organic layer 62.

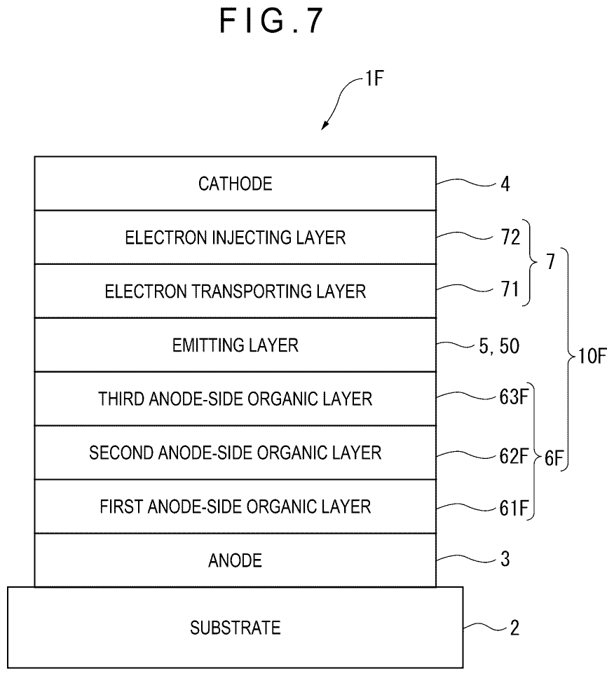

FIGS. 7 to 9 schematically depict exemplary arrangements of organic EL devices according to the fourth exemplary embodiment, the fifth exemplary embodiment, and the sixth exemplary embodiment.

An organic EL device 1F depicted in FIG. 7 includes the substrate 2, the anode 3, the cathode 4, and an organic layer 10F provided between the anode 3 and the cathode 4. The organic layer 10F includes a first hole transporting zone 6F, the first emitting region 5, and the first electron transporting zone 7 in this order from a side close to the anode 3. The first hole transporting zone 6F includes a first anode-side organic layer 61F, a second anode-side organic layer 62F, and a third anode-side organic layer 63F in this order from a side close to the anode 3. The first emitting region 5 includes a single emitting layer 50. The first electron transporting zone 7 includes an electron transporting layer 71 and an electron injecting layer 72 in this order from a side close to the first emitting region 5.

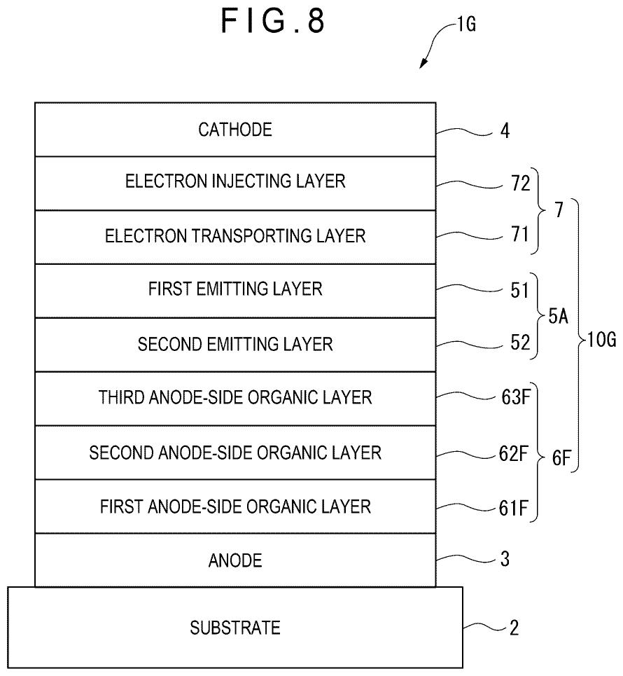

An organic EL device 1G depicted in FIG. 8 is different from the organic EL device 1F in that an organic layer 10G includes the first emitting region 5A, and the rest of components and arrangement of the organic EL device 1G are the same as those of the organic EL device 1F. The first emitting region 5A includes the second first emitting layer 52 and the first emitting layer 51 in this order from a side close to the anode 3.

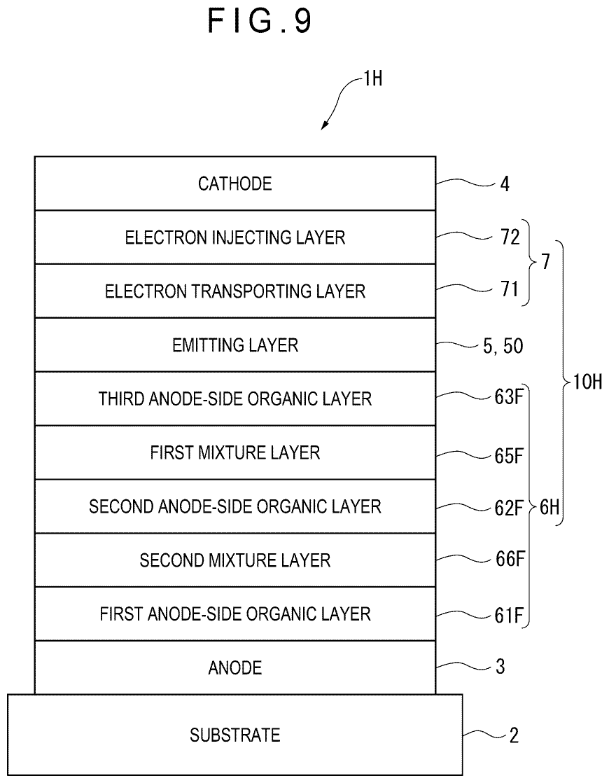

An organic EL device 1H depicted in FIG. 9 is different from the organic EL device 1F depicted in FIG. 7 in that an organic layer 10H includes a hole transporting zone 6H, and the rest of components and arrangement of the organic EL device 1H are the same as those of the organic EL device 1F. The hole transporting zone 6H includes: the second mixture layer 66F between the first anode-side organic layer 61F and the second anode-side organic layer 62F; and the first mixture layer 65F between the second anode-side organic layer 62F and the third anode-side organic layer 63F. In the hole transporting zone 6H, the first anode-side organic layer 61F, the second mixture layer 66F, the second anode-side organic layer 62F, the first mixture layer 65F, and the third anode-side organic layer 63F are disposed in this order from a side close to the anode 3.

The invention is not limited to the exemplary arrangements of the organic EL devices depicted in FIGS. 7 to 9.

An organic EL device with another exemplary arrangement is exemplified by an organic EL device including the first emitting region 5A in place of the first emitting region 5 of the organic EL device depicted in FIG. 9.

Moreover, an organic EL device with still another exemplary arrangement is exemplified by an organic EL device in which the order of layering the emitting layers in the first emitting region 5A may be changed: the first emitting layer and the second emitting layer are layered in this order from a side close to the anode. Regardless of the order of the first emitting layer and the second emitting layer layered, the effect obtained by layering the first and second emitting layers in the first emitting region can be expected by selecting a combination of materials satisfying the relationship of the numerical formula (Numerical Formula 1).

Further, an organic EL device with a further exemplary arrangement may be, for instance, an organic EL device obtained by including the first mixture layer between the second anode-side organic layer 62F and the third anode-side organic layer 63F in the organic EL device depicted in each of FIGS. 7 and 8.

Furthermore, an organic EL device with a still further exemplary arrangement, for instance, an organic EL device obtained by including the second mixture layer between the first anode-side organic layer 61F and the second anode-side organic layer 62F in the organic EL device depicted in each of FIGS. 7 and 8.

### Additional Layers of Organic EL Device

The organic EL device of each exemplary embodiment may include one or more organic layers in addition to the above-described organic layer. The organic layer is exemplified by at least one layer selected from the group consisting of an interposed layer, a diffusion layer, a hole blocking layer, and an electron blocking layer.

### Interposed Layer

In an exemplary arrangement of the organic EL device of each exemplary embodiment, in a case where the first emitting region includes the first emitting layer and the second emitting layer, an interposed layer may be included as an organic layer disposed between the first emitting layer and the second emitting layer.

In an exemplary arrangement of the organic EL device of each exemplary embodiment, in order to inhibit an overlap between a Singlet emitting region and a TTF emitting region, the interposed layer contains no luminescent compound or may contain a luminescent compound in an insubstantial amount provided that the overlap can be inhibited.

For instance, the interposed layer contains 0 mass % of a luminescent compound. Alternatively, for instance, the interposed layer may contain a luminescent compound provided that the luminescent compound contained is a component accidentally mixed in a producing process or a component contained as impurities in a material.

For instance, when the interposed layer consists of a material A, a material B, and a material C, the content ratios of the materials A, B, and C in the interposed layer are each 10 mass % or more, and the total of the content ratios of the materials A, B, and C is 100 mass %.

In the following, the interposed layer is occasionally referred to as a “non-doped layer.” A layer containing a luminescent compound is occasionally referred to as a “doped layer.”

It is considered that luminous efficiency is improvable in an arrangement including layered emitting layers because the Singlet emitting region and the TTF emitting region are typically likely to be separated from each other.

In an exemplary arrangement of the organic EL device of each exemplary embodiment, in a case where the interposed layer (non-doped layer) is disposed between the first emitting layer and the second emitting layer in the first emitting region, it is expected that a region where the Singlet emitting region and the TTF emitting region overlap with each other is reduced to inhibit a decrease in TTF efficiency which may otherwise be caused by collision between triplet excitons and carriers. That is, it is considered that providing the interposed layer (non-doped layer) between the emitting layers contributes to the improvement in the efficiency of TTF emission.

The interposed layer is a non-doped layer. The interposed layer contains no metal atom. The interposed layer thus contains no metal complex. The interposed layer contains an interposed layer material. The interposed layer material is not a luminescent compound. The interposed layer material may be any material except for the luminescent compound.

Examples of the interposed layer material include: 1) a heterocyclic compound such as an oxadiazole derivative, benzimidazole derivative, or phenanthroline derivative; 2) a fused aromatic compound such as a carbazole derivative, anthracene derivative, phenanthrene derivative, pyrene derivative or chrysene derivative; and 3) an aromatic amine compound such as a triarylamine derivative or a fused polycyclic aromatic amine derivative.

One or both of the first host material and the second host material may be used as the interposed layer material. The interposed layer material may be any material provided that the Singlet emitting region and the TTF emitting region are separated from each other and the Singlet emission and the TTF emission are not hindered.

In an exemplary arrangement of the organic EL device of each exemplary embodiment, the respective content ratios of all the materials forming the interposed layer in the interposed layer are 10 mass % or more.

The interposed layer contains the interposed layer material as a material forming the interposed layer.

The interposed layer preferably contains 60 mass % or more of the interposed layer material, more preferably contains 70 mass % or more of the interposed layer material, still more preferably contains 80 mass % or more of the interposed layer material, still further more preferably 90 mass % or more of the interposed layer material, and yet still further more preferably 95 mass % or more of the interposed layer material, with respect to the total mass of the interposed layer.

The interposed layer may contain a single type of the interposed layer material or may contain two or more types of the interposed layer material.

When the interposed layer contains two or more types of the interposed layer material, the upper limit of the total of the content ratios of the two or more types of the interposed layer material is 100 mass %.

It should be noted that the interposed layer of each exemplary embodiment may further contain any other material than the interposed layer material.

The interposed layer may be provided in the form of a single layer or a laminate of two or more layers.

As long as the overlap between the Singlet emitting region and the TTF emitting region is inhibited, a film thickness of the interposed layer is not particularly limited but each layer in the interposed layer is preferably in a range from 3 nm to 15 nm, more preferably in a range from 5 nm to 10 nm.

The interposed layer having a film thickness of 3 nm or more easily separates the Singlet emitting region from the emitting region derived from TTF.

The interposed layer having a film thickness of 15 nm or less easily inhibits a phenomenon where the host material of the interposed layer emits light.

It is preferable that the interposed layer contains the interposed layer material as a material forming the interposed layer and the triplet energy of the first host material T₁(H1), the triplet energy of the second host material T₁(H2), and a triplet energy of at least one interposed layer material T₁(M_{mid}) satisfy a relationship of a numerical formula (Numerical Formula 21) below.

$${T}_{1} ( H 2 ) \geq {T}_{1} ( {M}_{mid} ) \geq {T}_{1} ( H 1 ) \qquad ( Numerical Formula 21 )$$

When the interposed layer contains two or more interposed layer materials as a material forming the interposed layer, the triplet energy of the first host material T₁(H1), the triplet energy of the second host material T₁(H2), and a triplet energy of each interposed layer material T₁(M_{EA}) more preferably satisfy a relationship of a numerical formula (Numerical Formula 21A) below.

$${T}_{1} ( H 2 ) \geq {T}_{1} ( {M}_{E A} ) \geq {T}_{1} ( H 1 ) \qquad ( Numerical Formula 21 A )$$

An exemplary arrangement of the organic EL device of each exemplary embodiment may further include a diffusion layer.

When an exemplary arrangement of the organic EL device of each exemplary embodiment includes the diffusion layer, the diffusion layer is preferably disposed between the first emitting layer and the second emitting layer.

The arrangement of the organic EL device will be further described below.

### Substrate

The substrate is used as a support for the organic EL device. For instance, glass, quartz, plastics and the like are usable for the substrate. A flexible substrate is also usable. The flexible substrate, which is a bendable substrate, is exemplified by a plastic substrate. Examples of a material for the plastic substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

The organic EL device of each exemplary embodiment may be a bottom emission type organic EL device. The organic EL device of each exemplary embodiment may be a top emission type organic EL device.

### Anode

Metal, an alloy, an electrically conductive compound, a mixture thereof, or the like having a large work function (specifically, 4.0 eV or more) is preferably used as the anode formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metallic material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

In a case of a bottom emission type organic EL device, the anode is preferably made of a light-transmissive or semi-transmissive metallic material that transmits light emitted from the emitting layer. Herein, the light-transmissive or semi-transmissive property means the property of allowing transmissivity of 50% or more (preferably 80% or more) of the light emitted from the emitting layer. The light-transmissive or semi-transmissive metallic material can be selected in use as needed from the above materials listed in the description regarding the anode.

In a case of a top emission type organic EL device, the anode is a reflective electrode having a reflective layer. The reflective layer is preferably made of a metallic material having light reflectivity. Herein, the light reflectivity means the property of reflecting 50% or more (preferably 80% or more) of the light emitted from the emitting layer. The metallic material having light reflectivity can be selected in use as needed from the above materials listed in the description about the anode.

The anode may consist of the reflective layer, but may have a multilayer structure having the reflective layer and a conductive layer (preferably a transparent conductive layer). In a case where the anode has the reflective layer and the conductive layer, the conductive layer is preferably disposed between the reflective layer and the hole transporting zone. A material of the conductive layer can be selected in use as needed from the above materials listed in the description regarding the anode.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method, and the like.

In a case of a bottom emission type organic EL device, the cathode is a reflective electrode. The reflective electrode is preferably made of a metallic material having light reflectivity. The metallic material having light reflectivity can be selected in use as needed from the above materials listed in the description regarding the cathode.

In a case of a top emission type organic EL device, the cathode is preferably made of a light-transmissive or semi-transmissive metallic material that transmits light emitted from the emitting layer. The light-transmissive or semi-transmissive metallic material can be selected in use as needed from the above materials listed in the description regarding the cathode.

### Capping Layer

In a case of a top emission type organic EL device, the organic EL device usually includes a capping layer on the top of the cathode.

The capping layer may contain, for instance, at least one compound selected from the group consisting of a high polymer compound, metal oxide, metal fluoride, metal boride, silicon nitride, and silicon compound (silicon oxide or the like).

The capping layer may contain, for instance, at least one compound selected from the group consisting of an aromatic amine derivative, an anthracene derivative, a pyrene derivative, a fluorene derivative, and a dibenzofuran derivative.

In addition, a laminate in which layers containing these substances are layered can also be used as the capping layer.

### Tandem Organic Electroluminescence Device

The organic EL device of each exemplary embodiment may be a so-called tandem organic EL device, in which a plurality of emitting regions are layered via a charge generating layer (occasionally also referred to as an intermediate layer or the like). The tandem organic EL device is exemplified by organic EL devices below.

An exemplary arrangement of the organic EL device of each exemplary embodiment includes: a first emitting unit including the first hole transporting zone and the first emitting region; a first charge generating layer disposed between the first emitting unit and the cathode; and a second emitting unit disposed between the first charge generating layer and the cathode. The second emitting unit includes a second hole transporting zone and a second emitting region. The first hole transporting zone, the first emitting region, the first charge generating layer, the second hole transporting zone, and the second emitting region are disposed in this order from a side close to the anode.

In an exemplary arrangement of the organic EL device of each exemplary embodiment, the tandem organic EL device further includes a third emitting unit and a second charge generating layer. The third emitting unit is disposed between the second emitting unit and the cathode, and the second charge generating layer is disposed between the third emitting unit and the second emitting unit. In an exemplary arrangement of organic EL device of each exemplary embodiment, the third emitting unit includes a third emitting region and a third hole transporting zone.

In the tandem organic EL devices with the above exemplary arrangements, the second emitting region and the third emitting region each independently include at least one emitting layer. The emitting layers included in the second emitting region and the third emitting region may be each independently the same as or different from the emitting layer included in the first emitting region.

In the tandem organic EL devices with these arrangements, the second hole transporting zone and the third hole transporting zone each independently include at least one organic layer. The organic layer included in the second hole transporting zone and the organic layer included in the third hole transporting zone may be each independently the same as or different from the organic layer included in the first hole transporting zone.

In the tandem organic EL devices with these arrangements, the first charge generating layer and the second charge generating layer each refer to a layer in which holes and electrons are generated when voltage is applied. For instance, when the first charge generating layer includes a plurality of layers, the first charge generating layer preferably includes an N layer disposed close to the anode and through which electrons are injected into the first emitting unit, and a P layer disposed close to the cathode and through which holes are injected into the second emitting unit. For instance, when the second charge generating layer includes a plurality of layers, the second charge generating layer preferably includes an N layer disposed close to the anode and through which electrons are injected into the second emitting unit, and a P layer disposed close to the cathode and through which holes are injected into the third emitting unit. Examples of a material usable for the first charge generating layer and the second charge generating layer include a known material(s) usable for the charge generating layer in the tandem organic EL device.

In an exemplary arrangement of the organic EL device of each exemplary embodiment, the tandem organic EL device is used for a light-emitting apparatus.

### Layer Formation Method

A method for forming each layer of the organic EL device according to any of the above exemplary embodiments is subject to no limitation except for the above particular description. However, known methods of dry film-forming such as vacuum deposition, sputtering, plasma or ion plating and wet film-forming such as spin coating, dipping, flow coating or ink-jet are applicable.

In an exemplary arrangement of the organic EL device of each exemplary embodiment, each layer of the organic EL device (e.g., each layer of the organic layer included in the first hole transporting zone and the emitting layer included in the emitting region) may be formed, for instance, by co-deposition using a plurality of types of compounds, by vapor-deposition using a mixture of a plurality of types of compounds prepared in advance, or by coating using a mixture of a plurality of types of compounds prepared in advance.

The mixture prepared by mixing a plurality of types of compounds in advance may be a powder. The mixture prepared by mixing a plurality of types of compounds in advance may be a solution. A method of mixing a plurality of types of compounds in advance is occasionally referred to as premix. A premix method, which is not particularly limited, enables adjustment of molecular weights of compounds forming a mixture by adjusting substituents or the like of the compounds or enables adjustment of a vapor-deposition ratio of compounds forming a mixture premixed.

The premixed mixture powder may contain a first organic compound and a second organic compound in one particle or may be a mixture of particles of the first organic compound and particles of the second organic compound.

As a method of producing the mixture powder, for instance, the first organic compound and the second organic compound may be pulverized and mixed using a mortar or the like, or the first organic compound and the second organic compound may be put into a container or the like and heated to be melted in a chemically inert environment, cooled to ambient temperature, and the resulting mixture is pulverized with a mixer or the like to form powder. According to the latter method, the first organic compound and the second organic compound can be mixed at a molecular level, whereby a ratio in term of a sublimation area between the first organic compound and the second organic compound can be easily controlled to fall within a desired range, which enables more uniform vapor-deposition. Moreover, it is possible to prevent disadvantages such as uneven mixing of mixture powder that may occur during delivery of the mixture powder.

Mixture powder may be compressed into pellets.

### Film Thickness

A film thickness of each layer of the organic layer of the organic EL device of each exemplary embodiment is not limited unless otherwise specified in the above. The film thickness of each layer of the organic layer of the organic EL device usually preferably ranges from several nanometers to 1 μm because in general an excessively small film thickness is likely to cause defects (e.g. pin holes) and an excessively large film thickness leads to the necessity of applying high voltage and consequent reduction in efficiency.

### Emission Wavelength of Organic EL Device

The organic EL device with an exemplary arrangement in each exemplary embodiment emits light whose maximum peak wavelength is 500 nm or less when the device is driven.

The organic EL device with an exemplary arrangement in each exemplary embodiment emits light whose maximum peak wavelength is in a range from 430 nm to 480 nm when the device is driven.

The maximum peak wavelength of the light emitted from the organic EL device when being driven is measured as follows. Voltage is applied to the organic EL device such that a current density is 10 mA/cm², where spectral radiance spectrum is measured by a spectroradiometer CS-2000 (produced by Konica Minolta, Inc.). A peak wavelength of an emission spectrum, a luminous intensity of which is the maximum in the obtained spectral radiance spectrum, is measured and defined as the maximum peak wavelength (unit: nm).

### Triplet Energy T₁

A method of measuring a triplet energy T₁ is exemplified by a method below.

A measurement target compound is dissolved in EPA (diethylether:
isopentane:ethanol=5:5:2 in volume ratio) so as to fall within a range from 10⁻⁵ mol/L to 10⁻⁴ mol/L to prepare a solution, and the obtained solution is encapsulated in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

$${T}_{1} [ eV ] = 1239 \text{.85} / {λ}_{e d g e} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. The measurement apparatus is not limited thereto. A combination of a cooling unit, a low temperature container, an excitation light source and a light-receiving unit may be used for measurement.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 104 mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge} (nm) at an intersection of the tangent and the abscissa axis is assigned to a conversion equation (F2) below to calculate singlet energy.

$${S}_{1} [ eV ] = 1239 \text{.85} / {λ}_{e d g e} \qquad Conversion Equation ( F2 )$$

Any apparatus for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Method of Measuring Electron Mobility

The electron mobility can be measured according to an impedance measurement using a mobility evaluation device produced by the following steps. The device for mobility evaluation is produced, for instance, according to the following steps.

A compound Target, which is to be measured for an electron mobility, is vapor-deposited on a glass substrate having an aluminum electrode (anode) so as to cover the aluminum electrode, thereby forming a measurement target layer. A compound ET-A below is vapor-deposited on this measurement target layer to form an electron transporting layer. LiF is vapor-deposited on this formed electron transporting layer to form an electron injecting layer. Metal aluminum (Al) is vapor-deposited on this formed electron injecting layer to form a metal cathode.

An arrangement of the mobility evaluation device above is roughly shown as follows.

$$glass / Al ( 50 ) / Target ( 200 ) / ET - A ( 10 ) / LiF ( 1 ) / Al ( 50 )$$

Numerals in parentheses represent a film thickness (nm).

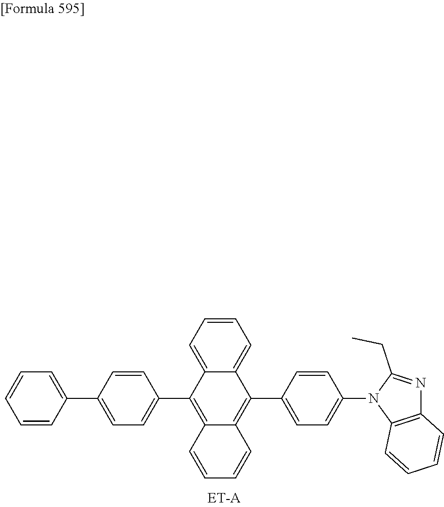 [SMILES: CCc1nc2ccccc2n1-c1ccc(-c2c3ccccc3c(-c3ccc(-c4ccccc4)cc3)c3ccccc23)cc1]

The mobility evaluation device for the electron mobility is set in an impedance measurement apparatus to perform an impedance measurement. In the impedance measurement, a measurement frequency is swept from 1 Hz to 1 MHz. At this time, an alternating current amplitude of 0.1 V and a direct current voltage V are simultaneously applied to the device. A modulus M is calculated from a measured impedance Z using a relationship of a calculation formula (C1) below.

$$M = j ωZ \qquad Calculation formula ( C1 )$$

In the calculation formula (C1), j is an imaginary unit whose square is −1 and ω is an angular frequency [rad/s].

In a bode plot in which an imaginary part of the modulus M is represented by an ordinate axis and the frequency [Hz] is represented by an abscissa axis, an electrical time constant T of the mobility evaluation device is obtained from a frequency fmax showing a peak using a calculation formula (C2) below.

$$τ = 1 / ( 2 π f max ) \qquad Calculation formula ( C2 )$$

IT in the calculation formula (C2) is a symbol representing a circumference ratio.

An electron mobility μe is calculated from a relationship of a calculation formula (C3-1) below using T.

$$μ e = {d}^{2} / ( V τ ) \qquad Calculation formula ( C3 )$$

d in the calculation formula (C3-1) is a total film thickness of organic thin film(s) forming the device. In a case of the arrangement of the mobility evaluation device for the electron mobility, d=210 [nm] is satisfied.

### Method of Measuring Hole Mobility

The hole mobility can be measured according to an impedance measurement using a mobility evaluation device produced by the following steps. The device for mobility evaluation is produced, for instance, according to the following steps.

A compound HA-2 below is vapor-deposited on a glass substrate having an ITO transparent electrode (anode) so as to cover the transparent electrode, thereby forming a hole injecting layer. A compound HT-A below is vapor-deposited on this formed hole injecting layer to form a hole transporting layer. Subsequently, a compound Target, which is to be measured for a hole mobility, is vapor-deposited to form a measurement target layer. Metal aluminum (Al) is vapor-deposited on this measurement target layer to form a metal cathode.

An arrangement of the mobility evaluation device above is roughly shown as follows.

$$ITO ( 1 \; 30 ) / HA - 2 ( 5 ) / HT - A ( 10 ) / Target ( 200 ) / Al ( 80 )$$

Numerals in parentheses represent a film thickness (nm).

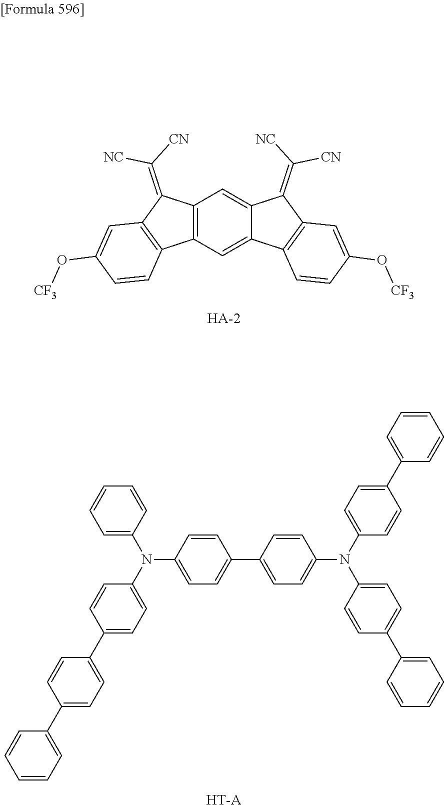 [SMILES: N#CC(C#N)=C1c2cc(OC(F)(F)F)ccc2-c2cc3c(cc21)C(=C(C#N)C#N)c1cc(OC(F)(F)F)ccc1-3 ; "HA-2" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "HT-A"]

The mobility evaluation device for the hole mobility is set in an impedance measurement apparatus to perform an impedance measurement. In the impedance measurement, a measurement frequency is swept from 1 Hz to 1 MHz. At this time, an alternating current amplitude of 0.1 V and a direct current voltage V are simultaneously applied to the device. A modulus M is calculated from a measured impedance Z using the relationship of the calculation formula (C1).

In a bode plot in which an imaginary part of the modulus M is represented by an ordinate axis and the frequency [Hz] is represented by an abscissa axis, an electrical time constant T of the mobility evaluation device is obtained from a frequency fmax showing a peak using the calculation formula (C2).

A hole mobility uh is calculated from a relationship of a calculation formula (C3-2) below using T obtained from the calculation formula (C2).

$$μ h = {d}^{2} / ( V τ ) \qquad Calculation formula ( C3 - 2 )$$

d in the calculation formula (C3-2) is a total film thickness of organic thin film(s) forming the device. In a case of the arrangement of the mobility evaluation device for the hole mobility, d=215 [nm] is satisfied.

The electron mobility and the hole mobility herein are each a value obtained in a case where a square root of an electric field intensity meets E^{1/2}=500 [V^{1/2}/cm^{1/2}]. The square root of the electric field intensity, E^{1/2}, can be calculated from a relationship of a calculation formula (C4) below.

$${E}^{1 / 2} = {V}^{1 / 2} / {d}^{1 / 2} \qquad Calculation formula ( C4 )$$

For the impedance measurement, a 1260 type by Solartron Analytical is used as the impedance measurement apparatus, and for a higher accuracy, a 1296 type dielectric constant measurement interface by Solartron Analytical can be used together therewith.

### Seventh Exemplary Embodiment

### Electronic Device

An electronic device according to a seventh exemplary embodiment is installed with any one of the organic EL devices according to any one of the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

In an exemplary arrangement of the electronic device according to the exemplary embodiment, the light-emitting apparatus is installed with any one of the tandem organic EL devices according to the above exemplary embodiments. In an exemplary arrangement of the electronic device according to the exemplary embodiment, the light-emitting apparatus preferably includes any one of the tandem organic EL devices according to the above exemplary embodiments and a color conversion layer. The light-emitting apparatus preferably includes a color filter. The color conversion layer is preferably disposed between the tandem organic EL device and the color filter. The color conversion layer preferably contains a substance that emits light by absorbing light. The substance that emits light by absorbing light is preferably a quantum dot. In the light-emitting apparatus, the color conversion layer is preferably disposed to be irradiated with light emission from the tandem organic EL device.

In an exemplary arrangement of the electronic device according to the exemplary embodiment, the display device is installed with the light-emitting apparatus of the exemplary embodiment. The light-emitting apparatus also can be used for a display device, for instance, as a backlight of the display device.

### Modification of Embodiment(s)

The scope of the invention is not limited by the above exemplary embodiments but includes any modification and improvement as long as such modification and improvement are compatible with the invention.

For instance, the number of emitting layers is not limited to one or two, and more than two emitting layers may be layered. For instance, the rest of the emitting layers may be a fluorescent emitting layer or a phosphorescent emitting layer with use of emission caused by electron transfer from the triplet excited state directly to the ground state.

The specific structure, shape, and the like of the components in the invention may be designed in any manner as long as an object of the invention can be achieved.

### EXAMPLES

The invention will be described in further detail with reference to Examples. The scope of the invention is by no means limited to Examples.

### Compounds

Structures of compounds used for forming the hole transporting zone of the organic EL device in each of Examples 1 to 16 and Comparatives 1 to 12 are shown below.

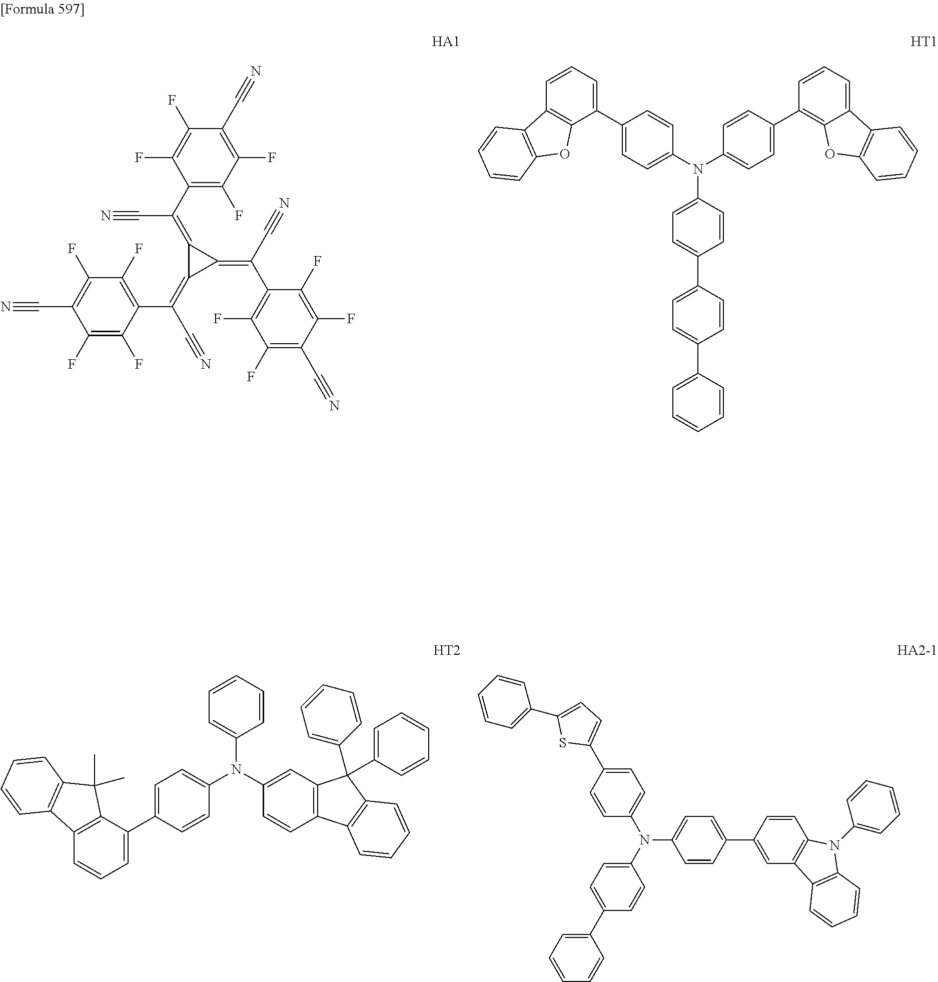 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; CC1(C)c2ccccc2-c2cccc(-c3ccc(N(c4ccccc4)c4ccc5c(c4)C(c4ccccc4)(c4ccccc4)c4ccccc4-5)cc3)c21 ; c1ccc(-c2ccc(N(c3ccc(-c4ccc5c(c4)c4ccccc4n5-c4ccccc4)cc3)c3ccc(-c4ccc(-c5ccccc5)s4)cc3)cc2)cc1]

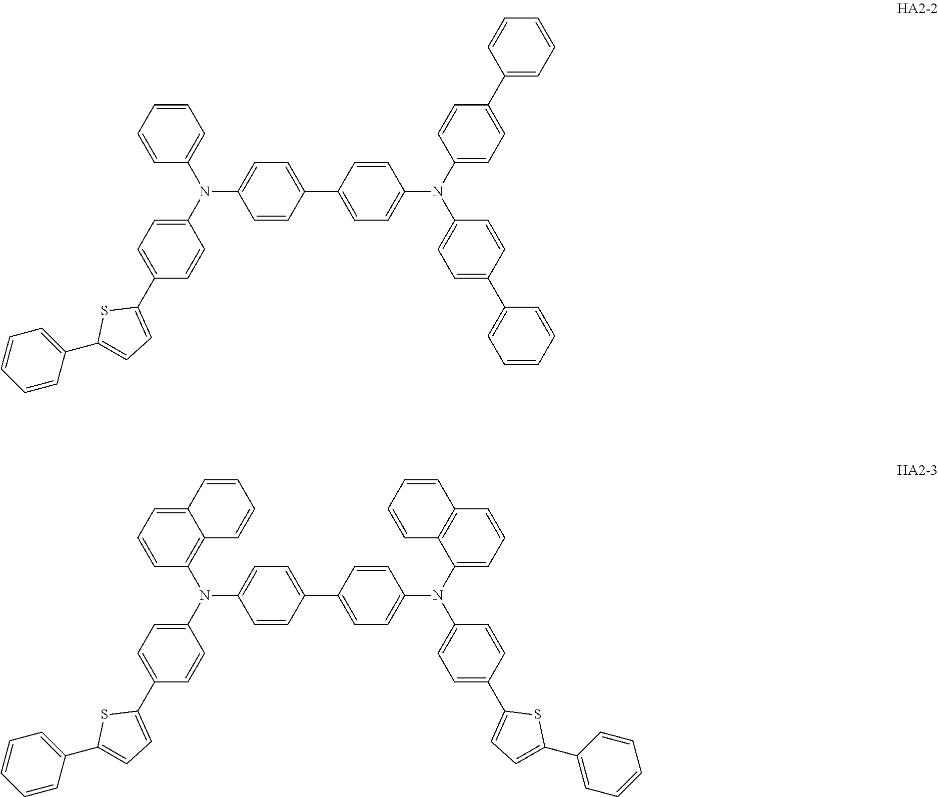 [SMILES: c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1 ; c1ccc(-c2ccc(-c3ccc(N(c4ccc(-c5ccc(N(c6ccc(-c7ccc(-c8ccccc8)s7)cc6)c6cccc7ccccc67)cc5)cc4)c4cccc5ccccc45)cc3)s2)cc1]

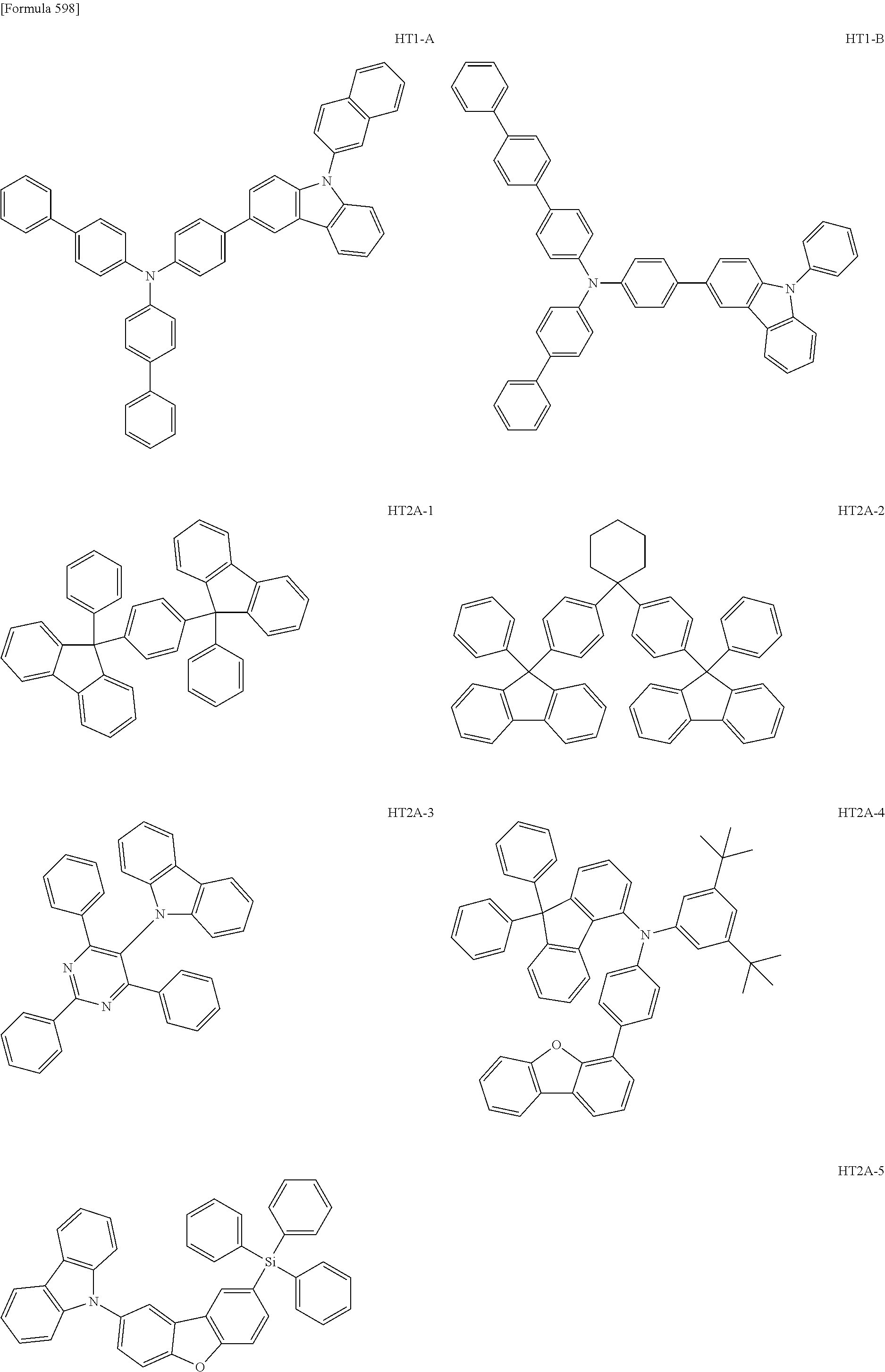

Structures of other compounds used for producing organic EL devices in Examples1 to 16 and Comparatives 1 to 12 are shown below.

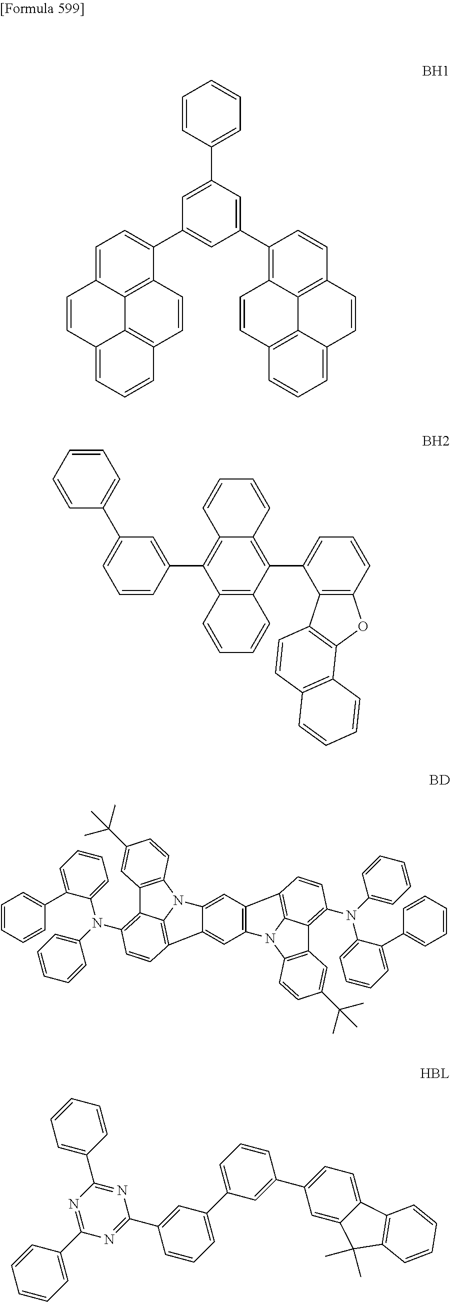

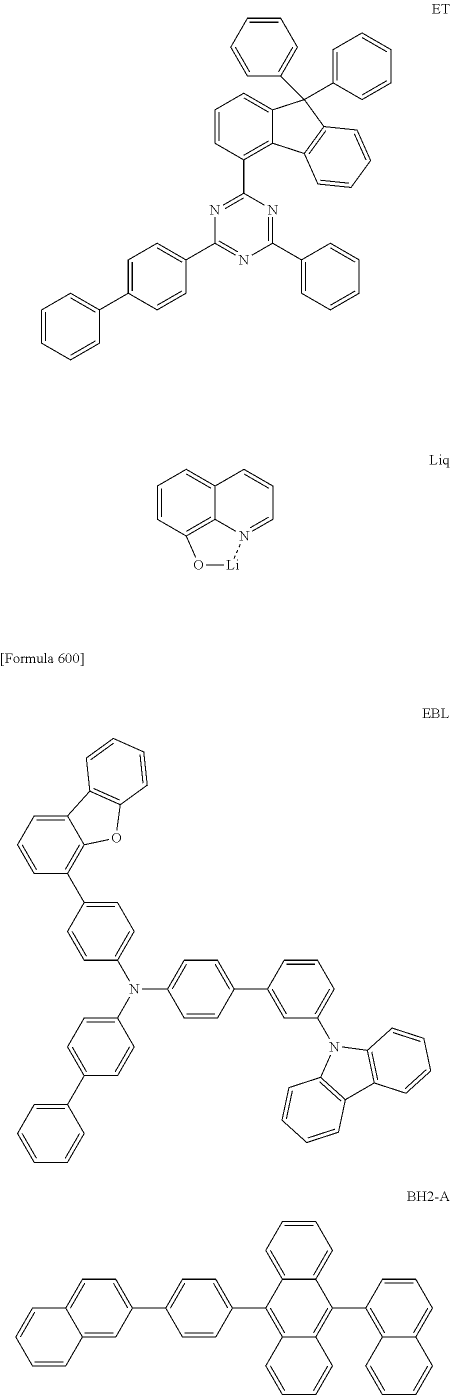

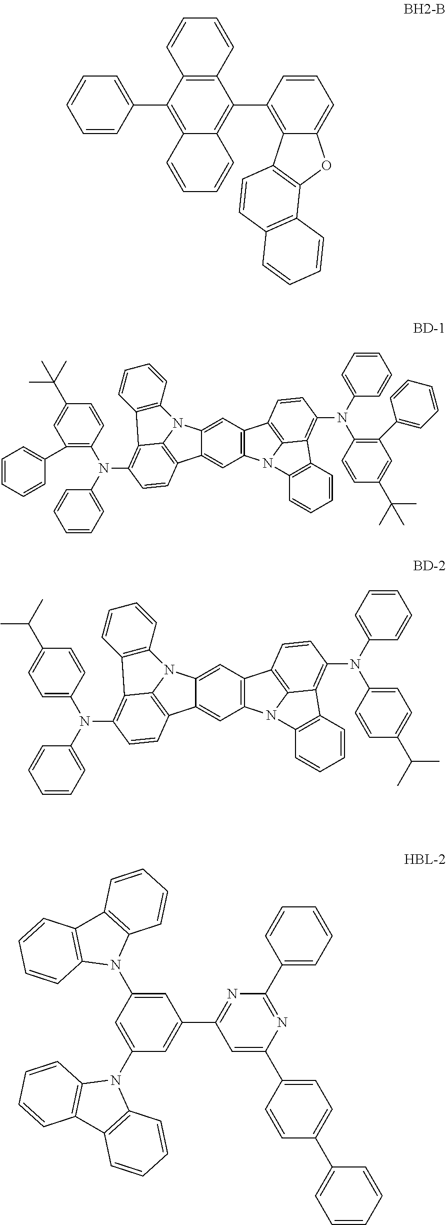 [SMILES: c1ccc(-c2c3ccccc3c(-c3cccc4oc5c6ccccc6ccc5c34)c3ccccc23)cc1 ; CC(C)(C)c1ccc(N(c2ccccc2)c2ccc3c4cc5c(cc4n4c6ccccc6c2c34)c2ccc(N(c3ccccc3)c3ccc(C(C)(C)C)cc3-c3ccccc3)c3c4ccccc4n5c23)c(-c2ccccc2)c1 ; CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

### Production (1) of Organic EL Device

Organic EL devices in Examples 1 to 3 as examples of the first exemplary embodiment or the second exemplary embodiment and organic EL devices in Comparatives 1 to 4 for comparison were produced as follows. The description regarding the first exemplary embodiment or the second exemplary embodiment is applied to the description regarding Examples 1 to 3 and Comparatives 1 to 4.

### Example 1

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, a compound HT1, a compound HA1, and a compound HA2-1 were co-deposited on a surface of the glass substrate, where the transparent electrode line was provided, to cover the transparent electrode, thereby forming a 10-nm-thick first anode-side organic layer. The ratios of the compound HT1, the compound HA1, and the compound HA2-1 in the first anode-side organic layer were 65 mass %, 10 mass %, and 25 mass %, respectively.

The compound HT2 was vapor-deposited on the first anode-side organic layer to form a 45-nm-thick second anode-side organic layer.

The compound BH1 (second host material) and the compound BD (second luminescent compound) were co-deposited on the second anode-side organic layer to form 10-nm-thick second emitting layer. The ratios of the compound BH1 and the compound BD in the second emitting layer were 99 mass % and 1 mass %, respectively.

A compound BH2 (first host material) and the compound BD (first luminescent compound) were co-deposited on the second emitting layer to form a 10-nm-thick first emitting layer. The ratios of the compound BH2 and the compound BD in the first emitting layer were 99 mass % and 1 mass %, respectively.

A compound HBL was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 1 is roughly shown as follows.

$$ITO ( 130 ) / HT 1 : HA 1 : HA 2 - 1 ( 10 , 65 \% : 10 \% : 25 \% ) / \\ HT 2 ( 45 ) / BH 1 : BD ( 10 , 99 \% : 1 \% ) / BH 2 : BD ( 10 , 99 \% : 1 \% ) / \\ HBL ( 5 ) / ET : Liq ( 25 , 50 \% : 50 \% ) / Yb ( 1 ) / Al ( 80 )$$

Numerals in parentheses represent a film thickness (unit: nm).

Regarding the device arrangement in Example 1: the numerals (65%: 10%: 25%) represented by percentage in the same parentheses indicate a ratio (mass %) between the compound HT1, the compound HA1, and the compound HA2-1 in the first anode-side organic layer; the numerals (99%: 1%) represented by percentage in the same parentheses indicate a ratio (mass %) between the compound BH1 or the compound BH2 and the compound BD in the first emitting layer or the second emitting layer; and the numerals (50%: 50%) represented by percentage in the same parentheses indicate a ratio (mass %) between the compound ET and the compound Liq in the second electron transporting layer (ET). Similar notations apply to the description below.

### Examples 2 and 3

Organic EL devices in Examples 2 to 3 were each produced in the same manner as the organic EL device in Example 1 except that the compound HA2-1 as the additive material (fourth material) in the first anode-side organic layer was replaced with additive materials listed in Table 1.

### Comparative 1

An organic EL device in Comparative 1 was produced in the same manner as the organic EL device in Example 1 except that the compound used for forming the first anode-side organic layer was replaced with the compound HT1 and the compound HA1. The ratios of the compound HT1 and the compound HA1 in the first anode-side organic layer in Comparative 1 were 90 mass % and 10 mass %, respectively.

### Comparative 2

An organic EL device in Comparative 2 was produced in the same manner as the organic EL device in Example 1 except that the compound used for forming the first anode-side organic layer was replaced with the compound HA2-1 and the compound HA1. The ratios of the compound HA2-1 and the compound HA1 in the first anode-side organic layer in Comparative 2 were 90 mass % and 10 mass %, respectively.

### Comparatives 3 and 4

Organic EL devices in Comparatives 3 to 4 were each produced in the same manner as the organic EL device in Comparative 2 except that the compound HA2-1 as the additive material (fourth material) in the first anode-side organic layer was replaced with additive materials listed in Table 1.

### Evaluation (1) of Organic EL Devices

The organic EL devices produced in Examples 1 to 3 and Comparatives 1 to 4 were evaluated as follows. Table 1 shows the evaluation results.

### External Quantum Efficiency EQE

Voltage was applied to the produced organic EL devices such that a current density was 10 mA/cm², where spectral radiance spectrum was measured by a spectroradiometer CS-2000 (produced by Konica Minolta, Inc.). The external quantum efficiency EQE was calculated based on the obtained spectral radiance spectra, assuming that the spectra was provided under a Lambertian radiation. A unit of EQE is %. Table 1 shows relative values of EQE, which were calculated according to a numerical formula (Numerical Formula X1) below. A unit of the relative value of EQE is %.

$$EQE ( relative value ) = ( EQE of each \\ Example / EQE of Comparative 1 ) \times 100 \qquad ( Numerical Formula X1 )$$

### Lifetime LT95

Voltage was applied to the produced organic EL device such that a current density was 50 mA/cm², where a time (LT95 (unit: hr)) elapsed before a luminance intensity was reduced to 95% of the initial luminance intensity was measured as a lifetime. The luminance intensity was measured with a spectroradiometer CS-2000 (manufactured by Konica Minolta, Inc.). Relative values of lifetime LT95 were calculated based on the measurement value of LT95 in each Example according to a numerical formula (Numerical Formula X₂) below. A unit of the relative value of lifetime LT95 is %.

$$LT 95 ( relative value ) = ( LT 95 of each \\ Example / LT 95 of Comparative 1 ) \times 100 \qquad ( Numerical Formula X2 )$$

TABLE 1

| | | | | | | | | | | Second anode-side | | | | |
|---|---|---|---|---|---|---|---|---|---|---|---|---|---|---|
| | First anode-side organic layer | | | | | | | | | organic layer | | | Device evaluation | |
| | First organic material | | | Second organic | | Additive material | | Refrac- | Film | Third | Refrac- | Film | result | |
| | | Refrac- | | material | | (Fourth material) | | tive | thick- | organic | tive | thick- | EQE | LT95 |
| | | tive | Content | | Content | | Content | index | ness | material | index | ness | (relative | (relative |
| | Name | index | [mass %] | Name | [mass %] | Name | [mass %] | NM₁ | [nm] | Name | NM₂ | [nm] | value) | value) |
| Ex. 1 | HT1 | 2.03 | 65 | HA1 | 10 | HA2-1 | 25 | 2.05 | 10 | HT2 | 1.79 | 45 | 103% | 100% |
| Ex. 2 | HT1 | 2.03 | 65 | HA1 | 10 | HA2-2 | 25 | 2.04 | 10 | HT2 | 1.79 | 45 | 102% | 102% |
| Ex. 3 | HT1 | 2.03 | 65 | HA1 | 10 | HA2-3 | 25 | 2.05 | 10 | HT2 | 1.79 | 45 | 102% | 102% |
| Comp. 1 | HT1 | 2.03 | 90 | HA1 | 10 | — | — | 2.03 | 10 | HT2 | 1.79 | 45 | 100% | 100% |
| Comp. 2 | — | — | — | HA1 | 10 | HA2-1 | 90 | 2.10 | 10 | HT2 | 1.79 | 45 | 101% | 80% |
| Comp. 3 | — | — | — | HA1 | 10 | HA2-2 | 90 | 2.08 | 10 | HT2 | 1.79 | 45 | 101% | 75% |
| Comp. 4 | — | — | — | HA1 | 10 | HA2-3 | 90 | 2.09 | 10 | HT2 | 1.79 | 45 | 93% | 70% |

The organic EL devices of Examples 1 to 3 each had an improved EQE as compared with the organic EL device of Comparative 1. The organic EL devices of Examples 2 to 3 each had a prolonged lifetime as compared with the organic EL device of Comparative 1. The organic EL devices of Comparatives 2 to 4, in which the first anode-side organic layer contained only the second organic material and the additive material (fourth material), exhibited a large refractive index NM₁ of the first anode-side organic layer but had a shortened lifetime.

### Production (2) of Organic EL Device

An organic EL devices in Example 4 as an example of the first exemplary embodiment or the third exemplary embodiment and an organic EL device in Comparative 5 for comparison were produced as follows. The description regarding the first exemplary embodiment or the third exemplary embodiment is applied to the description regarding Example 4 and Comparative 5.

### Example 4

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, the compound HT1-A and the compound HA1 were co-deposited on a surface of the glass substrate, where the transparent electrode line was provided, to cover the transparent electrode, thereby forming a 10-nm-thick first anode-side organic layer. The ratios of the compound HT1-A and the compound HA1 in the first anode-side organic layer were 97 mass % and 3 mass %, respectively.

The compound HT2 and the compound HT2A-1 were co-deposited on the first anode-side organic layer to form a 45-nm-thick second anode-side organic layer. The ratios of the compound HT2 and the compound HT2A-1 in the second anode-side organic layer were both 50 mass %.

The compound EBL was vapor-deposited on the second anode-side organic layer to form a 5-nm-thick fourth anode-side organic layer.

A compound BH2-A (first host material) and a compound BD-1 (first luminescent compound) were co-deposited on the fourth anode-side organic layer to form a 20-nm-thick first emitting layer. The ratios of the compound BH2-A and the compound BD-1 in the first emitting layer were 99 mass % and 1 mass %, respectively.

A compound HBL-2 was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 4 is roughly shown as follows.

$$ITO ( 130 ) / HT 1 - A : HA 1 ( 10 , 97 \% : 3 \% ) / \\ HT 2 : HT 2 A - 1 ( 45 , 50 \% : 50 \% ) / EBL ( 5 ) / \\ BH 2 - A : BD - 1 ( 20 , 99 \% : 1 \% ) / HBL - 2 ( 5 ) / \\ ET : Liq ( 25 , 50 \% : 50 \% ) / Yb ( 1 ) / Al ( 80 )$$

### Comparative 5

An organic EL device of Comparative 5 was produced in the same manner as the organic EL device of Example 4 except that the second anode-side organic layer was formed using only the compound HT2 without using the fifth material (compound HT2A-1) as the additive material. The ratio of the compound HT2 in the second anode-side organic layer in Comparative 5 was 100 mass %.

### Evaluation (2) of Organic EL Devices

The organic EL devices produced in Example 4 and Comparative 5 were evaluated as follows. Table 2 shows the evaluation results.

### External Quantum Efficiency EQE

External quantum efficiency EQE of each of the produced organic EL devices was calculated in the same manner as in <Evaluation (1) of Organic EL Devices>. Table 2 shows relative values of EQE, which were calculated according to a numerical formula (Numerical Formula X11) below.

$$EQE ( relative value ) = ( EQE of each \\ Example / EQE of Comparative 5 ) \times 100 \qquad ( Numerical Formula X11 )$$

### Lifetime LT95

Lifetime LT95 of each of the produced organic EL devices was measured in the same manner as in <Evaluation (1) of Organic EL Devices>. Relative values of lifetime LT95 were calculated based on the measurement value of LT95 in each Example according to a numerical formula (Numerical Formula X21) below.

$$LT 95 ( relative value ) = ( LT 95 of each \\ Example / LT 95 of Comparative 5 ) \times 100 \qquad ( Numerical Formula X21 )$$

TABLE 2

| | | | | | | | | Second anode- |
|---|---|---|---|---|---|---|---|---|
| | First anode-side organic layer | | | | | | | side organic |
| | | | | Second organic | | | | layer |
| | First organic material | | | material | | Refractive | Film | Third organic |
| | | Refractive | Content | | Content | index | thickness | material |
| | Name | index | [mass %] | Name | [mass %] | NM₁ | [nm] | Name |
| Ex. 4 | HT1-A | 1.97 | 97 | HA1 | 3 | 1.97 | 10 | HT2 |
| Comp. 5 | HT1-A | 1.97 | 97 | HA1 | 3 | 1.97 | 10 | HT2 |

| | | | | | | | | Device |
|---|---|---|---|---|---|---|---|---|
| | Second anode-side organic layer | | | | | | | evaluation |
| | | | Additive material | | | | | result |
| | Third organic material | | (Fifth material) | | | Refractive | Film | EQE |
| | Refractive | Content | | Refractive | Content | index | thickness | (relative |
| | index | [mass %] | Name | index | [mass %] | NM₂ | [nm] | value) |
| Ex. 4 | 1.79 | 50 | HT2A-1 | 1.73 | 50 | 1.76 | 45 | 106% |
| Comp. 5 | 1.79 | 100 | — | — | — | 1.79 | 45 | 100% |

Due to containing the additive material (fifth material) in the second anode-side organic layer, the organic EL device of Example 4 had an improved EQE as compared with the organic EL device of Comparative 5.

### Production (3) of Organic EL Device

Organic EL devices in Examples 5 to 8 as examples of the first exemplary embodiment or the third exemplary embodiment and an organic EL device in Comparative 6 for comparison were produced as follows. The description regarding the first exemplary embodiment or the third exemplary embodiment is applied to the description regarding Examples 5 to 8 and Comparative 6.

### Example 5

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, the compound HT1-B and the compound HA1 were co-deposited on a surface of the glass substrate, where the transparent electrode line was provided, to cover the transparent electrode, thereby forming a 10-nm-thick first anode-side organic layer. The ratios of the compound HT1-B and the compound HA1 in the first anode-side organic layer were 97 mass % and 3 mass %, respectively.

The compound HT2 and a compound HT2A-2 were co-deposited on the first anode-side organic layer to form a 40-nm-thick second anode-side organic layer. The ratios of the compound HT2 and the compound HT2A-2 in the second anode-side organic layer were both 50 mass %.

The compound EBL was vapor-deposited on the second anode-side organic layer to form a 5-nm-thick fourth anode-side organic layer.

A compound BH2-B (first host material) and a compound BD-2 (first luminescent compound) were co-deposited on the fourth anode-side organic layer to form a 20-nm-thick first emitting layer. The ratios of the compound BH2-B and the compound BD-2 in the first emitting layer were 99 mass % and 1 mass %, respectively.

A compound HBL was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 5 is roughly shown as follows.

$$ITO ( 130 ) / HT 1 - B : HA 1 ( 10 , 97 \% : 3 \% ) / \\ HT 2 : HT 2 A - 2 ( 40 , 50 \% : 50 \% ) / EBL ( 5 ) / \\ BH 2 - B : BD - 2 ( 20 , 99 \% : 1 \% ) / HBL ( 5 ) / \\ ET : Liq ( 25 , 50 \% : 50 \% ) / Yb ( 1 ) / Al ( 80 )$$

### Example 6

An organic EL device of Example 6 was produced in the same manner as the organic EL device in Example 5 except that the compound HT2A-2 as the additive material (fifth material) in the second anode-side organic layer was replaced with the additive material listed in Table 3.

### Example 7

An organic EL device of Example 7 was produced in the same manner as the organic EL device in Example 5 except that the compound HT2A-2 as the additive material (fifth material) in the second anode-side organic layer was replaced with the additive material (compound HT2A-4) listed in Table 3, and the ratios of the compound HT2 and the compound HT2A-2 in the second anode-side organic layer were changed to 70 mass % and 30 mass %, respectively.

### Example 8

An organic EL device of Example 8 was produced in the same manner as the organic EL device in Example 5 except that the compound HT2A-2 as the additive material (fifth material) in the second anode-side organic layer was replaced with the additive material listed in Table 3.

### Comparative 6

An organic EL device of Comparative 6 was produced in the same manner as the organic EL device of Example 5 except that the second anode-side organic layer was formed using only the compound HT2 without using the fifth material as the additive material. The ratio of the compound HT2 in the second anode-side organic layer in Comparative 6 was 100 mass %.

### Evaluation (3) of Organic EL Devices

The organic EL devices produced in Examples 5 to 8 and Comparative 6 were evaluated as follows. Table 3 shows the evaluation results.

### External Quantum Efficiency EQE

External quantum efficiency EQE of each of the produced organic EL devices was calculated in the same manner as in <Evaluation (1) of Organic EL Devices>. Table 3 shows relative values of EQE, which were calculated according to a numerical formula (Numerical Formula X₁₂) below.

$$EQE ( relative value ) = ( EQE of each \\ Example / EQE of Comparative 6 ) \times 100 \qquad ( Numerical Formula X12 )$$

### Lifetime LT95

Lifetime LT95 of each of the produced organic EL devices was measured in the same manner as in <Evaluation (1) of Organic EL Devices>. Relative values of lifetime LT95 were calculated based on the measurement value of LT95 in each Example according to a numerical formula (Numerical Formula X₂₂) below.

$$LT 95 ( relative value ) = ( LT 95 of each \\ Example / LT 95 of Comparative 6 ) \times 100 \qquad ( Numerical Formula X22 )$$

TABLE 3

| | First anode-side organic layer | | | | | | | | |
|---|---|---|---|---|---|---|---|---|---|
| | | | | Second organic | | | | Second anode-side organic layer | |
| | First organic material | | | material | | Refractive | Film | Third organic material | |
| | | Refractive | Content | | Content | index | thickness | | Refractive |
| | Name | index | [mass %] | Name | [mass %] | NM₁ | [nm] | Name | index |
| Ex. 5 | HT1-B | 1.99 | 97 | HA1 | 3 | 1.99 | 10 | HT2 | 1.79 |
| Ex. 6 | HT1-B | 1.99 | 97 | HA1 | 3 | 1.99 | 10 | HT2 | 1.79 |
| Ex. 7 | HT1-B | 1.99 | 97 | HA1 | 3 | 1.99 | 10 | HT2 | 1.79 |
| Ex. 8 | HT1-B | 1.99 | 97 | HA1 | 3 | 1.99 | 10 | HT2 | 1.79 |
| Comp. 6 | HT1-B | 1.99 | 97 | HA1 | 3 | 1.99 | 10 | HT2 | 1.79 |

| | | | | | | | Device |
|---|---|---|---|---|---|---|---|
| | Second anode-side organic layer | | | | | | evaluation |
| | Third organic | Additive material | | | | | result |
| | material | (Fifth material) | | | Refractive | Film | EQE |
| | Content | | Refractive | Content | index | thickness | (relative |
| | [mass %] | Name | index | [mass %] | NM₂ | [nm] | value) |
| Ex. 5 | 50 | HT2A-2 | 1.76 | 50 | 1.78 | 40 | 106% |
| Ex. 6 | 50 | HT2A-3 | 1.53 | 50 | 1.66 | 40 | 104% |
| Ex. 7 | 70 | HT2A-4 | 1.76 | 30 | 1.78 | 40 | 105% |
| Ex. 8 | 50 | HT2A-5 | 1.76 | 50 | 1.78 | 40 | 104% |
| Comp. 6 | 100 | — | — | — | 1.79 | 40 | 100% |

Due to containing the additive material (fifth material) in the second anode-side organic layer, the organic EL devices of Examples 5 to 8 had an improved EQE as compared with the organic EL device of Comparative 6.

### Production (4) of Organic EL Device

Organic EL devices in Examples 9 to 11 as examples of the fourth exemplary embodiment or the fifth exemplary embodiment and organic EL devices in Comparatives 7 to 10 for comparison were produced as follows. The description regarding the fourth exemplary embodiment or the fifth exemplary embodiment is applied to the description regarding Examples 9 to 11 and Comparatives 7 to 10.

### Example 9

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, the compound HT1 and the compound HA1 were co-deposited on a surface of the glass substrate, where the transparent electrode line was provided, to cover the transparent electrode, thereby forming a 10-nm-thick first anode-side organic layer. The ratios of the compound HT1 and the compound HA1 in the first anode-side organic layer were 90 mass % and 10 mass %, respectively.

The compound HT1 and a compound HA2-1 were co-deposited on the first anode-side organic layer to form a 40-nm-thick second anode-side organic layer. The ratios of the compound HT1 and the compound HA2-1 in the second anode-side organic layer were 75 mass % and 25 mass %, respectively.

The compound HT2 was vapor-deposited on the second anode-side organic layer to form a 45-nm-thick third anode-side organic layer.

The compound BH1 (second host material) and the compound BD (second luminescent compound) were co-deposited on the third anode-side organic layer to form 10-nm-thick second emitting layer. The ratios of the compound BH1 and the compound BD in the second emitting layer were 99 mass % and 1 mass %, respectively.

A compound BH2 (first host material) and the compound BD (first luminescent compound) were co-deposited on the second emitting layer to form a 10-nm-thick first emitting layer. The ratios of the compound BH2 and the compound BD in the first emitting layer were 99 mass % and 1 mass %, respectively.

A compound HBL was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 9 is roughly shown as follows.

$$ITO ( 130 ) / HT 1 : HA 1 ( 10 , 90 \% : 10 \% ) / HT 1 : HA 2 - 1 ( 40 , 75 \% : 25 \% ) / \\ ( HT 2 ( 45 ) / BH 1 : BD ( 10 , 99 \% : 1 \% ) / BH 2 : BD ( 10 , 99 \% : 1 \% ) / \\ HBL ( 5 ) / ET : Liq ( 25 , 50 \% : 50 \% ) / Yb ( 1 ) / Al ( 80 )$$

Numerals in parentheses represent a film thickness (unit: nm).

Regarding the device arrangement in Example 9: the numerals (90:10%) represented by percentage in the same parentheses indicate a ratio (mass %) between the compound HT1 and the compound HA1 in the first anode-side organic layer; the numerals (75:25%) represented by percentage in the same parentheses indicate a ratio (mass %) between the compound HT1 and the compound HA2-1 in the second anode-side organic layer; the numerals (99%: 1%) represented by percentage in the same parentheses indicate a ratio (mass %) between the compound BH1 or the compound BH2 and the compound BD in the first emitting layer or the second emitting layer; and the numerals (50%: 50%) represented by percentage in the same parentheses indicate a ratio (mass %) between the compound ET and the compound Liq in the second electron transporting layer (ET). Similar notations apply to the description below.

### Examples 10 and 11

Organic EL devices in Examples 10 and 11 were each produced in the same manner as the organic EL device in Example 9 except that the compound HA2-1 as the additive material (fourth material) in the second anode-side organic layer was replaced with the additive material listed in Table 4.

### Comparative 7

An organic EL device in Comparative 7 was produced in the same manner as the organic EL device in Example 9 except that the compound used for forming the second anode-side organic layer was replaced with the compound HT1 and no additive material was used.

### Comparative 8

An organic EL device in Comparative 8 was produced in the same manner as the organic EL device in Example 9 except that the compound used for forming the second anode-side organic layer was replaced with the compound HA2-1 and the compound HT1 was not used.

### Comparatives 9 and 10

Organic EL devices in Comparatives 9 and 10 were each produced in the same manner as the organic EL device in Comparative 8 except that the compound HA2-1 as the additive material (fourth material) in the second anode-side organic layer was replaced with the additive materials listed in Table 4.

### Evaluation (4) of Organic EL Devices

The organic EL devices produced in Examples 9 to 11 and Comparatives 7 to 10 were evaluated as follows. Table 4 shows the evaluation results.

### External Quantum Efficiency EQE

Voltage was applied to the produced organic EL devices such that a current density was 10 mA/cm², where spectral radiance spectrum was measured by a spectroradiometer CS-2000 (produced by Konica Minolta, Inc.). The external quantum efficiency EQE was calculated based on the obtained spectral radiance spectra, assuming that the spectra was provided under a Lambertian radiation. A unit of EQE is %. Table 4 shows relative values of EQE, which were calculated according to a numerical formula (Numerical Formula X13) below. A unit of the relative value of EQE is %.

$$EQE ( relative value ) = EQE of each Example / EQE of Comparative 7 ) \times 100 \qquad ( Numerical Formula X13 )$$

### Lifetime LT95

Voltage was applied to the produced organic EL device such that a current density was 50 mA/cm², where a time (LT95 (unit: hr)) elapsed before a luminance intensity was reduced to 95% of the initial luminance intensity was measured as a lifetime. The luminance intensity was measured with a spectroradiometer CS-2000 (manufactured by Konica Minolta, Inc.). Relative values of lifetime LT95 were calculated based on the measurement value of LT95 in each Example according to a numerical formula (Numerical Formula X₂₃) below. A unit of the relative value of lifetime LT95 is %.

LT95 (relative value)=(LT95 of each Example/LT95 of Comparative 7)× 100 (Numerical Formula X23)

TABLE 4

| | | | | | | | | | | | Third anode-side | | | |
|---|---|---|---|---|---|---|---|---|---|---|---|---|---|---|
| | First anode-side organic layer | | | | | Second anode-side organic layer | | | | | organic layer | | Device evaluation | |
| | First organic | | | | Refrac- | Second organic | | Additive material | | Refrac- | Third | Refrac- | result | |
| | material | | Acceptor material | | tive | material | | (Fourth material) | | tive | organic | tive | EQE | LT95 |
| | | Content | | Content | index | | Content | | Content | index | material | index | (relative | (relative |
| | Name | [mass %] | Name | [mass %] | NM₁ | Name | [mass %] | Name | [mass %] | NM₂ | Name | NM₃ | value) | value) |
| Ex. 9 | HT1 | 90 | HA1 | 10 | 2.03 | HT1 | 75 | HA2-1 | 25 | 2.05 | HT2 | 1.79 | 103% | 100% |
| Ex. 10 | HT1 | 90 | HA1 | 10 | 2.03 | HT1 | 75 | HA2-2 | 25 | 2.04 | HT2 | 1.79 | 102% | 102% |
| Ex. 11 | HT1 | 90 | HA1 | 10 | 2.03 | HT1 | 75 | HA2-3 | 25 | 2.05 | HT2 | 1.79 | 102% | 102% |
| Comp. 7 | HT1 | 90 | HA1 | 10 | 2.03 | HT1 | 100 | — | — | 2.03 | HT2 | 1.79 | 100% | 100% |
| Comp. 8 | HT1 | 90 | HA1 | 10 | 2.03 | — | — | HA2-1 | 100 | 2.10 | HT2 | 1.79 | 101% | 70% |
| Comp. 9 | HT1 | 90 | HA1 | 10 | 2.03 | — | — | HA2-2 | 100 | 2.08 | HT2 | 1.79 | 101% | 65% |
| Comp. 10 | HT1 | 90 | HA1 | 10 | 2.03 | — | — | HA2-3 | 100 | 2.09 | HT2 | 1.79 | 93% | 60% |

The organic EL devices of Examples 9 to 11 each had an improved EQE as compared with the organic EL device of Comparative 7. The organic EL devices of Examples 10 to 11 each had a prolonged lifetime as compared with the organic EL device of Comparative 7. The organic EL devices of Comparatives 8 to 10, in which the second anode-side organic layer contained only the additive material (fourth material), exhibited a large refractive index NM₂ of the second anode-side organic layer but had a shortened lifetime.

### Production (5) of Organic EL Device

An organic EL device in Example 12 as an example of the fourth exemplary embodiment or the sixth exemplary embodiment and an organic EL device in Comparative 11 for comparison were produced as follows. The description regarding the fourth exemplary embodiment or the sixth exemplary embodiment is applied to the description regarding Example 12 and Comparative 11.

### Example 12

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, the compound HT1-A and the compound HA1 were co-deposited on a surface of the glass substrate, where the transparent electrode line was provided, to cover the transparent electrode, thereby forming a 10-nm-thick first anode-side organic layer. The ratios of the compound HT1-A and the compound HA1 in the first anode-side organic layer were 97 mass % and 3 mass %, respectively.

The compound HT1-A was vapor-deposited on the first anode-side organic layer to form a 35-nm-thick second anode-side organic layer.

The compound HT2 and the compound HT2A-1 were co-deposited on the second anode-side organic layer to form a 45-nm-thick third anode-side organic layer. The ratios of the compound HT2 and the compound HT2A-1 in the third anode-side organic layer were both 50 mass %.

The compound EBL was vapor-deposited on the third anode-side organic layer to form a 5-nm-thick fourth anode-side organic layer.

A compound BH2-A (first host material) and a compound BD-1 (first luminescent compound) were co-deposited on the fourth anode-side organic layer to form a 20-nm-thick first emitting layer. The ratios of the compound BH2-A and the compound BD-1 in the first emitting layer were 99 mass % and 1 mass %, respectively.

A compound HBL-2 was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 12 is roughly shown as follows.

$$ITO ( 130 ) / HT 1 - A : HA 1 ( 10 , 97 \% : 3 \% ) / HT 1 - A ( 35 ) / HT 2 : HT 2 A - 1 ( 45 , 50 \% : 50 \% ) / EBL ( 5 ) / BH 2 - A : BD - 1 ( 20 , 99 \% : 1 \% ) / HBL - 2 ( 5 ) / ET : Liq ( 25 , 50 \% : 50 \% ) / Yb ( 1 ) / Al ( 80 )$$

### Comparative 11

An organic EL device of Comparative 11 was produced in the same manner as the organic EL device of Example 12 except that the third anode-side organic layer was formed using only the compound HT2 without using the fifth material as the additive material. The ratio of the compound HT2 in the third anode-side organic layer in Comparative 11 was 100 mass %.

### Evaluation (5) of Organic EL Devices

The organic EL devices produced in Example 12 and Comparative 11 were evaluated as follows. Table 5 shows the evaluation results.

### External Quantum Efficiency EQE

External quantum efficiency EQE of each of the produced organic EL devices was calculated in the same manner as in <Evaluation (4) of Organic EL Devices>. Table 5 shows relative values of EQE, which were calculated according to a numerical formula (Numerical Formula X₁₄) below.

$$EQE ( relative value ) = EQE of each Example / EQE of Comparative 11 ) \times 100 \qquad ( Numerical Formula X14 )$$

### Lifetime LT95

Lifetime LT95 of each of the produced organic EL devices was measured in the same manner as in <Evaluation (4) of Organic EL Devices>. Relative values of lifetime LT95 were calculated based on the measurement value of LT95 in each Example according to a numerical formula (Numerical Formula X24) below.

$$LT 95 ( relative value ) = ( LT 95 of each Example / LT 95 of Comparative 11 ) \times 100 \qquad ( Numerical Formula X24 )$$

TABLE 5

| | | | | | Second anode-side | | Third anode-side | |
|---|---|---|---|---|---|---|---|---|
| | First anode-side organic layer | | | | organic layer | | organic layer | |
| | First organic | | | | | Second | | Third |
| | material | | Acceptor material | | Refractive | organic | Refractive | organic |
| | | Content | | Content | index | material | index | material |
| | Name | [mass %] | Name | [mass %] | NM₁ | Name | NM₂ | Name |
| Ex. 12 | HT1-A | 97 | HA1 | 3 | 1.97 | HT1-A | 1.97 | HT2 |
| Comp. 11 | HT1-A | 97 | HA1 | 3 | 1.97 | HT1-A | 1.97 | HT2 |

| | | | | | | | Device |
|---|---|---|---|---|---|---|---|
| | Third anode-side organic layer | | | | | | evaluation |
| | | | Additive material | | | | result |
| | Third organic material | | (Fifth material) | | | Refractive | EQE |
| | Refractive | Content | | Refractive | Content | index | (relative |
| | index | [mass %] | Name | index | [mass %] | NM₃ | value) |
| Ex. 12 | 1.79 | 50 | HT2A-1 | 1.73 | 50 | 1.76 | 110% |
| Comp. 11 | 1.79 | 100 | — | — | — | 1.79 | 100% |

Due to containing the additive material (fifth material) in the third anode-side organic layer, the organic EL device of Example 12 had an improved EQE as compared with the organic EL device of Comparative 11.

### Production (6) of Organic EL Device

Organic EL devices in Examples 13 to 16 as examples of the fourth exemplary embodiment or the sixth exemplary embodiment and an organic EL device in Comparative 12 for comparison were produced as follows. The description regarding the fourth exemplary embodiment or the sixth exemplary embodiment is applied to the description regarding Examples 13 to 16 and Comparative 12.

### Example 13

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum evaporation apparatus. First, the compound HT1-B and the compound HA1 were co-deposited on a surface of the glass substrate, where the transparent electrode line was provided, to cover the transparent electrode, thereby forming a 10-nm-thick first anode-side organic layer. The ratios of the compound HT1-B and the compound HA1 in the first anode-side organic layer were 97 mass % and 3 mass %, respectively.

The compound HT1-B was vapor-deposited on the first anode-side organic layer to form a 40-nm-thick second anode-side organic layer.

The compound HT2 and the compound HT2A-2 were co-deposited on the second anode-side organic layer to form a 40-nm-thick third anode-side organic layer. The ratios of the compound HT2 and the compound HT2A-2 in the third anode-side organic layer were both 50 mass %.

The compound EBL was vapor-deposited on the third anode-side organic layer to form a 5-nm-thick fourth anode-side organic layer.

A compound BH2-B (first host material) and a compound BD-2 (first luminescent compound) were co-deposited on the fourth anode-side organic layer to form a 20-nm-thick first emitting layer. The ratios of the compound BH2-B and the compound BD-2 in the first emitting layer were 99 mass % and 1 mass %, respectively.

A compound HBL was vapor-deposited on the first emitting layer to form a 5-nm-thick first electron transporting layer (also referred to as a hole blocking layer).

A compound ET and a compound Liq were co-deposited on the first electron transporting layer to form a 25-nm-thick second electron transporting layer. The ratios of the compound ET and the compound Liq in the second electron transporting layer (ET) were both 50 mass %.

Next, ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 13 is roughly shown as follows.

$$ITO ( 130 ) / HT 1 - B : HA 1 ( 10 , 97 \% : 3 \% ) / HT 1 - B ( 40 ) / HT 2 : HT 2 A - 2 ( 40 , 50 \% : 50 \% ) / EBL ( 5 ) / BH 2 - B : BD - 2 ( 20 , 99 \% : 1 \% ) / HBL ( 5 ) / ET : Liq ( 25 , 50 \% : 50 \% ) / Yb ( 1 ) / Al ( 80 )$$

### Example 14

An organic EL device of Example 14 was produced in the same manner as the organic EL device in Example 13 except that the compound HT2A-2 as the additive material (fifth material) in the third anode-side organic layer was replaced with the additive material listed in Table 6.

### Example 15

An organic EL device of Example 15 was produced in the same manner as the organic EL device in Example 13 except that the compound HT2A-2 as the additive material (fifth material) in the second anode-side organic layer was replaced with the additive material (compound HT2A-4) listed in Table 6, and the ratios of the compound HT2 and the compound HT2A-2 in the second anode-side organic layer were changed to 70 mass % and 30 mass %, respectively.

### Example 16

An organic EL device of Example 16 was produced in the same manner as the organic EL device in Example 13 except that the compound HT2A-2 as the additive material (fifth material) in the second anode-side organic layer was replaced with the additive material listed in Table 6.

### Comparative 12

An organic EL device of Comparative 12 was produced in the same manner as the organic EL device of Example 13 except that the second anode-side organic layer was formed using only the compound HT2 without using the fifth material as the additive material. The ratio of the compound HT2 in the second anode-side organic layer in Comparative 12 was 100 mass %.

### Evaluation (6) of Organic EL Devices

The organic EL devices produced in Examples 13 to 16 and Comparative 12 were evaluated as follows. Table 6 shows the evaluation results.

### External Quantum Efficiency EQE

External quantum efficiency EQE of each of the produced organic EL devices was calculated in the same manner as in <Evaluation (4) of Organic EL Devices>. Table 6 shows relative values of EQE, which were calculated according to a numerical formula (Numerical Formula X₁₅) below.

$$EQE ( relative value ) = EQE of each Example / EQE of Comparative 12 ) \times 100 \qquad ( Numerical Formula X15 )$$

### Lifetime LT95

Lifetime LT95 of each of the produced organic EL devices was measured in the same manner as in <Evaluation (4) of Organic EL Devices>. Relative values of lifetime LT95 were calculated based on the measurement value of LT95 in each Example according to a numerical formula (Numerical Formula X25) below.

$$LT 95 ( relative value ) = ( LT 95 of each Example / LT 95 of Comparative 12 ) \times 100 \qquad ( Numerical Formula X25 )$$

TABLE 6

| | | | | | | | | | | | | | | Device |
|---|---|---|---|---|---|---|---|---|---|---|---|---|---|---|
| | | | | | | | | | | | | | | evalua- |
| | | | | | Second anode-side | | Third anode-side organic layer | | | | | | | tion |
| | First anode-side organic layer | | | | organic layer | | | | | Additive material | | | | result |
| | First organic | | | | Second | Refrac- | Third organic material | | | (Fifth material) | | | Refrac- | EQE |
| | material | | Acceptor material | | organic | tive | | Refrac- | | | Refrac- | | tive | (rela- |
| | | Content | | Content | material | index | | tive | Content | | tive | Content | index | tive |
| | Name | [mass %] | Name | [mass %] | Name | NM₂ | Name | index | [mass %] | Name | index | [mass %] | NM₃ | value) |
| Ex. 13 | HT1 B | 97 | HA1 | 3 | HT1-B | 1.99 | HT2 | 1.79 | 50 | HT2A-2 | 1.76 | 50 | 1.78 | 109% |
| Ex. 14 | HT1-B | 97 | HA1 | 3 | HT1-B | 1.99 | HT2 | 1.79 | 50 | HT2A-3 | 1.53 | 50 | 1.66 | 106% |
| Ex. 15 | HT1-B | 97 | HA1 | 3 | HT1-B | 1.99 | HT2 | 1.79 | 70 | HT2A-4 | 1.76 | 30 | 1.78 | 108% |
| Ex. 16 | HT1-B | 97 | HA1 | 3 | HT1-B | 1.99 | HT2 | 1.79 | 50 | HT2A-5 | 1.76 | 50 | 1.78 | 107% |
| Comp. | HT1-B | 97 | HA1 | 3 | HT1-B | 1.99 | HT2 | 1.79 | 100 | — | — | — | 1.79 | 100% |
| 12 | | | | | | | | | | | | | | |

Due to containing the additive material (fifth material) in the third anode-side organic layer, the organic EL devices of Examples 13 to 16 had an improved EQE as compared with the organic EL device of Comparative 12.

### Evaluation on Compounds

### Refractive Index

Refractive indices of constituent materials (compounds) forming the organic layer were measured as follows.

A measurement target material was vacuum-deposited on a glass substrate to form a film having an approximately 50 nm thickness. Using a spectroscopic ellipsometer (M-2000UI, produced by J. A. Woollam Co., Inc. (US)), the obtained sample film was irradiated with incident light (from ultraviolet light through visible light to near-infrared light) every 5 degrees in a measurement angle range of 45 degrees to 75 degrees to measure change in a deflection state of the light reflected by the sample surface. In order to improve the measurement accuracy of the extinction coefficient, a transmission spectrum in a substrate normal direction (direction perpendicular to a surface of the substrate of the organic EL device) was also measured by M-2000UI. Similarly, the same measurement was performed also on the glass substrate on which no measurement target material was vapor-deposited. The obtained measurement information was fitted using analysis software (Complete EASE) produced by J. A. Woollam Co., Inc.

Refractive indices in an in-plane direction and a normal direction, extinction coefficients in the in-plane direction and the normal direction, and an order parameter of an organic film formed on the substrate were calculated under fitting conditions of using an anisotropic model rotationally symmetric about one axis and setting a parameter MSE indicating a mean square error in the analysis software to be 3.0 or less. A peak close to the long-wavelength region of the extinction coefficient (in-plane direction) was defined as S1, and the order parameter was calculated by a peak wavelength of S1. As fitting conditions for the glass substrate, an isotropic model was used.

Typically, a film formed by vacuum-depositing a low molecular material on the substrate is rotationally symmetric about one axis extending along the substrate normal direction. When an angle formed by the substrate normal direction and a molecular axis in a thin film formed on the substrate is defined as 0 and the extinction coefficients in a substrate parallel direction (Ordinary direction) and a substrate perpendicular direction (Extra-Ordinary direction) obtained by performing the variable-angle spectroscopic ellipsometry measurement on the thin film are respectively defined as ko and ke, S′ represented by a formula below is the order parameter.

$${S}^{′} = 1 - 〈 cos 2 θ 〉 = 2 ko / ( ke + 2 k o ) = 2 / 3 ( 1 - S ) S = ( 1 / 2 ) 〈 3 cos 2 θ - 1 〉 = ( ke - ko ) / ( ke + 2 k o )$$

An evaluation method of the molecular orientation is a publicly known method, and details thereof are described in Organic Electronics, volume 10, page 127 (2009). Further, the method for forming the thin film is a vacuum deposition method.

The order parameter S′ obtained by the variable-angle spectroscopic ellipsometry measurement is 1.0 when all molecules are oriented in parallel with the substrate. When molecules are random without being oriented, the order parameter S′ is 0.66.

Herein, a value at 2.7 eV in the substrate parallel direction (Ordinary direction), from among the values measured above, is defined as a refractive index of the measurement target material. The refractive index at 2.7 eV corresponds to a refractive index at 460 nm. Herein, the refractive index at 2.7 eV in the substrate parallel direction (Ordinary direction) may be represented by noRD and the refractive index at 2.7 eV (460 nm) in the substrate perpendicular direction (Extra-Ordinary direction) may be represented by nEXT.

When a layer was formed using a constituent material containing a plurality of compounds, a refractive index of the constituent material of the layer, the layer being a film formed by co-depositing the plurality of compounds as the measurement target material on the glass substrate or a film formed by vapor-depositing a mixture containing the plurality of compounds as the measurement target material, may be measured using a spectroscopic ellipsometer in the same manner as above.

In Examples 1 to 3, since the first anode-side organic layer is a mixture containing three types of compounds, i.e., the first organic material, the second organic material, and the additive material (fourth material), a refractive index NM₁ of a constituent material contained in the first anode-side organic layer corresponds to a refractive index of this mixture.

The refractive index of the mixture can be calculated by totalizing values, each of which is a product of a refractive index of each material and a volume fraction of each material, as described in Reference Literature 1 (J. Mater. Chem., 2009, 19,8907) and Reference Literature 2 (J. Chem. Eng. Data 1992, 37, 310-313). Herein, the refractive index of the mixture is calculated by replacing a volume fraction q with a mass ration assuming that the density of the film formed from the mixture is substantially constant. For instance, the refractive index NM₄ of the first anode-side organic layer containing the first organic material, the second organic material, and the additive material (fourth material) can be calculated using a numerical formula (Numerical Formula X3) below.

$${NM}_{14} = {n}_{1} \times {M}_{1} + {n}_{2} \times {M}_{2} + {n}_{4} \times {M}_{4} \qquad ( Numerical Formula X3 )$$

In the numerical formula (Numerical Formula X3), n₁ represents the refractive index of the first organic material, n₂ represents the refractive index of the second organic material, n₄ represents the refractive index of the fourth material, M₁ represents a mass ratio of the first organic material in the mixture, M₂ represents a mass ratio of the second organic material in the mixture, and M₄ represents a mass ratio of the fourth material in the mixture. A compound whose mass ratio in the mixture is 0.15 or less is not counted for calculation of the refractive index of the mixture. A compound whose mass ratio in the mixture exceeds 0.15 is counted for calculation of the refractive index of the mixture.

For instance, a refractive index NM₁₄ of the first anode-side organic layer of the organic EL device in Example 1 is calculated as follows: NM₁₄=2.03×{0.65/(0.65+0.25)}+2.10×{0.25/(0.65+0.25)}=2.05. In this calculation, an n2×M₂ part in the numerical formula (Numerical Formula X₃) was omitted since the mass ratio of the compound HA1 in the first anode-side organic layer was 0.10. NM₁₄ was thus calculated.

A refractive index NM₁₀ of the constituent material contained in the first anode-side organic layer of the organic EL device in Example 1 when the first anode-side organic layer did not contain the fourth material was determined to be the refractive index of the first anode-side organic layer (compound HT1) since a mass ratio of the compound HA1 was 0.10.

The refractive indices NM₁₄ in Examples 1 to 3 respectively correspond to the refractive indices NM₁ in Examples 1 to 3.

The refractive index NM₁₀ in Comparative 1 corresponds to the refractive index NM₁ in Comparative 1. The refractive index NM₁₀ in Comparative 1 was determined to be the refractive index of the first anode-side organic layer (compound HT1) since the mass ratio of the compound HA1 contained in the first anode-side organic layer was 0.10.

The refractive indices NM₁ in Comparatives 2 to 4 were determined to be the respective refractive indices of the fourth material in Comparatives 2 to 4 since the mass ratio of the second organic material (compound HA1) contained in the first anode-side organic layer in each of Comparatives 2 to 4 was 0.10.

The refractive indices NM₁₀ and NM₁₄ in Examples 1 to 3 and the refractive index NM₁₀ in Comparative 1 are shown in Table 7.

TABLE 7

| | | | | Second organic | | Additive material | | | | |
|---|---|---|---|---|---|---|---|---|---|---|
| | First organic material | | | material | | (Fourth material) | | | Refractive | Refractive |
| | | Refractive | Content | | Content | | Refractive | Content | index | index |
| | Name | index | [mass %] | Name | [mass %] | Name | index | [mass %] | NM₁₀ | NM₁₄ |
| Ex. 1 | HT1 | 2.03 | 65 | HA1 | 10 | HA2-1 | 2.10 | 25 | 2.03 | 2.05 |
| Ex. 2 | HT1 | 2.03 | 65 | HA1 | 10 | HA2-2 | 2.08 | 25 | 2.03 | 2.04 |
| Ex. 3 | HT1 | 2.03 | 65 | HA1 | 10 | HA2-3 | 2.09 | 25 | 2.03 | 2.05 |
| Comp. 1 | HT1 | 2.03 | 90 | HA1 | 10 | — | — | — | 2.03 | — |

A refractive index NM₂₅ of the second anode-side organic layer containing the third organic material and the additive material (fifth material) in each of Examples 4 to 8 can be calculated using a numerical formula (Numerical Formula X₃₁) below.

NM₂₅=n₃×M₃+n₅×M₅ (Numerical Formula X₃₁)

In the numerical formula (Numerical Formula X₃₁), n₃ represents the refractive index of the third organic material, n₅ represents the refractive index of the fifth material, M₃ represents a mass ratio of the third organic material in the mixture, and M₅ represents a mass ratio of the fifth material in the mixture.

A refractive index NM₂₀ of a constituent material contained in the second anode-side organic layer of the organic EL device in each of Examples 4 to 8 when the second anode-side organic layer did not contain the fifth material was determined to be a refractive index of the third organic material (compound HT2).

The refractive indices NM₂₅ in Examples 4 to 8 respectively correspond to the refractive indices NM₂ in Examples 4 to 8.

The refractive indices NM₂₀ in Comparatives 5 to 6 respectively correspond to the refractive indices NM₂ in Comparatives 5 to 6.

The refractive indices NM₂₀ and NM₂₅ in Examples 4 to 8 and the refractive indices NM₂₀ in Comparative 5 to 6 are shown in Table 8.

TABLE 8

| | | | | Additive material | | | | |
|---|---|---|---|---|---|---|---|---|
| | Third organic material | | | (Fifth material) | | | Refractive | Refractive |
| | | Refractive | Content | | Refractive | Content | index | index |
| | Name | index | [mass %] | Name | index | [mass %] | NM₂₀ | NM₂₅ |
| Ex. 4 | HT2 | 1.79 | 50 | HT2A-1 | 1.73 | 50 | 1.79 | 1.76 |
| Ex. 5 | HT2 | 1.79 | 50 | HT2A-2 | 1.76 | 50 | 1.79 | 1.78 |
| Ex. 6 | HT2 | 1.79 | 50 | HT2A-3 | 1.53 | 50 | 1.79 | 1.66 |
| Ex. 7 | HT2 | 1.79 | 70 | HT2A-4 | 1.76 | 30 | 1.79 | 1.78 |
| Ex. 8 | HT2 | 1.79 | 50 | HT2A-5 | 1.76 | 50 | 1.79 | 1.78 |
| Comp. 5 | HT2 | 1.79 | 100 | — | — | — | 1.79 | — |
| Comp. 6 | HT2 | 1.79 | 100 | — | — | — | 1.79 | — |

In Examples 9 to 11, since the second anode-side organic layer is a mixture containing two types of compounds, i.e., the first organic material and the additive material (fourth material), the refractive index NM₂ of the constituent material contained in the second anode-side organic layer corresponds to a refractive index of this mixture.

The refractive index of the mixture is calculated in the same manner as in Examples 1 to 3. For instance, a refractive index NM₂₄ of the second anode-side organic layer containing the second organic material and the additive material (fourth material) in each of Examples 4 to 6 can be calculated using a numerical formula (Numerical Formula X₄) below.

$${NM}_{24} = {n}_{2} \times {M}_{2} + {n}_{4} \times {M}_{4} \qquad ( Numerical Formula X4 )$$

In the numerical formula (Numerical Formula X₄), n₂ represents the refractive index of the second organic material, n₄ represents the refractive index of the fourth material, M₂ represents a mass ratio of the second organic material in the mixture, and M₄ represents a mass ratio of the fourth material in the mixture, A compound whose mass ratio in the mixture is 0.15 or less is not counted for calculation of the refractive index of the mixture. A compound whose mass ratio in the mixture exceeds 0.15 is counted for calculation of the refractive index of the mixture.

For instance, the refractive index NM₂₄ of the second anode-side organic layer of the organic EL device of Example 9 is determined as follows: NM₂₄=2.03×0.75+2.10×0.25=2.05. A refractive index NM₂₀ of a constituent material contained in the second anode-side organic layer not containing the fourth material corresponds to the refractive index of the compound HT1 as the second organic material, which is 2.03.

The refractive indices NM₂₄ in Examples 9 to 11 respectively correspond to the refractive indices NM₂ in Examples 9 to 11.

The refractive index NM₂₀ in Comparative 7 corresponds to the refractive index NM₂ in Comparative 7.

The refractive index NM₂ in each of Comparatives 8 to 10 was determined to be the refractive index of the fourth material contained in the second anode-side organic layer in the corresponding one of Comparatives 8 to 10.

In Examples 9 to 11 and Comparatives 7 to 10, the first anode-side organic layer is a mixture containing two types of compounds, i.e., the first organic material and the acceptor material. However, the refractive index NM₁ was determined to be the refractive index of the first anode-side organic layer (compound HT1) since a mass ratio of the acceptor material (compound HA1) in the first anode-side organic layer was 0.10.

TABLE 9

| | | | | Additive material | | | | |
|---|---|---|---|---|---|---|---|---|
| | Second organic material | | | (Fourth material) | | | Refractive | Refractive |
| | | Refractive | Content | | Refractive | Content | index | index |
| | Name | index | [mass %] | Name | index | [mass %] | NM₂₀ | NM₂₄ |
| Ex. 9 | HT1 | 2.03 | 75 | HA2-1 | 2.10 | 25 | 2.03 | 2.05 |
| Ex. 10 | HT1 | 2.03 | 75 | HA2-2 | 2.08 | 25 | 2.03 | 2.04 |
| Ex. 11 | HT1 | 2.03 | 75 | HA2-3 | 2.09 | 25 | 2.03 | 2.05 |
| Comp. 7 | HT1 | 2.03 | 100 | — | — | — | 2.03 | — |

A refractive index NM₃₅ of the third anode-side organic layer containing the third organic material and the additive material (fifth material) in each of Examples 12 to 16 can be calculated using a numerical formula (Numerical Formula X₄₁) below.

$${NM}_{35} = {n}_{3} \times {M}_{3} + {n}_{5} \times {M}_{5} \qquad ( Numerical Formula X41 )$$

In the numerical formula (Numerical Formula X₄₁), n₃ represents the refractive index of the third organic material, n₅ represents the refractive index of the fifth material, M₃ represents a mass ratio of the third organic material in the mixture, and M₅ represents a mass ratio of the fifth material in the mixture.

A refractive index NM₃₀ of a constituent material contained in the third anode-side organic layer of the organic EL device in each of Examples 12 to 16 when the third anode-side organic layer did not contain the fifth material was determined to be a refractive index of the third organic material (compound HT2).

In Examples 12 to 16 and Comparatives 11 to 12, the first anode-side organic layer is a mixture containing two types of compounds, i.e., the first organic material and the acceptor material. However, the refractive index NM₁ was determined to be the refractive index of the first anode-side organic layer since a mass ratio of the acceptor material in the first anode-side organic layer was 0.03.

The refractive indices NM₃₅ in Examples 12 to 16 respectively correspond to the refractive indices NM₃ in Examples 12 to 16.

The refractive indices NM₃₀ in Comparatives 11 to 12 respectively correspond to the refractive indices NM₃ in Comparatives 11 to 12.

The refractive indices NM₃₀ and NM₃₅ in Examples 12 to 16 and the refractive indices NM₃₀ in Comparative 11 to 12 are shown in Table 10.

TABLE 10

| | | | | Additive material | | | | |
|---|---|---|---|---|---|---|---|---|
| | Third organic material | | | (Fifth material) | | | Refractive | Refractive |
| | | Refractive | Content | | Refractive | Content | index | index |
| | Name | index | [mass %] | Name | index | [mass %] | NM₃₀ | NM₃₅ |
| Ex. 12 | HT2 | 1.79 | 50 | HT2A-1 | 1.73 | 50 | 1.79 | 1.76 |
| Ex. 13 | HT2 | 1.79 | 50 | HT2A-2 | 1.76 | 50 | 1.79 | 1.78 |
| Ex. 14 | HT2 | 1.79 | 50 | HT2A-3 | 1.53 | 50 | 1.79 | 1.66 |
| Ex. 15 | HT2 | 1.79 | 70 | HT2A-4 | 1.76 | 30 | 1.79 | 1.78 |
| Ex. 16 | HT2 | 1.79 | 50 | HT2A-5 | 1.76 | 50 | 1.79 | 1.78 |
| Comp. 11 | HT2 | 1.79 | 100 | — | — | — | 1.79 | — |
| Comp. 12 | HT2 | 1.79 | 100 | — | — | — | 1.79 | — |

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λ_{edge} [nm] at an intersection of the tangent and the abscissa axis was assigned to a conversion equation (F2) below to calculate singlet energy.

$${S}_{1} [ eV ] = 1239.85 / {λ}_{edge} \qquad Conversion Equation ( F2 )$$

A spectrophotometer (U3310 produced by Hitachi, Ltd.) was used for measuring absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

The singlet energy of the compound BH1 was 3.31 eV.

The singlet energy of the compound BH2 was 3.01 eV.

The singlet energy of the compound BH2-A was 3.01 eV.

The singlet energy of the compound BH2-B was 3.01 eV.

The singlet energy of the compound BD was 2.78 eV.

The singlet energy of the compound BD-1 was 2.78 eV.

The singlet energy of the compound BD-2 was 2.73 eV.

### Triplet Energy T₁

A measurement target compound was dissolved in EPA (diethylether:isopentane:ethanol=5:5:2 in volume ratio) at a concentration of 10 μmol/L, and the obtained solution was put in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. It should be noted that the triplet energy T₁ may have an error of about plus or minus 0.02 eV depending on measurement conditions.

$${T}_{1} [ eV ] = 1239 \text{.85} / {λ}_{edge} \qquad Conversion Equatio n ( F 1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 manufactured by Hitachi High-Technologies Corporation was used.

The triplet energy of the compound BH1 was 2.09 eV.

The triplet energy of the compound BH2 was 1.87 eV.

The triplet energy of the compound BH2-A was 1.87 eV.

The triplet energy of the compound BD was 2.32 eV.

The triplet energy of the compound BD-1 was 2.32 eV.

The triplet energy of the compound BD-2 was 2.29 eV.

### Measurement of Maximum Fluorescence Peak Wavelength (FL-peak) and Full Width at Half Maximum

A measurement target compound was dissolved in toluene at a concentration of 4.9×10⁻⁶ mol/L to prepare a toluene solution thereof. Using a fluorescence spectrometer (spectrophotofluorometer F-7000 manufactured by Hitachi High-Tech Science Corporation), the toluene solution of the measurement target compound was excited at 390 nm, where a maximum fluorescence peak wavelength λ (unit: nm) was measured. A full width at half maximum FWHM (unit: nm) at the maximum peak obtained based on the measured fluorescence spectrum was determined as a full width at half maximum of the measurement target compound. FWHM is an abbreviation of full width at half maximum.

The fluorescence maximum peak wavelength λ of the compound BD was 455 nm.

The full width at half maximum at the maximum peak of the compound BD was 17 nm.

The maximum fluorescence peak wavelength λ of the compound BD-1 was 445 nm.

The full width at half maximum at the maximum peak of the compound BD-1 was 17 nm.

The maximum fluorescence peak wavelength λ of the compound BD-2 was 452 nm.

The full width at half maximum at the maximum peak of the compound BD-2 was 20 nm.

### Ionization Potential

Ionization potential of each compound was measured under atmosphere using a photoelectron spectroscope (“AC-3” manufactured by RIKEN KEIKI Co., Ltd.). Specifically, the material was irradiated with light and an amount of electrons generated by charge separation was measured to determine the ionization potential of the compound.

The ionization potential of the compound HT1 was 5.66 eV.

The ionization potential of the compound HT1-A was 5.60 eV.

The ionization potential of the compound HT1-B was 5.55 eV.

### EXPLANATION OF CODES

1, 1A, 1B, 1C, 1D, 1E, 1F, 1G, 1H . . . organic EL device, 10, 10A, 10B, 10C, 10D, 10E, 10F, 10G, 10H . . . organic layer, 2 . . . substrate, 3 . . . anode, 4 . . . cathode, 5 . . . first emitting region, 50 . . . emitting layer, 51 . . . first emitting layer, 52 . . . second emitting layer, 5B . . . first emitting region, 5C . . . first emitting region, 6, 6A, 6C, 6D, 6E, 6F, 6H . . . first hole transporting zone, 61, 61F . . . first anode-side organic layer, 62, 62F . . . second anode-side organic layer, 63, 63F . . . third anode-side organic layer, 64 . . . fourth anode-side organic layer, 65, 65F . . . first mixture layer, 66, 66F . . . second mixture layer, 67 . . . third mixture layer, 7 . . . first electron transporting zone, 72 . . . electron injecting layer.

The invention claimed is:

1-41. (canceled)

42. An organic electroluminescence device, comprising:
a cathode;
an anode;
a first emitting region disposed between the cathode and the anode; and
a first hole transporting zone disposed between the anode and the first emitting region, wherein
the first emitting region comprises at least one emitting layer,
the first hole transporting zone comprises at least a first anode-side organic layer, a second anode-side organic layer, and a third anode-side organic layer,
the first anode-side organic layer, the second anode-side organic layer, and the third anode-side organic layer are disposed between the anode and the first emitting region in this order from a side close to the anode,
the second anode-side organic layer comprises a second organic material,
the second organic material is an amine compound having at least one substituted or unsubstituted amino group in a molecule,
the third anode-side organic layer has a film thickness of 20 nm or more,
the third anode-side organic layer comprises a sixth organic material,
the third organic material is an amine compound having at least one substituted or unsubstituted amino group in a molecule,
at least one of the second anode-side organic or the third anode-side organic layer comprises an additive material,
the additive material comprised in the second anode-side organic layer is a fourth material,
the additive material comprised in the third anode-side organic layer is a fifth material,
the fourth material and the fifth material are each independently an organic material or a metal atom-containing material,
the second organic material, the third organic material, the fourth material, and the fifth material are mutually different, and
the second anode-side organic layer and the third anode-side organic layer satisfy a numerical formula (Numerical Formula NX1) and at least one of numerical formulae (Numerical Formula NX2 and Numerical Formula NX3) below,
$${NM}_{2} > {NM}_{3} \qquad ( Numerical Formula NX1 )$$
$${NM}_{24} > {NM}_{20} \qquad ( Numerical Formula NX2 )$$
$${NM}_{30} > {NM}_{35} \qquad ( Numerical Formula NX3 )$$
where, in the formulae (Numerical Formula NX1, Numerical Formula NX2, and Numerical Formula NX3):
NM₂ represents a refractive index of a constituent material comprised in the second anode-side organic layer;
NM₃ represents a refractive index of a constituent material comprised in the third anode-side organic layer;
NM₂₀ represents a refractive index of the constituent material comprised in the second anode-side organic layer not comprising the fourth material;
NM₂₄ represents a refractive index of the constituent material comprised in the second anode-side organic layer comprising the fourth material;
NM₃₀ represents a refractive index of the constituent material comprised in the third anode-side organic layer not comprising the fifth material; and
NM₃₅ represents a refractive index of the constituent material comprised in the third anode-side organic layer comprising the fifth material.

43. The organic electroluminescence device according to claim 42, wherein a difference NM₂-NM₃ between the refractive index NM₂ of the constituent material comprised in the second anode-side organic layer and the refractive index NM₃ of the constituent material comprised in the third anode-side organic layer satisfies a relationship of a numerical formula (Numerical Formula NX4) below,
$${NM}_{2} - {NM}_{3} \geq 0.05 . \qquad ( Numerical Formula NX4 )$$

44. The organic electroluminescence device according to claim 42, wherein the second anode-side organic layer comprises the second organic material and the fourth material.

45. The organic electroluminescence device according to claim 42, wherein the third anode-side organic layer comprises the third organic material and the fifth material.

46. The organic electroluminescence device according to claim 42, wherein the second anode-side organic layer comprises the second organic material and the fourth material, and
the third anode-side organic layer comprises the third organic material and the fifth material.

47. The organic electroluminescence device according to claim 42, wherein a content of the fourth material in the second anode-side organic layer is 50 mass % or less.

48. The organic electroluminescence device according to claim 42, wherein a content of the fifth material in the third anode-side organic layer is 50 mass % or less.

49. The organic electroluminescence device according to claim 42, wherein
the second anode-side organic layer comprises at least one compound selected from the group consisting of a compound represented by a formula (C1) below and a compound represented by a formula (C3) below, 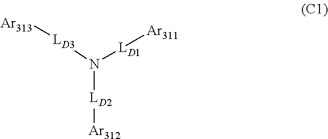
where, in the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or a group represented by —S₁(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms, 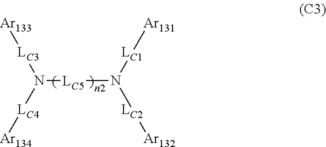
where, in the formula (C3):
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n₂ is 1, 2, 3, or 4;
when n₂ is 1, L_{C5} is a substituted or unsubstituted arylene group having 6 to 50 ring carbon atoms, or a substituted or unsubstituted divalent heterocyclic group having 5 to 50 ring atoms;
when n₂ is 2, 3, or 4, a plurality of L_{C5} are mutually the same or different;
when n₂ is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or a group represented by —S₁(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C7} are present, the plurality of R_{C2} are mutually the same or different;
when a plurality of R_{C3} are present, the plurality of R_{C3} are mutually the same or different, and
in a compound represented by the formula (C1) and a compound represented by the formula (C3), a substituent for the substituted or unsubstituted group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

50. The organic electroluminescence device according to claim 49, wherein
in the compound represented by the formula (C3), a first amino group represented by a formula (C3-1) below and a second amino group represented by a formula (C3-2) below are an identical group, 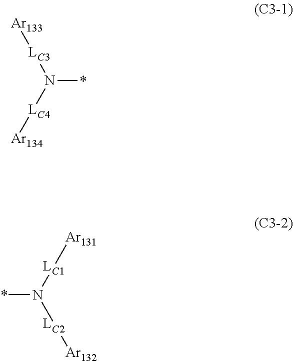
where, in the formulae (C3-1) and (C3-2), * each represent a bonding position to L_{C5}.

51. The organic electroluminescence device according to claim 49, wherein the second organic material is a compound represented by the formula (C1) or a compound represented by the formula (C3).

52. The organic electroluminescence device according to claim 42, wherein
the second anode-side organic layer comprises at least one compound selected from the group consisting of a compound represented by a formula (cHT2-1) below, a compound represented by a formula (cHT2-2) below, and a compound represented by a formula (cHT2-3) below, 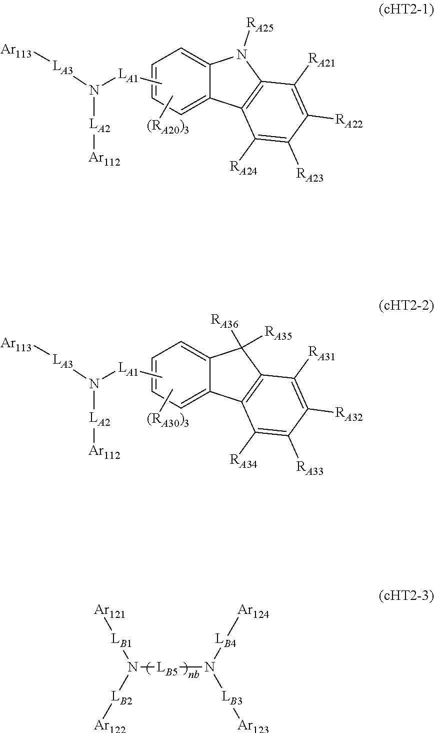
where, in the formulae (cHT2-1), (cHT2-2), and (cHT2-3):
Ar₁₁₂, Ar₁₁₃, Ar₁₂₁, Ar₁₂₂, Ar₁₂₃, and Ar₁₂₄ are each independently a substituted or unsubstituted aryl group having 6 to 50 ring carbon atoms, a substituted or unsubstituted heterocyclic group having 5 to 50 ring atoms, or a group represented by —S₁(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C7} are present, the plurality of R_{C2} are mutually the same or different;
when a plurality of R_{C3} are present, the plurality of R_{C3} are mutually the same or different;
L_{A1}, L_{A2}, L_{A3}, L_{B1}, L_{B2}, L_{B3}, and L_{B4} are each independently a single bond, a substituted or unsubstituted arylene group having 6 to 50 ring carbon atoms, or a substituted or unsubstituted divalent heterocyclic group having 5 to 50 ring atoms;
nb is 1, 2, 3 or 4;
when nb is 1, L_{B5} is a substituted or unsubstituted arylene group having 6 to 50 ring carbon atoms, or a substituted or unsubstituted divalent heterocyclic group having 5 to 50 ring atoms;
when nb is 2, 3, or 4, a plurality of L_{B5} are mutually the same or different;
when nb is 2, 3, or 4, a plurality of L_{B5} are mutually bonded to form a substituted or unsubstituted monocyclic ring, mutually bonded to form a substituted or unsubstituted fused ring, or not mutually bonded;
L_{B5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
a combination of R_{A35} and R_{A36} are mutually bonded to form a substituted or unsubstituted monocyclic ring, mutually bonded to form a substituted or unsubstituted fused ring, or not mutually bonded;
R_{A25}, and R_{A35} and R_{A36} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
at least one combination of adjacent two or more of R_{A20} to R_{A24} are mutually bonded to form a substituted or unsubstituted monocyclic ring, mutually bonded to form a substituted or unsubstituted fused ring, or not mutually bonded;
at least one combination of adjacent two or more of R_{A30} to R_{A34} are mutually bonded to form a substituted or unsubstituted monocyclic ring, mutually bonded to form a substituted or unsubstituted fused ring, or not mutually bonded;
R_{A20} to R_{A24} and R_{A30} to R_{A34} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —S₁(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
a plurality of R_{A20} are mutually the same or different;
a plurality of R_{A30} are mutually the same or different;
R₉₀₁ to R₉₀₄ in the compounds represented by the formulae (cHT2-1), (cHT2-2), and (cHT2-3) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

53. The organic electroluminescence device according to claim 52,
wherein the second organic material is a compound represented by the formula (cHT2-1), a compound represented by the formula (cHT2-2), or a compound represented by the formula (cHT2-3).

54. The organic electroluminescence device according to claim 42, wherein the second organic material is a monoamine compound having one substituted or unsubstituted amino group in a molecule.

55. The organic electroluminescence device according to claim 42, wherein the third anode-side organic layer comprises a compound represented by a formula (C1) below, 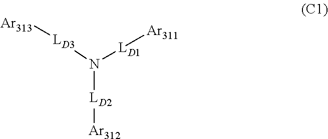
where, in the formula (C1):
Ar₃₁₁, Ar₃₁₂, and Ar₃₁₃ are each independently a substituted or unsubstituted aryl group having 6 to 50 ring carbon atoms, a substituted or unsubstituted heterocyclic group having 5 to 50 ring atoms, or a group represented by —S₁(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different; and
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms,
in the compound represented by the formula (C1), the substituent for the substituted or unsubstituted group is not a group represented by —N(R_{C6})(R_{C7}), and R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

56. The organic electroluminescence device according to claim 55, wherein the third organic material is a compound represented by the formula (C1).

57. The organic electroluminescence device according to claim 42, wherein
the third anode-side organic layer comprises at least one compound selected from the group consisting of a compound represented by a formula (cHT3-11) below, a compound represented by a formula (cHT3-2) below, a compound represented by a formula (cHT3-31) below, and a compound represented by a formula (cHT3-4) below, 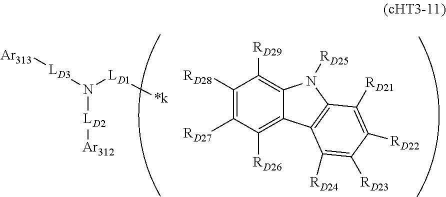 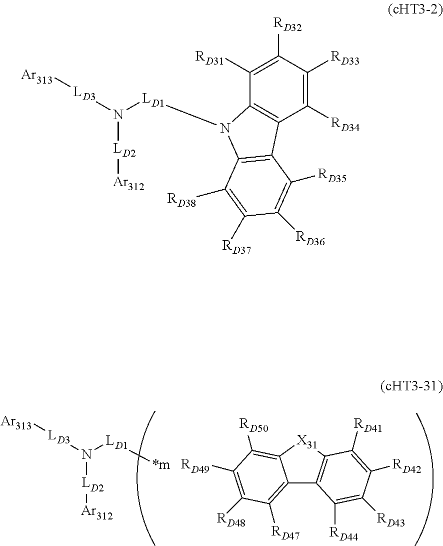 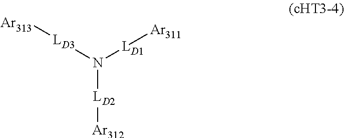
wherein, in the formulae (cHT3-11), (cHT3-2), (cHT3-31), and (cHT3-4):
Ar₃₁₁ is a group represented by one of a formula (1-a), a formula (1-b), a formula (1-c), and a formula (1-d) below;
Ar₃₁₂ and Ar₃₁₃ are each independently a substituted or unsubstituted aryl group having 6 to 50 ring carbon atoms, a substituted or unsubstituted heterocyclic group having 5 to 50 ring atoms, or a group represented by —S₁(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different;
when a plurality of R_{C3} are present, the plurality of R_{C3} are mutually the same or different;
L_{D1}, L_{D2}, and L_{D3} are each independently a single bond, a substituted or unsubstituted arylene group having 6 to 50 ring carbon atoms, or a substituted or unsubstituted divalent heterocyclic group having 5 to 50 ring atoms;
one of R_{D26} to R_{D29} is a single bond with L_{D1} and *k represents a bonding position;
at least one combination of adjacent two or more of R_{D21} to R_{D24}, and R_{D26} to R_{D29} not being the single bond with L_{D1} are mutually bonded to form a substituted or unsubstituted monocyclic ring, mutually bonded to form a substituted or unsubstituted fused ring, or not mutually bonded;
at least one combination of adjacent two or more of R_{D31} to R_{D38} are mutually bonded to form a substituted or unsubstituted monocyclic ring, mutually bonded to form a substituted or unsubstituted fused ring, or not mutually bonded;
one of R_{D47} to R_{D50} is a single bond with L_{D1} and *m represents a bonding position;
at least one combination of adjacent two or more of R_{D41} to R_{D44}, and R_{D47} to R_{D50} not being the single bond with L_{D1} are mutually bonded to form a substituted or unsubstituted monocyclic ring, mutually bonded to form a substituted or unsubstituted fused ring, or not mutually bonded;
X₃₁ is an oxygen atom, a sulfur atom, or C(R_{D45}) (R_{D46});
a combination of R_{D45} and R_{D46} are mutually bonded to form a substituted or unsubstituted monocyclic ring, mutually bonded to form a substituted or unsubstituted fused ring, or not mutually bonded;
R_{D25}, and R_{D21} to R_{D24}, R_{D26} to R_{D29}, R_{D31} to R_{D38}, and R_{D41} to R_{D50} forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —S₁(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
in the compounds represented by the formulae (cHT3-11), (cHT3-2), (cHT3-31), and (cHT3-4), R₉₀₁ to R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different, 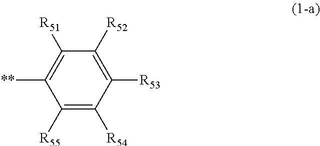
where, in the formula (1-a):
none of a combination(s) of adjacent two or more of R₅₁ to R₅₅ are bonded to each other;
R₅₁ to R₅₅ are each independently a hydrogen atom, or a substituted or unsubstituted alkyl group having 1 to 6 carbon atoms; and
** represents a bonding position to L_{D1}, 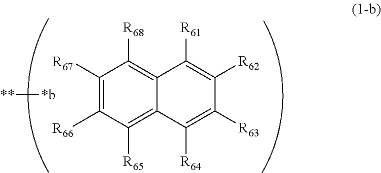
where, in the formula (1-b):
one of R₆₁ to R₆₈ is a single bond with *b;
none of a combination(s) of adjacent two or more of R₆₁ to R₆₈ not being the single bond with *b are bonded to each other;
R₆₁ to R₆₈ not being the single bond with *b are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}, 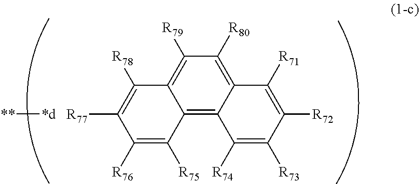
where, in the formula (1-c):
one of R₇₁ to R₈₀ is a single bond with *d;
none of a combination(s) of adjacent two or more of R₇₁ to R₈₀ not being the single bond with *d are bonded to each other;
R₇₁ to R₈₀ not being the single bond with *d are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}, 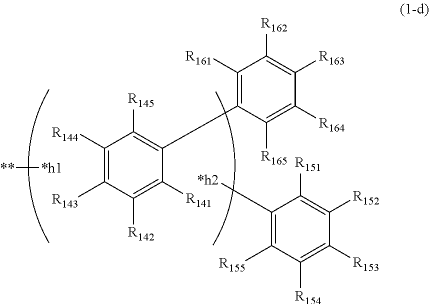
where, in the formula (1-d):
one of R₁₄₁ to R₁₄₅ is a single bond with *h1, and another one of R₁₄₁ to R₁₄₅ is a single bond with *h2;
none of a combination(s) of adjacent two or more of R₁₄₁ to R₁₄₅ not being the single bond with *h1 and not being the single bond with *h2 are bonded to each other;
at least one combination of adjacent two or more of R₁₅₁ to R₁₅₅ are mutually bonded to form a substituted or unsubstituted monocyclic ring, mutually bonded to form a substituted or unsubstituted fused ring, or not mutually bonded;
at least one combination of adjacent two or more of R₁₆₁ to R₁₆₅ are mutually bonded to form a substituted or unsubstituted monocyclic ring, mutually bonded to form a substituted or unsubstituted fused ring, or not mutually bonded;
R₁₄₁ to R₁₄₅ not being the single bond with *h1 and not being the single bond with *h2 as well as R₁₅₁ to R₁₅₅ and R₁₆₁ to R₁₆₅ forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
** represents a bonding position to L_{D1}.

58. The organic electroluminescence device according to claim 57,
wherein the third organic material is a compound represented by the formula (cHT3-11), a compound represented by the formula (cHT3-2), a compound represented by the formula (cHT3-31), or a compound represented by the formula (cHT3-4).

59. The organic electroluminescence device according to claim 42, wherein the third organic material is a monoamine compound having one substituted or unsubstituted amino group in a molecule.

60. The organic electroluminescence device according to claim 42, wherein the first anode-side organic layer comprises an acceptor material.

61. The organic electroluminescence device according to claim 60, wherein
the first anode-side organic layer comprises a first organic material,
the acceptor material and the first organic material are mutually different, and
a content of the acceptor material in the first anode-side organic layer is less than 50 mass %.

62-63. (canceled)

64. The organic electroluminescence device according to claim 42, wherein
the first anode-side organic layer comprises a sixth material as the additive material, and
the sixth material is an organic material or a metal atom-containing material.

65-66. (canceled)

67. The organic electroluminescence device according to claim 42, wherein the third anode-side organic layer has a film thickness in a range from 20 nm to 70 nm.

68. The organic electroluminescence device according to claim 42, wherein
the first hole transporting zone comprises a first mixture layer between the second anode-side organic layer and the third anode-side organic layer, and
the first mixture layer comprises the second organic material, the third organic material, and at least one of the fourth material or the fifth material as the additive material.

69. The organic electroluminescence device according to claim 42, wherein the fifth material has a refractive index of 1.80 or less.

70. The organic electroluminescence device according to claim 42, wherein the third organic material has a refractive index of 1.85 or less.

71. The organic electroluminescence device according to claim 42, wherein the third organic material has a refractive index of 1.80 or less.

72-86. (canceled)
